SEQUENCE LISTING (61 sequences)

SEQ ID NO: 1 - protein, 119 aa, synthetic construct
QVQLVQSGAEVKKPGASVKVSCKASGYTFTDYYINWVRQAPGQGLEWMGDINPYSGGPVYNQKFMARVTMTVDKSINTAYMELSRLRSDDTAVYYCARGDYYGSNYNYWGQGTLVTVSS

SEQ ID NO: 2 - protein, 107 aa, synthetic construct
EIVLTQSPATLSVSPGERATLSCRASQNIGTSIHWYQQKPGQAPRLLIKFASESISGIPARFSGSGSGTEFTLTISSLQSEDIAVYYCQQSNNWPYTFGQGTKLEIK

SEQ ID NO: 3 - protein, 8 aa, synthetic construct
GYTFTDYY

SEQ ID NO: 4 - protein, 8 aa, synthetic construct
INPYSGGP

SEQ ID NO: 5 - protein, 12 aa, synthetic construct
ARGDYYGSNYNY

SEQ ID NO: 6 - protein, 6 aa, synthetic construct
QNIGTS

SEQ ID NO: 7 - protein, 9 aa, synthetic construct
QQSNNWPYT

SEQ ID NO: 8 - protein, 121 aa, synthetic construct
QVQLVESGGGLVKPGGSLRLSCAASGFAFSTYDMFWIRQAPGKGLEWVSTISSGGGYTYYPGSVKGRFTISRDNAKNSLYLQMNSLRAEDTAVYYCVRPLLRDSYFYFDVWGQGTMVTVSS

SEQ ID NO: 9 - protein, 108 aa, synthetic construct
EIVMTQSPATLSVSPGERATLSCRASSSVSSSYLHWYQQKPGQAPRLLIFRTSNLASGIPARFSGSGSGTEYTLTISSLQSEDAAVYYCQQYSGYPLTFGQGTKLEIK

SEQ ID NO: 10 - protein, 8 aa, synthetic construct
GFAFSTYD

SEQ ID NO: 11 - protein, 8 aa, synthetic construct
ISSGGGYT

SEQ ID NO: 12 - protein, 14 aa, synthetic construct
VRPLLRDSYFYFDV

SEQ ID NO: 13 - protein, 7 aa, synthetic construct
SSVSSSY

SEQ ID NO: 14 - protein, 9 aa, synthetic construct
QQYSGYPLT

SEQ ID NO: 15 - protein, 123 aa, synthetic construct
QIQLVQSGAEVKKPGASVKVSCKASGYTFTTYGMSWVRQAPGQGLEWMGWINTHSGVPTYVDEFKGRVTMTLDTSTSTAYMELSSLRSEDTAVYYCARSPFDYGSRGAWFVYWGQGTTVTVSS

SEQ ID NO: 16 - protein, 112 aa, synthetic construct
DVVMTQSPDSLAVSLGERATINCRSSQSIVHNSGDTYLEWYQQKPGQPPKLLIYKVSNRFPGVPDRFSGSGSGTDFTLTISSLQAEDLAVYYCFQGSHVPWTFGGGTKVEIK

SEQ ID NO: 17 - protein, 13 aa, synthetic construct
KASGYTFTTYGMS

SEQ ID NO: 18 - protein, 8 aa, synthetic construct
INTHSGVP

SEQ ID NO: 19 - protein, 16 aa, synthetic construct
ARSPFDYGSRGAWFVY

SEQ ID NO: 20 - protein, 11 aa, synthetic construct
QSIVHNSGDTY

SEQ ID NO: 21 - skipped

SEQ ID NO: 22 - protein, 9 aa, synthetic construct
FQGSHVPWT

SEQ ID NO: 23 - protein, 8 aa, synthetic construct
GYTFTTYG

SEQ ID NO: 24 - protein, 8 aa, synthetic construct
INTHSGVP

SEQ ID NO: 25 - protein, 16 aa, synthetic construct
ARSPFDYGSRGAWFVY

SEQ ID NO: 26 - protein, 11 aa, synthetic construct
QSIVHNSGDTY

SEQ ID NO: 27 - protein, 9 aa, synthetic construct
FQGSHVPWT

SEQ ID NO: 28 - protein, 5 aa, synthetic construct
TYGMS

SEQ ID NO: 29 - protein, 17 aa, synthetic construct
WINTHSGVPTYVDEFKG

SEQ ID NO: 30 - protein, 14 aa, synthetic construct
SPFDYGSRGAWFVY

SEQ ID NO: 31 - protein, 16 aa, synthetic construct
RSSQSIVHNSGDTYLE

SEQ ID NO: 32 - protein, 7 aa, synthetic construct
KVSNRFP

SEQ ID NO: 33 - protein, 9 aa, synthetic construct
FQGSHVPWT

SEQ ID NO: 34 - protein, 119 aa, synthetic construct
EVQLLSSGPELVTPGASVKISCKASGYTFTDYYINWLKQSHGKSLEWIGDINPNSGGPVYNQKFMAKATLTVDKTSNTAYMELRSLTSEDSAVYYCARGDYYGSNYNYWGQGTTLTVSS

SEQ ID NO: 35 - protein, 107 aa, synthetic construct
DILLTQSPAILSVSPGERVSFSCRASQNIGTSIHWYQQRTNGSPRLLIKFASESISGIPSRFSGSGSGTDFALTINSVESEDIADYYCQQSNNWPYTFGGGTKLEIK

SEQ ID NO: 36 - protein, 8 aa, synthetic construct
GYTFTDYY

SEQ ID NO: 37 - protein, 8 aa, synthetic construct
INPNSGGP

SEQ ID NO: 38 - protein, 12 aa, synthetic construct
ARGDYYGSNYNY

SEQ ID NO: 39 - protein, 6 aa, synthetic construct
QNIGTS

SEQ ID NO: 40 - protein, 9 aa, synthetic construct
QQSNNWPYT

SEQ ID NO: 41 - protein, 121 aa, synthetic construct
EVKLVESGGGLVKPGGSLKLSCAASGFAFSTYDMFWFRQTPEKRLEWVATISSGGGYTYYPGSVKGRFTISRDNARNTLYLQMSSLRSEDTALYYCVRPLLRDSYFYFDVWGAGTTVTVSS

SEQ ID NO: 42 - protein, 108 aa, synthetic construct
DIVMTQSPAIMSTSPGEKVTMTCRASSSVSSSYLHWYQQKSGASPKLWIFRTSNLASGVPARFSGSGSGTSYSLTISSVEAEDAATYYCQQYSGYPLTFGAGTKLELK

SEQ ID NO: 43 - protein, 8 aa, synthetic construct
GFAFSTYD

SEQ ID NO: 44 - protein, 8 aa, synthetic construct
ISSGGGYT

SEQ ID NO: 45 - protein, 14 aa, synthetic construct
VRPLLRDSYFYFDV

SEQ ID NO: 46 - protein, 7 aa, synthetic construct
SSVSSSY

SEQ ID NO: 47 - protein, 9 aa, synthetic construct
QQYSGYPLT

SEQ ID NO: 48 - protein, 123 aa, synthetic construct
QIQLVQSGPELKKPGETVKISCKASGYTFTTYGMSWVKQAPGKGLKWMGWINTHSGVPTYVDEFKGRSAFSLETSASTAYLQINNLKNEDTATYFCARSPFDYGSRGAWFVYWGQGTLVTVSS

SEQ ID NO: 49 - protein, 112 aa, synthetic construct
DVLMTQTPLSLPVSLGDQASISCRSSQSIVHNNGDTYLEWYLQKPGQSPKLLIYKVSNRFPGVPDRFSGSGSGTDFTLKISRVEAEDLGLYYCFQGSHVPWTFGGGTKLEIK

SEQ ID NO: 50 - protein, 8 aa, synthetic construct
GYTFTTYG

SEQ ID NO: 51 - protein, 8 aa, synthetic construct
INTHSGVP

SEQ ID NO: 52 - protein, 16 aa, synthetic construct
ARSPFDYGSRGAWFVY

SEQ ID NO: 53 - protein, 11 aa, synthetic construct
QSIVHNNGDTY

SEQ ID NO: 54 - skipped

SEQ ID NO: 55 - protein, 9 aa, synthetic construct
FQGSHVPWT

SEQ ID NO: 56 - protein, 330 aa, synthetic construct [sequence omitted]

SEQ ID NO: 57 - protein, 107 aa, synthetic construct [sequence omitted]

SEQ ID NO: 58 - protein, 5 aa, synthetic construct
GGGGS

SEQ ID NO: 59 - protein, 6 aa, synthetic construct
HHHHHH

SEQ ID NO: 60 - protein, 4 aa, synthetic construct
GGFG

SEQ ID NO: 61 - protein, 5 aa, synthetic construct
LPXTG

(19) United States
(12) Patent Application Publication
(10) Pub. No.: US 2025/0115680 A1
(43) Pub. Date: Apr. 10, 2025

(54) PTK7 BINDING AGENTS, CONJUGATES THEREOF AND METHODS OF USING THE SAME

(71) Applicant: PROFOUNDBIO US CO., Woodinville, WA (US)

(72) Inventors: Yang XIAO, Suzhou, (CN); Lei WANG, Suzhou, (CN); Tae HAN, Woodinville, WA (US); Baiteng ZHAO, Woodinville, WA (US)

(21) Appl. No.: 18/897,786

(22) Filed: Sep. 26, 2024

(30) Foreign Application Priority Data
Sep. 26, 2023 (WO) PCT/CN2023/121540
Jul. 10, 2024 (WO) PCT/CN2024/104820

(51) Int. Cl. C07K 16/40; A61K 39/00; A61K 47/68; A61P 35/00

(52) U.S. Cl. CPC C07K 16/40; A61K 47/68037; A61K 47/6887; A61P 35/00; A61K 2039/505; C07K 2317/565; C07K 2317/567

(57) ABSTRACT

The present invention provides PTK7 antibodies, antigen binding portions thereof, other binding agents and PTK7 conjugates thereof, as well as methods and uses of such antibodies and conjugates the treatment of cancer and autoimmune disease.

### CROSS-REFERENCED TO RELATED APPLICATIONS

This application claims the benefit of International Patent Application No. PCT/CN2023/121540, filed Sep. 26, 2023, and International Patent Application No. PCT/CN2024/104820, filed Jul. 10, 2024. The entire contents of each of these applications are incorporated herein by reference in their entirety.

### SEQUENCE LISTING

The instant application contains a Sequence Listing which has been submitted electronically in XML file format and is hereby incorporated by reference in its entirety. Said XML copy, created on Sep. 26, 2024, is named GMI-408_SL.xml and is 53 KB in size.

### TECHNICAL FIELD

The present disclosure generally relates to antibodies and antibody-drug conjugates, as well as methods of using the antibodies and antibody-drug conjugates, and in particular, to such antibodies, antibody-drug conjugates, and methods related to PTK7-expressing diseases and disorders.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must be able to reach its target. If the drug is to be released after antibody drug conjugate internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models. See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

An attractive target for cancer therapies employing ADCs is protein tyrosine kinase 7 (PTK7). PTK7, also known as colon carcinoma kinase 4 (CCK4), is a member of the receptor tyrosine kinase family of proteins that transduce extracellular signals across the cell membrane. Although this protein lacks detectable catalytic tyrosine kinase activity, it is involved in the Wnt signaling pathway, and plays a role in multiple cellular processes including polarity and adhesion.

While PTK7 is found to be highly expressed in many types of cancer, it has limited expression on normal tissues in humans. This makes PTK7 an attractive target for cancer therapies. However, the pace for constructing effective PTK7 antibodies and related conjugates has been slow and the clinical trials with PTK7 antibodies and PTK7 ADCs have met with limited success thus far.

There is a need, therefore, for PTK7 antibodies generally, and for PTK7 ADCs in particular that allow for higher drug loading, but that maintain other characteristics of lower loaded conjugates, such as favorable PK properties. Embodiments of the present invention address these and related needs.

### SUMMARY

Provided herein are PTK7 binding agents, antibody drug conjugates (ADCs), and methods of using the binding agents and ADC to treatment diseases such as but not limited to cancers and autoimmune diseases.

In some embodiments, provided is a binding agent that includes a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO: 3, SEQ ID NO: 4, SEQ ID NO: 5, SEQ ID NO: 6, FAS and SEQ ID NO: 7, respectively; SEQ ID NO: 10, SEQ ID NO: 11, SEQ ID NO: 12, SEQ ID NO: 13, RTS and SEQ ID NO: 14, respectively; and SEQ ID NO: 17, SEQ ID NO: 18, SEQ ID NO: 19, SEQ ID NO: 20, KVS and SEQ ID NO: 22, respectively.

In some embodiments, provided herein is a pharmaceutical composition comprising the binding agent of the present disclosure and a pharmaceutically acceptable carrier.

In some embodiments, provided herein is a nucleic acid encoding the binding agent of the present disclosure.

In some embodiments, provided herein is a vector comprising the nucleic acid described of the present disclosure.

In some embodiments, provided herein is a vector comprising the nucleic acid of the present disclosure.

In some embodiments, provided herein is a cell line comprising the binding agent, the vector, or the nucleic acid of the present disclosure.

In some embodiments, provided herein is a conjugate that comprises the binding agent, at least one linker attached to the binding agent; at least one drug unit, wherein each drug unit is attached to a linker, wherein the linker optionally comprises at least one polar group.

In some embodiments, for the conjugate of the present disclosure, the linker is derived from a linker compound, or a stereoisomer or salt thereof, and the linker compound comprises: a linker unit; a stretcher group connected to the linker unit; an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher unit and an attachment site to the linker unit; and the linker unit has an attachment site to the amino acid unit (when present) or to the stretcher unit and to the at least one drug unit.

In some embodiments, for the conjugate of the present disclosure, the linker unit is non-cleavable.

In some embodiments, for the conjugate of the present disclosure, the linker unit is cleavable by including a cleavable group.

In some embodiments, for the conjugate of the present disclosure, the polar group is attached to the amino acid unit.

In some embodiments, for the conjugate of the present disclosure, the polar group is attached to the stretcher group.

In some embodiments, for the conjugate of the present disclosure, the linker compound comprises:
(a) the linker unit, which has from 1 to 4 attachment sites for the drug units and having one of the following structures (i) or (ii):

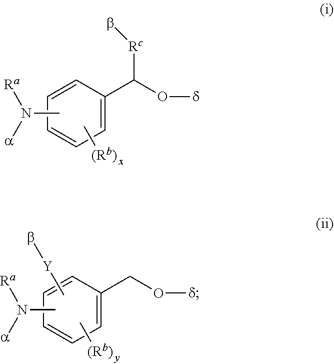

(b) the at least one polar group, each comprises a polymer unit, and
(c) the stretcher group, which has an attachment site for the binding agent; wherein:
α— is an attachment site to an enzyme-cleavable group;
β— is an attachment site to the at least one polar group;
δ— is H, an attachment site to at least one of the drug units, or an attachment site to a linking group attached to the at least one of the drug units;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the drug units, or an attachment site to at least one of the polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl or combinations thereof.

‘In some embodiments, provided herein is a pharmaceutical composition comprising the conjugate of the present disclosure and a pharmaceutically acceptable carrier.

In some embodiments, provided herein is a method of treating a PTK7+ cancer, comprising administering to a subject in need thereof a therapeutically effective amount of the binding agent, the pharmaceutical composition, the conjugate, or the pharmaceutical composition of the present disclosure.

In some embodiments, provided herein is a use of the conjugate or the pharmaceutical composition of the present disclosure for the treatment of PTK7+ cancer in a subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a graph illustrating ELISA binding assay results of humanized antibodies 2C5, 2C8, 13C4 and cofetuzumab on human PTK7;

FIG. 2 is a graph illustrating ELISA binding assay results of humanized antibodies 2C5, 2C8, 13C4 and cofetuzumab on cyano PTK7;

FIG. 3 is a graph illustrating ELISA binding assay results of humanized antibodies 2C5, 2C8, 13C4 and cofetuzumab on mouse PTK7;

FIG. 4 is a graph illustrating ELISA binding assay results of humanized antibodies 2C5, 2C8, 13C4 and cofetuzumab on rat PTK7;

FIG. 5 is a graph illustrating binding activity of antibodies to cell lines PA-1 by FACS;

FIG. 6 is a graph illustrating binding activity of antibodies to cell lines MDA-MB-468 by FACS;

FIG. 7 is a graph illustrating binding activity of antibodies to cell lines MDA-MB-231 by FACS;

FIG. 8 is a graph illustrating binding activity of antibodies to cell lines MDA-MB-453 by FACS;

FIG. 9 is a graph illustrating internalization rates of antibodies 2C5 and cofetuzumab in cell lines PA-1;

FIG. 10 is a graph illustrating internalization rates of antibodies 2C5 and cofetuzumab in cell lines MDA-MB-468;

FIG. 11 is a graph illustrating internalization rates of antibodies 2C5 and cofetuzumab in cell lines MDA-MB-453;

FIG. 12 is a graph illustrating internalization rates of antibodies 2C5 and cofetuzumab in cell lines MDA-MB-231;

FIG. 13 is a graph illustrating internalization rates of antibodies 2C5 and cofetuzumab in cell lines OVCAR-3;

FIG. 14 is a graph illustrating internalization rates of antibodies 2C8 and cofetuzumab in cell lines PA-1;

FIG. 15 is a graph illustrating internalization rates of antibodies 2C8 and cofetuzumab in cell lines MDA-MB-468;

FIG. 16 is a graph illustrating internalization rates of antibodies 2C8 and cofetuzumab in cell lines MDA-MB-453;

FIG. 17 is a graph illustrating internalization rates of antibodies 2C8 and cofetuzumab in cell lines MDA-MB-231;

FIG. 18 is a graph illustrating internalization rates of antibodies 2C8 and cofetuzumab in cell lines OVCAR-3;

FIG. 19 is a graph illustrating internalization rates of antibodies 13C4 and cofetuzumab in cell lines PA-1;

FIG. 20 is a graph illustrating internalization rates of antibodies 13C4 and cofetuzumab in cell lines MDA-MB-468;

FIG. 21 is a graph illustrating internalization rates of antibodies 13C4 and cofetuzumab in cell lines MDA-MB-453;

FIG. 22 is a graph illustrating internalization rates of antibodies 13C4 and cofetuzumab in cell lines MDA-MB-231;

FIG. 23 is a graph illustrating internalization rates of antibodies 13C4 and cofetuzumab in cell lines OVCAR-3;

FIG. 24 is a graph illustrating internalization rates of 2C8 in various tumor cell lines;

FIG. 25 is a graph illustrating internalization rates of PRO1107 in various tumor cell lines;

FIG. 26 is a graph illustrating cytotoxicity of ADCs with linker-drug LD110 on cell lines PA-1;

FIG. 27 is a graph illustrating cytotoxicity of ADCs with linker-drug LD110 on cell lines MDA-MB-468;

FIG. 28 is a graph illustrating cytotoxicity of ADCs with linker-drug LD110 on cell lines MDA-MB-453;

FIG. 29 is a graph illustrating cytotoxicity of ADCs with linker-drug LD110 on cell lines OVCAR-3;

FIG. 30 is a graph illustrating cytotoxicity of ADCs with linker-drug LD038 on tumor cell lines OVCAR-3;

FIG. 31 is a graph illustrating in vitro cell cytotoxicity of PRO1107, MMAE and b12-LD343 (8) on cell lines MDA-MB-468;

FIG. 32 is a graph illustrating in vitro cell cytotoxicity of PRO1107, MMAE and b12-LD343 (8) on cell lines OVCAR-3;

FIG. 33 is a graph illustrating in vitro cell cytotoxicity of PRO1107, MMAE and b12-LD343 (8) on cell lines PA-1;

FIG. 34 is a graph illustrating in vitro cell cytotoxicity of PRO1107, MMAE and b12-LD343 (8) on cell lines Detroit 562;

FIG. 35 is a graph illustrating in vitro cell cytotoxicity of PRO1107, MMAE and b12-LD343 (8) on cell lines AGS;

FIG. 36 is a graph illustrating in vitro cell cytotoxicity of PRO1107, MMAE and b12-LD343 (8) on cell lines SNG-M;

FIG. 37 is a graph illustrating in vitro cell cytotoxicity of PRO1107, MMAE and b12-LD343 (8) on cell lines NCI-H292;

FIG. 38 is a graph illustrating in vitro cell cytotoxicity of PRO1107, MMAE and b12-LD343 (8) on cell lines NCI-H520;

FIG. 39 is a graph illustrating in vitro cell cytotoxicity of PRO1107, MMAE and b12-LD343 (8) on cell lines SW780;

FIG. 40 is a graph illustrating PK of antibodies 2C5, 2C8, 13C4 and cofetuzumab in rat;

FIG. 41 is a graph illustrating PK of antibody 2C8 and ADC PRO1107 in rat;

FIG. 42 is a graph illustrating efficacy of 2C8 conjugates and cofetuzumab conjugates in MDA-MB-468 xenograft model;

FIG. 43 is a graph illustrating efficacy of 2C8 conjugates and cofetuzumab conjugates in PA-1 xenograft model;

FIG. 44 is a graph illustrating efficacy of other 2C8 conjugates and cofetuzumab conjugates in PA-1 xenograft model;

FIG. 45 is a graph illustrating in vivo efficacy results of PRO1107, cofetuzumab pelidotin and 2C8-vedotin (4) in PA-1 xenograft model;

FIG. 46 is a graph illustrating in vivo efficacy results of PRO1107, cofetuzumab pelidotin and 2C8-vedotin (4) in OVCAR-3 xenograft model;

FIG. 47 is a graph illustrating in vivo efficacy results of PRO1107, cofetuzumab pelidotin and 2C8-vedotin (4) in MDA-MB-468 xenograft model;

FIG. 48 is a graph illustrating in vivo efficacy results of PRO1107, cofetuzumab pelidotin and 2C8-vedotin (4) in Detroit 562 xenograft model;

FIG. 49 is a graph illustrating in vivo efficacy results of PRO1107, cofetuzumab pelidotin and 2C8-vedotin (4) in SW 780 xenograft model;

FIG. 50 is a graph illustrating in vivo efficacy results of PRO1107, cofetuzumab pelidotin and 2C8-vedotin (4) in RT4 xenograft model;

FIG. 51 is a graph illustrating in vivo efficacy results of PRO1107, cofetuzumab pelidotin and 2C8-vedotin (4) in AGS xenograft model;

FIG. 52 is a graph illustrating in vivo efficacy results of PRO1107, cofetuzumab pelidotin and 2C8-vedotin (4) in KYSE-150 xenograft model;

FIG. 53 is a graph illustrating in vivo efficacy results of PRO1107, cofetuzumab pelidotin and 2C8-vedotin (4) in NCI-H292 xenograft model;

FIG. 54A is a graph illustrating free MMAE of PRO1107, 2C8-vedotin (4) and cofetuzumab pelidotin in tumor;

FIG. 54B is a graph illustrating conjugated MMAE of PRO1107, 2C8-vedotin (4) and cofetuzumab pelidotin in tumor;

FIG. 55 shows body weight change of rats after treatment of PRO1107, 2C8-vedotin (4) and cofetuzumab pelidotin;

FIG. 56 is a graph illustrating antitumor activity of single dose of mAb-LD343 (8) and mAb-vedotin (4) in PA-1 xenograft model;

FIG. 57 is a graph illustrating antitumor activity of single dose of mAb-LD343 (8) and mAb-vedotin (4) in SW780 xenograft model;

FIG. 58 is a graph illustrating antitumor activity of single dose of mAb-LD343 (8) and mAb-vedotin (4) in OVCAR-3 xenograft model;

FIG. 59 is a graph illustrating antitumor activity of single dose of mAb-LD343 (8) and mAb-vedotin (4) in Detroit 562 xenograft model;

FIG. 60 shows the hydrophilicity of PRO1107;

FIG. 61 shows the bystander effect of PRO1107 and vedotion-based-ADCs in cell lines PA-1 and THP-1-Luc;

FIG. 62A shows tumor weight change of nude mice after treatment of PRO1107, 2C8-vedotin (4) and cofetuzumab pelidotin;

FIG. 62B shows the change of DAR value of PRO1107 in human plasma incubation;

FIG. 62C shows released payload (MMAE) at various timepoints in different species' plasma;

FIG. 63 shows the efficacy of PRO1107 in various cell-derived xenograft (CDX) models, including esophageal squamous cell carcinoma (ESCC), ovarian cancer (OVCA), breast cancer (BC), bladder cancer (BLC), gastric cancer (GC), lung cancer (LC), and head and neck squamous cell carcinoma (HNSCC); and

FIG. 64 shows the efficacy of PRO1107 in various patient-derived xenograft (CDX) models, including bladder cancer (BLC), esophageal cancer (EsC), and uterine cancer (also named endometrial cancer).

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof. Where an embodiment is set out using “comprising” language also provided is an embodiment “consisting essentially of” what is set out. Further, where an embodiment is set out using “comprising” language also provided is an embodiment “consisting of” what is set out.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

### Definitions

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

PTK7 (protein tyrosine kinase-like 7) is a member of the receptor protein tyrosine kinase family and is also known as colon carcinoma kinase 4 (CCK4). In humans, this protein is encoded by the PTK7 gene. PTK7 is overexpressed on the surface of many types of cancer, including but not limited to solid tumors. Such solid tumors include but are not limited to breast cancer (BC), lung cancer (LC), esophageal cancer (EsC), gastric cancer (GC), bladder cancer (BLC), endometrial cancer (EC), ovarian cancer (OVC), head and neck cancer (HNC) as well as hematological malignancies. Human PTK7 polypeptides include, but are not limited to, those having the amino acid sequences set forth in UniProt identifiers Q13308, and those having the amino acid sequences set forth in Genbank such as but not limited to GenBank accession numbers NP—002812.2, NP—690619.1, NP—690620.1, NP—690621.1, NP—001257327.1, XP—011513067.1, XP—011513068.1, XP—047275113.1, XP—054212039.1, XP—054212040.1, and XP—054212041.1, which are incorporated by reference herein. Although lacking detectable catalytic tyrosine kinase activity, PTK7 overexpression is intimately involved in Wnt signaling and promotes cancer cell stemness, survival and tumor progression (See e.g., Atasaven et al., 2013; Cui et al., 2021; Gärtner, 2014; Chen et al., 2014; Jiang et al., 2020; Shin et al., 2013; Liu et al., 2017; Lin et al., 2012; Özçelik et al., 2020; Xiang et al., 2022; Wang et al., 2014; Prebet et al., 2010; Jiang et al., 2012; Damelin et al., 2017). PTK7 expression in normal tissues is generally low, although some levels of protein expression have been reported in the digestive tract, such as urinary bladder, kidney, mammary gland, lung, ovary, uterus, and dendritic cells (Damelin et al., 2017). PTK7 is thus a promising target for novel anti-cancer therapy.

As used herein, an “epitope” refers to the amino acids conventionally bound by an immunoglobulin VH/VL pair, such as the antibodies, antigen binding portions thereof and other binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as human PTK7, with a KD of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the antibody, antigen binding portion or other binding agent and the concentration of target polypeptide. The person of ordinary skill in the art can determine appropriate conditions under which the antibodies, antigen binding portions and other binding agents described herein selectively bind to PTK7 using any suitable methods, such as titration of an antibody or other binding agent in a suitable cell binding assay. A binding agent specifically bound to PTK7 is not displaced by a non-similar competitor. In certain embodiments, a PTK7 antibody or antigen-binding portion thereof or other binding agent is said to specifically bind to PTK7 when it preferentially recognizes its target antigen, PTK7, in a complex mixture of proteins and/or macromolecules.

In some embodiments, a PTK7 antibody or antigen-binding portion thereof or other binding agent as described herein specifically binds to a PTK7 polypeptide with a dissociation constant (KD) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, a PTK7 antibody or antigen-binding portion thereof or other binding agent as described herein specifically binds to a PTK7 polypeptide with a dissociation constant (KD) of from about 10⁻⁵ M to 10⁻⁶ M. In some embodiments, a PTK7 antibody or antigen-binding portion thereof or other binding agent as described herein specifically binds to a PTK7 polypeptide with a dissociation constant (KD) of from about 10⁻⁷ M to 10⁻⁸ M. In some embodiments, a PTK7 antibody or antigen-binding portion thereof or other binding agent as described herein specifically binds to a PTK7 polypeptide with a dissociation constant (KD) of from about 10⁻⁸ M to 10⁻⁹ M. In some embodiments, a PTK7 antibody or antigen-binding portion thereof or other binding agent as described herein specifically binds to a PTK7 polypeptide with a dissociation constant (KD) of from about 10⁻⁹ M to 10⁻¹⁰ M. In some embodiments, a PTK7 antibody or antigen-binding portion thereof or other binding agent as described herein specifically binds to a PTK7 polypeptide with a dissociation constant (KD) of from about 10⁻¹⁰ M to 10⁻¹¹ M. In some embodiments, a PTK7 antibody or antigen-binding portion thereof or other binding agent as described herein specifically binds to a PTK7 polypeptide with a dissociation constant (KD) of from about 10⁻¹¹ M to 10⁻¹² M. In some embodiments, a PTK7 antibody or antigen-binding portion thereof or other binding agent as described herein specifically binds to a PTK7 polypeptide with a dissociation constant (KD) of from about 10⁻¹² M to 10⁻¹³ M. In some embodiments, a PTK7 antibody or antigen-binding portion thereof or other binding agent as described herein specifically binds to a PTK7 polypeptide with a dissociation constant (KD) of less than 10⁻⁹ M.

Unless otherwise indicated, the term “alkyl” by itself or as part of another term refers to a substituted or unsubstituted straight chain or branched, saturated hydrocarbon having the indicated number of carbon atoms (e.g., “—C₁-C₅ alkyl”, “—C₁-C₈ alkyl” or “—C₁-C₁₀” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)2CH(CH₃)₂), and 3,3-dimethyl-2-butyl (—CH(CH₃)C(CH₃)₃.

Unless otherwise indicated, “alkenyl” by itself or as part of another term refers to a C₂-C₈ substituted or unsubstituted straight chain or branched, hydrocarbon with at least one site of unsaturation (i.e., a carbon-carbon, sp2 double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C₂-C₈, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃, -. Up to two heteroatoms may be consecutive, such as, for example, —CH₂NHOCH₃ and CH₂OSi(CH₃)₃. In some embodiments, a C₁ to C₄ heteroalkyl has 1 to 4 carbon atoms and 1 or 2 heteroatoms and a C₁ to C₃ heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH2CH2SCH2CH2- and —CH2SCH2CH2NHCH2-. In some embodiments, a C1 to C4 heteroalkylene has 1 to 4 carbon atoms and 1 or 2 heteroatoms and a C1 to C3 heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C2 to C4 heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C₃-C₈ carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C3-C8 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C3-C8 carbocyclo”, by itself or as part of another term, refers to a substituted or unsubstituted C3-C8 carbocycle group defined above wherein another of the carbocycle groups' hydrogen atoms is replaced with a bond (i.e., it is divalent).

Unless otherwise indicated, a “C3-C10 carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C3-C10 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C3-C10 carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C3-C8 heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C3-C8 heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C3-C8 heterocyclo”, by itself or as part of another term, refers to a substituted or unsubstituted C3-C8 heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO-M+, where M+ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R₁₀, —O—, —OR₁₀, —SR₁₀, —S—, —NR₁₀₂, —NR₁₀₃, =NR₁₀, —CX₃, —CN, —OCN, —SCN, —N═C═O, —NCS, —NO, —NO₂, =N₂, —N₃, —NR₁₀C(═O)R₁₀, —C(═O)R₁₀, —C(═O)NR₁₀₂, —SO₃—, —SO₃H, —S(═O)₂R₁₀, —OS(═O)₂OR₁₀, —S(═O)₂NR₁₀, —S(═O)R₁₀, —OP(═O)(OR₁₀)₂, —P(═O)(OR₁₀)₂, —PO-3, —PO₃H₂, —AsO₂H₂, —C(═O)R₁₀, —C(═O)X, —C(═S)R₁₀, —CO₂R₁₀, —CO₂—, —C(═S)OR₁₀, C(═O)SR₁₀, C(═S)SR₁₀, C(═O)NR₁₀₂, C(═S)NR₁₀₂, or C(═NR₁₀)NR₁₀₂, where each X is independently a halogen: —F, —C, —Br, or —I; and each R₁₀ is independently —H, —C₁-C₂₀ alkyl, —C₆-C₂₀ aryl, —C₃-C₁₄ heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH₂, —N(R′)(R″), —N(R″)₃, =NR, —CX₃, —CN, —NO₂, —NR′C(═O)H, —NR′C(═O)R, —NR′C(═O)R″, —C(═O)R′, —C(═O)NH2, —C(═O)N(R′)R″, —S(═O)₂R″, —S(═O)₂NH₂, —S(═O)₂N(R′)R″, —S(═O)₂NH₂, —S(═O)₂N(R′)R″, —S(═O)₂OR′, —S(═O)R″, —OP(═O)(OR′)(OR″), —OP(OH)₃, —P(═O)(OR′)(OR″), —PO₃H₂, —C(═O)R′, —C(═S)R″, —CO₂R′, —C(═S)OR″, —C(═O)SR′, —C(═S)SR′, —C(═S)NH₂, —C(═S)N(R′)(R″)₂, —C(═NR′)NH₂, —C(═NR′)N(R′)R″, and salts thereof, wherein each X is independently selected from the group consisting of a halogen: —F, —Cl, —Br, and —I; and wherein each R″ is independently selected from the group consisting of C₁-C₂₀ alkyl, C₂-C₂₀ alkenyl, C₂-C₂₀ alkynyl, C₆-C₂₄ aryl, C3-C24 heterocyclyl (including C₅-C₂₄ heteroaryl), a protecting group, and a prodrug moiety or two of R″ together with the heteroatom to which they are attached defines a heterocyclyl; and R′ is hydrogen or R″, wherein R″ is selected from the group consisting of C1-C20 alkyl, C6-C24 aryl, C3-C24 heterocyclyl (including C5-C24 heteroaryl), and a protecting group.

Typically, optional substituents are selected from the group consisting of —X, —OH, —OR″, —SH, —SR″, —NH2, —NH(R″), —NR′(R″)₂, —N(R″)₃, =NH, =NR″, —CX3, —CN, —NO2, —NR′C(═O)H, NR′C(═O)R″, —CO2H, —C(═O)H, —C(═O)R″, —C(═O)NH2, —C(═O)NR′R″—, —S(═O)₂R″, —S(═O)2NH2, —S(═O)2N(R′)R″, —S(═O)2NH2, —S(═O)2N(R′)(R″), —S(═O)2OR′, —S(═O)R″, —C(═S)R″, —C(═S)NH2, —C(═S)N(R′)R″, —C(═NR′)N(R″)2, and salts thereof, wherein each X is independently selected from the group consisting of —F and —Cl, R″ is typically selected from the group consisting of C1-C6 alkyl, C6-C10 aryl, C3-C10 heterocyclyl (including C5-C10 heteroaryl), and a protecting group; and R′ independently is hydrogen, C1-C6 alkyl, C6-C10 aryl, C3-C10 heterocyclyl (including C5-C10 heteroaryl), and a protecting group, independently selected from R″. More typically, substituents are selected from the group consisting of —X, —R″, —OH, —OR″, —NH2, —NH(R″), —N(R″)2, —N(R″)3, —CX3, —NO2, —NHC(═O)H, —NHC(═O)R″, —C(═O)NH2, —C(═O)NHR″, —C(═O)N(R″)₂, —CO2H, —CO2R″, —C(═O)H, —C(═O)R″, —C(═O)NH2, —C(═O)NH(R″), —C(═O)N(R″)2, —C(═NR′)NH2, —C(═NR′)NH(R″), —C(═NR′)N(R″)₂, a protecting group and salts thereof, wherein each X is —F, R″ is independently selected from the group consisting of C1-C6 alkyl, C6-C10 aryl, C5-C10 heteroaryl and a protecting group; and R′ is selected from the group consisting of hydrogen, C1-C6 alkyl and a protecting group, independently selected from R″.

The compounds of the invention, or their pharmaceutically acceptable salts may contain one or more asymmetric centers and may thus give rise to enantiomers, diastereomers, and other stereoisomeric forms that are defined, in terms of absolute stereochemistry, as (R) or (S) or, as (D) or (L) for amino acids. The present invention is meant to include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another. The present invention also includes “diastereomers”, which refers to two or more stereoisomers of a compound that have different configurations at one or more of the equivalent stereocenters and are not mirror images of each other.

Although structures shown throughout the specification are depicted with specific stereocenters, the specification should be read to include variations in those stereocenters. For example, the structure of exatecan may be shown in the (S, S) configuration, but the (R, S) diastereomer of exatecan is also envisioned as being found in a separate embodiment of a conjugate as described herein.

Unless otherwise indicated, the term “drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

Unless otherwise indicated, the term “polymer unit” refers to a polymeric moiety composed of repeating subunits. Examples of polymer units include polyamides and polyethers. In some embodiments, the polymer unit is selected from an optionally substituted polyamide, a substituted polyether, or combinations thereof. In further embodiments, the polymer unit is selected from
(i) an optionally substituted polyamide comprising the formula

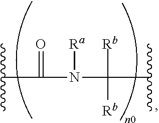

or a stereoisomer thereof, wherein each Ra is independently H or C1-6 alkyl and each Rb is independently H or C1-6 alkyl, and n0 is independently 2-26;
(ii) a substituted polyether comprising the formula

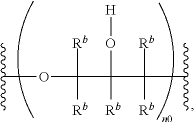

or a stereoisomer thereof, wherein each Rb is independently H or C₁₋₆ alkyl, and n0 is independently 2-26; or
(iii) combinations thereof.

Unless otherwise indicated, the term “sugar unit” or “sugar group” refers to a carbohydrate group. Examples of sugar units include glycosides.

Unless otherwise indicated, the term “carboxyl unit” or “carboxyl group” refers to a group including a carbonyl group [—C(O)—], a carboxyl group [—CO2H], and/or a carboxylate group [—CO2M, M refers to a cationic counterion].

Unless otherwise indicated, the term “stretcher group” refers to a linking moiety that connects the PTK7 binding agent to the enzyme-cleavable group.

Unless otherwise indicated, the term “polyamide” refers to polymeric groups composed of repeating subunits containing amide bonds.

Unless otherwise indicated, the term “polyether” refers to polymeric groups composed to repeating subunits containing ether bonds.

Unless otherwise indicated, the term “enzyme-cleavable group” refers to a group that is cleavable by the action of a metabolic process or reaction inside a cell or in the extracellular milieu, whereby the covalent attachment between a drug unit (e.g., a cytotoxic agent) and the linker unit or portion thereof is broken, resulting in the free drug unit, or a metabolite of the linker unit-drug, which is dissociated from the remainder of the linker unit.

The phrase “pharmaceutically acceptable salt,” as used herein, refers to pharmaceutically acceptable organic or inorganic salts of a compound (e.g., a linker, drug linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### Antibodies and Binding Agents

Provided herein are PTK7 binding antibodies (also referred to as PTK7 antibodies) and antigen binding portions thereof and other binding agents that specifically bind to human PTK7. Also provided herein are conjugates of the PTK7 antibodies and antigen binding portions and other binding agents bound to drugs, such as cytotoxic agents or immune modulatory agents (also referred to as PTK7 conjugates). In some embodiments, the PTK7 antibodies, antigen binding portions, other binding agents and/or PTK7 conjugates specifically bind to and reduce the number of PTK7+ cells in a subject. In some embodiments, the PTK7 antibodies, antigen binding portions, other binding agents and/or PTK7 conjugates specifically bind to and reduce the number of PTK7+ cancer cells in a subject. In some embodiments, the PTK7 antibodies, antigen binding portions, other binding agents and/or PTK7 conjugates specifically bind to and reduce the number of PTK7+ cells associated with a disease or condition in a subject, such as a cancer or an autoimmune disease. In some embodiments, the PTK7 antibodies, antigen binding portions, other binding agents and/or PTK7 conjugates specifically bind to and reduce the number of PTK7+ cells associated with a disease or condition in a subject, such as a human or an animal.

In some embodiments, the PTK7 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO: 35, respectively; SEQ ID NO:41 and SEQ ID NO: 42, respectively; and SEQ ID NO:48 and SEQ ID NO:49; respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:1 and SEQ ID NO:2, respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:8 and SEQ ID NO:9, respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:15 and SEQ ID NO:16, respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:34 and SEQ ID NO:35, respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:41 and SEQ ID NO:42, respectively. In some embodiments, the PTK7 antibodies or antigen binding portions thereof comprise a heavy chain variable (VH) region and a light chain variable (VL) region, the VH and VL regions having the amino acid sequences set forth in SEQ ID NO:48 and SEQ ID NO:49; respectively.

In some embodiments, the PTK7 antibodies or antigen binding portions thereof comprise a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO: 35, respectively; SEQ ID NO:41 and SEQ ID NO: 42, respectively; and SEQ ID NO:48 and SEQ ID NO:49; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO: 35, respectively; SEQ ID NO:41 and SEQ ID NO: 42, respectively; and SEQ ID NO:48 and SEQ ID NO:49; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. The phrase “wherein the CDRs of the heavy or light chain variable regions are not modified” refers to the VH and VL CDRs that do not have amino acid substitutions, deletions or insertions.

In some embodiments, the PTK7 antibodies or antigen binding portions thereof comprise a heavy chain variable (VH) region and a light chain variable (VL) region, the VH and VL regions having amino acid sequences, which have a similarity of at least 80%, at least 85%, at least 90%, at least 95%, at least 98%, or at least 99% with the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO: 35, respectively; SEQ ID NO:41 and SEQ ID NO: 42, respectively; and SEQ ID NO:48 and SEQ ID NO:49. The levels of similarities result from amino acid substitutions, deletions or insertions to the VH and VL sequences set forth in SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO: 35, respectively; SEQ ID NO:41 and SEQ ID NO: 42, respectively; and SEQ ID NO:48 and SEQ ID NO:49.

In some embodiments, the PTK7 antibodies or antigen binding portions thereof comprise a heavy chain variable (VH) region and a light chain variable (VL) region, the VH and VL regions having the amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:1 and SEQ ID NO:2, respectively.

In some embodiments, the PTK7 antibodies or antigen binding portions thereof comprise a heavy chain variable (VH) region and a light chain variable (VL) region, the VH and VL regions having the amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable region (VH) and a light chain variable region (VL), the VH and VL regions having the amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:8 and SEQ ID NO:9, respectively.

In some embodiments, the PTK7 antibodies or antigen binding portions thereof comprise a heavy chain variable (VH) region and a light chain variable (VL) region, the VH and VL regions having the amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:15 and SEQ ID NO:16, respectively.

In some embodiments, the PTK7 antibodies or antigen binding portions thereof comprise a heavy chain variable (VH) region and a light chain variable (VL) region, the VH and VL regions having the amino acid sequences set forth in SEQ ID NO:34 and SEQ ID NO:35, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in SEQ ID NO:34 and SEQ ID NO:35, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:34 and SEQ ID NO:35, respectively.

In some embodiments, the PTK7 antibodies or antigen binding portions thereof comprise a heavy chain variable (VH) region and a light chain variable (VL) region, the VH and VL regions having the amino acid sequences set forth in and SEQ ID NO:41 and SEQ ID NO:42; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in SEQ ID NO:41 and SEQ ID NO:42; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:41 and SEQ ID NO:42, respectively.

In some embodiments, the PTK7 antibodies or antigen binding portions thereof comprise a heavy chain variable (VH) region and a light chain variable (VL) region, the VH and VL regions having the amino acid sequences set forth in and SEQ ID NO:48 and SEQ ID NO:49; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof comprise a heavy chain variable (VH) region and a light chain variable (VL) region, the VH and VL regions having the amino acid sequences set forth in SEQ ID NO:48 and SEQ ID NO:49; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the PTK7 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% with the sequences set forth in SEQ ID NO:48 and SEQ ID NO:49, respectively.

In some embodiments, provided herein is a binding agent comprising a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; wherein the binding agent specifically binds to human PTK7. In some embodiments, the binding agent comprises a heavy chain variable region (VH) and a light chain variable region (VL), the VH and VL regions having the amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; wherein the binding agent specifically binds to human PTK7 with a higher or a similar binding affinity (lower Kd) than that of antibody cofetuzumab. In some embodiments, provided herein is a binding agent comprising a heavy chain variable region (VH) and a light chain variable (VL) region, the VH and VL regions having the amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes a PTK7 antibody or antigen binding portion(s) thereof and can optionally include other peptides or polypeptides covalently attached to the PTK7 antibody or antigen binding portion thereof. In any of these embodiments, the binding agent specifically binds to human PTK7.

In some embodiments, provided herein is a binding agent comprising a heavy chain variable (VH) region and a light chain variable (VL) region, the VH and VL regions having the amino acid sequences set forth in SEQ ID NO:1 and SEQ ID NO:2, respectively; wherein the binding agent specifically binds to human PTK7. In some embodiments, the binding agent comprises a heavy chain variable (VH) region and a light chain variable (VL) region, the VH and VL regions having amino acid sequences set forth SEQ ID NO:1 and SEQ ID NO:2, respectively; wherein the binding agent specifically binds to human PTK7 with a higher or a similar binding affinity (lower Kd) than that of antibody cofetuzumab. In some embodiments, provided herein is a binding agent comprising a heavy chain variable (VH) region and a light chain variable (VL) region, the VH and VL regions having the amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:8 and SEQ ID NO:9, respectively; wherein the binding agent specifically binds to human PTK7. In some embodiments, the binding agent comprises a heavy chain variable (VH) region and a light chain variable (VL) region, the VH and VL regions having the amino acid sequences set forth in SEQ ID NO:8 and SEQ ID NO:9, respectively; wherein the binding agent specifically binds to PTK7 with a higher binding affinity (lower Kd) than that of antibody cofetuzumab. In some embodiments, provided herein is a binding agent comprising a heavy chain variable (VH) region and a light chain variable (VL) region, the VH and VL regions having the amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO:15 and SEQ ID NO:16, respectively; wherein the binding agent specifically binds to human PTK7. In some embodiments, the binding agent comprises a heavy chain variable (VH) region and a light chain variable (VL) region, the VH and VL regions having the amino acid sequences set forth in SEQ ID NO:15 and SEQ ID NO:16, respectively; wherein the binding agent specifically binds to PTK7 with a higher binding affinity (lower Kd) than that of antibody cofetuzumab. In some embodiments, provided herein is a binding agent comprising a heavy chain variable (VH) region and a light chain variable (VL) region, the VH and VL regions having the amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO: 3, SEQ ID NO: 4, SEQ ID NO: 5, SEQ ID NO: 6, FAS, and SEQ ID NO: 7, respectively; (ii) SEQ ID NO: 10, SEQ ID NO: 11, SEQ ID NO: 12, SEQ ID NO: 13, RTS, and SEQ ID NO: 15, respectively; (iii) SEQ ID NO: 17, SEQ ID NO: 18, SEQ ID NO: 19, SEQ ID NO: 20, KVS and SEQ ID NO: 22, respectively; (iv) SEQ ID NO: 23, SEQ ID NO: 24, SEQ ID NO: 25, SEQ ID NO: 26, KVS, and SEQ ID NO: 27, respectively; (v) SEQ ID NO: 28, SEQ ID NO: 29, SEQ ID NO: 30, SEQ ID NO: 31, SEQ ID NO: 32 and SEQ ID NO: 33, respectively; (vi) SEQ ID NO: 36, SEQ ID NO: 37, SEQ ID NO: 38, SEQ ID NO: 39, FAS, and SEQ ID NO: 40, respectively; and (vii) SEQ ID NO: 43, SEQ ID NO: 44, SEQ ID NO: 45, SEQ ID NO: 46, RTS, and SEQ ID NO: 47, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 3, SEQ ID NO: 4, SEQ ID NO: 5, SEQ ID NO: 6, FAS, and SEQ ID NO: 7, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 10, SEQ ID NO: 11, SEQ ID NO: 12, SEQ ID NO: 13, RTS, and SEQ ID NO: 14, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 17, SEQ ID NO: 18, SEQ ID NO: 19, SEQ ID NO: 20, KVS and SEQ ID NO: 22, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:23, SEQ ID NO:24, SEQ ID NO:25, SEQ ID NO:26, KVS, and SEQ ID NO:27, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 28, SEQ ID NO: 29, SEQ ID NO: 30, SEQ ID NO: 31, SEQ ID NO: 32 and SEQ ID NO: 33, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 3, SEQ ID NO: 4, SEQ ID NO: 5, SEQ ID NO: 6, FAS and SEQ ID NO: 7; respectively. In some embodiments, each VH and VL region comprises a humanized framework region. In some embodiments, each VH and VL region comprises a human framework region. In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from (i) SEQ ID NO: 3, SEQ ID NO: 4, SEQ ID NO: 5, SEQ ID NO: 6, FAS and SEQ ID NO: 7, respectively; (ii) SEQ ID NO: 10, SEQ ID NO: 11, SEQ ID NO: 12, SEQ ID NO: 13, RTS and SEQ ID NO: 14, respectively; (iii) SEQ ID NO: 17, SEQ ID NO: 18, SEQ ID NO: 19, SEQ ID NO: 20, KVS and SEQ ID NO: 22, respectively; (iv) SEQ ID NO: 23, SEQ ID NO: 24, SEQ ID NO: 25, SEQ ID NO: 26, KVS and SEQ ID NO: 27, respectively; and (v) SEQ ID NO: 28, SEQ ID NO: 29, SEQ ID NO: 30, SEQ ID NO: 31, SEQ ID NO: 32 and SEQ ID NO: 33. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:3, SEQ ID NO:4, SEQ ID NO:5, SEQ ID NO:6, FAS and SEQ ID NO:7, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:10, SEQ ID NO:11, SEQ ID NO:12, SEQ ID NO:13, RTS and SEQ ID NO:14, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:17, SEQ ID NO:18, SEQ ID NO:19, SEQ ID NO:20, KVS and SEQ ID NO:22, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:23, SEQ ID NO:24, SEQ ID NO:25, SEQ ID NO:26, KVS and SEQ ID NO:27; respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:28, SEQ ID NO:29, SEQ ID NO:30, SEQ ID NO:31, SEQ ID NO:32 and SEQ ID NO:33, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, according to the IMGT unique numbering scheme, the VH and VL CDRs having the amino acids sequences selected from the group consisting of:
HCDR1—GYTFTTYG (SEQ ID NO: 17 & 23),
HCDR2—INTHSGVP (SEQ ID NO: 18 & 24),
HCDR3—ARSPFDYGSRGAWFVY (SEQ ID NO: 19 & 25),
LCDR1—QSIVHNSGDTY (SEQ ID NO: 20 & 26),
LCDR2—KVS,
LCDR3—FQGSHVPWT (SEQ ID NO: 22 & 27), respectively. Lefranc, M. P. The Immunologist, 7, 132-136 (1999). In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, according to the KABAT numbering scheme, the VH and VL CDRs having the amino acids sequences selected from the group consisting of:
HCDR1—TYGMS (SEQ ID NO: 28),
HCDR2—WINTHSGVPTYVDEFKG (SEQ ID NO: 29),
HCDR3—SPFDYGSRGAWFVY (SEQ ID NO: 30),
LCDR1—RSSQSIVHNSGDTYLE (SEQ ID NO: 31),
LCDR2—KVSNRFP (SEQ ID NO: 32),
LCDR3—FQGSHVPWT (SEQ ID NO: 33), respectively. DondelingerM. et al. Frontiers in Immunology, 9, 2278 (2018). In some embodiments, each VH and VL region comprises a humanized framework region. In some embodiments, each VH and VL region comprises a human framework region.

In some embodiments, provided is a binding agent comprising a heavy chain having a heavy chain variable (VH) region and a heavy chain constant region and a light chain having a light chain variable (VL) region and a light chain constant region, the VH region having the amino acid sequence set forth in SEQ ID NO: 15, the VL region having the amino acid sequence set forth in SEQ ID NO: 16, the heavy chain constant region having the amino acid sequence set forth in SEQ ID NO: 56, and the light chain constant region having the amino acid sequence set forth in SEQ ID NO: 57.

In some embodiments, the compositions and methods described herein relate to reduction of PTK7+ cells in a subject (e.g., reducing the number of PTK7+ cells in a cancer or tumor, or PTK7+ cells associated with an autoimmune disease or disorder) by a PTK7 antibody, antigen binding portion thereof, other binding agent or conjugate thereof in vivo. In some embodiments, the compositions and methods described herein relate to the treatment of PTK7+ cancer in a subject by administering a PTK7 antibody, antigen binding portion thereof, other binding agent or conjugate thereof. In some embodiments, the compositions and methods described herein relate to the treatment of an autoimmune disorder in a subject by administering a PTK7 antibody, antigen binding portion thereof, other binding agent or conjugate thereof. In some embodiments, the compositions and methods described herein relate to the treatment of disease or disorder associated with PTK7+ cells in a subject by administering a PTK7 antibody, antigen binding portion thereof, other binding agent or conjugate thereof. In any of these embodiments, the methods further include a reduction in the number of PTK7+ cells in the subject that are associated with the disease, condition or cancer.

As used herein, the term “antibody” refers to immunoglobulin molecules and immunologically active portions of immunoglobulin molecules, i.e., molecules that contain an antigen binding site(s) that specifically binds to an antigen, e.g., human PTK7. The term generally refers to antibodies comprised of two immunoglobulin heavy chain variable regions and two immunoglobulin light chain variable regions including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH4. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of a PTK7 antibody refers to the portions of a PTK7 antibody as described herein having the VH and VL sequences of the PTK7 antibody or the CDRs of a PTK7 antibody and that specifically binds to PTK7. Examples of antigen binding portions include a Fab, a Fab′, a F(ab′)2, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)2 and Fv refer to the following: (i) a Fab fragment, i.e. a monovalent fragment composed of the VL, VH, CL and CH1 domains; (ii) an F(ab′)2 fragment, i.e. a bivalent fragment comprising two Fab fragments linked to one another in the hinge region via a disulfide bridge; and (iii) an Fv fragment composed of the VL and VH domains, in each case of a PTK7 antibody. Although the two domains of the Fv fragment, namely VL and VH, are encoded by separate coding regions, they may further be linked to one another using a synthetic linker, e.g., a poly-G4S amino acid sequence (‘(G4S)n’ disclosed as SEQ ID NO: 58, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) AVHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the PTK7 antibodies or antigen binding portions thereof are part of a bispecific or multispecific binding agent. Bispecific and multi-specific antibodies include the following: an scFv1-ScFv2, an ScFv12-Fc-scFv22, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in a polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to PTK7.

In some embodiments, a conservatively modified variant of a PTK7 antibody or antigen binding portion thereof can have an alteration(s) in the framework regions (i.e., other than in the CDRs), e.g. a conservatively modified variant of a PTK7 antibody has the amino acid sequences of the VH and VL CDRs (set forth in sets of amino acid sequences (i) SEQ ID NO: 3, SEQ ID NO: 4, SEQ ID NO: 5, SEQ ID NO: 6, FAS and SEQ ID NO: 7, respectively; (ii) SEQ ID NO: 10, SEQ ID NO: 11, SEQ ID NO: 12, SEQ ID NO: 13, RTS and SEQ ID NO: 14, respectively; (iii) SEQ ID NO: 17, SEQ ID NO: 18, SEQ ID NO: 19, SEQ ID NO: 20, KVS and SEQ ID NO: 22, respectively; (iv) SEQ ID NO: 23, SEQ ID NO: 24, SEQ ID NO: 25, SEQ ID NO: 26, KVS and SEQ ID NO: 27, respectively; and (v) SEQ ID NO: 28, SEQ ID NO: 29, SEQ ID NO: 30, SEQ ID NO: 31, SEQ ID NO: 32 and SEQ ID NO: 33, respectively) and has at least one conservative amino acid substitution in a framework region (FR). In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the PTK7 antibody, antigen binding portion thereof or other binding agent exhibits specific binding to PTK7.

For conservative amino acid substitutions, a given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to PTK7.

In some embodiments, a PTK7 antibody or antigen binding portion thereof or other binding agent can be further optimized to, for example, decrease potential immunogenicity or optimize other functional property, while maintaining functional activity, for therapy in humans. In some embodiments, the PTK7 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO:35, respectively; SEQ ID NO:41 and SEQ ID NO:42, respectively, and SEQ ID NO:48 and SEQ ID NO:49; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the PTK7 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO:1 and SEQ ID NO:2, respectively; SEQ ID NO:8 and SEQ ID NO:9, respectively; SEQ ID NO:15 and SEQ ID NO:16, respectively; SEQ ID NO:34 and SEQ ID NO:35, respectively; SEQ ID NO:41 and SEQ ID NO:42, respectively, and SEQ ID NO:48 and SEQ ID NO:49; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:1 and SEQ ID NO:2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:8 and SEQ ID NO:9, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:15 and SEQ ID NO:16, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:34 and SEQ ID NO:35, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a PTK7 antibody or antigen binding portion thereof or other binding agent comprising a heavy chain variable (VH) region and a light chain variable (VL) region, the VH and VL regions having amino acid sequences set forth in SEQ ID NO:34 and SEQ ID NO:35, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a PTK7 antibody or antigen binding portion thereof or other binding agent comprising a heavy chain variable region (VH) and a light chain variable region (VL), the VH and VL regions having amino acid sequences set forth in SEQ ID NO:41 and SEQ ID NO:42; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having amino acid sequences set forth in SEQ ID NO:41 and SEQ ID NO:42; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a PTK7 antibody or antigen binding portion thereof or other binding agent comprising a heavy chain variable region (VH) and a light chain variable region (VL), the VH and VL regions having amino acid sequences set forth in SEQ ID NO:48 and SEQ ID NO:49; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having amino acid sequences set forth in SEQ ID NO:48 and SEQ ID NO:49; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In any of these embodiments, the functional activity of the PTK7 binding antibody or antigen binding portion thereof or other binding agent includes specifically binding to PTK7. Additional functional activities include depletion of PTK7+ cells (e.g., cancer cells or autoimmune cells). In the case where dose dependency does exist, it needs not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, lie; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to lie; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of a PTK7 antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g., BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, a PTK7 antibody or antigen-binding portion thereof or other binding agent has fully human constant regions. In some embodiments, a PTK7 antibody or antigen-binding portion thereof or other binding agent has humanized constant regions. In some embodiments, a PTK7 antibody or antigen-binding portion thereof or other binding agent has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgAI, and IgA2. The heavy-chain constant regions (Fc) that correspond to the different classes of immunoglobulins can be α, δ, ε, γ, and μ, respectively. The light chains can be one of either kappa (or κ) and lambda (or λ).

In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, an Fc domain can have a hybrid isotype comprising constant regions from two or more isotypes. In some embodiments, an immunoglobulin constant region can be an IgG1 or IgG4 constant region. In some embodiments, a PTK7 antibody heavy chain is of the IgG1 isotype and has the amino acid sequence set forth in SEQ ID NO:56. In some embodiments, a PTK7 antibody light chain is of the kappa isotype and has the amino acid sequence set forth in SEQ ID NO: 57.

Furthermore, a PTK7 antibody or an antigen-binding portion thereof or other binding agent may be part of a larger binding agent formed by covalent or noncovalent association of the antibody or antigen binding portion with one or more other proteins or peptides. Relevant to such binding agents are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag (‘hexahistidinyl tag’ disclosed as SEQ ID NO: 59) in order to produce bivalent and biotinylated scFv molecules (Kipriyanov, S. M., et al. (1994) Mol. Immunol. 31:10471058).

### Fc Domain Modifications to Alter Effector Function

In some embodiments, an Fc region or Fc domain of a PTK7 antibody or antigen binding portion thereof or other binding agent has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain. In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcγ receptor binding (hence likely lacking ADCC activity). The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTITM non-radioactive cytotoxicity assay for flow cytometry (CellTechnology, Inc. Mountain View, Calif.; and CytoTox 96TM non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A PTK7 antibody or antigen binding portion thereof or other binding agent with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A PTK7 antibody or antigen binding portion thereof or other binding agent with diminished binding to FcRs can be prepared containing such amino acid modifications.

In some embodiments, a PTK7 antibody or antigen binding portion thereof or other binding agent comprises an Fc domain or region with one or more amino acid substitutions which diminish FcγR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies, Antigen Binding Portions and Other Binding Agents

In various embodiments, PTK7 antibodies, antigen binding portions thereof and other binding agents can be produced in human, murine or other animal-derived cells lines. Recombinant DNA expression can be used to produce PTK7 antibodies, antigen binding portions thereof and other binding agents. This allows the production of PTK7 antibodies as well as a spectrum of PTK7 antigen binding portions and other binding agents (including fusion proteins) in a host species of choice. The production of PTK7 antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, a PTK7 VH polypeptide having the amino acid sequence set forth in SEQ ID NOs:1, 8, 15, 34, 41 or 48 is encoded by a nucleic acid. In some embodiments, a PTK7 VL polypeptide having the amino acid sequence set forth in SEQ ID NOs: 2, 9, 16, 35, 42, or 49 is encoded by a nucleic acid. In some embodiments, a nucleic acid encodes a PTK7 VH polypeptide having the amino acid sequence set forth in SEQ ID NOs: 1, 8, 15, 34, 41 or 48. In some embodiments, a nucleic acid encodes a PTK7 VL polypeptide having the amino acid sequence set forth in SEQ ID NOs: 2, 9, 16, 35, 42, or 49. In some embodiments, the nucleic acid encodes a PTK7 VH polypeptide having the amino acid sequence set forth in SEQ ID NO:1. In some embodiments, the nucleic acid encodes a PTK7 VH polypeptide having the amino acid sequence set forth in SEQ ID NO:8. In some embodiments, the nucleic acid encodes a PTK7 VH polypeptide having the amino acid sequence set forth in SEQ ID NO:15. In some embodiments, the nucleic acid encodes a PTK7 VH polypeptide having the amino acid sequence set forth in SEQ ID NO:34. In some embodiments, the nucleic acid encodes a PTK7 VH polypeptide having the amino acid sequence set forth in SEQ ID NO:41. In some embodiments, the nucleic acid encodes a PTK7 VH polypeptide having the amino acid sequence set forth in SEQ ID NO:48. In some embodiments, the nucleic acid encodes a PTK7 VH polypeptide having the amino acid sequence set forth in SEQ ID NO:2. In some embodiments, the nucleic acid encodes a PTK7 VH polypeptide having the amino acid sequence set forth in SEQ ID NO:9. In some embodiments, the nucleic acid encodes a PTK7 VH polypeptide having the amino acid sequence set forth in SEQ ID NO:16. In some embodiments, the nucleic acid encodes a PTK7 VH polypeptide having the amino acid sequence set forth in SEQ ID NO:35. In some embodiments, the nucleic acid encodes a PTK7 VH polypeptide having the amino acid sequence set forth in SEQ ID NO:42. In some embodiments, the nucleic acid encodes a PTK7 VH polypeptide having the amino acid sequence set forth in SEQ ID NO:49.

In some embodiments, the nucleic acid encodes VH and VL polypeptides having the amino acid sequences set forth in SEQ ID NOs:1 and 2. In some embodiments, the nucleic acid encodes VH and VL polypeptides having the amino acid sequences set forth in SEQ ID NOs:8 and 9. In some embodiments, the nucleic acid encodes VH and VL polypeptides having the amino acid sequences set forth in SEQ ID NOs:15 and 16. In some embodiments, the nucleic acid encodes VH and VL polypeptides having the amino acid sequences set forth in SEQ ID NOs:34 and 35. In some embodiments, the nucleic acid encodes VH and VL polypeptides having the amino acid sequences set forth in SEQ ID NOs:41 and 42. In some embodiments, the nucleic acid encodes VH and VL polypeptides having the amino acid sequences set forth in SEQ ID NOs:48 and 49.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

In some embodiments, a single nucleic acid molecule encoding both the light and heavy chains of a PTK7 antibody or antigen binding portion thereof is provided. In other embodiments, reference to a nucleic acid encoding a PTK7 antibody or antigen binding portion thereof includes one nucleic acid encoding the light chain and a separate nucleic acid encoding a heavy chain of the PTK7 antibody or antigen binding portion thereof. Similarly, in some embodiments, a single vector provided may encoded both the light and heavy chains of the PTK7 antibody or antigen binding portion thereof. Alternatively, one vector may encode the light chain and the other the heavy chain of the PTK7 antibody or antigen binding portion thereof.

Nucleic acid molecules encoding the amino acid sequence of a PTK7 antibody, antigen binding portion thereof as well as other binding agents can be prepared by a variety of methods known in the art. These methods include, but are not limited to, preparation of synthetic nucleotide sequences encoding of a PTK7 antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding a PTK7 antibody or antigen binding portion as well as other binding agents. A nucleic acid sequence encoding at least a PTK7 antibody, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode a PTK7 antibody or antigen binding portion thereof or a VH or VL polypeptide thereof or other binding agent.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., a PTK7 antibody or antigen binding portion thereof or other binding agent) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a PTK7 antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a PTK7 antibody or antigen binding portion thereof or other binding agent as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant PTK7 antibodies or antigen-binding portions thereof or other binding agents. Known glycolytic genes can also provide very efficient transcriptional control signals. For example, the promoter and terminator signals of the phosphoglycerate kinase gene can be utilized.

Production of PTK7 antibodies or antigen-binding portions thereof or other binding agents in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding a PTK7 antibody or antigen binding portion thereof or other binding agent or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding PTK7 antibodies or antigen-binding portions thereof or other binding agents include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the PTK7 variable region(s) or antigen binding portions thereof or other binding agents are then transfected singly with nucleotides encoding a PTK7 antibody or an antibody polypeptide or antigen-binding portion thereof or other binding agent, or are co-transfected with a polynucleotide(s) encoding VH and VL chain coding regions or other binding agents. The transfected recipient cells are cultured under conditions that permit expression of the incorporated coding regions and the expressed antibody chains or intact antibodies or antigen binding portions or other binding agents are recovered from the culture.

In some embodiments, the nucleic acids containing the coding regions encoding a PTK7 antibody or antigen-binding portion thereof or other binding agent are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing PTK7 antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the PTK7 antibodies or antigen binding portions thereof or other binding agents, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding a PTK7 antibody or antigen-binding portion thereof or other binding agent can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled PTK7 antibodies and antigen binding portions thereof and other binding agents. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof or other binding agents as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of PTK7 antibodies and antigen binding portions thereof and other binding agents in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof or other binding agents.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6TM cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more PTK7 antibodies or antigen-binding portions thereof or other binding agents can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H2L2 antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled PTK7 antibodies or antigen binding portions thereof or other binding agents or enhanced stability of the transfected cell lines.

Additionally, plants have emerged as a convenient, safe and economical alternative expression system for recombinant antibody production, which are based on large scale culture of microbes or animal cells. PTK7 binding antibodies or antigen binding portions thereof or other binding agents can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the PTK7 antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). A PTK7 binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to PTK7. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion or other binding agent comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion or other binding agent is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inacessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be binding agents (see, e.g. US 2006/0025576A1).

The binding agents (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the binding agents comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the binding agent a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a binding agent is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a binding agent that is a bispecific antibody is composed of a single polypeptide chain comprising two single chain FV portions (scFV) fused to each other by a peptide linker.

In some embodiments, a binding agent is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

IgG-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof and other binding agents can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure PTK7 binding antibodies or antigen binding portions thereof or other binding agents of at least about 90% to 95% homogeneity are advantageous, as are those with 98% to 99% or more homogeneity, particularly for pharmaceutical uses. Once purified, partially or to homogeneity as desired, an intact PTK7 antibody or antigen binding portions thereof or other binding agent can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Antibody Drug Conjugates

In some embodiments, a PTK7 antibody, antigen binding portion or other binding agent as described herein is part of a PTK7 antibody drug conjugate (also referred to as a PTK7 conjugate or PTK7 ADC). In some embodiments, the PTK7 antibody, antigen binding portion or other binding agent is attached to at least one linker, and at least one drug is attached to each linker. As used herein, in the context of a conjugate, the term “drug” refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a PTK7 conjugate includes at least one drug (or termed as “drug unit”) that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference. In some embodiments, the tubulin disrupting agent is MMAE.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/− dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H₂S or P2S5); C-14-alkoxymethyl(demethoxy/CH2OR) (U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide Al-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a drug is an immune modulatory agent. An immune modulatory agent can be, for example, a TLR7 and/or TLR8 agonist, a STING agonist, a RIG-1 agonist or other immune modulatory agent.

In some embodiments, a drug is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences).

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091(Novartis AG), and US20170131421 (Novartis AG).

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-I agonists include KIN1148, SB-9200, KIN700, KIN600, KIN500, KIN100, KIN101, KIN400 and KIN2000.

### Toxins

In some embodiments, a drug is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a drug is a radioactive atom. A variety of radioactive isotopes are available for the production of radioconjugates. Examples include I131, I125, Y90, Re186, Re188, Sm153, Bi213, P32, Pb212 and radioactive isotopes of Lutetium (e.g., Lu177).

### PROTACs

In some embodiments, a drug is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The PTK7 conjugates typically comprise at least one linker, each linker having at least one drug attached to it. Typically, a conjugate includes a linker between a PTK7 antibody (or antigen binding portion thereof or other binding agent) and the drug (in some cases termed “drug unit”). In various embodiments, a linker may be a protease cleavable linker, an acid-cleavable linker, a disulfide linker, a disulfide-containing linker, or a disulfide-containing linker having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690 and WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, a linker is a cleavable linker that is cleavable under intracellular conditions, such that cleavage of the linker releases the drug from the antibody (or antigen binding portion thereof or other binding agent) and/or linker in the intracellular environment. For example, in some embodiments, a linker is cleavable by a cleaving agent that is present in the intracellular environment (e.g., within a lysosome or endosome or caveolae). A linker can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, for example, in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly (SEQ ID NO: 60) linker (see, e.g., US2015/0297748). One advantage of using intracellular proteolytic release of the drug is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether linker (such as, for example, a thioether attached to the drug via an acylhydrazone bond (see, e.g., U.S. Pat. No. 5,622,929)).

In some embodiments, a linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935).

In some embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the linker is not cleavable, such as a maleimidocaproyl linker, and the drug is released by antibody degradation. (See U.S. Publication No. 2005/0238649).

In some embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC), are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, a linker promotes cellular internalization. In some embodiments, a linker promotes cellular internalization when conjugated to the drug such as a cytotoxic agent (i.e., in the milieu of the linker-drug moiety of the ADC as described herein). In yet other embodiments, a linker promotes cellular internalization when conjugated to both the drug and the PTK7 antibody (i.e., in the milieu of the ADC as described herein).

A variety of linkers that can be used with the present compositions and methods are described in WO 2004010957. In some embodiments, a protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In some embodiments, an acid cleavable linker is a hydrazine linker or a quaternary ammonium linker (see WO2017/096311 and WO2016/040684).

In some embodiments, a linker is a self-stabilizing linker comprising a maleimide group as described in U.S. Pat. No. 9,504,756.

In some embodiments, a linker is a hydrophilic linker, such as, for example, the hydrophilic peptides in WO2015/123679 and the sugar alcohol polymer-based linkers disclosed in WO2013/012961 and WO2019/213046.

In other embodiments, conjugates of a PTK7 antibody (or antigen binding portion or other binding agent) and a drug may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to an antibody, antigen binding portion thereof or other binding agent have been described in, for example WO94/11026.

The conjugates of a PTK7 antibodies (or antigen binding portion or other binding agent) include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In some embodiments, a linker is attached to a terminus of an amino acid sequence of an antibody, antigen binding portion or other binding agent or can be attached to a side chain modification of an antibody, antigen binding portion or other binding agent, such as the side chain of a lysine, serine, threonine, cysteine, tyrosine, aspartic acid, a non-natural amino acid residue, glutamine, or glutamic acid residue. An attachment between an antibody, antigen binding portion or other binding agent and a linker or drug can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, a linker is attached to an antibody, antigen binding portion or other binding agent at an interchain disulfide. In some embodiments, a linker is connected to an antibody, antigen binding portion or other binding agent at a hinge cysteine residue. In some embodiments, a linker is attached to an antibody, antigen binding portion or other binding agent at an engineered cysteine residue. In some embodiments, a linker is connected to an antibody, antigen binding portion or other binding agent at a lysine residue. In some embodiments, a linker is connected to an antibody, antigen binding portion or other binding agent at an engineered glutamine residue. In some embodiments, a linker is connected to an antibody, antigen binding portion or other binding agent at an unnatural amino acid engineered into the heavy chain.

In some embodiments, a linker is attached to an antibody, antigen binding portion or other binding agent via a sulfhydryl group. In some embodiments, a linker is attached to an antibody, antigen binding portion or other binding agent via a primary amine. In some embodiments, a linker is attached via a link created between an unnatural amino acid on an antibody, antigen binding portion or other binding agent by reacting with oxime bond that was formed by modifying a ketone group with an alkoxyamine on a drug.

In some embodiments, a linker is attached to an antibody, antigen binding portion or other binding agent via Sortase A linker. A Sortase A linker can be created by a Sortase A enzyme fusing an LPXTG recognition motif (SEQ ID NO: 61) to an N-terminal GGG motif to regenerate a native amide bond.

In some embodiments, the conjugate of the present disclosure comprises: one of the PTK7 antibodies, antigen binding portions thereof, and other binding agents, at least one linker attached to the binding agent; at least one drug unit, wherein each drug unit is attached to a linker, and wherein the linker optionally comprises at least one polar group.

In some embodiments, in the conjugate of the present disclosure, the linker is derived from a linker compound, or a stereoisomer or salt thereof, and the linker compound comprises: a linker unit; a stretcher group connected to the linker unit, an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher unit and an attachment site to the linker unit; and the linker unit has an attachment site to the amino acid unit (when present) or to the stretcher unit and to the at least one drug unit.

Some of the components and variations of the linker (and the linker compound) are exemplified and demonstrated by the “linker embodiments” herein provided.

### LINKER EMBODIMENTS

The linker (and linker compound) of the present disclosure is further illustrated by the following embodiments which should not be construed as limiting.

Embodiment 1. A linker compound, or a stereoisomer or salt thereof, comprising:
(a) a linker unit having from 1 to 4 attachment sites for a drug unit and having one of the following structures (i) or (ii):

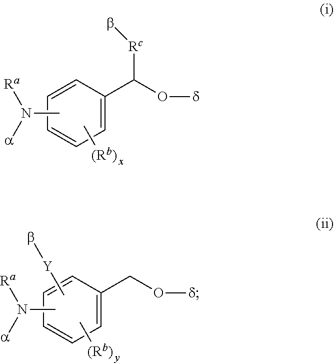

(b) at least one polar group comprising a polymer unit, optionally a sugar unit, optionally a carboxyl unit, and combinations thereof; and
(c) optionally a stretcher group having an attachment site for a PTK7 binding agent;
   wherein:
α—is an attachment site to an enzyme-cleavable group;
β—is an attachment site to the at least one polar group;
δ— is H, an attachment site to at least one of the drug units, or an attachment site to a linking group attached to the at least one of the drug units;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the drug units, or an attachment site to at least one of the polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C1-C₆ alkenylene, C1-C₆ alkynylene, triazolyl or combinations thereof.

Embodiment 2. The linker compound of Embodiment 1, wherein the linker unit has one of the following structures (i-a) or (ii-a):

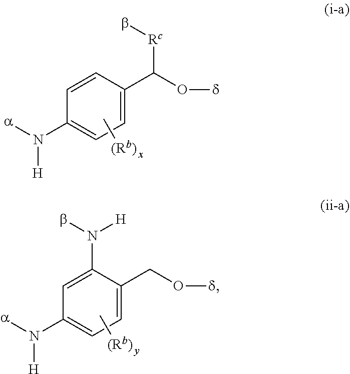

or a stereoisomer or salt thereof.

Embodiment 3. The linker compound of Embodiment 1 or 2, wherein the linker unit has one of the following structures (i-b), (i-c), (i-d), (i-e) or (i-f):

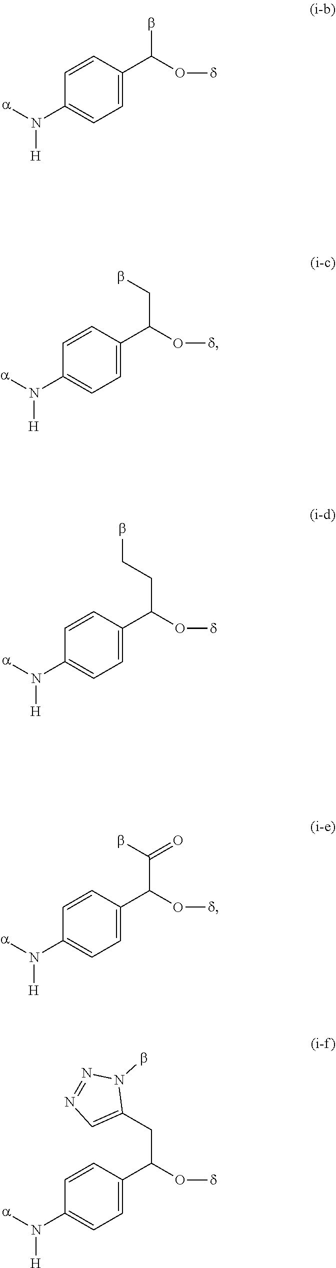

or a stereoisomer or salt thereof.

Embodiment 4. The linker compound of any of Embodiments 1-3, wherein the polar group comprises at least one sugar unit having the following formula:

L3-N(CH₂—(CH(XR))ₖ—X₁(X₂))₂ (X)
or a stereoisomer or salt thereof, wherein:
   each X is independently selected from NH and 0;
   each R is independently selected from hydrogen, acetyl, a monosaccharide, a disaccharide, and a polysaccharide;
   each X₁ is independently selected from CH₂ and C(O);
   each X₂ is independently selected from H, OH and OR;
   k is 1 to 10; and
   L3 is a point of attachment to a remainder of the polar group.

Embodiment 5. The linker compound of Embodiment 4, wherein the at least one sugar unit has one of the following structures (XII) or (XIII):

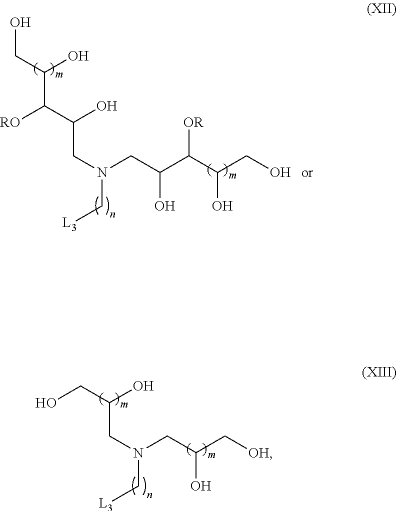

or a stereoisomer or salt thereof, wherein:
   each R is independently selected from hydrogen, a monosaccharide, a disaccharide and
a polysaccharide;
   m is 1 to 8; and
   n is 0 to 4.

Embodiment 6. The linker compound of any of Embodiments 1-5, comprising a polar group having a formula selected from:

(a)˜R²⁰—R²¹—[O—CH₂—CH₂]ₙ₂₀—R²²—NR²⁴R²⁵ (XX)
or a stereoisomer a salt thereof, wherein:
R²⁰ is an attachment group to site β or to site R^{b}, or to the enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C3-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); or —NR²⁴R²⁵ together from a C3-C₈ heterocycle; and
n20 is 2 to 26; or

(b)˜R²⁰—R²¹—[O—CH₂—CH₂]ₙ₂₀—R²²—NR²⁴R²⁵ (XXI)
or a stereoisomer or salt thereof, wherein:
R²⁰ is an attachment group to site P or to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); and the other of R²⁴ and R²⁵ is a polyethylene glycol, optionally having 1 to 24 ethylene glycol subunits; and
n20 is 2 to 26; or

(c)˜R²⁰—[—R²⁶—[R²⁹—[O—CH₂—CH₂-]ₙ₂₀R²⁹]ₙ₂₁—R²⁷—NR²⁴R²⁵]ₙ₂₇ (XXII)
or a stereoisomer or salt thereof, wherein:
R²⁰ is an attachment group to site β or to site R^{b}, or to the enzyme-cleavable group; R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)- and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); and polyethylene glycol, optionally having 1 to 24 ethylene glycol subunits; or —NR²⁴R²⁵ together from a C₃-C₈ heterocycle;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or

(d)˜R²⁰—R²¹—[—C(R^{α})H—C(O)—N(R^{N})]ₙ₂₀—R²²—NR²⁴R²⁵ (XXIII)
or a stereoisomer or salt thereof, wherein:
R²⁰ is an attachment group to site β or to site R^{b}, or to the enzyme-cleavable group;
R²¹ is a bond, C₁-C₃ alkylene,
   —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀—C(O)—;
R²² is C₁-C₃ alkylene,
   —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀—C(O)—;
each R^{α} is independently H or —R²²—NR²⁴R²⁵;
each R^{N} is independently H, C₁-C₆ alkyl or —R²²—NR²⁴R²⁵
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); or —NR²⁴R²⁵ together from a C₃-C₈ heterocycle; and
each n20 is independently 2 to 26, or

(e)˜R²⁰—R²¹—[—C(R^{α})H—C(O)—N(R^{N})—]ₙ₂₀—R²²—CO₂R²⁶ (XXIV)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene[O—CH₂—CH₂-]ₙ₂₀;
each R^{α} is independently H or —R²²—NR²⁴R²⁵;
each R^{N} is independently H, C₁-C₆ alkyl or —R²²—NR²⁴R²⁵
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); or —NR²⁴R²⁵ together from a C₃-C₈ heterocycle;
R²⁶ is H or C₁-C₄ alkyl; and
each n20 is independently 2 to 26,
with the proviso that at least one R^{α} or R^{N} is —R²²—NR²⁴R²⁵; or

(f)˜R²⁰—R²¹—[C(R^{α})H—C(O)—N(R^{N})]ₙ₂₀—R²²—N—(R²³—NR²⁴R²⁵)₂ (XXV)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene-[O—CH₂—CH₂-]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is a sugar unit of formula (XII) or (XIII); or —NR²⁴R²⁵ together from a C₃-C₈ heterocycle; and
each n20 is independently 2 to 26.

Embodiment 7. The linker compound of Embodiment 6, wherein R²⁴ and R²⁵ are each independently selected from H and a polyhydroxyl group, provided that R²⁴ and R²⁵ are not both H.

Embodiment 8. The linker compound of Embodiment 6 or 7, wherein the polyhydroxyl group is a linear monosaccharide, optionally selected from a C6 or C5 sugar, a sugar acid and an amino sugar.

Embodiment 9. The linker compound of Embodiment 8, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

Embodiment 10. The linker compound of any of Embodiments 1 to 9, wherein the attachment site β is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 11. The linker compound of any of Embodiments 1 to 10, comprising a polar group having a formula selected from the following:

(a)˜R²⁰—R²¹—[O—CH₂—CH₂]ₙ₂₀—R²²—R³⁰ (XXX)
or a stereoisomer or salt thereof, wherein:
R²⁰ is an attachment group to site β or to site R^{b}, or to the enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)— alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(b)˜R²⁰—R²¹—[O—CH₂—CH₂]ₙ₂₀—R²²—NH—C(O)—R³¹ (XXXI)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(c)˜R²⁰—R²¹—[O—CH₂—CH₂]ₙ₂₀—R²²—C(O)NH—R³¹ (XXXII)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26;

(d)˜R²⁰—R²¹—[O—CH₂—CH₂]ₙ₂₀—R²²—C(O)NR³¹—R²²—NR²⁴R²⁵ (XXXIII)
or a stereoisomer or salt thereof, wherein:
R²⁰ is an attachment group to site β or to site R^{b}, or to the enzyme-cleavable group;
R³¹ is H or R²²—NR²⁴R²⁵
R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26;

(e)—R²⁰—R²¹—[O—CH₂—CH₂]ₙ₂₀—R²²—N(R³³—R³¹)₂ (XXXIV)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(f)˜R²⁰—(R²¹—[CH₂—CH(OR³⁴)—CH₂—O]ₙ₂₀—R³⁶)ₙ₂₅ (XXXV)
or a stereoisomer or salt thereof, wherein:
R²⁰ is an attachment group to site β or to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-C₀₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
each n20 is independently 1 to 26; and
n25 is 1 or 2;

(g)˜R²⁰—R²¹—[[CH₂—CH₂—O]ₙ₂₀—R²²—[CH₂—[CH(OH)]ₙ₂₃—CH₂—O]ₙ₂₁]ₙ₂₂—R²³—NR²⁴—R²⁵ (XXXVI)
or a stereoisomer or salt thereof, wherein:
R²⁰ is an attachment group to site β or to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2;

(h)˜R²⁰—(R²¹—[O—CH₂—CH₂]ₙ₂₀—R²²—N(R^{N})—CO₂—[CH₂—CH(OR³⁴)—CH₂—O]ₙ₂₁—R³⁶)ₙ₂₅ (XXXVII)
or a stereoisomer or salt thereof, wherein:
R²⁰ is an attachment group to site β orto site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)-polyhydroxyl group; provided that both R⁴⁴ and R⁴⁵ are not H;
n20 is 2 to 26;
n21 is 1 to 26; and
n25 is 1 or 2;

(i)˜R²⁰—(R²¹—[N(R^{N})—C(O)—[O—CH₂—CH(OH)—CH₂]ₙ₂₀]ₙ₂₁—R²²—NR²⁴R²⁵)ₙ₂₅ (XXXVIII)
or a stereoisomer or salt thereof, wherein:
R²⁰ is an attachment group to site β or to site R^{b}, or to the enzyme-cleavable group;
R²¹ and R²² are each independently bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that R²⁴ and R²⁵ are not both H;
n20 is 2 to 26;
n21 is 1 to 4; and
n25 is 1, 2 or 3;

(j)˜R²⁰—(R²¹—[C(R^{α})H—C(O)—N(R^{N})]ₙ₂₀—R²²—[CH₂—CH₂—O]ₙ₂₀—NR²⁴R²⁵)ₙ₂₅ (XXXIX)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene,—C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂-]ₙ₂₀—C(O)—;
each R^{α} is independently H or —R²²—NR²⁴R²⁵;
each R^{N} is independently H, C₁-C₆ alkyl or —R²²—NR²⁴R²⁵
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, wherein R²⁸ is a sugar unit of formula (XII) or (XIII); or —NR²⁴R²⁵ together from a C₃-C₈ heterocycle), provided that R²⁴ and R²⁵ are not both H;
each n20 is independently 0 to 26, with the proviso that at least one n20 is 2 to 26; and
n25 is 1 or 2; or
(k)

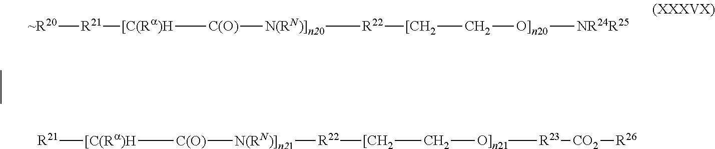

or a stereoisomer or salt thereof, wherein:
R²⁰ is an attachment group to site β or to site R^{b}, or to the enzyme-cleavable group;
R²¹, R²² and R²³ are each, independently, a bond, C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵
R²⁴ and R²⁵ are each independently selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is a sugar unit of formula (XII) or (XIII); or —NR²⁴R²⁵ together from a C₃-C₈ heterocycle), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

Embodiment 12. The linker compound of any of Embodiments 1 to 11, comprising a polar group having a formula selected from the following, or a stereoisomer or salt thereof:

˜R²⁰—R²¹—[O—CH₂—CH₂]ₙ₂₀—R²²—NH—C(O)—R³¹ (XXXI),

˜R²⁰—R²¹—[O—CH₂—CH₂]ₙ₂₀—R²²—C(O)NH—R³¹ (XXXII), and

˜R²⁰—R²¹—[O—CH₂—CH₂]ₙ₂₀—R²²—N—(R³³—R³¹)₂ (XXXIII);

wherein:
R²⁰ is an attachment group to site β or to site R^{b}, or to the enzyme-cleavable group;
R²¹ and R²² are each, independently, bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (−) line indicates an attachment site to R²⁰; and n20 is 2 to 26.

Embodiment 13. The linker compound of any of Embodiments 11 to 12, wherein the attachment to site β to site R^{b}, or to the enzyme-cleavable group is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 14. The linker compound of any of Embodiments 1-13, comprising a polar group having a formula:

˜R²⁰—(R⁴³—R⁴¹—[O—CH₂—CH₂]ₙ₄₀—R⁴²—R⁴³—(NR⁴⁴R⁴⁵)ₙ₄₁)ₙ₄₂ (XL)
or a stereoisomer or salt thereof, wherein:
R²⁰ is an attachment group to site β, to site R^{b}, or to the enzyme-cleavable group;
R⁴¹ and R⁴² are each, independently, bond or C₁-C₆ alkylene;
each R⁴³ is, independently, a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26;
n41 is 1 to 6; and
n42 is 1 to 6.

Embodiment 15. The linker compound of any of Embodiments 1-14, comprising a polar group having a formula:

˜R²⁰—(R⁴¹—[O—CH₂—CH₂]ₙ₄₀—R⁴²—R⁴³—(NR⁴⁴R⁴⁵)ₙ₄₁)ₙ₄₂ (XLI)
or a stereoisomer or salt thereof, wherein:
R²⁰ is an attachment group to site β, to site R^{b}, or to the enzyme-cleavable group;
R⁴¹ and R⁴² are each, independently, bond or C₁-C₆ alkylene;
R⁴³ is a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 26;
n41 is 1 to 6; and
n42 is 1 to 6.

Embodiment 16. The linker compound of any of Embodiments 1-15, comprising a polar group having a formula:

˜R²⁰—(R⁴¹—[O—CH₂—CH₂]ₙ₄₀—R⁴²—R⁴³—(NR⁴⁴R⁴⁵)ₙ₄₁)ₙ₄₂ (XLII)
or a stereoisomer or salt thereof, wherein:
R²⁰ is an attachment group to site β, to site R^{b}, or to the enzyme-cleavable group;
R⁴¹ and R⁴² are each, independently, bond or C₁-C₃ alkylene;
R⁴³ is a bond or is selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 16;
n41 is 1 to 4; and
n42 is 1 to 4.

Embodiment 17. The linker compound of any of Embodiments 6, 11, 12 and 14-16, wherein R²⁰ is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

Embodiment 18. The linker compound of any of Embodiments 6, 11, 12 and 14-16, wherein R²⁰ comprises one of the following structures:

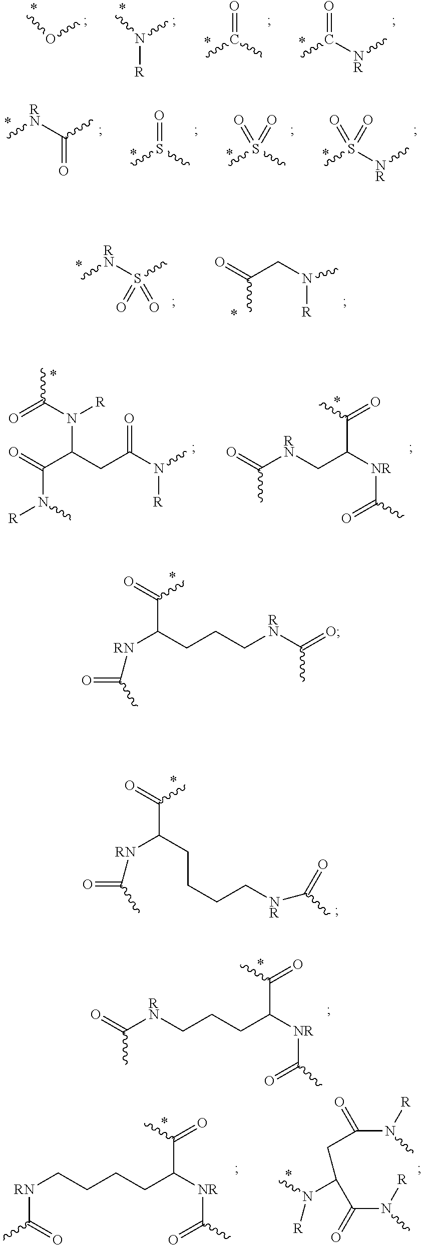

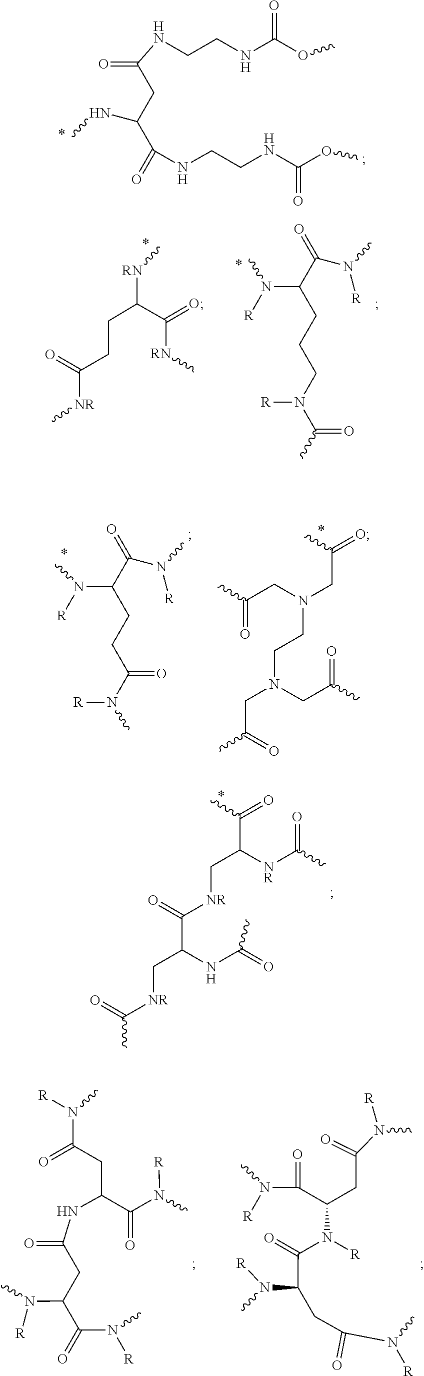

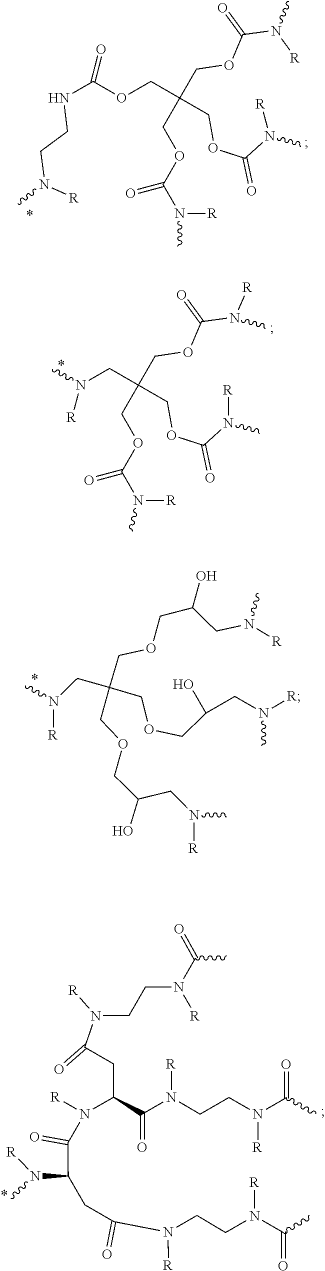

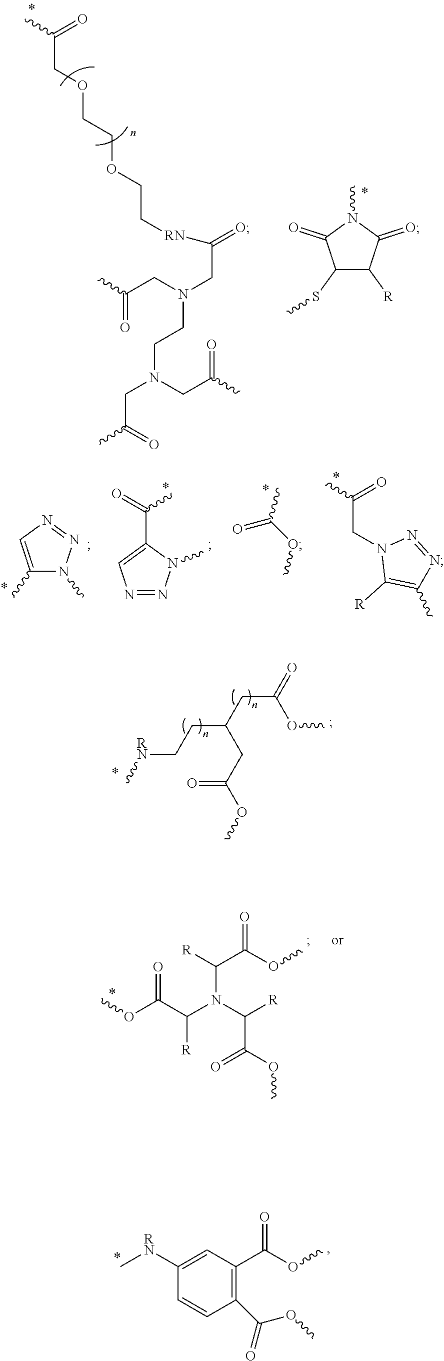

or a stereoisomer thereof, wherein R is H, C₁-C₆ alkyl or polyhydroxyl group, n is 0 to 12, the ( [symbol] *) indicates an attachment to site β or to site R^{b}, or to the enzyme-cleavable group, and the ( [symbol] ) indicates an attachment site to a remainder portion of the polar group.

Embodiment 19. The compound of any of Embodiments 6, 11, 12 and 14-16, wherein R²⁰ has one of the following structures:

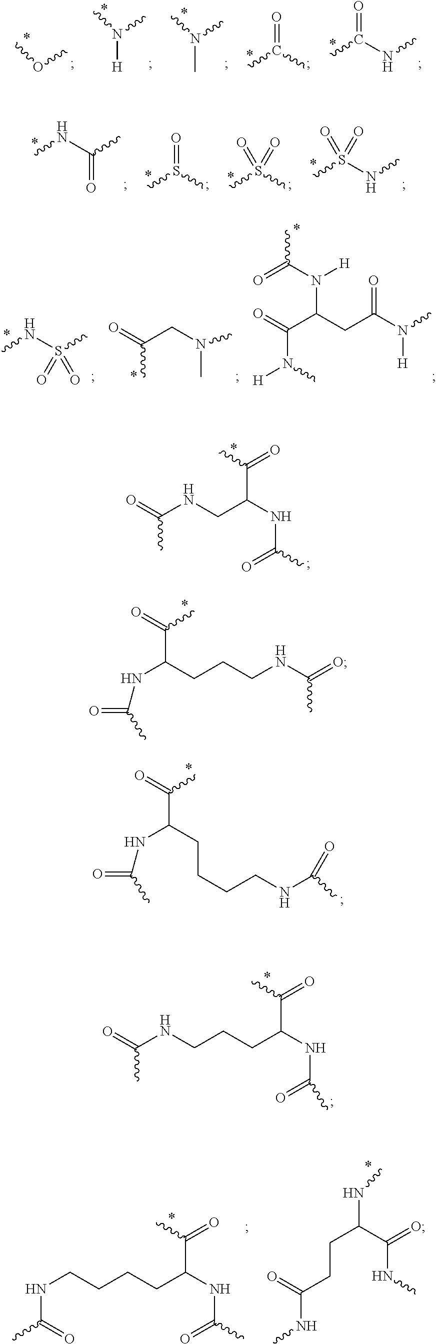

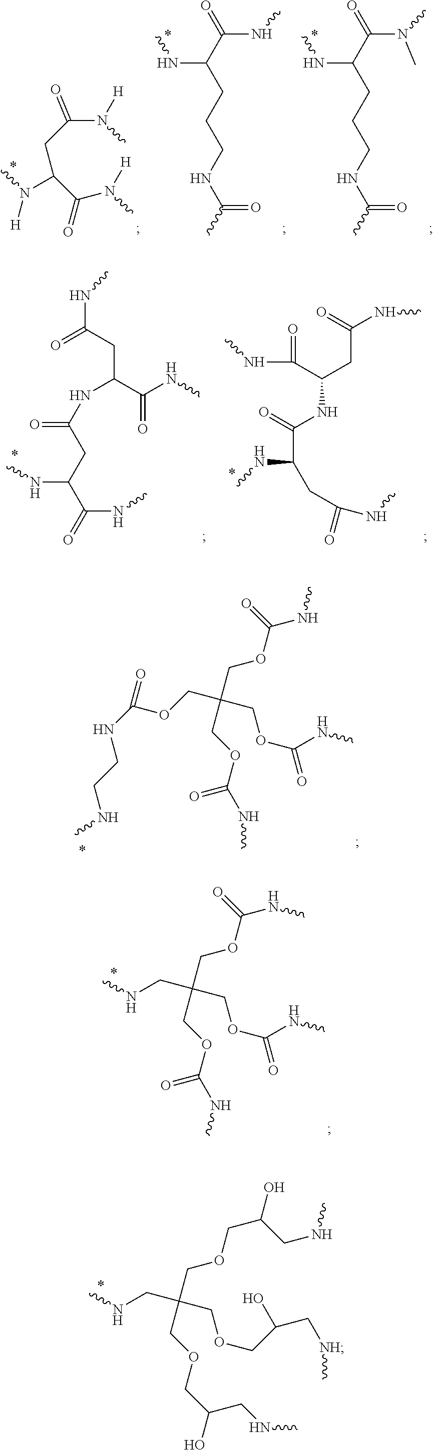

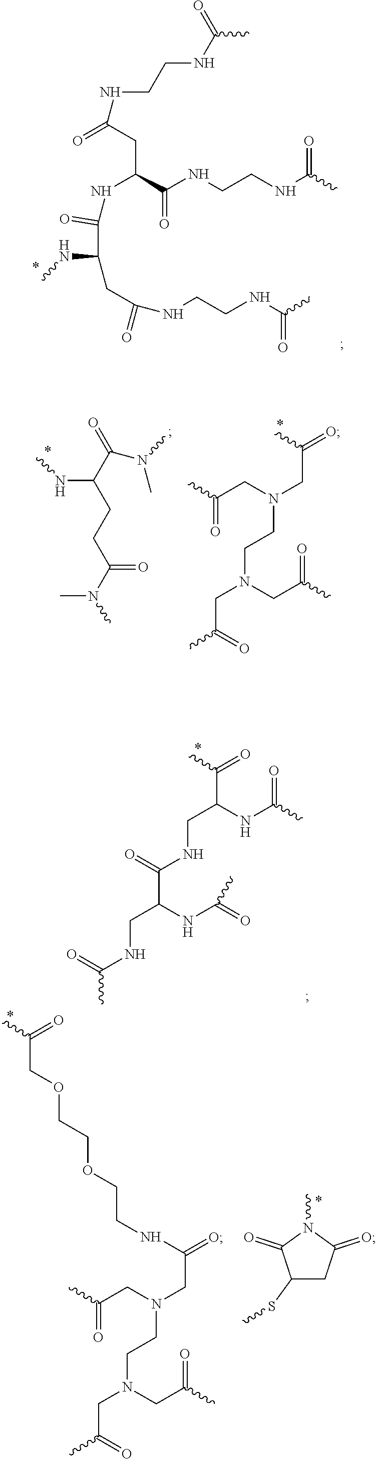

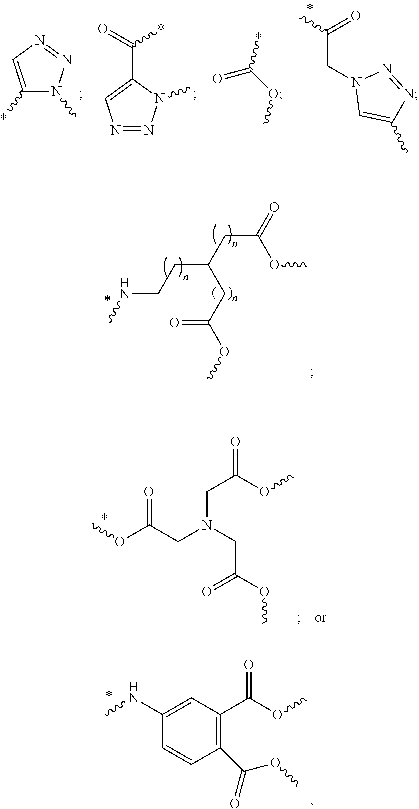

or a stereoisomer thereof, wherein n=0 to 12, the ( [symbol] *) indicates an attachment to site β or to site R^{b}, or to the enzyme-cleavable group, and the ( [symbol] ) indicates an attachment site to a remainder portion of the polar group.

Embodiment 20. The linker compound of any of Embodiments 14-19, wherein R⁴³—(NR⁴⁴R⁴⁵)ₙ₄₁ has one of the following structures:

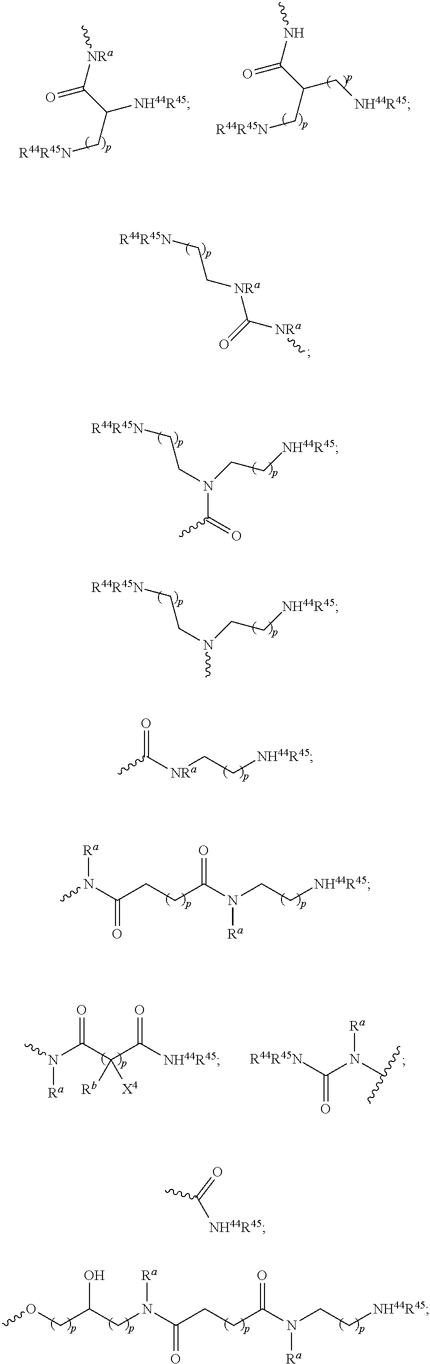

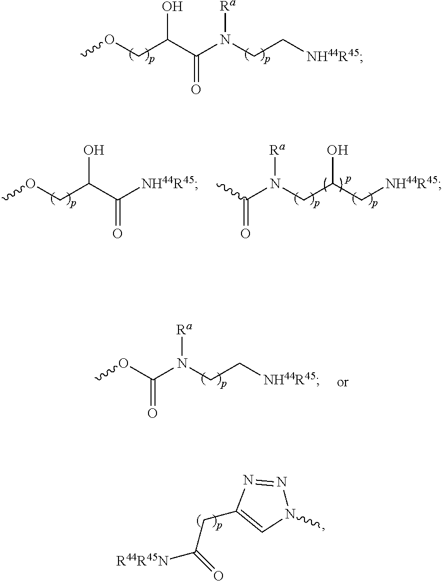

or a stereoisomer thereof, wherein R═H, C₁-C₆ alkyl, a polyhydroxyl group, or a substituted polyhydroxyl group; and the ( [symbol] ) indicates the attachment site of R⁴³ to the remainder of the polar group.

Embodiment 21. The linker compound of any of Embodiments 14-16, wherein R⁴³—(NR⁴⁴R⁴⁵)ₙ₄₁ has one of the following structures:

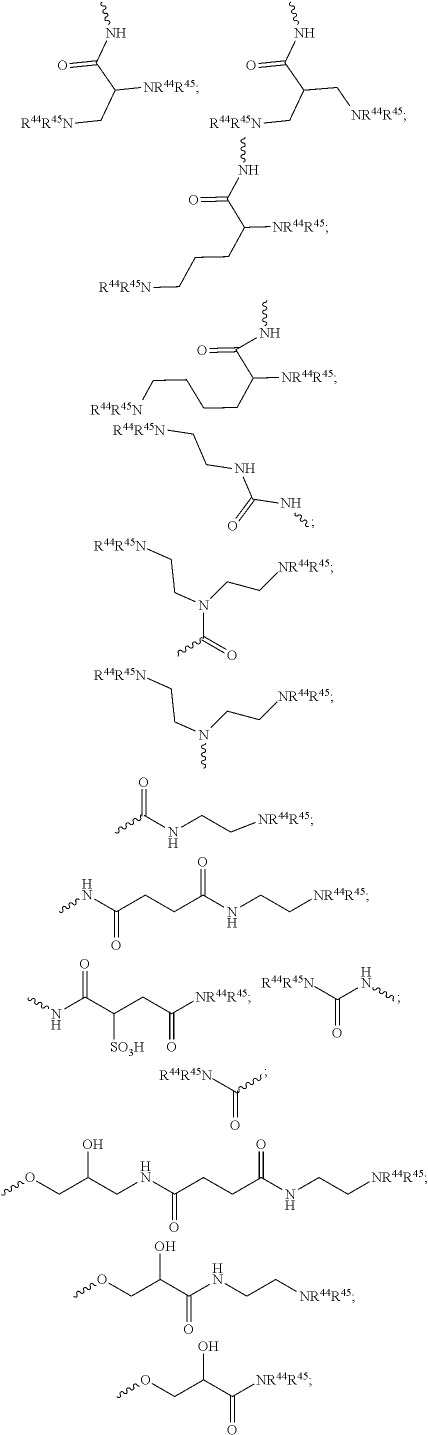

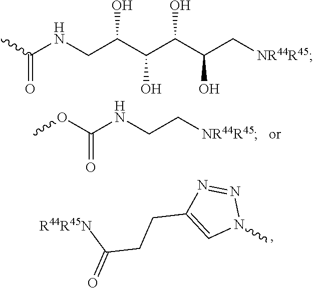

or a stereoisomer thereof, wherein the ( [symbol] ) indicates the attachment site of R⁴³ to the remainder of the polar group.

Embodiment 22. The linker compound of any of Embodiments 14-21, wherein —NR⁴⁴R⁴⁵ has one of the following structures:

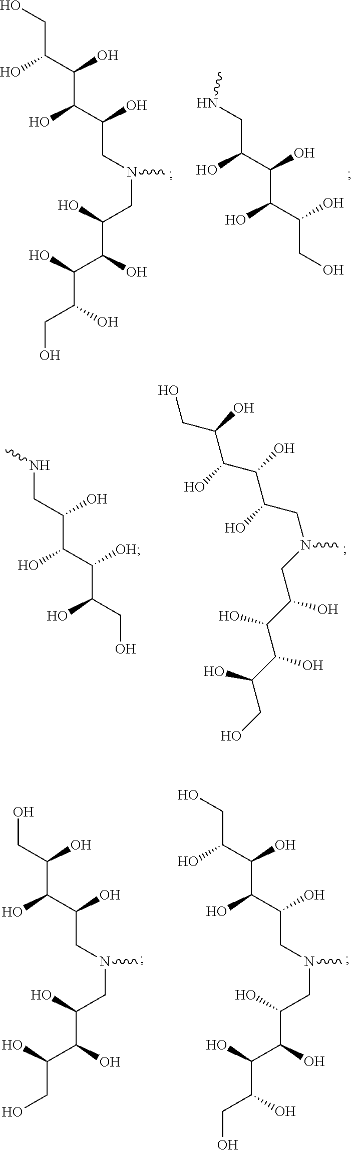

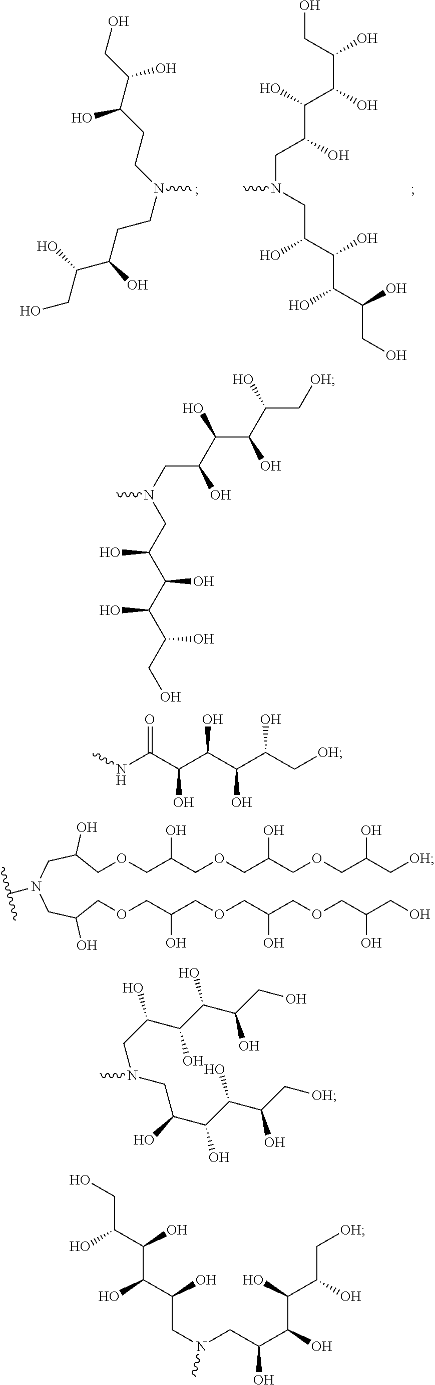

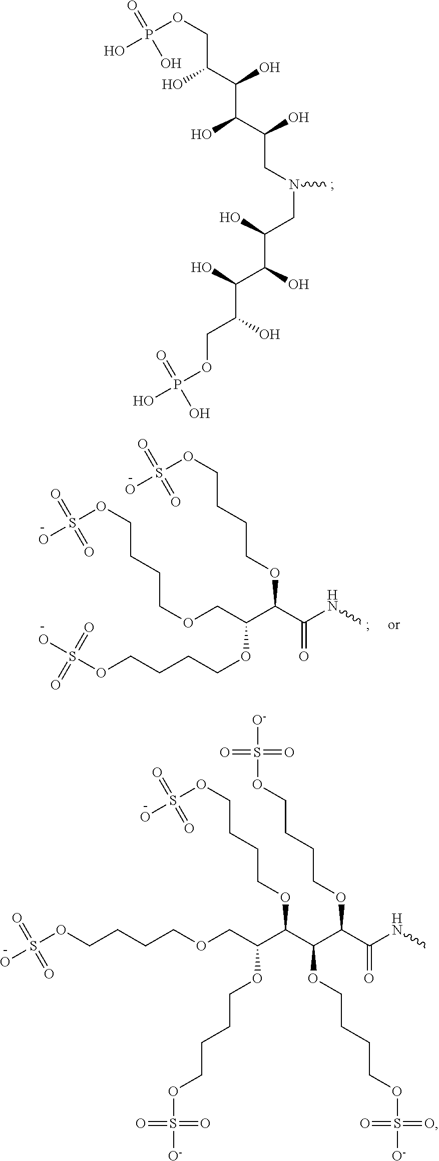

or a stereoisomer thereof, wherein the ( [symbol] ) indicates the attachment site of —NR⁴⁴R⁴⁵ to the remainder of the polar group.

Embodiment 23. The linker compound of any of Embodiments 1-22, comprising a polar group having one of the following structures prior to attachment to the linker unit:

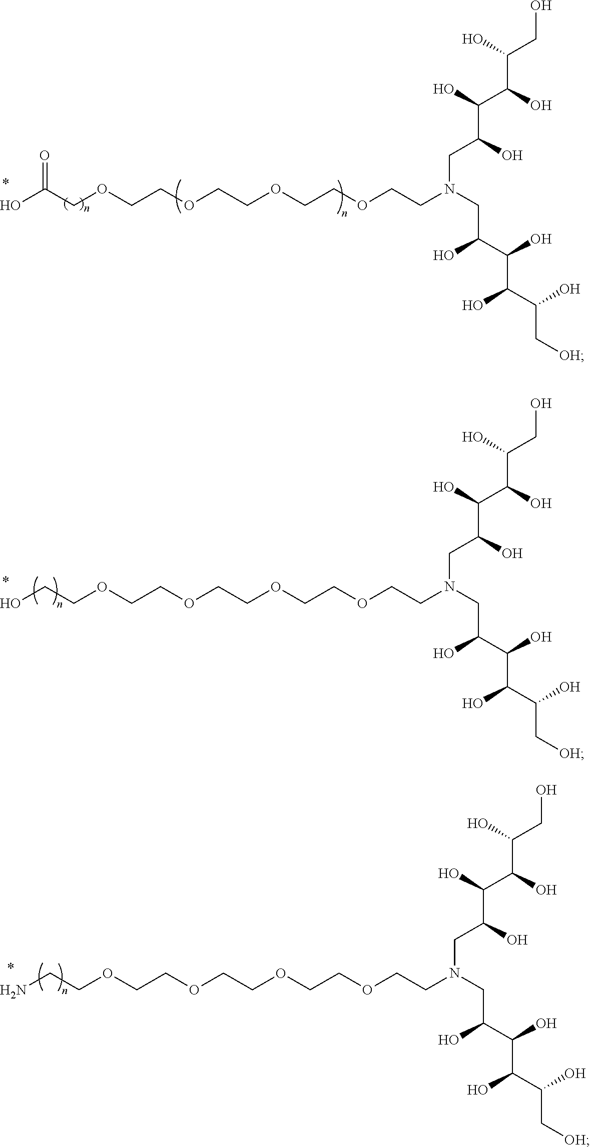

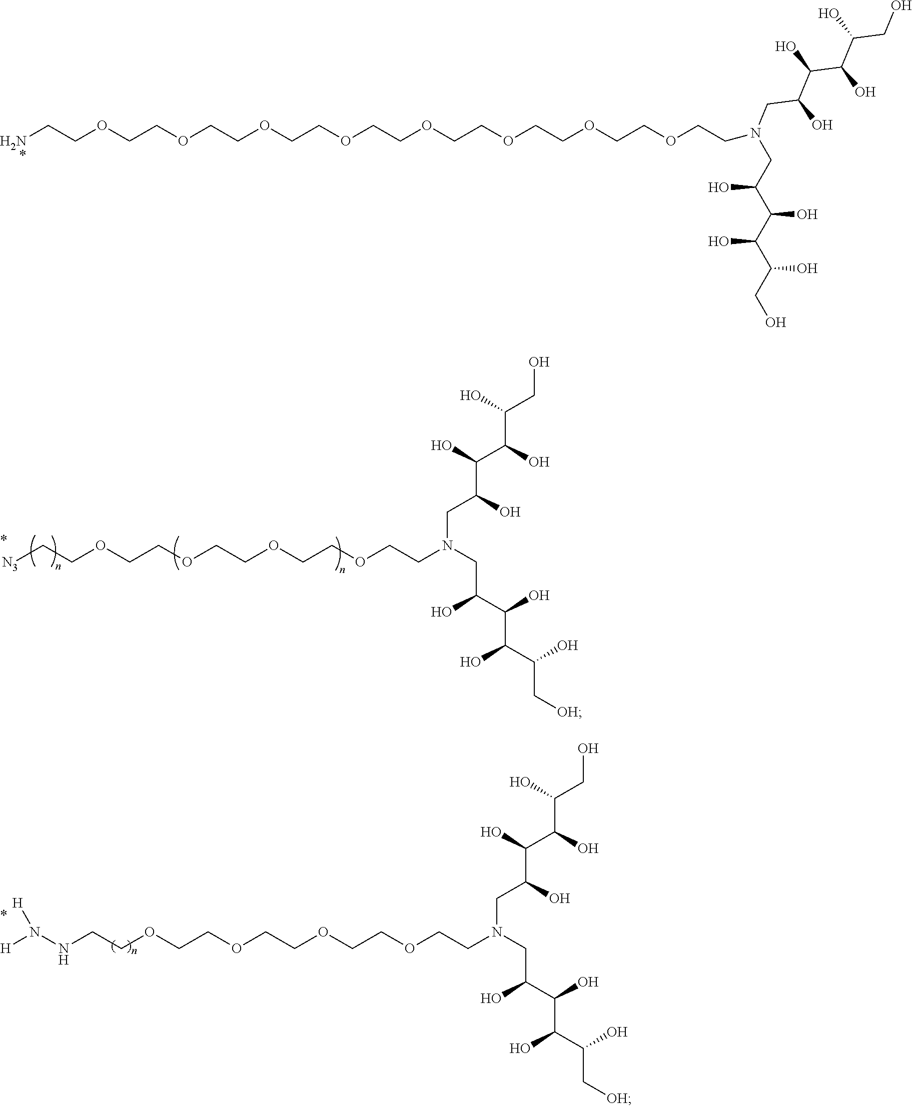

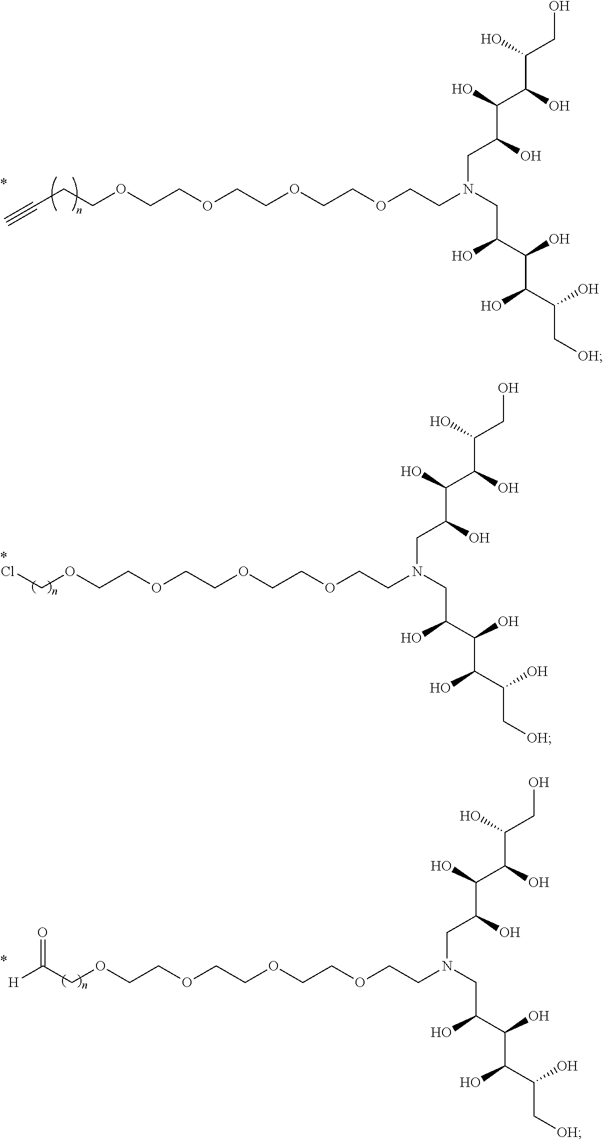

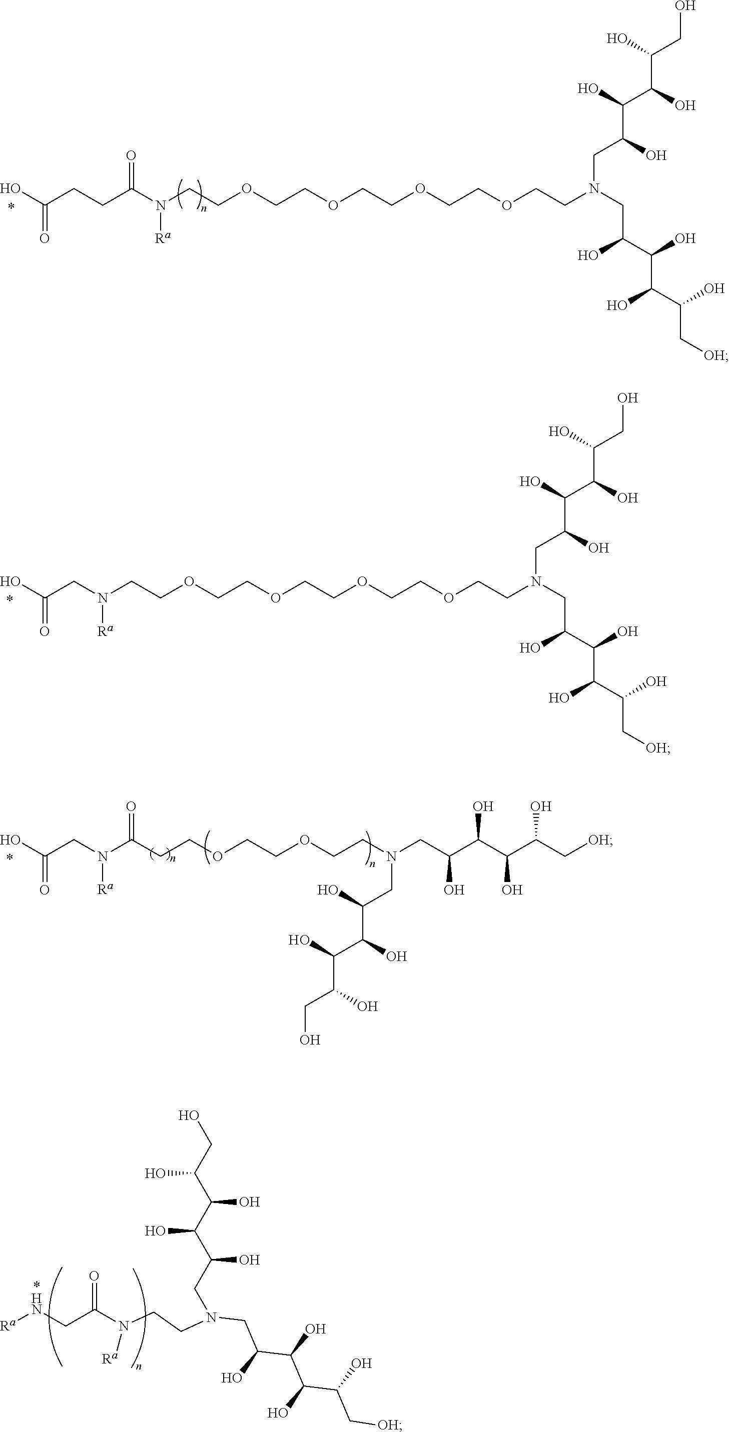

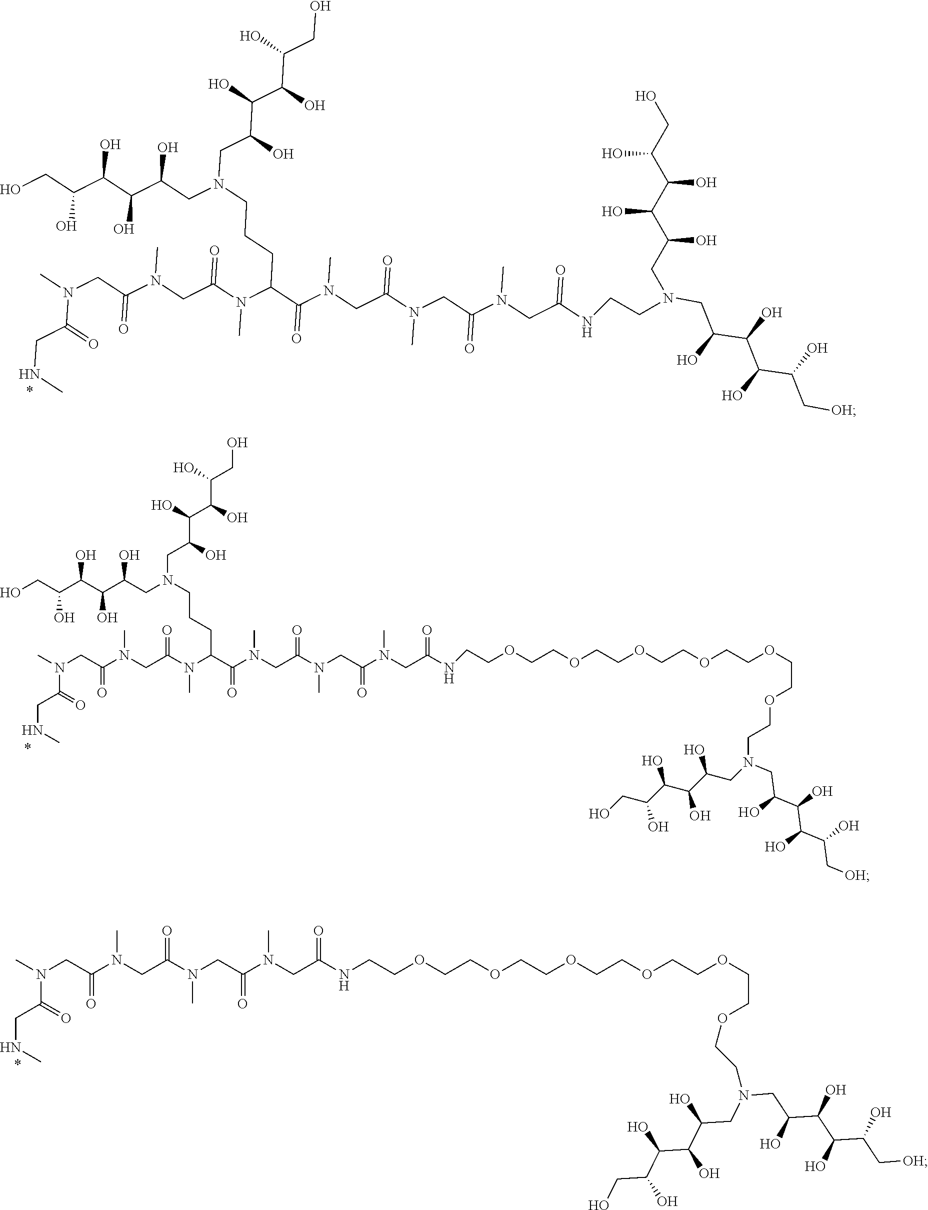

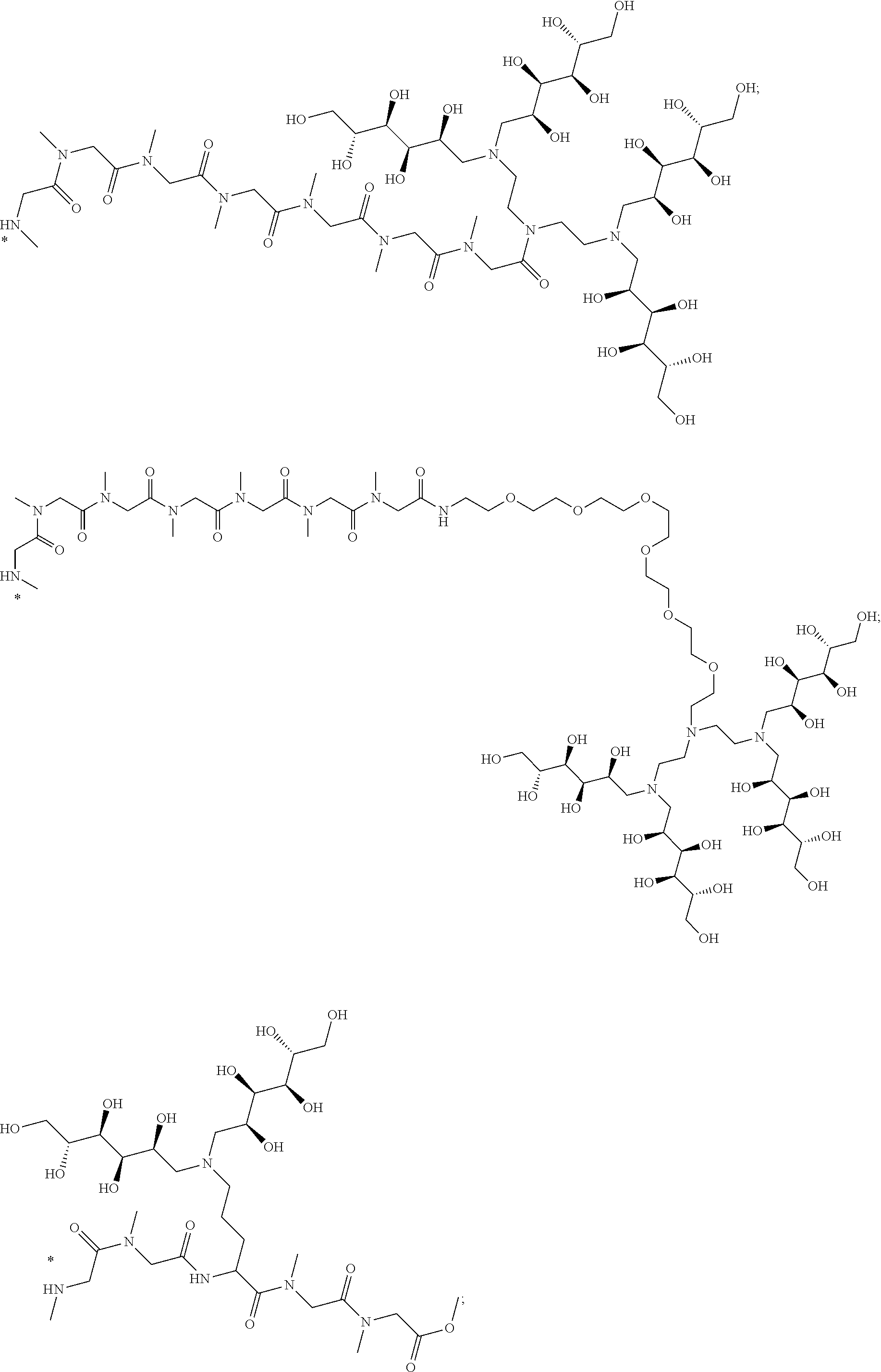

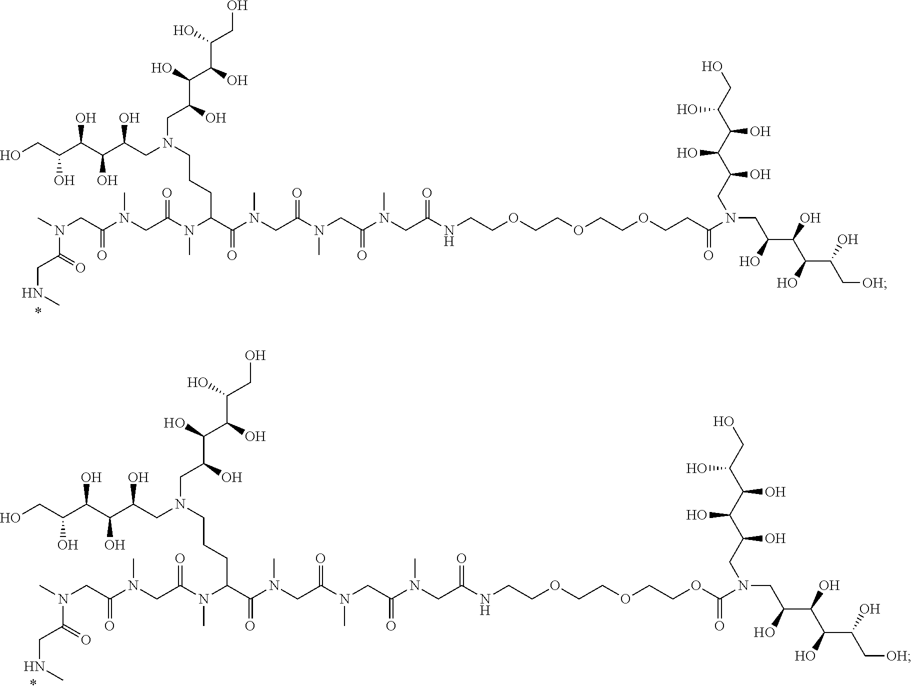

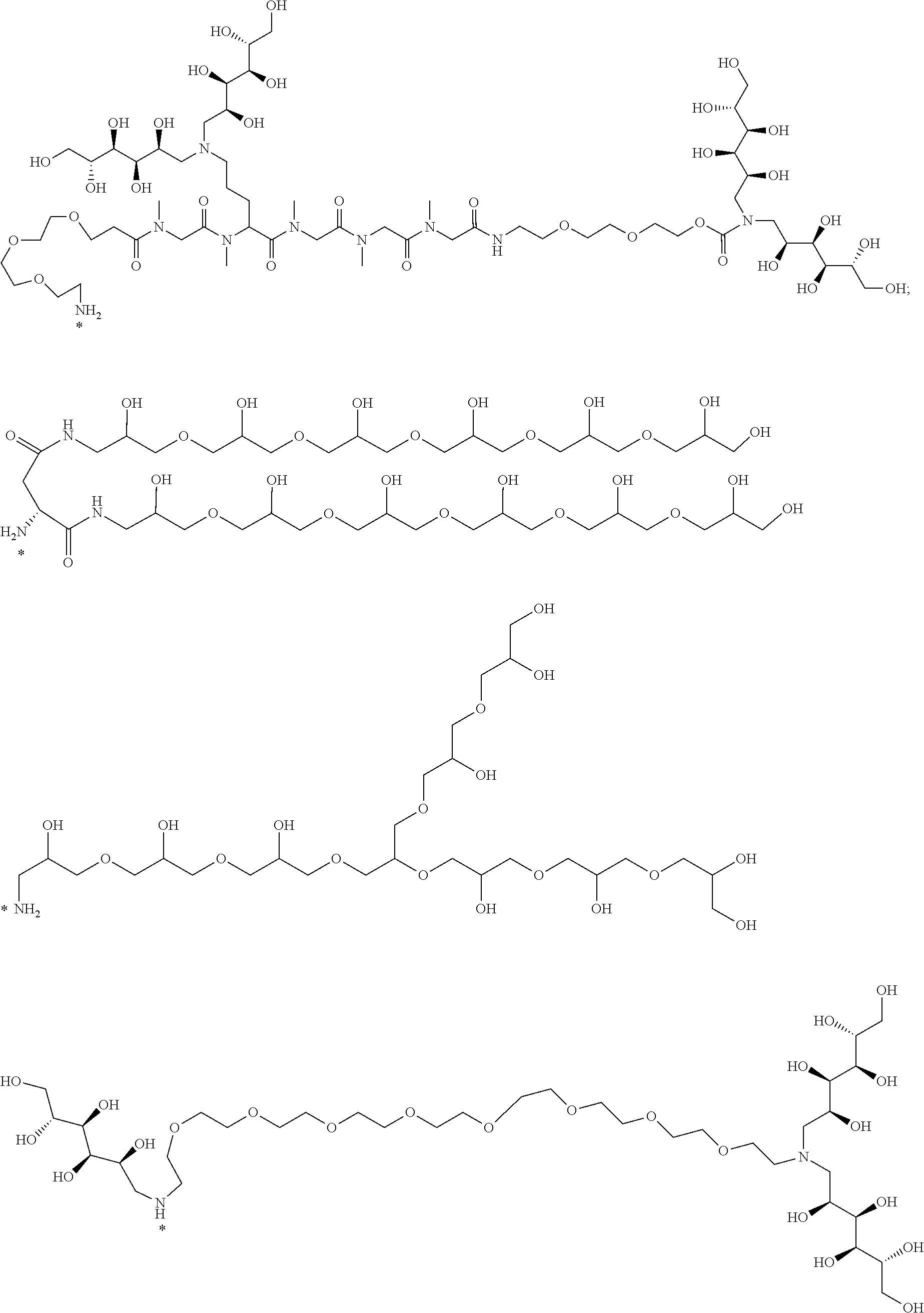

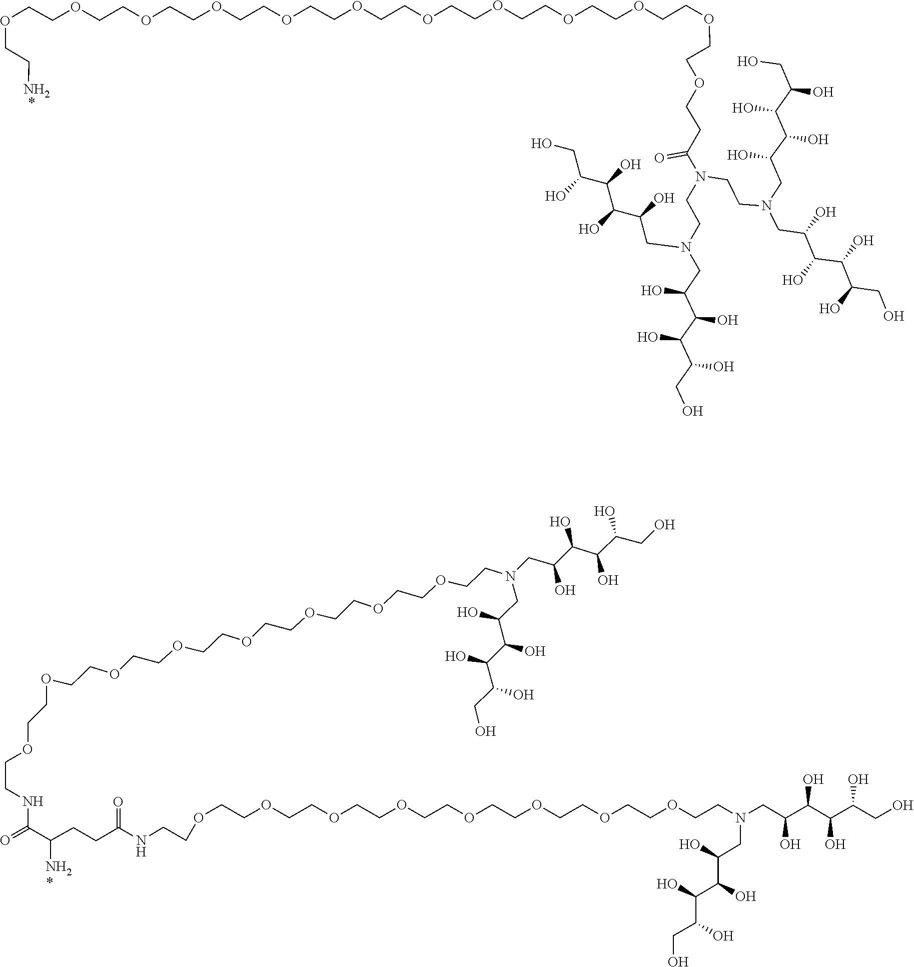

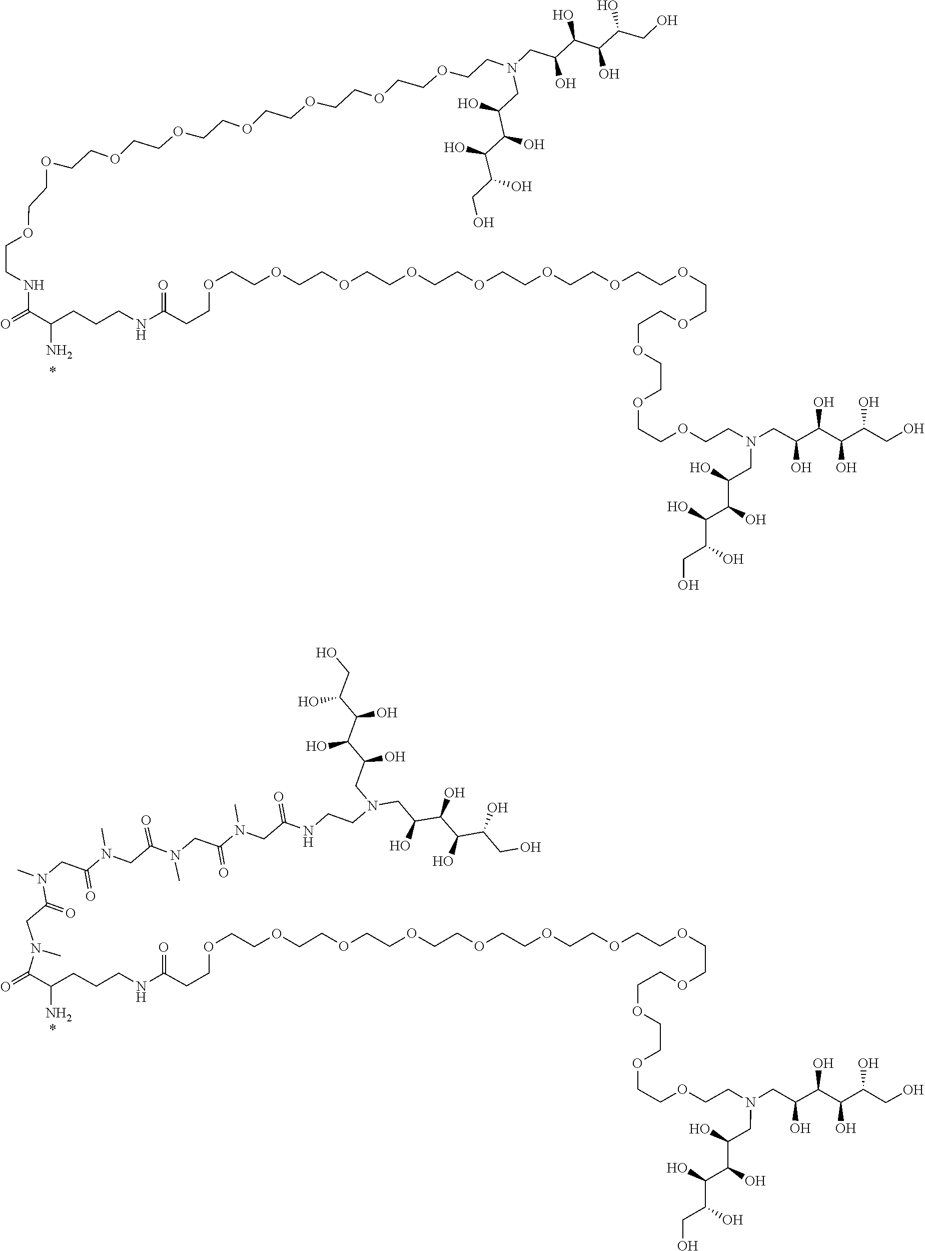

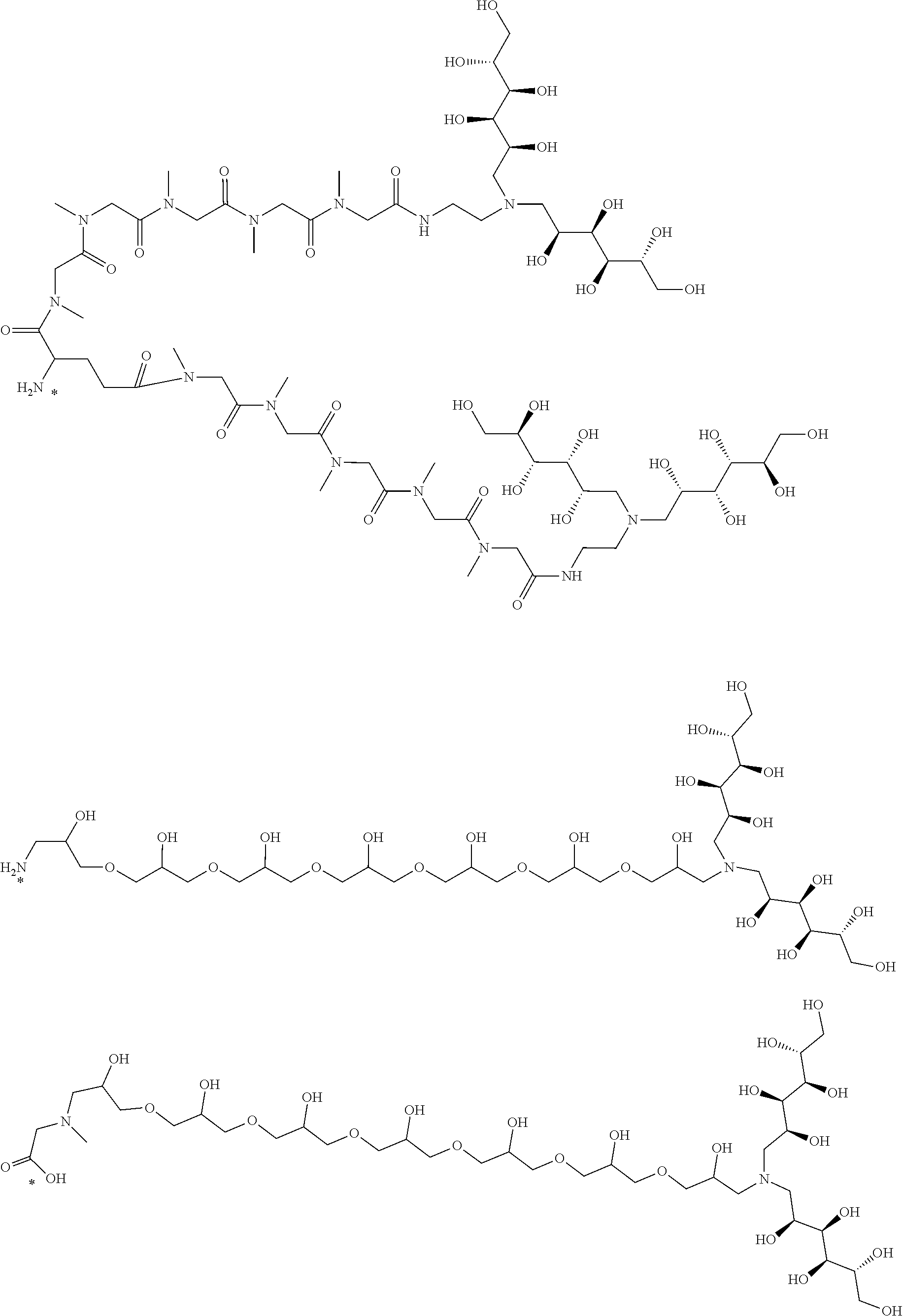

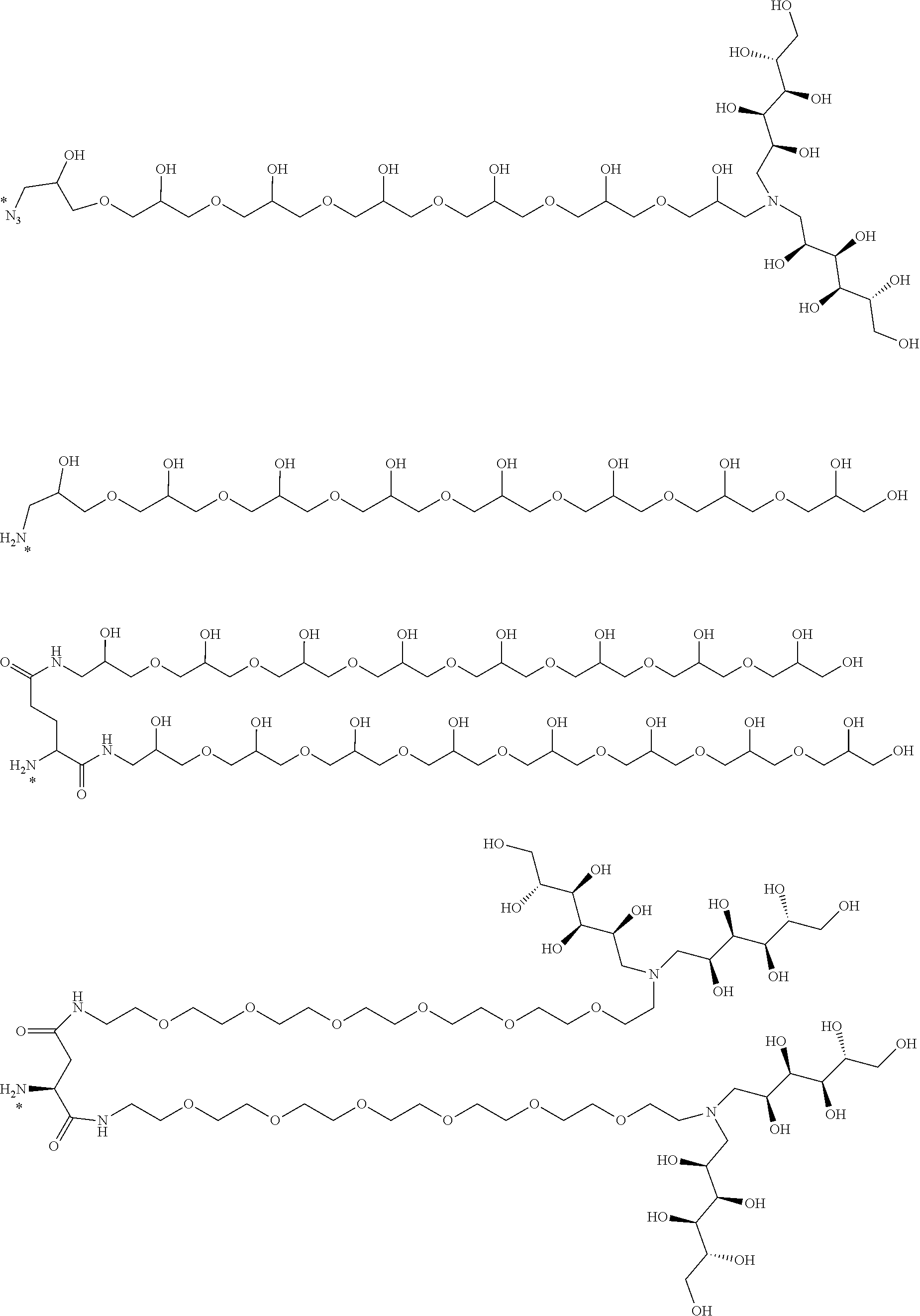

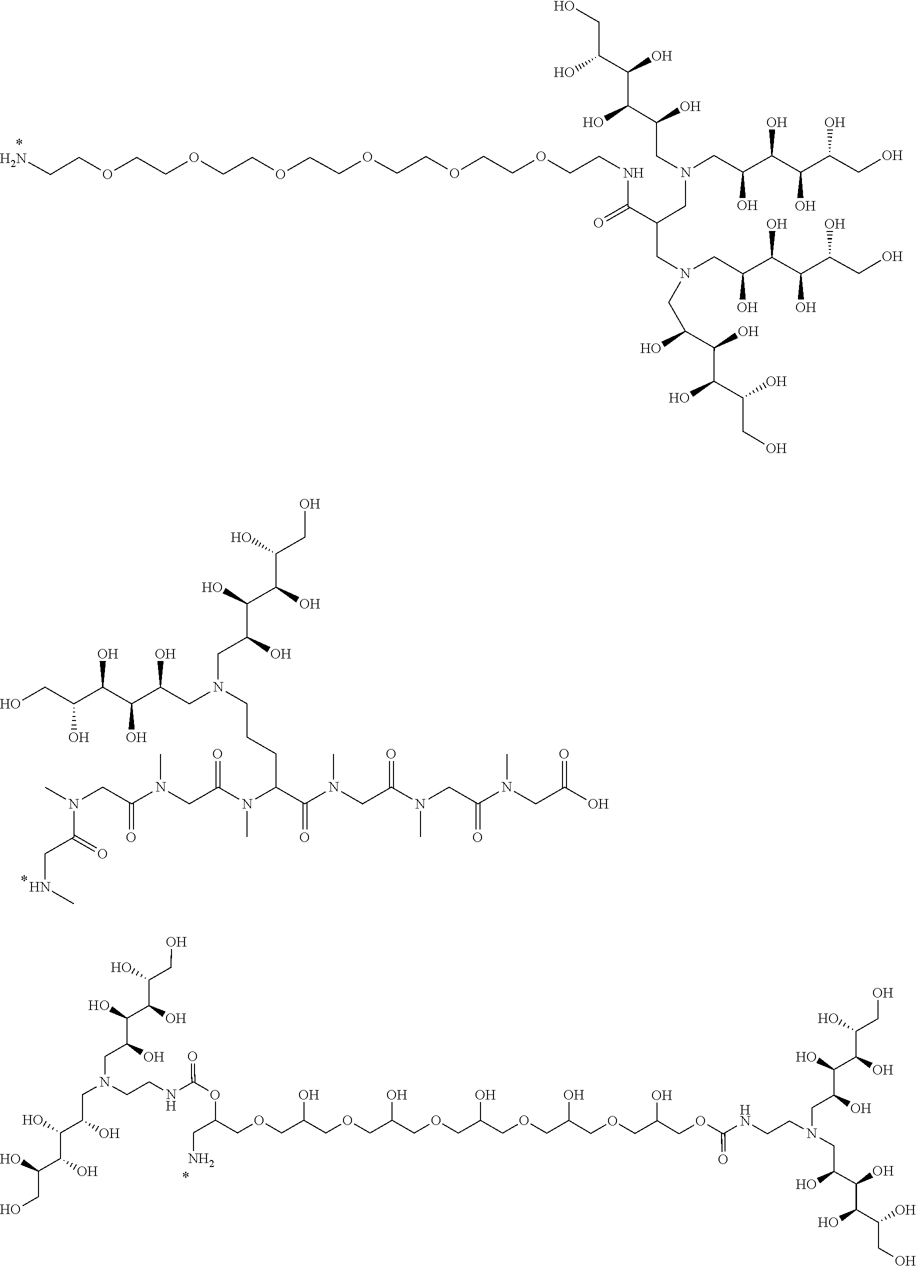

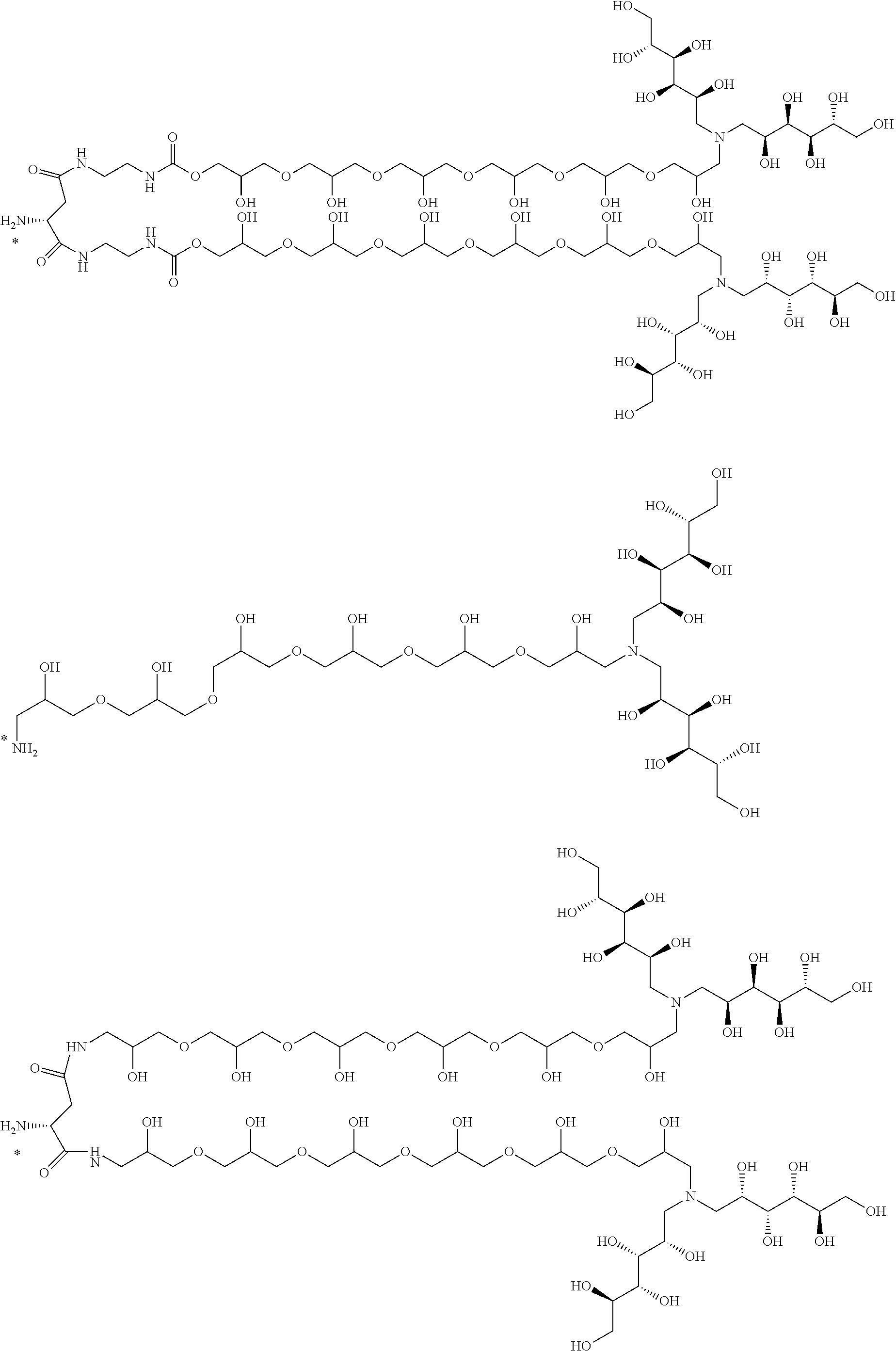

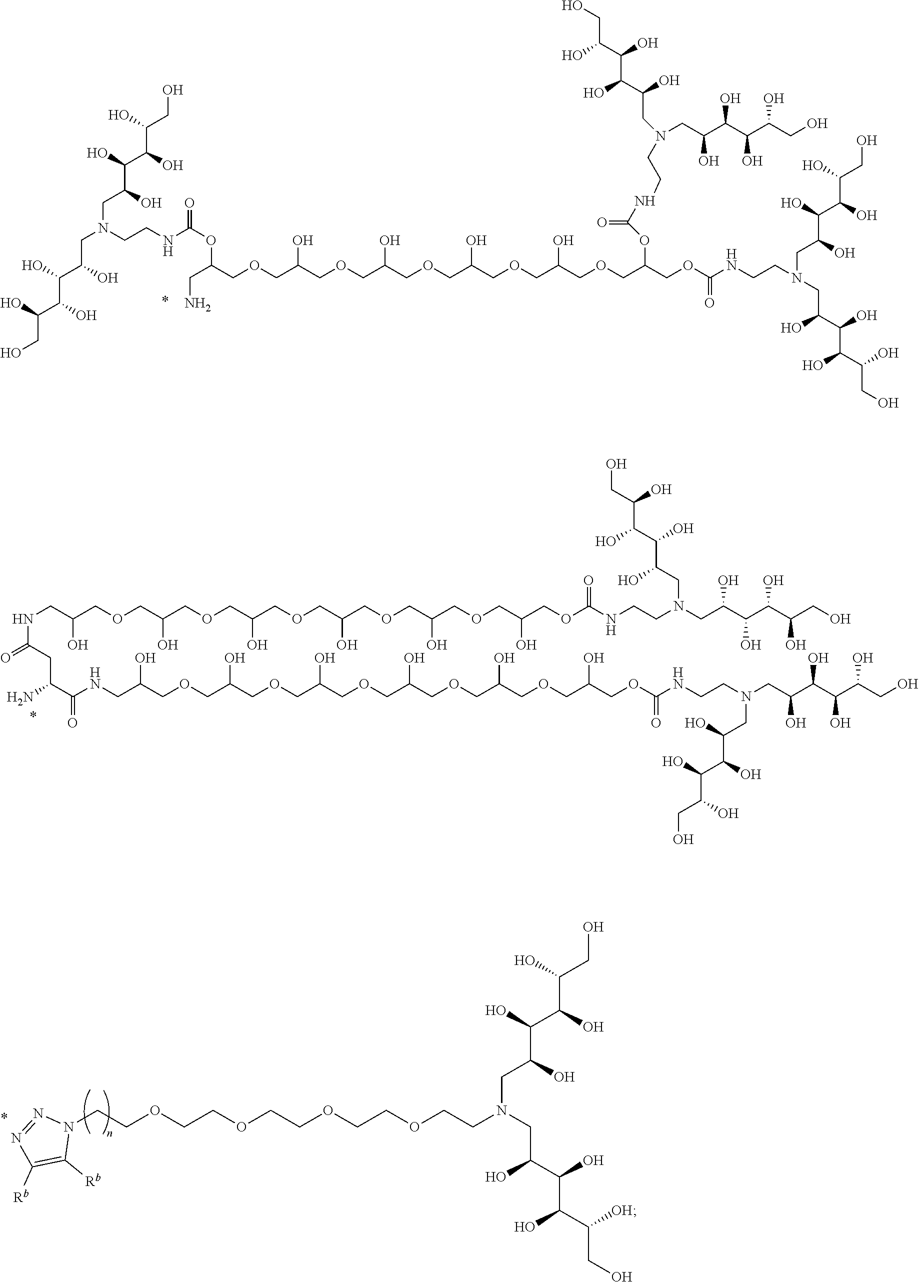

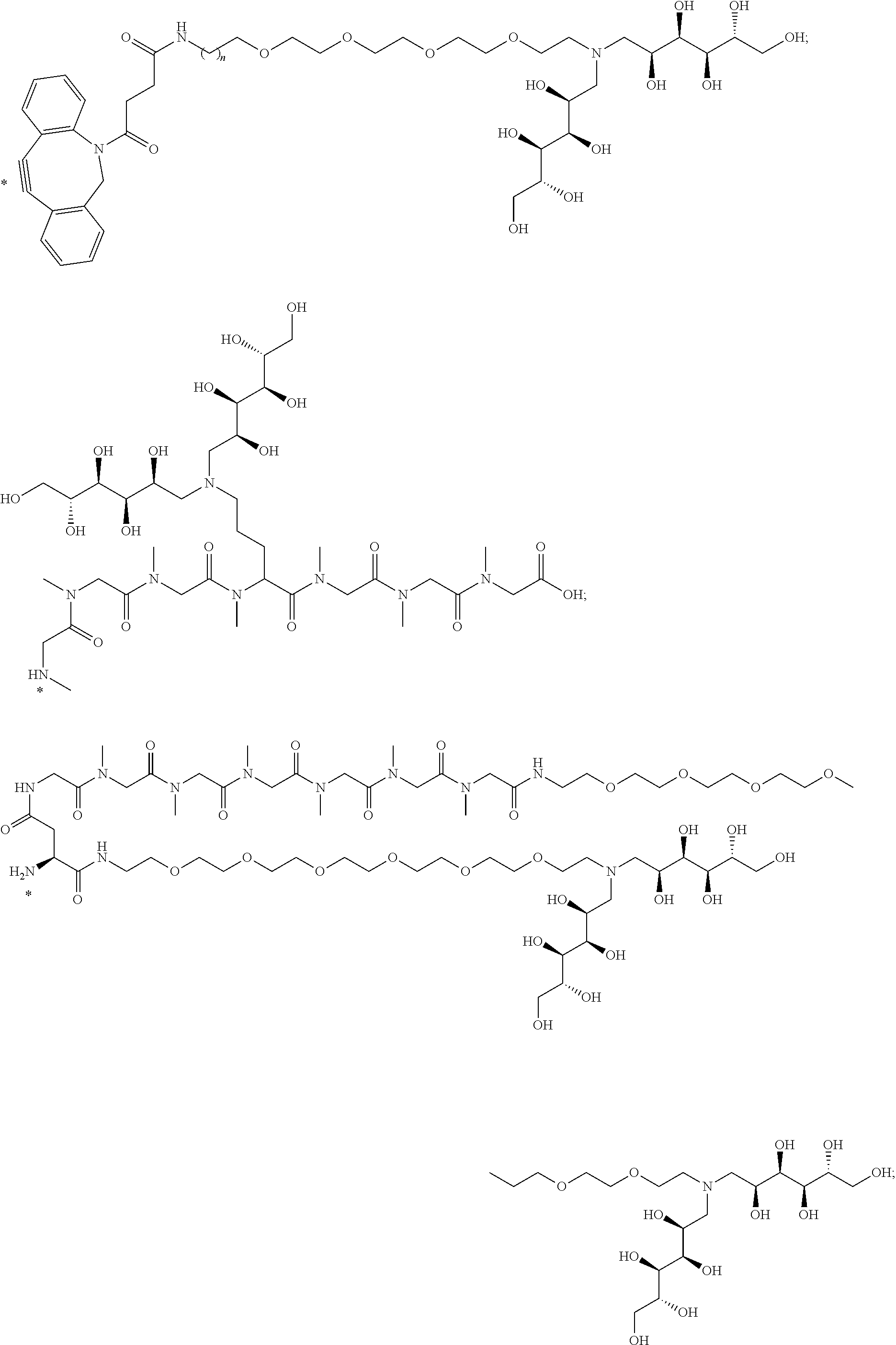

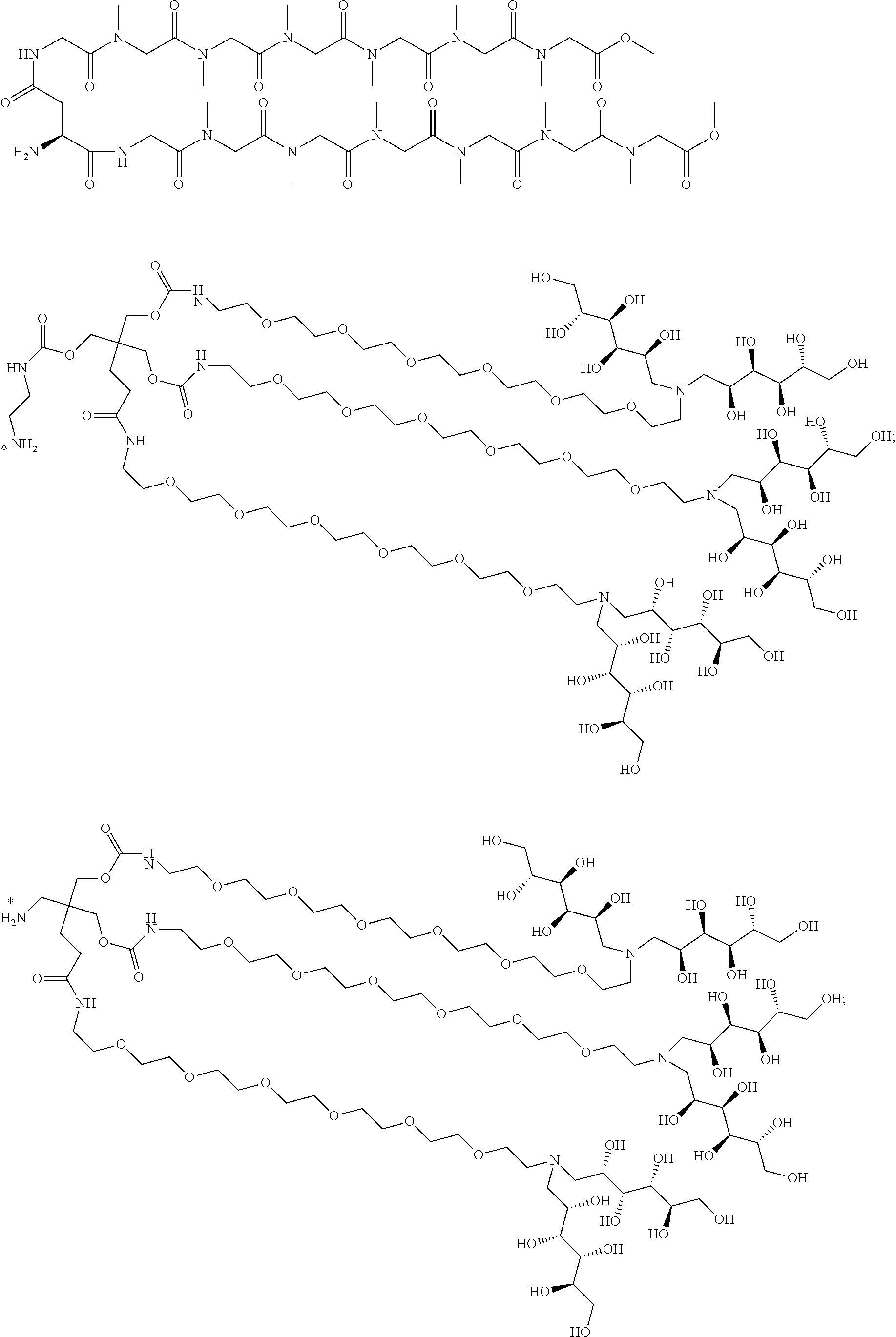

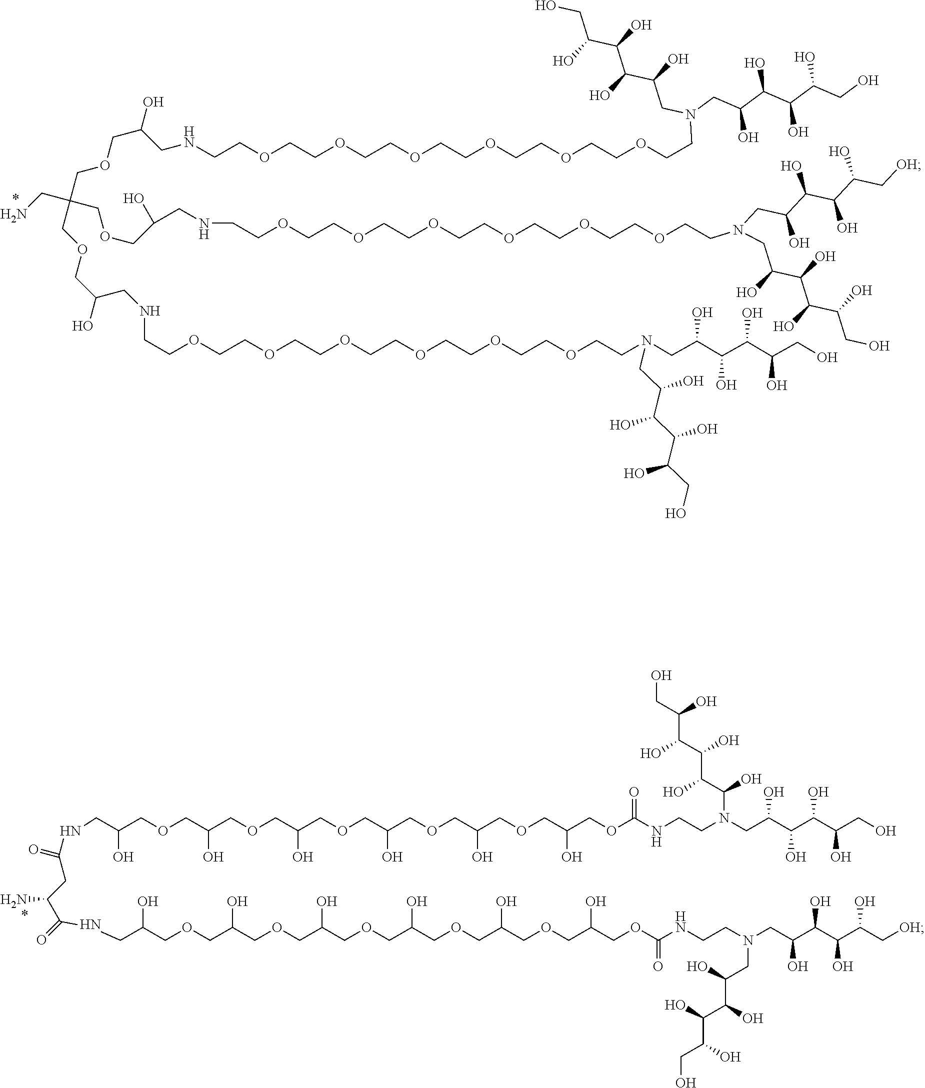

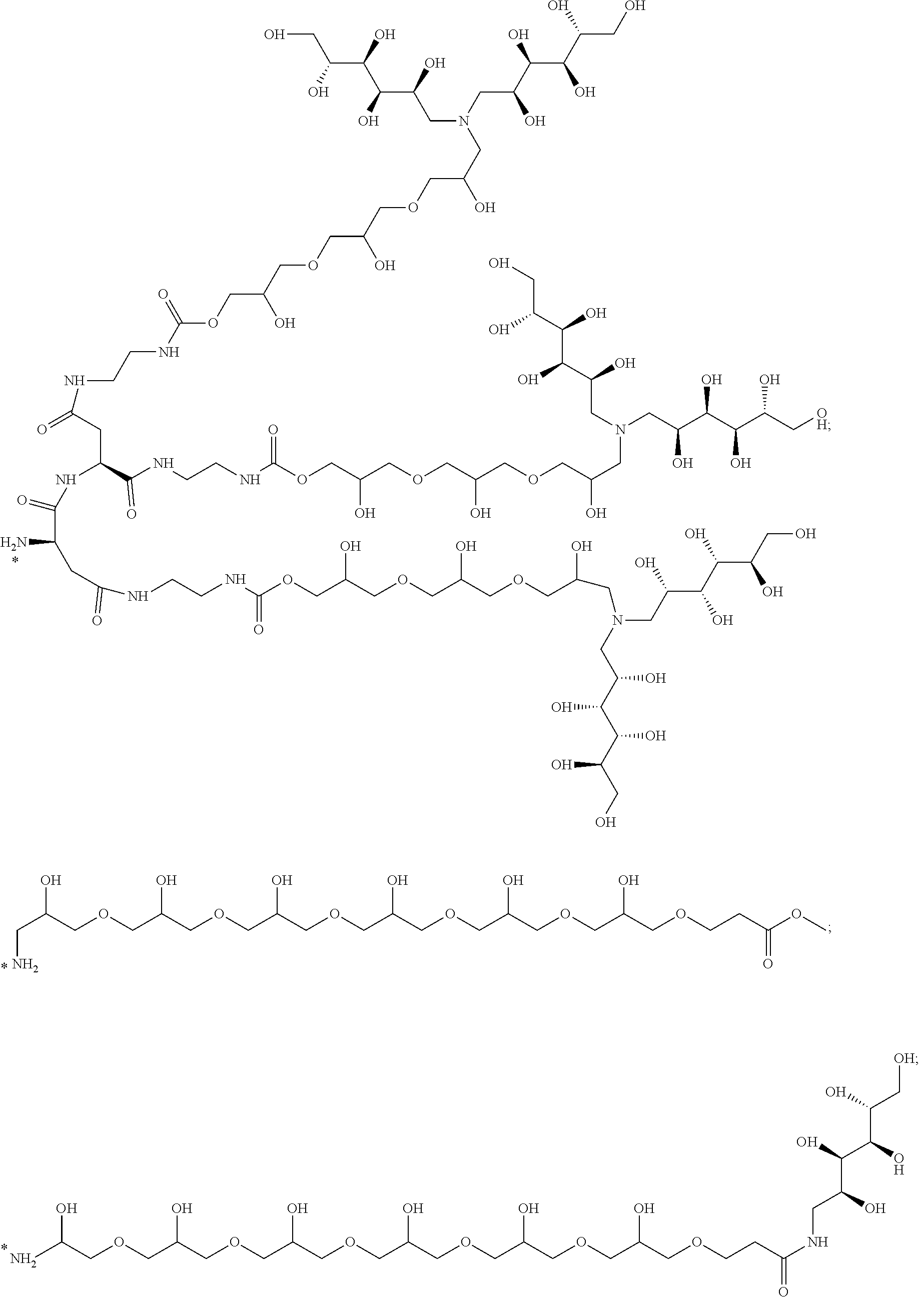

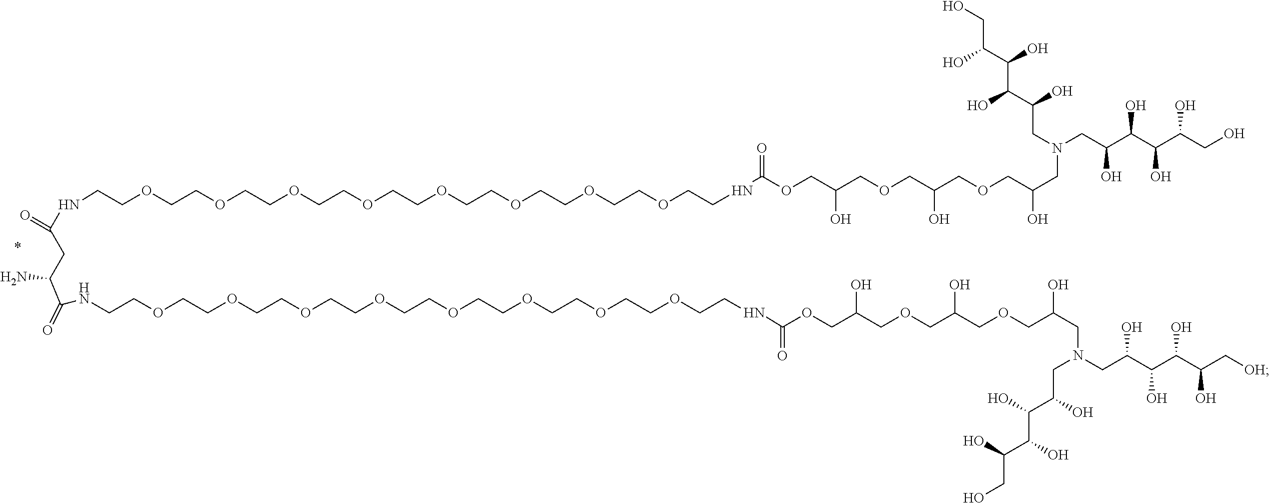

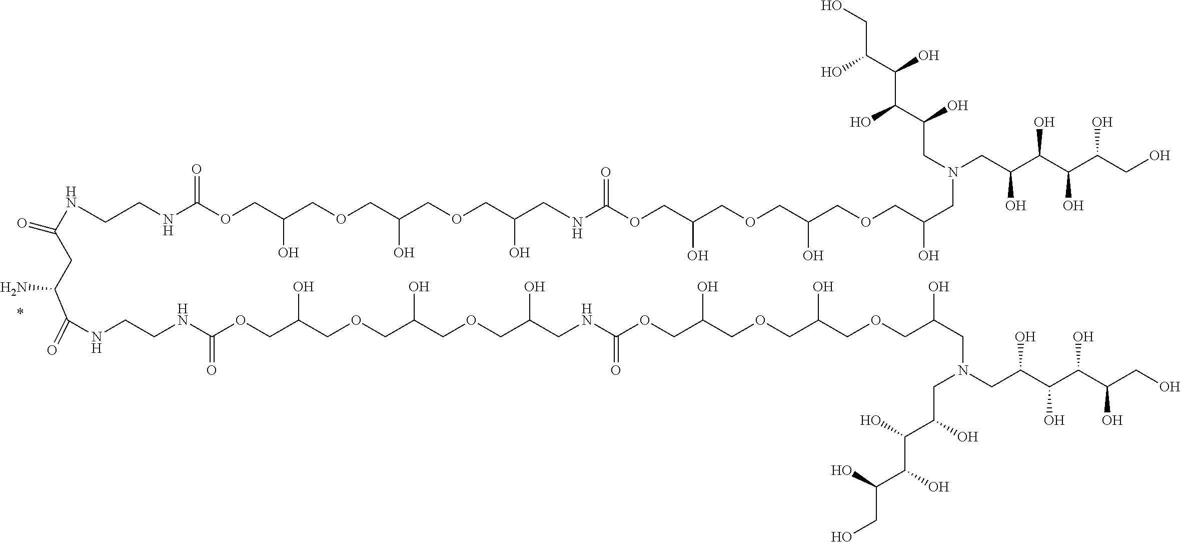

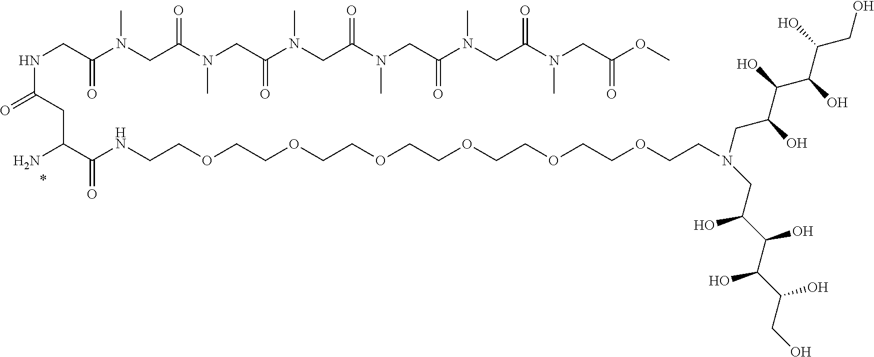

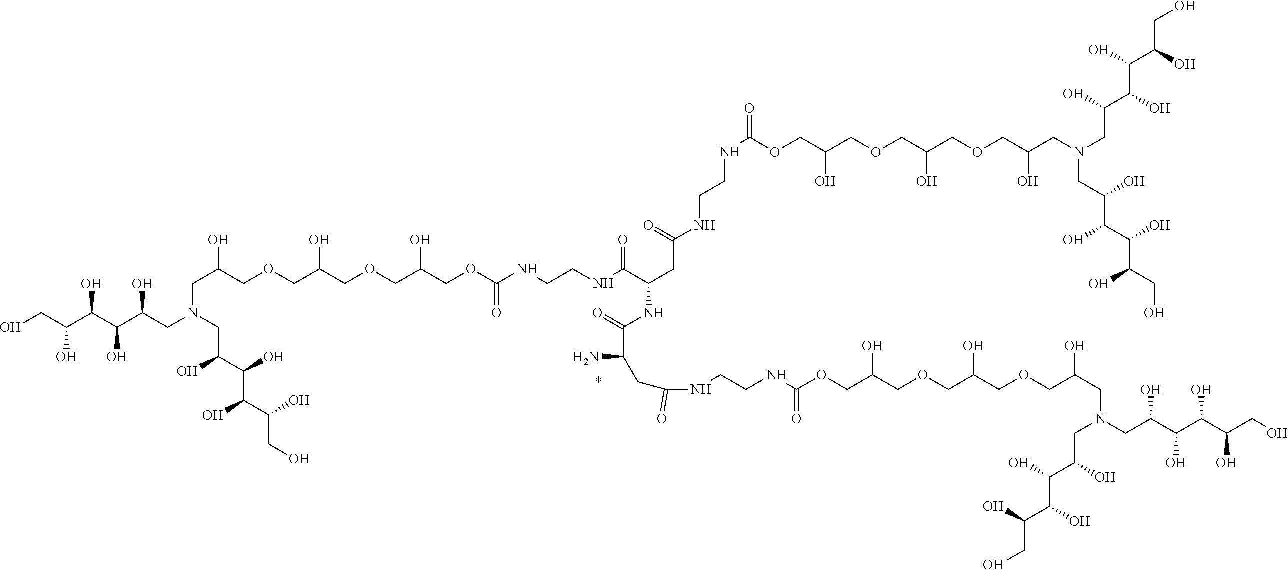

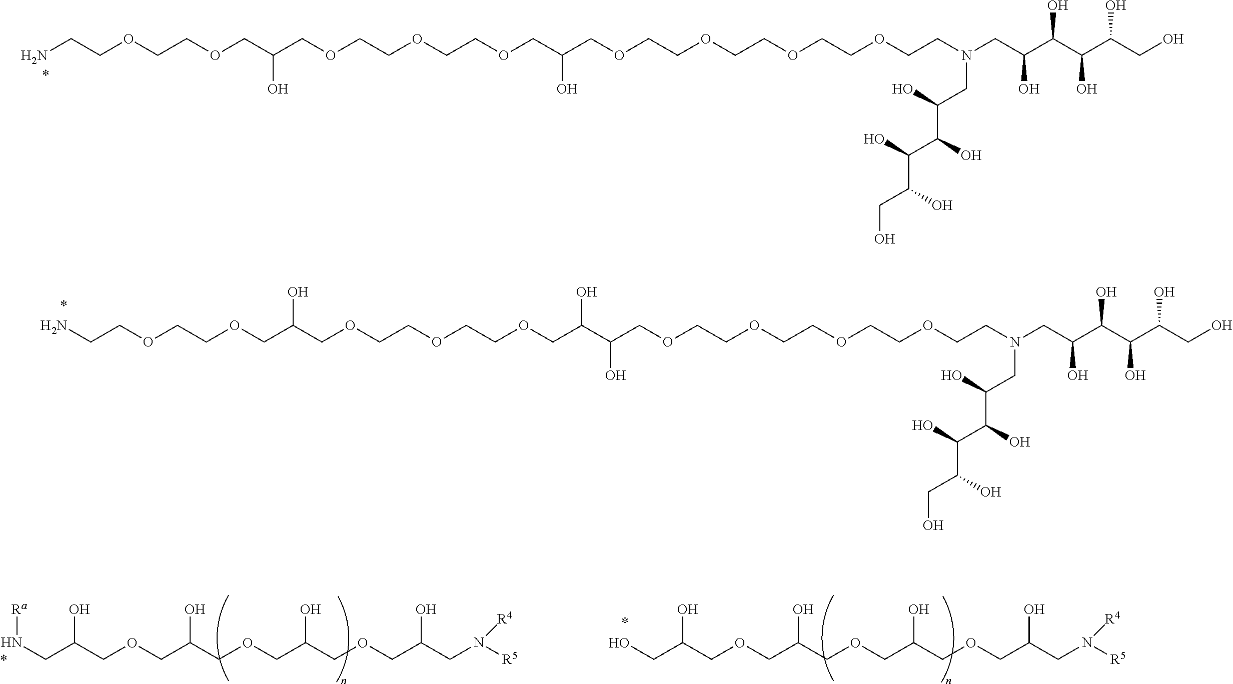

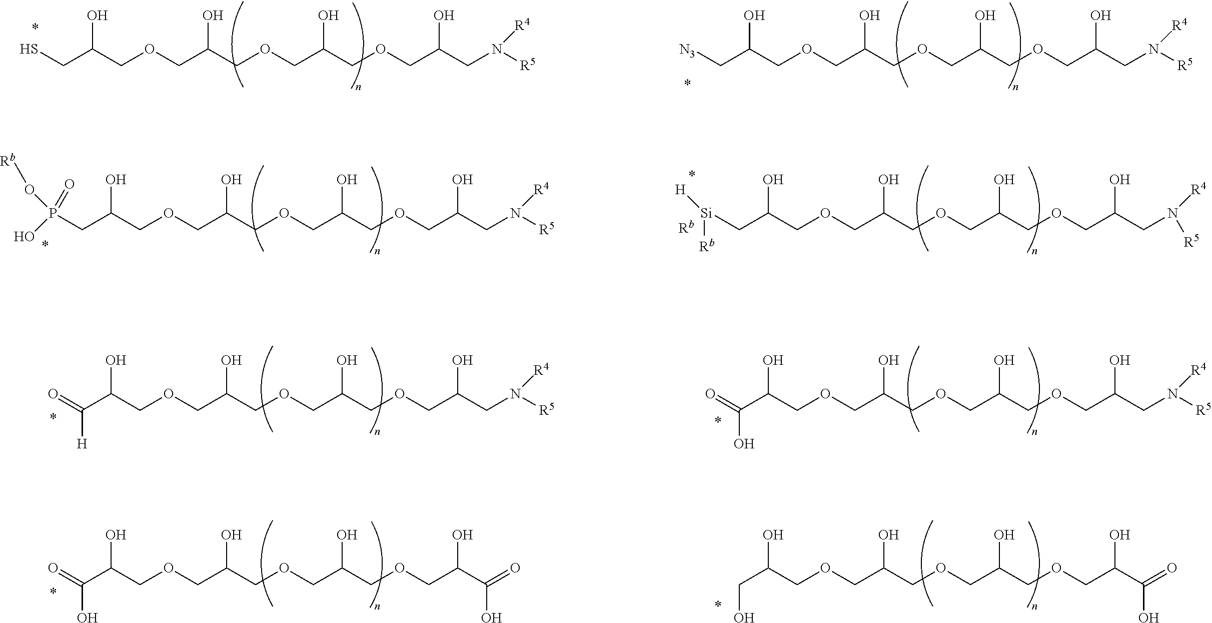

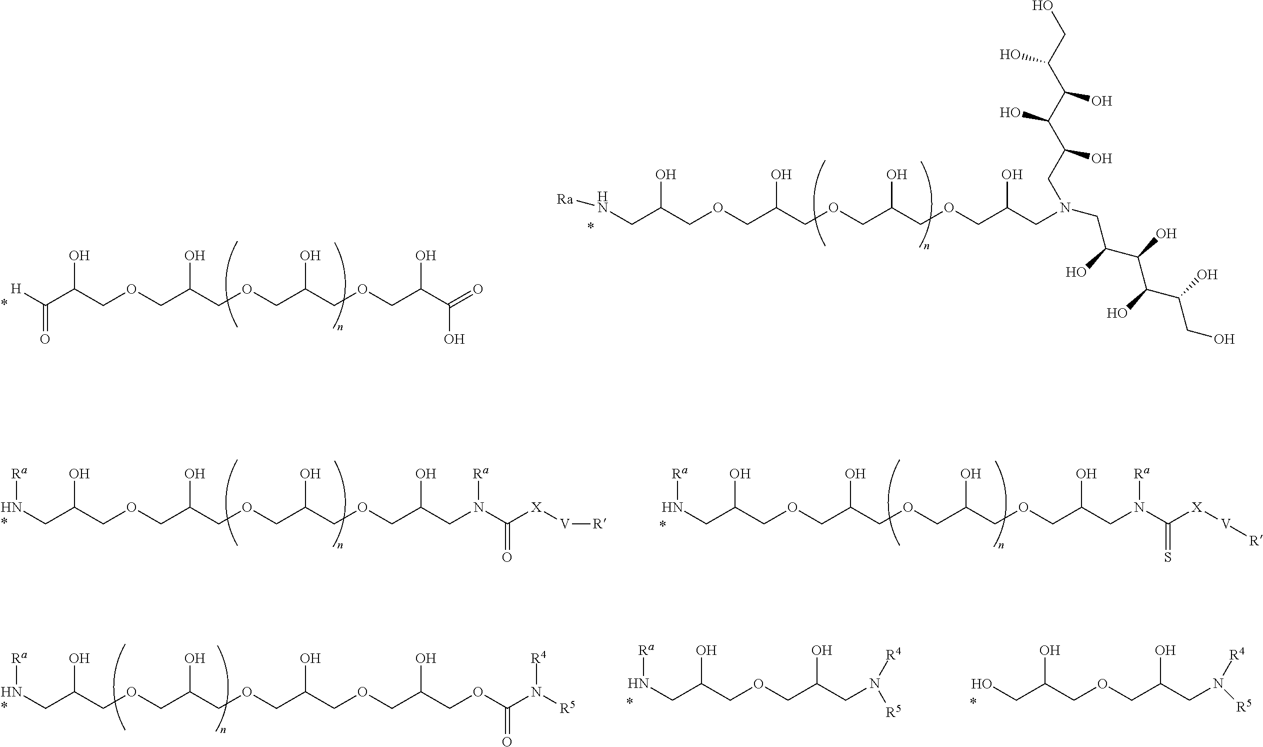

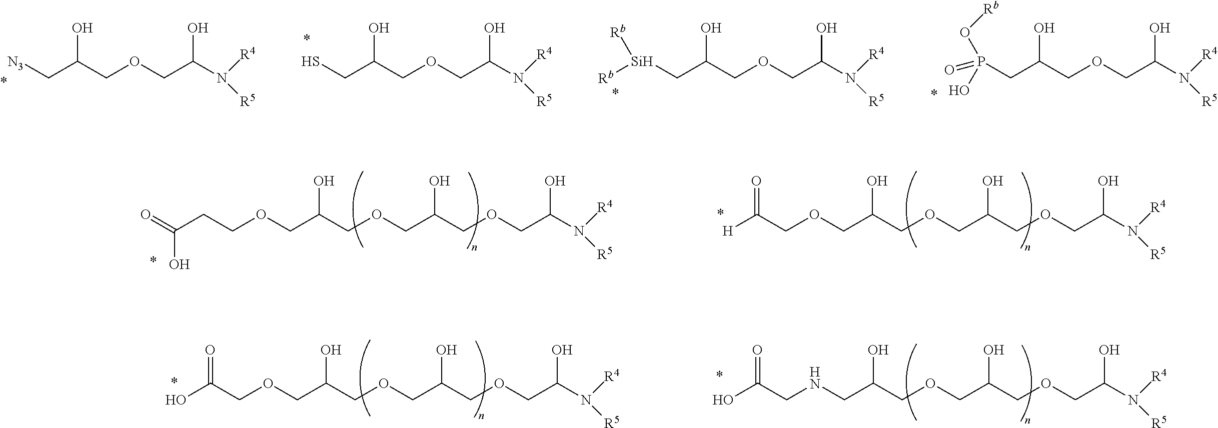

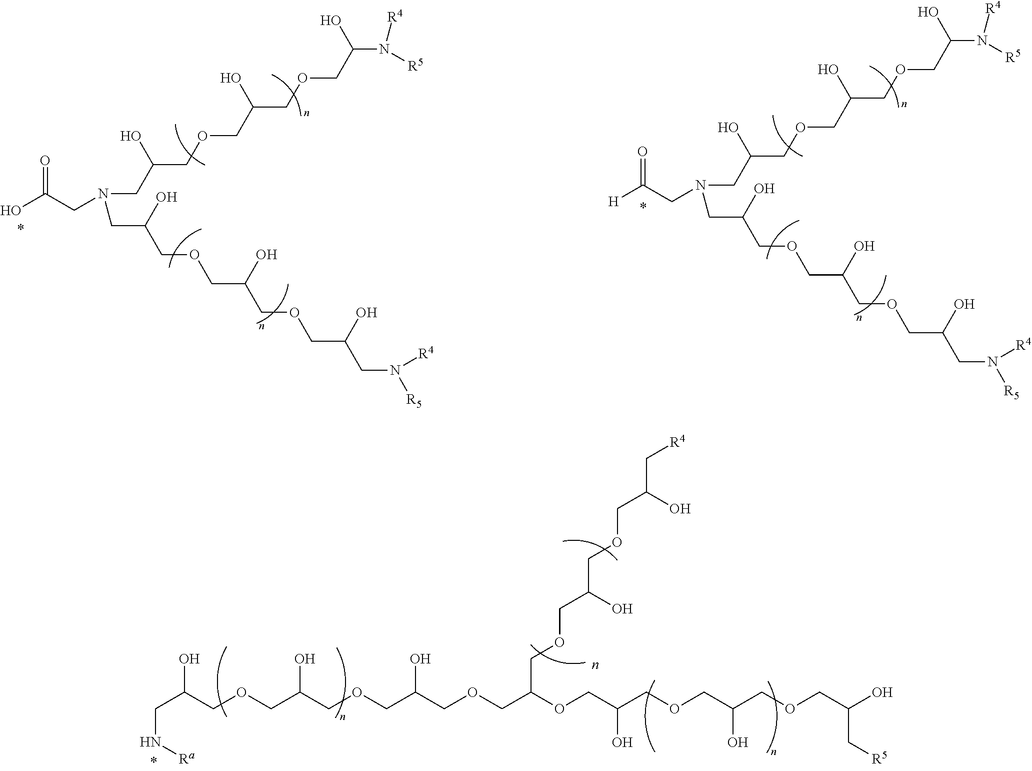

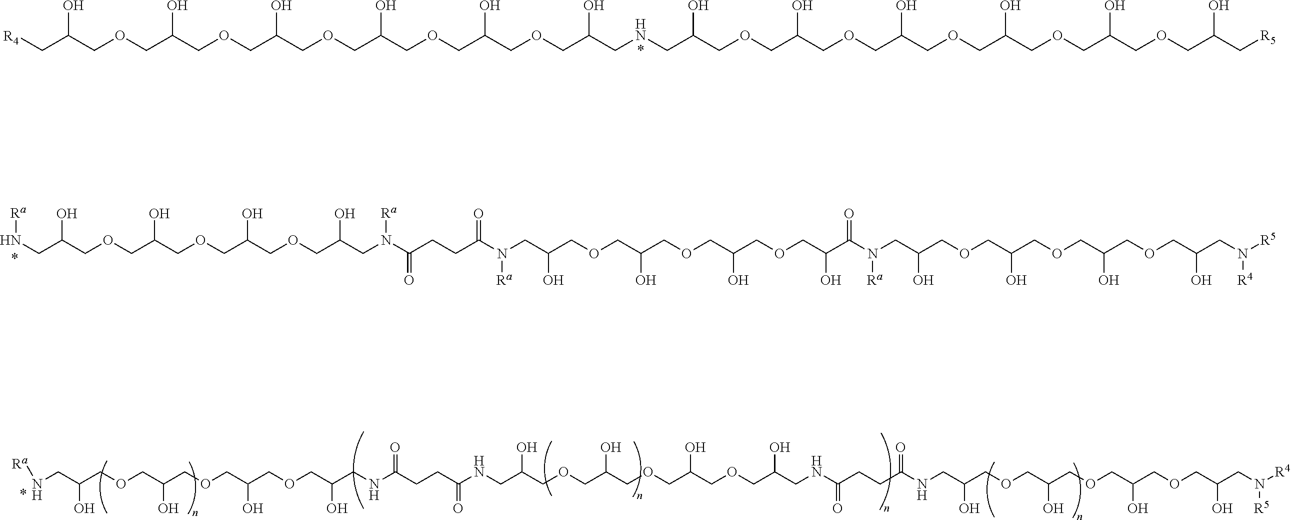

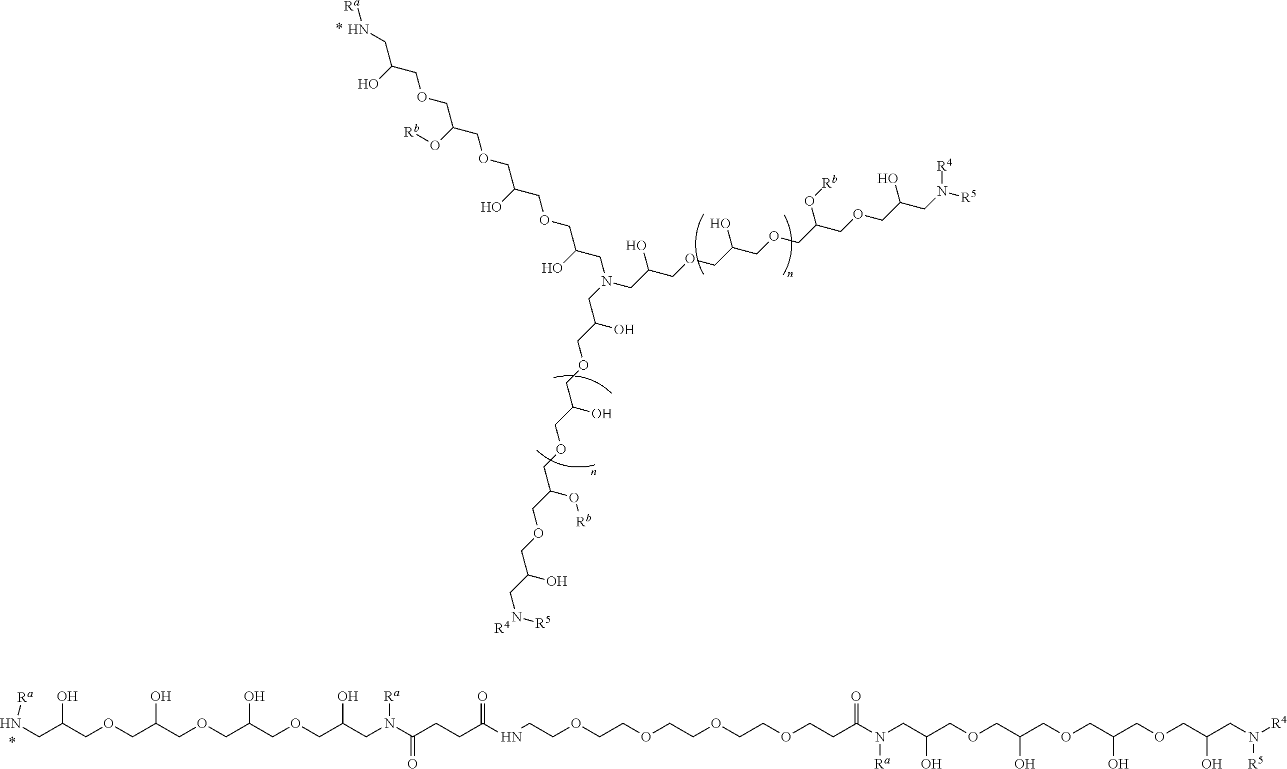

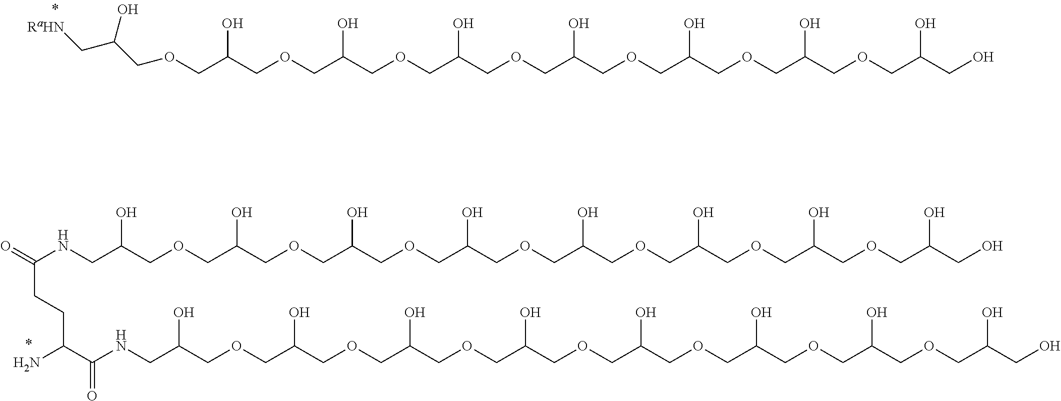

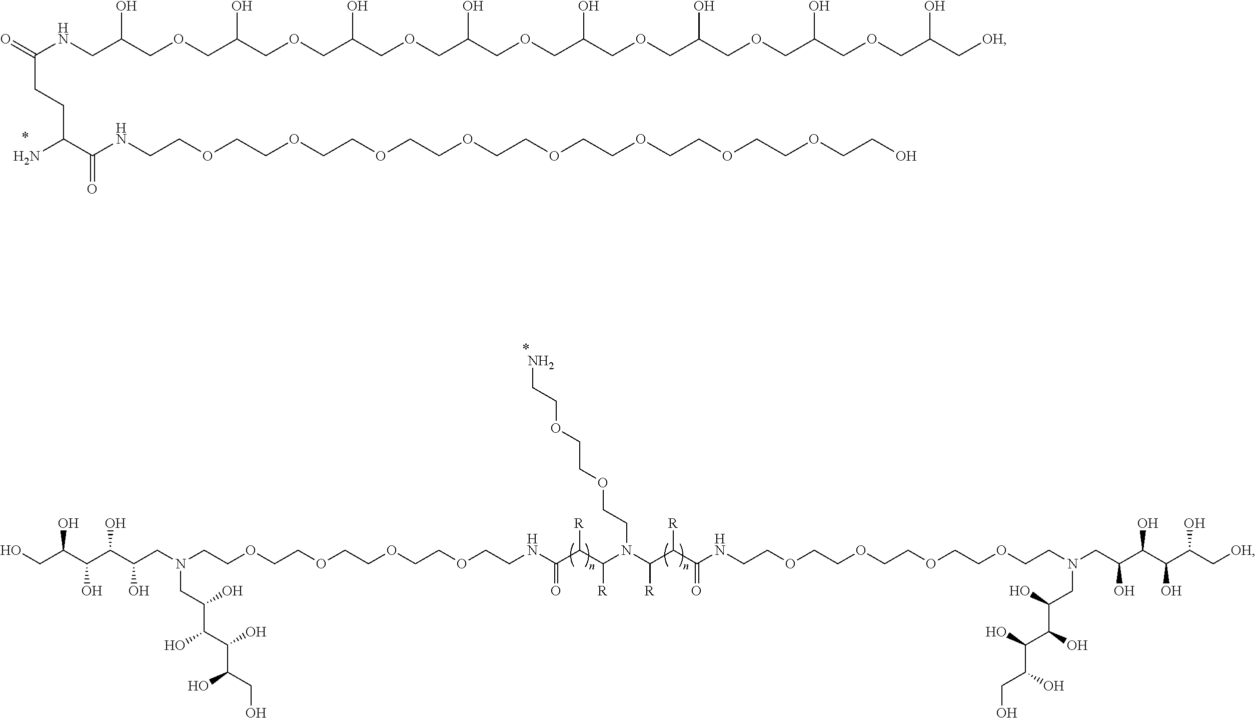

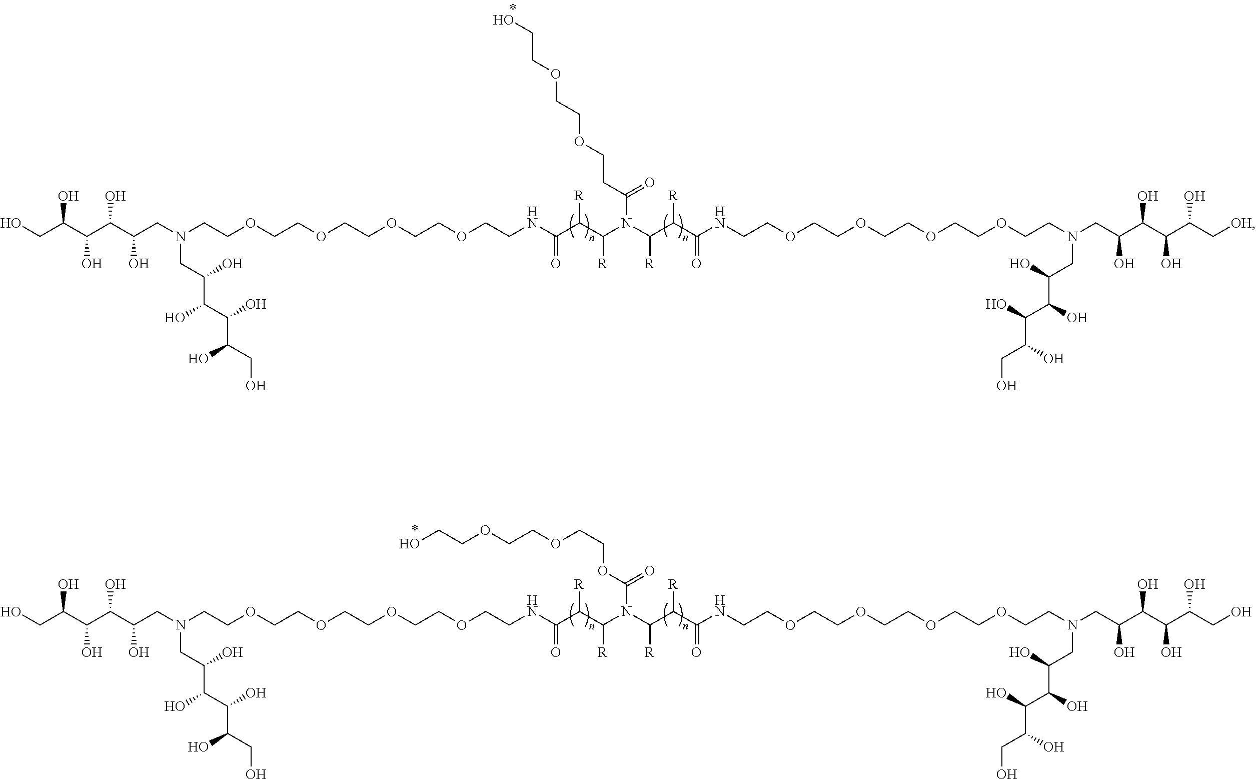

wherein:
(*) indicates the attachment site β or to site R^{b}, or to the enzyme-cleavable group;
each R is independently H or C₁-C₆ alkyl;
R′ is H, C₁-C₆ alkyl, —N(R²⁴)(R²⁵) or —CO₂H;
each n is independently 1 to 12;
X is O, NR or —CH₂—;
V is bond or C₁-C₆ alkyl;
one of R²⁴ and R²⁵ is selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a sugar unit of formula (XII) or (XIII); and the other of R²⁴ and R²⁵ is selected from H; polyhydroxyl group; substituted polyhydroxyl group; —C(O)— polyhydroxyl group; substituted —C(O)-polyhydroxyl group; substituted —C(O)—C₁-C₈ alkyl; a chelator; —C(O)—R²⁸, where R²⁸ is a sugar unit of formula (XII) or (XIII); and polyethylene glycol, optionally having 1 to 24 ethylene glycol subunits, provided that R²⁴ and R²⁵ are not both H.

Embodiment 24. The linker compound of any of Embodiments 1-23, comprising a polar group selected from the following, or a stereoisomer or salt thereof:

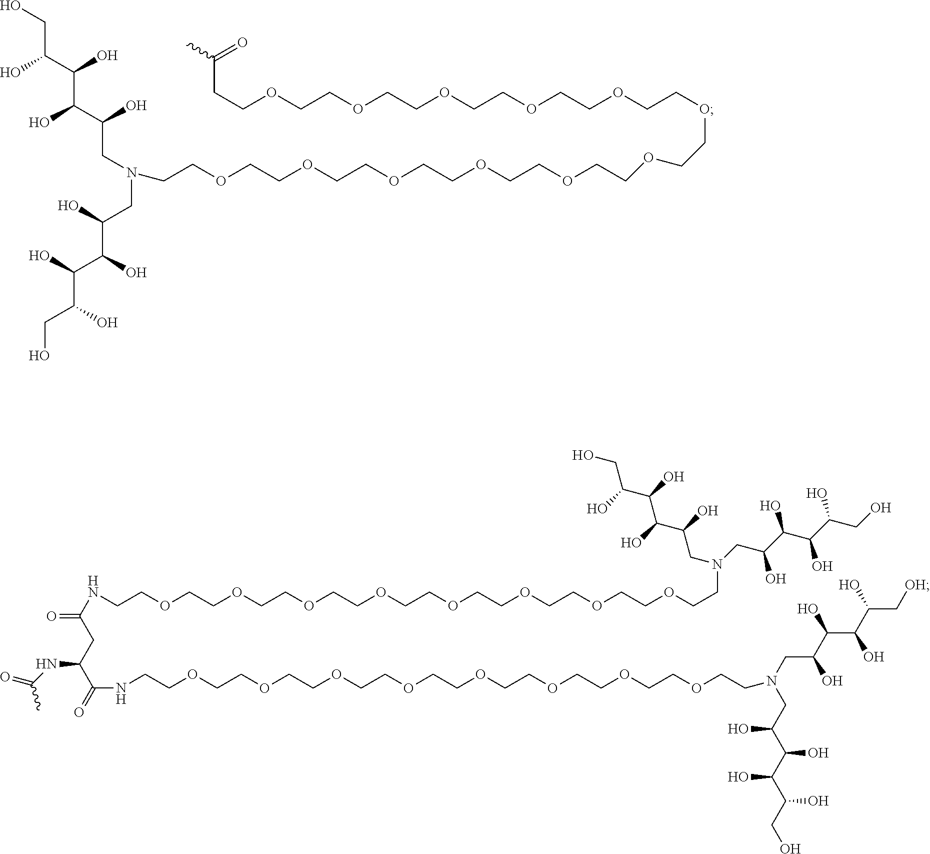

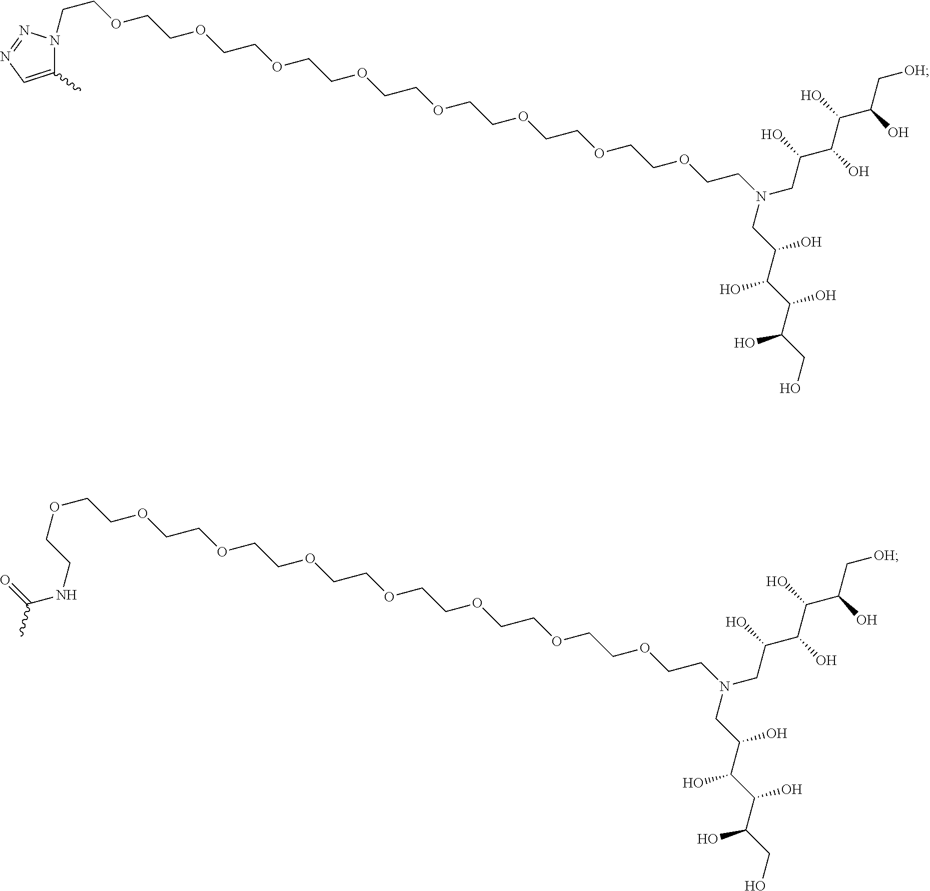

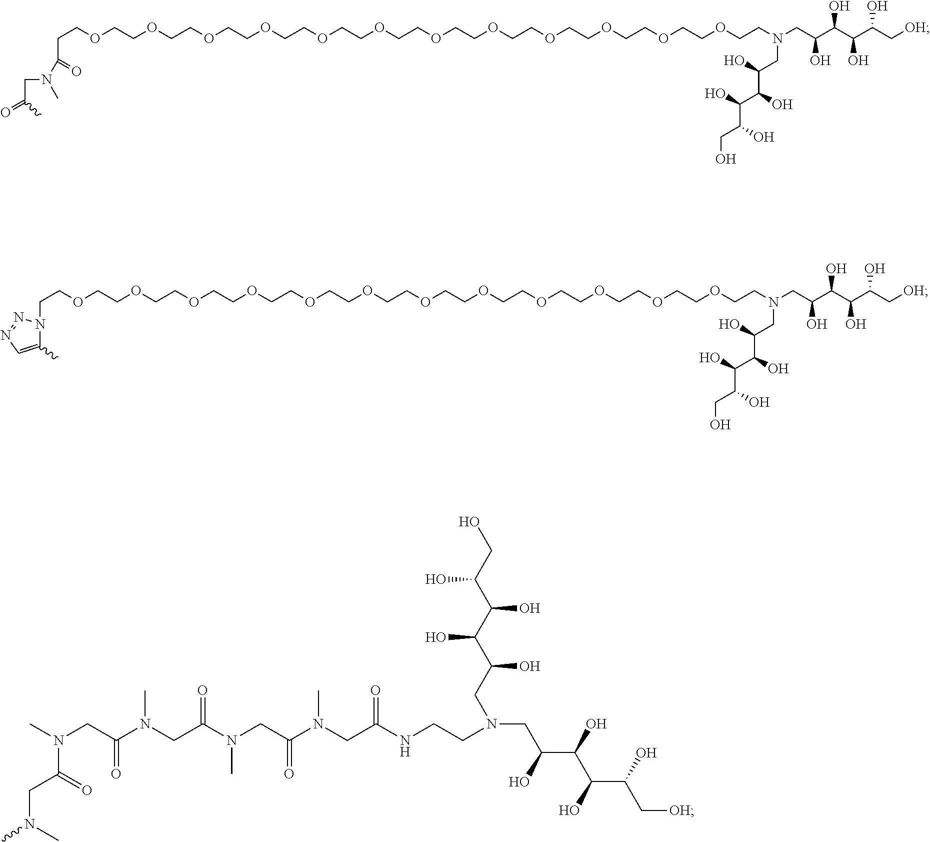

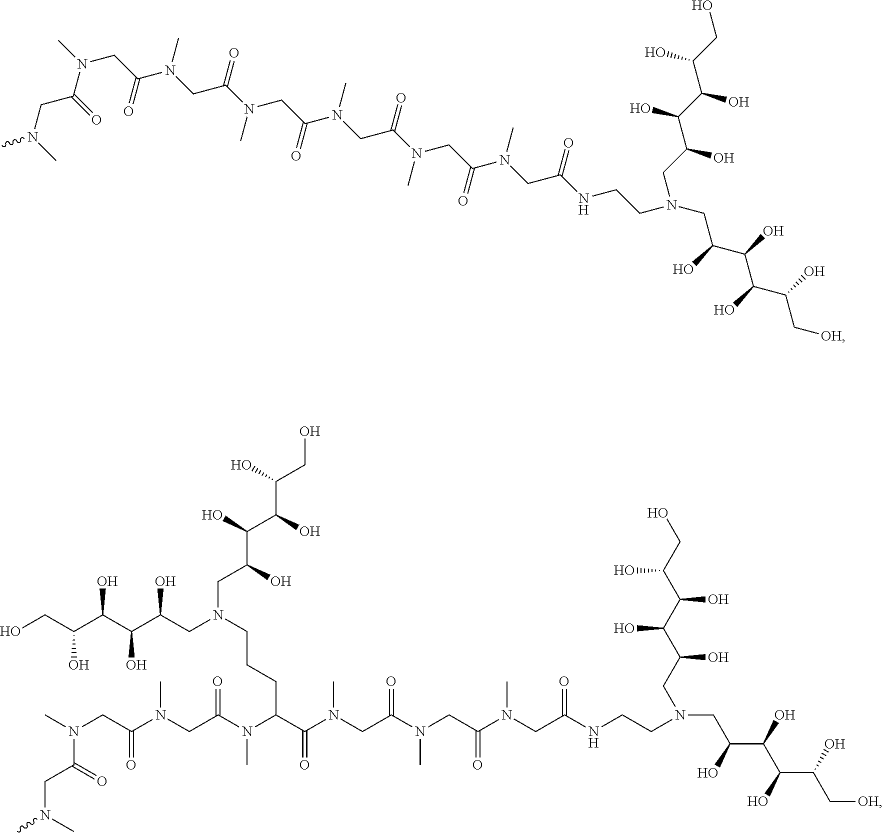

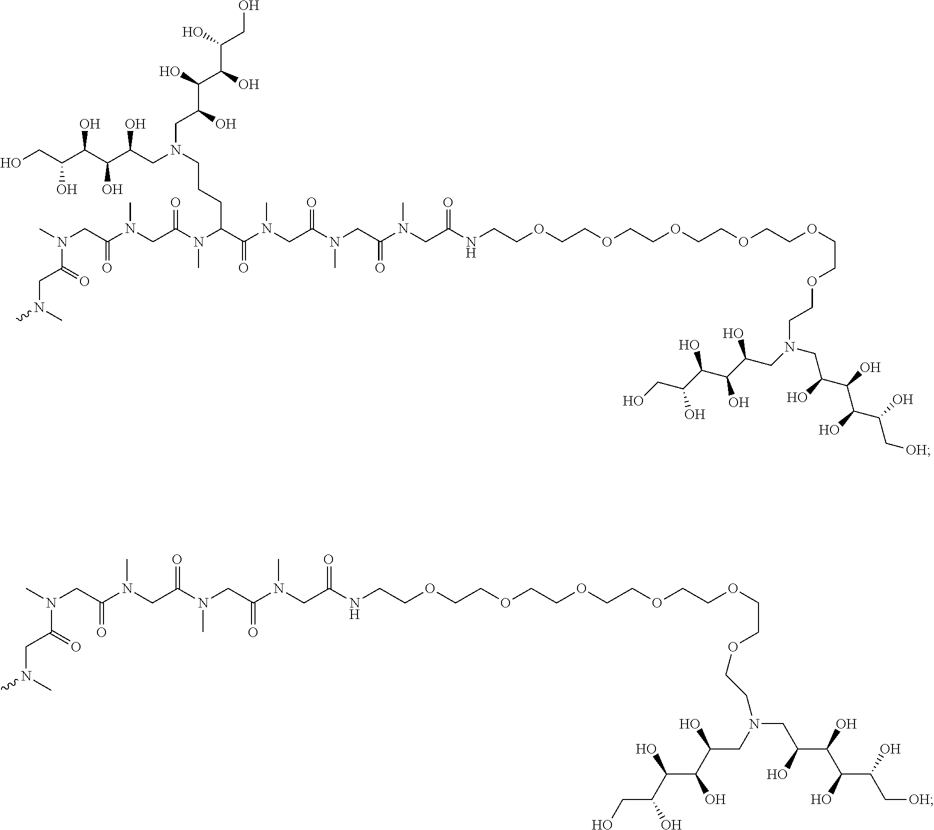

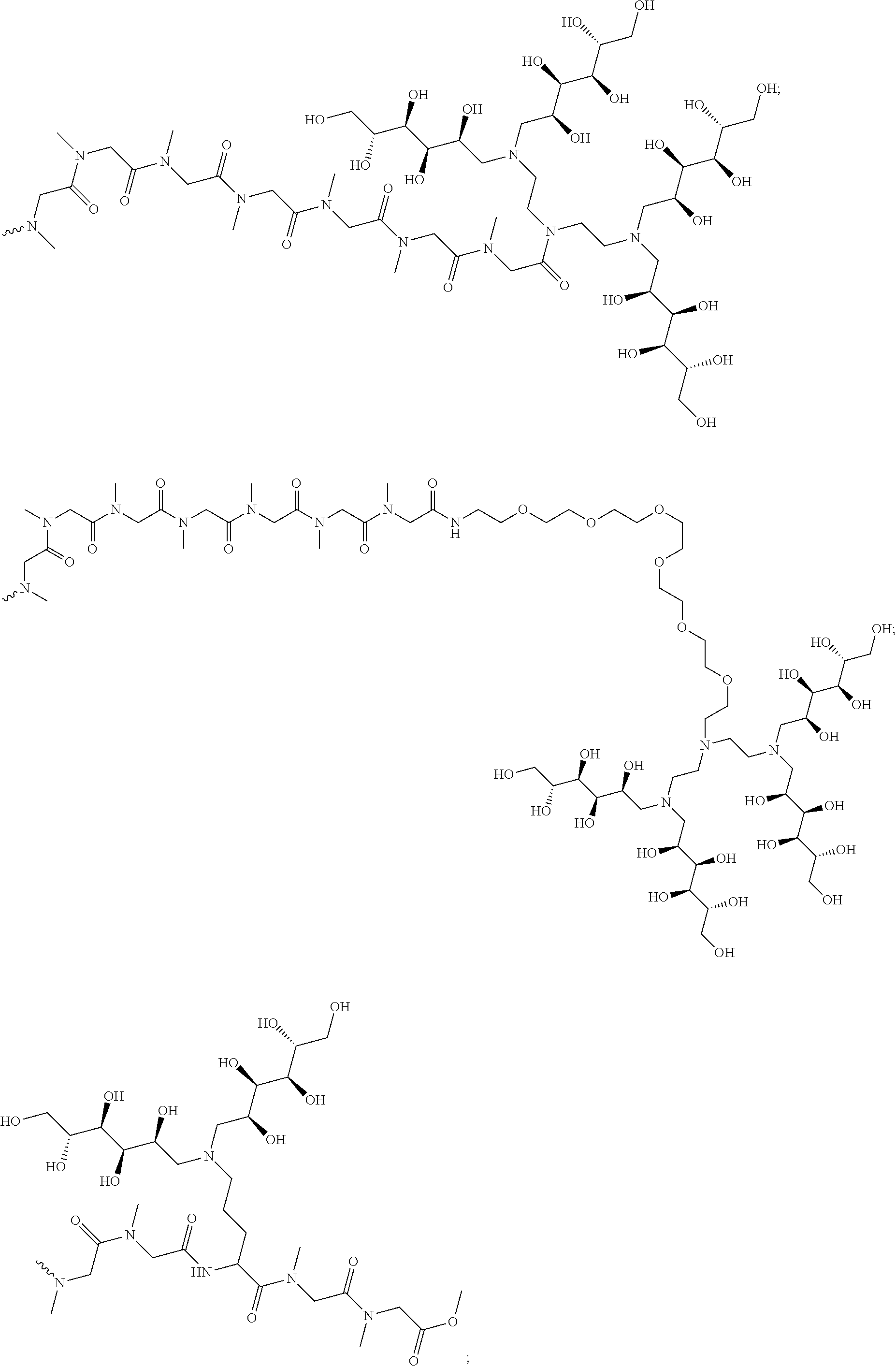

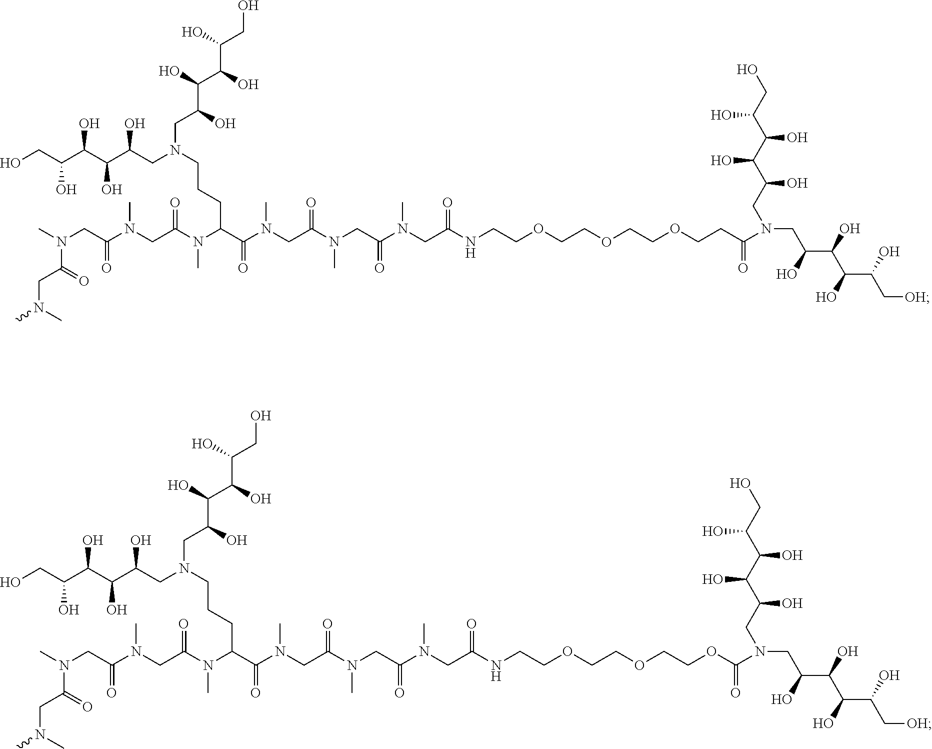

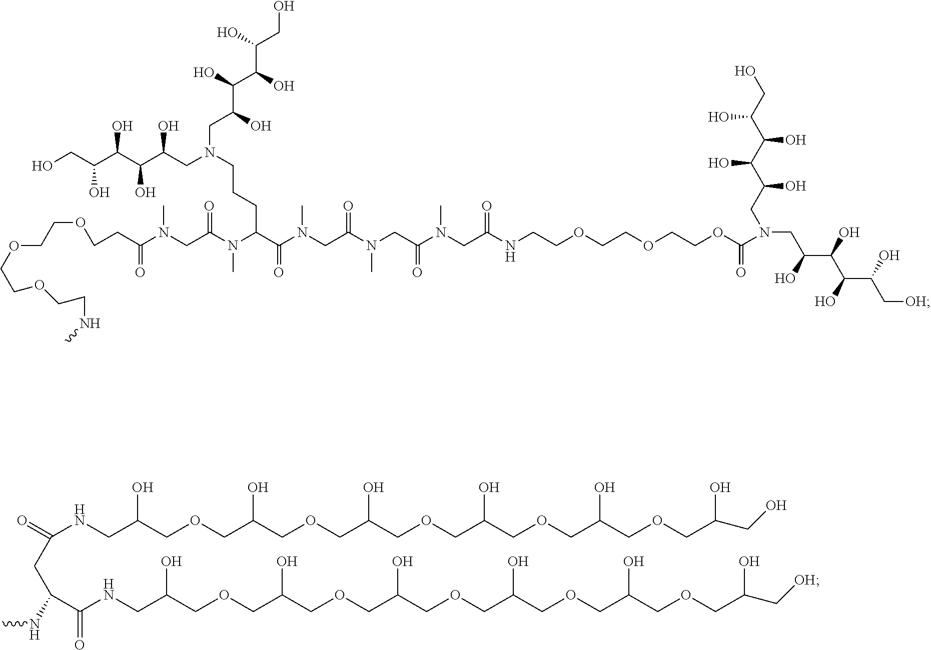

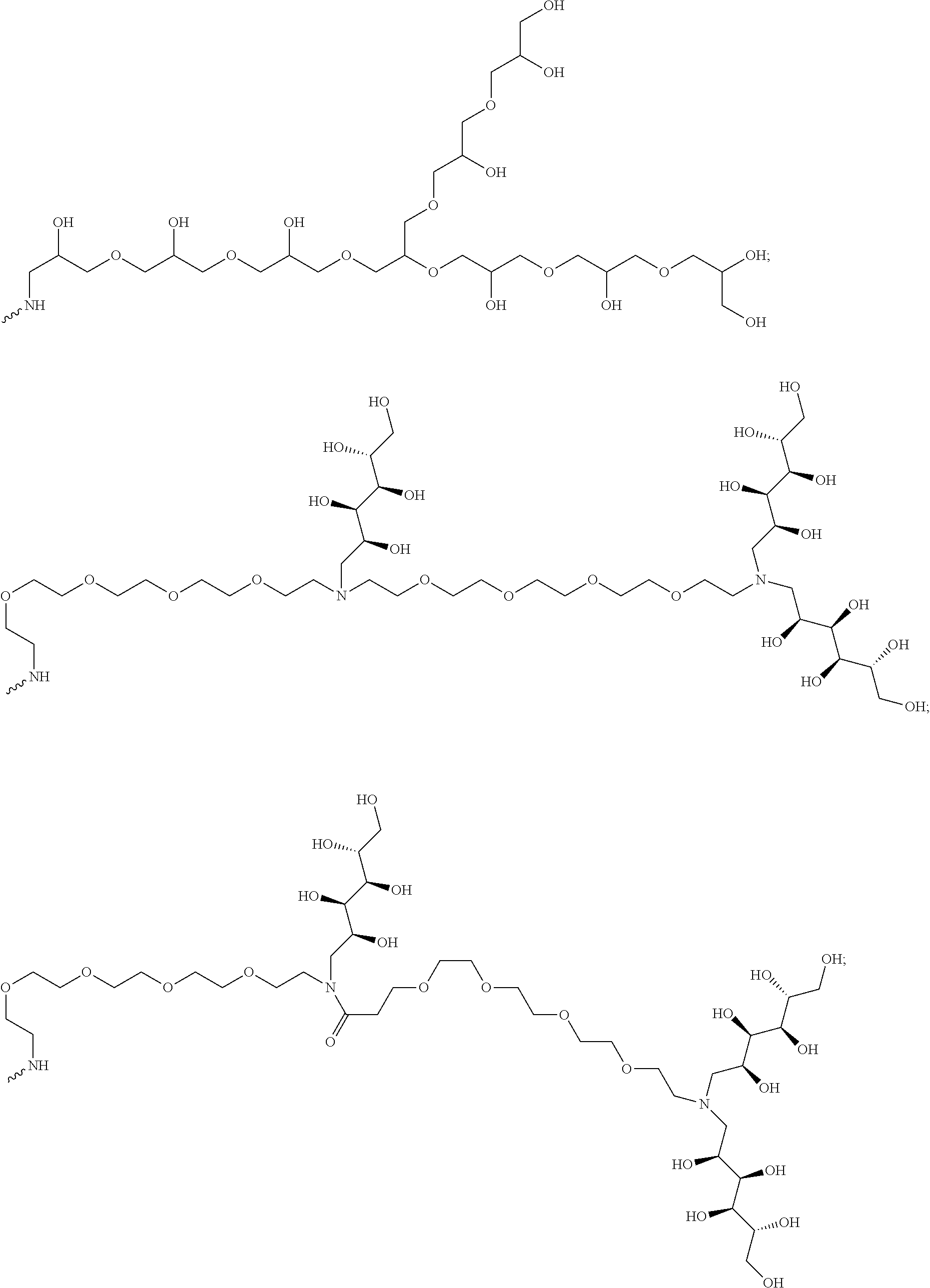

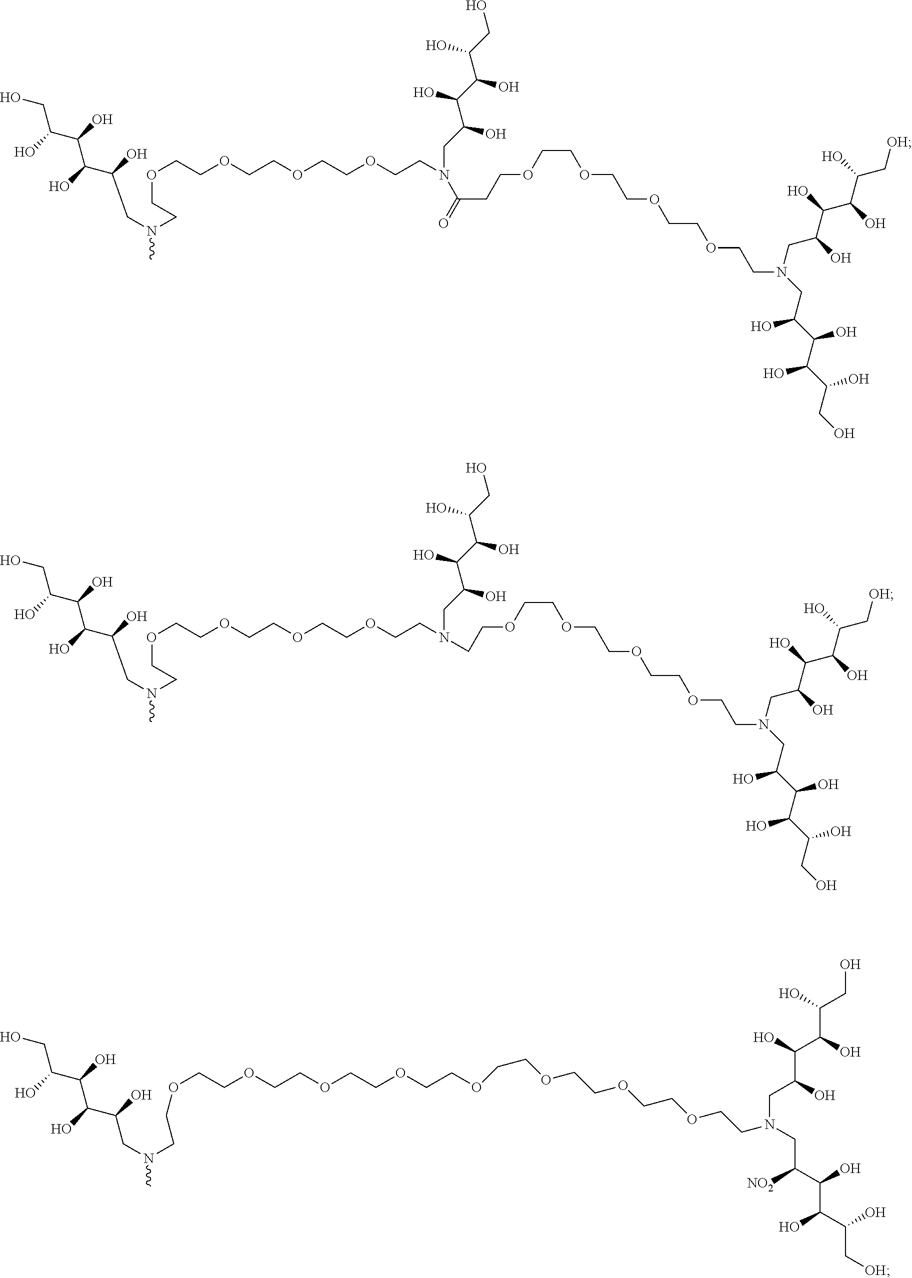

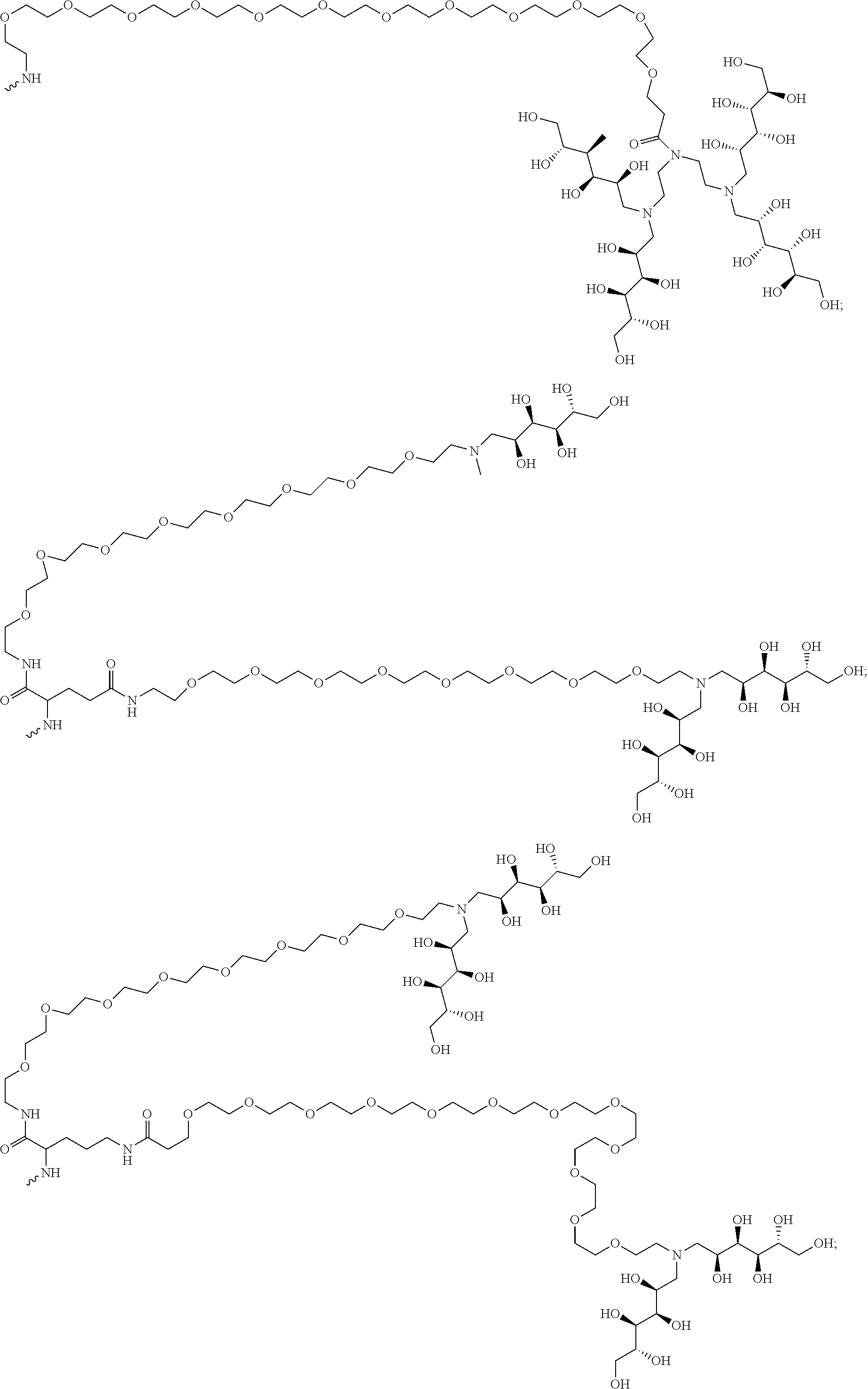

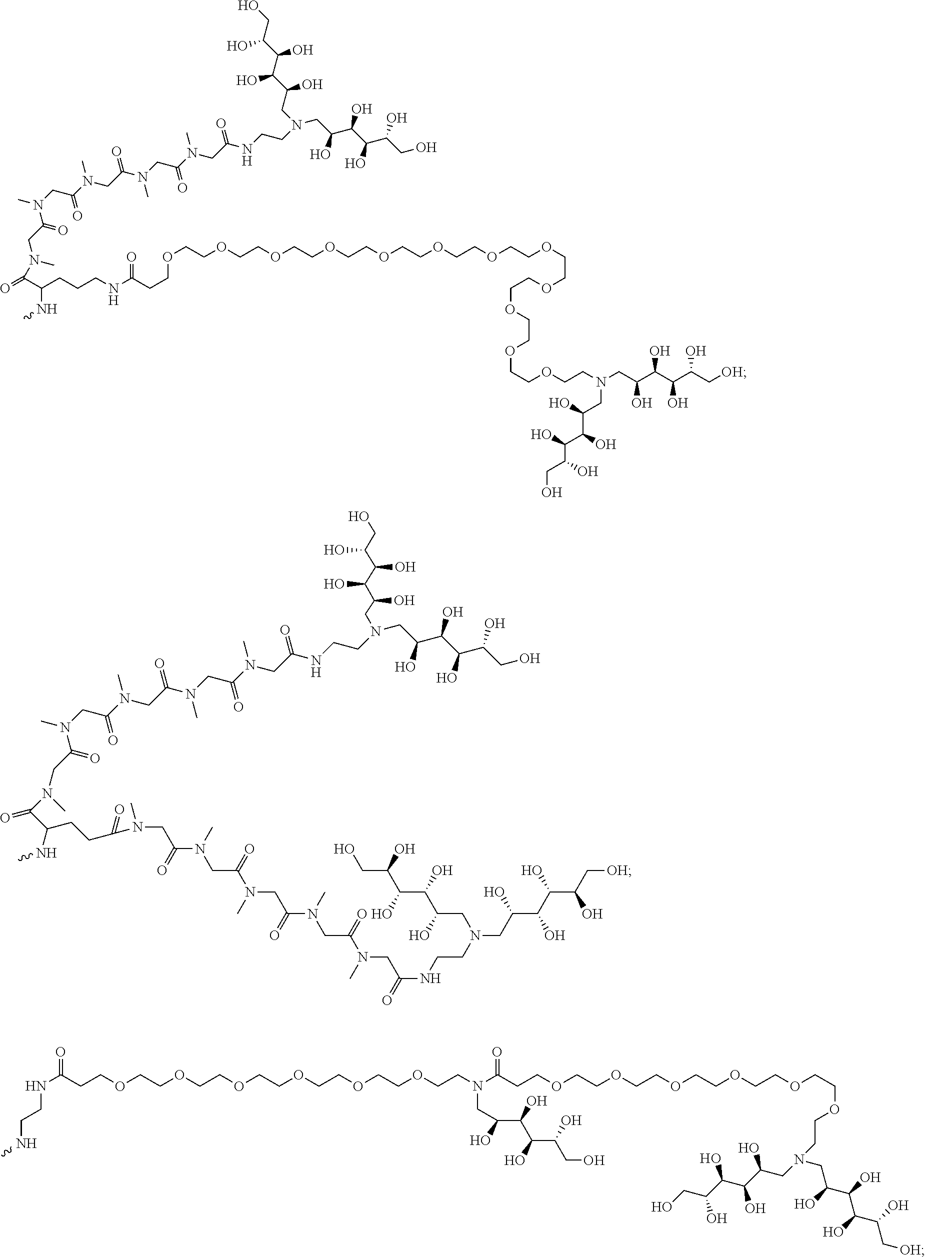

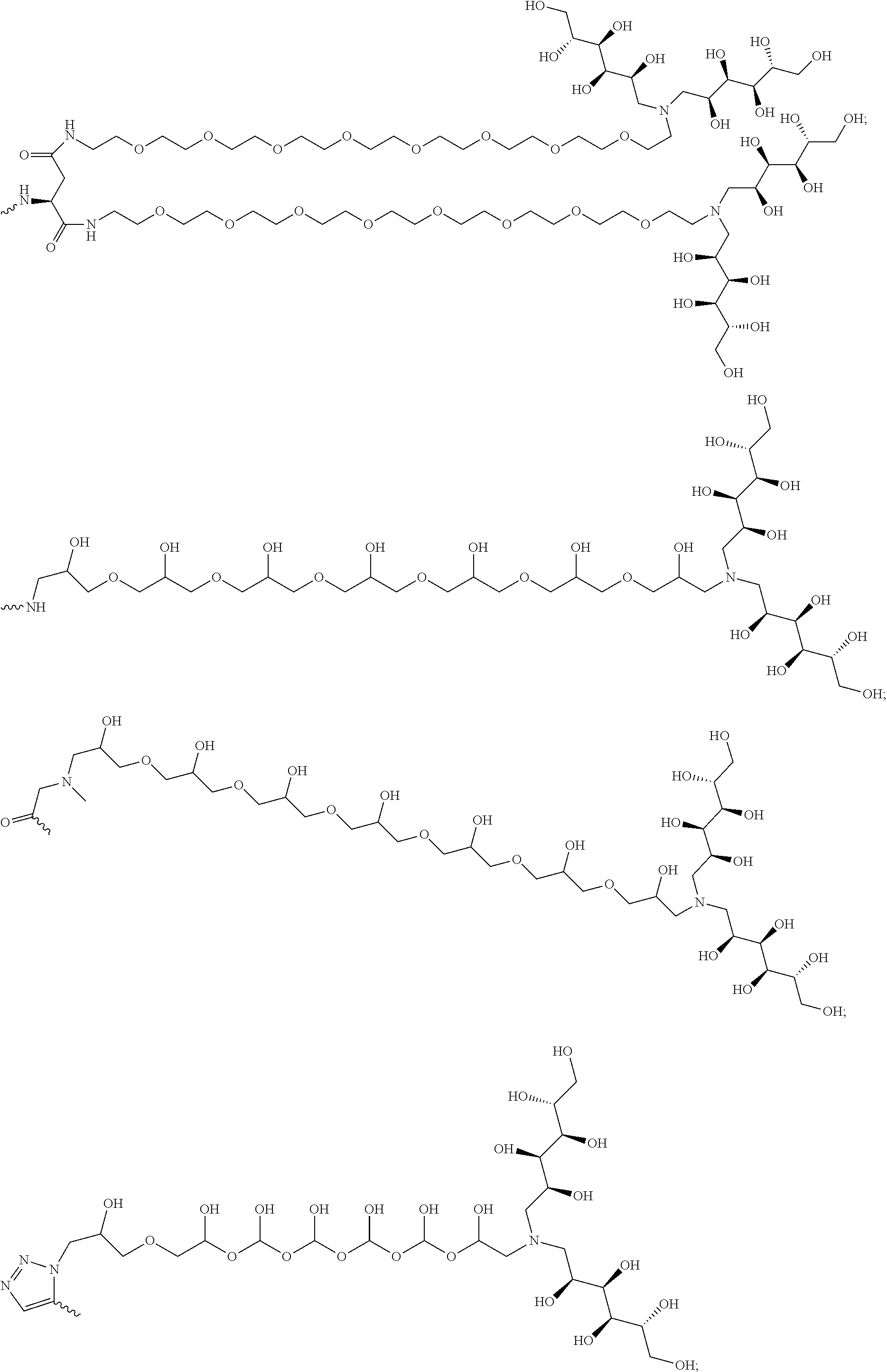

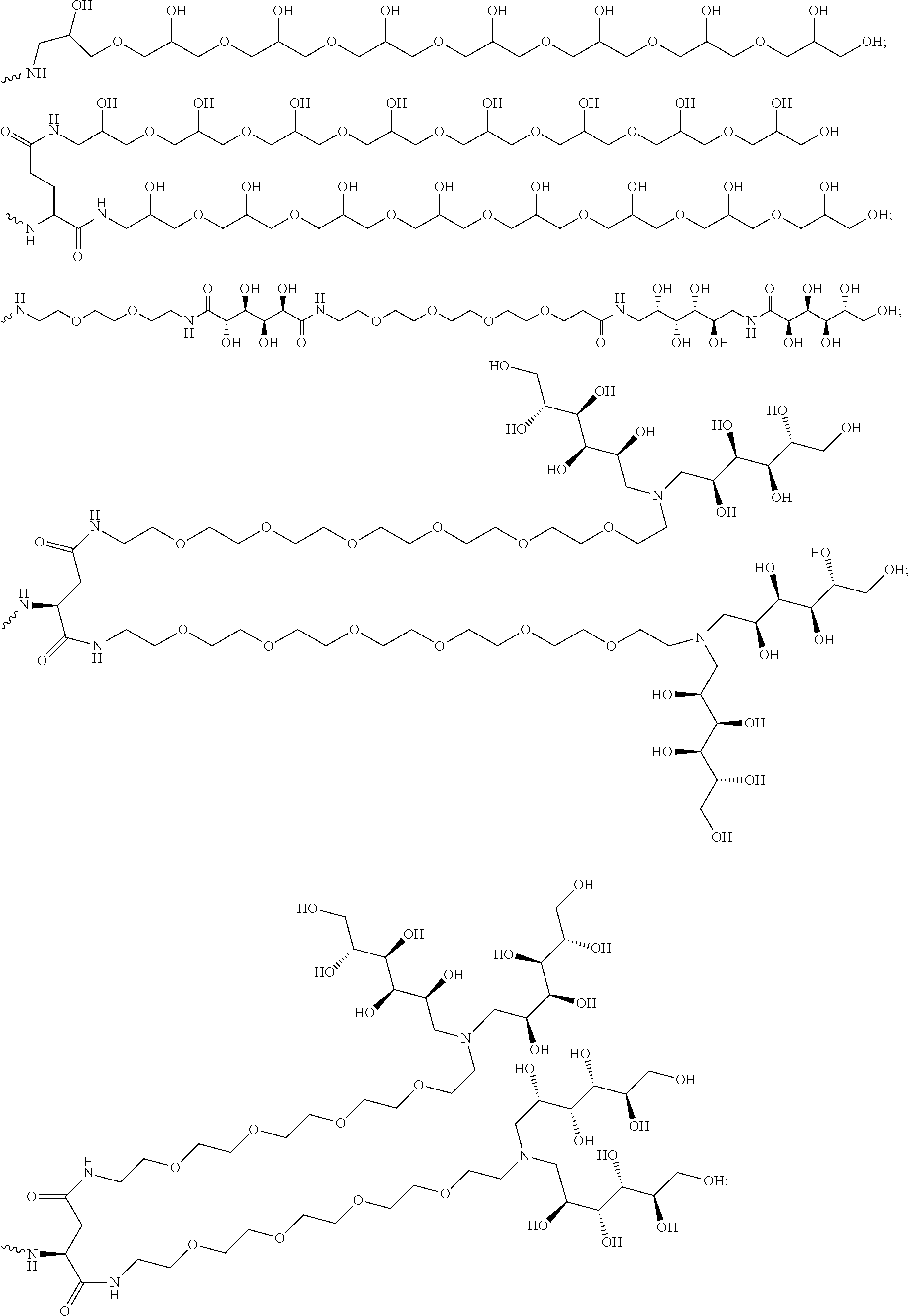

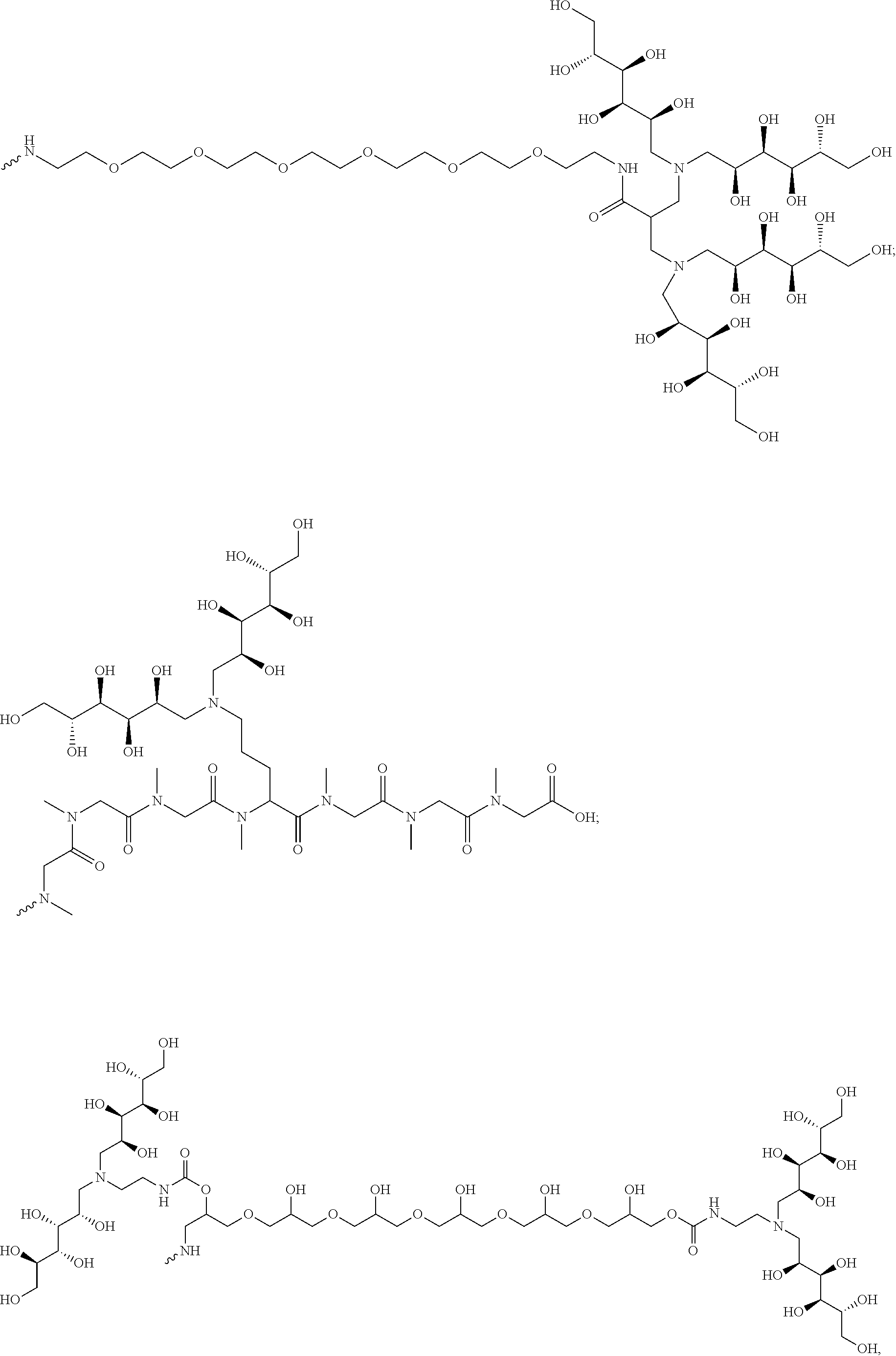

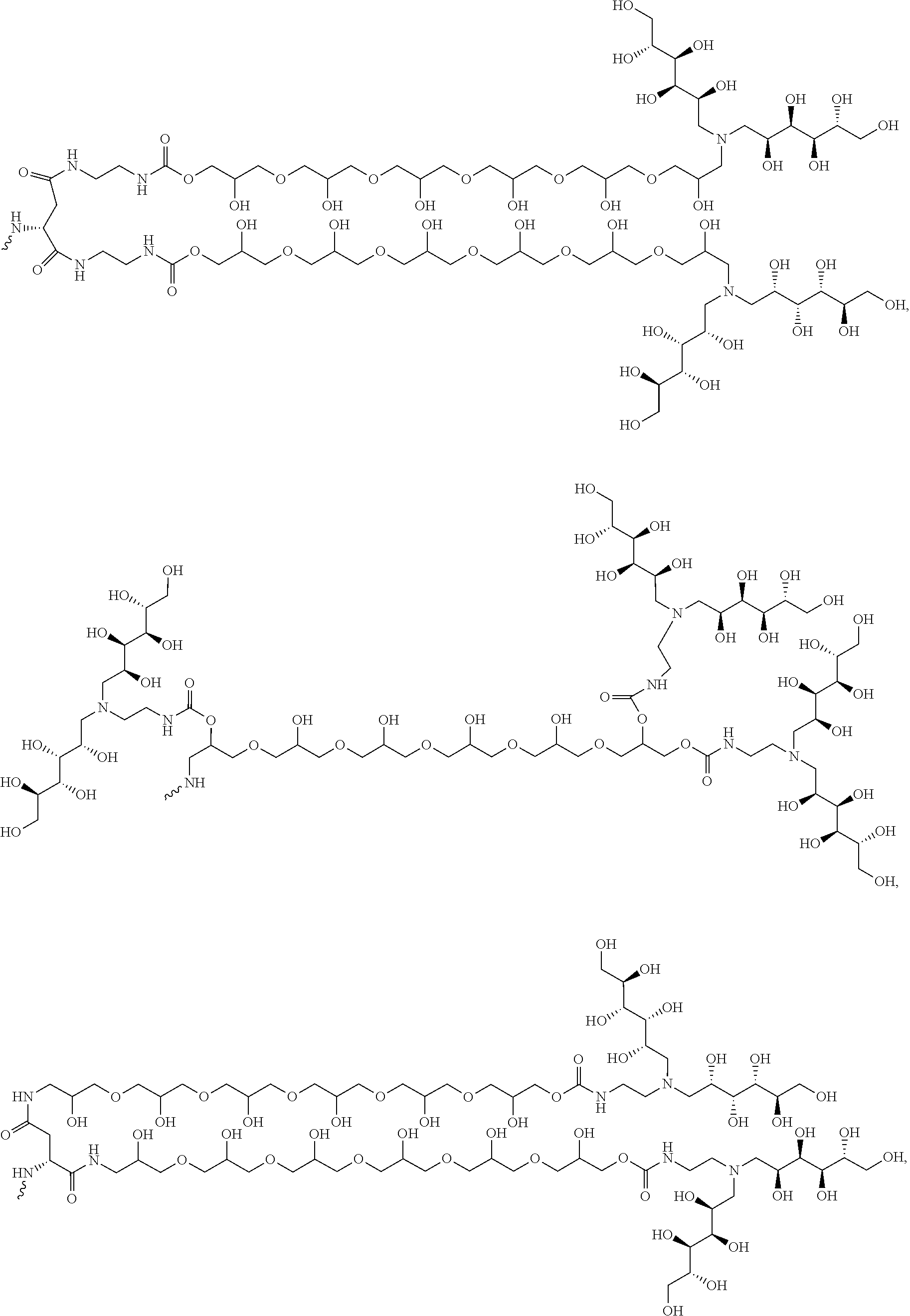

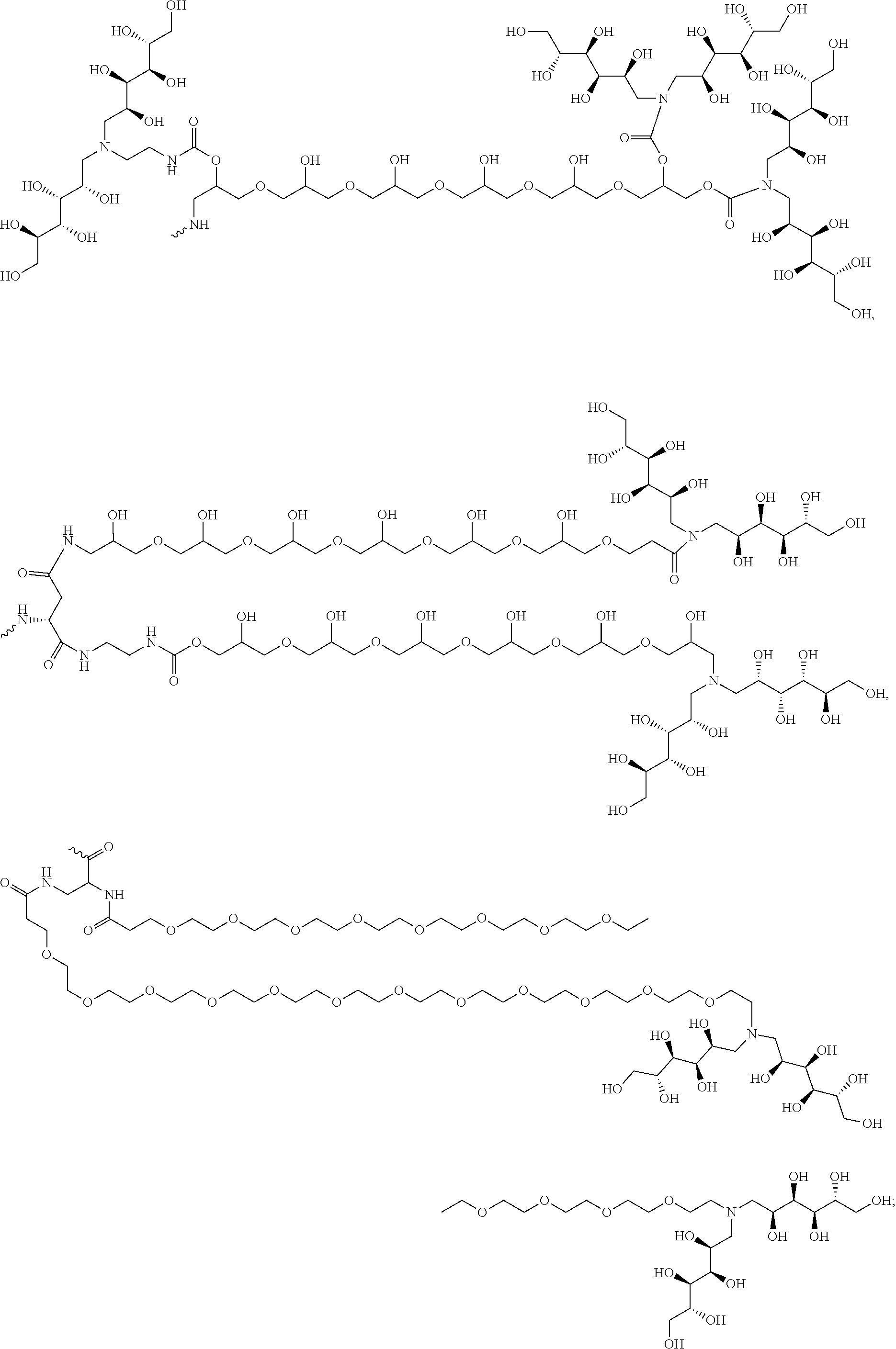

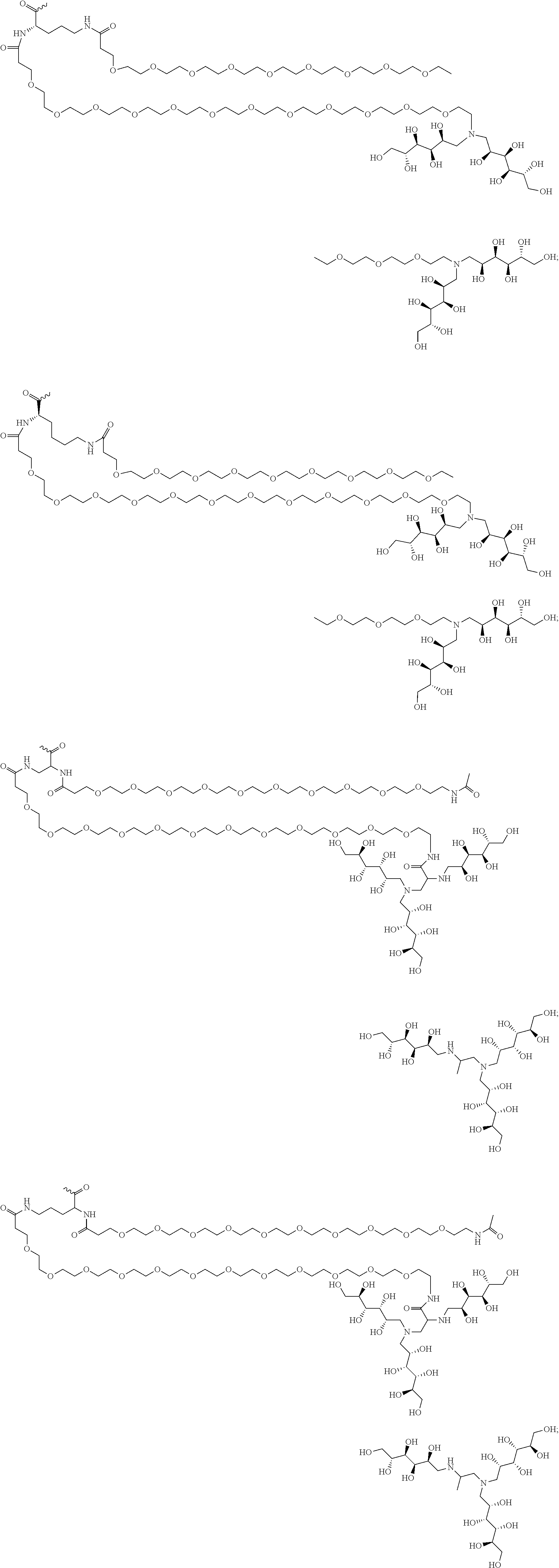

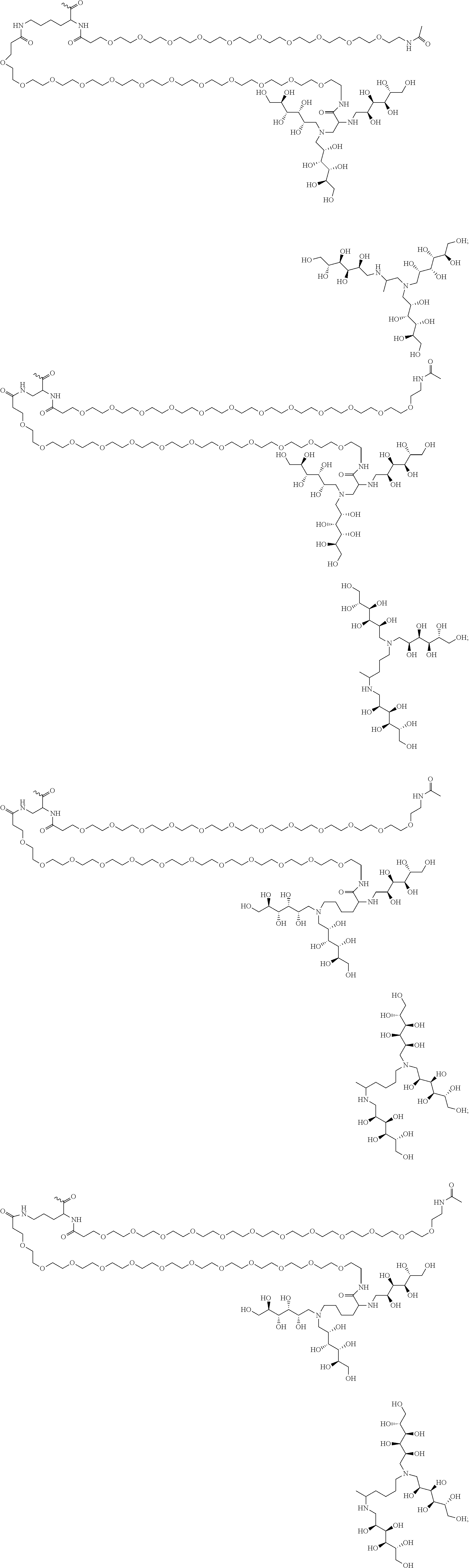

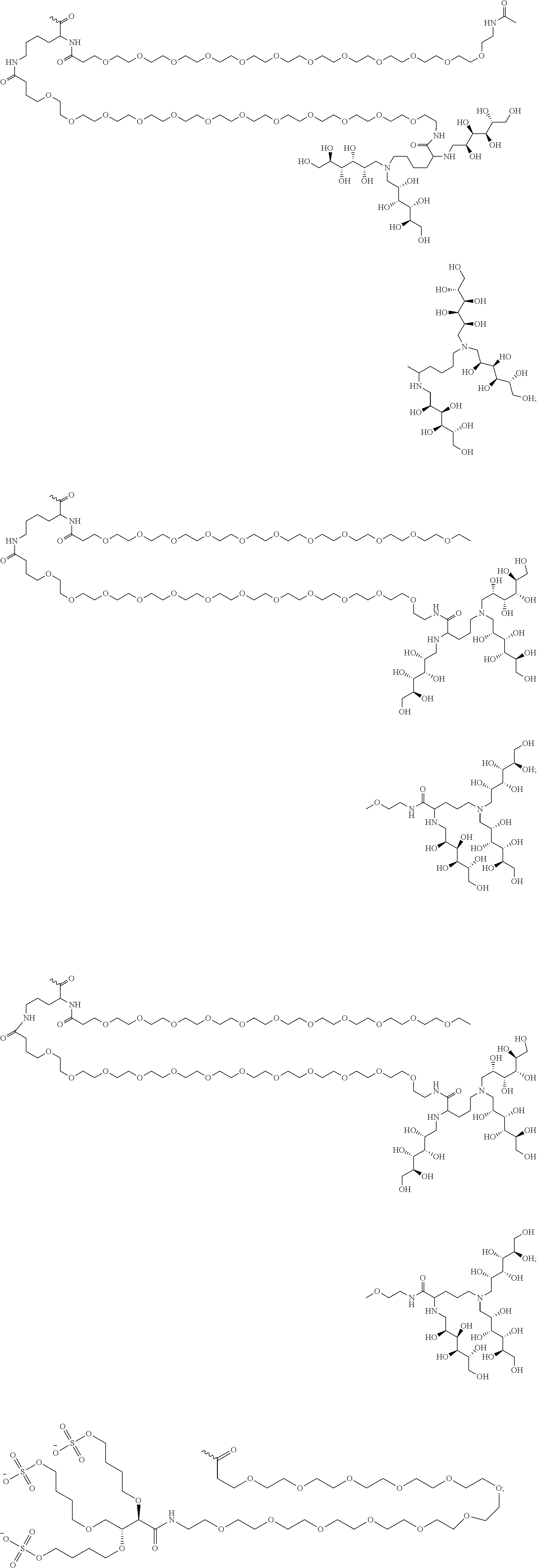

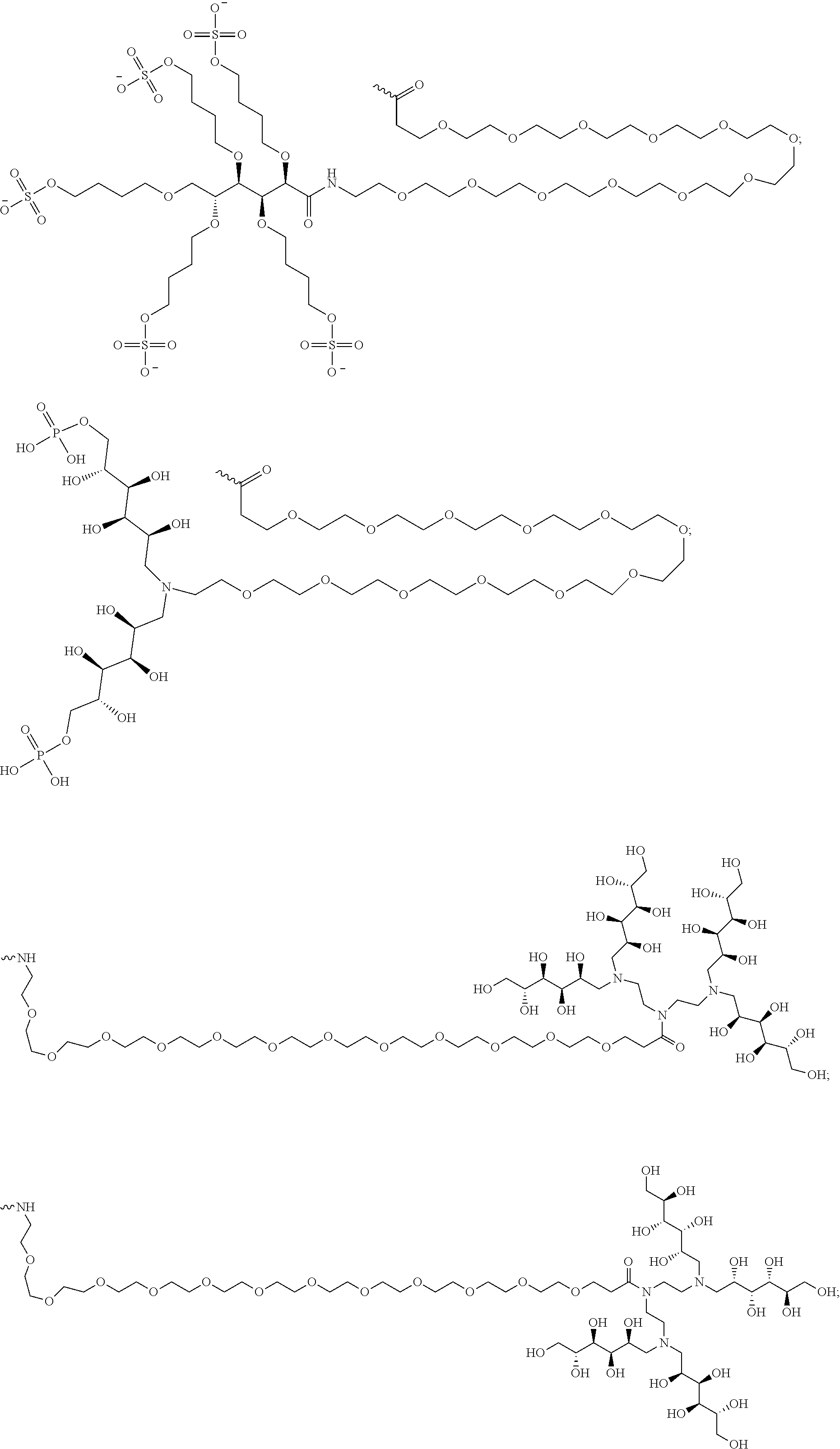

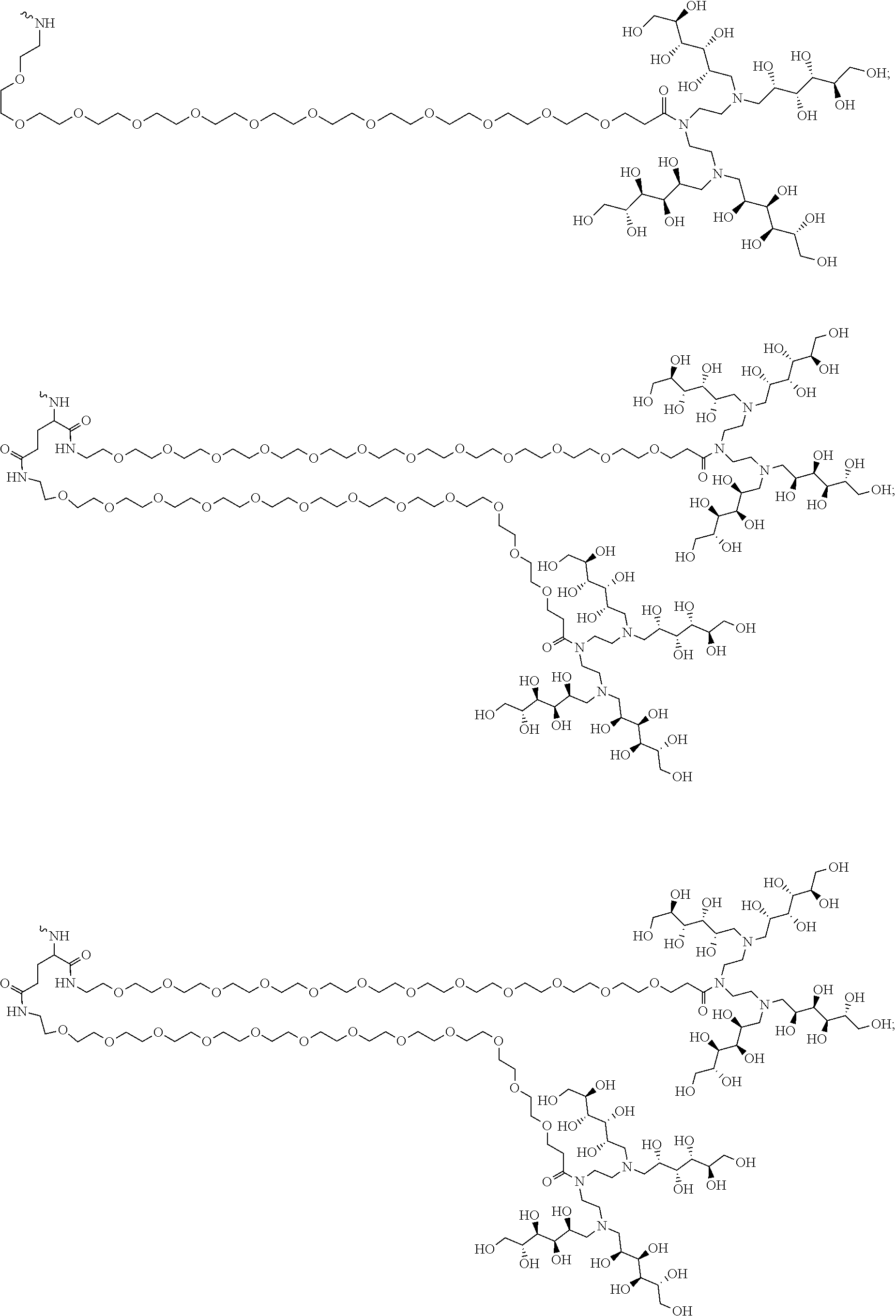

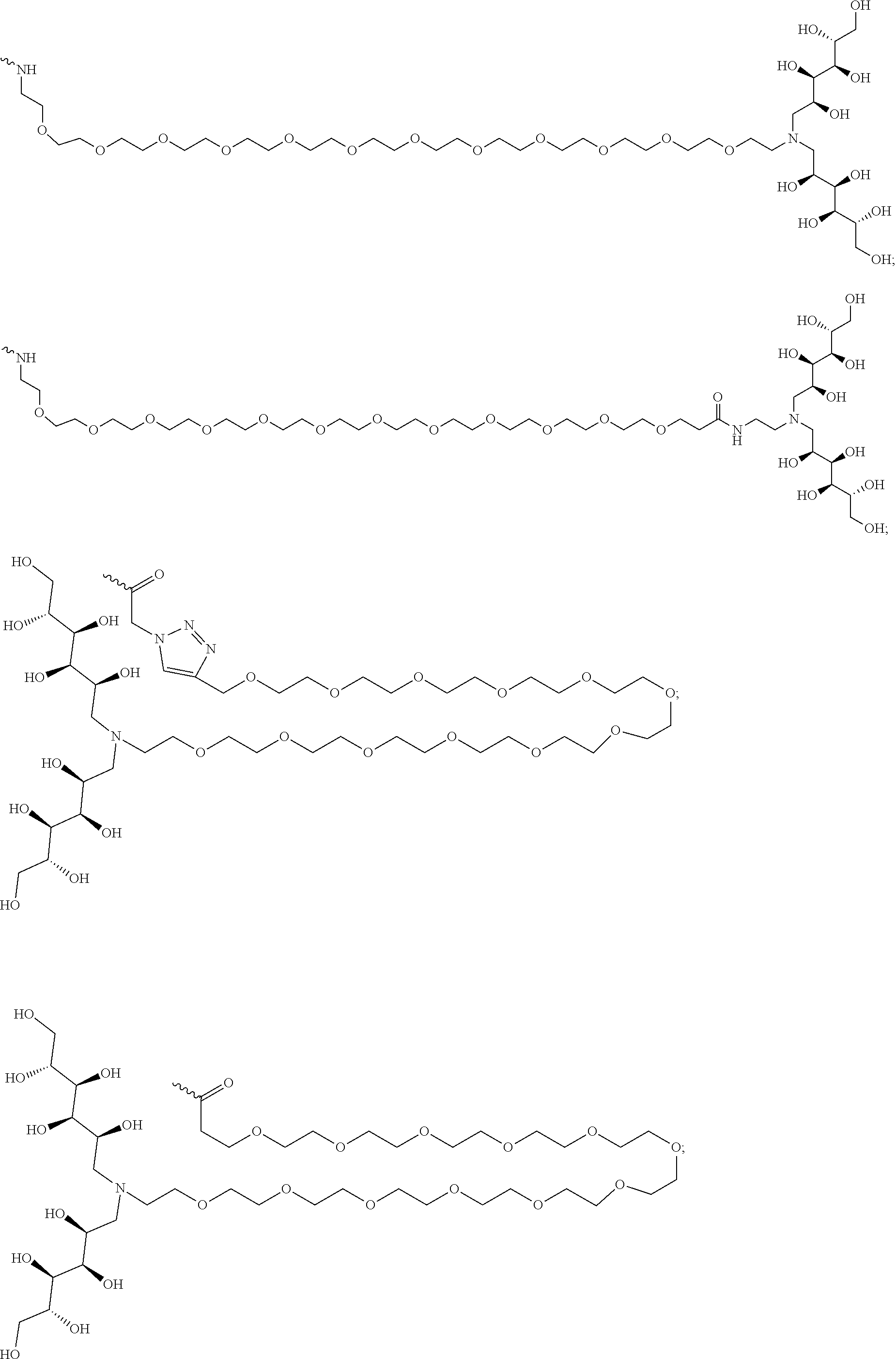

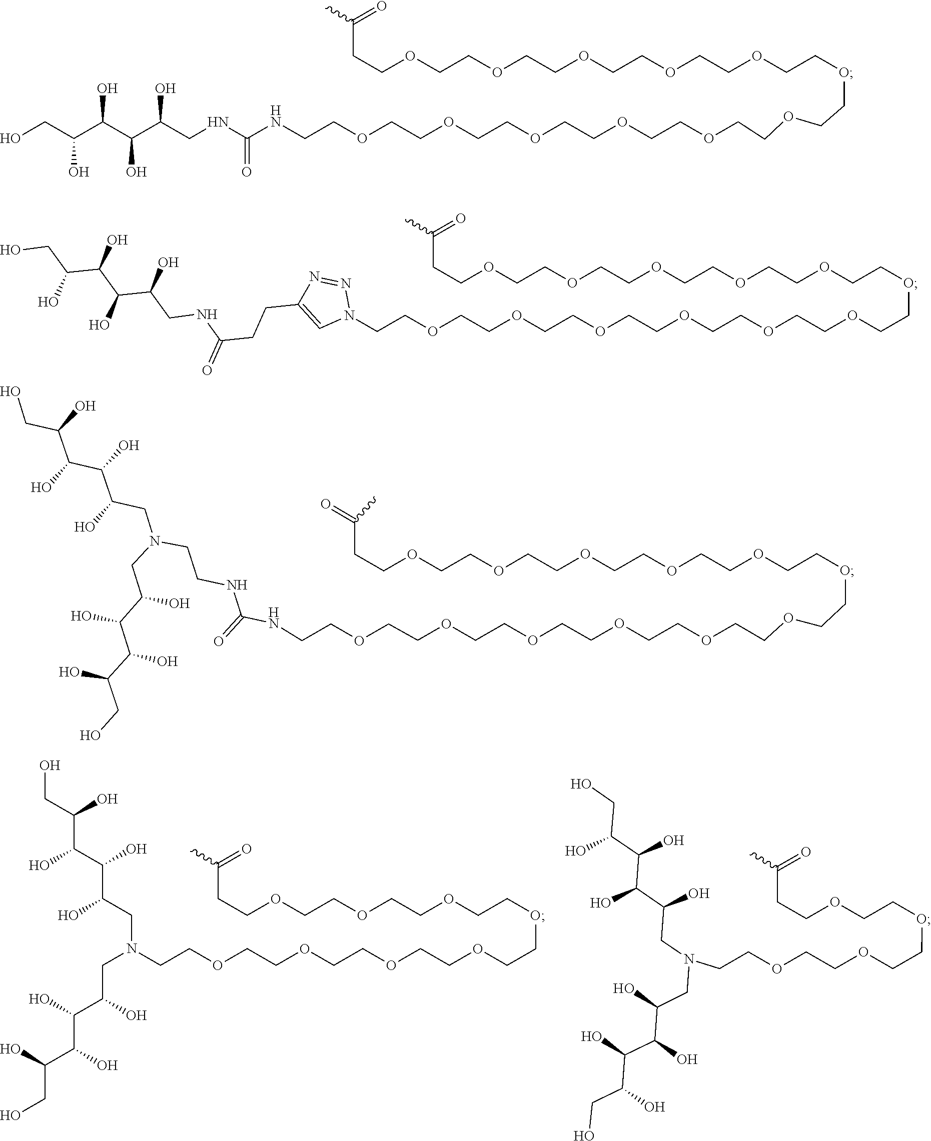

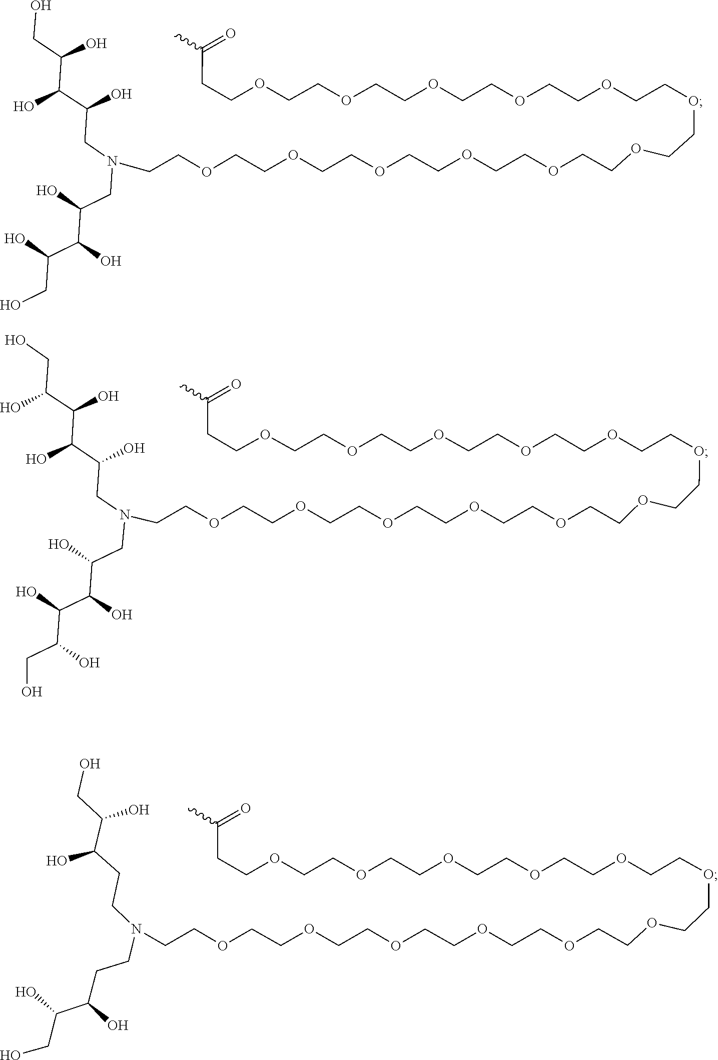

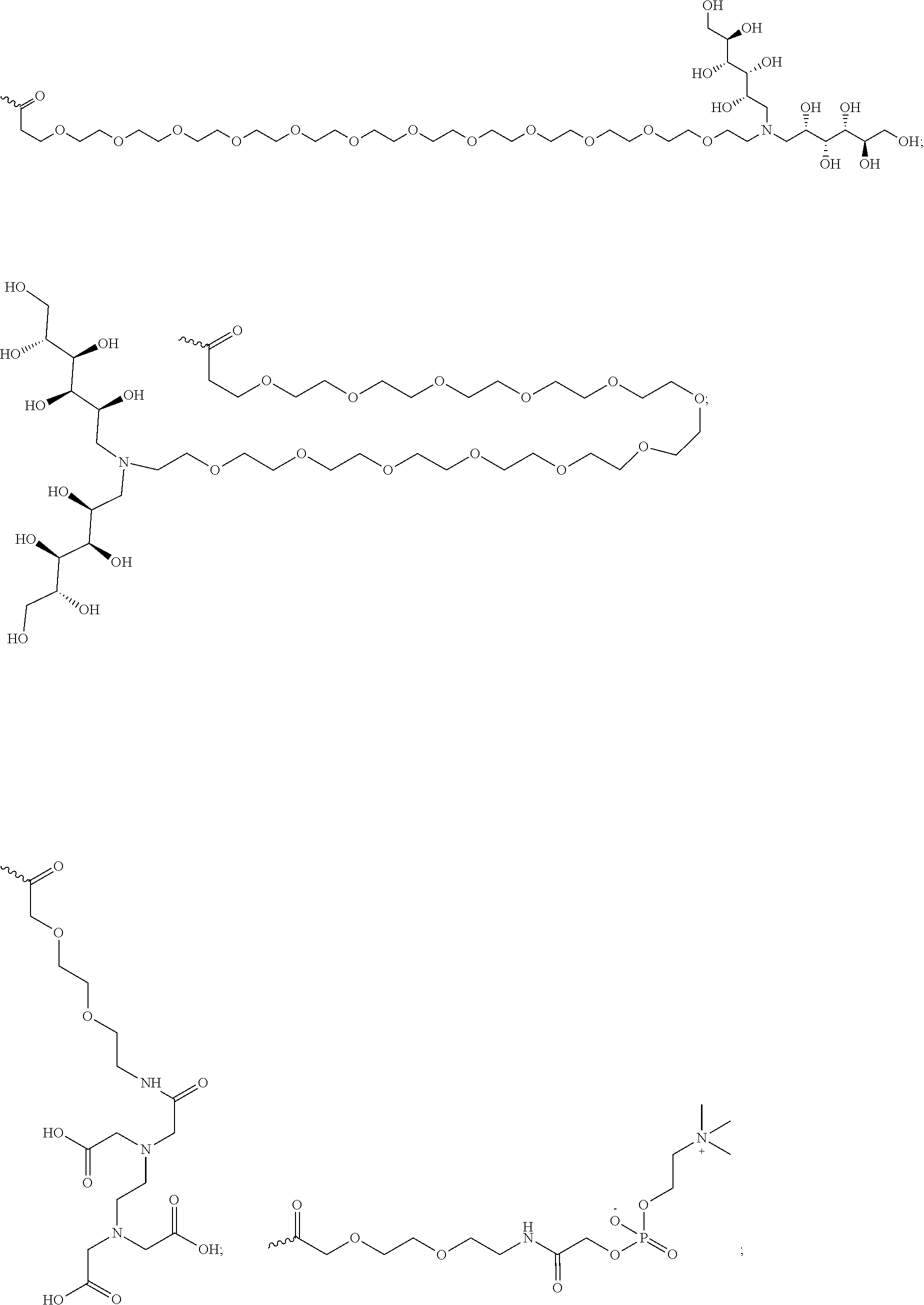

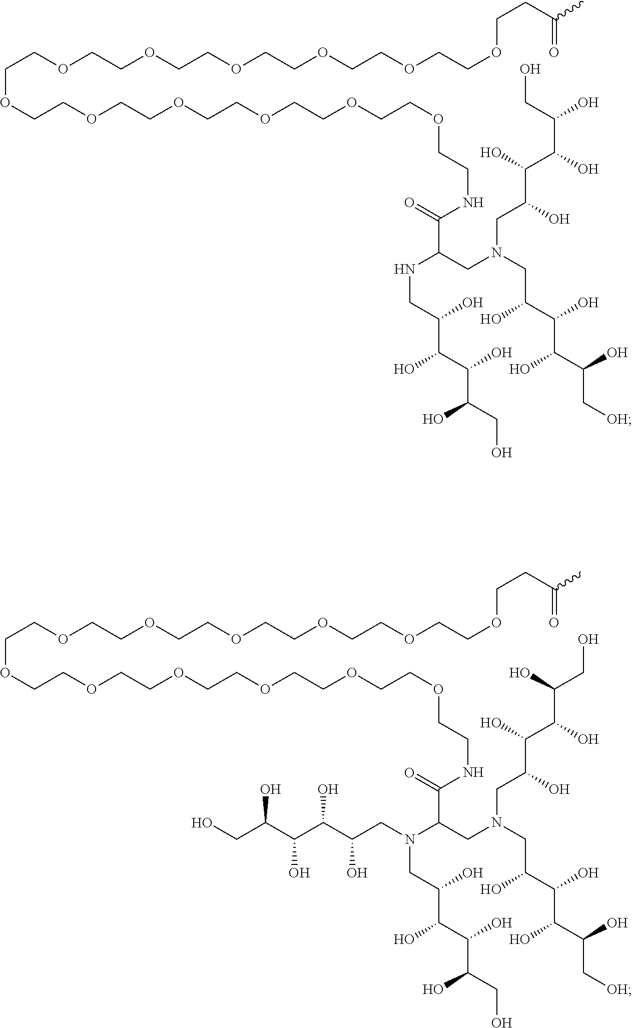

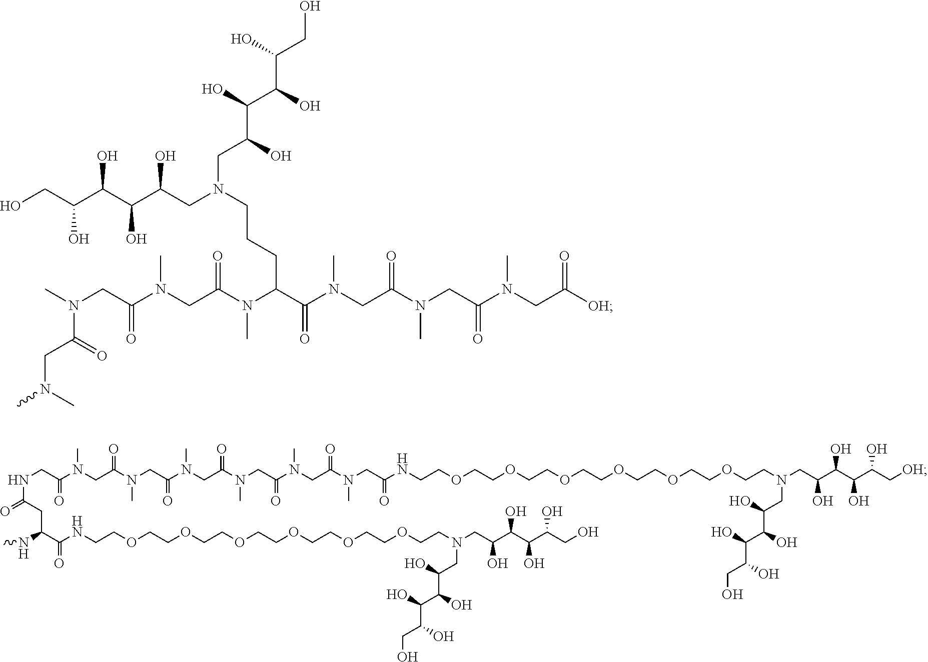

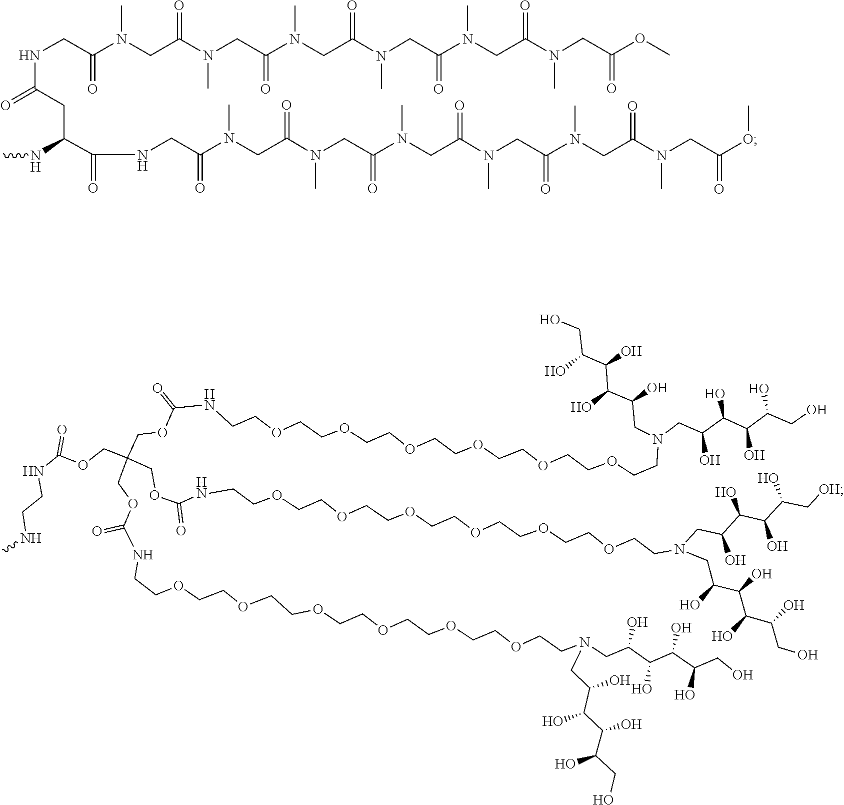

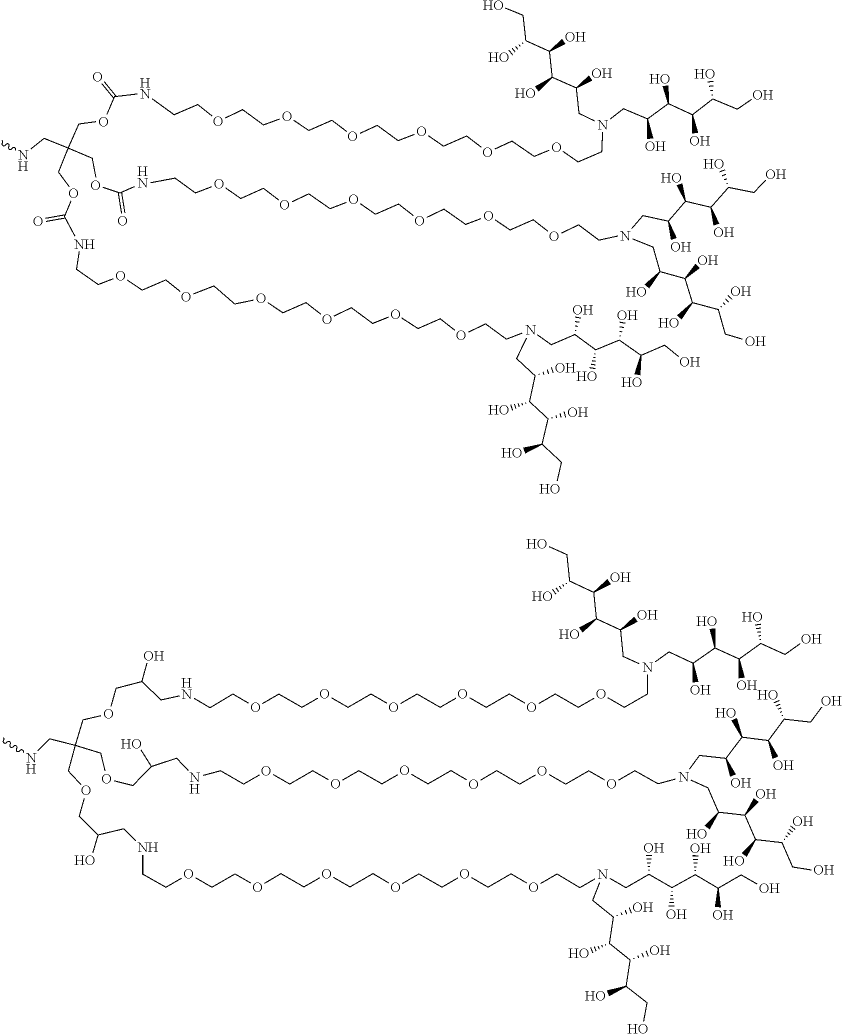

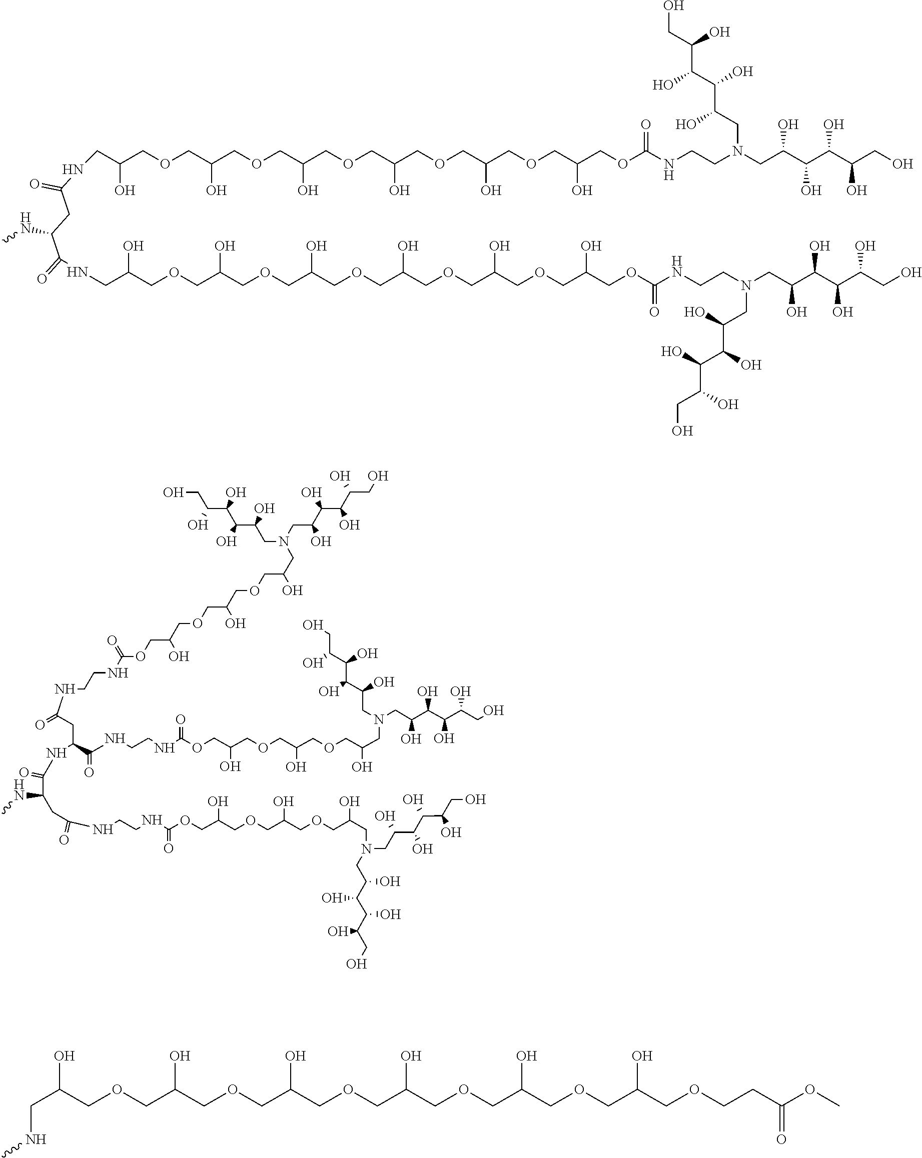

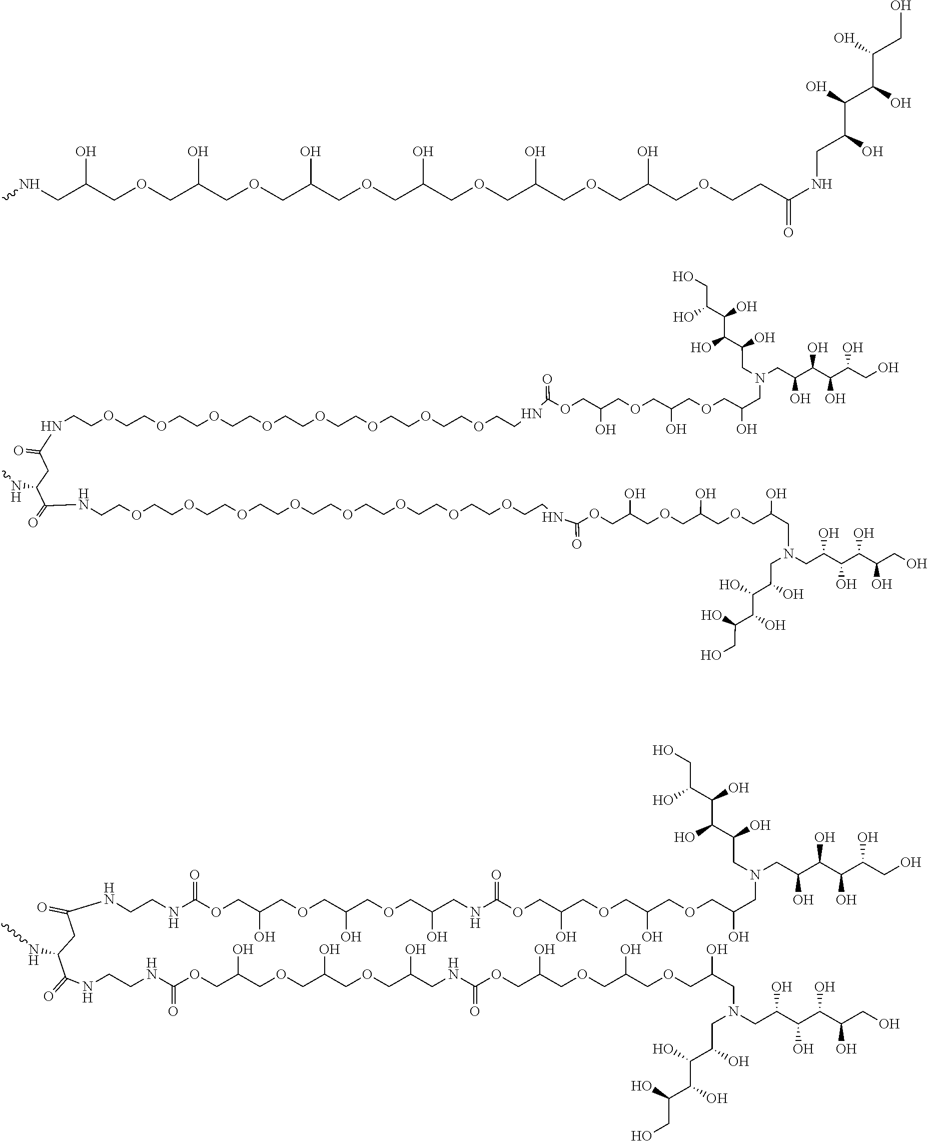

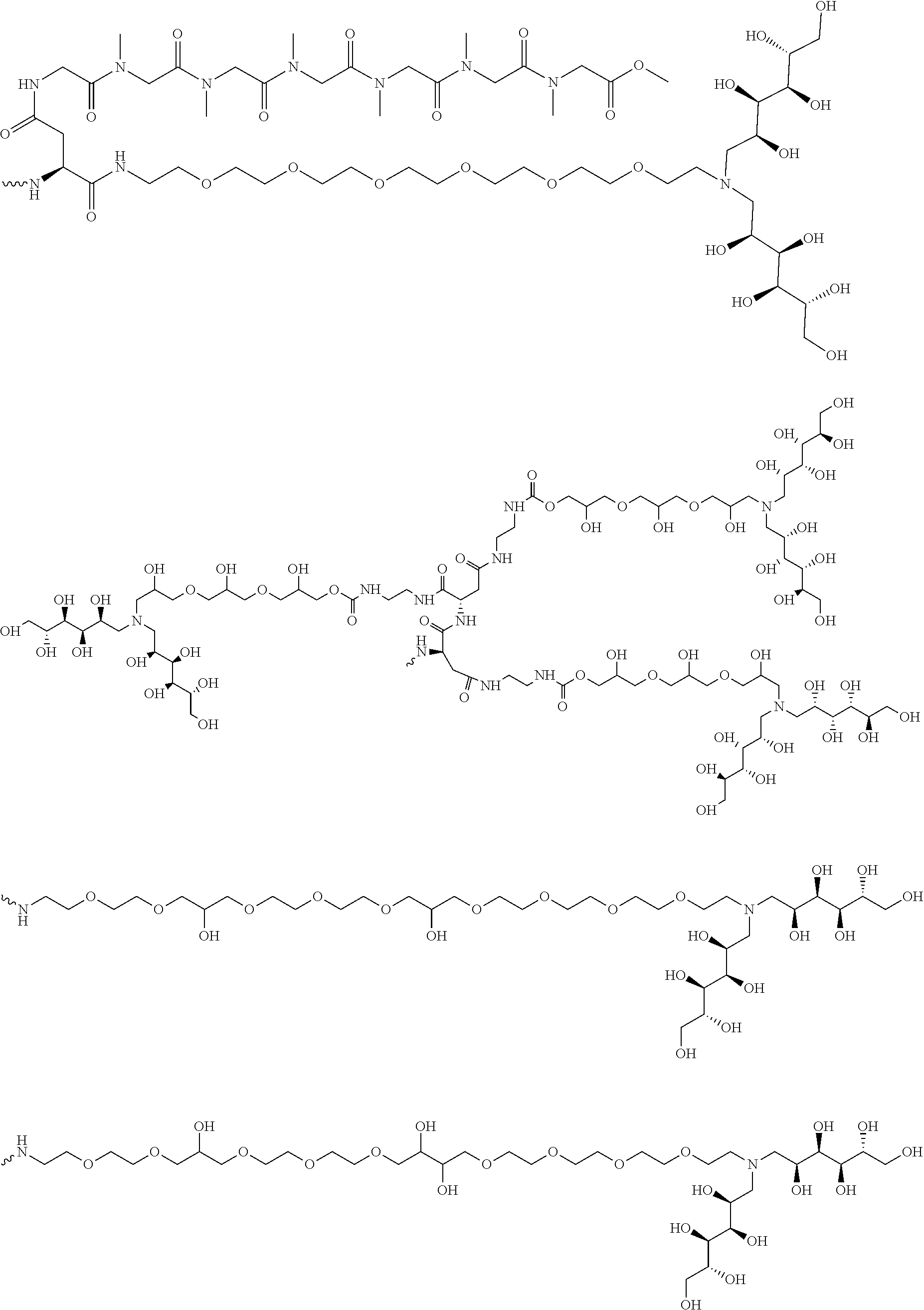

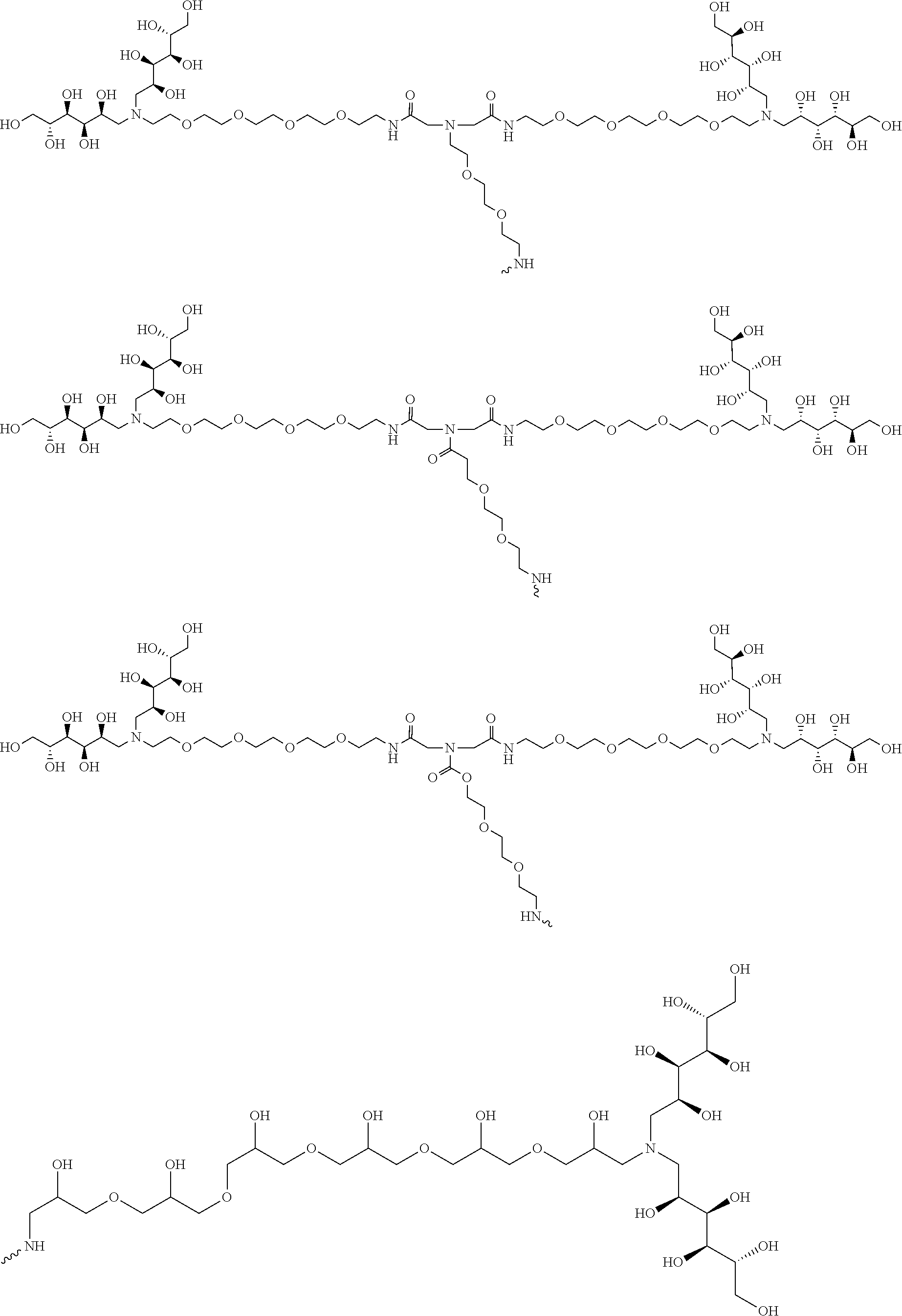

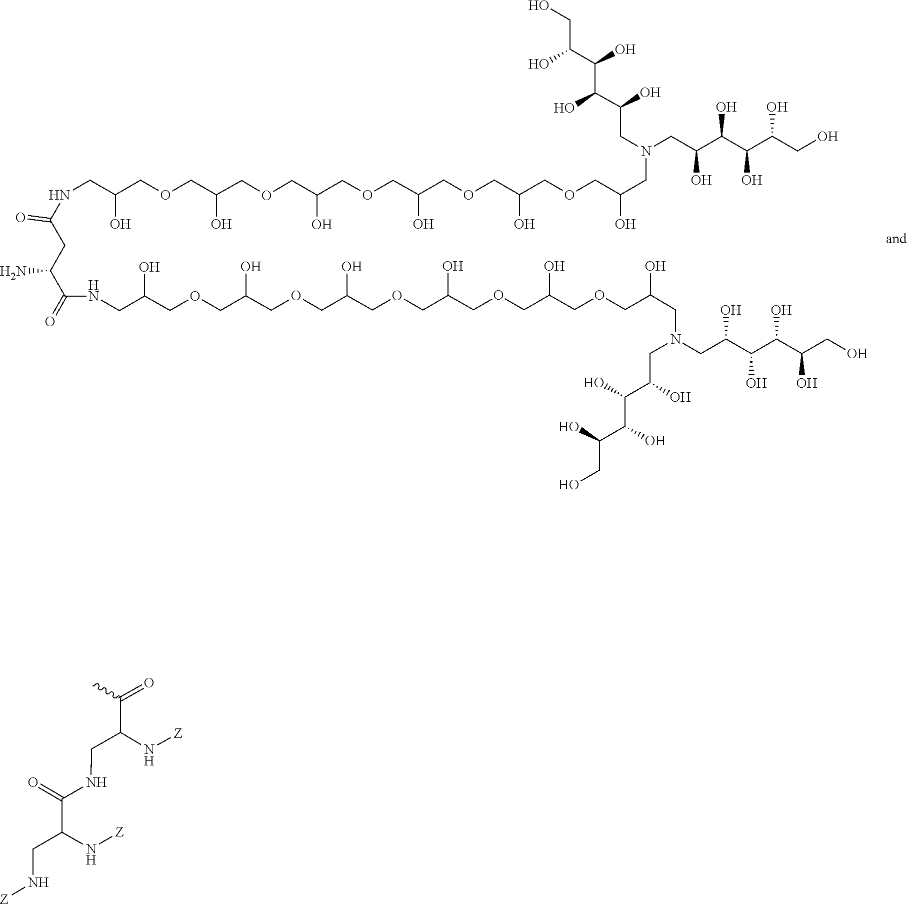

wherein each Z is attached at * and is individually selected from:

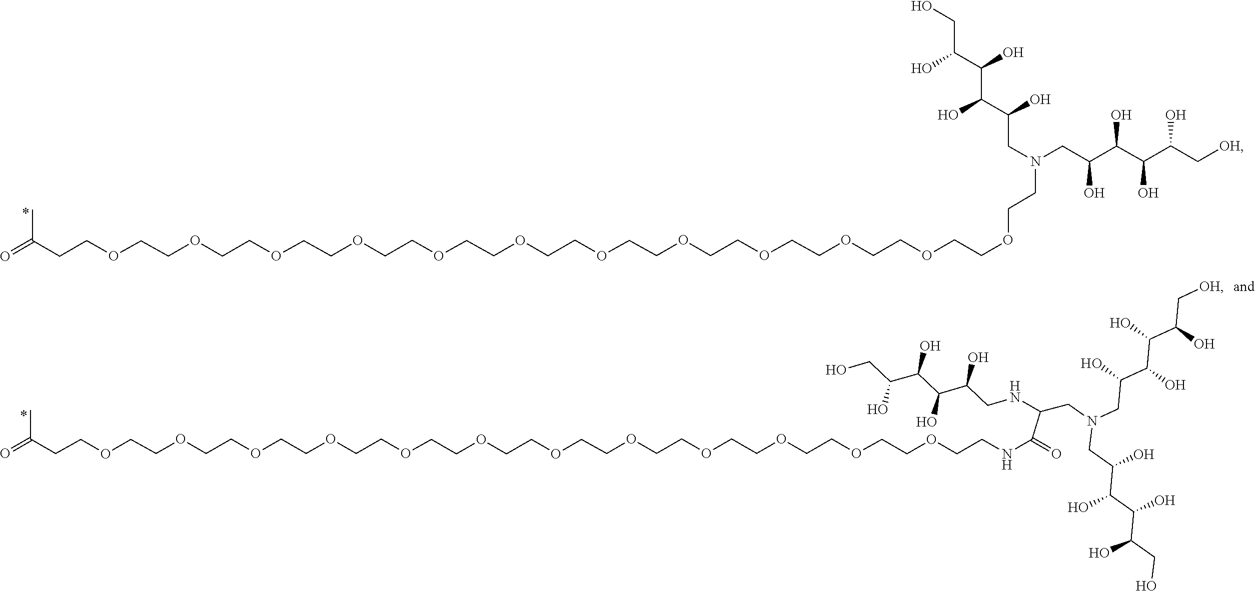 [SMILES: CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; CC(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

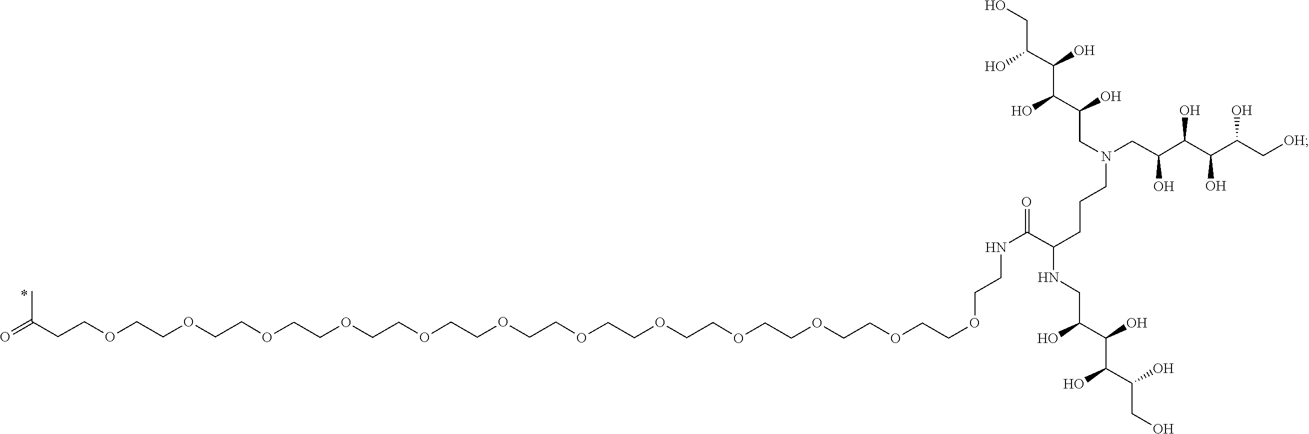 [SMILES: CC(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

wherein each [symbol] is an attachment to site β, to site R^{b}, or to the enzyme-cleavable group.

Embodiment 25. The linker compound of any of Embodiments 1 to 24, comprising a polar group including the polymer unit and a sugar unit.

Embodiment 26. The linker compound of any of Embodiments 1 to 24, comprising a polar group including at least two polymer units.

Embodiment 27. The linker compound of any of Embodiments 1 to 24, comprising a polar group including the polymer unit and a carboxyl unit.

Embodiment 28. The linker compound of any of Embodiments 1 to 24, comprising at least two polar groups.

Embodiment 29. The linker compound of any of Embodiments 1 to 24, comprising a polar group including the polymer unit, the sugar unit and the carboxyl unit.

Embodiment 30. The linker compound of any of Embodiments 1 to 24, comprising a polar group including at least two polymer units, at least one sugar unit and at least one carboxyl unit.

Embodiment 31. The linker compound of any of Embodiments 1 to 24, wherein the enzyme-cleavable group comprises at least two amino acids.

Embodiment 32. The linker compound of any of Embodiments 1 to 31, comprising at least one of the polar groups attached to the enzyme-cleavable group.

Embodiment 33. The linker compound of any of Embodiments 1-32, having one of the following structures:

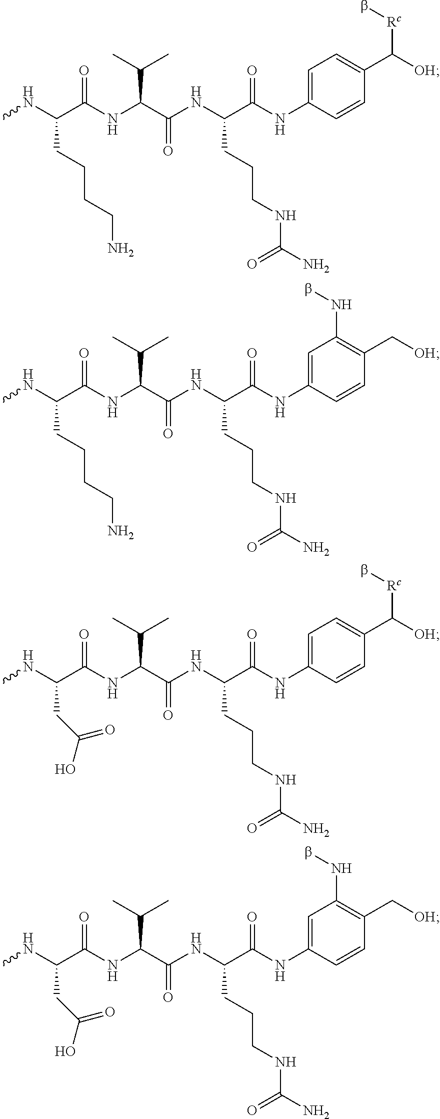

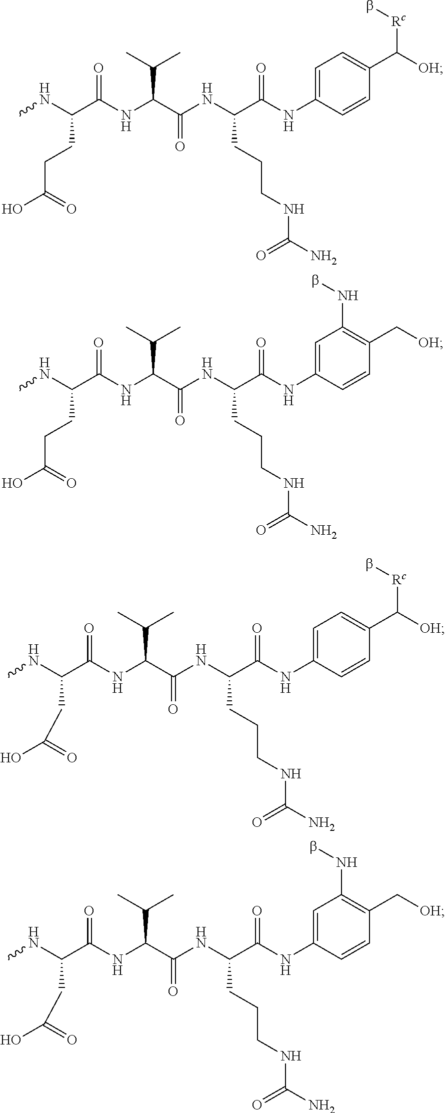

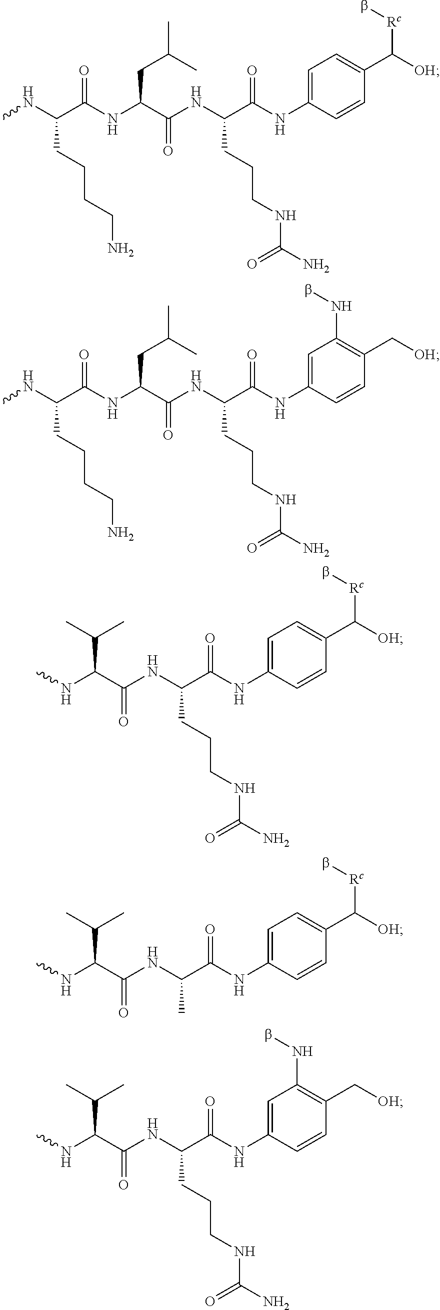

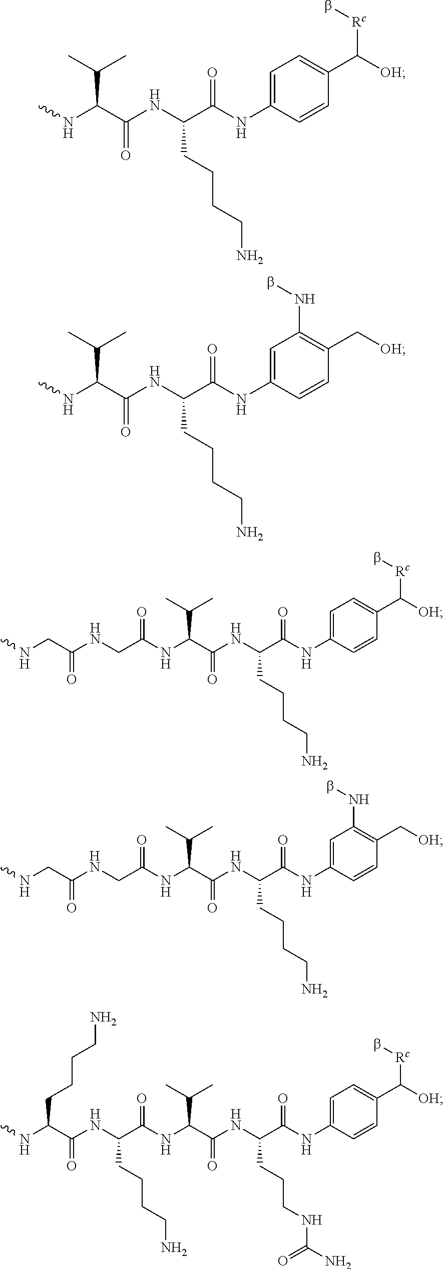

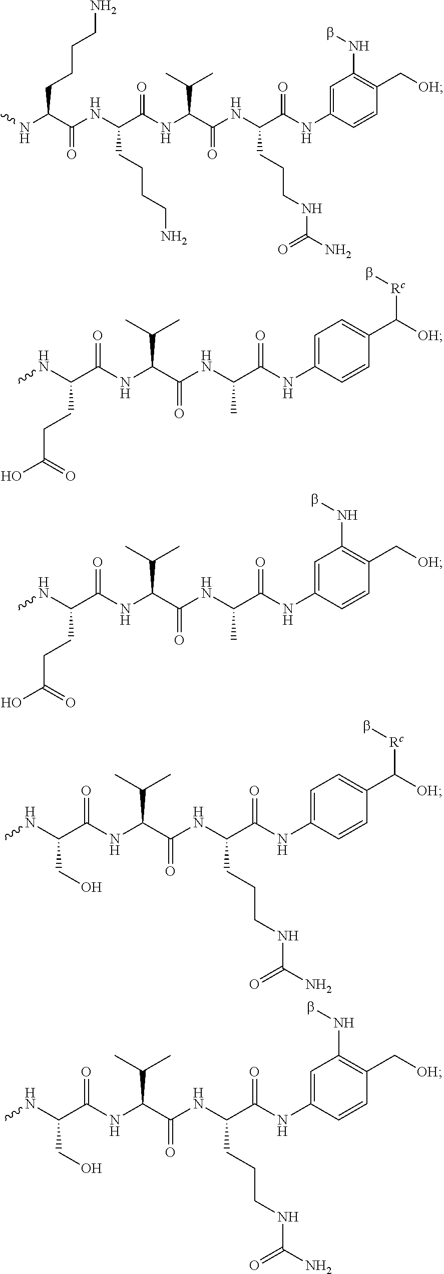

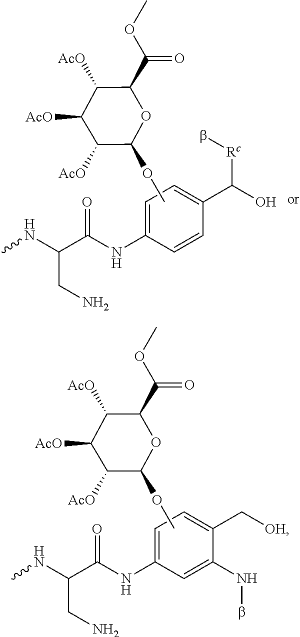

wherein
R^{c} is a bond or C₁₋₆ alkylene;
the wavy line on the amino group indicates an attachment site for a stretcher group or, prior to attachment to the stretcher group, indicates H;
β—is the attachment site to the at least one polar group; and
the benzylic H on the benzylic OH is optionally replaced with a bond to at least one of the the drug unit or to the linking group attached to at least one of the drug units.

Embodiment 34. The linker compound of any of Embodiments 1-33, wherein the enzyme-cleavable group comprises a peptide that is cleavable by an intracellular protease.

Embodiment 35. The linker compound of Embodiment 34, wherein the intracellular protease is Cathepsin B.

Embodiment 36. The linker compound of Embodiment 34, wherein the enzyme-cleavable group comprises a cleavable peptide including a valine-citrulline peptide, a valine-alanine peptide, a valine-lysine peptide, a phenylalanine-lysine peptide, or a glycine-glycine-phenylalanine-glycine peptide.

Embodiment 37. The linker compound of any of Embodiments 1-36, comprising one of the following structures:

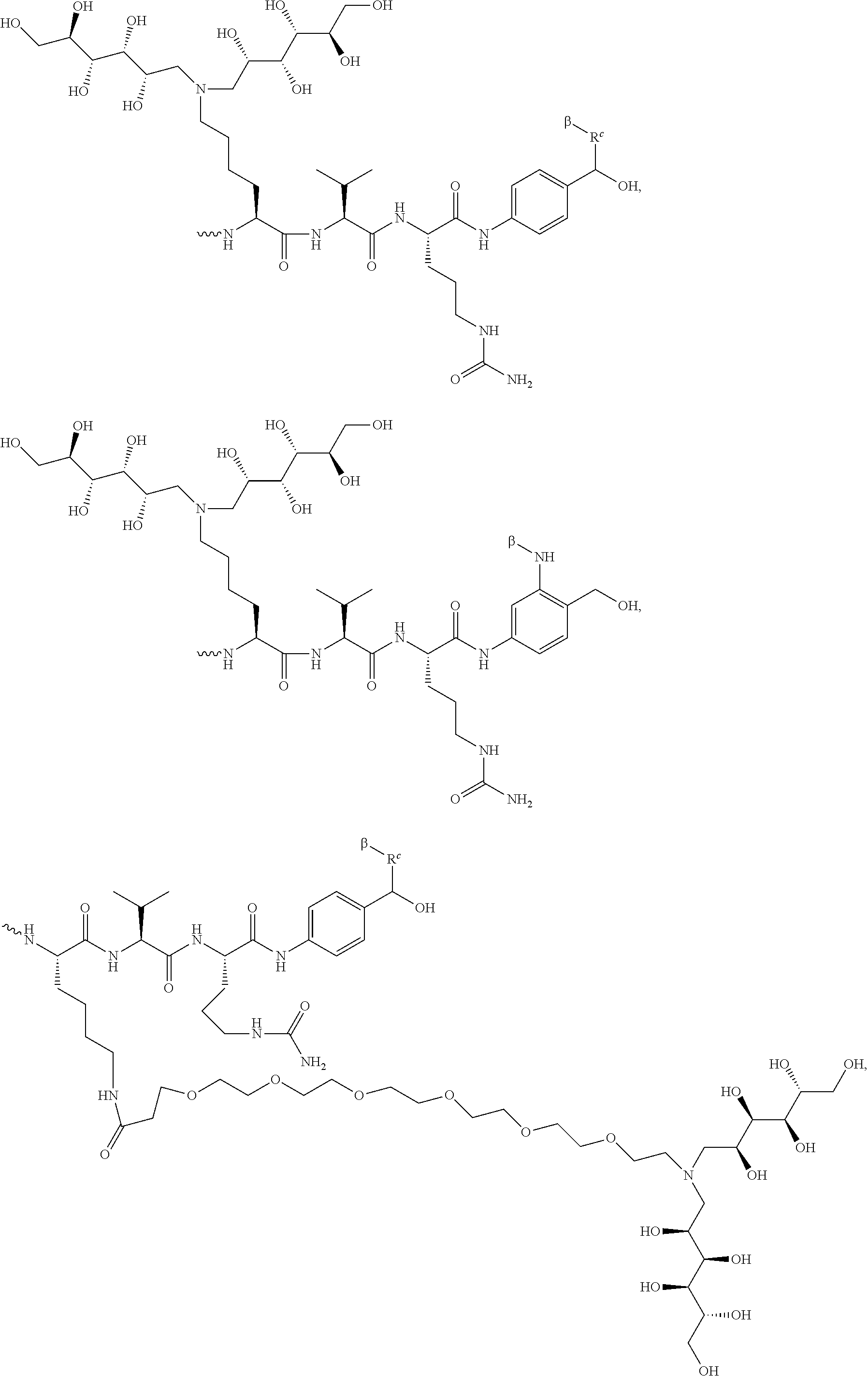

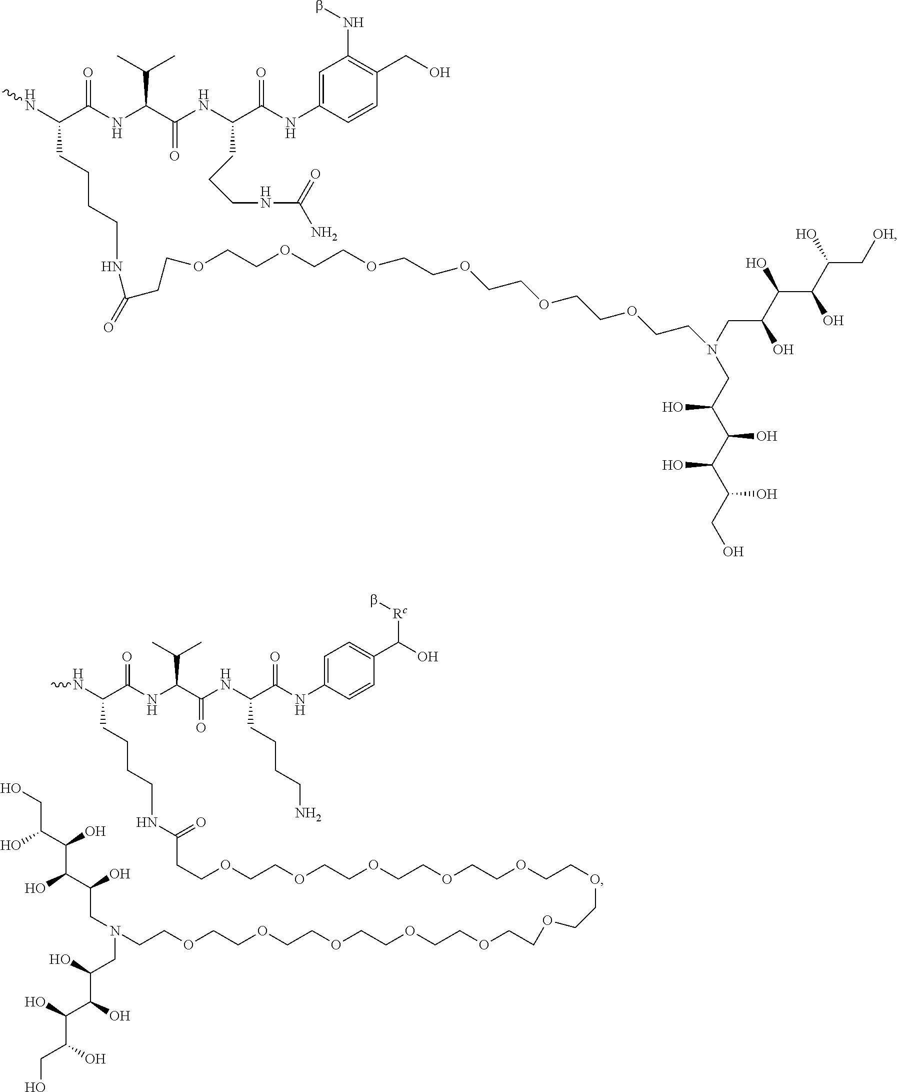

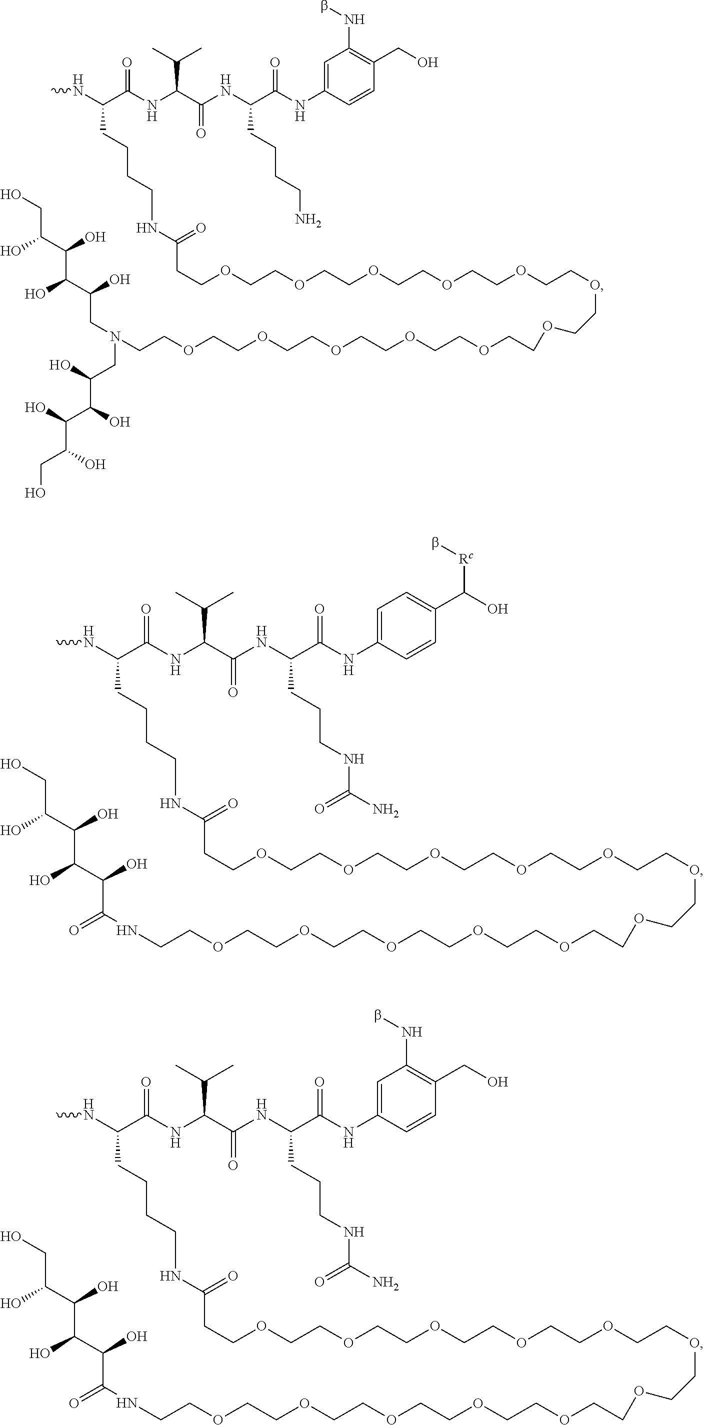

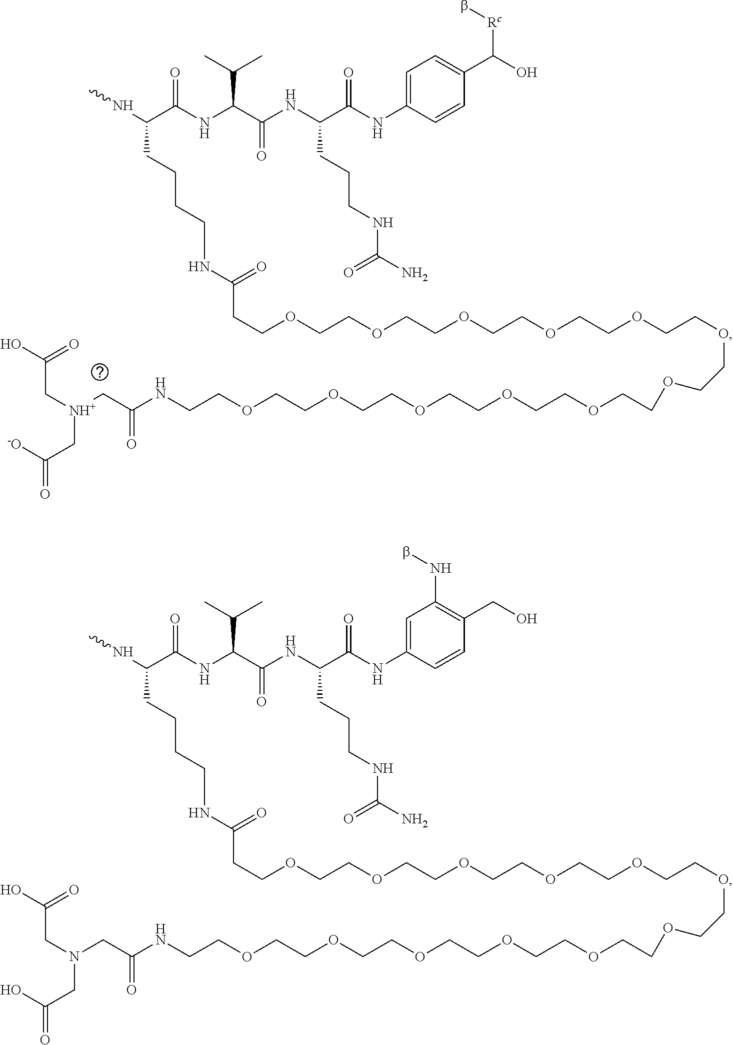

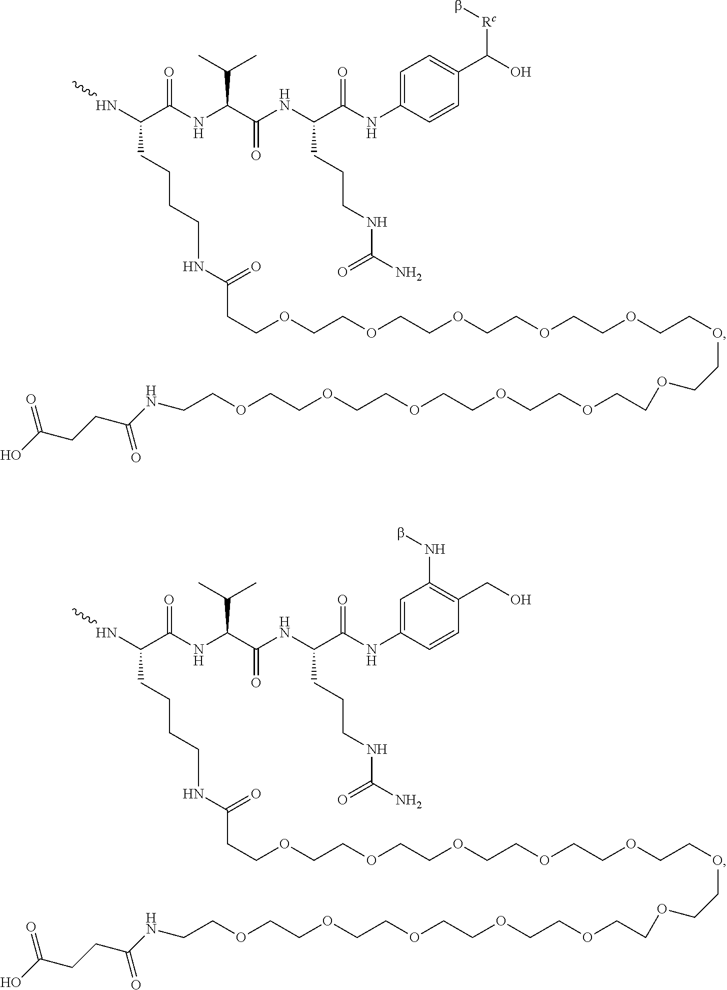

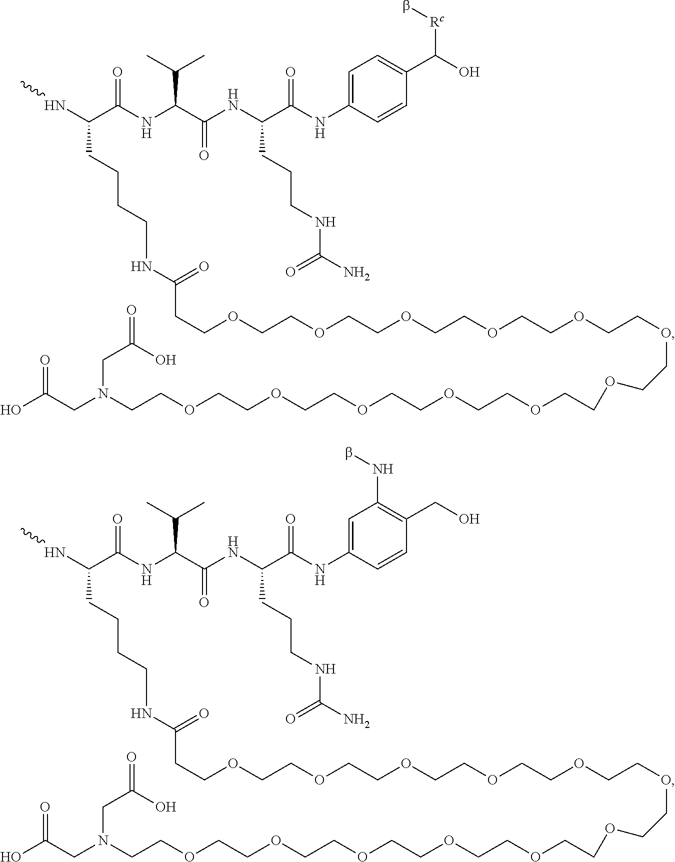

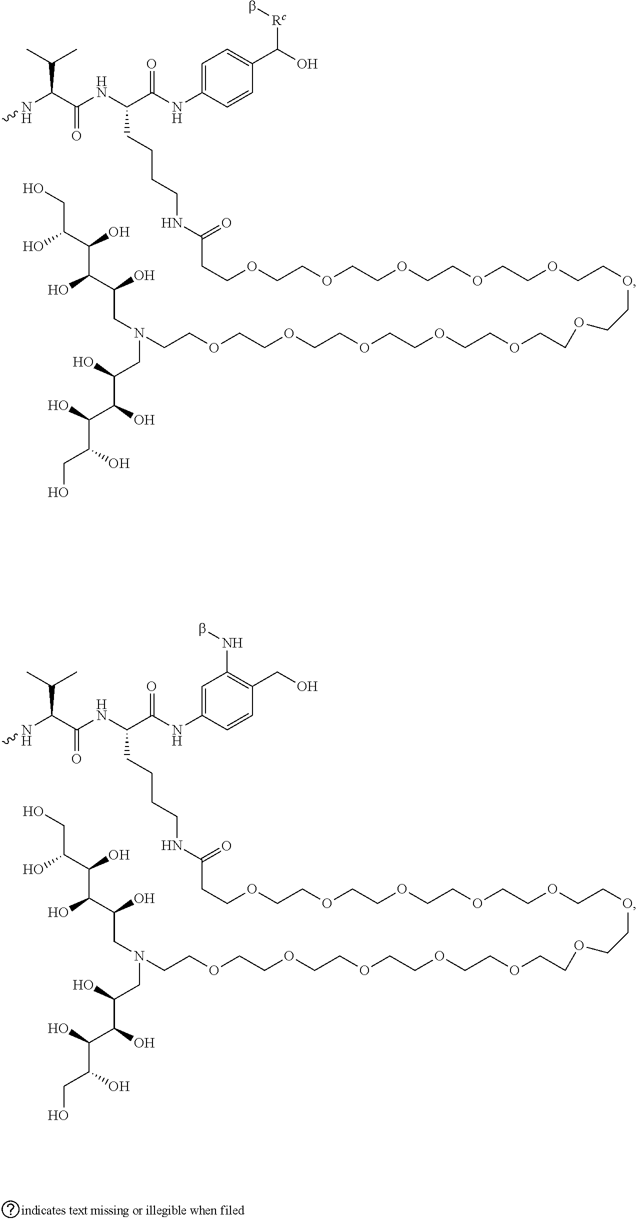

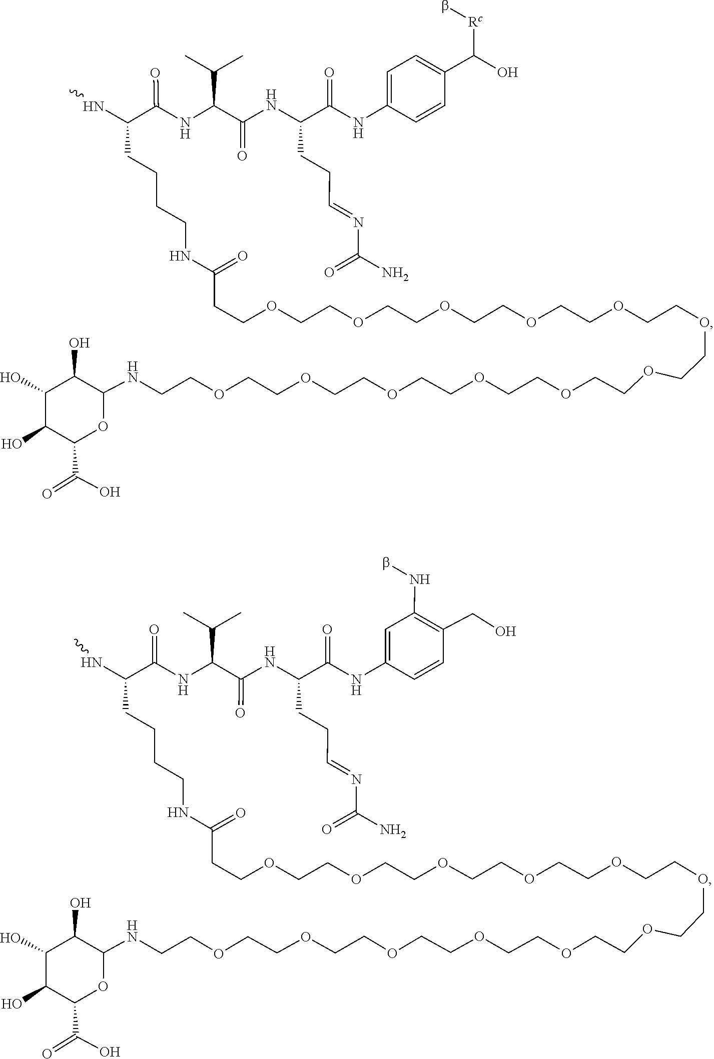

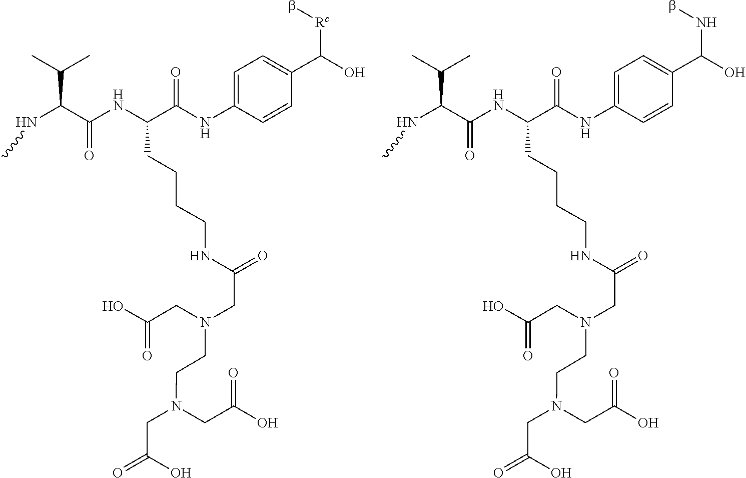

wherein
R^{c} is a bond or C₁₋₆ alkylene;
the wavy line on the amino group indicates an attachment site for the stretcher group or, prior to attachment to the stretcher group, indicates H;
β—is an attachment site to a POLY unit; and
the H on the benzylic OH is optionally replaced with a bond to at least one of the drug units or to the attachment site to at least one of the drug units.

Embodiment 38. The linker compound of any of Embodiments 1-4, having one of the following structures:

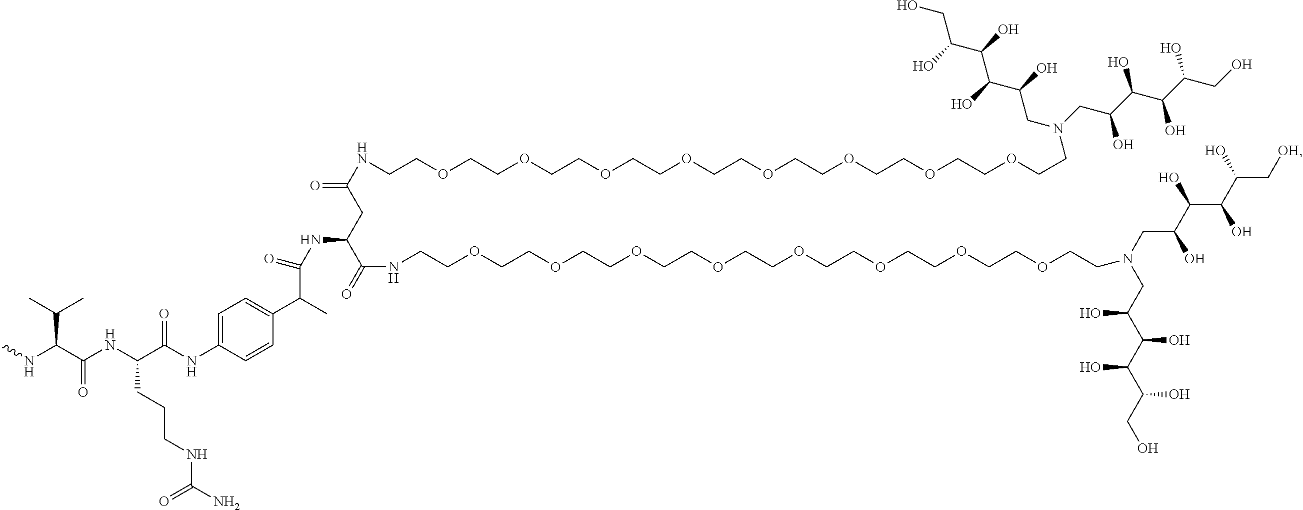

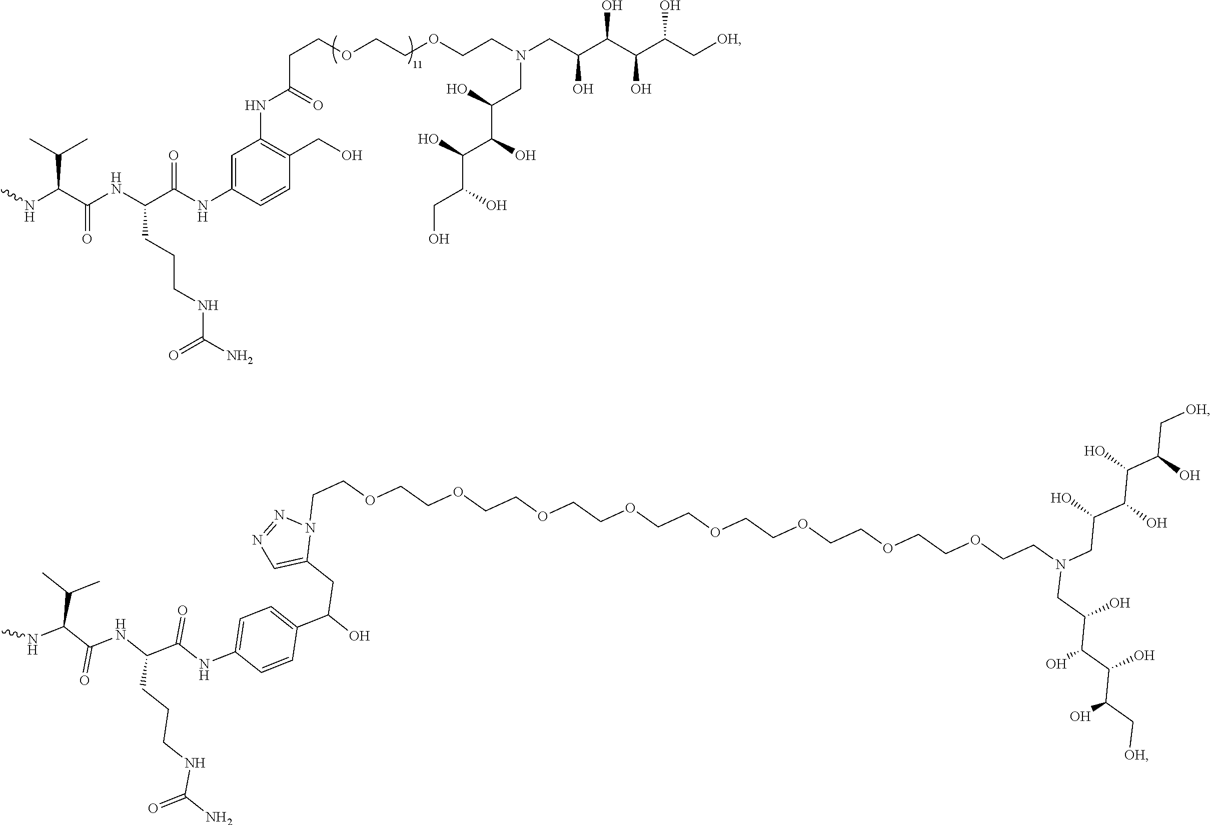

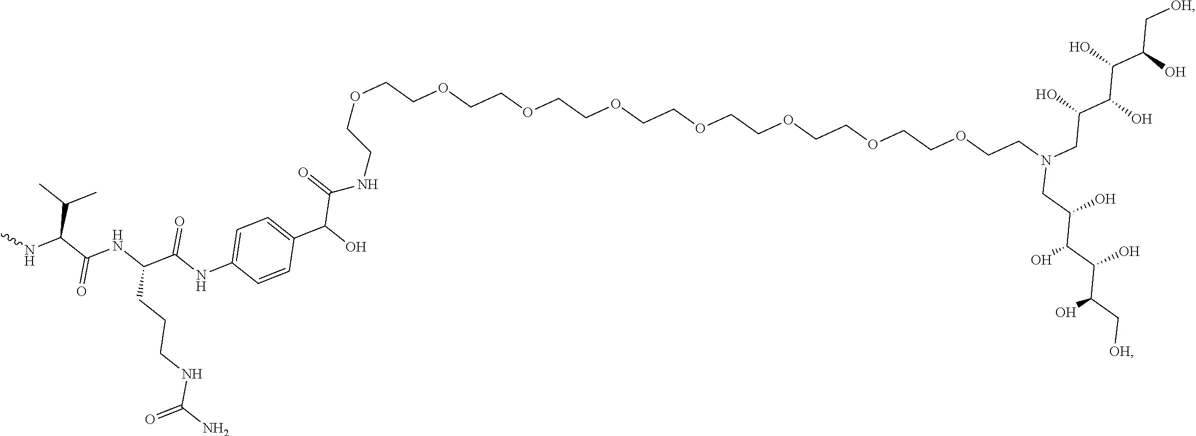

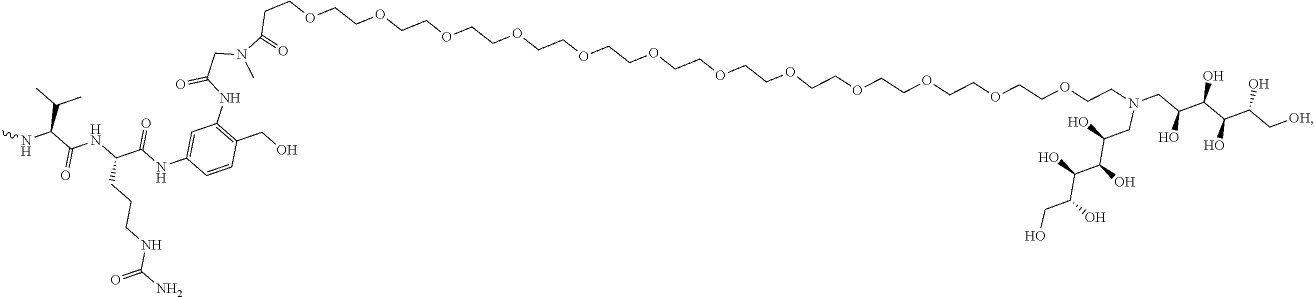

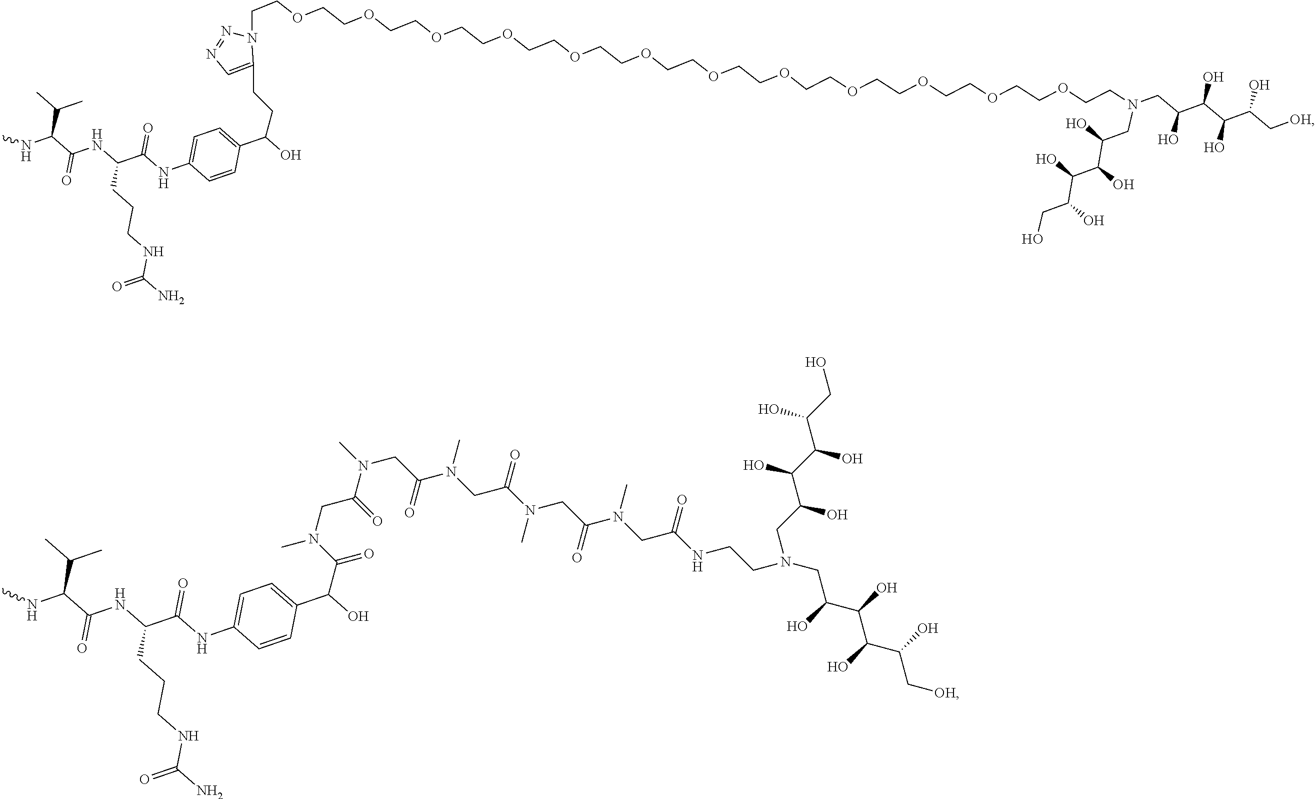

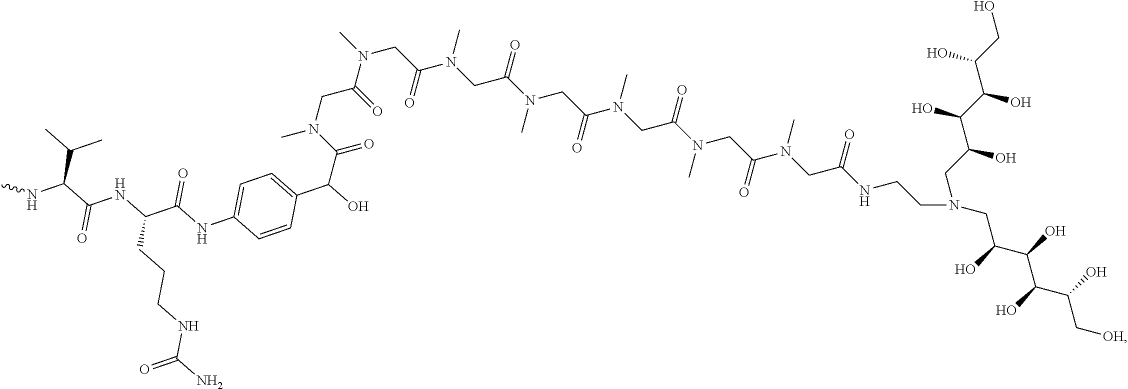

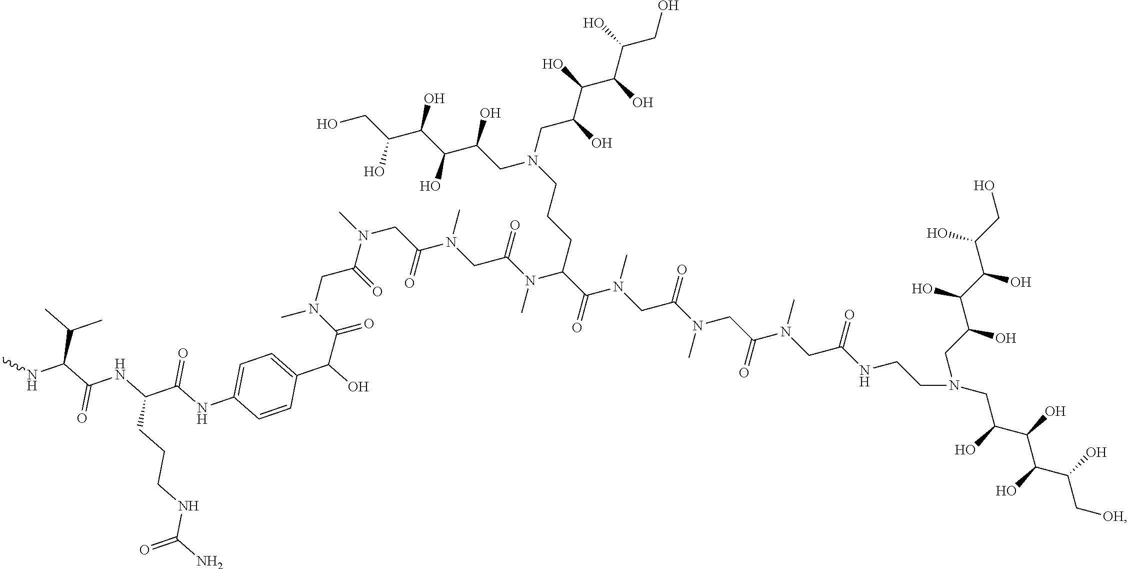

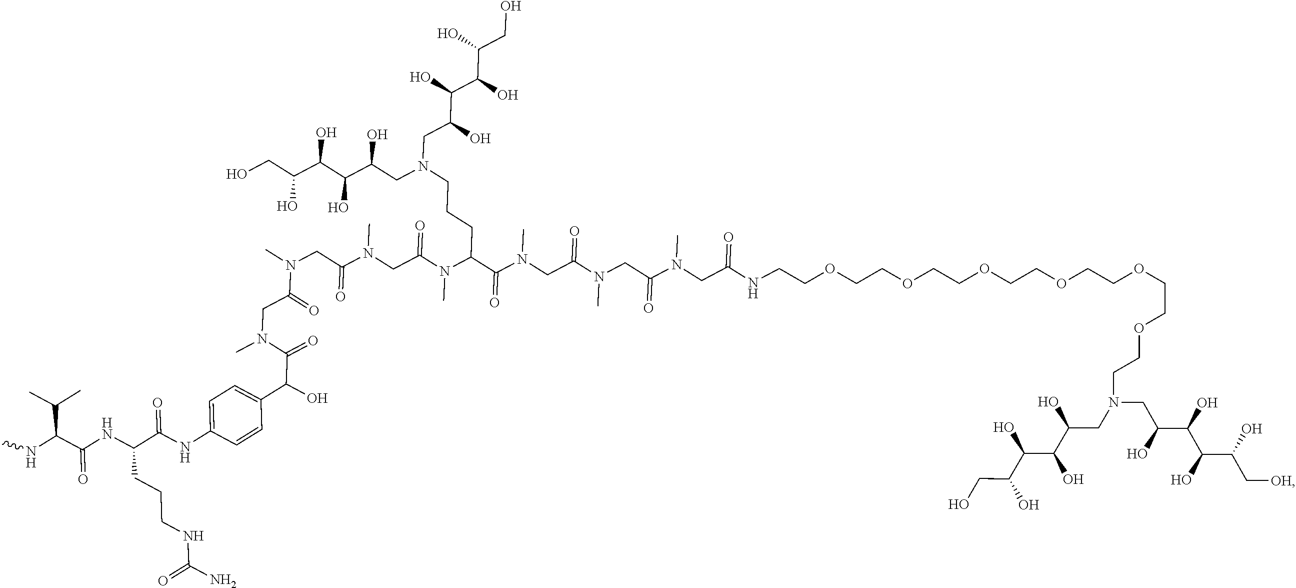

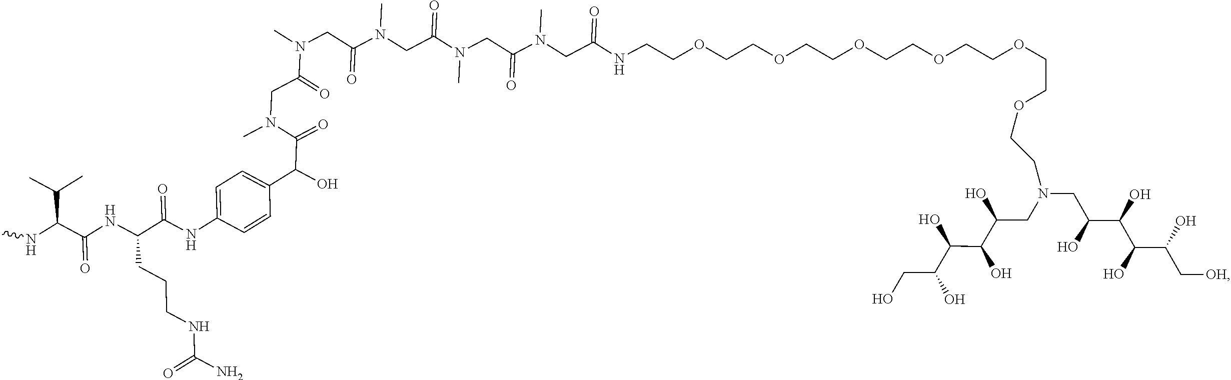

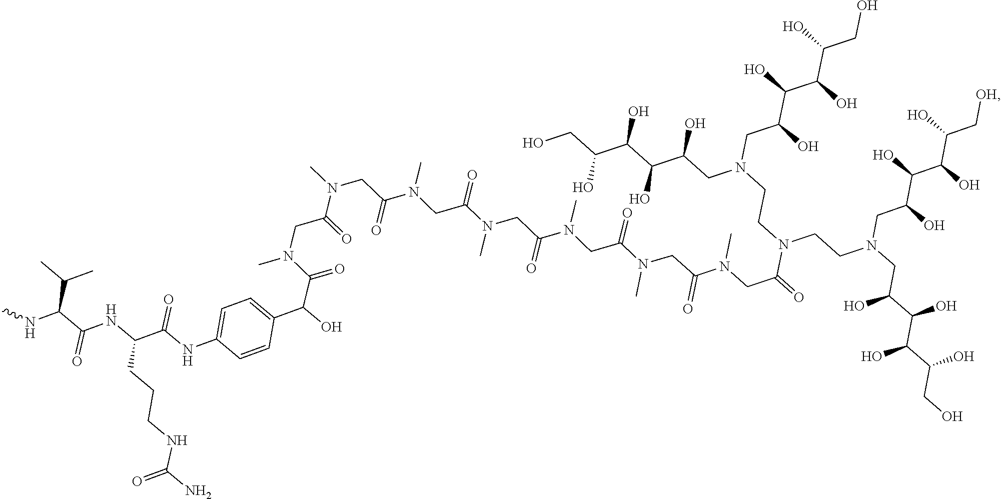

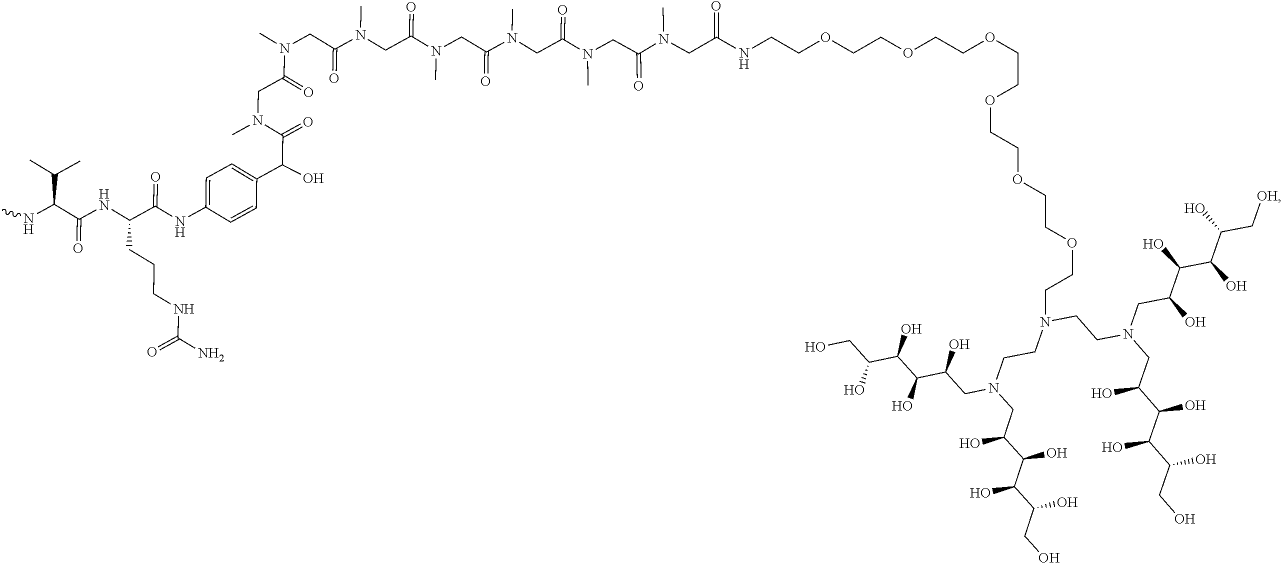

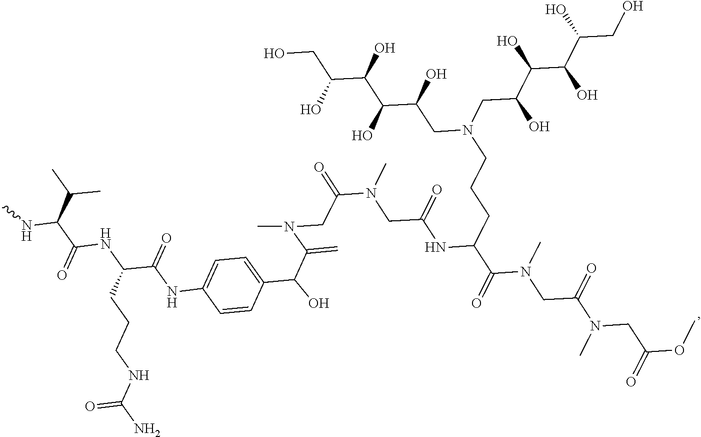

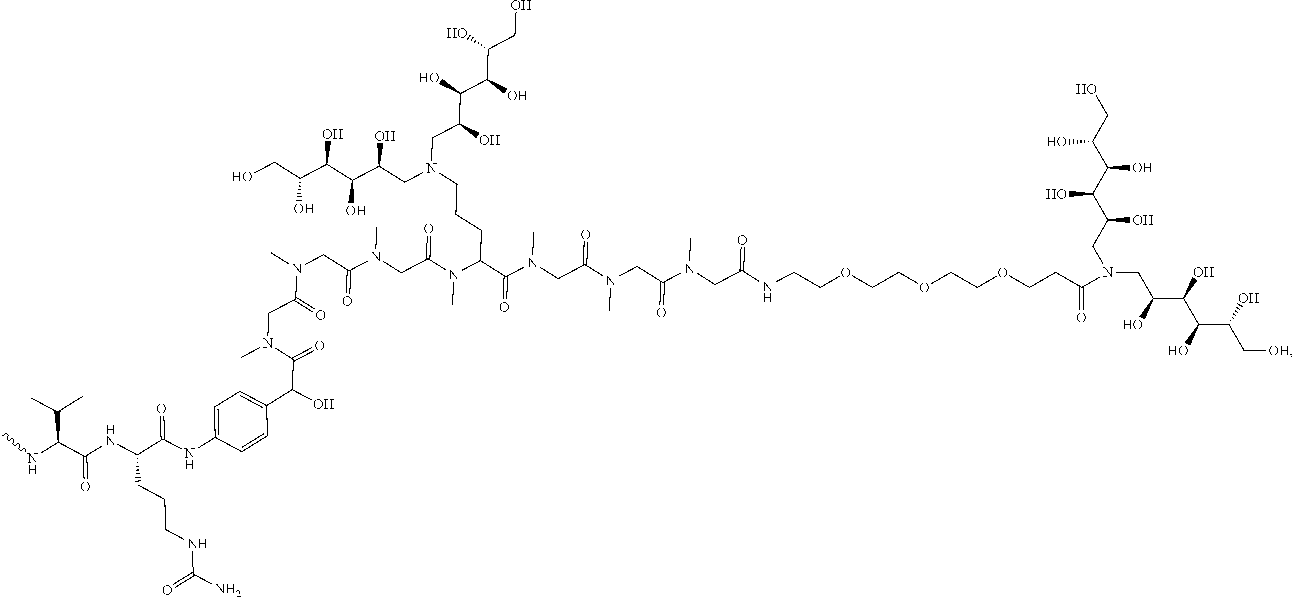

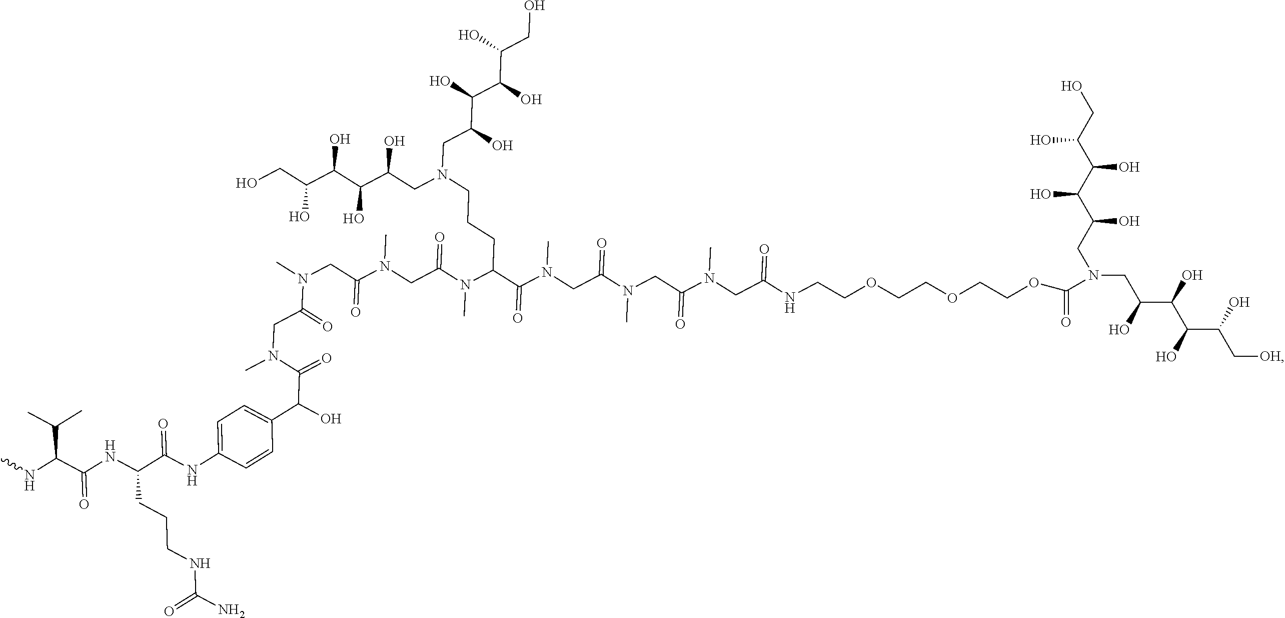

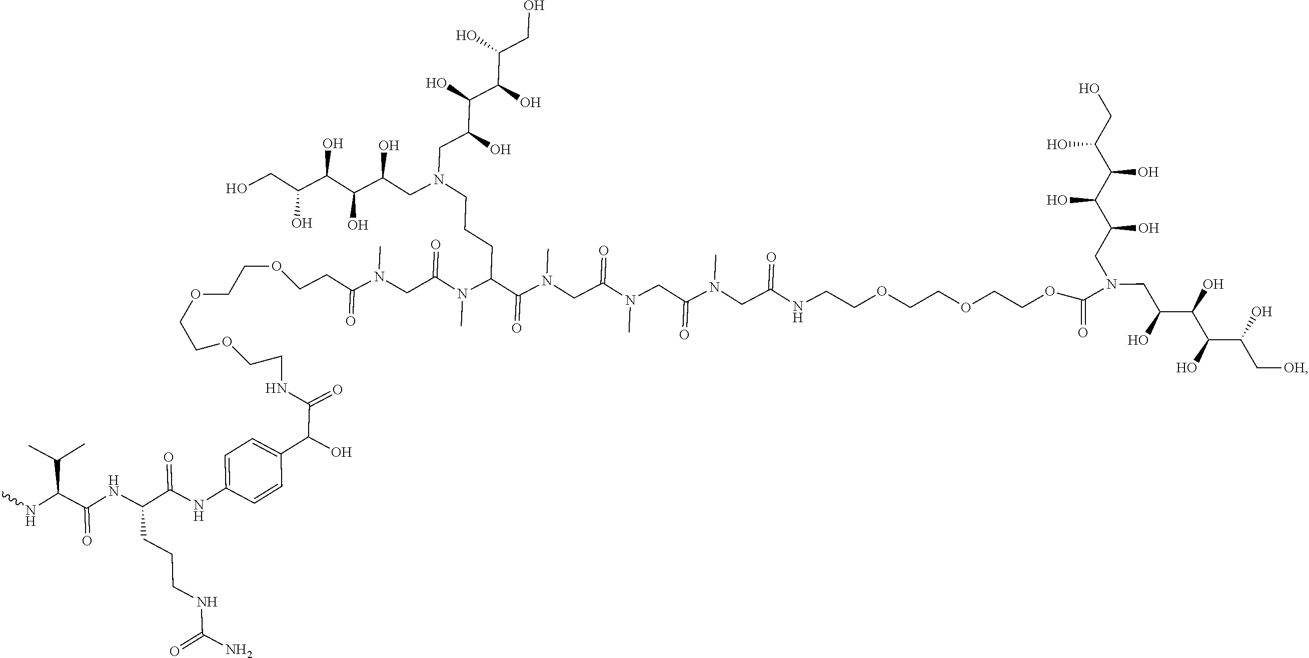

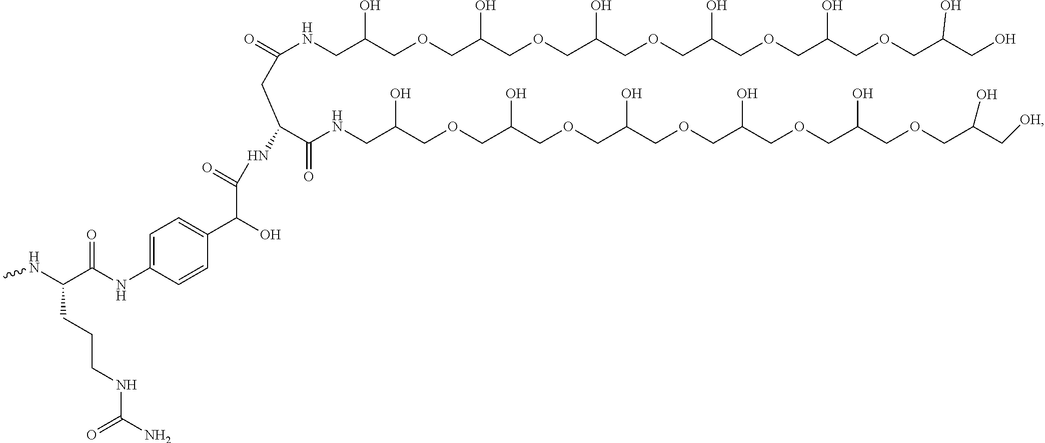

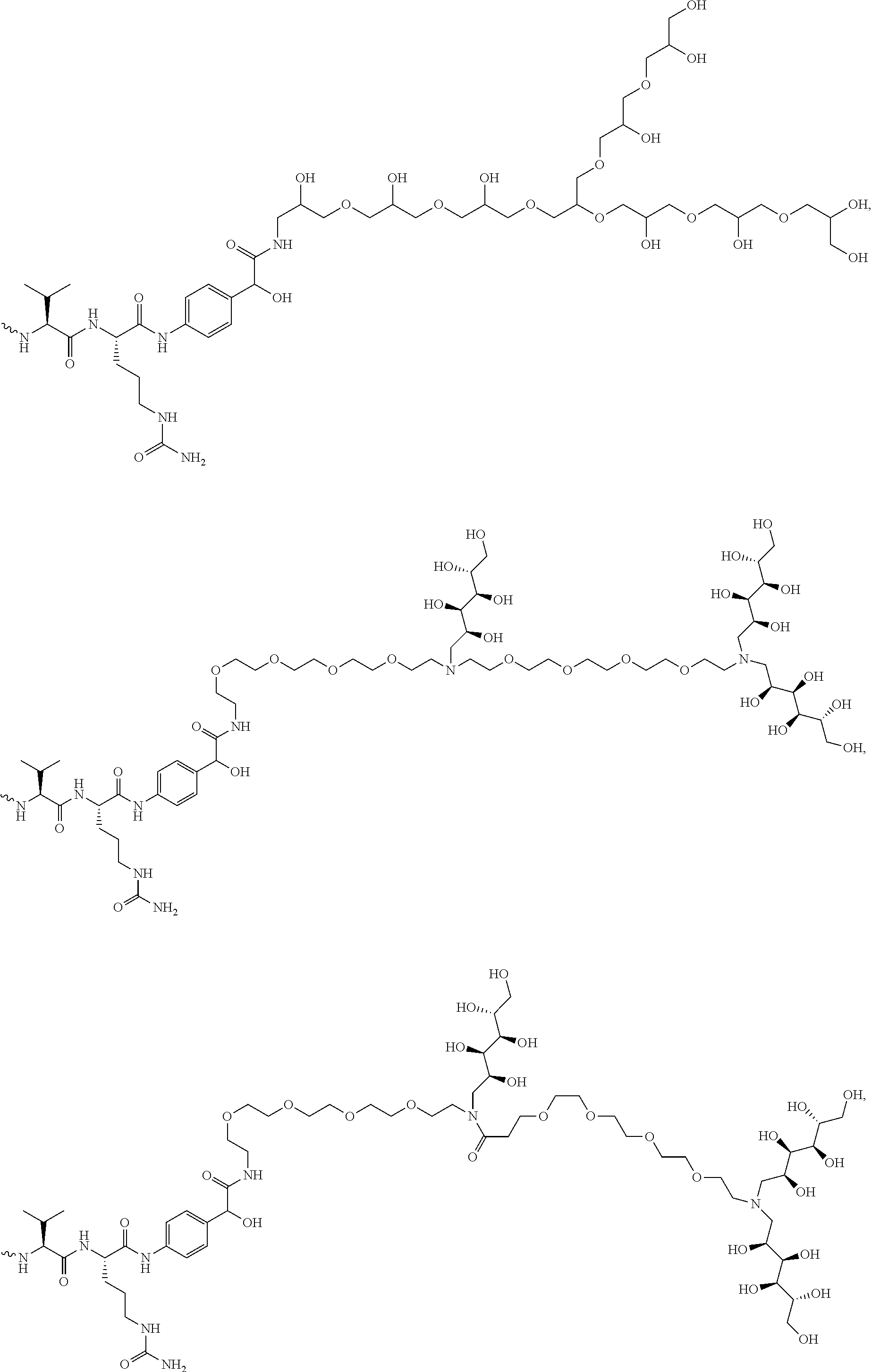

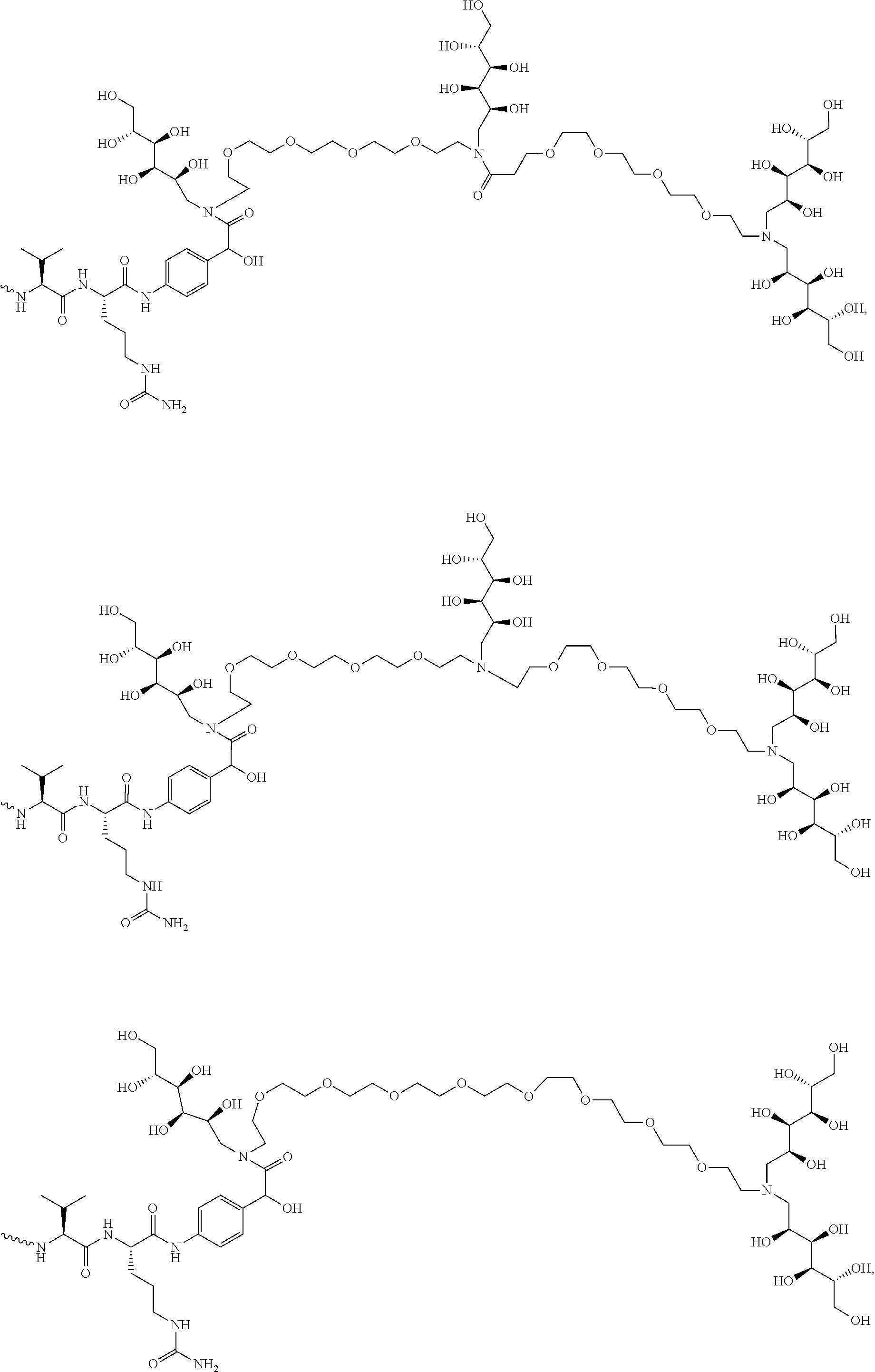

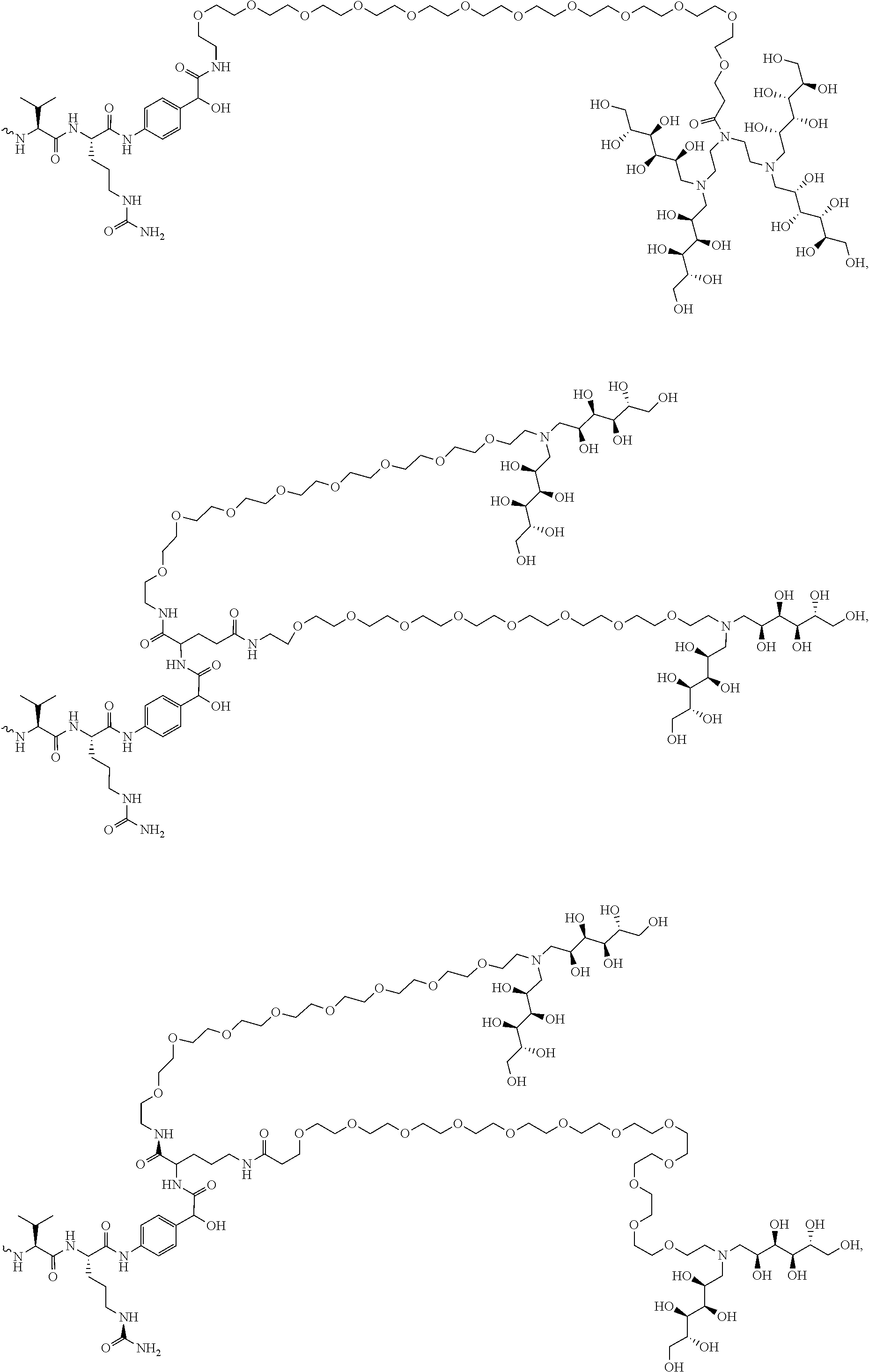

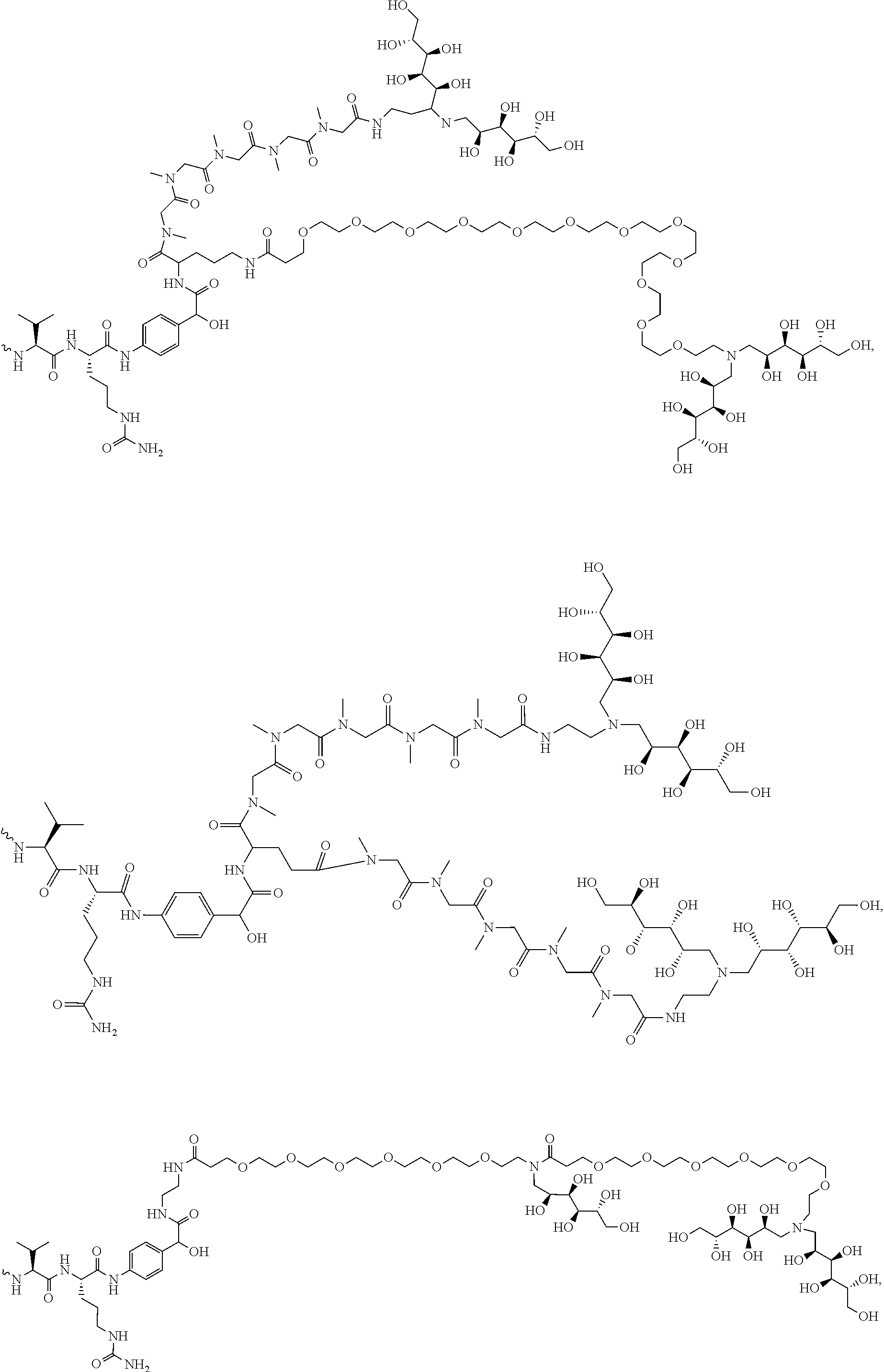

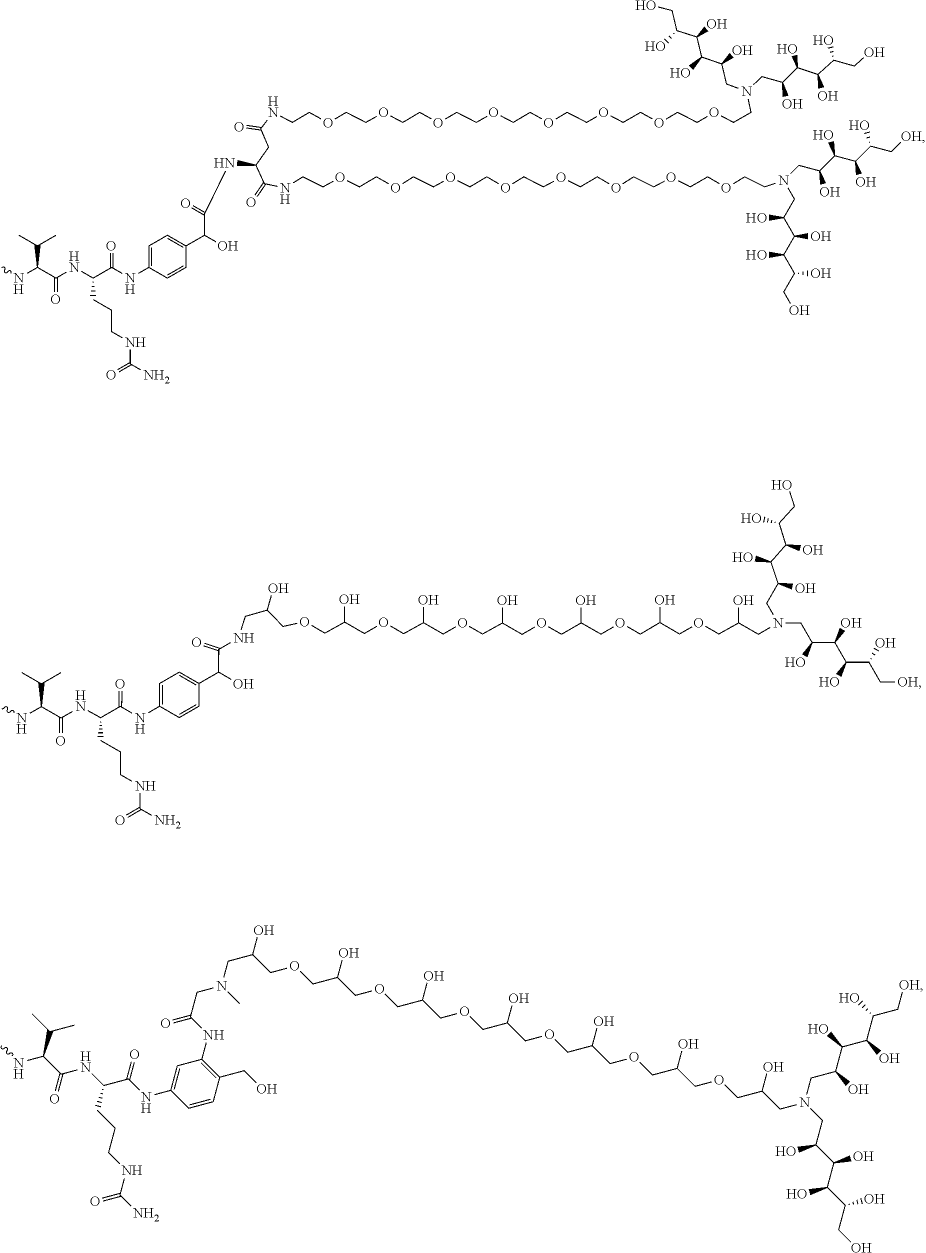

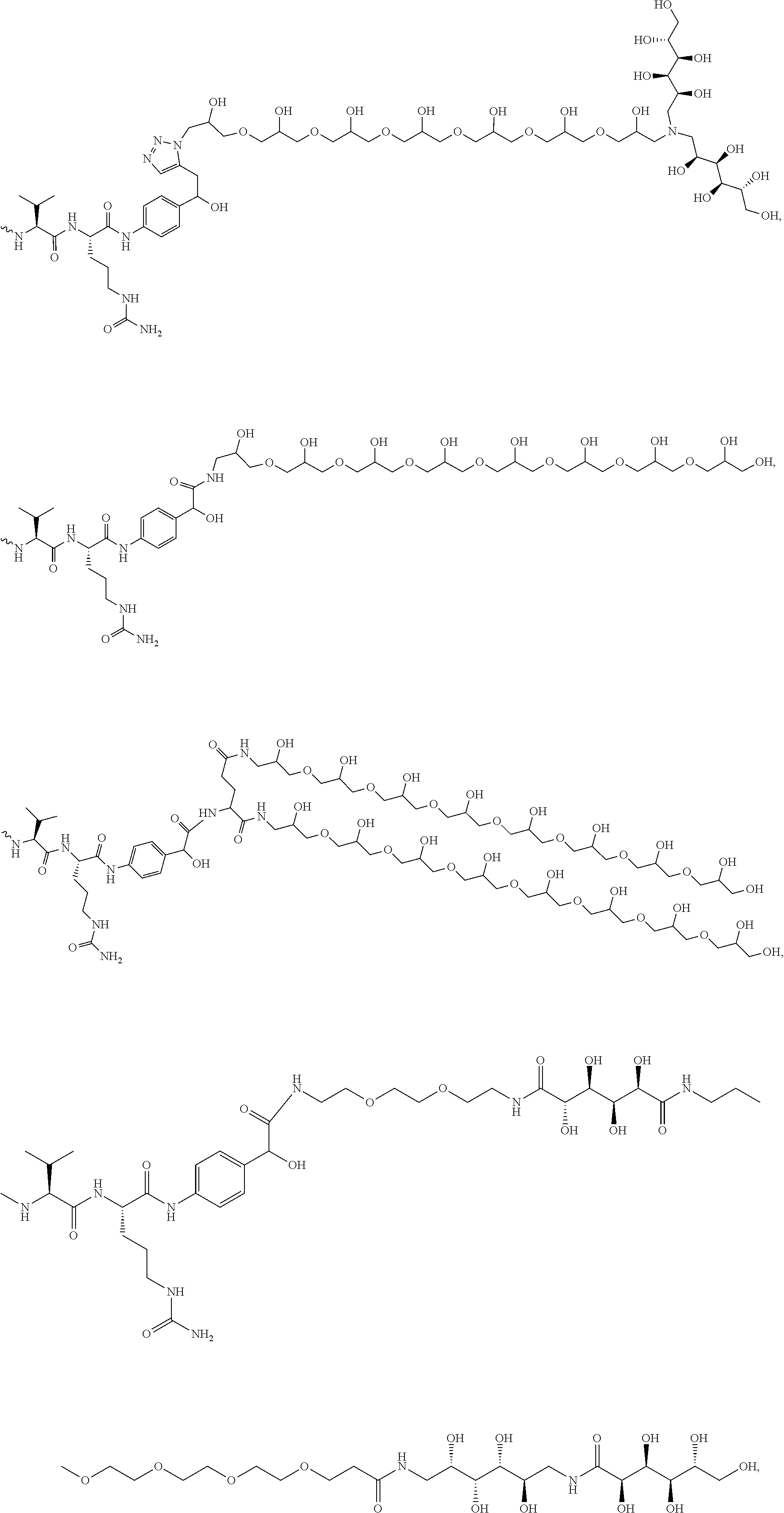

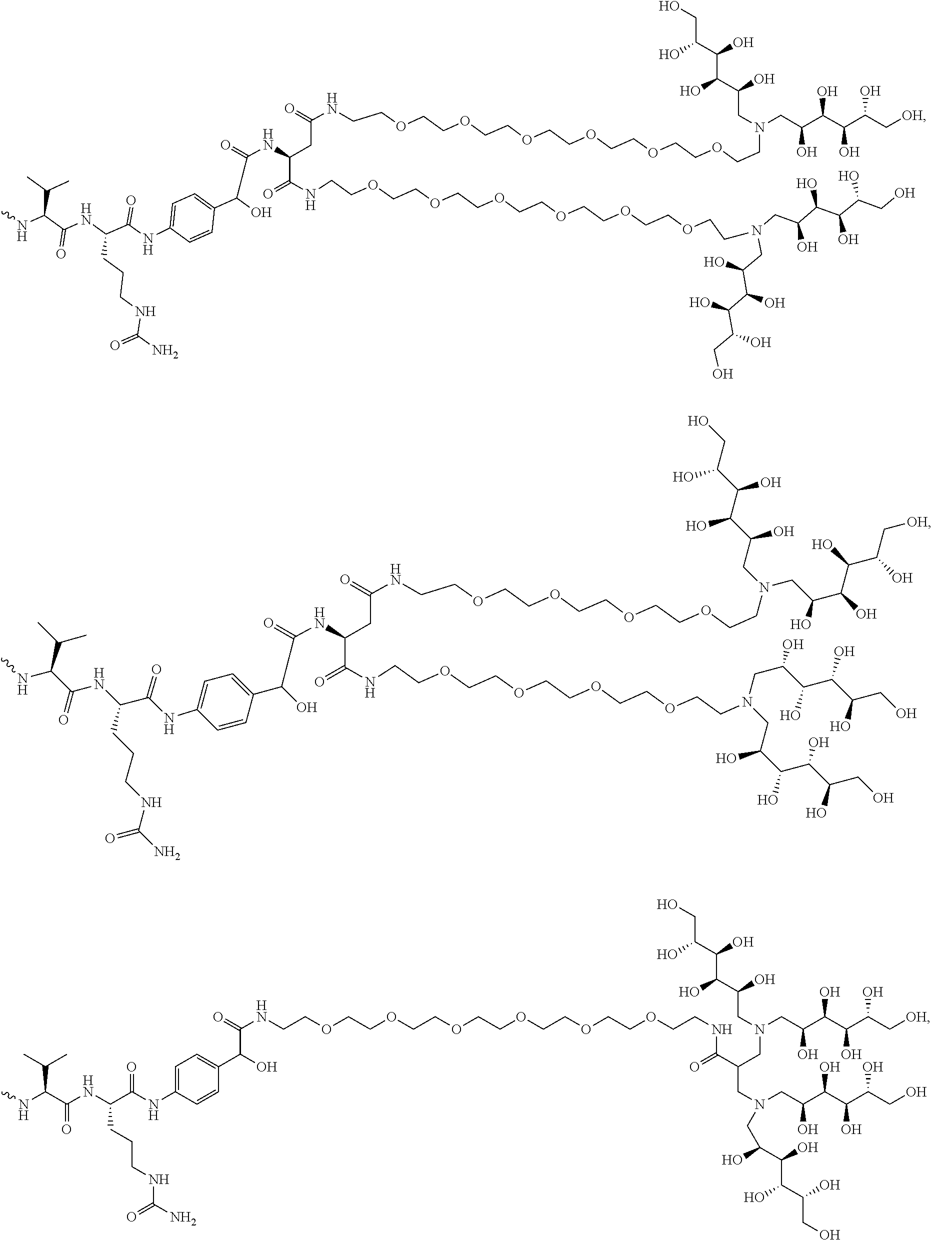

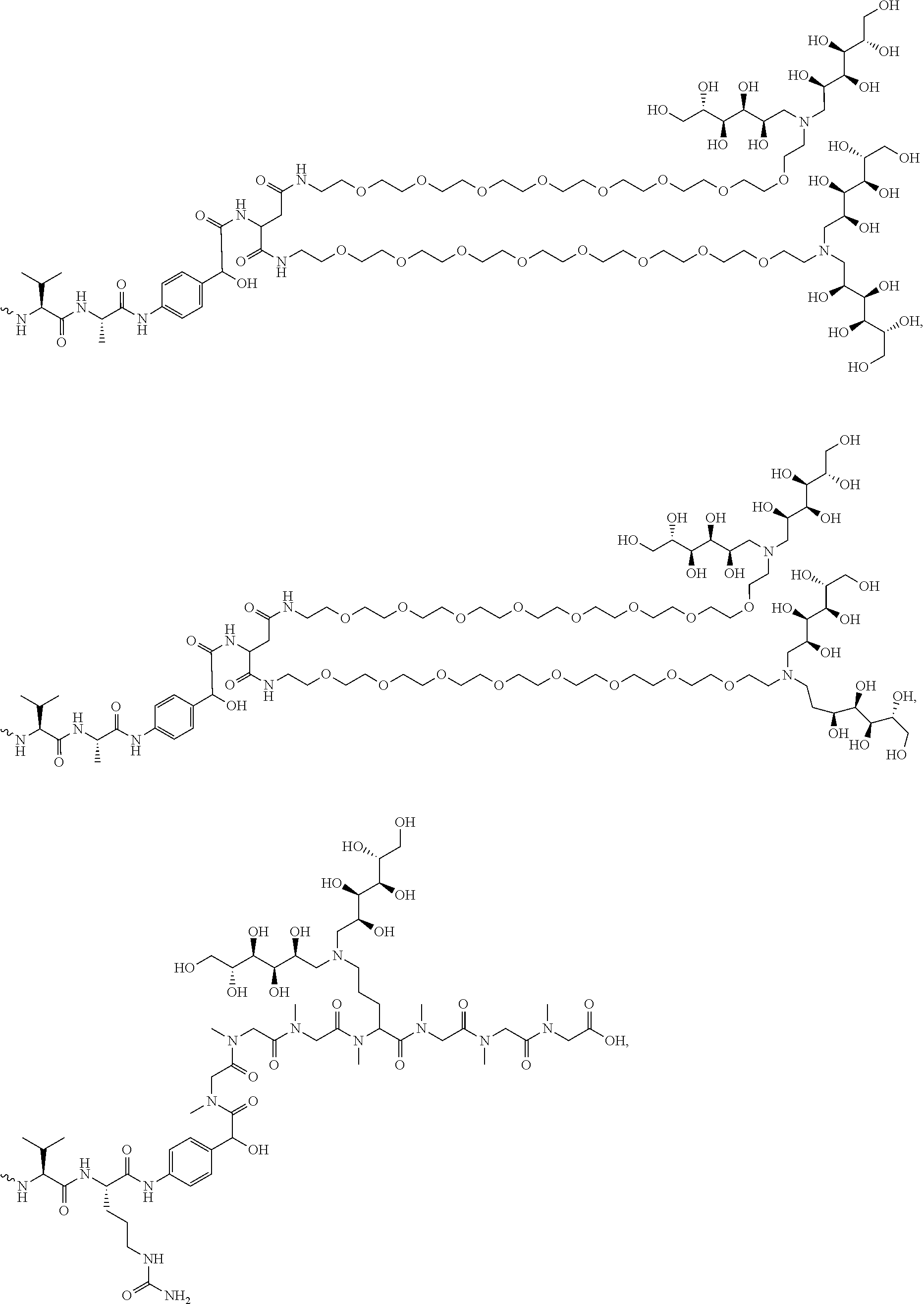

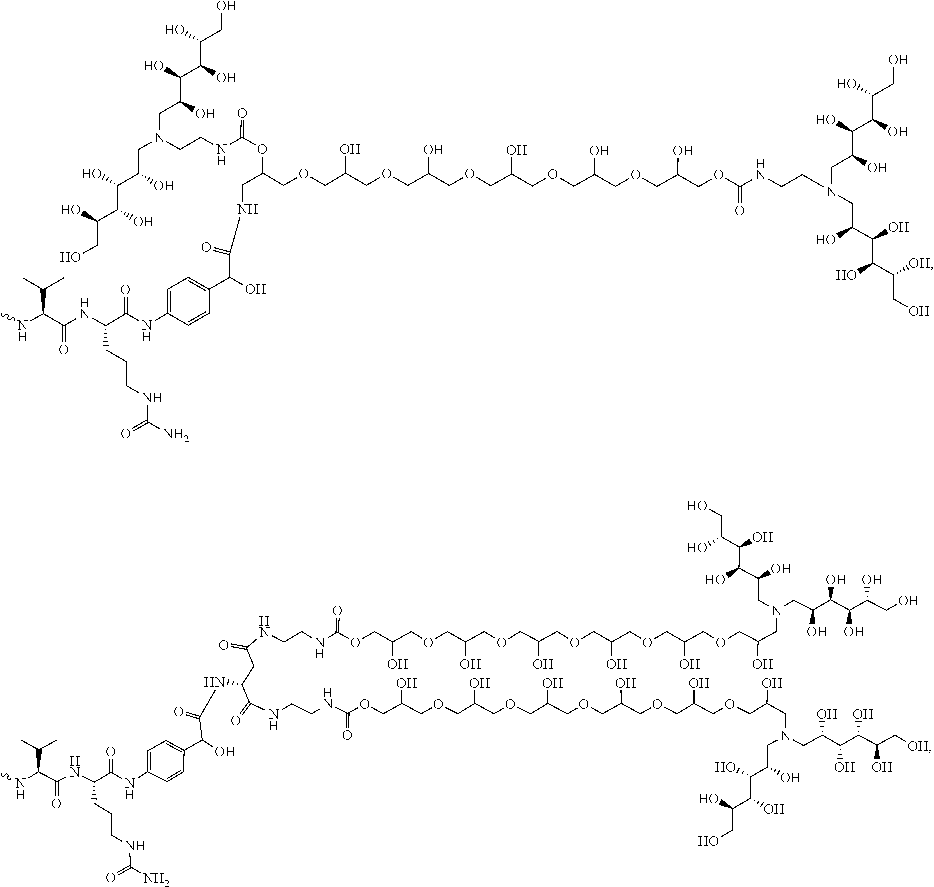

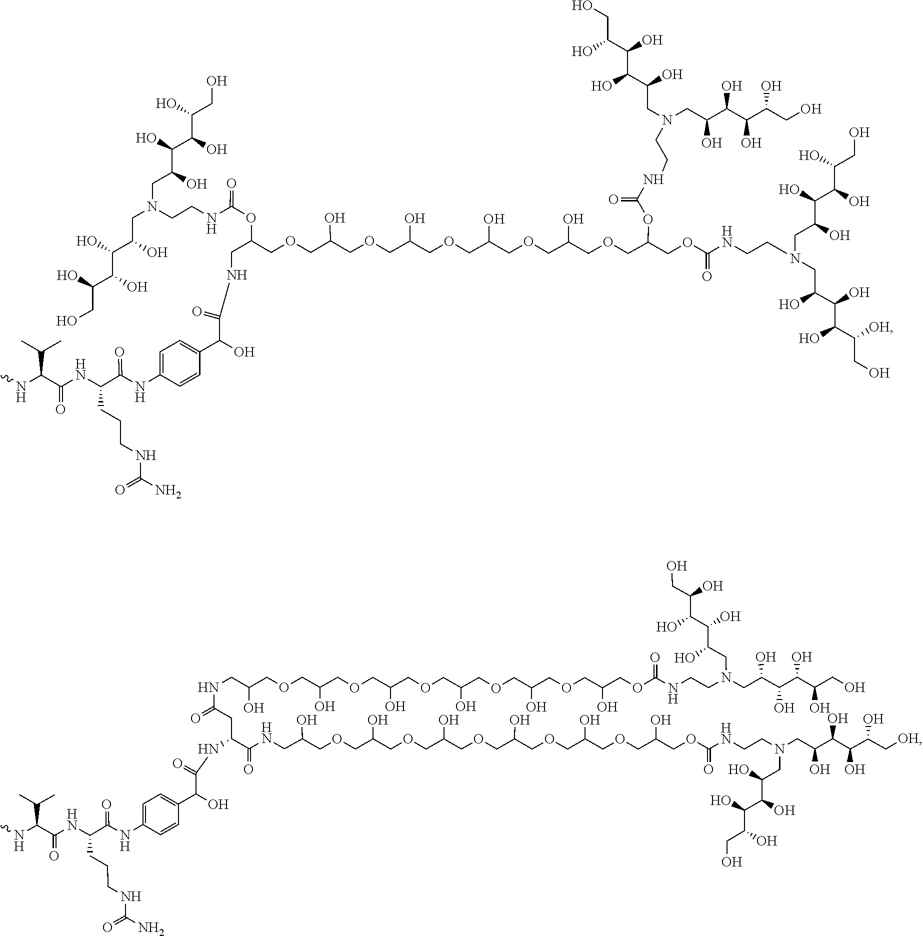

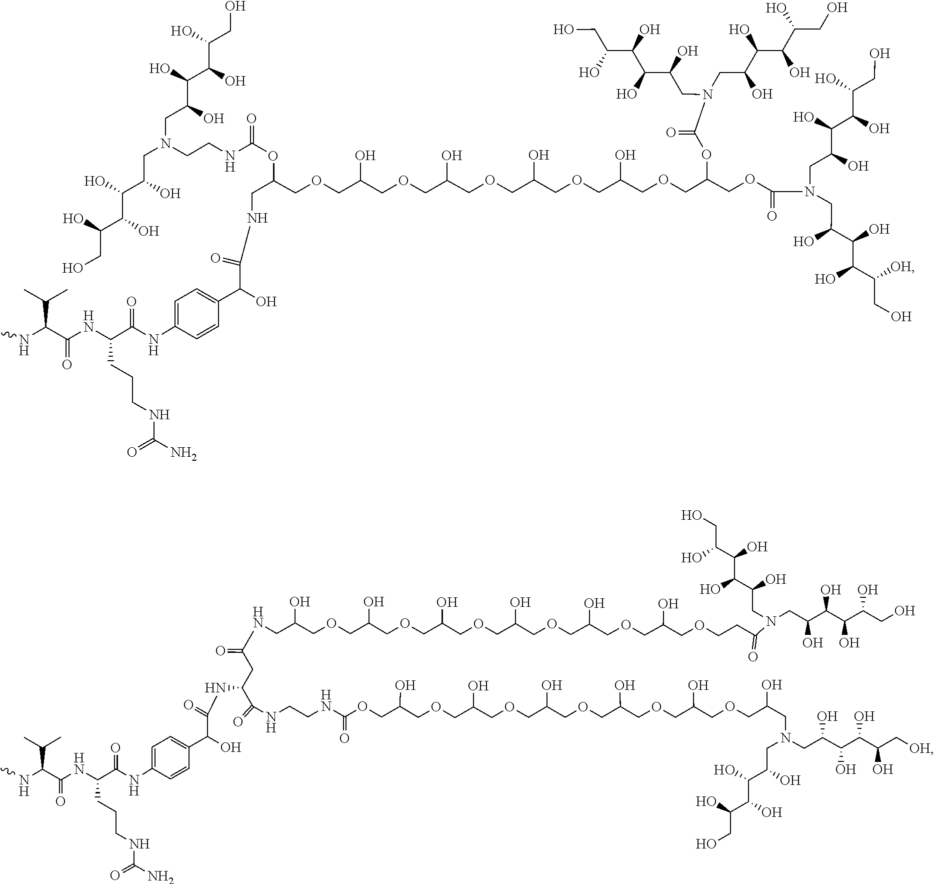

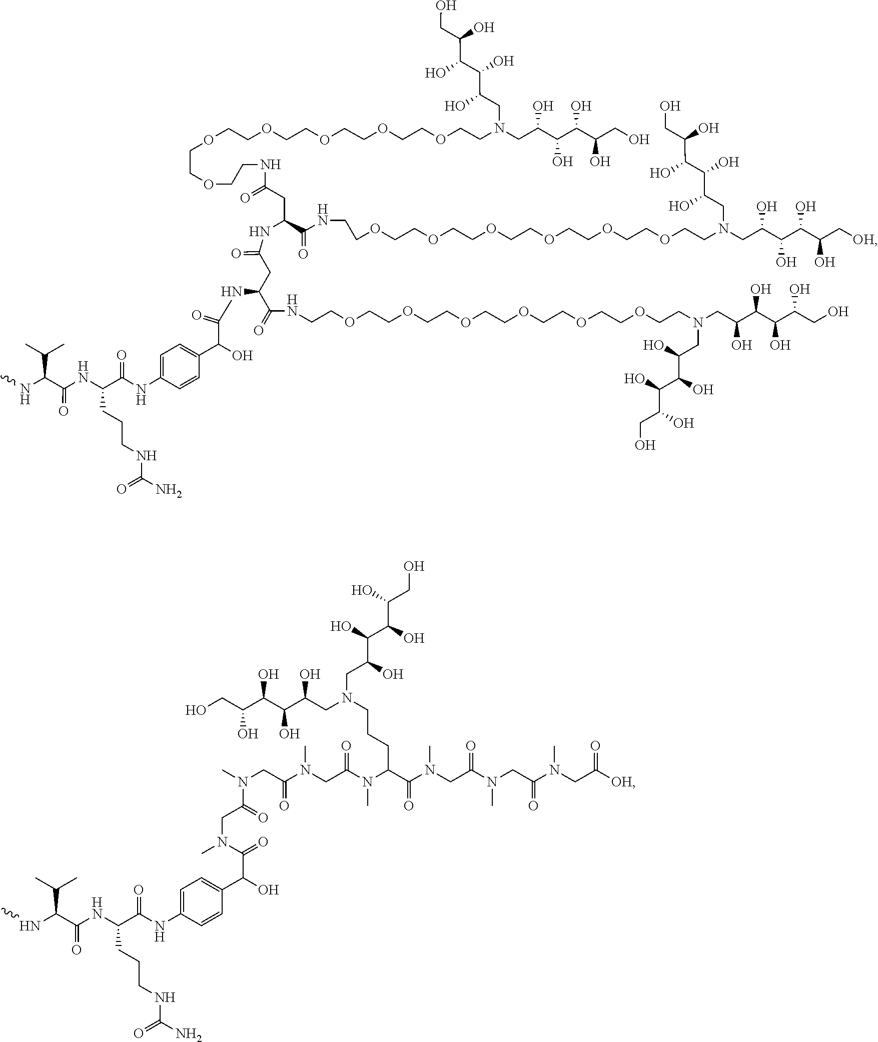

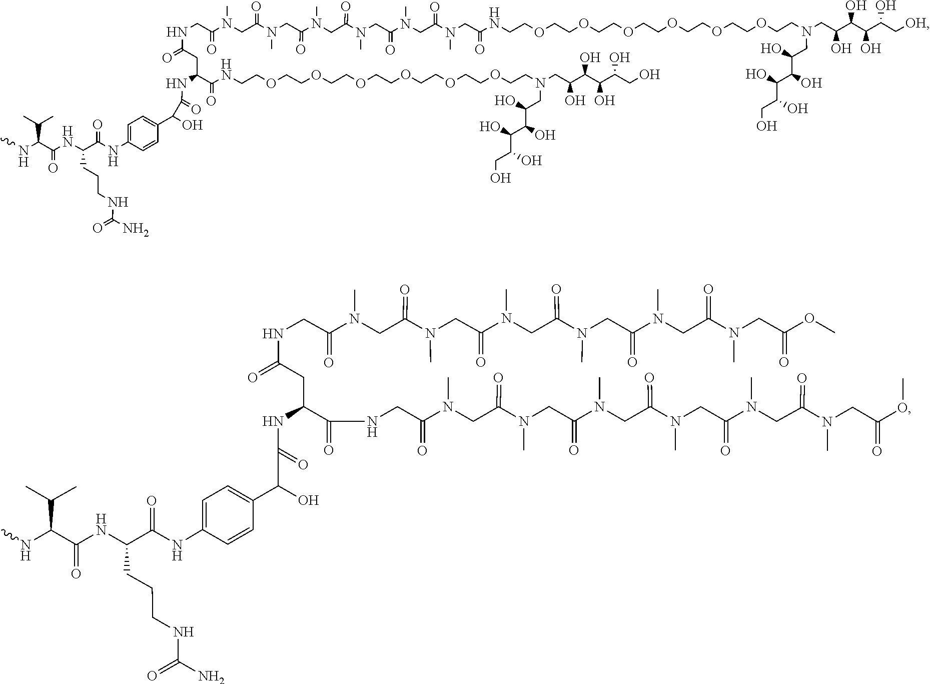

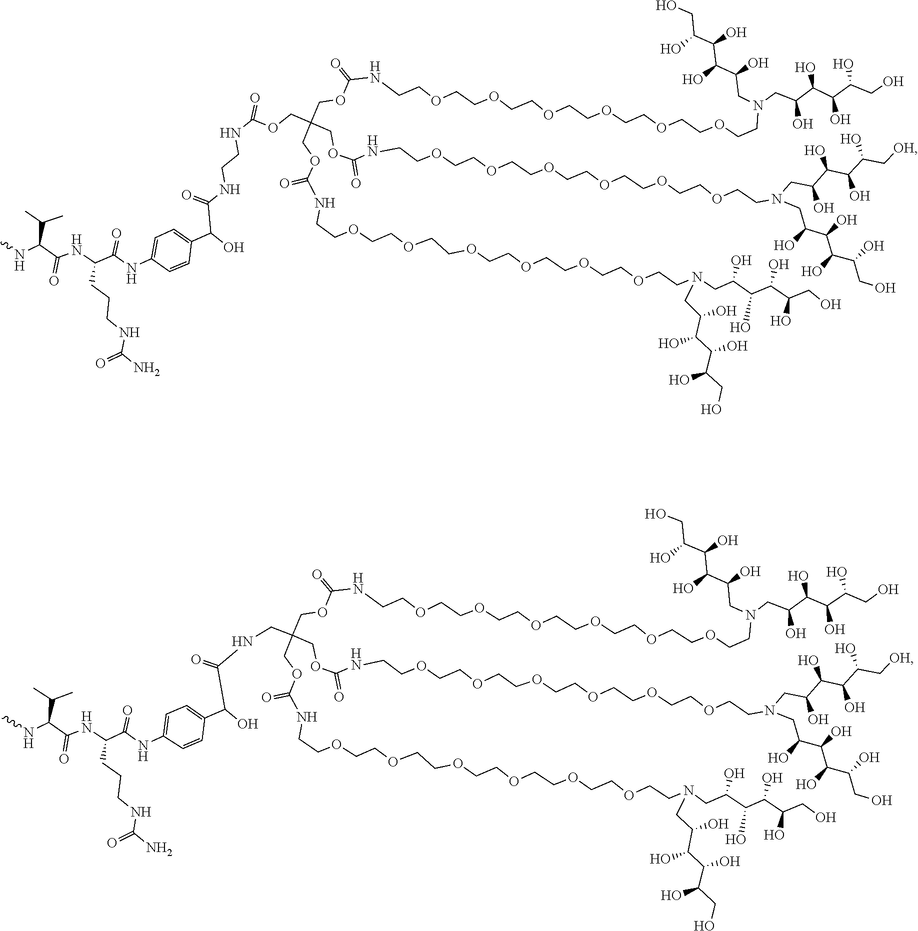

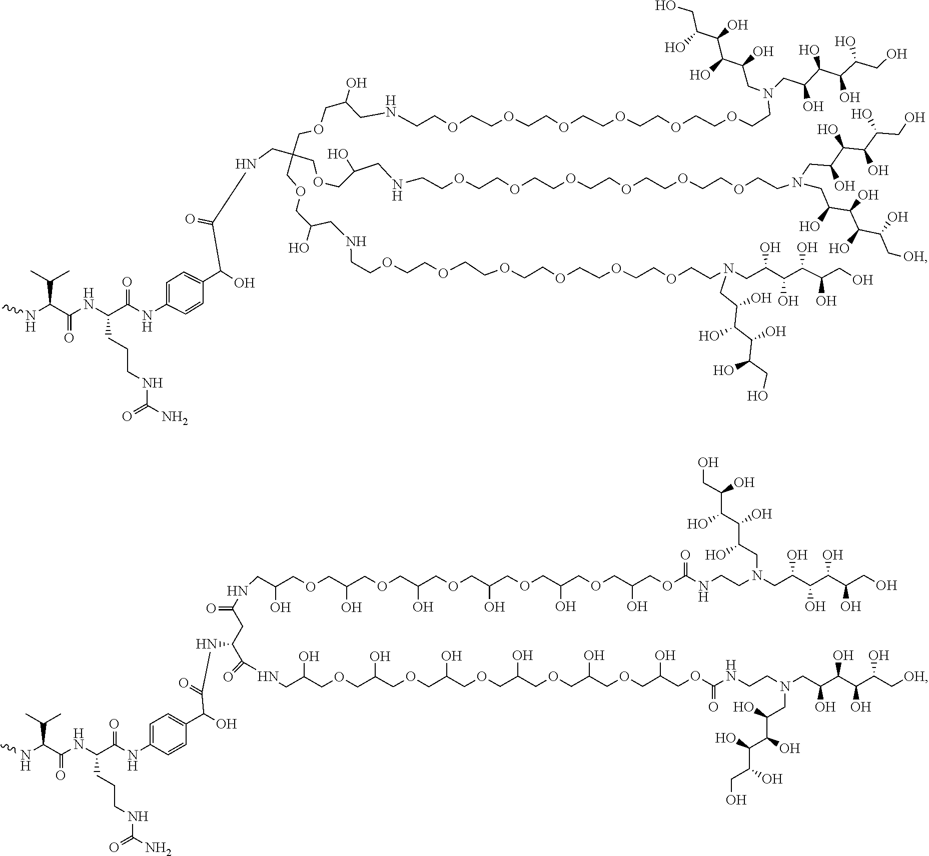

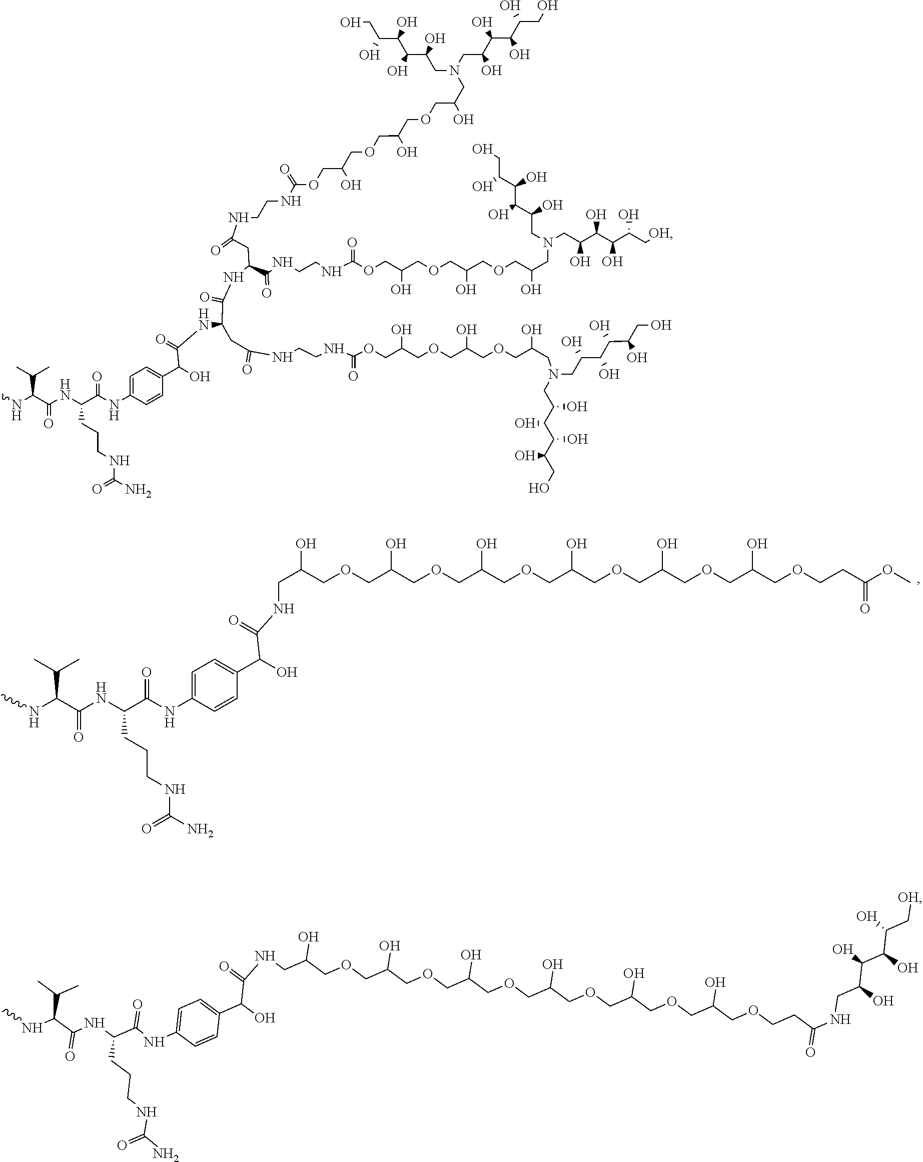

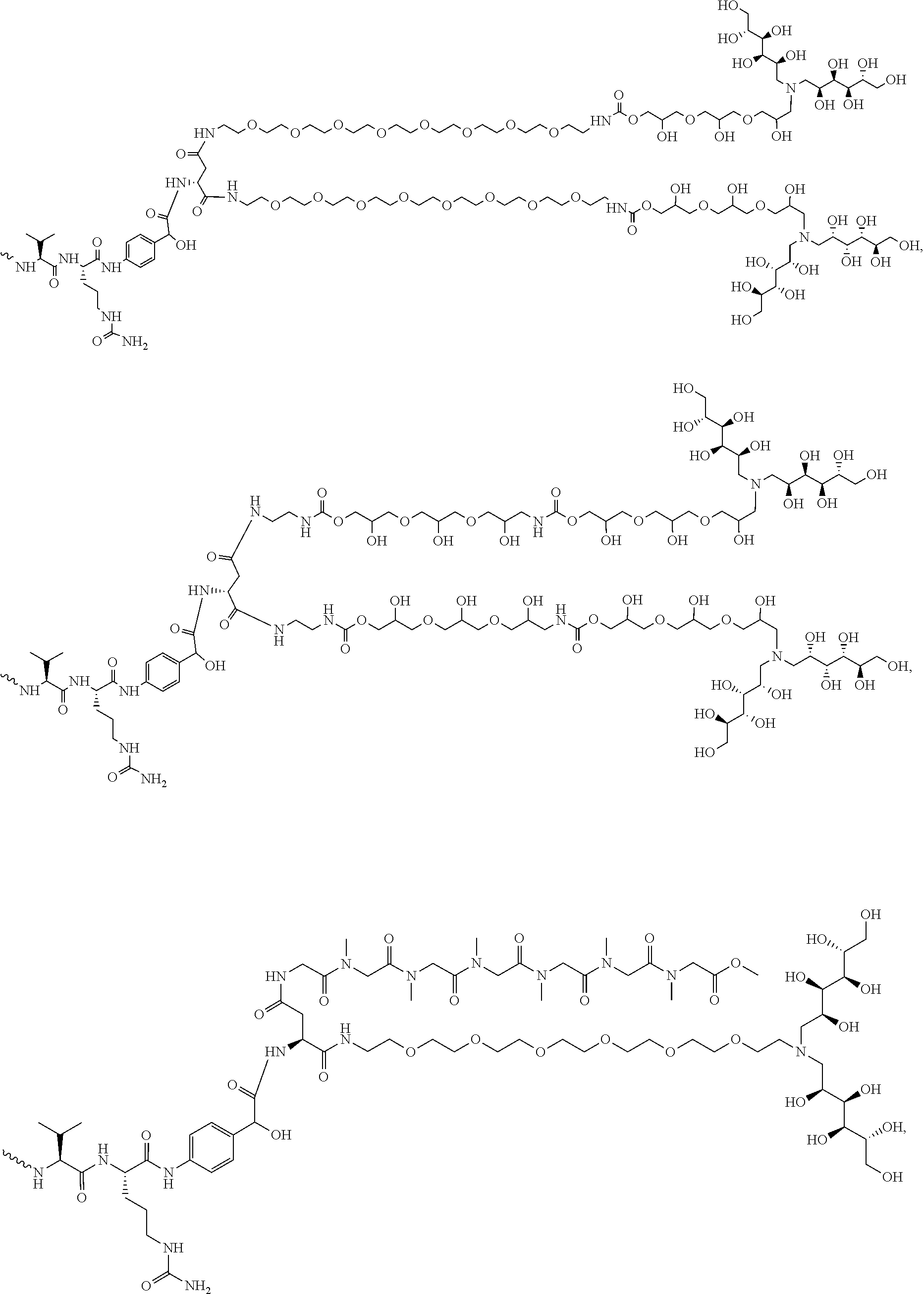

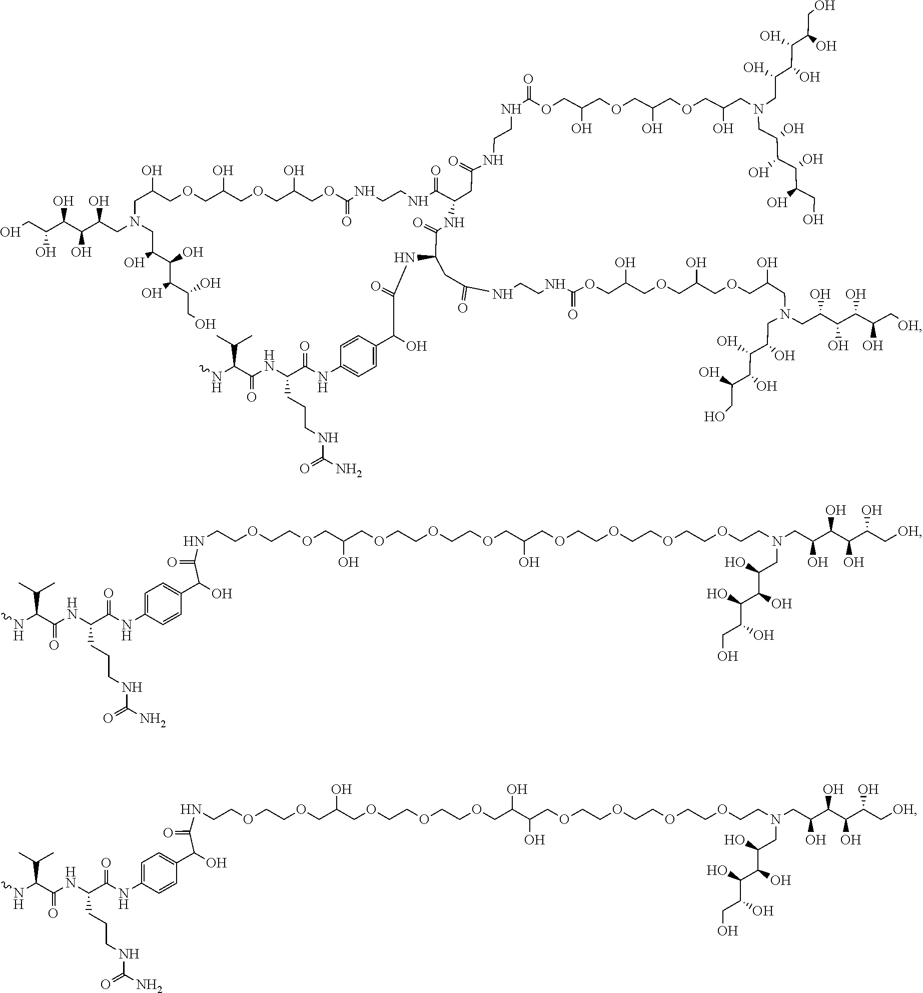

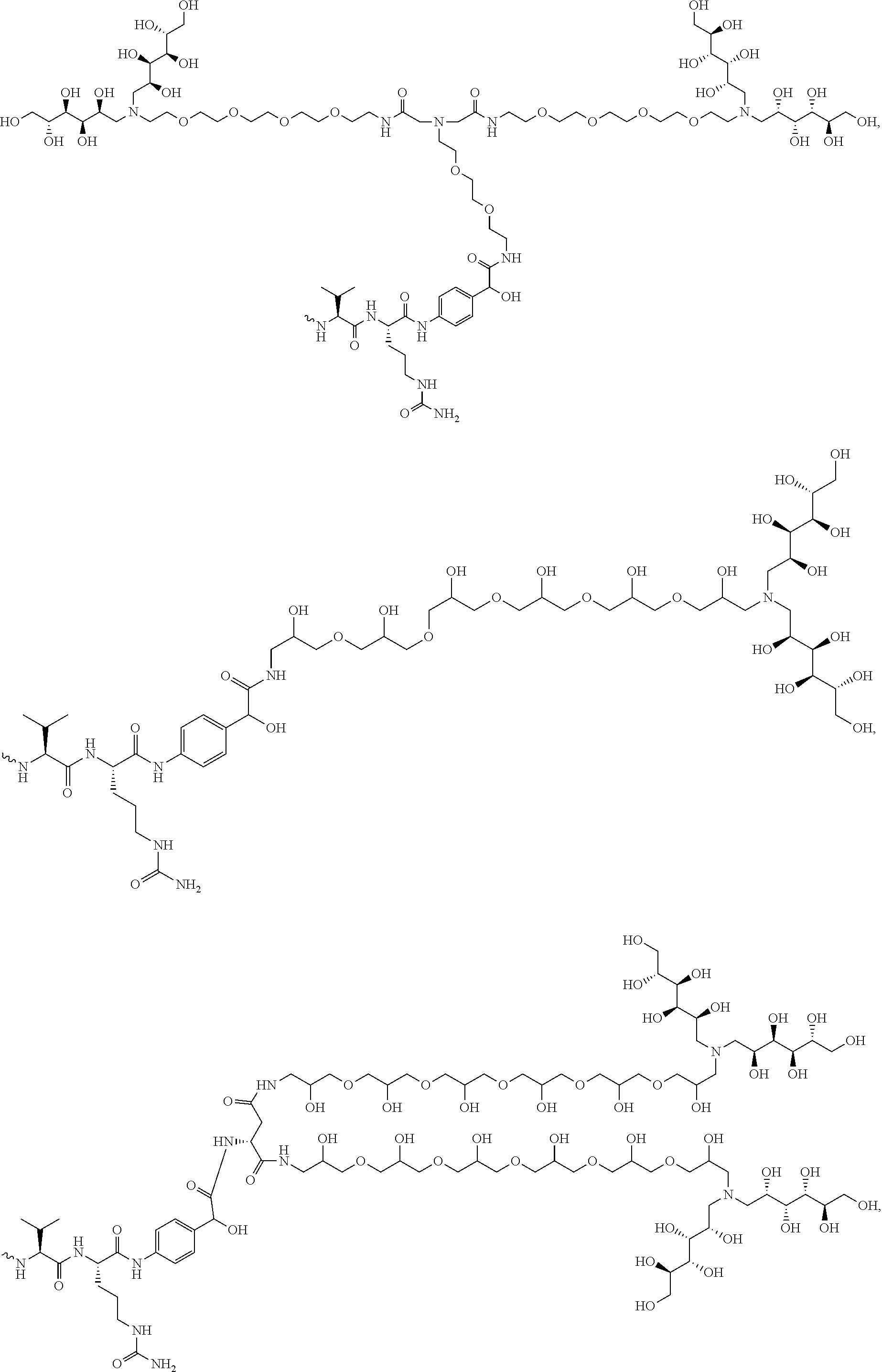

wherein the wavy line on the amino group indicates an attachment site to the stretcher group; or, prior to attachment to the stretcher group, indicates H, and the H on the benzylic OH is optionally replaced with a bond to at least one of the drug units or a linking group attached to the at least one of the drug units.

Embodiment 39. The linker compound of any of Embodiments 1 to 36, wherein the enzyme-cleavable group is joined to the Stretch group by a non-peptidic linking group.

Embodiment 40. The linker compound of Embodiment 39, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

Embodiment 41. The linker compound of any of Embodiments 1-40, comprising the stretcher group attached to the enzyme-cleavable group.

Embodiment 42. The linker compound of Embodiment 41, wherein the stretcher group is selected from the following:

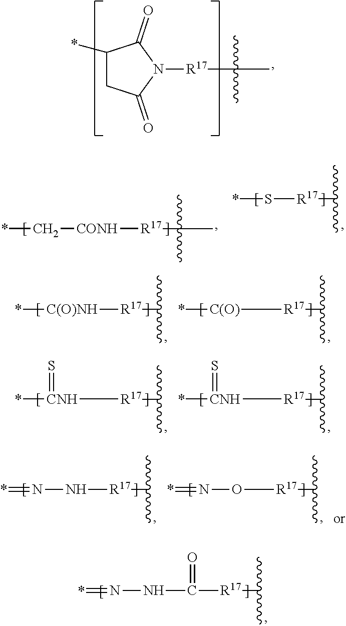

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)b-C₁-C₈ alkylene- (where b is 1 to 26), —C₁-C₈ alkylene-(CH₂—O—CH₂)b- (where b is 1 to 26), —C₁—C alkylene-(CH₂—O—CH₂)b-C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃—C₈ carbocyclo)-, —(C₃-C₈ carbocyclo)-C₁-C₀₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the stretcher group comprises maleimido(C₁-C₁₀ alkylene-C(O)—, maleimido(CH₂OCH₂)ₚ₂(C₁-C₁₀alkylene)C(O)—, maleimido(C₁-C₁₀ alkylene) (CH₂OCH₂)ₚ₂C(O)—, or a ring open form thereof, wherein p2 is from 1 to 26;
and wherein * is an attachment to the PTK7 binding agent, and the wavy line is an attachment to the enzyme-cleavable group.

Embodiment 43. The linker compound of Embodiment 41, wherein the stretcher group is selected from the following:

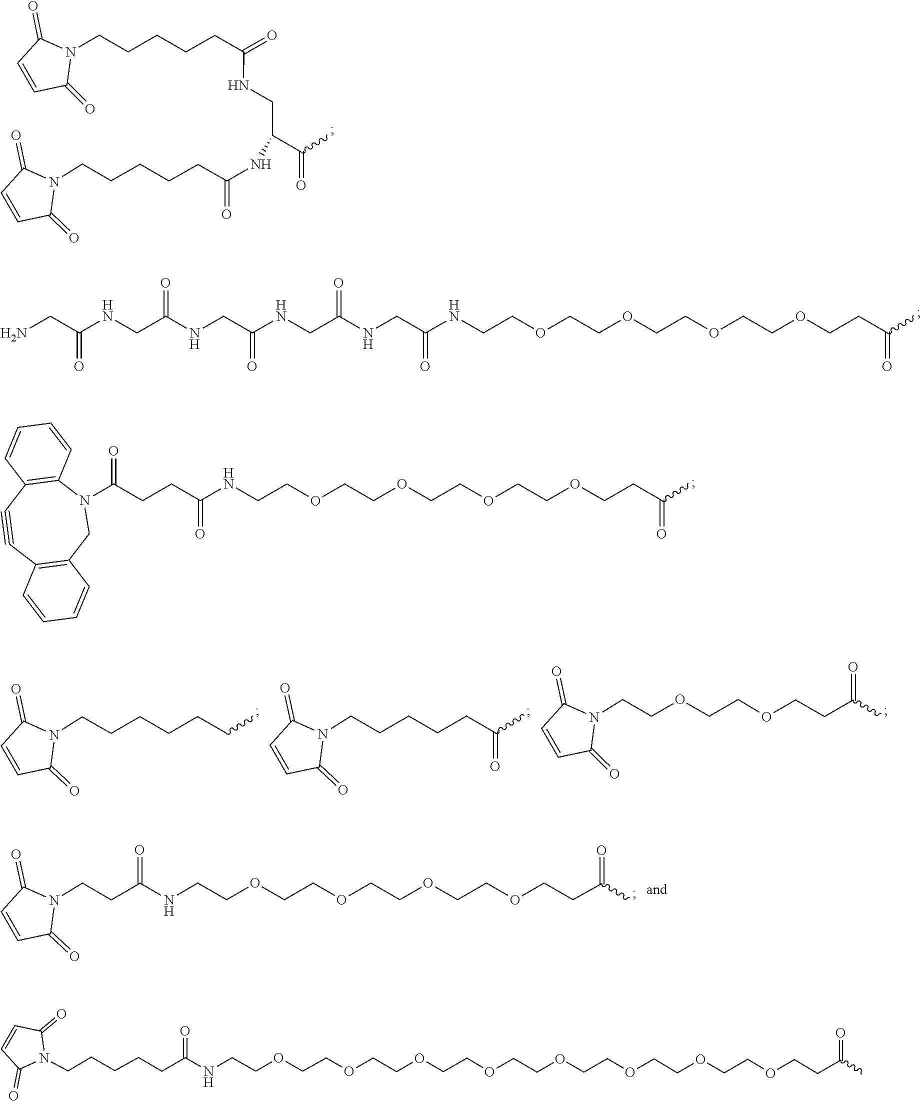

wherein the wavy line [symbol] indicates an attachment site of the stretcher group to the enzyme-cleavable group, and the attachment site to the PTK7 binding agent is on the maleimide, primary amine or alkyne functional group.

Embodiment 44. The linker compound of Embodiment 1, having one of the following structures:

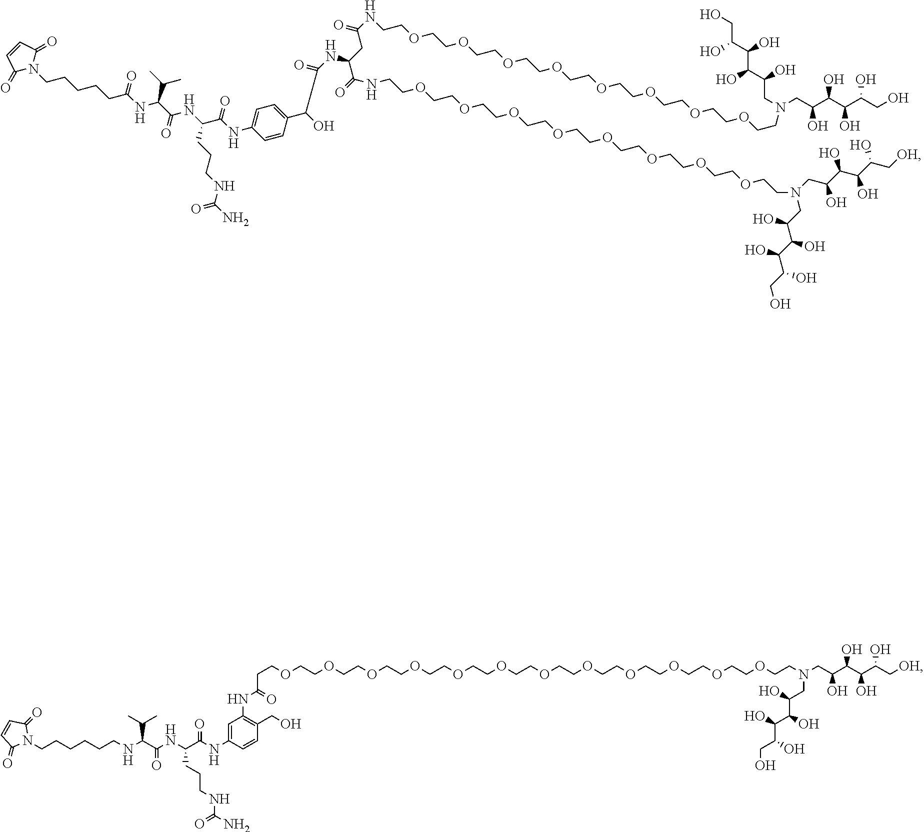

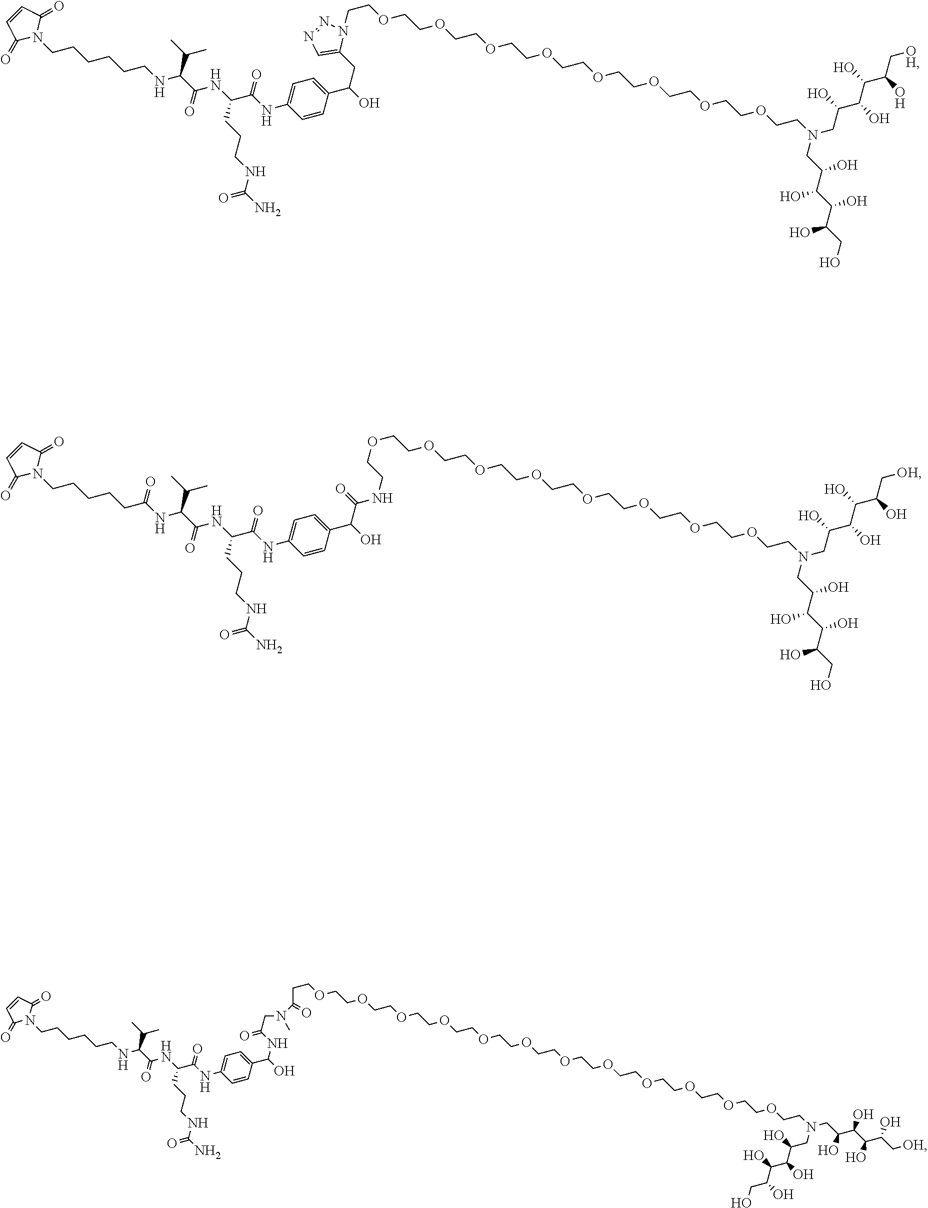

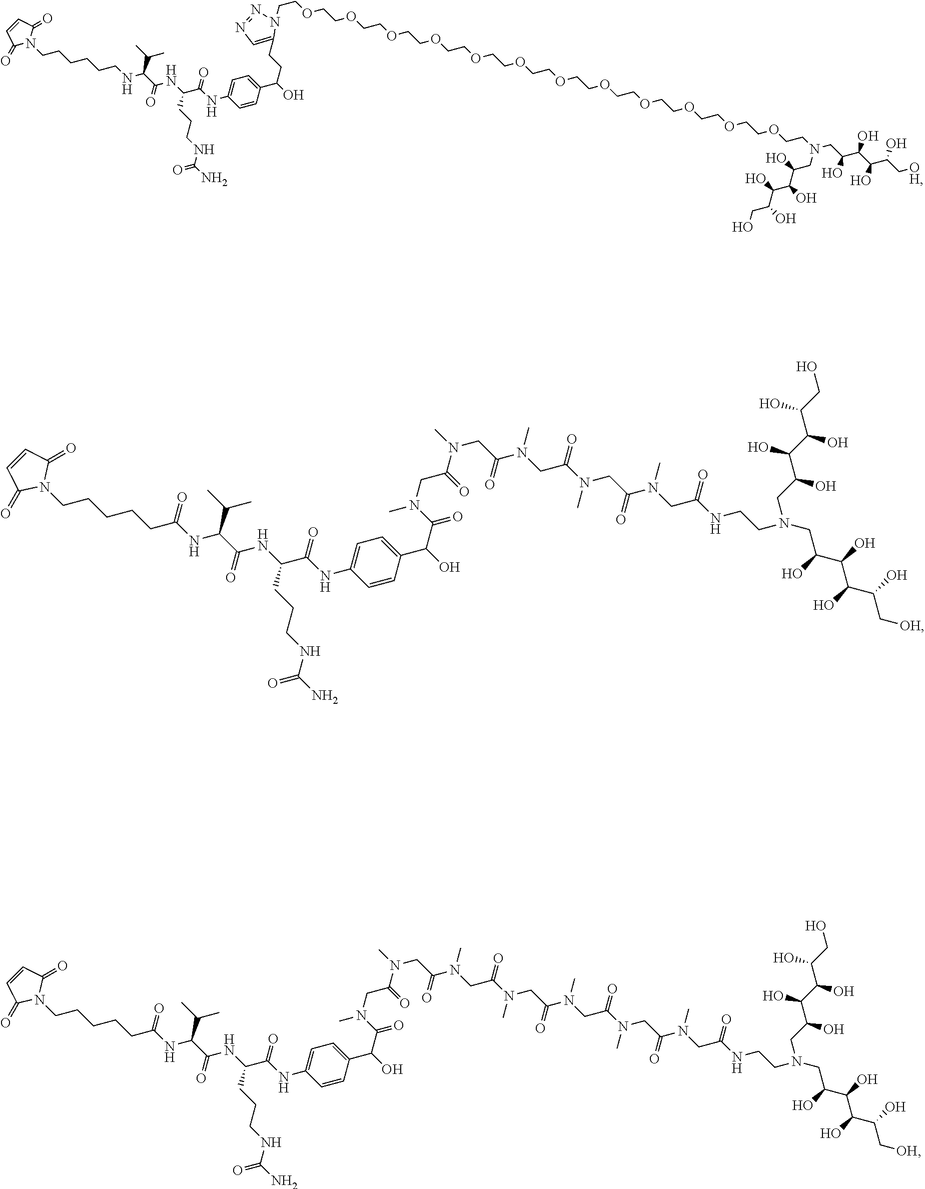

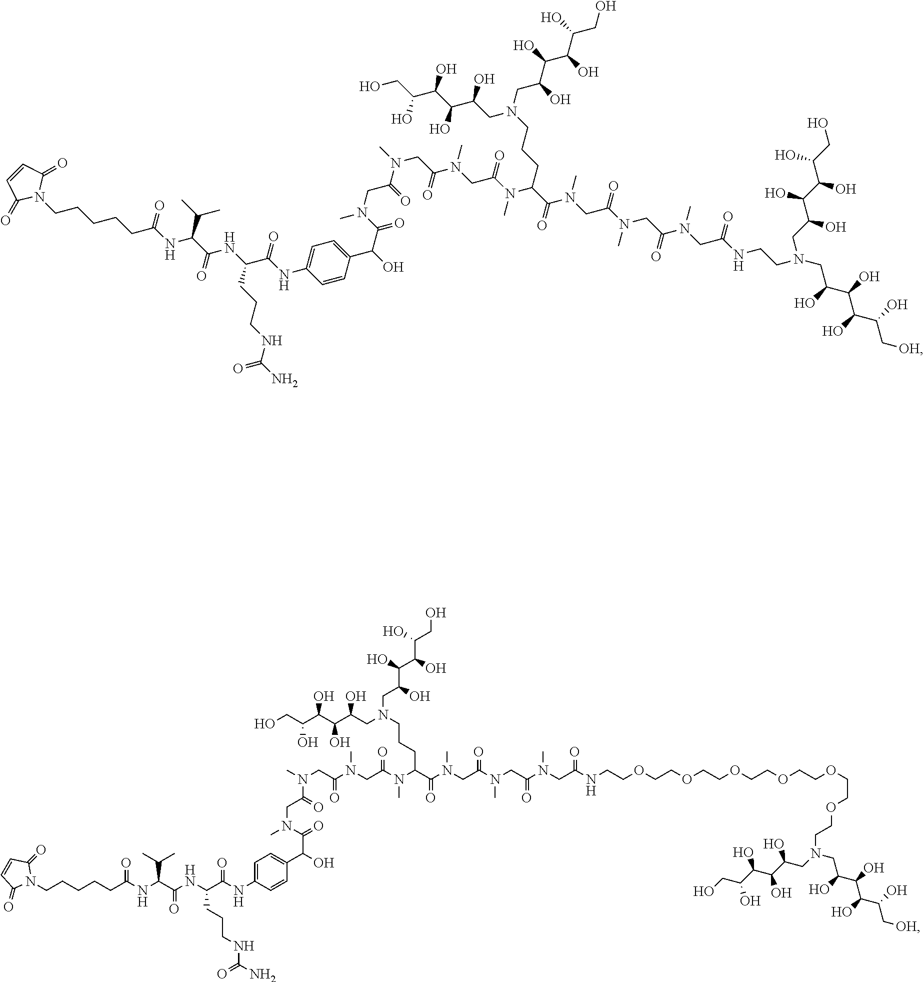

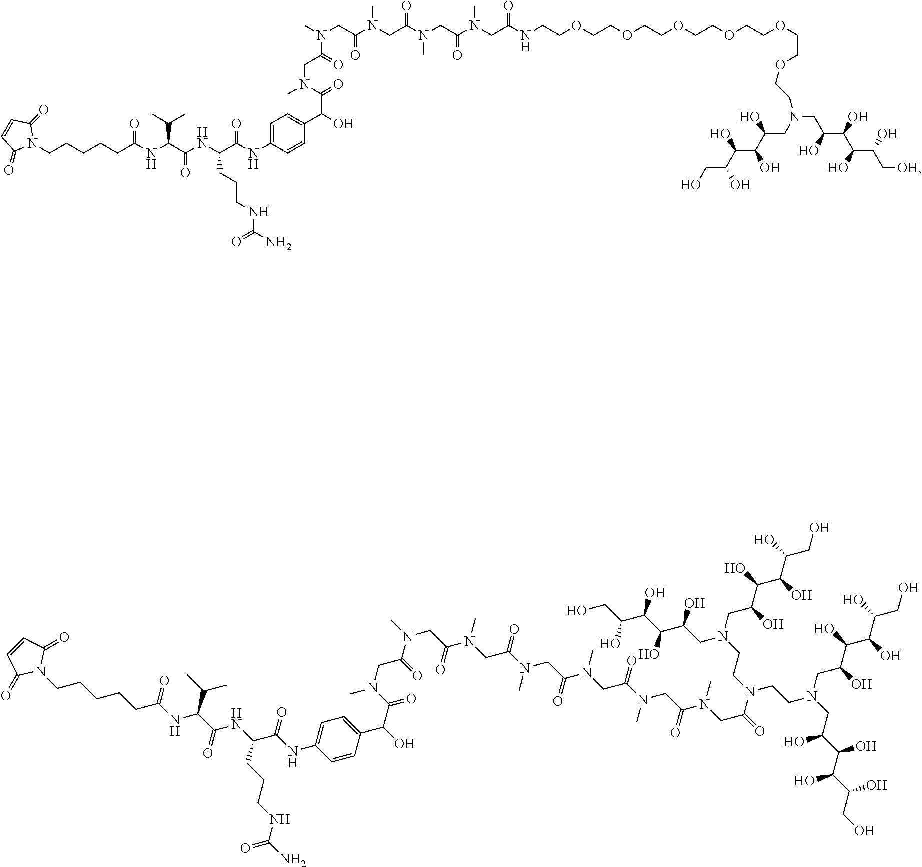

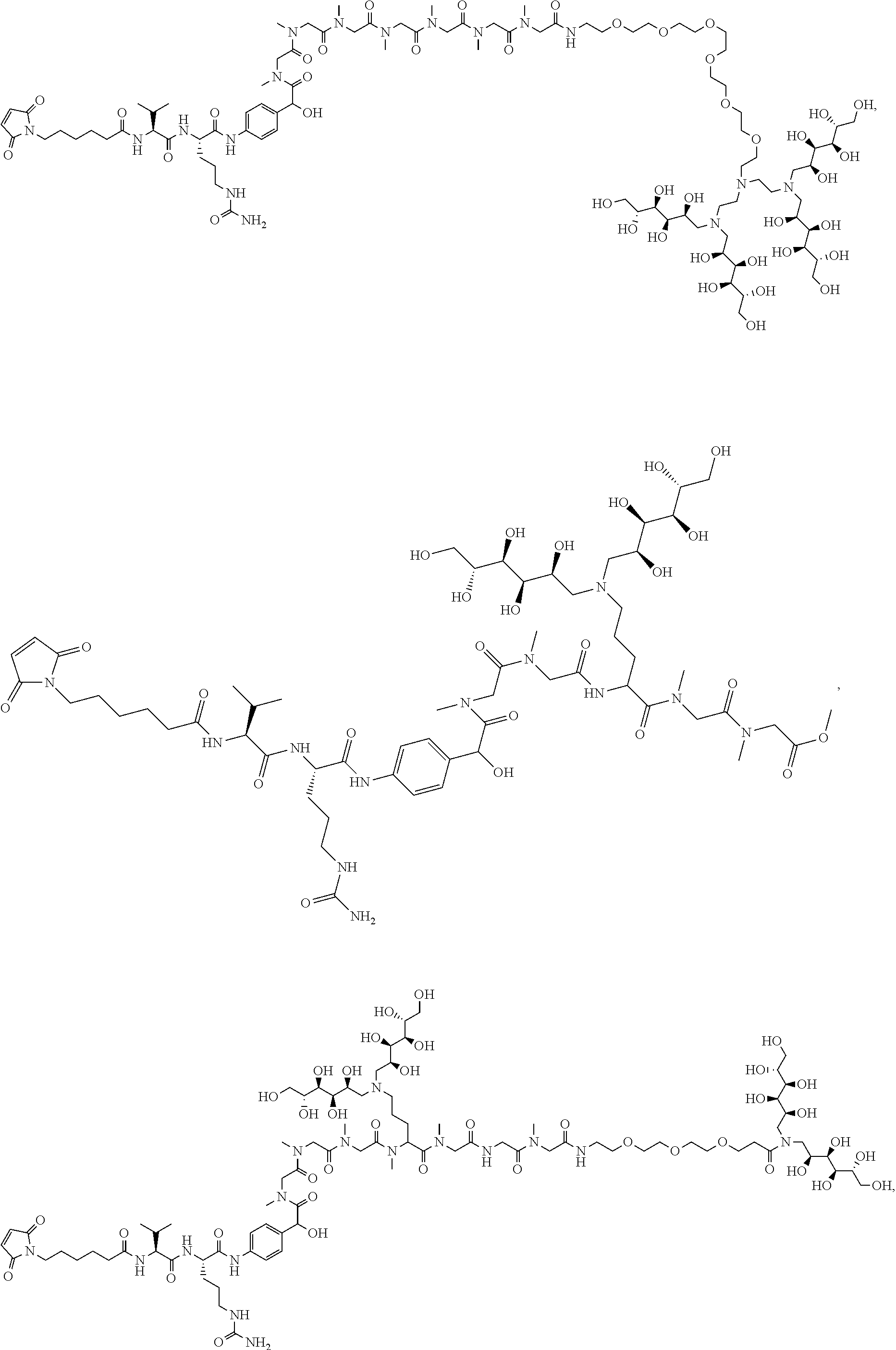

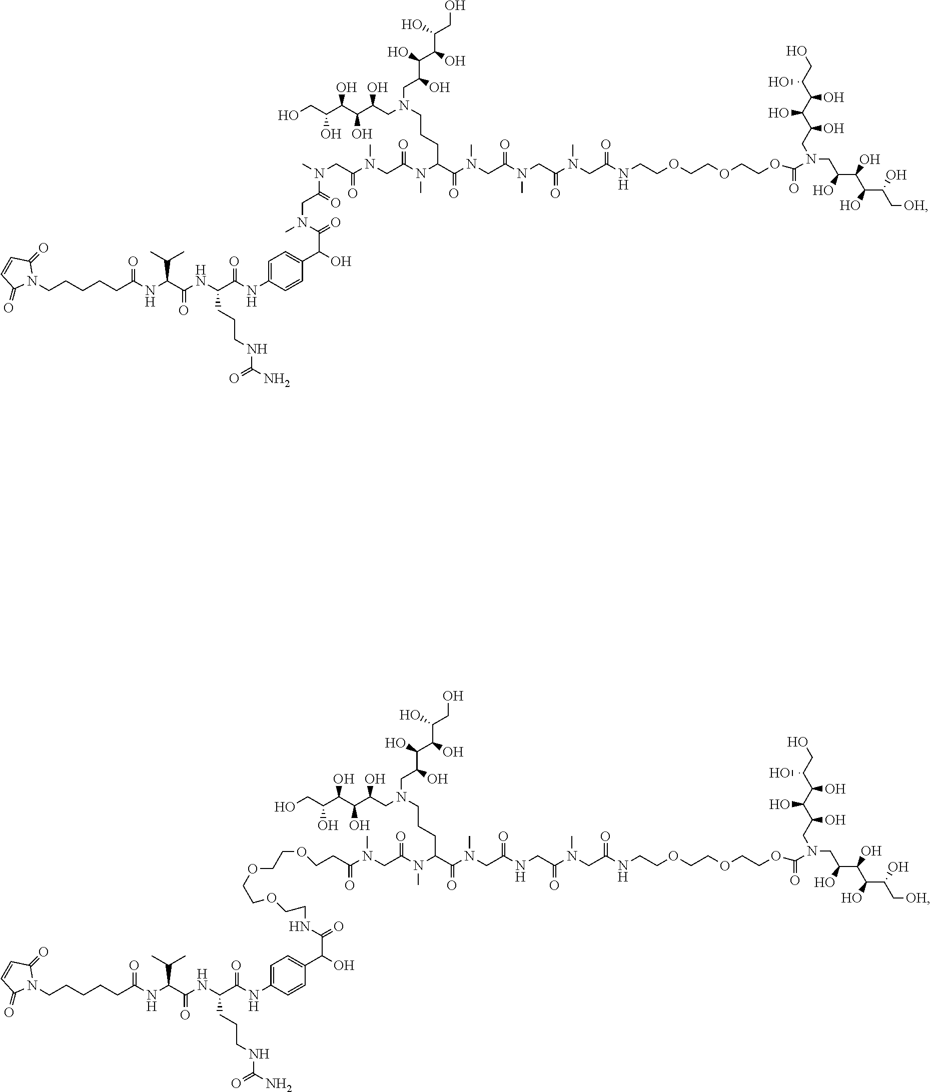

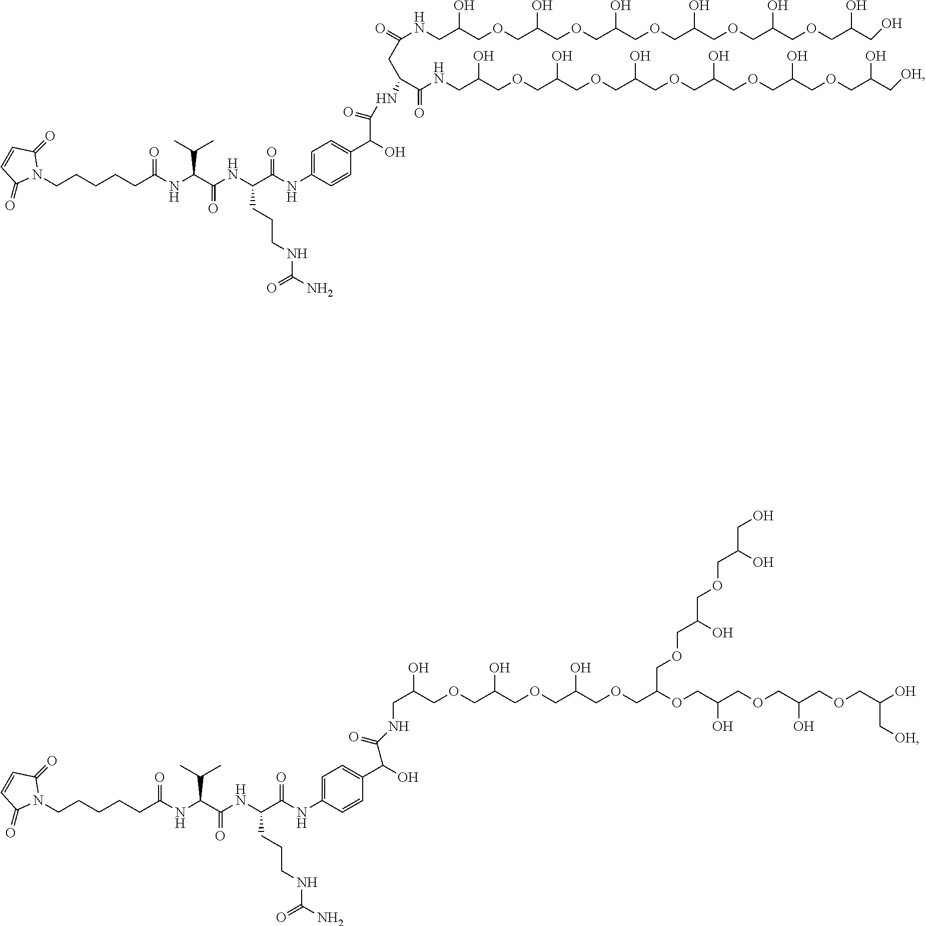 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(COCC(O)COCC(O)CO)OCC(O)COCC(O)COCC(O)CO)cc1]

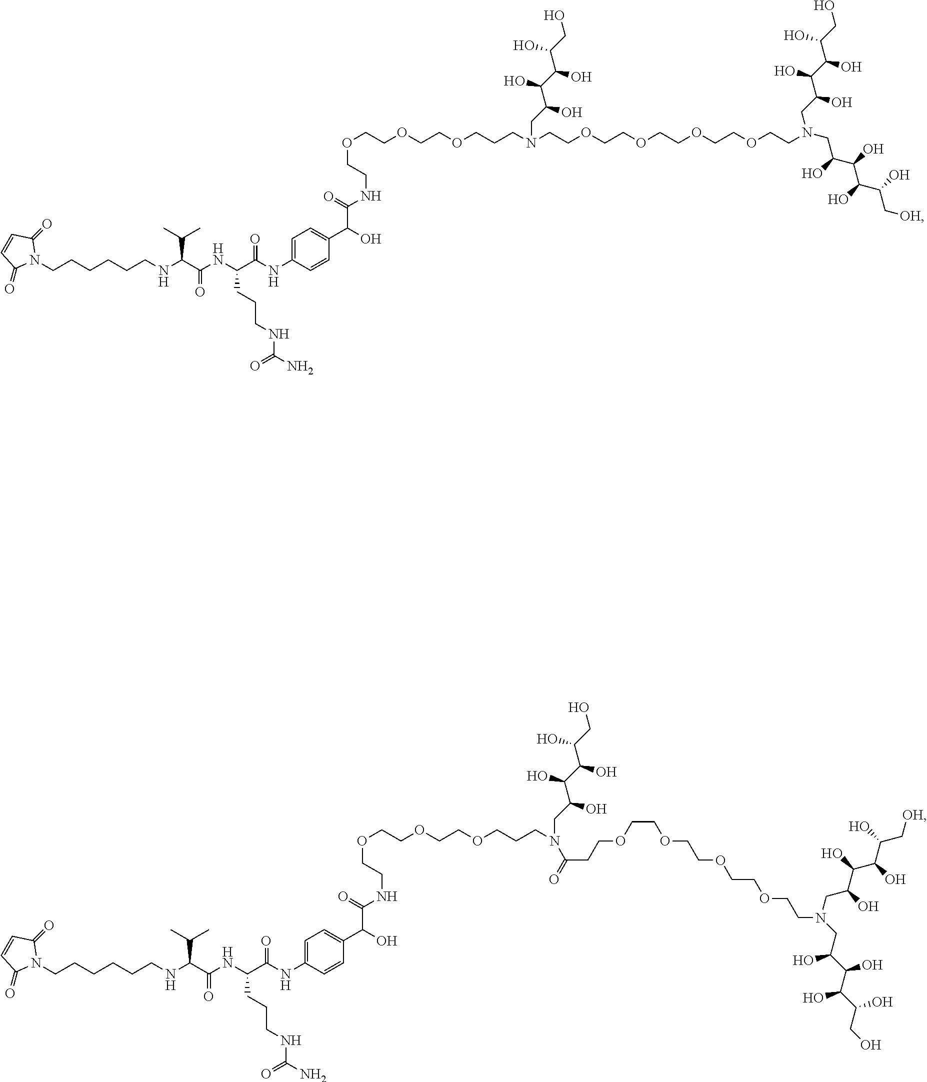 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1 ; CC(C)[C@H](NCCCCCCN1C(=O)C=CC1=O)C(=O)N[C@@H](CCCNC(N)=O)C(=O)Nc1ccc(C(O)C(=O)NCCOCCOCCOCCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)cc1]

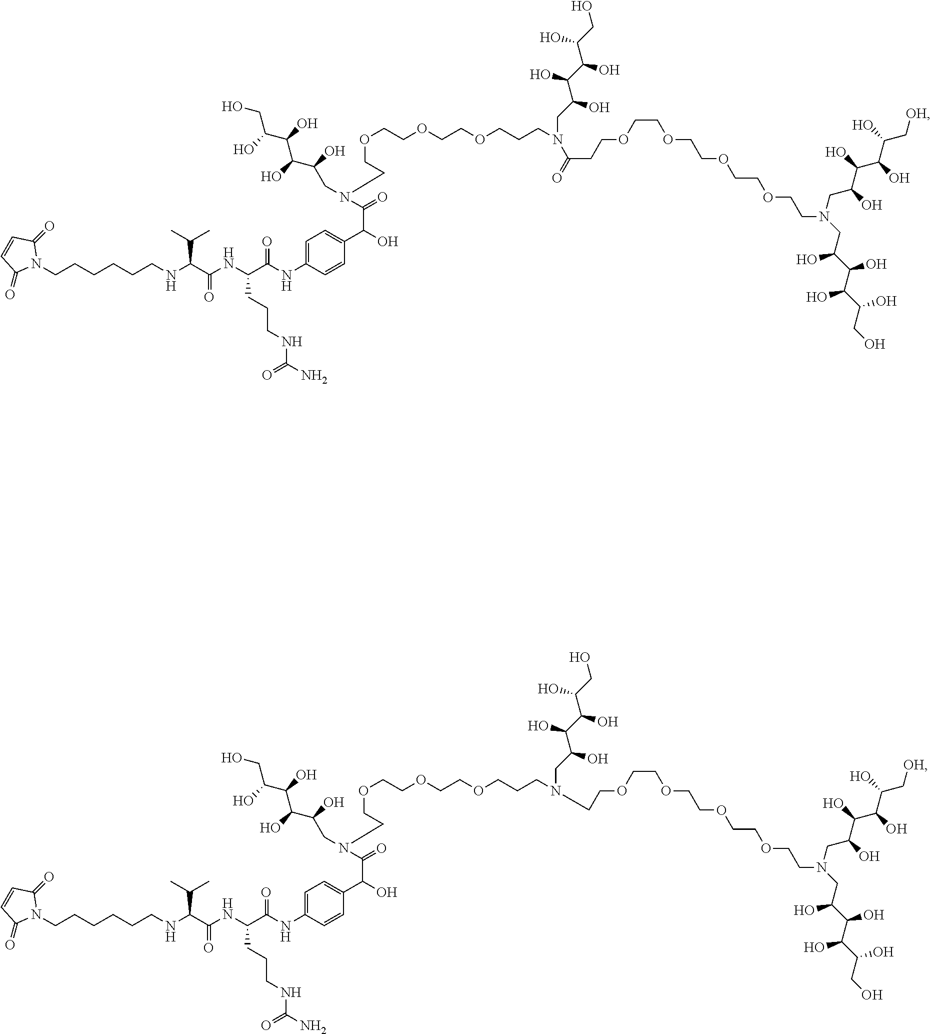

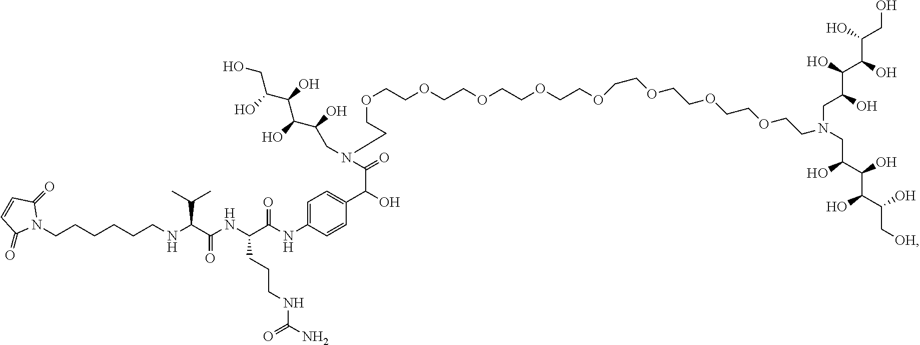 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

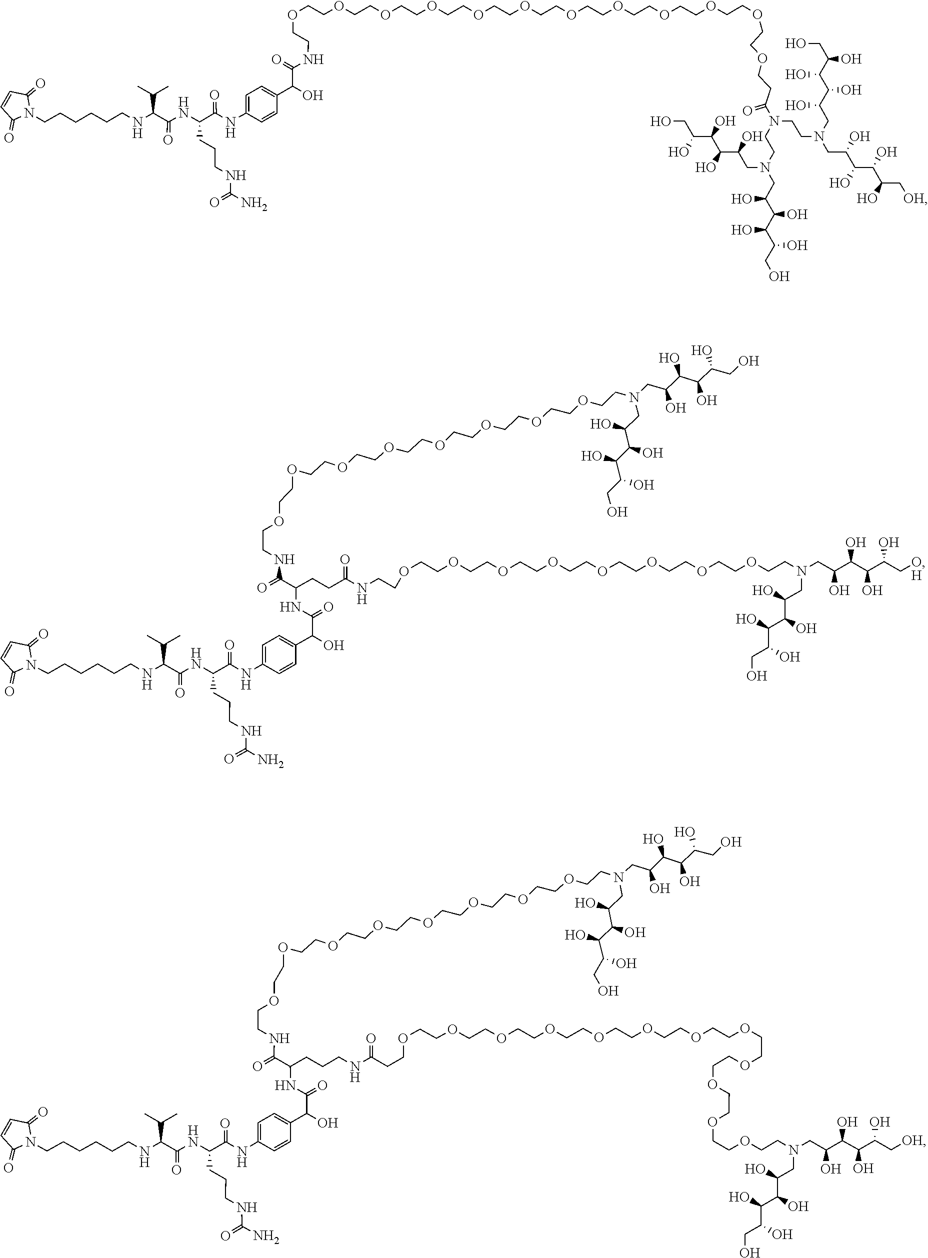

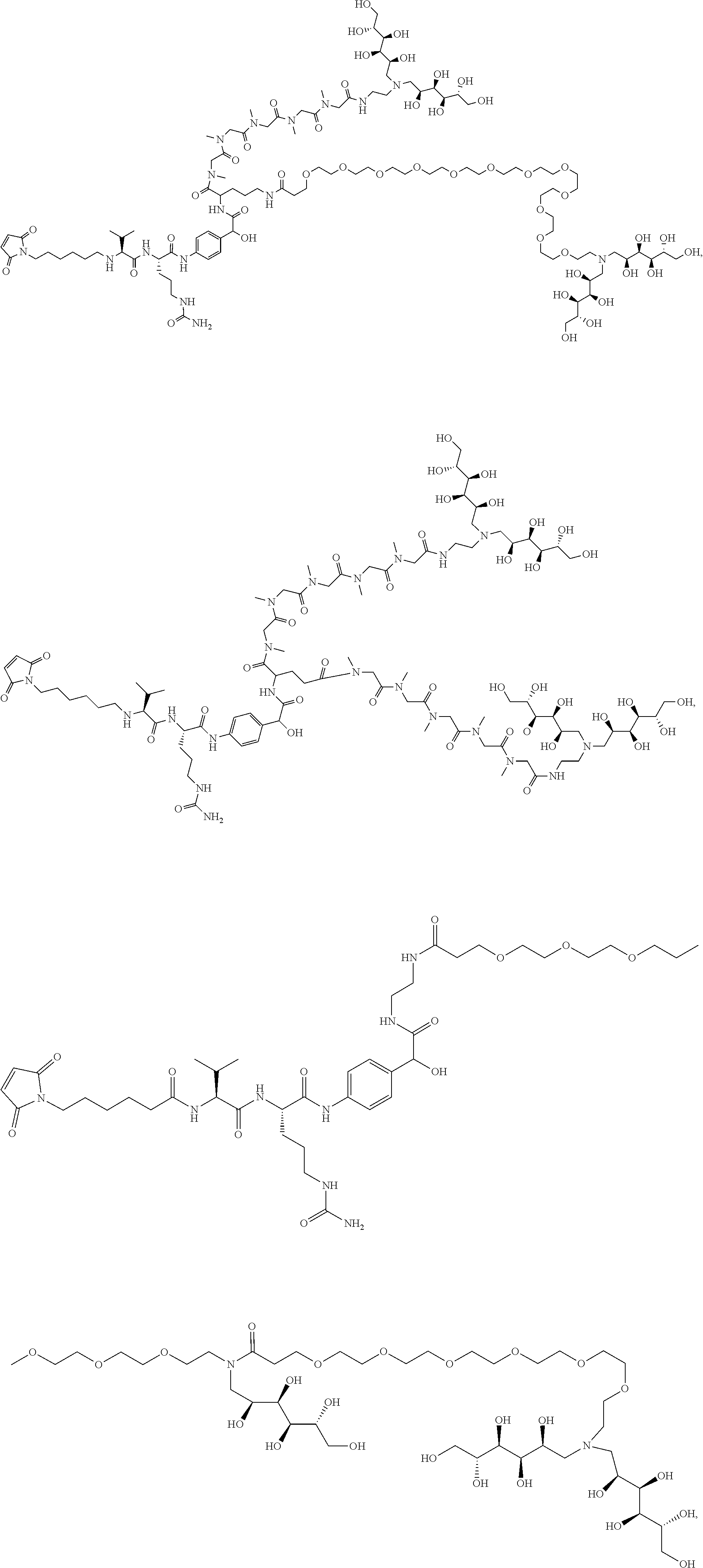

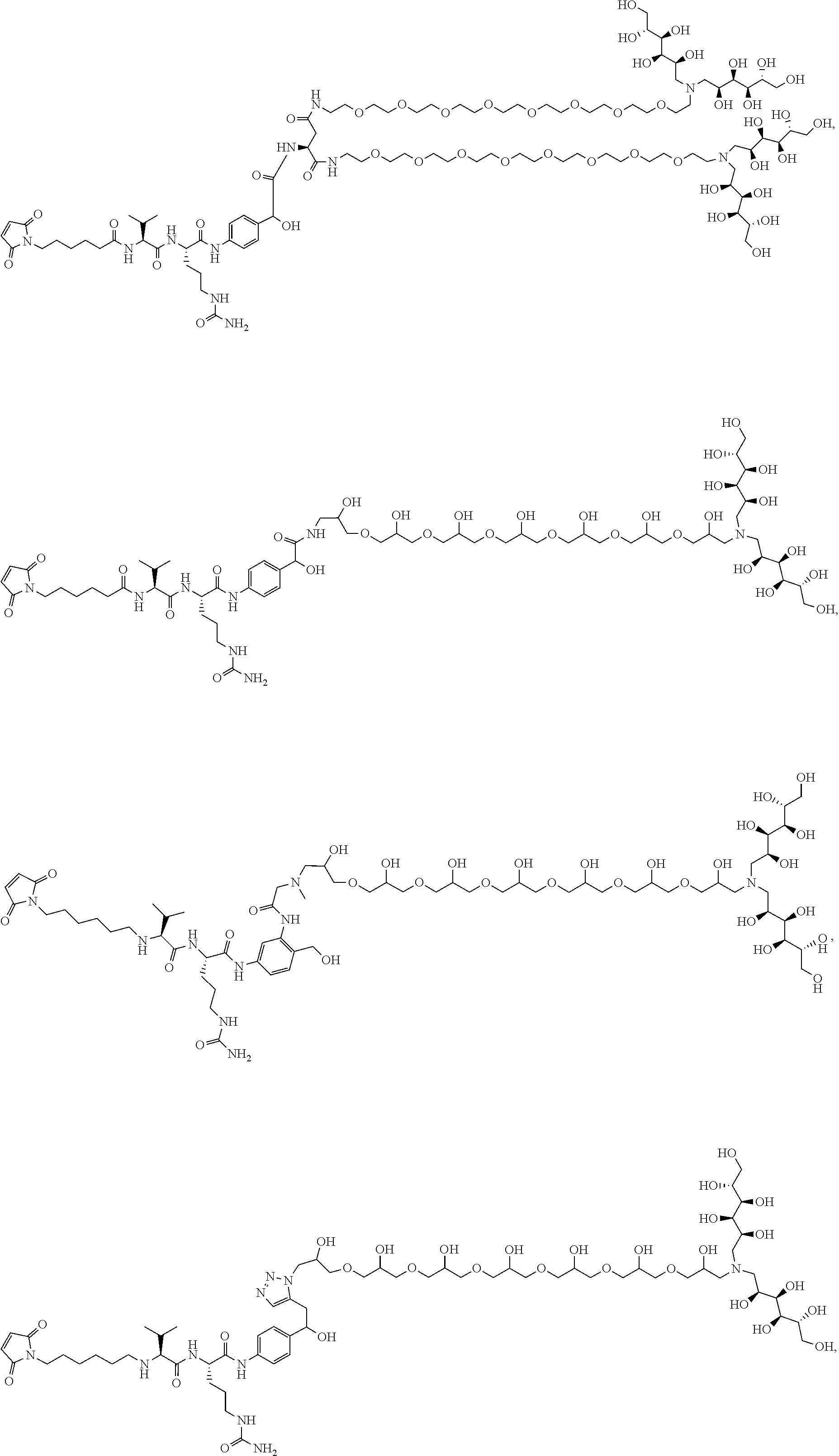

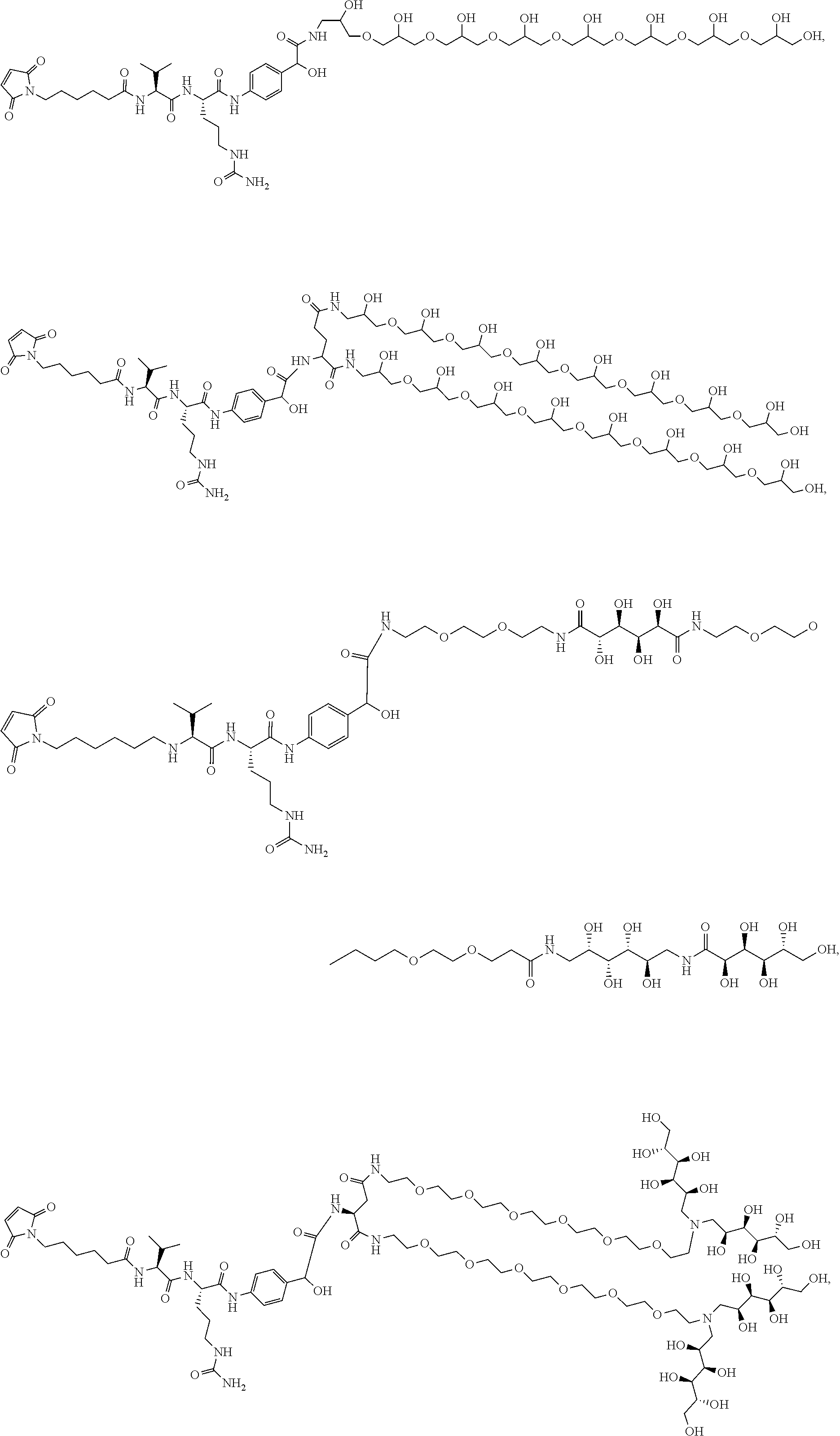

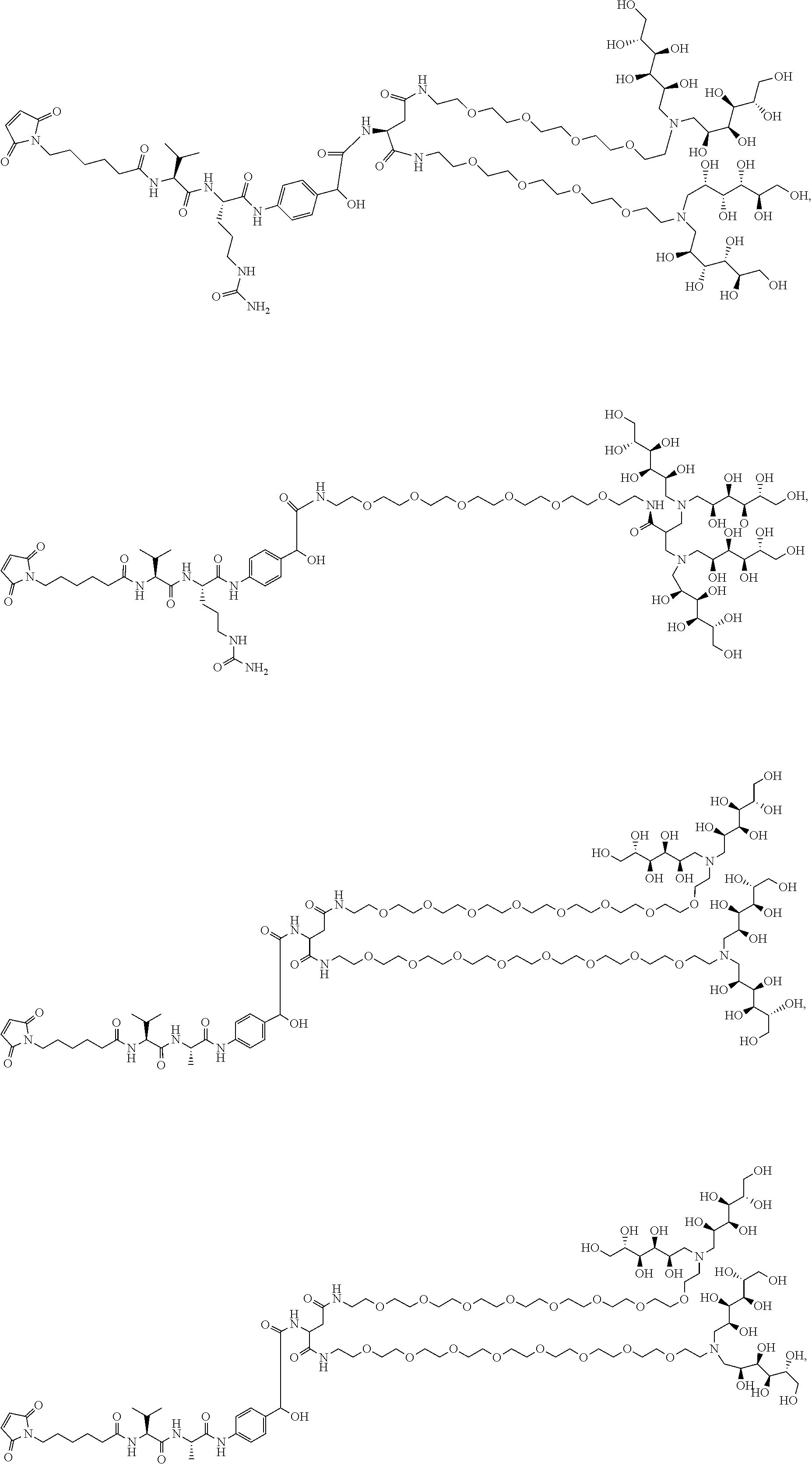

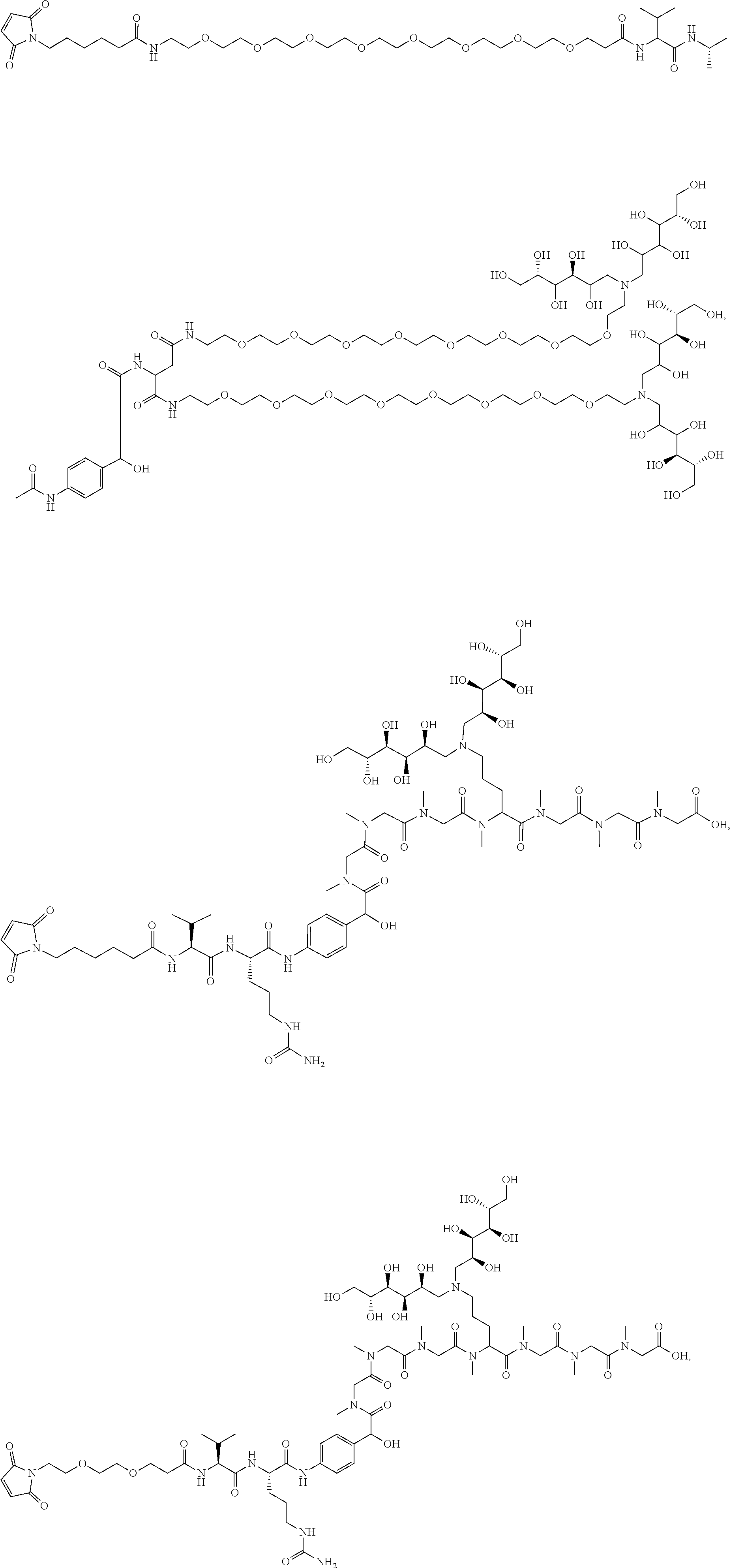

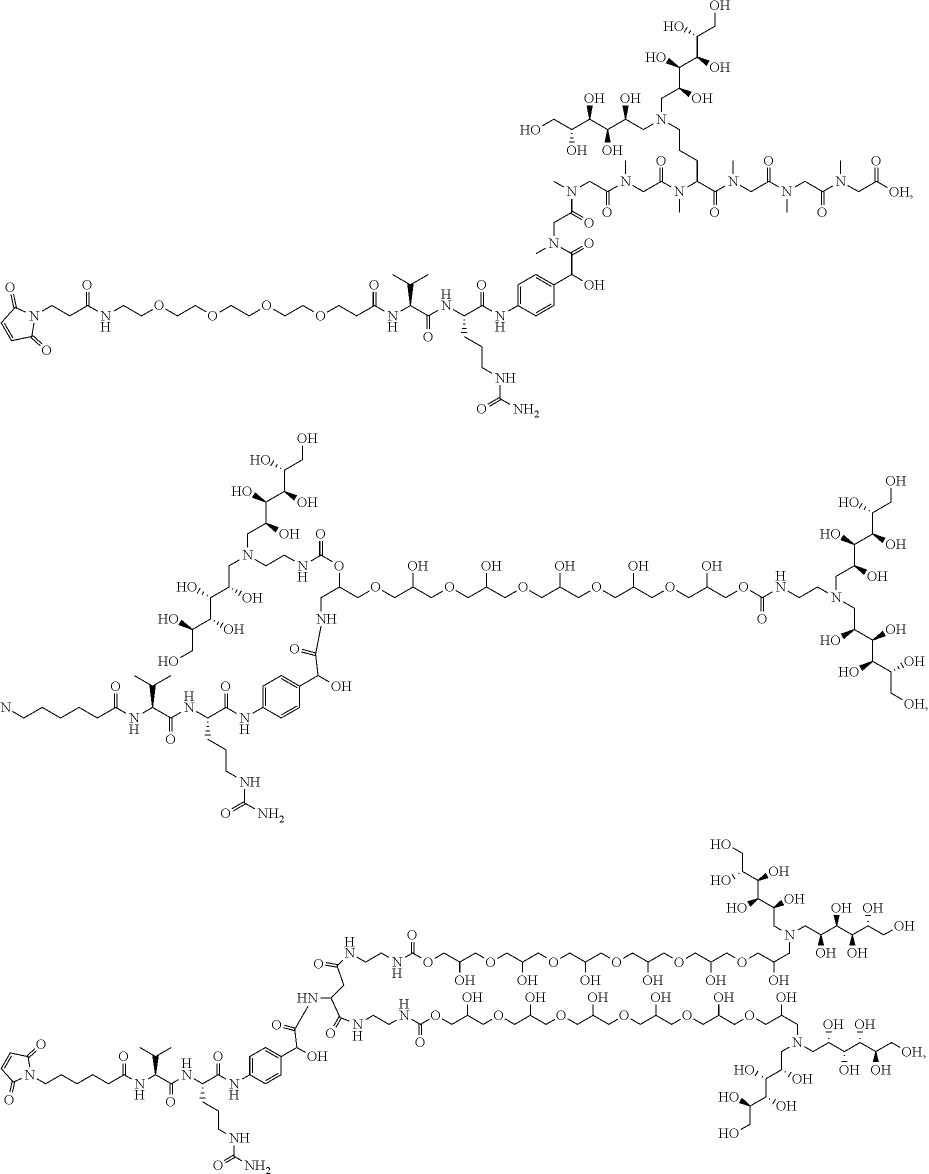

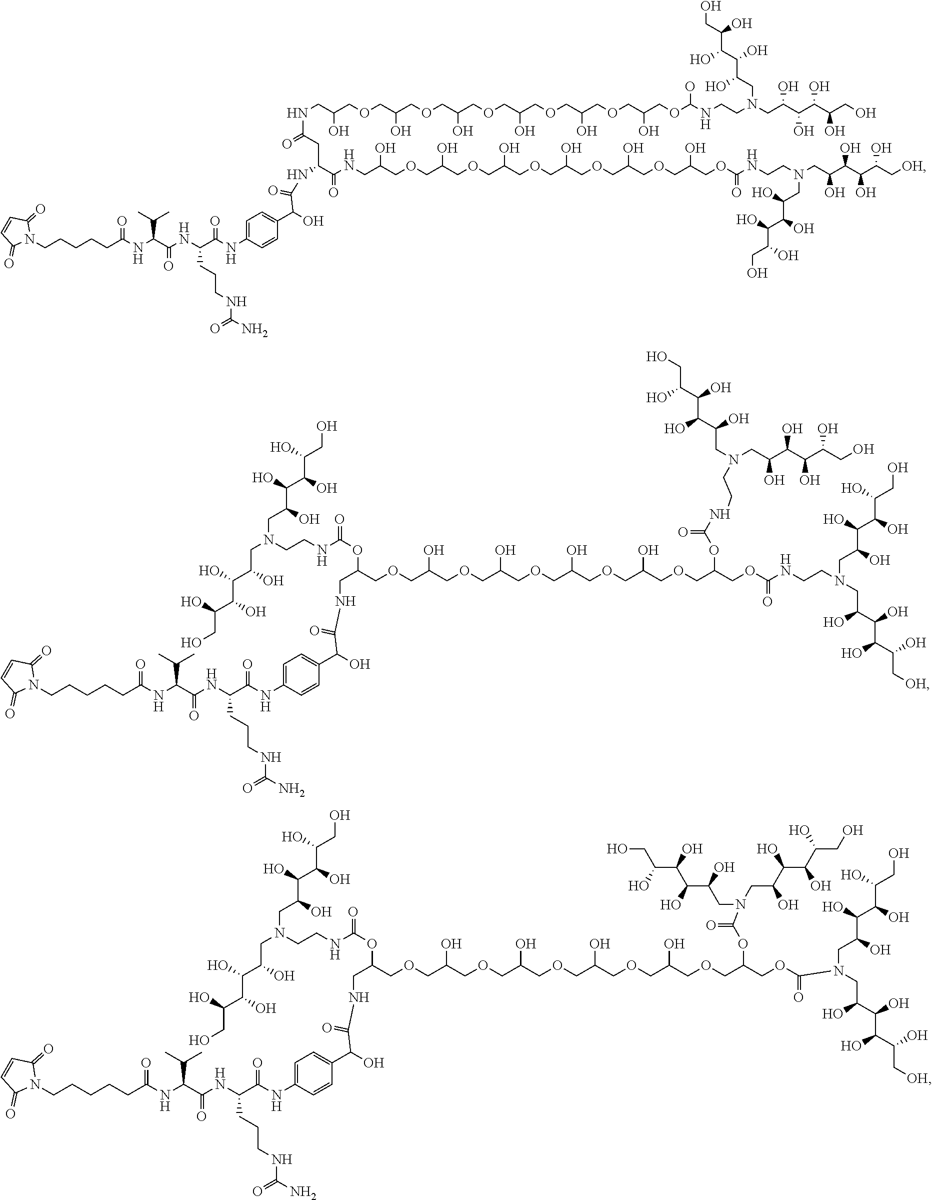

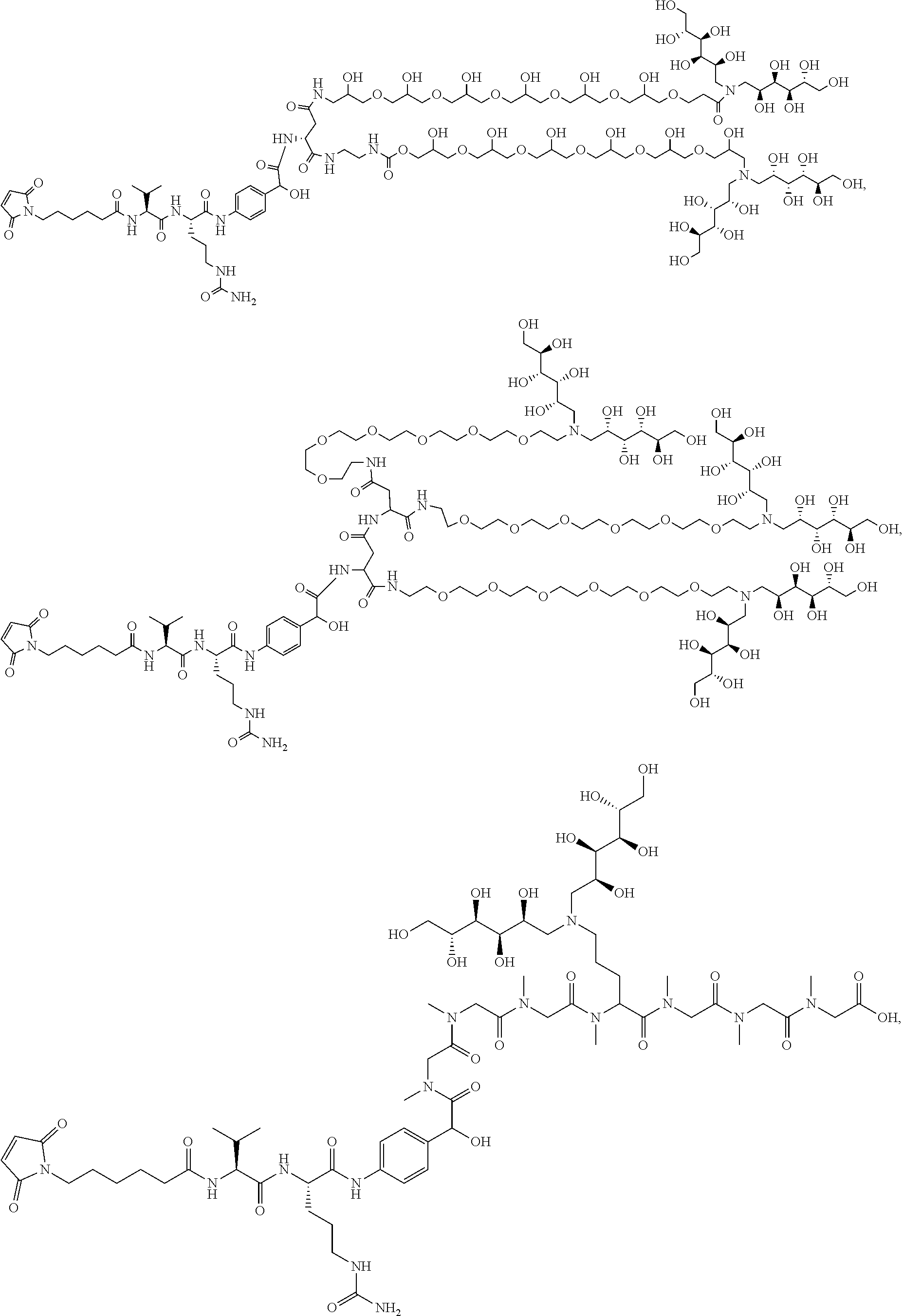

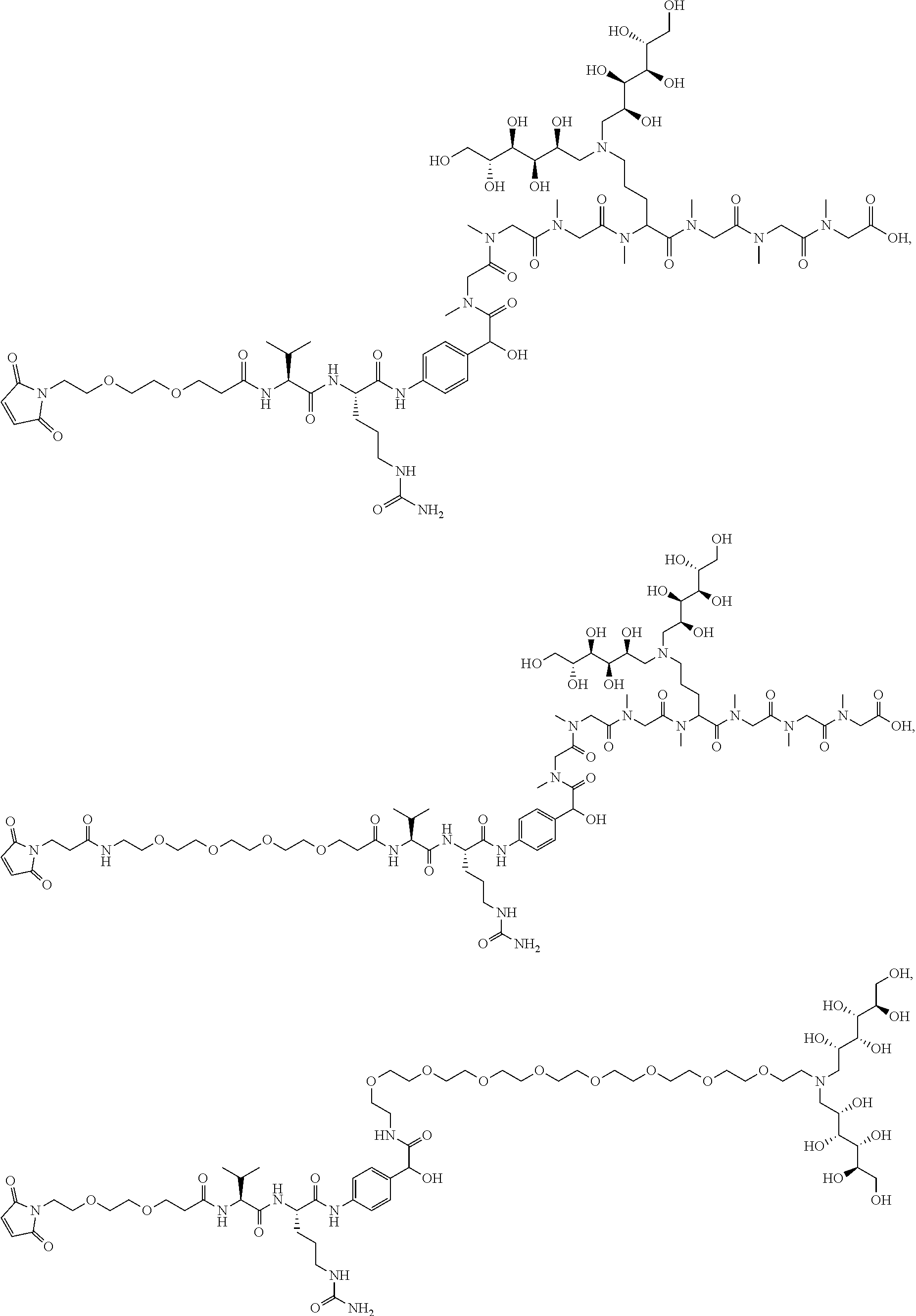

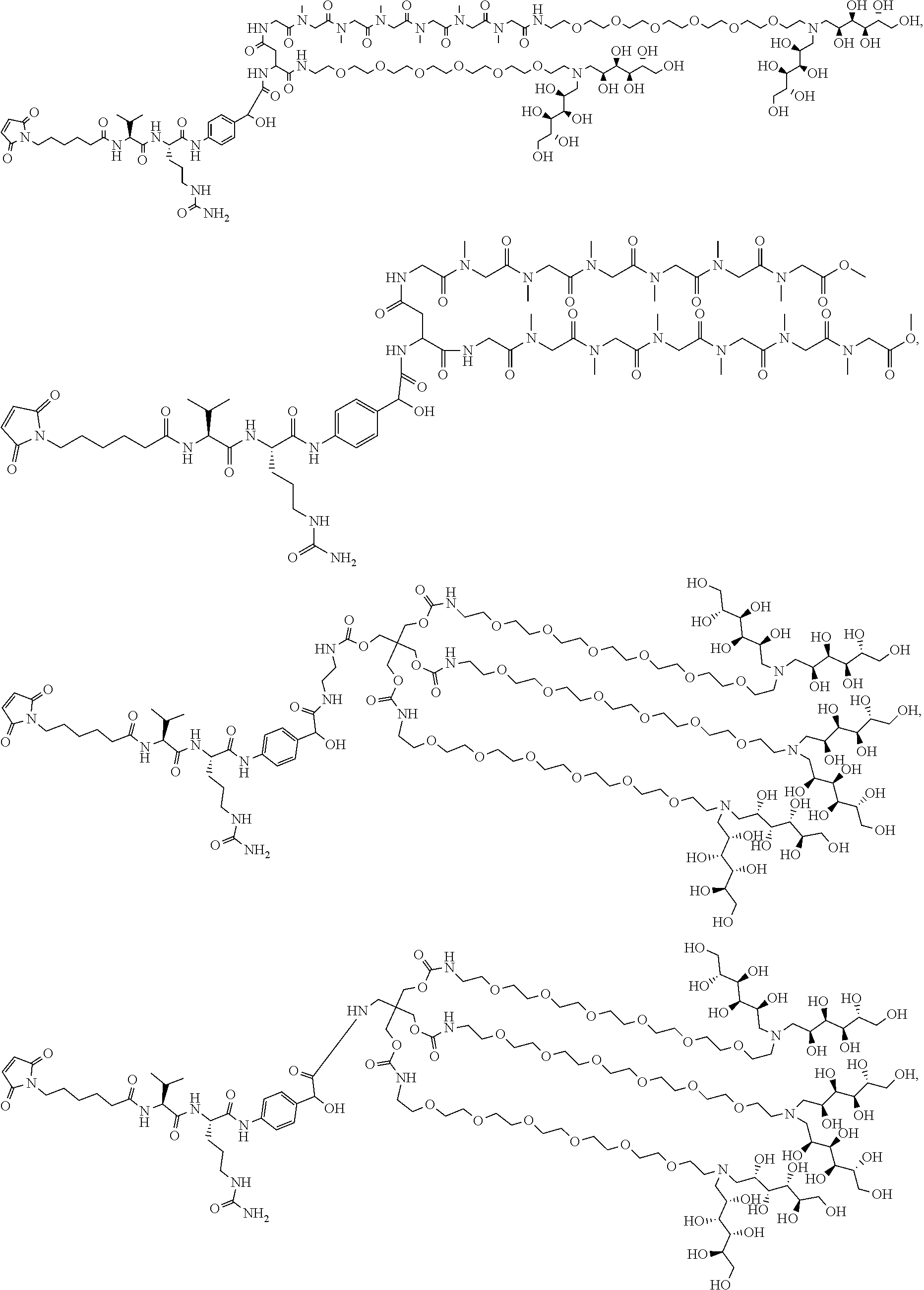

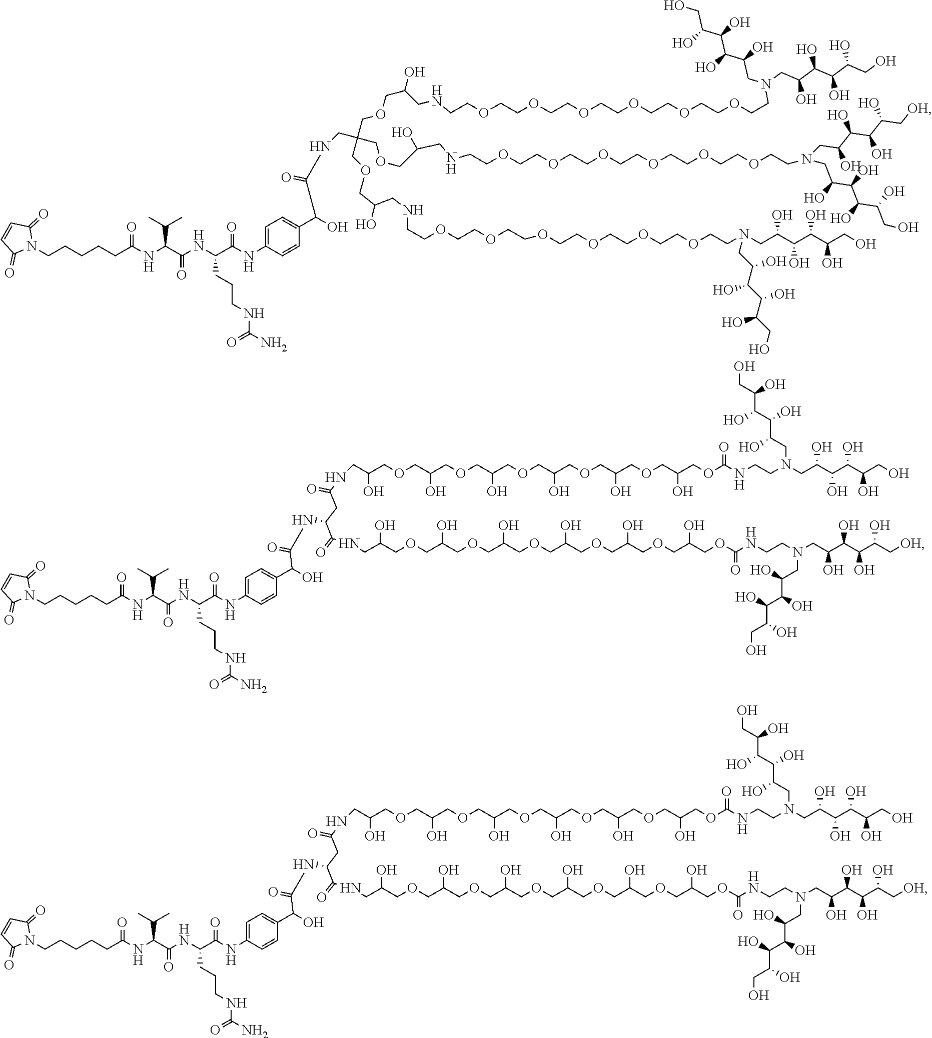

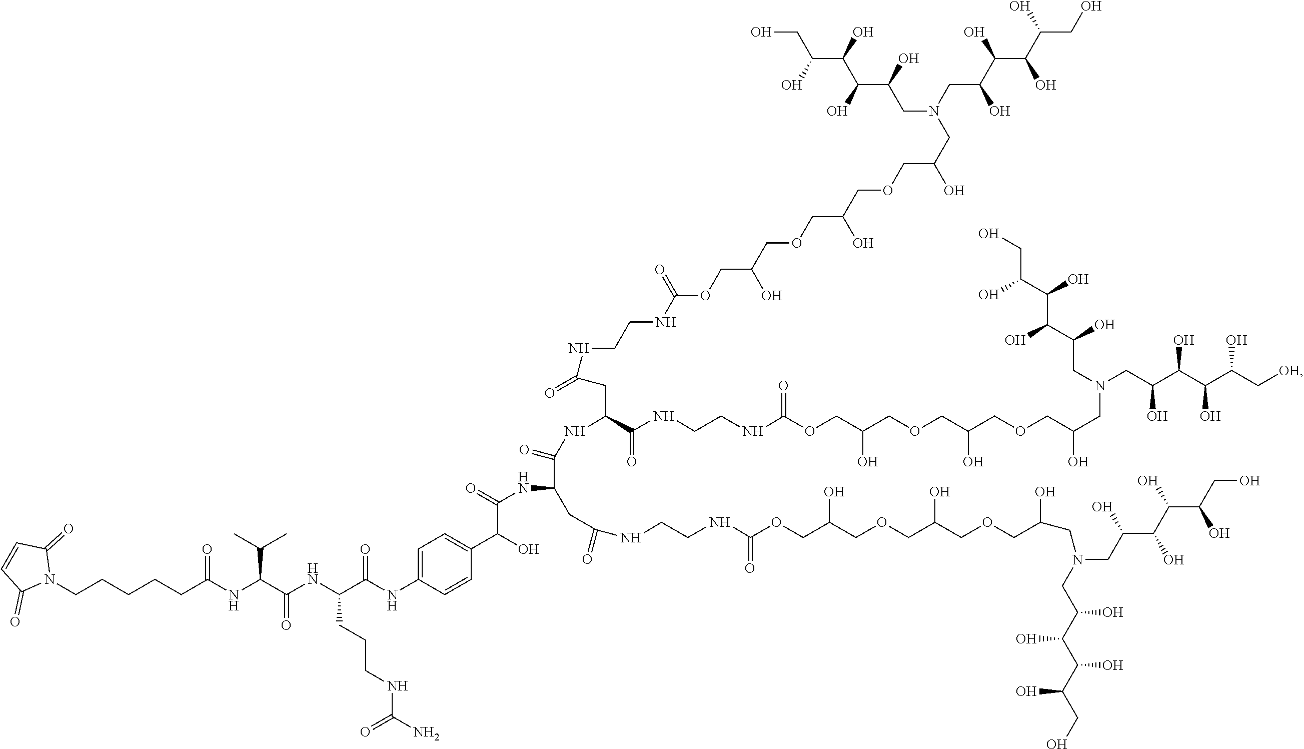 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

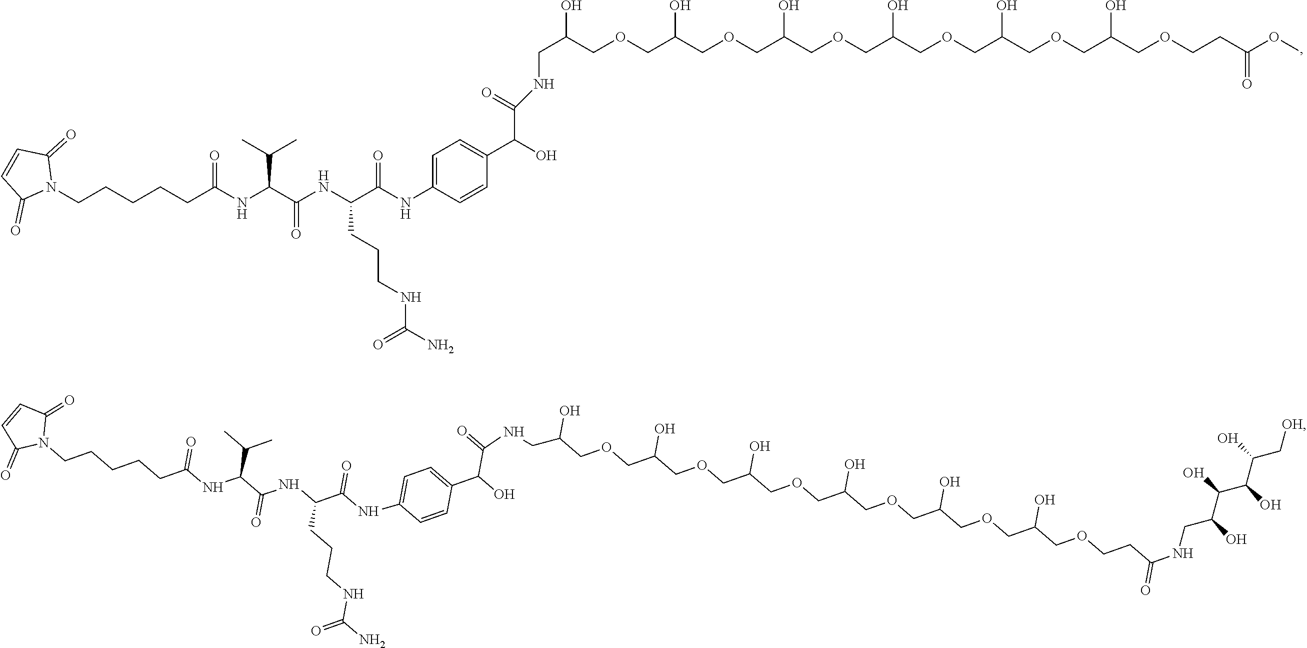

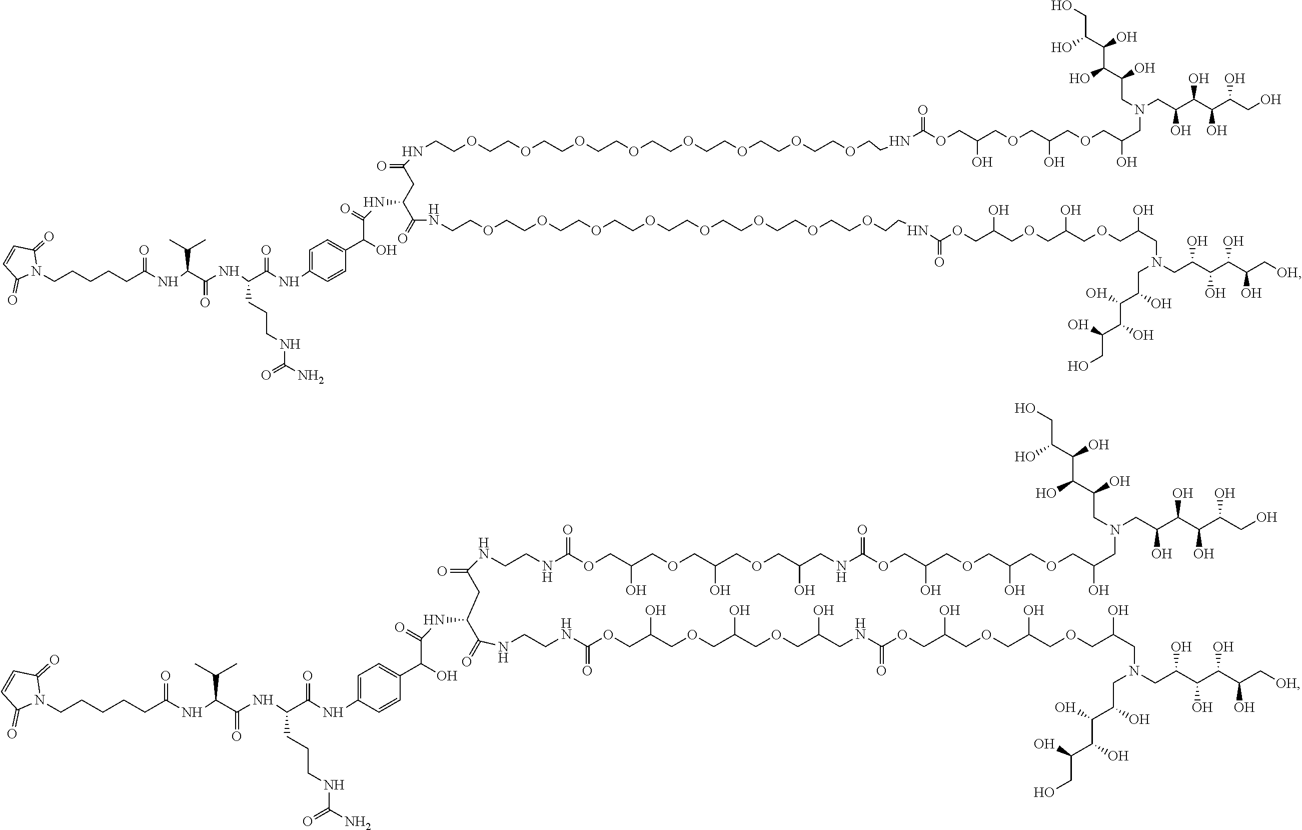

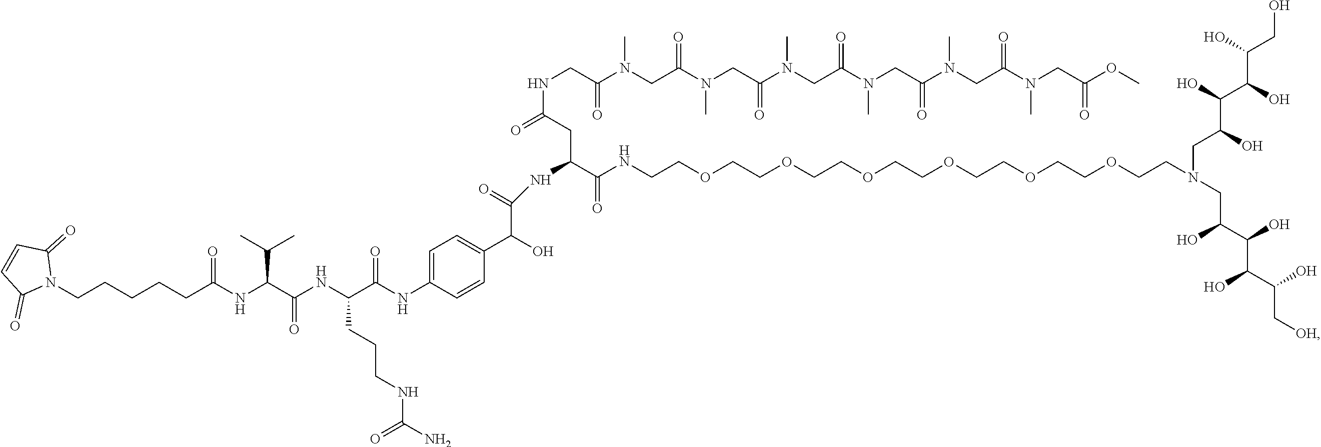 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

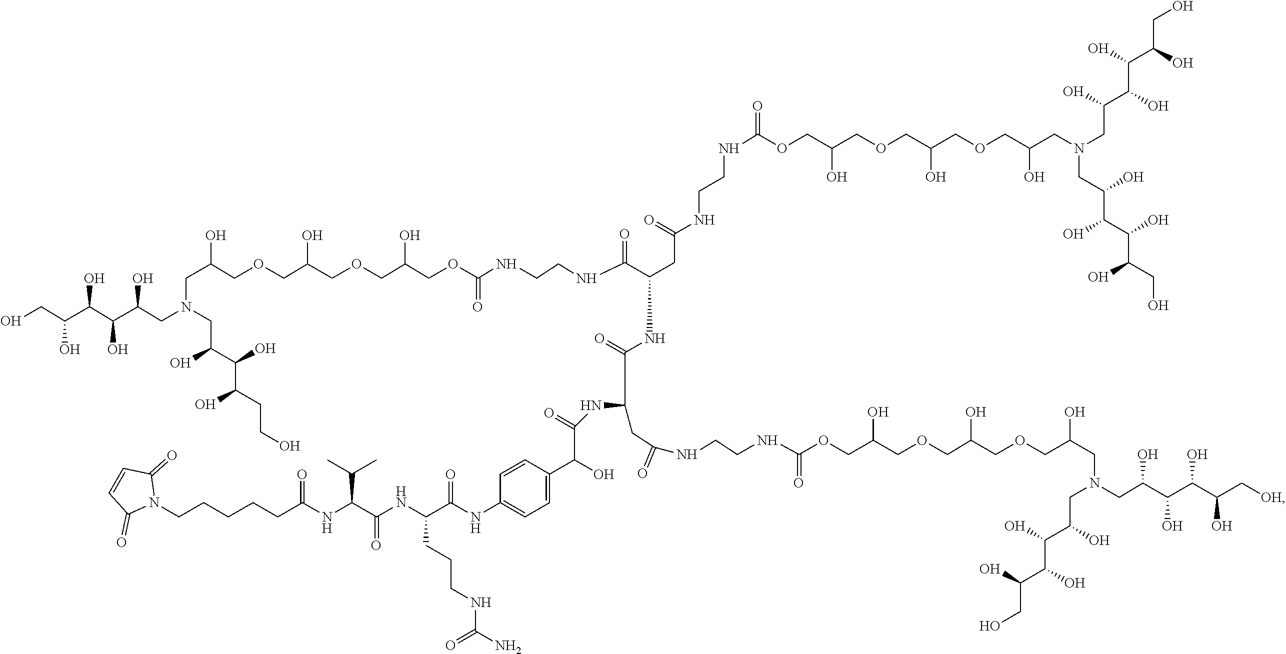 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)CCO)cc1]

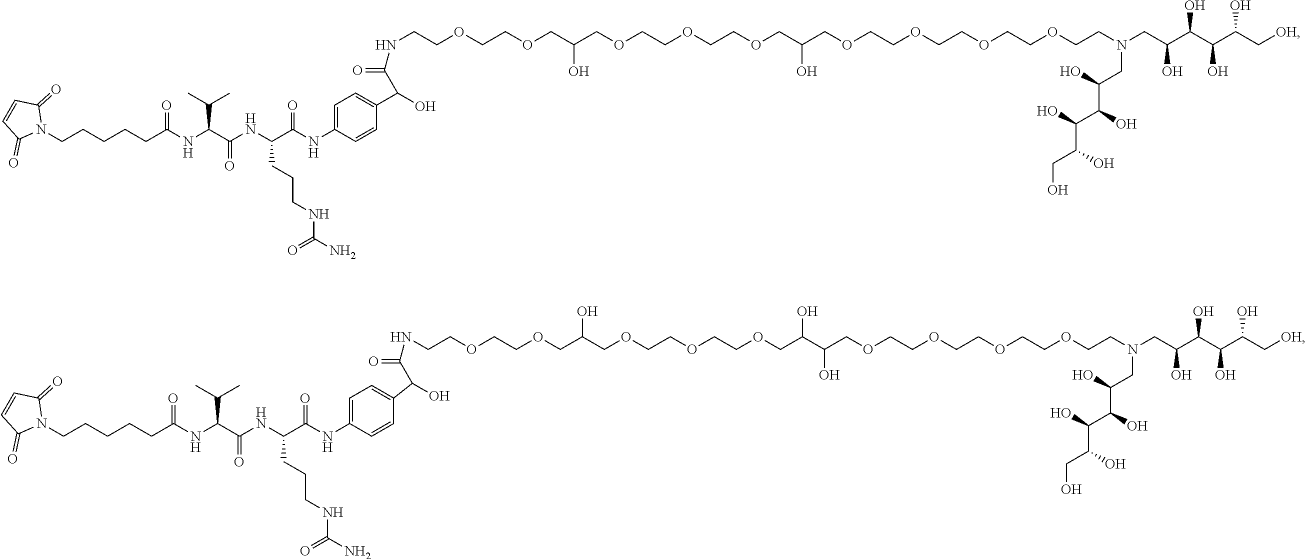 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)COCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)C(O)COCCOCCOCCOCCN(C[C@H](O)[C@@H](O)[C@H](O)[C@H](O)CO)C[C@H](O)[C@@H](O)[C@H](O)[C@H](O)CO)cc1]

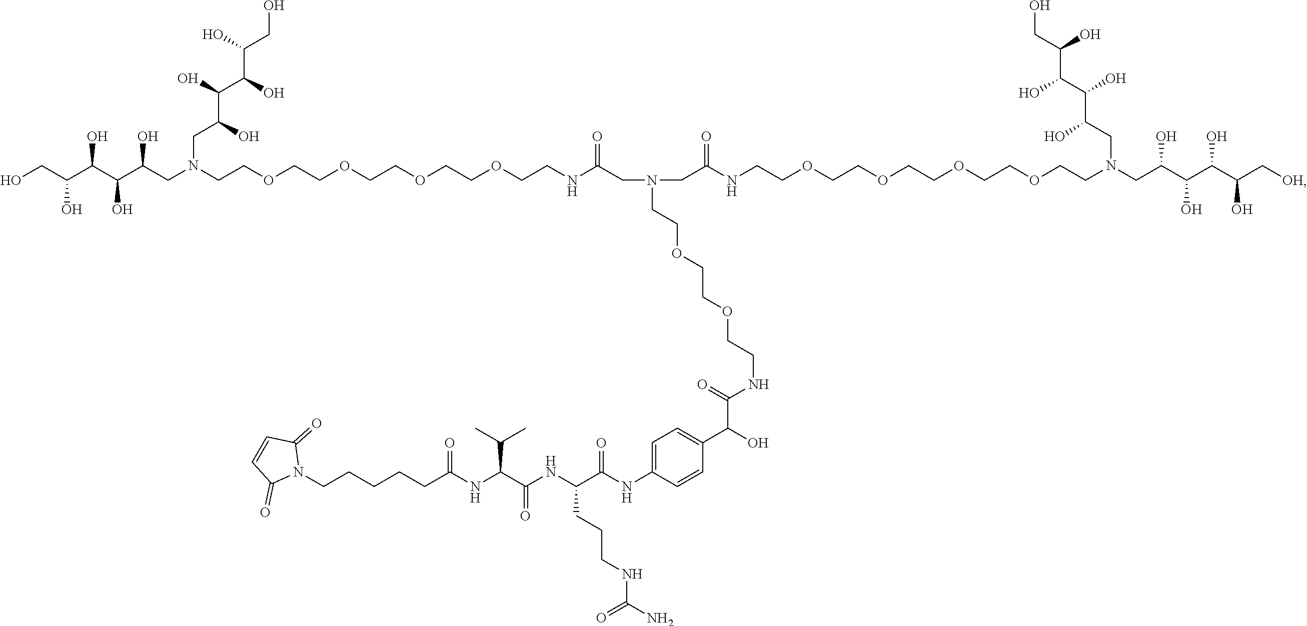 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

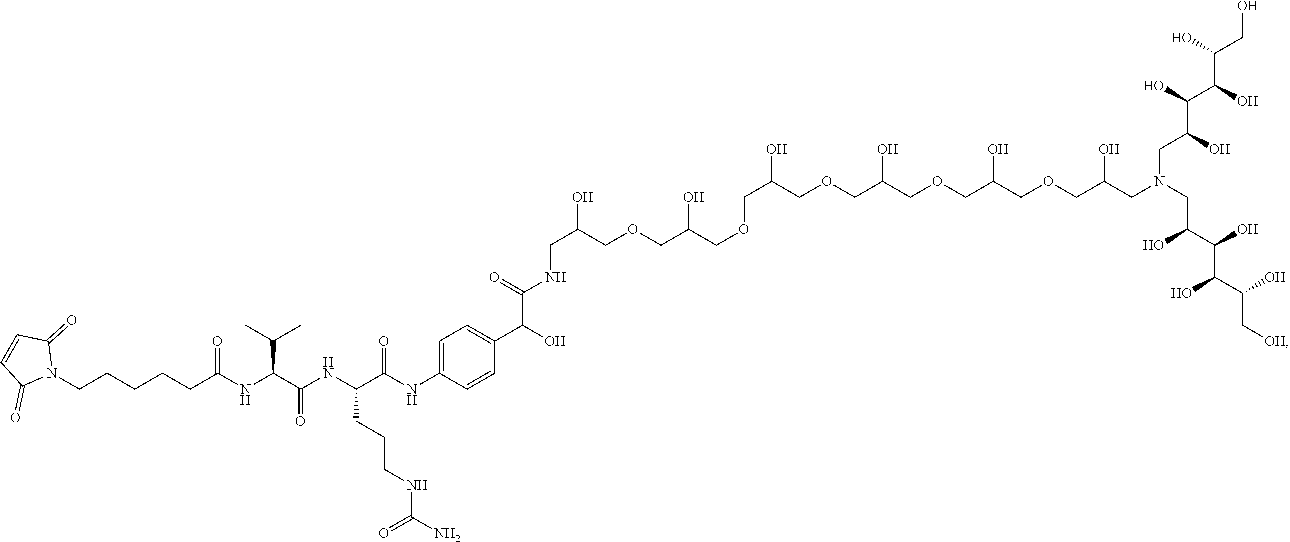 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

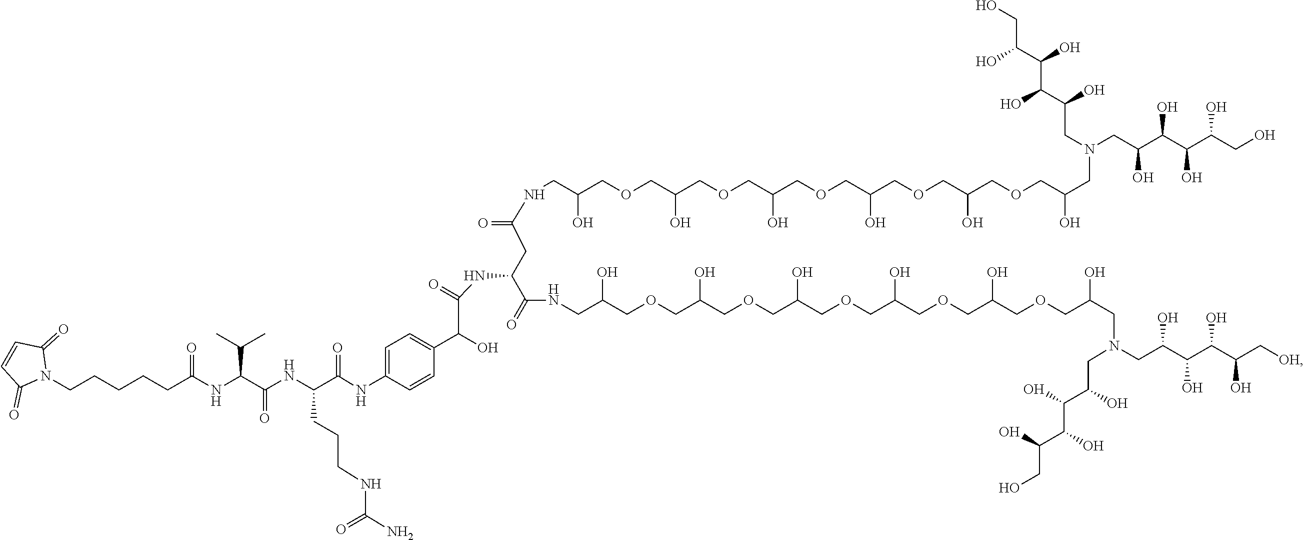 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

wherein the H on the benzylic OH is optionally replaced with a bond to the at least one drug unit or to the linking group attached to the at least one drug unit.

Embodiment 45. A drug-linker compound, comprising a linker compound of any of Embodiments 1-44 attached to the at least one drug unit, or attached to the linking group attached to the at least one drug unit, at the attachment site δ.

Embodiment 46. The drug-linker of Embodiment 45, wherein the drug unit is selected from a cytotoxic agent, an immune modulatory agent, a nucleic acid, a growth inhibitory agent, a PROTAC, a toxin, a radioactive isotope and a chelating ligand.

Embodiment 47. The drug-linker of Embodiment 46, wherein the drug unit is a cytotoxic agent.

Embodiment 48. The drug-linker of Embodiment 47, wherein the cytotoxic agent is selected from the group consisting of an auristatin, a maytansinoid, a camptothecin, a duocarmycin, and a calicheamicin.

Embodiment 49. The drug-linker of Embodiment 48, wherein the cytotoxic agent is an auristatin.

Embodiment 50. The drug-linker of Embodiment 48, wherein the cytotoxic agent is a camptothecin.

Embodiment 51. The drug-linker of Embodiment 50, wherein the cytotoxic agent is RS-exatecan or SS-exatecan.

Embodiment 52. The drug-linker of Embodiment 51, wherein the maytansinoid is maytansine, maytansinol or ansamatocin-2.

Embodiment 53. The drug-linker of Embodiment 46, wherein the drug unit is an immune modulatory agent.

Embodiment 54. The drug-linker of Embodiment 53, wherein the immune modulatory agent is selected from a TRL7 agonist, a TLR8 agonist, a STING agonist, or a RIG-1 agonist.

Embodiment 55. The drug-linker of Embodiment 54, wherein the immune modulatory agent is an TLR7 agonist.

Embodiment 56. The drug-linker of Embodiment 55, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

Embodiment 57. The drug-linker of Embodiment 54, wherein the immune modulatory agent is a TLR8 agonist.

Embodiment 58. The drug-linker of Embodiment 57, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

Embodiment 59. The drug-linker of Embodiment 53, wherein the immune modulatory agent is a STING agonist.

Embodiment 60. The drug-linker of Embodiment 54, wherein the immune modulatory agent is a RIG-I agonist.

Embodiment 61. The drug-linker of Embodiment 60, wherein the RIG-1 agonist is selected from KIN1148, SB-9200, KIN700, KIN600, KIN500, KIN100, KIN101, KIN400 and KIN2000.

Embodiment 62. The drug-linker of Embodiment 46, wherein the drug unit is a chelating ligand.

Embodiment 63. The drug-linker of Embodiment 62, wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

Embodiment 64. The drug-linker of Embodiment 45, having one of the following structure:

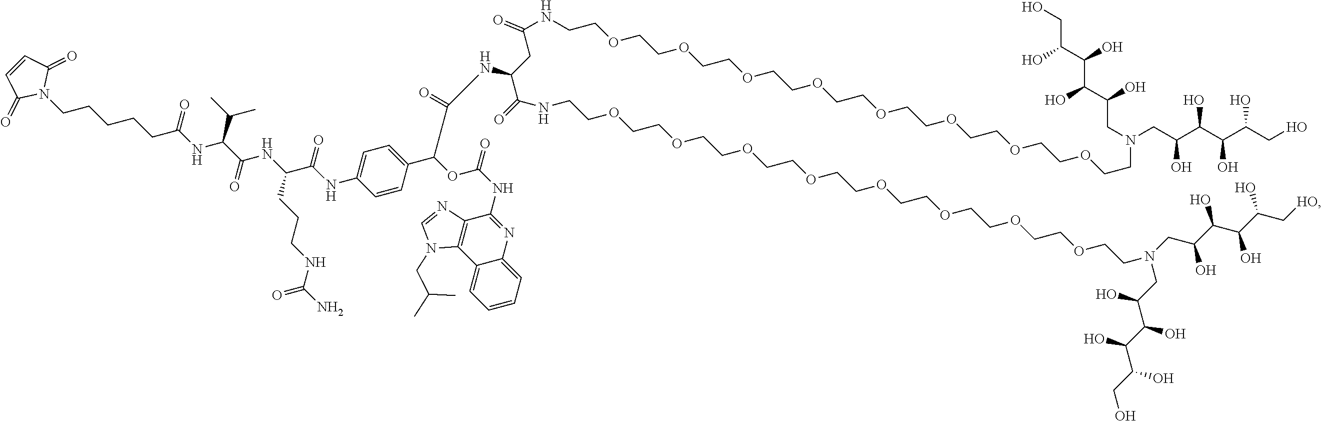 [SMILES: CC(C)Cn1cnc2c(NC(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c3ccc(NC(=O)[C@H](CCCNC(N)=O)NC(=O)[C@@H](NC(=O)CCCCCN4C(=O)C=CC4=O)C(C)C)cc3)nc3ccccc3c21]

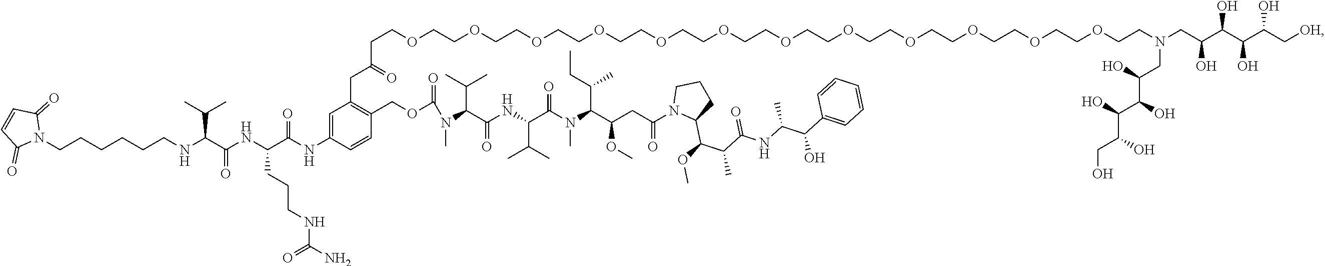 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NCCCCCCN2C(=O)C=CC2=O)C(C)C)cc1CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(C)C]

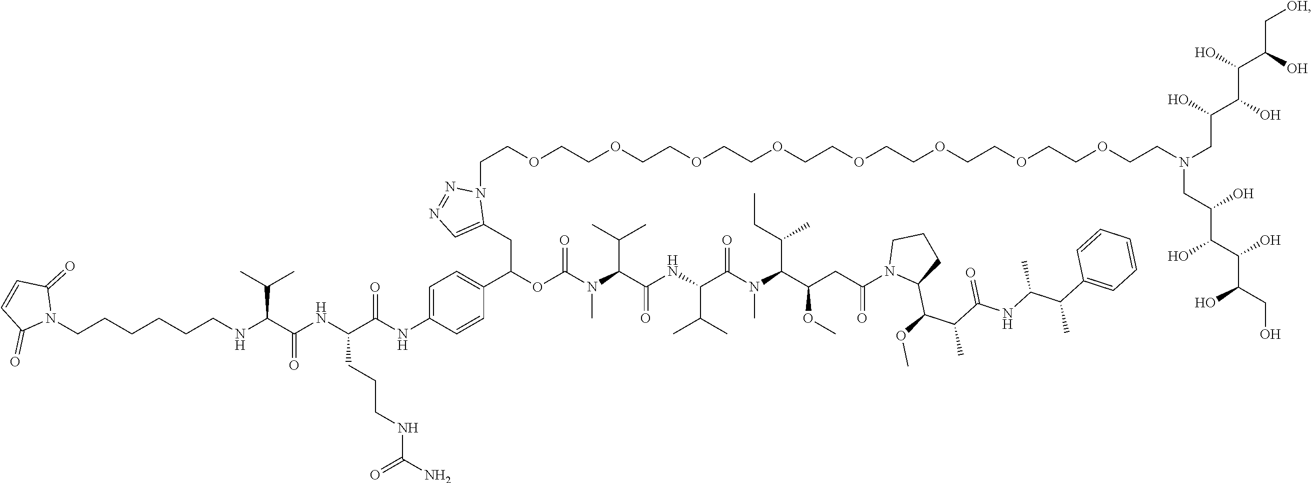 [SMILES: CC[C@H](C)[C@@H]([C@@H](CC(=O)N1CCC[C@H]1[C@H](OC)[C@@H](C)C(=O)N[C@H](C)[C@@H](C)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

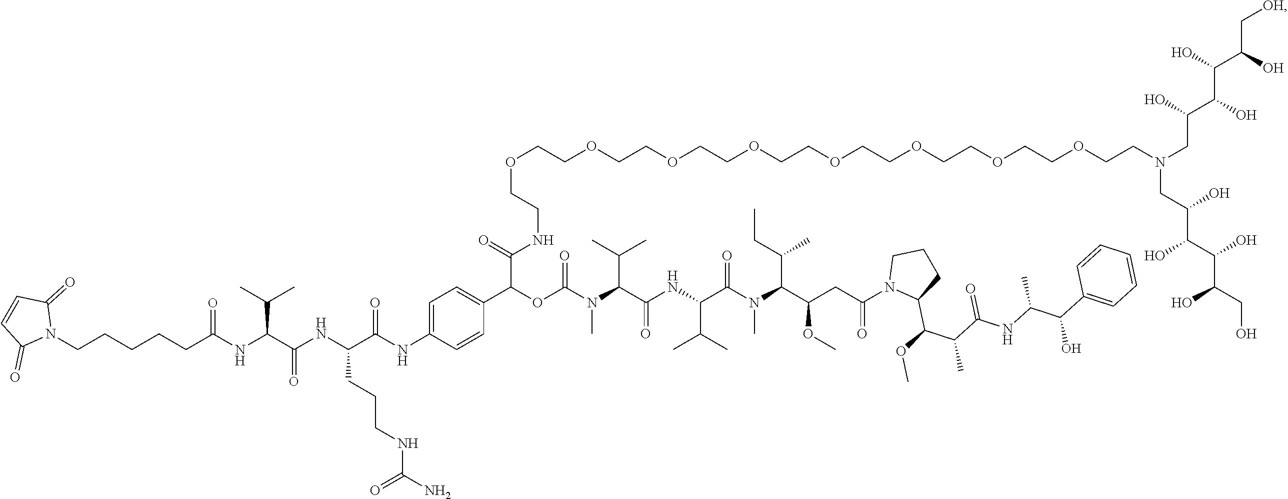 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

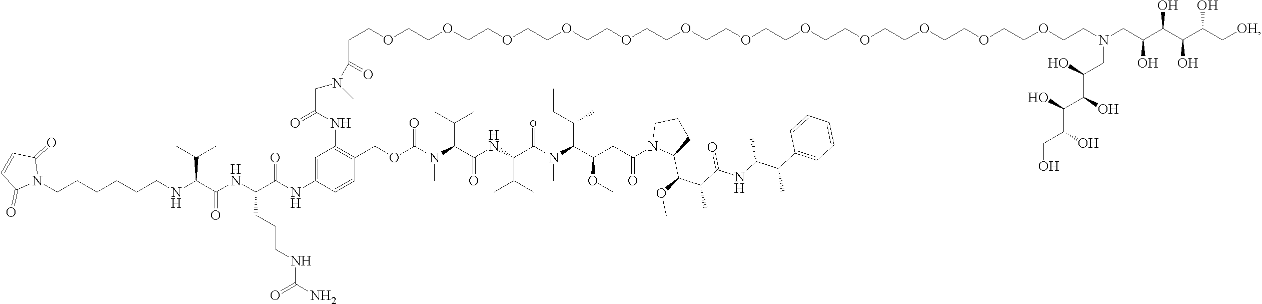 [SMILES: CC[C@H](C)[C@@H]([C@@H](CC(=O)N1CCC[C@H]1[C@H](OC)[C@@H](C)C(=O)N[C@H](C)[C@@H](C)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NCCCCCCN2C(=O)C=CC2=O)C(C)C)cc1NC(=O)CN(C)C(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(C)C]

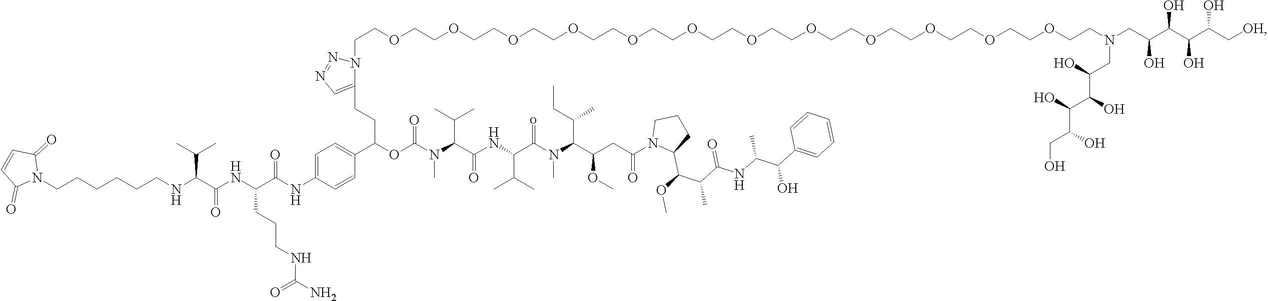 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(CCc1cnnn1CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

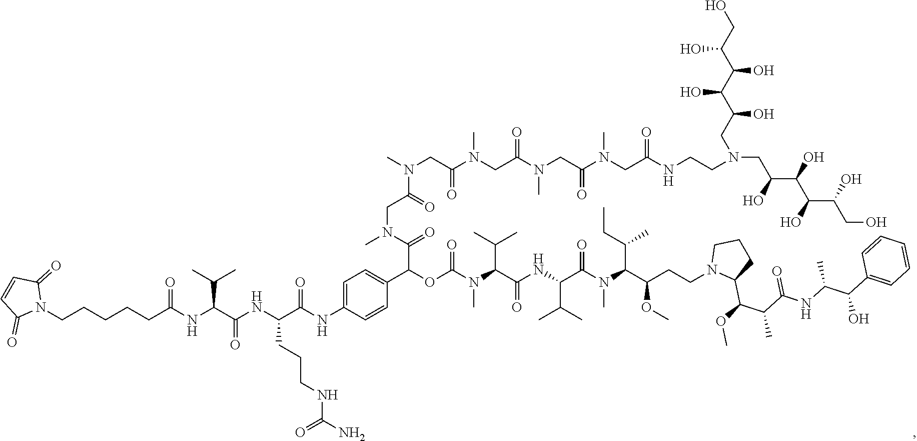 [SMILES: CC[C@H](C)[C@@H]([C@@H](CCN1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

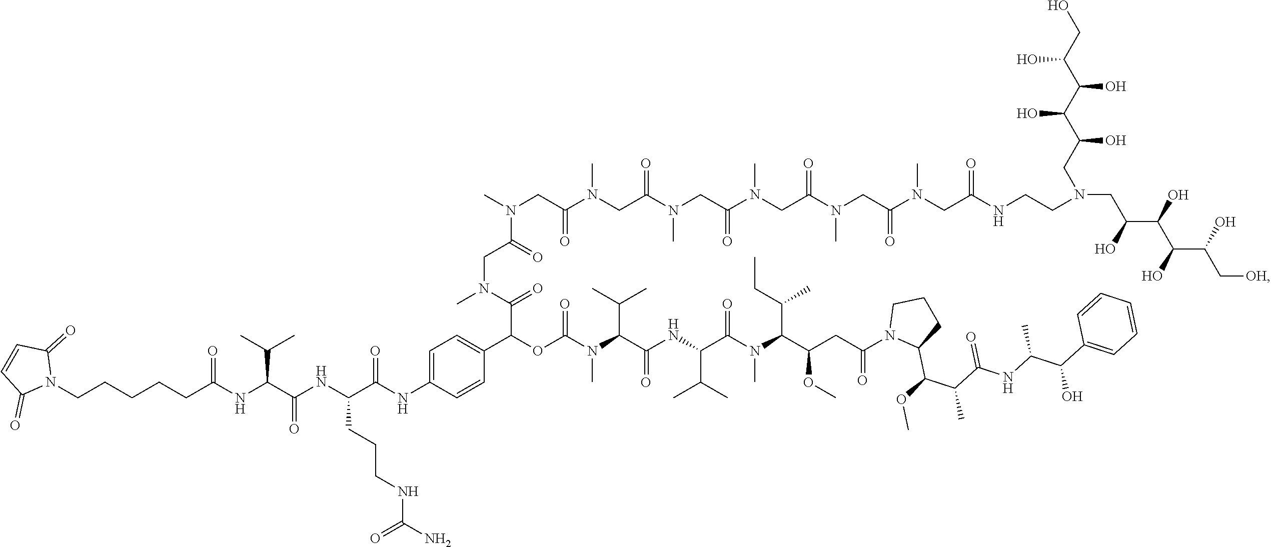 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

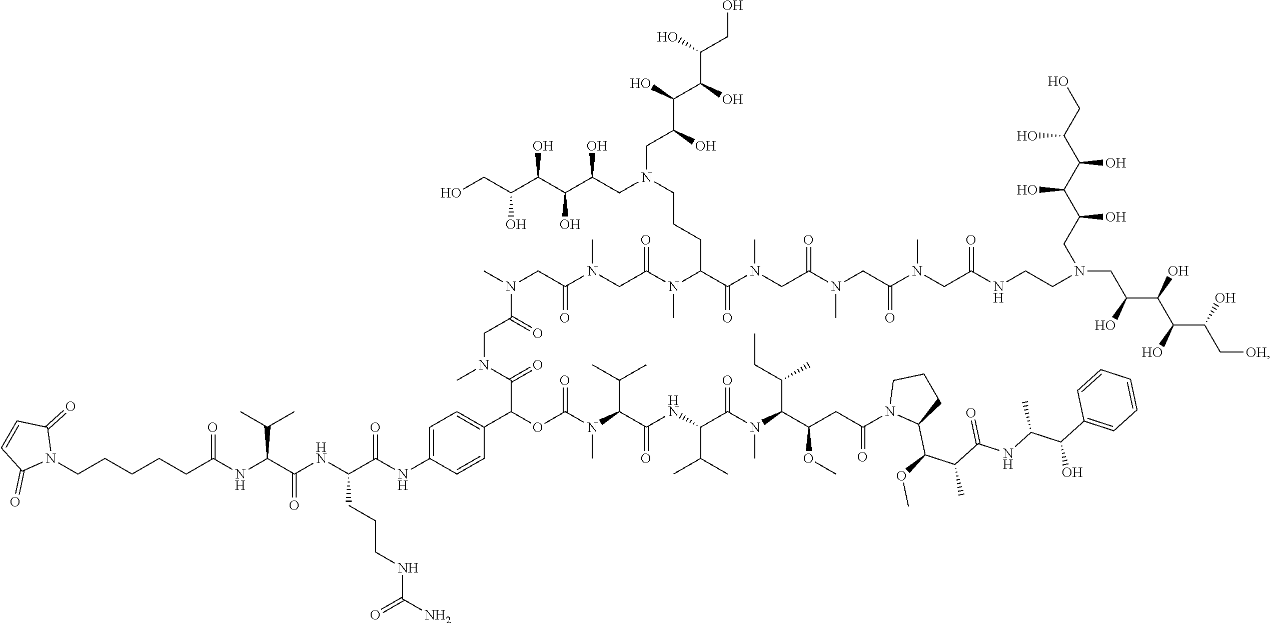 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

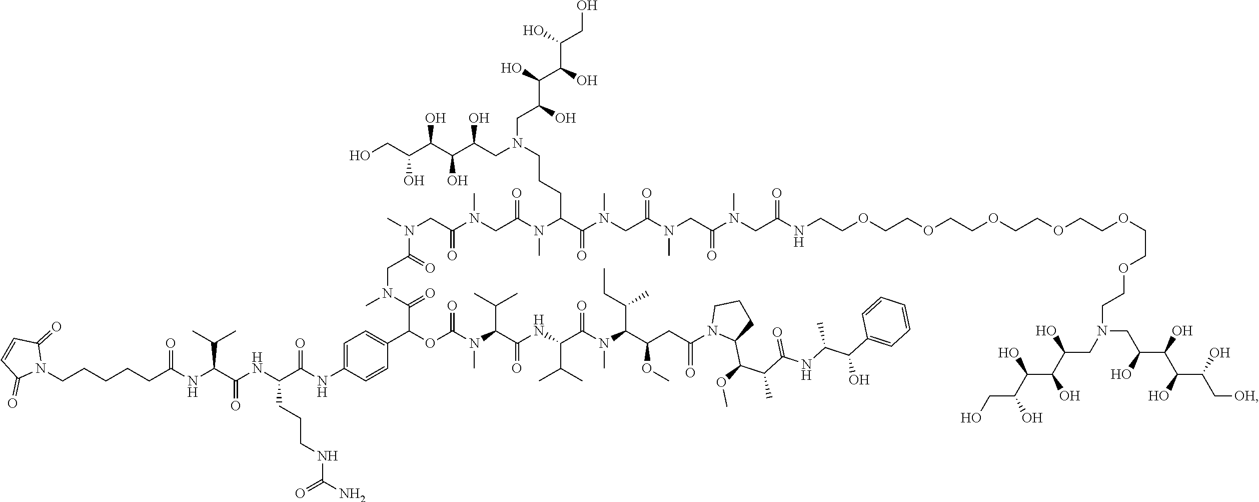 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

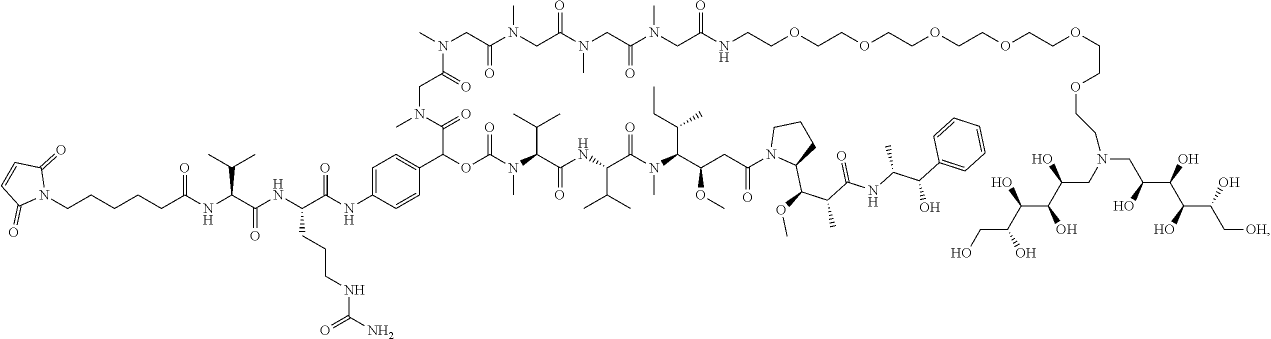 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

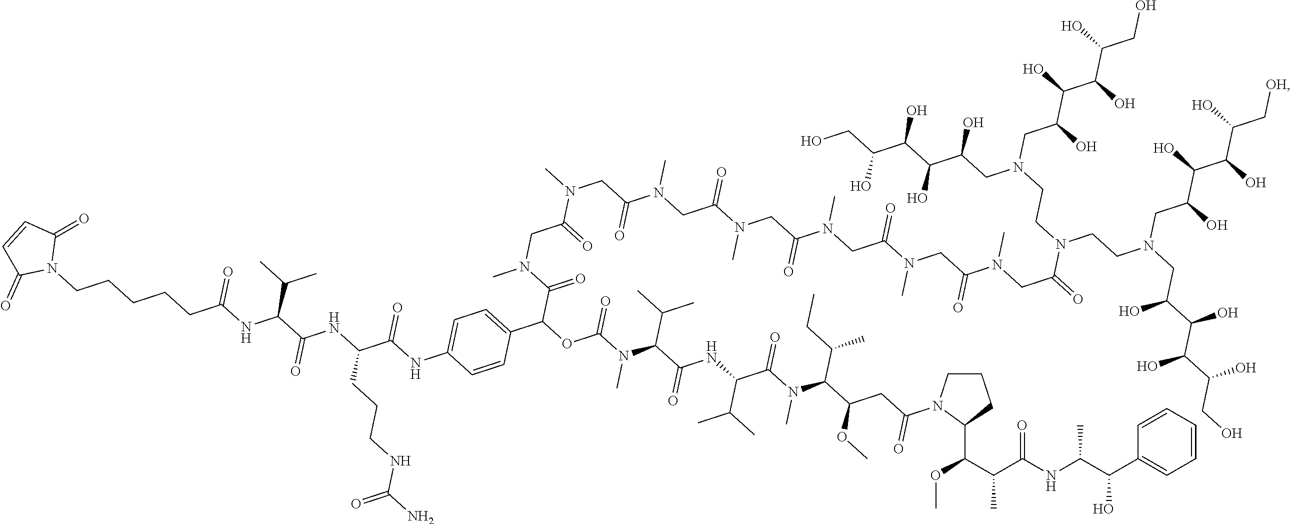 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

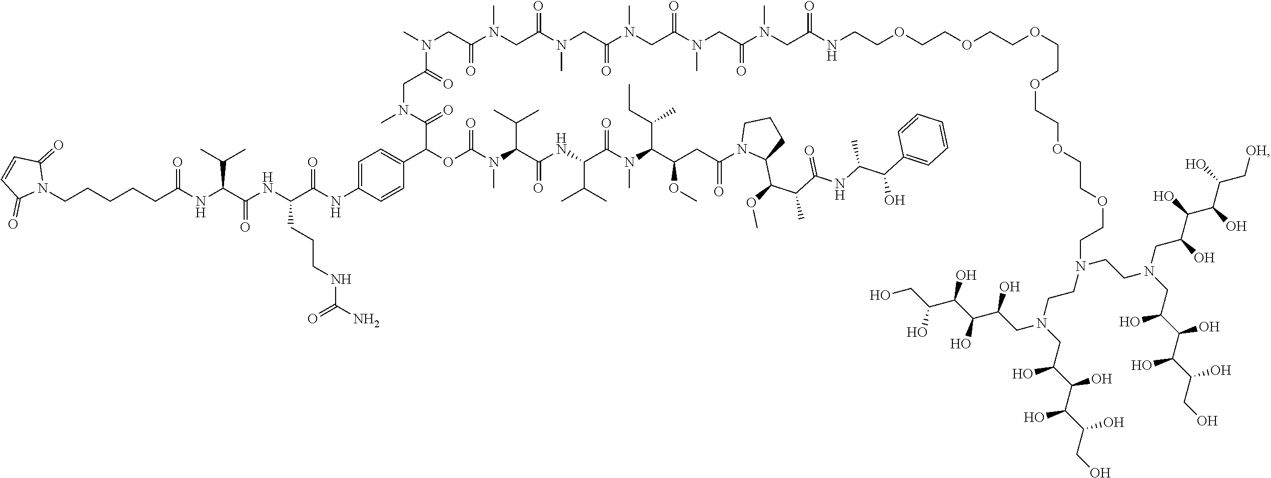 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

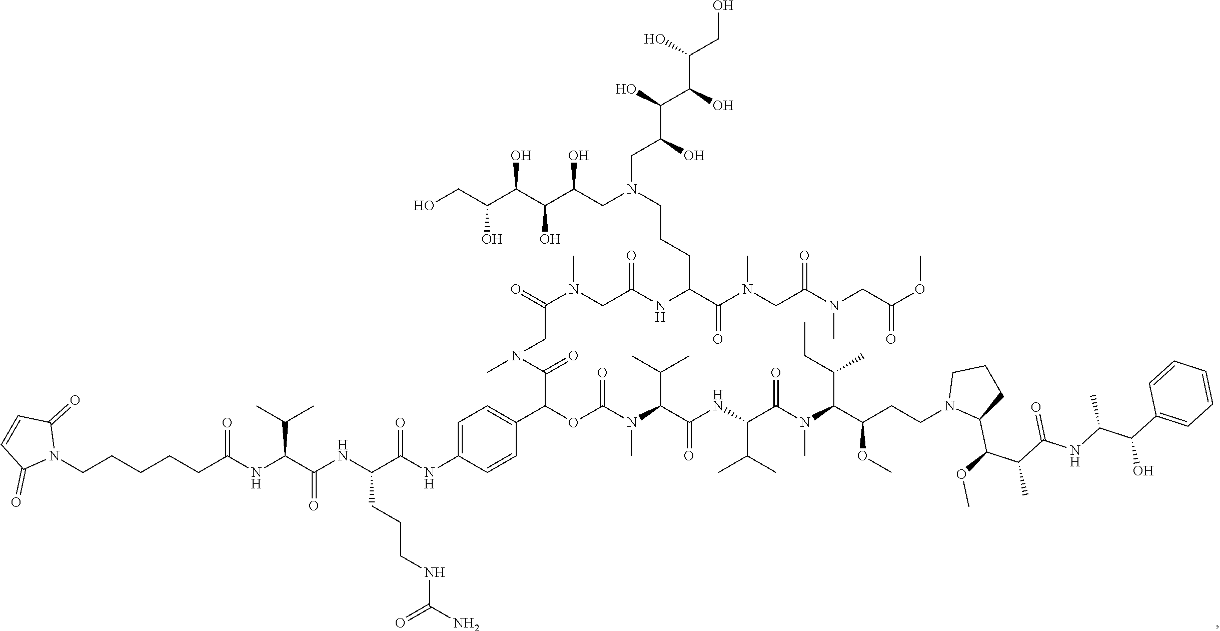 [SMILES: CC[C@H](C)[C@@H]([C@@H](CCN1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

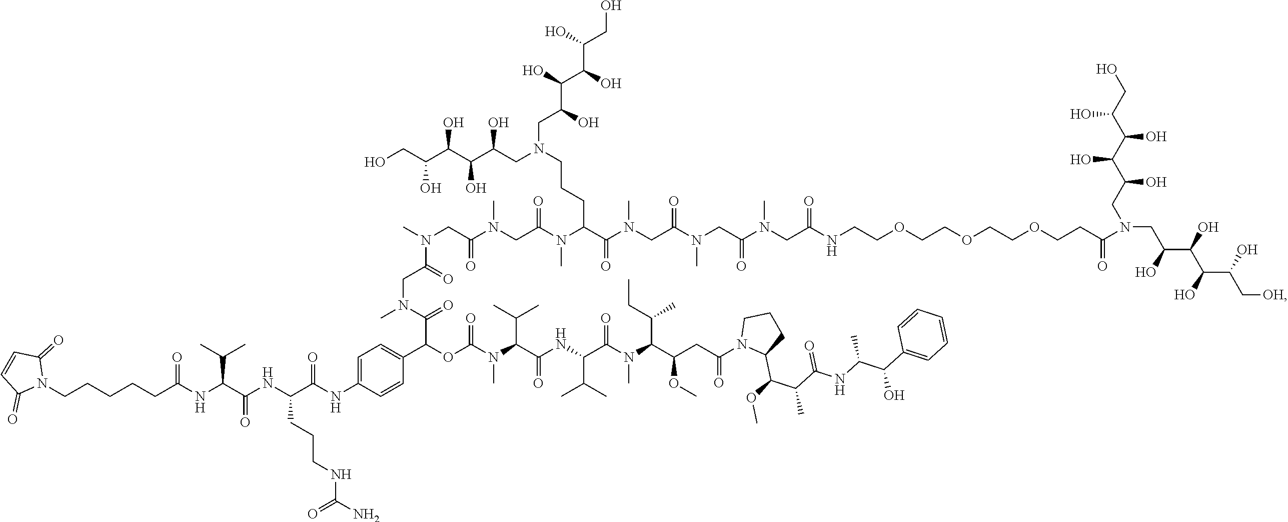 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

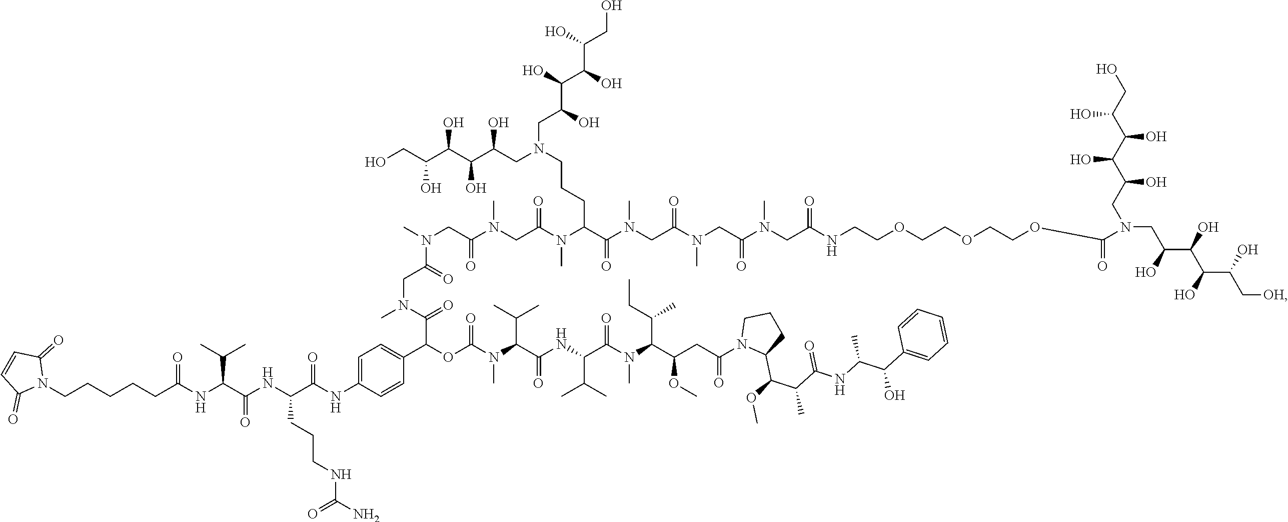 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

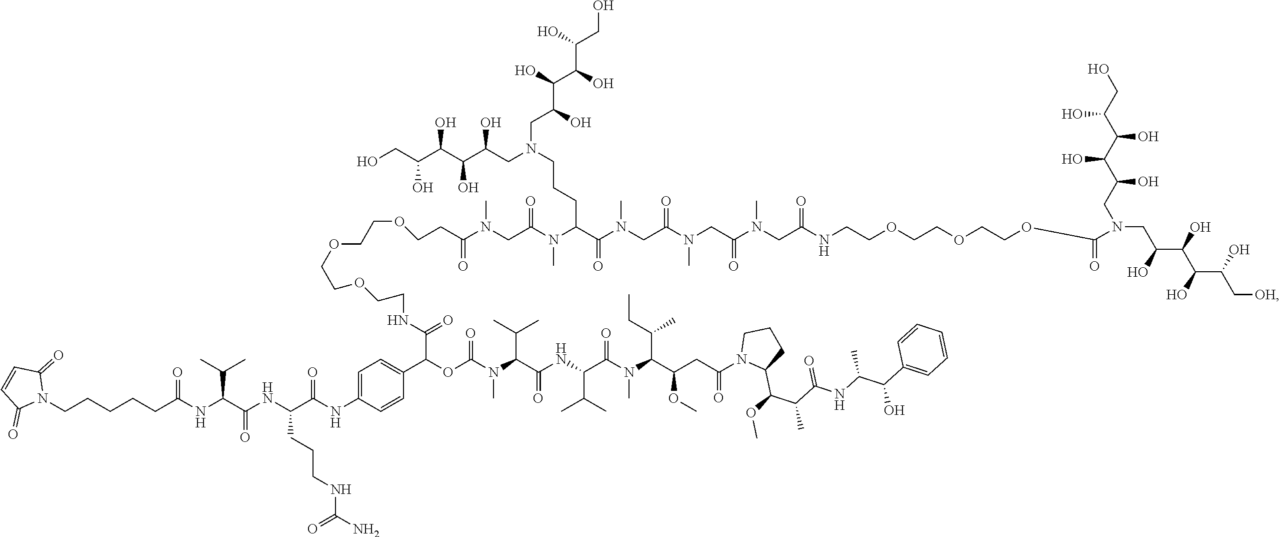 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

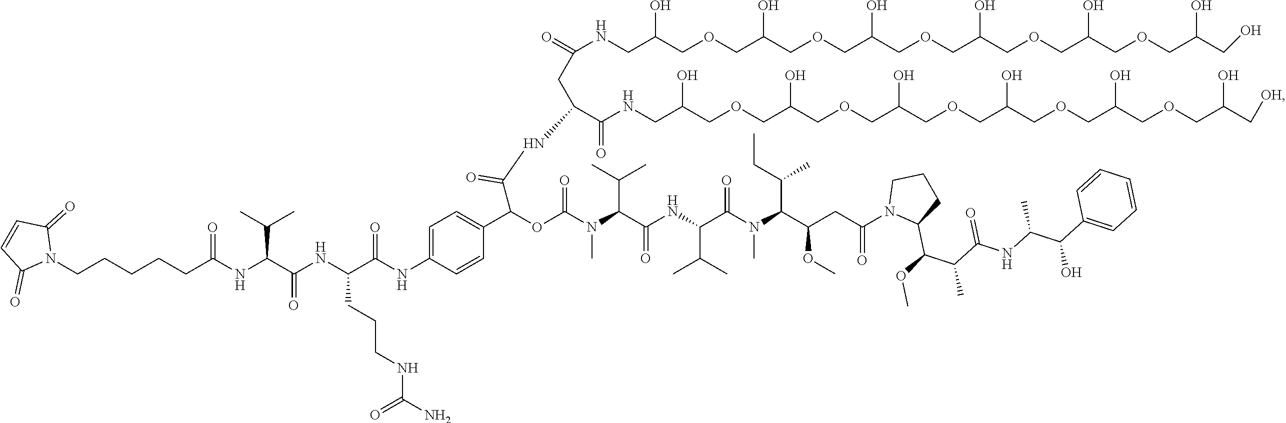 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

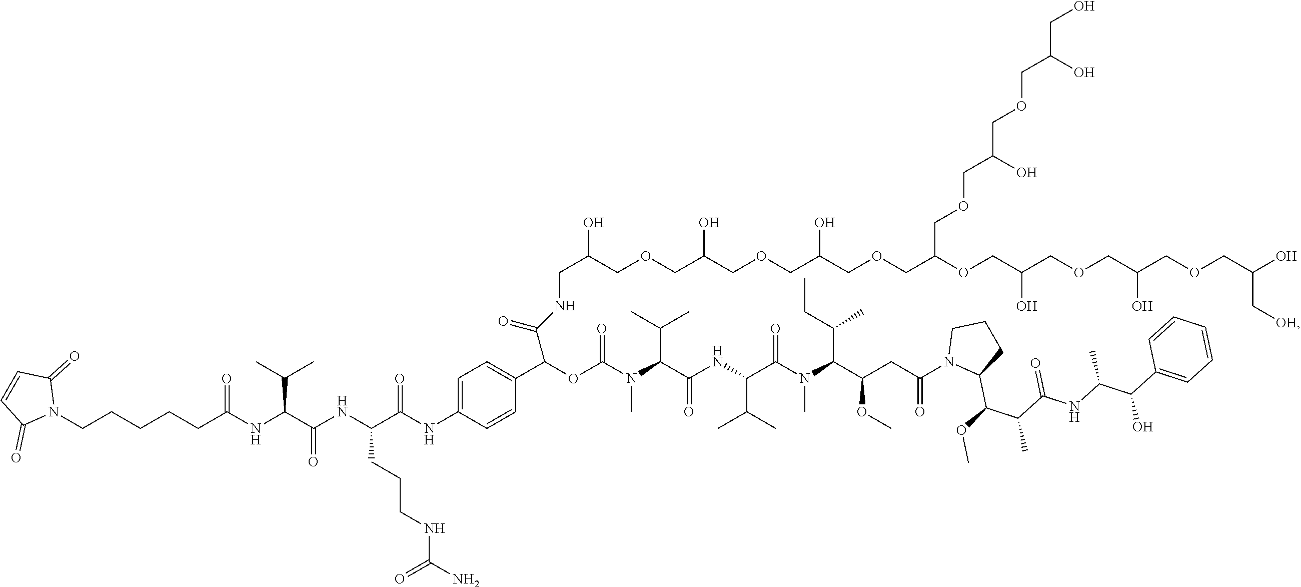 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(COCC(O)COCC(O)CO)OCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

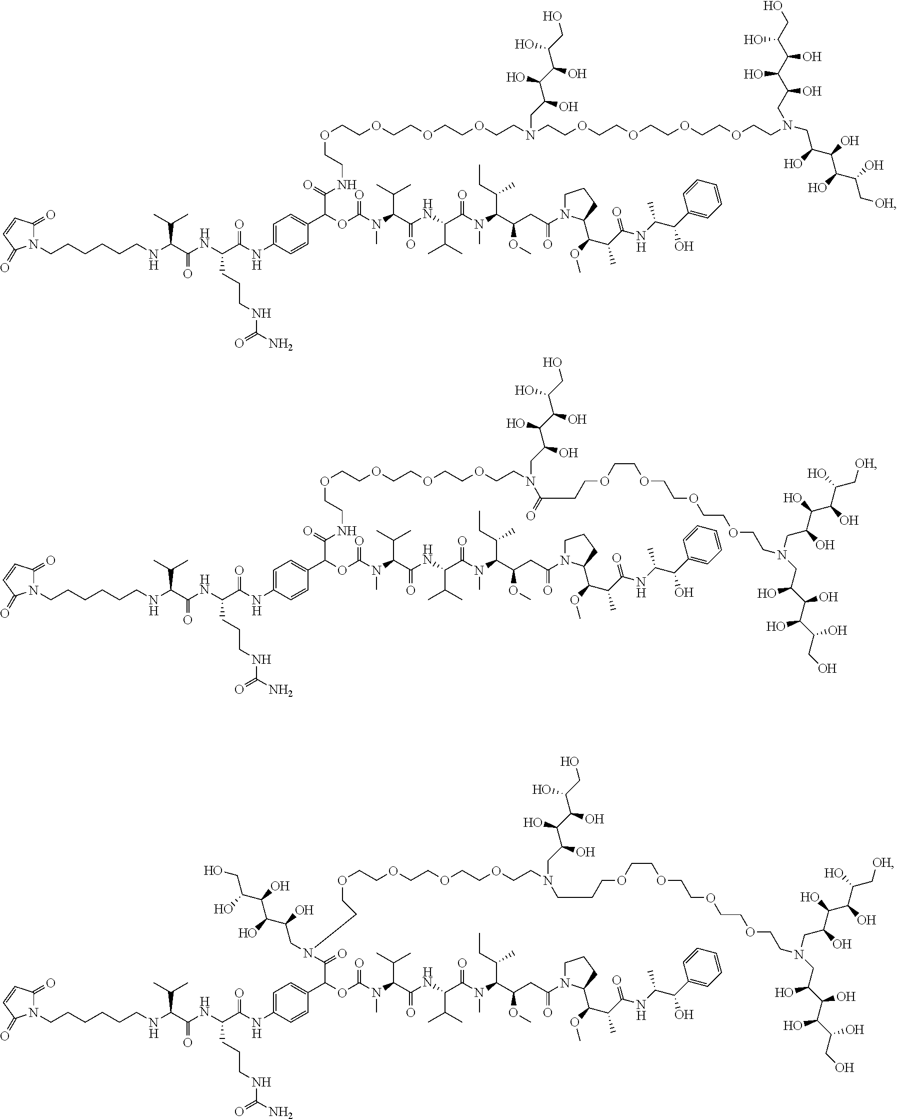

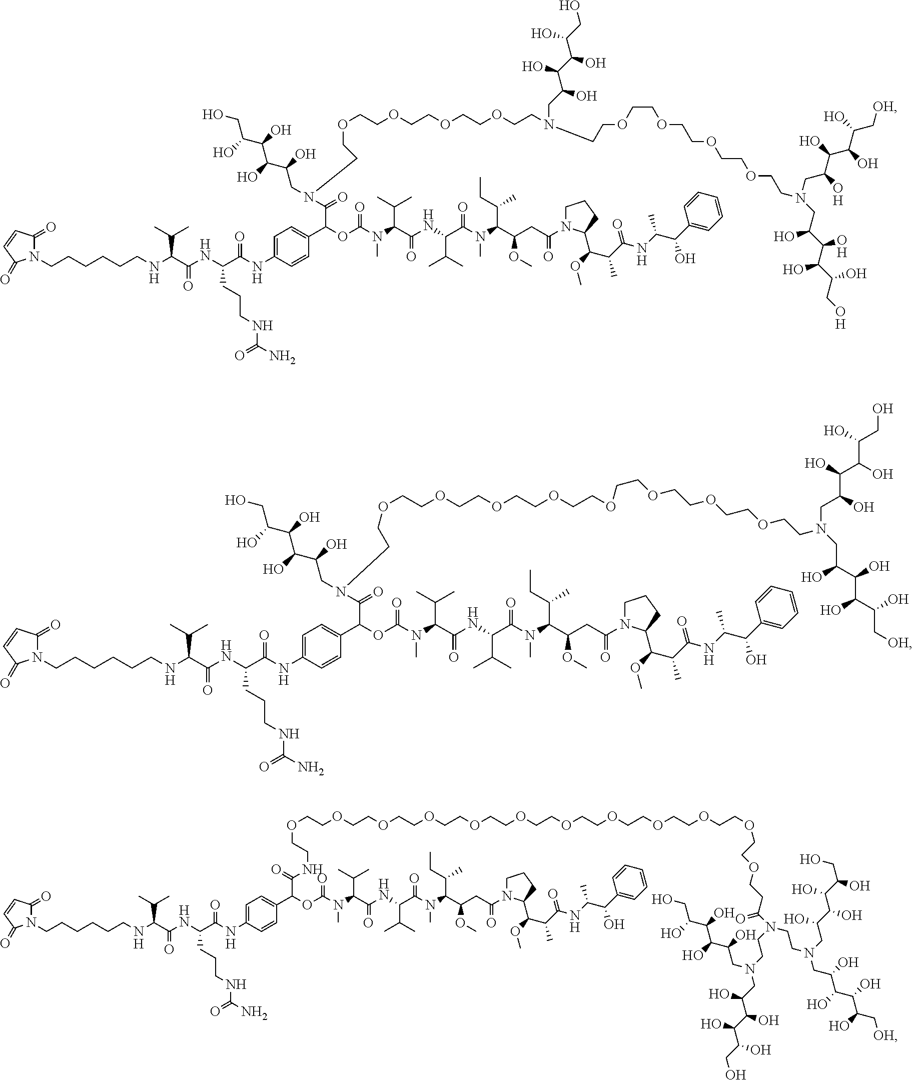

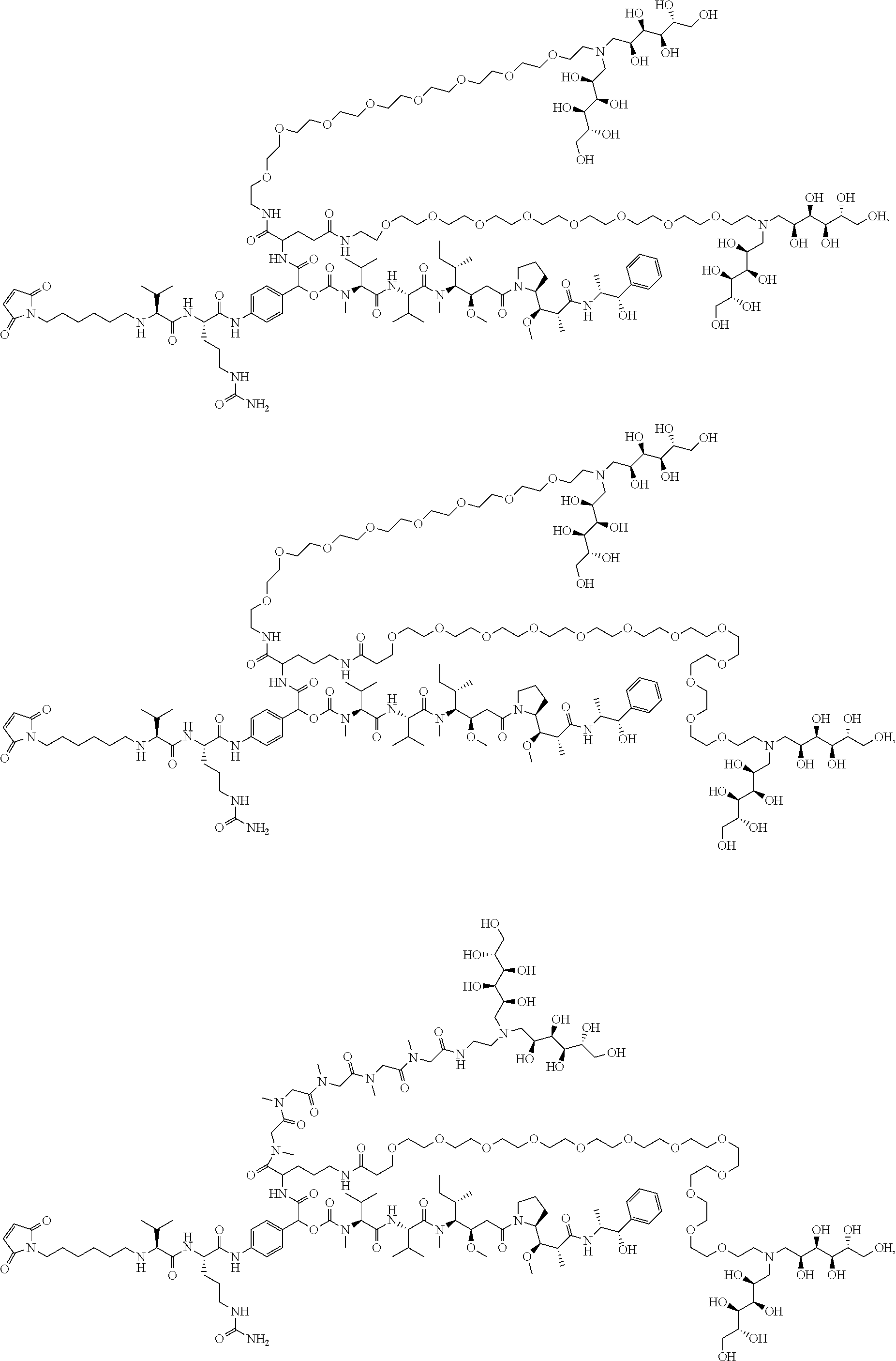

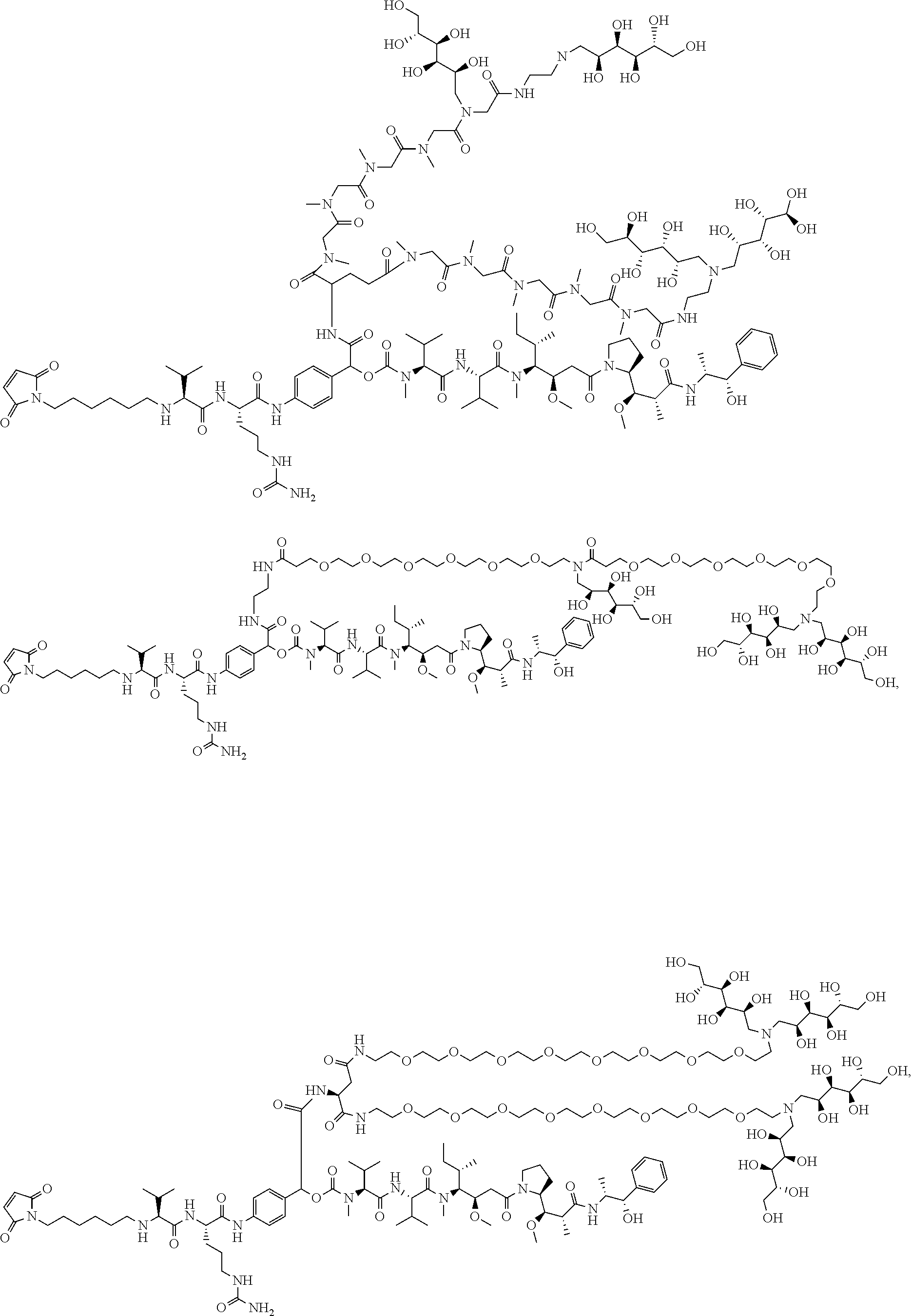

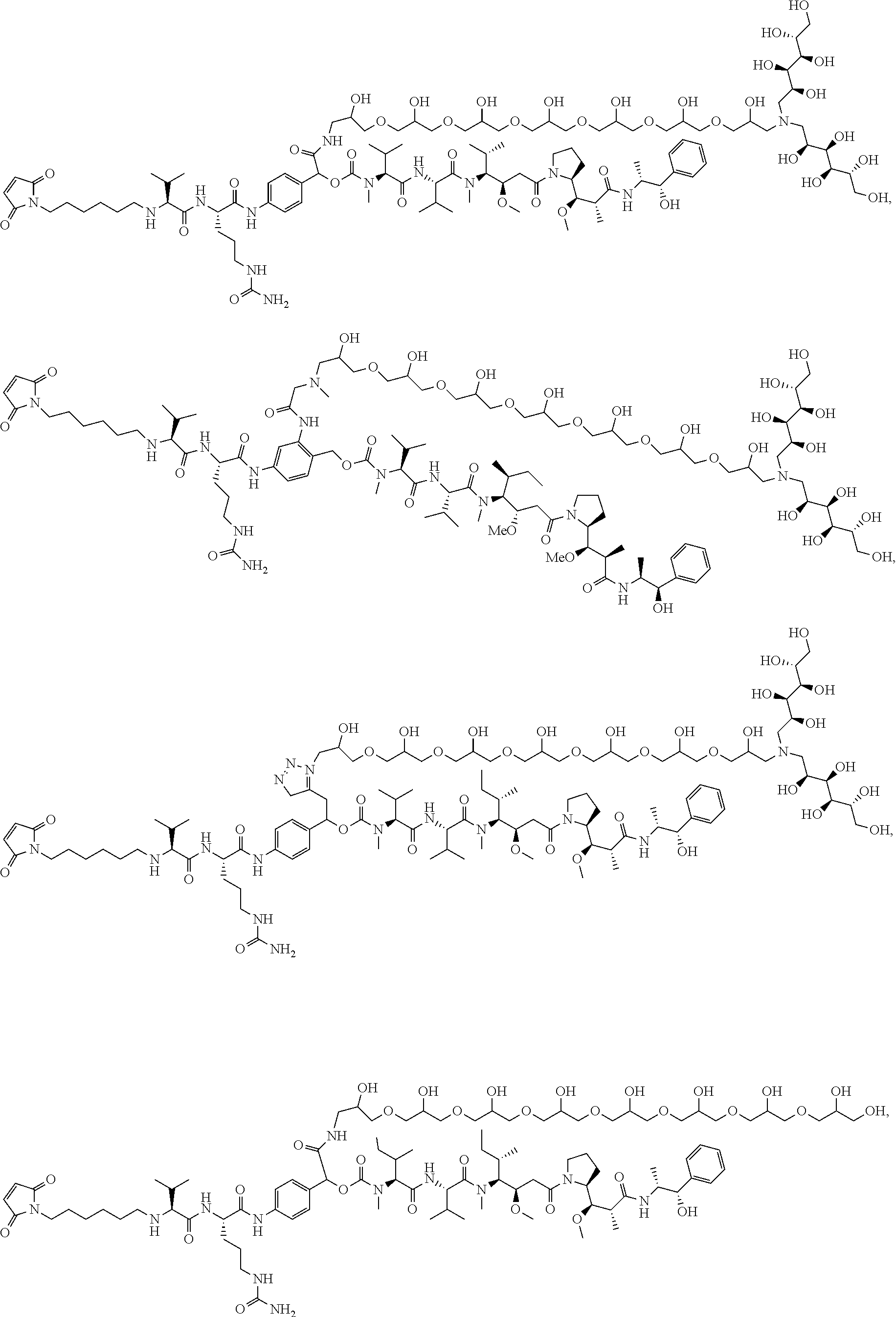

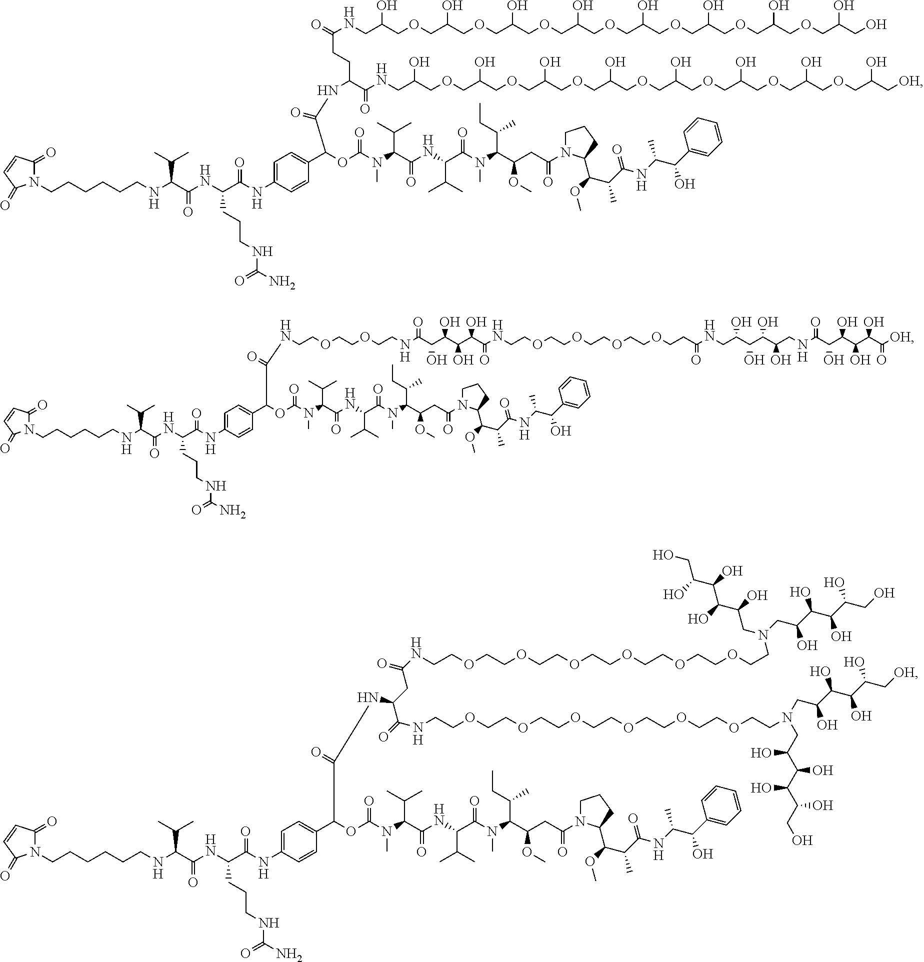

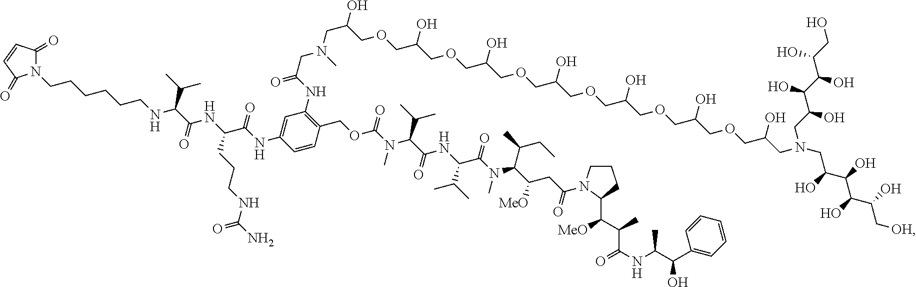 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NCCCCCCN2C(=O)C=CC2=O)C(C)C)cc1NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(C)C]

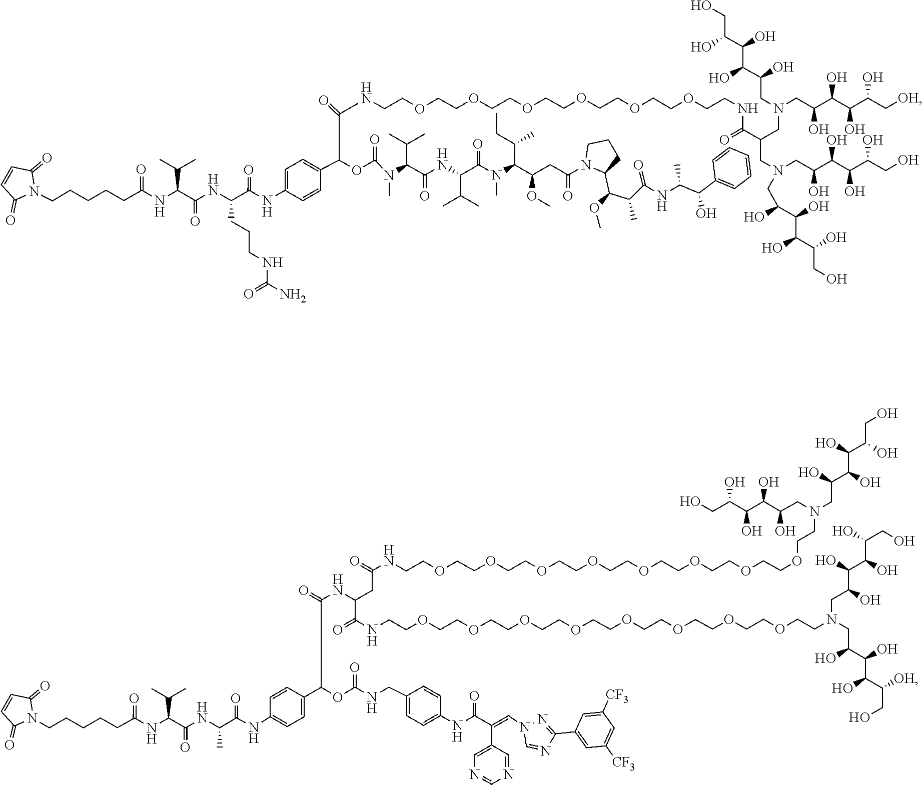

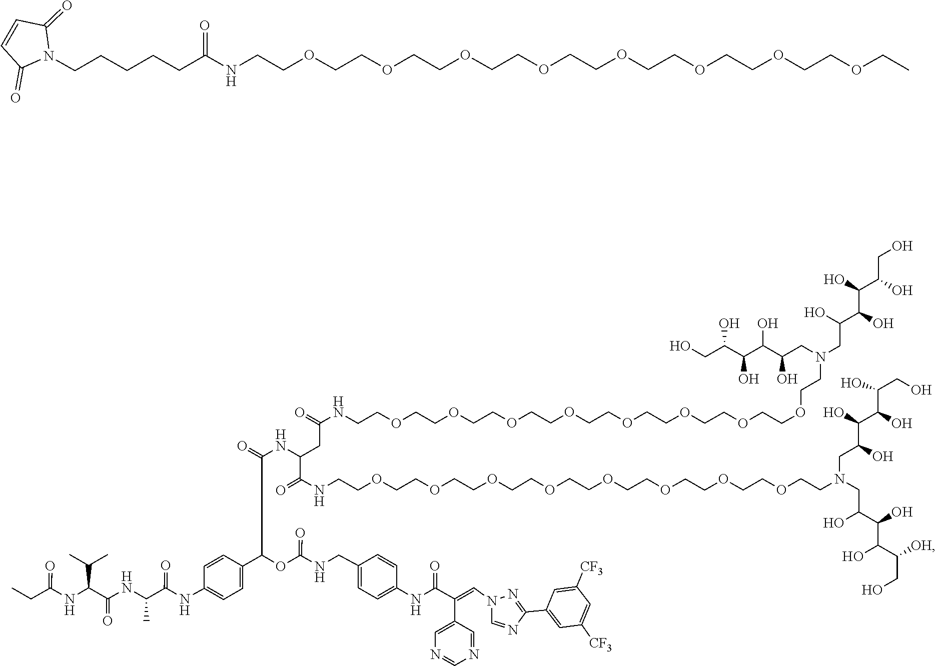

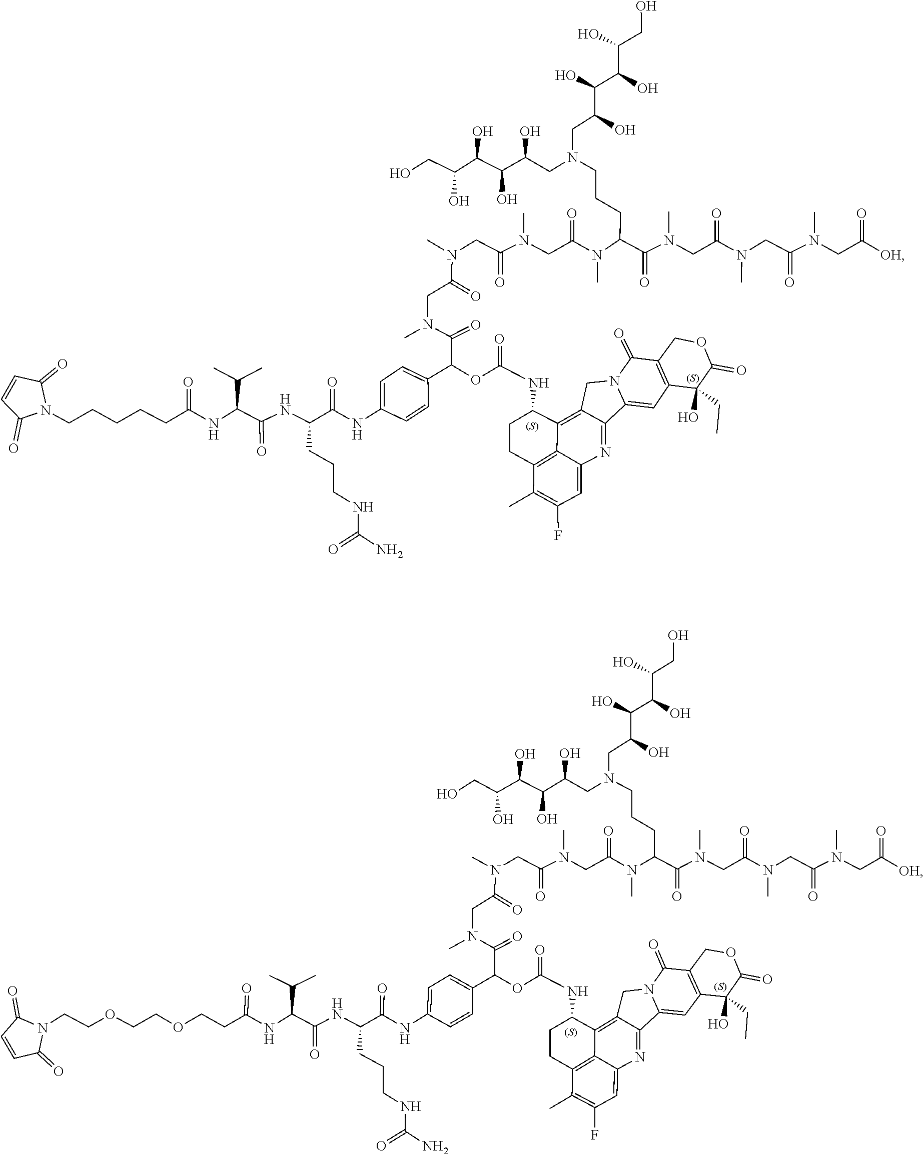

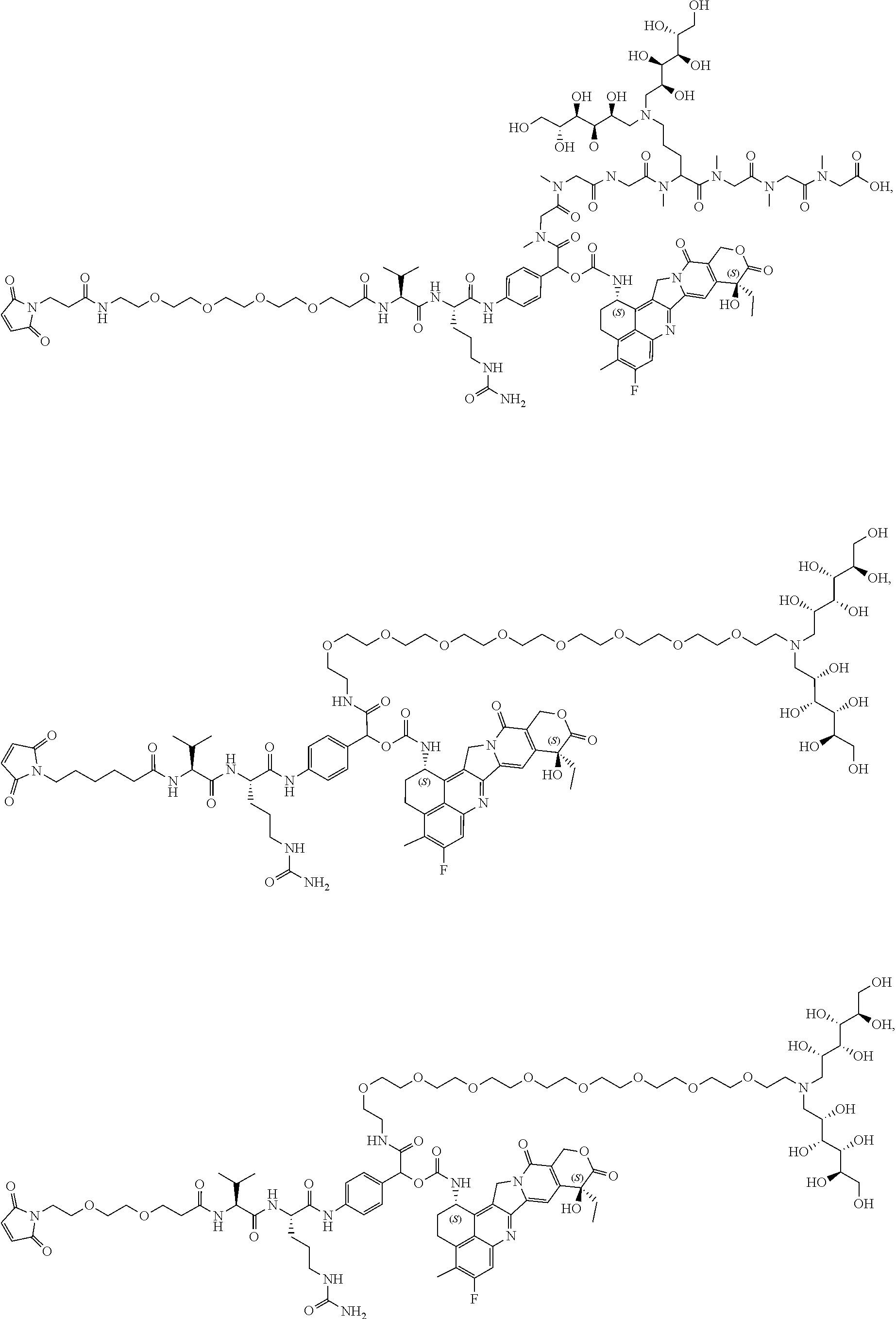

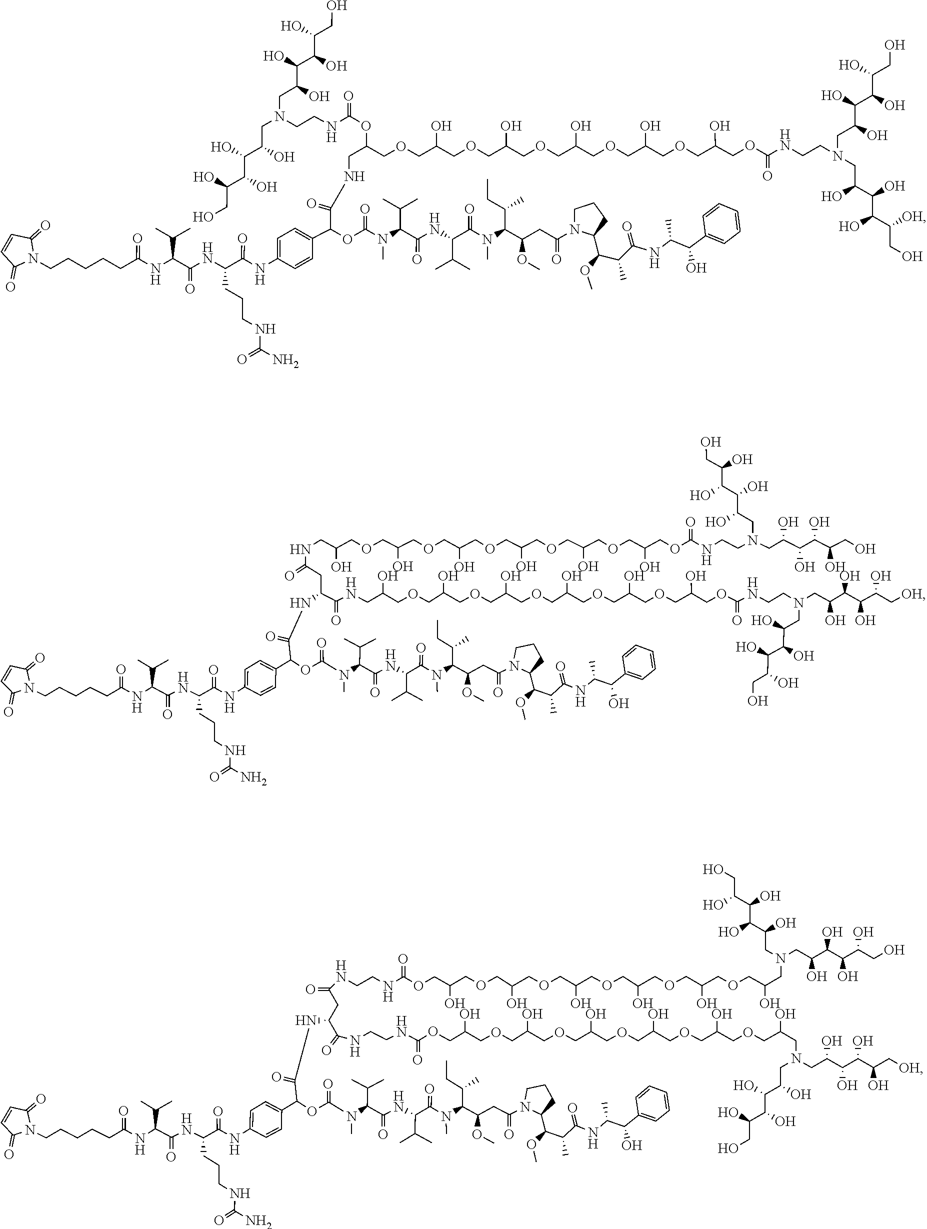

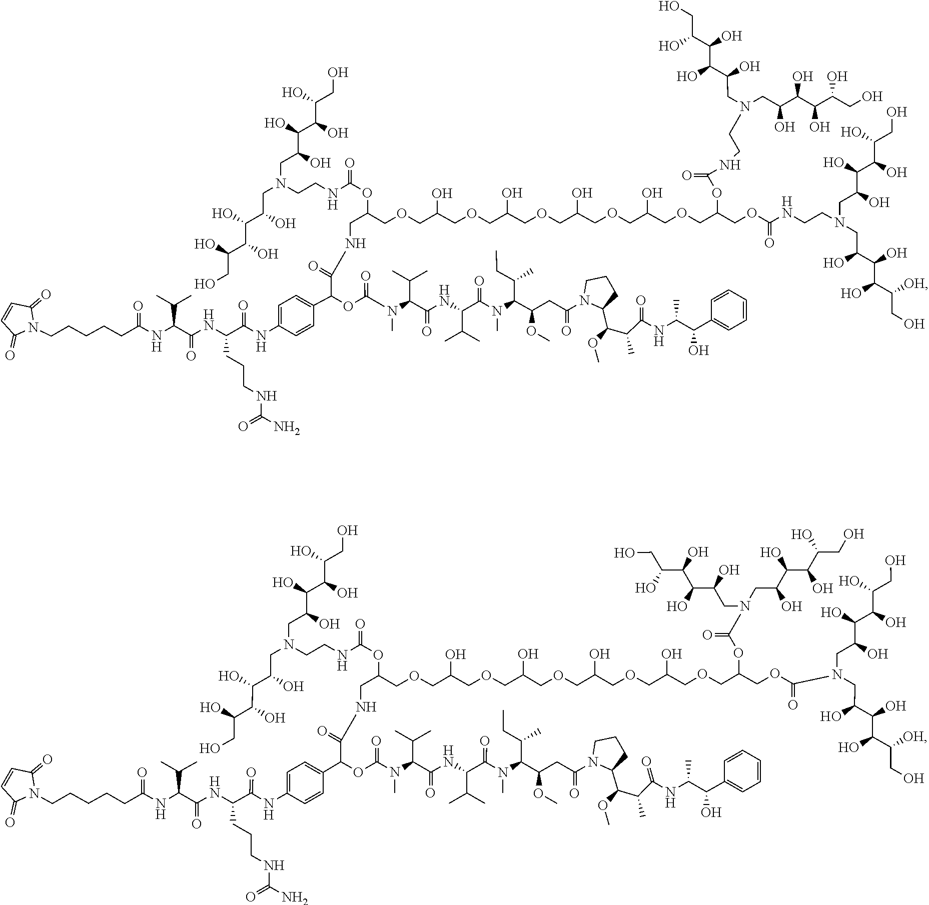

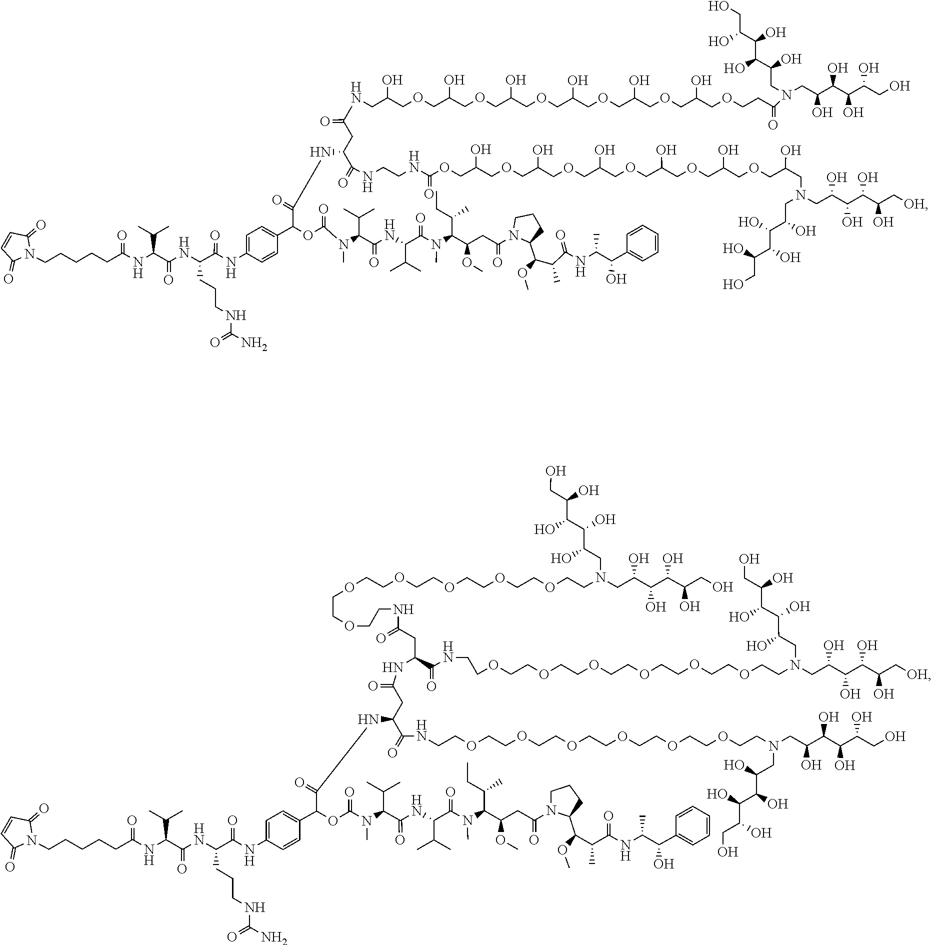

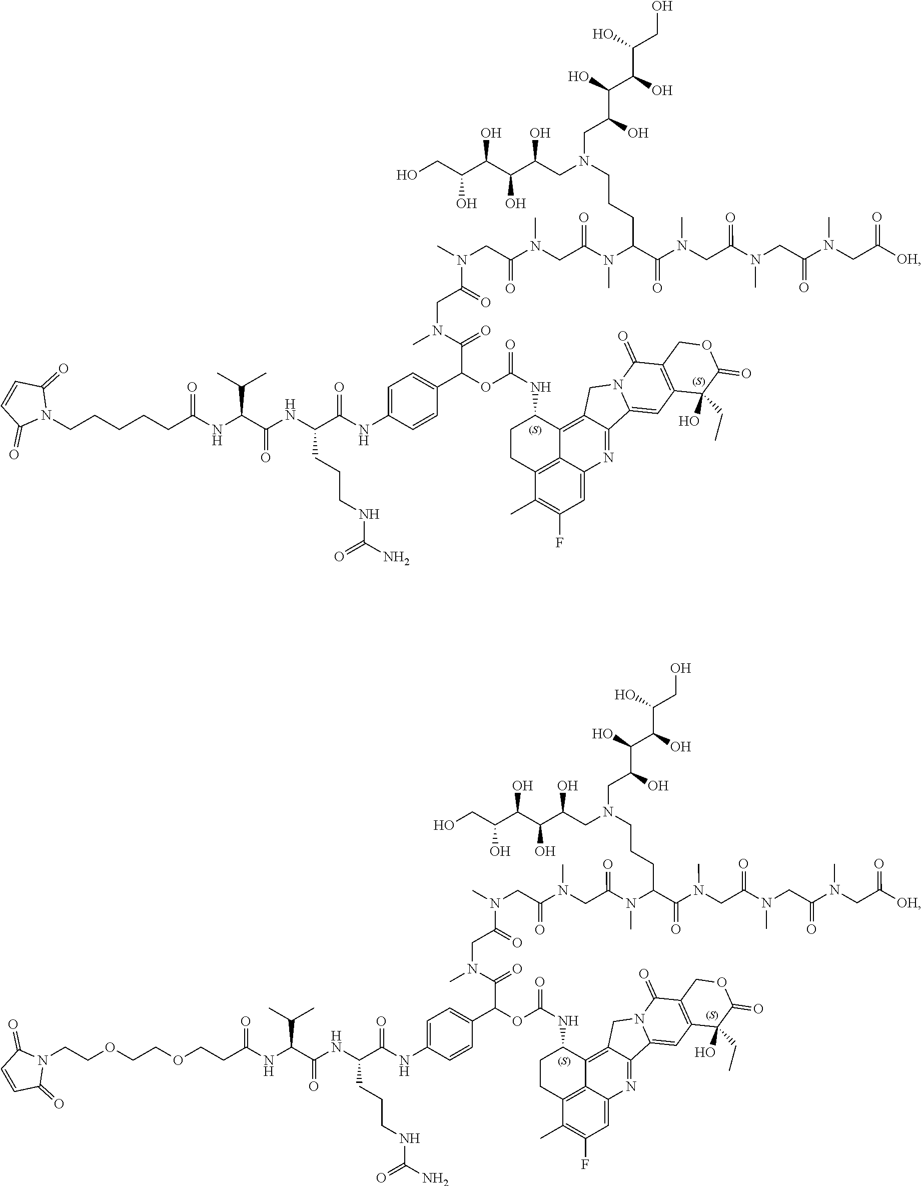

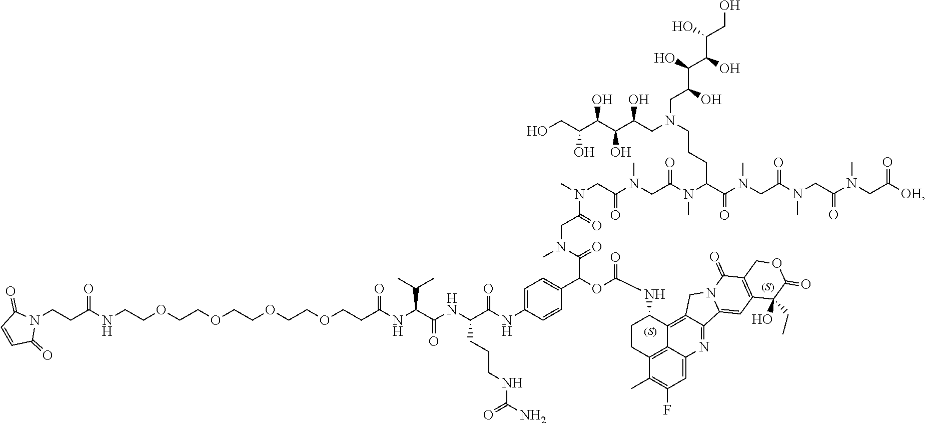

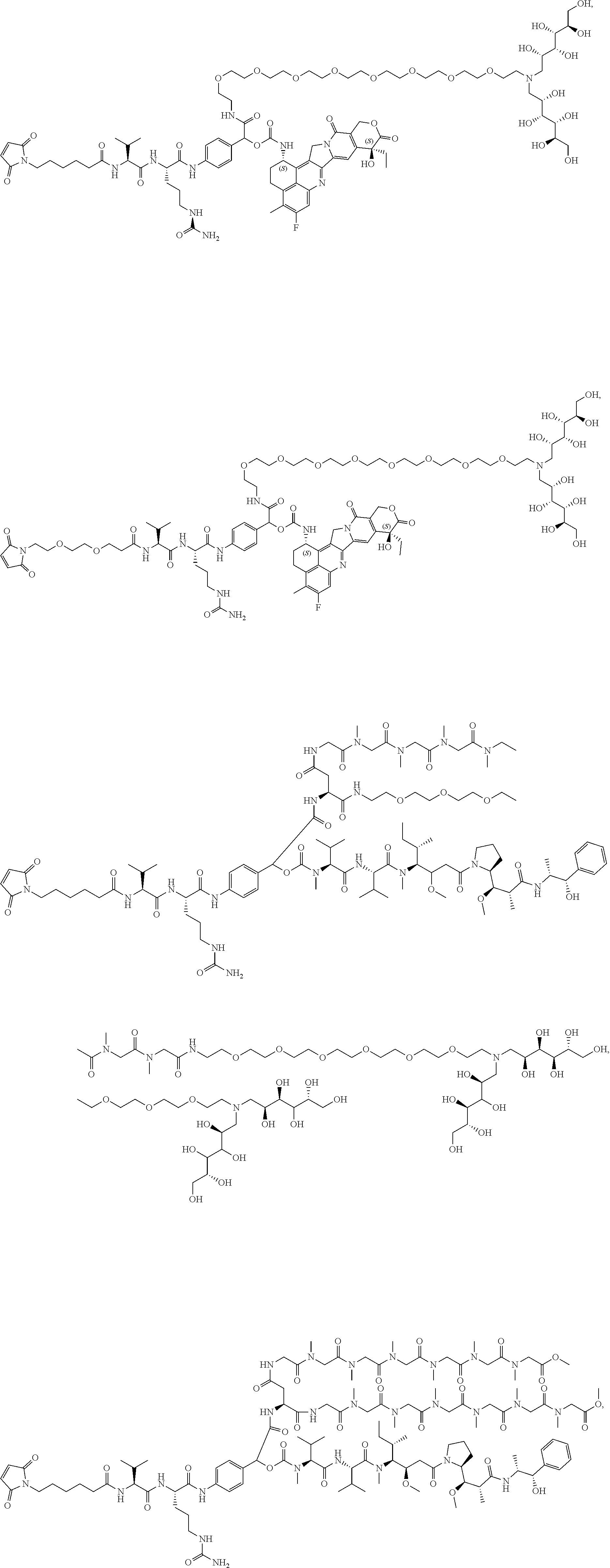

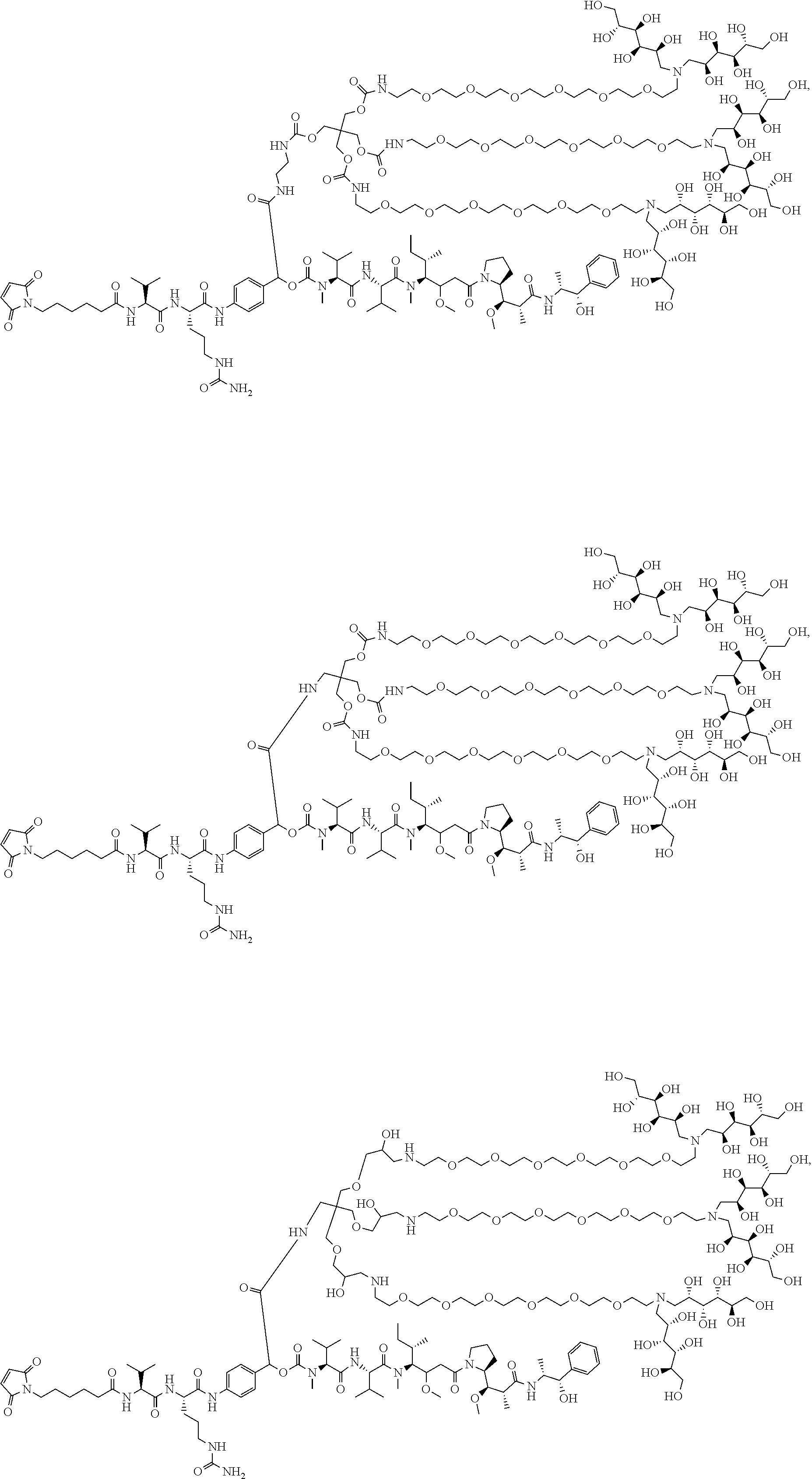

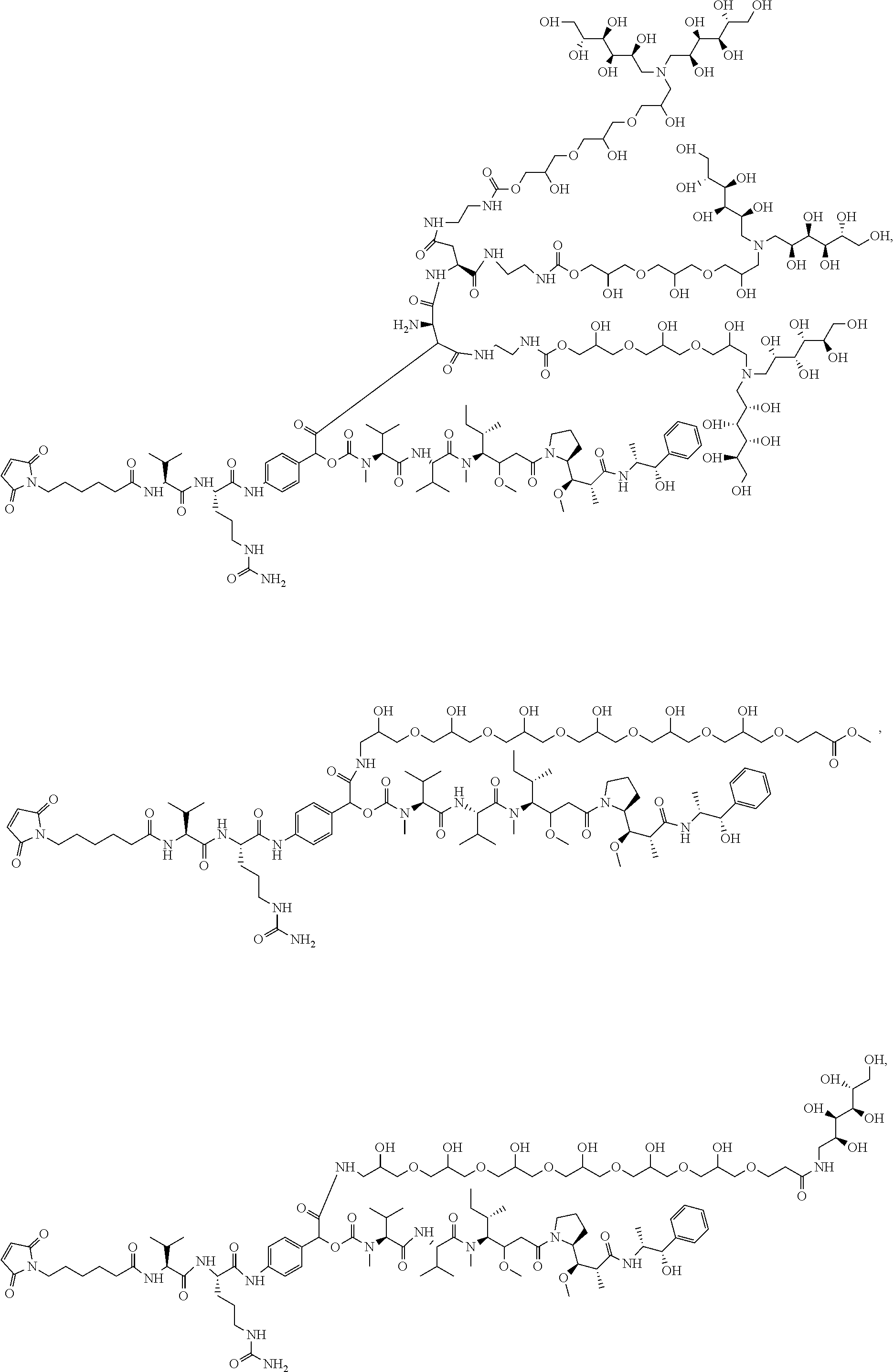

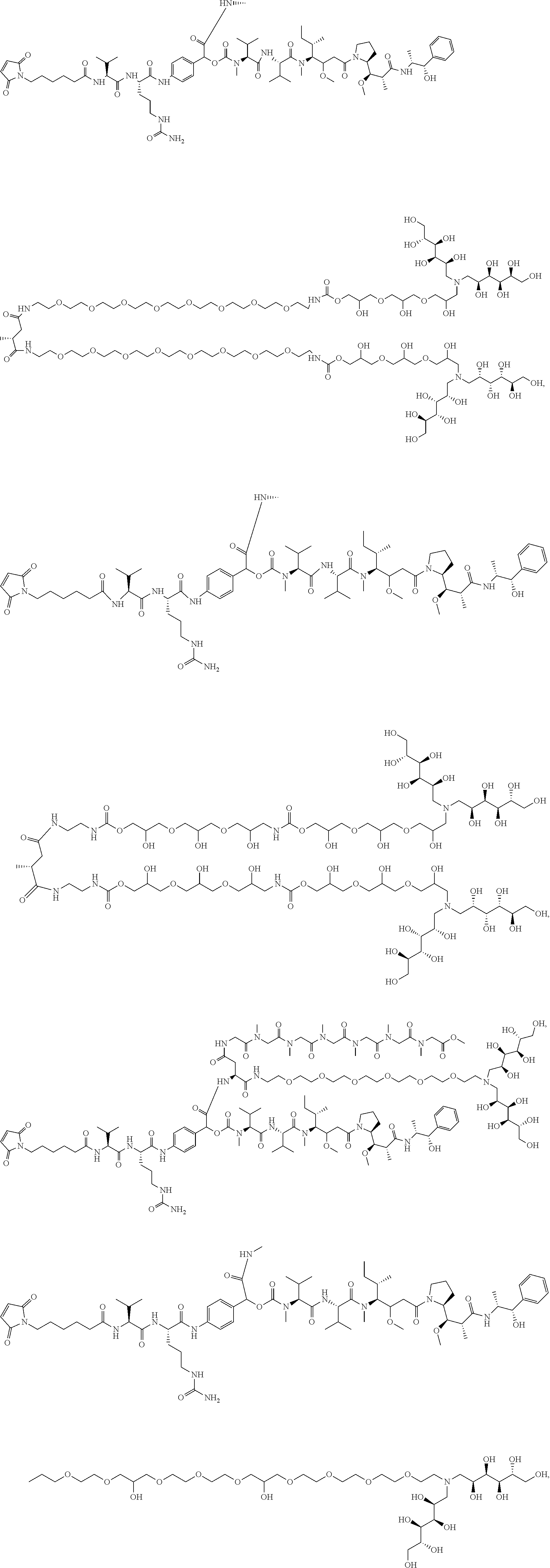

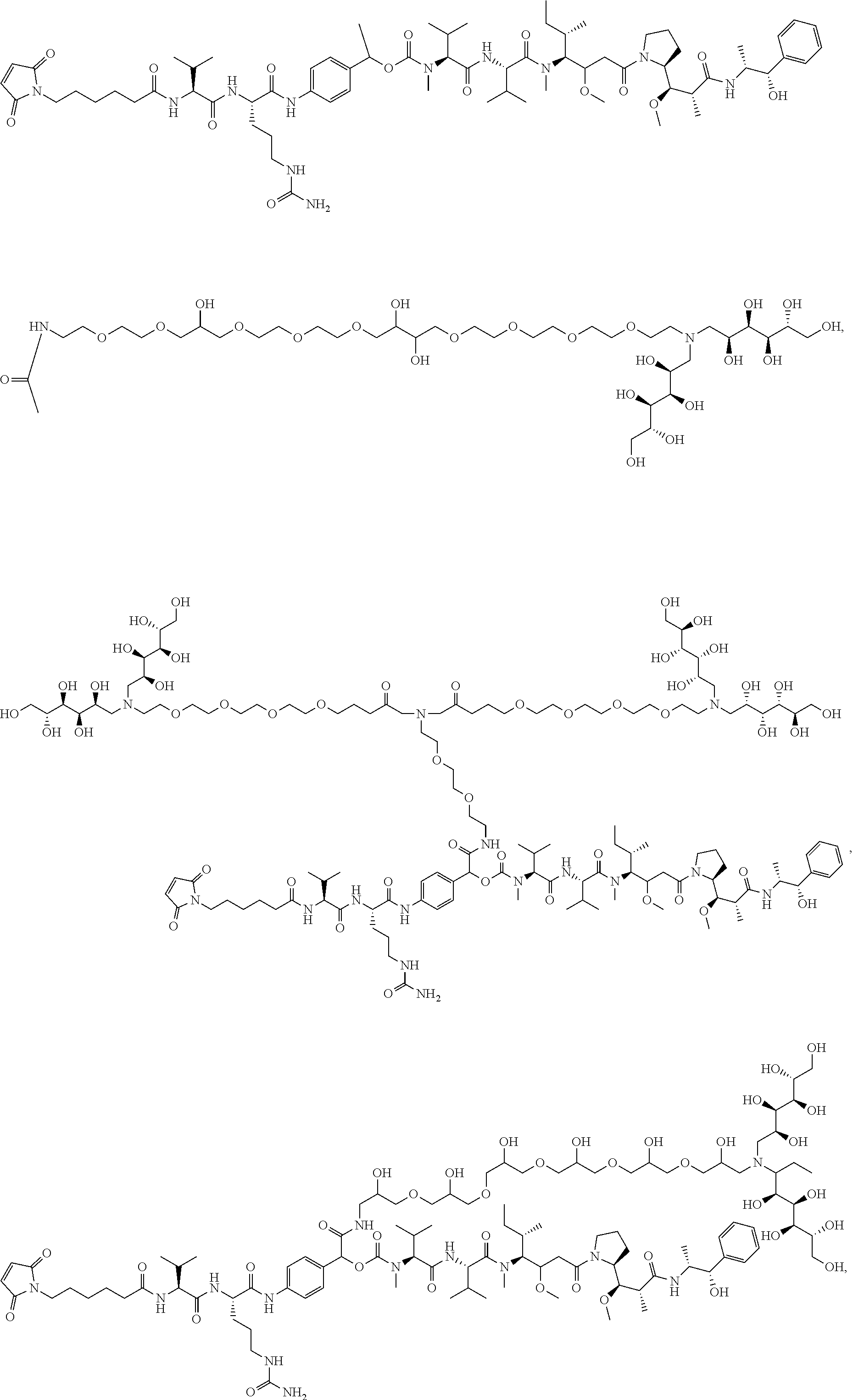

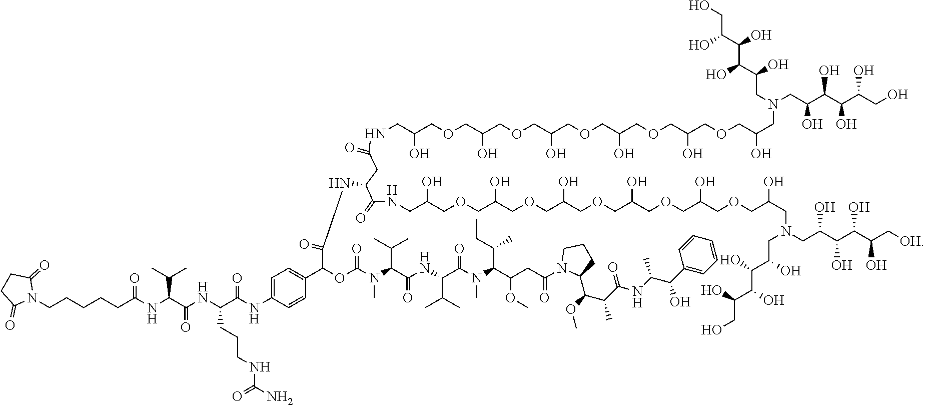 [SMILES: CC[C@H](C)[C@@H](C(CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)CCC2=O)C(C)C)cc1)C(C)C]

Embodiment 65. The conjugate of any of Embodiment 64, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

Embodiment 66. The conjugate of any of Embodiments 64-65, selected from the following:

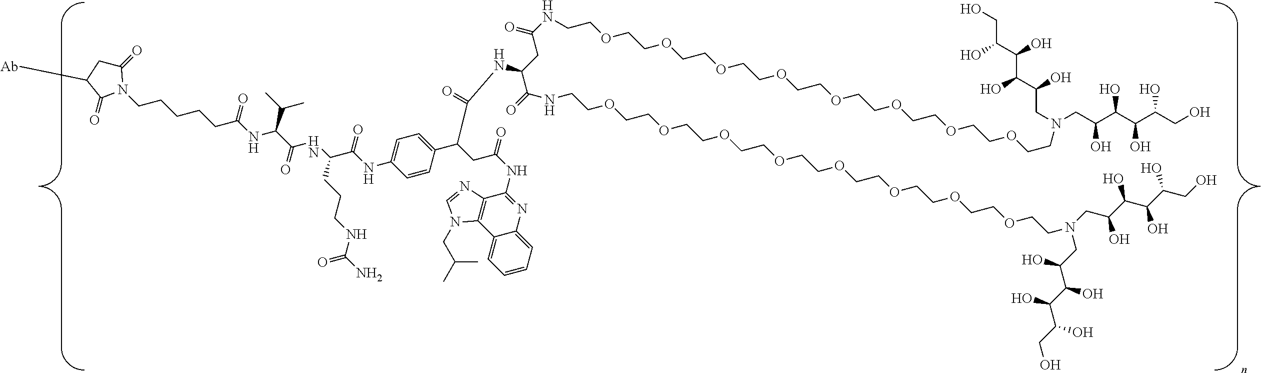

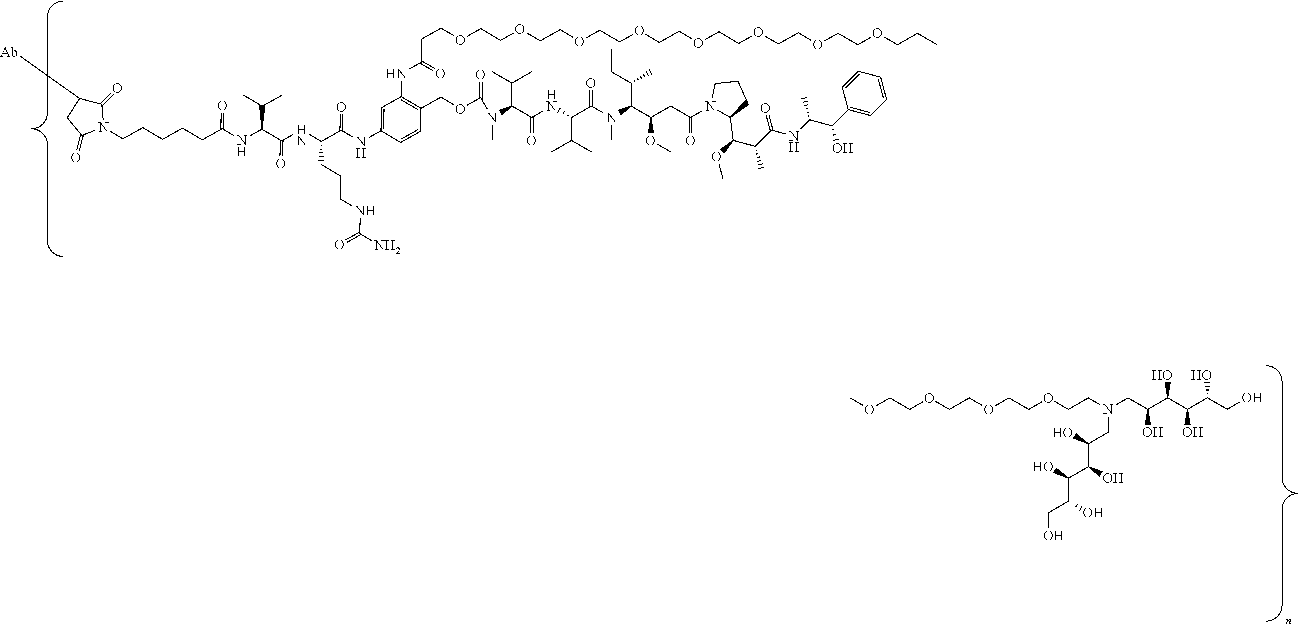

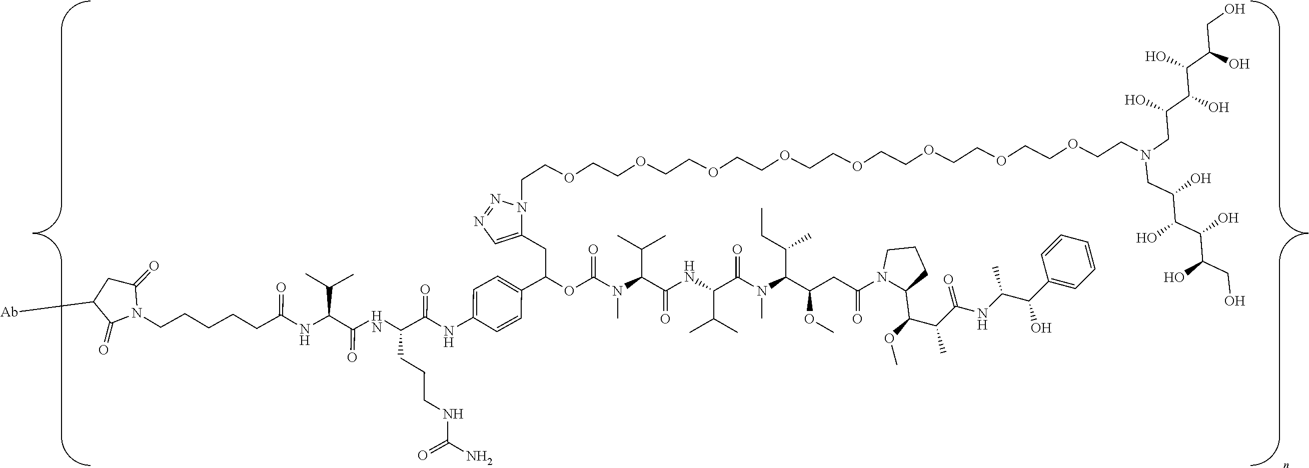

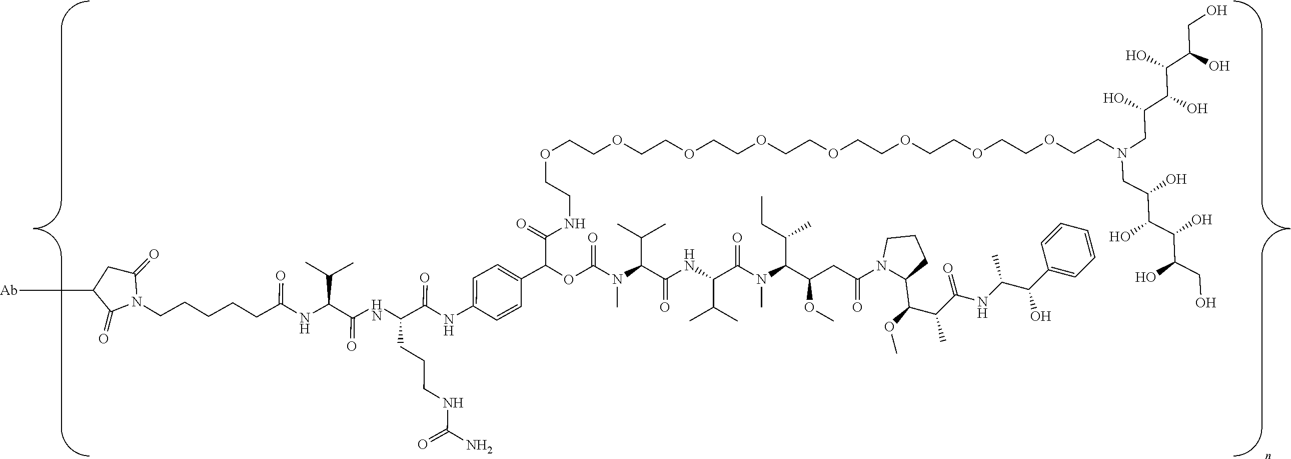

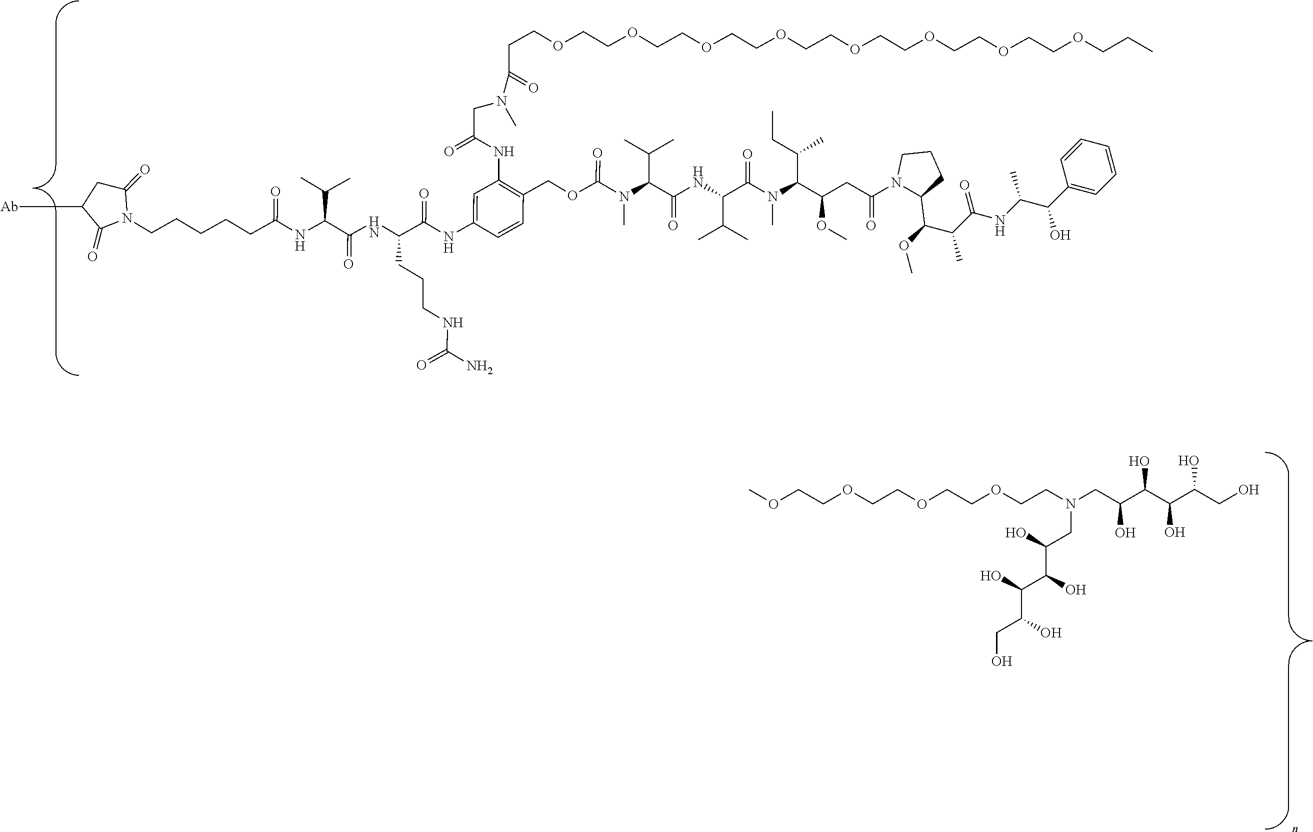

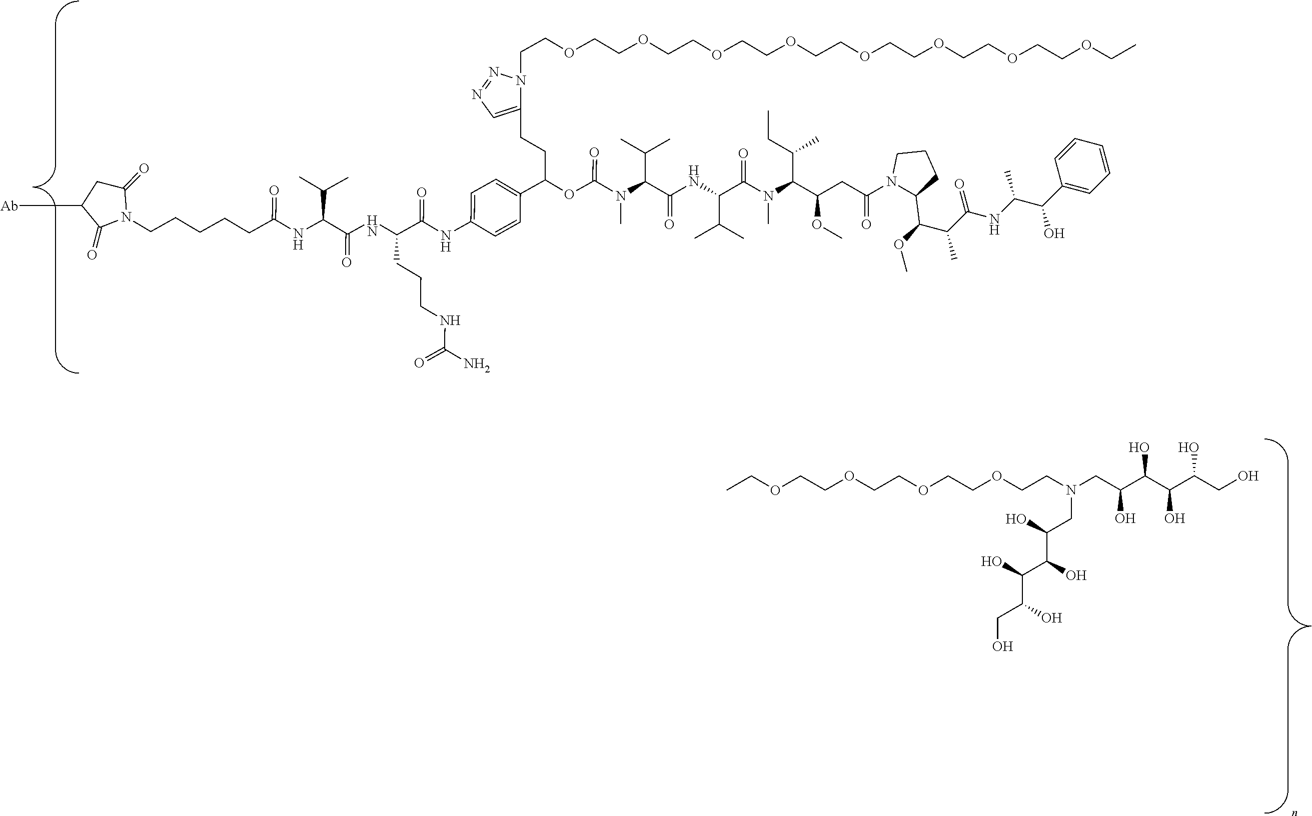

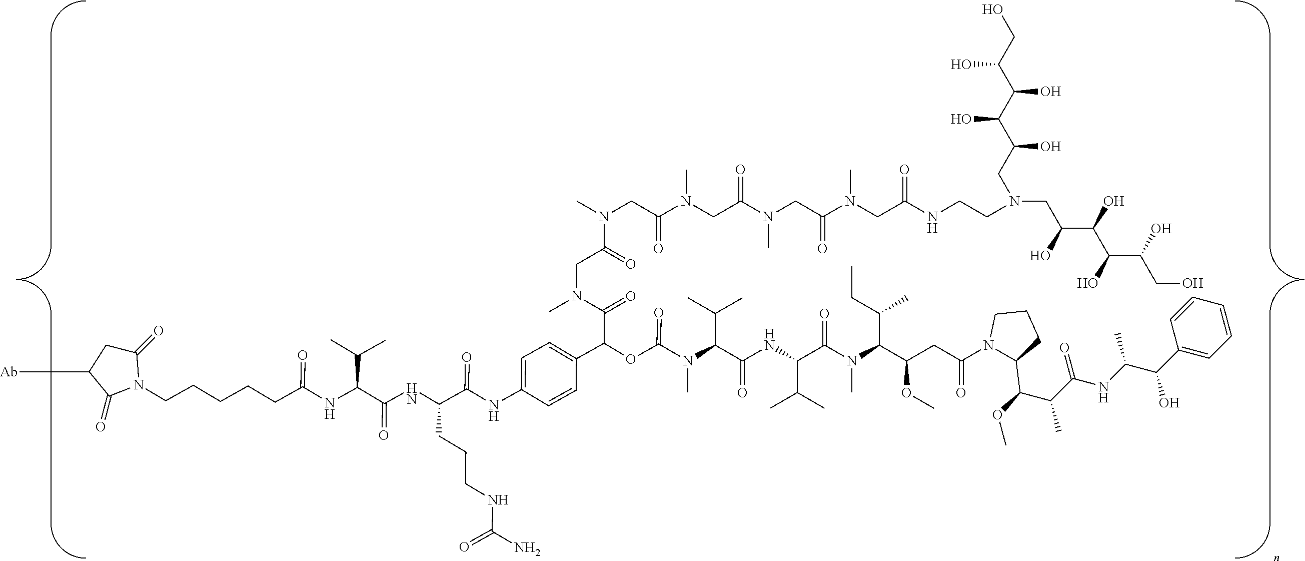

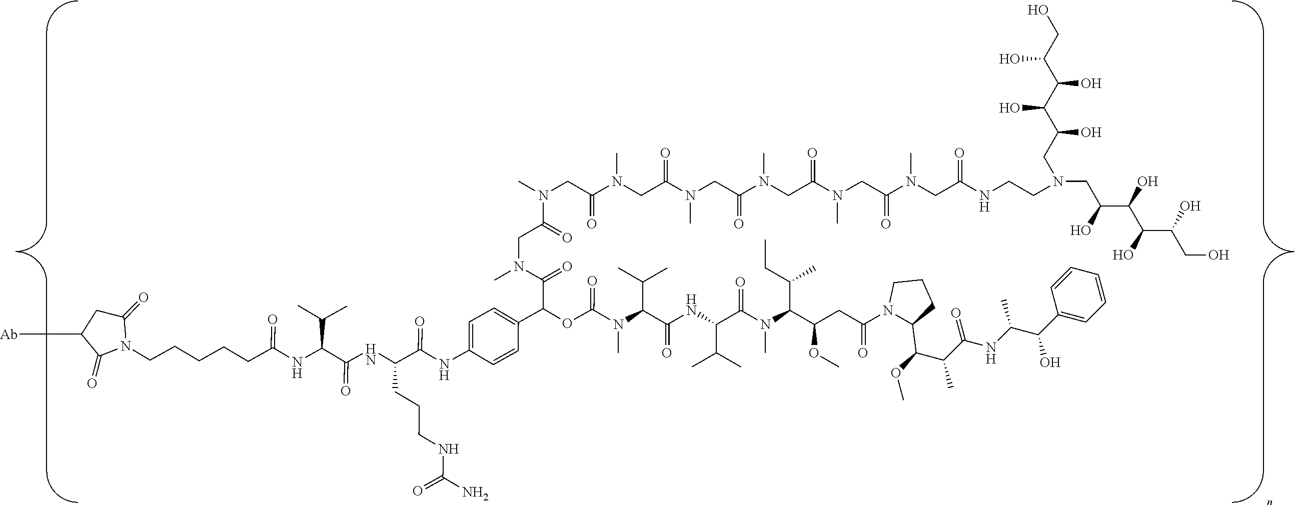

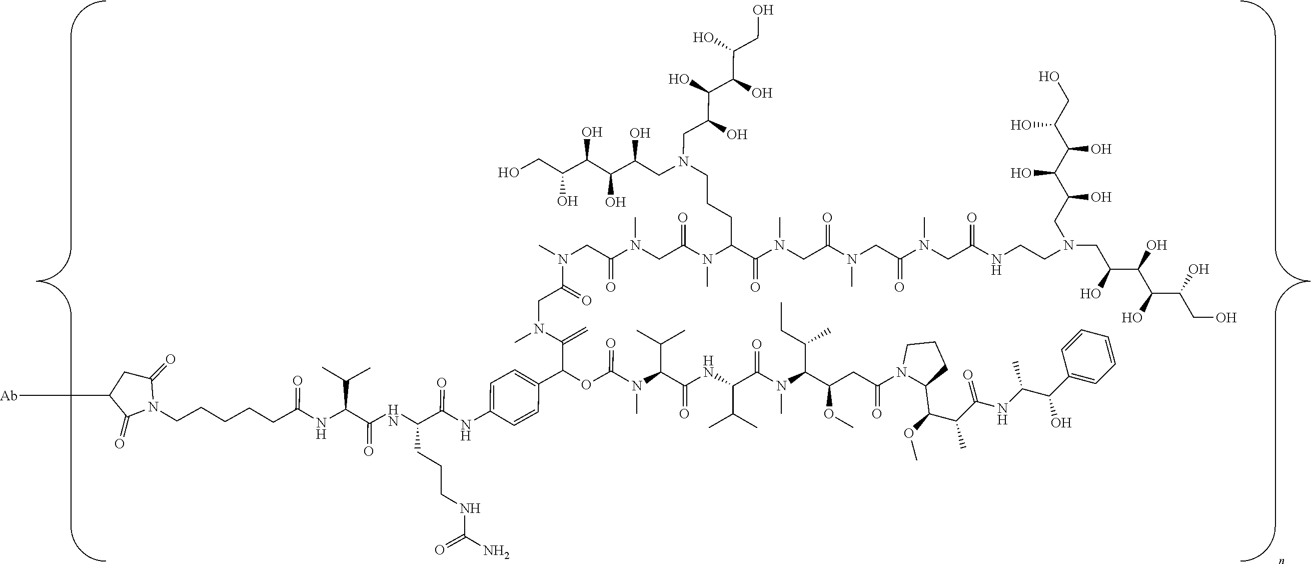

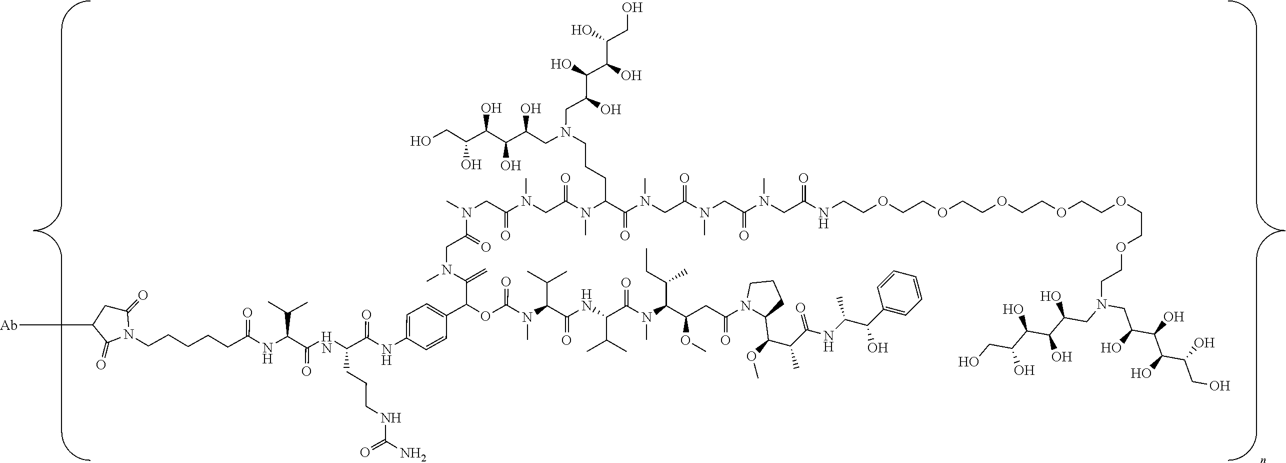

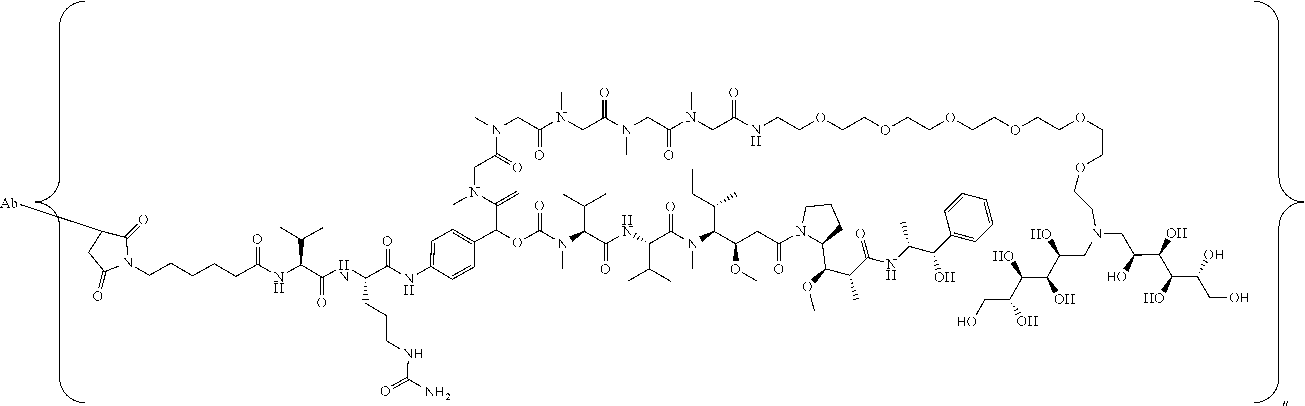

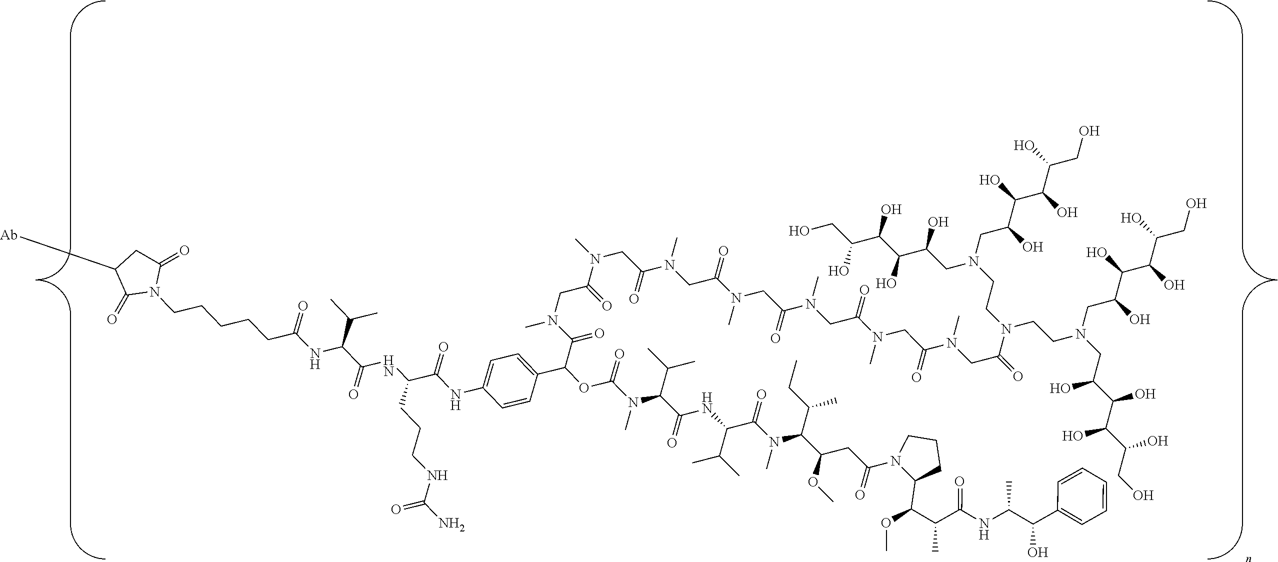

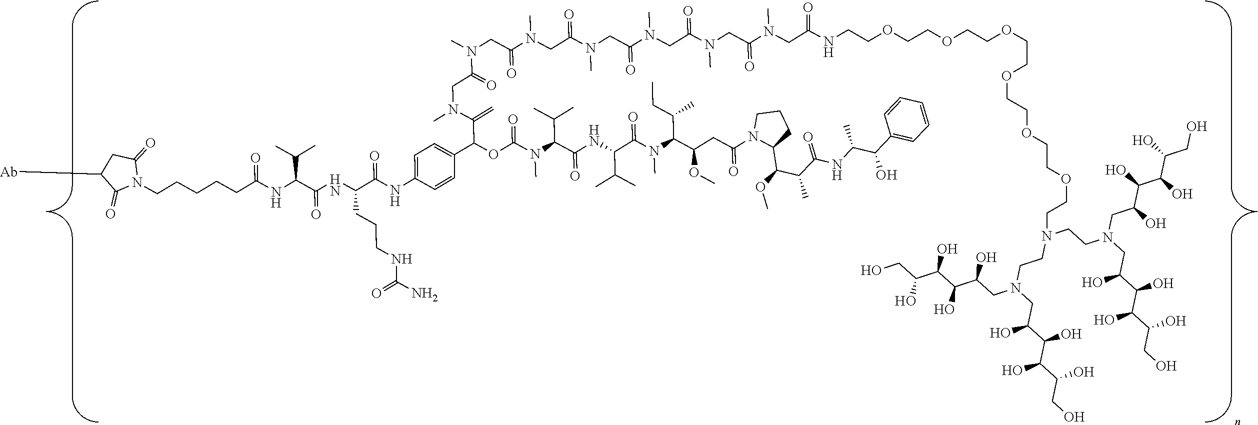

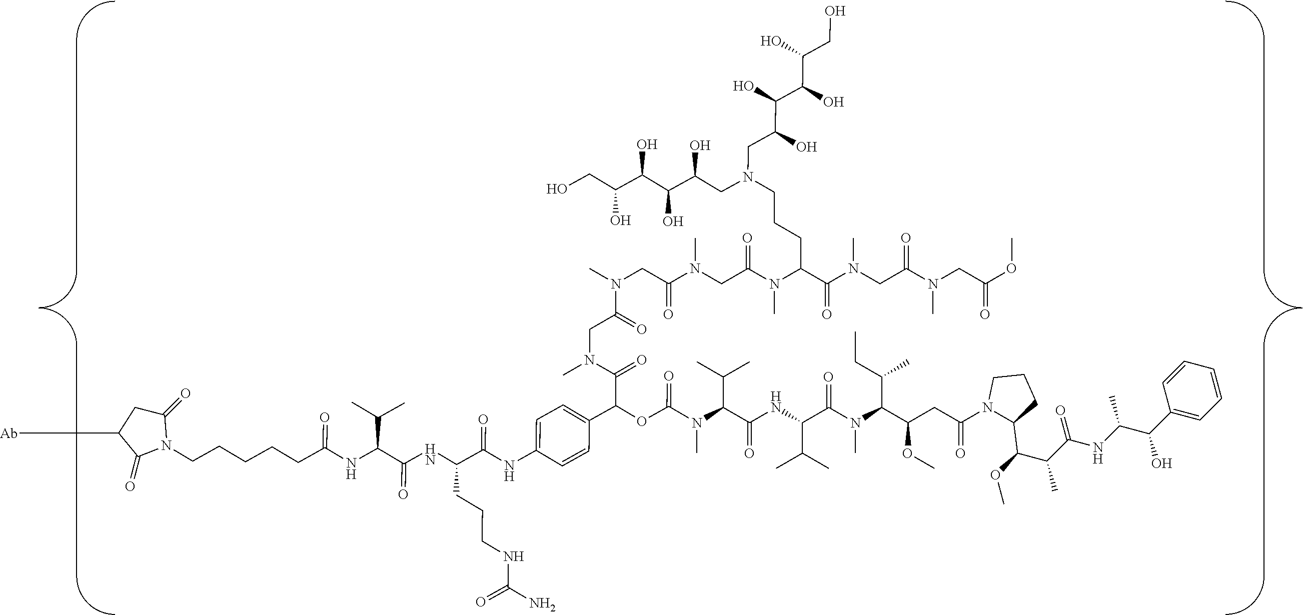

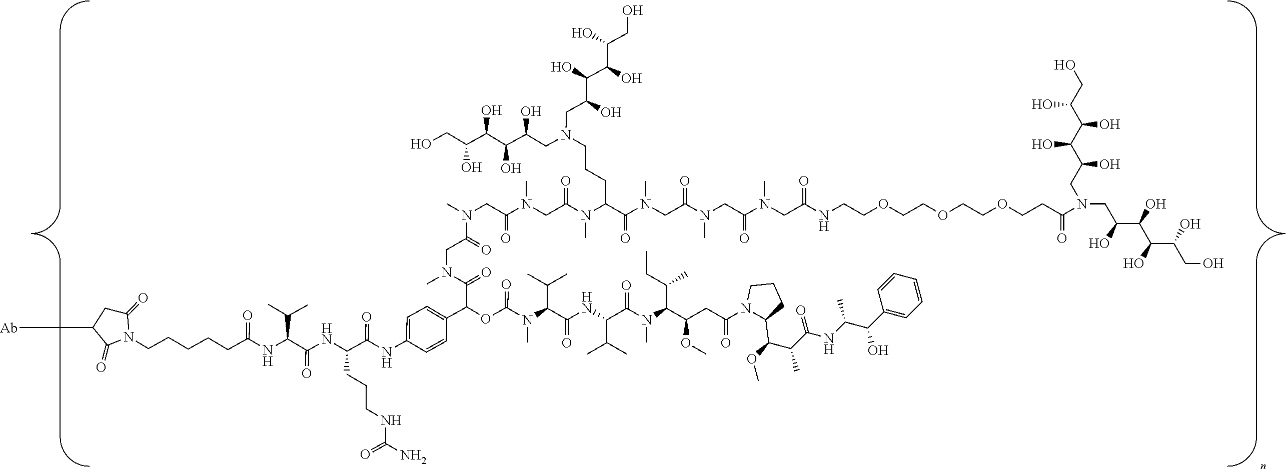

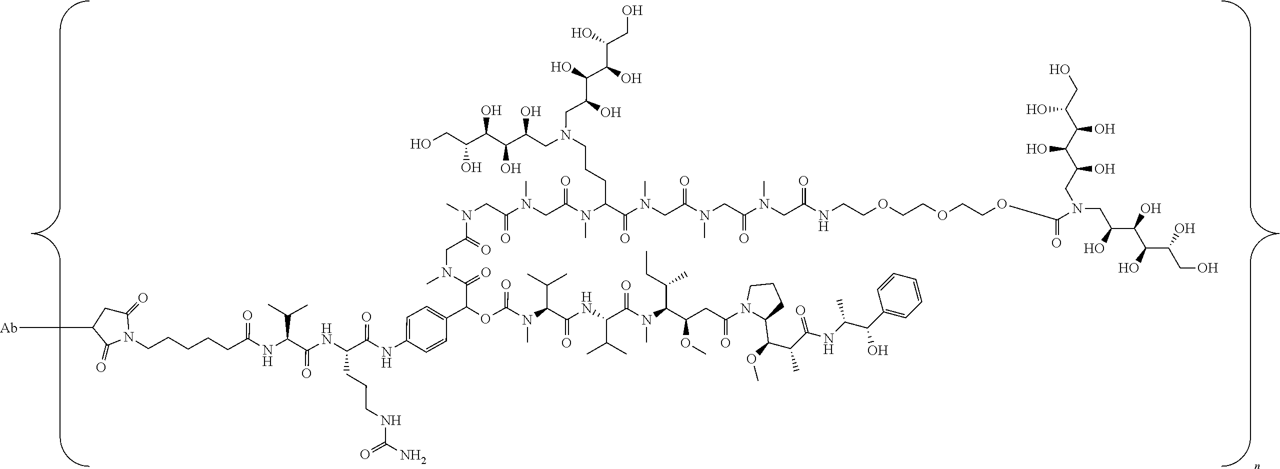

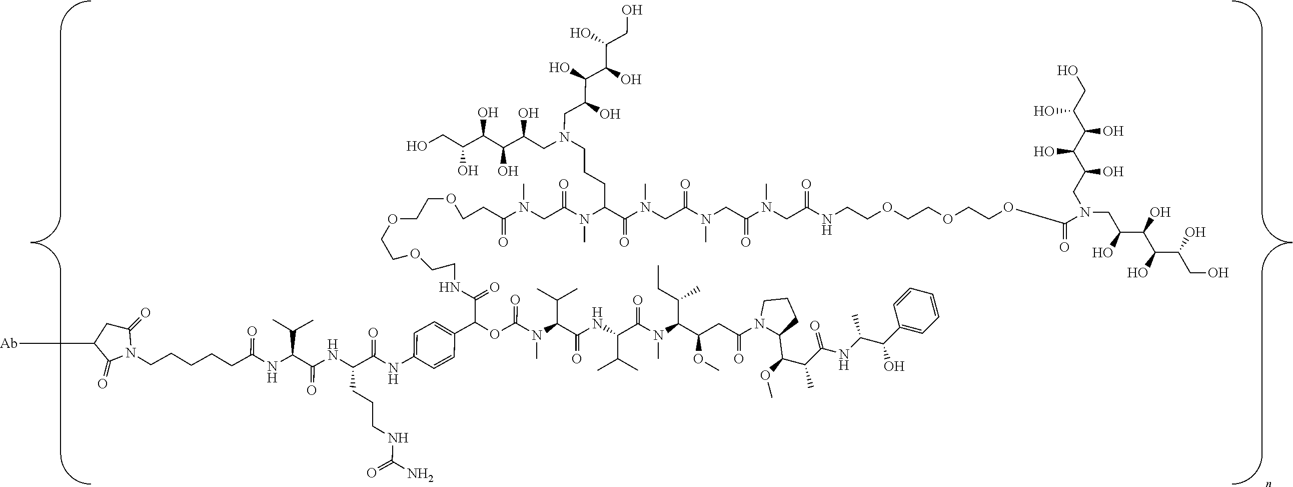

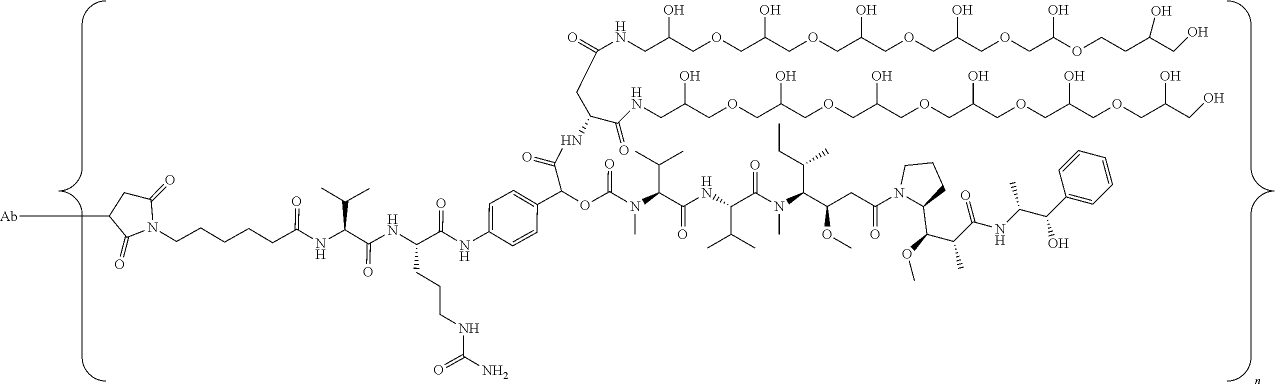

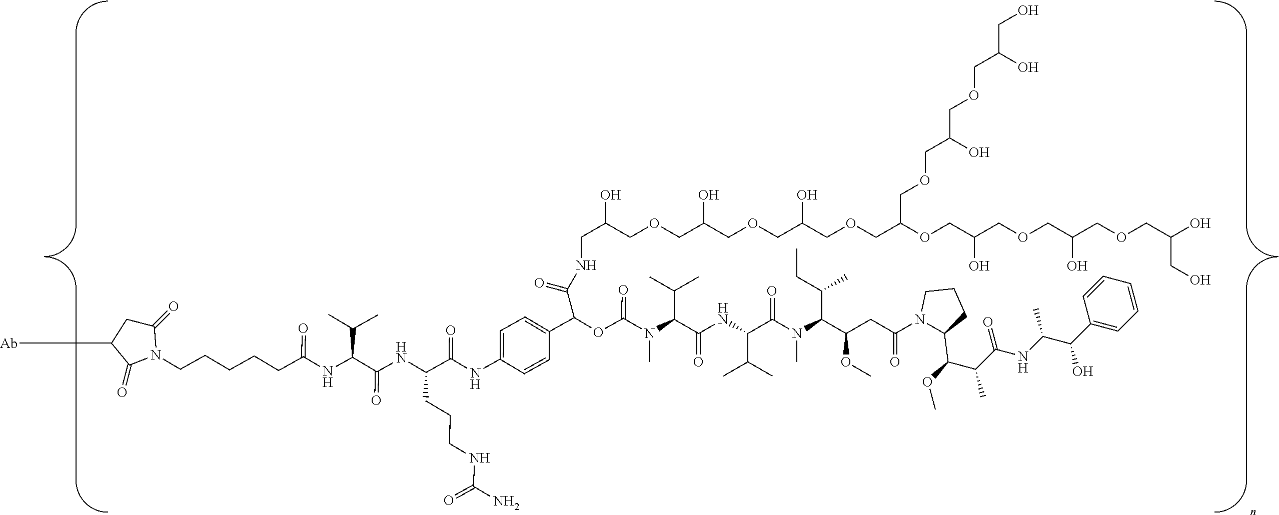

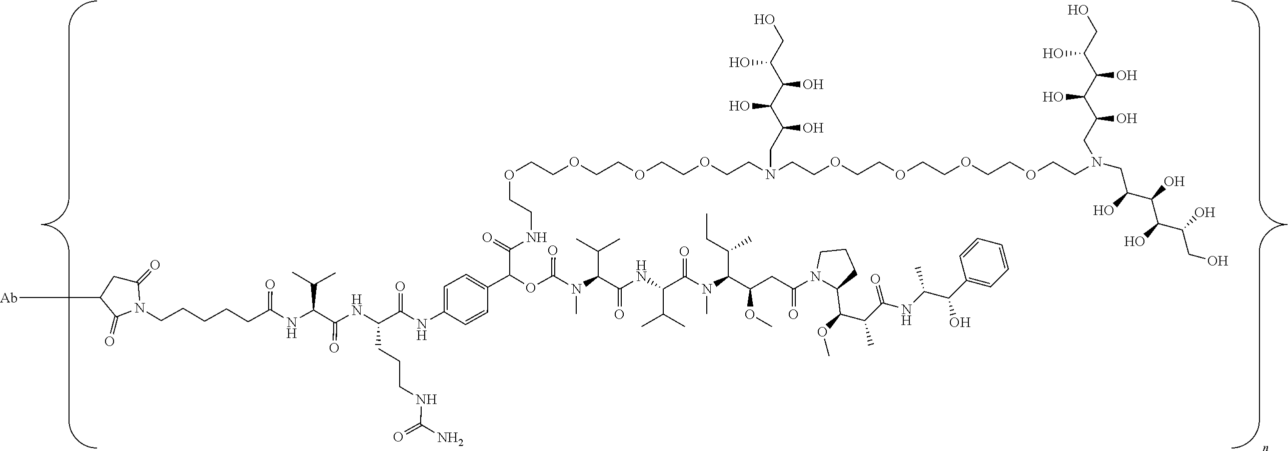

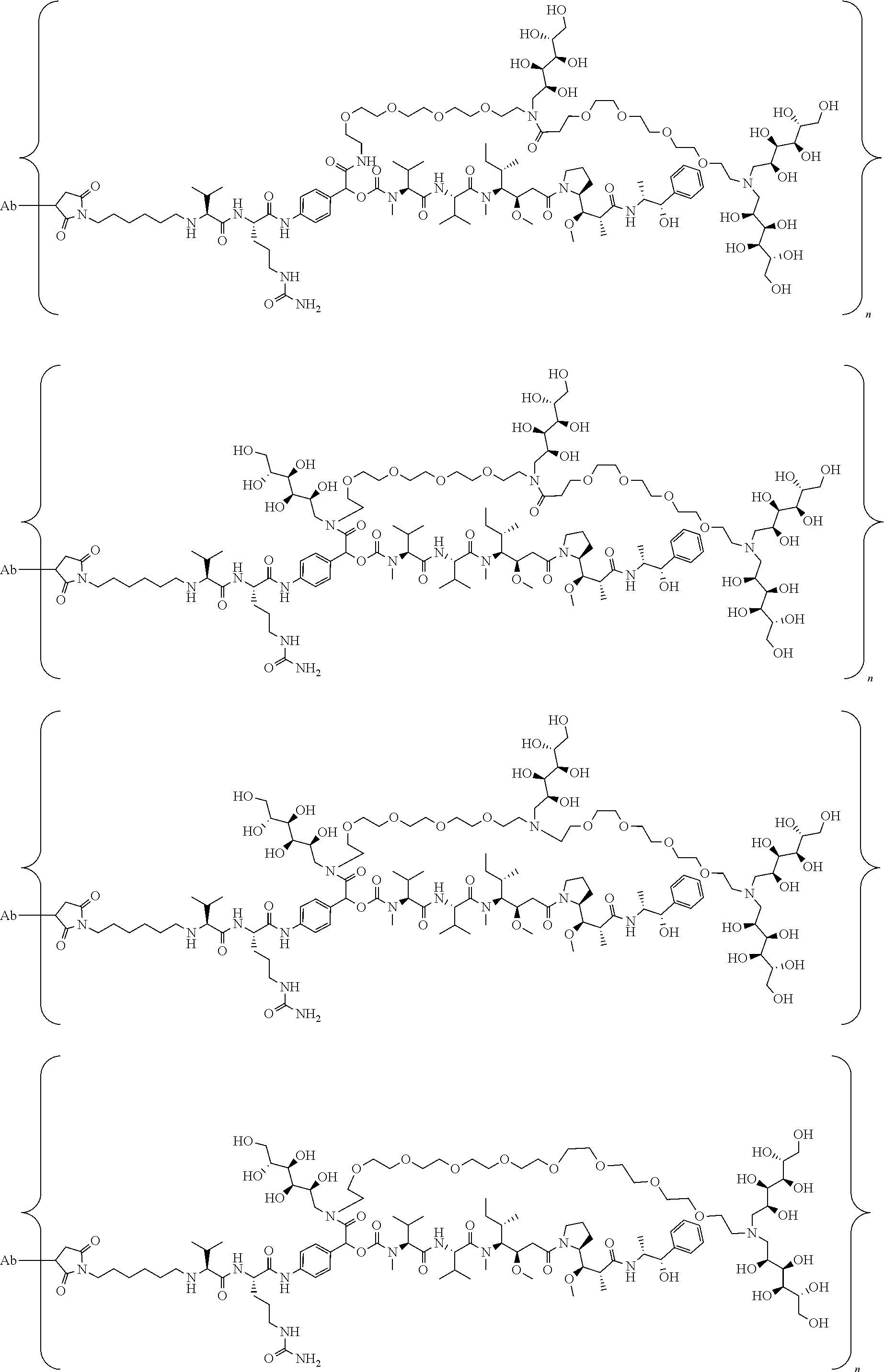

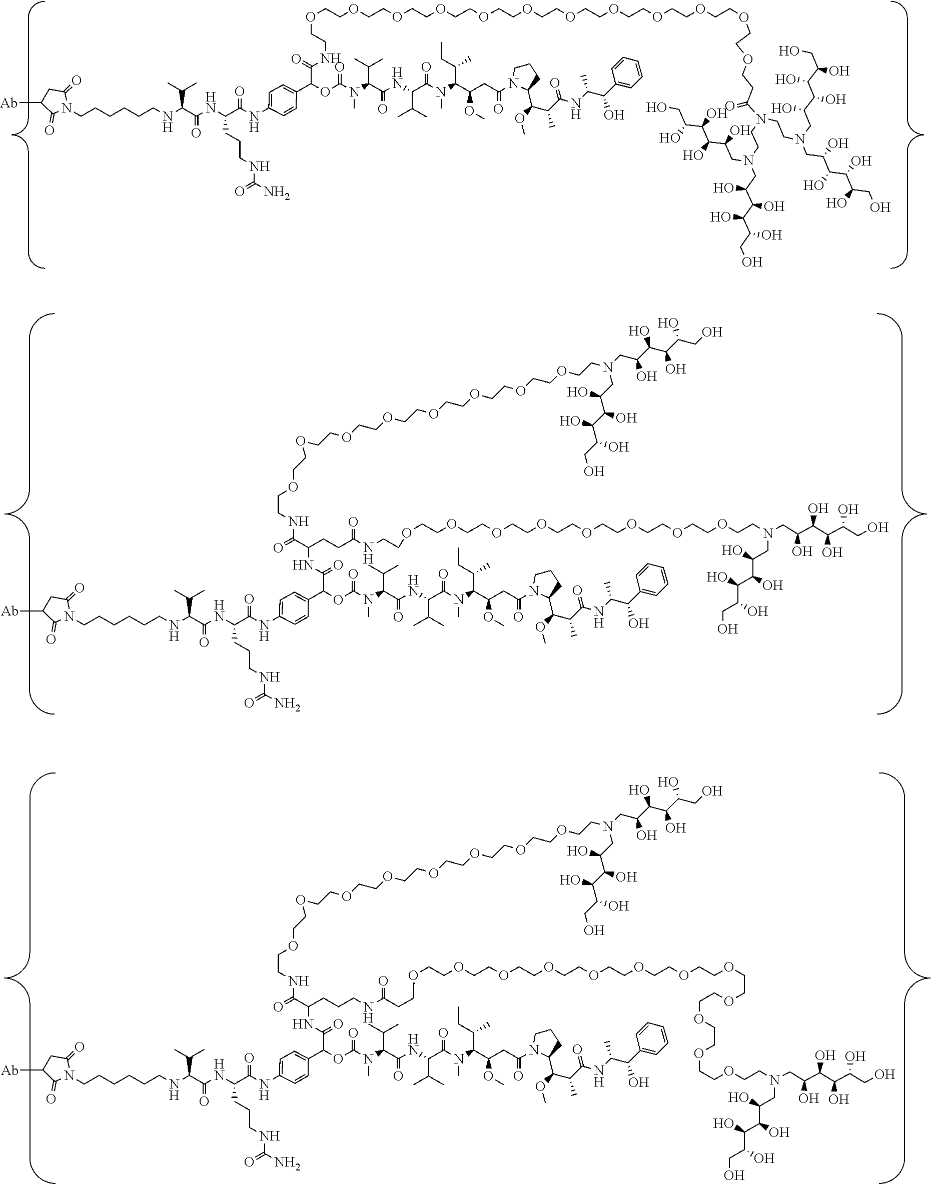

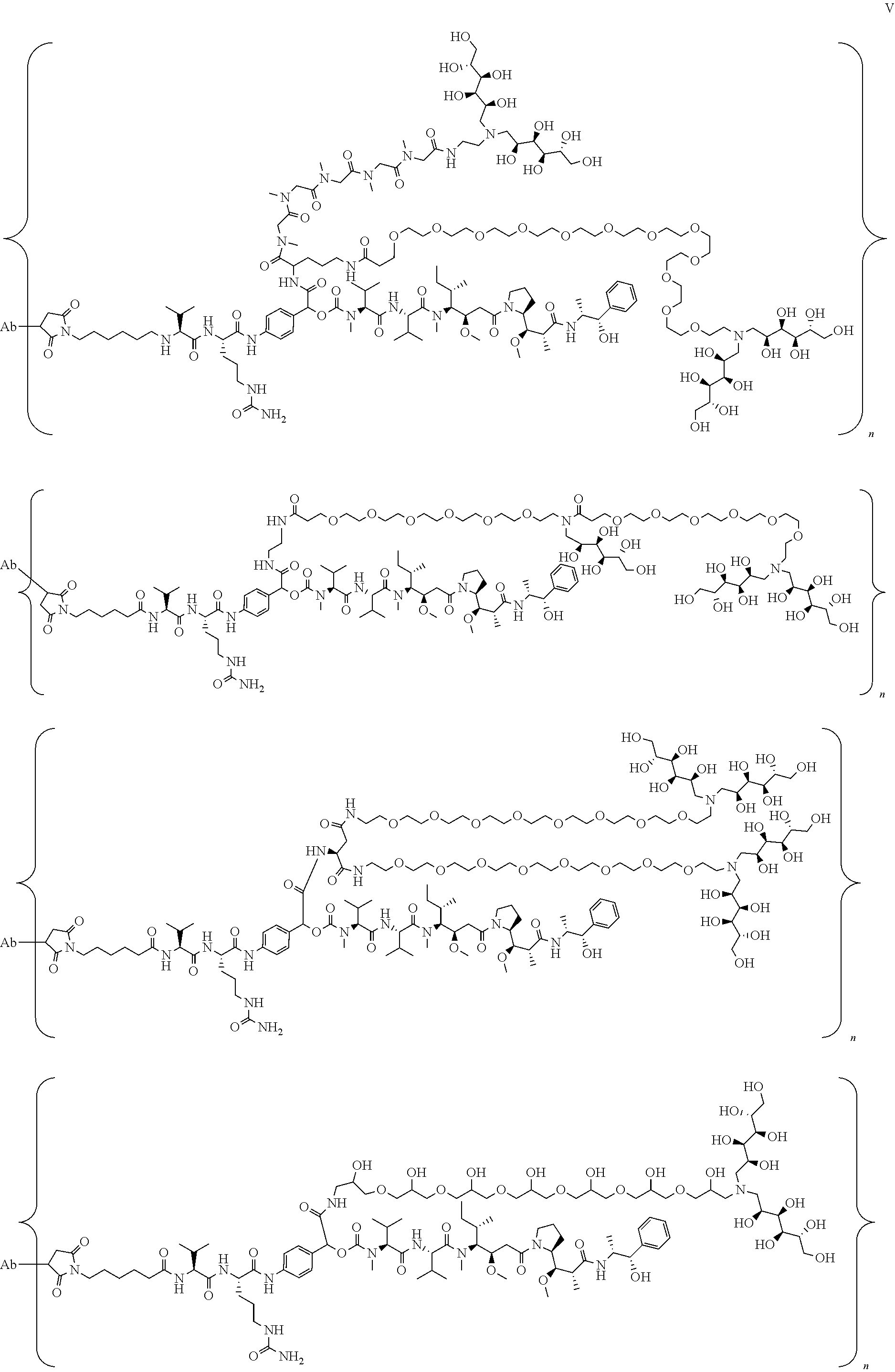

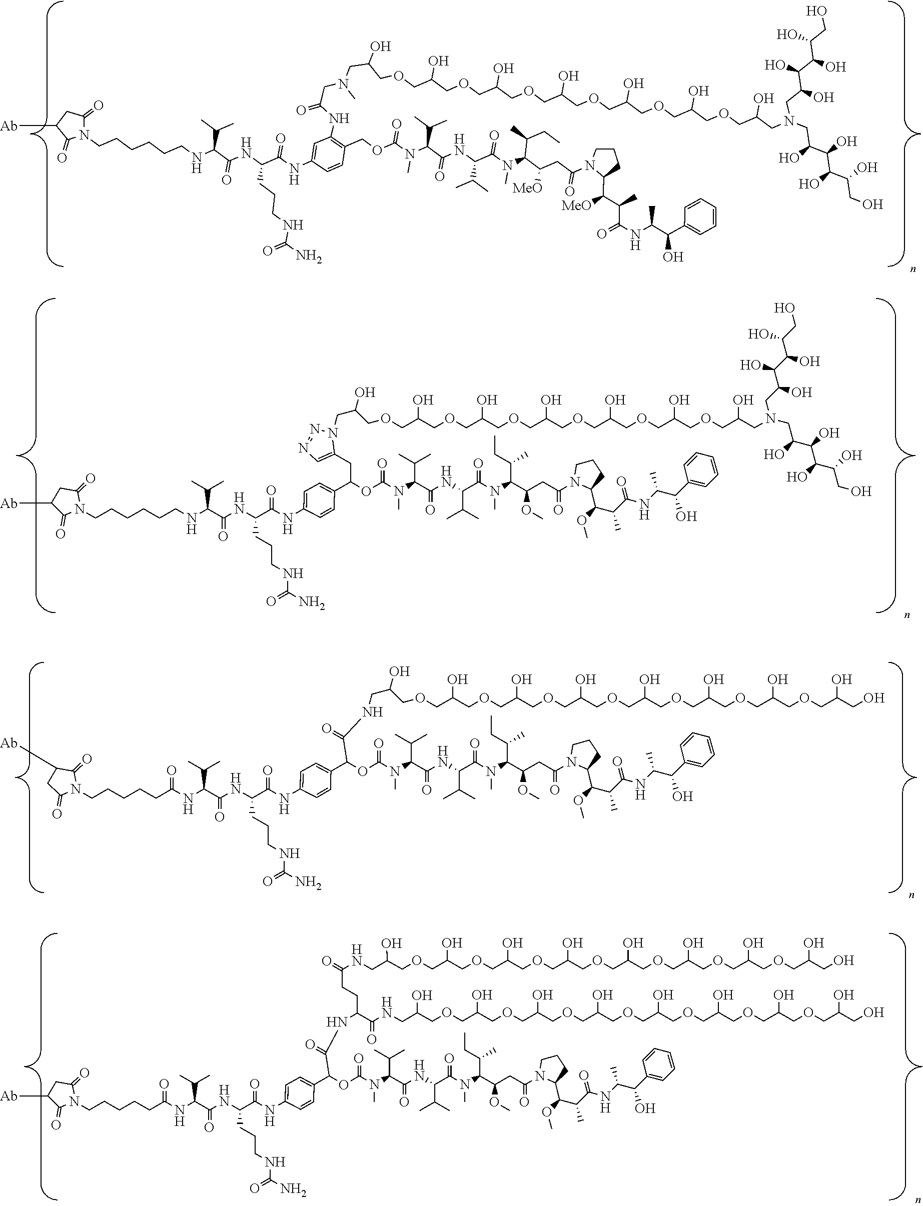

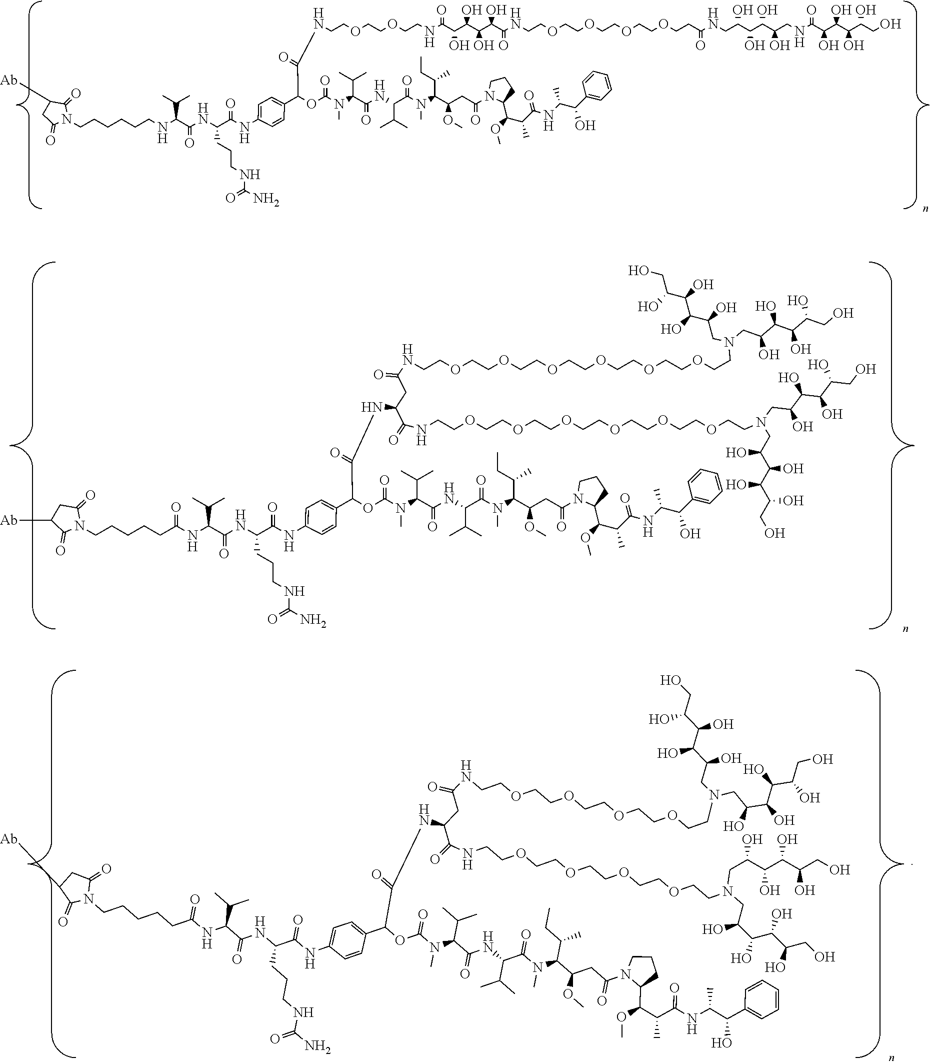

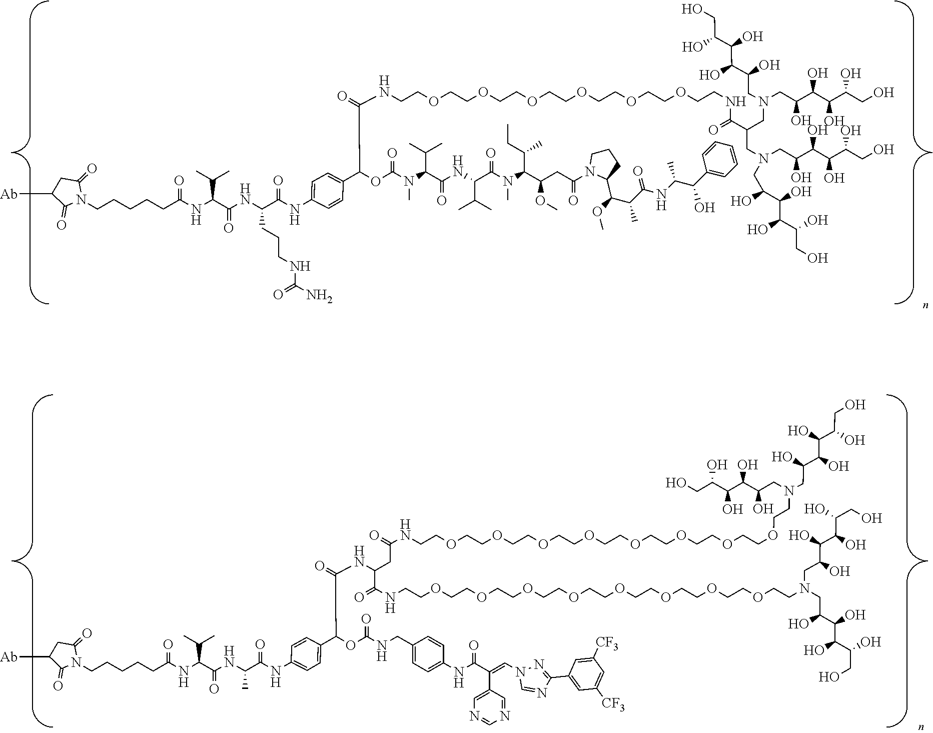

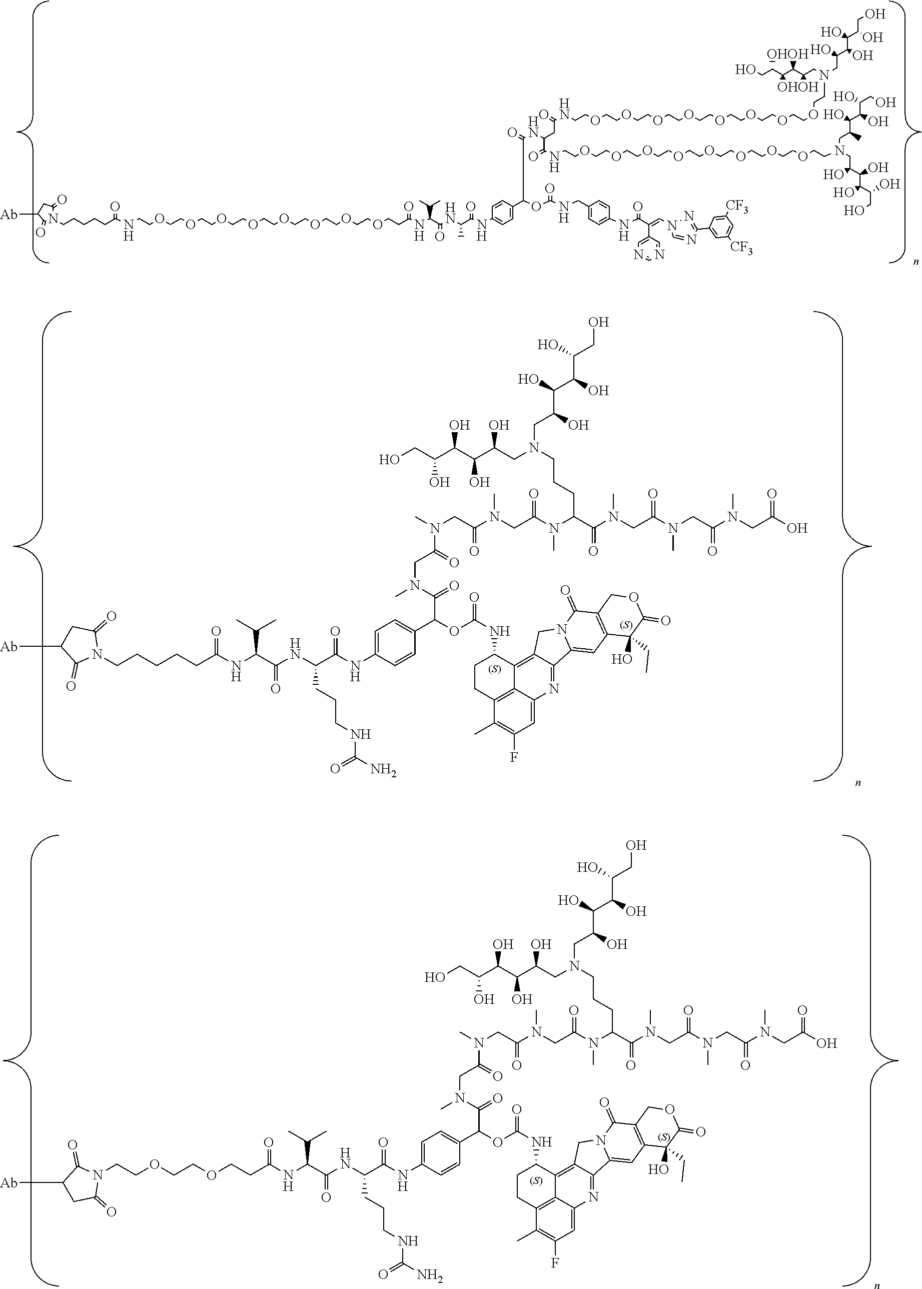

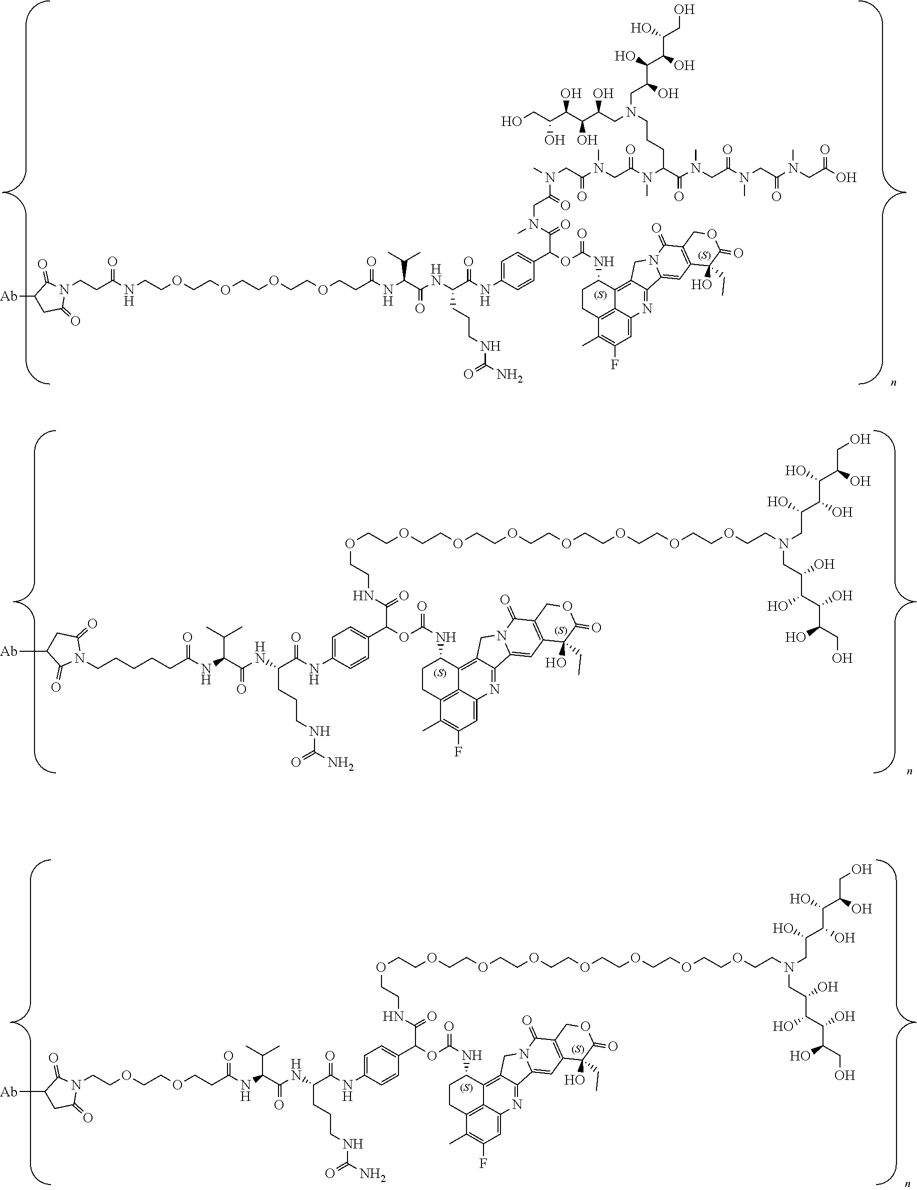

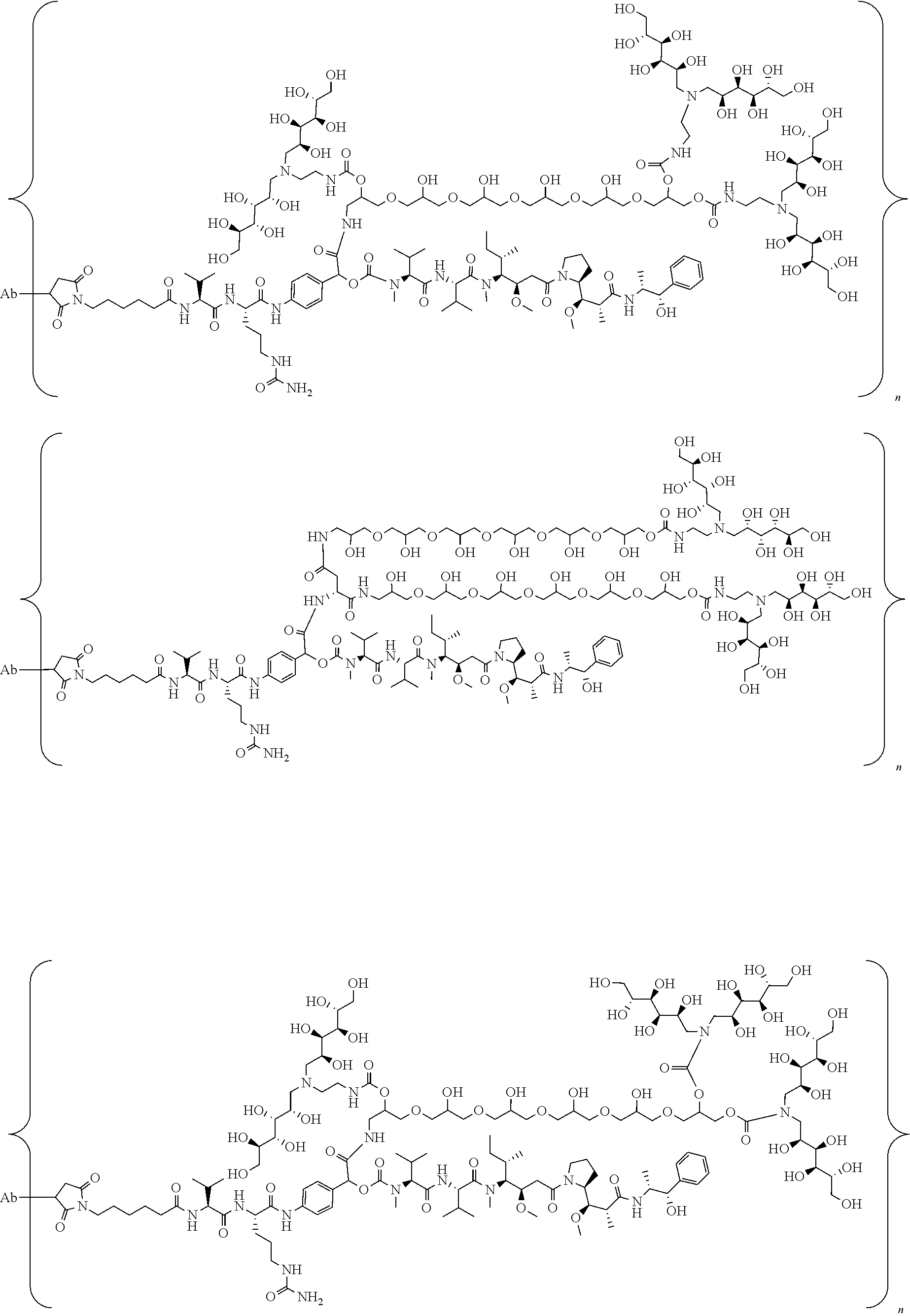

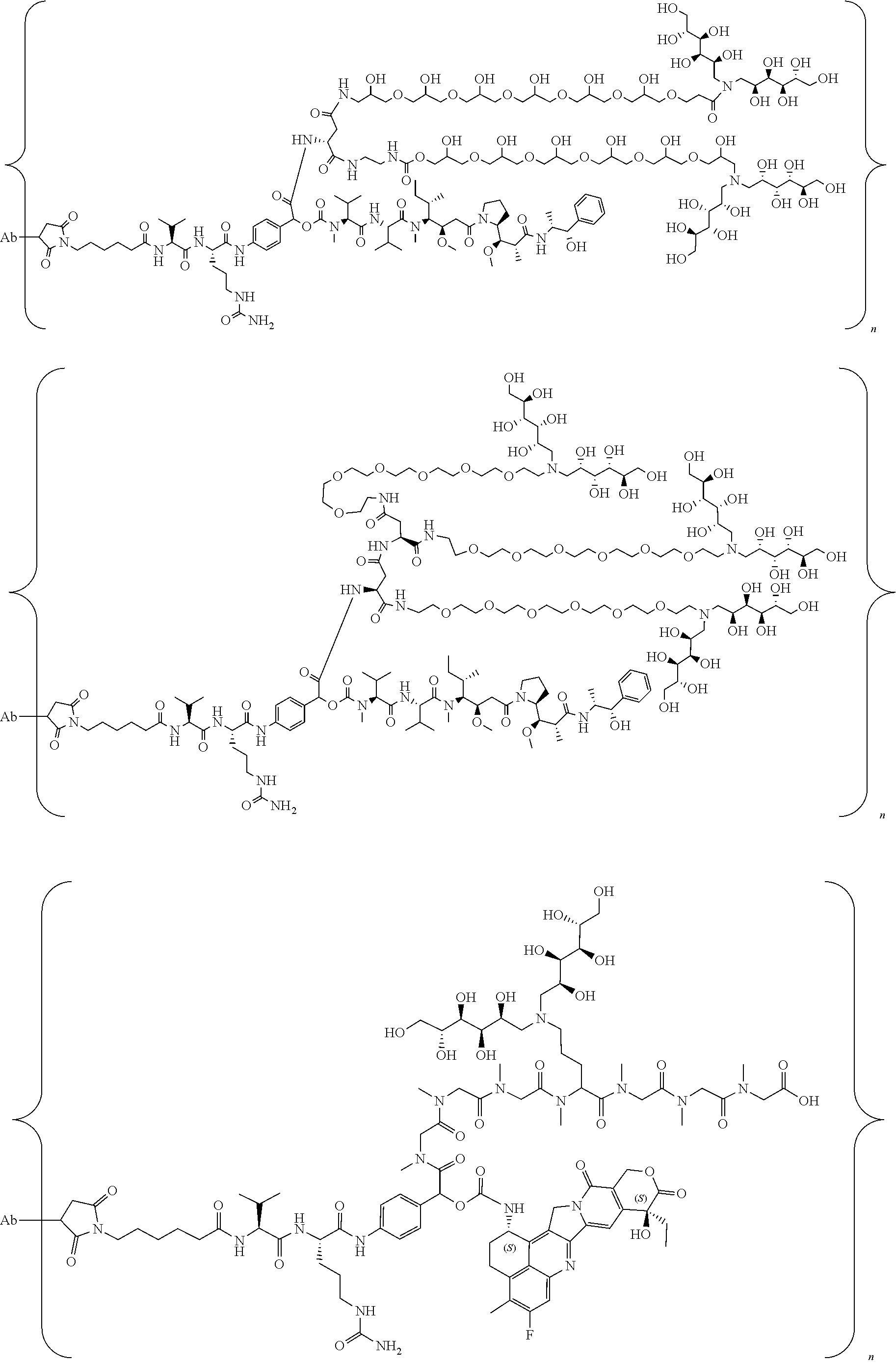

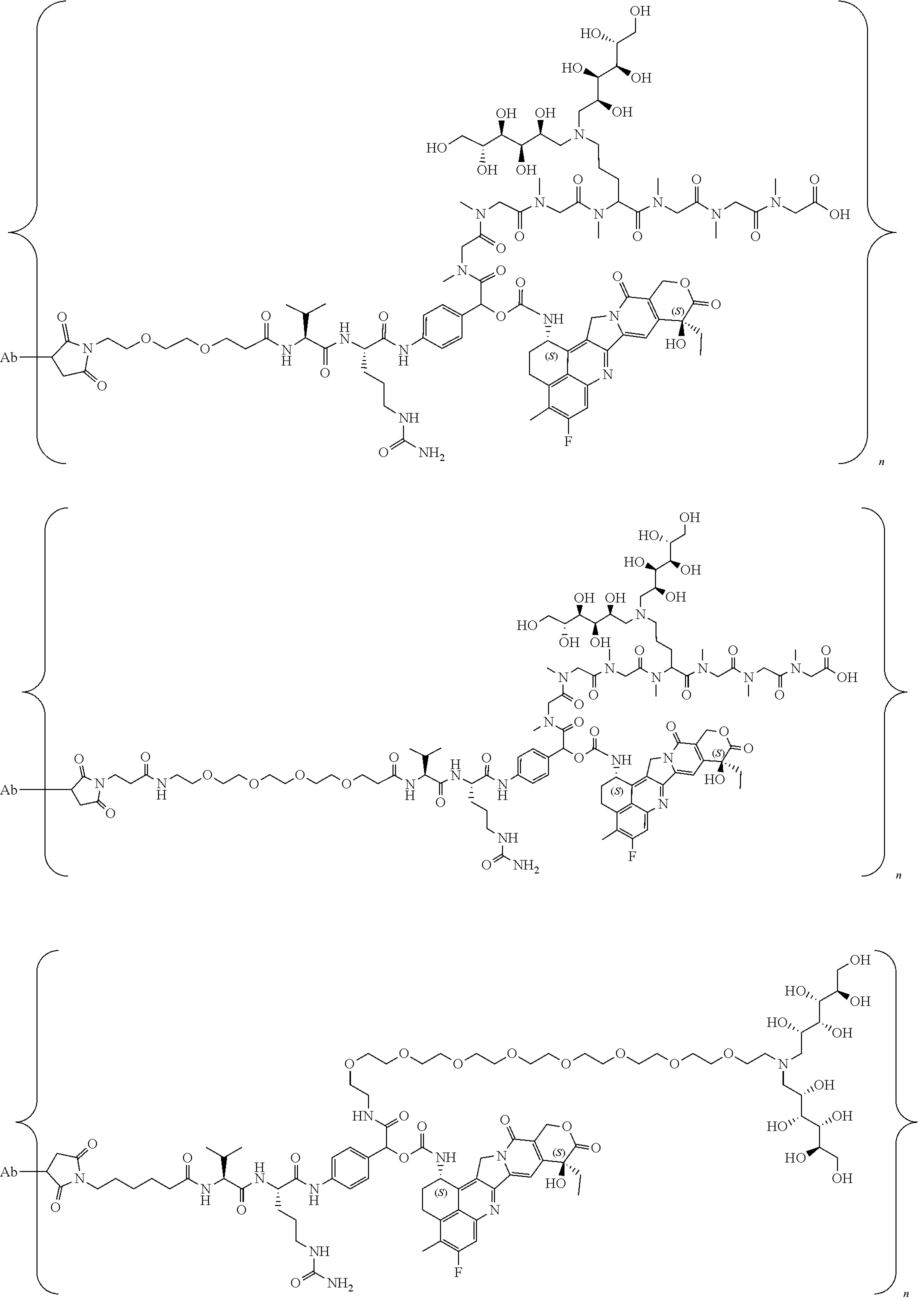

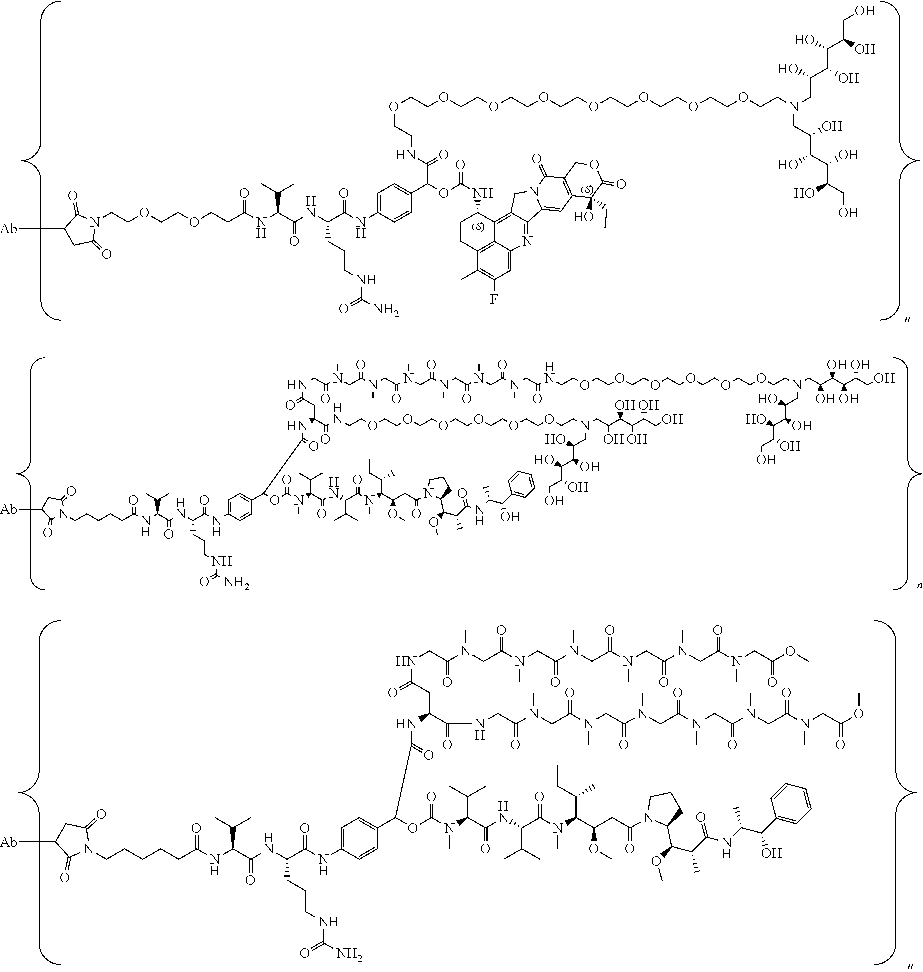

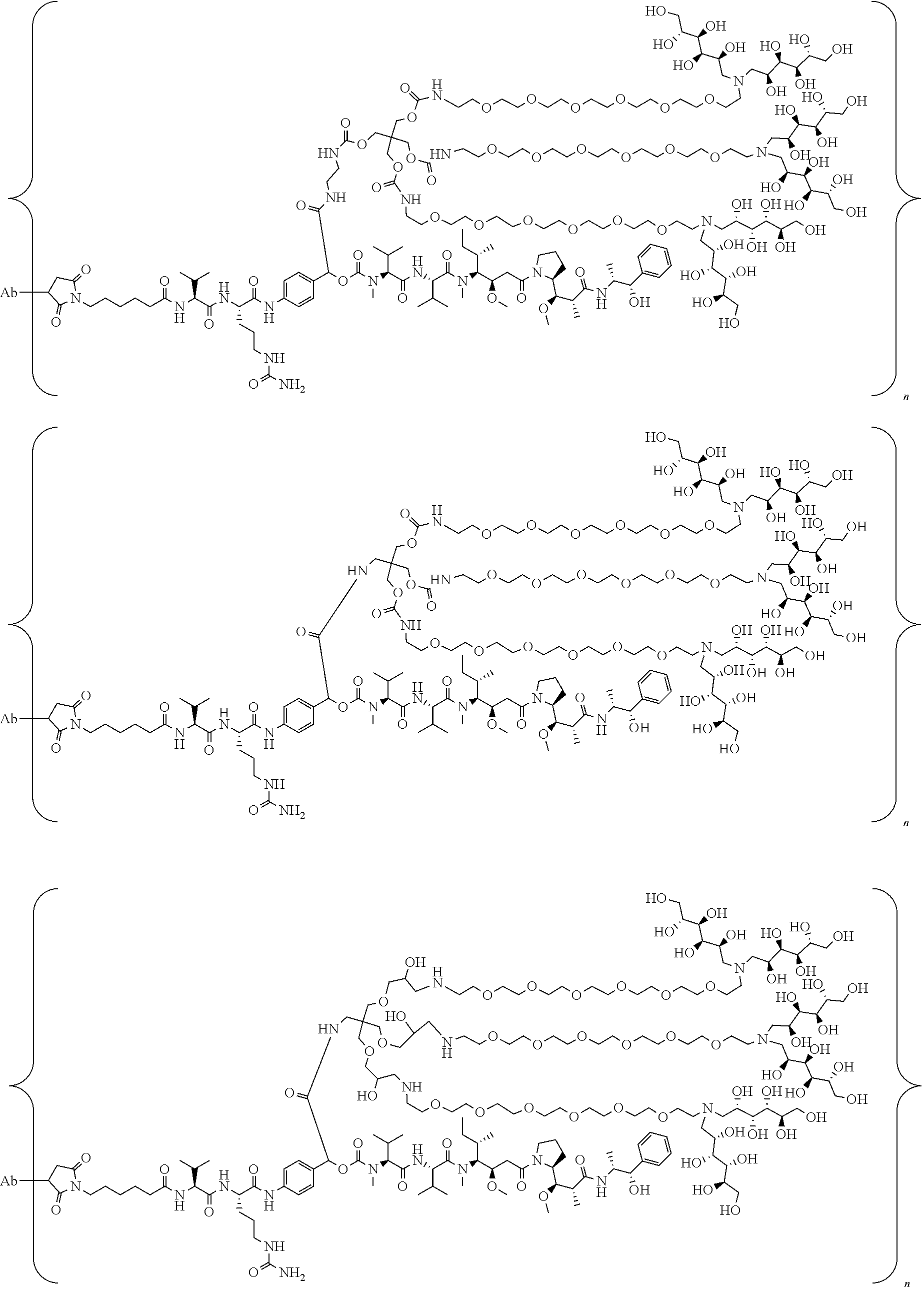

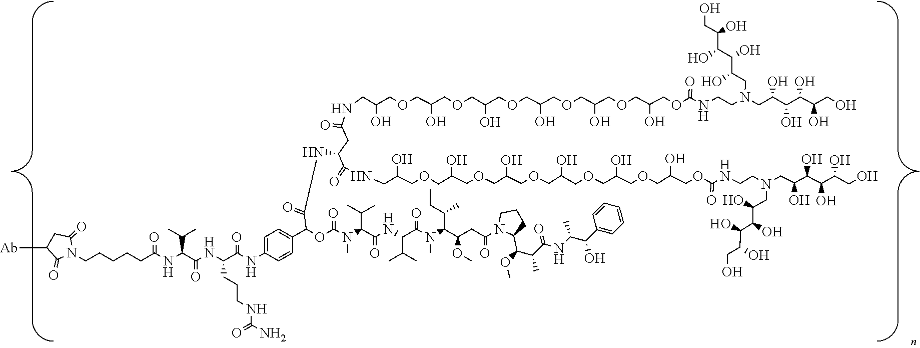

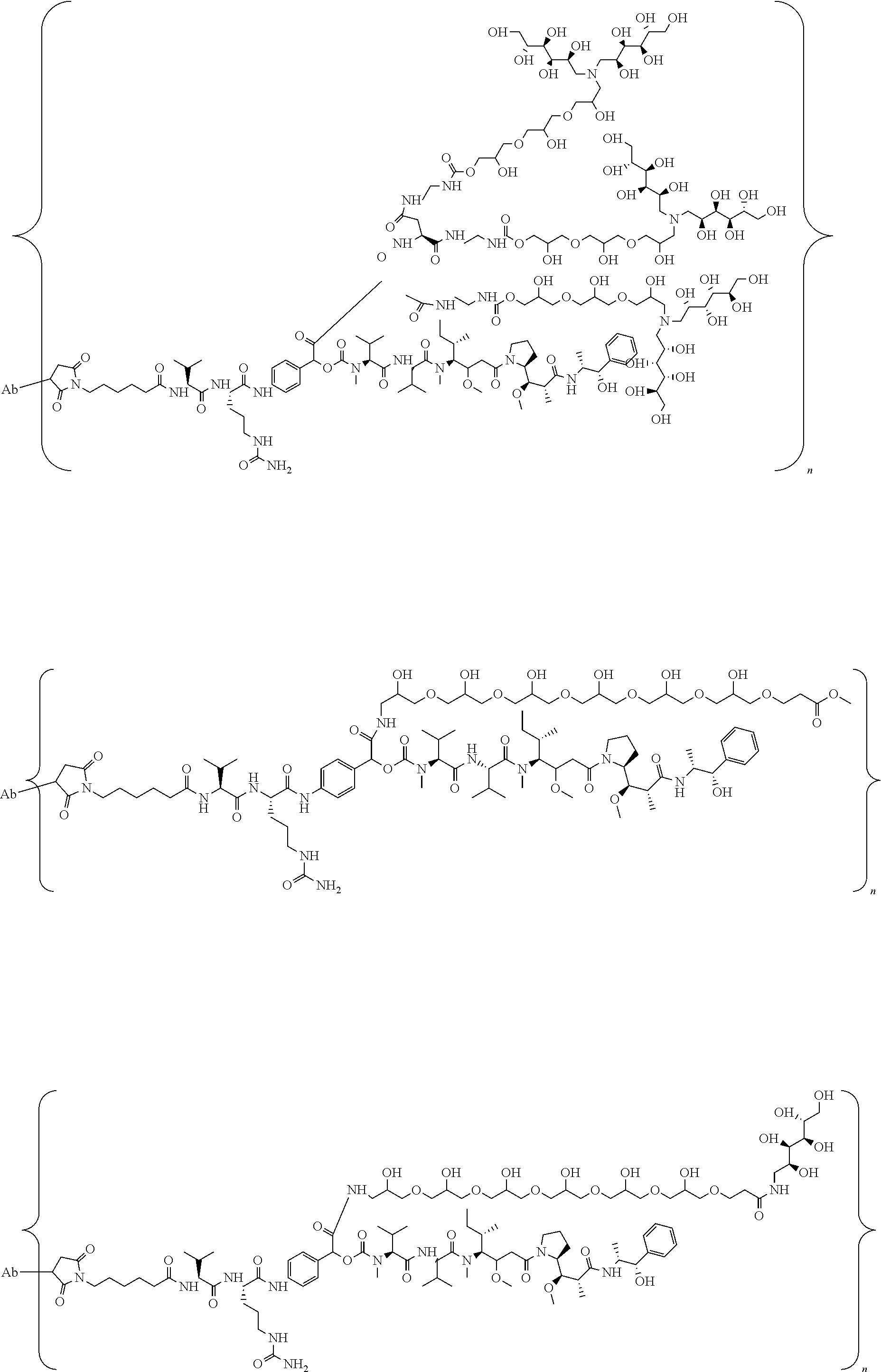

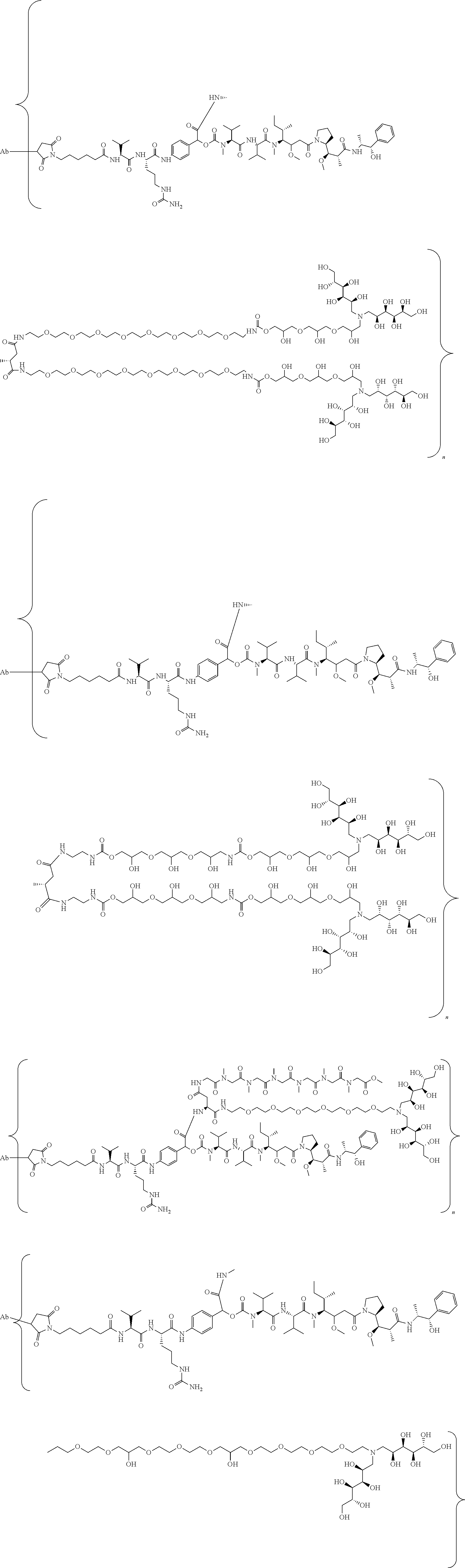

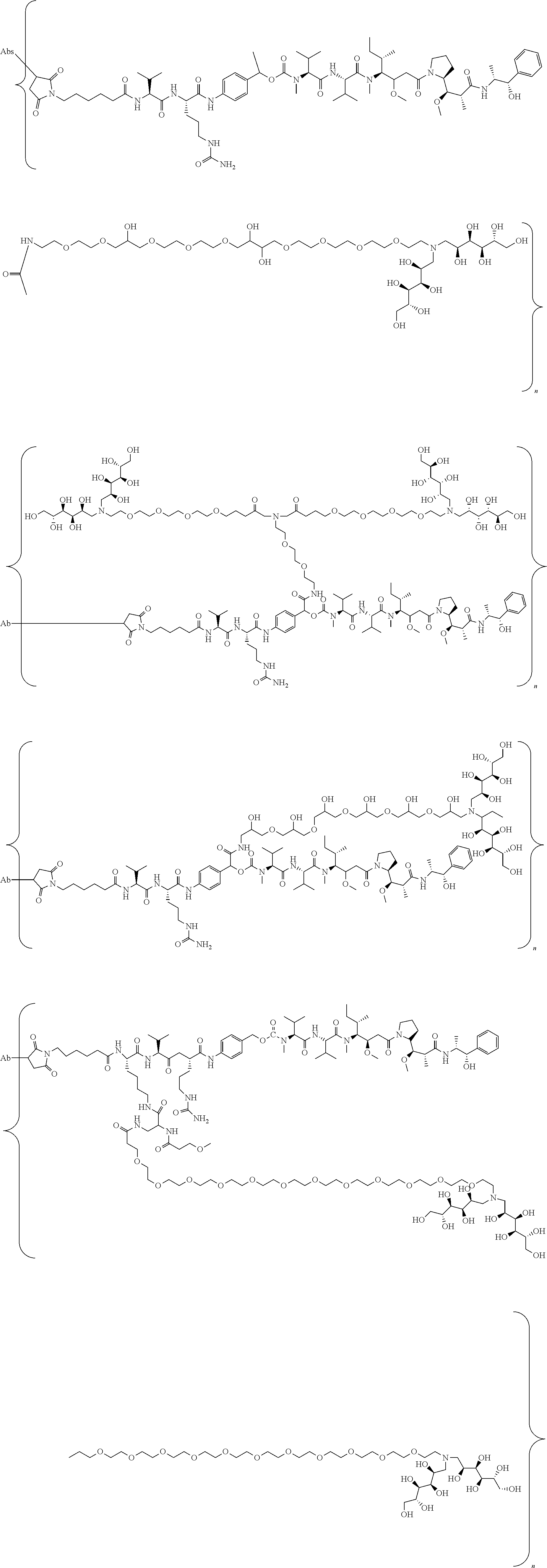

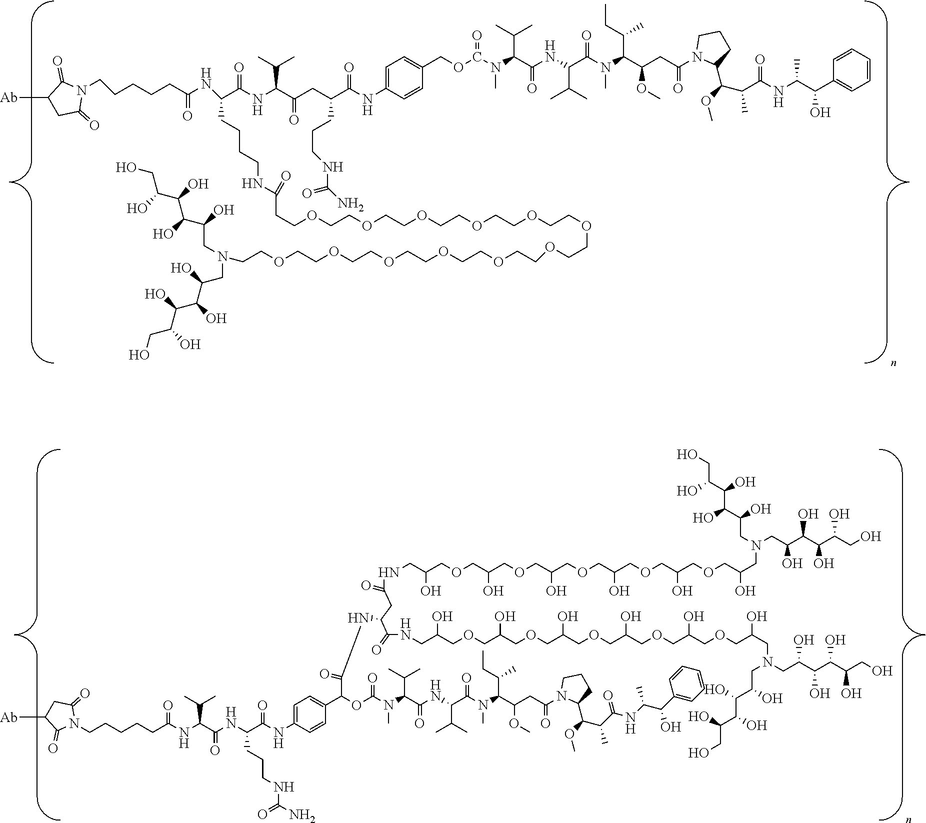

wherein Ab is a PTK7 binding agent and n is p^{load}.

In some embodiments, the conjugate is a stereoisomer of one of the above structures.

### Embodiment 67—Exemplary Conjugates

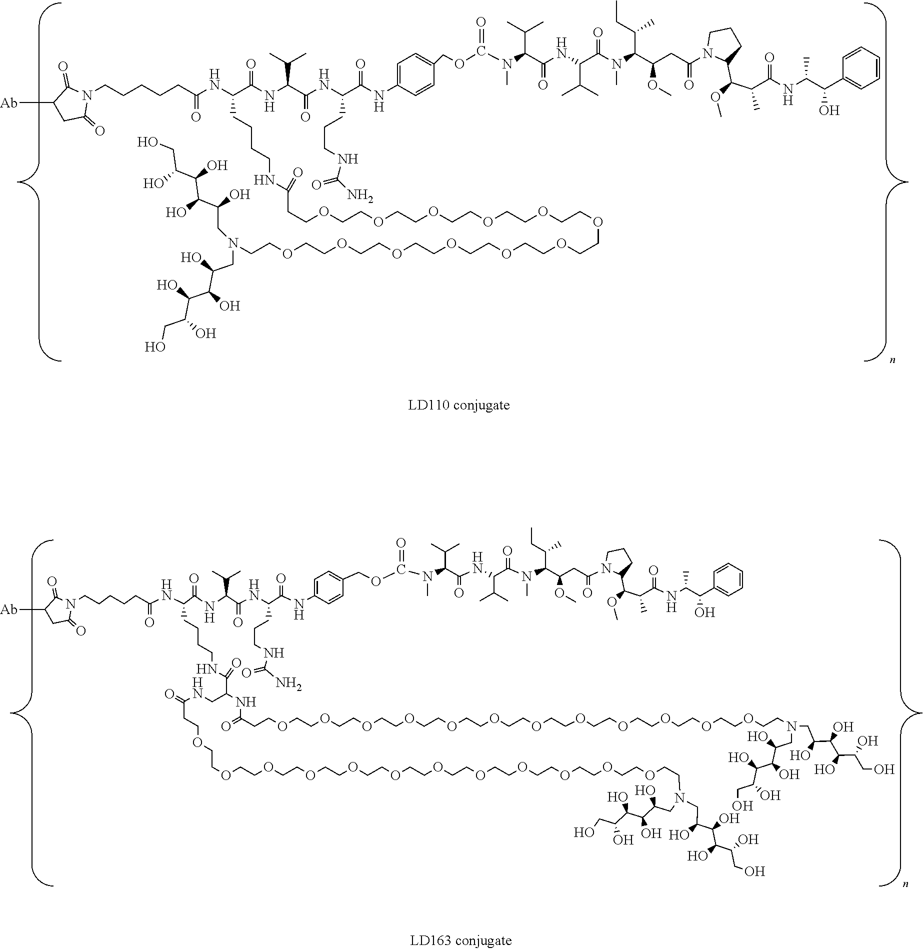

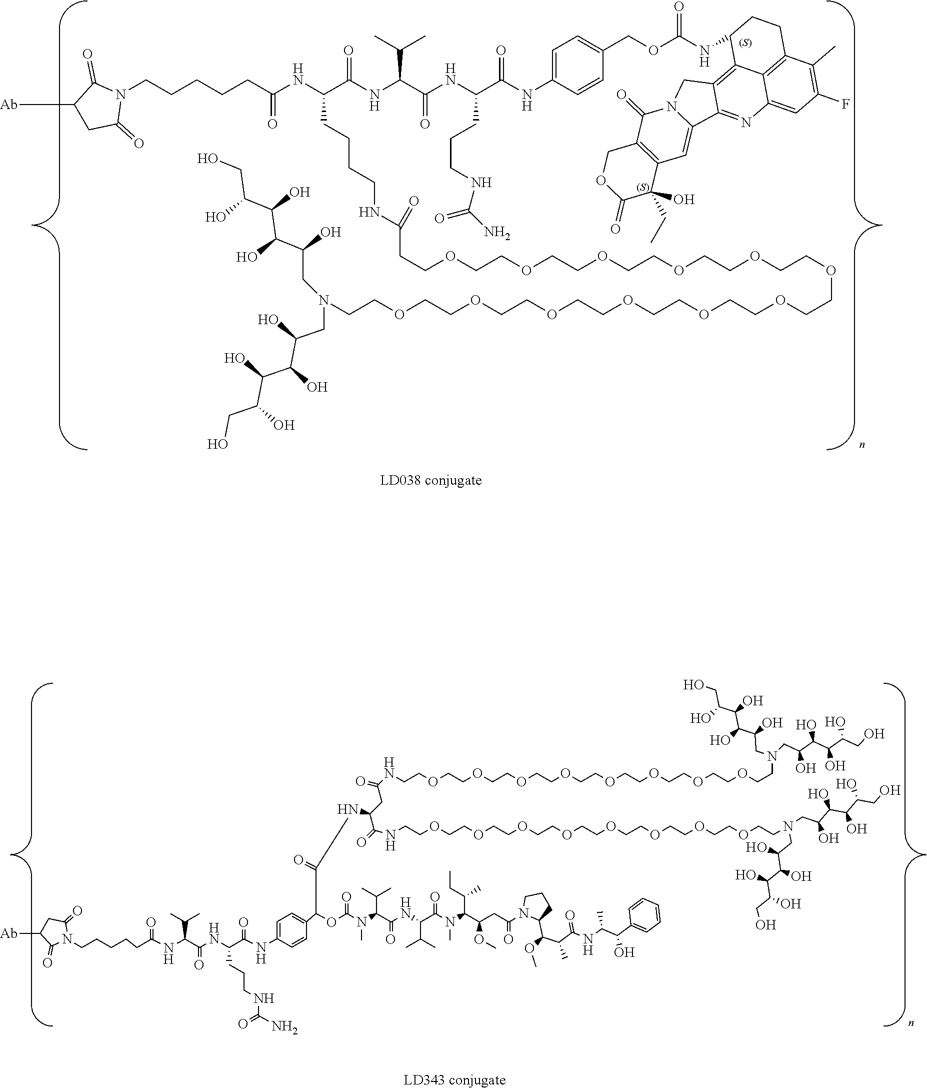

In some embodiments, the conjugate is a stereoisomer of one of the above structures.

### Embodiment 68—Exemplary Conjugate

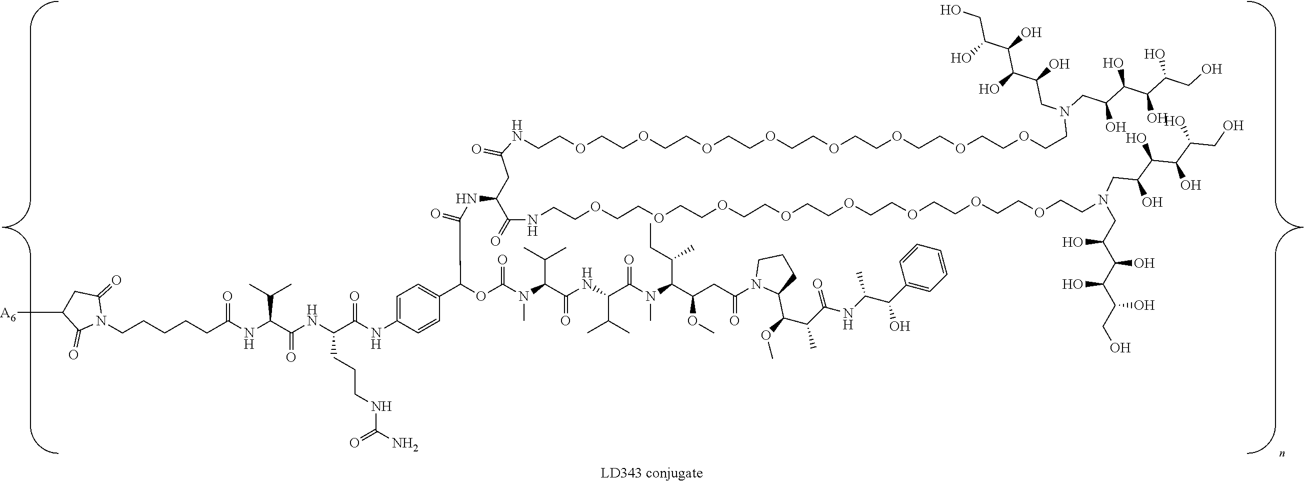

wherein Ab is a binding agent comprising a heavy chain having a heavy chain variable (VH) region and a heavy chain constant region and a light chain having a light chain variable (VL) region and a light chain constant region, the VH region having the amino acid sequence set forth in SEQ ID NO: 15, the VL region having the amino acid sequence set forth in SEQ ID NO: 16, the heavy chain constant region having the amino acid sequence set forth in SEQ ID NO: 56, and the light chain constant region having the amino acid sequence set forth in SEQ ID NO: 57, and n is p^{load} wherein p^{load} is 8.

In some embodiments, the conjugate is a stereoisomer of one of the above structures.

### Drug Loading

Conjugates can contain one or more drug unit per PTK7 binding agent. The number of drug units per PTK7 binding agent is referred to as drug loading. The drug loading of a Conjugate is represented by p^{load} the average number of drug units (drug molecules (e.g., cytotoxic agents)) per PTK7 binding agent (e.g., an antibody or antigen binding portion or non-antibody scaffold or non-antibody protein) in a conjugate. For example, if p^{load} is about 4, the average drug loading taking into account all of the PTK7 binding agent (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of drug units per PTK7 binding agent (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Attachment of Drug-Linkers to Antibodies, Antigen Binding Portions and Other Binding Agents (Including Non-Antibody Scaffolds)

Techniques for attaching drug unit(s) to PTK7 binding agent (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a linker is first attached to a drug unit (e.g., a cytotoxic agent(s), immune modulatory agent or other agent) and then the drug-linker(s) is attached to the PTK7 binding agent (e.g., an antibody or antigen binding portion thereof or non-antibody protein scaffold). In some embodiments, a linker(s) is first attached to a PTK7 binding agent (e.g., an antibody or antigen binding portion thereof or non-antibody protein scaffold), and then a drug unit is attached to a linker. In the following discussion, the term drug-linker is used to exemplify attachment of linkers or drug-linkers to the PTK7 binding agent; the skilled artisan will appreciate that the selected attachment method can be determined according to linker and the drug unit. In some embodiments, a drug unit is attached to a PTK7 binding agent via a linker in a manner that reduces the activity of the drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a PTK7 binding agent (e.g., an antibody or antigen binding portion thereof or non-antibody protein scaffold) with a bivalent linker to form a targeting group-linker intermediate via a covalent bond, followed by reaction with a drug unit; and (2) reaction of a nucleophilic group of a drug unit with a bivalent linker, to form drug-linker, via a covalent bond, followed by reaction with a nucleophilic group of a PTK7 binding agent. Exemplary methods for preparing conjugates via the latter route are described in U.S. Pat. No. 7,498,298, which is expressly incorporated herein by reference.

Nucleophilic groups on the PTK7 binding agent such as antibodies, antigen binding portions and other binding agents (including non-antibody scaffolds) include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. The PTK7 binding agent, such as antibodies (and antigen binding portions and other binding agents (including non-antibody scaffolds)) has reducible interchain disulfides, i.e., cysteine bridges. Antibodies (and antigen binding portions and other binding agents (including non-antibody scaffolds)) may be made reactive for conjugation with linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into the PTK7 binding agent such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into the PTK7 binding agent (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on the PTK7 binding agent, such as an aldehyde or ketone carbonyl group, with a nucleophilic group on a linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a linker. In another embodiment, the sugars of glycosylated antibodies may be oxidized, e.g. with periodate oxidizing reagents, to form aldehyde or ketone groups which may react with the amine group of a linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the linker (see, e.g., Hermanson, Bioconjugate Techniques). In another embodiment, the PTK7 binding agent such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a linker.

Exemplary nucleophilic groups on a drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a drug-linker is attached to an interchain cysteine residue(s) of an antibody (or antigen binding portion thereof or other binding agent (including non-antibody scaffolds)). See, e.g., WO2004/010957 and WO2005/081711. In such embodiments, the linker typically comprises a maleimide group for attachment to the cysteine residues of an interchain disulfide. In some embodiments, a linker or drug-linker is attached to a cysteine residue(s) of an antibody or antigen binding portion thereof as described in U.S. Pat. Nos. 7,585,491 or 8,080,250. The drug loading of the resulting conjugate typically ranges from 1 to 8 or 1 to 16.

In some embodiments, a linker or drug-linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a drug-linker(s) is attached to an engineered cysteine residue at an Fc residue other than an interchain disulfide. In some embodiments, a drug-linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or drug-linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or drug-linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014//065661, WO2015/057066 and WO2016/022027; the disclosure of which are incorporated by reference herein.

In some embodiments, a linker or drug-linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 61) to an N-terminal GGG motif to regenerate a native amide bond.

In some embodiments, a linker or drug-linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a linker or drug-linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a linker or drug-linker is attached to an antibody, antigen binding portion or other binding agent (including non-antibody scaffolds) using bis-maleimide, C-lock, or K-lock methodologies.

### Pharmaceutical Compositions

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate).

The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment Methods

In some embodiments, provided are methods of treating a subject, comprising administering to the subject a conjugate described herein or a pharmaceutical composition described herein. For example, in some embodiments the subject has cancer or an autoimmune disease and the conjugate binds to the target antigen associated with the cancer or autoimmune disease. Also provided is a conjugate described herein or a pharmaceutical composition described herein for use in treating a subject, comprising administering to the subject said conjugate or pharmaceutical composition.

In some embodiments, provided are methods of treating cancer comprising administering a conjugate. In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount”, “effective amount” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the PTK7+ cancer is a carcinoma. In some embodiments, the cancer is a squamous cell carcinoma.

In some embodiments, the cancer is a PTK7+ cancer. In some embodiments, the PTK7+ cancer is a solid tumor or a hematologic malignancy.

In some embodiments, the PTK7+ cancer is selected from breast cancer, ovarian cancer (OVCA), cervical cancer, pharynx cancer, stomach cancer, myeloma, bladder cancer, uterine cancer, esophageal squamous cell carcinoma (ESCC), colon cancer, hepatocellular cancer, and colorectal cancer.

In some embodiments, the PTK7+ cancer is selected from esophageal squamous cell carcinoma (ESCC), lung cancer and head and neck squamous cell carcinoma (HNSCC).

In some embodiments, the cancer is selected from bladder cancer, esophageal cancer and uterine cancer.

In some embodiments, the PTK7+ cancer is selected from triple negative breast cancer (TNBC), non-small cell lung cancer (NSCLC), gastroesophageal cancer, and urothelial cancer.

In some embodiments, the PTK7+ cancer is a hematologic malignancy.

In some embodiments, PTK7+ cancer is a solid tumor. In some embodiments, the PTK7+ cancer is triple-negative breast cancer (TNBC). In some embodiments, the PTK7+ cancer is non-small-cell lung cancer (NSCLC). In some embodiments, the PTK7+ cancer is ovarian teratocarcinoma. In some embodiments, the PTK7+ cancer is pharynx cancer. In some embodiments, the PTK7+ cancer is gastric adenocarcinoma. In some embodiments, the PTK7+ cancer is endometrial adenocarcinoma. In some embodiments, the PTK7+ cancer is bladder transitional cell carcinoma. In some embodiments, the PTK7+ cancer is bladder transitional cell papilloma. In some embodiments, the PTK7+ cancer is ESCC.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%. The methods set out herein may slow tumor growth.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount”, “effective amount” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods described herein encompass treatment of disorders of B lymphocytes (e.g., systemic lupus erythematosus, Goodpasture's syndrome, rheumatoid arthritis, and type I diabetes), Th1-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune diseases. In addition, the methods described herein can be used to treat domesticated animals and/or pets. A subject can be male or female. In certain embodiments, the subject is a human.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, reduction in autoimmune cells in the subject, alleviation of one or more symptom(s), diminishment of extent of the deficit, stabilized (i.e., not worsening) state of an autoimmune disease, delay or slowing of progression of an autoimmune disease, and an increased lifespan as compared to that expected in the absence of treatment. As used herein, the term “administering,” refers to providing a conjugate as described herein to a subject by a method or route which results in binding of the conjugate to target autoimmune cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate thereof can be administered in a unit dose. The term “unit dose” when used in reference to a pharmaceutical composition refers to physically discrete units suitable as unitary dosage for the subject, each unit containing a predetermined quantity of active material (e.g., a conjugate), calculated to produce the desired therapeutic effect in association with the required physiologically acceptable diluent, i.e., carrier, or vehicle.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Abbreviations

Boc₂O: di-tert-butyl dicarbonate
Bu₄NBr: Tetrabutylammonium bromide
DCM: dichloromethane
DEA: Diethanolamine
DEAD: diethyl azodicarboxylate
DIPEA: N,N-diisopropylethylamine
DMAP: 4-(Dimethylamino)pyridine
DMF: N,N-dimethylformamide
DMTMM: 4-(4,6-dimethoxy-1,3,5-triazin-2-yl)-4-methyl-morpholinium chloride
DMSO: dimethylsulfoxide
EEDQ: N-Ethoxycarbonyl-2-ethoxy-1,2-dihydroquinoline
ESI: electrospray ionization
HATU: 1-[bis(dimethylamino)methylene]-1H-1,2,3-triazolo[4,5-b]pyridinium 3-oxidhexa fluorophosophate)
HOBt: hydroxylbenzotriazole
LCMS: liquid chromatography-mass spectrometry
MeCN: acetonitrile
MeOH: methanol
m-CPBA: meta-chloroperoxybenzoic acid
MTBE: Methyl tert-butyl ether
NMR: nuclear magnetic resonance spectroscopy
Ph₃CCl: Triphenylmethyl chloride
PNPC: bis(4-nitrophenyl) carbonate
PPh₃: triphenylphosphine
TFA: trifluoroacetic acid
THF: tetrahydrofuran
TLC: thin-layer chromatography
TsOH: p-Toluenesulfonic acid

### General Methods

¹H NMR and other NMR spectra were recorded on BrukerAVIII 400 or BrukerAVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 um; Column Temperature: 40° C. Detectors: ADC ELSD, DAD (214 nm and 254 nm), ES-API.

Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.

Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:

Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.

Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:

Method A: Waters SunFire 10 μm C18 column (100 Å, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.

Method B: Waters SunFire 10 μm C18 column (100 Å, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.

Method C: Waters Xbridge 10 μm C18 column (100 Å, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of Drug-Linker 1

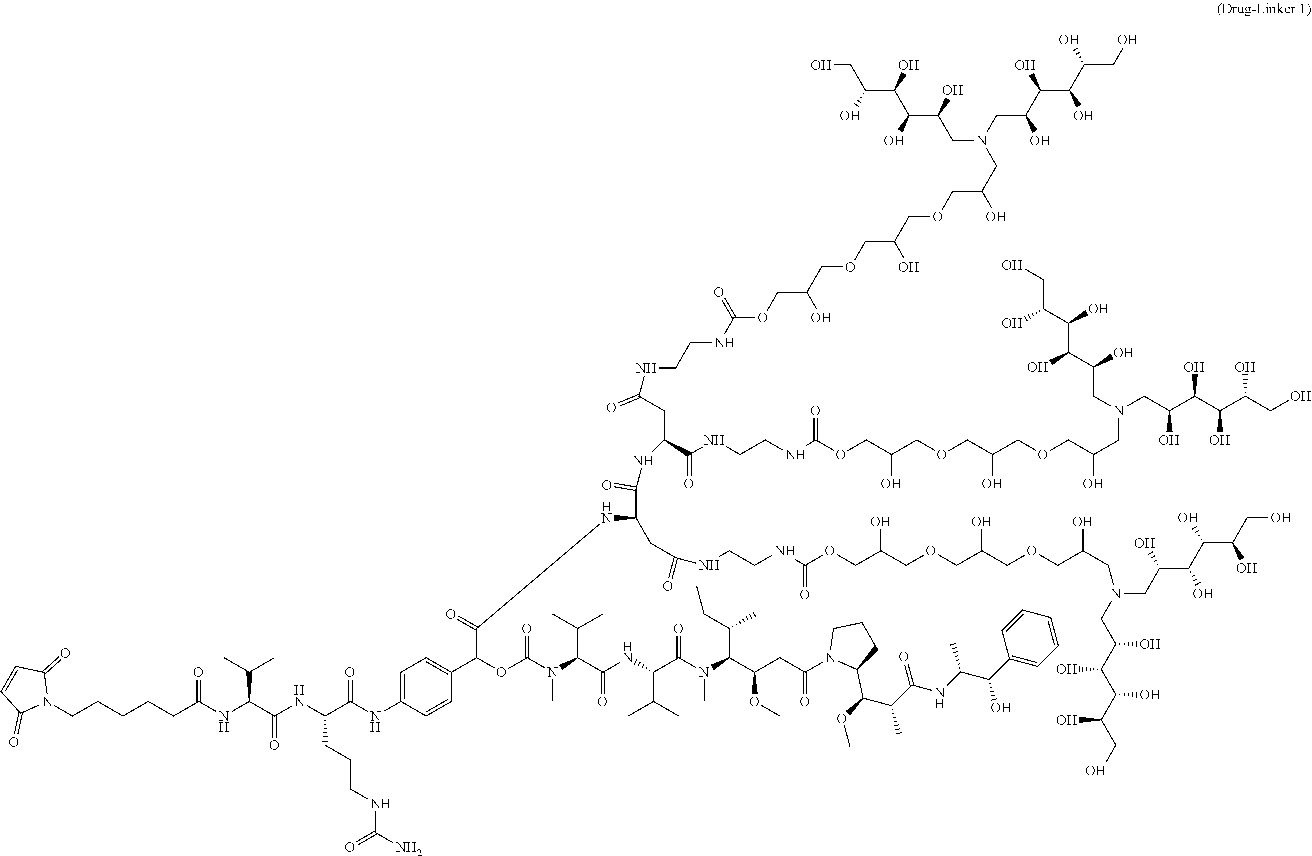

### Step 1

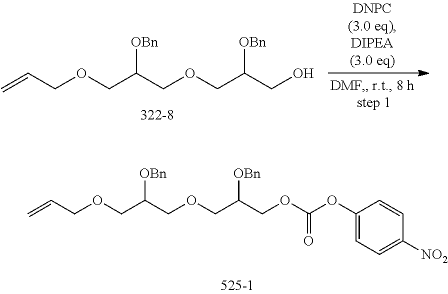 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 322-8 was consumed as detected by LCMS. The reaction solution was directly used in the next step without a work-up procedure.

### Step 2

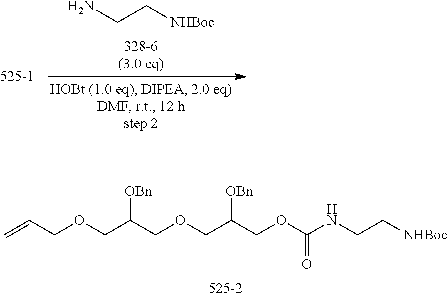 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed as detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

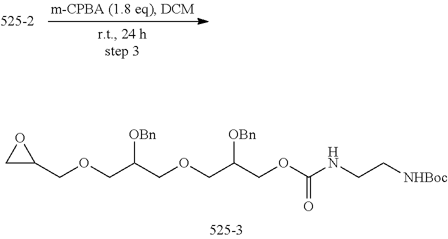 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, VN), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil. Purity=90%-95%.

### Step 4

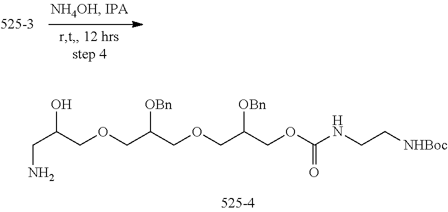 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

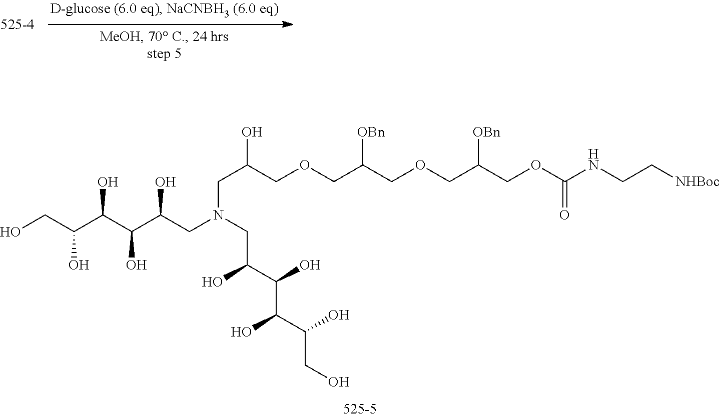 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

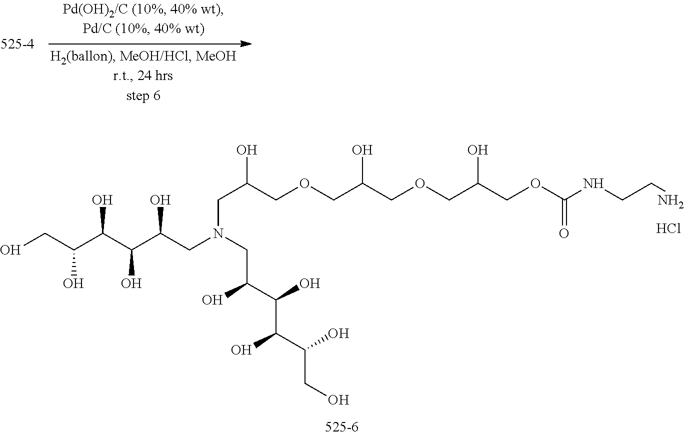 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (balloon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

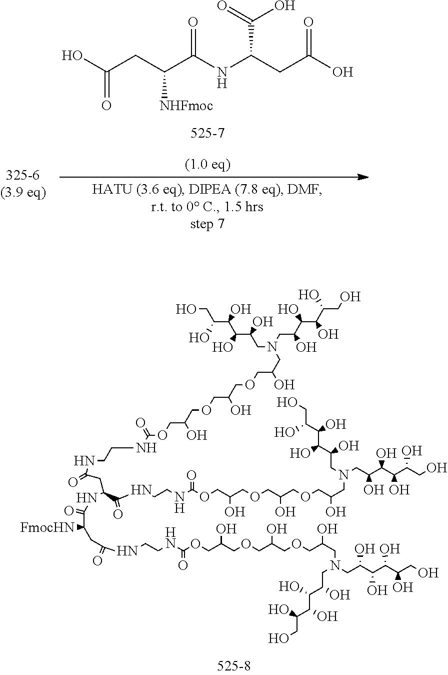

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 10H), 4.08-3.87 (m, 12H), 3.84-3.69 (m, 18H), 3.67-3.35 (m, 53H), 3.30-3.07 (m, 12H), 2.77-2.32 (m, 4H).

### Step 8

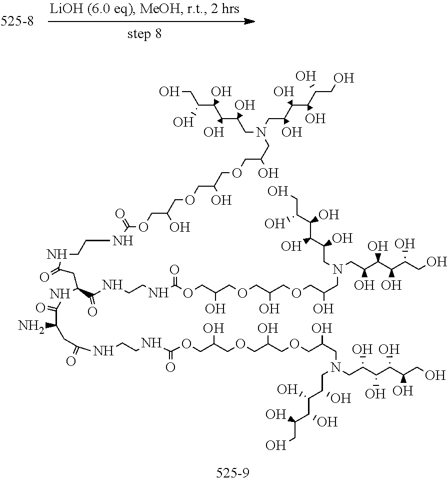 [SMILES: N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1 N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

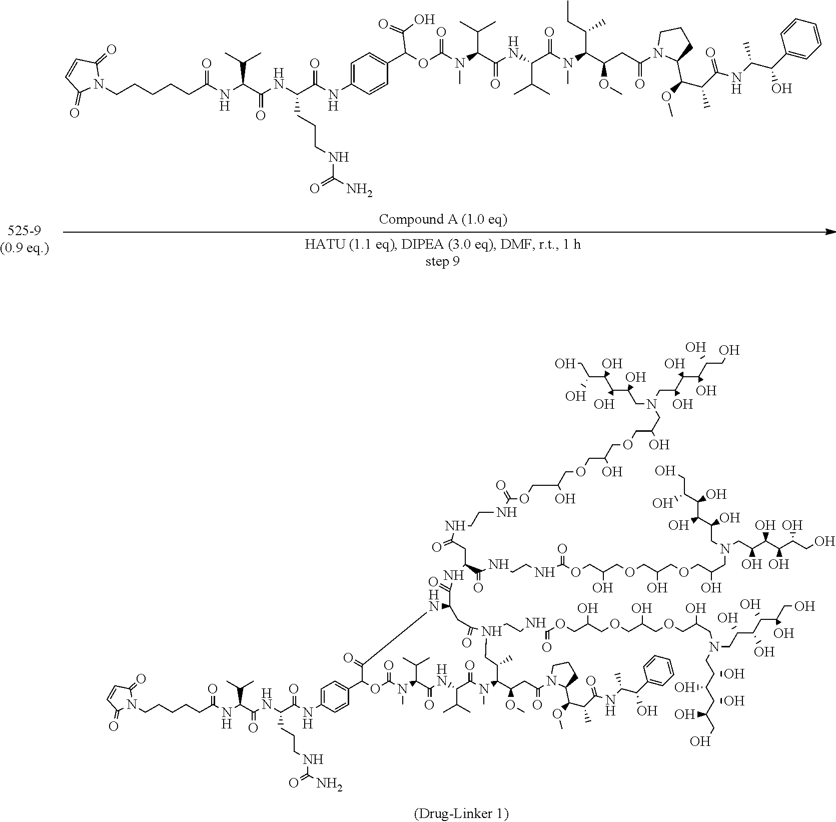

A solution of 525-9 (70.0 mg, 0.0325 mmol), Compound A (49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give drug-linker 1 (23.5 mg, 0.00672 mmol, 20.7%) as a white solid. LCMS, m/z=1749.66 (M/2+H)⁺, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 2: Preparation of Drug-Linker 2

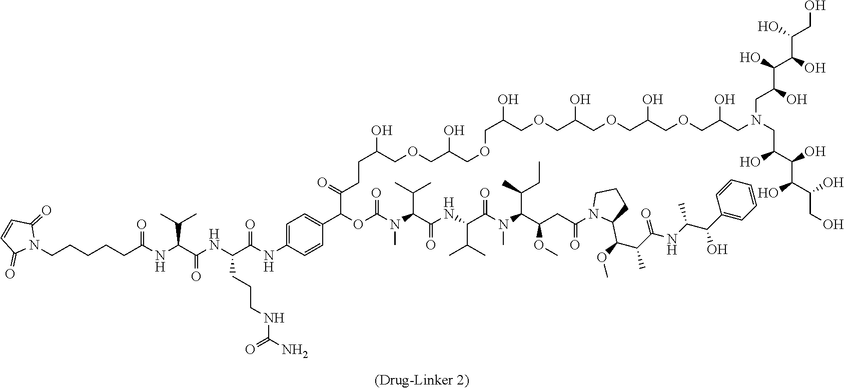 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)CCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

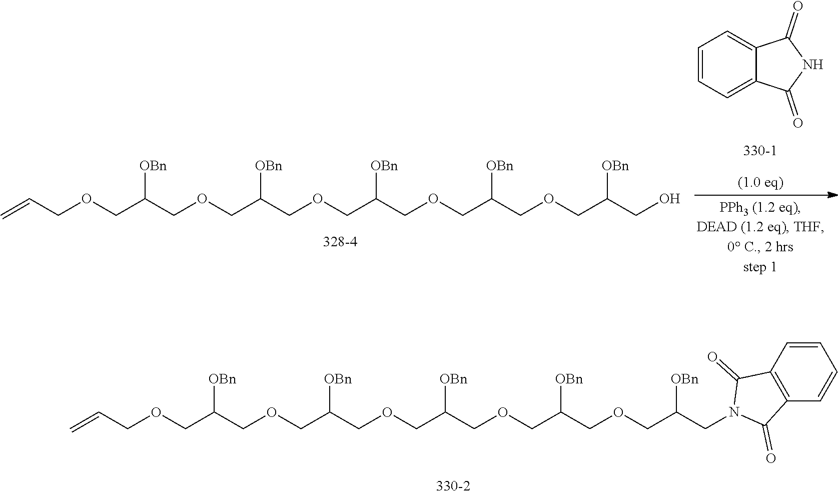 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THE (8 mL) was added a solution of 328-4 (1.3 g, 1.48 mmol) in THE (4 mL) and the mixture was stirred in an ice bath. A solution of DEAD (309.3 mg, 1.776 mmol) in THE (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. ¹H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

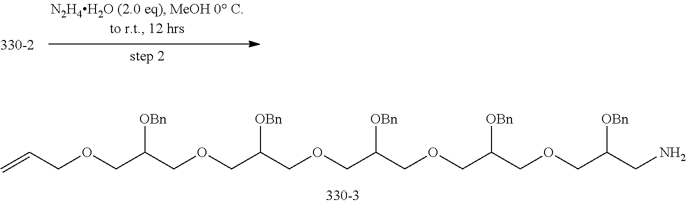 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

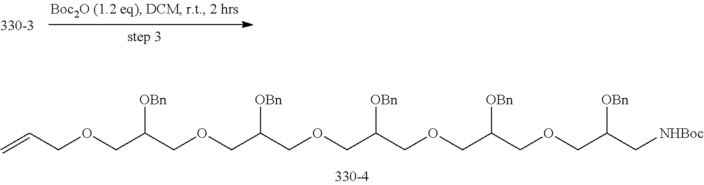 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

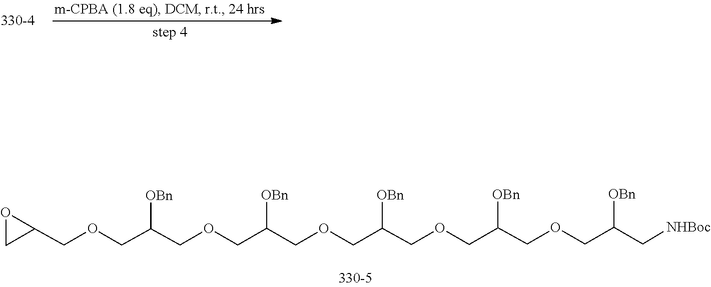 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequencely washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

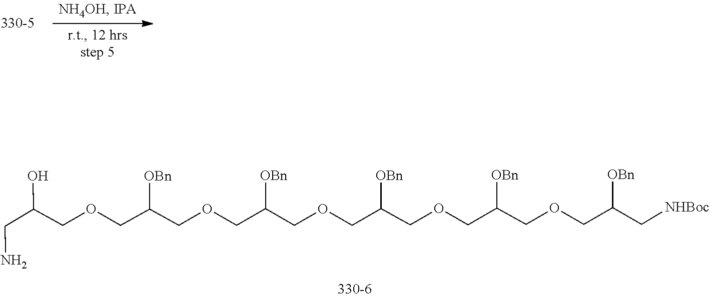 [SMILES: CC(C)(C)OC(=O)NCC(COCC(COCC(COCC(COCC(COCC(O)CN)OCc1ccccc1)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

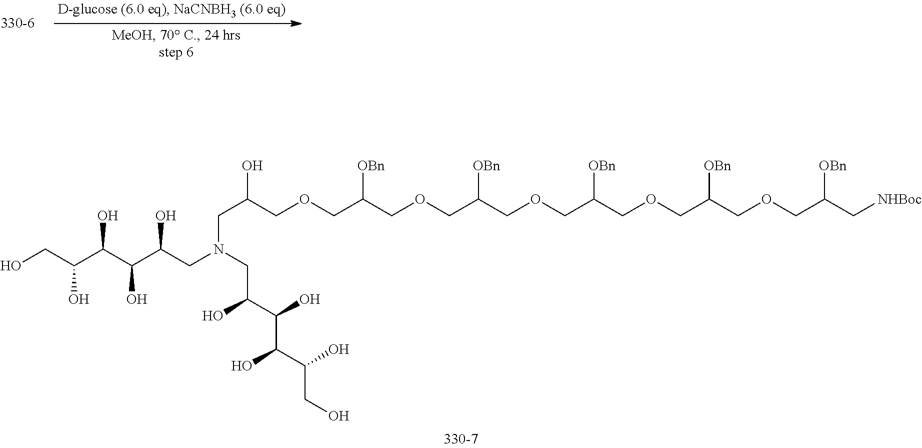 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

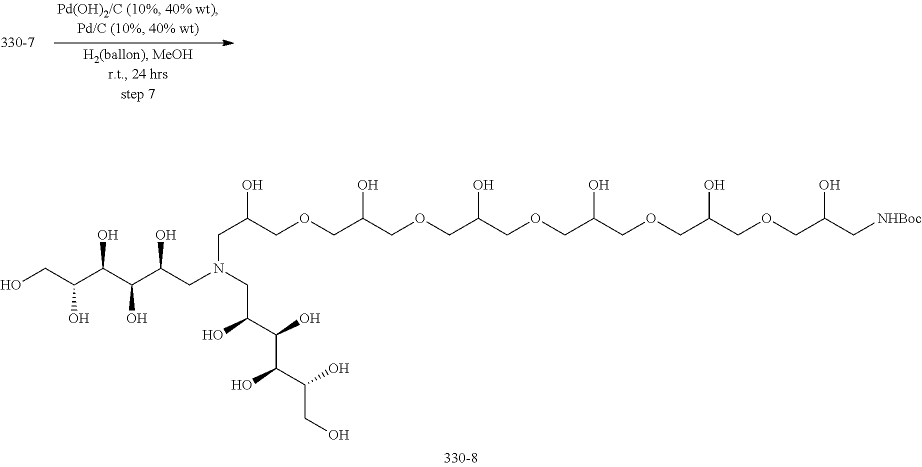 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (balloon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

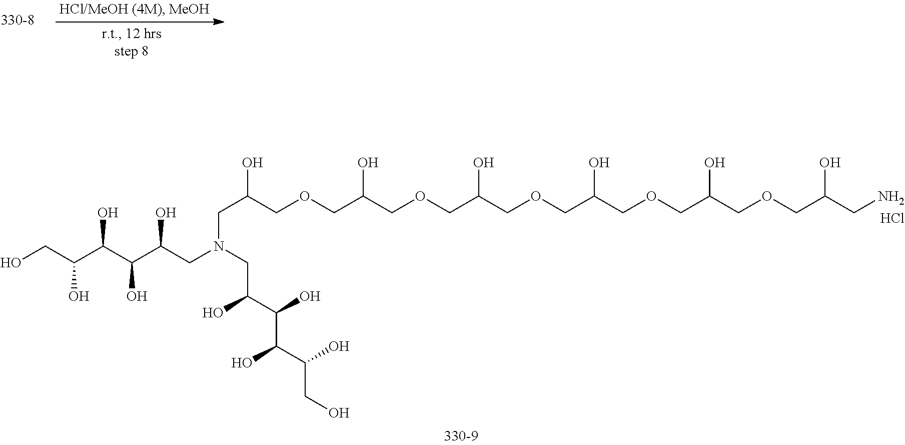 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

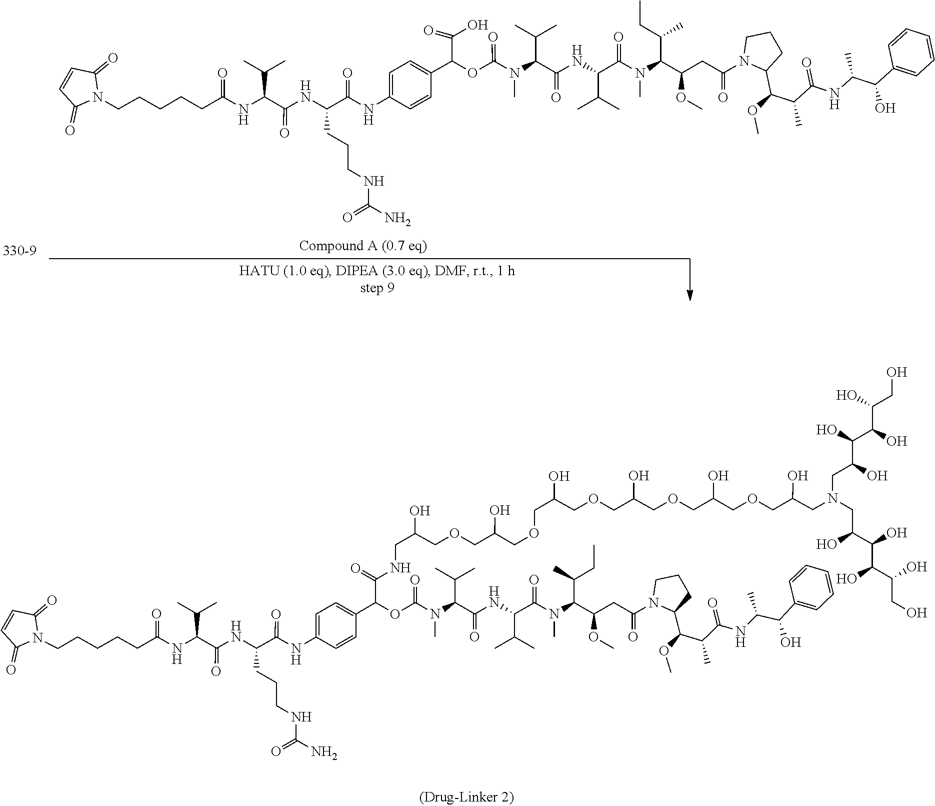

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give drug-linker 2 (28 mg, 0.0131 mmol. 33.7%) as a white solid. LCMS, m/z=1066.11 (M/2+H)⁺. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 3: Preparation of Drug-Linker 3

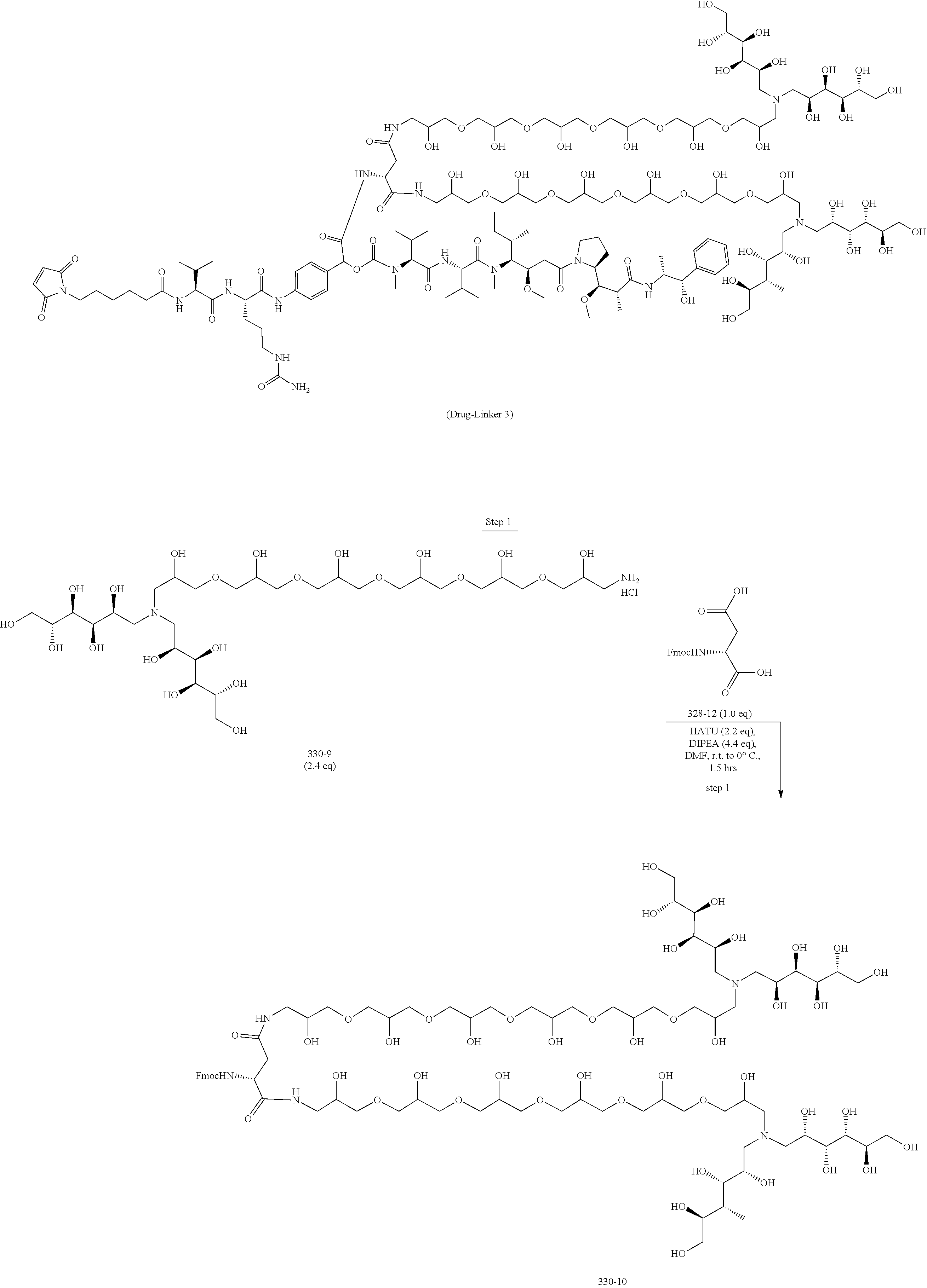

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 2

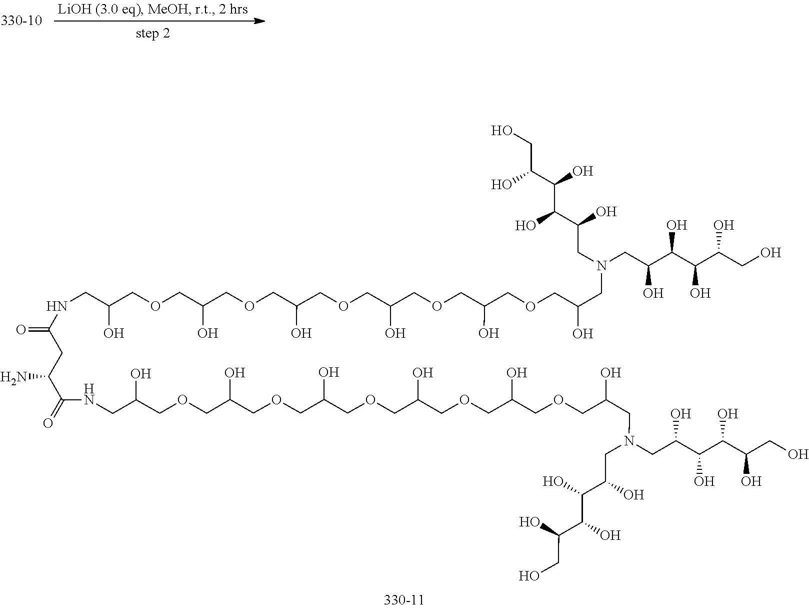 [SMILES: N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1 N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 3

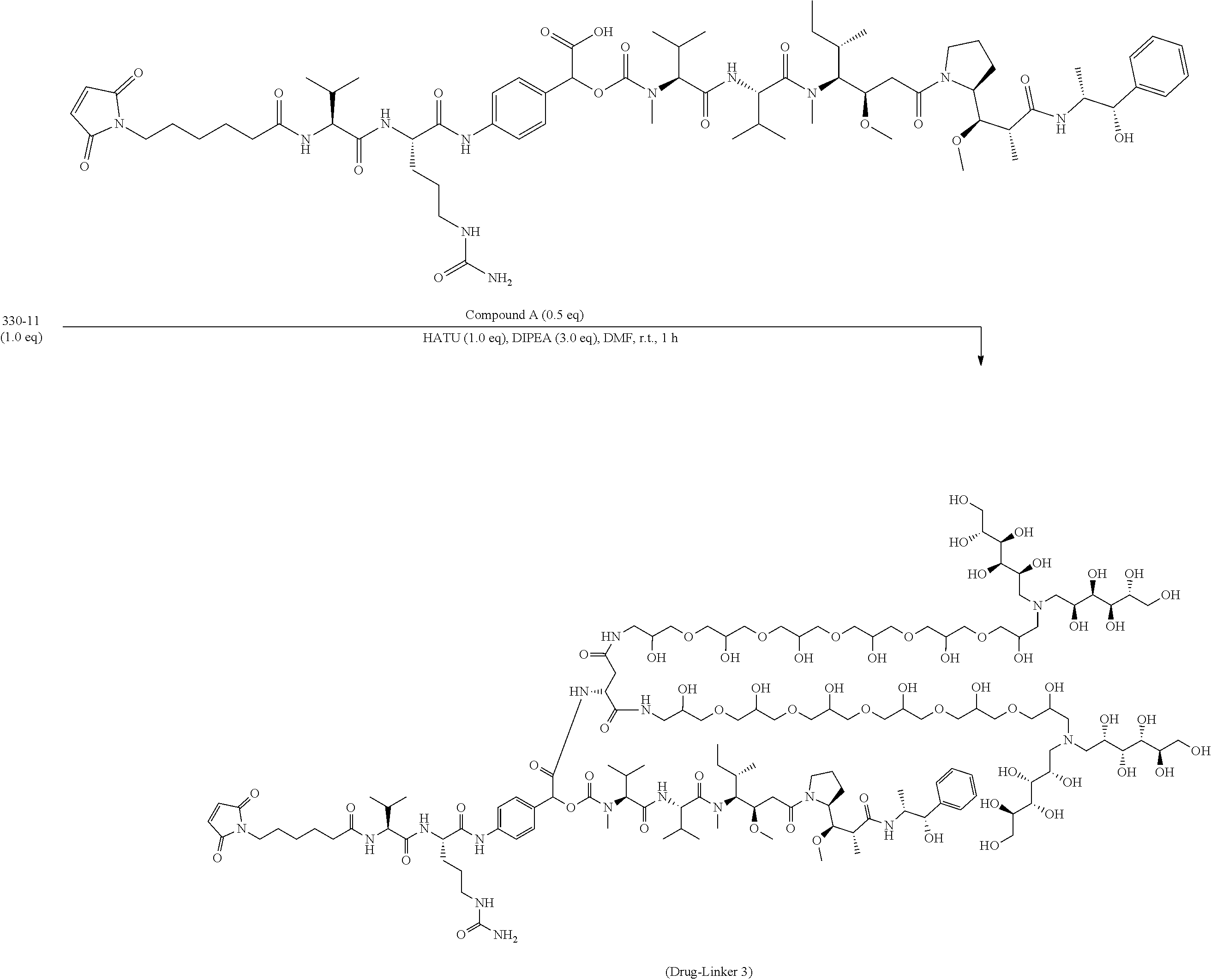

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give drug-linker 3 (5.0 mg, 0.00166 mmol, 45.1%) as a white solid. LCMS, m/z=1509.00 (M/2+H)⁺, m/z=1006.6 (M/3+H)⁺.

### Example 4: Preparation of Drug-Linker 4

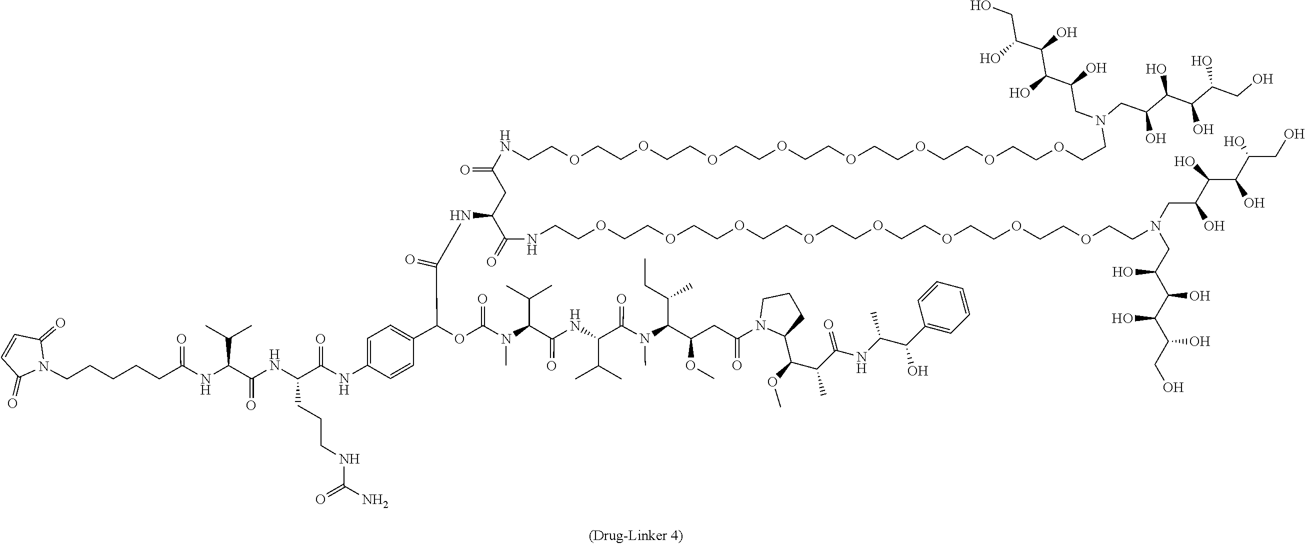

### Step 1

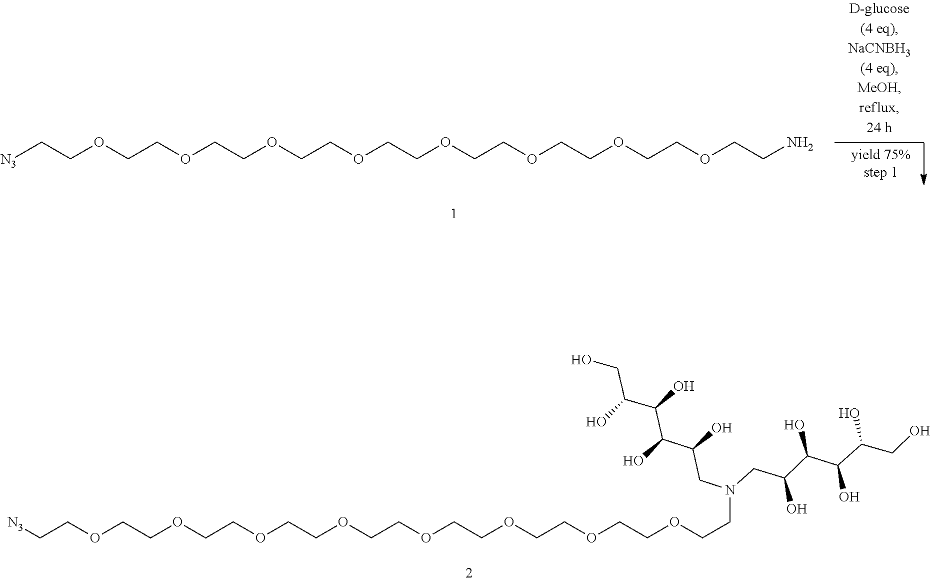 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 1 (2.5 g, 5.701 mmol) in MeOH (20 mL) was added D-Glucose (4.11 g, 22.804 mmol) and NaBH₃CN (1.385 mL, 22.804 mmol). The mixture was stirred at reflux for 24 h to complete. Then the resulting solution concentrated to dryness and the residue was purified by reverse phase chromatography (C₈ column, eluting with 0-45% methanol in water with 0.01% TFA) to afford the product 2 as yellow oil. ESI m/z: 767.5 (M+H)⁺.

### Step 2

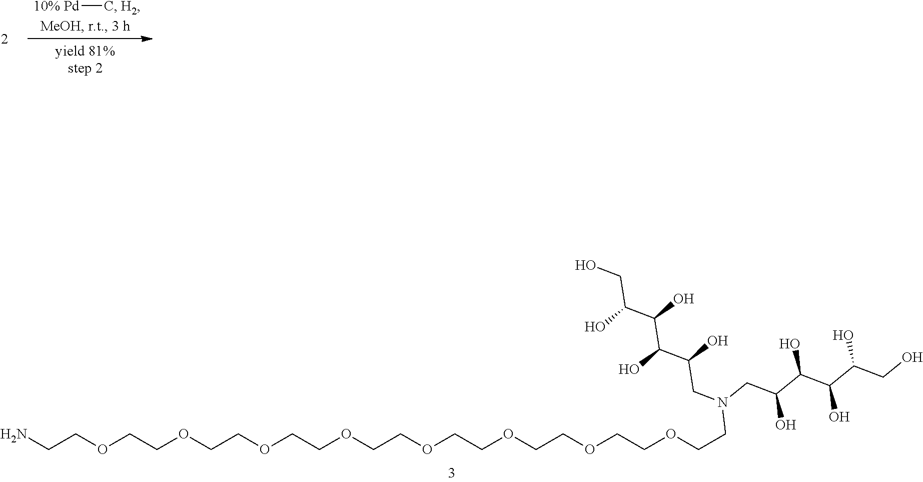

To the solution of 2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3 h to complete. The resulting mixture was filtered to remove catalyst solid and the filtrate was concentrated, then purified by reverse phase chromatography (C8 column, eluting with 0-25% acetonitrile in water with 0.01% TFA) to afford the product 3 (2.6 g, 3.510 mmol, 81.50%) as colorless oil. ESI m/z: 371.3 (M/2+H)+, 741.4 (M+H)⁺.

### Step 3

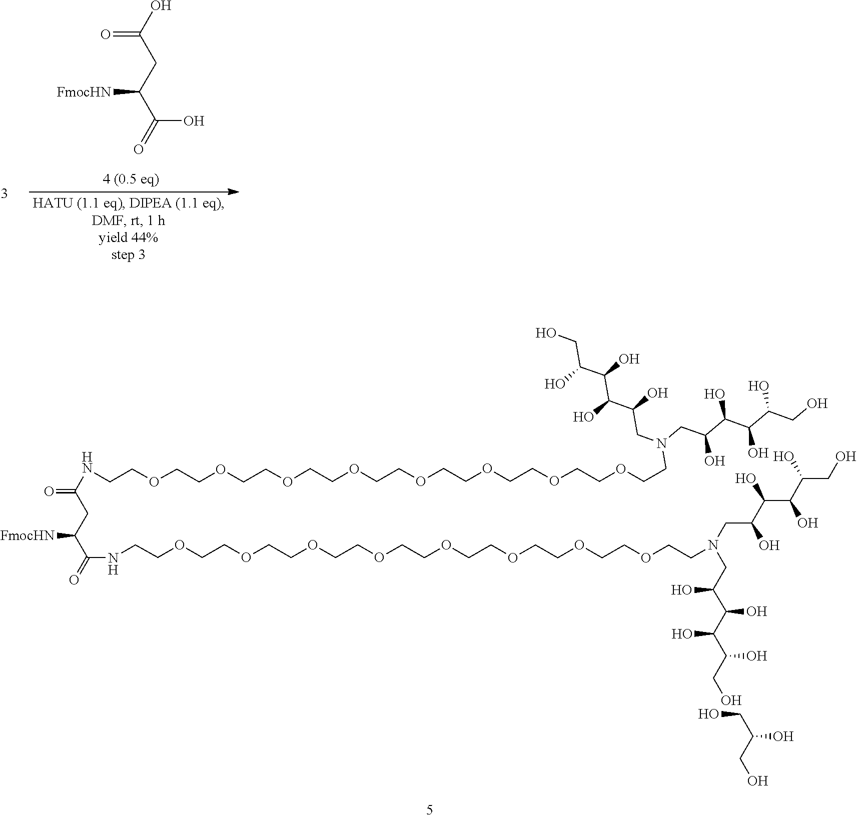

A solution of compound 5 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 3 (2.6 g, 3.510 mmol) in DMF (5 mL). After addition, the solution was stirred at room temperature for another 1 h to complete. The completed solution was then purified directly by reverse phase chromatography (C8 column, eluting with 0-40% acetonitrile in water with 0.01% TFA) to afford the product 5 (1.4 g, 0.777 mmol, 44.30%) as colorless oil. ESI m/z: 601.0 (M/3+H)⁺, 901.0 (M/2+H)⁺.

### Step 4

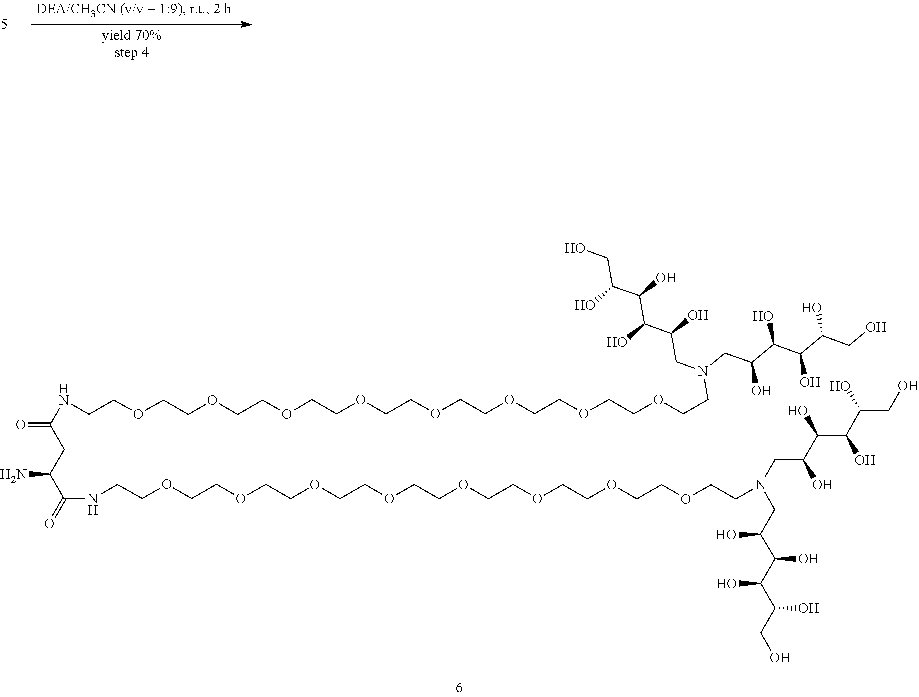

To the solution of 5 (1.4 g, 0.777 mmol) in MeCN (6 mL) was diethyl amine (0.7 mL, 8.930 mmol). The mixture was stirred at room temperature for 2 h to achieve complete deprotection. Then the resulting solution was concentrated under reduced pressure to remove most of diethyl amine, and the residue was purified by reverse phase chromatography (C₈ column, eluting with 0-20% acetonitrile in water with 0.01% TFA) to get desired fractions, which was freeze-dried to afford 6 as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

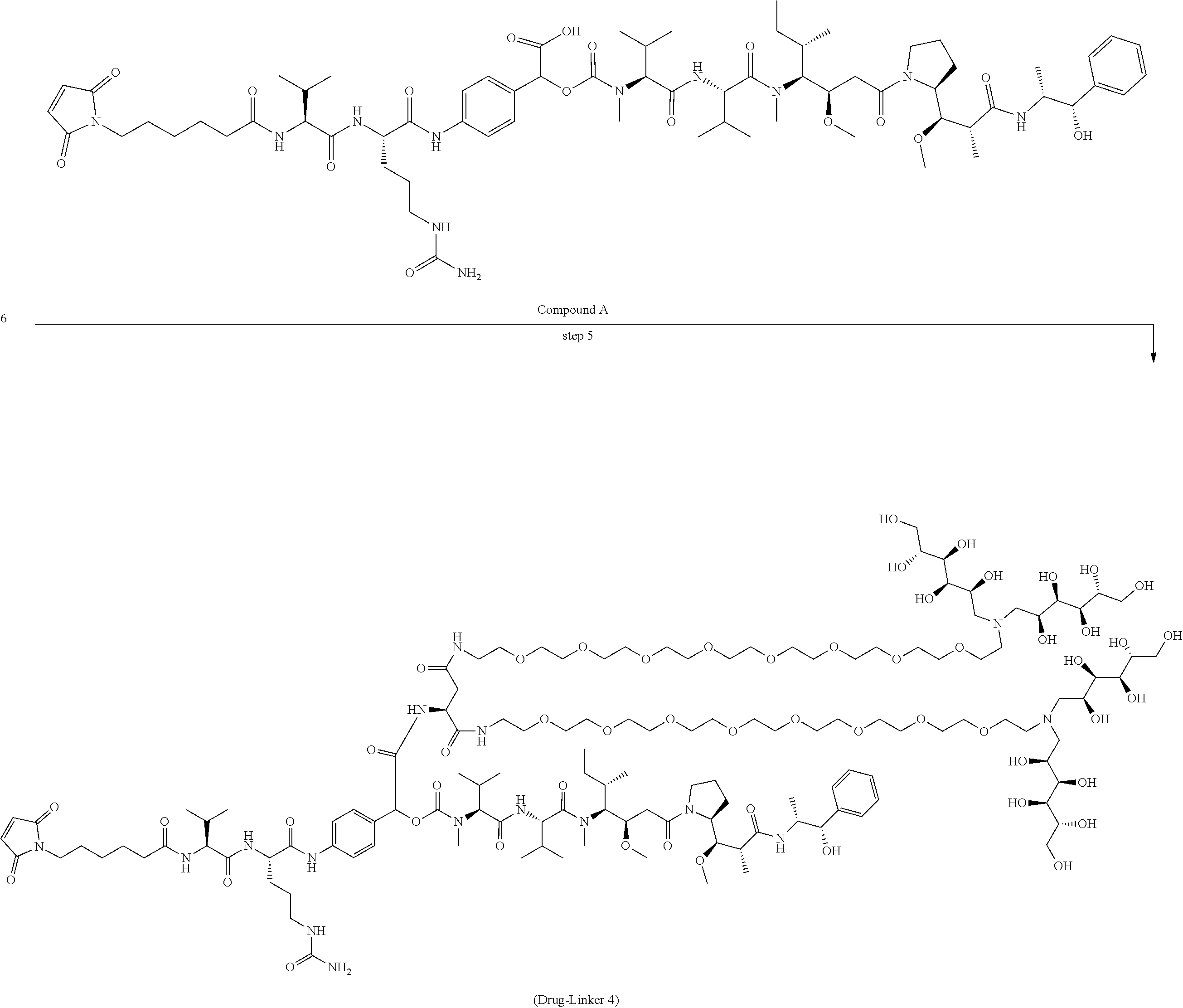

A solution of 6 (100 mg, 0.063 mmol), Compound A (86 mg, 0.063 mmol) and HATU (24 mg, 0.063 mmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give drug-linker 4 (40 mg, 0.014 mmol, 21.73%) as a white solid. LCMS, m/z=1461.23 (M/2+H)⁺; ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 10H), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 10H), 1.24-1.08 (m, 9H), 1.02-1.01 (m, 2H), 0.92-0.75 (m, 28H) ppm.

### Example 5: Preparation of Drug-Linker 5

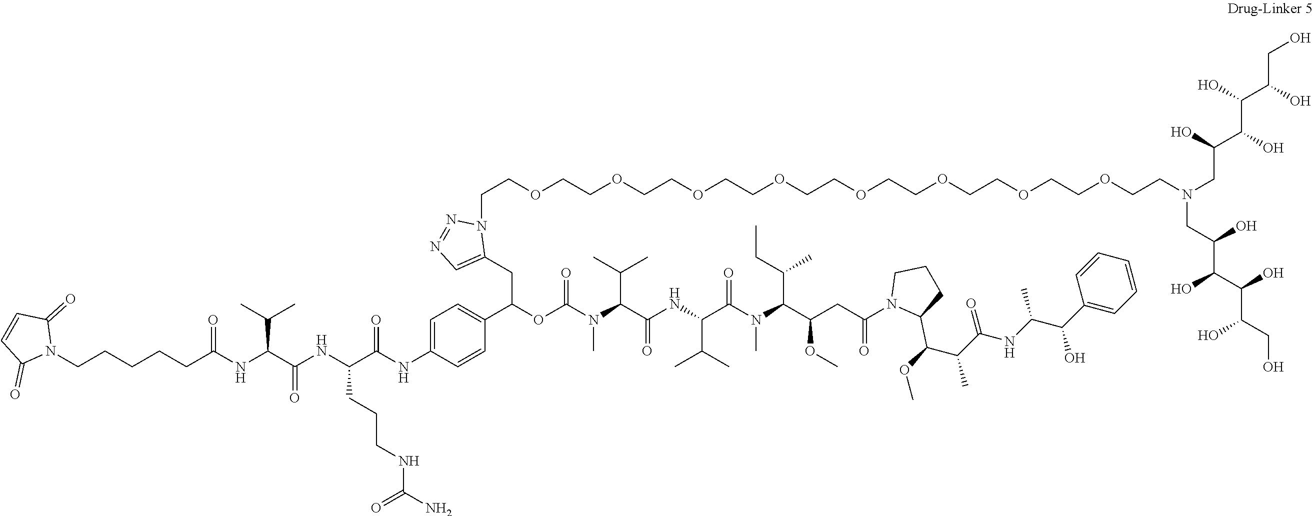 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

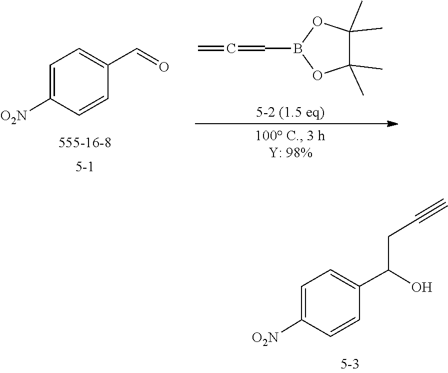

The mixture of 4-nitrobenzaldehyde 5-1 (10 g, 66.173 mmol) and 4,4,5,5-tetramethyl-2-(propa-1,2-dien-1-yl)-1,3,2-dioxaborolane 5-2 (17.532 mL, 99.259 mmol) was heated at 100° C. under nitrogen atmosphere with stirring for 3 h to achieve complete conversion. Then the resulting solution was purified by flash chromatography (silica gel, eluting with 0-30% EA in PE) to afford the product 5-3 (12.6 g, 65.903 mmol, 99.60%) as a pale yellow solid. ESI m/z: 192.1 (M+H)⁺.

### Step 2

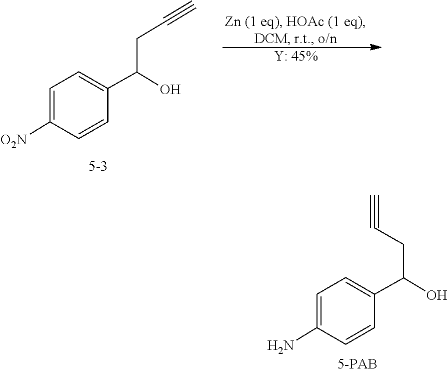

To the solution of 1-(4-nitrophenyl)but-3-yn-1-ol 5-3 (16 g, 83.686 mmol) in DCM (30 mL) was added Zn powder (0.767 mL, 83.686 mmol) and acetic acid (4.795 mL, 83.686 mmol). Then the mixture was stirred at room temperature for overnight. After completion, the mixture was filtered to remove the zinc solid, and the filtrate was concentrated and purified by reverse phase flash chromatography (C₁₈ column, eluting with 0-15% acetonitrile in water with 0.01% TFA) to afford the product 1-(4-aminophenyl)but-3-yn-1-ol 5-PAB (6.1 g, 37.841 mmol, 45.22%) as a brown solid. ESI m/z: 162.2 (M+H)⁺.

### Step 3

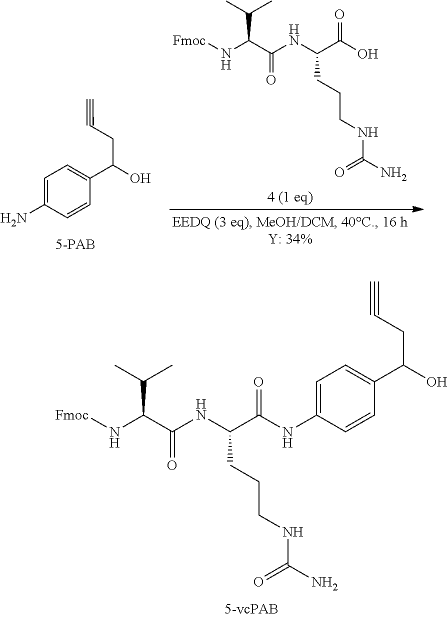

To the solution of (2S)-5-(carbamoylamino)-2-[(2S)-2-({[(9H-fluoren-9-yl)methoxy]carbonyl}amino)-3-methylbutanamido]pentanoic acid 4 (2.5 g, 5.035 mmol) in MeOH (8 mL) and DCM (32 mL) was added 1-(4-aminophenyl)but-3-yn-1-ol 5-PAB (0.81 g, 5.035 mmol) and EEDQ (3.74 g, 15.104 mmol). The mixture was stirred at 40° C. for overnight to complete. The resulting solution was concentrated to dryness and then purified by flash chromatography (silica gel, eluting with 0-10% methanol in DCM) to afford the product (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol, 32.30%) as a pale yellow solid. ESI m/z: 640.3 (M+H)⁺. ¹H NMR (400 MHz, DMSO-d6) δ 9.99 (s, 1H), 8.10 (d, J=7.6 Hz, 1H), 7.88 (d, J=7.6 Hz, 2H), 7.76-7.72 (m, 2H), 7.53 (d, J=8.8 Hz, 2H), 7.45-7.39 (m, 3H), 7.32-7.26 (m, 3H), 5.96 (m, 1H), 5.44-5.39 (m, 3H), 4.62 (t, J=6.0 Hz, 1H), 4.42-4.40 (m, 1H), 4.31-4.20 (m, 3H), 3.95-3.90 (m, 1H), 3.00-2.91 (m, 2H), 2.70-2.69 (m, 1H), 2.45-2.32 (m, 2H), 2.00-1.97 (m, 1H), 1.59-1.23 (m, 5H), 0.88-0.83 (m, 6H) ppm.

### Step 4

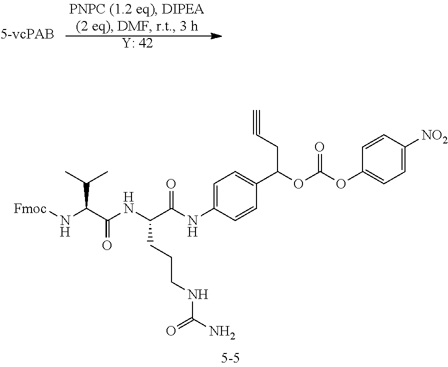

To the solution of (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol) and DIPEA (0.42 g, 3.252 mmol) in DMF (15 mL) was added bis(4-nitrophenyl) carbonate (PNPC, 0.59 g, 1.951 mmol) portionwise. After addition, the mixture was stirred at room temperature for another 3 h. After completion according to LCMS monitoring, the resulting solution was purified directly by reverse phase flash chromatography (C18 column, eluting with 0-75% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methyl butanamido]pentanamido]phenyl}but-3-yn-1-yl 4-nitrophenyl carbonate 5-5 (560 mg, 0.696 mmol, 42.75%) as a pink solid. ESI m/z: 805.3 (M+H)⁺.

### Step 5

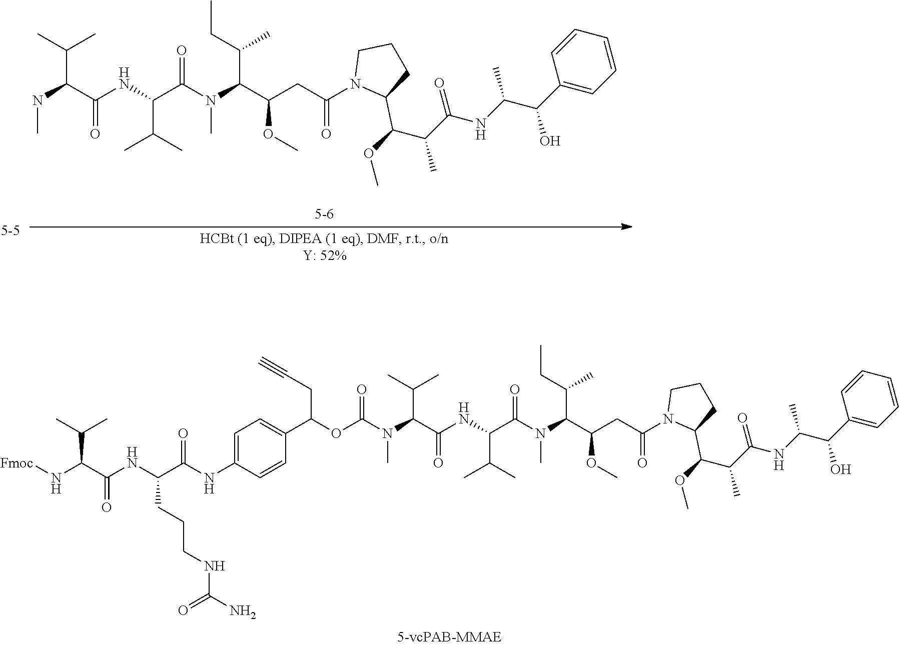

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl 4-nitrophenyl carbonate 5-5 (540 mg, 0.671 mmol) in DMF (15 mL) was added (2S)—N-[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]-3-methyl-2-(methylamino)butanamide 5-6 (481.72 mg, 0.671 mmol), HOBt (90.66 mg, 0.671 mmol) and DIPEA (86.71 mg, 0.671 mmol). The mixture was stirred at room temperature for overnight to achieve complete reaction. Then the resulting solution was purified directly by reverse phase flash chromatography (C8 column, eluting with 0-70% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (486 mg, 0.351 mmol, 52.34%) as a white solid. ESI m/z: 692.3 (M/2+H)⁺.

### Step 6

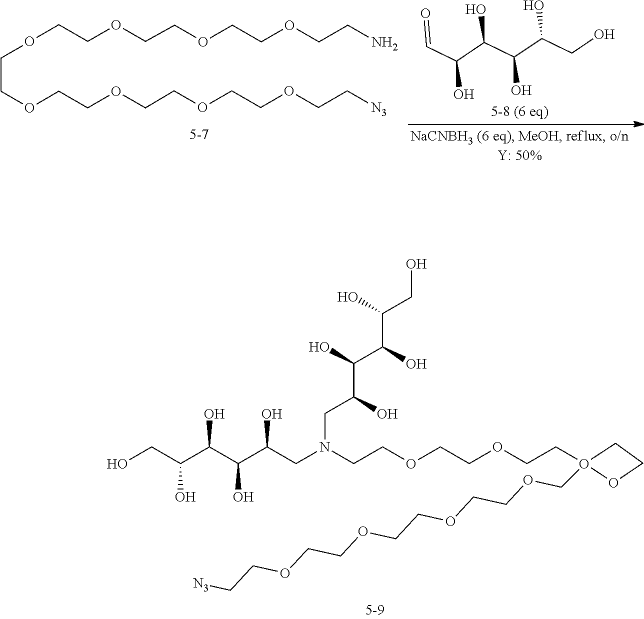

A solution of 26-azido-3,6,9,12,15,18,21,24-octaoxahexacosan-1-amine 5-7 (160 mg, 0.365 mmol) and D-glucose 5-8 (394.40 mg, 2.189 mmol) in MeOH (6 mL) was treated with NaBH₃CN (0.133 mL, 2.189 mmol). Then the mixture was stirred at 75° C. for overnight. Then the complete reaction solution was concentrated to dryness and the residue was purified by reverse phase flash chromatography (C8 column, eluting with 0-10% acetonitrile in water with 0.01% TFA) to afford the product (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (140 mg, 50%) as transparent oil. ESI m/z: 767.3 (M+H)⁺.

### Step 7

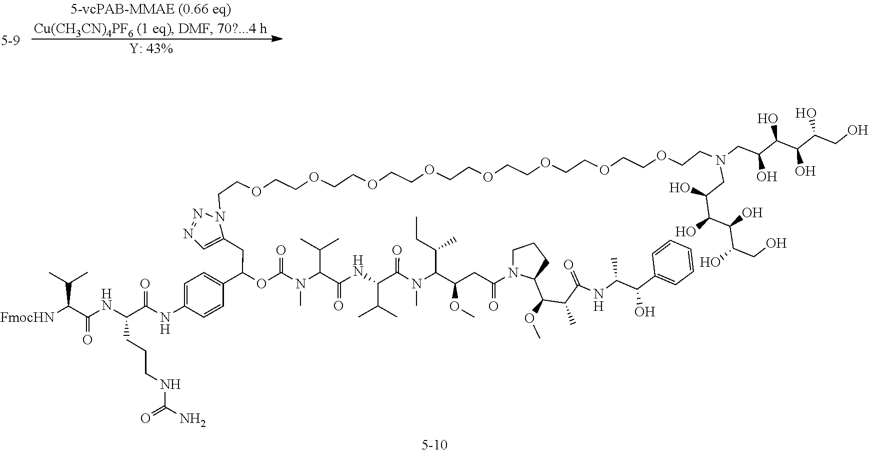

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (300 mg, 0.217 mmol) in DMF (6 mL) was added (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (249.38 mg, 0.325 mmol) and Cu(CH₃CN)₄PF₆ (121.55 mg, 0.325 mmol) at room temperature. The resulting mixture was heated at 70° C. with stirring for 4 h. The completed reaction solution was then purified by reverse phase flash chromatography (C8 column, eluting with 0-37% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31 R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (204 mg, 0.095 mmol, 43.71%) as a white solid. ESI m/z: 1075.6 (M/2+H)⁺.

### Step 8

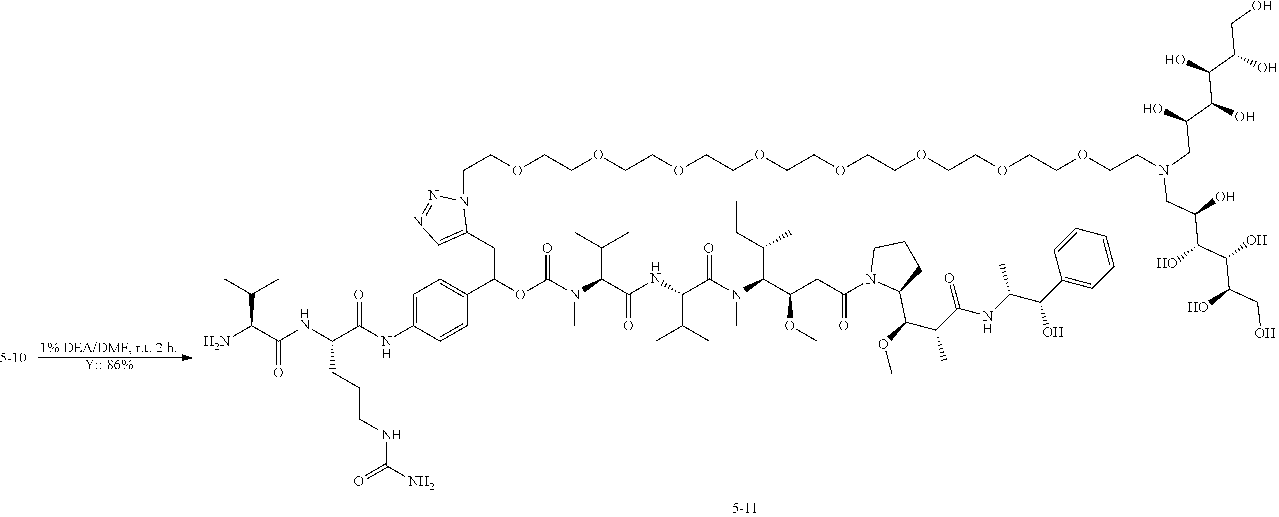 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@H](O)CO)c1ccc(NC(=O)[C@H](CCCNC(N)=O)NC(=O)[C@@H](N)C(C)C)cc1)C(C)C]

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31 R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (167 mg, 0.078 mmol) in DMF (2 mL) was added diethylamine (DEA, 0.02 mL, 0.125 mmol). The mixture was stirred at room temperature for 2 h to complete. The resulting solution was purified by reverse phase flash chromatography (C8 column, eluting with 0-40% acetonitrile in water with 0.01% TFA) to afford the product 1-{4-[(2S)-2-[(2S)-2-amino-3-methylbutanamido]-5-(carbamoylamino)pentanamido]phenyl}-2-{1-[(29S,30R,31 R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol, 86.81%) as a white solid. ESI m/z: 965.5 (M/2+H)⁺.

### Step 9

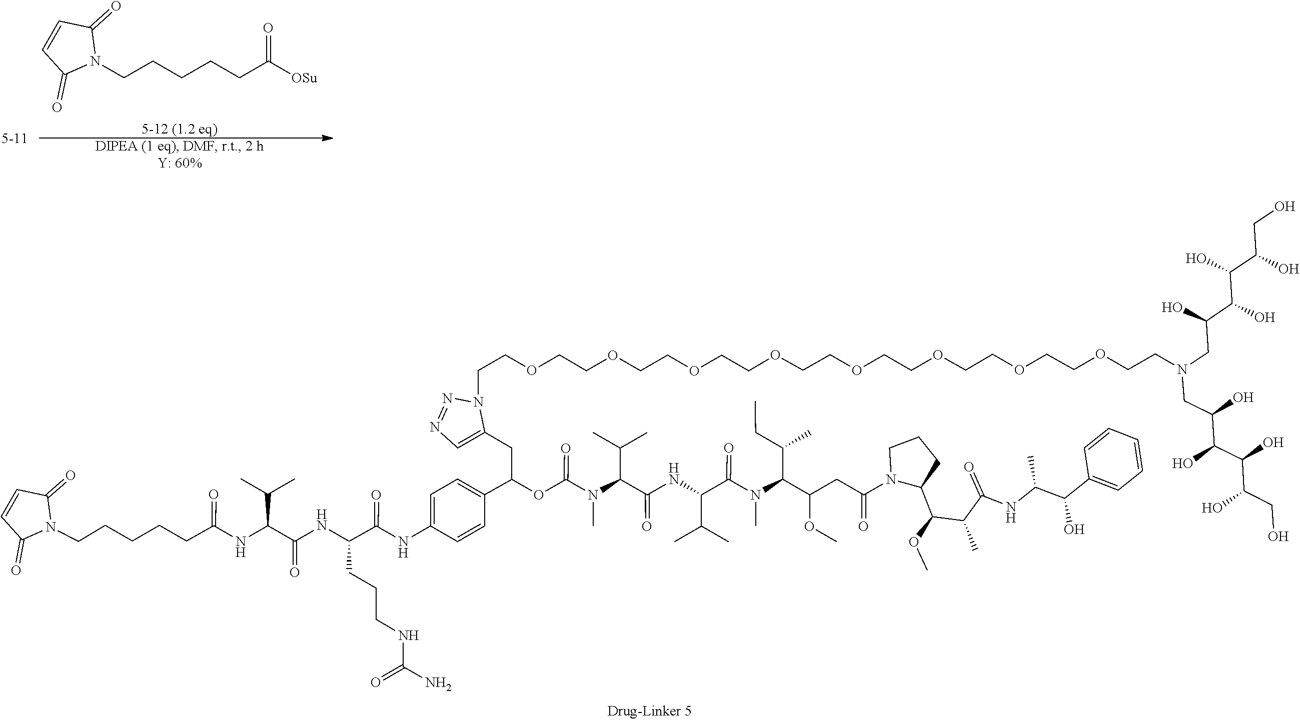

To the solution of 1-{4-[(2S)-2-[(2S)-2-amino-3-methylbutanamido]-5-(carbamoylamino)pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol) in DMF (3 mL) was added 2,5-dioxopyrrolidin-1-yl 6-(2,5-dioxo-2,5-dihydro-1H-pyrrol-1-yl)hexanoate 5-12(24.79 mg, 0.080 mmol) and DIPEA (8.66 mg, 0.067 mmol) sequentially. The resulting solution was stirred at room temperature for 2 h to complete full conversion. The reaction solution was then purified directly by reverse phase flash chromatography (C8 column, eluting with 0-40% acetonitrile in water with 001% TFA) to afford the product 1-{4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1 R,2R)-2-{[(1 R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (86 mg, 0.041 mmol, 60.13%) drug-linker 5 as a white solid. ESI m/z: 1061.8 (M/2+H)⁺, retention time 6.062 min (HPLC). ¹H NMR (400 MHz, DMSO-d6) δ 9.95 (s, 1H), 8.60-8.45 (m, 1H), 8.20-8.06 (m, 2H), 7.95-7.90 (m, 1H), 7.83-7.80 (m, 1H), 7.72-7.60 (m, 1.5H), 7.54-7.50 (m, 2H), 7.43-7.40 (m, 1H), 7.31-7.12 (m, 6H), 6.99 (s, 2H), 6.03 (brs, 1H), 5.82-5.68 (m, 1.5H), 5.50-5.30 (m, 3H), 4.76-4.52 (m, 3H), 4.50-4.30 (m, 8H), 4.29-4.11 (m, 3H), 4.00-3.92 (m, 8H), 3.77-3.66 (m, 16H), 3.66-3.48 (m, 18H), 3.45-3.29 (m, 12H), 3.24-3.07 (m, 12H), 3.00-2.68 (m, 8H), 2.44-1.94 (m, 7H), 1.80-1.67 (m, 4H), 1.58-1.34 (m, 10H), 1.21-1.15 (m, 2H), 1.05-0.96 (m, 7H), 0.90-0.50 (m, 28H) ppm.

### Example 6: Preparation of Drug-Linker 6

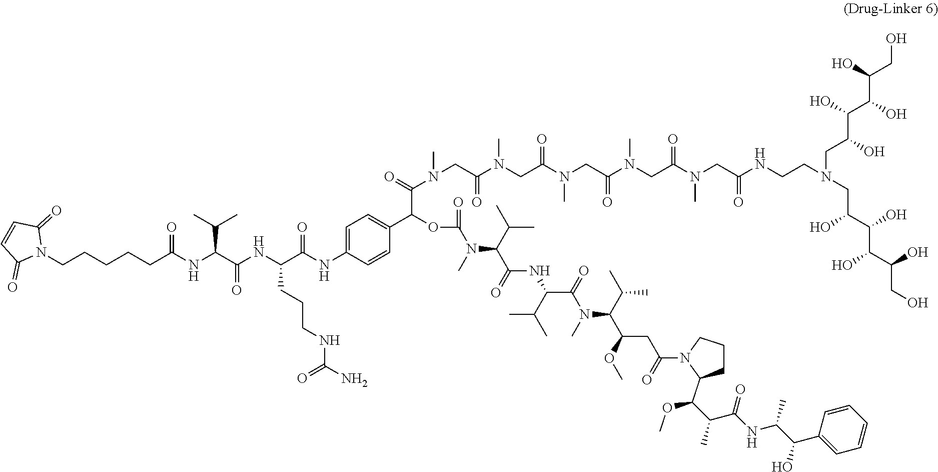

### Step 1

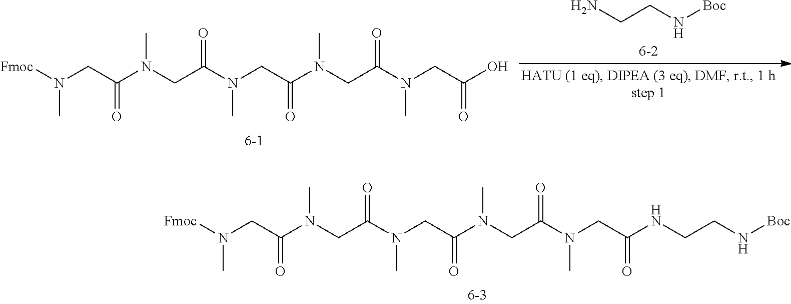

A solution of 6-1 (300 mg, 0.504 mmol), 6-2 (81 mg, 0.504 mmol) and HATU (192 mg, 0.504 mmol) in anhydrous DMF (10 mL) was stirred at room temperature for 5 min, then DIPEA (196 mg, 1.512 mmol) was added. The resulting solution was stirred for another 1 hr at r.t. until LCMS indicated complete reaction. The reaction solution was purified directly by reverse phase liquid chromatography to give 6-3 (227 mg, 0.308 mmol, 61.19%) as a white solid.

### Step 2

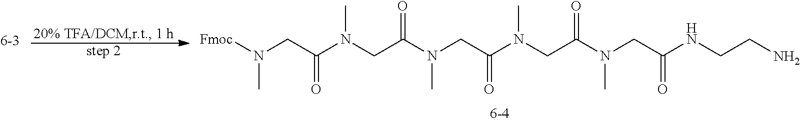

A solution of 6-3 (227 mg, 0.308 mmol) and TFA (2 mL) in anhydrous DCM (8 mL) was stirred at room temperature for 1 hr until LCMS indicated all starting amine was disappeared and desired product was detected. Then the solution was concentrated to dryness to 6-4 (185 mg, 0.290 mmol, 94.18%) as yellow oil, used as such in the next step.

### Step 3

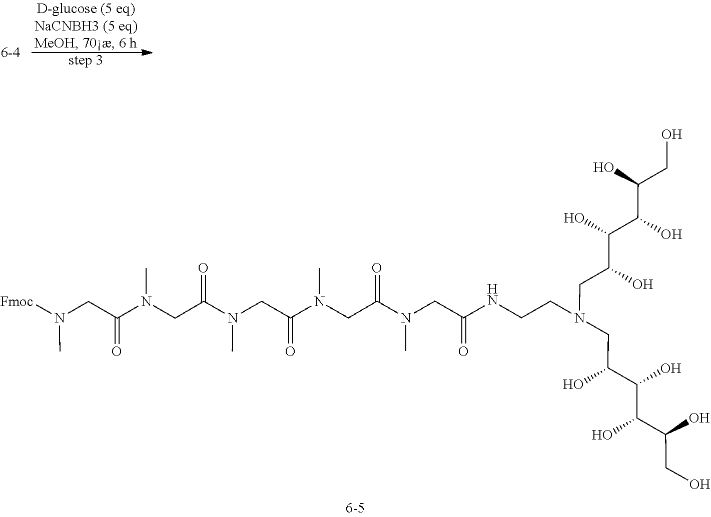

A solution of 6-4 (185 mg, 0.290 mmol) and D-Glucose (261 mg, 1.450 mmol) in anhydrous Methanol (50 mL) was heated at 50° C. for 30 min, then NaCNBH₃ (92 mg, 1.450 mmol) was added. The resulting solution was stirred for another 6 hr at 70° C. until indicated all starting amine was disappeared and the mass of desired product was detected. Then the reaction solution was concentrated and purified by reverse phase liquid chromatography to give 6-5 (125 mg, 0.129 mmol, 44.48%) as a white solid.

### Step 4

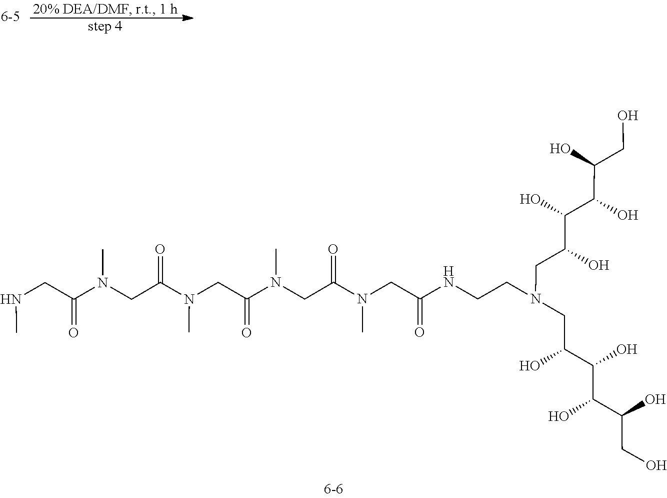 [SMILES: CNCC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 6-5 (125 mg, 0.129 mmol) and DEA (1 mL) in anhydrous DMF (4 mL) was stirred at room temperature for 1 hr until LCMS indicated all starting amine was disappeared and desired product was detected. Then the solution was concentrated to dryness to 6-6 (91 mg, 0.122 mmol, 94.57%) as colorless oil, used as such in the next step.

### Step 5

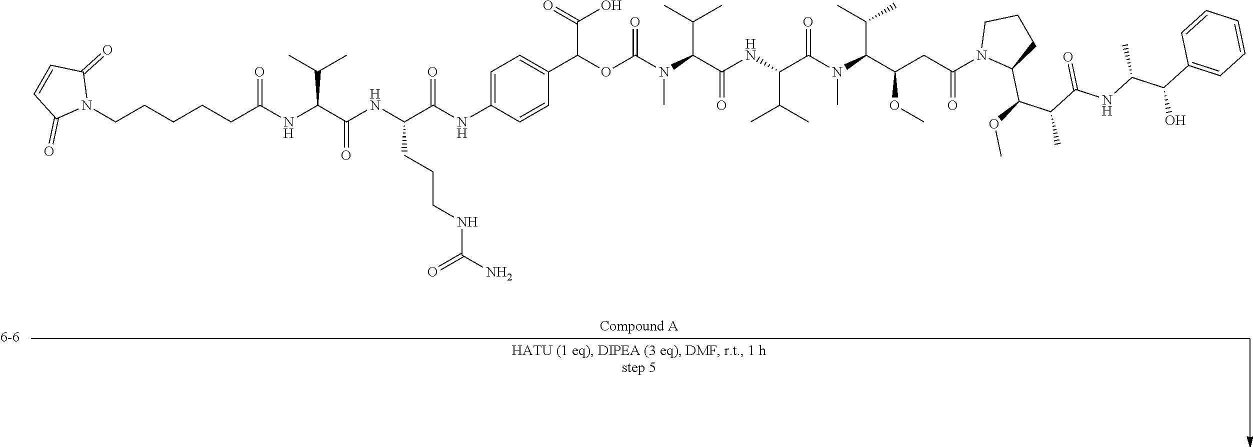

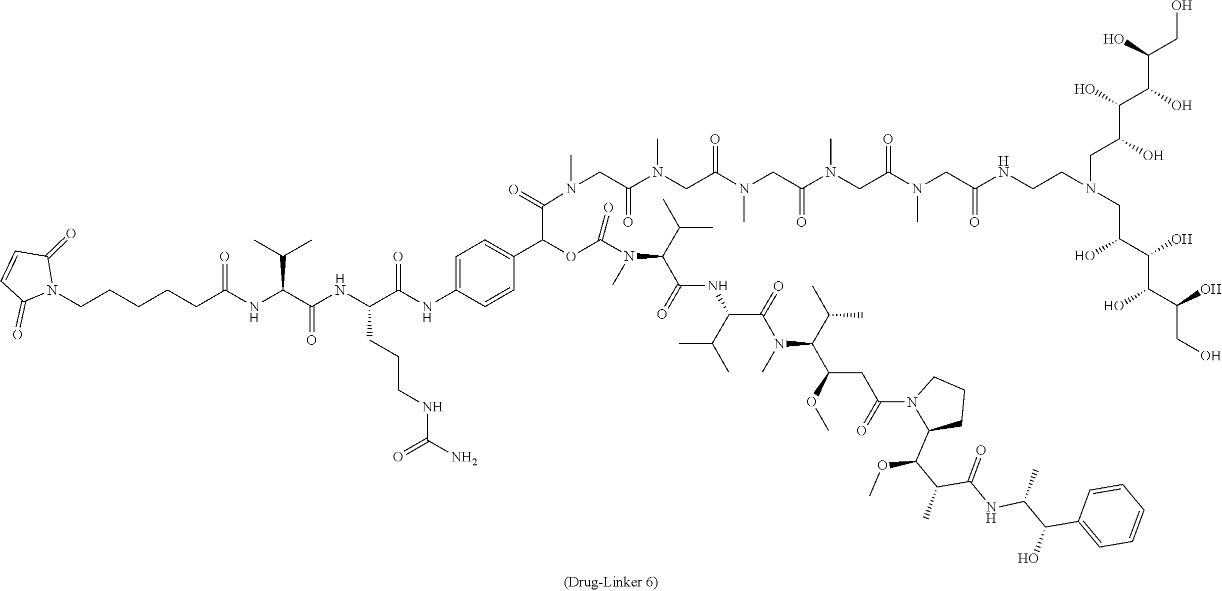

A solution of 6-6 (12 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give drug-linker 6 (12 mg, 0.006 mmol, 38.35%) as a white solid. LCMS, m/z=1044.15 (M/2+H)⁺; ¹H NMR (400 MHz, D₂O) δ 7.46-7.44 (m, 4H), 7.27-7.21 (m, 7H), 4.35-4.26 (m, 9H), 4.14-3.96 (m, 10H), 3.79-3.62 (m, 10H), 7.00 (s, 2H), 3.36-3.10 (m, 16H), 3.05-2.79 (m, 28H), 2.67-2.55 (m, 7H), 2.42-2.17 (m, 5H), 1.80-1.66 (m, 5H), 1.53-1.40 (m, 10H), 1.2-0.63 (m, 37H).

### Example 7: Preparation of Drug-Linker 7

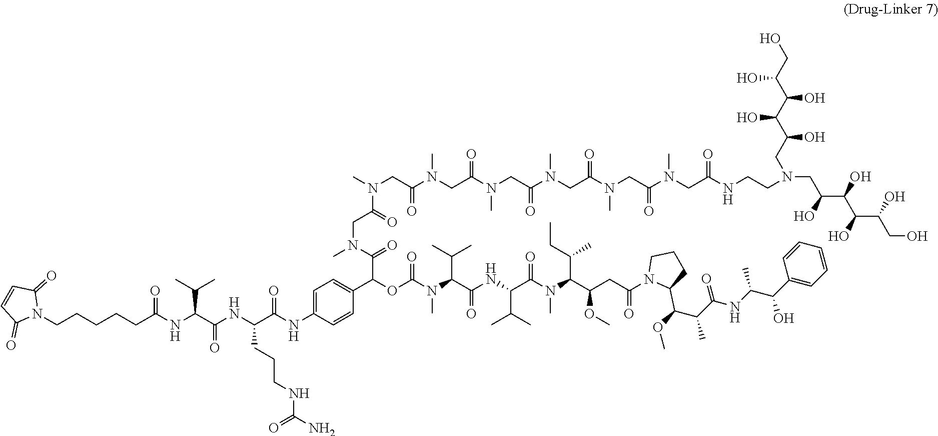 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

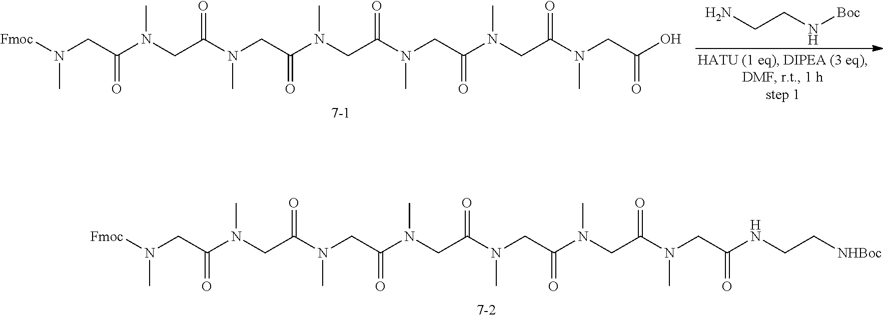

A solution of 7-1 (300 mg, 0.407 mmol), tert-butyl (2-aminoethyl)carbamate (65 mg, 0.407 mmol) and HATU (155 mg, 0.407 mmol) in anhydrous DMF (10 mL) was stirred at room temperature for 5 min, then DIPEA (158 mg, 1.221 mmol) was added.

The resulting solution was stirred for another 1 hr at r.t. until LCMS indicated complete reaction. The reaction solution was purified directly by reverse phase liquid chromatography to give 7-2 (243 mg, 0.276 mmol, 67.81%) as a white solid.

### Step 2

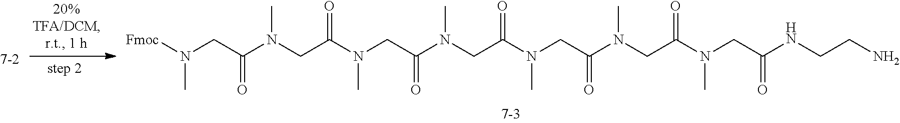

A solution of 7-2 (243 mg, 0.276 mmol) and TFA (2 mL) in anhydrous DCM (8 mL) was stirred at room temperature for 1 hr until LCMS indicated all starting amine was disappeared and desired product was detected. Then the solution was concentrated to dryness to 7-3 (207 mg, 0.265 mmol, 96.17%) as yellow oil, used as such in the next step.

### Step 3

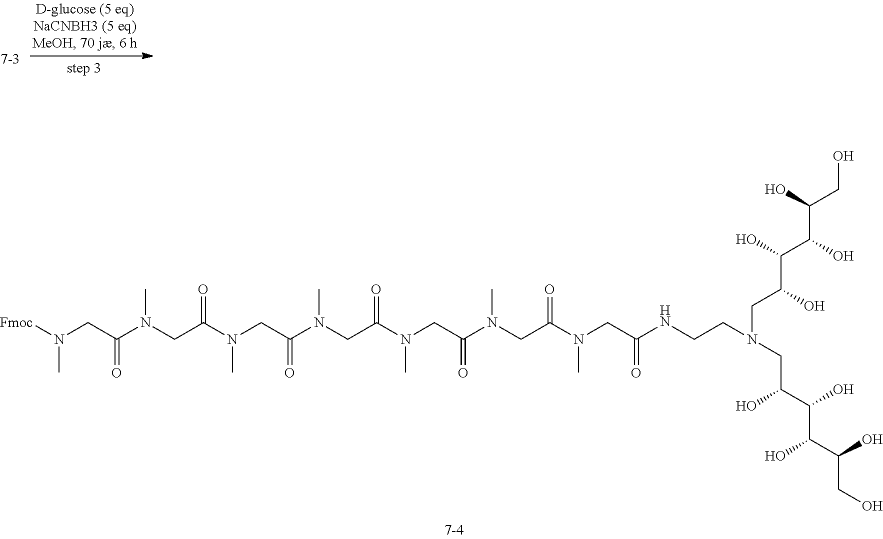

A solution of 7-3 (207 mg, 0.265 mmol) and D-Glucose (239 mg, 1.325 mmol) in anhydrous Methanol (50 mL) was heated at 50° C. for 30 min, then NaCNBH₃ (83 mg, 1.325 mmol) was added. The resulting solution was stirred for another 6 hr at 70° C. until indicated all starting amine was disappeared and the mass of desired product was detected. Then the reaction solution was concentrated and purified by reverse phase liquid chromatography to give 7-4 (147 mg, 0.133 mmol, 50.19%) as a white solid.

### Step 4

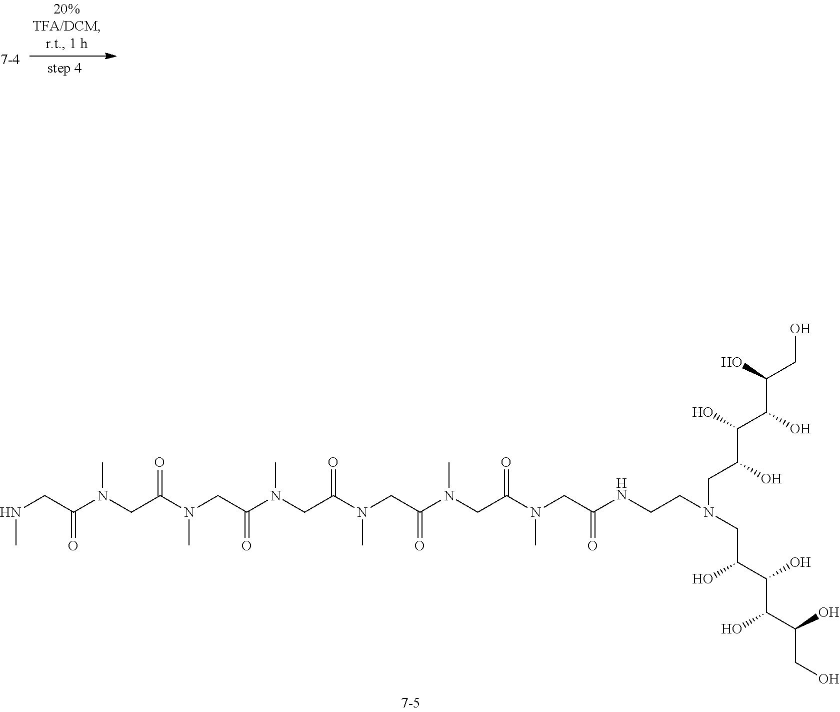 [SMILES: CNCC(=O)N(C)CC(=O)N(C)CC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 7-4 (147 mg, 0.133 mmol) and DEA (1 mL) in anhydrous DMF (4 mL) was stirred at room temperature for 1 hr until LCMS indicated all starting amine was disappeared and desired product was detected. Then the solution was concentrated to dryness to 7-5 (103 mg, 0.116 mmol, 87.41%) as colorless oil, used as such in the next step.

### Step 5

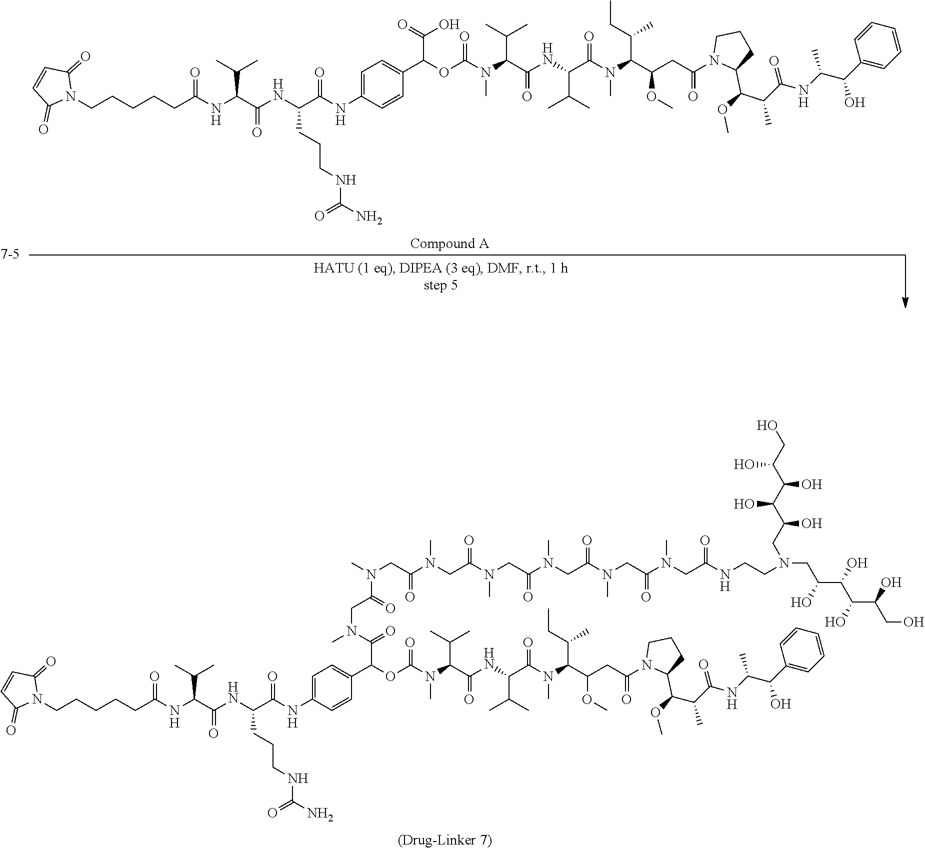

A solution of 7-5 (14 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give drug-linker 7 (15 mg, 0.007 mmol, 44.87%) as a white solid. LCMS, m/z=1115.24 (M/2+H)⁺.

### Example 8: Preparation of Drug-Linker 8

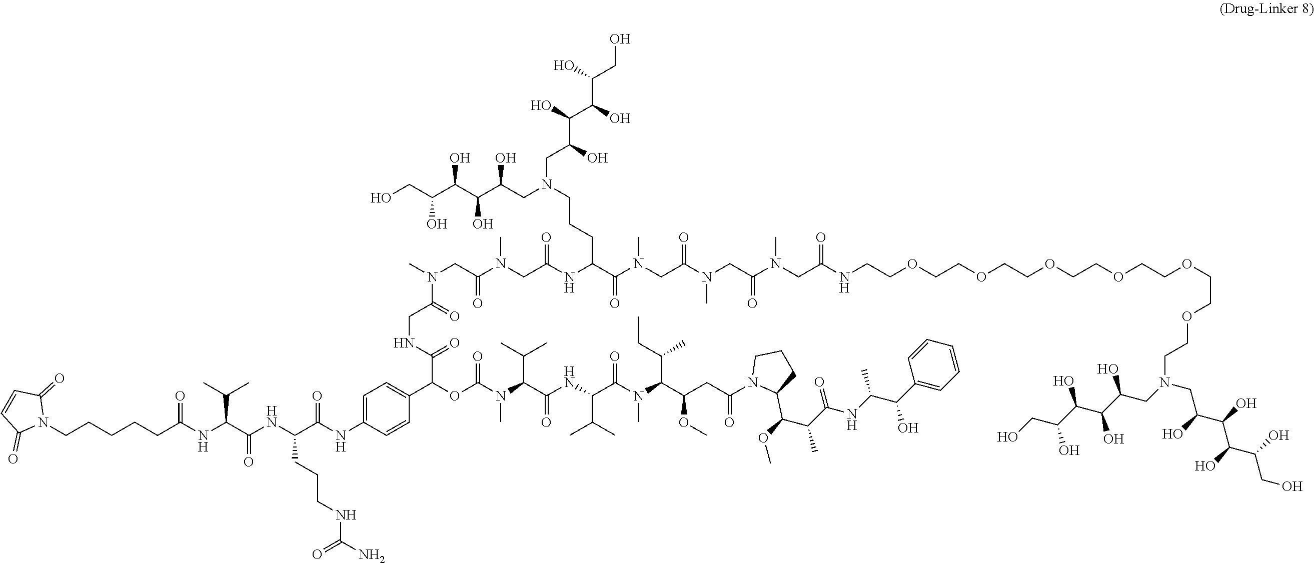 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

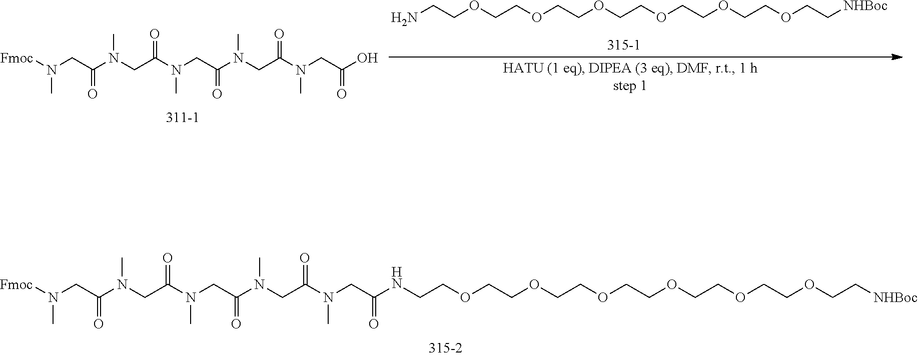

A solution of 8-1 (300 mg, 0.346 mmol), 8-2 (147 mg, 0.346 mmol) and HATU (132 mg, 0.346 mmol) in anhydrous DMF (5 mL) was stirred at room temperature for 5 min, then DIPEA (134 mg, 1.037 mmol) was added. The resulting solution was stirred for another 1 hr at r.t. until LCMS indicated complete reaction. The reaction solution was purified directly by reverse phase liquid chromatography to give 8-3 (310 mg, 0.243 mmol, 70.23%) as a white solid. Purity, 95%.

### Step 2

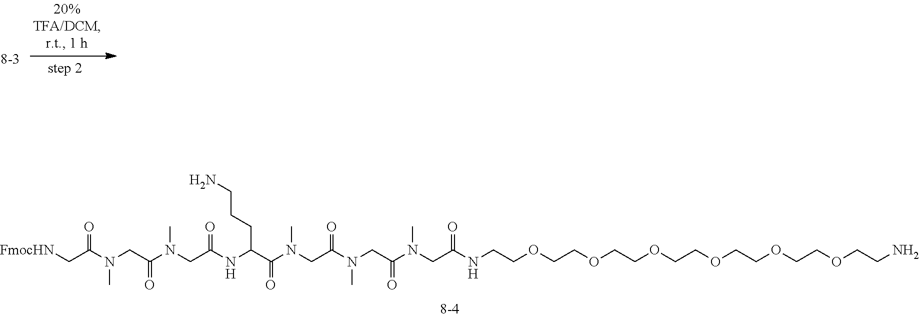

A solution of 8-3 (310 mg, 0.243 mmol) and TFA (1 mL) in anhydrous DCM (4 mL) was stirred at room temperature for 1 hr until LCMS indicated all starting amine was disappeared and desired product was detected. Then the solution was concentrated to dryness to 8-4 (255 mg, 0.238 mmol, 97.78%) as yellow oil, used as such in the next step. Purity, 95%.

### Step 3

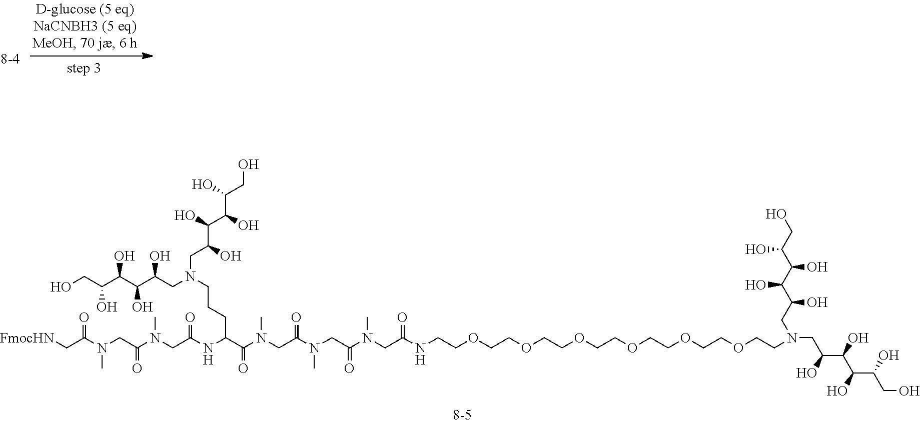

A solution of 8-4 (255 mg, 0.238 mmol) and D-Glucose (428 mg, 2.376 mmol) in anhydrous Methanol (50 mL) was heated at 50° C. for 30 min, then NaCNBH₃ (150 mg, 2.387 mmol) was added. The resulting solution was stirred for another 16 hr at 70° C. until indicated all starting amine was disappeared and the mass of desired product was detected. Then the reaction solution was concentrated and purified by reverse phase liquid chromatography to give 8-5 (185 mg, 0.107 mmol, 44.96%) as a white solid. Purity, 95%.

### Step 4

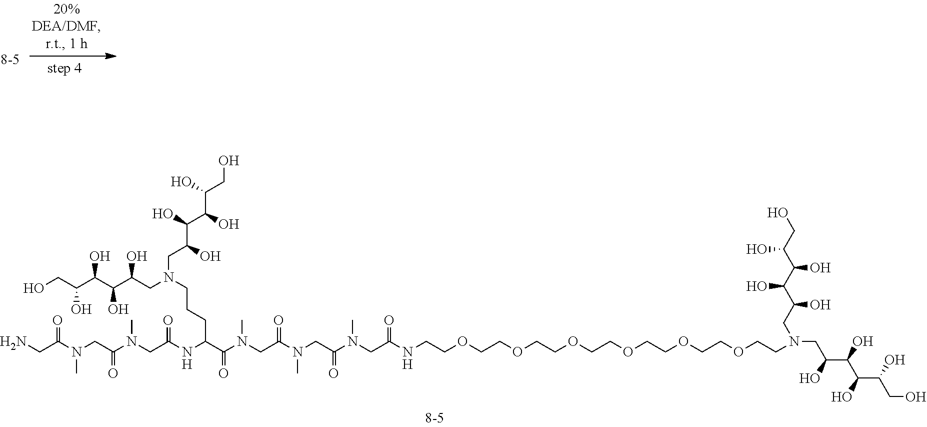 [SMILES: CN(CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 8-5 (185 mg, 0.107 mmol) and DEA (1 mL) in anhydrous DMF (4 mL) was stirred at room temperature for 1 hr until LCMS indicated all starting amine was disappeared and desired product was detected. Then the solution was concentrated to dryness to 8-6 (155 mg, 0.103 mmol, 96.08%) as colorless oil, used as such in the next step. Purity, 95%.

### Step 5

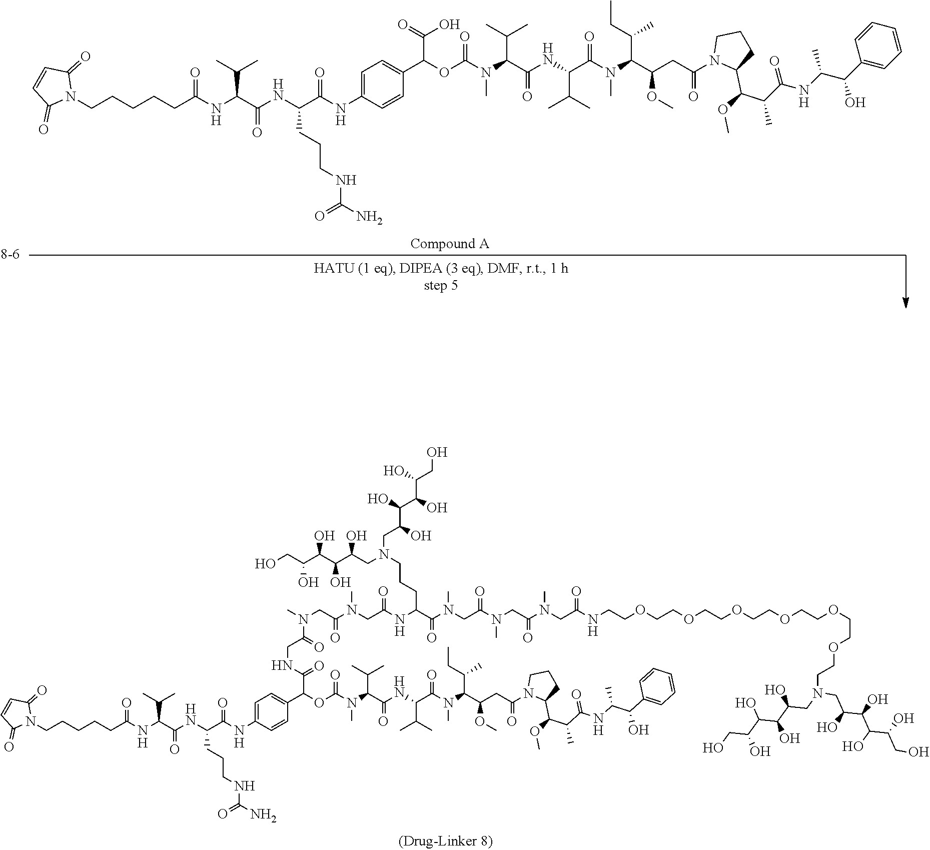

A solution of 8-6 (100 mg, 0.066 mmol), Compound A (90 mg, 0.066 mmol) and HATU (25 mg, 0.066 mmol) in anhydrous DMF (4 mL) was stirred at room temperature for 5 min, then DIPEA (26 mg, 0.201 mmol) was added. The resulting solution was stirred for another 1 hr at r.t. until LCMS indicated complete reaction. Then the reaction solution was purified by prep. HPLC to give drug-linker 8 (10 mg, 0.004 mmol, 5.32%) as a white solid. LCMS, m/z=1425.93 (M/2+H)⁺.

### Example 9: Preparation of Drug-Linker 9

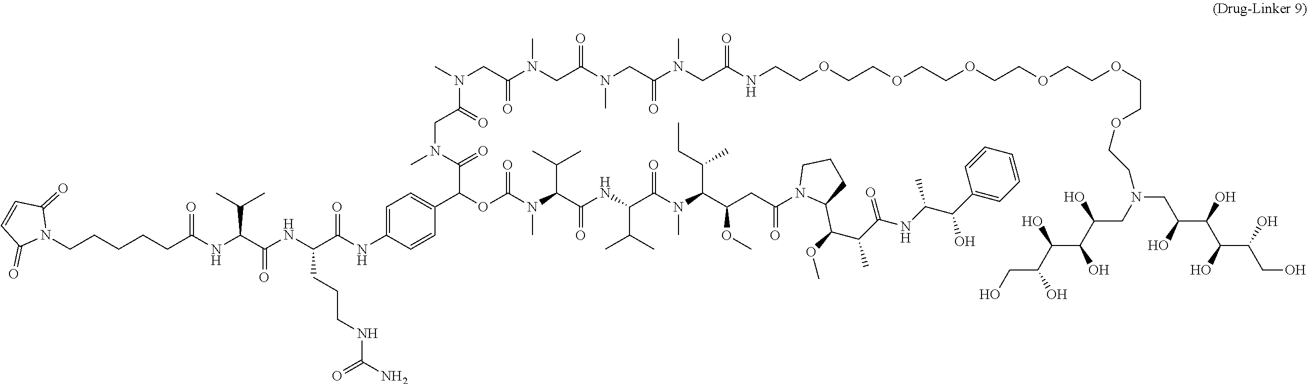 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

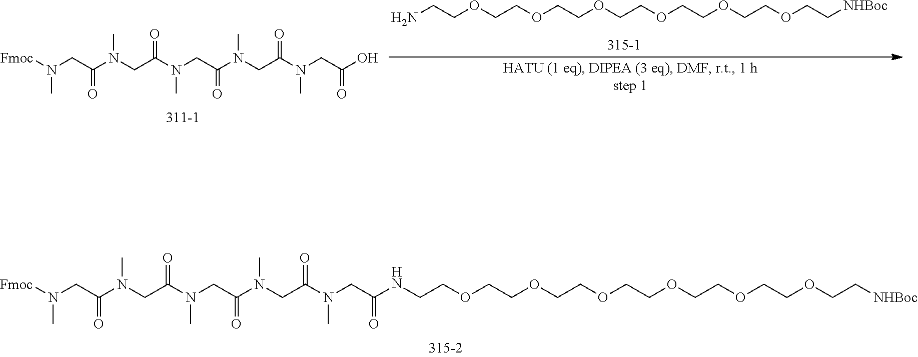

A solution of 311-1 (300 mg, 0.504 mmol), 315-1 (213 mg, 0.504 mmol) and HATU (192 mg, 0.407 mmol) in anhydrous DMF (10 mL) was stirred at room temperature for 5 min, then DIPEA (196 mg, 1.517 mmol) was added. The resulting solution was stirred for another 1 hr at r.t. until LCMS indicated complete reaction. The reaction solution was purified directly by reverse phase liquid chromatography to give 315-2 (315 mg, 0.314 mmol, 62.30%) as a white solid.

### Step 2

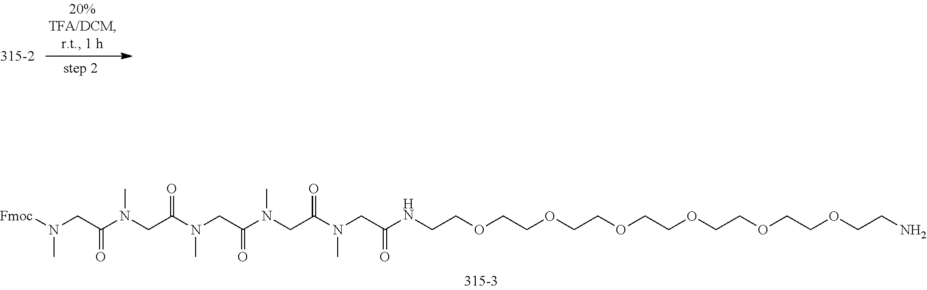

A solution of 315-2 (315 mg, 0.314 mmol) and TFA (2 mL) in anhydrous DCM (8 mL) was stirred at room temperature for 1 hr until LCMS indicated all starting amine was disappeared and desired product was detected. Then the solution was concentrated to dryness to 315-3 (264 mg, 0.293 mmol, 93.31%) as yellow oil, used as such in the next step.

### Step 3

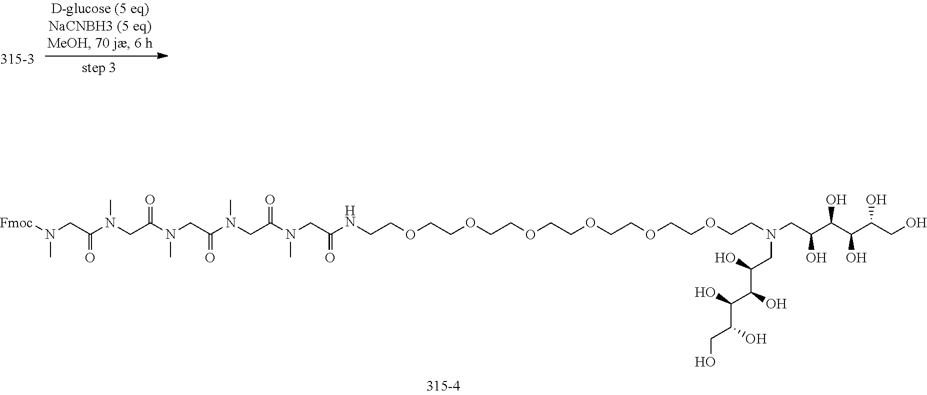

A solution of 315-3 (264 mg, 0.293 mmol) and D-Glucose (263 mg, 1.461 mmol) in anhydrous Methanol (50 mL) was heated at 50° C. for 30 min, then NaCNBH₃ (92 mg, 1.464 mmol) was added. The resulting solution was stirred for another 6 hr at 70° C. until indicated all starting amine was disappeared and the mass of desired product was detected. Then the reaction solution was concentrated and purified by reverse phase liquid chromatography to give 315-4 (233 mg, 0.189 mmol, 64.51%) as a white solid.

### Step 4

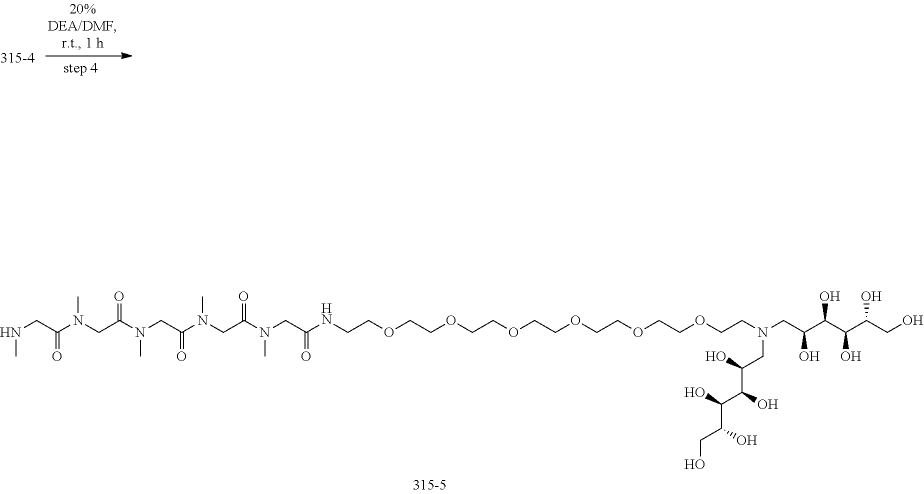 [SMILES: CNCC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-4 (233 mg, 0.189 mmol) and DEA (1 mL) in anhydrous DMF (4 mL) was stirred at room temperature for 1 hr until LCMS indicated all starting amine was disappeared and desired product was detected. Then the solution was concentrated to dryness to 315-5 (177 mg, 0.176 mmol, 93.12%) as colorless oil, used as such in the next step.

### Step 5

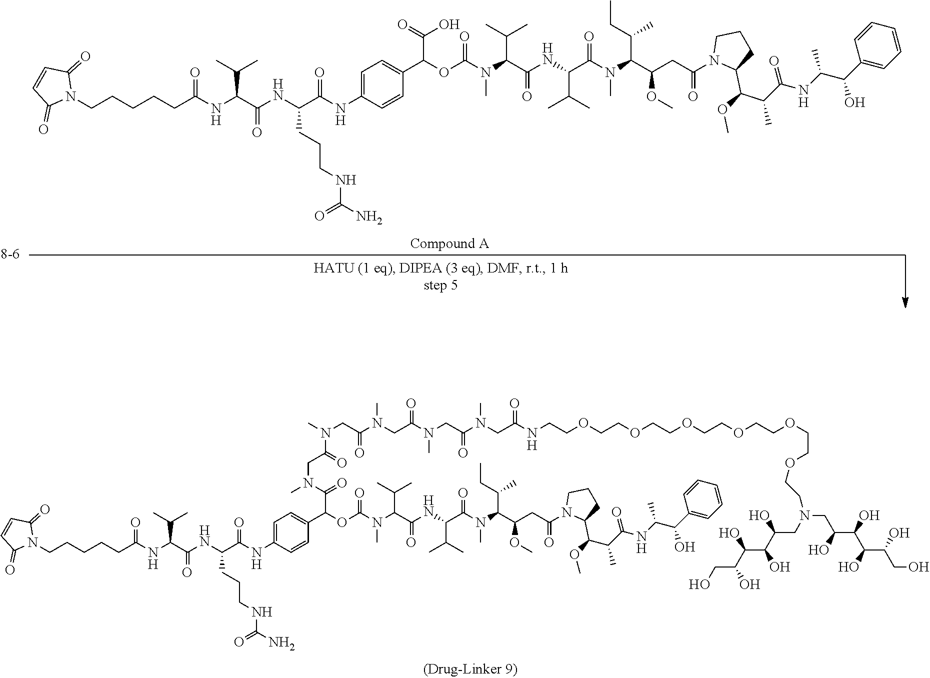

A solution of 315-5 (30 mg, 0.030 mmol), Compound A (40 mg, 0.030 mmol) and HATU (11 mg, 0.030 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 8 (18 mg, 0.007 mmol, 25.53%) as a white solid. LCMS, m/z=1176.02 (M/2+H)⁺.

### Example 10: Preparation of Drug-Linker 10

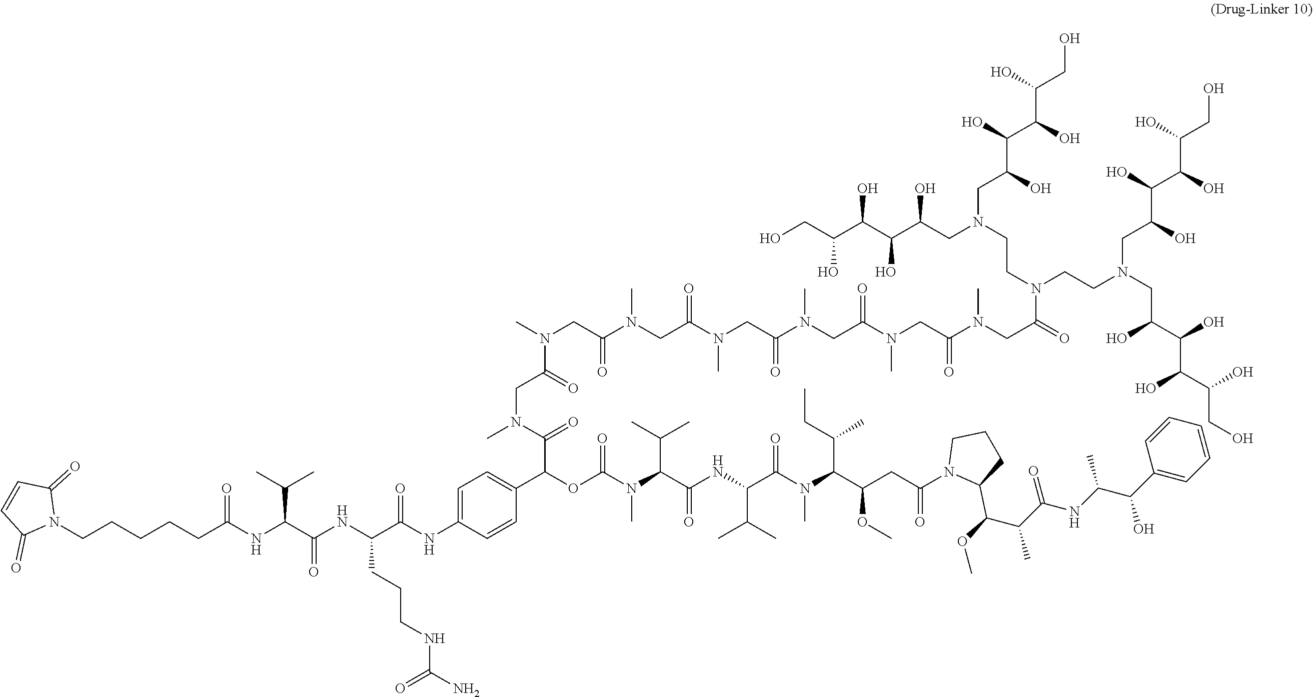 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

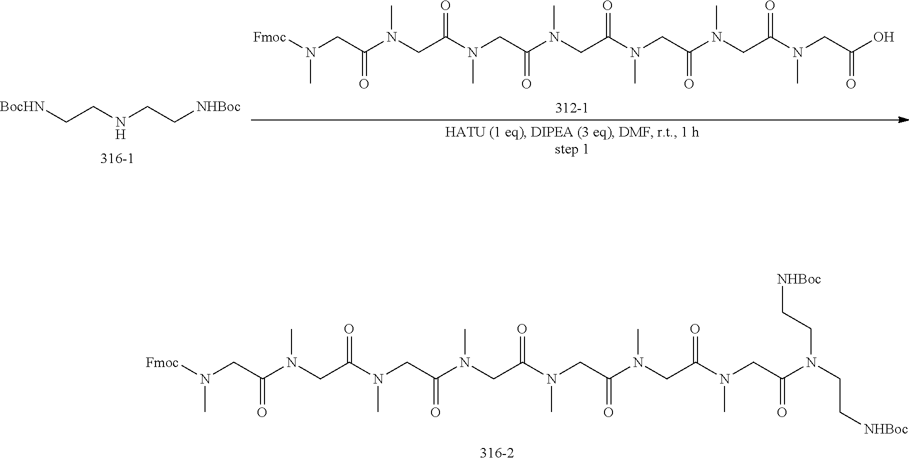

A solution of 316-1 (82 mg, 0.270 mmol), 312-1 (200 mg, 0.270 mmol) and HATU (103 mg, 0.270 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 316-2 (205 mg, 0.200 mmol, 74.07%) as a white solid.

### Step 2

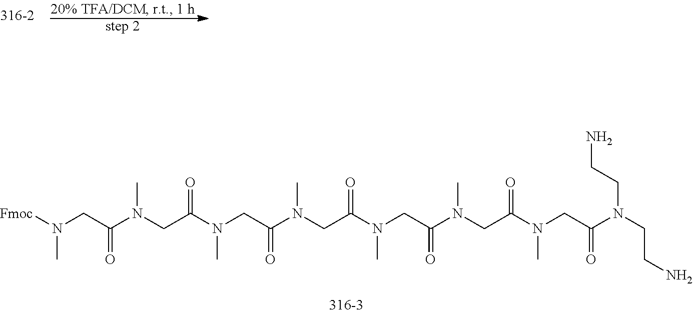

A solution of 316-2 (205 mg, 0.200 mmol) and TFA (1 mL) in anhydrous DCM (4 mL) was stirred at room temperature for 1 hr until LCMS indicated all starting amine was disappeared and desired product was detected. Then the solution was concentrated to dryness to 316-3 (157 mg, 0.191 mmol, 93.17%) as yellow oil, used as such in the next step.

### Step 3

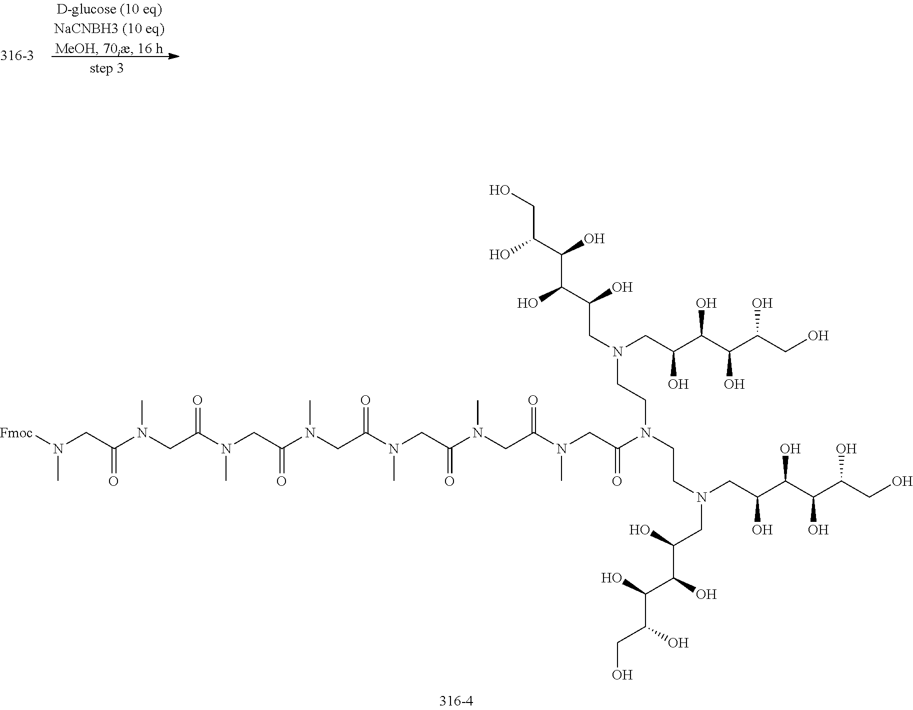

A solution of 316-3 (157 mg, 0.191 mmol) and D-Glucose (172 mg, 0.955 mmol) in anhydrous Methanol (50 mL) was heated at 50° C. for 30 min, then NaCNBH₃ (60 mg, 0.955 mmol) was added. The resulting solution was stirred for another 16 hr at 70° C. until indicated all starting amine was disappeared and the mass of desired product was detected. Then the reaction solution was concentrated and purified by reverse phase liquid chromatography to give 316-4 (135 mg, 0.091 mmol, 47.77%) as a white solid.

### Step 4

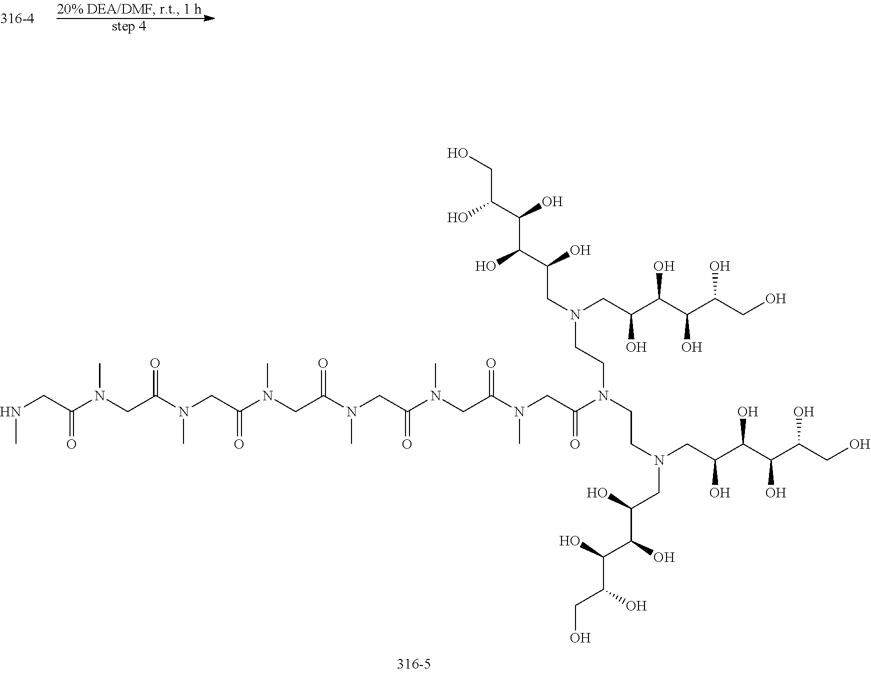 [SMILES: CNCC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO]

A solution of 316-4 (135 mg, 0.091 mmol) and DEA (1 mL) in anhydrous DMF (4 mL) was stirred at room temperature for 1 hr until LCMS indicated all starting amine was disappeared and desired product was detected. Then the solution was concentrated to dryness to 316-5 (114 mg, 0.090 mmol, 98.90%) as colorless oil, used as such in the next step.

### Step 5

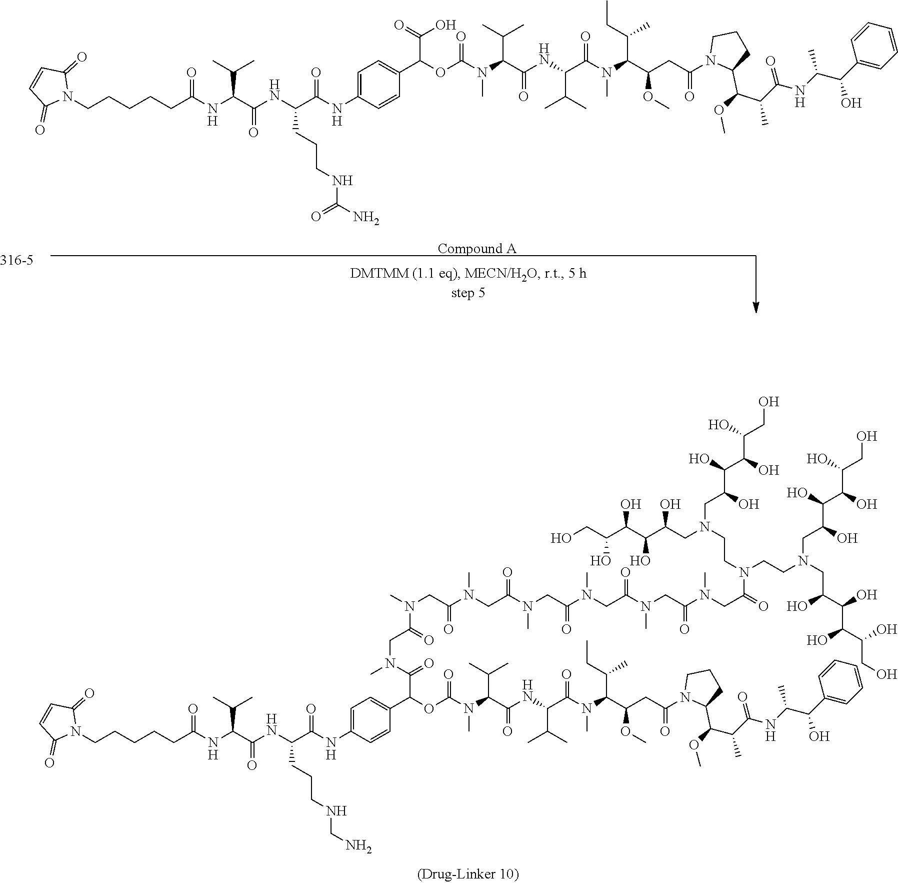

A solution of 316-5 (100 mg, 0.080 mmol), Compound A (108 mg, 0.080 mmol) and DMTMM (24 mg, 0.088 mmol) in H₂O (4 mL) and MeCN (4 mL) was stirred at room temperature for 5 h. Then the reaction solution was purified by prep. HPLC to give drug-linker 10 (7 mg, 0.003 mmol, 3.37%) as a white solid. LCMS, m/z=1300.87 (M/2+H)⁺.

### Example 11: Preparation of Drug-Linker 11

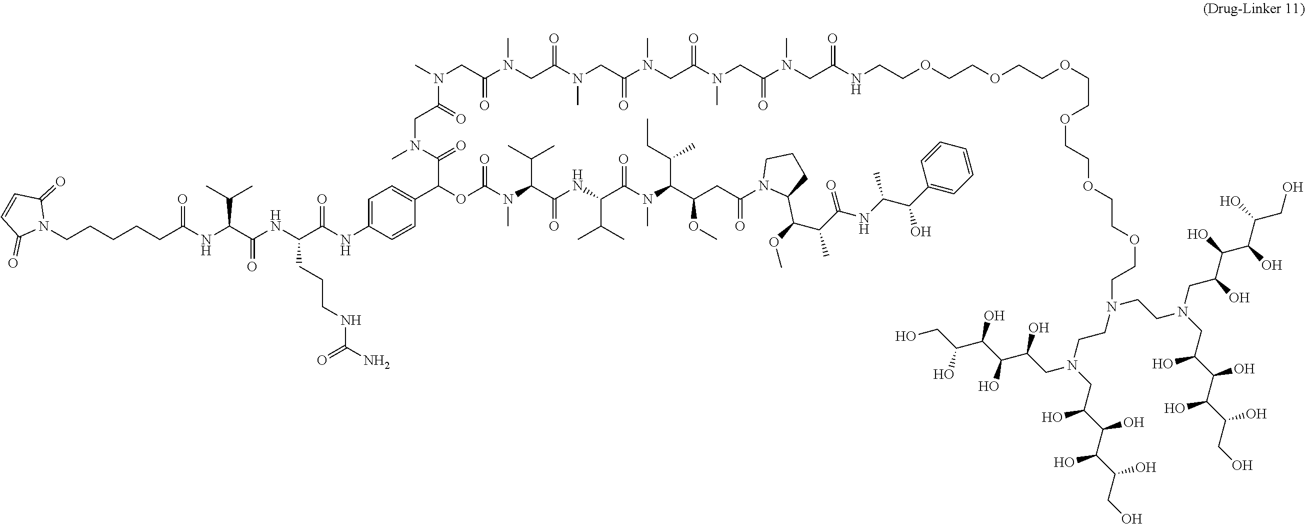 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

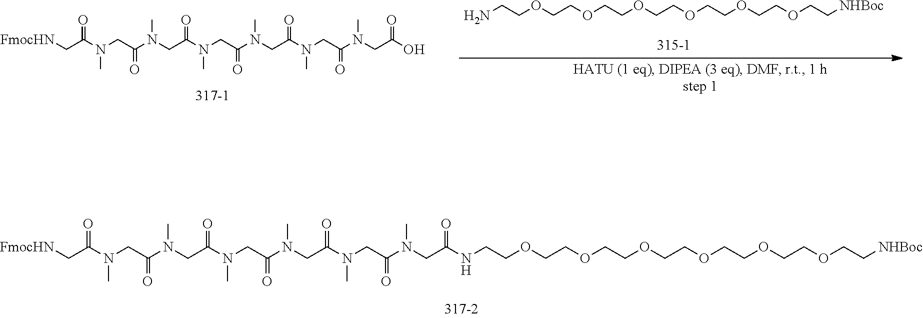

A solution of 317-1 (400 mg, 0.553 mmol), 315-1 (235 mg, 0.554 mmol) and HATU (210 mg, 0.553 mmol) in anhydrous DMF (6 mL) was stirred at room temperature for 5 min, then DIPEA (214 mg, 1.656 mmol) was added. The resulting solution was stirred for another 1 hr at room temperature until LCMS indicated complete reaction.

The reaction solution was purified directly by reverse phase liquid chromatography to give 317-2 (410 mg, 0.363 mmol, 65.59%) as a white solid.

### Step 2

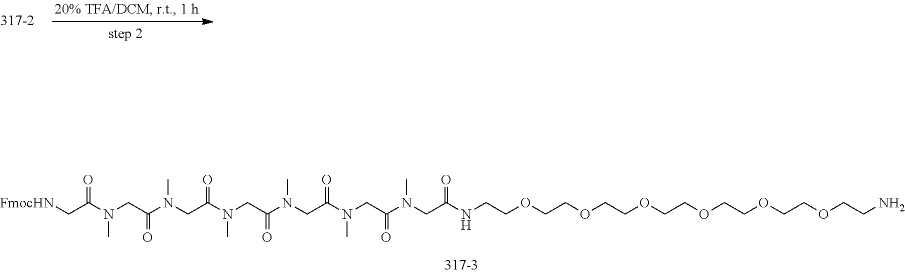

A solution of 317-2 (410 mg, 0.363 mmol) and TFA (2 mL) in anhydrous DCM (8 mL) was stirred at room temperature for 1 hr until LCMS indicated all starting amine was disappeared and desired product was detected. Then the solution was concentrated to dryness to 317-3 (365 mg, 0.354 mmol, 97.60%) as yellow oil, used as such in the next step.

### Step 3

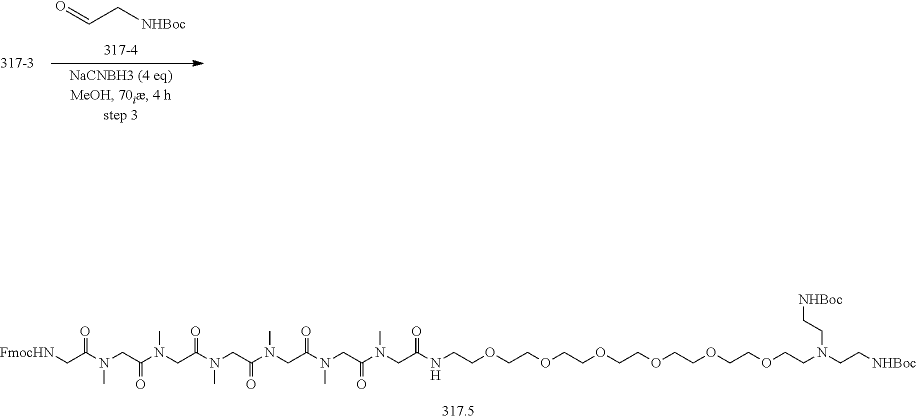

A solution of 317-3 (365 mg, 0.354 mmol) and 317-4 (338 mg, 2.123 mmol) in anhydrous Methanol (50 mL) was heated at 50° C. for 30 min, then NaCNBH₃ (134 mg, 2.132 mmol) was added. The resulting solution was stirred for another 4 hr at 70° C. until indicated all starting amine was disappeared and the mass of desired product was detected. Then the reaction solution was concentrated and purified by reverse phase liquid chromatography to give 317-5 (310 mg, 0.235 mmol, 66.51%) as a white solid.

### Step 4

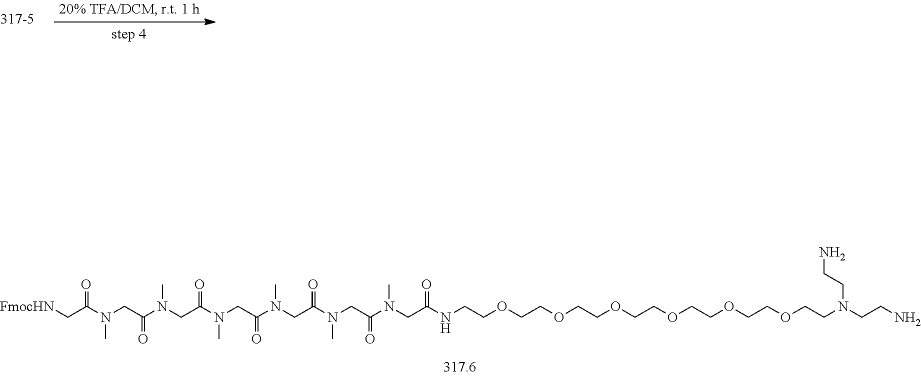

A solution of 317-5 (310 mg, 0.235 mmol) and TFA (2 mL) in anhydrous DCM (8 mL) was stirred at room temperature for 1 hr until LCMS indicated all starting amine was disappeared and desired product was detected. Then the solution was concentrated to dryness to 317-6 (250 mg, 0.224 mmol, 95.30%) as yellow oil, used as such in the next step.

### Step 5

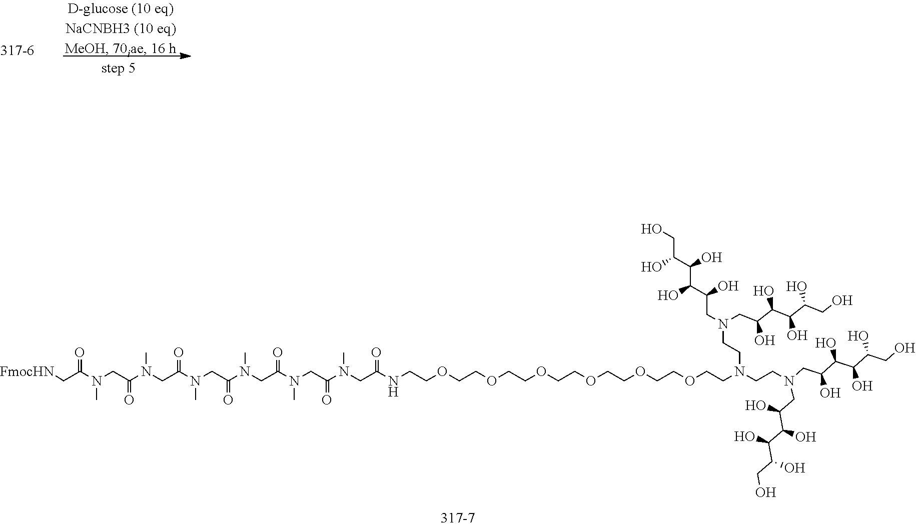

A solution of 317-6 (250 mg, 0.224 mmol) and D-Glucose (484 mg, 2.687 mmol) in anhydrous Methanol (50 mL) was heated at 50° C. for 30 min, then NaCNBH₃ (169 mg, 2.689 mmol) was added. The resulting solution was stirred for another 48 hr at 70° C. until indicated all starting amine was disappeared and the mass of desired product was detected. Then the reaction solution was concentrated and purified by reverse phase liquid chromatography to give 317-7 (174 mg, 0.098 mmol, 43.81%) as a white solid.

### Step 6

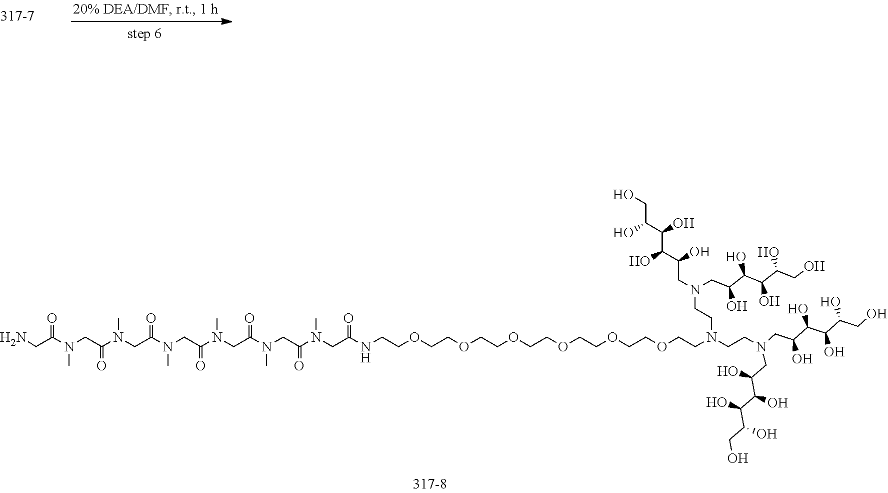 [SMILES: CN(CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)CN(C)C(=O)CN(C)C(=O)CN(C)C(=O)CN]

A solution of 317-7 (174 mg, 0.098 mmol) and DEA (1 mL) in anhydrous DMF (4 mL) was stirred at room temperature for 1 hr until LCMS indicated all starting amine was disappeared and desired product was detected. Then the solution was concentrated to dryness to 317-8 (145 mg, 0.094 mmol, 95.41%) as colorless oil, used as such in the next step.

### Step 7

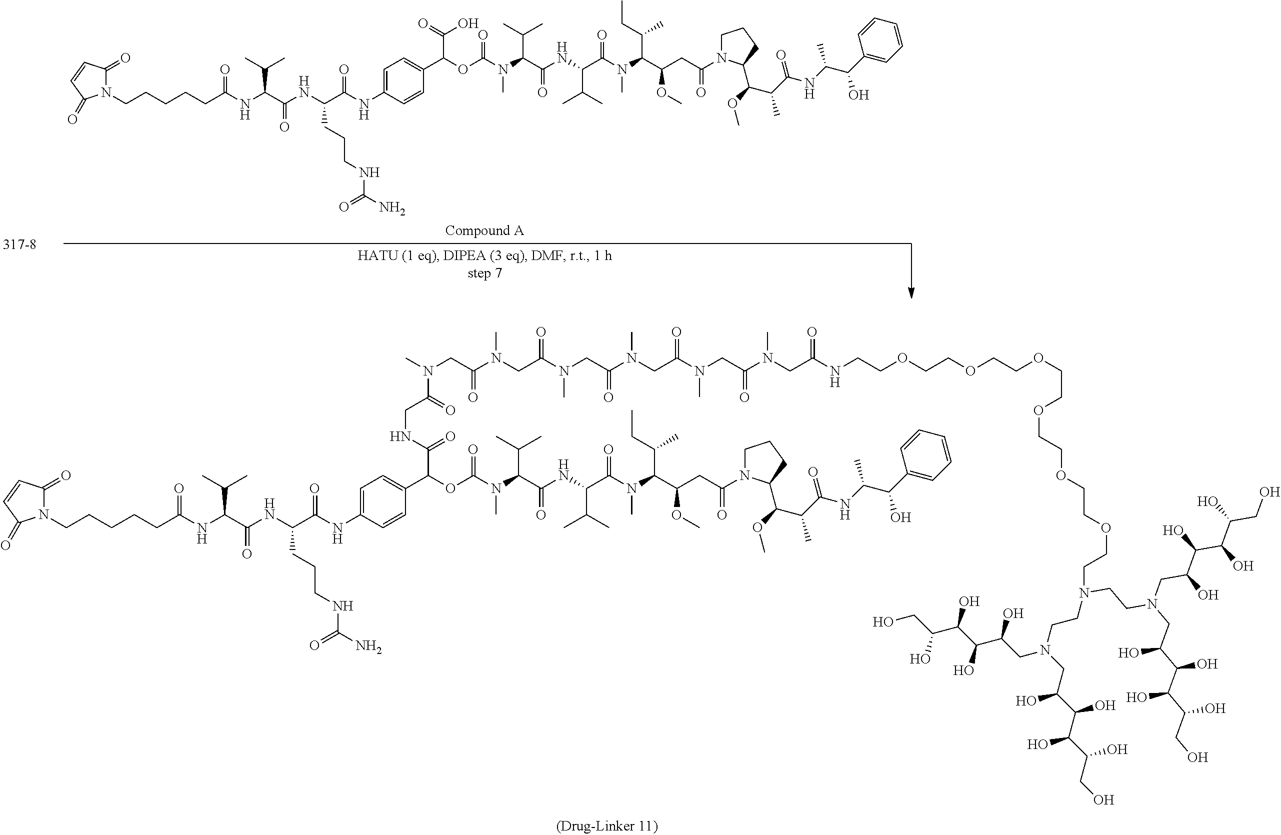

A solution of 317-8 (80 mg, 0.052 mmol), Compound A (70 mg, 0.052 mmol) and HATU (20 mg, 0.052 mmol) in anhydrous DMF (4 mL) was stirred at room temperature for 5 min, then DIPEA (20 mg, 0.155 mmol) was added. The resulting solution was stirred for another 1 hr at r.t. until LCMS indicated complete reaction. Then the reaction solution was purified by prep. HPLC to give drug-linker 11 (40 mg, 0.014 mmol, 26.59%) as a white solid. LCMS, m/z=1447.20 (M/2+H)⁺.

### Example 12: Preparation of Drug-Linker 12

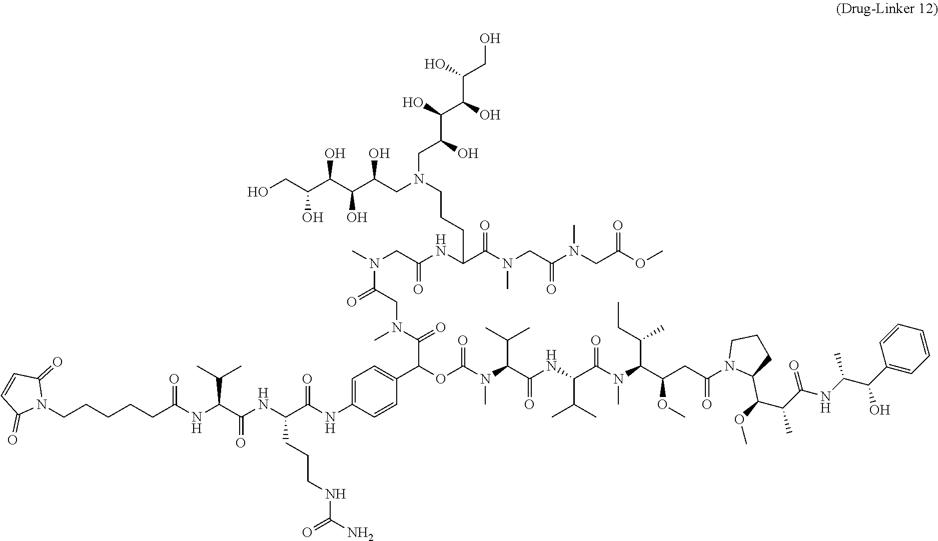 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

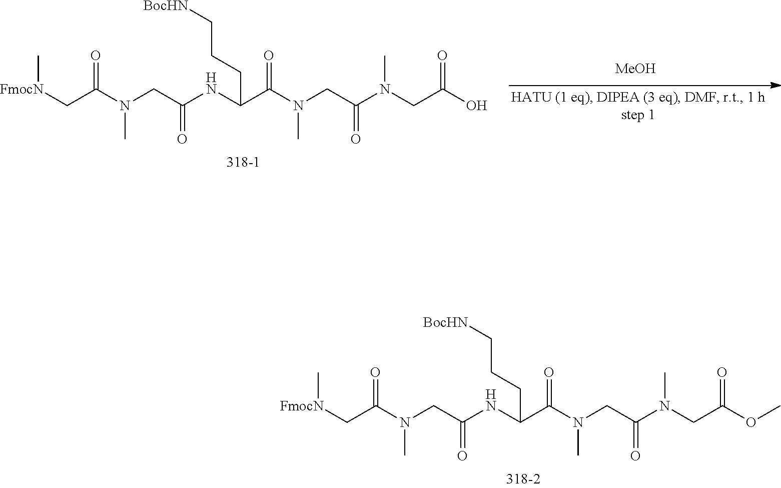

A solution of 318-1 (200 mg, 0.271 mmol), MeOH (43 mg, 1.342 mmol) and HATU (103 mg, 0.271 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 318-2 (167 mg, 0.222 mmol, 81.92%) as a white solid.

### Step 2

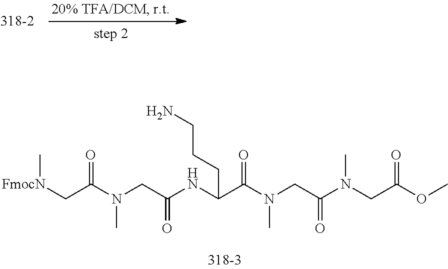

A solution of 318-2 (167 mg, 0.222 mmol) and TFA (1 mL) in anhydrous DCM (4 mL) was stirred at room temperature for 1 hr until LCMS indicated all starting amine was disappeared and desired product was detected. Then the solution was concentrated to dryness to 318-3 (135 mg, 0.207 mmol, 93.24%) as yellow oil, used as such in the next step.

### Step 3

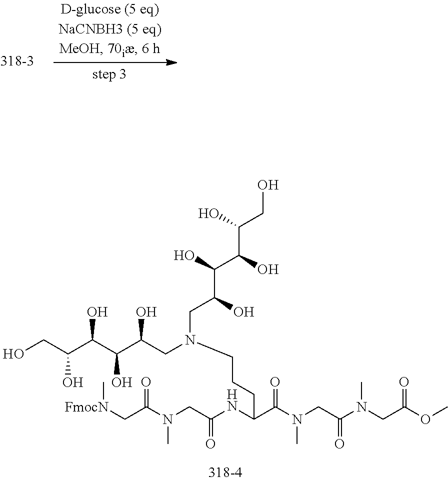

A solution of 318-3 (135 mg, 0.207 mmol) and D-Glucose (186 mg, 1.032 mmol) in anhydrous Methanol (50 mL) was heated at 50° C. for 30 min, then NaCNBH₃ (65 mg, 1.034 mmol) was added. The resulting solution was stirred for another 6 hr at 70° C. until indicated all starting amine was disappeared and the mass of desired product was detected. Then the reaction solution was concentrated and purified by reverse phase liquid chromatography to give 318-4 (121 mg, 0.123 mmol, 59.42%) as a white solid.

### Step 4

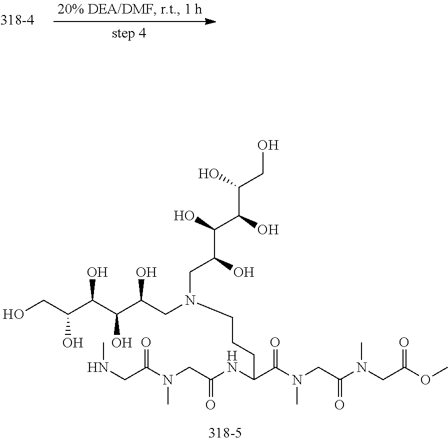 [SMILES: CNCC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC]

A solution of 318-4 (121 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 318-5 (75 mg, 0.099 mmol, 80.49%) as colorless oil, used as such in the next step.

### Step 5

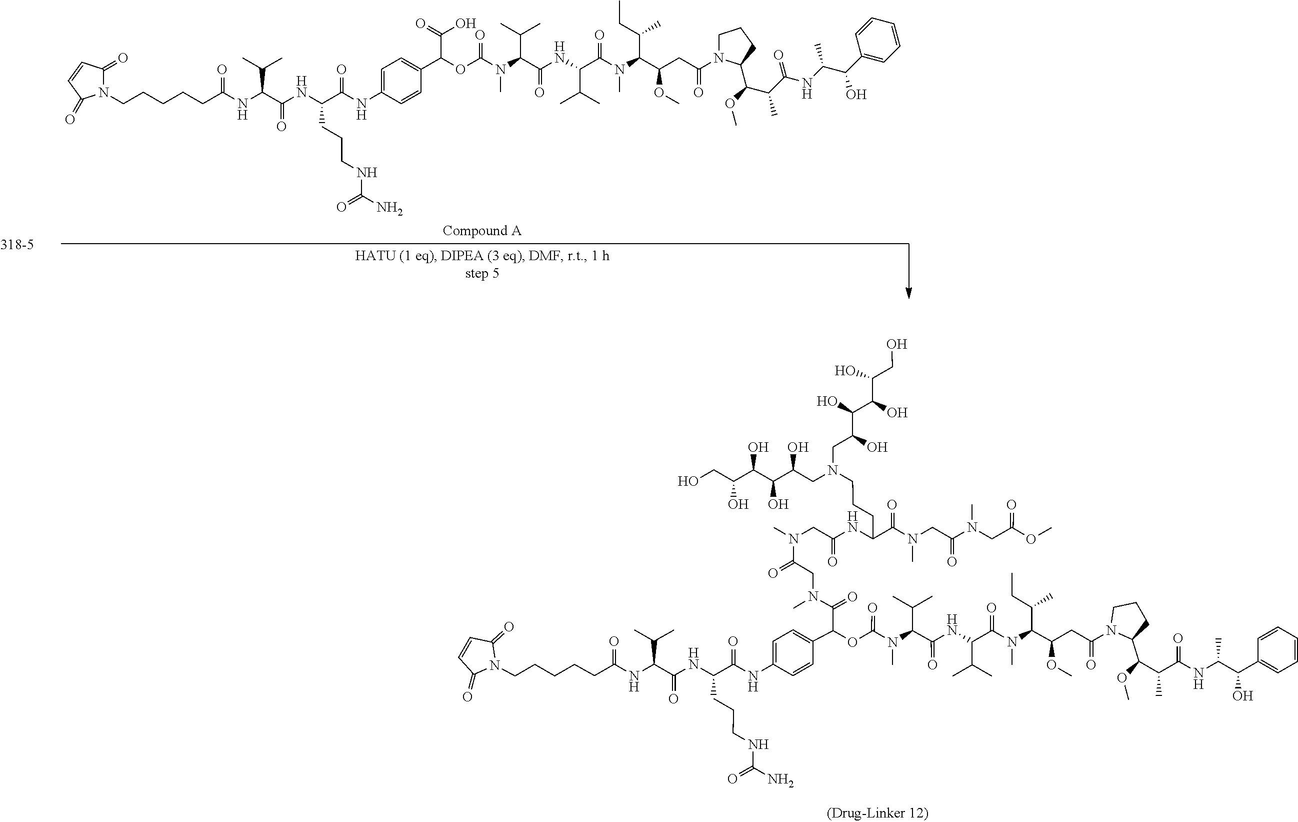

A solution of 318-5 (17 mg, 0.022 mmol), Compound A (30 mg, 0.022 mmol) and HATU (8 mg, 0.022 mmol) in anhydrous DMF (4 mL) was stirred at room temperature for 5 min, then DIPEA (8 mg, 0.062 mmol) was added. The resulting solution was stirred for another 1 hr at r.t. until LCMS indicated complete reaction. Then the reaction solution was purified by prep. HPLC to give drug-linker 12 (16 mg, 0.008 mmol, 36.36%) as a white solid. LCMS, m/z=1051.28 (M/2+H)⁺.

### Example 13: Preparation of Drug-Linker 13

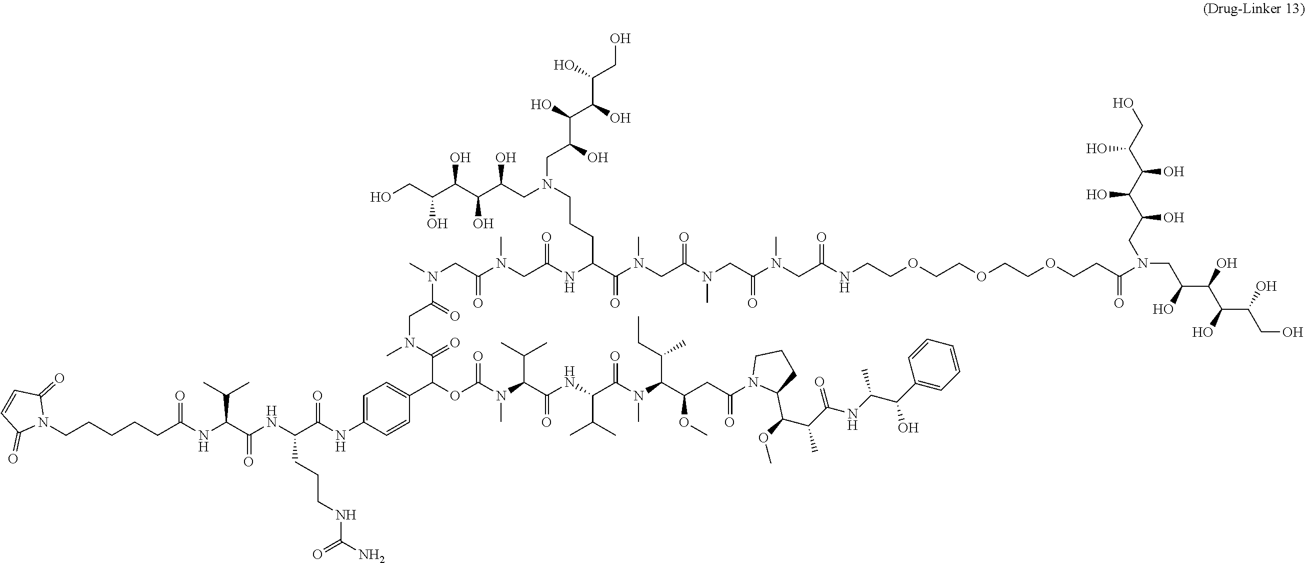 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

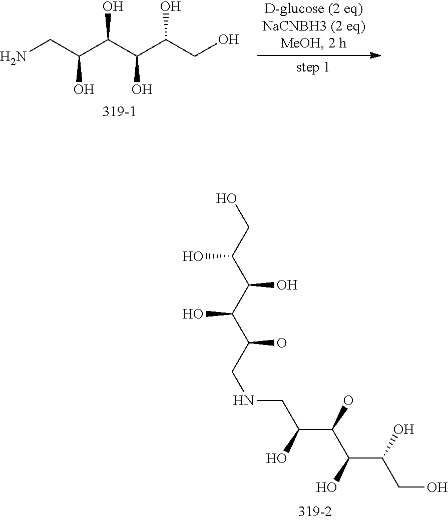

A solution of 319-1 (2.0 g, 0.011 mol) and D-Glucose (4.0 g, 0.022 mol) in anhydrous Methanol (50 mL) was heated at 50° C. for 30 min, then NaCNBH₃ (1.4 g, 0.022 mol) was added. The resulting solution was stirred for another 2 hr at 70° C. until indicated all starting amine was disappeared and the mass of desired product was detected. Then the reaction solution was concentrated and purified by reverse phase liquid chromatography to give 319-2 (2.6 g, 7.529 mmol, 68.44%) as a white solid.

### Step 2

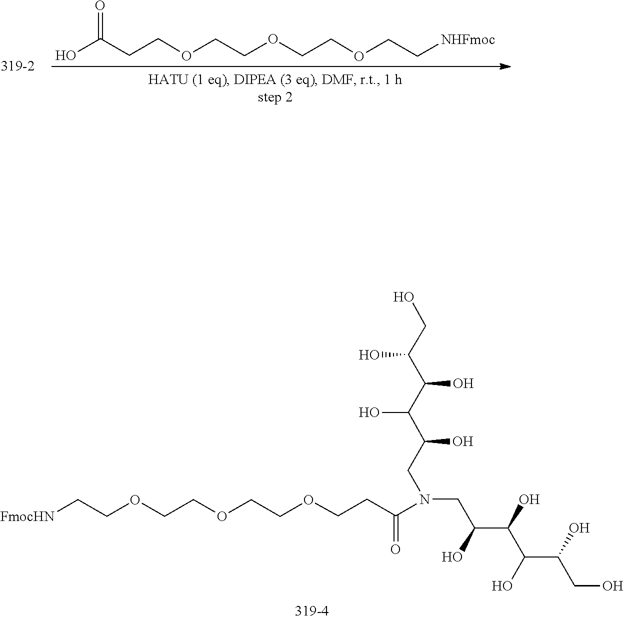

A solution of 319-2 (2.6 g, 7.529 mmol), 319-3 (3.3 g, 7.529 mmol) and HATU (2.9 g, 7.529 mmol) in anhydrous DMF (10 mL) was stirred at room temperature for 5 min, then DIPEA (2.9 g, 22.586 mmol) was added. The resulting solution was stirred for another 1 hr at r.t. until LCMS indicated complete reaction. The reaction solution was purified directly by reverse phase liquid chromatography to give 319-4 (1.5 g, 1.946 mmol, 25.85%) as a white solid.

### Step 3

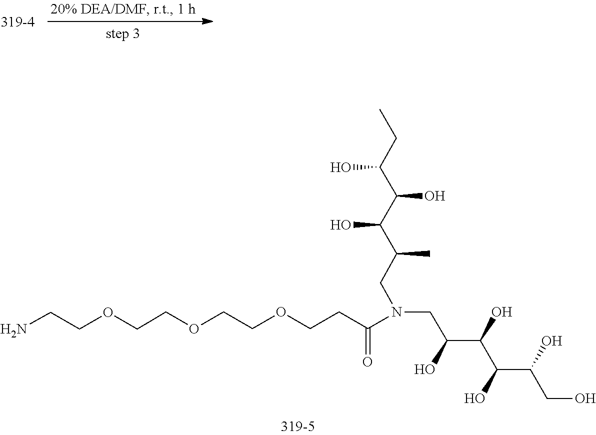 [SMILES: CC[C@@H](O)[C@@H](O)[C@H](O)[C@@H](C)CN(C[C@H](O)[C@@H](O)[C@H](O)[C@H](O)CO)C(=O)CCOCCOCCOCCN]

A solution of 319-4 (1.5 g, 1.946 mmol) and DEA (2 mL) in anhydrous DMF (8 mL) was stirred at room temperature for 1 hr until LCMS indicated all starting amine was disappeared and desired product was detected. Then the solution was concentrated to dryness to 319-5 (950 mg, 1.732 mmol, 89.00%) as colorless oil, used as such in the next step.

### Step 4

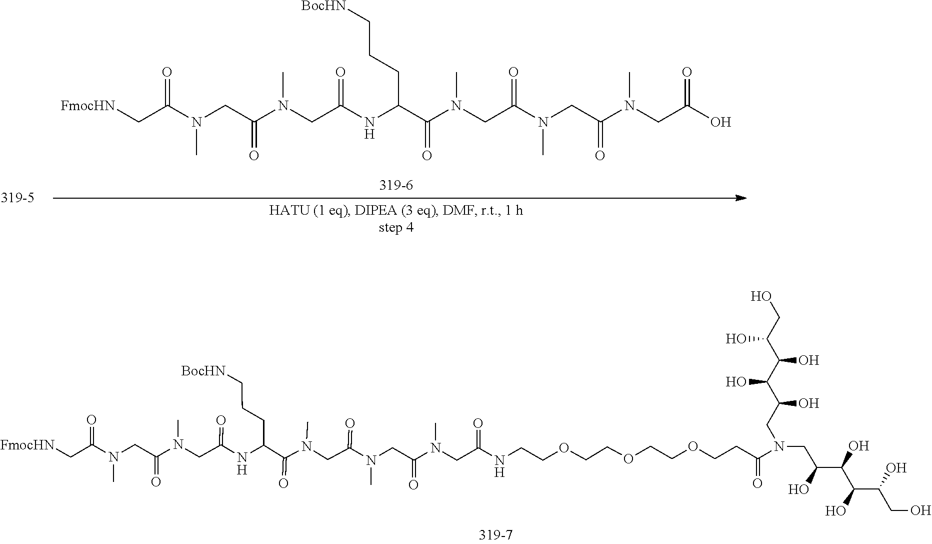

A solution of 319-5 (950 mg, 1.732 mmol), 319-6 (1501 mg, 1.732 mmol) and HATU (658 mg, 1.732 mmol) in anhydrous DMF (10 mL) was stirred at room temperature for 5 min, then DIPEA (670 mg, 5.184 mmol) was added. The resulting solution was stirred for another 1 hr at r.t. until LCMS indicated complete reaction. The reaction solution was purified directly by reverse phase liquid chromatography to give 319-7 (700 mg, 0.501 mmol, 28.93%) as a white solid.

### Step 5

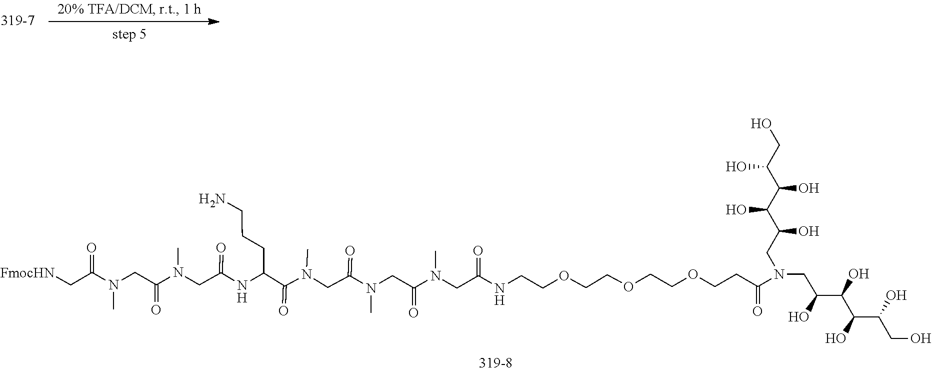

A solution of 319-7 (300 mg, 0.215 mmol) and TFA (1 mL) in anhydrous DCM (4 mL) was stirred at room temperature for 1 hr until LCMS indicated all starting amine was disappeared and desired product was detected. Then the solution was concentrated to dryness to 319-8 (245 mg, 0.189 mmol, 87.91%) as yellow oil, used as such in the next step.

### Step 6

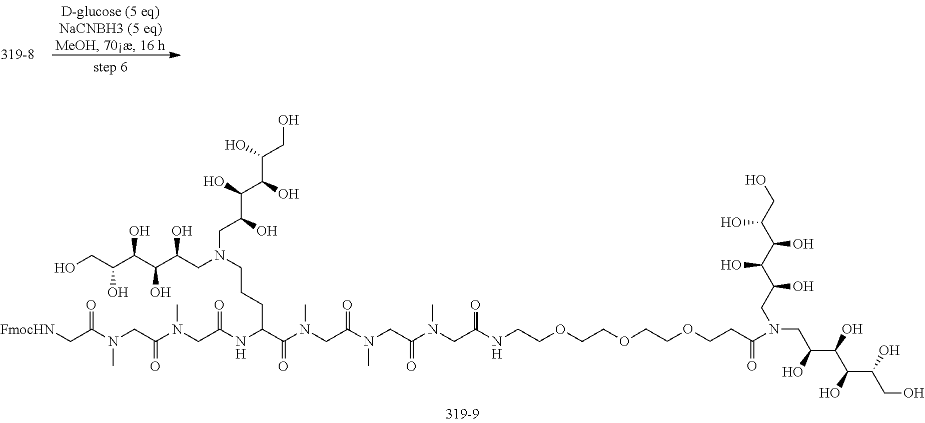

A solution of 319-8 (245 mg, 0.189 mmol) and D-Glucose (170 mg, 0.945 mmol) in anhydrous Methanol (50 mL) was heated at 50° C. for 30 min, then NaCNBH₃ (60 mg, 0.945 mmol) was added. The resulting solution was stirred for another 16 hr at 70° C. until indicated all starting amine was disappeared and the mass of desired product was detected. Then the reaction solution was concentrated and purified by reverse phase liquid chromatography to give 319-9 (140 mg, 0.086 mmol, 45.50%) as a white solid.

### Step 7

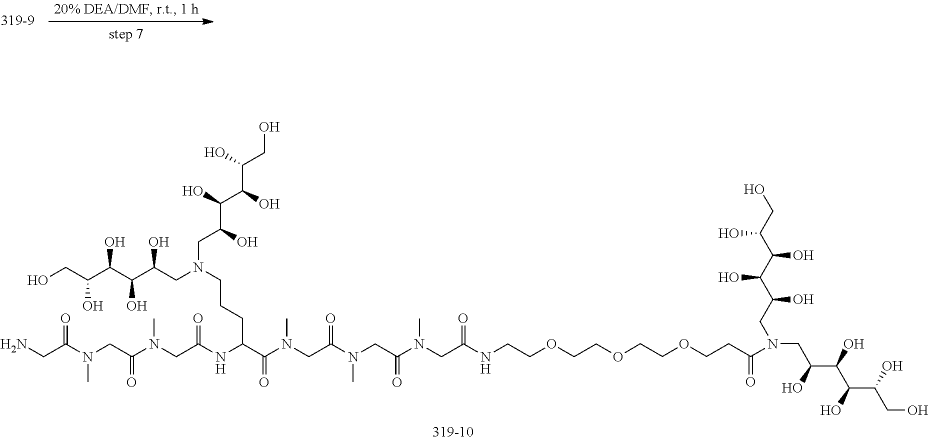 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 319-9 (140 mg, 0.086 mmol) and DEA (1 mL) in anhydrous DMF (4 mL) was stirred at room temperature for 1 hr until LCMS indicated all starting amine was disappeared and desired product was detected. Then the solution was concentrated to dryness to 319-10 (112 mg, 0.080 mmol, 93.02%) as colorless oil, used as such in the next step.

### Step 8

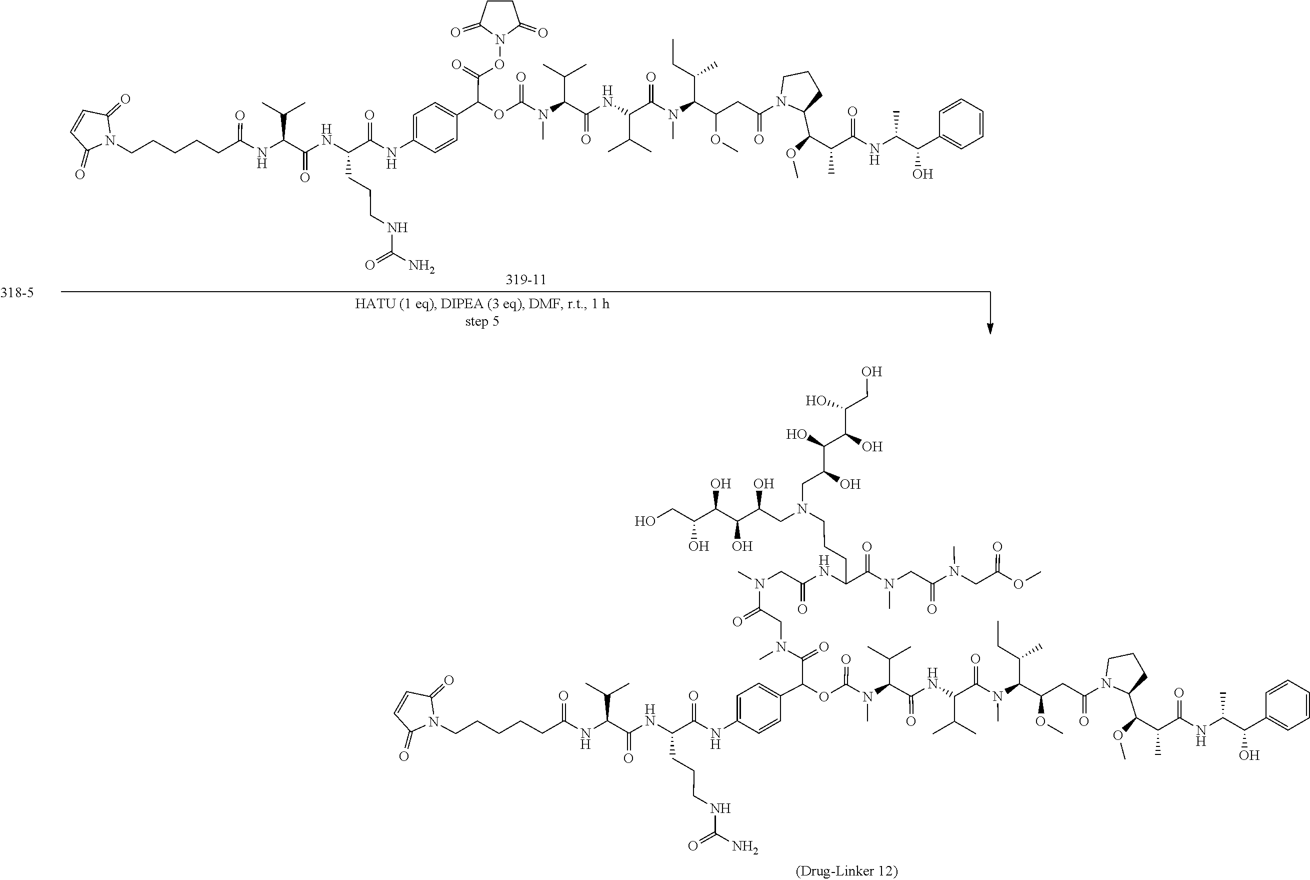

A solution of 319-10 (112 mg, 0.080 mmol) and 319-11 (116 mg, 0.080 mmol) in H₂O (3 mL) and MeCN (3 mL) was adjusted to pH=8 by aqueous sodium bicarbonate. The resulting solution was stirred for another 1 hr at r.t. until LCMS indicated complete reaction. Then the reaction solution was purified by prep. HPLC to give drug-linker 13 (8 mg, 0.003 mmol, 3.75%) as a white solid. LCMS, m/z=916.23 (M/3+H)⁺.

### Example 14: Preparation of Drug-Linker 14

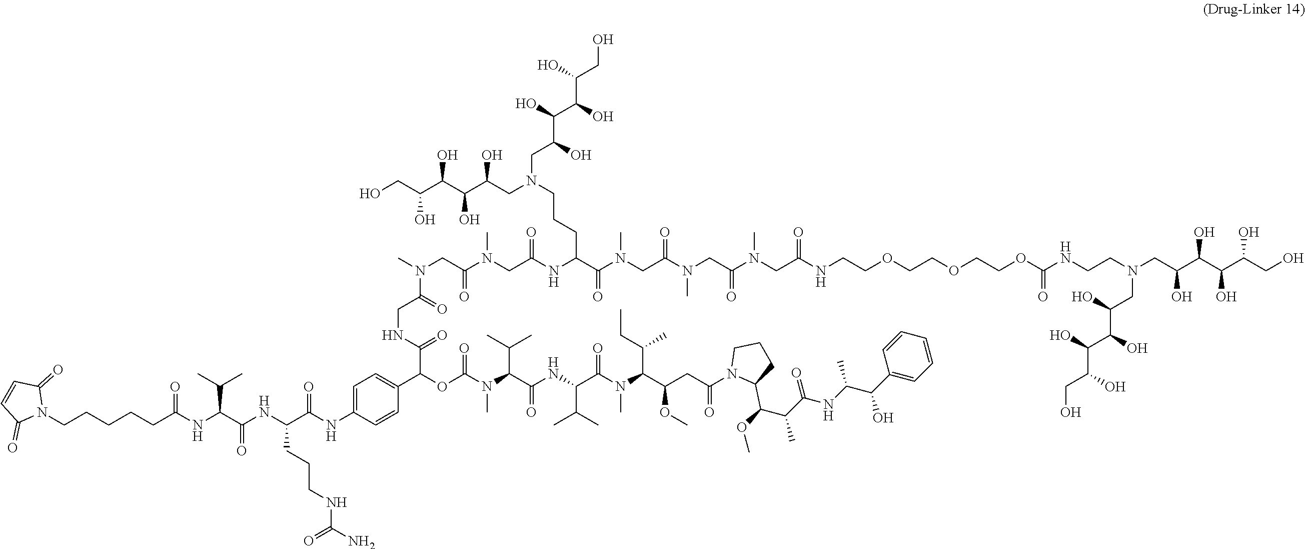 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

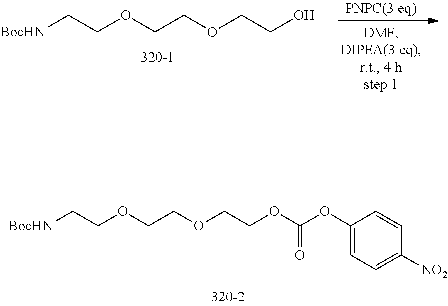 [SMILES: CC(C)(C)OC(=O)NCCOCCOCCO ; CC(C)(C)OC(=O)NCCOCCOCCOC(=O)Oc1ccc([N+](=O)[O-])cc1]

A solution of 320-1 (1.0 g, 4.011 mol) and DIPEA (1.6 g, 12.380 mol) in anhydrous DMF (15 mL) was stirred at room temperature for 5 min, then PNPC (3.7 g, 12.163 mmol) was added. The resulting solution was stirred for another 4 hr at r.t. until LCMS indicated all starting amine was disappeared and desired product was detected. Then the reaction solution was concentrated and purified by reverse phase liquid chromatography to give 320-2 (943 mg, 2.276 mmol, 56.73%) as a yellow solid.

### Step 2

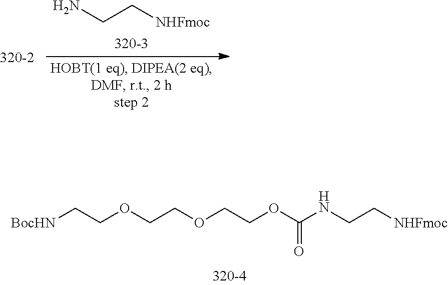

A solution of 320-2 (943 mg, 2.276 mmol), 320-3 (642 mg, 2.274 mmol) and HOBT (307 mg, 2.274 mmol) in anhydrous DMF (50 mL) was stirred at room temperature, then DIPEA (588 mg, 4.550 mmol) was added. The resulting solution was stirred for another 1 hr at r.t. until LCMS indicated all starting amine was disappeared and desired product was detected. The reaction solution was purified directly by reverse phase liquid chromatography to give 320-4 (850 mg, 1.524 mmol, 66.97%) as a white solid.

### Step 3

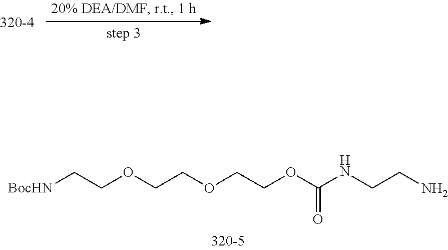 [SMILES: CC(C)(C)OC(=O)NCCOCCOCCOC(=O)NCCN]

A solution of 320-4 (850 mg, 1.524 mmol) and DEA (2 mL) in anhydrous DMF (8 mL) was stirred at room temperature for 1 hr until LCMS indicated all starting amine was disappeared and desired product was detected. Then the solution was concentrated to dryness to 320-5 (491 mg, 1.464 mmol, 96.06%) as colorless oil, used as such in the next step.

### Step 4

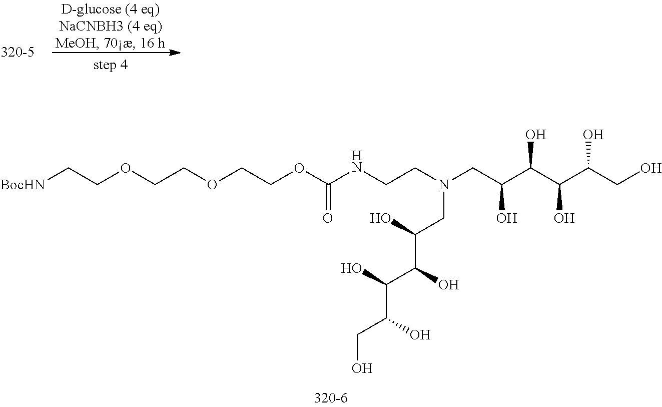 [SMILES: CC(C)(C)OC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-5 (491 mg, 1.464 mmol) and D-Glucose (1.05 g, 5.828 mmol) in anhydrous Methanol (50 mL) was heated at 50° C. for 30 min, then NaCNBH₃ (368 mg, 5.856 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-6 (470 mg, 0.708 mmol, 48.37%) as a white solid.

### Step 5

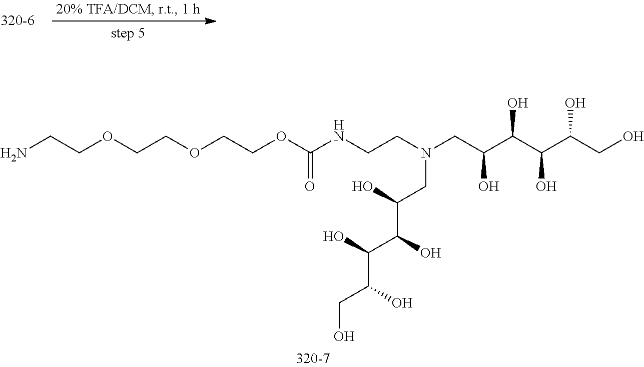 [SMILES: NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-6 (470 mg, 0.708 mmol) and TFA (2 mL) in anhydrous DCM (8 mL) was stirred at room temperature for 1 hr until LCMS indicated all starting amine was disappeared and desired product was detected. Then the solution was concentrated to dryness to 320-7 (385 mg, 0.683 mmol, 96.48%) as yellow oil, used as such in the next step.

### Step 6

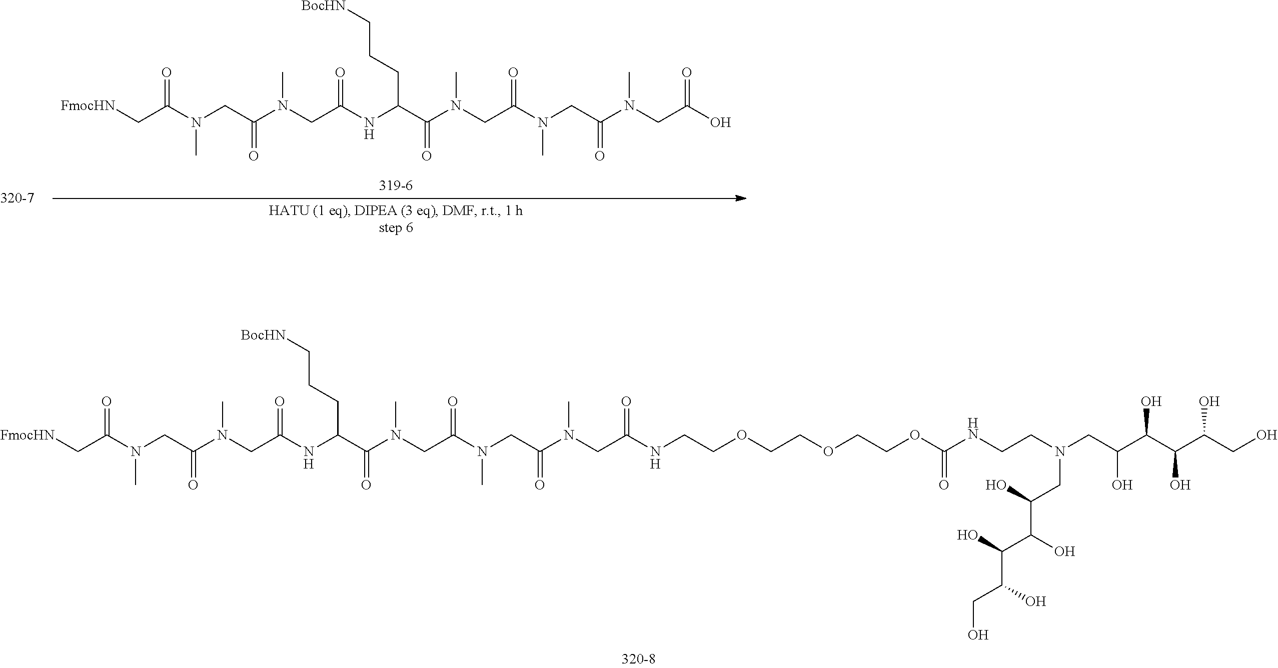

A solution of 320-7 (385 mg, 0.683 mmol), 319-6 (592 mg, 0.683 mmol) and HATU (260 mg, 0.684 mmol) in anhydrous DMF (6 mL) was stirred at room temperature for 5 min, then DIPEA (265 mg, 2.050 mmol) was added. The resulting solution was stirred for another 1 hr at r.t. until LCMS indicated complete reaction. The reaction solution was purified directly by reverse phase liquid chromatography to give 320-8 (413 mg, 0.292 mmol, 42.81%) as a white solid.

### Step 7

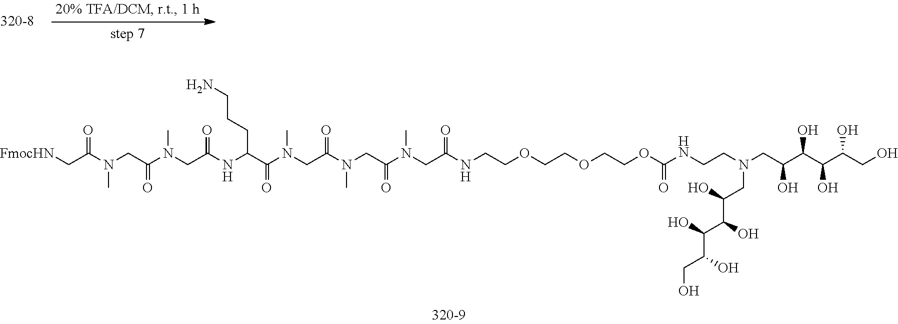

A solution of 320-8 (413 mg, 0.292 mmol) and TFA (2 mL) in anhydrous DCM (8 mL) was stirred at room temperature for 1 hr until LCMS indicated all starting amine was disappeared and desired product was detected. Then the solution was concentrated to dryness to 320-9 (370 mg, 0.262 mmol, 89.70%) as yellow oil, used as such in the next step.

### Step 8

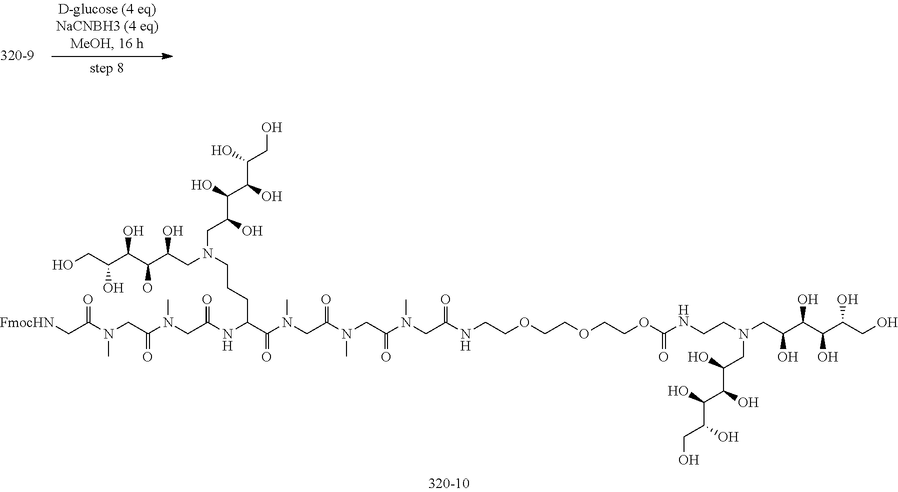

A solution of 320-9 (370 mg, 0.262 mmol) and D-Glucose (203 mg, 1.127 mmol) in anhydrous Methanol (40 mL) was heated at 50° C. for 30 min, then NaCNBH₃ (71 mg, 1.130 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-10 (210 mg, 0.128 mmol, 48.85%) as a white solid.

### Step 9

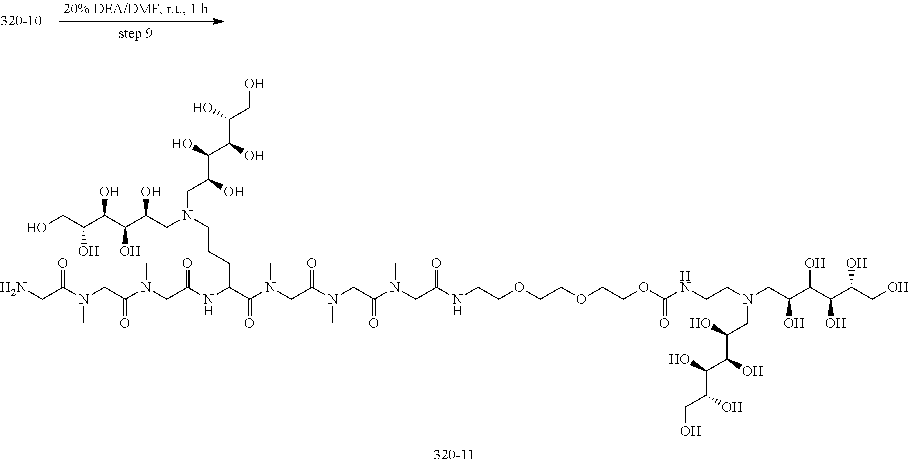 [SMILES: CN(CC(=O)NCCOCCOCCOC(=O)NCCN(C[C@H](O)C(O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 320-10 (210 mg, 0.128 mmol) and DEA (1 mL) in anhydrous DMF (4 mL) was stirred at room temperature for 1 hr until LCMS indicated all starting amine was disappeared and desired product was detected. Then the solution was concentrated to dryness to 320-11 (175 mg, 0.123 mmol, 96.38%) as colorless oil, used as such in the next step.

### Step 10

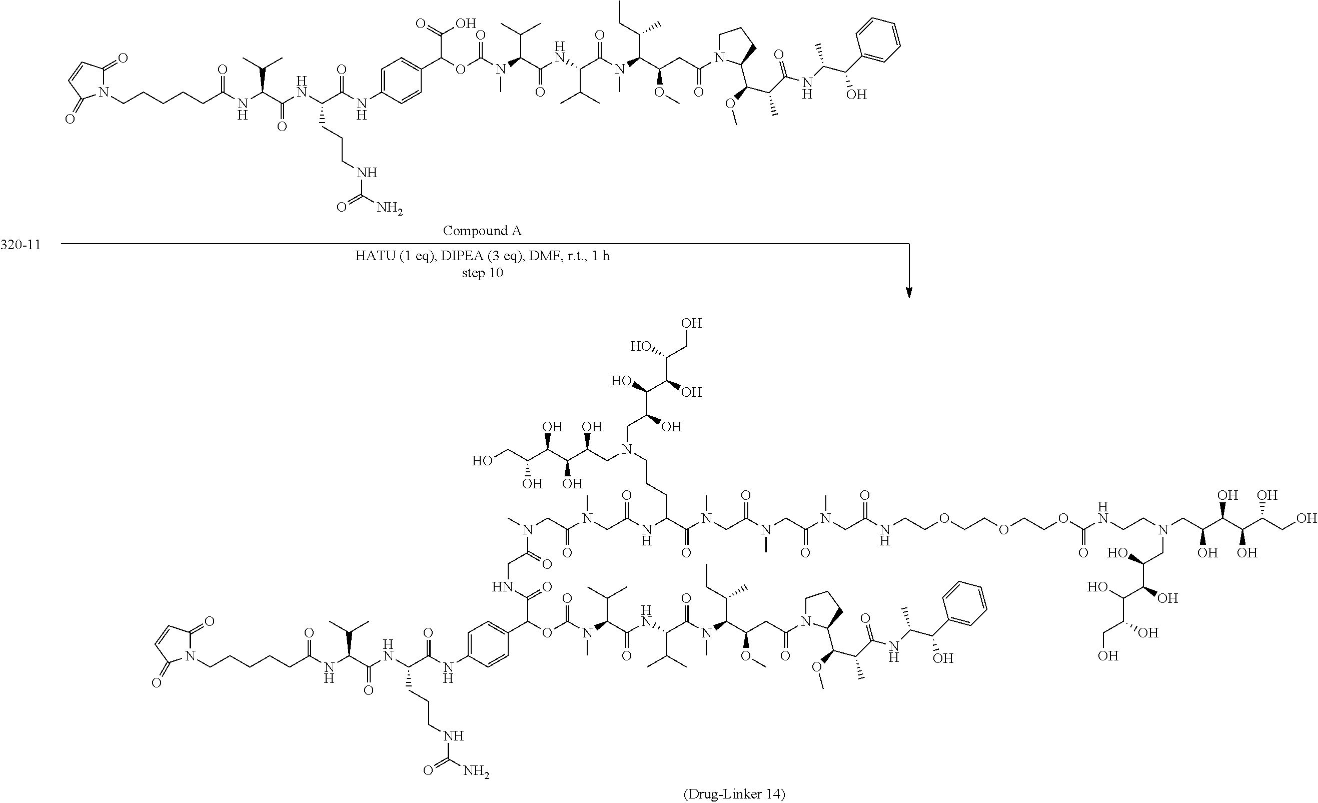

A solution of 320-11 (42 mg, 0.030 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 14 (16 mg, 0.006 mmol, 19.32%) as a white solid. LCMS, m/z=1381.18 (M/2+H)⁺.

### Example 15: Preparation of Drug-Linker 15

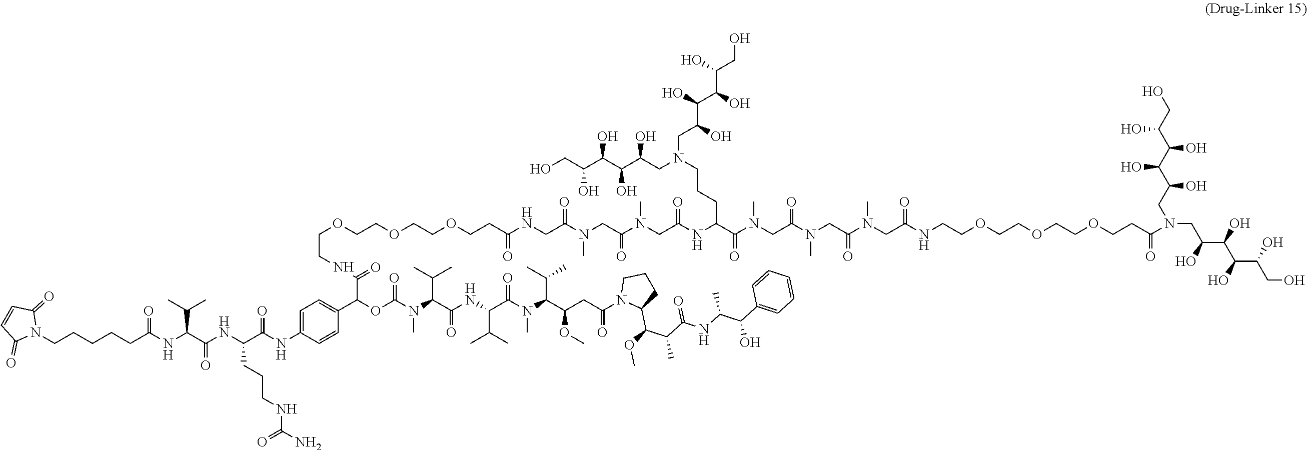

### Step 1

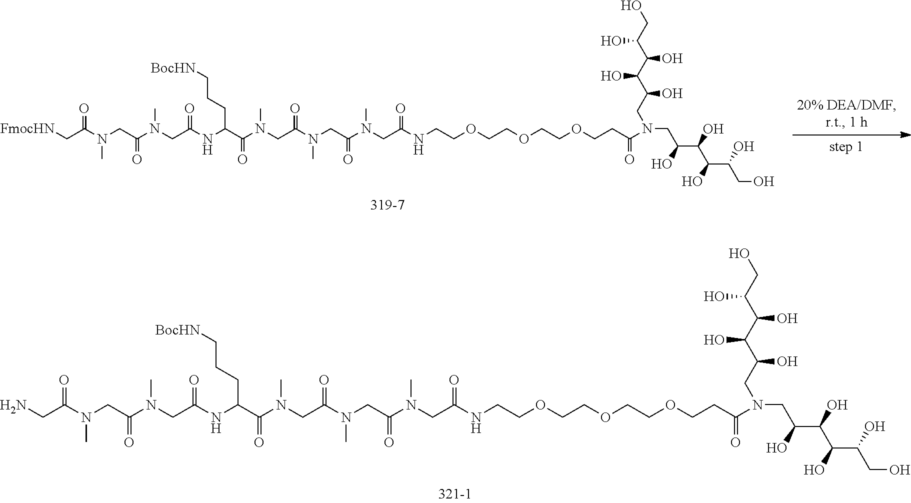

A solution of 319-7 (350 mg, 0.250 mmol) and DEA (1 mL) in anhydrous DMF (4 mL) was stirred at room temperature for 1 hour until LCMS indicated all starting amine was disappeared and desired product was detected. Then the solution was concentrated to dryness to 321-1 (280 mg, 0.238 mmol, 95.29%) as colorless oil, used as such in the next step.

### Step 2

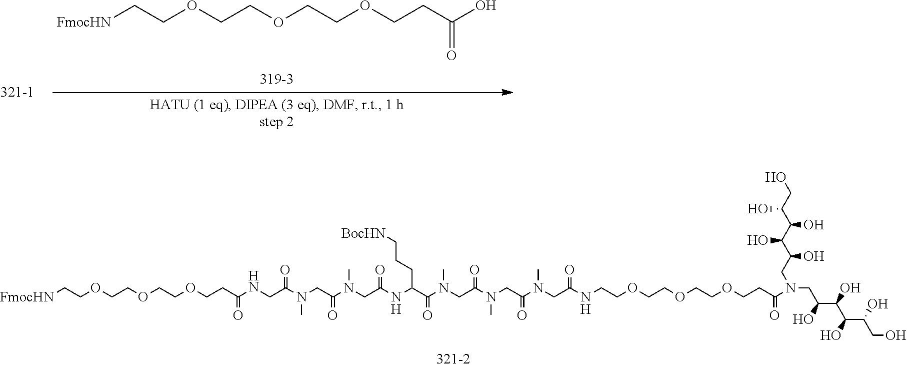

A solution of 321-1 (280 mg, 0.238 mmol), 319-3 (105 mg, 0.237 mmol) and HATU (90 mg, 0.237 mmol) in anhydrous DMF (10 mL) was stirred at room temperature for 5 min, then DIPEA (92 mg, 0.712 mmol) was added. The resulting solution was stirred for another 1 hour at r.t. until LCMS indicated complete reaction. The reaction solution was purified directly by reverse phase liquid chromatography to give 321-2 (265 mg, 0.166 mmol, 69.56%) as a white solid.

### Step 3

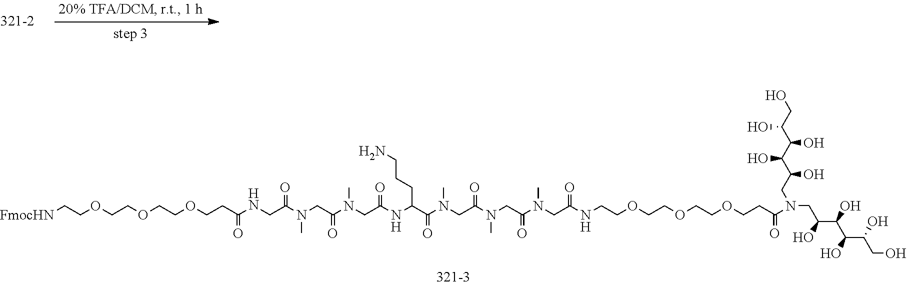

A solution of 321-2 (265 mg, 0.166 mmol) and TFA (1 mL) in anhydrous DCM (4 mL) was stirred at room temperature for 1 hr until LCMS indicated all starting amine was disappeared and desired product was detected. Then the solution was concentrated to dryness to 321-3 (243 mg, 0.162 mmol, 97.55%) as yellow oil, used as such in the next step.

### Step 4

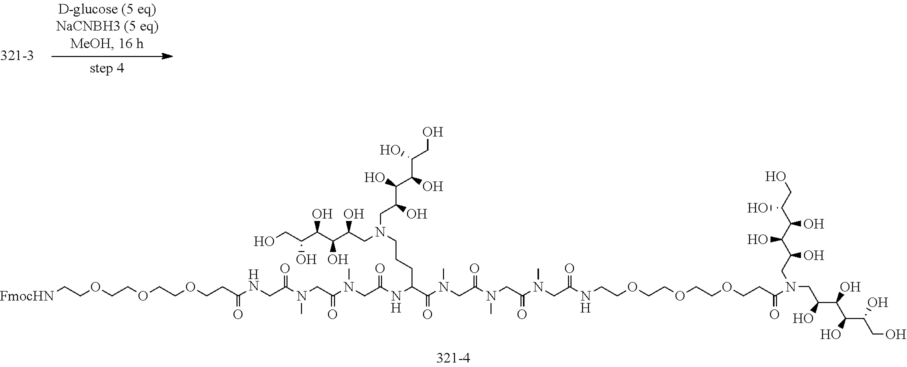

A solution of 321-3 (243 mg, 0.162 mmol) and D-Glucose (146 mg, 0.810 mmol) in anhydrous Methanol (50 mL) was heated at 50° C. for 30 min, then NaCNBH₃ (51 mg, 0.812 mmol) was added. The resulting solution was stirred for another 16 hr at 70° C. until indicated all starting amine was disappeared and the mass of desired product was detected. Then the reaction solution was concentrated and purified by reverse phase liquid chromatography to give 321-4 (155 mg, 0.085 mmol, 52.31%) as a white solid.

### Step 5

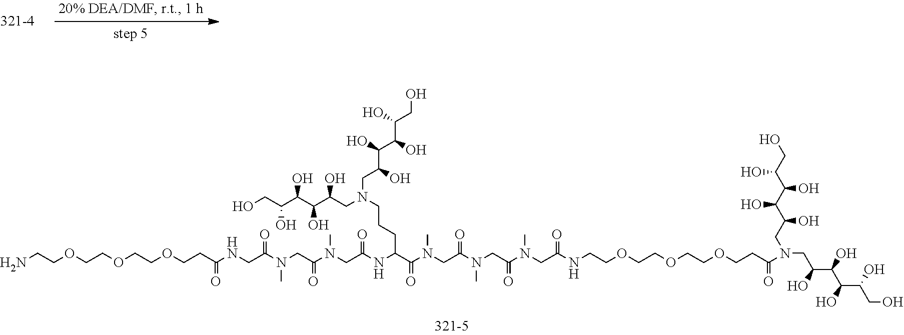 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CNC(=O)CCOCCOCCOCCN]

A solution of 321-4 (155 mg, 0.085 mmol) and DEA (1 mL) in anhydrous DMF (4 mL) was stirred at room temperature for 1 hr until LCMS indicated all starting amine was disappeared and desired product was detected. Then the solution was concentrated to dryness to 321-5 (130 mg, 0.081 mmol, 95.19%) as colorless oil, used as such in the next step.

### Step 6

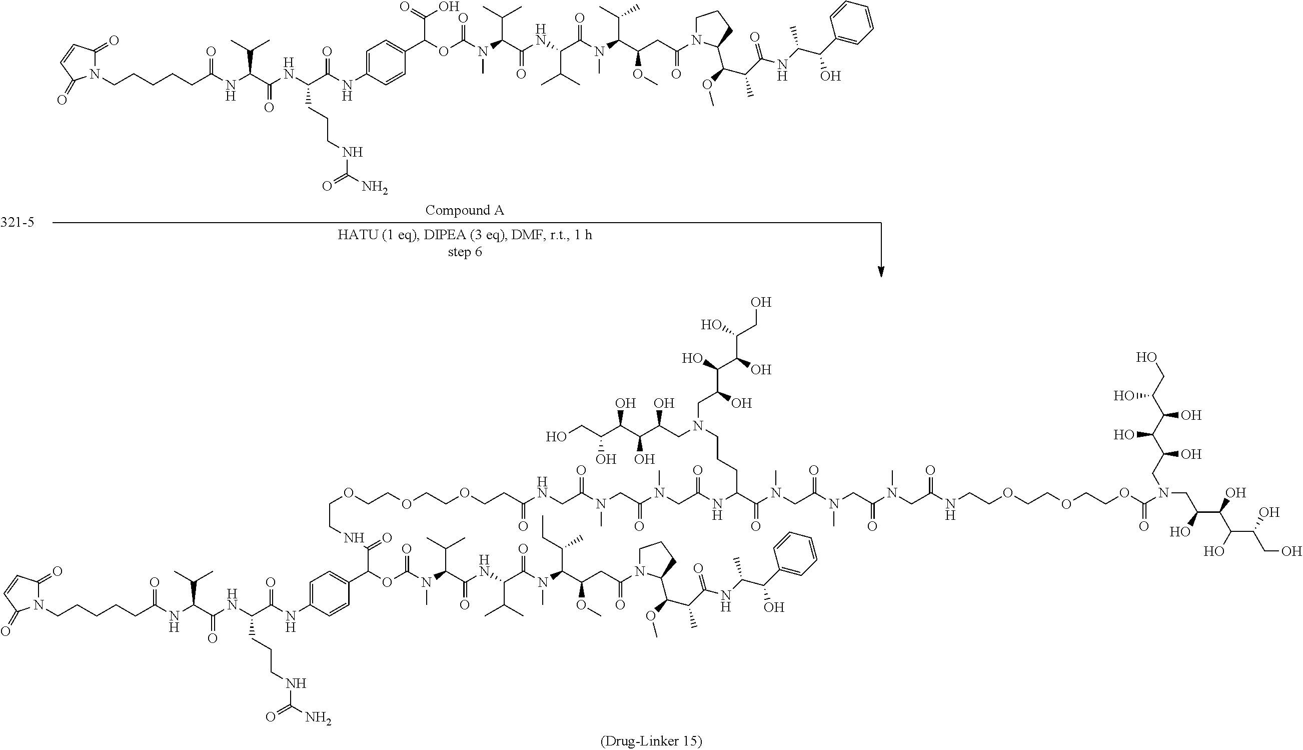

A solution of 321-5 (130 mg, 0.081 mmol), Compound A (110 mg, 0.081 mmol) and HATU (31 mg, 0.081 mmol) in anhydrous DMF (4 mL) was stirred at room temperature for 5 min, then DIPEA (31 mg, 0.240 mmol) was added. The resulting solution was stirred for another 1 hr at r.t. until LCMS indicated complete reaction. Then the reaction solution was purified by Prep. HPLC to give drug-linker 15 (24 mg, 0.008 mmol, 9.88%) as a white solid. LCMS, m/z=984.06 (M/3+H)⁺.

### Example 16: Preparation of Drug-Linker 16

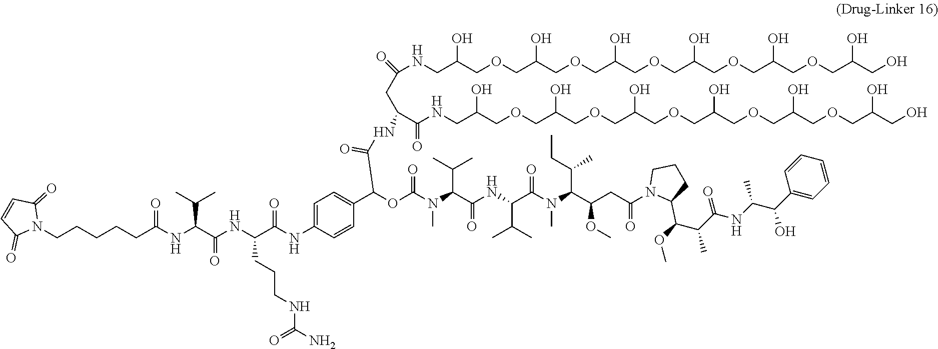 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

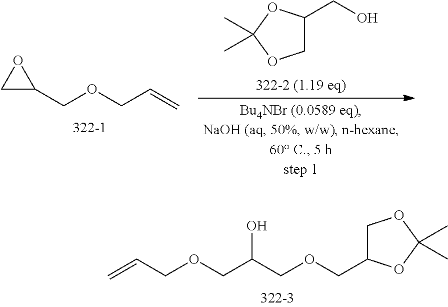

A solution of 322-2 (12.5 g, 94.64 mmol) and Bu₄NBr (1.51 g, 4.684 mmol) in mixture solvent of n-hexane (62.6 mL) and NaOH (aq, 50% w/w, 62.5 mL) was stirred at 60° C. A solution of 322-1 (9.07 g, 79.53 mmol) in n-hexane (12.5 mL) was dropped to the reaction mixture, and the resulting mixture was stirred at 60° C. for 5 hrs until most of 322-1 was consumed by TLC. The mixture was cooled down to r.t. and diluted with water (100 mL), then it was extracted with MTBE (200 mL×2). The organic parts were combined, dried over anhydrous Na₂SO₄, filtered and concentrated to dryness under reduced pressure. The crude product was purified with column chromatography (silica, 0-50% Ethyl acetate in petroleum ether) affording 322-3 as a colorless oil 20.68 g. ¹H NMR (400 MHz, CDCl₃) δ 5.93-5.80 (m, 1H), 5.30-5.20 (m, 1H), 5.19-5.12 (m, 1H), 4.30-4.15 (m, 1H), 4.05-3.91 (m, 4H), 3.73-3.66 (m, 1H), 3.58-3.43 (m, 6H), 2.74 (s, 1H), 1.39 (d, J=3.6 Hz, 3H), 1.33 (d, J=2.8 Hz, 3H).

### Step 2

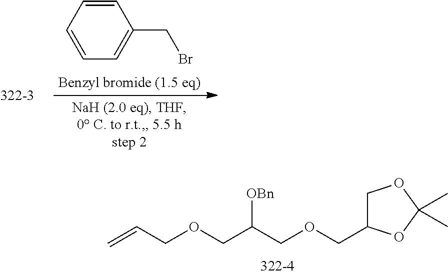 [SMILES: BrCc1ccccc1 ; C=CCOCC(COCC1COC(C)(C)O1)OCc1ccccc1]

To a mixture of NaH (5.58 g, 139.59 mmol) in anhydrous THE (340 mL) cooled in an ice bath was added a solution of 322-3 (17.18 g, 69.79 mmol) in anhydrous THE (85 mL) dropwise, then it was stirred at this temperature for 20 mins. Benzyl bromide (17.79 g, 104.69 mmol) was added dropwise, then the resulting mixture was allowed to warm to r.t. and stirred for 5 hrs until 322-3 was consumed by TLC. The reaction was quenched with saturated ammonium (150 mL) and extracted with ethyl acetate (200 mL×2). The organic phase was combined, dried over anhydrous Na₂SO₄, filtered and concentrated to dryness under reduced pressure to give the crude product, which was purified with column chromatography (silica, 0-40% ethyl acetate in petroleum ether) affording 322-4 (14.7 g, 43.73 mmol, 62.7%) as a colorless oil. Purity=90%-95%. 1H NMR (400 MHz, CDCl₃) δ 7.41-7.22 (m, 5H), 5.97-5.81 (m, 1H), 5.27 (dt, J=17.2, 1.8 Hz, 1H), 5.17 (dq, J=10.5, 1.5 Hz, 1H), 4.75-4.62 (m, 2H), 4.24 (t, J=6.0 Hz, 1H), 4.07-3.95 (m, 3H), 3.79-3.70 (m, 2H), 3.68-3.59 (m, 2H), 3.58-3.52 (m, 3H), 3.51-3.45 (m, 1H), 1.41 (s, 3H), 1.35 (s, 3H).

### Step 3

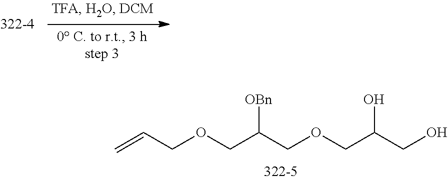 [SMILES: C=CCOCC(COCC(O)CO)OCc1ccccc1]

A solution of 322-4 (3.5 g, 10.411 mmol) in DCM (62.3 mL) and H₂O (0.78 mL) was stirred in an ice bath, then trifluoroacetic acid (1.56 mL) was added. The resulting solution was stirred for 3 hrs at this temperature until 322-4 was consumed by TLC. The reaction was quenched with saturated NaHCO₃ (aq) and then diluted with water (20 mL). The mixture was extracted with DCM (30 mL×2) and the organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-5 (2.815 g, 9.505 mmol, 91.3%) as a colorless oil. Purity=90%-95%. ¹H NMR (400 MHz, CDCl₃) δ 7.40-7.26 (m, 5H), 5.95-5.81 (m, 1H), 5.31-5.23 (m, 1H), 5.21-5.14 (m, 1H), 4.73-4.59 (m, 2H), 3.99 (dt, J=5.6, 1.5 Hz, 2H), 3.85-3.78 (m, 1H), 3.76-3.71 (m, 1H), 3.66-3.49 (m, 8H), 2.55 (s, 2H).

### Step 4

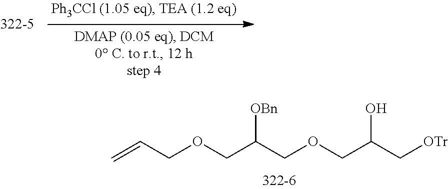

A solution of 322-5 (8.17 g, 27.586 mmol), TEA (3.349 g, 33.103 mmol) and DMAP (168.5 mg, 1.379 mmol) in DCM (40.8 mL) was stirred in an ice bath, then a solution of Ph₃CCl (8.075 g, 28.966 mmol) in DCM (40.8 mL) was added dropwise. The resulting mixture was gradually warmed to room temperature and stirred at ambient temperature for 12 hrs until most of 322-5 was consumed by TLC. The mixture was diluted with DCM (20 mL), washed with water (20 mL×2), dried over anhydrous Na₂SO₄, filtered and concentrated to dryness under reduced pressure to give the crude product, which was purified with column chromatography (silica, 0-40% Ethyl acetate in petroleum ether) affording 322-6 (13.86 g, 25.749 mmol, 93.3%) as a pale yellow oil. Purity=90%-95%. ¹H NMR (400 MHz, CDCl₃) δ 7.47-7.36 (m, 6H), 7.32-7.20 (m, 14H), 5.94-5.90 (m, 1H), 5.29-5.21 (m, 1H), 5.16 (dd, J=10.4, 1.6 Hz, 1H), 4.69-4.57 (m, 2H), 4.0-3.86 (m, 3H), 3.73-3.67 (m, 1H), 3.63-3.55 (m, 3H), 3.55-3.48 (m, 3H), 3.24-3.12 (m, 2H), 2.61 (s, 1H).

### Step 5

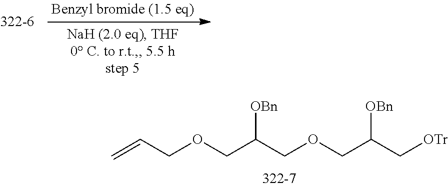

A mixture of NaH (2.06 g, 51.498 mmol) in anhydrous THE (90 mL) was stirred in an ice bath, then a solution of 322-6 (13.86 g, 25.749 mmol) in anhydrous THE (45 mL) was added dropwise. The reaction mixture was stirred at this temperature for 20 mins, then benzyl bromide (6.56 g, 38.62 mmol) was added. The resulting mixture was allowed to warm to r.t. and stirred for 5 hrs until 322-6 was consumed by TLC. The reaction was quenched with saturated ammonium (50 mL) and extracted with (ethyl acetate 60 mL×2). The organic phase was combined, dried over anhydrous Na₂SO₄, filtered and concentrated to dryness under reduced pressure to give the crude product, which was purified with column chromatography (silica, 0-30% ethyl acetate in petroleum ether) affording 322-7 (15.67 g, 24.940 mmol, 96.86%) as a colorless oil. Purity=90%-95%. ¹H NMR (400 MHz, CDCl₃) δ 7.51-7.40 (m, 6H), 7.35-7.20 (m, 19H), 5.94-5.80 (m, 1H), 5.29-5.21 (m, 1H), 5.17-5.10 (m, 1H), 4.65 (d, J=12.1 Hz, 4H), 3.99-3.87 (m, 2H), 3.79-3.65 (m, 2H), 3.65-3.59 (m, 2H), 3.58-3.53 (m, 2H), 3.52-3.46 (m, 2H), 3.23 (d, J=5.0 Hz, 2H).

### Step 6

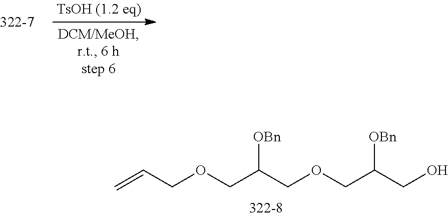 [SMILES: C=CCOCC(COCC(CO)OCc1ccccc1)OCc1ccccc1]

To a solution of 322-7 (15.67 g, 24.939 mmol) in a mixture solvent of DCM (62 mL) and MeOH (31 mL) was added TsOH·H₂O (5.7 g, 29.927 mmol), the resulting mixture was stirred for 6 hrs at room temperature until 322-7 was consumed by TLC. The reaction was quenched with saturated NaHCO₃ (aq.), diluted with water (30 mL), and then extracted with DCM (50 mL×3). The organic phase was combined, dried over anhydrous Na₂SO₄, filtered and concentrated to dryness under reduced pressure to give the crude product, which was purified with column chromatography (silica, 0-50% ethyl acetate in petroleum ether) affording 322-8 (9.09 g, 23.54 mmol, 94.37%) as a pale yellow oil. Purity=90%-95%. ¹H NMR (400 MHz, CDCl₃) δ 7.48-7.15 (m, 10H), 5.98-5.80 (m, 1H), 5.26 (d, J=17.2 Hz, 1H), 5.17 (d, J=10.4 Hz, 1H), 4.75-4.53 (m, 4H), 3.99 (d, J=5.6 Hz, 2H), 3.78-3.70 (m, 2H), 3.68-3.48 (m, 8H).

### Step 7

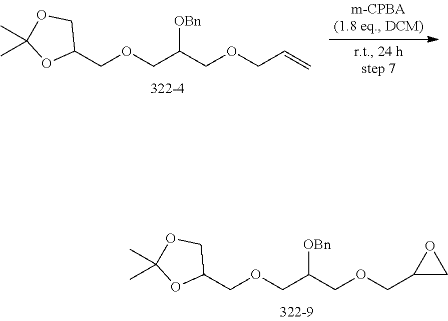

To a solution of 322-4 (14.0 g, 41.555 mmol) in DCM (36 mL) was added a solution of m-CPBA (12.91 g, 74.799 mmol) in DCM (103 mL) dropwise at room temperature. The resulting mixture was stirred at this temperature for 24 hours until 322-4 was consumed, and the reaction was quenched with saturated Na₂S₂O₃ (aq., 50 mL) and NaHCO₃ (aq, 50 mL). The reaction mixture was stirred for 30 mins, and then diluted with DCM (150 mL). The organic phase was washed with a mixture solution (60 mL×3) of saturated Na₂S₂O₃ and NaHCO₃ (aq, 1:1, VN), dried over anhydrous Na₂SO₄, filtered and concentrated to dryness under reduced pressure to give the crude product, which was purified with column chromatography (silica, 0-60% Ethyl acetate in petroleum ether) affording 322-9 (13.79 g, 39.16 mmol, 94.37%) as a pale yellow oil. Purity=90%-95%. ¹H NMR (400 MHz, CDCl₃) δ 7.45-7.16 (m, 5H), 4.69 (s, 2H), 4.25 (t, J=5.6 Hz, 1H), 4.04 (t, J=7.2 Hz, 1H), 3.86-3.69 (m, 3H), 3.68-3.36 (m, 7H), 3.19-3.08 (m, 1H), 2.83-2.76 (m, 1H), 2.63-2.53 (m, 1H), 1.42 (s, 3H), 1.37 (s, 3H).

### Step 8

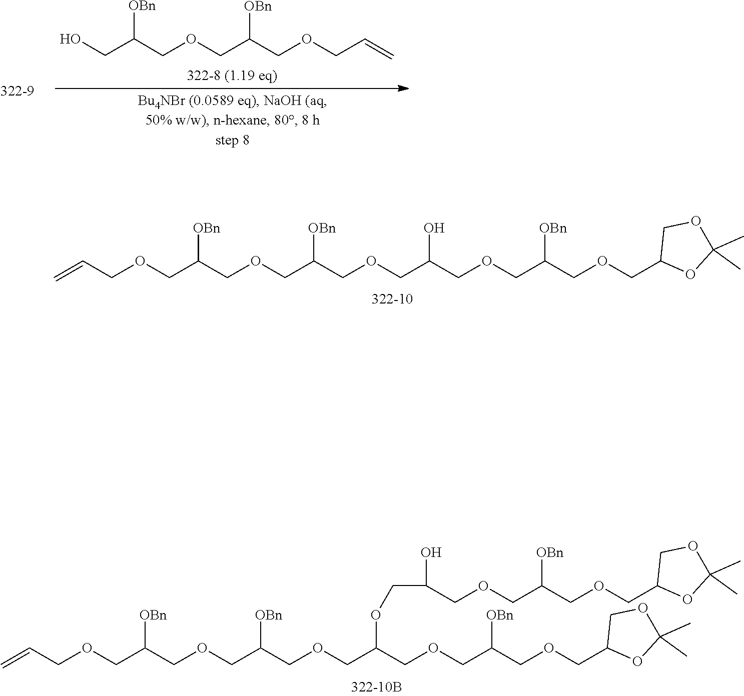

A solution of 322-8 (8.429 g, 23.934 mmol) and Bu₄NBr (454.4 mg, 1.409 mmol) in mixture solvent of n-hexane (30 mL) and NaOH (aq, 50% w/w, 30 mL) was stirred at 80° C. A mixture of 322-9 (11.0 g, 28.482 mmol) in n-hexane (6 mL) was dropped to the reaction mixture, and the resulting mixture was stirred at 80° C. for 8 hrs until most of 322-9 was consumed by TLC. The mixture was cooled down to r.t. and diluted with water (50 mL), then it was extracted with MTBE (100 mL×3). The organic parts were combined, dried over anhydrous Na₂SO₄, filtered and concentrated to dryness under reduced pressure. The crude product was purified with column chromatography (silica, 0-100% ethyl acetate in petroleum ether) affording 322-10 (8.0 g, 10.83 mmol, 45.3%) as a pale yellow oil and 322-10B (2.1 g, 1.925 mmol, 8.0%) as a pale yellow oil. ¹H NMR (400 MHz, CDCl₃) δ 7.38-7.15 (m, 15H), 5.96-5.73 (m, 1H), 5.31-5.06 (m, 2H), 4.76-4.51 (m, 6H), 4.29-4.13 (m, 1H), 4.07-3.86 (m, 4H), 3.76-3.37 (m, 22H), 2.52 (s, 1H), 1.56-1.05 (m, 6H). ¹H NMR (400 MHz, CDCl₃) δ 7.42-7.27 (m, 20H), 5.96-5.82 (m, 1H), 5.26 (dd, J=17.2, 1.8 Hz, 1H), 5.16 (dd, J=10.5, 1.9 Hz, 1H), 4.69-4.61 (m, 8H), 4.27-4.19 (m, 2H), 4.05-3.97 (m, 4H), 3.90-3.80 (m, 2H), 3.76-3.66 (m, 8H), 3.61-3.48 (m, 26H), 3.09 (s, 1H), 1.58-1.16 (m, 12H).

### Step 9

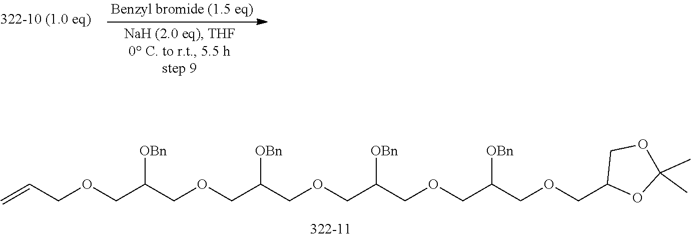 [SMILES: C=CCOCC(COCC(COCC(COCC(COCC1COC(C)(C)O1)OCc1ccccc1)OCc1ccccc1)OCc1ccccc1)OCc1ccccc1]

A mixture of NaH (867 mg, 21.668 mmol) in anhydrous THE (36 mL) was stirred in an ice bath, then a solution of 322-10 (8.0 g, 10.834 mmol) in anhydrous THE (20 mL) was added dropwise. The reaction mixture was stirred at this temperature for 20 mins, then benzyl bromide (2.76 g, 16.251 mmol) was added. The resulting mixture was allowed to warm to r.t. and stirred for 5 hrs until 322-10 was consumed by TLC. The reaction was quenched with saturated ammonium (25 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-11 (8.45 g, 10.20 mmol, 94.1%) as a pale yellow oil. Purity=90%-95%. ¹H NMR (400 MHz, CDCl₃) δ 7.43-7.25 (m, 20H), 5.99-5.82 (m, 1H), 5.27 (dd, J=17.2, 1.8 Hz, 1H), 5.17 (dd, J=10.4, 1.7 Hz, 1H), 4.77-4.60 (m, 8H), 4.30-4.18 (m, 1H), 4.06-3.96 (m, 3H), 3.81-3.70 (m, 5H), 3.63-3.47 (m, 18H), 1.47-1.21 (m, 6H).

### Step 10

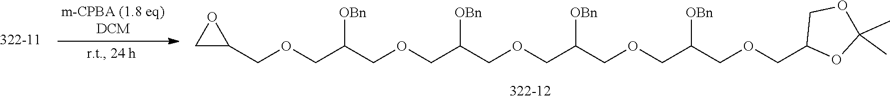 [SMILES: CC1(C)OCC(COCC(COCC(COCC(COCC(COCC2CO2)OCc2ccccc2)OCc2ccccc2)OCc2ccccc2)OCc2ccccc2)O1]

To a solution of 322-11 (8.45 g, 10.20 mmol) in DCM (12.8 mL) was added a solution of m-CPBA (3.16 g, 18.36 mmol) in DCM (25.6 mL) dropwise at room temperature. The resulting mixture was stirred at this temperature for 24 hours until 322-11 was consumed, and the reaction was quenched with saturated Na₂S₂O₃ (aq, 15 mL) and NaHCO₃ (aq, 15 mL). The reaction mixture was stirred for 30 mins, and then diluted with DCM (120 mL). The organic phase was washed with a mixture solution (40 mL×3) of saturated Na₂S₂O₃ and NaHCO₃ (aq, 1:1, VN), dried over anhydrous Na₂SO₄, filtered and concentrated to dryness under reduced pressure to give the crude product, which was purified with column chromatography (silica, 0-60% ethyl acetate in petroleum ether) affording 322-12 (6.858 g, 8.12 mmol, 79.6%) as a pale yellow oil. Purity=90%-95%. ¹H NMR (400 MHz, CDCl₃) δ 7.53-7.25 (m, 20H), 4.68 (s, 8H), 4.28-4.20 (m, 1H), 4.06-4.00 (m, 1H), 3.80-3.69 (m, 6H), 3.66-3.50 (m, 16H), 3.49-3.44 (m, 1H), 3.41-3.35 (m, 1H), 3.16-3.08 (m, 1H), 2.76 (t, J=4.6 Hz, 1H), 2.63-2.52 (m, 1H), 1.54-1.24 (m, 6H).

### Step 11

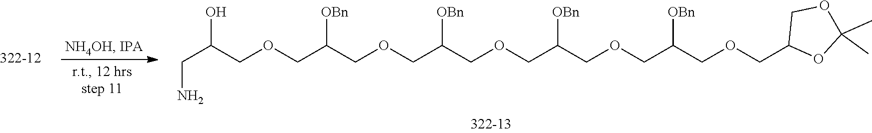 [SMILES: CC1(C)OCC(COCC(COCC(COCC(COCC(COCC(O)CN)OCc2ccccc2)OCc2ccccc2)OCc2ccccc2)OCc2ccccc2)O1]

To a solution of 322-12 (1.22 g, 1.445 mmol) in isopropyl alcohol (120 mL) was added ammonia (120 mL) dropwise at room temperature, and the resulting mixture was stirred at this temperature for 12 hours until 322-12 was consumed. The reaction mixture was concentrated to dryness under reduced pressure to afford 322-13 (1.28 g, 1.48 mmol, 100%) as a colorless oil, which was used in the next step without further purification. Purity=90%-95%.

### Step 12

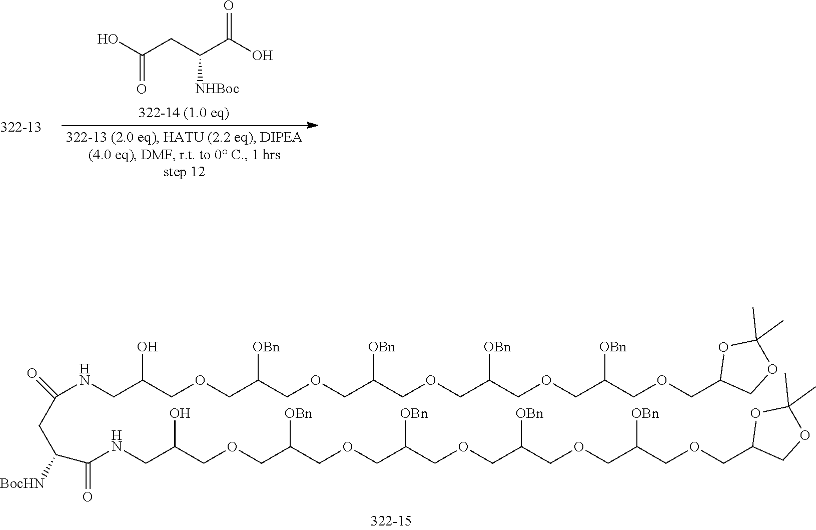 [SMILES: CC(C)(C)OC(=O)N[C@H](CC(=O)O)C(=O)O ; CC(C)(C)OC(=O)N[C@H](CC(=O)NCC(O)COCC(COCC(COCC(COCC(COCC1COC(C)(C)O1)OCc1ccccc1)OCc1ccccc1)OCc1ccccc1)OCc1ccccc1)C(=O)NCC(O)COCC(COCC(COCC(COCC(COCC1COC(C)(C)O1)OCc1ccccc1)OCc1ccccc1)OCc1ccccc1)OCc1ccccc1]

A solution of 322-14 (81.2 mg, 0.348 mmol) and HATU (291 mg, 0.766 mmol) in anhydrous DMF (5 mL) was stirred at room temperature for 15 mins, then it was stirred in an ice bath. A solution of 322-13 (600 mg, 0.696 mmol) in anhydrous DMF (3 mL) was added dropwise, followed by DIPEA (180 mg, 1.392 mmol). The resulting mixture was stirred in the ice bath for 1 h until most of 322-14 was consumed, and then diluted with ethyl acetate (60 mL). The organic phase was washed with H₂O (20 mL×3), dried over anhydrous Na₂SO₄, filtered and concentrated to dryness under reduced pressure to give the crude product, which was purified with column chromatography (silica, 0-100% ethyl acetate in petroleum ether) affording 322-15 (660 mg, 0.343 mmol, 98.7%) as a pale yellow oil. Purity=90%-95%. ¹H NMR (400 MHz, CDCl₃) δ 7.74-7.24 (m, 40H), 6.68-6.40 (m, 1H), 6.25-6.00 (m, 1H), 4.84-4.50 (m, 16H), 4.40 (s, 1H), 4.23 (t, J=6.0 Hz, 2H), 4.05-3.97 (m, 2H), 3.82-3.36 (m, 53H), 3.22-2.96 (m, 3H), 2.93-2.61 (m, 5H), 2.52-2.33 (m, 1H), 1.44-1.26 (m, 21H).

### Step 13

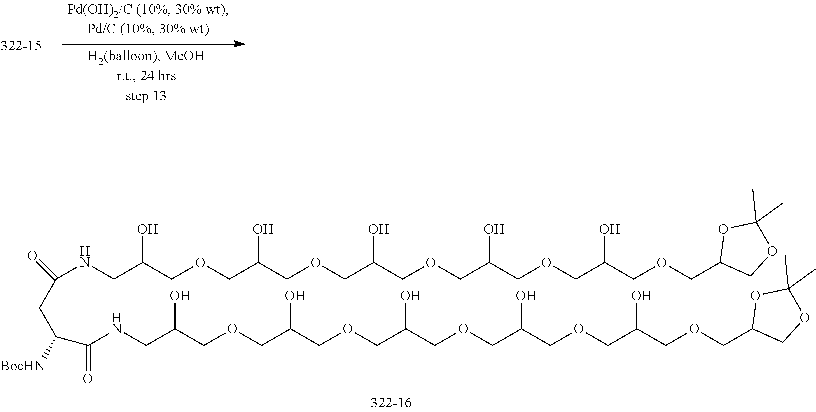 [SMILES: CC(C)(C)OC(=O)N[C@H](CC(=O)NCC(O)COCC(O)COCC(O)COCC(O)COCC(O)COCC1COC(C)(C)O1)C(=O)NCC(O)COCC(O)COCC(O)COCC(O)COCC(O)COCC1COC(C)(C)O1]

A mixture of 322-15 (160 mg, 0.0833 mmol), Pd(OH)₂/C (10%, 50 mg) and Pd/C (10%, 50 mg) in MeOH (10 mL) was stirred under hydrogen atmosphere (balloon) for 24 h at room temperature until 322-15 was completely converted into 322-16. The reaction mixture was filtered through a celite pad, and the filtrate was concentrated to dryness under reduced pressure to afford 322-16 (78.3 mg, 0.0652 mmol, 78.3%) as a colorless oil, which was used in the next step without further purification.

### Step 14

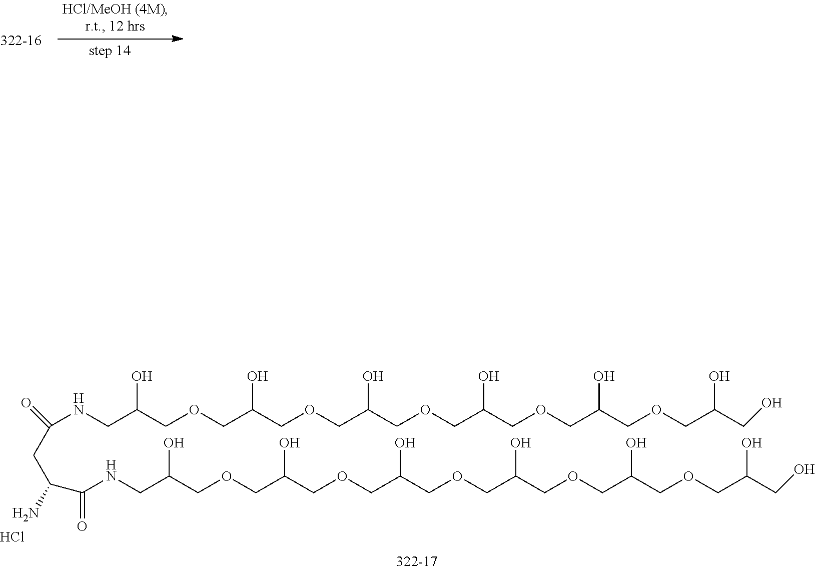 [SMILES: N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO ; Cl]

A mixture of 322-16 (78.3 mg, 0.0652 mmol) and HCl/MeOH (4M, 4 mL) was stirred at room temperature for 12 hours until 322-16 was completely converted into 322-17. The reaction mixture was concentrated to dryness under reduced pressure to afford 322-17 (65.0 mg, 0.0616 mmol, 94.4%) as an off-white solid, which was used in the next step without further purification.

### Step 15

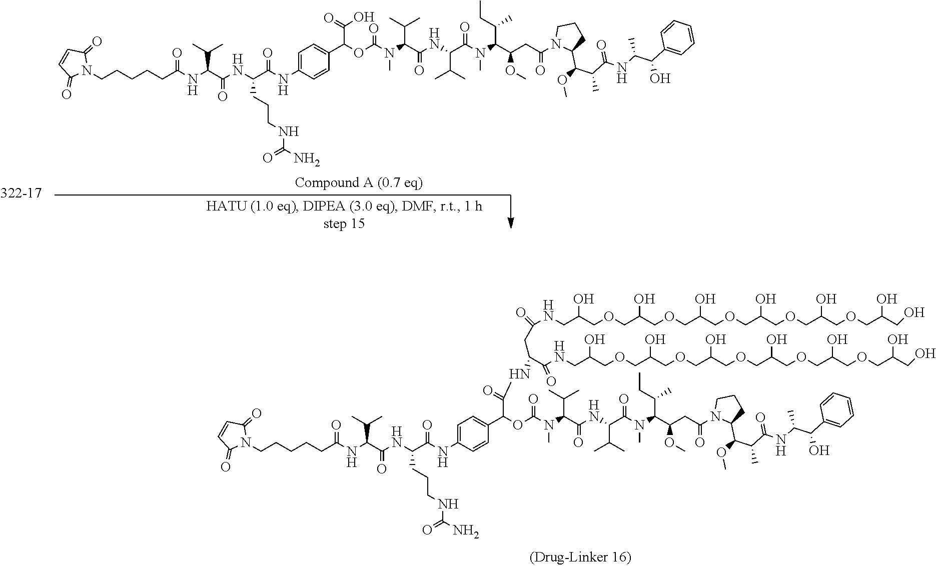

A solution of 322-17 (60 mg, 0.0568 mmol), Compound A (54 mg, 0.0397 mmol), HATU (21.6 mg, 0.0568 mmol) and DIPEA (22 mg, 0.170 mmol) in anhydrous DMF (4 mL) was stirred at room temperature for 1 h until Compound A was consumed.

Then the reaction solution was purified by prep-HPLC to give drug-linker 16 (18.1 mg, 0.00766 mmol, 19.3%) as a white solid. LCMS, m/z=1182.02 (M/2+H)⁺, m/z=788.25 (M/3+H)⁺.

### Example 17: Preparation of Drug-Linker 17

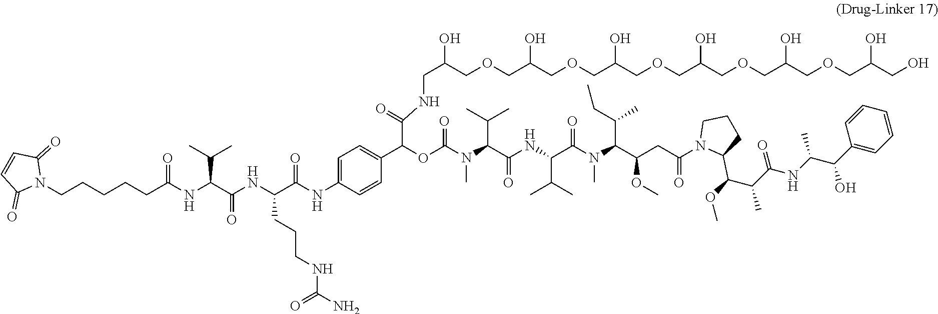 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

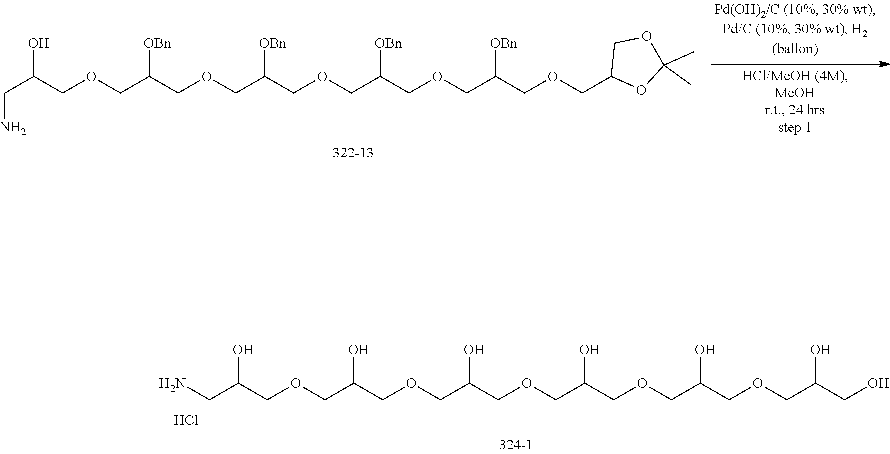 [SMILES: CC1(C)OCC(COCC(COCC(COCC(COCC(COCC(O)CN)OCc2ccccc2)OCc2ccccc2)OCc2ccccc2)OCc2ccccc2)O1 ; NCC(O)COCC(O)COCC(O)COCC(O)COCC(O)COCC(O)CO ; Cl]

A mixture of 322-13 (160 mg, 0.186 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (balloon) for 24 h at room temperature until 322-13 was completely converted into 324-1. The reaction mixture was filtered through a celite pad, and the filtrate was concentrated to dryness under reduced pressure to afford 324-1 (92.3 mg, 0.186 mmol, 100%) as an off-white solid, which was used in the next step without further purification.

### Step 2

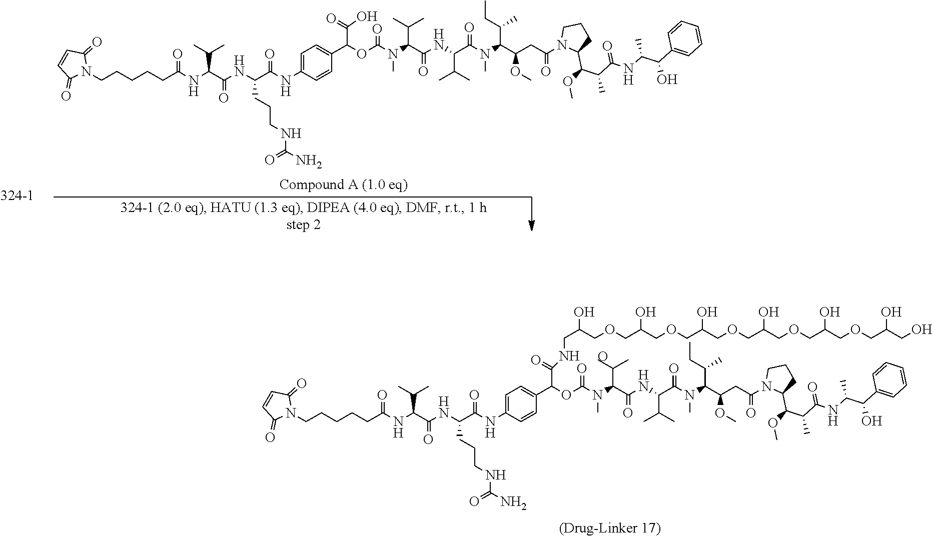

A solution of 324-1 (44 mg, 0.0885 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed.

Then the reaction solution was purified by prep-HPLC to give drug-linker 17 (28.0 mg, 0.0155 mmol, 35.2%) as a white solid. LCMS, m/z=1804.11 (M+H)⁺, m/z=902.76 (M/2+H)⁺.

### Example 18: Preparation of Drug-Linker 18

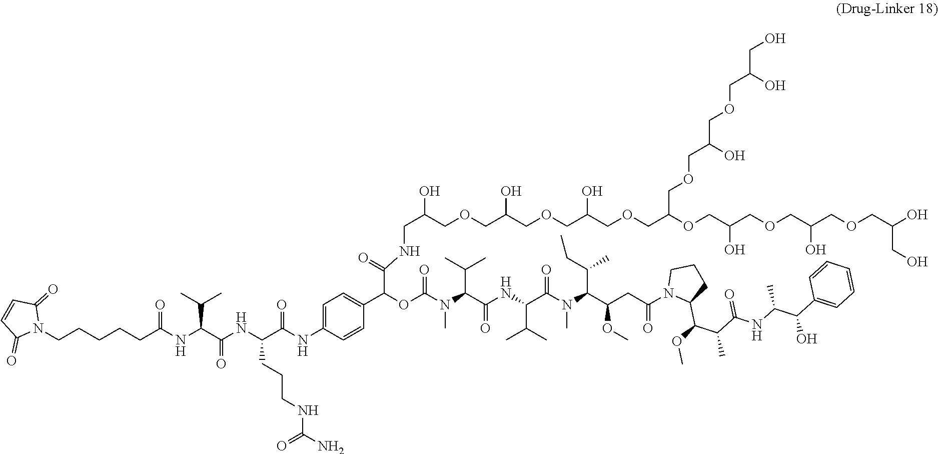 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(COCC(O)COCC(O)CO)OCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

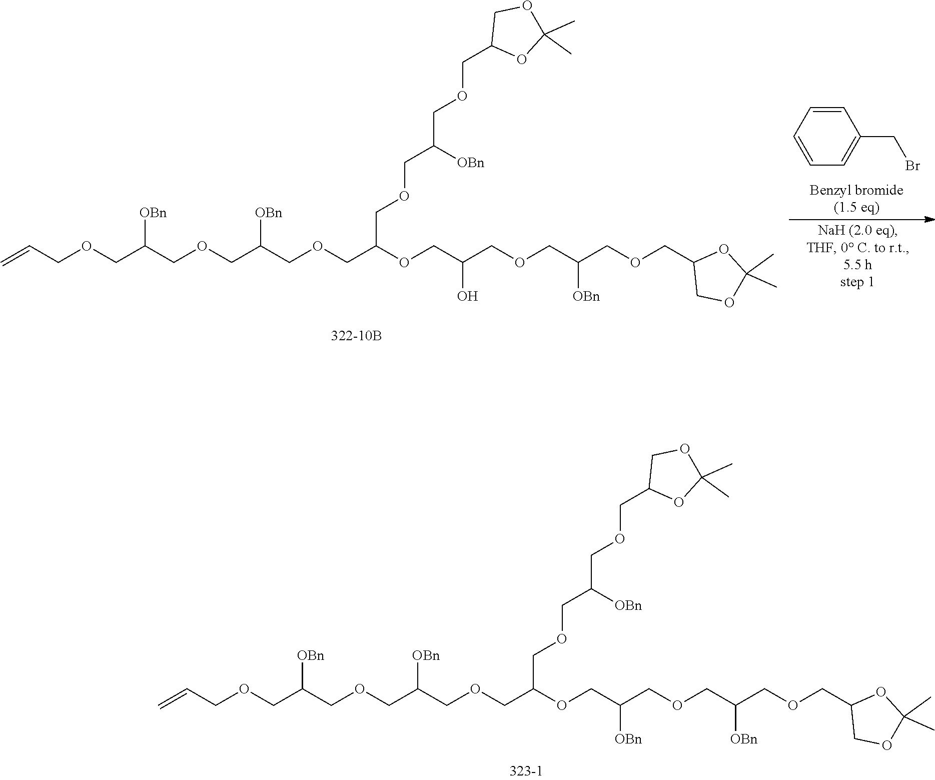

A mixture of NaH (205 mg, 5.13 mmol) in anhydrous THE (12.6 mL) was stirred in an ice bath, then a solution of 322-10B (2.79 g, 2.565 mmol) in anhydrous THE (7 mL) was added dropwise. The reaction mixture was stirred at this temperature for 20 mins, then benzyl bromide (654 g, 3.847 mmol) was added. The resulting mixture was allowed to warm to r.t. and stirred for 5 hrs until 322-10B was consumed by TLC. The reaction was quenched with saturated ammonium (15 mL) and extracted with ethyl acetate (30 mL×2). The organic phase was combined, dried over anhydrous Na₂SO₄, and concentrated to dryness under reduced pressure to give the crude product, which was purified with column chromatography (silica, 0-80% ethyl acetate in petroleum ether) affording 323-1 (2.772 g, 2.348 mmol, 91.8%) as a pale yellow oil. Purity=90%-95%.

### Step 2

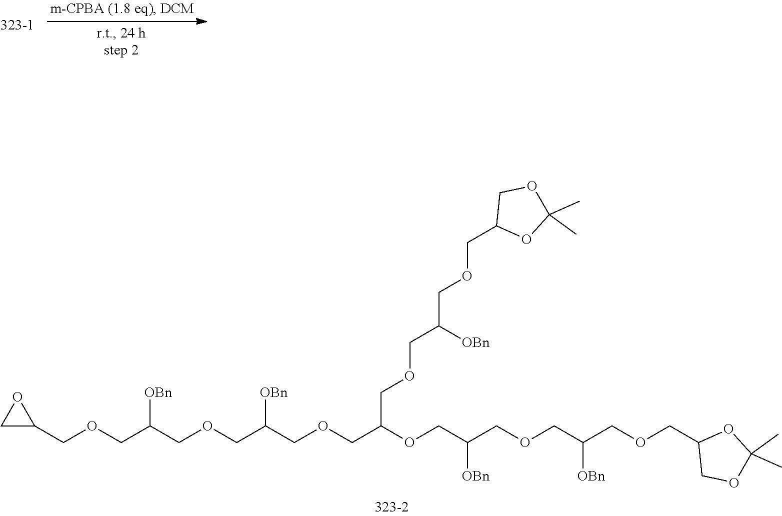 [SMILES: CC1(C)OCC(COCC(COCC(COCC(COCC(COCC2CO2)OCc2ccccc2)OCc2ccccc2)OCC(COCC(COCC2COC(C)(C)O2)OCc2ccccc2)OCc2ccccc2)OCc2ccccc2)O1]

To a solution of 323-1 (2.77 g, 2.346 mmol) in DCM (4.2 mL) was added a solution of m-CPBA (729 mg, 4.223 mmol) in DCM (8.4 mL) dropwise at room temperature. The resulting mixture was stirred at this temperature for 24 hours until 323-1 was consumed, and the reaction was quenched with saturated Na₂S₂O₃ (aq., 10 mL) and NaHCO₃(aq, 10 mL). The reaction mixture was stirred for 30 mins, and then diluted with DCM (100 mL). The organic phase was washed with a mixture solution (30 mL×3) of saturated Na₂S₂O₃ and NaHCO₃(aq, 1:1, VN), dried over anhydrous Na₂SO₄, filtered and concentrated to dryness under reduced pressure to give the crude product, which was purified with column chromatography (silica, 0-100% ethyl acetate in petroleum ether) affording 323-2 (2.147 g, 1.79 mmol, 76.5%) as a pale yellow oil. Purity=90%-95%. ¹H NMR (400 MHz, CDCl₃) δ 7.50-7.20 (m, 25H), 4.65 (s, 10H), 4.22 (t, J=6.0 Hz, 2H), 4.06-3.98 (m, 2H), 3.82-3.30 (m, 38H), 3.15-3.03 (m, 1H), 2.82-2.70 (m, 1H), 2.62-2.51 (m, 1H), 1.46-1.28 (m, 12H).

### Step 3

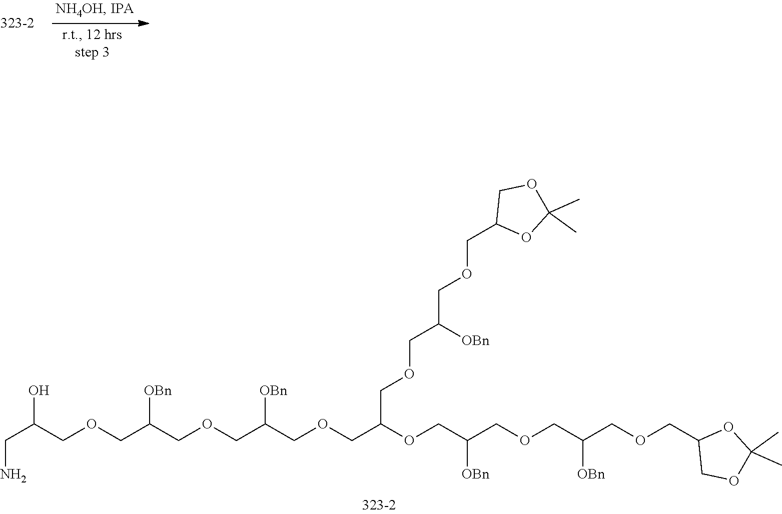 [SMILES: CC1(C)OCC(COCC(COCC(COCC(COCC(COCC(O)CN)OCc2ccccc2)OCc2ccccc2)OCC(COCC(COCC2COC(C)(C)O2)OCc2ccccc2)OCc2ccccc2)OCc2ccccc2)O1]

To a solution of 323-2 (450 mg, 0.376 mmol) in isopropyl alcohol (32 mL) was added ammonia (32 mL) dropwise at room temperature, and the resulting mixture was stirred at this temperature for 12 hours until 323-2 was consumed. The reaction mixture was concentrated to dryness under reduced pressure to afford 323-3 (456 mg, 1.48 mmol, 100%) as a colorless oil, which was used in the next step without further purification. Purity=90%-95%.

### Step 4

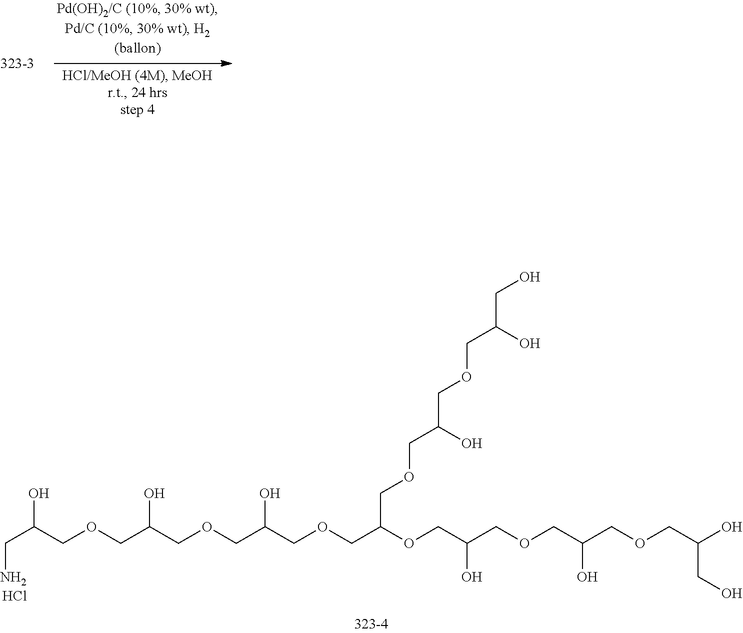 [SMILES: NCC(O)COCC(O)COCC(O)COCC(COCC(O)COCC(O)CO)OCC(O)COCC(O)COCC(O)CO ; Cl]

A mixture of 323-3 (250 mg, 0.206 mmol), Pd(OH)₂/C (10%, 75 mg) and Pd/C (10%, 75 mg) in a mixed solvent of MeOH (16 mL) and HCl/MeOH (4M, 4 mL) was stirred under hydrogen atmosphere (balloon) for 24 h at room temperature until 323-3 was completely converted into 323-4. The reaction mixture was filtered through a celite pad, and the filtered was concentrated to dryness under reduced pressure to afford 323-4 (149.7 mg, 0.208 mmol, 100%) as an off-white solid, which was used in the next step without further purification.

### Step 5

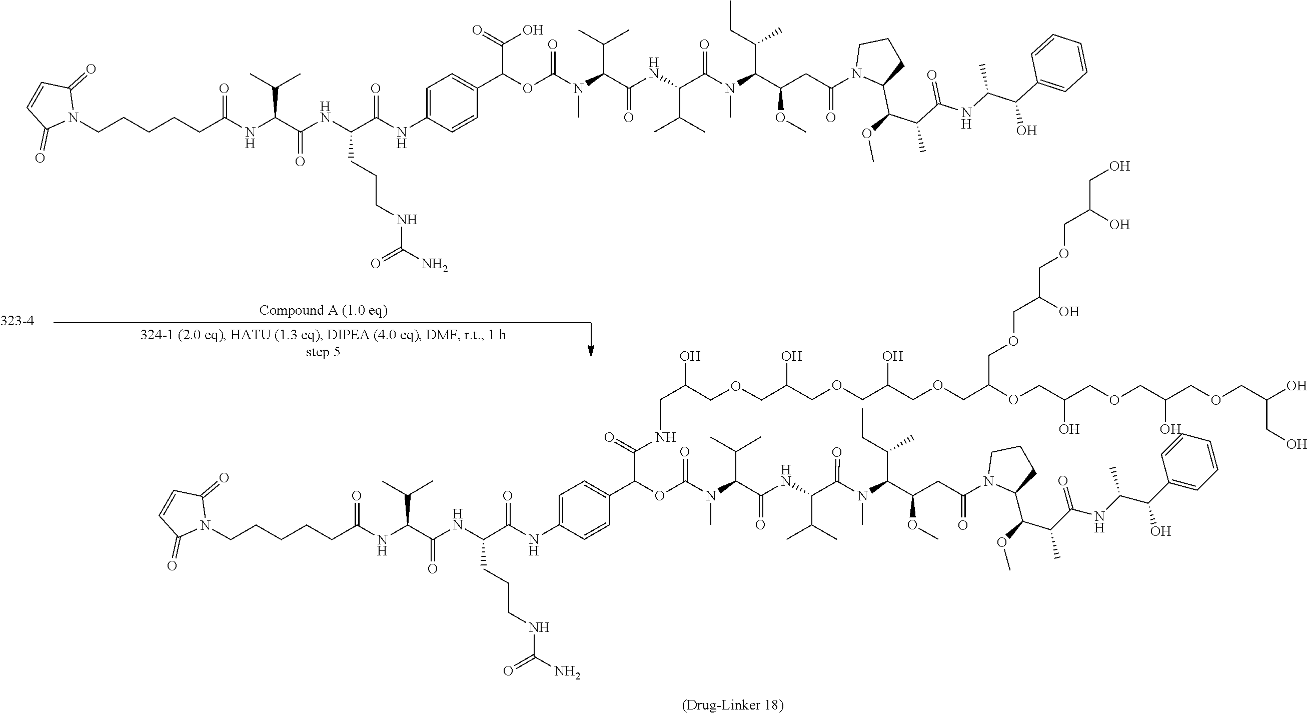

A solution of 323-4 (63.5 mg, 0.0883 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed.

Then the reaction solution was purified by prep-HPLC to give drug-linker 18 (35.0 mg, 0.0173 mmol, 39.2%) as a white solid. LCMS, m/z=1013.85 (M/2+H)⁺.

### Example 19: Preparation of Drug-Linker 19

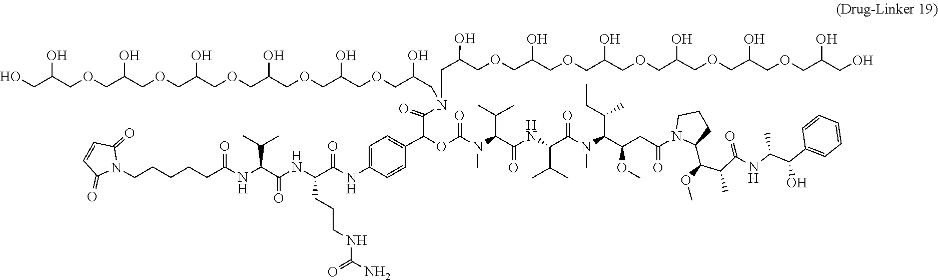 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CC(O)COCC(O)COCC(O)COCC(O)COCC(O)COCC(O)CO)CC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

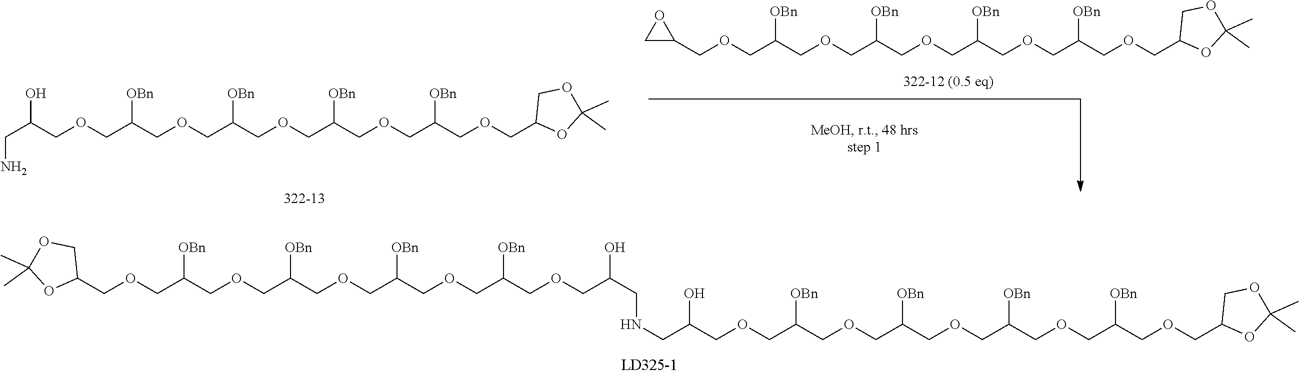

A solution of 322-13 (408 mg, 0.474 mmol), 322-12 (200 mg, 0.237 mmol) and MeOH (20 mL) was stirred at room temperature for 48 hrs. Then the reaction solution was purified by prep-HPLC to afford 325-1 (121.9 mg, 0.0715 mmol, 30.2%) as a colorless oil.

### Step 2

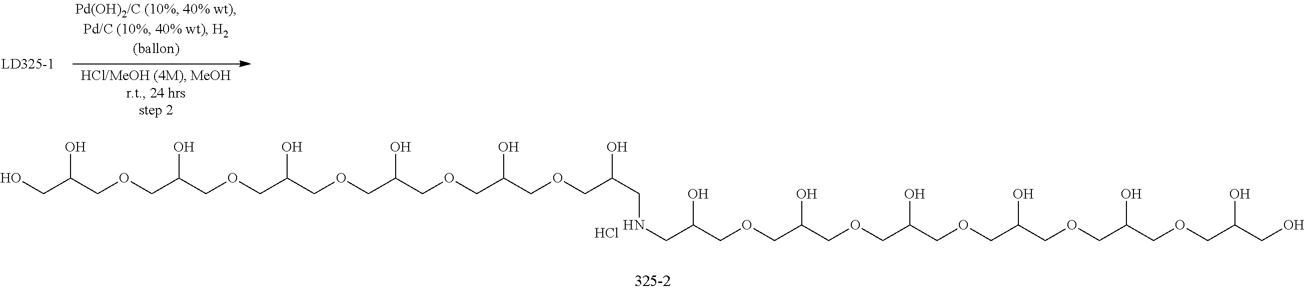 [SMILES: OCC(O)COCC(O)COCC(O)COCC(O)COCC(O)COCC(O)CNCC(O)COCC(O)COCC(O)COCC(O)COCC(O)COCC(O)CO ; Cl]

A mixture of 325-1 (121.9 mg, 0.0715 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (balloon) for 24 h at room temperature until 325-1 was completely converted into 325-2. The reaction mixture was filtered through a celite pad, and the filtrate was concentrated to dryness under reduced pressure to afford 323-4 (67.2 mg, 0.0714 mmol, 100%) as an off-white solid, which was used in the next step without further purification.

### Step 3

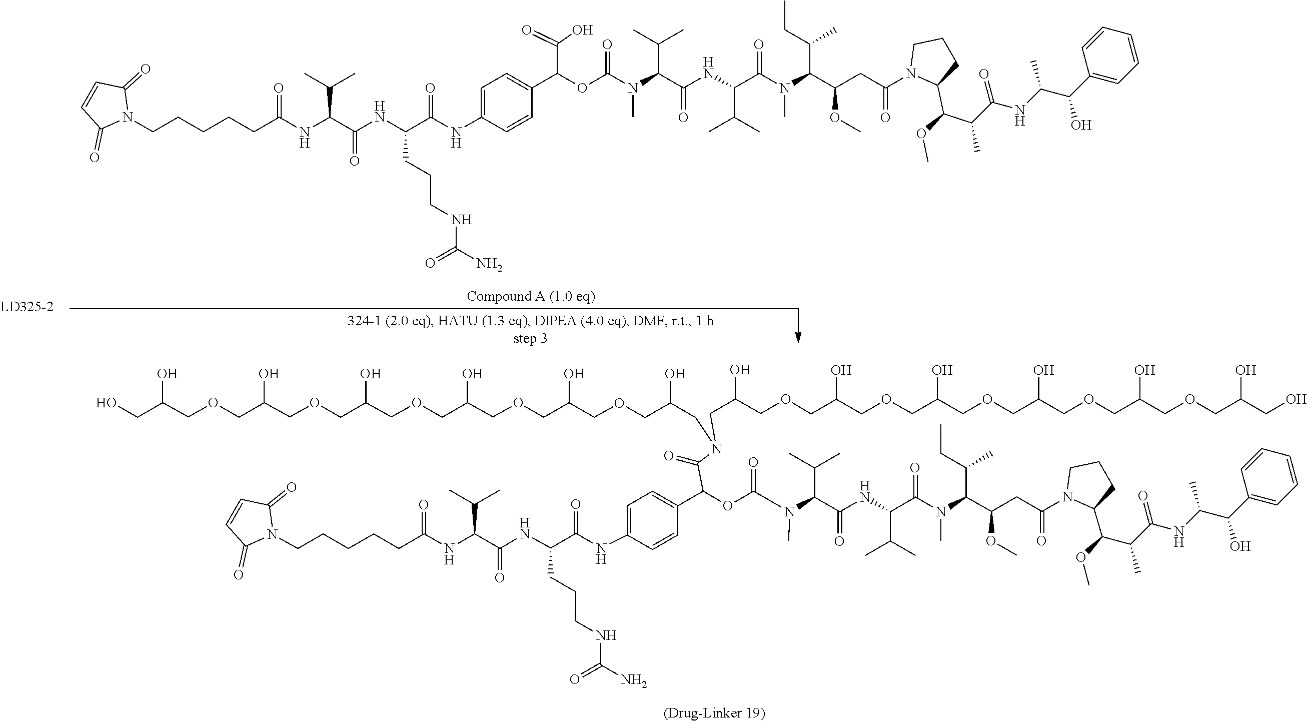

A solution of 325-2 (67.2 mg, 0.0714 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed. Then the reaction solution was purified by prep-HPLC to give drug-linker 19 (25.0 mg, 0.0111 mmol, 25.2%) as a white solid. LCMS, m/z=1124.79 (M/2+H)⁺.

### Example 20: Preparation of Drug-Linker 20

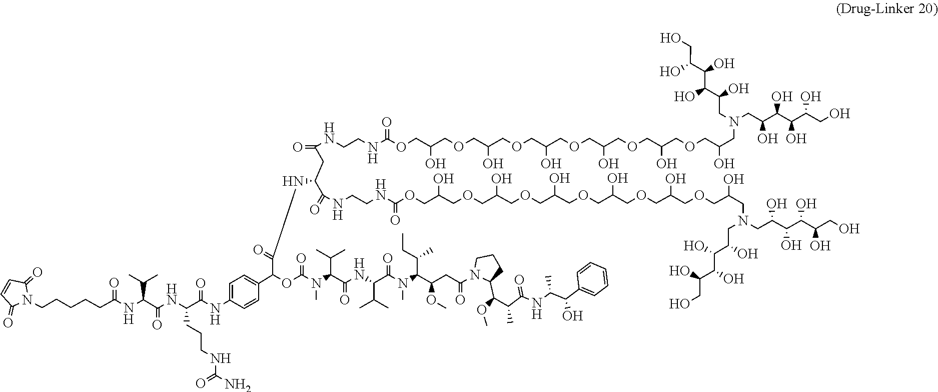 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

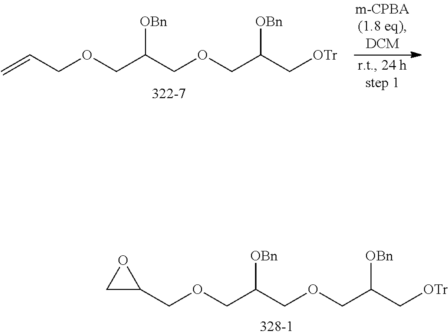

To a solution of 322-7 (10.0 g, 15.915 mmol) in DCM (20 mL) was added a solution of m-CPBA (4.94 g, 28.648 mmol) in DCM (40 mL) dropwise at room temperature. The resulting mixture was stirred at this temperature for 24 hours until 322-7 was consumed, and the reaction was quenched with saturated Na₂S₂O₃ (aq., 20 mL) and NaHCO₃ (aq, 20 mL). The reaction mixture was stirred for 30 mins, and then diluted with DCM (100 mL). The organic phase was washed with a mixture solution (20 mL×3) of saturated Na₂S₂O₃ and NaHCO₃ (aq, 1:1, VN), dried over anhydrous Na₂SO₄, filtered and concentrated to dryness under reduced pressure to give the crude product, which was purified with column chromatography (silica, 0-90% ethyl acetate in petroleum ether) affording 328-1 (9.1 g, 14.12 mmol, 88.7%) as a pale yellow oil. Purity=90%-95%. ¹H NMR (400 MHz, CDCl₃) δ 7.47-7.38 (m, 5H), 7.36-7.03 (m, 20H), 4.70-4.39 (m, 4H), 3.79-3.41 (m, 9H), 3.40-3.32 (m, 1H), 3.28-3.14 (m, 2H), 3.12-3.01 (m, 1H), 2.79-2.66 (m, 1H), 2.60-2.47 (m, 1H).

### Step 2

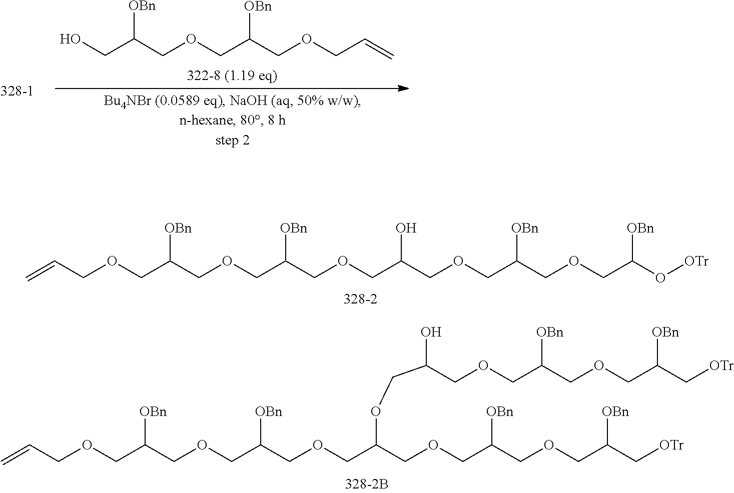

A solution of 322-8 (4.279 g, 11.08 mmol) and Bu₄NBr (177 mg, 0.548 mmol) in mixture solvent of n-hexane (20 mL) and NaOH (aq, 50% w/w, 20 mL) was stirred at 80° C. A mixture of 328-1 (6.0 g, 9.312 mmol) in n-hexane (8 mL) was dropped to the reaction mixture, and the resulting mixture was stirred at 80° C. for 8 hrs until most of 328-1 was consumed by TLC. The mixture was cooled down to r.t. and diluted with water (30 mL), then it was extracted with MTBE (70 mL×3). The organic parts were combined, dried over anhydrous Na₂SO₄, filtered and concentrated to dryness under reduced pressure. The crude product was purified with column chromatography (silica, 0-100% Ethyl acetate in petroleum ether) affording 328-2 (7.22 g, contains 328-2B) as a pale yellow oil. ¹H NMR (400 MHz, CDCl₃) δ 7.48-7.41 (m, 6H), 7.37-7.24 (m, 29H), 5.95-5.81 (m, 1H), 5.29-5.22 (m, 1H), 5.16 (dd, J=10.4, 1.7 Hz, 1H), 4.69-4.63 (m, 6H), 4.62-4.58 (m, 2H), 4.00-3.95 (m, 2H), 3.92-3.85 (m, 1H), 3.76-3.70 (m, 3H), 3.69-3.64 (m, 1H), 3.62-3.50 (m, 15H), 3.48-3.39 (m, 4H), 3.23 (d, J=5.2 Hz, 2H).

### Step 3

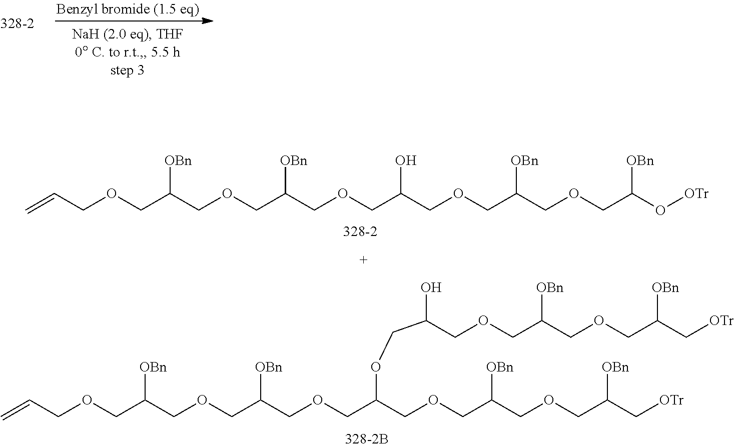

A mixture of NaH (560.5 mg, 14.012 mmol) in anhydrous THE (23.5 mL) was stirred in an ice bath, then a solution of 328-2 (7.22 g, 7.006 mmol, contains 328-2B) in anhydrous THE (13.2 mL) was added dropwise. The reaction mixture was stirred at this temperature for 20 mins, then benzyl bromide (1.786 g, 10.509 mmol) was added. The resulting mixture was allowed to warm to r.t. and stirred for 5 hrs until 328-2 was consumed by TLC. The reaction was quenched with saturated ammonium (20 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-80% Ethyl acetate in petroleum ether) affording 328-3 (5.13 g, 4.578 mmol, 65.3%) as a pale yellow oil and 328-3B (2.0 g, 1.133 mmol) a pale yellow oil. Purity=90%-95%. ¹H NMR (400 MHz, CDCl₃) δ 7.46-7.42 (m, 6H), 7.33-7.20 (m, 34H), 5.94-5.81 (m, 1H), 5.27-5.22 (m, 1H), 5.17-5.12 (m, 1H), 4.67-4.63 (m, 8H), 4.61 (s, 2H), 3.99-3.95 (m, 2H), 3.75-3.66 (m, 5H), 3.60-3.50 (m, 18H), 3.23 (d, J=5.2 Hz, 2H). ¹H NMR (400 MHz, CDCl₃) δ 7.46-7.40 (m, 13H), 7.34-7.25 (m, 27H), 7.24-7.15 (m, 25H), 5.93-5.81 (m, 1H), 5.26-5.21 (m, 1H), 5.14 (dd, J=10.4, 1.6 Hz, 1H), 4.67-4.65 (m, 2H), 4.64-4.61 (m, 8H), 4.60-4.57 (m, 4H), 3.99-3.93 (m, 2H), 3.75-3.63 (m, 10H), 3.59-3.55 (m, 6H), 3.54-3.46 (m, 20H), 3.21 (d, J=4.8 Hz, 4H).

### Step 4

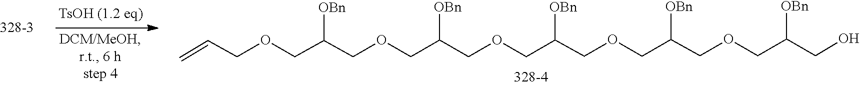 [SMILES: C=CCOCC(COCC(COCC(COCC(COCC(CO)OCc1ccccc1)OCc1ccccc1)OCc1ccccc1)OCc1ccccc1)OCc1ccccc1]

To a solution of 328-3 (3.6 g, 3.213 mmol) in a mixture solvent of DCM (22 mL) and MeOH (11 mL) was added TsOH·H₂O (733 mg, 3.855 mmol), the resulting mixture was stirred for 6 hrs at room temperature until 328-3 was consumed by TLC. The reaction was quenched with saturated NaHCO₃ (aq.), diluted with water (20 mL), and then extracted with DCM (40 mL×3). The organic phase was combined, dried over anhydrous Na₂SO₄, filtered and concentrated to dryness under reduced pressure to give the crude product, which was purified with column chromatography (silica, 0-60% ethyl acetate in petroleum ether) affording 328-4 (2.84 g, 23.233 mmol, 100%) as a colorless oil. Purity=90%-95%. ¹H NMR (400 MHz, CDCl₃) δ 7.38-7.26 (m, 25H), 5.96-5.84 (m, 1H), 5.27 (dq, J=17.2, 1.8 Hz, 1H), 5.17 (dq, J=10.4, 1.6 Hz, 1H), 4.73-4.63 (m, 10H), 4.63-4.58 (m, 1H), 3.99 (dt, J=5.6, 1.6 Hz, 2H), 3.78-3.71 (m, 5H), 3.67-3.63 (m, 2H), 3.62-3.54 (m, 18H).

### Step 5

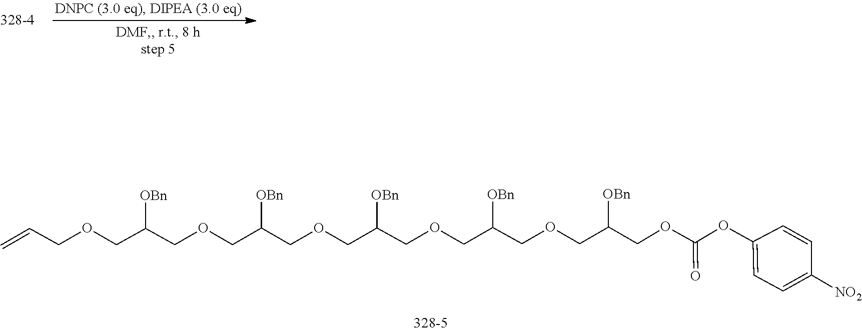 [SMILES: C=CCOCC(COCC(COCC(COCC(COCC(COC(=O)Oc1ccc([N+](=O)[O-])cc1)OCc1ccccc1)OCc1ccccc1)OCc1ccccc1)OCc1ccccc1)OCc1ccccc1]

To a solution of 328-4 (3.6 g, 3.213 mmol) in DMF (8 mL) was added DIPEA (353.2 mg, 2.733 mmol), followed by 4,4′-dinitrodiphenyl carbonate (831.1 mg, 2.732 mmol), then the resulting mixture was stirred at room temperature for 8 hrs until 328-4 was consumed detected by LCMS. The reaction solution was directly used in the next step without work-up procedure.

### Step 6

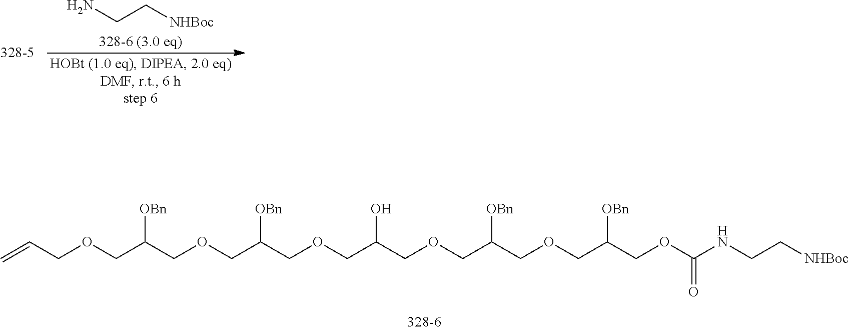 [SMILES: CC(C)(C)OC(=O)NCCN ; C=CCOCC(COCC(COCC(O)COCC(COCC(COC(=O)NCCNC(=O)OC(C)(C)C)OCc1ccccc1)OCc1ccccc1)OCc1ccccc1)OCc1ccccc1]

To the above reaction mixture was added HOBt (123 mg, 0.911 mmol), DIPEA (235.5 mg, 1.822 mmol) and 328-6 (437.6 mg, 2.733 mmol) successively, and the resulting mixture was stirred at room temperature for 6 hrs until 328-5 was consumed detected by LCMS. The reaction mixture was diluted with ethyl acetate (100 mL) and washed with saturated NaHCO₃ (aq, 30 mL×3), dried over anhydrous Na₂SO₄, filtered and concentrated to dryness under reduced pressure. The crude product was purified with column chromatography (silica, 0-90% ethyl acetate in petroleum ether) affording 328-6 (958 mg, 0.90 mmol, 98.7% over 2 steps) as a pale yellow oil. ¹H NMR (400 MHz, CDCl₃) δ 7.36-7.22 (m, 25H), 5.95-5.80 (m, 1H), 5.28-5.21 (m, 1H), 5.20-5.13 (m, 1H), 5.07 (s, 1H), 4.83 (s, 1H), 4.69-4.62 (m, 10H), 4.30-4.21 (m, 1H), 4.16-4.10 (m, 1H), 3.97 (dt, J=5.6, 1.6 Hz, 2H), 3.77-3.69 (m, 5H), 3.62-3.50 (m, 19H), 3.27-3.10 (m, 4H), 1.43 (s, 9H).

### Step 7

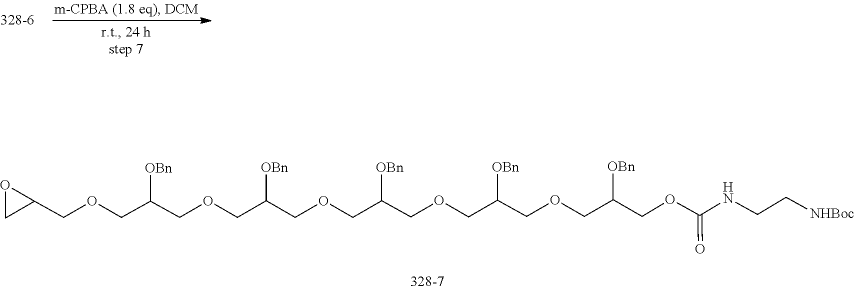 [SMILES: CC(C)(C)OC(=O)NCCNC(=O)OCC(COCC(COCC(COCC(COCC(COCC1CO1)OCc1ccccc1)OCc1ccccc1)OCc1ccccc1)OCc1ccccc1)OCc1ccccc1]

To a solution of 328-6 (958 mg, 0.90 mmol) in DCM (6 mL) was added a solution of m-CPBA (280 mg, 1.62 mmol) in DCM (6 mL) dropwise at room temperature. The resulting mixture was stirred at this temperature for 24 hours until 328-6 was consumed, and the reaction was quenched with saturated Na₂S₂O₃ (aq, 5 mL) and NaHCO₃ (aq., 5 mL). The reaction mixture was stirred for 30 mins, and then diluted with DCM (30 mL). The organic phase was washed with a mixture solution (10 mL×3) of saturated Na₂S₂O₃ and NaHCO₃(aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 328-7 (787 mg, 0.728 mmol, 80.9%) as a pale yellow oil. Purity=90%-95%.

### Step 8

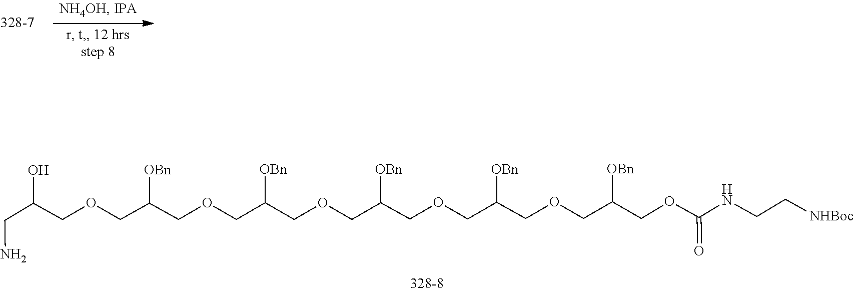 [SMILES: CC(C)(C)OC(=O)NCCNC(=O)OCC(COCC(COCC(COCC(COCC(COCC(O)CN)OCc1ccccc1)OCc1ccccc1)OCc1ccccc1)OCc1ccccc1)OCc1ccccc1]

To a solution of 328-7 (565 mg, 0.523 mmol) in isopropyl alcohol (43 mL) was added ammonia (43 mL) dropwise at room temperature, and the resulting mixture was stirred at this temperature for 12 hours until 328-7 was consumed. The reaction mixture was concentrated to dryness under reduced pressure to afford 328-8 (552.6 mg, 0.503 mmol, 96.3%) as a yellow oil, which was used in the next step without further purification. Purity=90%-95%.

### Step 9

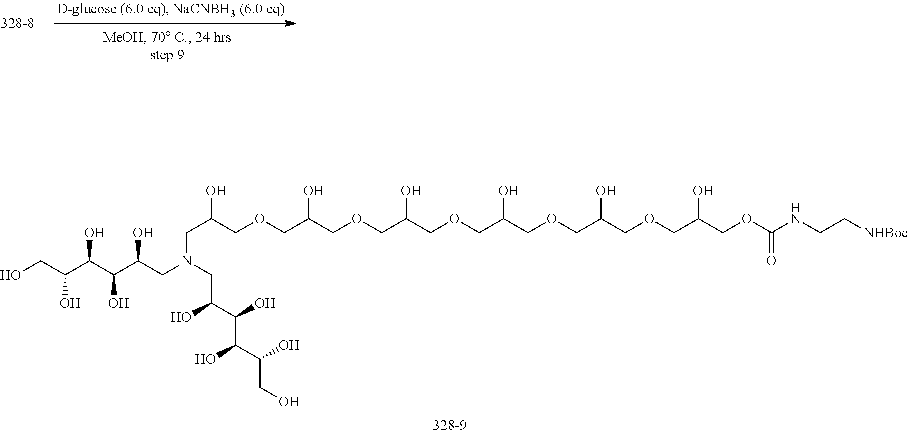 [SMILES: CC(C)(C)OC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 328-8 (552.6 mg, 0.503 mmol), D-glucose (544.2 mg, 3.021 mmol) and NaCNBH₃ (189.8 mg, 3.021 mmol) in anhydrous MeOH (12 mL) was stirred at 70° C. for 24 hrs until most of 328-8 was consumed and 328-9 was detected by LCMS. The reaction mixture was cooled down to room temperature, filtered and concentrated under reduced pressure to give the crude product, which was purified by reverse phase liquid chromatography to give 328-9 (597 mg, 0.419 mmol, 83.2%) as a colorless oil. Purity=90%-95%. ¹H NMR (400 MHz, MeOH-d₄) δ 7.41-7.20 (m, 25H), 4.69-4.57 (m, 10H), 4.26-4.04 (m, 5H), 3.84-3.78 (m, 3H), 3.77-3.68 (m, 8H), 3.67-3.42 (m, 28H), 3.38-3.34 (m, 1H), 3.33-3.31 (m, 1H), 3.17-3.06 (m, 4H), 1.40 (s, 9H).

### Step 10

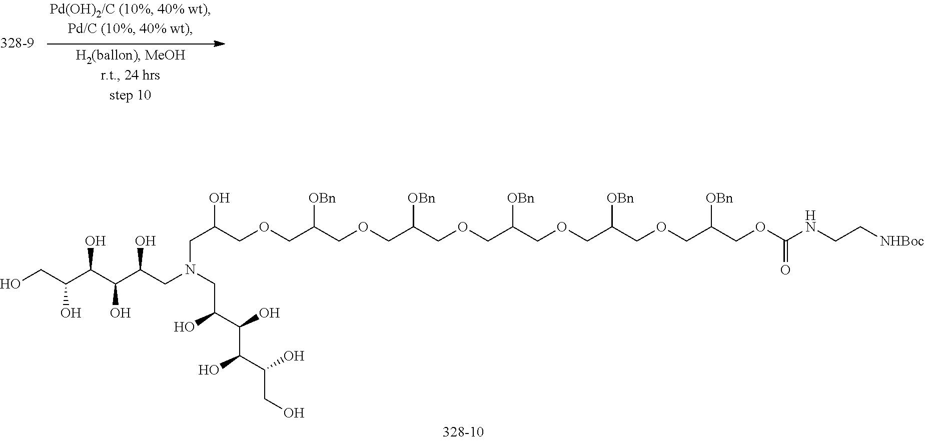 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-9 (376 mg, 0.264 mmol), Pd(OH)₂/C (10%, 150 mg) and Pd/C (10%, 150 mg) in MeOH (30 mL) was stirred under hydrogen atmosphere (balloon) for 24 h at room temperature until 328-9 was completely converted into 328-10. The reaction mixture was filtered through a celite pad, and the filtrate was concentrated to dryness under reduced pressure to afford 328-10 (211.4 mg, 0.217 mmol, 82.1%) as an off-white solid, which was used in the next step without further purification.

### Step 11

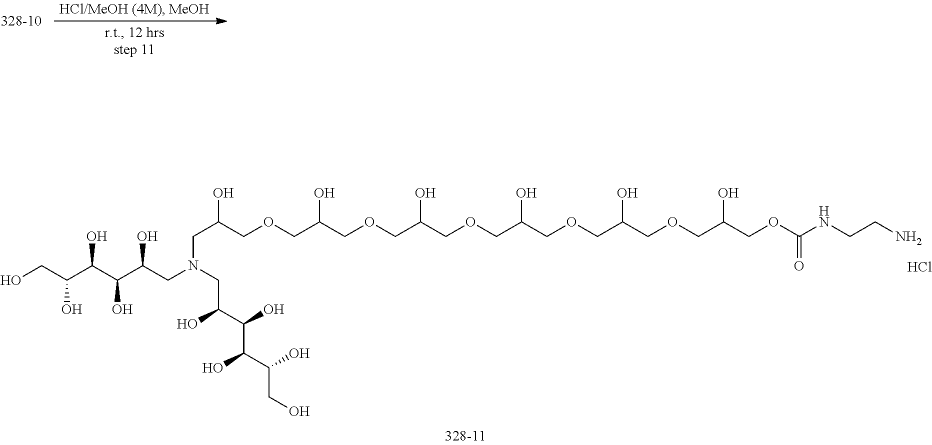 [SMILES: NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 328-10 (211.4 mg, 0.216 mmol) in HCl/MeOH (4M, 3 mL) and MeOH (3 mL) was stirred at room temperature for 12 hours until 328-10 was completely converted into 328-11. The reaction mixture was concentrated to dryness under reduced pressure to afford 328-11 (198.0 mg, 0.217 mmol, 100%) as an off-white solid, which was used in the next step without further purification.

### Step 12

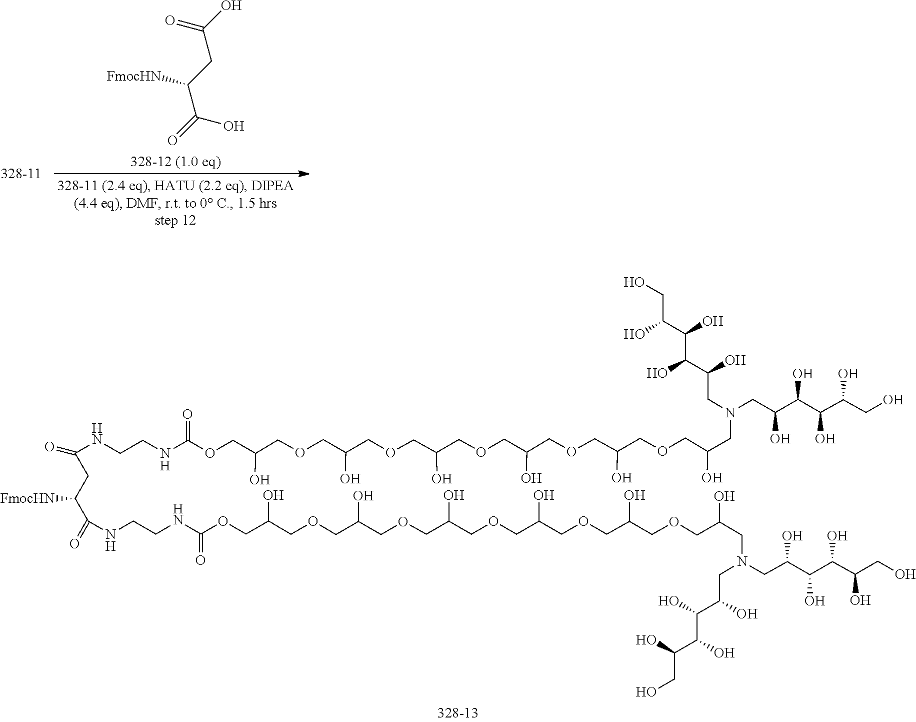

A solution of 328-12 (24.8 mg, 0.0698 mmol) and HATU (58.4 mg, 0.153 mmol) in anhydrous DMF (2 mL) was stirred at room temperature for 15 mins, then it was stirred in an ice bath. A solution of 328-11 (150 mg, 0.168 mmol) in anhydrous DMF (2 mL) was added dropwise, followed by DIPEA (39.7 mg, 0.307 mmol). The resulting mixture was stirred in the ice bath for 1 h until most of 328-12 was consumed. The reaction mixture was purified by reverse phase liquid chromatography to give 328-13 (97.5 mg, 0.0471 mmol, 67.4%) as a colorless oil. Purity=90%-95%.

### Step 13

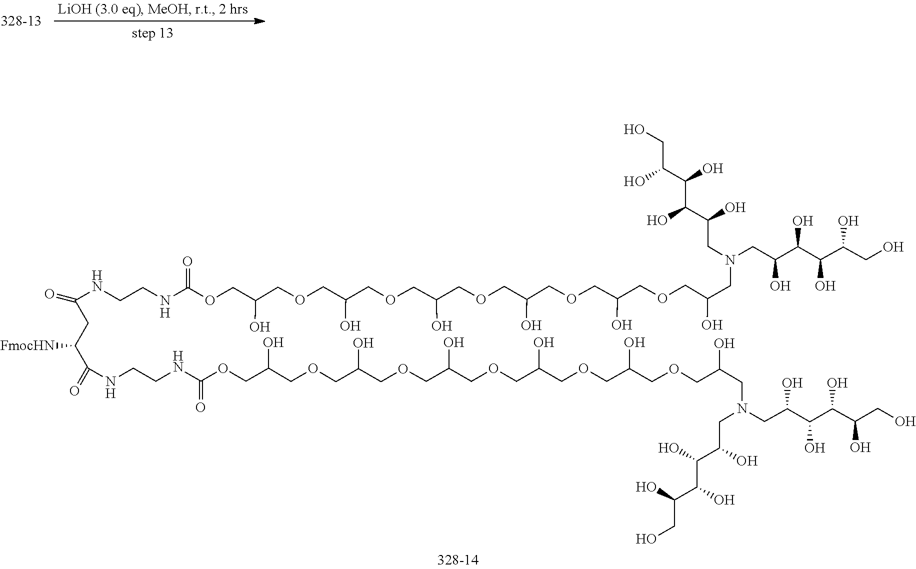

To a solution of 328-13 (97.5 mg, 0.0471 mmol) in MeOH (3 mL) was added LiOH·H₂O (5.9 mg, 0.141 mmol), and the mixture was stirred at room temperature for 2 hrs until 328-13 was consumed. The reaction solution was neutralized with 1 N HCl to pH=7, and concentrated under reduced pressure to give a crude product, which was dissolved in H₂O (10 mL) and washed with hexane (5 mL×3). The aqueous phase was concentrated to dryness under reduced pressure to afford 328-14 (87.0 mg, 0.047 mmol, 100%) as a colorless oil, which was used in the next step without further purification.

### Step 14

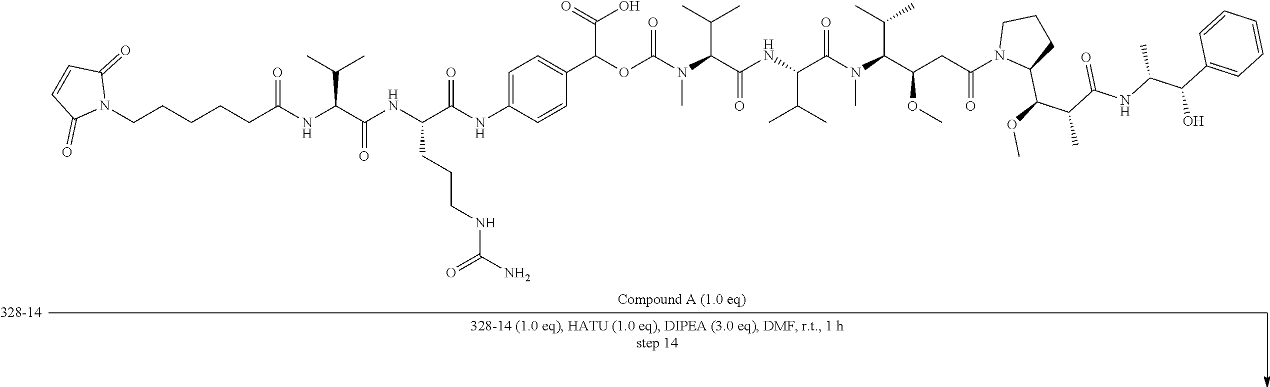

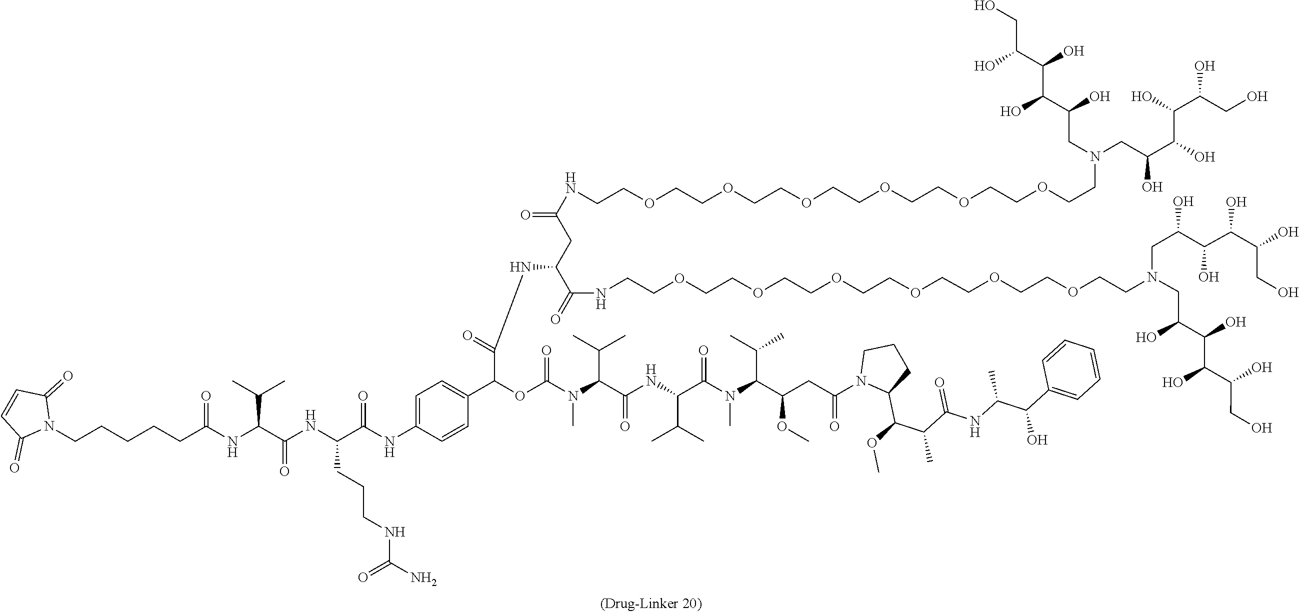

A solution of 328-14 (85 mg, 0.0460 mmol), Compound A (62 mg, 0.0460 mmol), HATU (17.2 mg, 0.0452 mmol) and DIPEA (17.9 mg, 0.139 mmol) in anhydrous DMF (4 mL) was stirred at room temperature for 1 h until Compound A was consumed. Then the reaction solution was purified by prep-HPLC to give drug-linker 20 (73.0 mg, 0.0229 mmol, 49.8%) as a white solid. LCMS, m/z=1595.97 (M/2+H)⁺, m/z=1064.29 (M/2+H)⁺. ¹H NMR (400 MHz, DMSO-d₆) δ 10.04 (s, 1H), 8.59-8.02 (m, 4H), 8.00-7.72 (m, 2H), 7.70-7.46 (m, 3H), 7.45-7.09 (m, 8H), 7.00 (s, 2H), 5.99 (s, 1H), 5.91-5.66 (m, 2H), 5.44 (s, 5H), 5.07-4.29 (m, 16H), 4.25-3.87 (m, 12H), 3.86-3.47 (m, 31H), 3.30-2.74 (m, 27H), 2.44-1.84 (m, 10H), 1.83-1.63 (m, 4H), 1.59-1.27 (m, 10H), 1.24-1.10 (m, 3H), 1.08-0.64 (m, 30H).

### Example 21: Preparation of Drug-Linker 21

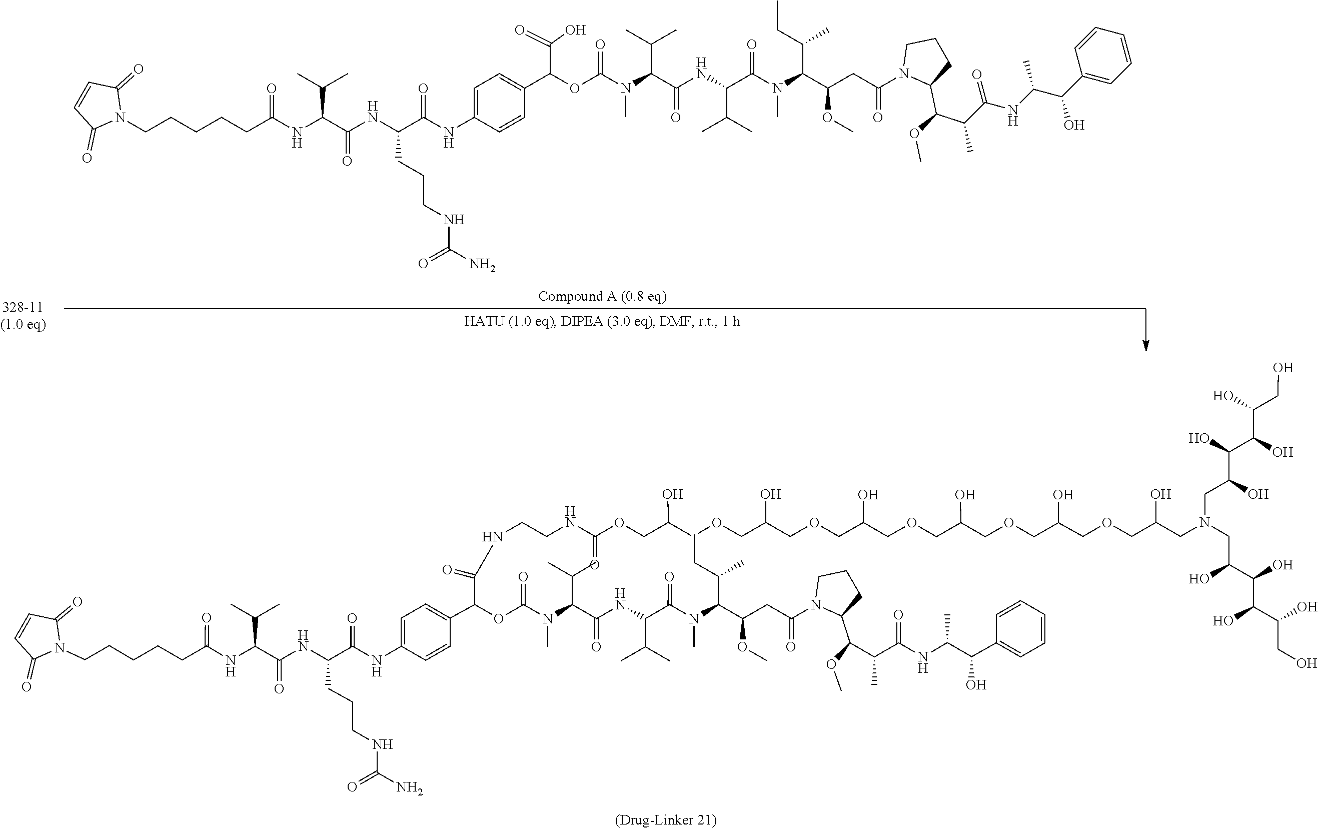

A solution of 328-11 (42 mg, 0.0461 mmol), Compound A (50 mg, 0.0368 mmol), HATU (17.4 mg, 0.0458 mmol) and DIPEA (17.8 mg, 0.138 mmol) in anhydrous DMF (3 mL) was stirred at room temperature for 1 h until Compound A was consumed. Then the reaction solution was purified by prep-HPLC to give drug-linker 21 (37.8 mg, 0.0170 mmol, 46.3%) as a white solid. LCMS, m/z=1109.75 (M/2+H)⁺. ¹H NMR (400 MHz, D₂O) δ 7.60-7.43 (m, 4H), 7.39-7.27 (m, 4H), 7.25-7.14 (m, 1H), 6.78 (s, 2H), 6.05-5.89 (m, 1H), 4.78-4.54 (m, 6H), 4.52-3.90 (m, 28H), 3.89-3.71 (m, 13H), 3.69-3.38 (m, 64H), 3.37-2.85 (m, 25H), 2.81-2.60 (m, 3H), 2.58-2.40 (m, 2H), 2.37-2.11 (m, 4H), 2.08-1.96 (m, 2H), 1.94-1.70 (m, 5H), 1.66-1.45 (m, 8H), 1.35-1.12 (m, 8H), 1.10-0.73 (m, 25H), 0.67-0.50 (m, 1H), 0.47-0.35 (m, 1H).

### Example 22: Preparation of Drug-Linker 22

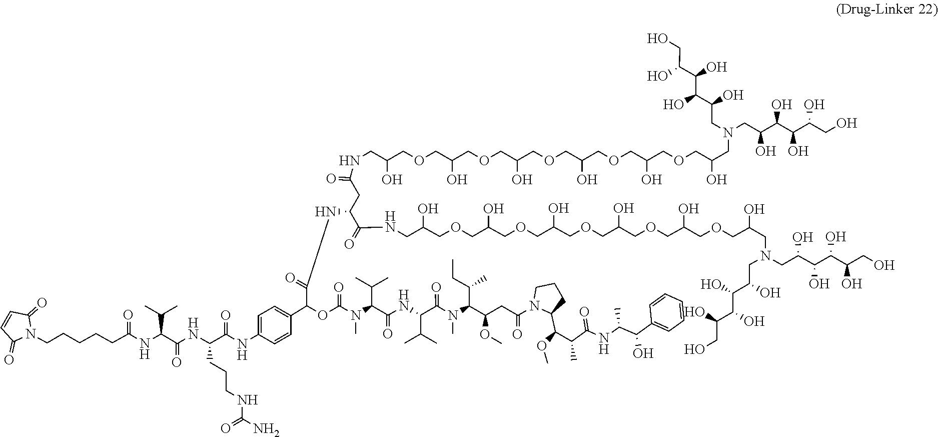 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

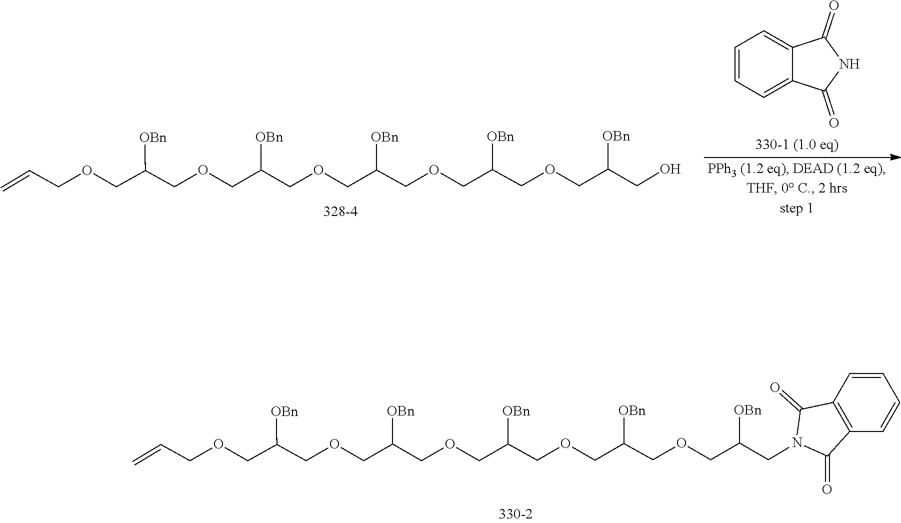 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THE (8 mL) was added a solution of 328-4 (1.3 g, 1.48 mmol) in THE (4 mL) and the mixture was stirred in an ice bath. A solution of DEAD (309.3 mg, 1.776 mmol) in THE (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. ¹H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

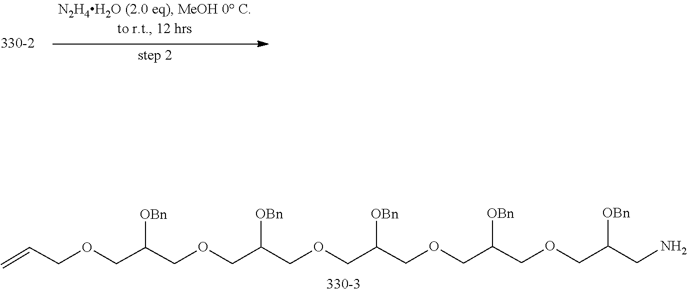 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

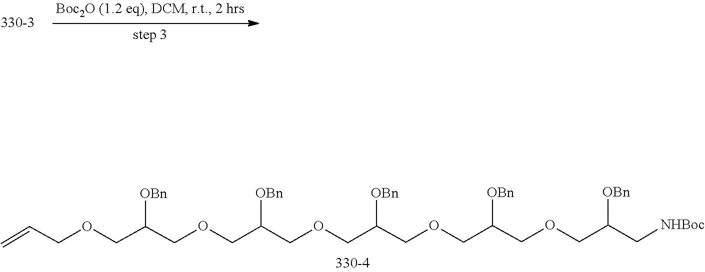 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

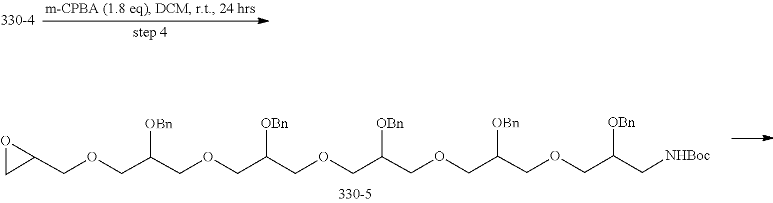 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequencely washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

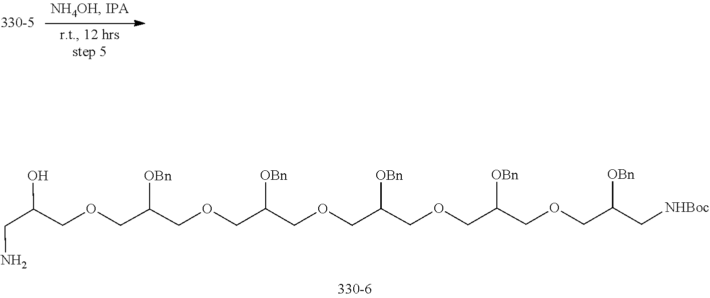 [SMILES: CC(C)(C)OC(=O)NCC(COCC(COCC(COCC(COCC(COCC(O)CN)OCc1ccccc1)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

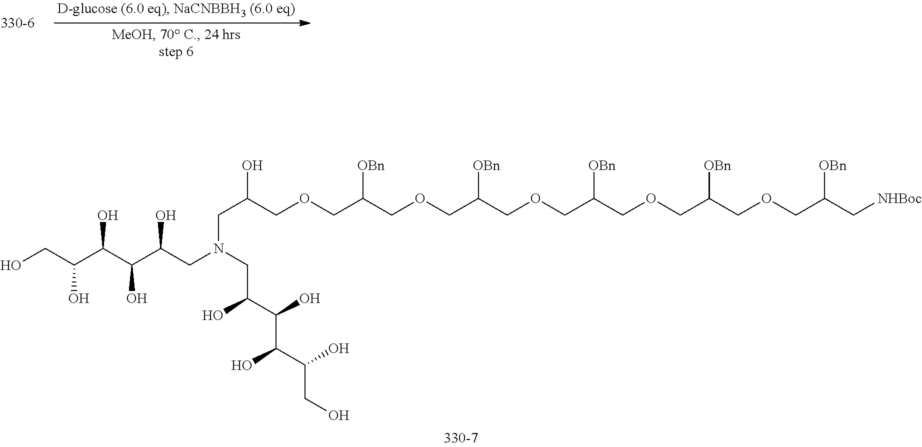 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

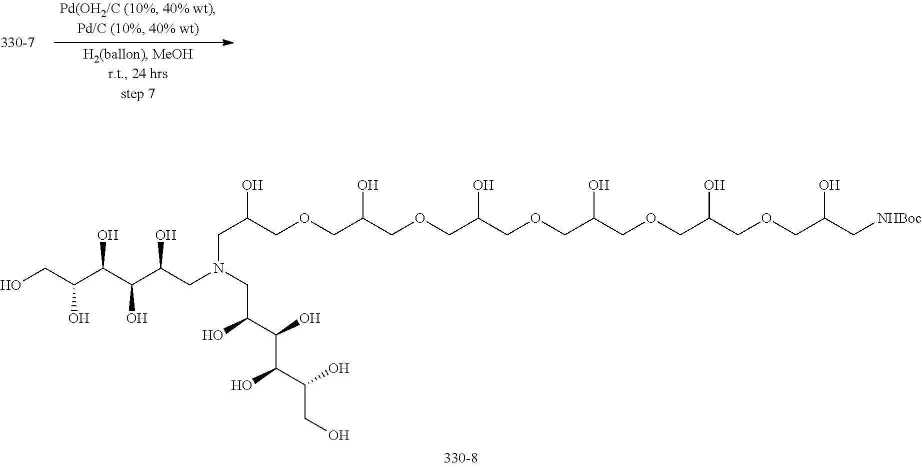 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (balloon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

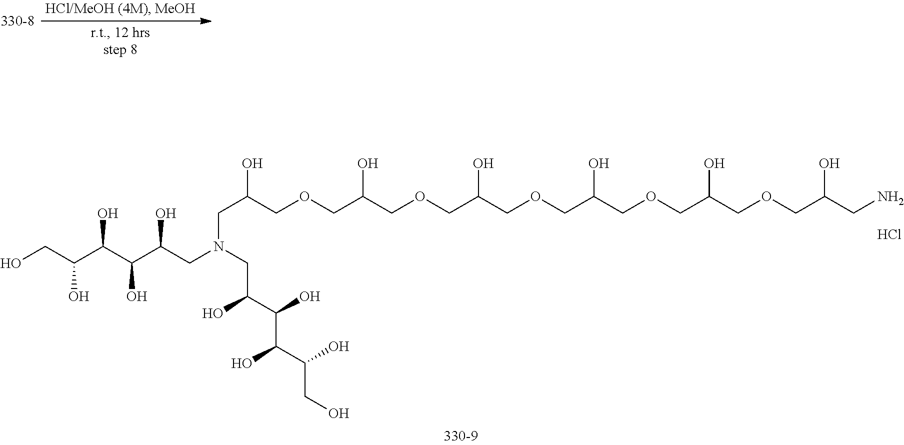 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

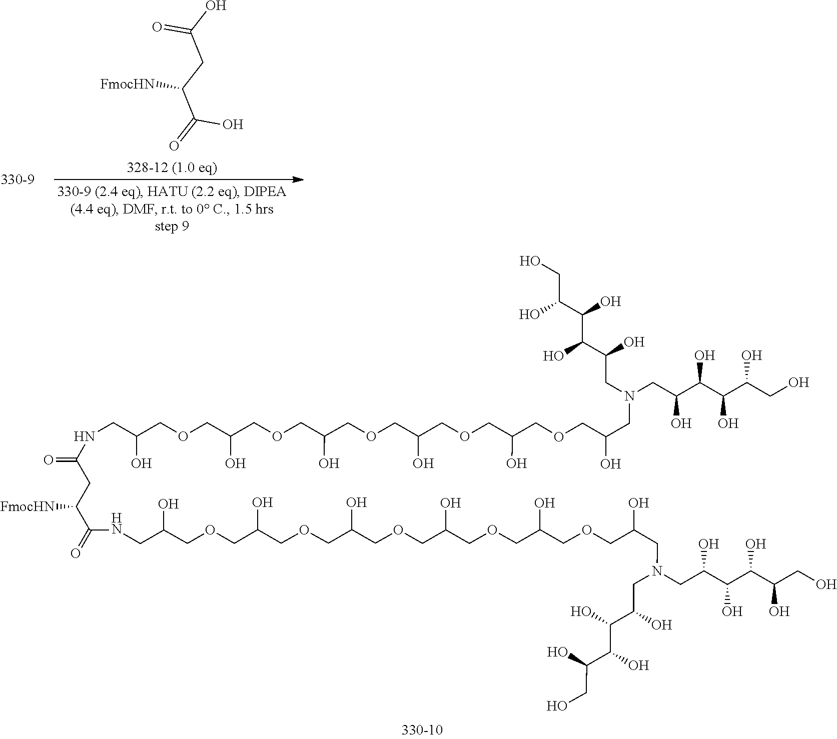

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 10

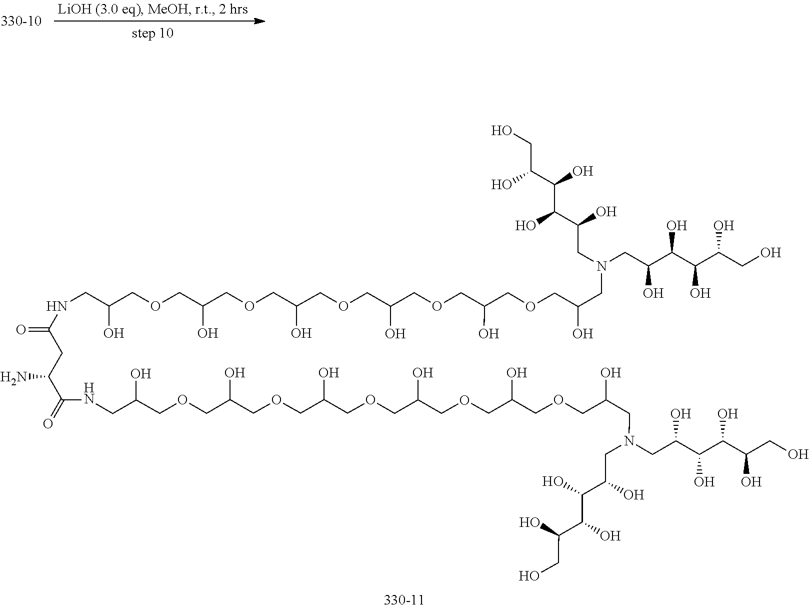 [SMILES: N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 11

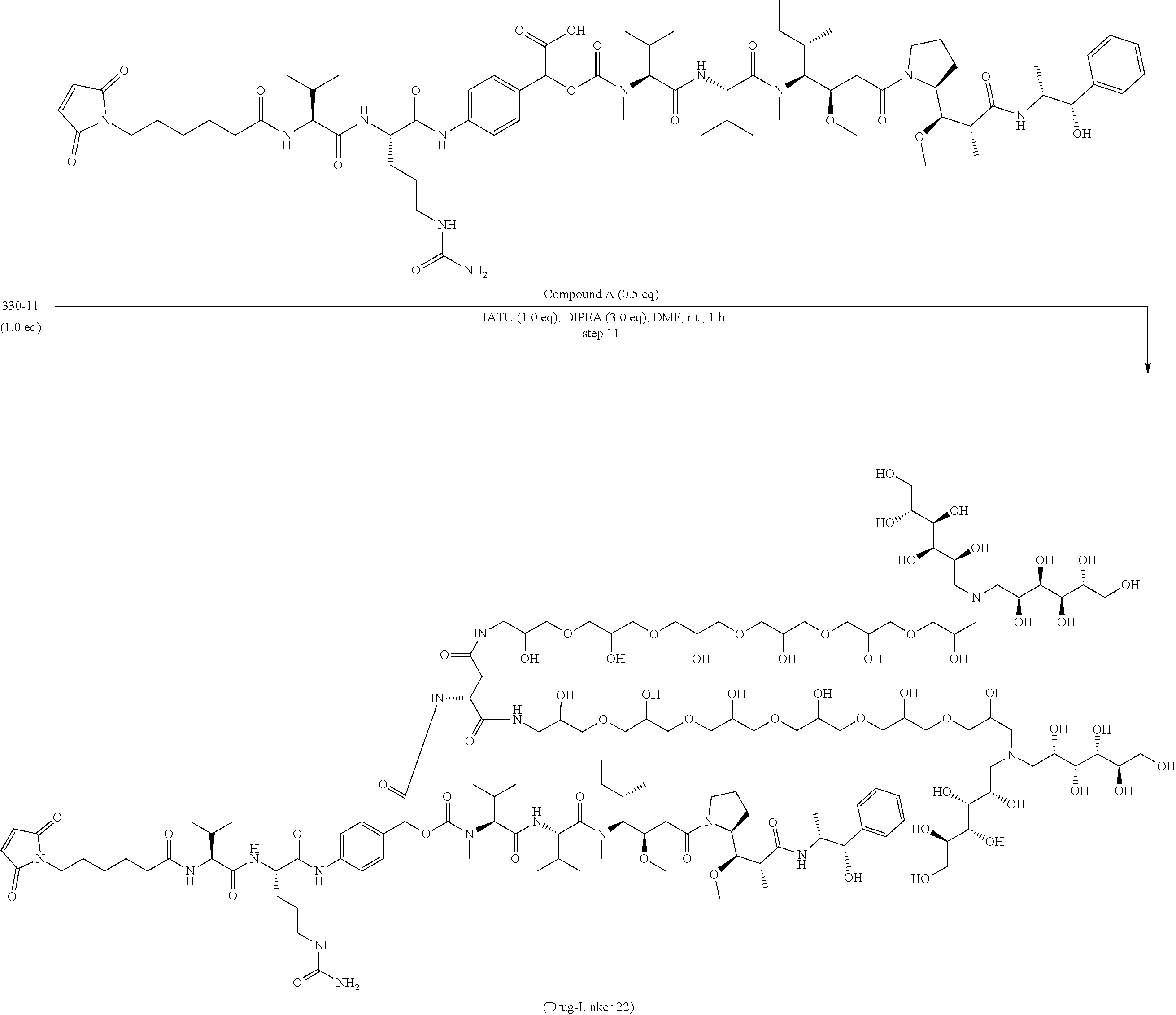

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give drug-linker 22 (5.0 mg, 0.00166 mmol, 45.1%) as a white solid. LCMS, m/z=1509.00 (M/2+H)⁺, m/z=1006.6 (M/3+H)⁺.

### Example 23: Preparation of Drug-Linker 23

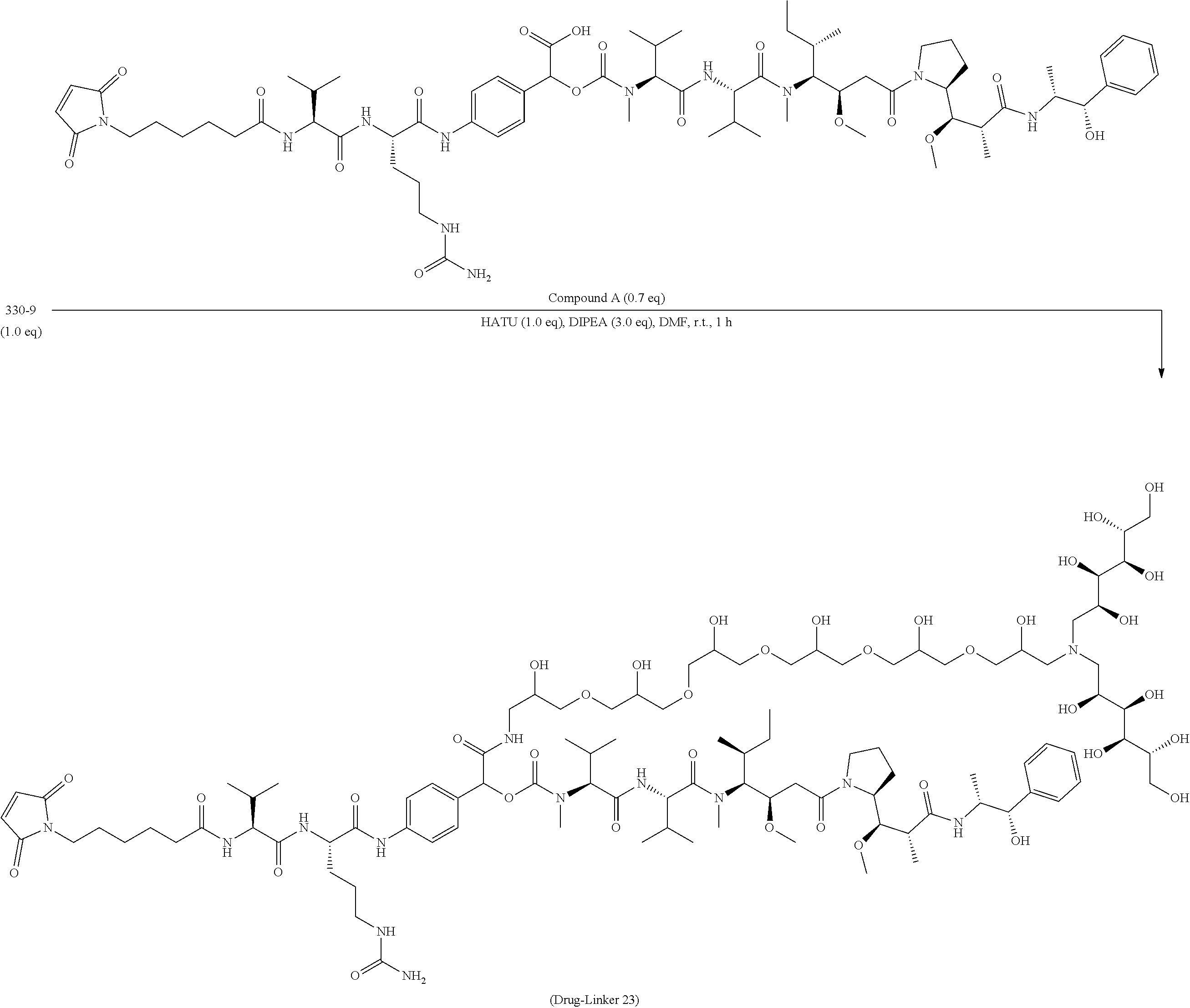

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give drug-linker 23 (28 mg, 0.0131 mmol. 33.7%) as a white solid. LCMS, m/z=1066.11 (M/2+H)⁺. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 24: Preparation of Drug-Linker 24

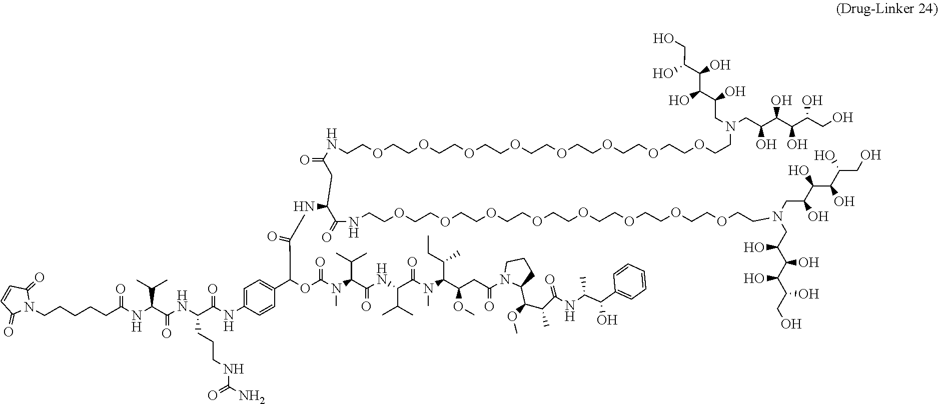 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

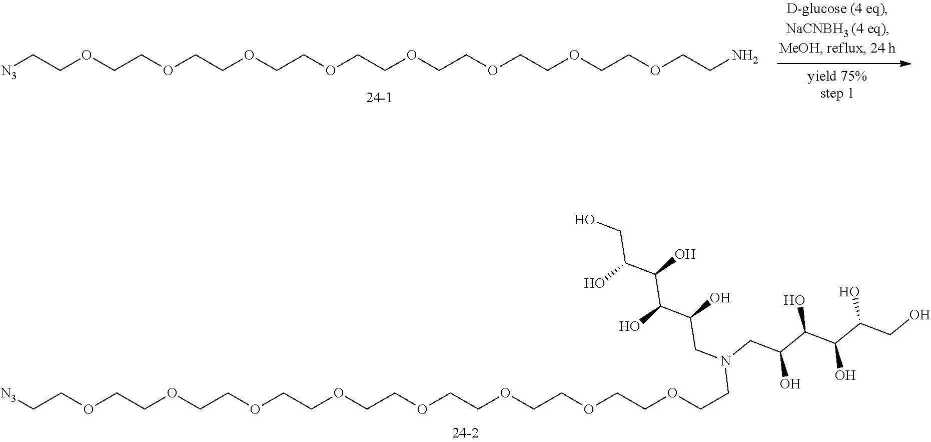 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-1 (2.5 g, 5.701 mmol) in MeOH (20 mL) was added D-Glucose (4.11 g, 22.804 mmol) and NaBH3CN (1.385 mL, 22.804 mmol). The mixture was stirred at reflux for 24 h to complete. Then the resulting solution concentrated to dryness and the residue was purified by reverse phase chromatography (C₈ column, eluting with 0-45% methanol in water with 0.01% TFA) to afford the product 24-2 as yellow oil. ESI m/z: 767.5 (M+H)⁺.

### Step 2

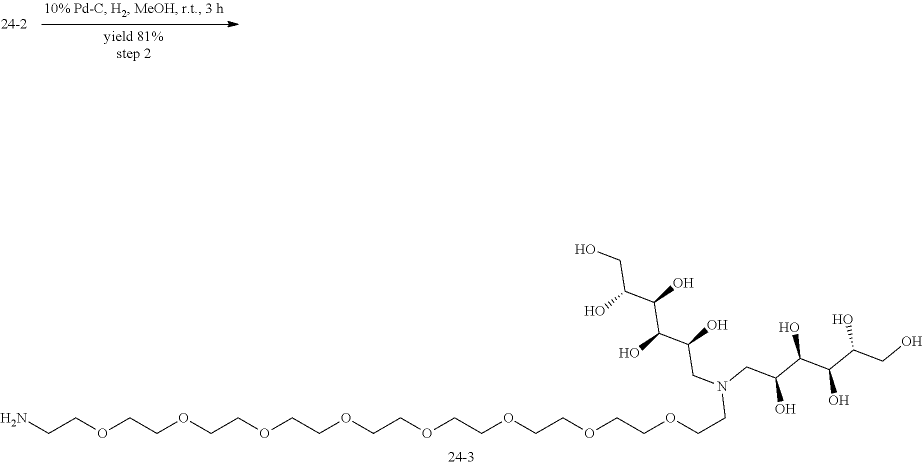 [SMILES: NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3 h to complete. The resulting mixture was filtered to remove catalyst solid and the filtrate was concentrated, then purified by reverse phase chromatography (C8 column, eluting with 0-25% acetonitrile in water with 0.01% TFA) to afford the product 24-3 (2.6 g, 3.510 mmol, 81.50%) as colorless oil. ESI m/z: 371.3 (M/2+H)+, 741.4 (M+H)⁺.

### Step 3

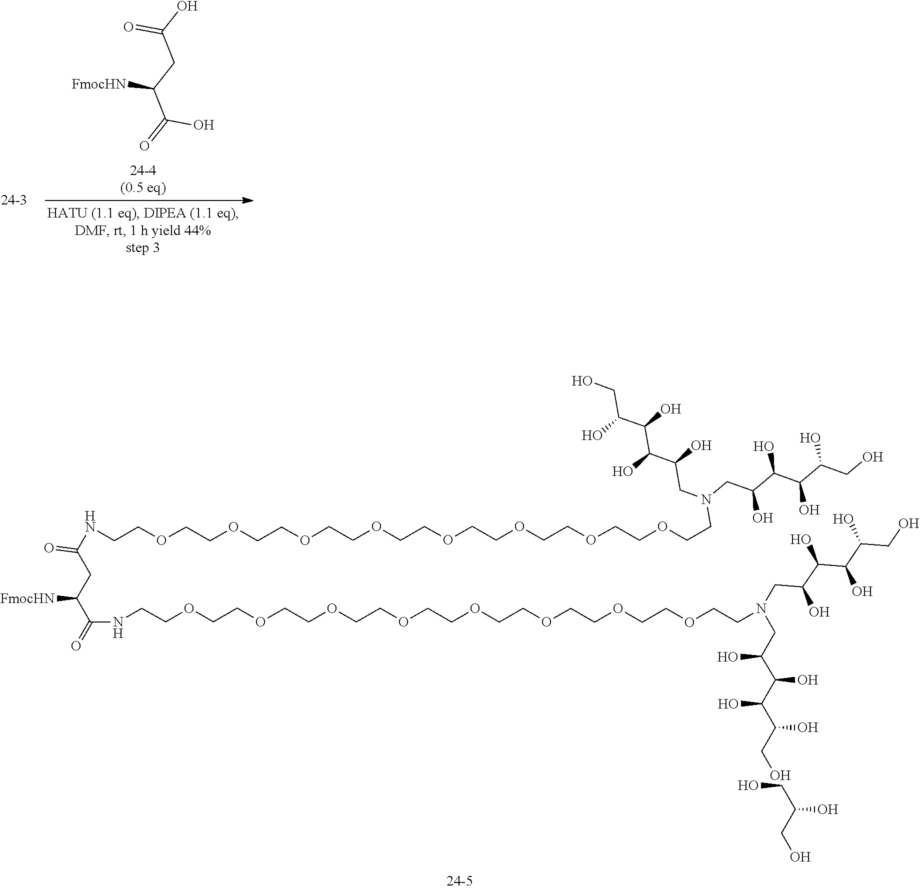

A solution of 24-4 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 24-3 (2.6 g, 3.510 mmol) in DMF (5 mL). After addition, the solution was stirred at room temperature for another 1 h to complete. The completed solution was then purified directly by reverse phase chromatography (C8 column, eluting with 0-40% acetonitrile in water with 0.01% TFA) to afford the product 24-5 (1.4 g, 0.777 mmol, 44.30%) as colorless oil. ESI m/z: 601.0 (M/3+H)⁺, 901.0 (M/2+H)⁺.

### Step 4

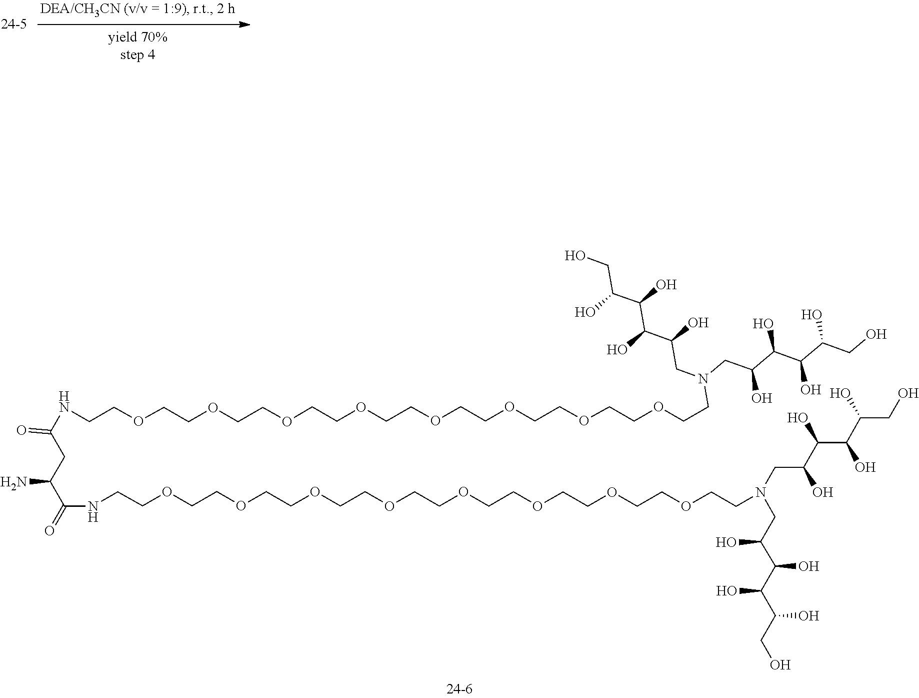

To the solution of 24-5 (1.4 g, 0.777 mmol) in MeCN (6 mL) was diethyl amine (0.7 mL, 8.930 mmol). The mixture was stirred at room temperature for 2 h to achieve complete deprotection. Then the resulting solution was concentrated under reduced pressure to remove most of diethyl amine, and the residue was purified by reverse phase chromatography (C₈ column, eluting with 0-20% acetonitrile in water with 0.01% TFA) to get desired fractions, which was freeze-dried to afford the product 24-6 (0.86 g, 0.545 mmol, 69.92%) as sticky colorless oil. ESI m/z: 526.9 (M/3+H)⁺, 789.9 (M/2+H)⁺. 1HNMR (400 MHz, DMSO-d₆) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

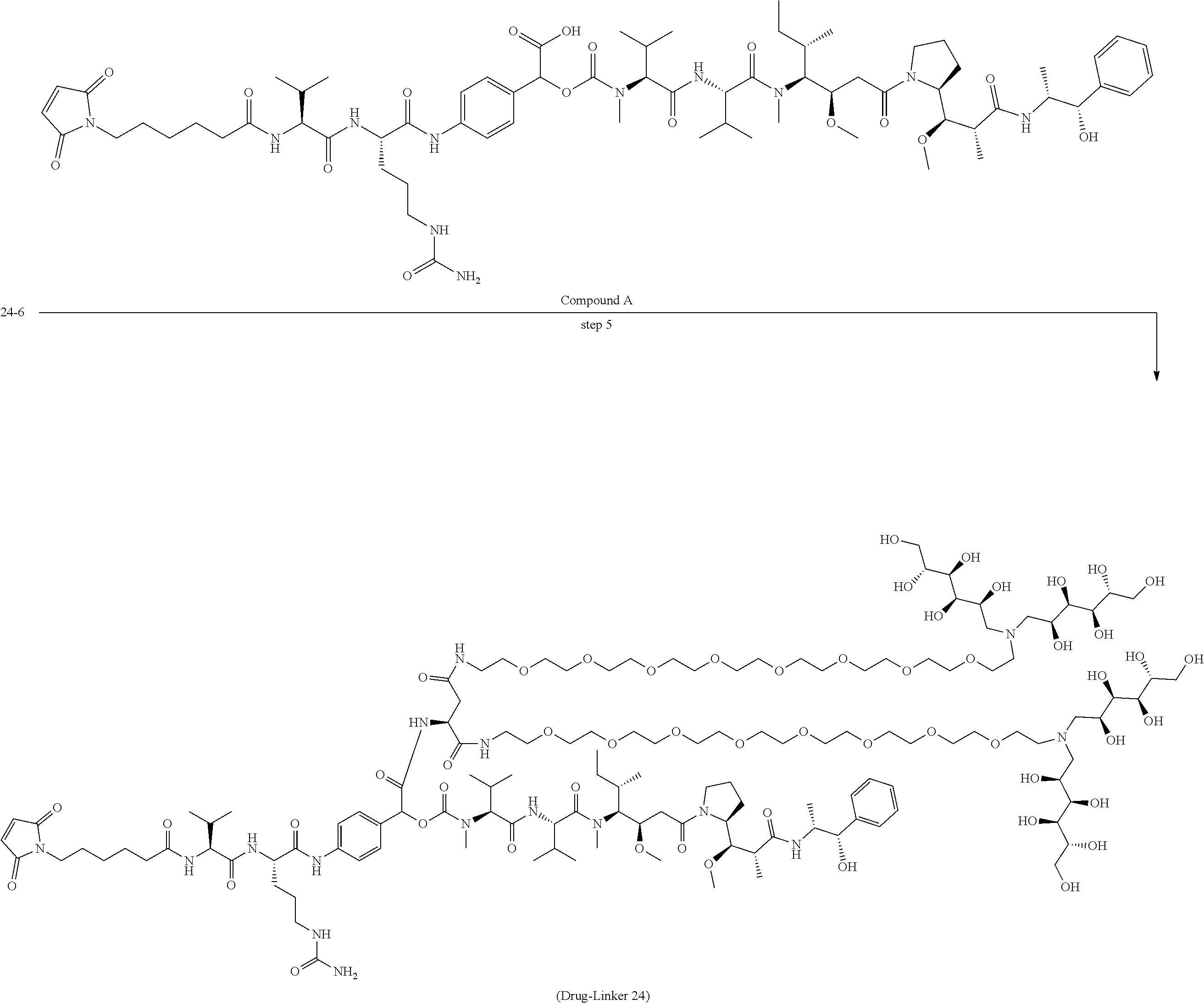

A solution of 24-6 (100 mg, 0.063 mmol), Compound A (86 mg, 0.063 mmol) and HATU (24 mg, 0.063 mmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give drug-linker 24 (40 mg, 0.014 mmol, 21.73%) as a white solid. LCMS, m/z=1461.23 (M/2+H)⁺, ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 10H), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 10H), 1.24-1.08 (m, 9H), 1.02-1.01 (m, 2H), 0.92-0.75 (m, 28H) ppm.

### Example 25: Preparation on Drug-Linker 25

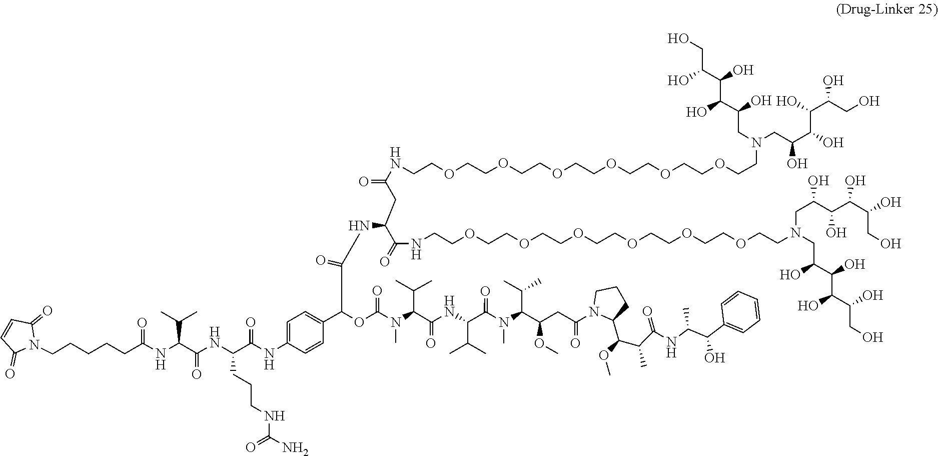

### Step 1

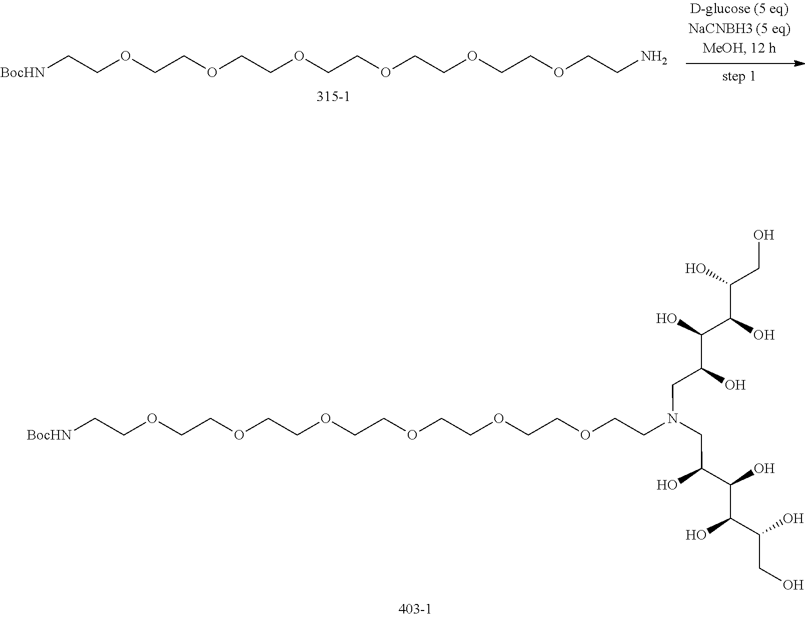 [SMILES: CC(C)(C)OC(=O)NCCOCCOCCOCCOCCOCCOCCN ; CC(C)(C)OC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-1 (1.0 g, 2.355 mmol) and D-Glucose (2.1 g, 11.656 mmol) in anhydrous Methanol (40 mL) was heated at 50° C. for 30 min, then NaCNBH₃ (740 mg, 11.776 mmol) was added. The resulting solution was stirred for another 12 hr at 70° C. until indicated all starting amine was disappeared and the mass of desired product was detected. Then the reaction solution was concentrated and purified by reverse phase liquid chromatography to give 403-1 (730 mg, 0.970 mmol, 41.17%) as a white solid.

### Step 2

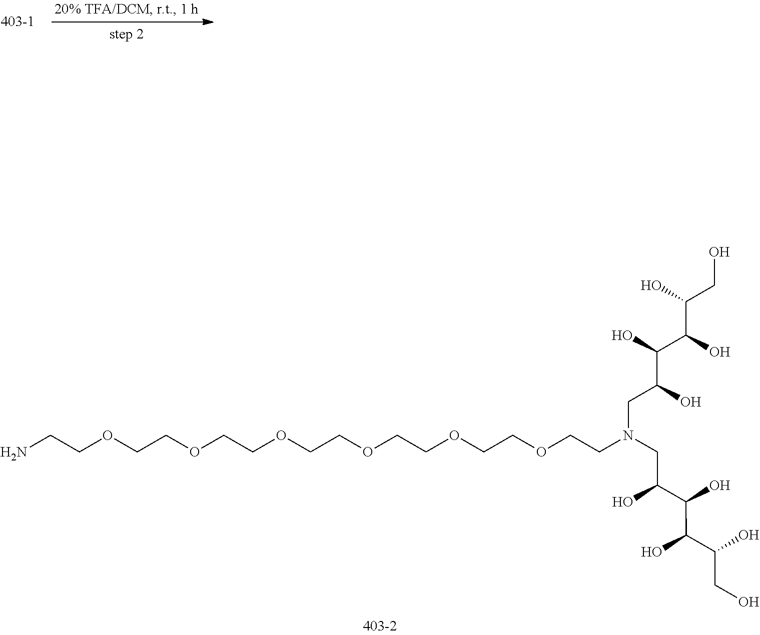 [SMILES: NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-1 (730 mg, 0.970 mmol) and TFA (2 mL) in anhydrous DCM (8 mL) was stirred at room temperature for 1 hr until LCMS indicated all starting amine was disappeared and desired product was detected. Then the solution was concentrated to dryness to 403-2 (610 mg, 0.935 mmol, 96.39%) as yellow oil, used as such in the next step.

### Step 3

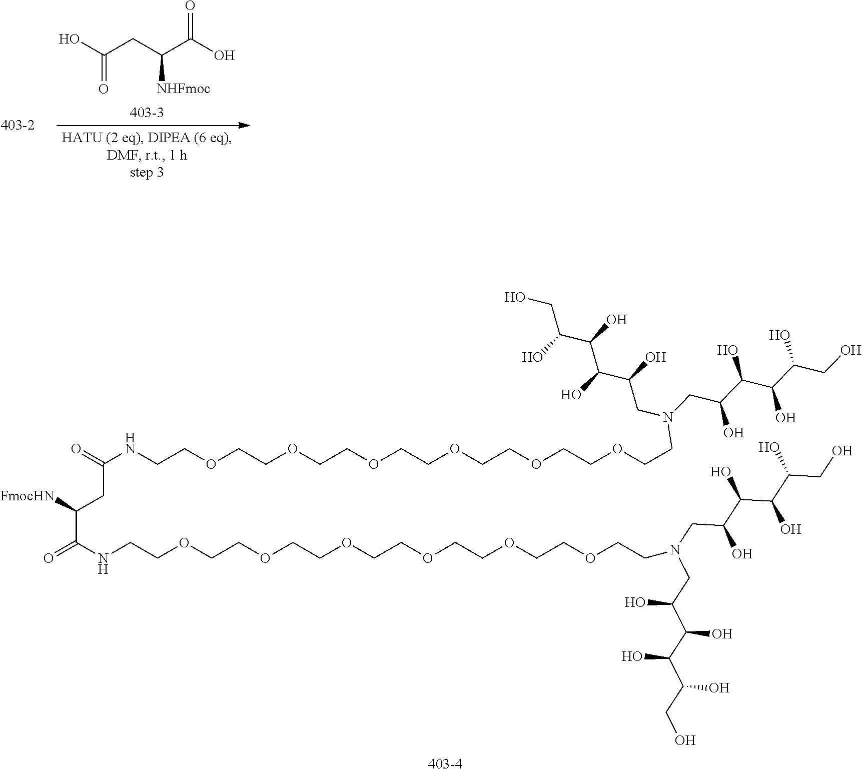

A solution of 403-2 (610 mg, 0.935 mmol), 403-3 (166 mg, 0.467 mmol) and HATU (355 mg, 0.934 mmol) in anhydrous DMF (5 mL) was stirred at room temperature for 5 min, then DIPEA (362 mg, 2.801 mmol) was added. The resulting solution was stirred for another 1 hr at r.t. until LCMS indicated complete reaction. The reaction solution was purified directly by reverse phase liquid chromatography to give 403-4 (410 mg, 0.252 mmol, 54.03%) as a white solid.

### Step 4

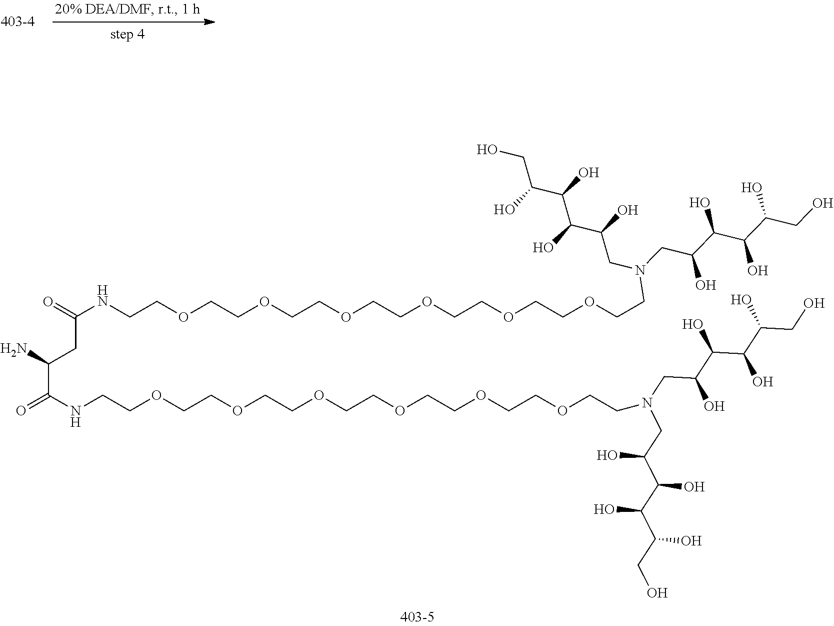 [SMILES: N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-4 (200 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 403-5 (167 mg, 0.119 mmol, 96.80%) as colorless oil, used as such in the next step.

### Step 5

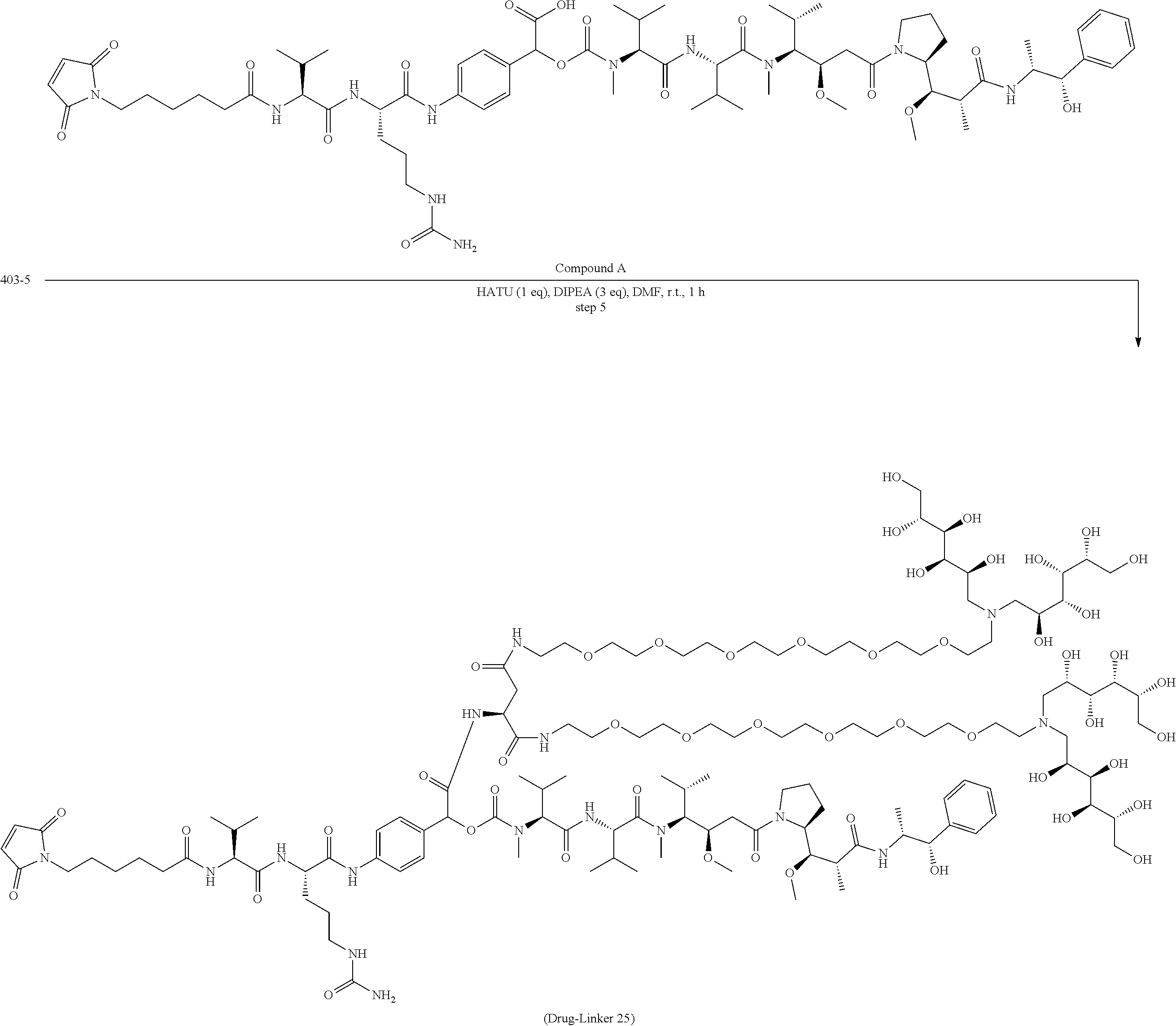

A solution of 403-5 (41 mg, 0.029 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 25 (18 mg, 0.007 mmol, 22.61%) as a white solid. LCMS, m/z=1373.31 (M/2+H)⁺.

### Example 26: Preparation of Drug-Linker 26

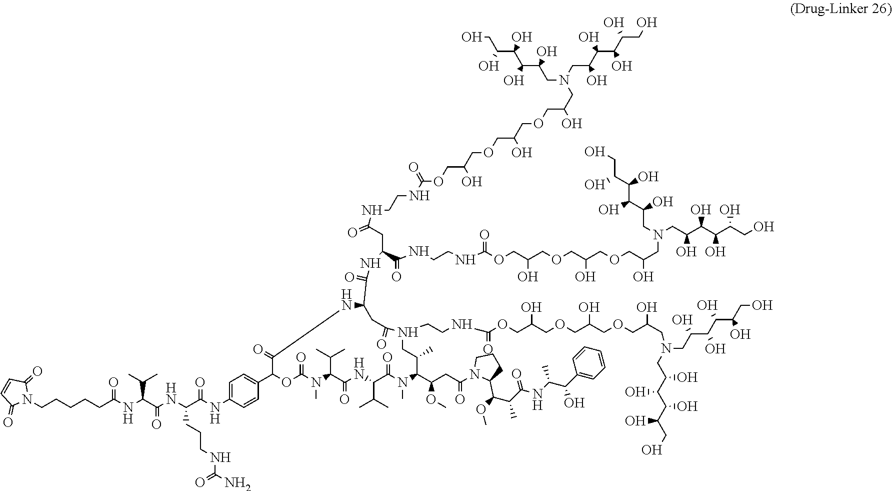

### Step 1

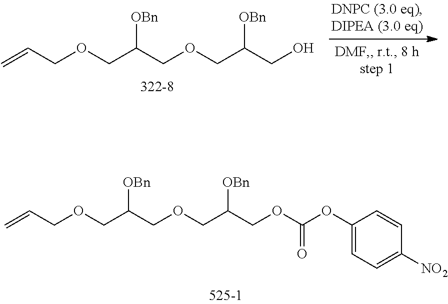 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 322-8 was consumed detected by LCMS. The reaction solution was directly used in the next step without work-up procedure.

### Step 2

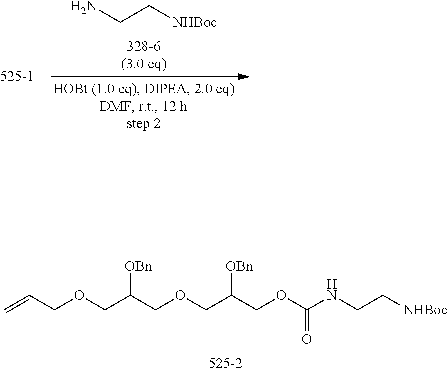 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

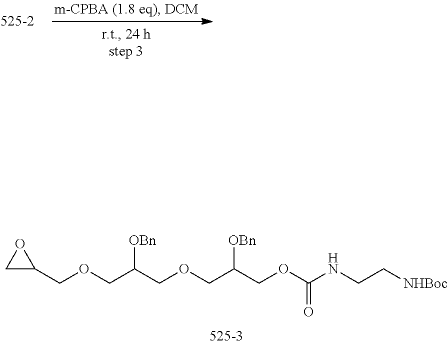 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃(aq., 10 mL). The reaction mixture was stirred for 30 mins, and then diluted with DCM (50 mL). The organic phase was washed with a mixture solution (20 mL×3) of saturated Na₂S₂O₃ and NaHCO₃(aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil. Purity=90%-95%.

### Step 4

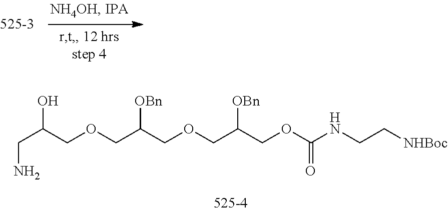 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

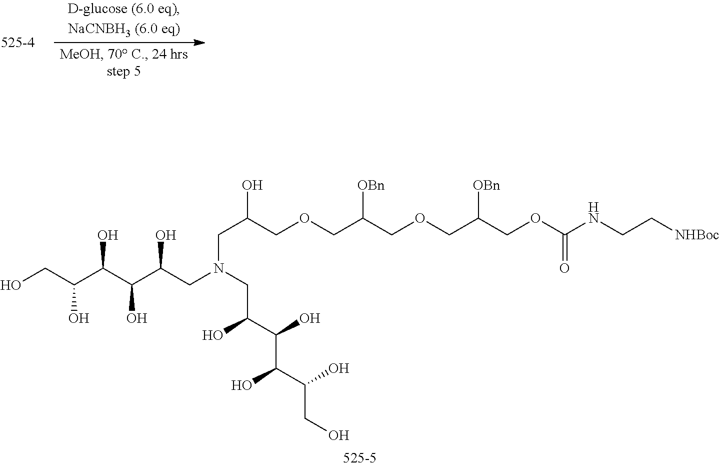 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

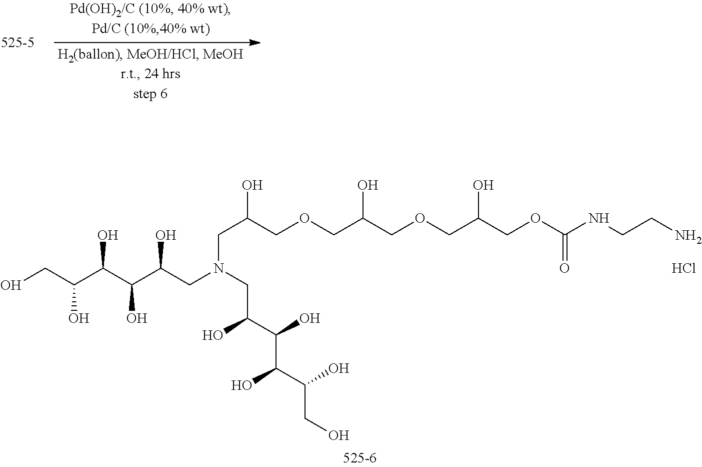 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (balloon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

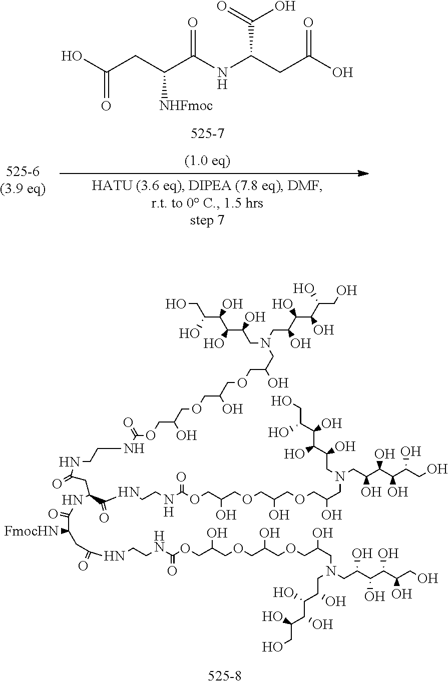

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 10H), 4.08-3.87 (m, 12H), 3.84-3.69 (m, 18H), 3.67-3.35 (m, 53H), 3.30-3.07 (m, 12H), 2.77-2.32 (m, 4H).

### Step 8

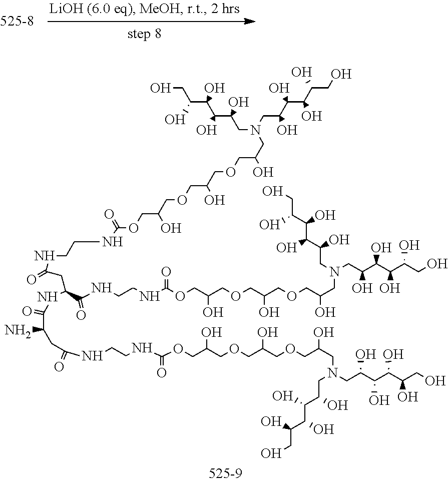 [SMILES: N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1 N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

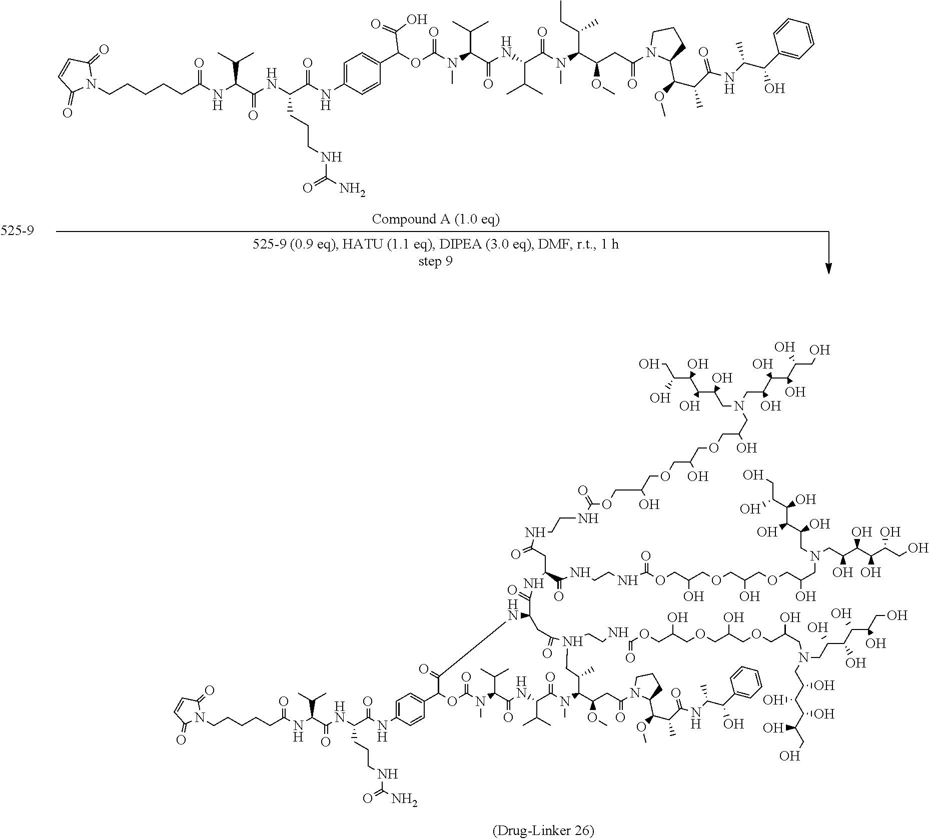

A solution of 525-9 (70.0 mg, 0.0325 mmol), Compound A(49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give drug-linker 26 (23.5 mg, 0.00672 mmol, 20.7%) as a white solid. LCMS, m/z=1749.66 (M/2+H)⁺, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 27: Preparation of Drug-Linker 27

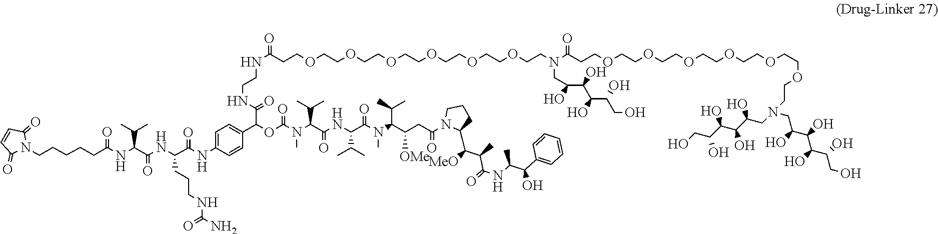

### Step 1

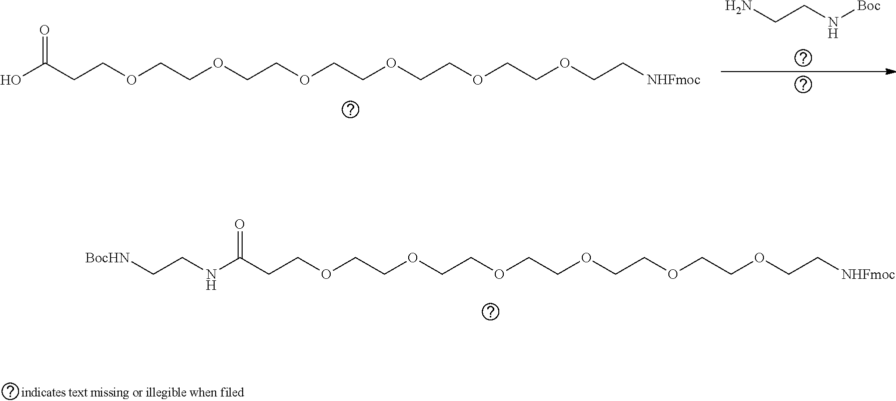

A solution of 341-1 (1 g, 1.737 mmol), 311-2 (278 mg, 1.735 mmol) and HATU (661 mg, 1.738 mmol) in anhydrous DMF (10 mL) was stirred at room temperature for 5 min, then DIPEA (674 mg, 5.215 mmol) was added. The resulting solution was stirred for another 1 hr at r.t. until LCMS indicated complete reaction. The reaction solution was purified directly by reverse phase liquid chromatography to give 341-2 (950 mg, 1.323 mmol, 76.17%) as a white solid. purity=90%-95%.

### Step 2

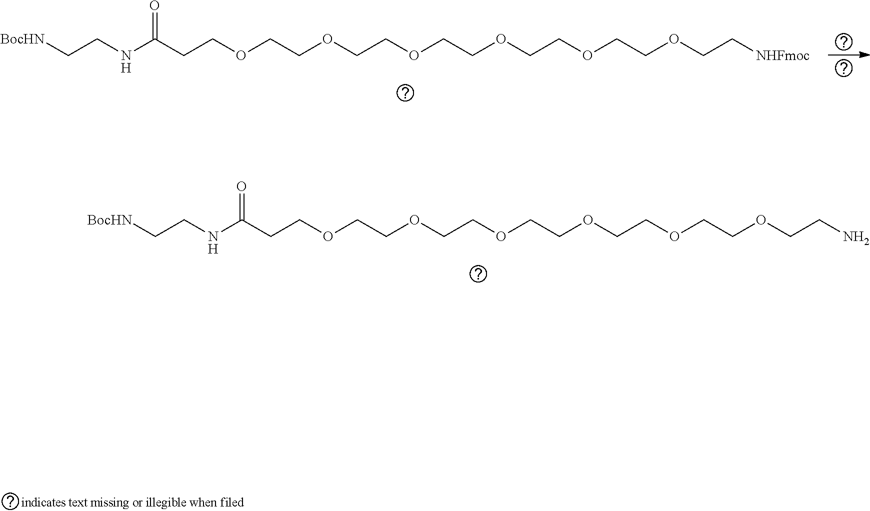

A solution of 341-2 (950 mg, 1.323 mmol) and DEA (2 mL) in anhydrous DMF (8 mL) was stirred at room temperature for 1 hr until LCMS indicated all starting amine was disappeared and desired product was detected. Then the solution was concentrated to dryness to 341-3 (610 mg, 1.231 mmol, 93.05%) as colorless oil, used as such in the next step. purity=90%-95%.

### Step 3

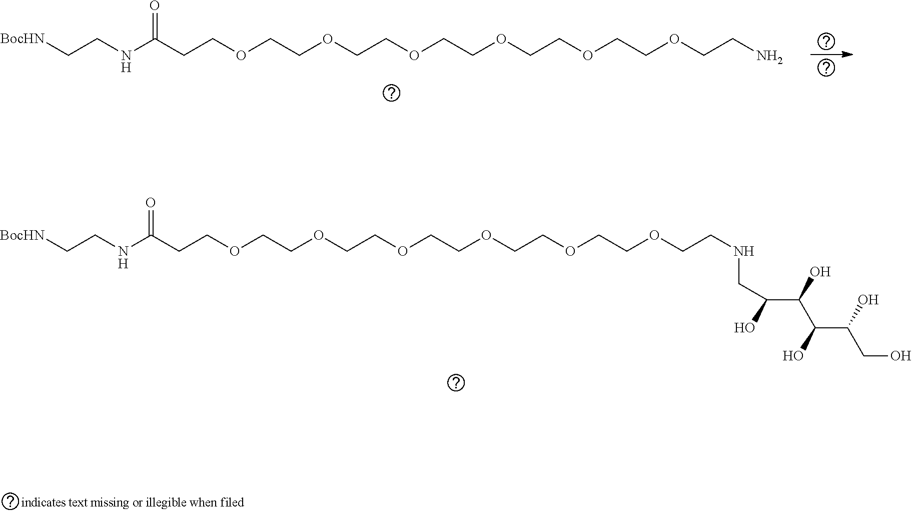 [SMILES: CC(C)(C)OC(=O)NCCNC(=O)CCOCCOCCOCCOCCOCCOCCN ; CC(C)(C)OC(=O)NCCNC(=O)CCOCCOCCOCCOCCOCCOCCNC[C@H](O)[C@@H](O)[C@H](O)[C@H](O)CO]

A solution of 341-3 (610 mg, 1.231 mmol) and D-Glucose (444 mg, 2.464 mmol) in anhydrous Methanol (50 mL) was heated at 50° C. for 30 min, then NaCNBH₃ (155 mg, 2.467 mmol) was added. The resulting solution was stirred for another 2 hr at 70° C. until indicated all starting amine was disappeared and the mass of desired product was detected. Then the reaction solution was concentrated and purified by reverse phase liquid chromatography to give 341-4 (340 mg, 0.515 mmol, 41.84%) as a white solid. purity=90%-95%.

### Step 4

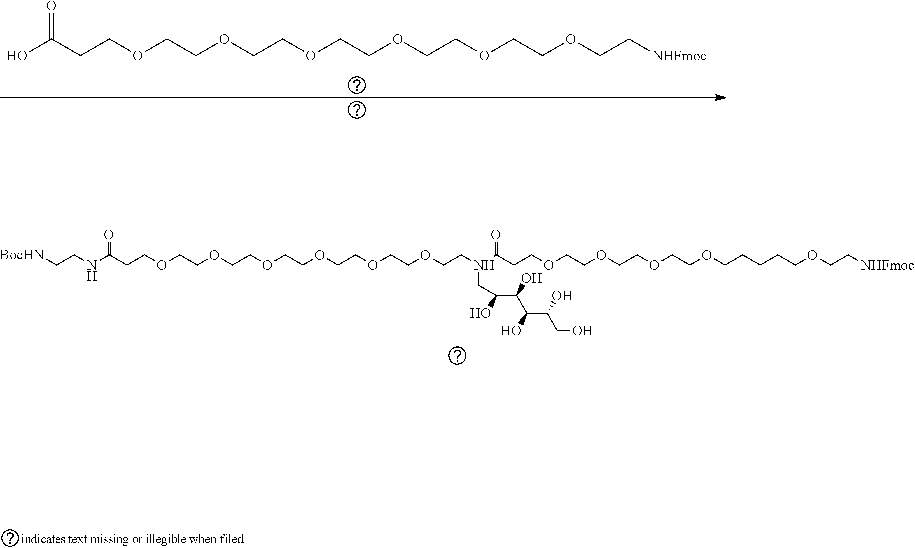

A solution of 341-4 (340 mg, 0.515 mmol), 341-1(297 mg, 0.516 mmol) and HATU (196 mg, 0.515 mmol) in anhydrous DMF (10 mL) was stirred at room temperature for 5 min, then DIPEA (200 mg, 1.548 mmol) was added. The resulting solution was stirred for another 1 hr at r.t. until LCMS indicated complete reaction. The reaction solution was purified directly by reverse phase liquid chromatography to give 341-5 (475 mg, 0.390 mmol, 75.73%) as a white solid. purity=90%-95%.

### Step 5

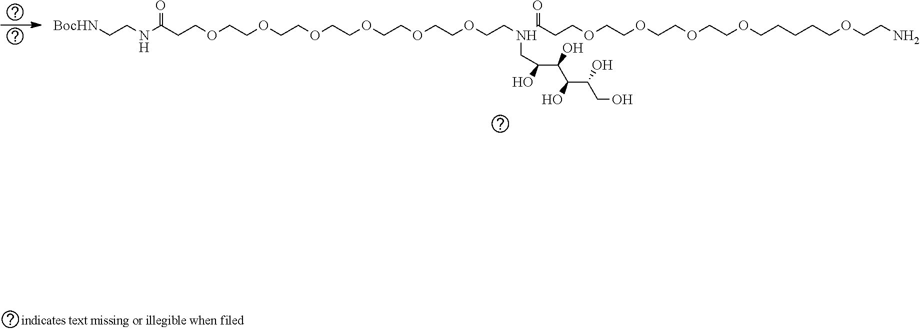 [SMILES: CC(C)(C)OC(=O)NCCNC(=O)CCOCCOCCOCCOCCOCCOCCN(C[C@H](O)[C@@H](O)[C@H](O)[C@H](O)CO)C(=O)CCOCCOCCOCCOCCCCCOCCN]

A solution of 341-5 (475 mg, 0.390 mmol) and DEA (2 mL) in anhydrous DMF (8 mL) was stirred at room temperature for 1 hr until LCMS indicated all starting amine was disappeared and desired product was detected. Then the solution was concentrated to dryness to 341-6 (372 mg, 0.374 mmol, 95.90%) as colorless oil, used as such in the next step. purity=90%-95%.

### Step 6

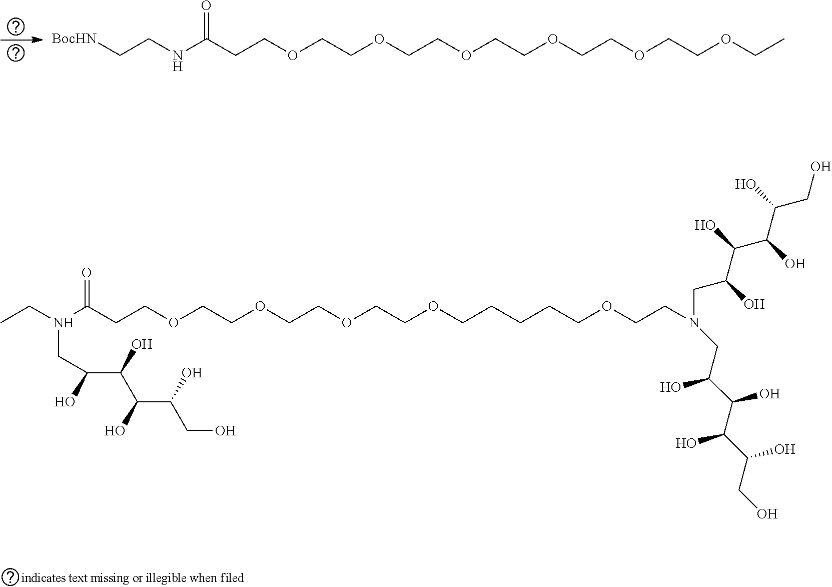

A solution of 341-6 (372 mg, 0.374 mmol) and D-Glucose (337 mg, 1.871 mmol) in anhydrous Methanol (50 mL) was heated at 50° C. for 30 min, then NaCNBH₃ (118 mg, 1.878 mmol) was added. The resulting solution was stirred for another 6 hr at 70° C. until indicated all starting amine was disappeared and the mass of desired product was detected. Then the reaction solution was concentrated and purified by reverse phase liquid chromatography to give 341-7 (406 mg, 0.307 mmol, 82.09%) as a white solid. purity=90%-95%.

### Step 7

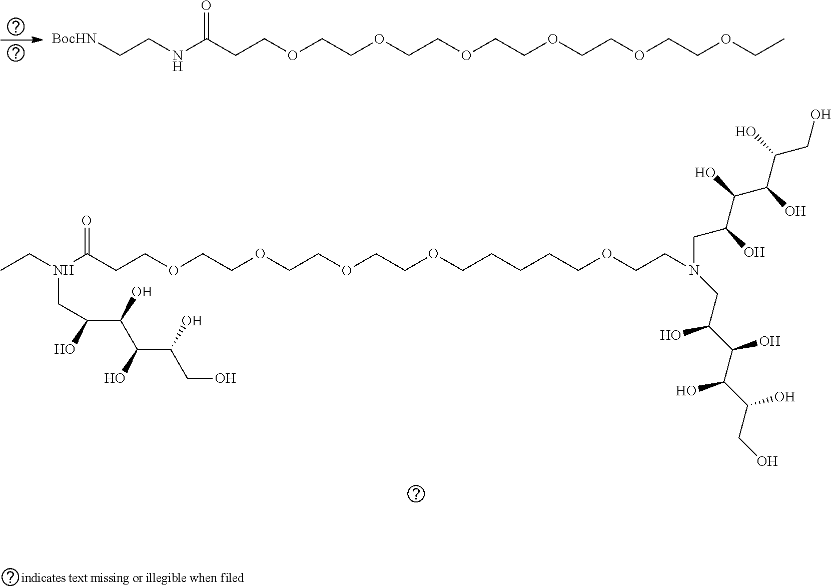

A solution of 341-7 (100 mg, 0.076 mmol) and TEA (2 mL) in anhydrous DCM (8 mL) was stirred at room temperature for 1 hr until LCMS indicated all starting amine was disappeared and desired product was detected. Then the solution was concentrated to dryness to 341-8 (85 mg, 0.069 mmol, 90.79%) as yellow oil, used as such in the next step. purity=90%-95%.

### Step 8

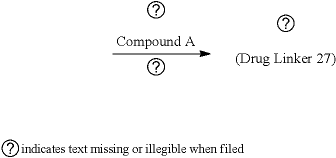

A solution of 341-8 (30 mg, 0.025 mmol), Compound A (33 mg, 0.025 mmol) and HATU (9 mg, 0.025 mmol) in anhydrous DMF (4 mL) was stirred at room temperature for 5 min, then DIPEA (10 mg, 0.074 mmol) was added. The resulting solution was stirred for another 1 hr at r.t. until LCMS indicated complete reaction. Then the reaction solution was purified by prep. HPLC to give drug-linker 27 (15 mg, 0.006 mmol, 24.01%) as a white solid. LCMS, m/z=856.38 (M/3+H)⁺.

### Example 28: Generation of Human Antibodies Against PTK7

The PTK7 extra-cellular domain (ECD) protein was prepared in house and used as antigen. Monoclonal antibodies against PTK7 were developed by sequentially immunizing mice with PTK7-his antigen proteins and an adjuvant, and the immunized animals included C₅₇bl/6 and Balb/c mice. The animals were immunized with 100 μg of antigen per animal for the first shot, subsequently 50 μg of antigen per animal was used for immunization for the second and third shots, and 25 μg for the booster immunization. The immune adjuvant used in the experiments was PAP-1. All animals were immunized by intraperitoneal injection. The mice with a good immunized titer were chosen for booster immunization.

After booster immunization, mice were sacrificed and soaked in 75% alcohol. The spleen was dissected out, ground with a grinding rod, and filtered through a cell strainer to prepare a single cell suspension. The spleen cell suspension was centrifuged at 1,500 rpm for 5 min, and the supernatant was discarded. 5 mL red blood cell lysate was added to lyse red blood cells at room temperature for 5 min and PBS was added to reach 20 mL. After centrifugation at 1,500 rpm for 5 min, the supernatant was discarded. Viable cells were counted after resuspension. Sp2/0 cells in a culture flask were collected and after centrifugation at 1,500 rpm for 5 min, the supernatant was discarded. Viable cells were counted after resuspension. The spleen cells were mixed with Sp2/0 cells at a ratio of 1.1:1 and subjected to centrifugation at 1,500 rpm for 5 min. The supernatant was discarded. The cells were resuspended in 20 mL electroporation buffer. After centrifugation at 1,500 rpm for 7 min, the supernatant was discarded and the step was repeated once.

The cells were resuspended with an appropriate amount of electroporation buffer to ensure the cell concentration of about 2×10⁷ cells/mL. The cell suspension was added to a 9 mL electroporation tank for fusion. After fusion, the cell suspension was transferred to 20 mL RPMI 1640 complete medium containing 20% FBS and then left at room temperature for 20 min. The fused cells were resuspended with RPMI 1640 medium containing 1×HAT, 1×BIOMYC3, and 20% FBS. The cell suspension was added to several 96-well cell culture plates at 100 ul/well to ensure that the cell volume per well was about 5×10⁴ cells/well, and the plates were placed in a 37° C. cell incubator. After 7 days, additional 100 μL of RPMI 1640 complete medium containing 20% FBS, 1×HAT, and 1×BIOMYC-3 was added to each well. After 10 days, the cell culture supernatants from hybridoma parent clones were collected and used for screening by binding to human and cynomolgus monkey PTK7-his protein by ELISA. The ELSIA positive clones were subsequently chosen for FACS binding test with MDA-MB-468 and the best three clones m2C5, m2C8 and m13C4 were chosen for humanization.

Murine antibodies generated (the clones m2C5, m2C8 and m13C4) were humanized using complementarity determining region (CDR) grafting. Human frameworks for heavy and light chains were selected based on sequence and structure similarity with respect to functional human germline genes. Structural similarity was evaluated by comparing the mouse canonical CDR structure to human candidates with the same canonical structures as described in Chothia et al. (supra). After homology comparison, IGKV1D-13+IGKJ2 and IGHV1-2+IGHJ4; IGKV4-1+IGKJ1 and IGHV1-2+IGHJ4; IGKV3-NL1+IGKJ2 and IGHV3-11+IGHJ3 were chosen for 2C5, 2C8, 13C₄ light chain and heavy chain framework region, respectively. Considering the antigenicity, activity and 3D structural similarity together, three sequences with back mutations were chosen as final molecules for humanized antibodies 2C5, 2C8 and 13C4. Sequences of the variable region and CDRs of anti-PTK7 antibodies including murine antibodies and humanized antibodies are shown in Tables 1 and 2.

TABLE 1
Variable region sequence of anti-PTK7 antibodies

| Antibody | VH | VL |
|---|---|---|
| m2C5 | EVQLLSSGPELVTPGASVKISCKASGYT | DILLTQSPAILSVSPGERVSFSCRASQ |
| | FTDYYINWLKQSHGKSLEWIGDINPNSG | NIGTSIHWYQQRTNGSPRLLIKFASESI |
| | GPVYNQKFMAKATLTVDKTSNTAYMEL | SGIPSRFSGSGSGTDFALTINSVESEDI |
| | RSLTSEDSAVYYCARGDYYGSNYNYW | ADYYCQQSNNWPYTFGGGTKLEIK |
| | GQGTTLTVSS (SEQ ID NO: 34) | (SEQ ID NO: 35) |
| m2C8 | QIQLVQSGPELKKPGETVKISCKASGYT | DVLMTQTPLSLPVSLGDQASISCRSS |
| | FTTYGMSWVKQAPGKGLKWMGWINTH | QSIVHNNGDTYLEWYLQKPGQSPKLLI |
| | SGVPTYVDEFKGRSAFSLETSASTAYLQ | YKVSNRFPGVPDRFSGSGSGTDFTLK |
| | INNLKNEDTATYFCARSPFDYGSRGAW | ISRVEAEDLGLYYCFQGSHVPWTFGG |
| | FVYWGQGTLVTVSS (SEQ ID NO: 48) | GTKLEIK (SEQ ID NO: 49) |
| m13C4 | EVKLVESGGGLVKPGGSLKLSCAASGF | DIVMTQSPAIMSTSPGEKVTMTCRASS |
| | AFSTYDMFWFRQTPEKRLEWVATISSG | SVSSSYLHWYQQKSGASPKLWIFRTS |
| | GGYTYYPGSVKGRFTISRDNARNTLYL | NLASGVPARFSGSGSGTSYSLTISSVE |
| | QMSSLRSEDTALYYCVRPLLRDSYFYF | AEDAATYYCQQYSGYPLTFGAGTKLE |
| | DVWGAGTTVTVSS (SEQ ID NO: 41) | LK (SEQ ID NO: 42) |
| 2C5 | QVQLVQSGAEVKKPGASVKVSCKASGY | EIVLTQSPATLSVSPGERATLSCRASQ |
| | TFTDYYINWVRQAPGQGLEWMGDINPY | NIGTSIHWYQQKPGQAPRLLIKFASESI |
| | SGGPVYNQKFMARVTMTVDKSINTAYM | SGIPARFSGSGSGTEFTLTISSLQSEDI |
| | ELSRLRSDDTAVYYCARGDYYGSNYNY | AVYYCQQSNNWPYTFGQGTKLEIK |
| | WGQGTLVTVSS (SEQ ID NO: 1) | (SEQ ID NO: 2) |
| 2C8 | QIQLVQSGAEVKKPGASVKVSCKASGY | DVVMTQSPDSLAVSLGERATINCRSS |
| | TFTTYGMSWVRQAPGQGLEWMGWINT | QSIVHNSGDTYLEWYQQKPGQPPKLL |
| | HSGVPTYVDEFKGRVTMTLDTSTSTAY | IYKVSNRFPGVPDRFSGSGSGTDFTLT |
| | MELSSLRSEDTAVYYCARSPFDYGSRG | ISSLQAEDLAVYYCFQGSHVPWTFGG |
| | AWFVYWGQGTTVTVSS (SEQ ID NO: | GTKVEIK (SEQ ID NO: 16) |
| | 15) | |
| 13C4 | QVQLVESGGGLVKPGGSLRLSCAASGF | EIVMTQSPATLSVSPGERATLSCRASS |
| | AFSTYDMFWIRQAPGKGLEWVSTISSG | SVSSSYLHWYQQKPGQAPRLLIFRTS |
| | GGYTYYPGSVKGRFTISRDNAKNSLYL | NLASGIPARFSGSGSGTEYTLTISSLQ |
| | QMNSLRAEDTAVYYCVRPLLRDSYFYF | SEDAAVYYCQQYSGYPLTFGQGTKLE |
| | DVWGQGTMVTVSS (SEQ ID NO: 8) | IK (SEQ ID NO: 9) |

TABLE 2
CDR sequences of anti-PTK7 antibodies

| Antibody | HCDR1 | HCDR2 | HCDR3 | LCDR1 | LCDR2 | LCDR3 |
|---|---|---|---|---|---|---|
| m2C5 | GYTFTDY | INPNSGG | ARGDYYGSNYNY | QNIGTS | FAS | QQSNNWP |
| | Y (SEQ ID | P (SEQ ID | (SEQ ID NO: 38) | (SEQ ID NO: | | YT (SEQ ID |
| | NO: 36) | NO: 37) | | 39) | | NO: 40) |
| m2C8 | GYTFTTY | INTHSGV | ARSPFDYGSRGAWF | QSIVHNNGD | KVS | FQGSHVP |
| | G (SEQ | P (SEQ ID | VY (SEQ ID | TY (SEQ ID | | WT (SEQ ID |
| | ID NO: | NO: 51) | NO: 52) | NO: 53) | | NO: 55) |
| | 50) | | | | | |
| m13C4 | GFAFSTY | ISSGGGY | VRPLLRDSYFYFDV | SSVSSSY | RTS | QQYSGYPL |
| | D (SEQ ID | T (SEQ ID | (SEQ ID NO: 45) | (SEQ ID NO: | | T (SEQ ID |
| | NO: 43) | NO: 44) | | 46) | | NO: 47) |
| 2C5 | GYTFTDY | INPYSGG | ARGDYYGSNYNY | QNIGTS | FAS | QQSNNWP |
| | Y (SEQ ID | P (SEQ ID | (SEQ ID NO: 5) | (SEQ ID NO: | | YT (SEQ ID |
| | NO: 3) | NO: 4) | | 6) | | NO: 7) |
| 2C8 | GYTFTTY | INTHSGV | ARSPFDYGSRGAWF | QSIVHNSGD | KVS | FQGSHVP |
| | G (SEQ | P (SEQ ID | VY (SEQ ID | TY (SEQ ID | | WT (SEQ ID |
| | ID NO: | NO: 18) | NO: 19) | NO: 20) | | NO: 22) |
| | 17) | | | | | |
| 13C4 | GFAFSTY | ISSGGGY | VRPLLRDSYFYFDV | SSVSSSY | RTS | QQYSGYPL |
| | D (SEQ ID | T (SEQ ID | (SEQ ID NO: 12) | (SEQ ID NO: | | T (SEQ ID |
| | NO: 10) | NO: 11) | | 13) | | NO: 14) |

Sequences of the CDR regions of the humanized antibody 2C8 using the IMGT and KABAT systems are shown in Table 3.

TABLE 3
CDRs sequences of humanized antibody 2C8 using the IMGT and KABAT systems

| System | HCDR1 | HCDR2 | HCDR3 | LCDR1 | LCDR2 | LCDR3 |
|---|---|---|---|---|---|---|
| IMGT | GYTFTT | INTHSGVP | ARSPFDYGSRG | QSIVHNSGDTY | KVS | FQGSHV |
| | YG (SEQ | (SEQ ID NO: | AWFVY (SEQ ID | (SEQ ID NO: | | PWT |
| | ID NO: | 24) | NO: 25) | 26) | | (SEQ ID |
| | 23) | | | | | NO: 27) |
| KABAT | TYGMS | WINTHSGVP | SPFDYGSRGAW | RSSQSIVHNSG | KVSN | FQGSHV |
| | (SEQ ID | TYVDEFKG | FVY (SEQ ID | DTYLE (SEQ ID | RFP | PWT |
| | NO: 28) | (SEQ ID NO: | NO: 30) | NO: 31) | (SEQ | (SEQ ID |
| | | 29) | | | ID NO: | NO: 33) |
| | | | | | 32) | |

### Example 29: Binding Activity of Humanized Antibodies to PTK7 Protein

Binding specificity property was tested by ELISA according to a standard protocol. Specifically, 96-well micro-plates were coated with 2 μg/mL human, cynomolgus, mouse, and rat PTK-7 recombinant protein in PBS 100 μL per well, incubated overnight at 4° C. Plates were washed twice by TBS+0.5% Tween20. 200 μL of blocking buffer (2% BSA in PBS) was added to each well and incubated at 37° C. for 2 hours. Plates were washed using the buffer mentioned above. Serially diluted antibodies were added to the ELISA plate with 100 μL per well and incubated for 1 hour at room temperature. Then plates were washed for 3 times. After washing, HRP conjugated anti-human Fc antibody solution (abcam, ab98624, diluted with blocking buffer) was added to the plate with 100 μl per well. The plates were incubated at room temperature for 1 hour and then washed for 3 times. Then TMB solution was added to plates with 100 μl per well, placed at room temperature for 5-15 mins, then the stop solution (2M H₂SO₄) was added with 50 μl per well. Finally, the result was measured by absorbance at A₄₅₀ and A₆₃₀.

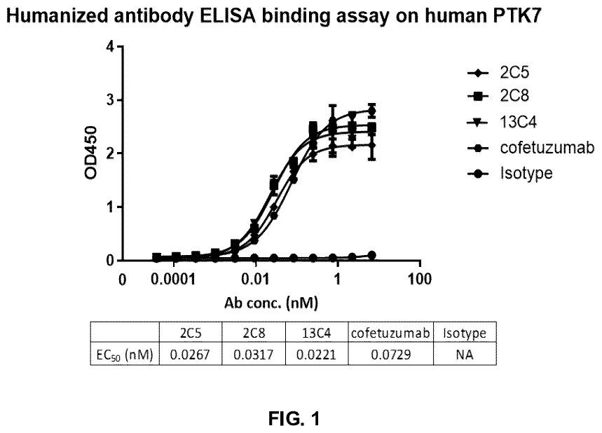

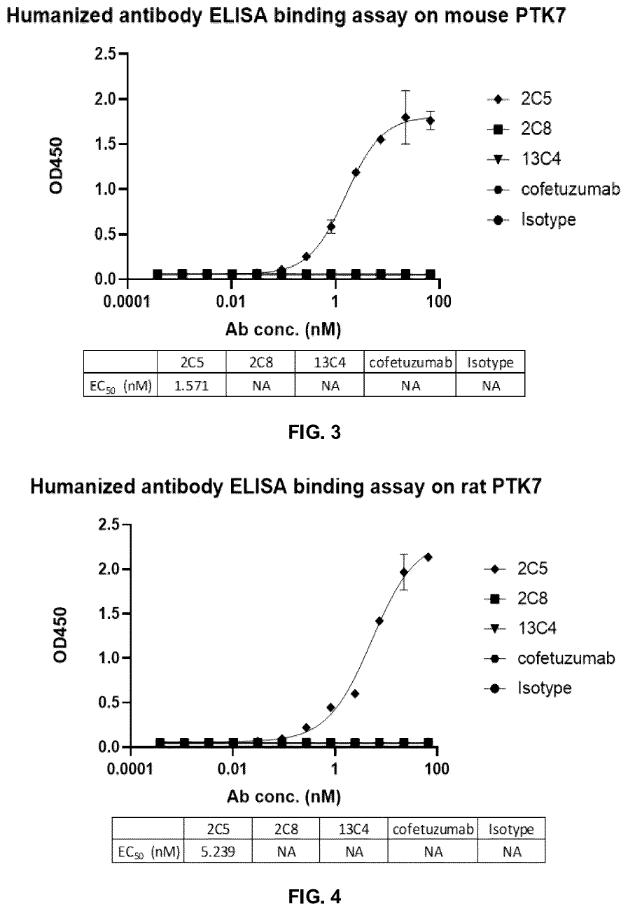

ELISA binding assay results of humanized antibodies 2C5, 2C8, 13C4 and cofetuzumab (as a comparator) are illustrated in FIGS. 1 to 4. As shown in FIGS. 1 to 4, 2C5, 2C8 and 13C4 showed similar binding activity to human PTK7 protein (EC₅₀=0.027, 0.032 and 0.022 nM, respectively) as compared to cofetuzumab (EC₅₀=0.073 nM). 2C5, 2C8 and 13C4 showed similar binding activity to cynomolgus monkey PTK7 protein (EC₅₀=1.665, 2.606 and 1.135 nM, respectively) as compared to cofetuzumab (EC₅₀=1.042 nM). 2C5 showed strong binding activity to rat and mouse PTK7 protein with the EC₅₀ of 1.57 and 5.24 nM, respectively. 2C8 and 13C4 displayed no cross-reactivity to rat or mouse PTK7.

### Example 30: Affinity of Humanized Antibodies to PTK7

Recombinant proteins consisting of the PTK7 extra-cellular domain (ECD) linked to His tag were purchased from ACRO biosystems. 2C₈ or PRO1107 (13 nM) was immobilized on anti-human IgG Fc biosensor tips (ForteBio). Binding assays using varying concentrations from 100 nM down to 1.56 nM of recombinant proteins (human, cynomolgus) in solution were performed using Octet RED (ForteBio). Association time was set at 180 seconds and dissociation time was set at 300 seconds. Binding affinity was calculated using ForteBio Data Acquisition 6.3 software (ForteBio). Affinity was derived by fitting the kinetic data to a 1:1 Langmuir binding model utilizing global fitting algorithms. The results are shown in Table 4.

TABLE 4
Affinity results of humanized antibodies to
human and cynomolgus monkey PTK7

| Immobilized | | KD | ka | kdis |
|---|---|---|---|---|
| Molecule | mAb | (M) | (1/Ms) | (1/s) |
| hPTK7-his | 2C5 | 1.39E−12 | 3.48E+04 | <1.0E−07 |
| | 2C8 | 5.22E−09 | 2.51E+05 | 9.93E−05 |
| | 13C4 | 7.98E−10 | 1.66E+05 | 1.32E−04 |
| | cofetuzumab | <1.0E−12 | 5.02E+04 | <1.0E−07 |
| cPTK7-his | 2C5 | 9.04E−11 | 2.24E+05 | 2.02E−05 |
| | 2C8 | 1.99E−08 | 5.33E+05 | 2.78E−03 |
| | 13C4 | <1.0E−12 | 5.41E+04 | <1.0E−07 |
| | cofetuzumab | 1.04E−12 | 4.65E+04 | <1.0E−07 |

2C5, 2C8 and 13C4 showed high affinity to human PTK7 with equilibrium dissociation constant (KD) of 1.39×10⁻¹² M, 5.22×10⁻⁹ M, and 7.98×10⁻¹⁰ M, respectively. 2C5, 2C8 and 13C4 showed high affinity to cynomolgus monkey PTK7 with equilibrium dissociation constant (KD) of 9.04×10⁻¹¹ M, 1.99×10⁻⁸ M, and 10⁻¹² M, respectively.

### Example 31: Binding Activity of Humanized Antibodies to Tumor Cells

Antibodies were tested for binding to renal cells and glioblastoma cells expressing PTK7 on the cell surface by flow cytometry. The cell lines PA-1 (ATCC® CRL-1572™, Provided by Procell), MDA-MB-468 (ATCC® HTB-132™, Provided by Procell), MDA-MB-231 (ATCC® CRM-HTB-26™, Provided by COBIOER), and MDA-MB-453 (ATCC® HTB-131™, Provided by COBIOER) were each tested for antibody binding. PA-1 was cultured with MEM medium (Gibco, Cat #11095-080) containing of 10% FBS (Cellmax, Cat #SA211.02) while MDA-MB-468 and MDA-MB-231 were cultured with RPMI 1640 (Gibco, Cat #11875093) containing 10% FBS, MDA-MB-453 was cultured with DMEM medium (Gibco, Cat #C11995500BT) containing of 10% FBS. Each antibody was incubated for 30 mins with different cell lines (3×10⁵ cells per well) in 0.2 mL FACS buffer (1×PBS with 0.1% BSA) at 4° C. Then, the cells were pelleted, washed, and incubated at 4° C. for 30 mins with 100 μL of 1:200 diluted PE-conjugated anti human Fc (Abcam, Ab98596) in FACS buffer. The cells were pelleted again, washed with PBS, resuspended in FACS buffer and analyzed by flow cytometer (Beckman, CytoFLEX).

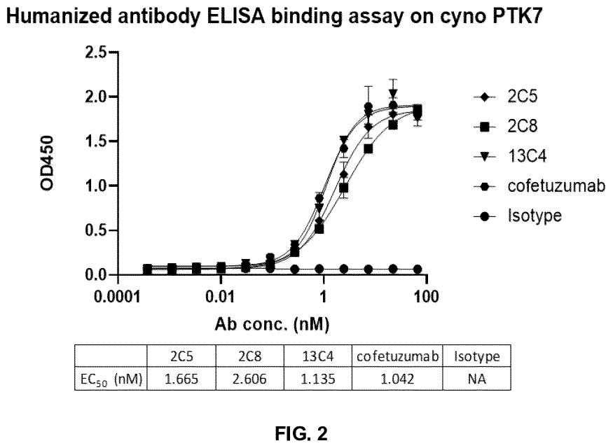

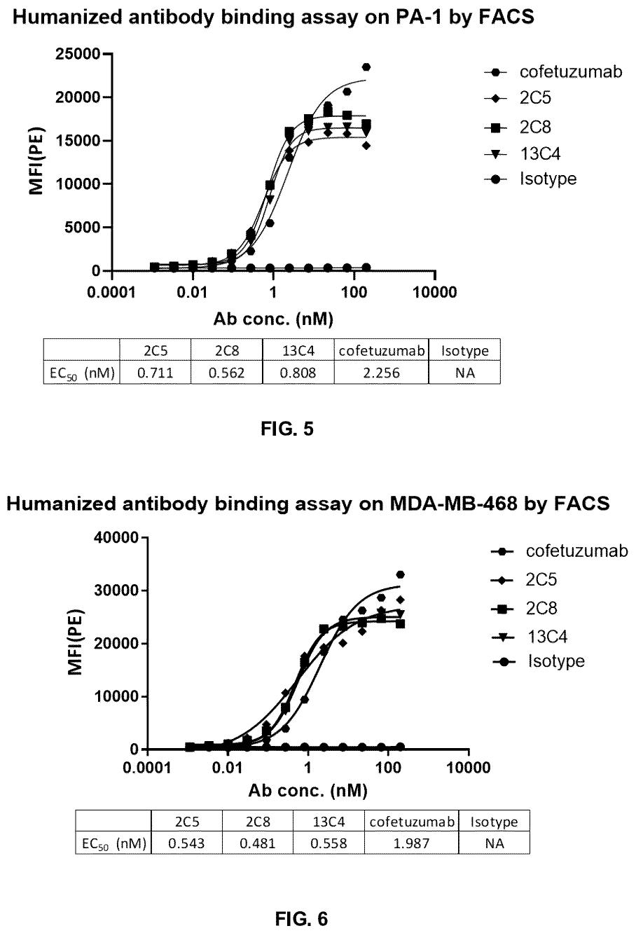

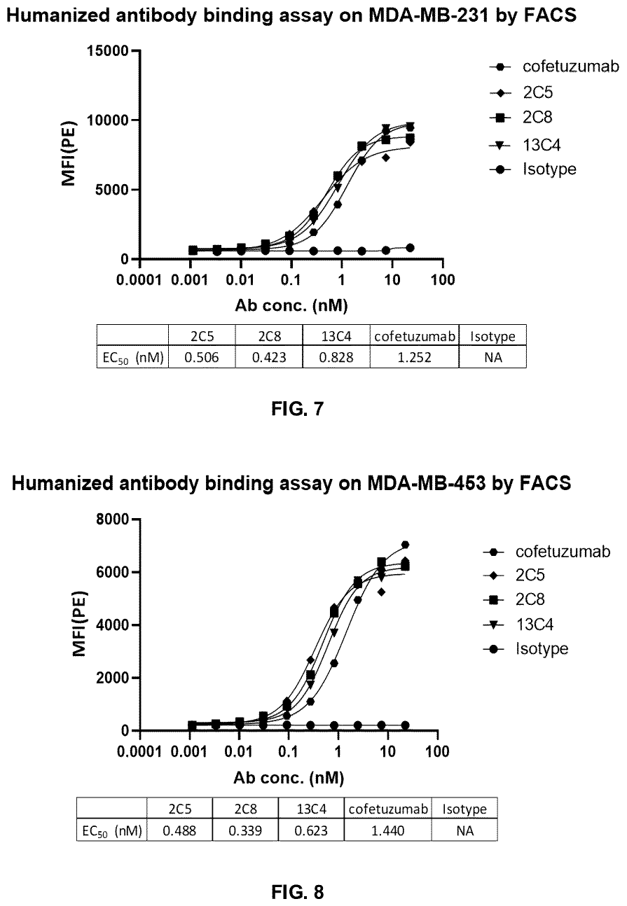

FIGS. 5-8 illustrate the binding activity of antibodies to tumor cells respectively. As shown in FIGS. 5-8, 2C5, 2C8 and 13C4 showed stronger binding activity than cofetuzumab with the EC₅₀ in the range of 0.34-0.81 nM on human PTK7-expressing tumor cell lines while cofetuzumab showed binding activity with the EC₅₀ in the range of 1.25-2.26 nM. All of 2C5, 2C8 and 13C4 have stronger tumor cell binding than cofetuzumab.

### Example 32: Internalization of Humanized Antibodies

The internalization assay was conducted in time course. 3×10⁵ Cells were incubated for 30 min at 4° C. with 10 μg/mL of 2C5, 2C8, 13C4, or cofetuzumab in FACS buffer (1×PBS containing 0.1% BSA). Cells were washed at 4° C. to remove unbound material and kept on ice or shifted to 37° C. as needed. At progressive time points (0, 0.5, 2, 4 h), cells were stained with PE-conjugated anti-human Fc for 30 min at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0.

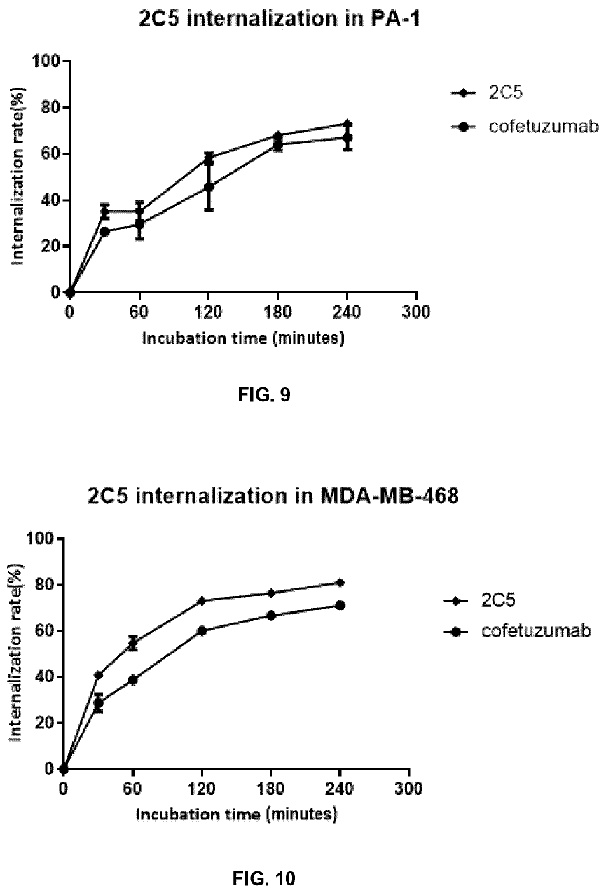

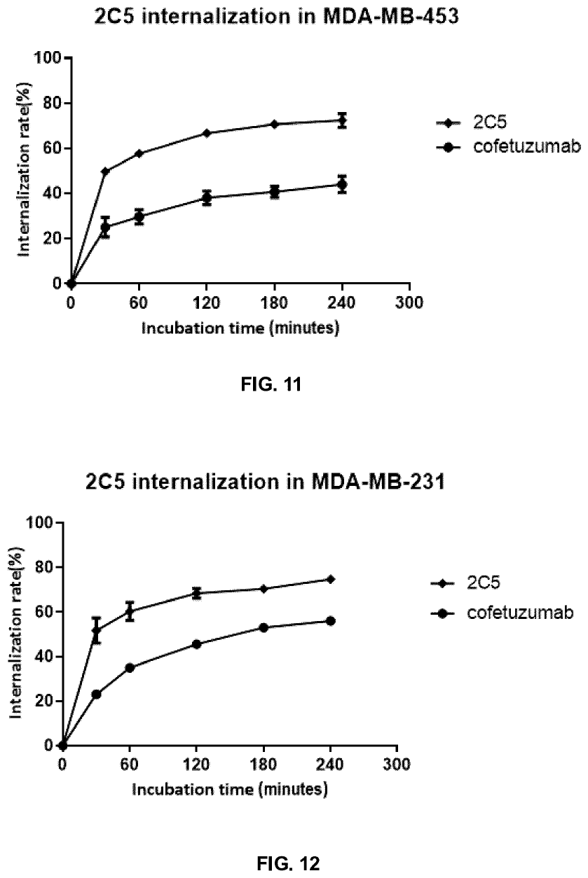

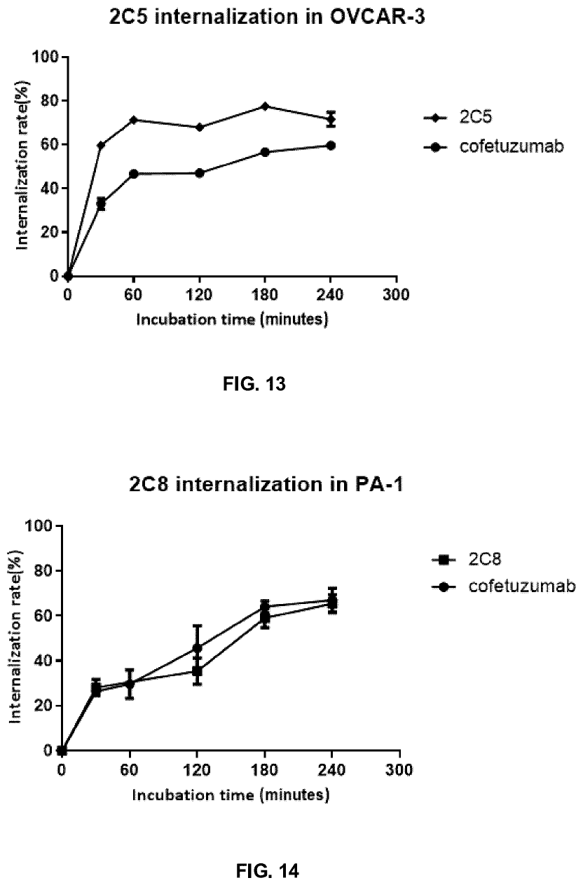

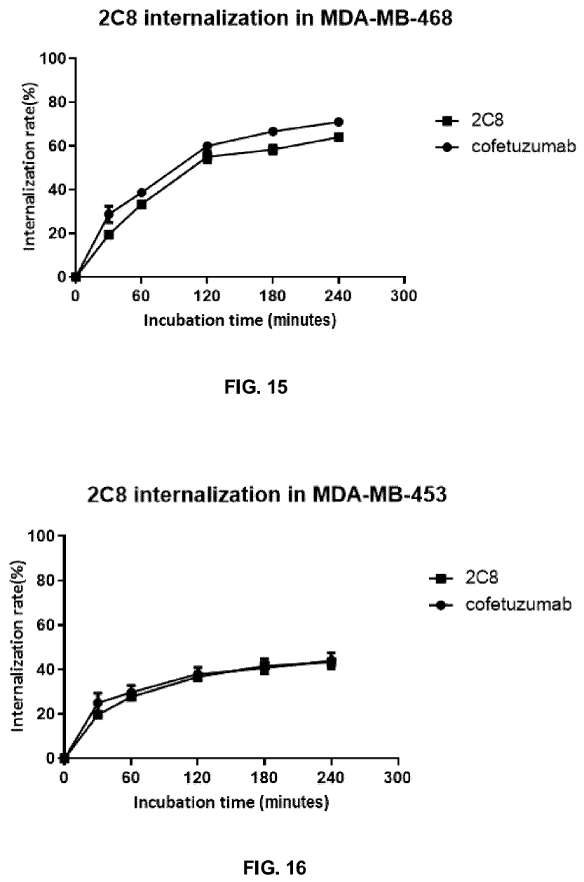

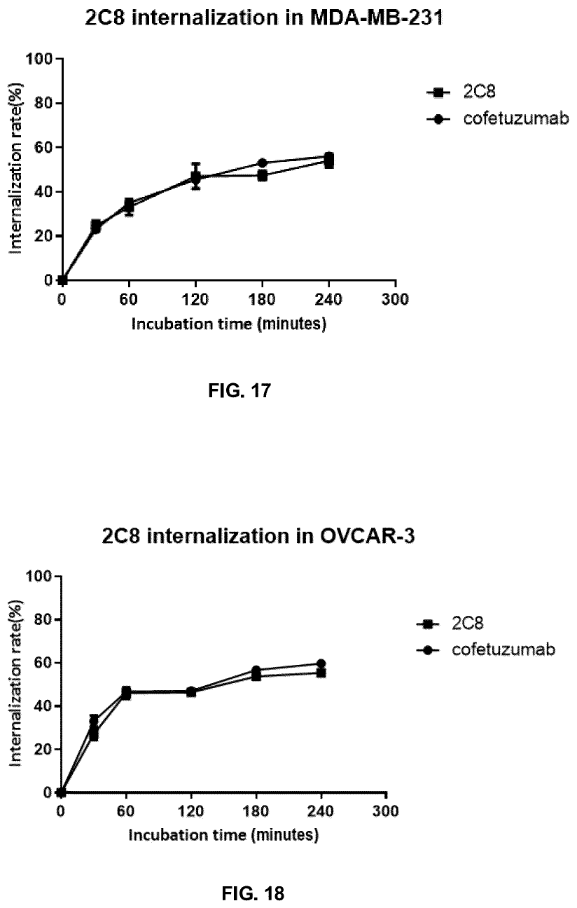

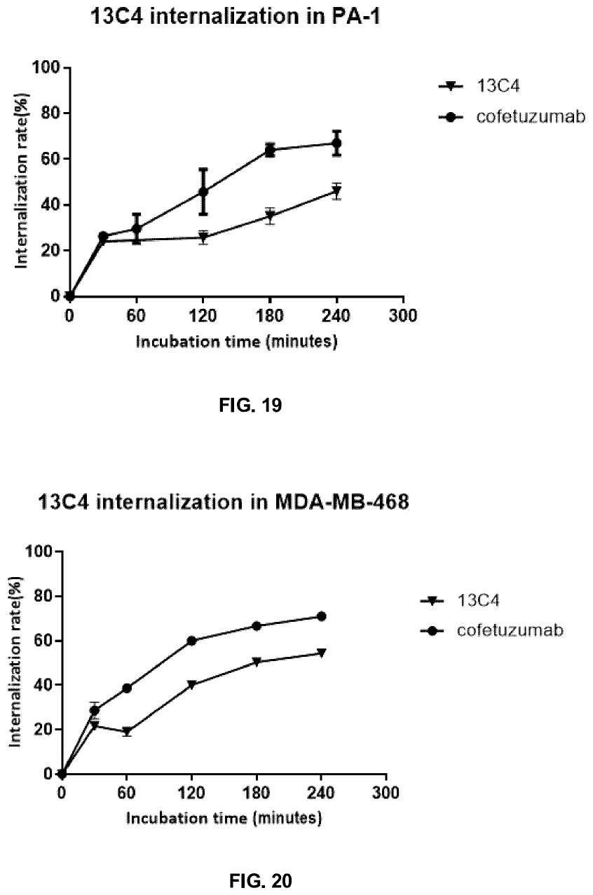

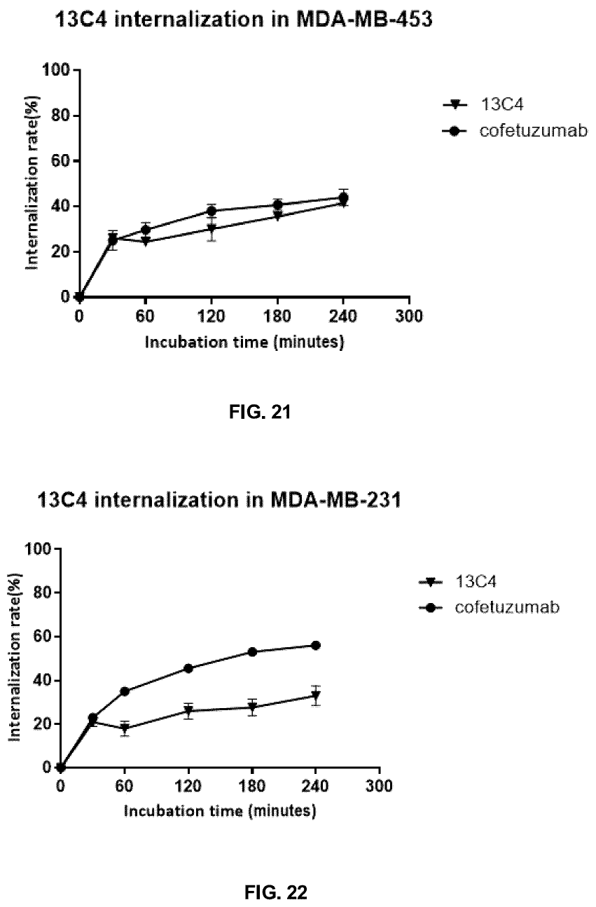

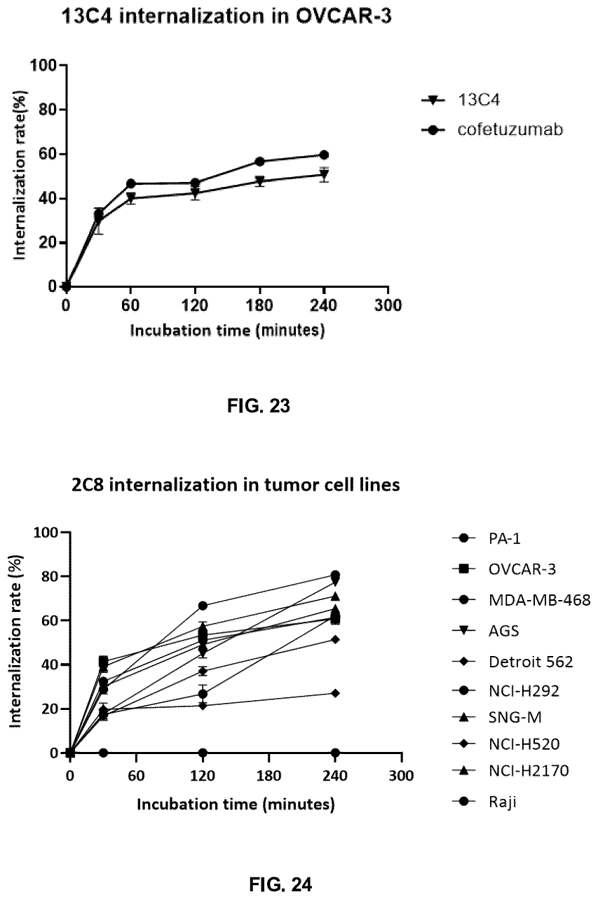

FIGS. 9-13 illustrate internalization rates of 2C5 and cofetuzumab in cell lines PA-1, MDA-MB-468, MDA-MB-453, MDA-MB-231, and OVCAR-3, respectively. FIGS. 14-18 illustrate internalization rates of 2C8 and cofetuzumab in cell lines PA-1, MDA-MB-468, MDA-MB-453, MDA-MB-231, and OVCAR-3, respectively. FIGS. 19-23 illustrate internalization rates of 13C4 and cofetuzumab in cell lines PA-1, MDA-MB-468, MDA-MB-453, MDA-MB-231, and OVCAR-3, respectively. As shown in FIGS. 19-23, 2C5 displayed rapid and better internalization than cofetuzumab, 2C8 and 13C4 displayed similar internalization to cofetuzumab in multiple PTK7-expressing cell lines, i.e., PA-1, MDA-MB-468, MDA-MB-453, MDA-MB-231, and OVCAR-3.

### Example 33: Preparation of ADCs

Antibodies 2C5, 2C8, 13C4, and cofetuzumab were linked with drug-linker LD110 to obtain ADCs 2C5-LD110 (8), 2C8-LD110 (8), 13C4-LD110 (8), and cofetuzumab-LD110 (8). Specifically, 2 mL of each antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 8.0. Reducing reaction was conducted for 2 hours at 25° C. The excess TCEP and its byproduct was removed by ultrafiltration with pH=6.9 50 mM sodium phosphate buffer. LD110 (salt of TFA) was dissolved in water at a concentration of 20 mg/mL and added to reduced mAb at a molar ratio of 8.5 (LD110/mAb). The coupling reaction was stirred for 2 hours at 25° C. The excess LD110 and its impurities were removed by ultrafiltration with 50 mM sodium phosphate buffer. The ADC was stored in 20 mM histidine buffer containing 6% sucrose and 0.02% (w/V) Tween 20 by UFDF. The purity of each ADC as determined by SEC-HPLC was 98.0% and DAR value as determined by LC-MS was 7.5.

Antibodies 2C5, 2C8, 13C4, and cofetuzumab were linked with drug-linker LD038 to obtain ADCs 2C5-LD038 (8), 2C8-LD038 (8), 13C4-LD038 (8), and cofetuzumab-LD038 (8). Specifically, 2 mL of each antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 10.0. The reducing reaction was conducted for 2 hours at 25° C. The excess TCEP and its byproduct were removed by ultrafiltration with pH=6.9 50 mM sodium phosphate buffer. LD038 (salt of TFA) was dissolved in water at a concentration of 20 mg/mL and added to reduced mAb at a molar ratio of 9.5 (LD038/mAb). The coupling reaction was stirred for 2 hours at 25° C. The excess LD038 and its impurities were removed by ultrafiltration with 50 mM sodium phosphate buffer. The ADC was stored in 20 mM histidine buffer containing 6% sucrose and 0.02% (w/V) Tween 20 by UFDF. The purity of each ADC as determined by SEC-HPLC was 98.0% and DAR value as determined by LC-MS was 8.0.

Antibody 2C8 was linked with drug-linker LD163 to obtain ADC 2C8-LD163 (8). Specifically, 2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 8.0. The reducing reaction was conducted for 2 hours at 25° C. The excess TCEP and its byproduct were removed by ultrafiltration with pH=6.9 50 mM sodium phosphate buffer. LD163 (salt of TFA) was dissolved in water at a concentration of 20 mg/mL and added to reduced mAb at a molar ratio of 8.5 (LD163/mAb). The coupling reaction was stirred for 2 hours at 25° C. The excess LD163 and its impurities were removed by ultrafiltration with 50 mM sodium phosphate buffer. The ADC was stored in 20 mM histidine buffer containing 6% sucrose and 0.02% (w/V) Tween 20 by UFDF. The purity of ADC as determined by SEC-HPLC was 98.0% and DAR value as determined by LC-MS was 7.5.

Antibody 2C8 was linked with drug-linker LD343 to obtain ADCs 2C8-LD343 (8) (also referred to as PRO1107). Specifically, 2 mL of each antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 8.0. The reducing reaction was conducted for 2 hours at 25° C. The excess TCEP and its byproduct were removed by ultrafiltration with pH=6.9 50 mM sodium phosphate buffer. LD343 (salt of TFA) was dissolved in water at a concentration of 20 mg/mL and added to reduced mAb at a molar ratio of 8.5 (LD343/mAb). The coupling reaction was stirred for 2 hours at 25° C. The excess LD343 and its impurities were removed by ultrafiltration with 50 mM sodium phosphate buffer. The ADC was stored in 20 mM histidine buffer containing 6% sucrose and 0.02% (wN) Tween 20 by UFDF. The purity of each ADC as determined by SEC-HPLC was 98.0% and DAR value as determined by LC-MS was 7.5.

Antibody cofetuzumab was linked with drug-linker pelidotin (an auristatin microtubule inhibitor payload attached to a cleavable linker) to obtain ADC cofetuzumab pelidotin. Specifically, 2 mL of antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 2.2. The reducing reaction was conducted for 2 hours at 25° C. Pelidotin was dissolved in DMSO at a concentration of 20 mg/mL and added to reduced mAb at a molar ratio of 5.0 (pelidotin/mAb). The coupling reaction was stirred for 2 hours at 25° C. The excess pelidotin and its impurities were removed by ultrafiltration with 50 mM sodium phosphate buffer. The ADC was stored in 20 mM histidine buffer containing 6% sucrose and 0.02% (wV) Tween 20 by UFDF. The purity of ADC as determined by SEC-HPLC was 97.0% and DAR value as determined by LC-MS was 3.7.

Antibody 2C₈ was linked with vedotin (mc-vcMMAE) to obtain ADCs 2C₈-vedotin (4). Specifically, 2 mL of each antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb was 2.2. The reducing reaction was conducted for 2 hours at 25° C. Mc-vcMMAE was dissolved in DMSO at a concentration of 20 mg/mL and added to reduced mAb at a molar ratio of 5.0 (Mc-vcMMAE/mAb). The coupling reaction was stirred for 2 hours at 25° C. The excess Mc-vcMMAE and its impurities were removed by ultrafiltration with 50 mM sodium phosphate buffer. The ADO was stored in 20 mM histidine buffer containing 6% sucrose and 0.02% (w/V) Tween 20 by UFDF. The purity of each ADO as determined by SEC-HPLC was 97.0% and OAR value as determined by LC-MS was 3.7.

TABLE 5
Linker-drug abbreviation and
corresponding linker, drug and DAR

| Linker-payload | | | |
|---|---|---|---|
| abbreviation | Linker | Drug | DAR |
| LD110 (8) | Proprietary | MMAE | 8 |
| LD163 (8) | Proprietary | MMAE | 8 |
| LD343 (8) | Proprietary | MMAE | 8 |
| vedotin (4) | mc-vc-PAB | MMAE | 4 |
| LD038 (8) | Proprietary | exatecan | 8 |

### Example 34: Similar In Vitro Internalization of 2C8 and PRO1107 in Various Tumor Cell Lines

The internalization assay was conducted in time course. 3×10⁵ Cells were incubated for 30 min at 4° C. with 10 μg/mL of 208 or PRO1107 in FACS buffer (1×PBS containing 0.1% BSA). Cells were washed at 4° C. to remove unbound material and kept on ice or shifted to 37° C. as needed. At progressive time points (0, 0.5, 2, 4 h), cells were stained with PE-conjugated anti-human Fc for 30 min at 4° C. and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time 0, then divided by the MFI of cell surface-bound antibody at 4° C. at time 0.

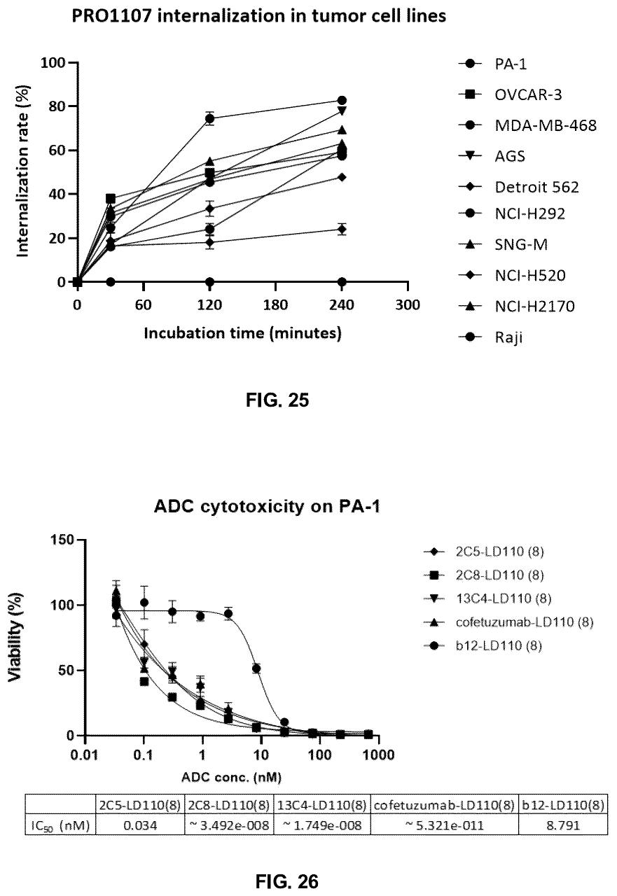

FIGS. 24 and 25 illustrate internalization rates of 208 and PRO1107 in various tumor cell lines, respectively. As shown in FIGS. 24 and 25, 2C8 and PRO1107 displayed rapid internalization on multiple PTK7-expressing cell lines (PA-1, OVCAR3, MDA-MB-468, AGS, Detroit 562, NCI-H292, SNG-M, NCI-H520, NCI-H2170) while no internalization was observed on PTK7 non-expressing cells (Raji). In addition, 2C8 and PRO1107 have similar in vitro internalization in these tumor cell lines.

### Example 35: Cytotoxicity Comparison of ADCs with Similar Linker-Drug LD110

One day prior to adding test article (2C5-LD110 (8), 2C8-LD110 (8), 13C4-LD110 (8), cofetuzumab-LD110 (8) or b12-LD110 (8) (used as a negative control), cells were harvested and plated into 96-well solid white flat bottom plates. The next day cells were exposed to the test article at concentrations from 666.67 to 0.034 nM. Plates were incubated at 37° C. for 96 h. After that, 40 μL Cell-titre Glo (CTG) per well was added to the plates with luciferase readings collected at 5 min after and analyzed by Microplate readers. All readings were normalized as percentage of viable cells in the untreated control wells and the IC₅₀ values were calculated by Prism software.

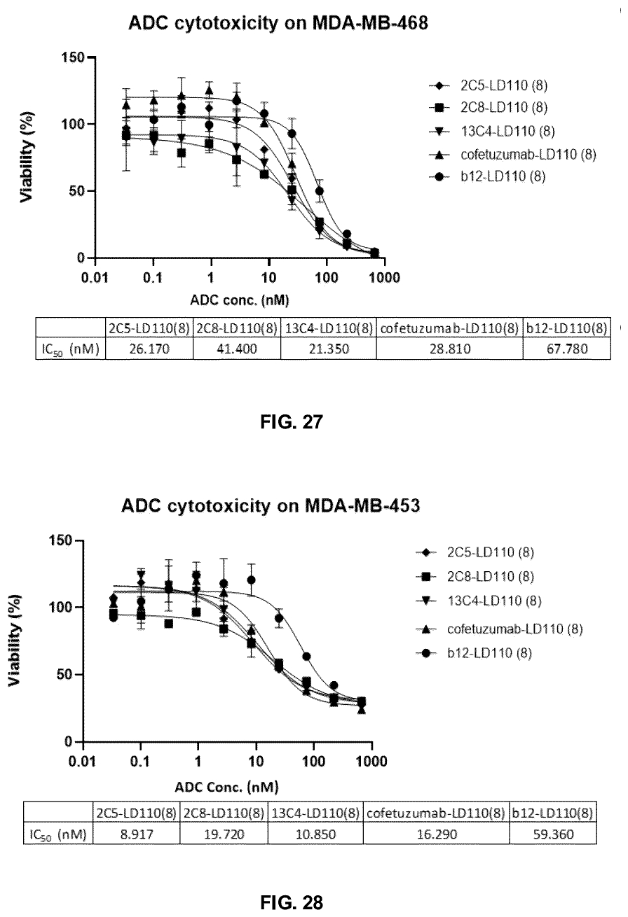

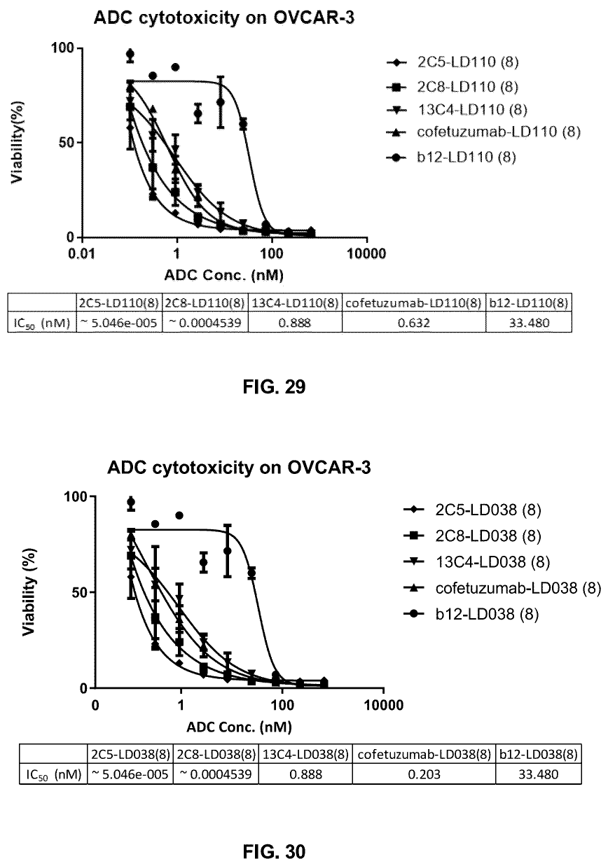

FIGS. 26-29 illustrate cytotoxicity of ADCs with linker-drug LD110 on tumor cell lines PA-1, MDA-MB-468, MDA-MB-453, and OVCAR-3, respectively. As shown in FIGS. 26-29, all candidates (2C5, 2C8, and 13C4) have better in vitro cytotoxicity than cofetuzumab when conjugated with similar linker-payload, LD110 on human PTK7-expressing tumor cell lines.

### Example 36: Cytotoxicity Comparison of ADCs with Similar Linker-Drug LD038

One day prior to adding test article (2C5-LD038 (8), 2C8-LD038 (8), 13C4-LD038 (8), cofetuzumab-LD038 (8) or b12-LD038 (8) (used as a negative control), cells were harvested and plated into 96-well solid white flat bottom plates. The next day cells were exposed to the test article at concentrations from 666.67 to 0.034 nM. Plates were incubated at 37° C. for 144 h. After that, 40 μl Cell-titre Glo (CTG) per well was added to the plates with luciferase readings collected at 5 min after and analyzed by Microplate readers. All readings were normalized as percentage of viable cells in the untreated control wells and the IC₅₀ values were calculated by Prism software.

FIG. 30 illustrates cytotoxicity of ADCs with linker-drug LD038 on tumor cell lines OVCAR-3. As shown in FIG. 30, all candidates (antibodies 2C5, 2C8, 13C4) have better in vitro cytotoxicity than cofetuzumab when conjugated with similar linker-payload, LD038 on human PTK7-expressing tumor cell lines. In addition, in vitro cytotoxicity of the 2C5 and 2C8 conjugates are better than that of the 13C4 and cofetuzumab conjugates.

### Example 37: Cytotoxicity Comparison of PRO1107, MMAE and b12-LD343

One day prior to adding test article (PRO1107, MMAE alone or b12-LD343 (8)), cells were harvested and plated into 96-well solid white flat bottom plates. The next day cells were exposed to the test article at concentrations from 666.67 to 0.034 nM. Plates were incubated at 37° C. for 96 h. After that, 40 μL Cell-titre Glo (CTG) per well was added to the plates with luciferase readings collected at 5 min after and analyzed by Microplate readers. All readings were normalized as percentage of viable cells in the untreated control wells and the IC₅₀ values were calculated by Prism software.

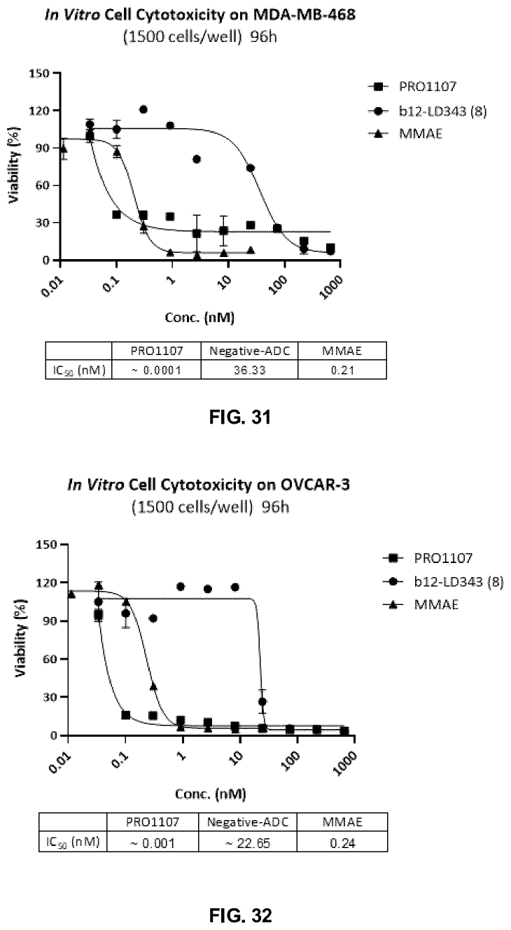

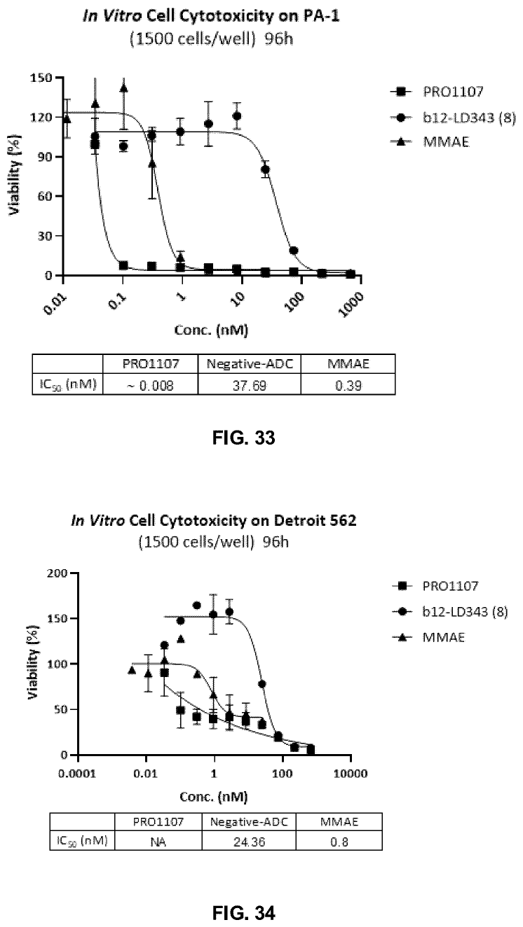

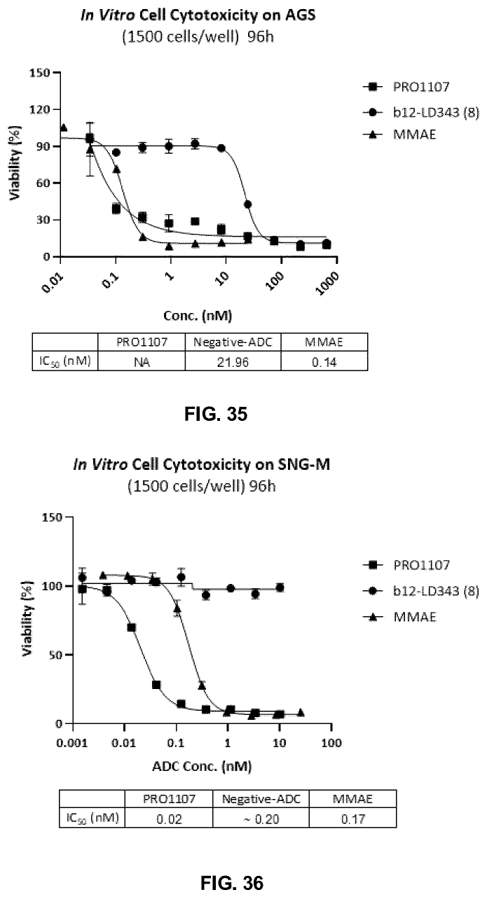

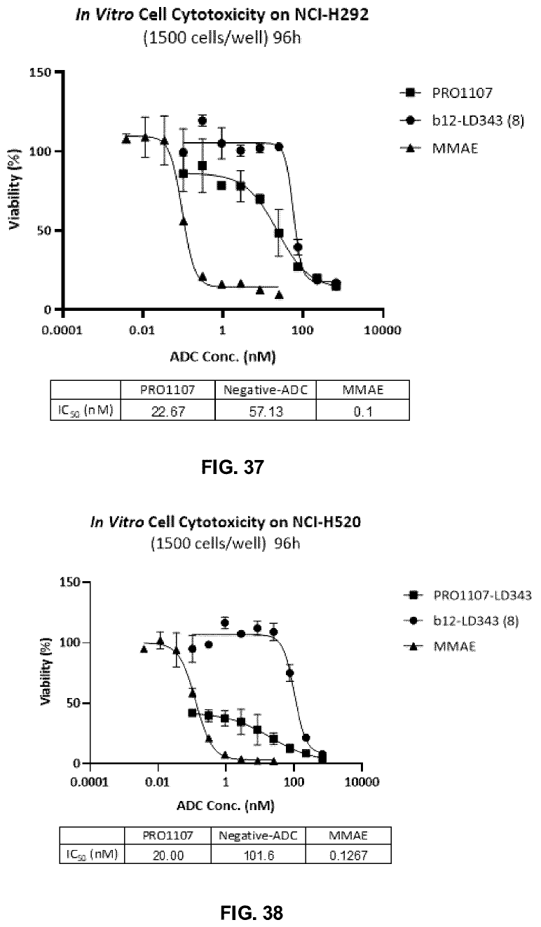

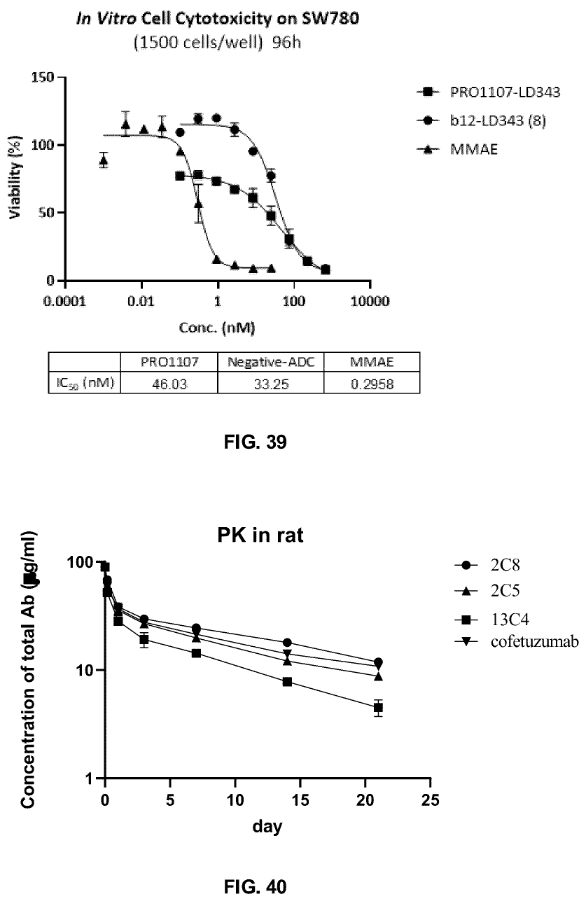

FIGS. 31-39 illustrate cytotoxicity of PRO1107, MMAE alone and b12-LD343 (8) on tumor cell lines, respectively. As shown in FIGS. 31-39, PRO1107 was remarkably potent on human PTK7-expressing tumor cell line PA-1, OVCAR-3, MDA-MB-468 and SNG-M (IC₅₀=8, 1.1, 0.14 and 20 pM, respectively), and highly potent on SW780 and Detroit 562 cells (IC₅₀=27.4, 8.2 nM, respectively). MMAE was cytotoxic on all cell lines whereas negative control-ADC (b12-LD343 (8)) showed weak cytotoxic in the assay.

### Example 38: PK Comparison in Rat

### PK Comparison Between Antibodies 2C5, 2C8, 13C4 and Cofetuzumab

2C5, 2C8, 13C4 and cofetuzumab were intravenously administered at 3 mg/kg to male Sprague Dawley rats (n=3 per group). Orbital blood was cross-sampled from each rat at 10 min, 4 h, 1d, 4d, 7d, 10d, 14d, and 21d post dosing. Total antibody (TAb) concentration of 2C5, 2C8, 13C4 and cofetuzumab in plasma determined by capturing the mAbs with human PTK7 protein and detected by goat anti-human IgG Fc (HRP), calculated using Winnonlin 8.1 software.

FIG. 40 illustrates PK of antibodies 2C5, 2C8, 13C4 and cofetuzumab in rat. As shown in FIG. 40, all candidates 2C5, 2C8, 13C4 have similar PK characteristics comparable to cofetuzumab.

### PK Comparison Between Antibody 2C8 and ADC PRO1107

2C8 and PRO1107 were intravenously administered at 3 mg/kg to male Sprague Dawley rats (n=3 per group). Orbital blood was cross-sampled from each rat at 10 min, 4 h, ₁d, 4d, 7d, 10d, 14d, and 21d post dosing. Total antibody (TAb) concentration of PRO1107 and 2C8 in plasma was determined by capturing the ADC or mAb with human PTK7 protein and detected by goat anti-human IgG Fc (HRP), calculated using Winnonlin 8.1 software.

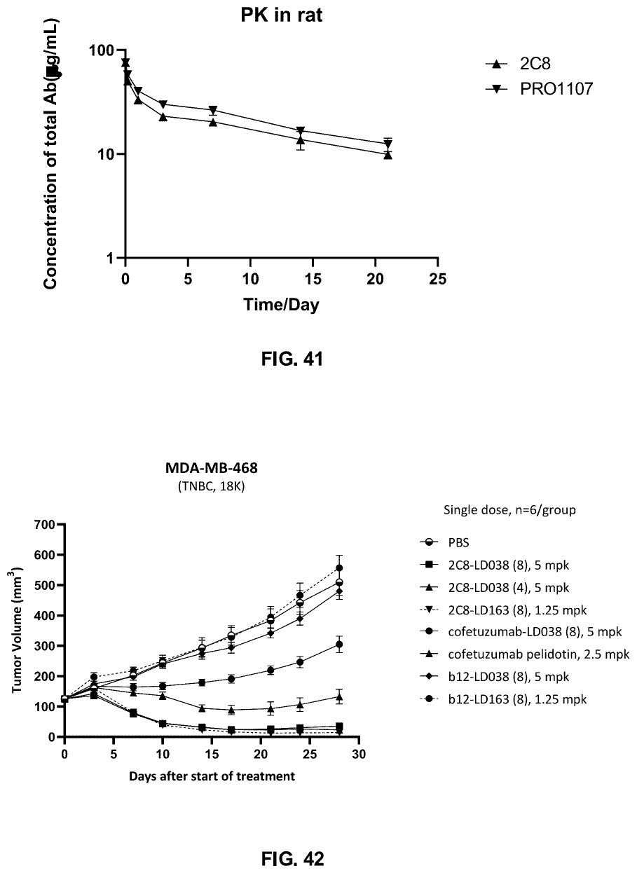

FIG. 41 illustrates PK of antibody 2C8 and ADC PRO1107 in rat. PRO1107 exhibited excellent PK characteristics that are comparable to the unconjugated parent mAb, 2C8.

### Example 39: Efficacy Comparison

### Efficacy Comparison Between 2C8 Conjugates and Cofetuzumab Conjugates in MDA-MB-468 Xenograft Model

The in vivo anti-tumor activity of 2C8 and cofetuzumab conjugates(2C8-LD038 (8), 2C8-LD038 (4), 2C8-LD163 (8), cofetuzumab-LD038 (8) and cofetuzumab pelidotin) were evaluated in tumor cell line MDA-MB-468.

MDA-MB-468 (Procell) tumor model was established by injecting 5×10⁶ cells suspended in 0.1 mL PBS mixed with Matrigel (1:1). 14 days after tumor inoculation, mice with average tumor size ˜125 mm³ were selected and assigned into 8 groups using stratified randomization (n=6 per group) based upon their tumor volumes. Mice were treated with a single (on day 0) intravenous injection of 2C8-LD038 (8), 2C8-LD038 (4) or cofetuzumab-LD038 (8) at 5 mg/kg, or 2C8-LD163 (8) at 1.25 mg/kg, or cofetuzumab pelidotin at 2.5 mg/kg.

The tumor size and body weight were measured. Animal body weight was monitored as an indirect measure of tolerability. No mice showed significant weight loss in any of the treatment groups. There were no morbidity and deaths during the treatment duration.

FIG. 42 illustrates efficacy of 2C8 conjugates and cofetuzumab conjugates in MDA-MB-468 xenograft model. As shown in FIG. 42, 2C8 had better in vivo efficacy than cofetuzumab when conjugated with similar linker-payload, LD038, in MDA-MB-468 xenograft model.

### Efficacy Comparison Between 2C8 Conjugates and Cofetuzumab Conjugates in PA-1 Xenograft Model

The in vivo anti-tumor activity of 2C8 and cofetuzumab conjugates (2C8-LD038 (8), 2C8-LD038 (4), 2C8-LD163 (8), cofetuzumab-LD038 (8) and cofetuzumab pelidotin) were evaluated in tumor cell line PA-1.

PA-1 (Procell) tumor model was established by injecting 5×10⁶ cells suspended in 0.1 mL PBS mixed with Matrigel (1:1). 18 days after tumor inoculation, mice with average tumor size ˜12⁹ mm³ were selected and assigned into 11 groups using stratified randomization (n=6 per group) based upon their tumor volumes. Mice were treated with a single (on day 0) intravenous injection of 2C8-LD038 (8), cofetuzumab-LD038 (8) at 5 mg/kg, or 2C8-LD163 (8), cofetuzumab-LD163 (8) at 2.5 mg/kg, or 2C8-vedotin (4), cofetuzumab-vedotin (4), 2C8-LD163 (4) at 5 mg/kg.

The tumor size and body weight were measured. Animal body weight was monitored as an indirect measure of tolerability. No mice showed significant weight loss in any of the treatment groups. There were no morbidity and deaths during the treatment duration.

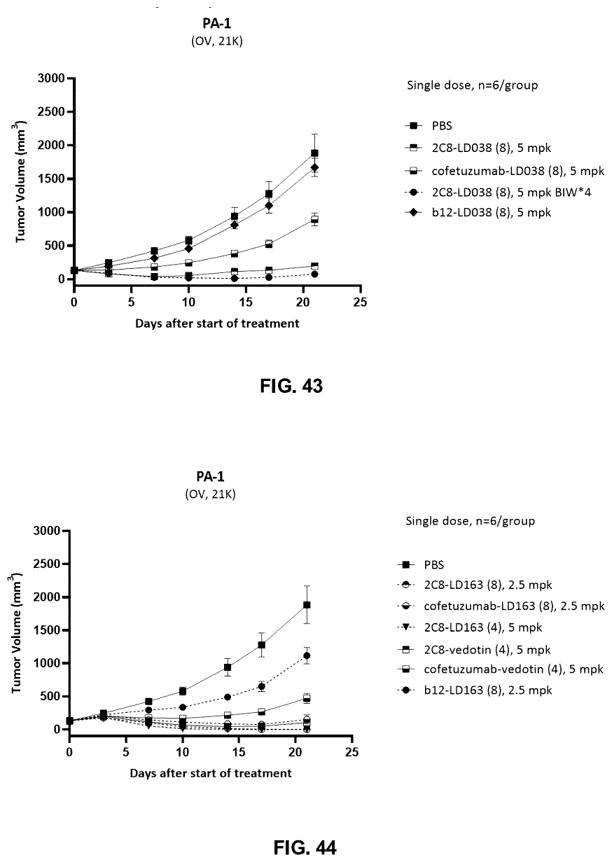

FIGS. 43 and 44 illustrate efficacy of 2C8 conjugates and cofetuzumab conjugates in PA-1 xenograft model. As shown in FIGS. 43 and 44, 2C8 conjugates have better in vivo efficacy than cofetzumab conjugates when the antibodies are conjugated with similar linker-payload, LD038, LD163 and vedotin, in PA-1 xenograft model.

### Efficacy Comparison Between PRO1107, Cofetuzumab Pelidotin and 2C8-Vedotin (4) in Various Xenograft Models

The in vivo anti-tumor activity of PRO1107, 2C8-vedotin (4), cofetuzumab pelidotin, b12-LD343 (8), and b12-vedotin (4) were evaluated in tumor cell line PA-1, OVCAR-3, MB-MDA-468, Detroit 562, SW780, RT4, AGS, KYSE-150, NCI-H292.

PA-1 (Procell) tumor model was established by injecting 5×10⁶ cells suspended in 0.1 mL PBS mixed with Matrigel (1:1). 18 days after tumor inoculation, mice with average tumor size ˜129 mm³ were selected and assigned into 11 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

MDA-MB-468 (Procell) tumor model was established by injecting 5×10⁶ cells suspended in 0.1 mL PBS mixed with Matrigel (1:1). 14 days after tumor inoculation, mice with average tumor size ˜125 mm³ were selected and assigned into 8 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

OVCAR-3 (COBIOER) tumor model was established by injecting 1×10⁷ cells suspended in 0.1 MI mixed with Matrigel (1:1). 23 days after tumor inoculation, mice with average tumor size ˜156 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

Detroit 562 (COBIOER) tumor model was established by injecting 3×10⁶ cells suspended in 0.1 mL PBS. 12 days after tumor inoculation, mice with average tumor size ˜127 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

SW780 (ATCC) tumor model was established by injecting 5×10⁶ cells suspended in 0.1 mL mixed with Matrigel (1:1). 6 days after tumor inoculation, mice with average tumor size ˜127 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

RT-4 (COBIOER) tumor model was established by injecting 5×10⁶ cells suspended in 0.1 mL mixed with Matrigel (1:1). 11 days after tumor inoculation, mice with average tumor size ˜132 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

AGS (ATCC) tumor model was established by injecting 5×10⁶ cells suspended in 0.1 mL mixed with Matrigel (1:1). 20 days after tumor inoculation, mice with average tumor size ˜119 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

KYSE-150 (COBIOER) tumor model was established by injecting 6×10⁶ cells suspended in 0.1 mL PBS. 16 days after tumor inoculation, mice with average tumor size ˜137 mm³ were selected and assigned into 2 groups using stratified randomization (n=3 per group) based upon their tumor volumes. Mice were treated with a single (on day 0) intravenous injection of PRO1107 at 1.25 mg/kg.

NCI-H292 (COBIOER) tumor model was established by injecting 5×10⁶ cells suspended in 0.1 mL mixed with Matrigel (1:1). 5 days after tumor inoculation, mice with average tumor size ˜145 mm³ were selected and assigned into 6 groups using stratified randomization (n=6 per group) based upon their tumor volumes. Mice were treated with a single (on day 0) intravenous injection of PRO1107 and b12-LD343 (8) at 1.25 mg/kg, or 2C8-vedotin (4), cofetuzumab pelidotin and b12-vedotin (4) at 2.5 mg/kg.

The tumor size and body weight were measured as described before. Animal body weight was monitored as an indirect measure of tolerability. No mice showed significant weight loss in any of the treatment groups. There were no morbidity and deaths during the treatment duration.

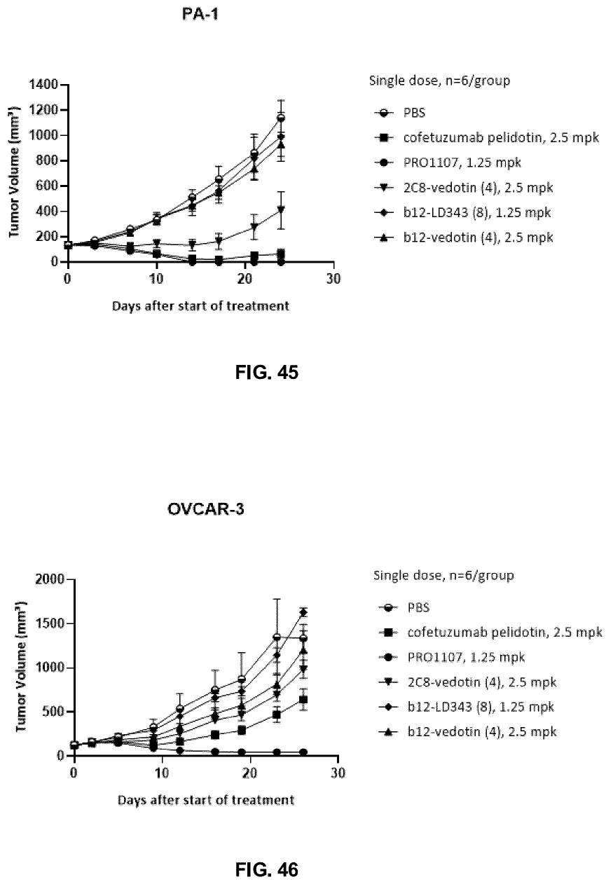

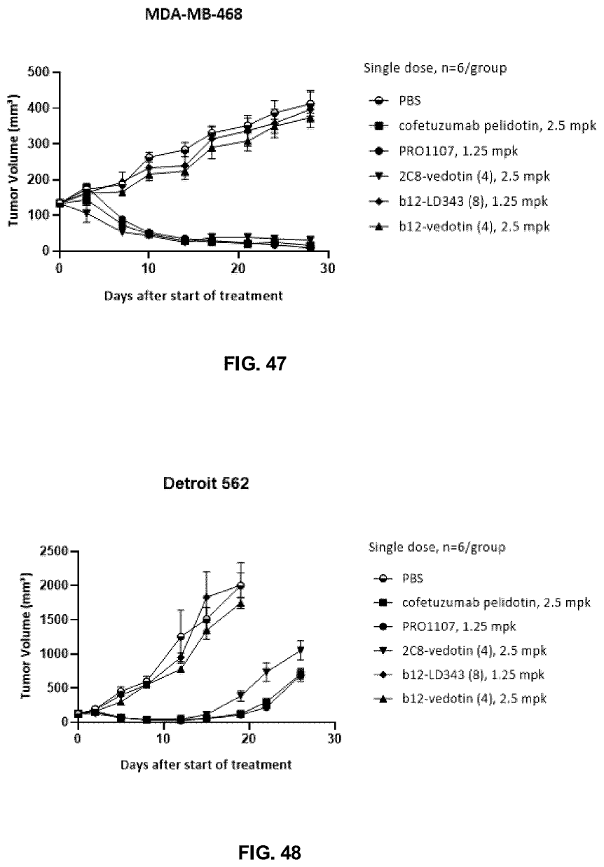

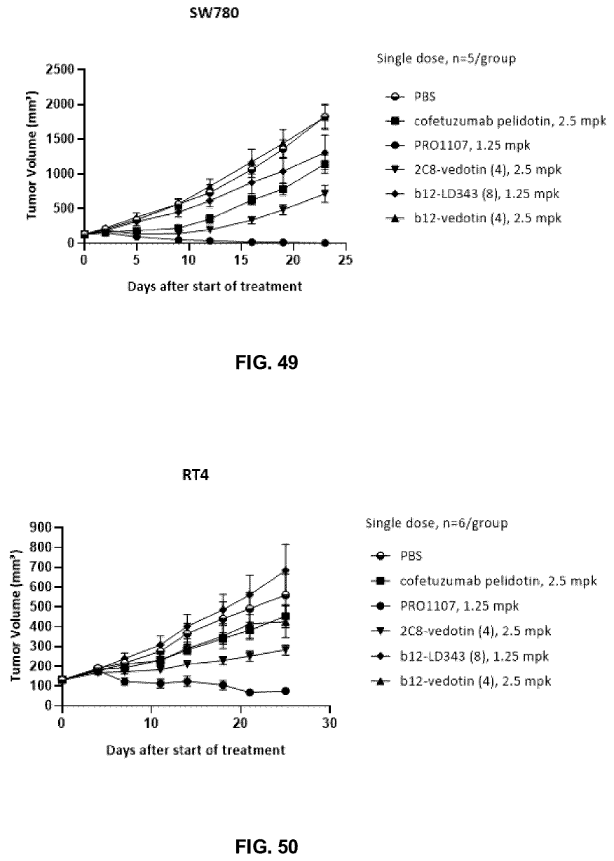

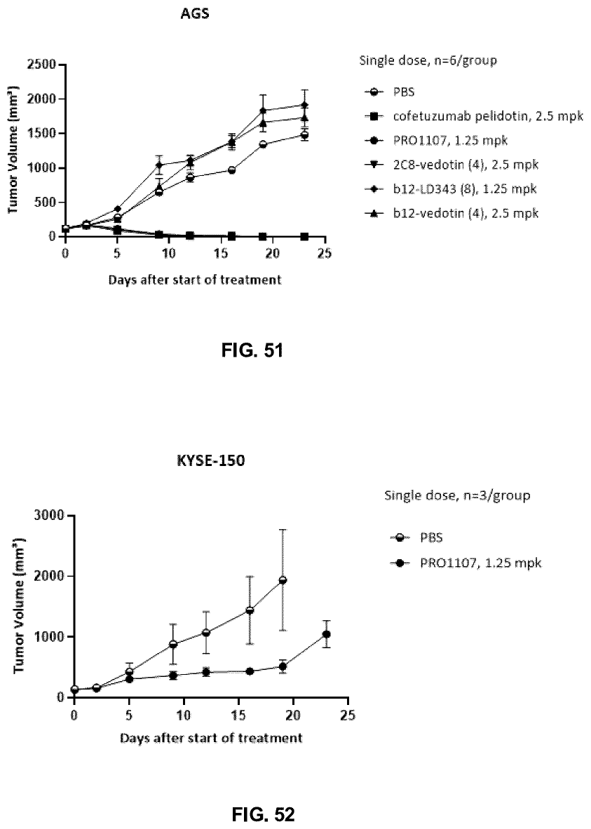

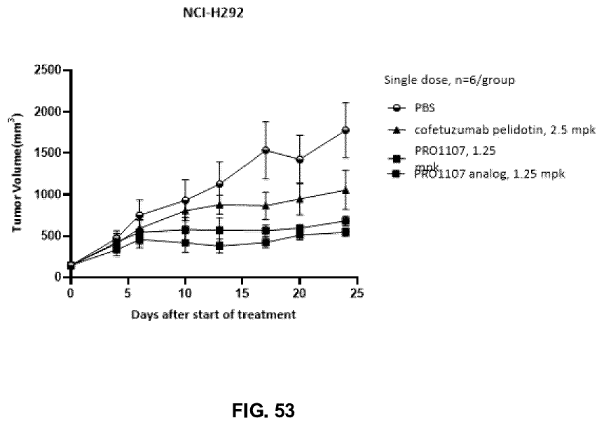

Efficacy results of PRO1107, cofetuzumab pelidotin and 2C8-vedotin (4) in various xenograft models are shown in FIGS. 45-53. As shown in FIGS. 45-53, PRO1107 showed better efficacy in vivo than cofetuzumab pelidotin and 2C8-vedotin (4) in multiple tumor models.

### Example 40: Intratumoral PK of ADCs

Observed intratumoral pharmacokinetics (PK) of ADC analytes in mice after intravenous administration of 1.5 mg/kg of PRO1107 and 3.0 mg/kg of 2C₈-vedotin (4) and cofetuzumab pelidotin single dose.

### Intratumoral PK Method:

Nude mice bearing MBA-MD-468 xenografts (average ˜250 mm³) received 1.5 mg/kg of PRO1107, 3.0 mg/kg of 2C8-vedotin (4) and 3 mg/kg of cofetuzumab pelidotin single dose via intravenous injection, respectively. Blood samples were collected at 10 min, 1, 6, 24, 72 and 168 h. Tumor samples were collected at 10 min and 6, 24, 72, and 168 h after the terminal blood sample collection, with four mice sacrificed at each time point. The collected tumor samples were weighted and added with 200 μL of N, N-dimethylformamide (DMF) buffer. Tumor samples were homogenized using a cryogenic tissue grinder (CLJXFSTPRP-CL, Shanghaijingxin) and stored in −80° C. before further analysis.

### LC-MS/MS to Quantify Free MMAE:

Waters LC-MS/MS system with electrospray ionization and triple quadrupole mass spectrometer was used (Waters/Acquity UPLC H-Class PLUS-Xevo G2-XS Qtof). The column (AQUITY UPLC® Protein BEH C4, 300 Å, 1.7 μm, 2.1×50 mm) was used. Mobile phase A is 0.1% formic acid in water and mobile phase B is 0.1% formic acid in acetonitrile. The duration of the chromatographic run was 5 min (gradient from 90% mobile phase A and 10% mobile phase B to 10% mobile phase A and 90% mobile phase B), where two MRM scans (718.5/686.5 and 718.5/152.1 amu) were monitored. Deuterated (d8) MMAE was used as an internal standard. The samples (standard, QC, plasma, and tumor homogenates) were spiked with d8-MMAE, and acetonitrile was added followed by vortex, centrifugation (15,000 g for 10 min at 4° C.), and collection of supernatants. Both analytes are finally quantitated as nanogram MMAE per gram tumor tissue.

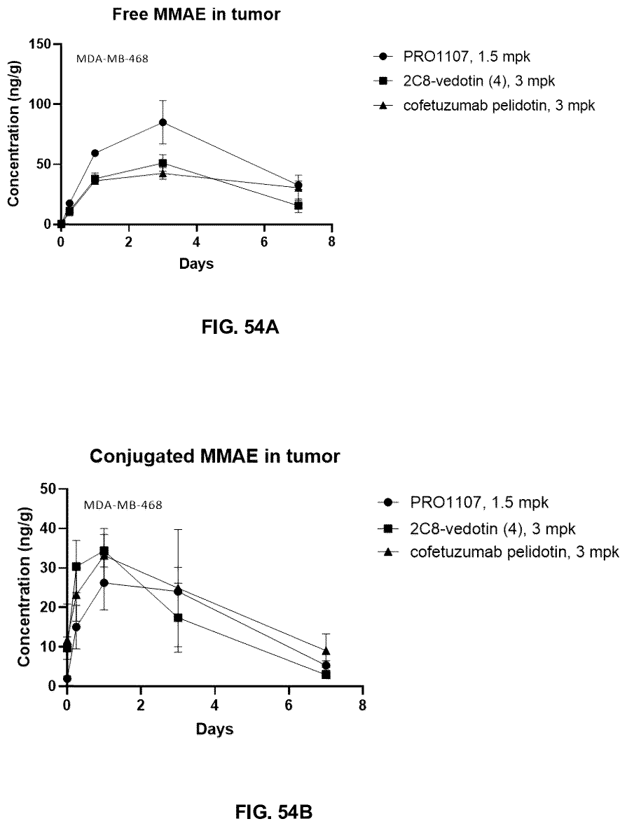

FIG. 54A and FIG. 54B are graphs illustrating free MMAE and conjugated MMAE of PRO 1107, 2C8-vedotin (4) and cofetuzumab pelidotin in tumor. As shown in FIG. 68A and FIG. 68B, PRO1107 shows better intratumoral PK in MDA-MB-468.

### Example 41: Tolerability of ADCs

### Tolerability of ADC in Rat Method:

PRO1107 were intravenously administered to Sprague Dawley rats (n=6 per group, 3 males and 3 females) at 20, 25 and 30 mg/kg, respectively. 2C8-vedotin (4) and cofetuzumab pelidotin were intravenously administered to Sprague Dawley rats (n=6 per group, 3 males and 3 females) at 15, 20 and 25 mg/kg, respectively. Animals weighed at D0 to D14 post dosing. Body weight change percentage were calculated using following formulation, RCBW (%)=(BW_{Treatment_DN}−B_{WTreatment_D0})/BW_{Treatment_D0}×100%.

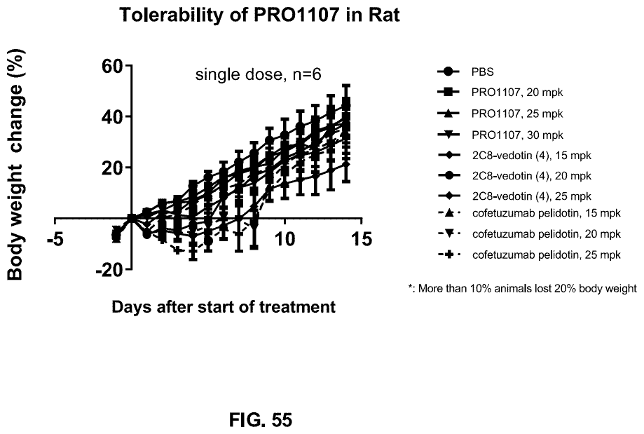

Table 6 lists dose level and survival after treatment of PRO1107, 2C8-vedotin (4) and cofetuzumab pelidotin. FIG. 55 shows body weight change of rats after treatment of PRO1107, 2C8-vedotin (4) and cofetuzumab pelidotin. Maximum tolerated dose (MTD) of PRO1107 (at DAR8), 2C8-vedotin (at DAR4) and cofetuzumab pelidotin (at DAR4) were approximately 30, 15 and 15 mg/kg, respectively, suggesting a 4-fold increase in tolerated drug load. PRO1107 shows better tolerability than 2C8-vedotin (4) and cofetuzumab pelidotin in rat.

TABLE 6
Dose level and survival after treatment

| Treatment | Dose level | Survival |
|---|---|---|
| PBS | 0 mg/kg | 6/6 |
| PRO1107 | 20 mg/kg | 6/6 |
| | 25 mg/kg | 6/6 |
| | 30 mg/kg | 6/6 |
| 2C8-vedotin (4) | 15 mg/kg | 6/6 |
| | 20 mg/kg | 5/6 |
| | 25 mg/kg | 5/6 |
| cofetuzumab pelidotin | 15 mg/kg | 6/6 |
| | 20 mg/kg | 2/6 |
| | 25 mg/kg | 2/6 |

### Example 42: Toxicity of PRO1107

Pilot Toxicity Study: Tolerated at 9 mg/kg

Monkeys (n=2 per group) were treated with 6, 9 mg/kg PRO1107, IV, Q3W*2. Table 7 shows toxicity results of PRO1107.

TABLE 7

| | Clinical | | Clinical | |
|---|---|---|---|---|
| Test Article | Observations | Hematology | Chemistry | Histopathology |
| PRO1107 | Soft feces, | ↓: WBC; LYMPH; | ↑: AST | Bone marrow; |
| (6 mg/kg) | minimum | NEUT; RETIC; | ↓: ALB; | spleen |
| | Skin swelling | RBC; HGB; HCT, | A/G | |
| | & erythema, | mostly recovered | | |
| | mild | at D22 | | |
| PRO1107 | Body weight | ↓: WBC; LYMPH; | ↑: AST | Bone marrow; |
| (9 mg/kg) | & food | NEUT; RETIC; | ↓: ALB; | spleen; |
| | consumption | RBC; HGB; HCT, | A/G | thymus, |
| | decrease, | mostly recovered | | administration |
| | minimum | at D22 | | site |
| | Soft feces, | | | |
| | minimum | | | |
| | Skin swelling | | | |
| | & erythema, | | | |
| | mild | | | |
| | Skin lesions, | | | |
| | mild to | | | |
| | extreme | | | |

As shown in Table 7, decreased erythropoiesis, leukocyte, albumin, and increased AST were observed at doses >6 mg/kg. Target organs include bone marrow, spleen, skin, and thymus. PRO1107 exhibited a very favorable toxicity profile in cynomolgus monkeys.

### Example 43: Hydrophilicity of PRO1107

Hydrophobic Interaction Chromatogram (HIC) is a valuable technique in the development and characterization of ADCs because it allows for the separation and analysis of the various components that make up these complex molecules, including the antibody, linker, and cytotoxic drug, based on their hydrophobic properties. Retention times of PRO1107 (8) and cofetuzumab pelidotin (4) were measured by HIC-HPLC.

HIC-HPLC conditions: Column: TSKgel Butyl-NPR, 2.5 μm, 4.6 mm×100 mm. Column temperature: 25±2° C. UV: 280 nm. Mobile phase: A: 50 mM PB, 1.5M (NH4)₂SO₄, pH=7.0; B: 50 mM PB (pH=7.0): isopropyl alcohol=75:25 (v:v). Flow rate:0.8 mL/min. Gradient elution: 0 min→25 min (0% B→100% B), 25 min→26 min (100% B→0% B), 26 min→30 min (0% B).

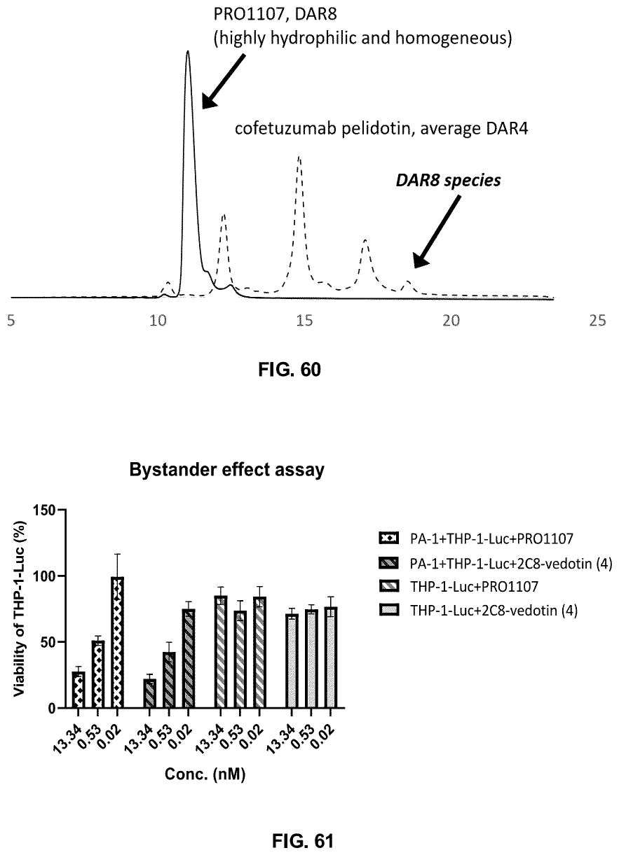

FIG. 60 shows the hydrophilicity of PRO1107. As shown in FIG. 60, at the position of DAR8, the relative retention time of PRO1107 was shorter than that of DAR8 species of corresponding MC-VC-PAB-PF06380101-based ADC, cofetuzumab pelidotin. PRO1107 exhibits higher hydrophilicity and homogeneity.

### Example 44: Bystander Effect of PRO1107

One day prior to adding ADC, target-positive cell line PA-1 and target-negative cell line THP-1-Luc were harvested and seeded into 96-well black clear flat bottom plates at ratio of 1:3.

The next day mixed cells were exposed to the PRO1107 and 2C8-vedotin (4) at 13.24, 0.53 and 0.02 nM, respectively. Plates were incubated at 37° C. for 96 h. After that, 80 μL of Bio-Glu per well was added into the plates. Incubate at room temperature for 5 minutes. Measure luminescence using a multimode plate-reader. All readings were normalized as percentage of viable cells in the untreated control wells and the IC50 values were calculated by Prism software.

Cell viability was evaluated 4 days after treatment using the Bio-Glo™ Luciferase Assay (Promega Corp). All readings were normalized as percentage of viable cells in the untreated control wells and the IC₅₀ values were calculated.

FIG. 61 shows the bystander effect of PRO1107 and vedotion based ADCs in cell lines PA-1 and THP-1-Luc, respectively. PRO1107 shows bystander killing effect in different cell lines.

### Example 45: Efficacy of LD343 Based ADCs and Vedotin Based ADCs

PA-1 (Procell) tumor model was established by injecting 5×10⁶ cells suspended in 0.1 mL PBS mixed with Matrigel (1:1). 18 days after tumor inoculation, mice with average tumor size ˜129 mm³ were selected and assigned into 11 groups using stratified randomization (n=6 per group) based upon their tumor volumes. Mice were treated with a single (on day 0) intravenous injection of mAb-LD343 (8) at 1.25 mg/kg, or mAb-vedotin (4) (mAb refers to 2C8 in PRO1107) at 2.5 mg/kg.

OVCAR-3 (COBIOER) tumor model was established by injecting 1×10⁷ cells suspended in 0.1 MI mixed with Matrigel (1:1). 23 days after tumor inoculation, mice with average tumor size ˜156 mm³ were selected and assigned into 6 groups using stratified randomization (n=6 per group) based upon their tumor volumes. Mice were treated with a single (on day 0) intravenous injection of mAb-LD343 (8) at 1.25 mg/kg, or mAb-vedotin (4) at 2.5 mg/kg (mAb refers to 2C8 in PRO1107).

Detroit 562 (COBIOER) tumor model was established by injecting 3×10⁶ cells suspended in 0.1 mL PBS. 12 days after tumor inoculation, mice with average tumor size ˜127 mm³ were selected and assigned into 6 groups using stratified randomization (n=6 per group) based upon their tumor volumes. Mice were treated with a single (on day 0) intravenous injection of mAb-LD343 (8) at 1.25 mg/kg, or mAb-vedotin (4) at 2.5 mg/kg (mAb refers to 2C8 in PRO1107).

SW780 (ATCC) tumor model was established by injecting 5×10⁶ cells suspended in 0.1 mL mixed with Matrigel (1:1). 6 days after tumor inoculation, mice with average tumor size ˜127 mm³ were selected and assigned into 6 groups using stratified randomization (n=6 per group) based upon their tumor volumes. Mice were treated with a single (on day 0) intravenous injection of mAb-LD343 (8) at 1.25 mg/kg, or mAb-vedotin (4) at 2.5 mg/kg (mAb refers to 2C8 in PRO1107).

The tumor size and body weight were measured. Animal body weight was monitored as an indirect measure of tolerability. No mice showed significant weight loss in any of the treatment groups. There were no morbidity and deaths during the treatment duration.

Pharmacokinetic and pharmacodynamic analysis of LD343 and vedotin based ADCs in various cell-line derived xenograft (CDX) models. Mice inoculated with tumor cells were intravenously treated with mAb-LD343 (1.25 mpk) or mAb-vedotin (4) (2.5 mpk) on day 0. All studies were single-dose treatment at the specified doses (n=5˜6 per treatment group).

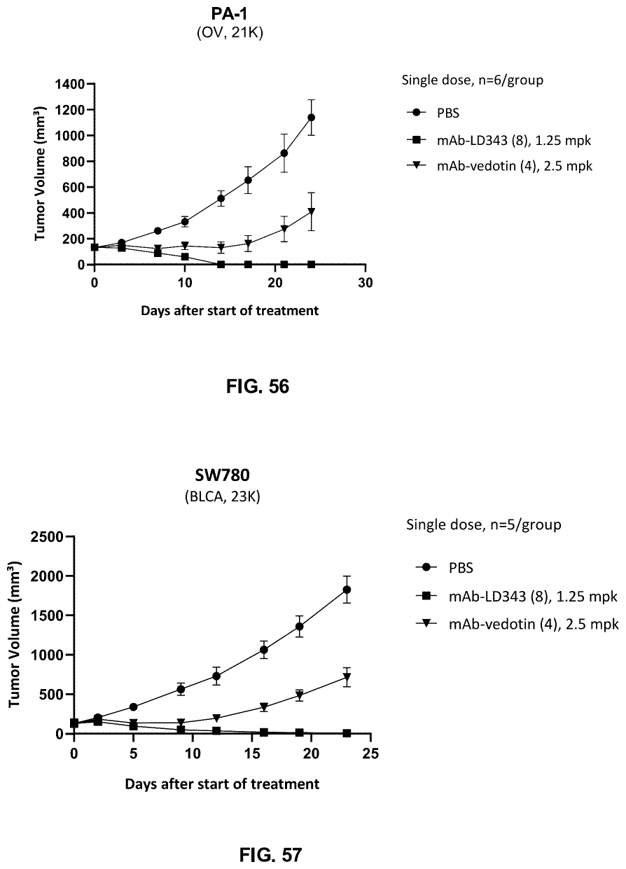

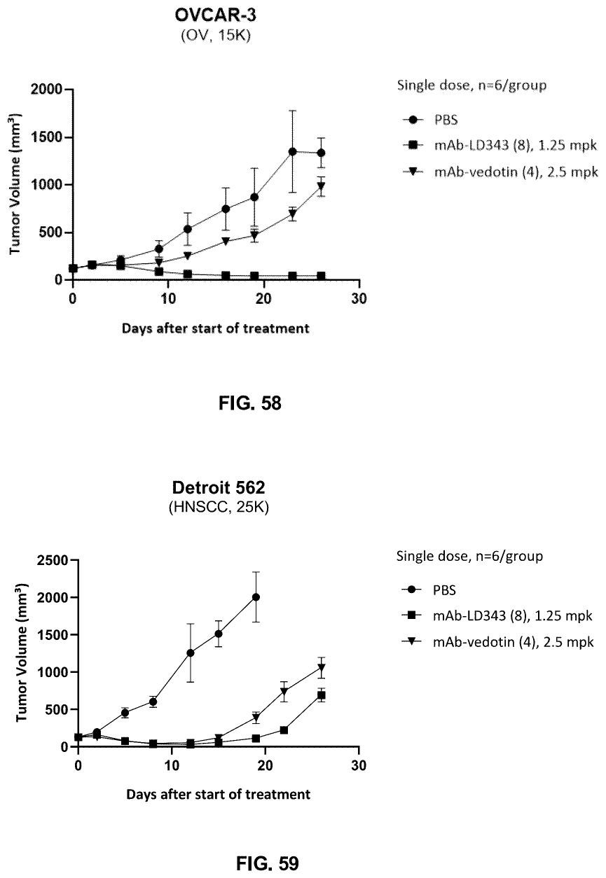

FIGS. 56-59 show antitumor activity of single dose of 2C8-LD343 (8) and 2C8-vedotin (4) in PA-1, SW780, OVCAR-3 and Detroit 562 xenograft models. None of the ADC-treated animals exhibited appreciable weight loss or apparent distress (not shown). LD343 based ADCs show better efficacy compared to vedotin based ADCs.

### Example 46: Plasma Stability of LD343 Based ADCs

### Construction of Xenograft Model:

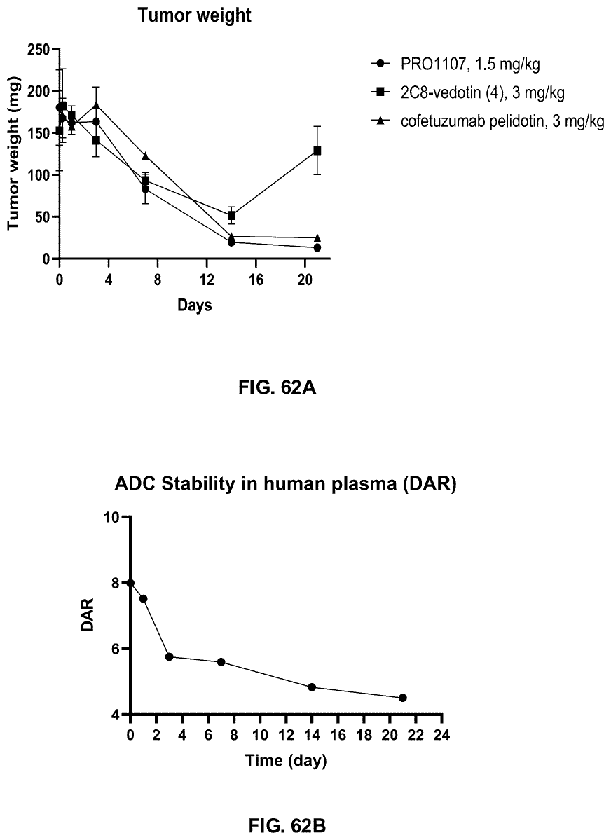

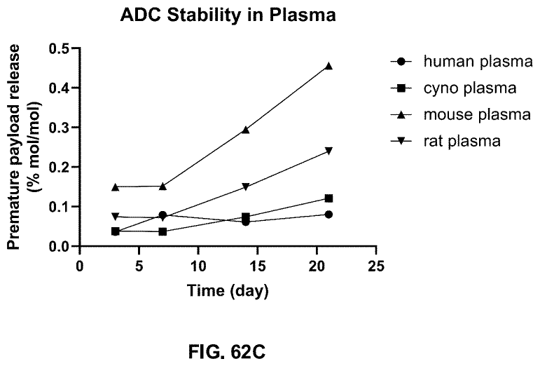

Nude mice bearing MBA-MD-468 xenografts (average ˜250 mm³) received 1.5 mg/kg of PRO1107 3.0 mg/kg of 2C₈-vedotin (4) and 3 mg/kg of cofetuzumab pelidotin single dose via intravenous injection, respectively. Blood samples were collected at 10 min, 1 h, 6 h, 24 h, 72 h and 168 h. Tumor samples were collected at 10 min and 6 h, 24 h, 72 h, and 168 h after the terminal blood sample collection, with four mice sacrificed at each time point. The collected tumor samples were weighted at each time point. FIG. 62A is a graph illustrating tumor weight change of nude mice after treatment of PRO1107, 2C8-vedotin (4) and cofetuzumab pelidotin.

Stability of PRO1107 in plasma in a 21-day incubation at 37° C. was measured.

FIG. 62B shows the change of DAR value of PRO1107 in human plasma. FIG. 62C shows the payload (MMAE) release in human, cyano, mouse and rat plasma after treatment of PRO1107. LD343 based ADC shows good stability in human plasma, cyano plasma, mouse plasma and rat plasma.

### DAR Value Measurement Method:

PRO1107 was mixed with various types of plasma (human, cyano, rat, mouse) with a final concentration of 150 μg/mL and incubated at 37° C. for 10 min (D0), 24 h (D1), 72 h (D3), 168 h (D7), 336 h (D14) and 504 h (D21) to evaluate its stability in the plasma. Samples after deglycosylation and reduction were analyzed by Waters/Acquity UPLC H-Class PLUS-Xevo G2-XS QTof system equipped with an Agilent PLRP-S1, 5 μm, 1000A, 2.1×50 mm column. The mobile phase A consisted of 0.1% FA in water and mobile phase B consisted of 0.1% FA in ACN. The gradient initiated from 25% B to 60% B in 8 min, ramped to 25% B in 0.1 min and held on until 10 min. The flow rate was 0.4 mL/min. The column temperature was set at 50° C. and the sample room temperature was 8° C. UV absorbance at 280 nm was collected. The injection volume used was 10 μL. LC-MS data was collected in sensitivity mode. The capillary voltage was set to 3 kV. The cone voltage was set to 120 V. The source temperature was set at 150° C. The desolvation temperature was set at 450° C. The cone gas flow was set at 50 L/h. The desolvation gas flow was set at 800 L/h. The scan range was set from 400 to 5000 m/z with scan rate 1 s. The data was processed using Waters Unifi microsoft. The input m/z range was set from 1000 to 2500 m/z with corresponding output range from 20000-100000 Da. The deconvolution start peak width was set as 0.2 and the end peak width was 0.5. The charge carrier was hydrogen. The maximum number of iterations was set to 15. Minimum left intensity and right intensity was set at 30%. The Dar values were calculated following the below equation:

$$DAR = 2 \times {\sum }_{i = 0}^{m} i \times [ Drug - Load Distribution L ( i ) ] + 2 \times {\sum }_{j = 0}^{n} j \times [ Drug - Load Distribution H ( i ) ] ,$$

Wherein M denotes the drug load of light chain and N denotes the drug load of heavy chain.

### LC-MS/MS to Quantify Free MMAE

PRO1107 was mixed with various types of plasma mentioned above with a final concentration of 150 μg/mL and incubated at 37° C. for 10 min (DO), 24 h (D1), 72 h (D3), 168 h (D7), 336 h (D14) and 504 h (D21) to evaluate its stability in the plasma. Samples were stored at −80° C. after incubation, if not used immediately. The MMAE-D8 internal standard was added and the samples were processed by protein precipitation and analyzed by LC-MS/MRM. The samples (standard, QC, Blank sample, sample) were spiked with d₈-MMAE in 100% acetonitrile, followed by the vortexing, centrifugation (15,000 g for 10 min at 4° C.), and collection of supernatants added with 0.1% FA in H₂O. Vortex for injection. Waters LC-MS/MS system with electrospray ionization and Xevo® G2-XS Q Tof was used. The column (ACQUITY UPLC Protein C4, 1.7 μm, 2.1*50 mm) was used with the aqueous phase as 0.1% FA in H₂O and the organic phase (0.1% FA in ACN). The duration of the chromatographic run was 5.0 min, where two MRM scans (718.5113/686.4851) were monitored. Deuterated (d8) MMAE was used as an internal standard (726.5615/694.5353).

### Intratumor Free Payload Method

Nude mice bearing MBA-MD-468 xenografts (average ˜250 mm³) received 1.5 mg/kg of PRO1107 3.0 mg/kg of 2C8-vedotin (4) and 3 mg/kg of cofetuzumab pelidotin (4) single dose via intravenous injection, respectively. Blood samples were collected at 10 min, 1, 6, 24, 72 and 168 h. Tumor samples were collected at 10 min and 6, 24, 72, and 168 h after the terminal blood sample collection, with four mice sacrificed at each time point. The collected tumor samples were weighted at each time point, and added with 200 μL of N,N-dimethylformamide (DMF) buffer, then centrifugation (15,000 g for 10 min at 4° C.) and collection of supernatants. The samples (standard, QC, Blank sample, tumor homogenates) were spiked with d8-MMAE solution, Vortex for injection. Waters LC-MS/MS system with electrospray ionization and Xevo® G2-XS Q Tof was used. For UPLC, the column (ACQUITY UPLC Protein C4, 1.7 μm, 2.1*50 mm) was used with the aqueous phase(Phase A:0.1% FA in H₂O) and the organic phase (Phase B:0.1% FA in ACN). The duration of the chromatographic run was 5.0 min, with gradient as 0-3 min: 90% A-10% A; 3-3.1 min: 10% A-90% A, 3.1-5 min: 90% A-90% A. For MS, The scan range was set from 100 to 1200 m/z. Two MRM scans (718.5113/686.4851 for MMAE) were monitored. Deuterated (d8) MMAE was used as an internal standard (726.5615/694.5353). Linear and weight factor (1/X²) was used for qualification regression. Standard curve range from 0.1 to 100 ng/mL.

### Example 47: Efficacy of PRO1107 in Cell-Derived Xenograft (CDX) Models

Appropriate number of cells suspended in either Matrigel/PBS (1:1), or PBS, were used for inoculation subcutaneously into female Balb/c nude NDG or NOD/SCID mice. At day 6-36 after tumor inoculation, mice with average tumor size of 119.43-160.23 mm³ were selected and assigned into treatment groups (n=3-6 per group) using stratified randomization based off their tumor volumes. Treatment with intravenous injection of the PRO1107 or vehicle control initiated on the same day of randomization in the single dose model. Tumor size was measured twice a week using Studylog software. Animal body weight was monitored as an indirect measure of toxicity. No morbidity or deaths were observed in any of the treatment groups during the treatment duration.

Best tumor response (%) was calculated by the following equation and evaluated.

$$\frac{\frac{Td - T 0}{T 0} \times 100}{N} \pm SEM$$

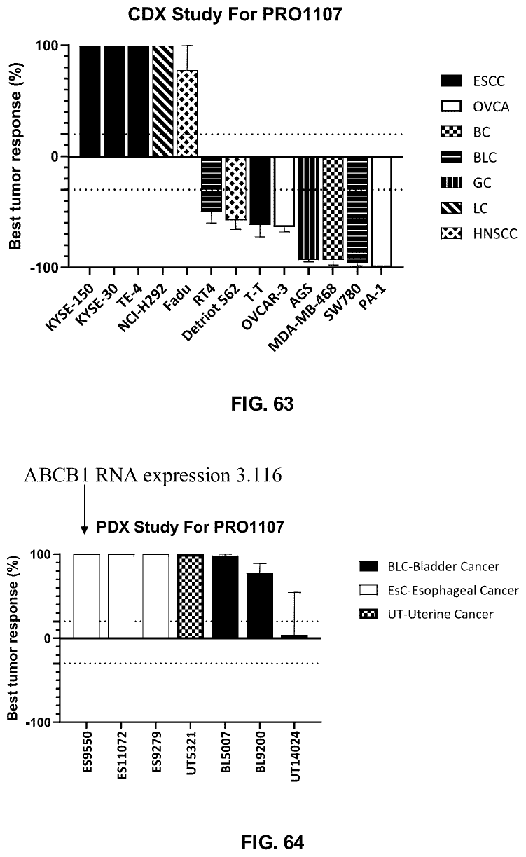

Td and T0 are the tumor volumes of treatment group on effective day and a treatment start day, respectively; N is the number of animals in group. When calculated response >100, the normalized value is setting as 100 for SEM calculation and graphing. The results of efficacy of PRO1107 in various cancer cell lines are as shown in Table 8 and FIG. 63.

TABLE 8

| CDX Model | Model Type | Best Tumor Response |
|---|---|---|
| KYSE-150 | ESCC | 100.00 |
| KYSE-30 | ESCC | 100.00 |
| TE−4 | ESCC | 100.00 |
| NCI-H292 | LC | 100.00 |
| Fadu | HNSCC | 77.61 |
| RT4 | BLCA | −50.35 |
| Detriot 562 | HNSCC | −57.58 |
| T-T | ESCC | −61.71 |
| OVCAR-3 | OV | −63.62 |
| AGS | GC | −93.37 |
| MDA-MB-468 | BC | −93.16 |
| SW780 | BLCA | −96.28 |
| PA-1 | OV | −100.00 |

### Example 48: Efficacy of PRO1107 in Patient-Derived Xenograft (PDX) Models

Tumor fragments from stock mice were harvested and used for inoculation subcutaneously into female Balb/c nude or NOD/SCID mice. After tumor inoculation, mice with average tumor size of 134.11-157.04 mm³ were selected and assigned into treatment groups (n=2-3 per group) using randomization based on “Matched distribution” method. Treatment with intravenous injection of the PRO1107 or vehicle control initiated on the same day of randomization in the single dose model. Tumor size was measured twice a week using StudyDirector™ software. Animal body weight was monitored as an indirect measure of toxicity.

Best tumor response (%) was calculated by the following equation and evaluated.

$$\frac{\frac{Td - T 0}{T 0} \times 100}{N} \pm SEM$$

Td and T0 are the tumor volumes of treatment group on effective day and a treatment start day, respectively; N is the number of animals in group. When calculated response >100, the normalized value is setting as 100 for SEM calculation and graphing. The results of efficacy of PRO1107 in various PDX models are as shown Table 9 and FIG. 64.

TABLE 9

| PDX Model | Model Type | Best Tumor Response |
|---|---|---|
| ES9550 | Esophageal cancer | 100.00 |
| ES11072 | Esophageal cancer | 100.00 |
| ES9279 | Esophageal cancer | 100.00 |
| UT5321 | Uterine cancer | 100.00 |
| BL5007 | Bladder cancer | 98.36 |
| BL9200 | Bladder cancer | 78.11 |
| UT14024 | Uterine cancer | 4.00 |

Tumor growth inhibition (%) was evaluated and compared to a control group treated with cofetuzumab pelidotin. The results are as shown in Table 10.

TABLE 10

| | | | | Tumor growth | |
|---|---|---|---|---|---|
| | | | | inhibition compared to | |
| | PDX | PTK7 | | control group (%) | |
| | model | RNA | H- | | cofetuzumab |
| Model type | (IHC) | expression | Score | PRO1107 | pelidotin |
| BL-Bladder | BL9200 | 3.731 | 155.7 | 54.56 | 58.88 |
| cancer | BL500 | 5.688 | 235.28 | 23.62 | −13.49 |
| ES- | ES955 | 7.353 | 270.2 | 45.73 | 4.46 |
| Esophageal | ES1107 | 6.265 | 261.55 | 48.28 | 22.40 |
| cancer | ES927 | 0.146 | 72.68 | −8.55 | −33.35 |
| UT-Uterine | UT5321 | 6.348 | 243.72 | 54.39 | 2.31 |
| cancer | UT14024 | 6.03 | 266.45 | 87.53 | 75.40 |

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

SEQUENCE LISTING

SEQ ID NO: 1
QVQLVQSGAEVKKPGASVKVSCKASGYTFTDYYINWVRQAPGQGLEWMGDINPYSGGPVYNQKF
MARVTMTVDKSINTAYMELSRLRSDDTAVYYCARGDYYGSNYNYWGQGTLVTVSS
SEQ ID NO: 2
EIVLTQSPATLSVSPGERATLSCRASQNIGTSIHWYQQKPGQAPRLLIKFASESISGIPARFSGSGSG
TEFTLTISSLQSEDIAVYYCQQSNNWPYTFGQGTKLEIK
SEQ ID NO: 3 GYTFTDYY
SEQ ID NO: 4 INPYSGGP
SEQ ID NO: 5 ARGDYYGSNYNY
SEQ ID NO: 6 QNIGTS
SEQ ID NO: 7 QQSNNWPYT
SEQ ID NO: 8
QVQLVESGGGLVKPGGSLRLSCAASGFAFSTYDMFWIRQAPGKGLEWVSTISSGGGYTYYPGSVK
GRFTISRDNAKNSLYLQMNSLRAEDTAVYYCVRPLLRDSYFYFDVWGQGTMVTVSS
SEQ ID NO: 9
EIVMTQSPATLSVSPGERATLSCRASSSVSSSYLHWYQQKPGQAPRLLIFRTSNLASGIPARFSGSG
SGTEYTLTISSLQSEDAAVYYCQQYSGYPLTFGQGTKLEIK
SEQ ID NO: 10 GFAFSTYD
SEQ ID NO: 11 ISSGGGYT
SEQ ID NO: 12 VRPLLRDSYFYFDV
SEQ ID NO: 13 SSVSSSY
SEQ ID NO: 14 QQYSGYPLT
SEQ ID NO: 15
QIQLVQSGAEVKKPGASVKVSCKASGYTFTTYGMSWVRQAPGQGLEWMGWINTHSGVPTYVDEF
KGRVTMTLDTSTSTAYMELSSLRSEDTAVYYCARSPFDYGSRGAWFVYWGQGTTVTVSS
SEQ ID NO: 16
DVVMTQSPDSLAVSLGERATINCRSSQSIVHNSGDTYLEWYQQKPGQPPKLLIYKVSNRFPGVPDR
FSGSGSGTDFTLTISSLQAEDLAVYYCFQGSHVPWTFGGGTKVEIK
SEQ ID NO: 17 GYTFTTYGMS
SEQ ID NO: 18 INTHSGVP
SEQ ID NO: 19 ARSPFDYGSRGAWFVY
SEQ ID NO: 20 QSIVHNSGDTY
SEQ ID NO: 22 FQGSHVPWT
SEQ ID NO: 23 GYTFTTYG
SEQ ID NO: 24 INTHSGVP
SEQ ID NO: 25 ARSPFDYGSRGAWFVY
SEQ ID NO: 26 QSIVHNSGDTY
SEQ ID NO: 27 FQGSHVPWT
SEQ ID NO: 28 TYGMS
SEQ ID NO: 29 WINTHSGVPTYVDEFKG
SEQ ID NO: 30 SPFDYGSRGAWFVY
SEQ ID NO: 31 RSSQSIVHNSGDTYLE
SEQ ID NO: 32 KVSNRFP
SEQ ID NO: 33 FQGSHVPWT
SEQ ID NO: 34
EVQLLSSGPELVTPGASVKISCKASGYTFTDYYINWLKQSHGKSLEWIGDINPNSGGPVYNQKFMA
KATLTVDKTSNTAYMELRSLTSEDSAVYYCARGDYYGSNYNYWGQGTTLTVSS
SEQ ID NO: 35
DILLTQSPAILSVSPGERVSFSCRASQNIGTSIHWYQQRTNGSPRLLIKFASESISGIPSRFSGSGSGT
DFALTINSVESEDIADYYCQQSNNWPYTFGGGTKLEIK
SEQ ID NO: 36 GYTFTDYY
SEQ ID NO: 37 INPNSGGP
SEQ ID NO: 38 ARGDYYGSNYNY
SEQ ID NO: 39 QNIGTS
SEQ ID NO: 40 QQSNNWPYT
SEQ ID NO: 41
EVKLVESGGGLVKPGGSLKLSCAASGFAFSTYDMFWFRQTPEKRLEWVATISSGGGYTYYPGSVK
GRFTISRDNARNTLYLQMSSLRSEDTALYYCVRPLLRDSYFYFDVWGAGTTVTVSS
SEQ ID NO: 42
DIVMTQSPAIMSTSPGEKVTMTCRASSSVSSSYLHWYQQKSGASPKLWIFRTSNLASGVPARFSGS
GSGTSYSLTISSVEAEDAATYYCQQYSGYPLTFGAGTKLELK
SEQ ID NO: 43 GFAFSTYD
SEQ ID NO: 44 ISSGGGYT
SEQ ID NO: 45 VRPLLRDSYFYFDV
SEQ ID NO: 46 SSVSSSY
SEQ ID NO: 47 QQYSGYPLT
SEQ ID NO: 48
QIQLVQSGPELKKPGETVKISCKASGYTFTTYGMSWVKQAPGKGLKWMGWINTHSGVPTYVDEFK
GRSAFSLETSASTAYLQINNLKNEDTATYFCARSPFDYGSRGAWFVYWGQGTLVTVSS
SEQ ID NO: 49
DVLMTQTPLSLPVSLGDQASISCRSSQSIVHNNGDTYLEWYLQKPGQSPKLLIYKVSNRFPGVPDRF
SGSGSGTDFTLKISRVEAEDLGLYYCFQGSHVPWTFGGGTKLEIK
SEQ ID NO: 50 GYTFTTYG
SEQ ID NO: 51 INTHSGVP
SEQ ID NO: 52 ARSPFDYGSRGAWFVY
SEQ ID NO: 53 QSIVHNNGDTY
SEQ ID NO: 55 FQGSHVPWT
SEQ ID NO: 56
ASTKGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 57
RTVAAPSVFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 58 GGGGS
SEQ ID NO: 59 HHHHHH
SEQ ID NO: 60 GGFG
SEQ ID NO: 61 LPXTG

1. A binding agent comprising:
a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of:
SEQ ID NO: 17, SEQ ID NO: 18, SEQ ID NO: 19, SEQ ID NO: 20, KVS and SEQ ID NO: 22, respectively;
SEQ ID NO: 3, SEQ ID NO: 4, SEQ ID NO: 5, SEQ ID NO: 6, FAS and SEQ ID NO: 7, respectively; and
SEQ ID NO: 10, SEQ ID NO: 11, SEQ ID NO: 12, SEQ ID NO: 13, RTS and SEQ ID NO: 14, respectively.

2. The binding agent of claim 1, wherein the VH and VL regions have amino acid sequences that are selected from the pairs of amino acid sequences set forth in the group consisting of:
a. SEQ ID NO: 15 and SEQ ID NO: 16, respectively;
b. SEQ ID NO: 1 and SEQ ID NO: 2, respectively; and
c. SEQ ID NO: 8 and SEQ ID NO: 9, respectively.

3. The binding agent of claim 1, wherein the VH and VL regions have amino acid sequences that are selected from the pairs of amino acid sequences set forth in the group consisting of:
a. SEQ ID NO: 15 and SEQ ID NO: 16, respectively;
b. SEQ ID NO: 1 and SEQ ID NO: 2, respectively; and
c. SEQ ID NO: 8 and SEQ ID NO: 9, respectively.
wherein the heavy and light chain framework regions are optionally modified with from 1 to 8 amino acid substitutions, deletions or insertions in the framework regions.

4. The binding agent of claim 1, wherein HCDR1, HCDR2 and HCDR3 and LCDR1, LCDR2 and LCDR3 have the amino acid sequences set forth in SEQ ID NO: 17, SEQ ID NO: 18, SEQ ID NO: 19, SEQ ID NO: 20, KVS and SEQ ID NO: 22, respectively.

5. The binding agent of claim 1, wherein the framework regions are human framework regions.

6. The binding agent of claim 1, wherein the binding agent is an antibody or an antigen-binding portion thereof.

7. The binding agent of claim 1, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

8.-11. (canceled)

12. The binding agent of claim 1, further comprising an IgG1 constant region having the amino acid sequence set forth in SEQ ID NO: 56.

13.-14. (canceled)

15. The binding agent of claim 1, further comprising a light chain constant region having the amino acid sequence set forth in SEQ ID NO: 57.

16.-19. (canceled)

20. The binding agent of claim 1, wherein the binding agent specifically binds to protein tyrosine kinase 7 (PTK7).

21. The binding agent of claim 1, wherein the VH region has the amino acid sequence set forth in SEQ ID NO: 15, the VL region has the amino acid sequence set forth in SEQ ID NO: 16, the heavy chain constant region has the amino acid sequence set forth in SEQ ID NO: 56, and the light chain constant region has the amino acid sequence set forth in SEQ ID NO: 56.

22. A pharmaceutical composition comprising the binding agent of claim 1 and a pharmaceutically acceptable carrier.

23. A nucleic acid encoding the binding agent of claim 1.

24. A vector comprising the nucleic acid of claim 23.

25. A cell line comprising the vector of claim 23.

26. A conjugate comprising:
the binding agent of claim 1,
at least one linker attached to the binding agent;
at least one drug unit, wherein each drug unit is attached to a linker, wherein the linker optionally comprises at least one polar group.

27. The conjugate of claim 26, wherein the linker is derived from a linker compound, or a stereoisomer or salt thereof, and the linker compound comprises:
a linker unit;
a stretcher group connected to the linker unit,
an optional amino acid unit; and
the at least one polar group; wherein:
the stretcher group has an attachment site to the binding agent and an attachment site to the amino acid unit (when present) or the linker subunit;
the amino acid unit (when present) has an attachment site to the stretcher unit and an attachment site to the linker unit; and
the linker unit has an attachment site to the amino acid unit (when present) or to the stretcher unit and to the at least one drug unit.

28. The conjugate of claim 27, wherein the linker unit is non-cleavable.

29. The conjugate of claim 27, wherein the polar group is attached to the amino acid unit or the stretcher group.

30. The conjugate of claim 27, wherein the linker compound comprises:
(a) the linker unit, which has from 1 to 4 attachment sites for the drug units and having one of the following structures (i) or (ii): 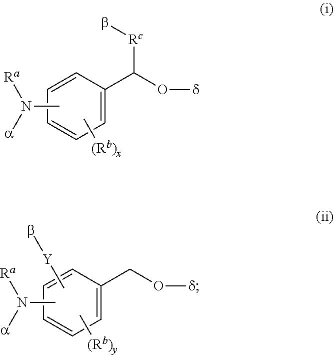
(b) the at least one polar group, each comprises a polymer unit, and
(c) the stretcher group, which has an attachment site for the binding agent;
wherein:
α— is an attachment site to an enzyme-cleavable group;
β—is an attachment site to the at least one polar group;
δ—is H, an attachment site to at least one of the drug units, or an attachment site to a linking group attached to the at least one of the drug units;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the drug units, or an attachment site to at least one of the polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl or combinations thereof.

31. The conjugate of claim 30, wherein the linker unit has one of the following structures (i-a) or (ii-a): 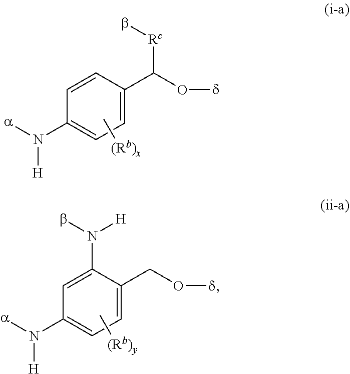
or a stereoisomer or salt thereof.

32. The conjugate of claim 30, wherein the linker unit has one of the following structures (i-b), (i-c), (i-d), (i-e) or (i-f): 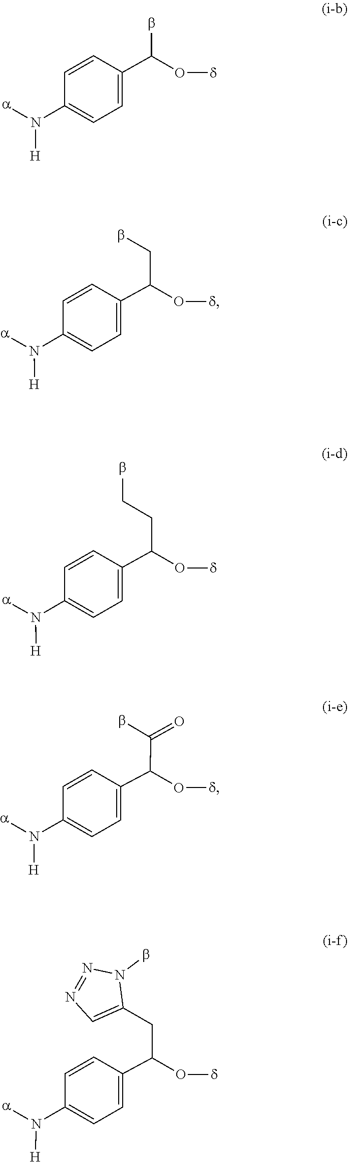
or a stereoisomer or salt thereof.

33. The conjugate of claim 30, wherein the at least one polar group comprises at least one sugar unit having the following formula:
L3-N(CH₂—(CH(XR))ₖ—X₁(X₂))₂ (X)
or a stereoisomer or salt thereof, wherein:
each X is independently selected from NH and O;
each R is independently selected from hydrogen, acetyl, a monosaccharide, a disaccharide, and a polysaccharide;
each X₁ is independently selected from CH₂ and C(O);
each X₂ is independently selected from H, OH and OR;
k is 1 to 10; and
L3 is a point of attachment to a remainder of the polar group.

34. The conjugate of claim 30, wherein the at least one polar group comprises at least one sugar unit having one of the following structures (XII) or (XIII): 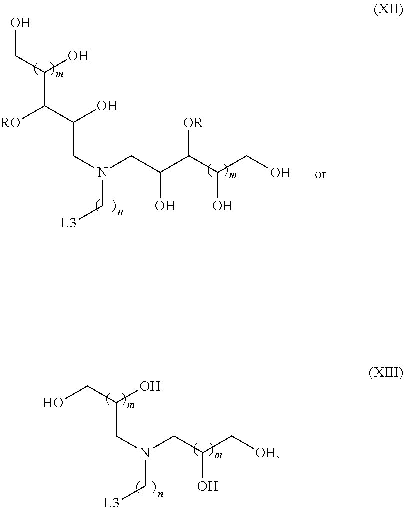
or a stereoisomer or salt thereof, wherein:
each R is independently selected from hydrogen, a monosaccharide, a disaccharide and a polysaccharide;
m is 1 to 8; and
n is 0 to 4.

35. The conjugate of claim 30, comprising a polar group having a formula of:
(a)˜R²⁰—R²¹—[O—CH₂—CH₂]ₙ₂₀—R²²—NR²⁴R²⁵ (XX)
or a stereoisomer a salt thereof, wherein:
R²⁰ is an attachment group to site β or to site R^{b}, or to the enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII), provided that R²⁴ and R²⁵ are not both H; and
n20 is 2 to 26; or
(b)˜R²⁰—R²¹—[O—CH₂—CH₂]ₙ₂₀—R²²—NR²⁴R²⁵ (XXI)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII); and the other of R²⁴ and R²⁵ is a polyethylene glycol, optionally having 1 to 24 ethylene glycol subunits; and
n20 is 2 to 26; or
(c)˜R²⁰—[—R²⁶—[R²⁹—[O—CH₂—CH₂—]ₙ₂₀R²⁹]ₙ₂₁—R²⁷—NR²⁴R²⁵]ₙ₂₇ (XXII)
or a stereoisomer or salt thereof, wherein:
R²⁰ is an attachment group to site β or to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group, where R²⁸ is a sugar unit of formula (XII) or (XIII); and polyethylene glycol, optionally having 1 to 24 ethylene glycol subunits, provided that R²⁴ and R²⁵ are not both H;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or
(d)˜R²⁰—R²¹—[—C(R^{α})H—C(O)—N(R^{N})—]ₙ₂₀—R²²—NR²⁴R²⁵ (XXIII)
or a stereoisomer or salt thereof, wherein:
R²⁰ is an attachment group to site β or to site R^{b}, or to the enzyme-cleavable group;
R²¹ is a bond or C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
R²² is C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII), provided that R²⁴ and R²⁵ are not both H; and
each n20 is independently 2 to 26, or
(e)˜R²⁰—R²¹—[—C(R^{α})H—C(O)—N(R^{N})—]ₙ₂₀—R²²—CO₂R²⁶ (XXIV)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene[O—CH₂—CH₂—]ₙ₂₀;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII), provided that R²⁴ and R²⁵ are not both H;
R²⁶ is H or C₁-C₄ alkyl; and
each n20 is independently 2 to 26,
with the proviso that at least one R^{α} or R^{N} is —R²²—NR²⁴R²⁵; or
(f)˜R²⁰—R²¹—[C(R^{α})H—C(O)—N(R^{N})—]ₙ₂₀—R²²—N—(R²³—NR²⁴R²⁵)₂ (XXV)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene-[O—CH₂—CH₂—]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)-polyhydroxyl group; substituted C₁-C₈ alkyl; substituted —C(O)—C₁-C₈ alkyl; a chelator; and —C(O)—R²⁸, where R²⁸ is a sugar unit of formula (XII) or (XIII), provided that R²⁴ and R²⁵ are not both H; and
each n20 is independently 2 to 26.

36. The conjugate of claim 35, wherein R²⁴ and R²⁵ are each independently selected from H and a polyhydroxyl group, provided that R²⁴ and R²⁵ are not both H.

37. The conjugate of claim 35, wherein the polyhydroxyl group is a linear monosaccharide, optionally selected from a C₆ or C₅ sugar, a sugar acid and an amino sugar.

38. The conjugate of claim 37, wherein:
the C₆ or C₅ sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

39. The conjugate of claim 35, comprising a polar group selected from the following, or a stereoisomer or salt thereof: 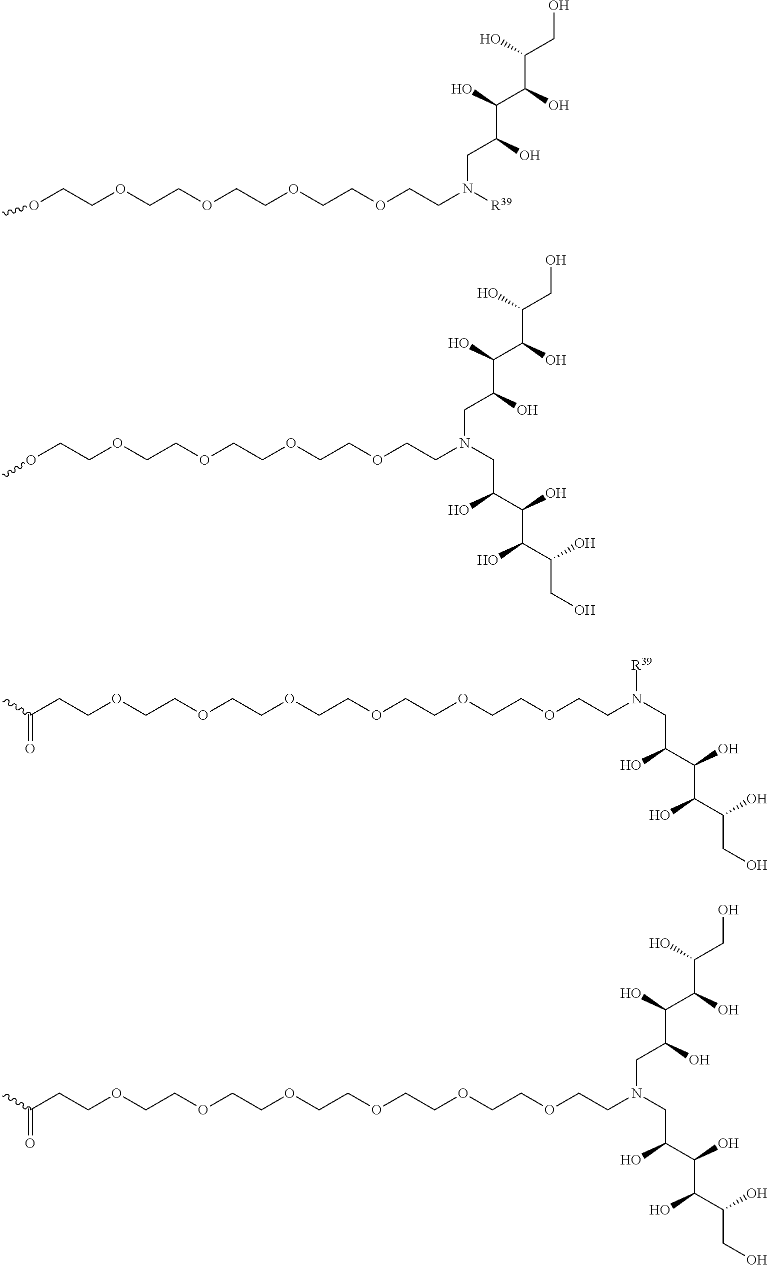 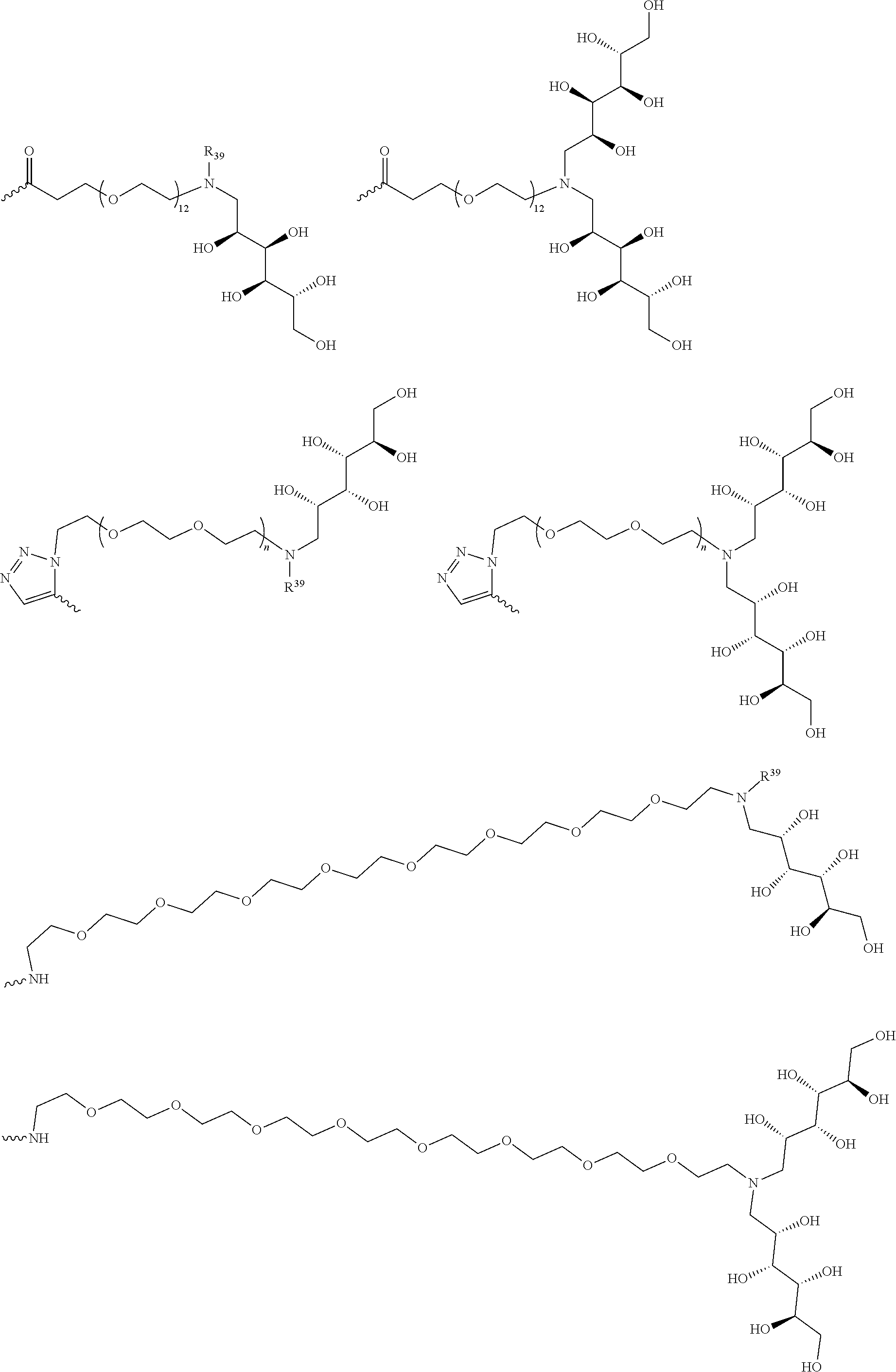 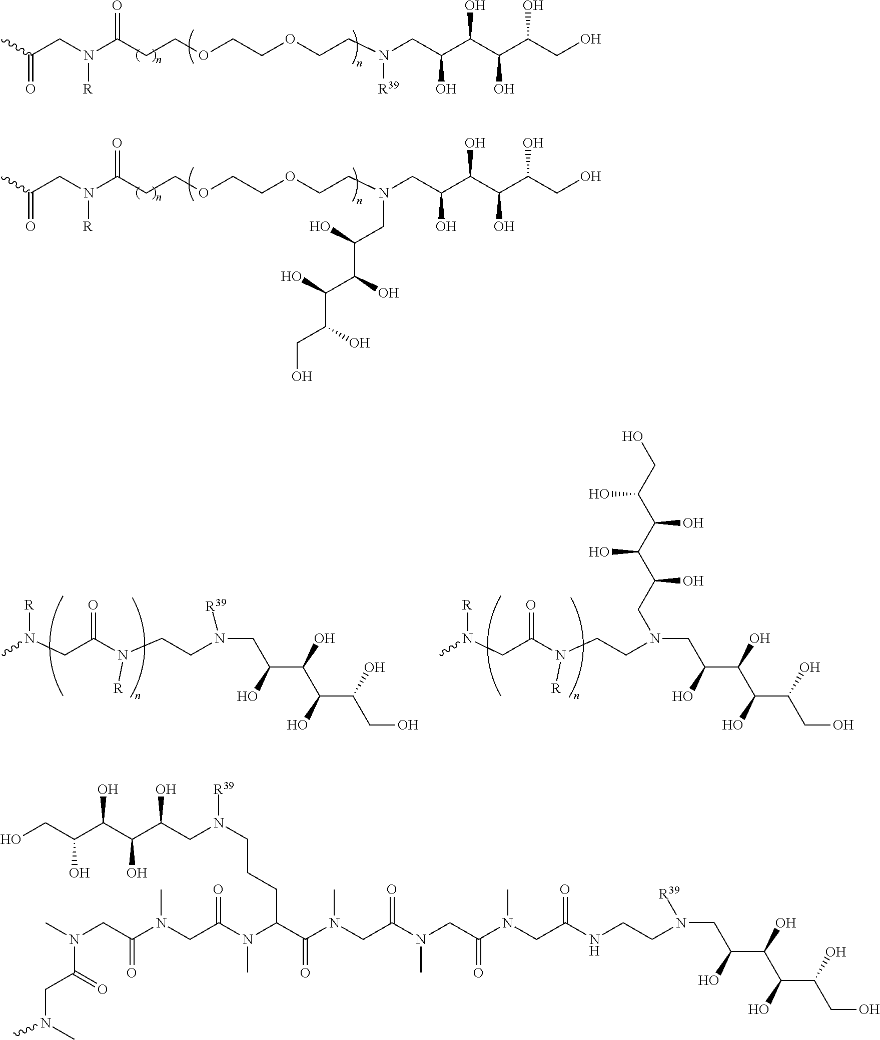 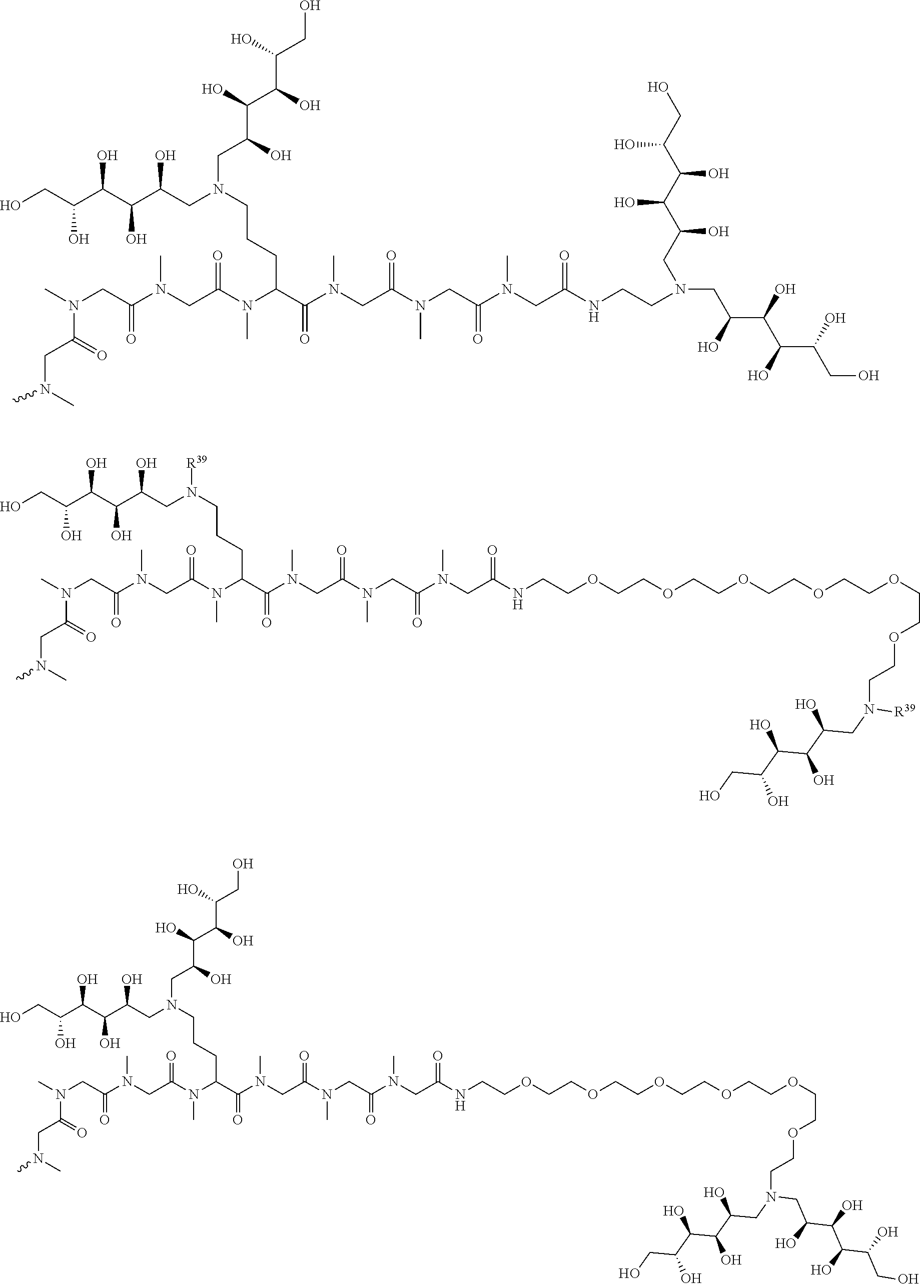 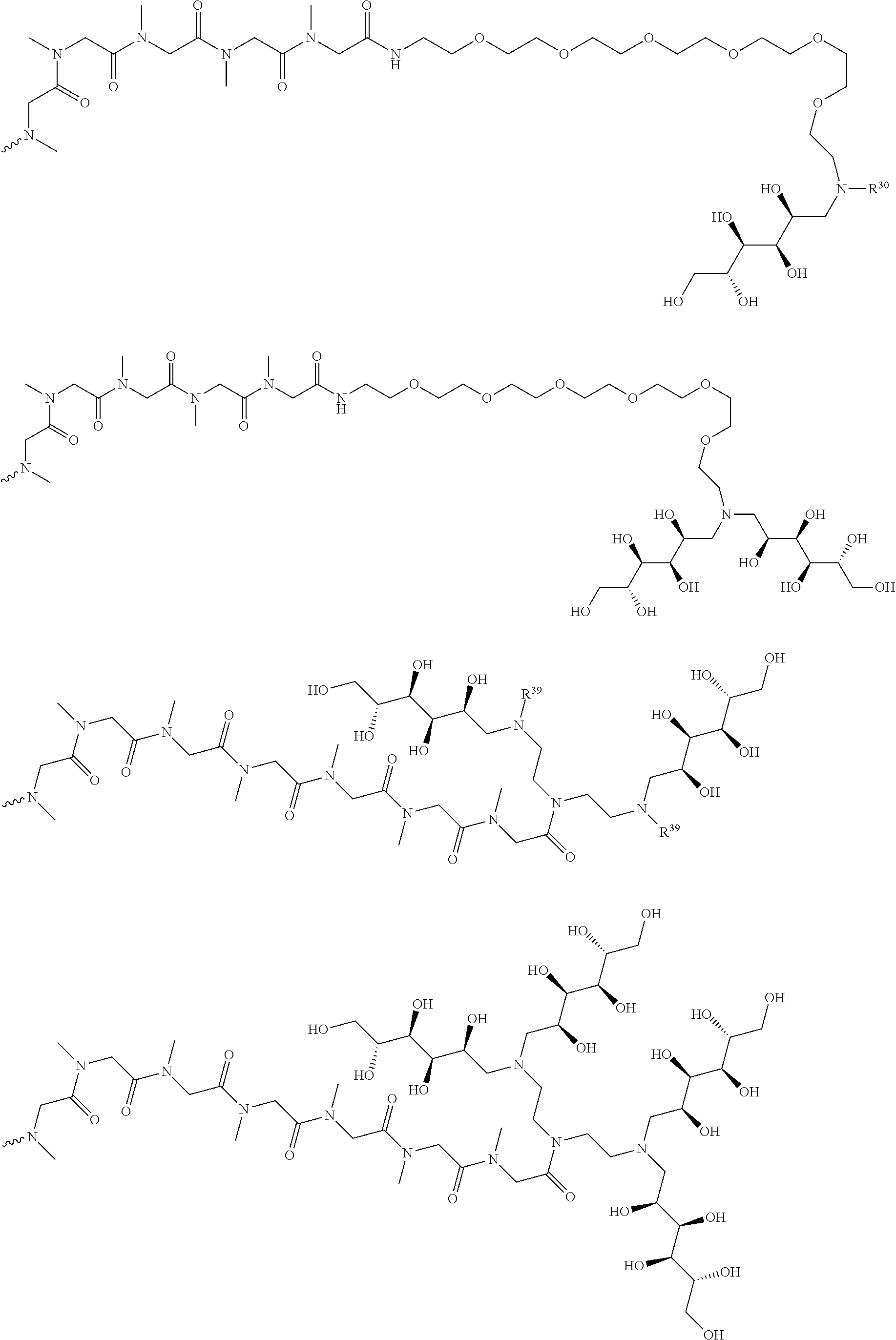 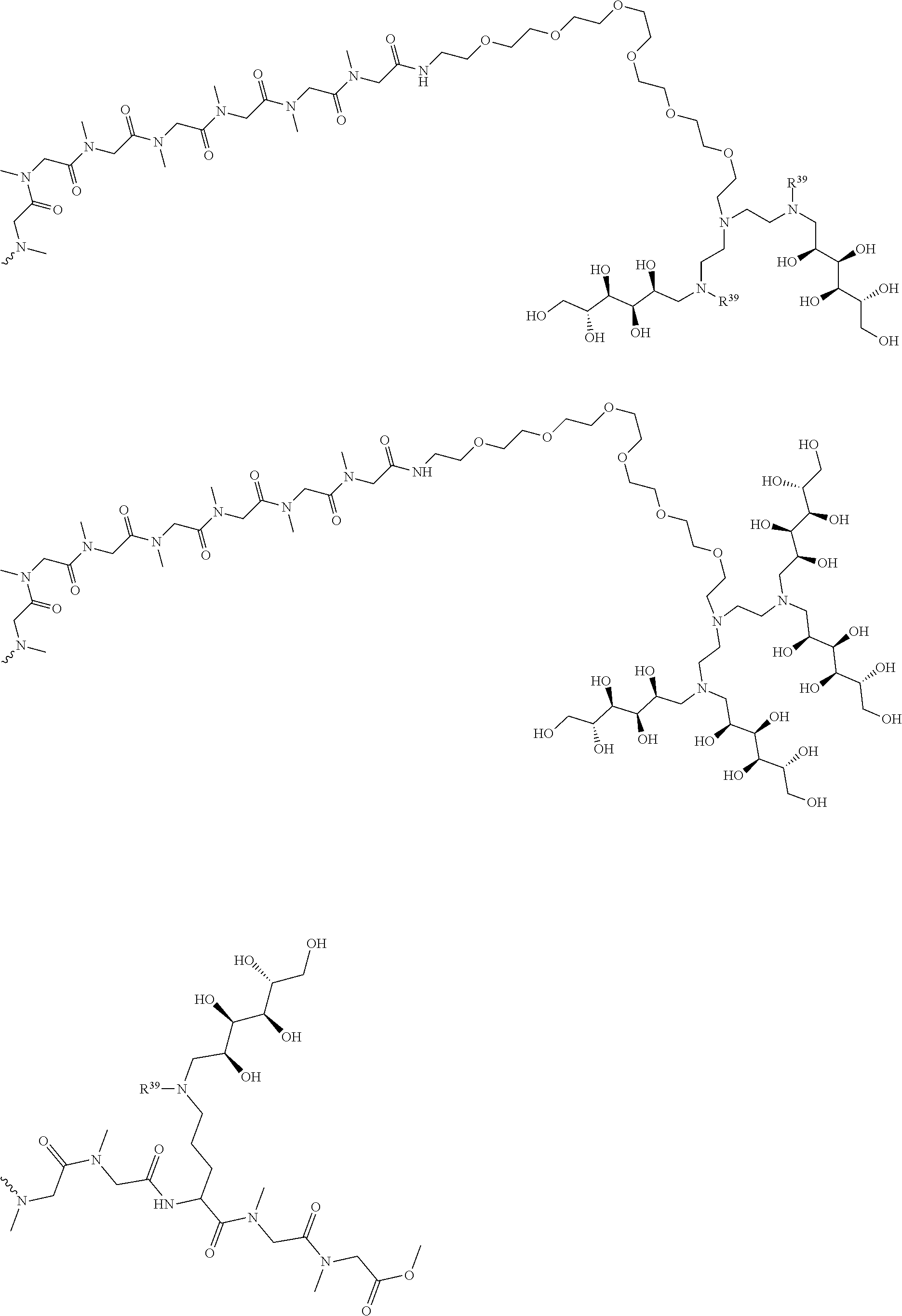 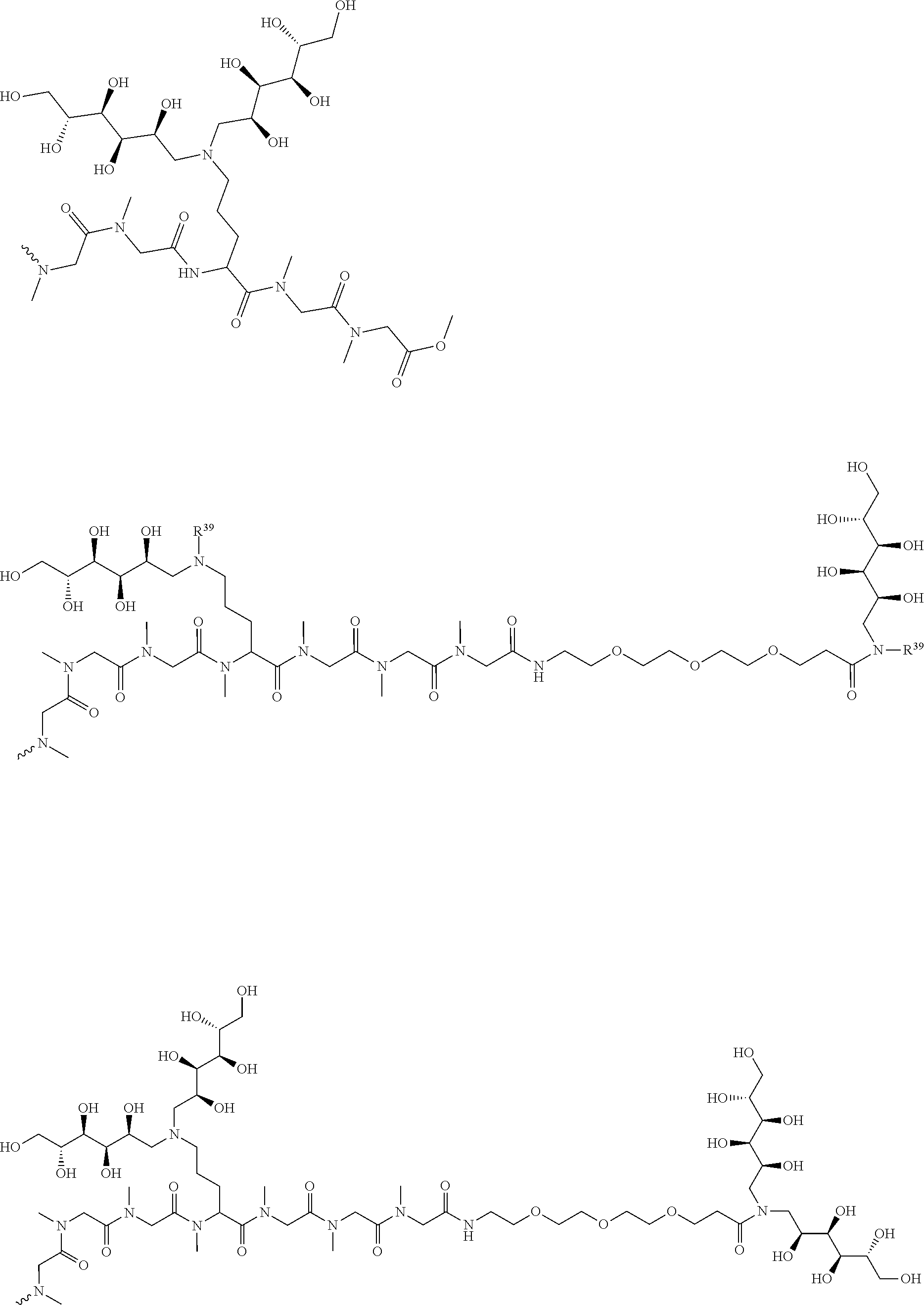 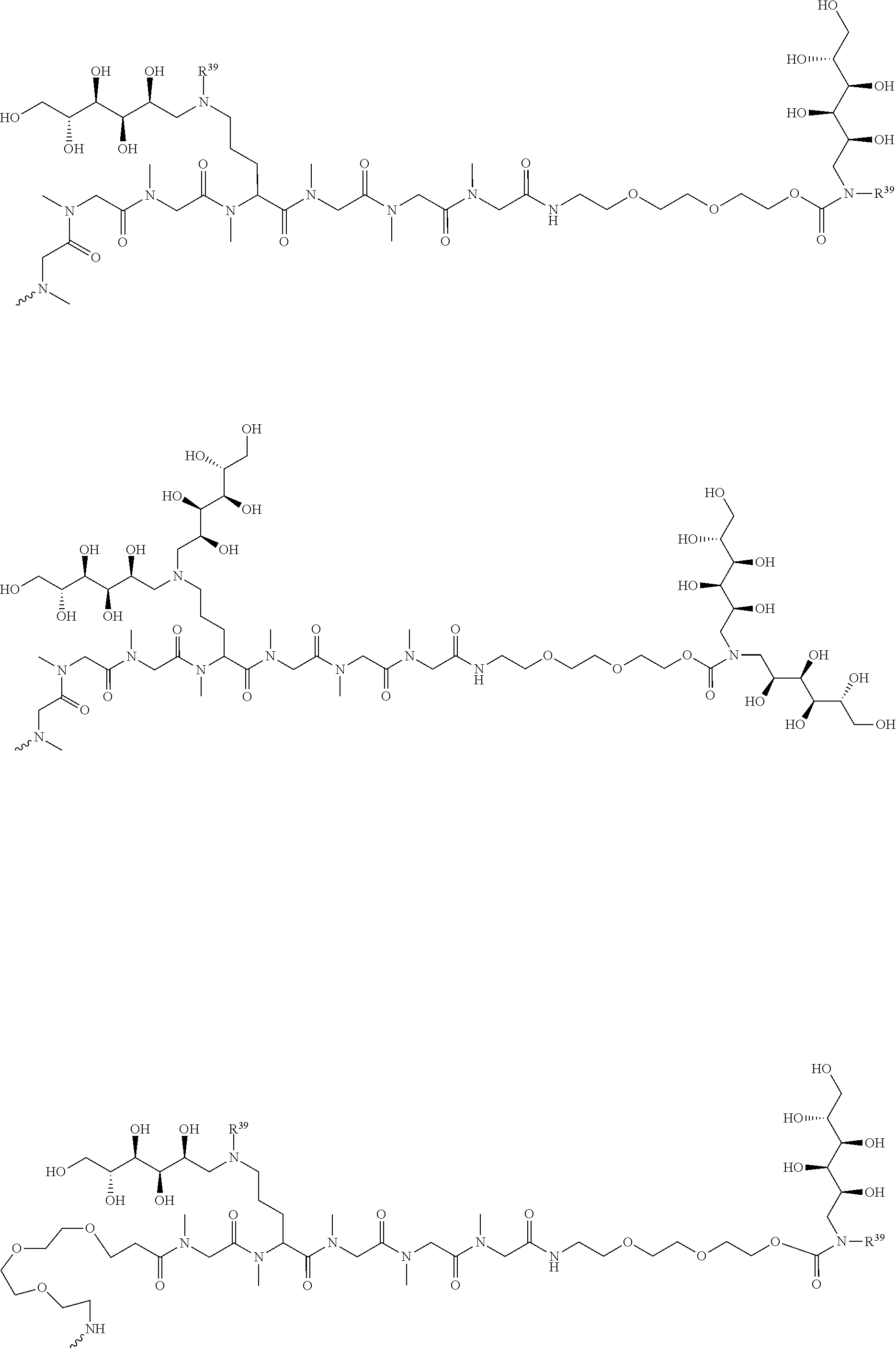 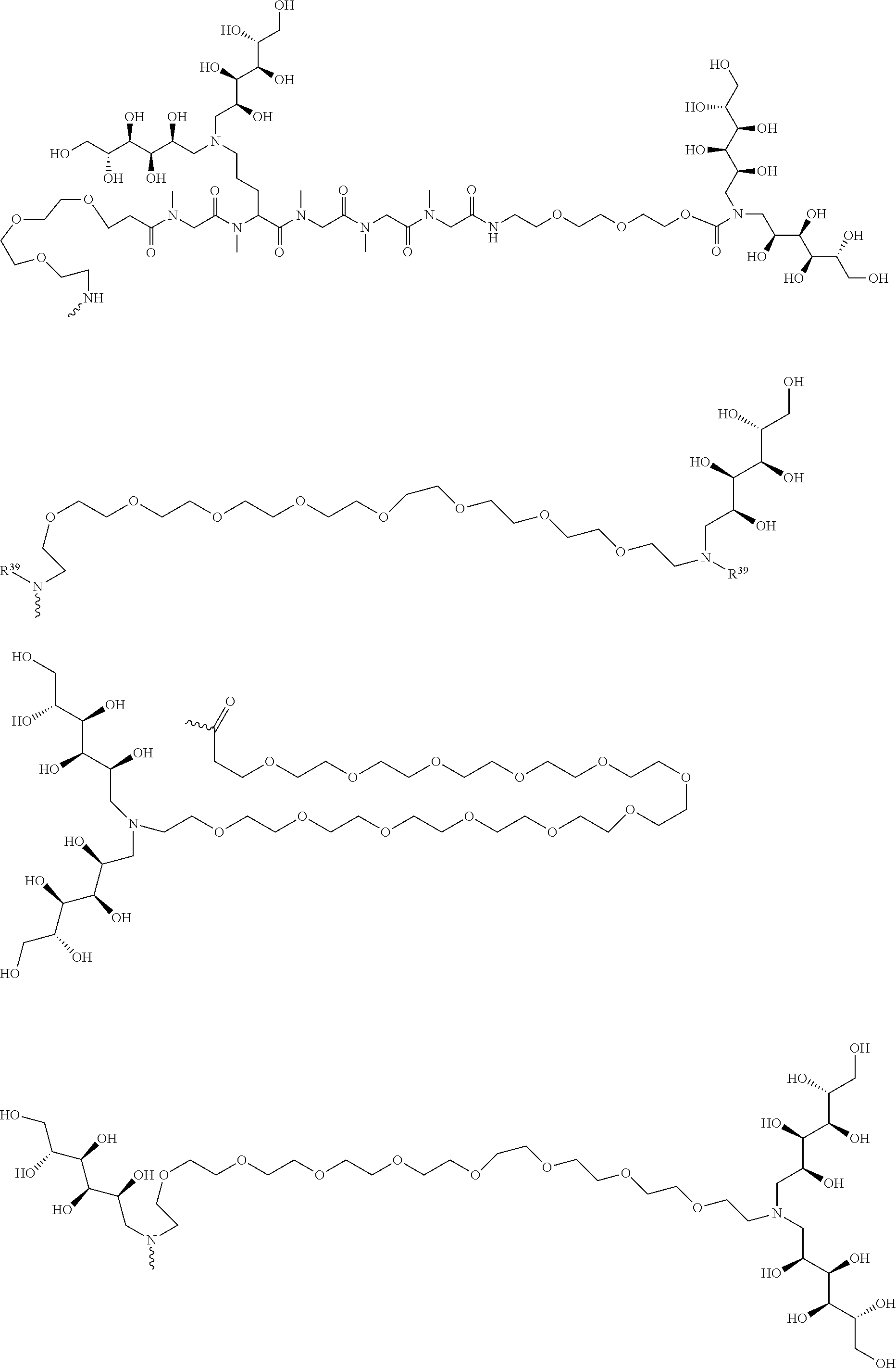 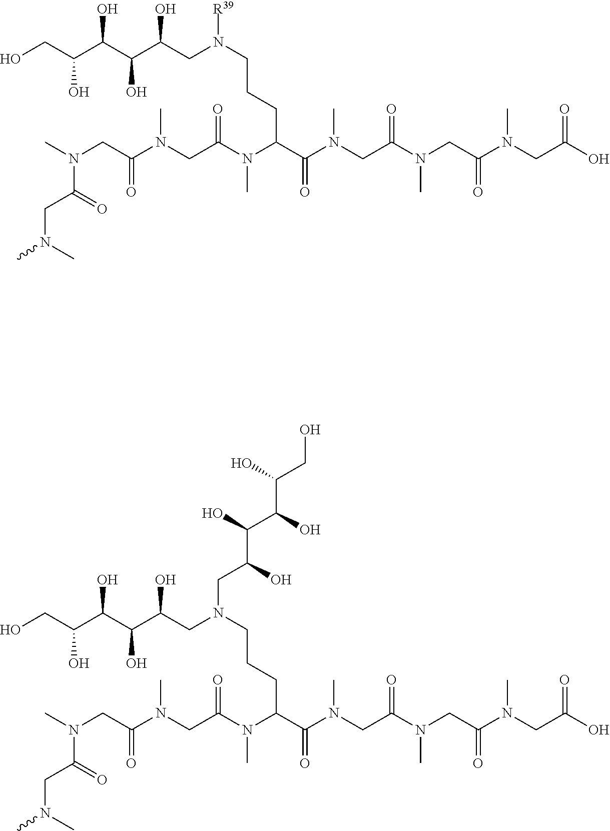
wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment to site β or to site R^{b}, or to the enzyme-cleavable group.

40. The conjugate of claim 30, wherein the attachment site β is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

41. The conjugate of claim 30, comprising a polar group having a formula selected from the following:
(a)˜R²⁰—R²¹—[O—CH₂—CH₂]ₙ₂₀—R²²—R³⁰ (XXX)
or a stereoisomer or salt thereof, wherein:
R²⁰ is an attachment group to site β to site R^{b}, or to the enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH— (cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;
(b)˜R²⁰—R²¹—[O—CH₂—CH₂]ₙ₂₀—R²²—NH—C(O)—R³¹ (XXXI)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;
(c)˜R²⁰—R²¹—[O—CH₂—CH₂]ₙ₂₀—R²²—C(O)NH—R³¹ (XXXII)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26;
(d)˜R²⁰—R²¹—[O—CH₂—CH₂]ₙ₂₀—R²²—C(O)NR¹—R²²—NR²⁴R²⁵ (XXXIII)
or a stereoisomer or salt thereof, wherein:
R²⁰ is an attachment group to site β or to site R^{b}, or to the enzyme-cleavable group;
R³¹ is H or R²²—NR²⁴R²⁵;
R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)-polyhydroxyl group, provided that R²⁴ and R²⁵ are not both H; and
n20 is 2 to 26;
(e)˜R²⁰—R²¹—[O—CH₂—CH₂]ₙ₂₀—R²²—N(R³³—R³¹)₂ (XXXIV)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;
(f)˜R²⁰—(R²¹—[CH₂—CH(OR³⁴)—CH₂—O]ₙ₂₀—R³⁶)ₙ₂₅ (XXXV)
or a stereoisomer or salt thereof, wherein:
R²⁰ is an attachment group to site β or to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})—C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group, provided that both R⁴⁴ and R⁴⁵ are not H;
each n20 is independently 1 to 26; and
n25 is 1 or 2;
(g)˜R²⁰—R²¹—[[CH₂—CH₂—O]ₙ₂₀—R²²—[CH₂—[CH(OH)]ₙ₂₃—CH₂—O]ₙ₂₁]ₙ₂₂—R²³—NR²⁴—R²⁵ (XXXVI)
or a stereoisomer or salt thereof, wherein:
R²⁰ is an attachment group to site β or to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; and substituted —C(O)-polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2;
(h)—R²⁰—(R₂₁—[O—CH₂—CH₂]ₙ₂₀—R²²—N(R^{N})—CO₂—[CH₂—CH(OR³⁴)—CH₂—O]ₙ₂₁—R³⁶)ₙ₂₅ (XXXVII)
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁-C₆alkylene-NR²⁴R²⁵, C₁-C₆alkylene-C(O)NR²⁴R²⁵ or C₁-C₆alkylene-C₀₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; and substituted —C(O)-polyhydroxyl group; provided that both R⁴⁴ and R⁴⁵ are not H;
n20 is 2 to 26;
n21 is 1 to 26; and
n25 is 1 or 2;
(i)˜R²⁰—(R²¹—[N(R^{N})—C(O)—[O—CH₂—CH(OH)—CH₂]ₙ₂₀]ₙ₂₁—R²²—NR²⁴R²⁵)ₙ₂₅ (XXXVIII)
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; and
substituted polyhydroxyl group, provided that R²⁴ and R²⁵ are not both H;
n20 is 2 to 26;
n21 is 1 to 4; and
n25 is 1, 2 or 3;
(j)˜R²⁰—(R²¹—[C(R^{α})H—C(O)—N(R^{N})]ₙ₂₀—R²²—[CH₂—CH₂—O]ₙ₂₀—NR²⁴R²⁵)ₙ₂₅ (XXXIX)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; —C(O)—R²⁸, wherein R²⁸ is a sugar unit of formula (XII) or (XIII), provided that R²⁴ and R²⁵ are not both H;
each n20 is independently 0 to 26, with the proviso that at least one n20 is 2 to 26; and
n25 is 1 or 2; or
(k)˜R²⁰—R²¹—[C(R^{α})H—C(O)—N(R^{N})]ₙ₂₀—R²²—[CH₂—CH₂—O]ₙ₂₀—NR²⁴R²⁵
R²¹—[C(R^{α})H—C(O)—N(R^{N})]ₙ₂₁—R²²—[CH₂—CH₂—O]ₙ₂₁—R²³—CO₂—R²⁶ (XXXVX)
or a stereoisomer or salt thereof, wherein:
R²⁰ is an attachment group to site β or to site R^{b}, or to the enzyme-cleavable group;
R²¹, R²² and R²³ are each, independently, a bond, C₁-C₃ alkylene, —C₁-C₃alkylene-[O—CH₂—CH₂—]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a sugar unit of formula (XII) or (XIII), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

42. The conjugate of claim 30, comprising a polar group having a formula selected from the following, or a stereoisomer or salt thereof:
˜R²⁰—R²¹—[O—CH₂—CH₂]ₙ₂₀—R²²—NH—C(O)—R³¹ (XXXI),
—R²⁰—R²¹—[O—CH₂—CH₂]ₙ₂₀—R²²—C(O)NH—R³¹ (XXXII), and
˜R²⁰—R²¹—[O—CH₂—CH₂]ₙ₂₀—R²²—N—(R³³—R³¹)₂ (XXXIII);
wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³³ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

43. The conjugate of claim 41, wherein the attachment to site β to site Rb, or to the enzyme-cleavable group is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

44. The conjugate of claim 30, comprising a polar group having a formula:
˜R²⁰—(R⁴³—R⁴¹—[O—CH₂—CH₂]ₙ₄₀—R⁴²—R⁴³—(NR⁴⁴R⁴⁵)ₙ₄₁)ₙ₄₂ (XL)
or a stereoisomer or salt thereof, wherein:
R²⁰ is an attachment group to site β or to site R^{b}, or to the enzyme-cleavable group;
R⁴¹ and R⁴² are each, independently, a bond or C₁-C₆ alkylene;
each R⁴³ is, independently, selected from a bond, C₁-C₁₂ alkylene, —OC₁-C₁₂ alkylene, —C(═O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group, and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26, provided that R⁴⁴ and R⁴⁵ are not both H;
n40 is 2 to 26;
n41 is 1 to 6; and
n42 is 1 to 6.

45. The conjugate of claim 30, comprising a polar group having a formula:
˜R²⁰—(R⁴¹—[O—CH₂—CH₂]ₙ₄₀—R⁴²—R⁴³—(NR⁴⁴R⁴⁵)ₙ₄₁)ₙ₄₂ (XLI)
or a stereoisomer or salt thereof, wherein:
R²⁰ is an attachment group to site β or to site R^{b}, or to the enzyme-cleavable group;
R⁴¹ and R⁴² are each, independently, a bond or C₁-C₆ alkylene;
R⁴³ is selected from a bond, C₁-C₁₂ alkylene, —OC₁-C₁₂ alkylene, —C(═O)—, —NR^{a}—C₁-C₁₂ alkylene, —C₁-C₁₂ alkylene-N′R^{a}—, —C(O)—C₁-C₁₂ alkylene, —C₁-C₁₂ alkylene-C(O)—, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, C(O)—NR^{a}—C₁-C₁₂ alkylene, -heteroarylene, heteroaryl-C₁-C₁₂ alkylene, heteroaryl-C₁-C₁₂ alkylene-C(O)—, and —C(O)NR⁴⁶R⁴⁷, wherein each alkylene is optionally substituted with hydroxyl, SO₃H and/or oxo, R^{a} is H, C₁-C₆ alkyl, a polyhydroxyl group, or a substituted polyhydroxyl group and one of R⁴⁶ and R⁴⁷ is H or C₁-C₁₂ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 26;
n41 is 1 to 6; and
n42 is 1 to 6.

46. The conjugate of claim 30, comprising a polar group having a formula:
˜R²⁰—(R⁴¹—[O—CH₂—CH₂]ₙ₄₀—R⁴²—R⁴³—(NR⁴⁴R⁴⁵)ₙ₄₁)ₙ₄₂ (XLII)
or a stereoisomer or salt thereof, wherein:
R²⁰ is an attachment group to site β or to site R^{b}, or to the enzyme-cleavable group;
R⁴¹ and R⁴² are each, independently, a bond or C₁-C₃ alkylene;
R⁴³ is selected from a bond, C₁-C₆ alkylene, —OC₁-C₁₂ alkylene, —C(═O)—, —NR^{a}—C₁-C₁₂ alkylene, —C₁-C₆ alkylene-NR^{a}—, —C(O)—C₁-C₆ alkylene, —C₁-C₆ alkylene-C(O)—, —NR^{a}—C₁-C₆ alkylene-C(O)—, —C(O)—C₁-C₆ alkylene-NR^{a}—, —NR^{a}—C(O)—NR^{a}—, —NR^{a}—C(O)—, —NR^{a}—C(O)—C₁-C₆ alkylene, —C(O)—NR^{a}—C₁-C₁₂ alkylene, -heteroarylene, heteroaryl-C₁-C₆ alkylene, heteroaryl-C₁-C₆ alkylene-C(O)—, and —C(O)NR⁴⁶R⁴⁷, wherein each alkylene is optionally substituted with hydroxyl, SO₃H, and/or oxo, R^{a} is H, C₁-C₆ alkyl, a polyhydroxyl group, or a substituted polyhydroxyl group and one of R⁴⁶ and R⁴⁷ is H or C₁-C₆ alkylene and the other is C₁-C₁₂ alkylene, wherein one of the C₁-C₂ alkylenes is bound to NR⁴⁴R⁴⁵ at the nitrogen atom;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
n40 is 1 to 16;
n41 is 1 to 4; and
n42 is 1 to 4.

47. The conjugate of claim 35, wherein R²⁰ is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

48. The conjugate of claim 35, wherein R²⁰ comprises one of the following structures: 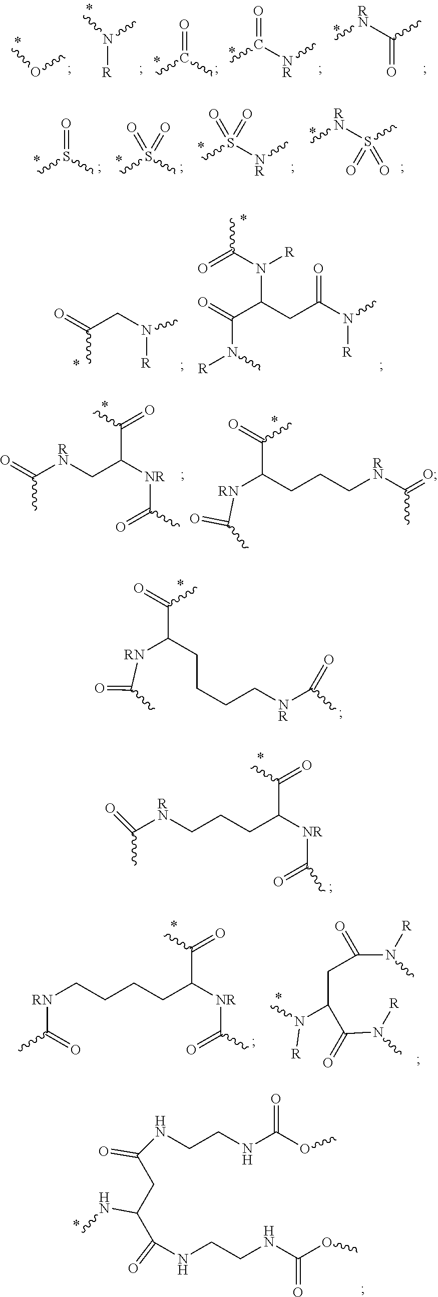 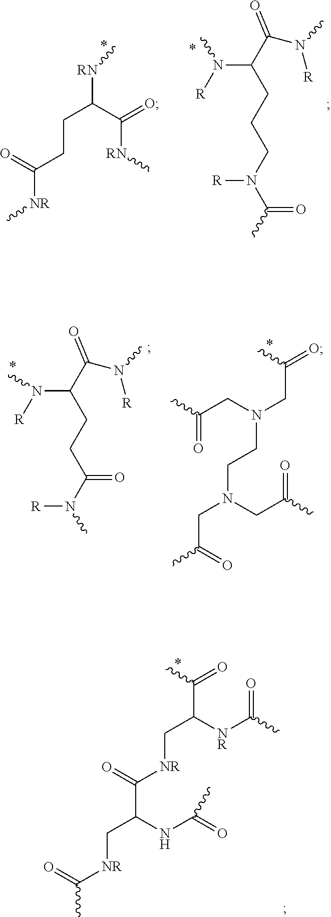 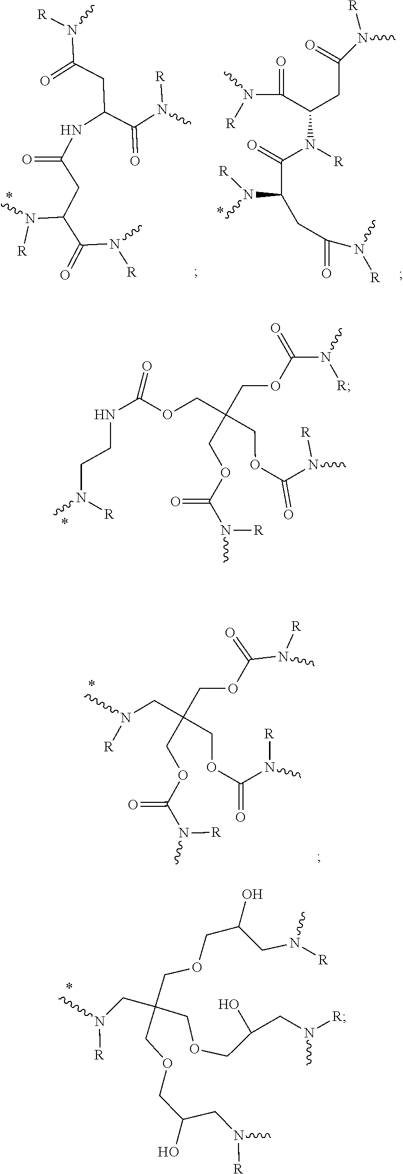 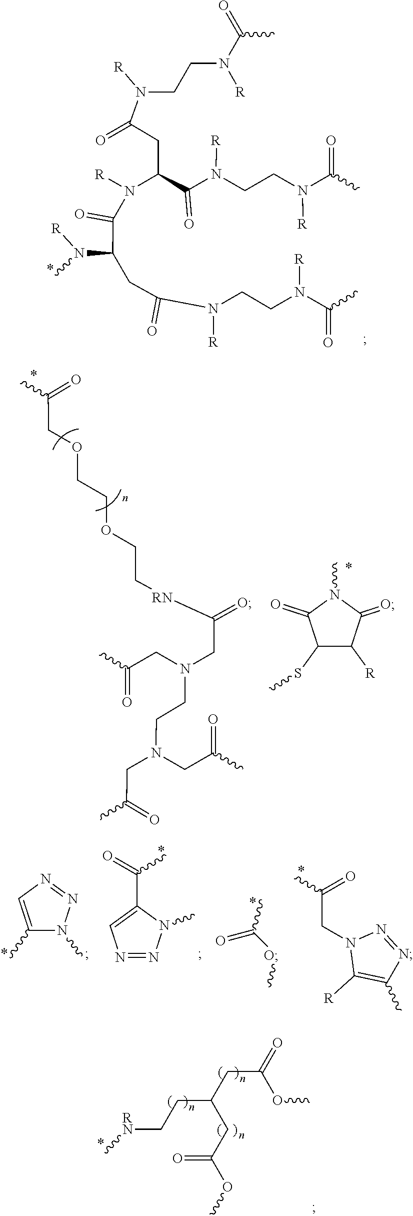 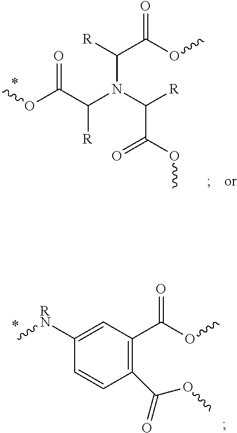
or a stereoisomer thereof, wherein R is H, C₁-C₆ alkyl or polyhydroxyl group, n is 0 to 12, the ( [symbol] *) indicates an attachment to site β or to site R^{b}, or to the enzyme-cleavable group, and the ( [symbol] ) indicates an attachment site to a remainder portion of the polar group.

49. The compound of claim 35, wherein R²⁰ has one of the following structures: 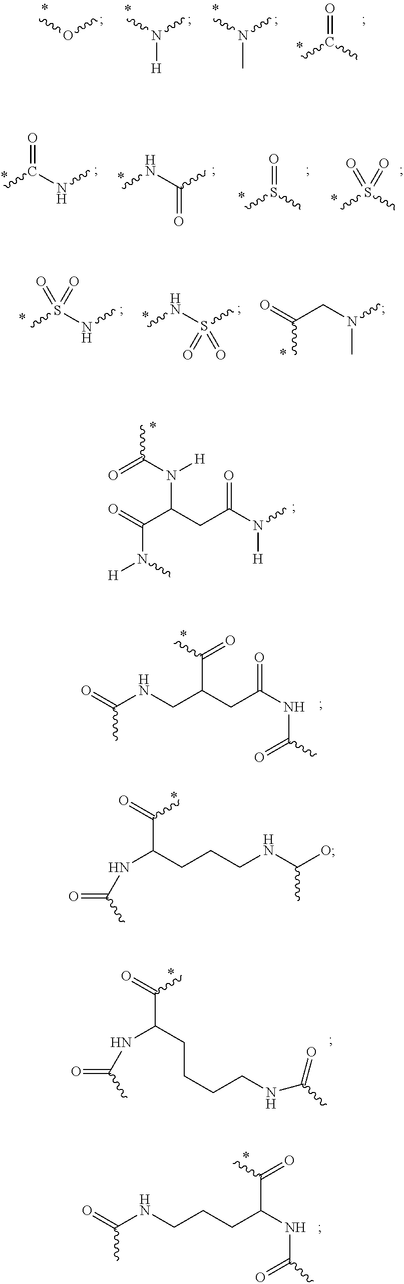 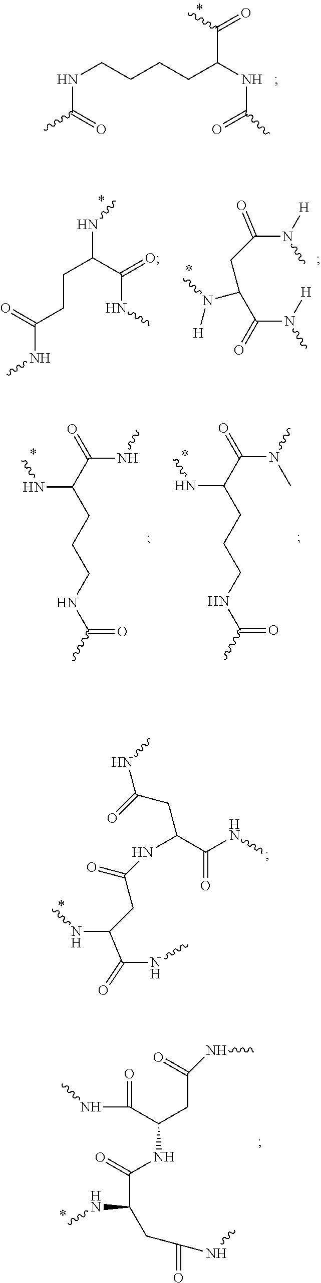 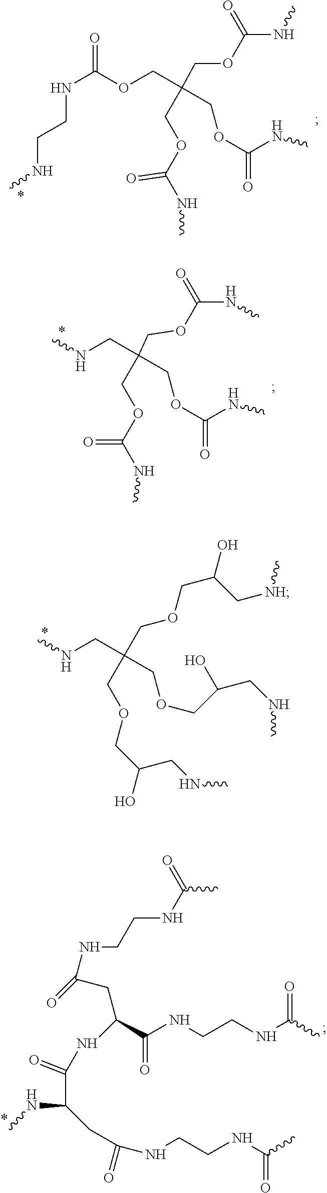 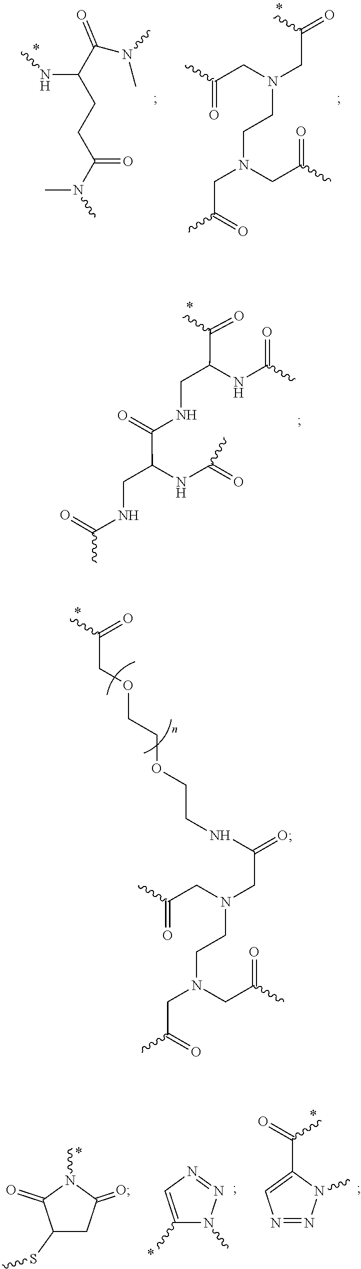 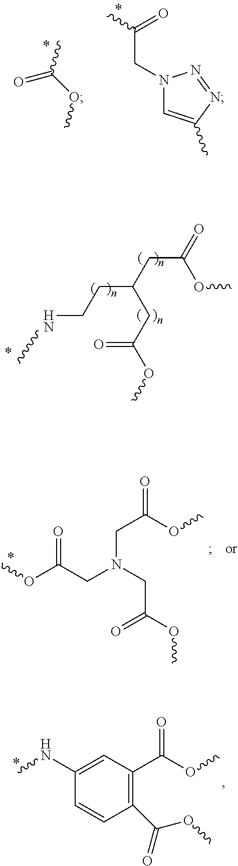
or a stereoisomer thereof, wherein n=0 to 12, the ( [symbol] *) indicates an attachment to site β or to site R^{b}, or to the enzyme-cleavable group, and the ( [symbol] ) indicates an attachment site to a remainder portion of the polar group.

50. The conjugate of claim 44, wherein R⁴³—(NR⁴⁴R⁴⁵)n⁴¹ has one of the following structures: 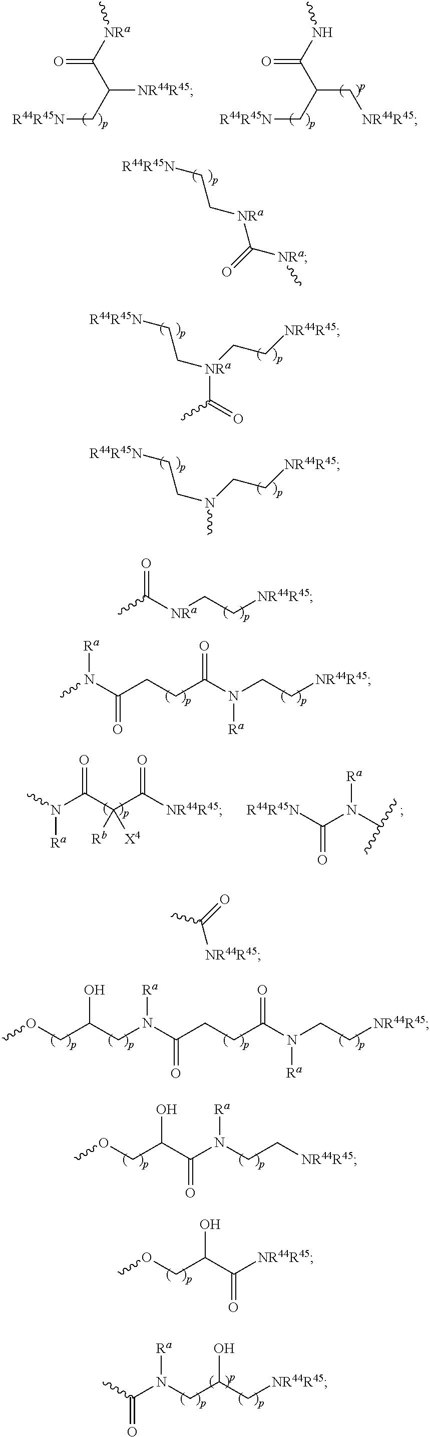 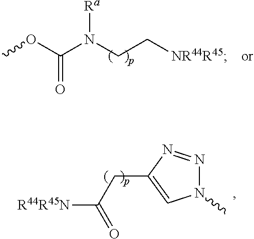
or a stereoisomer thereof, wherein R^{a} is H, C₁-C₆ alkyl, a polyhydroxyl group, or a substituted polyhydroxyl group; p is an integer from 1 to 6, and the ( [symbol] ) indicates the attachment site of R⁴³ to the remainder of the polar group.

51. The conjugate of claim 44, wherein R⁴³—(NR⁴⁴R⁴⁵)n⁴¹ has one of the following structures: 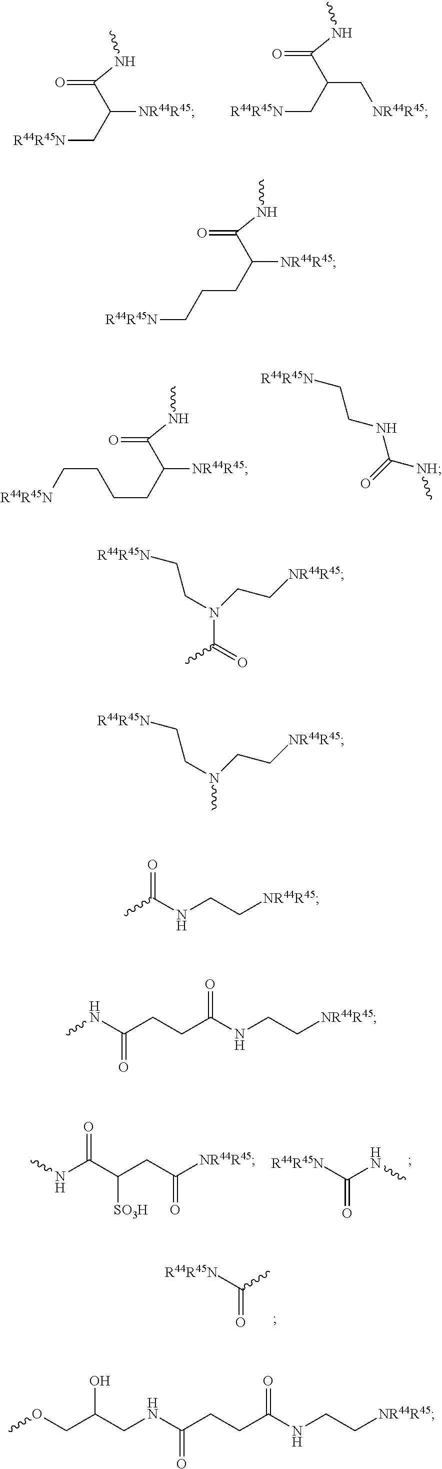 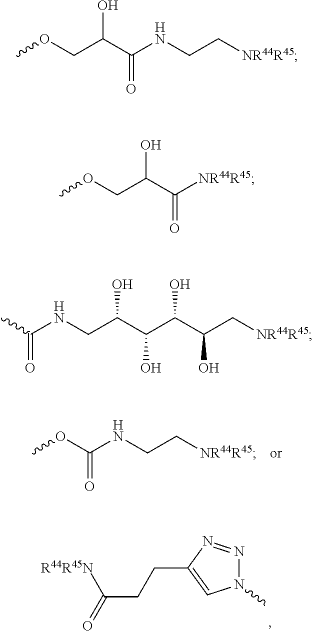
or a stereoisomer thereof, wherein the ( [symbol] ) indicates the attachment site of R⁴³ to the remainder of the polar group.

52. The conjugate of claim 44, wherein —NR⁴⁴R⁴⁵ has one of the following structures: 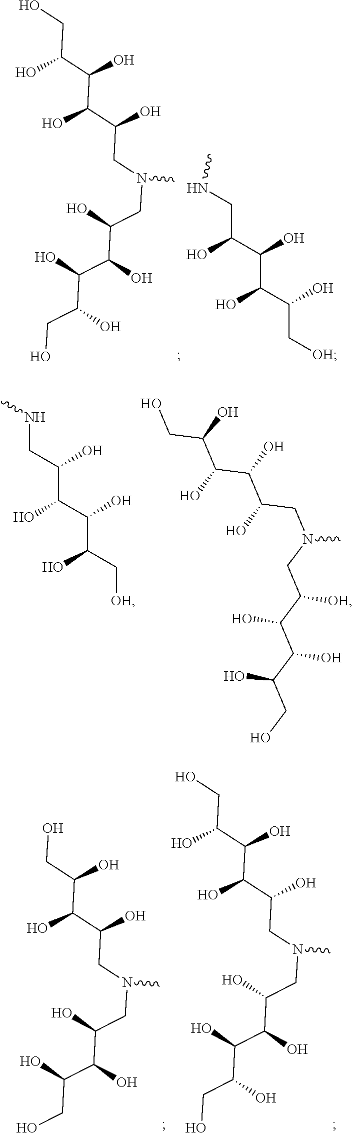 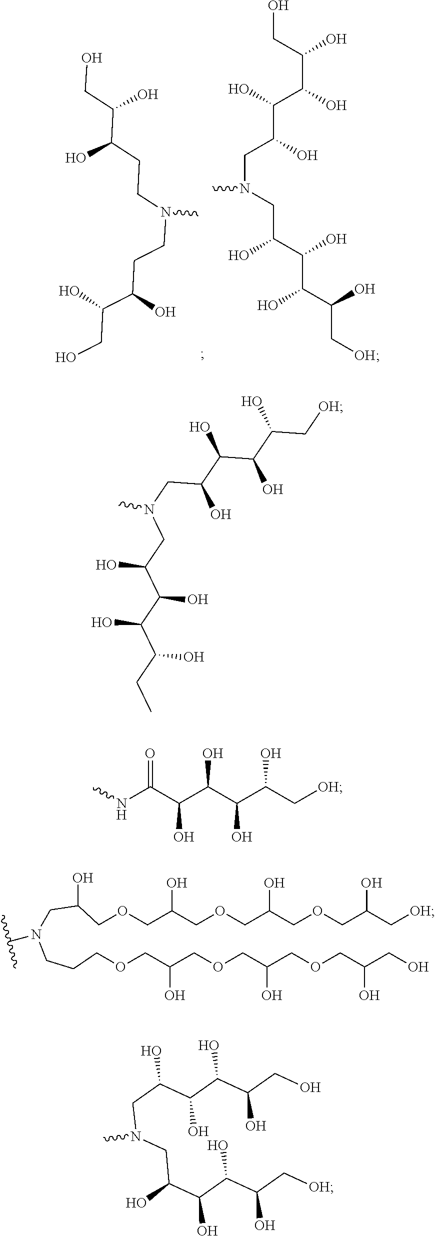 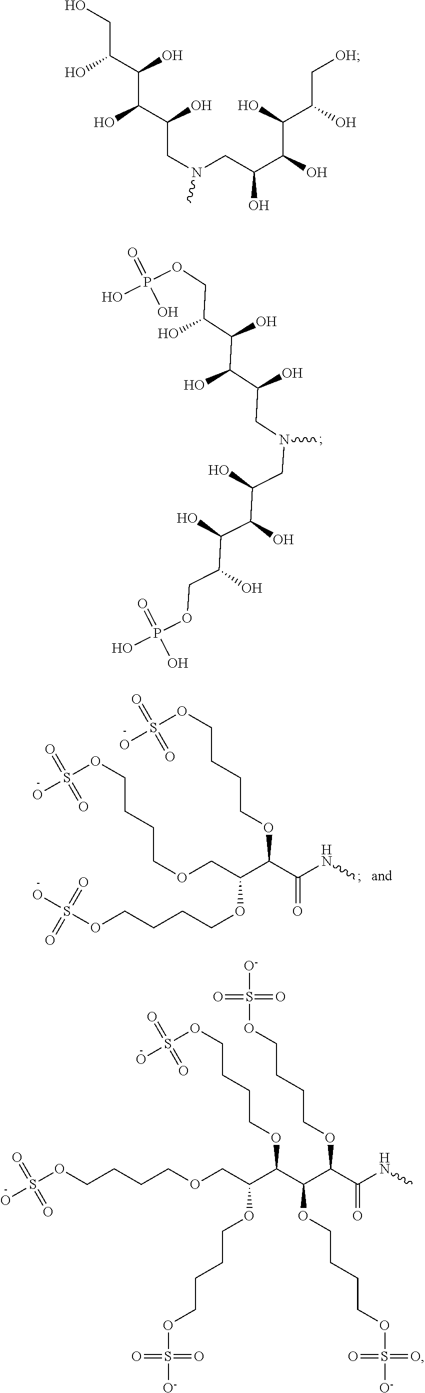
or a stereoisomer thereof, wherein the ( [symbol] ) indicates the attachment site of —NR⁴⁴R⁴⁵ to the remainder of the polar group.

53. The conjugate of claim 30, comprising a polar group having one of the following structures prior to attachment to the linker unit: 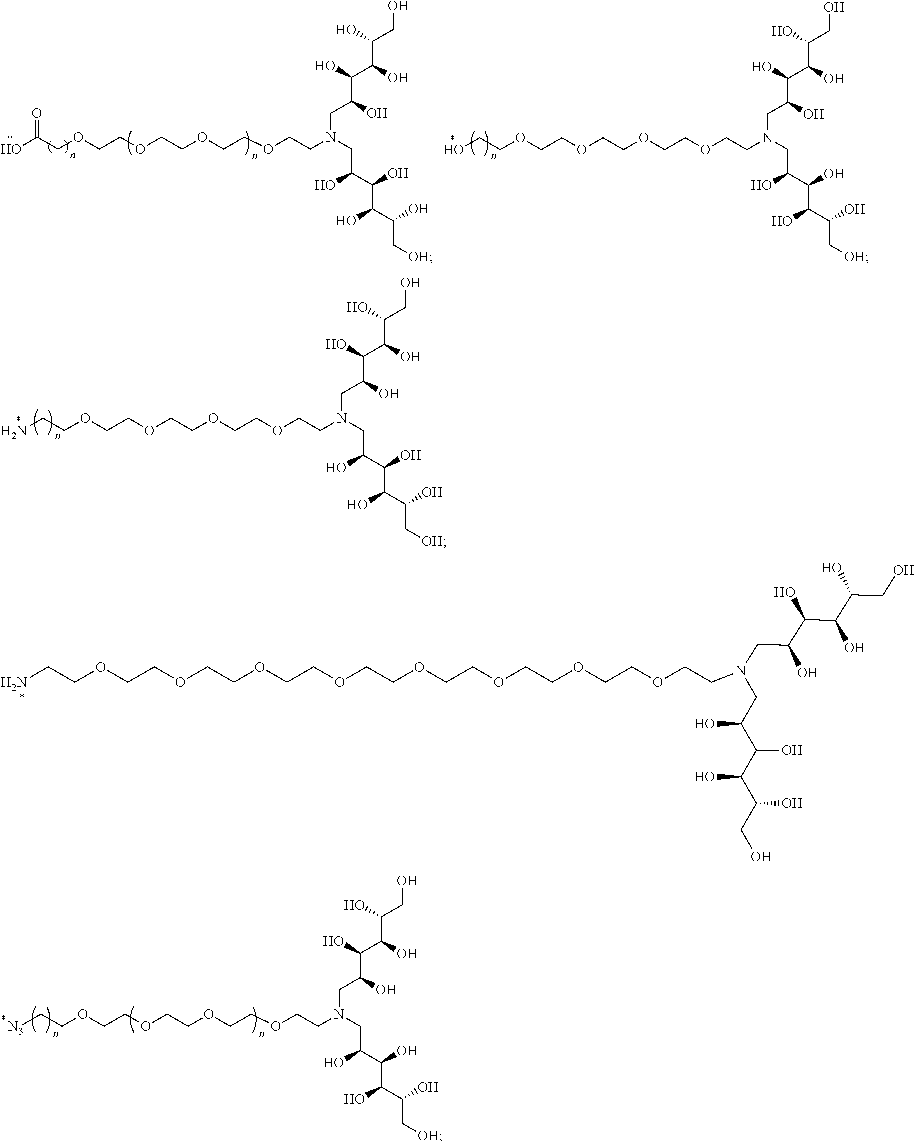 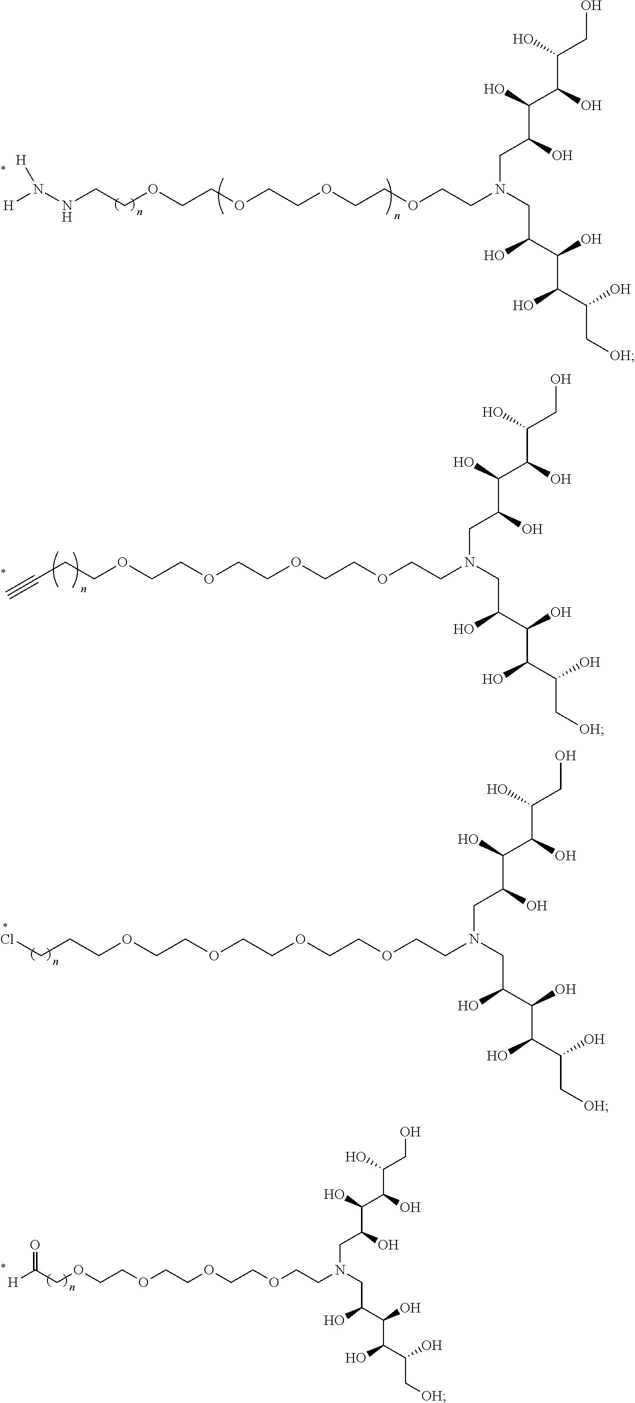 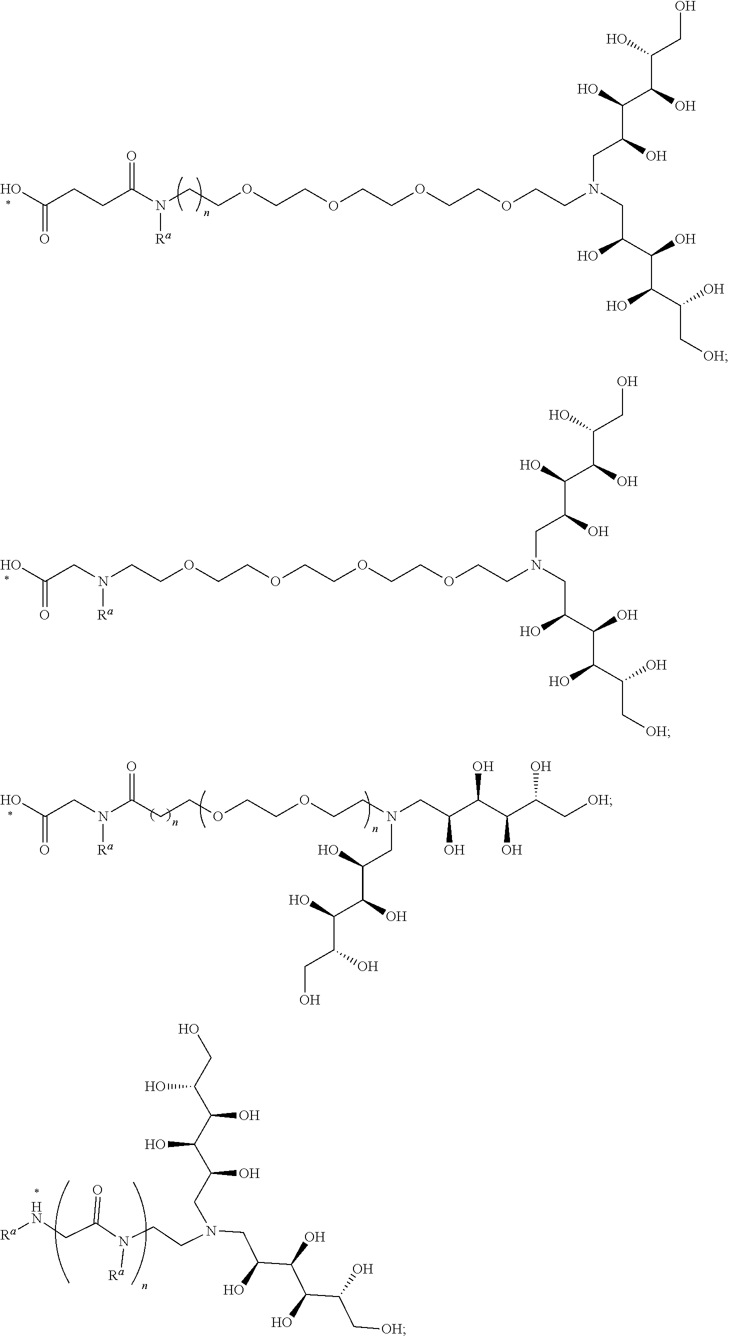 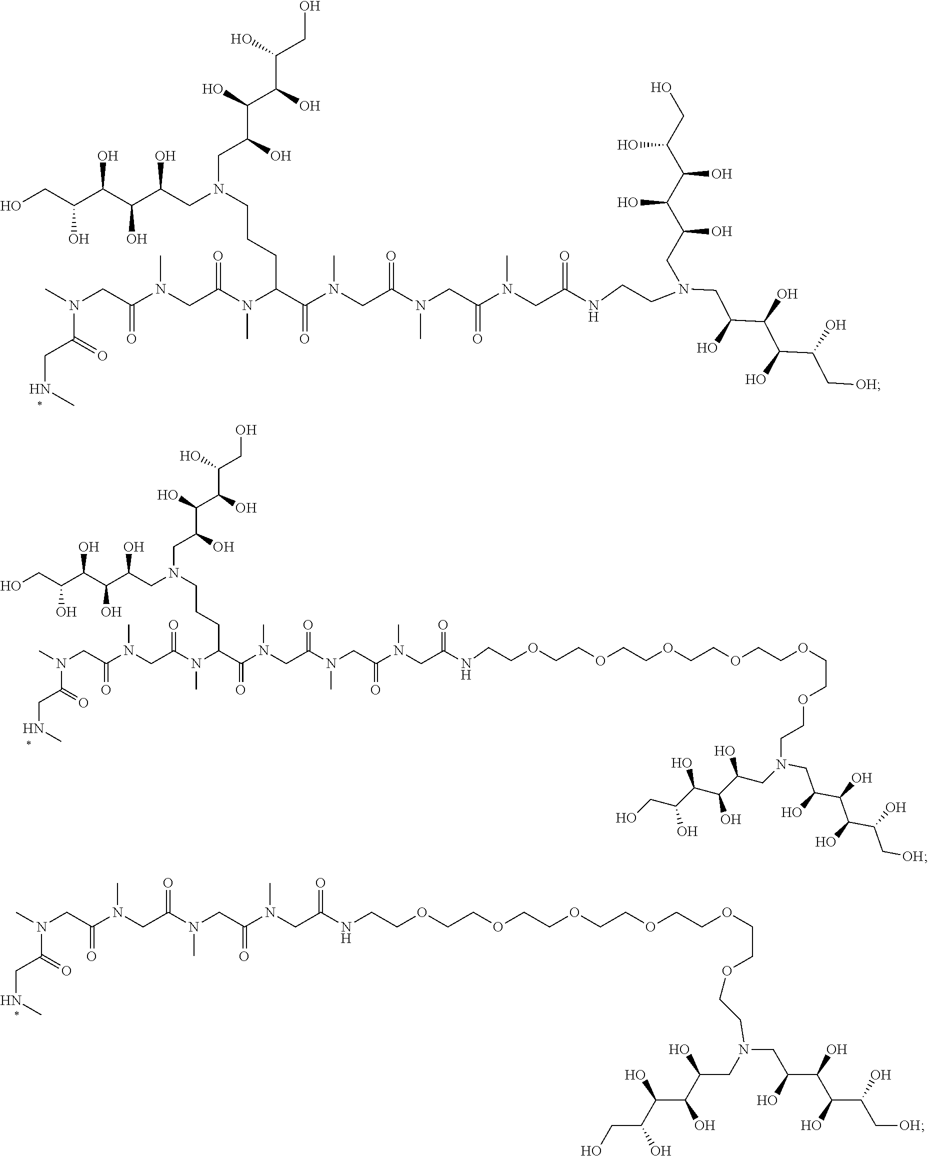 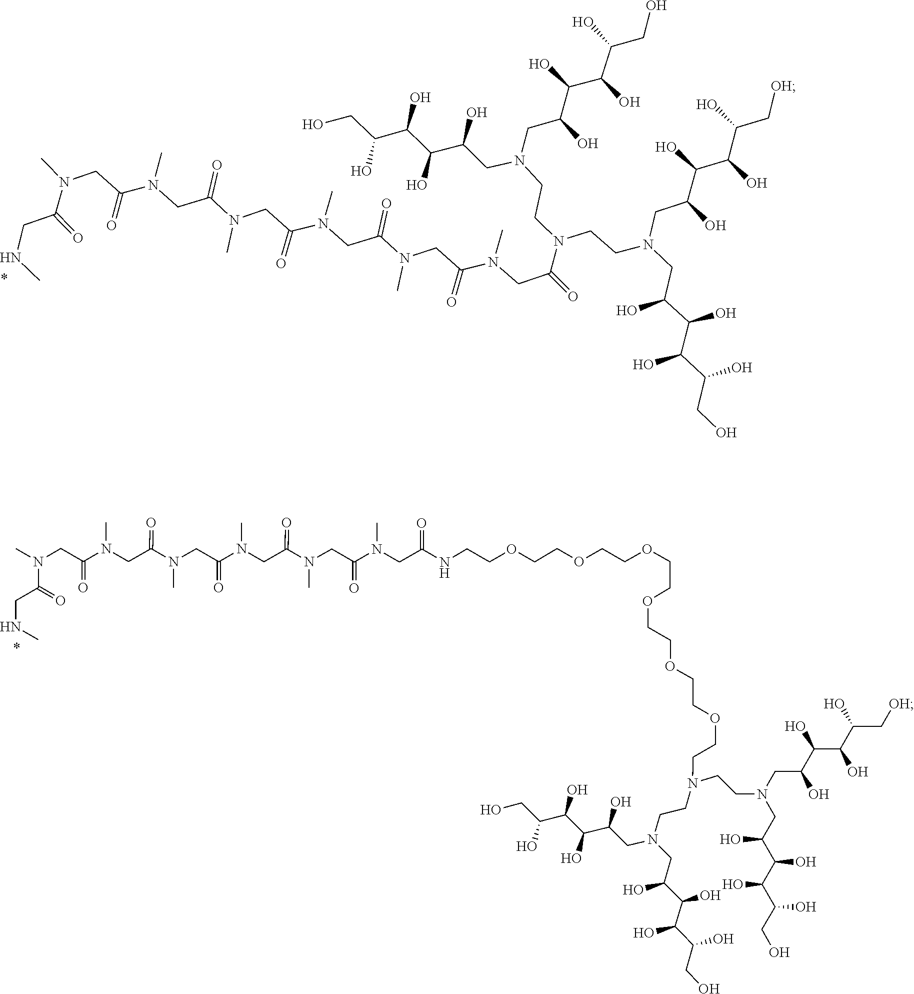 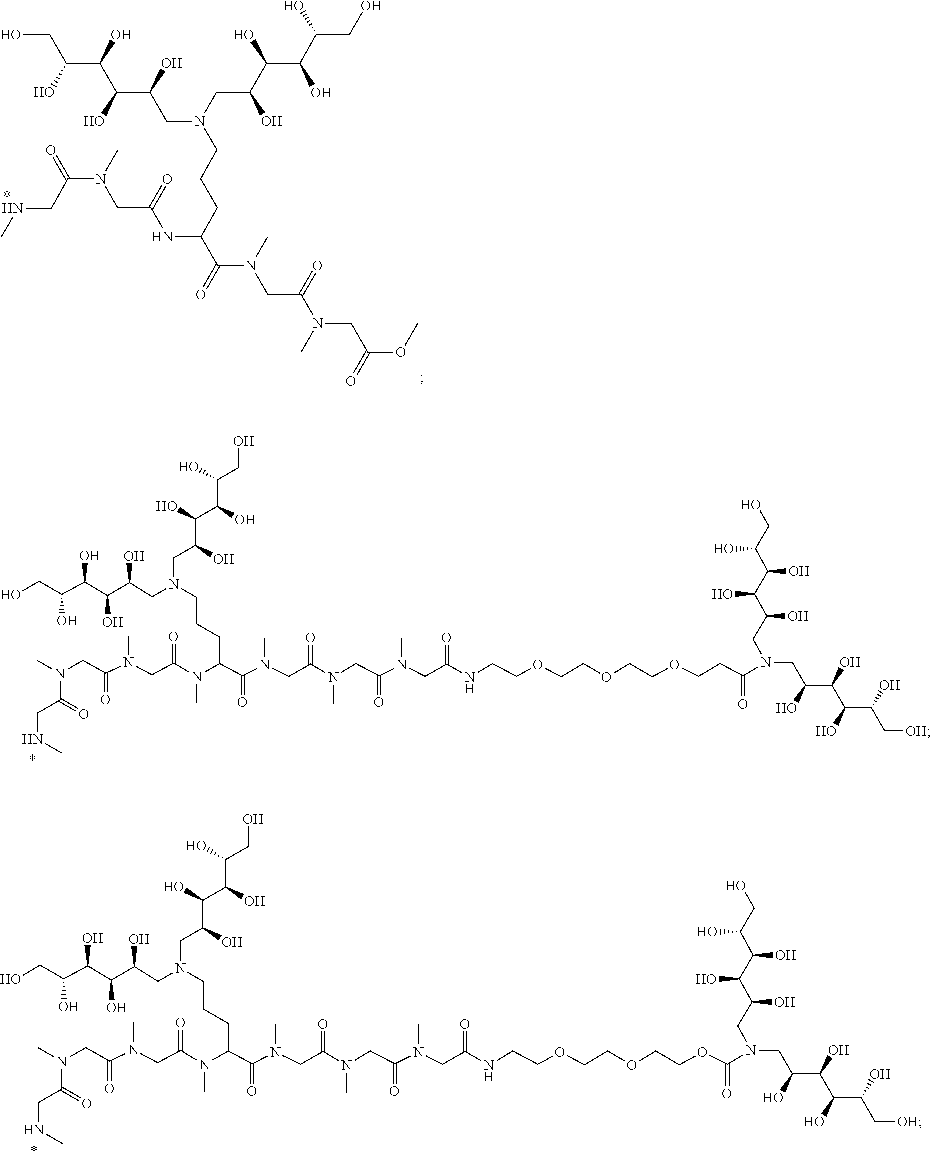 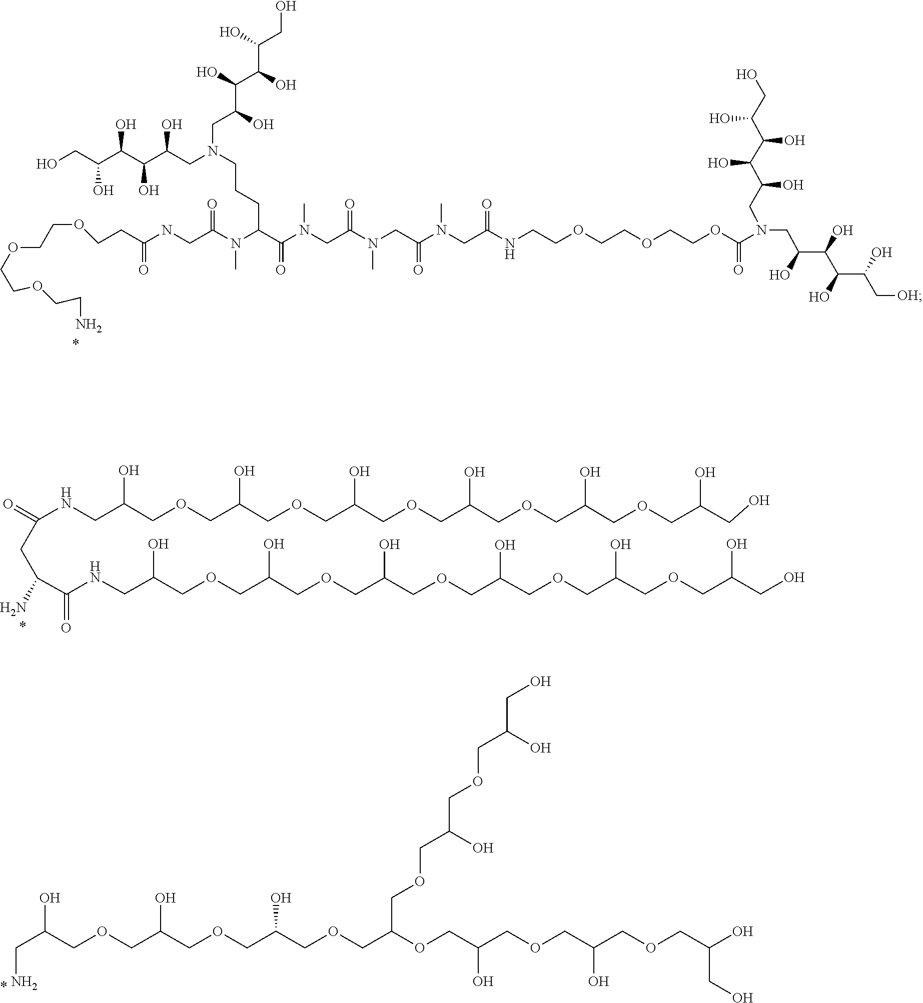 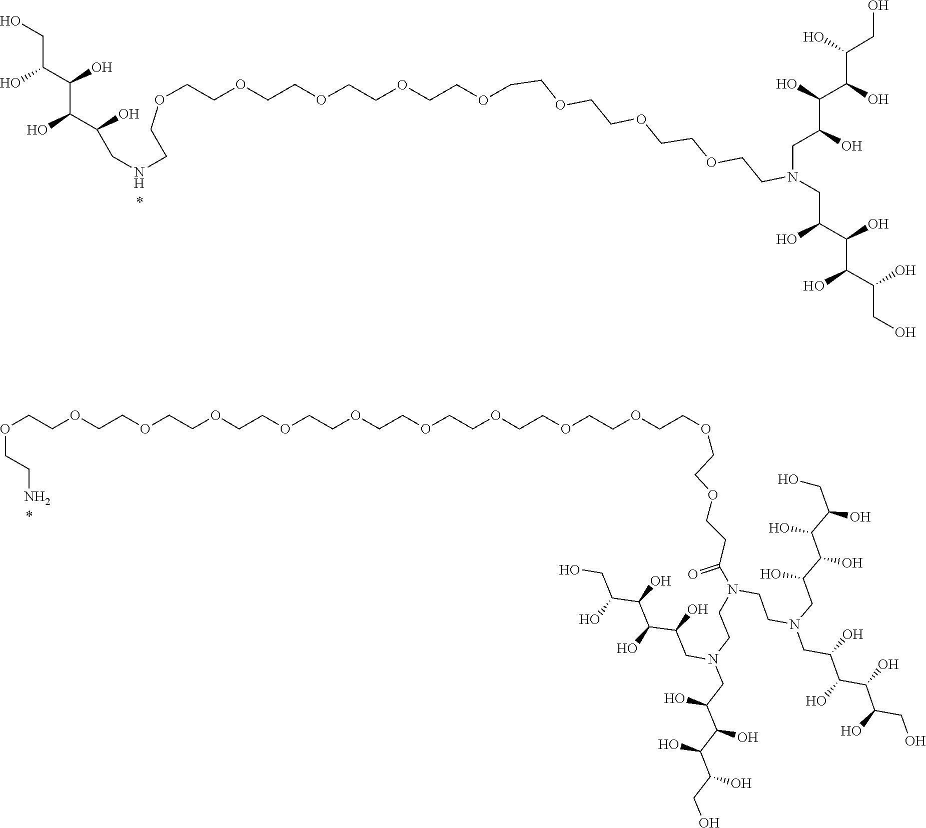 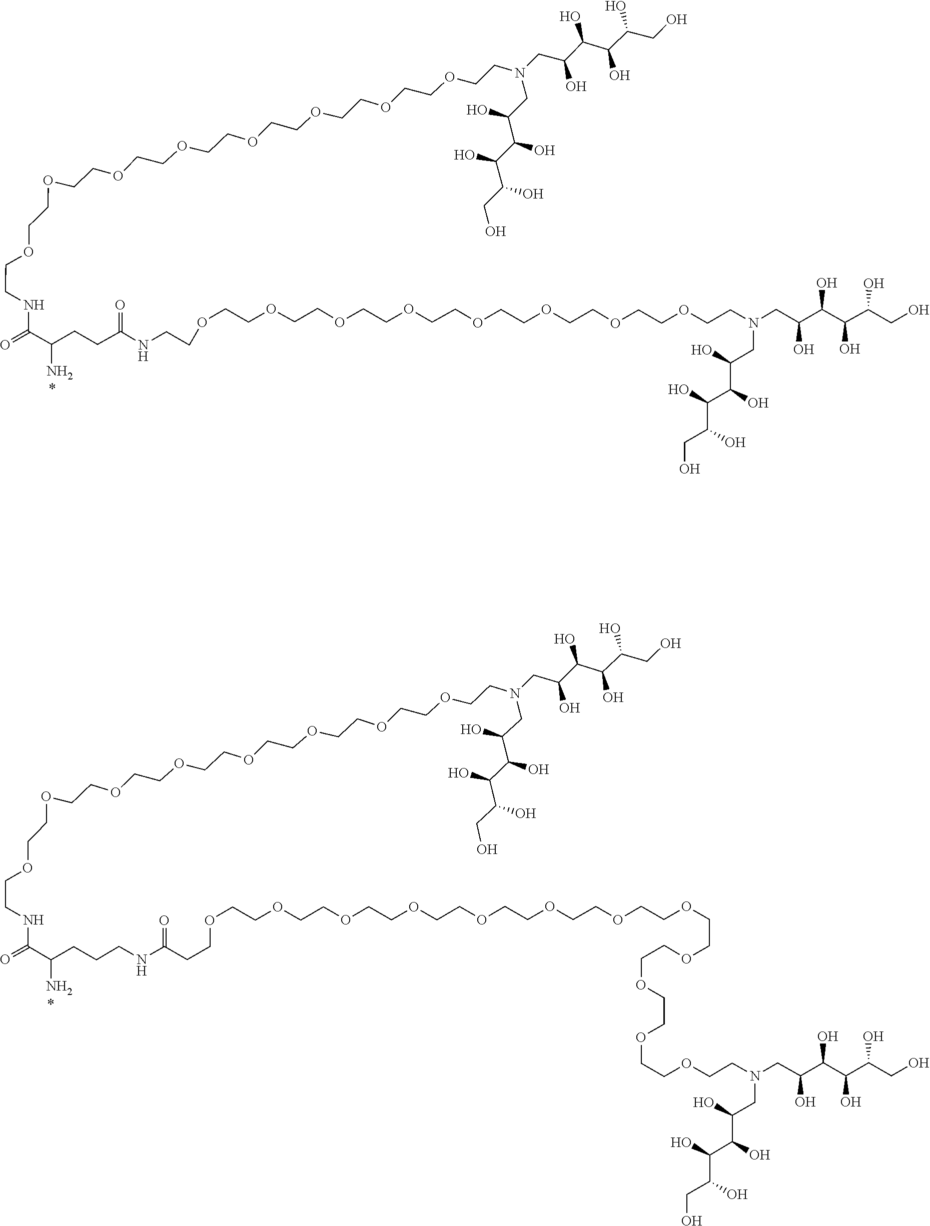 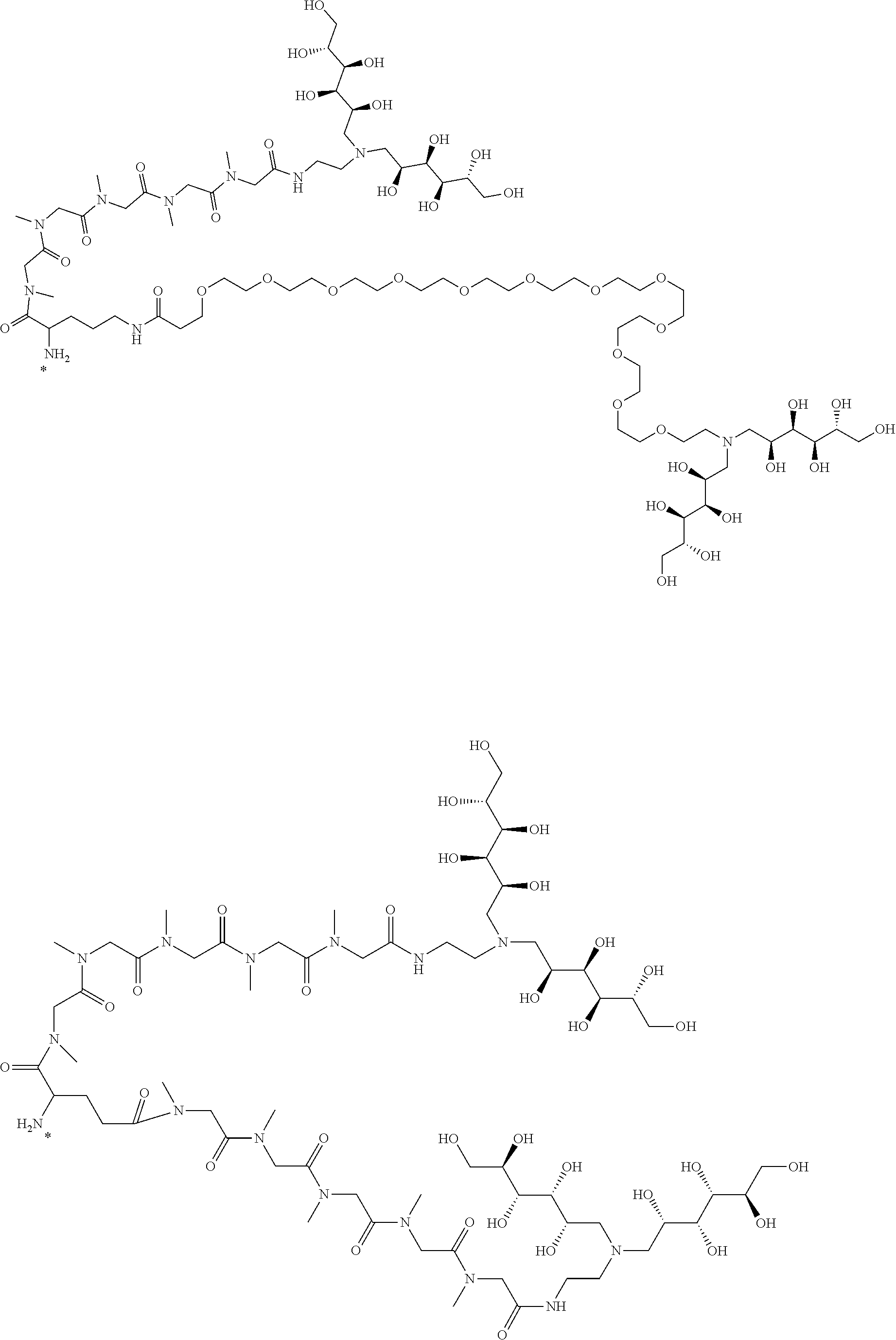 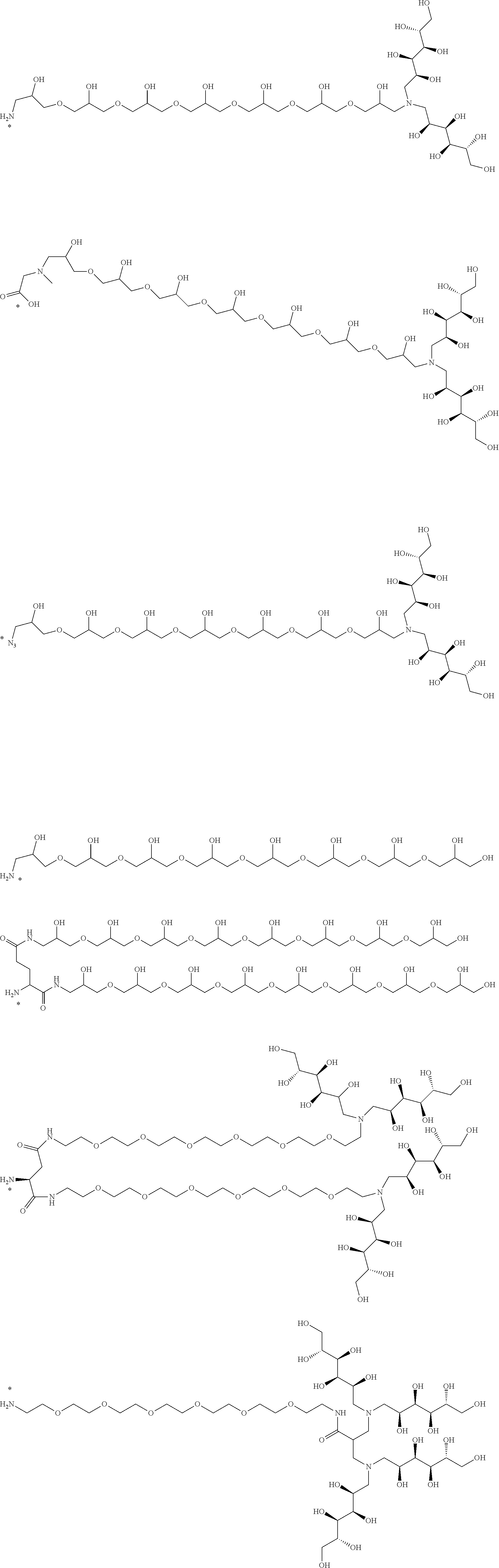 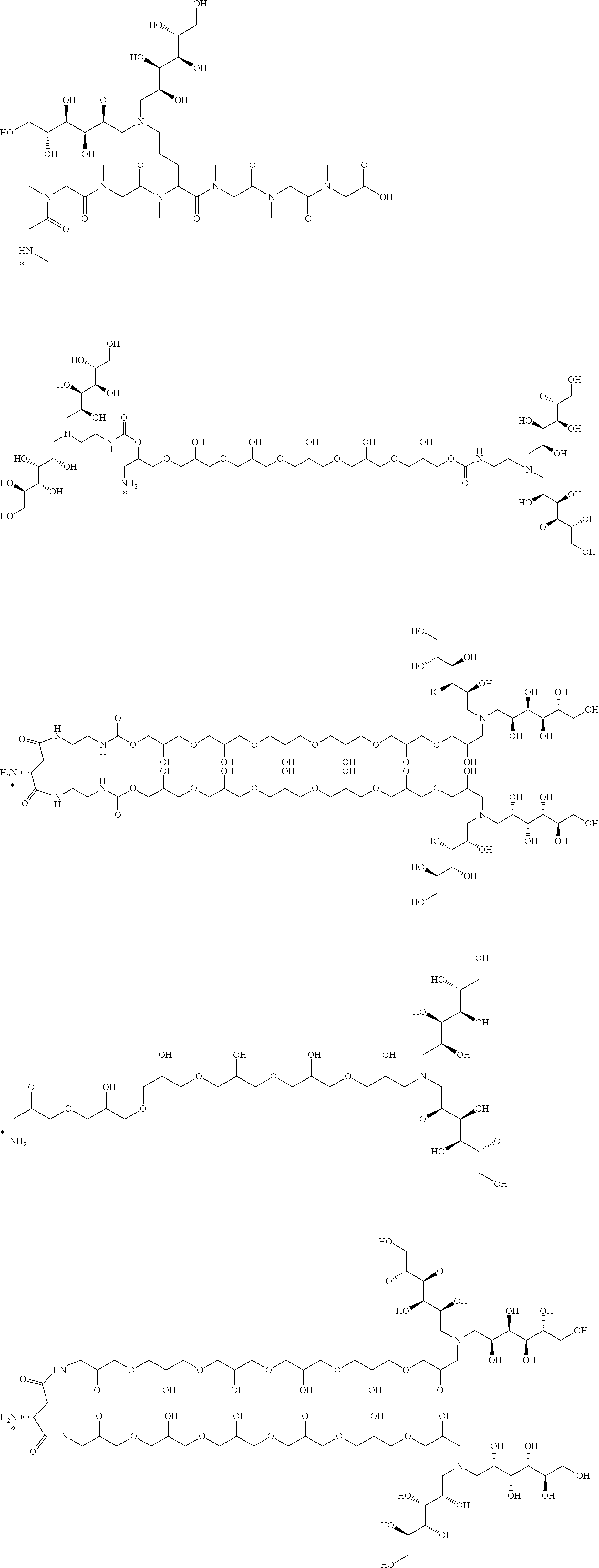 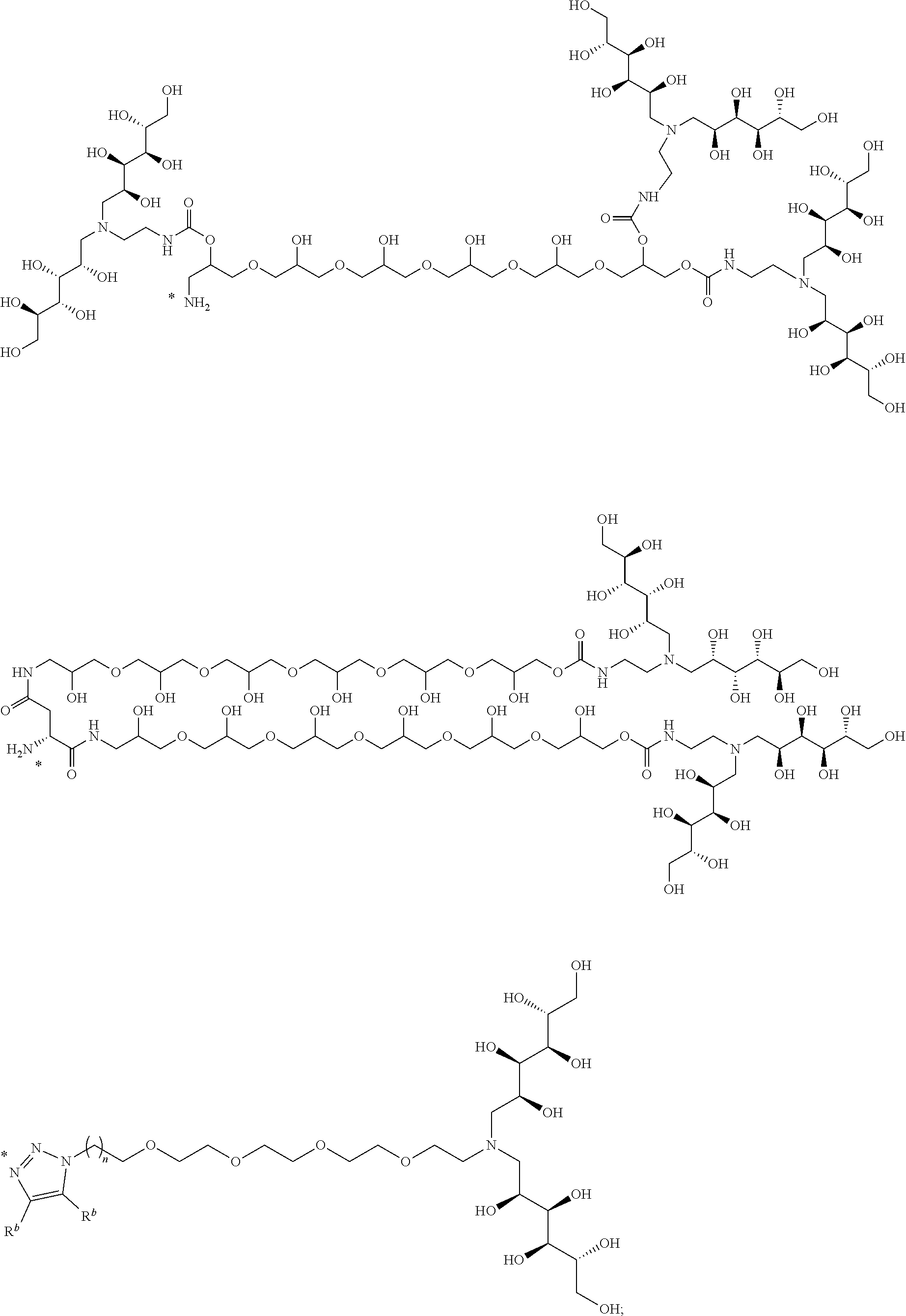 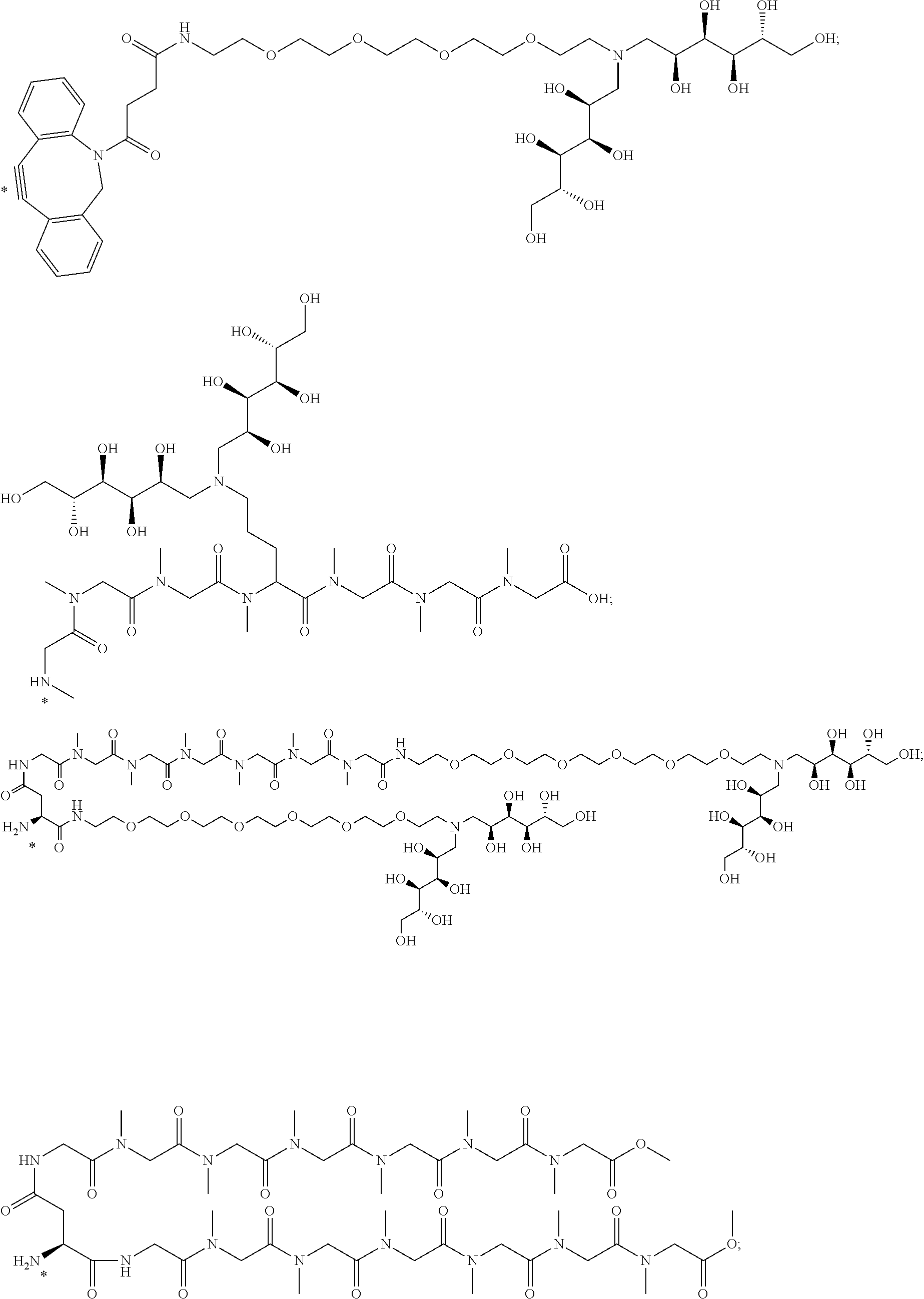 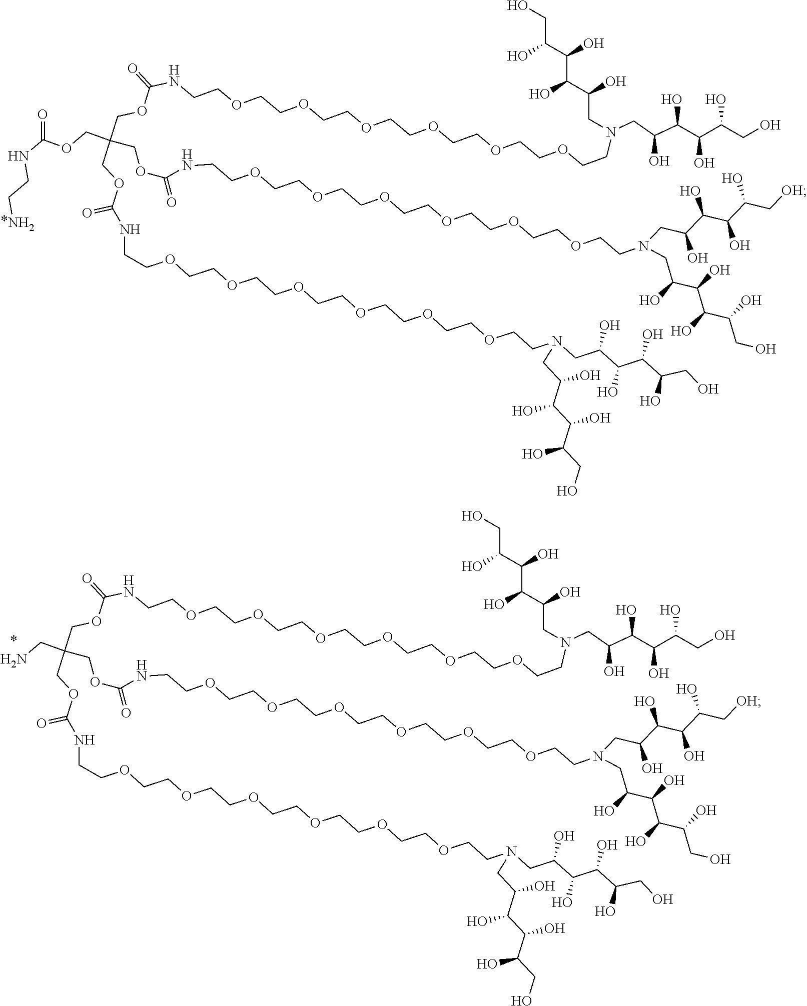 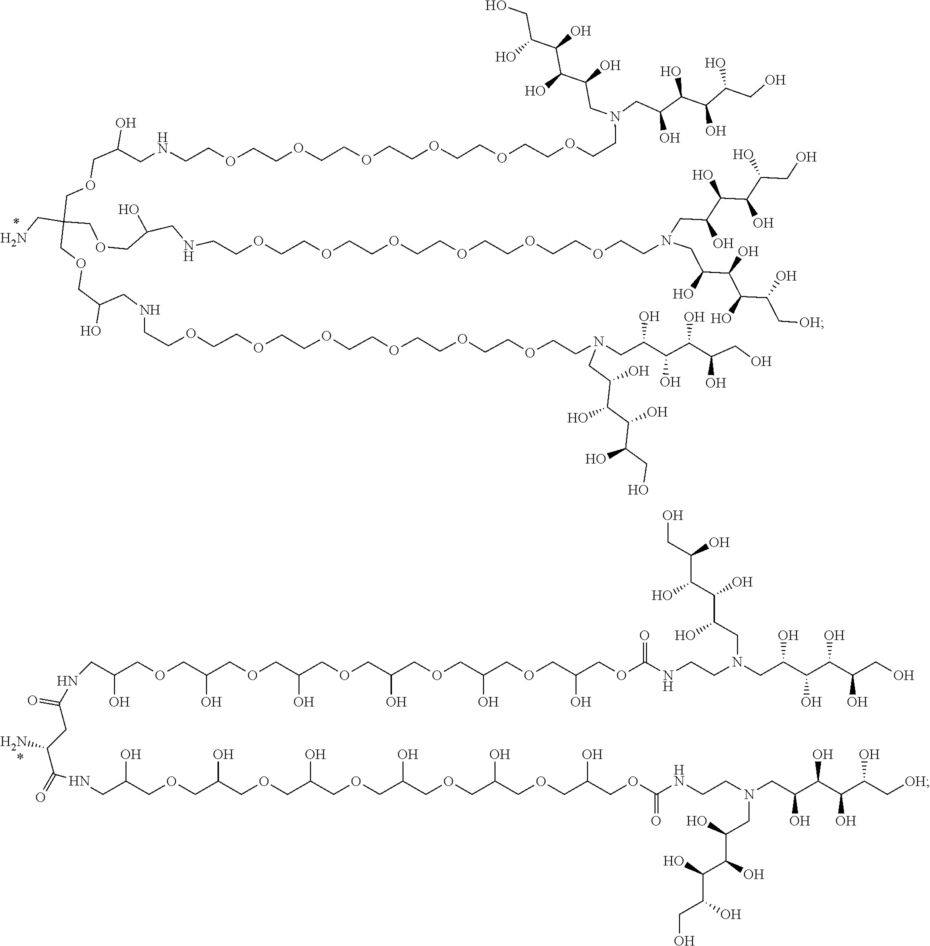 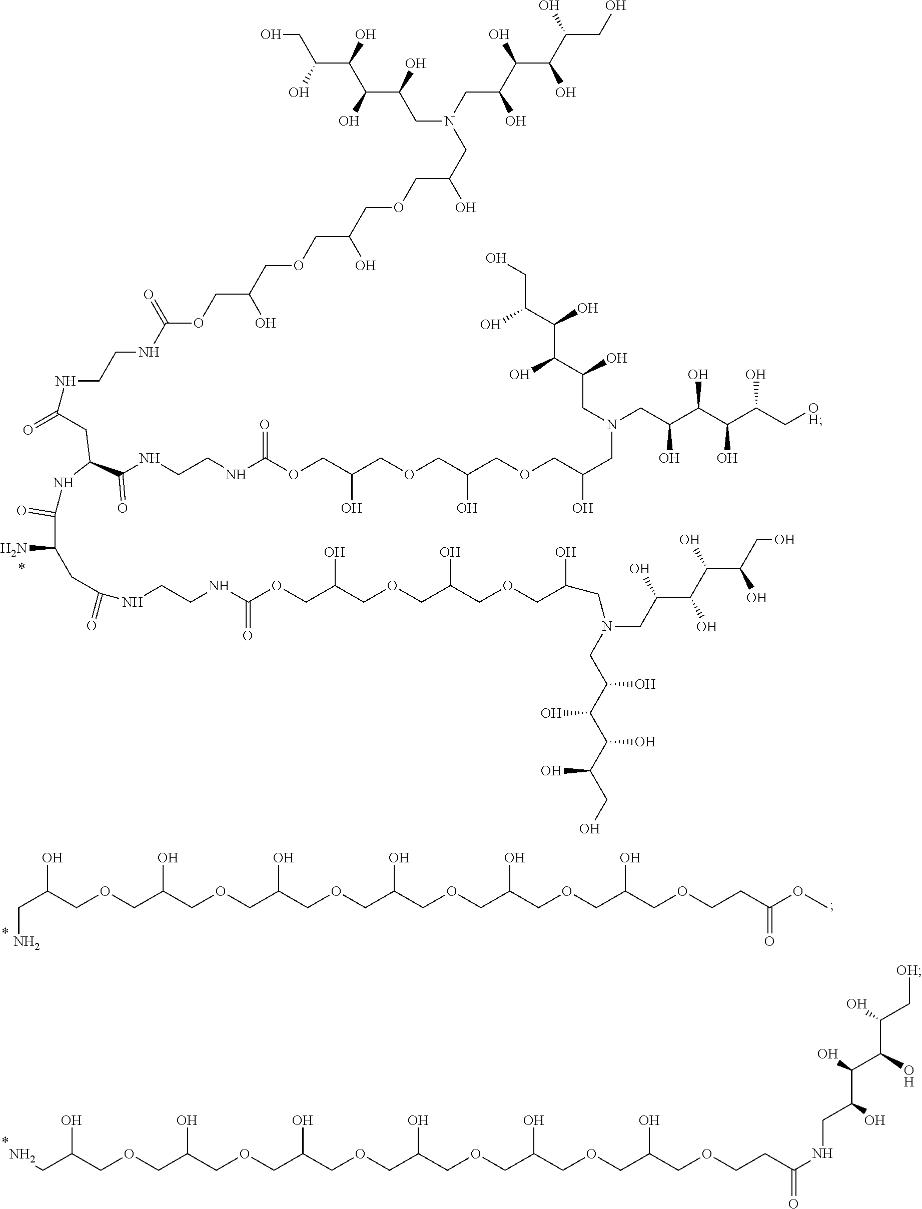 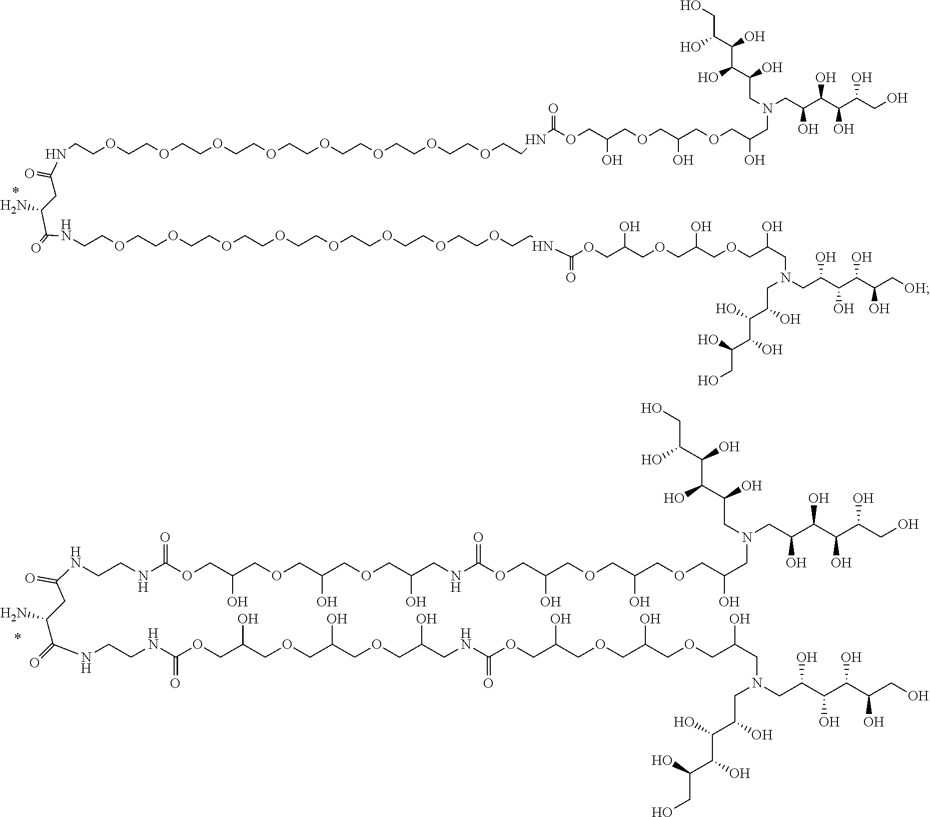 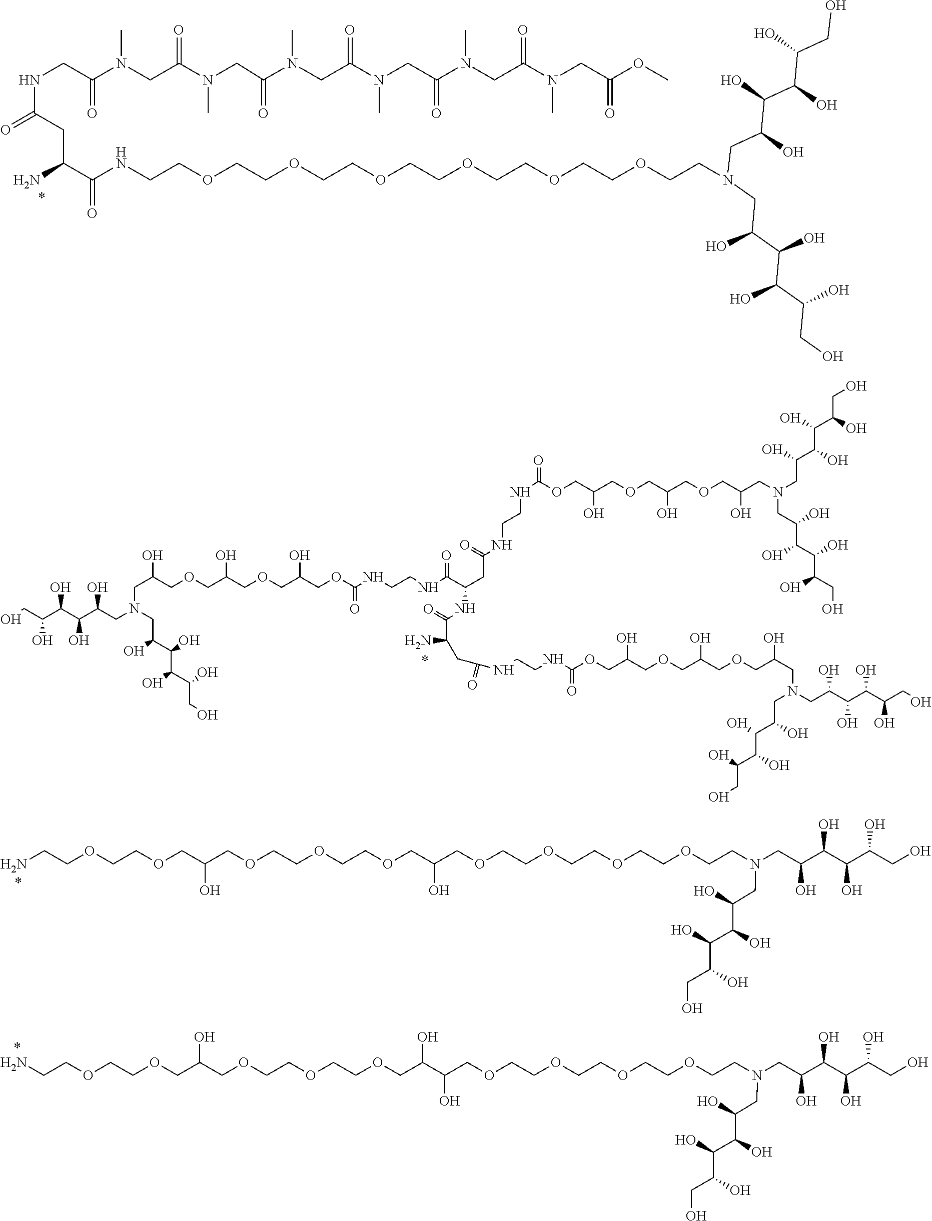 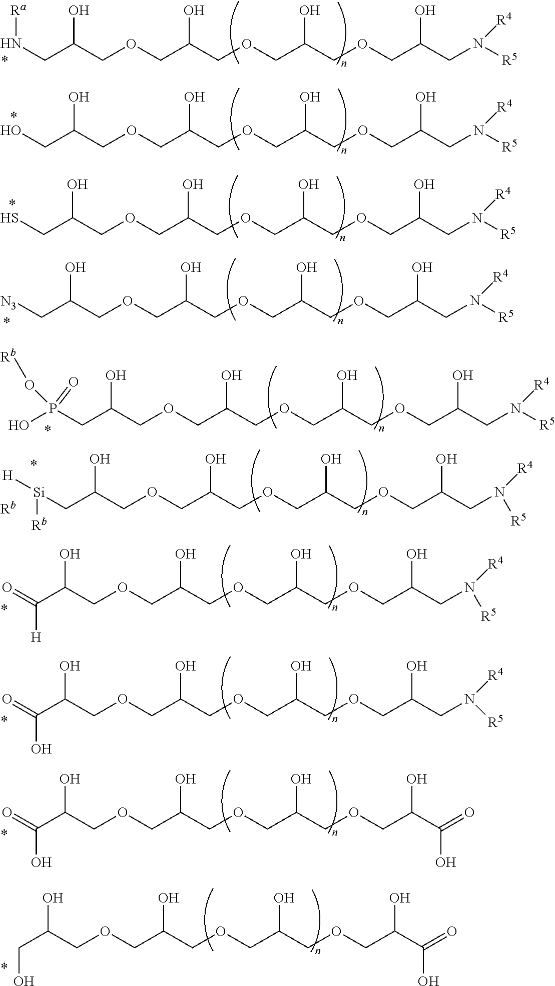 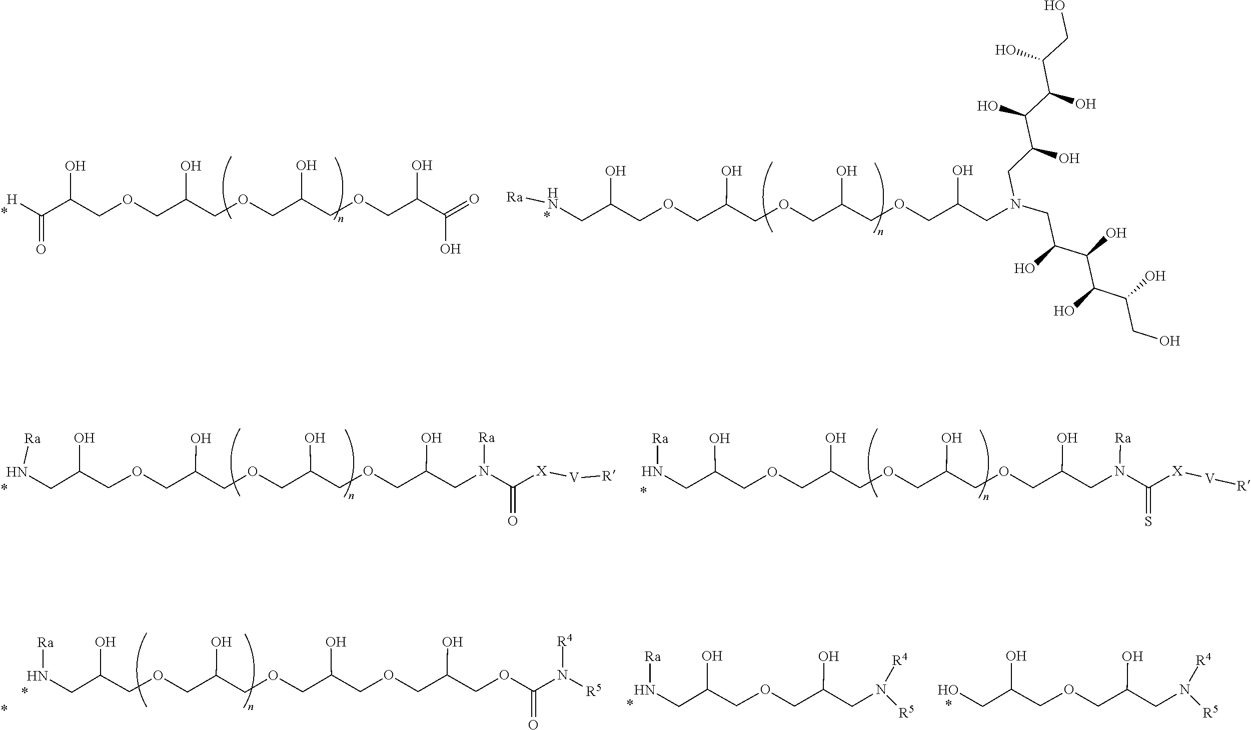 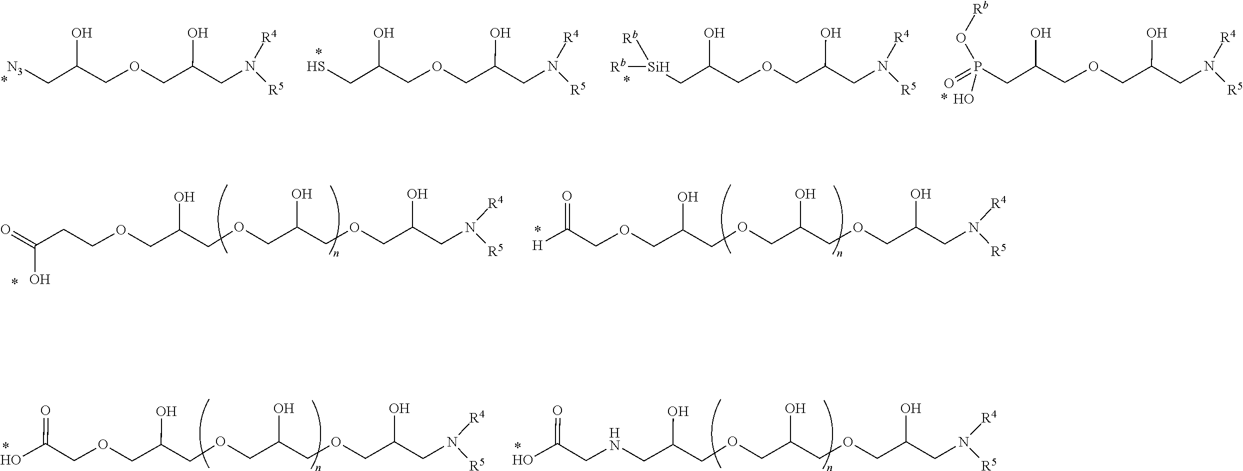 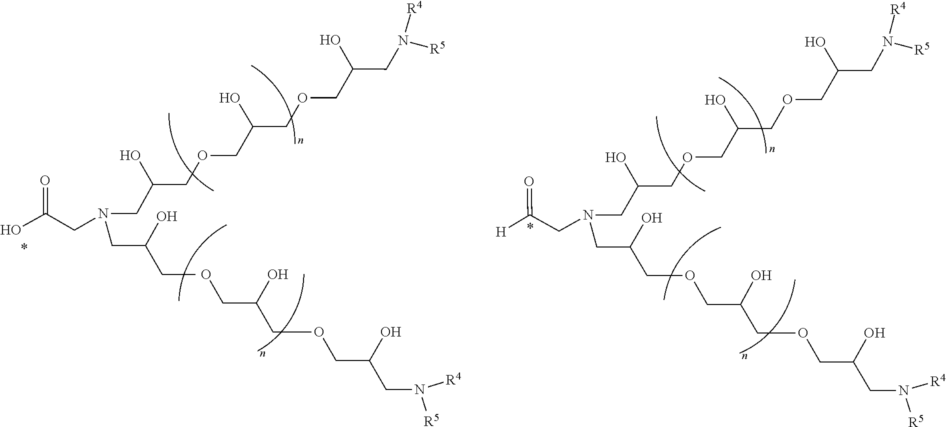 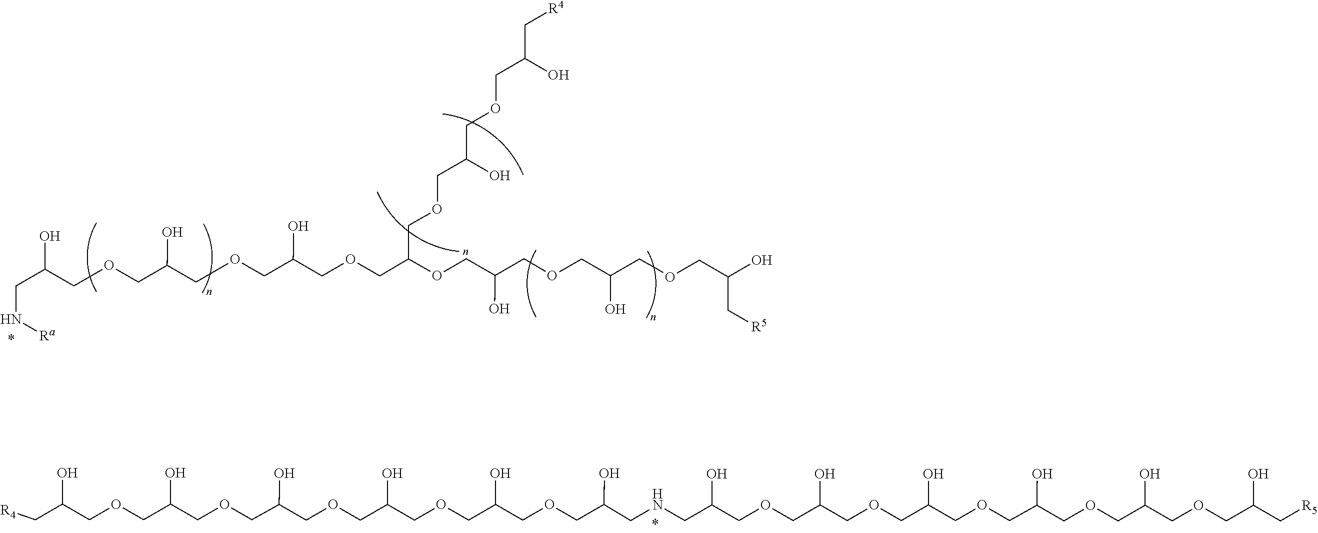 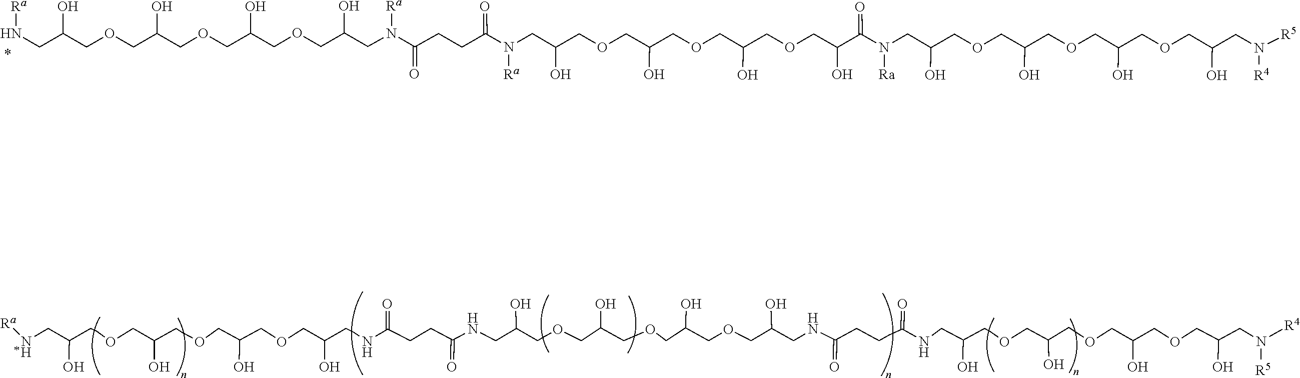 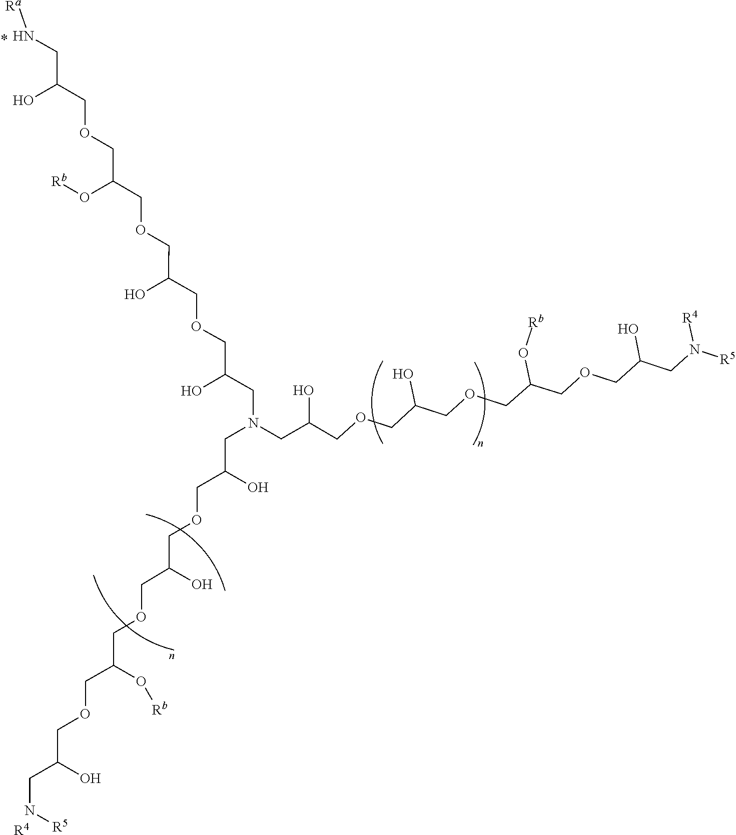 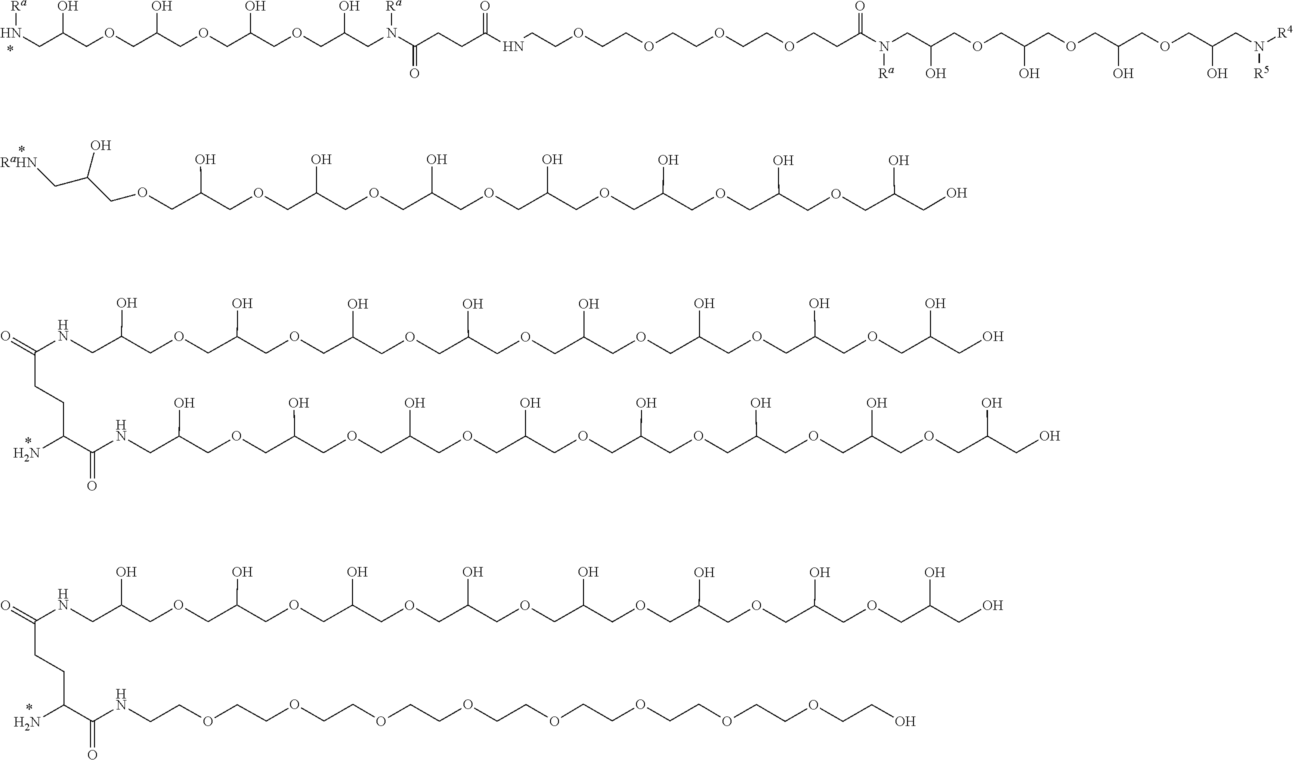 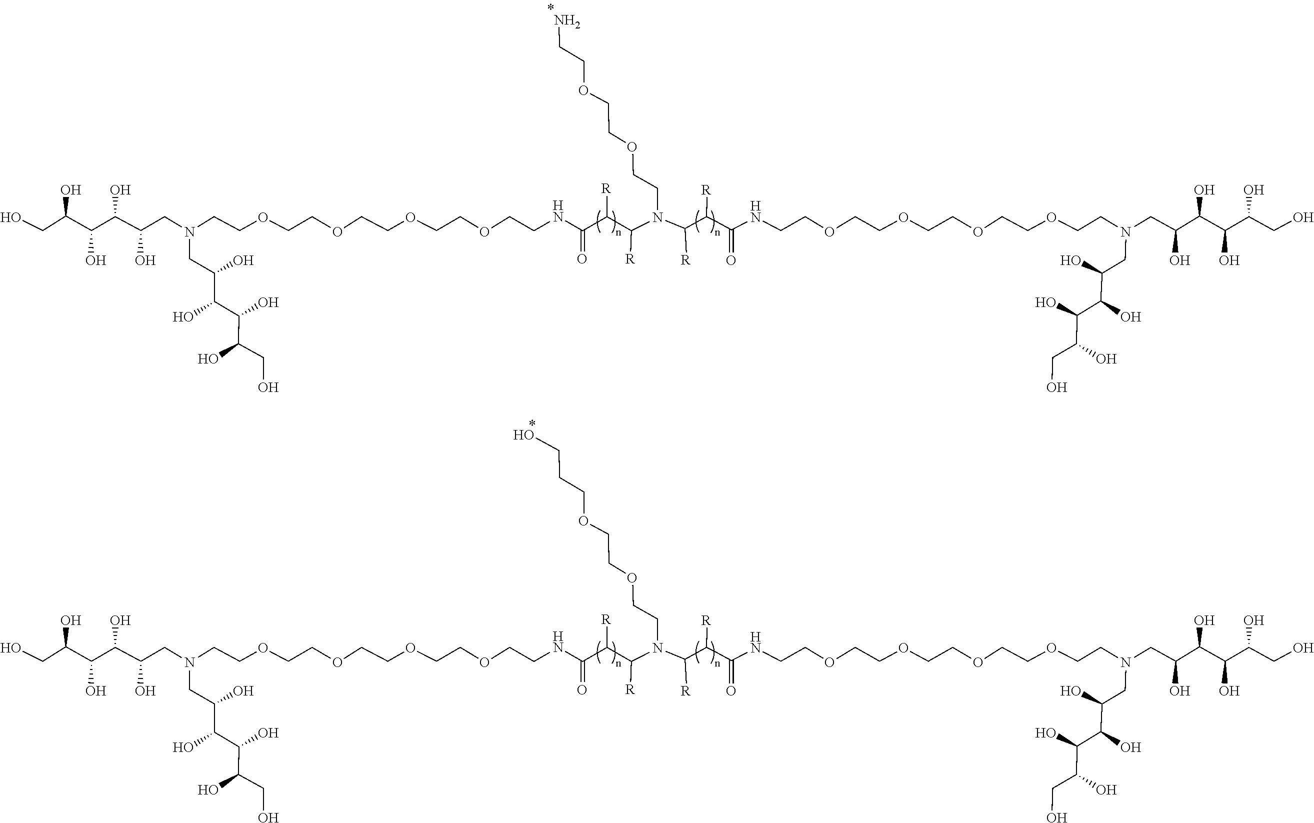 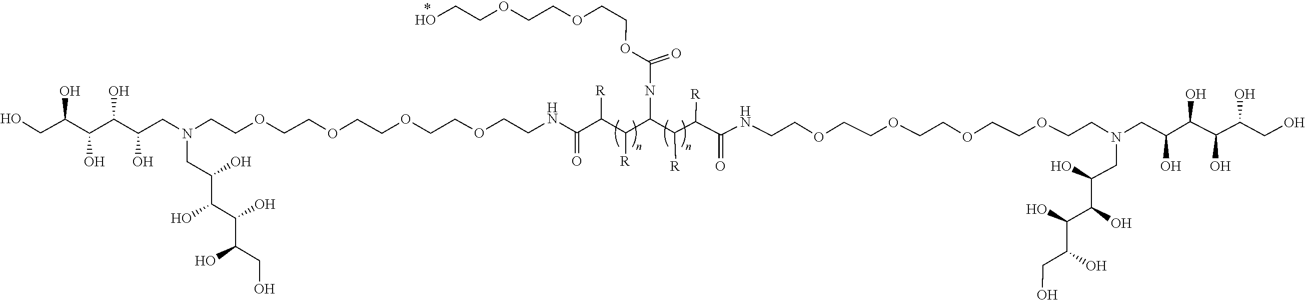
wherein:
(*) indicates the attachment site site β or to site R^{b}, or to the enzyme-cleavable group;
each R is independently H or C₁-C₆ alkyl;
R′ is H, C₁-C₆ alkyl, —N(R²⁴)(R²⁵) or —CO₂H;
each n is independently 1 to 12;
X is O, NR or —CH₂—;
V is bond or C₁-C₆ alkyl;
one of R²⁴ and R²⁵ is selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is a sugar unit of formula (XII) or (XIII); and the other of R²⁴ and R²⁵ is selected from H; polyhydroxyl group; substituted polyhydroxyl group; —C(O)-polyhydroxyl group; substituted —C(O)— polyhydroxyl group; substituted —C(O)—C₁-C₈ alkyl; a chelator; —C(O)—R²⁸, where R²⁸ is a sugar unit of formula (XII) or (XIII); and polyethylene glycol, optionally having 1 to 24 ethylene glycol subunits, provided that R²⁴ and R²⁵ are not both H.

54. The conjugate of claim 30, comprising a polar group selected from the following, or a stereoisomer or salt thereof: 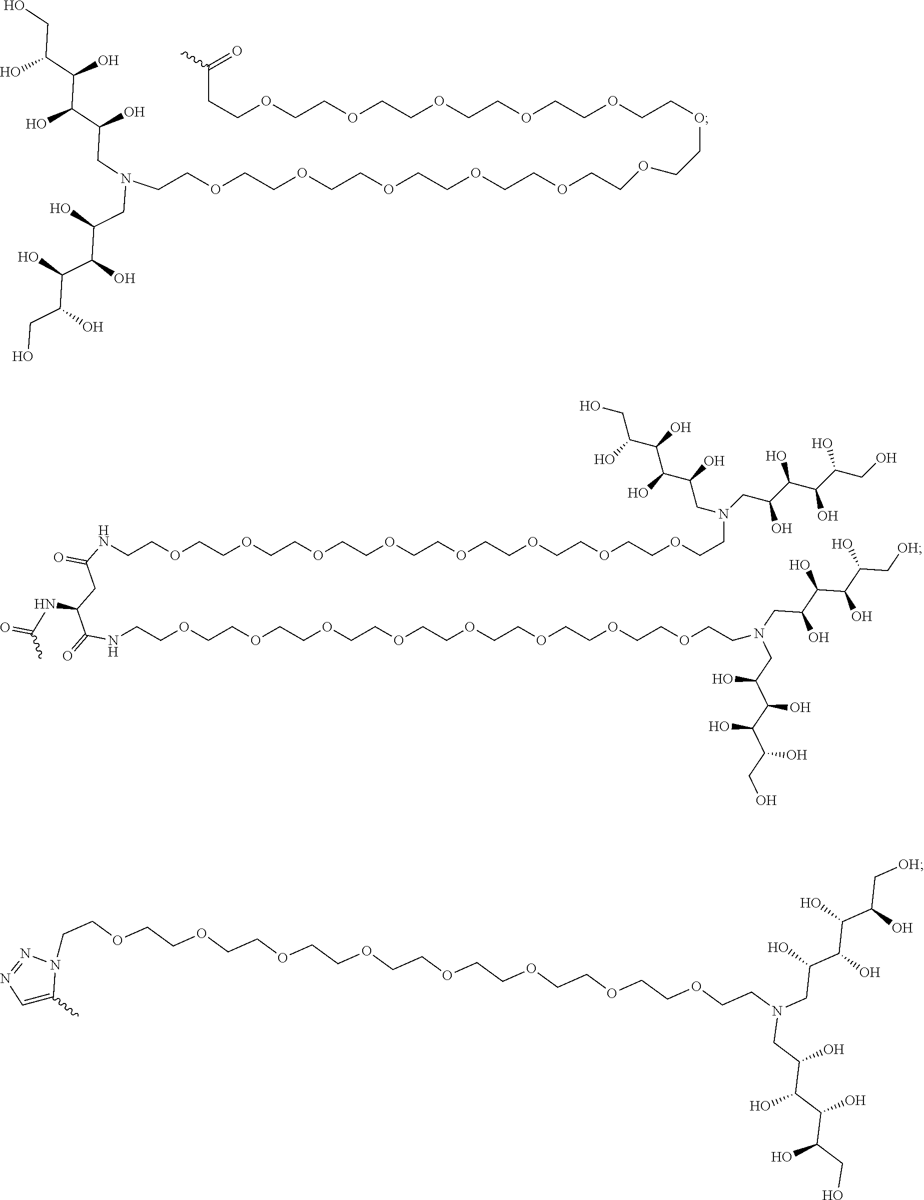 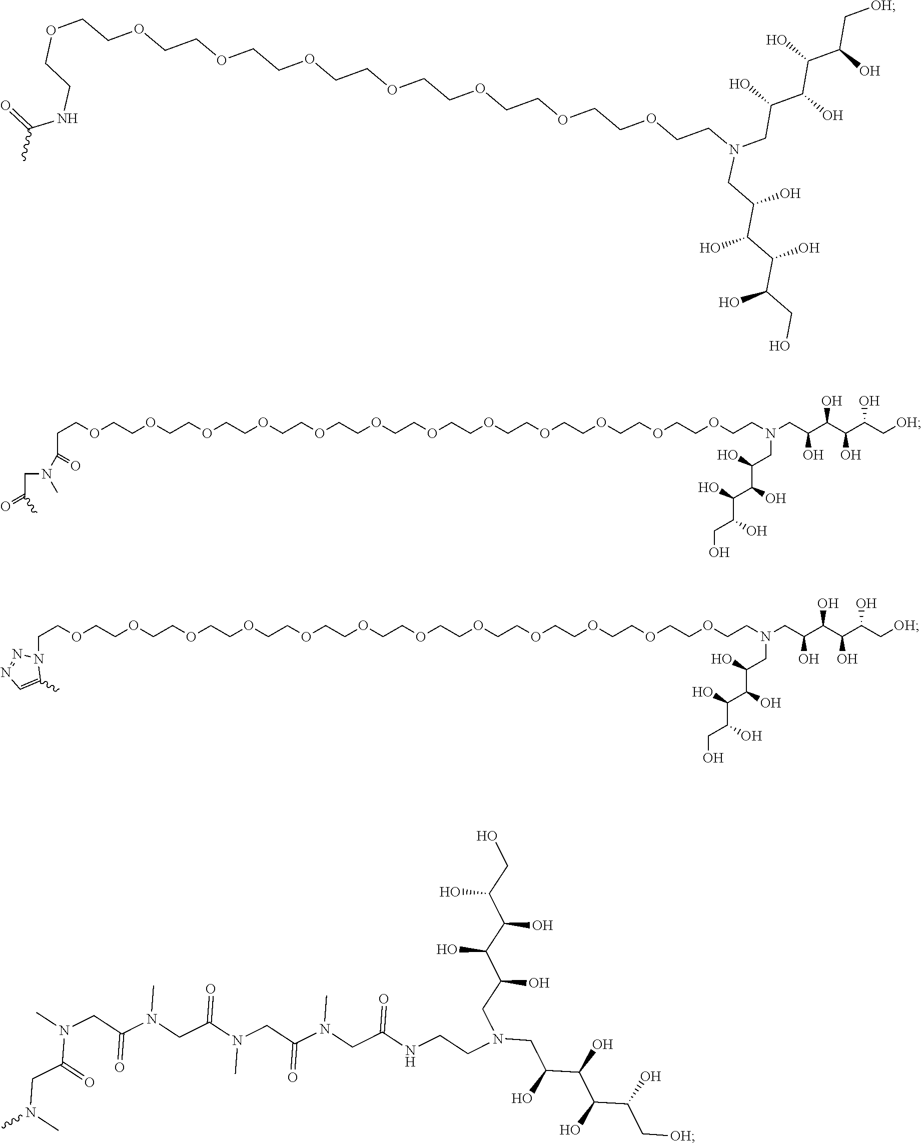 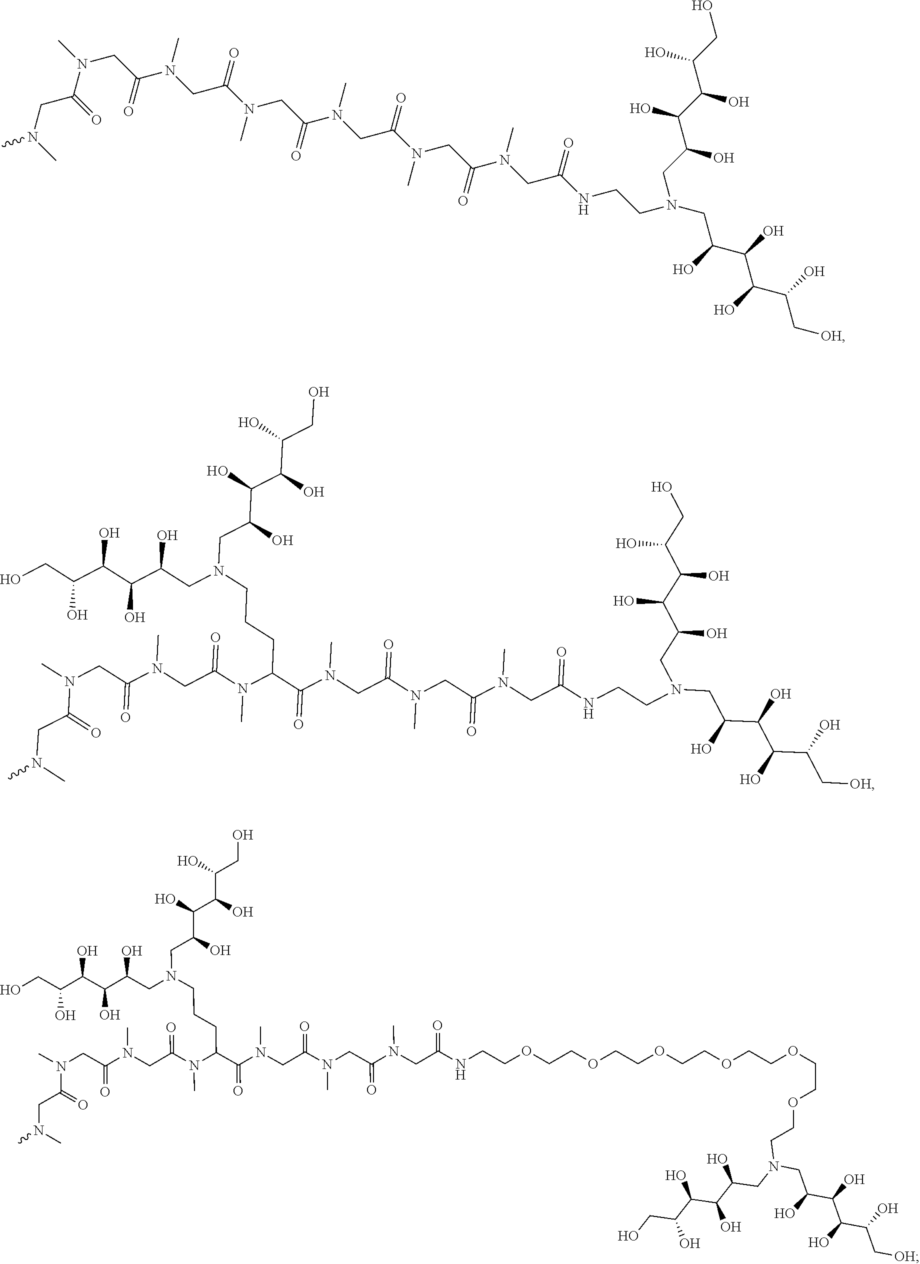 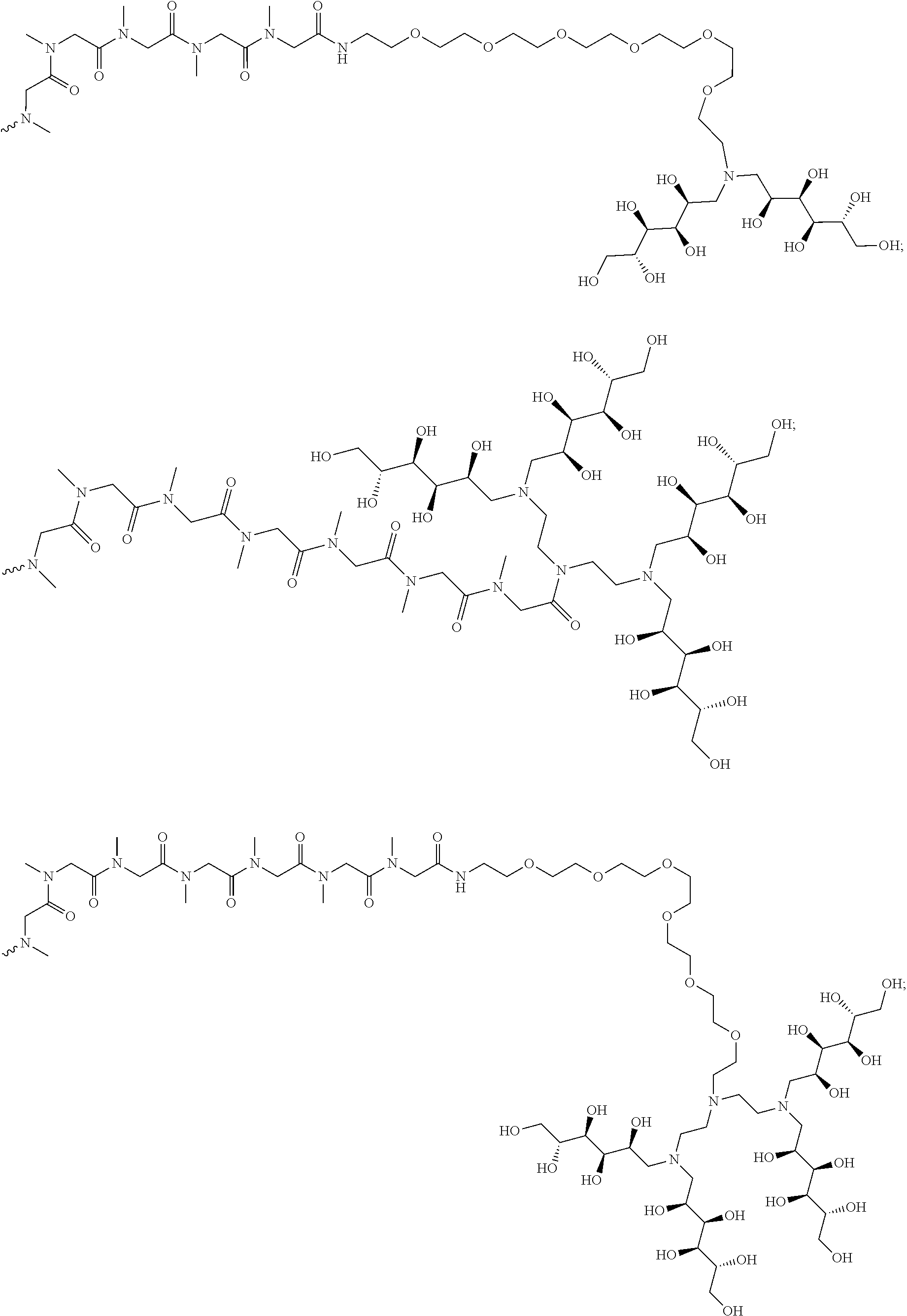 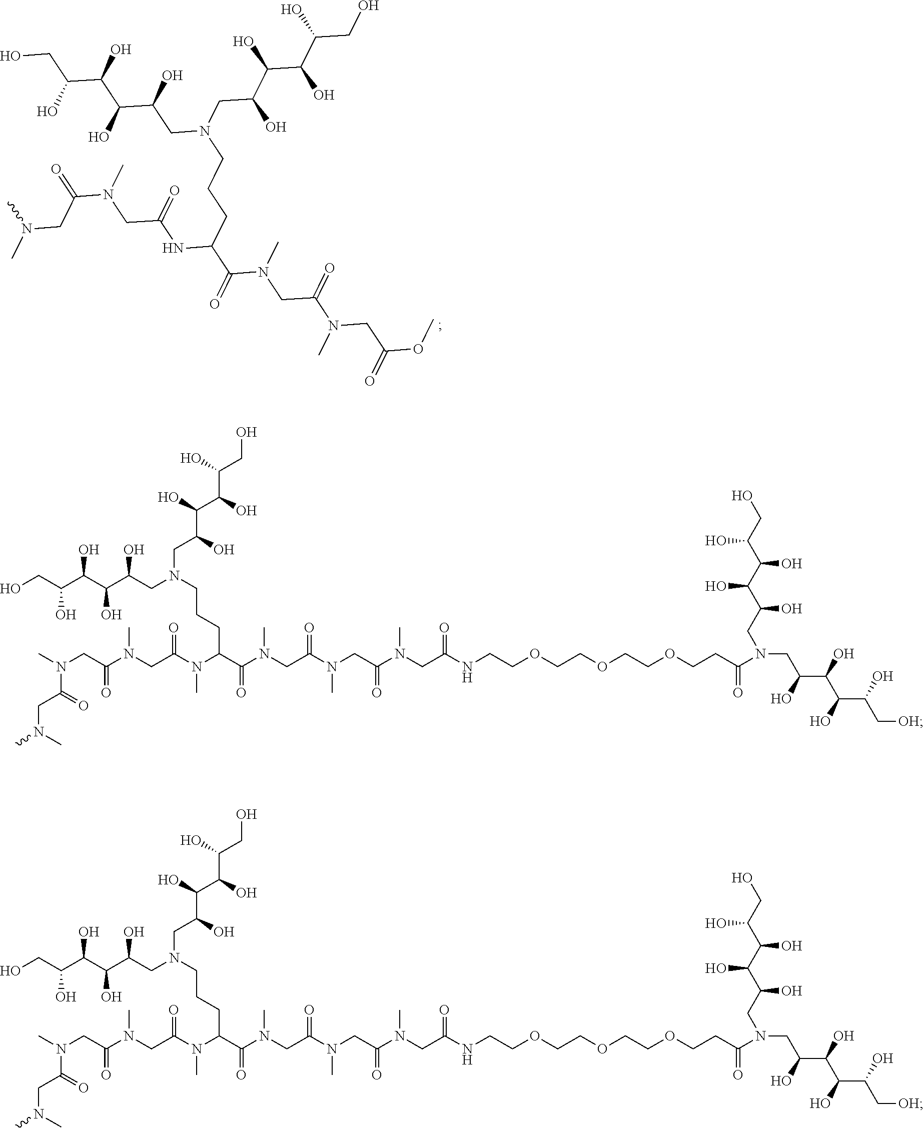 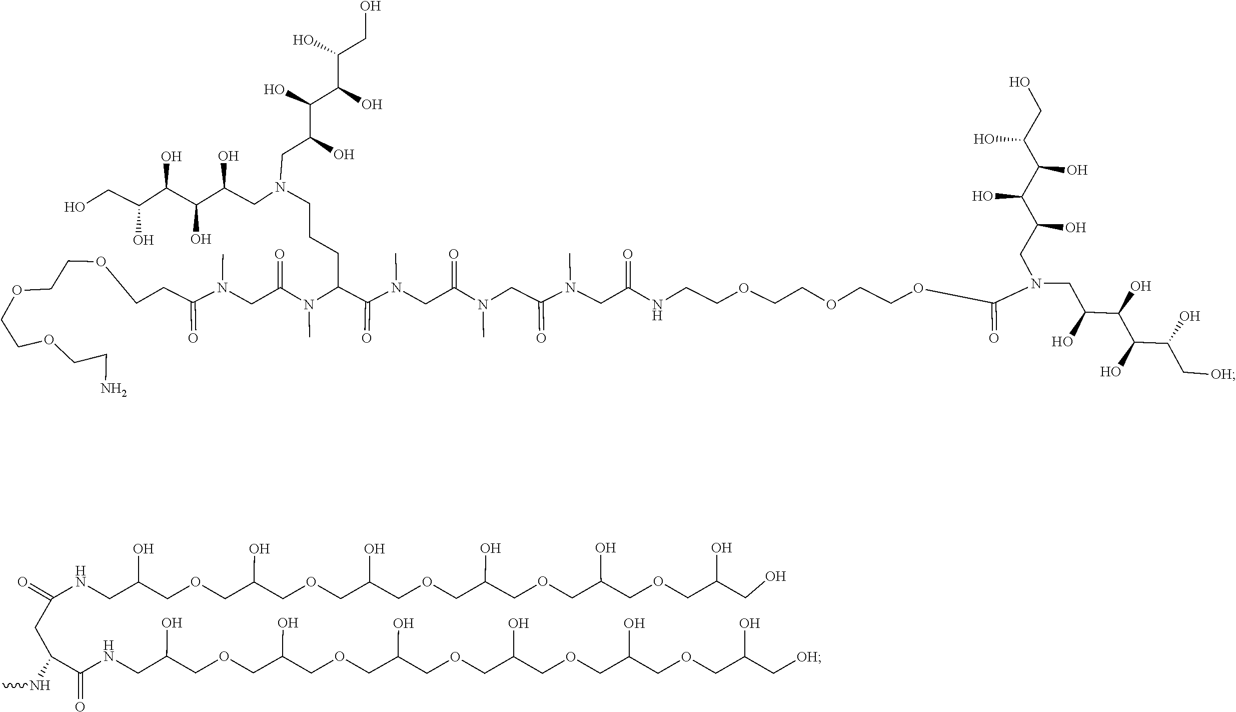 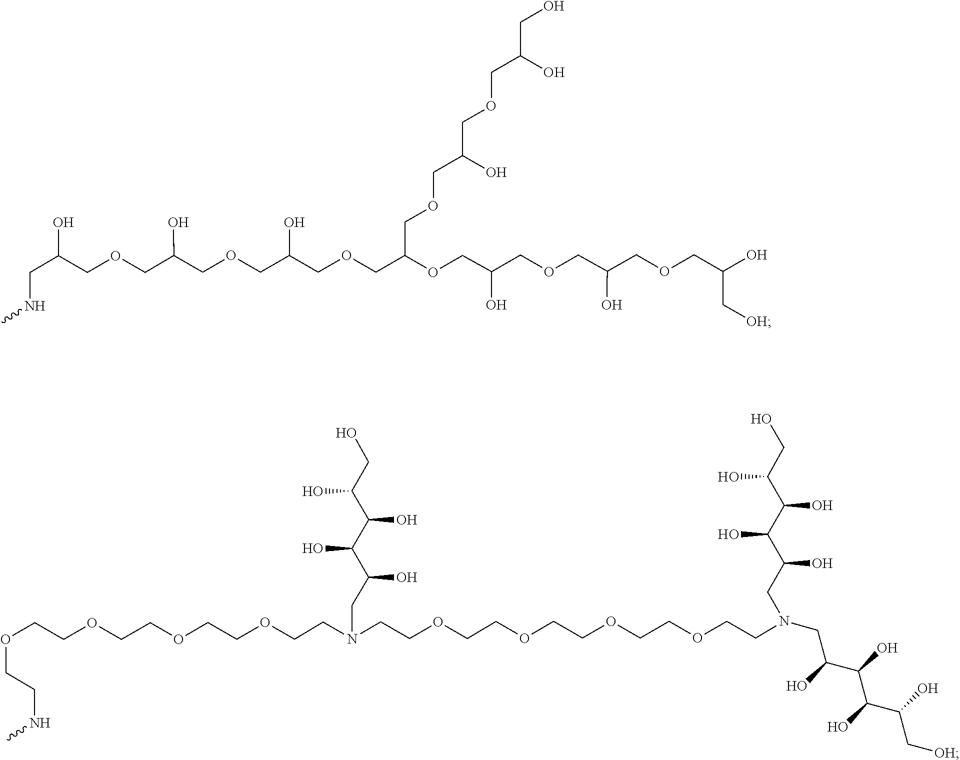 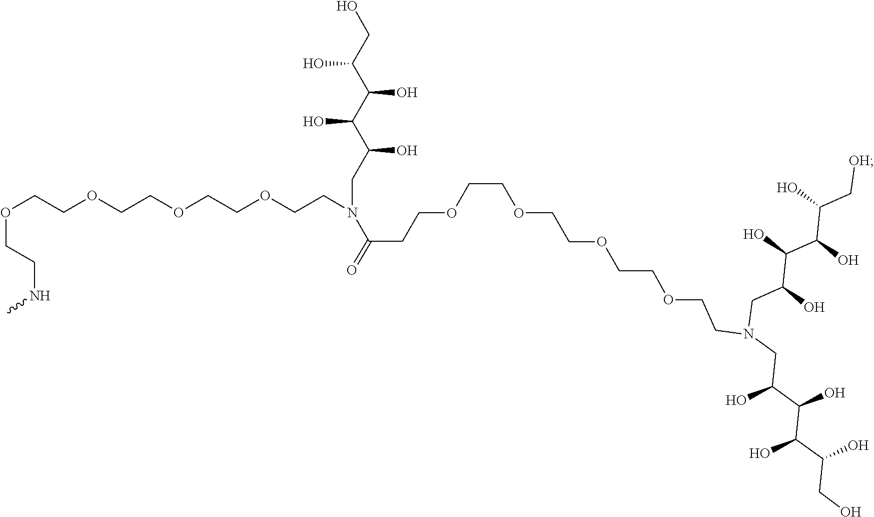 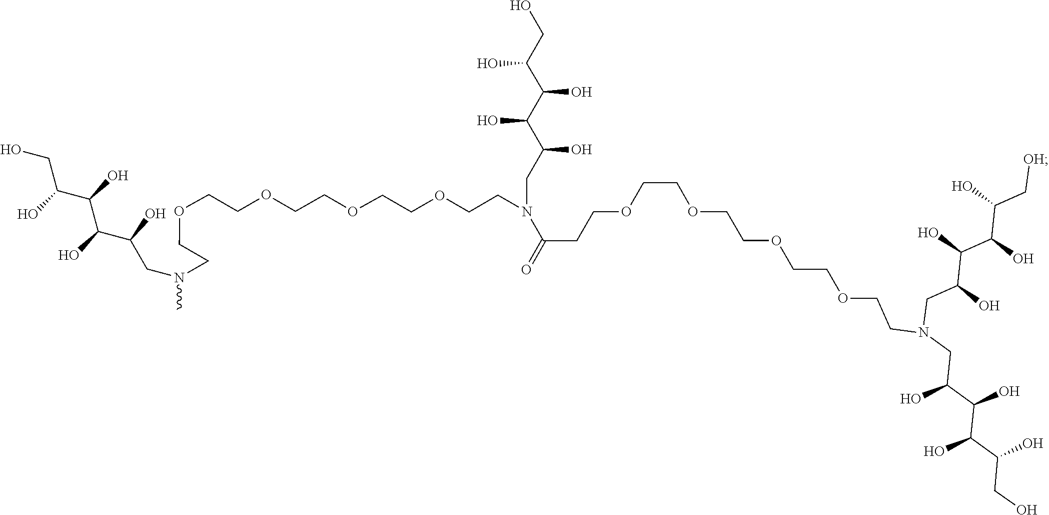 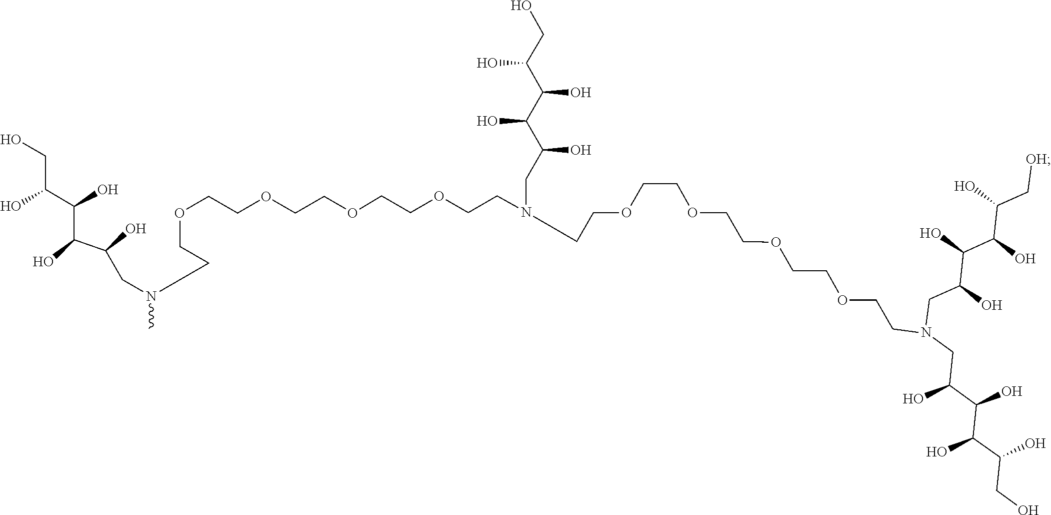 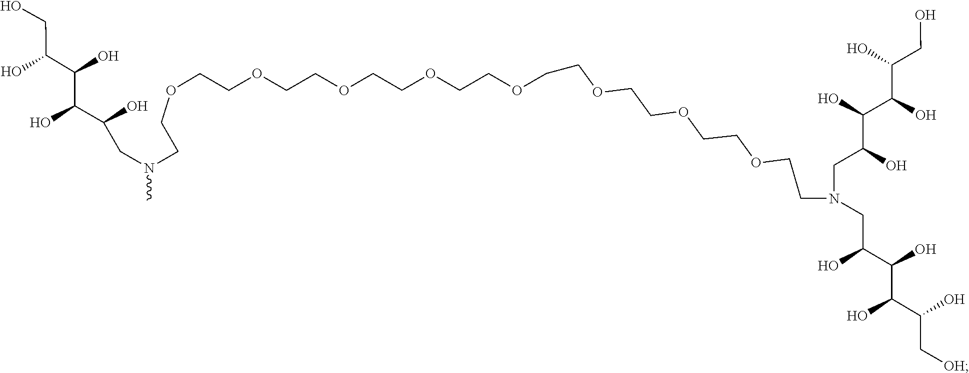 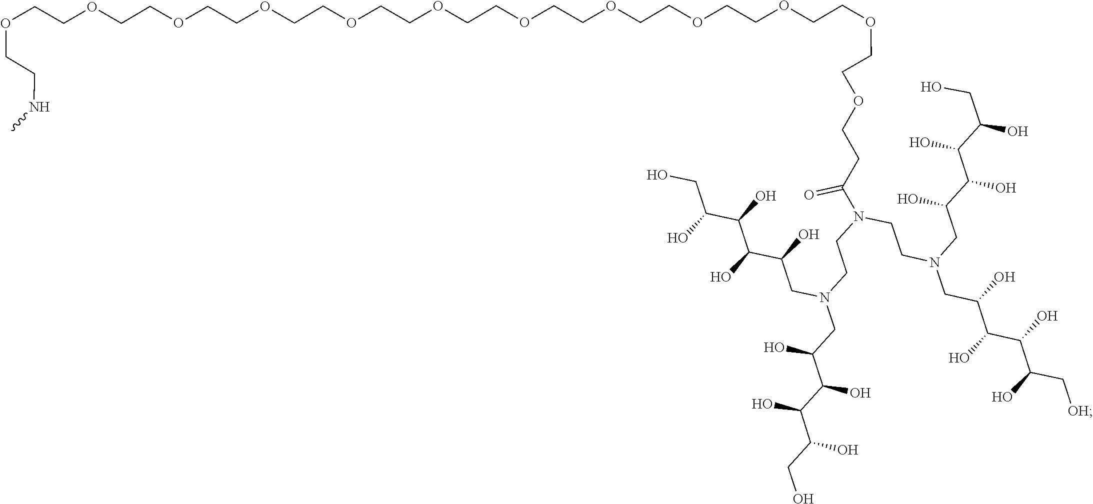 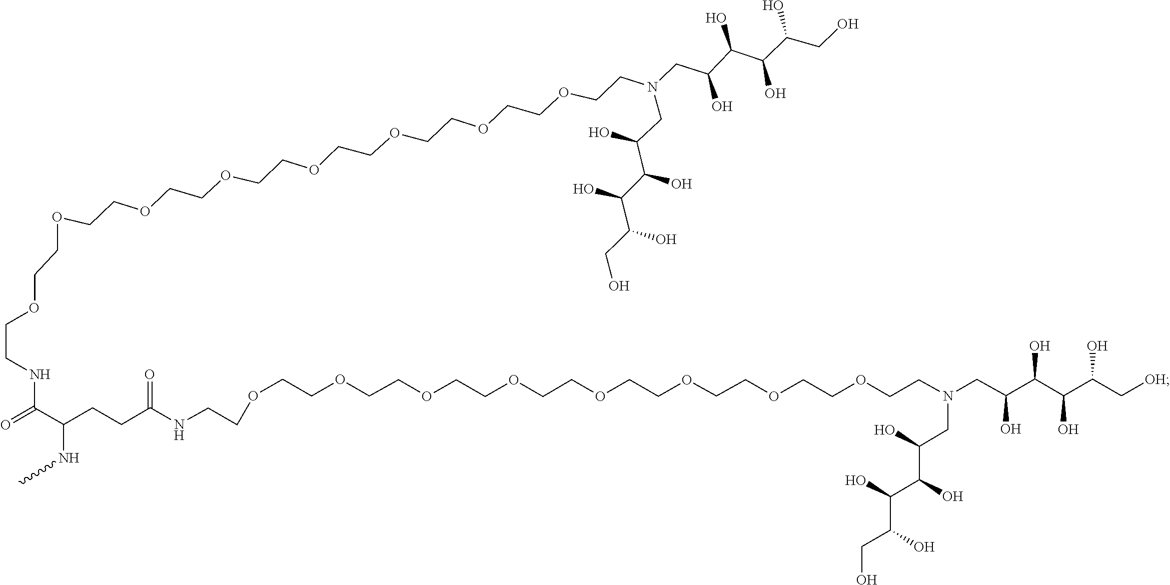 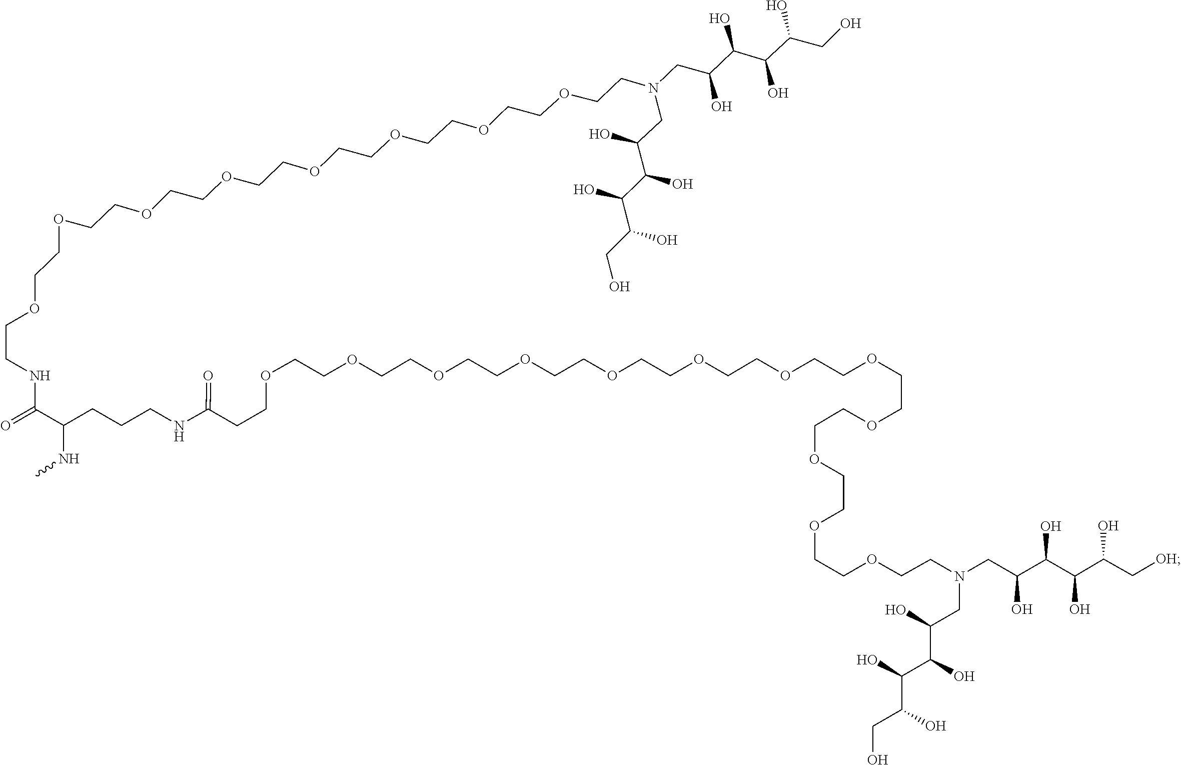 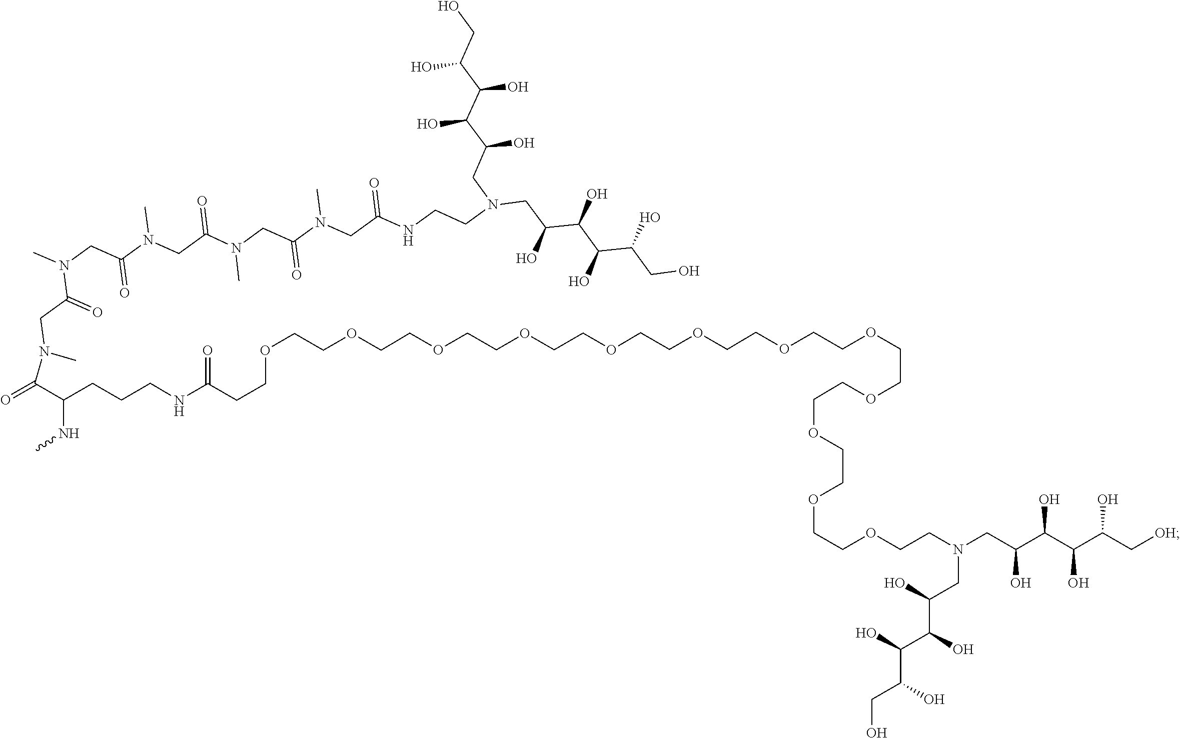 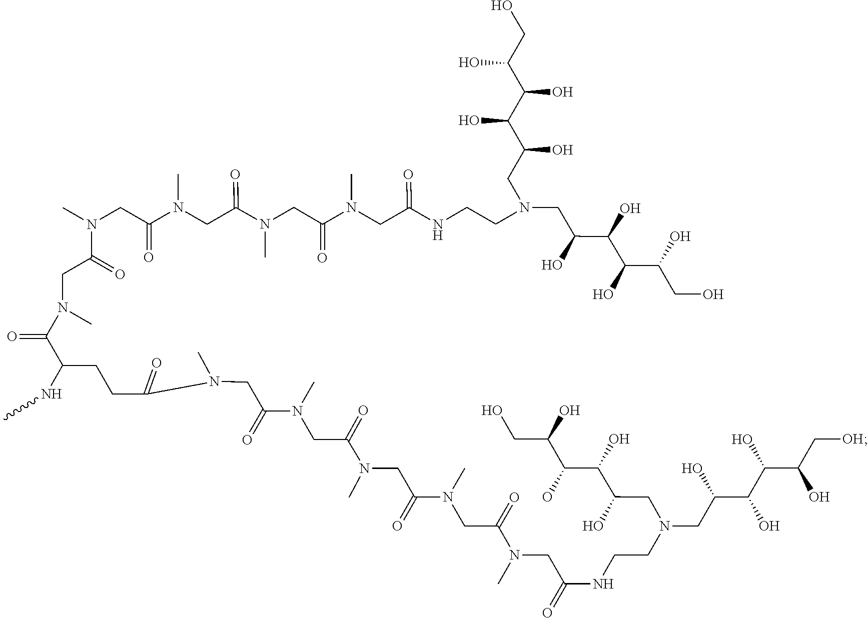 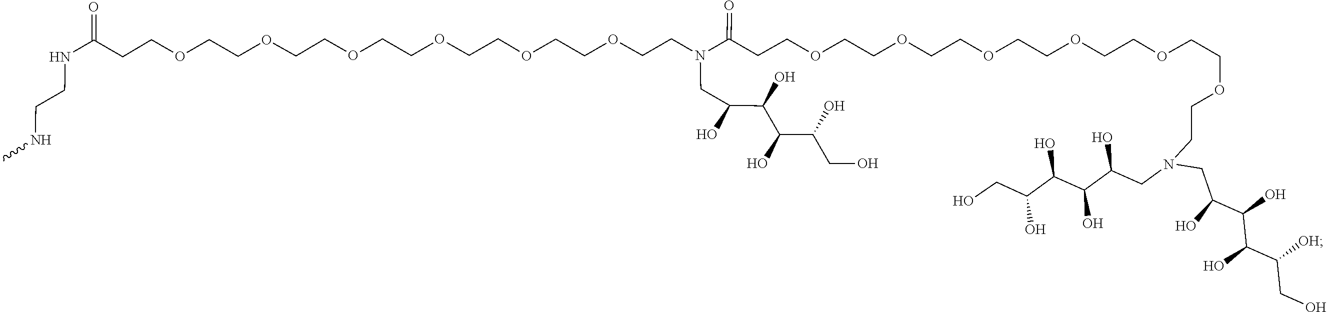 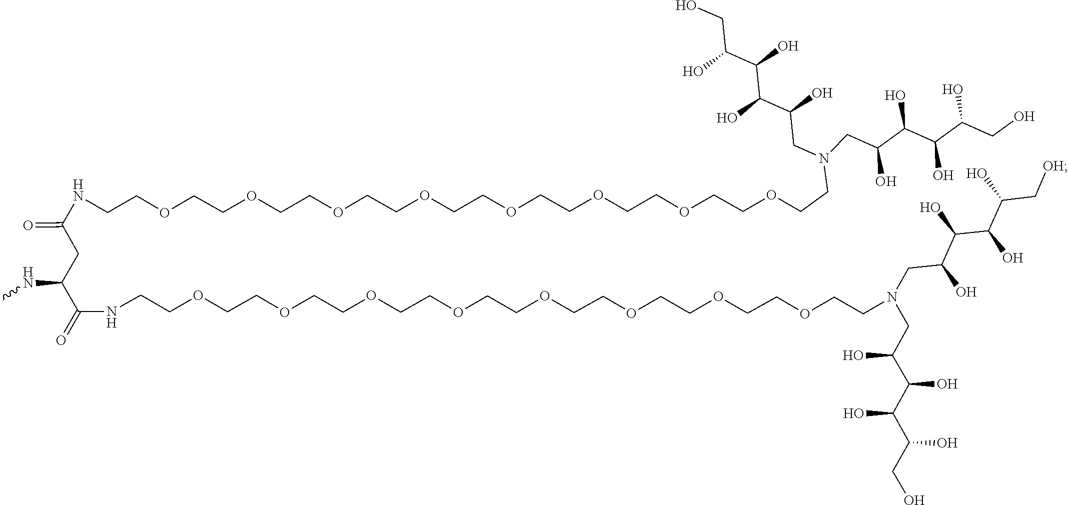 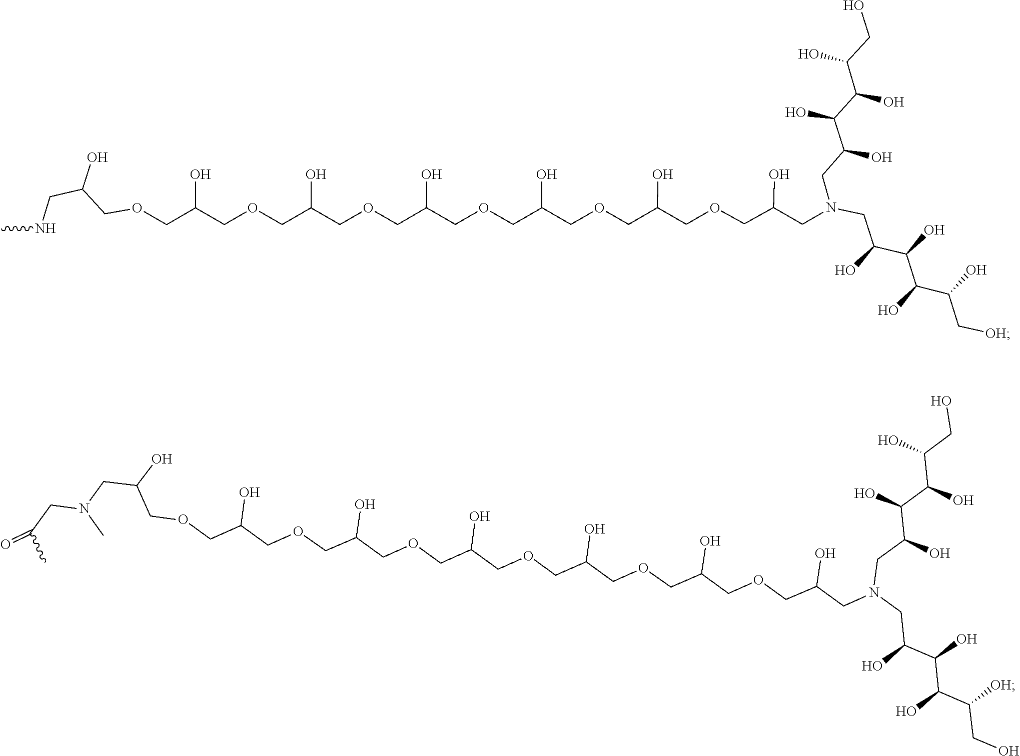 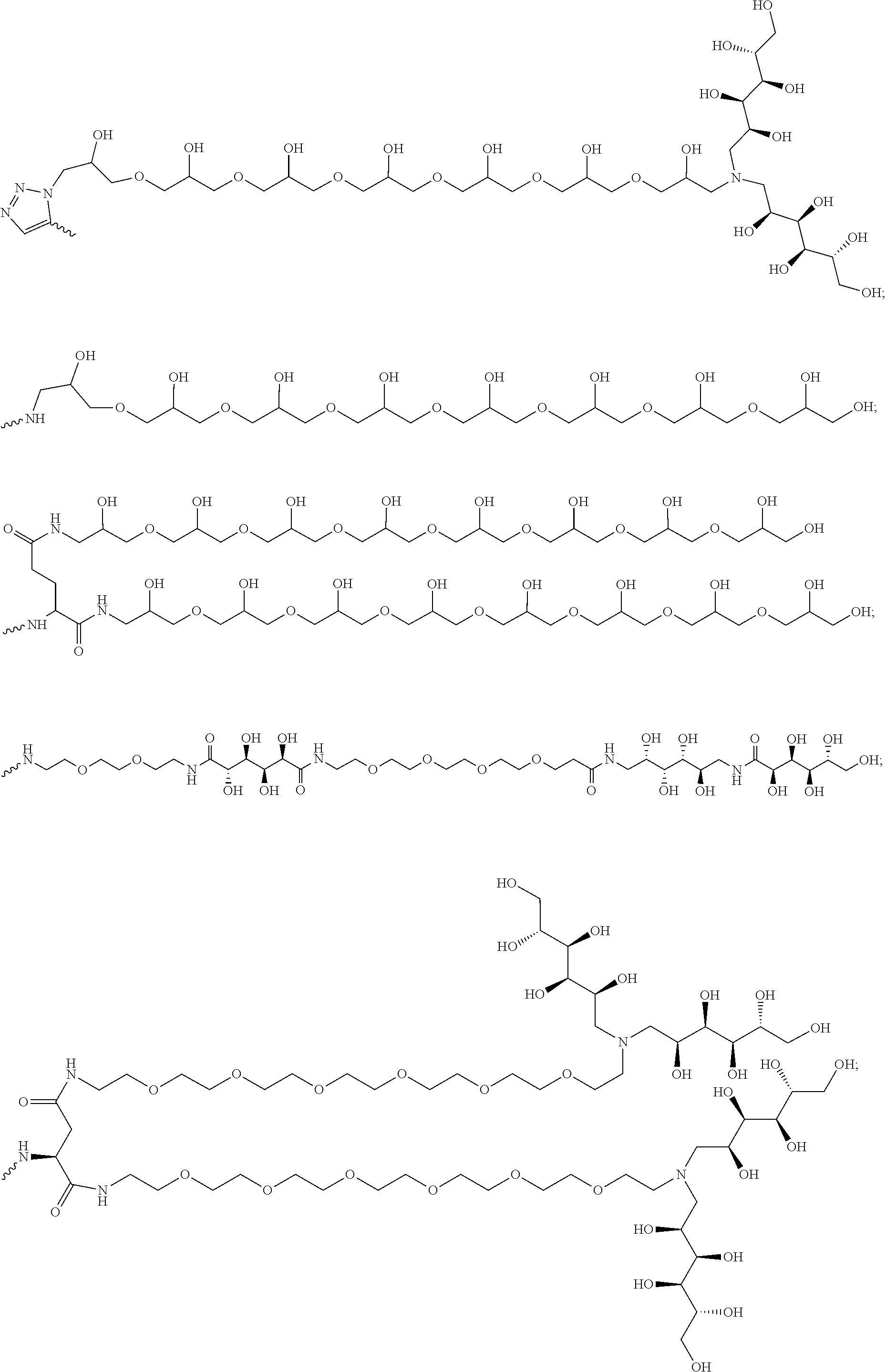 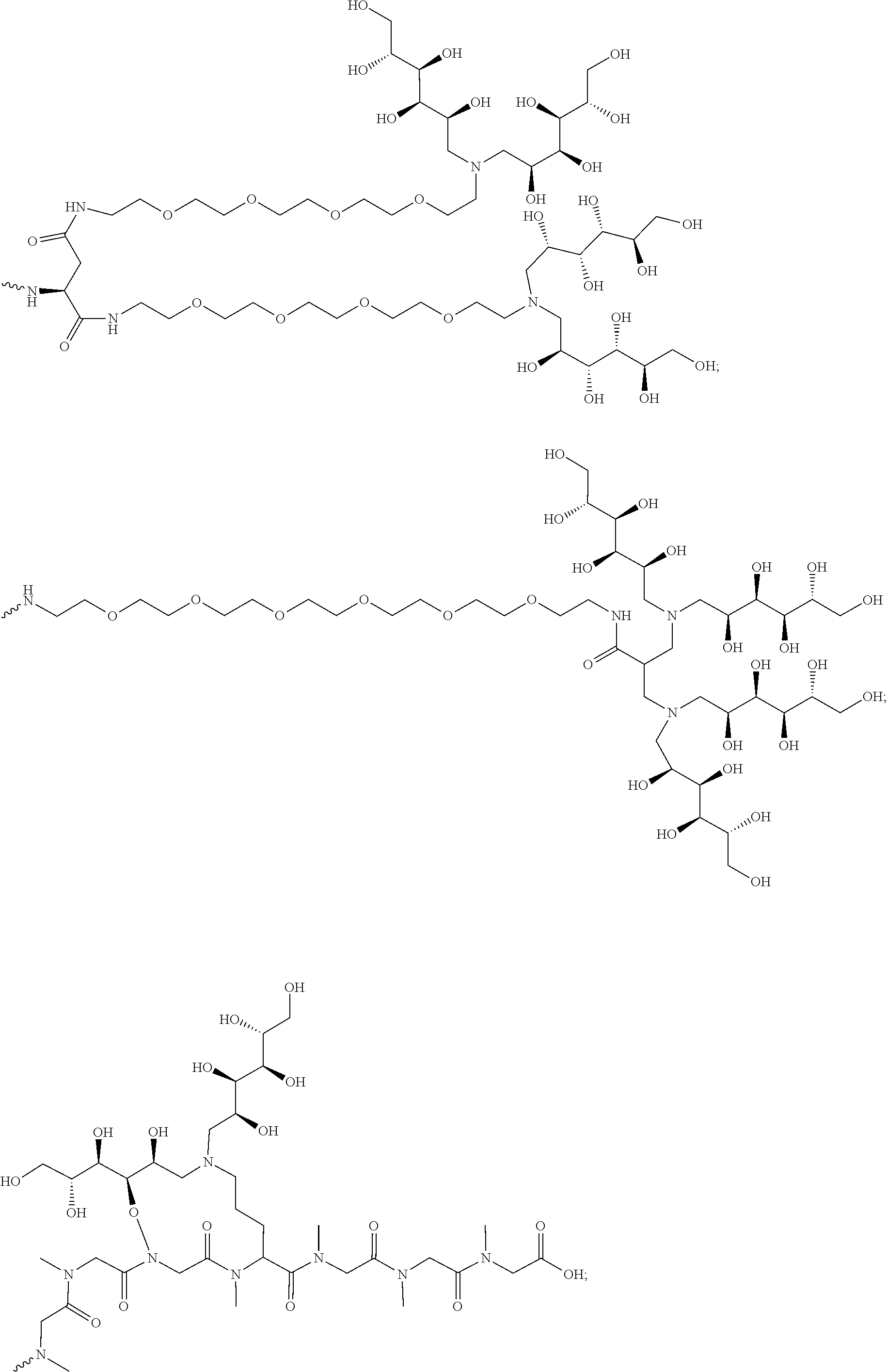 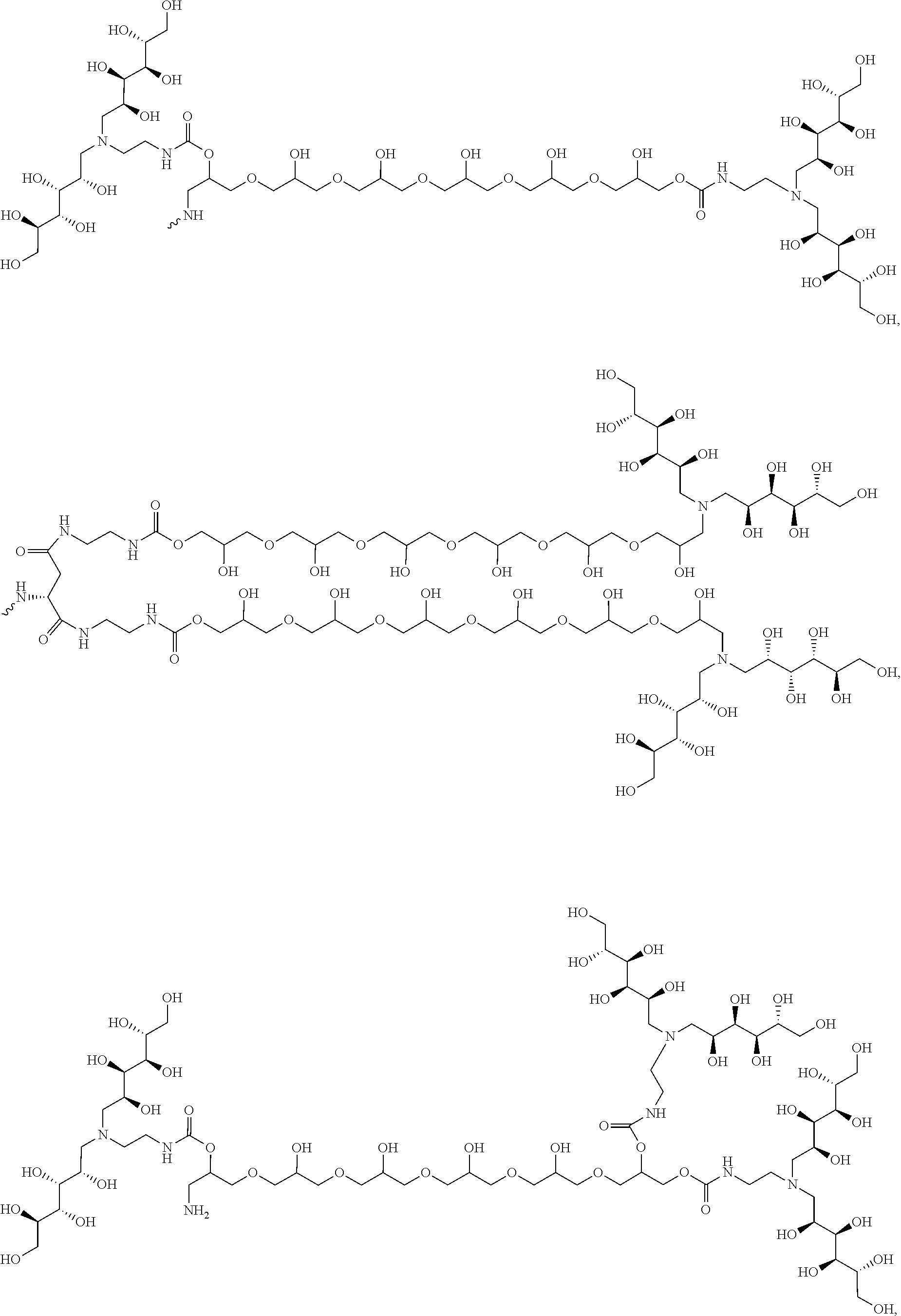 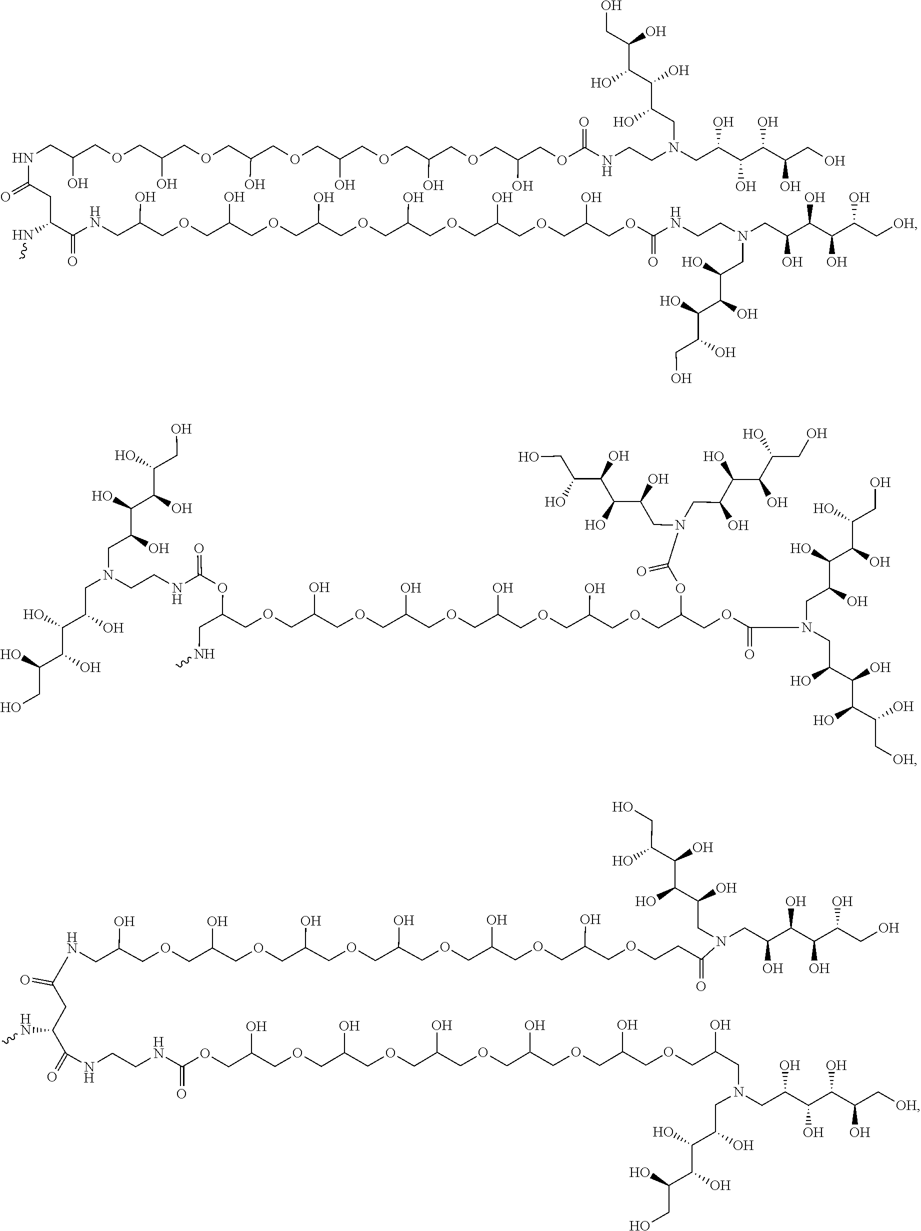 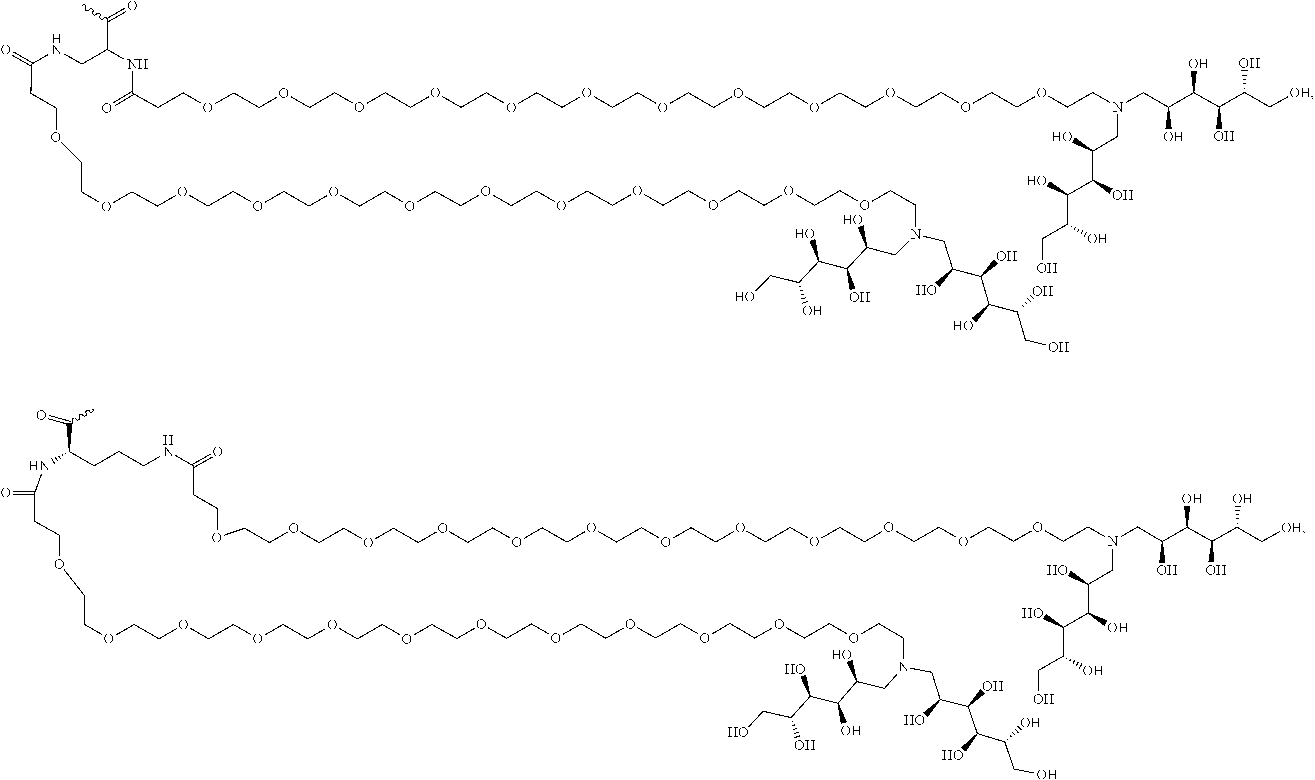 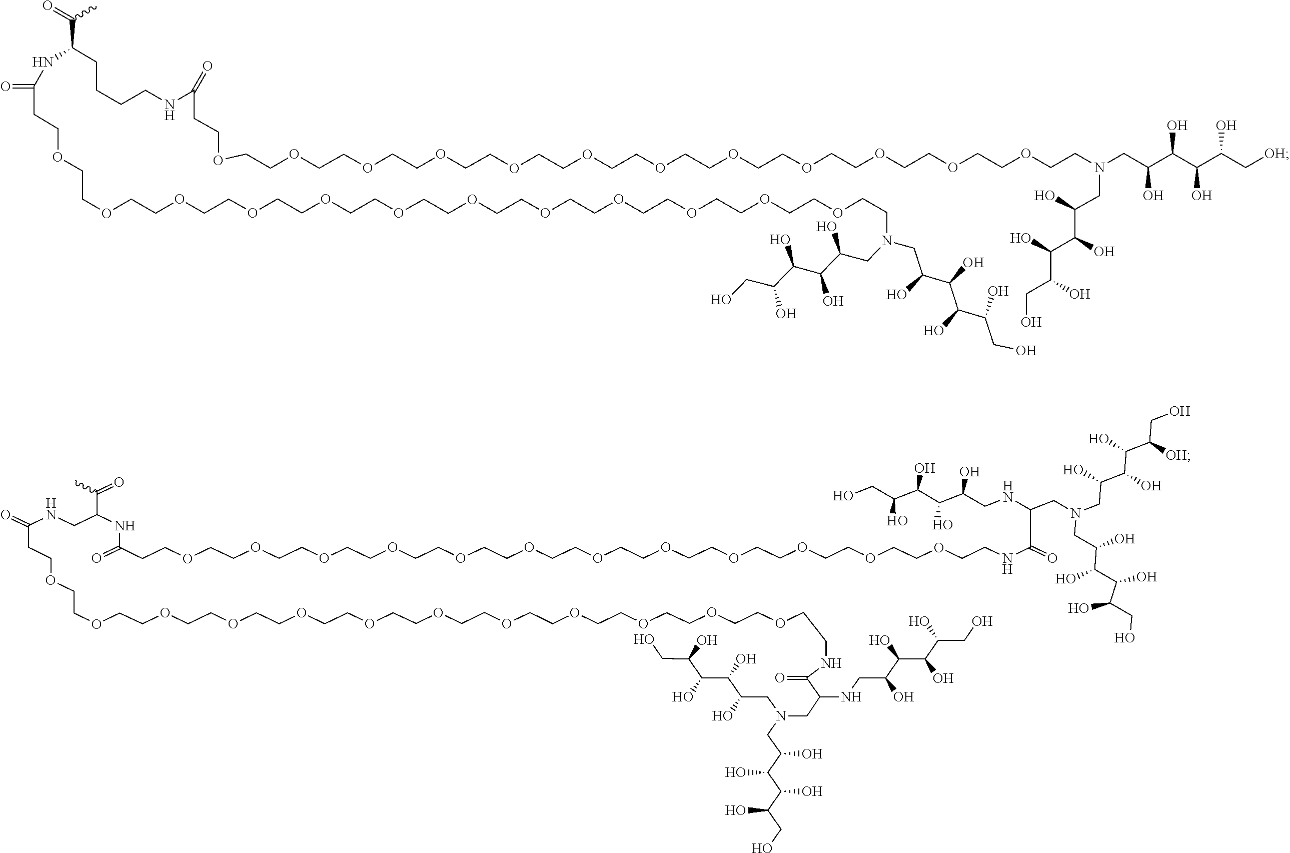 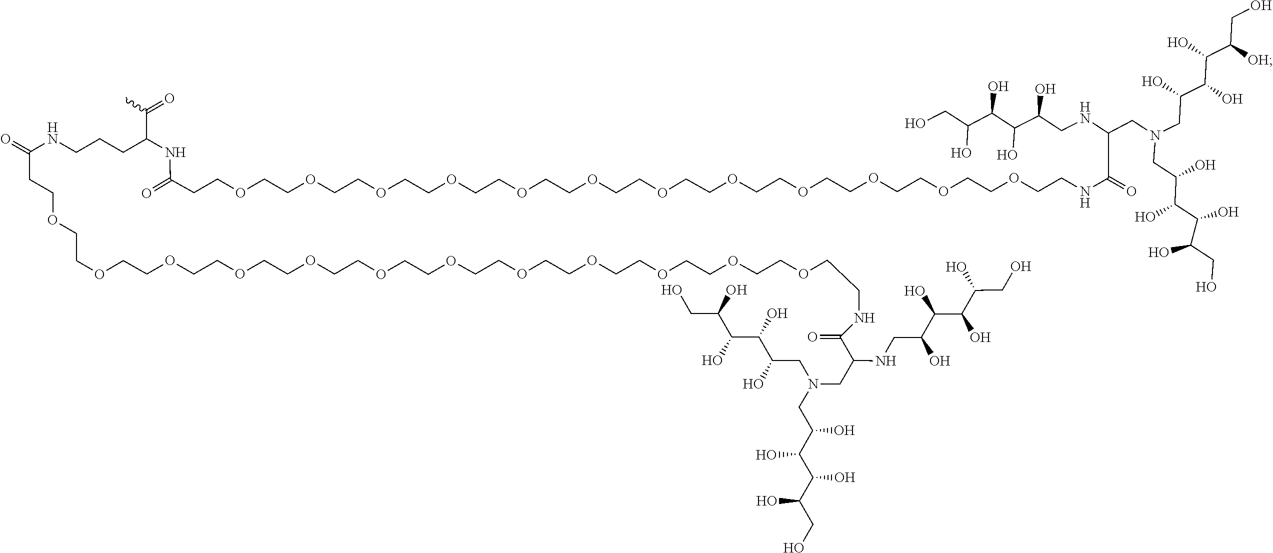 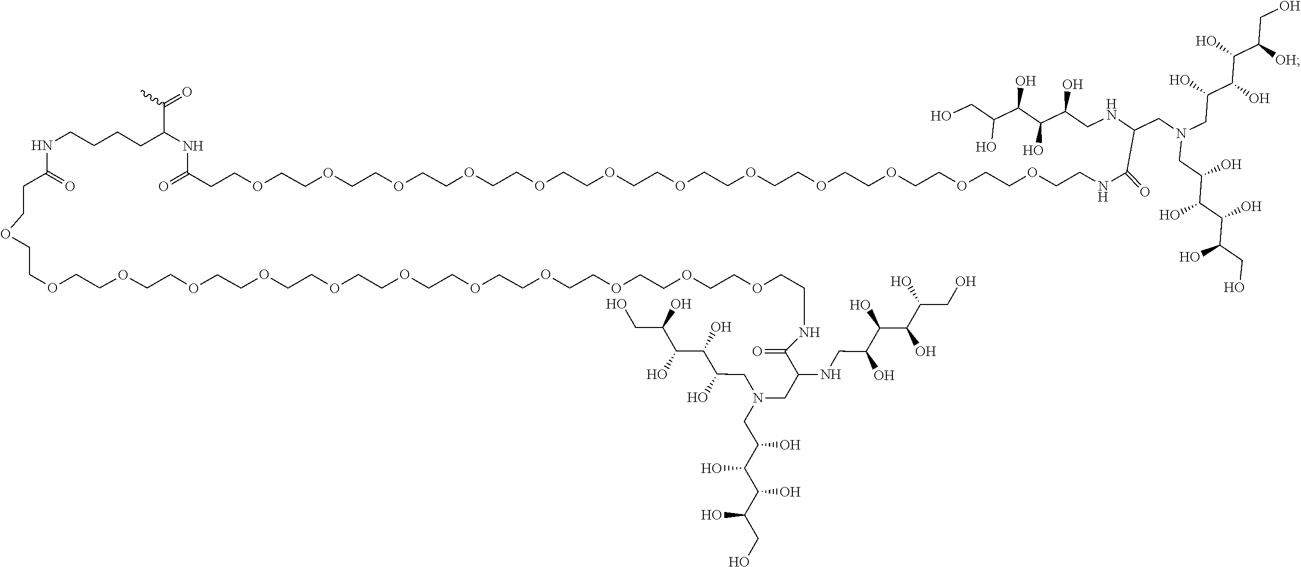 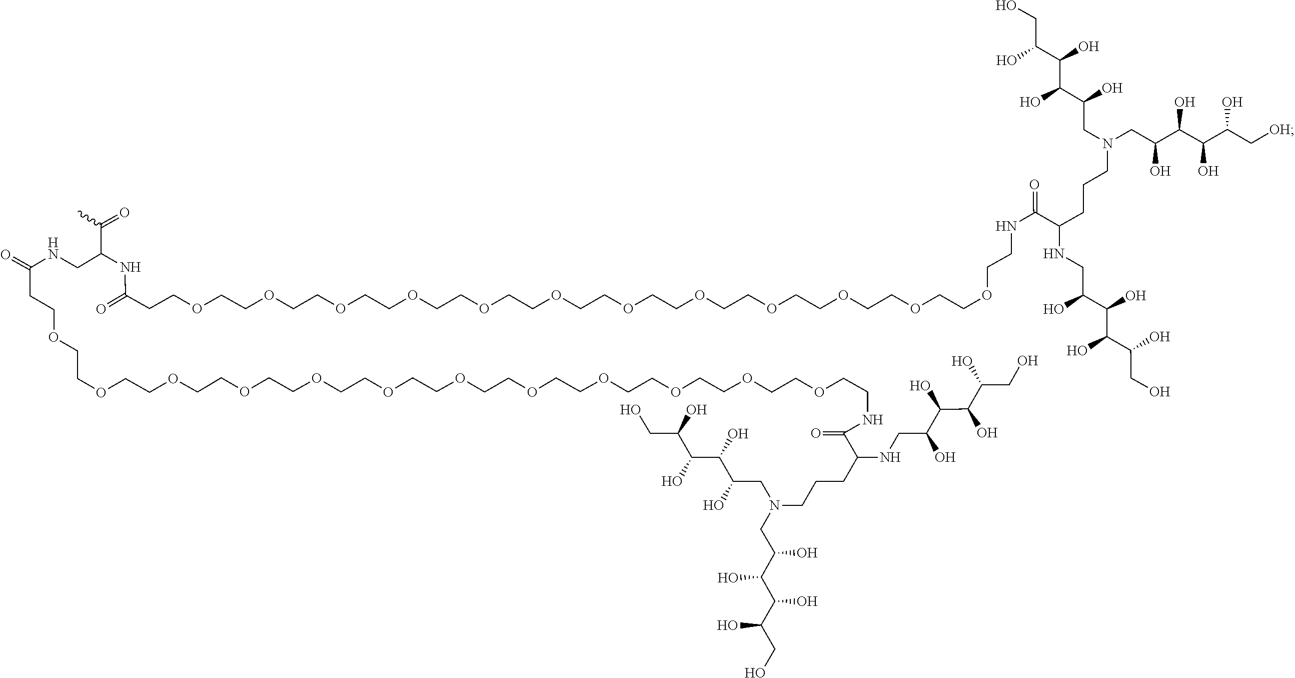 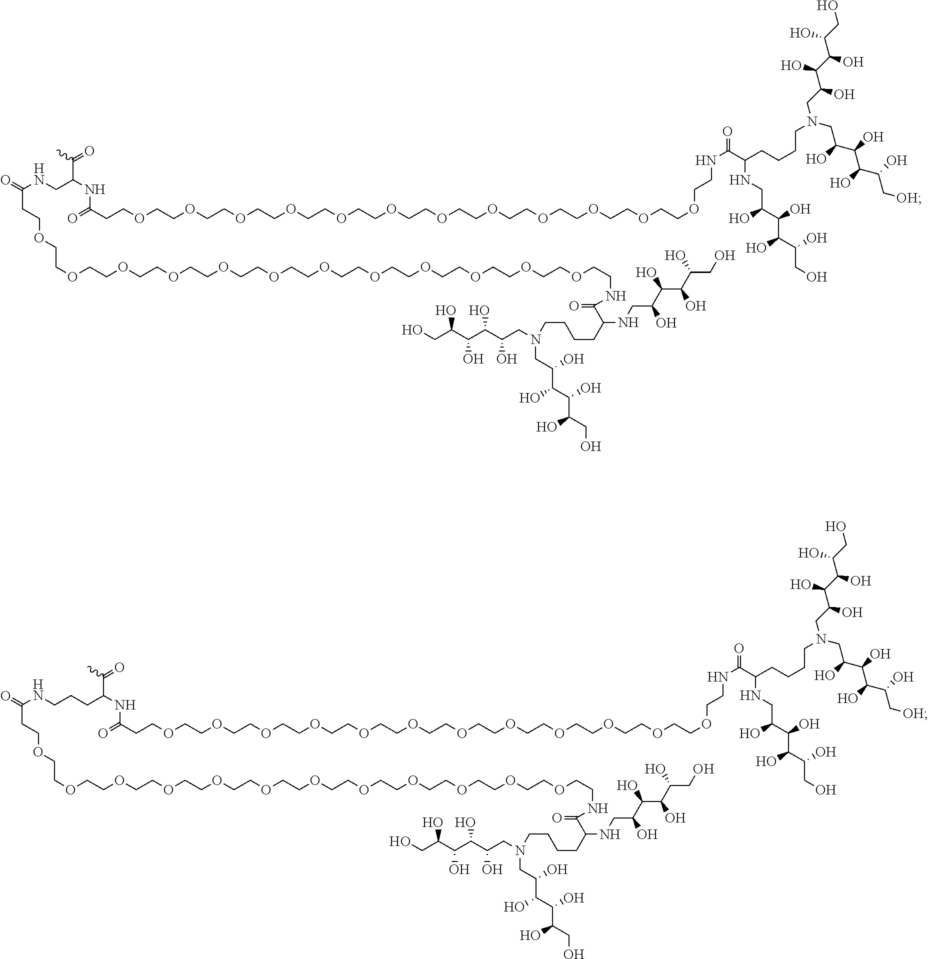 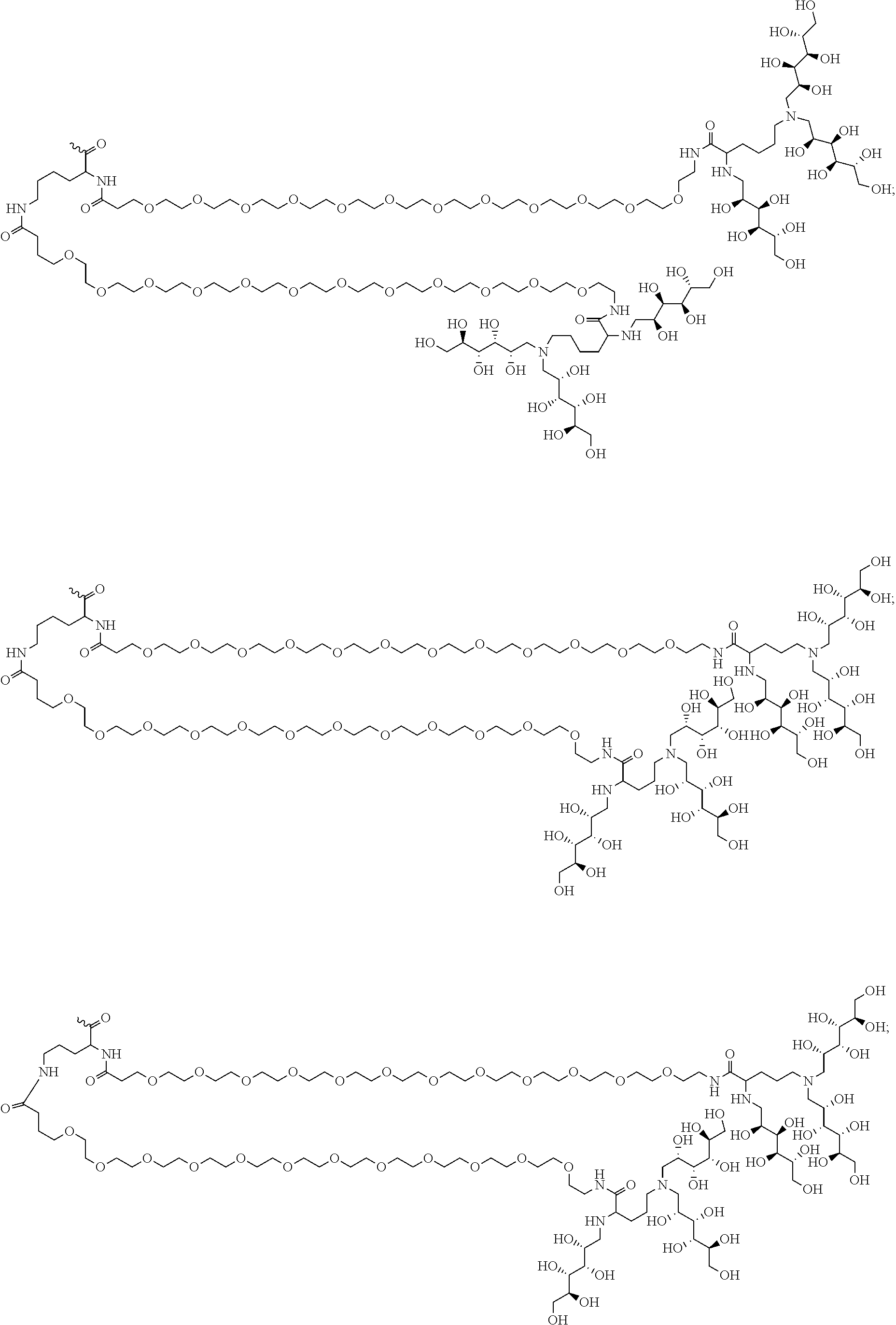 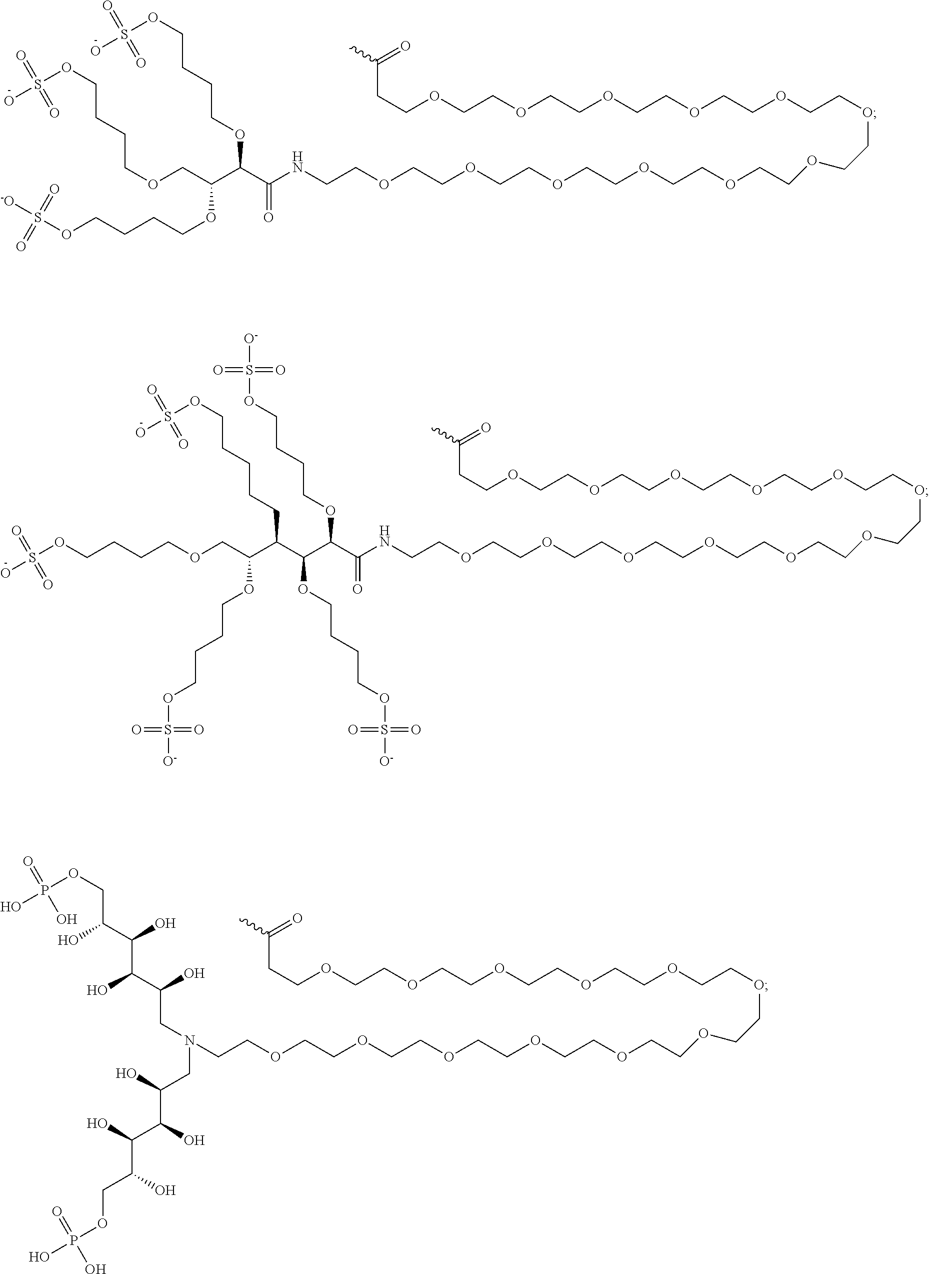 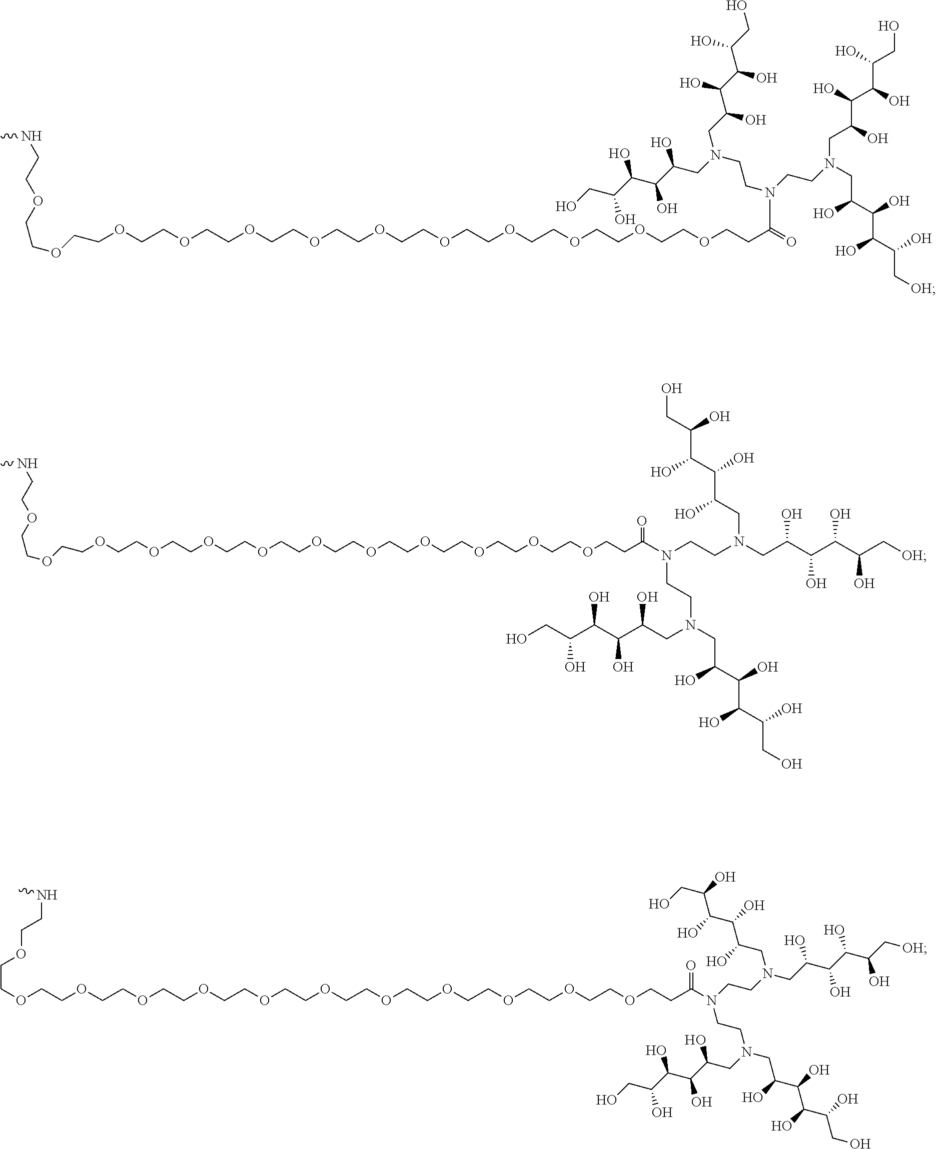 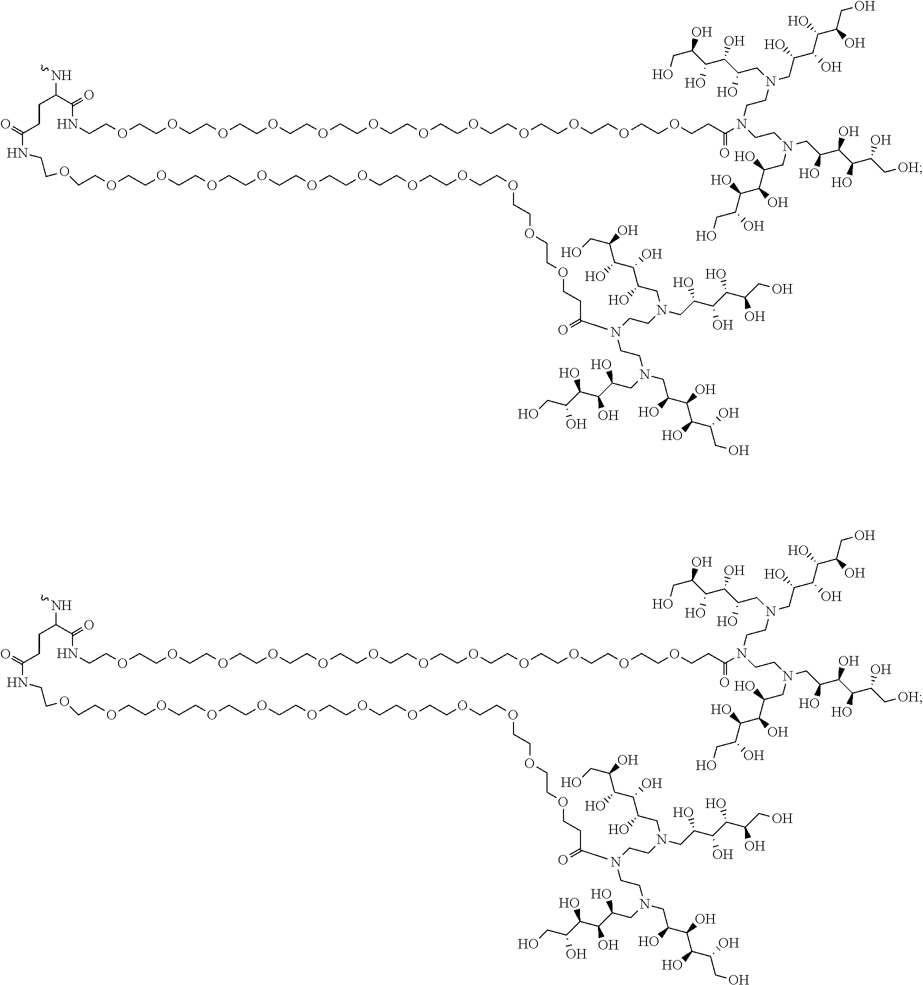 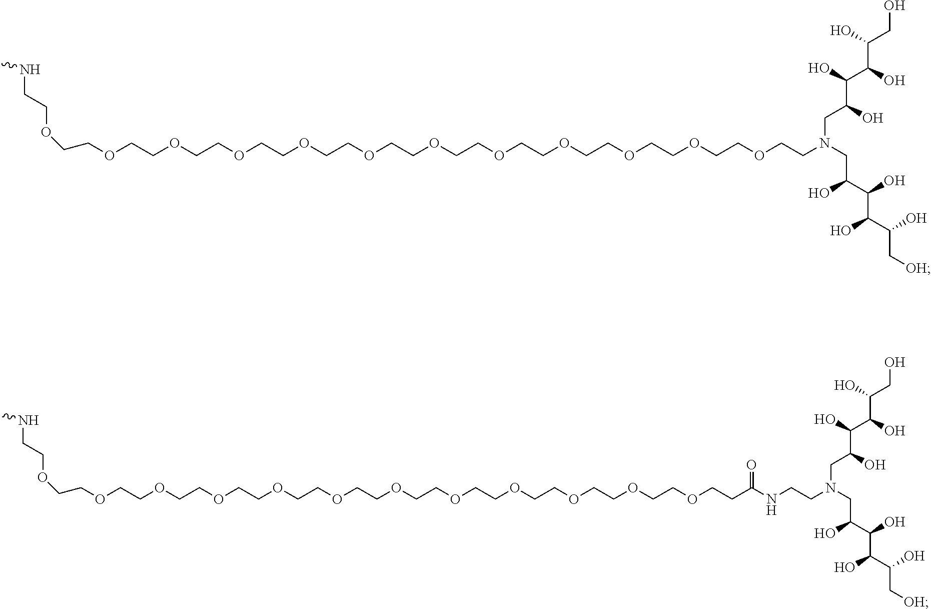 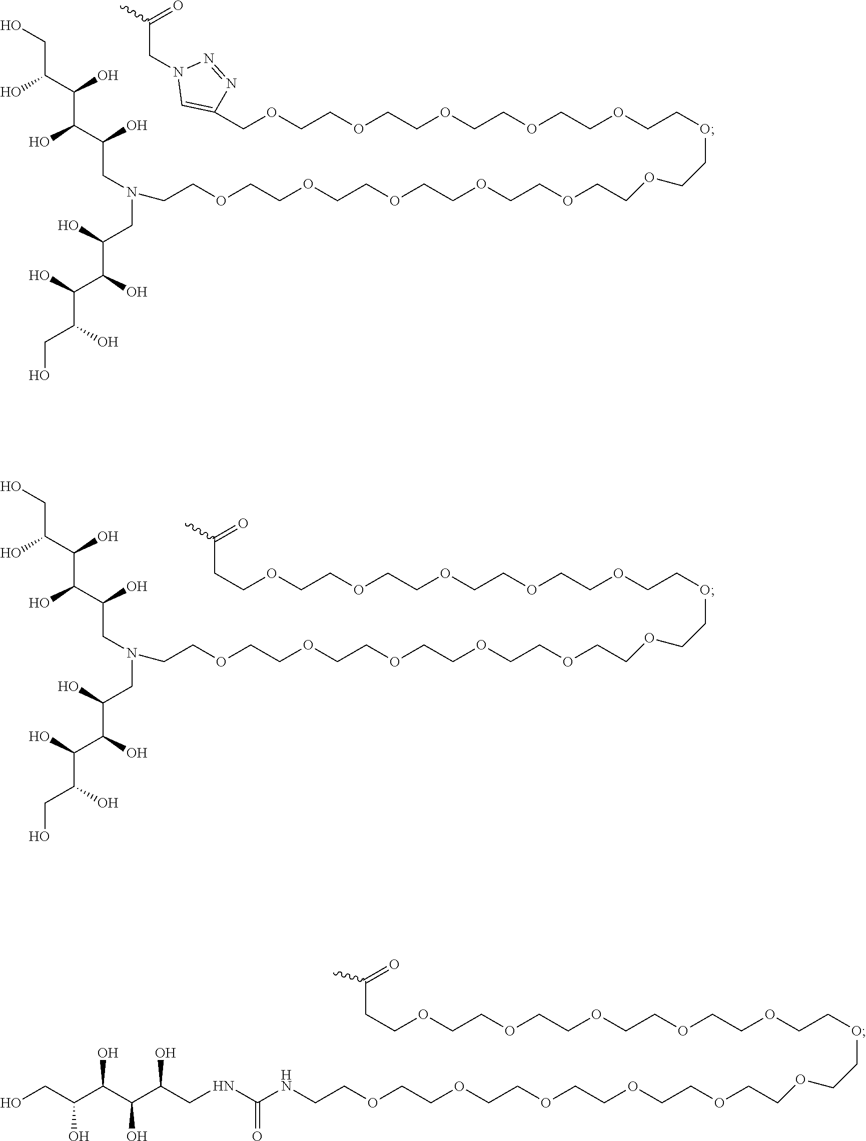 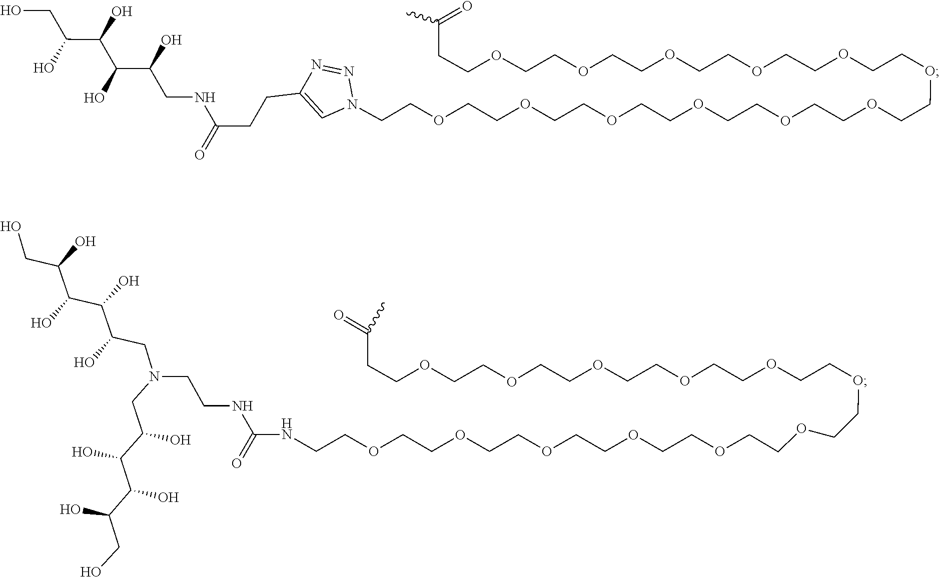 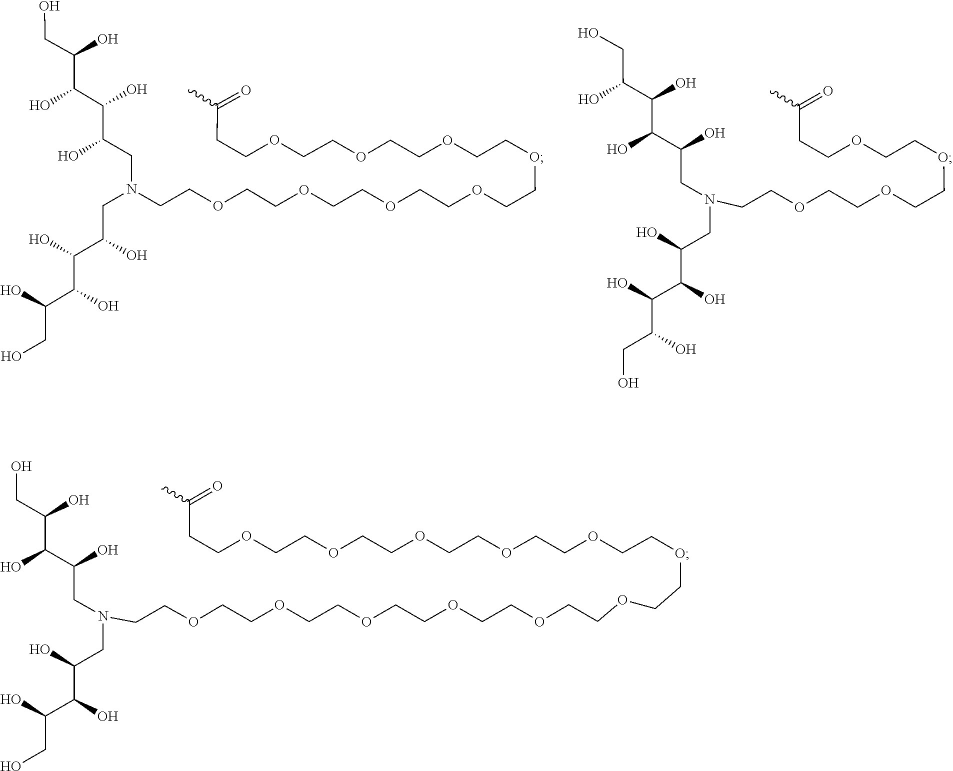 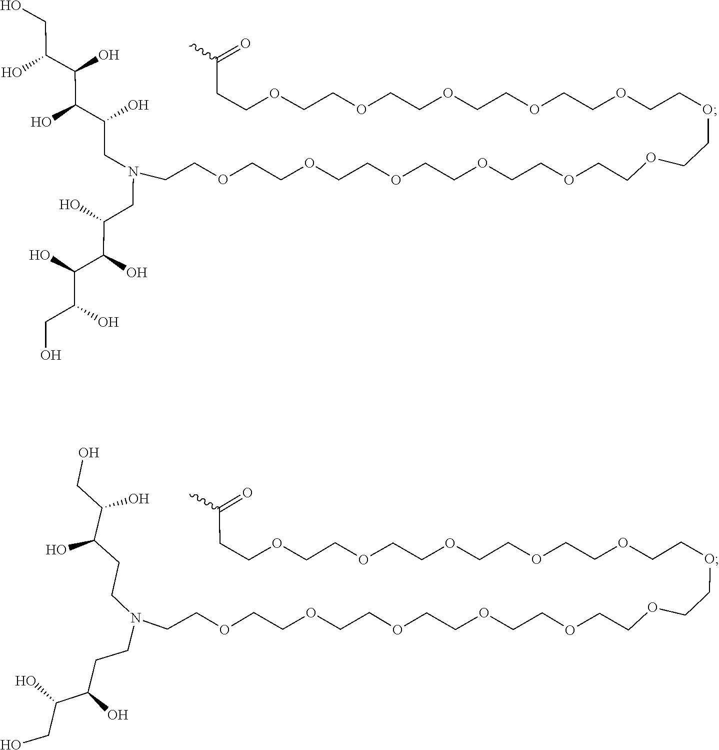 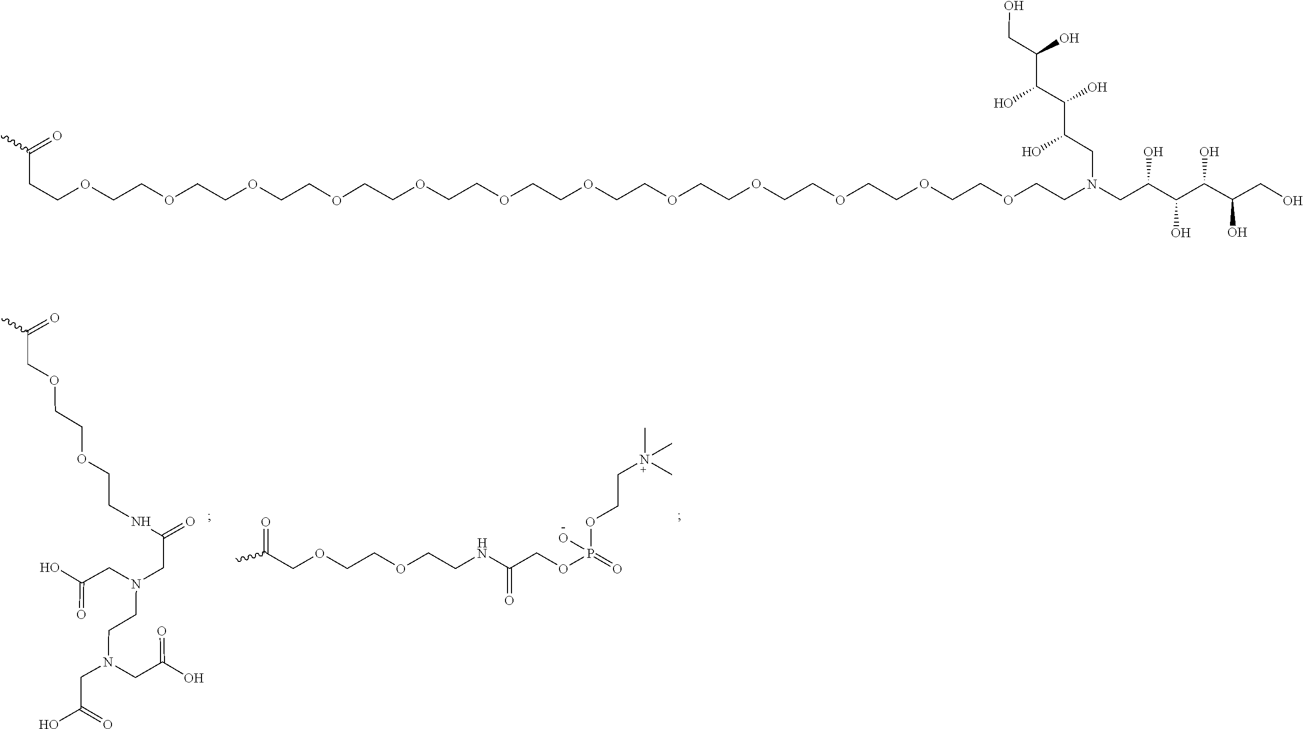 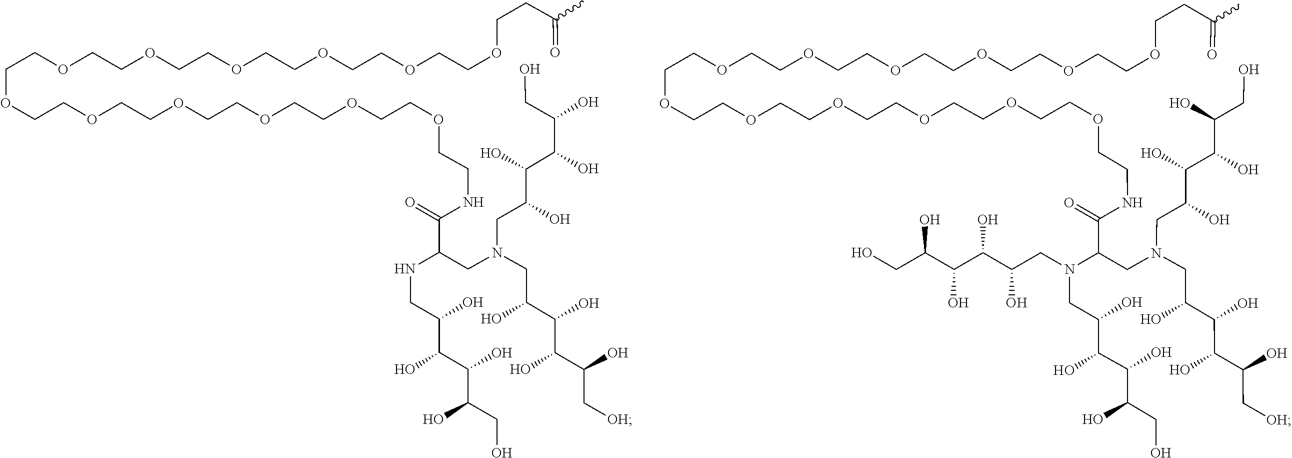 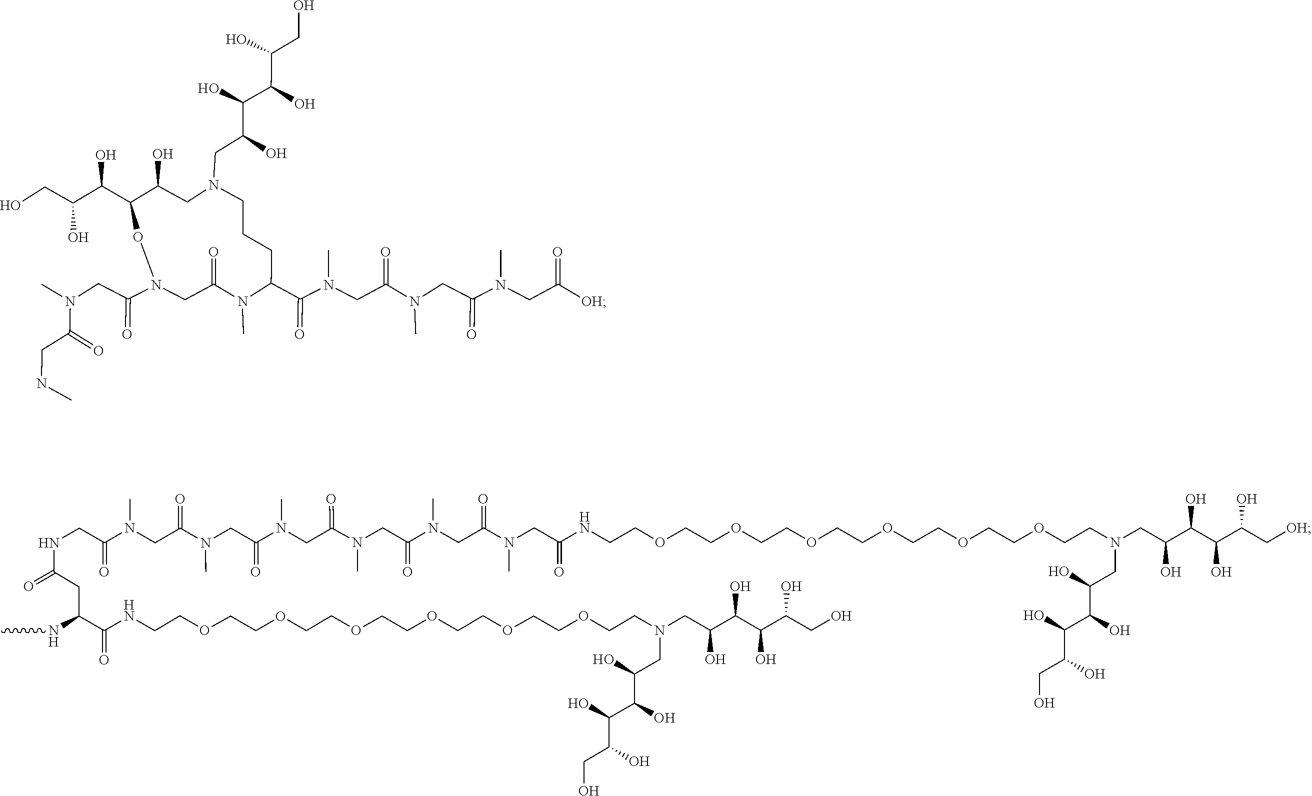 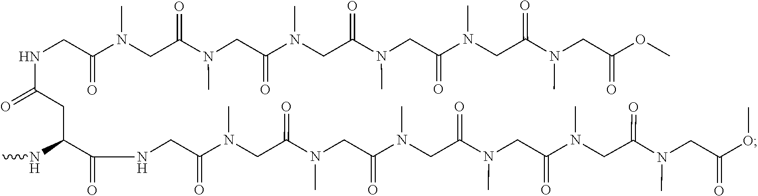 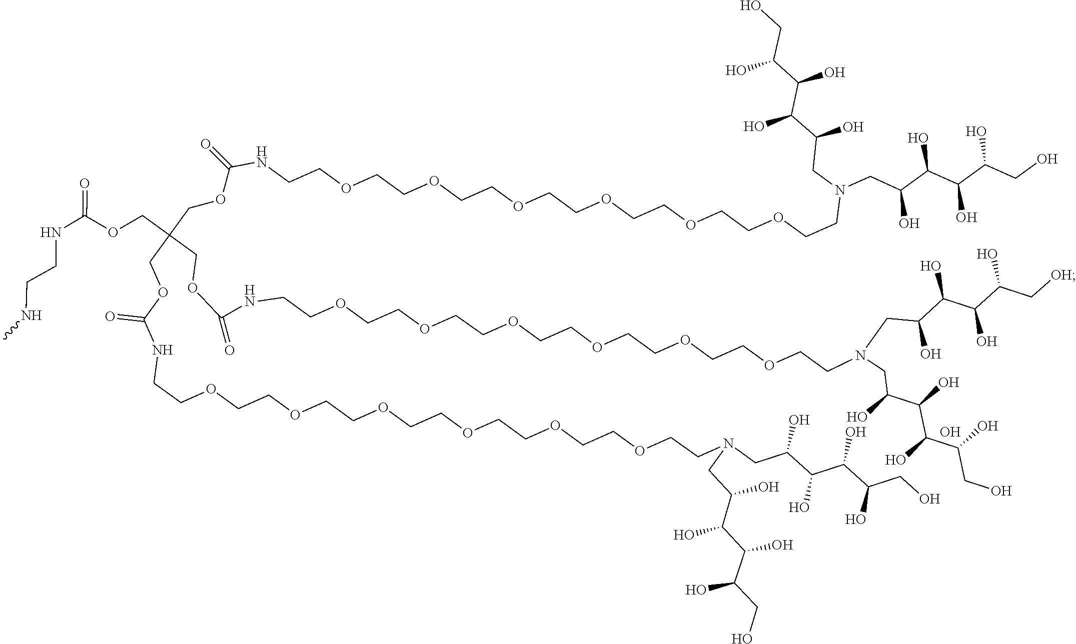 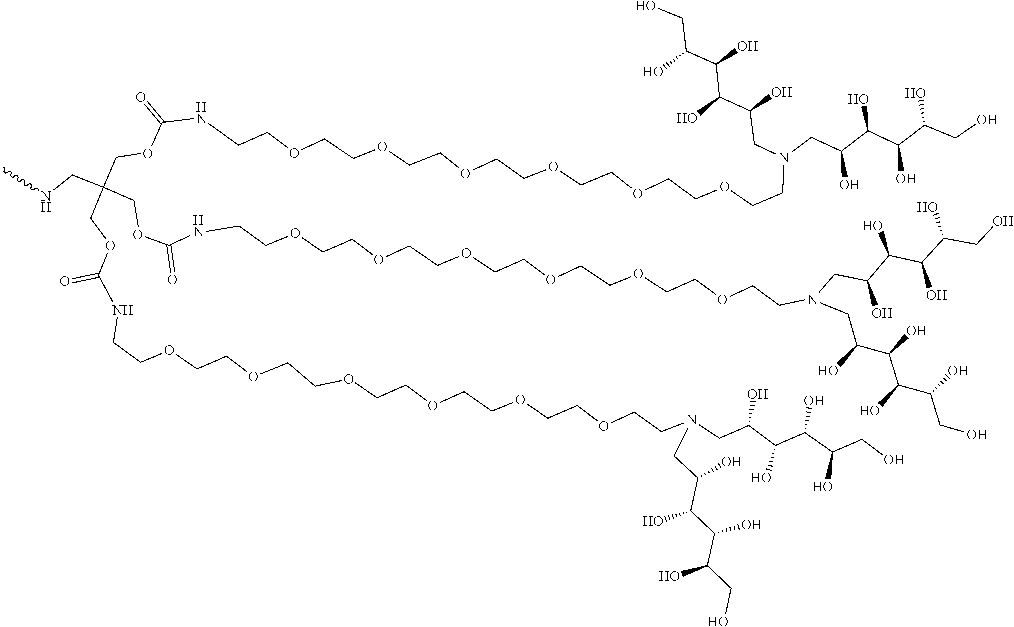 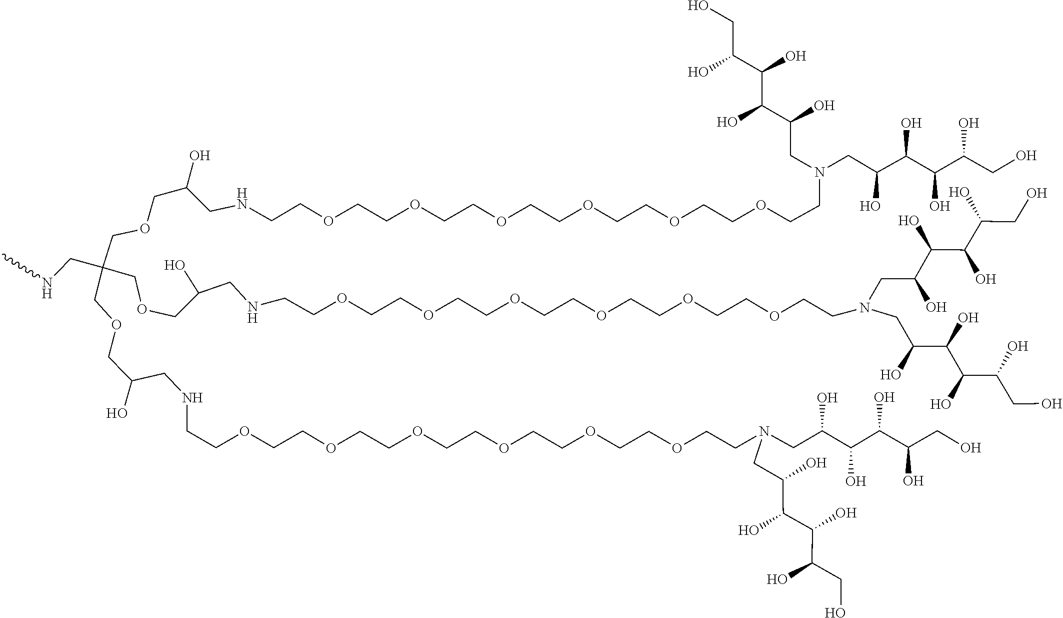 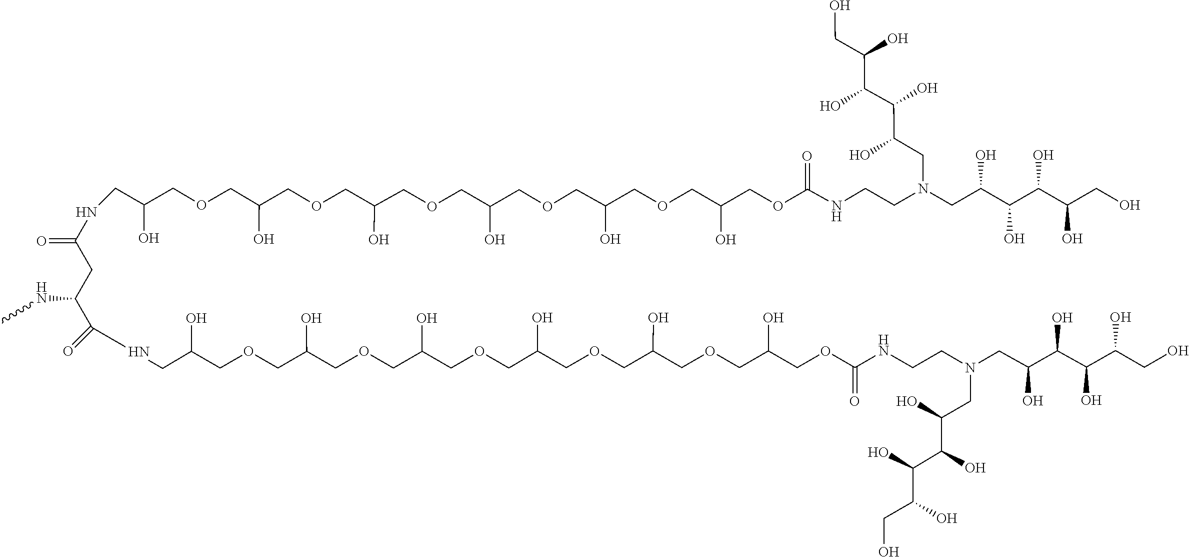 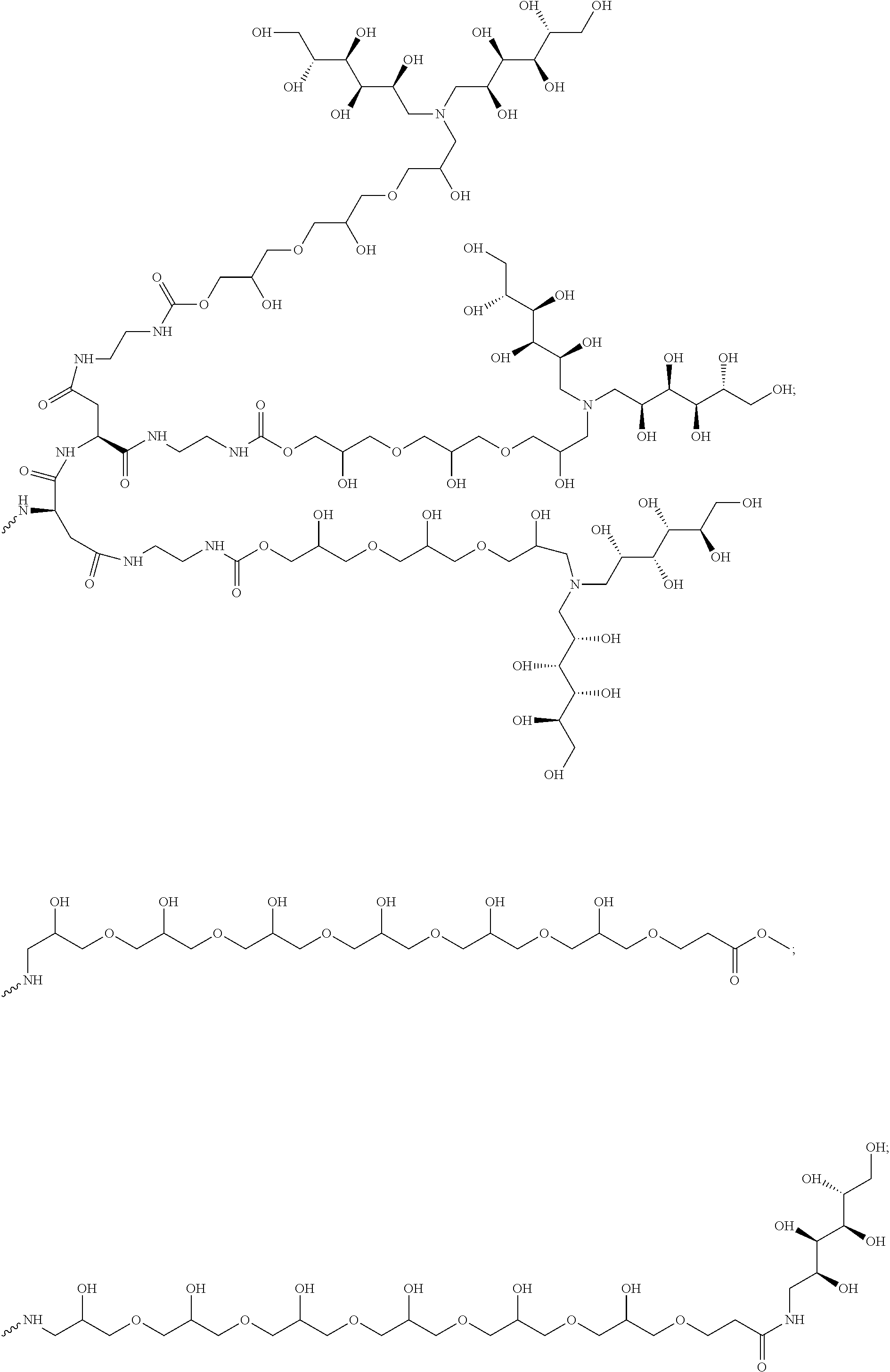 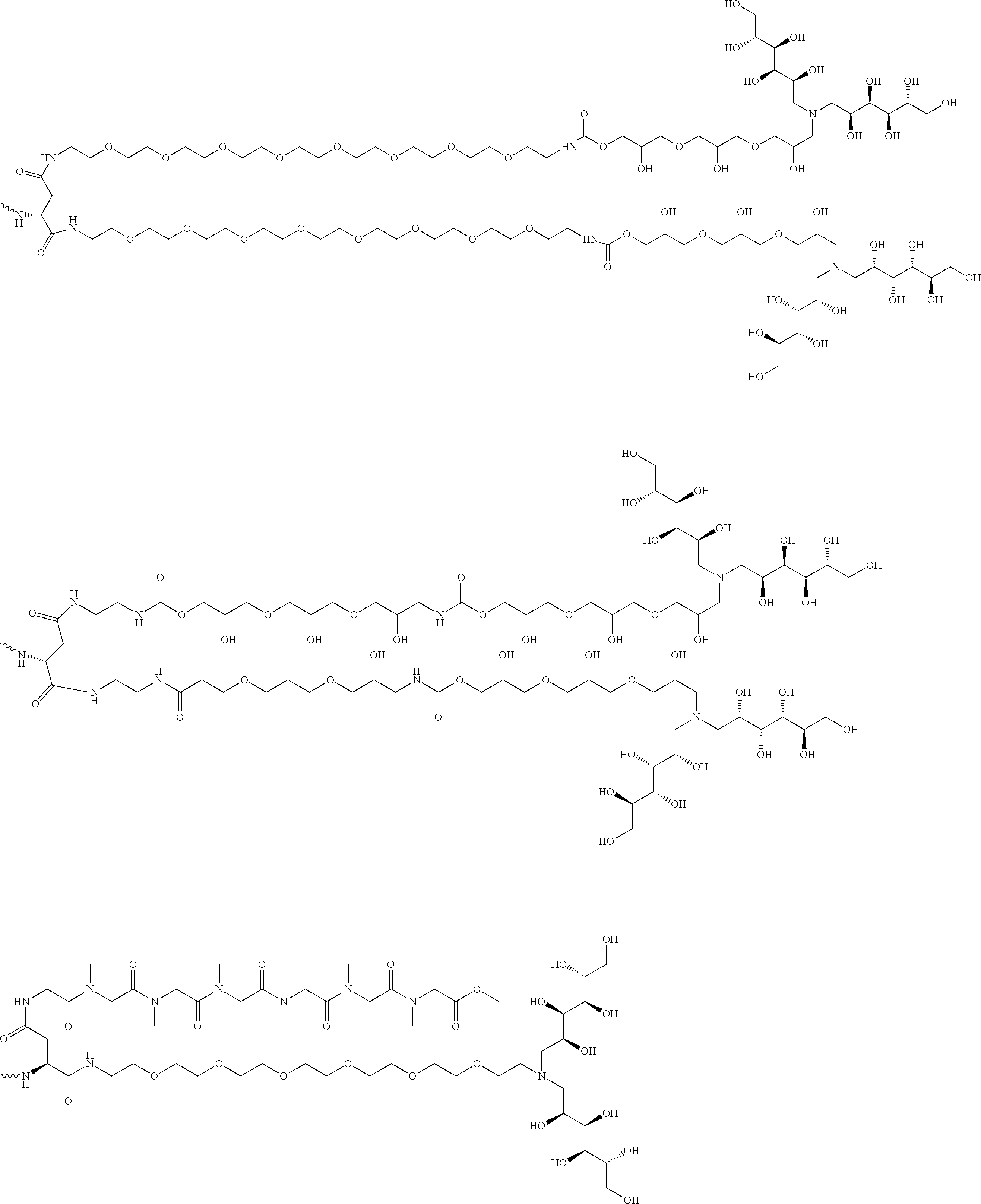 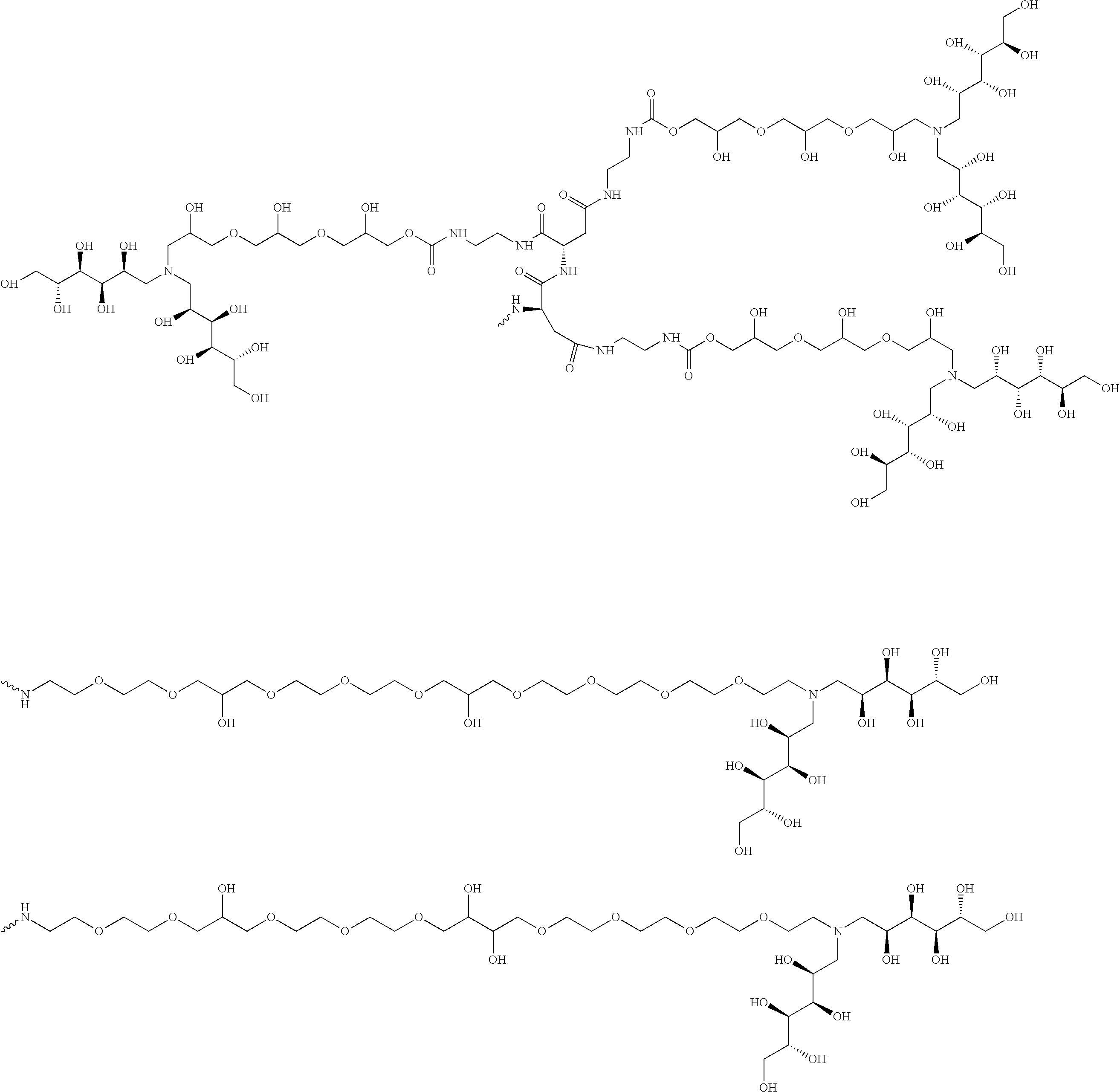 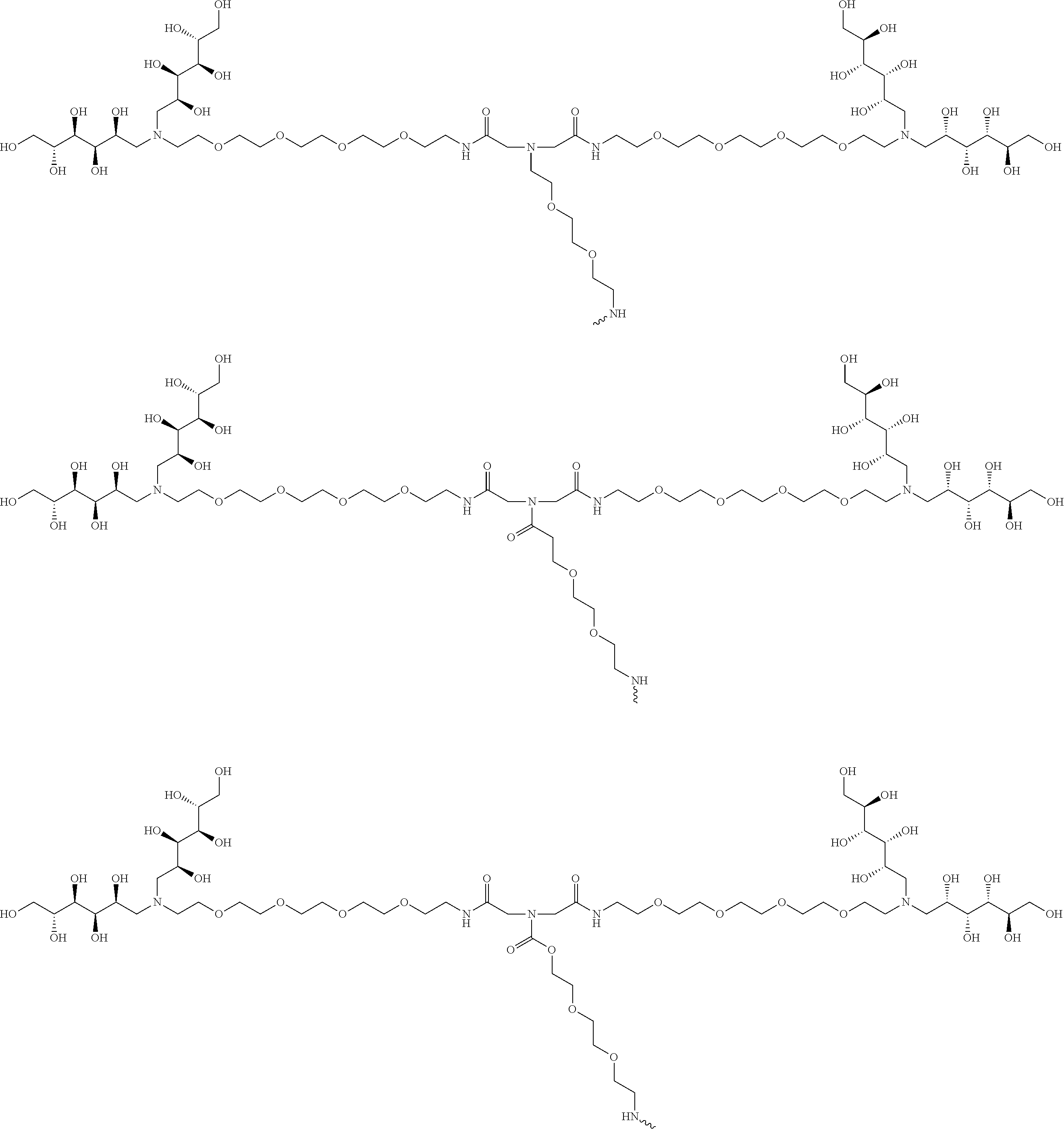 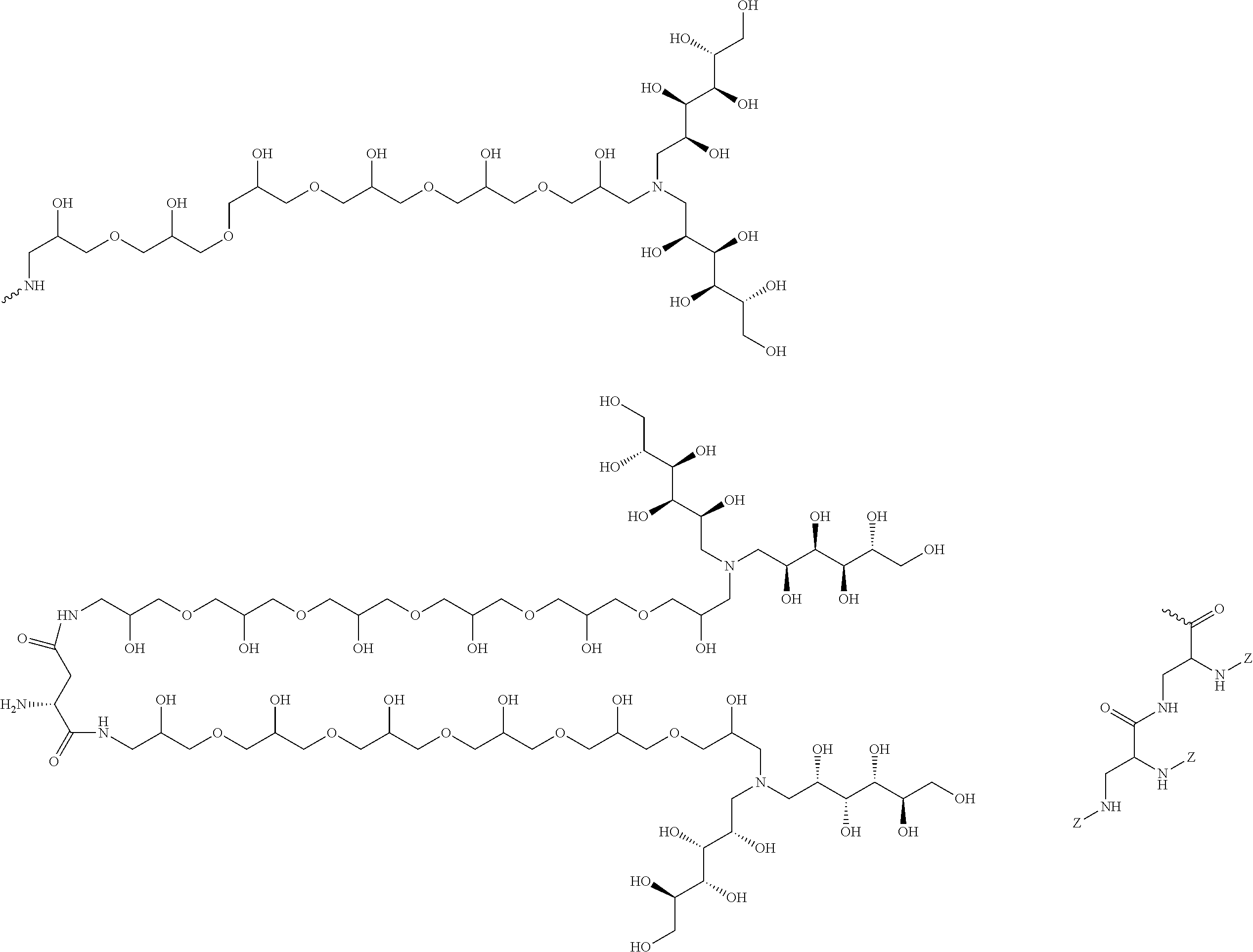
and
wherein each Z is attached at * and is individually selected from: 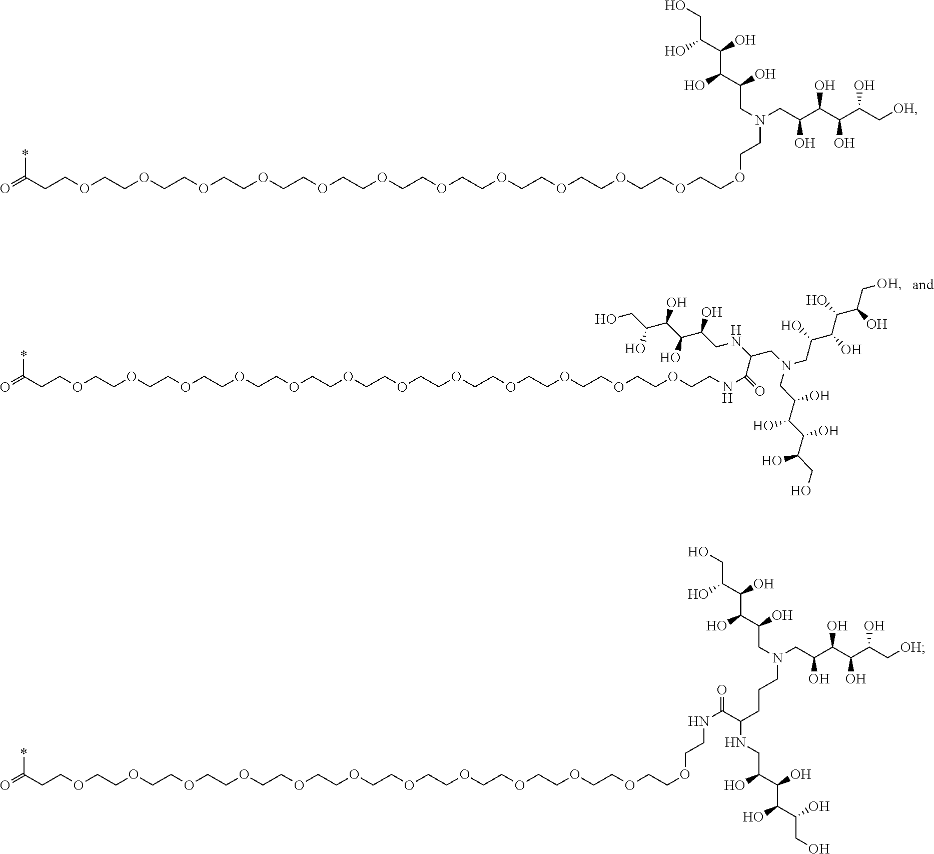 [SMILES: *C(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; *C(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO ; *C(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]
wherein [symbol] each is an attachment to site β or to site R^{b}, or to the enzyme-cleavable group.

55. The conjugate of claim 30, comprising:
(a) a polar group including the polymer unit and a sugar unit,
(b) a polar group including at least two polymer units;
(c) a polar group including the polymer unit and a carboxyl unit:
(d) at least two polar groups:
(e) a polar group including the polymer unit, the sugar unit and the carboxyl unit: or
(f) a polar group including at least two polymer units, at least one sugar unit and at least one carboxyl unit.

56.-60. (canceled)

61. The conjugate of claim 30, wherein the enzyme-cleavable group comprises at least two of the amino acid units.

62. The conjugate of claim 30, comprising at least one polar group being attached to the enzyme-cleavable group.

63. The conjugate of claim 30, having one of the following structures: 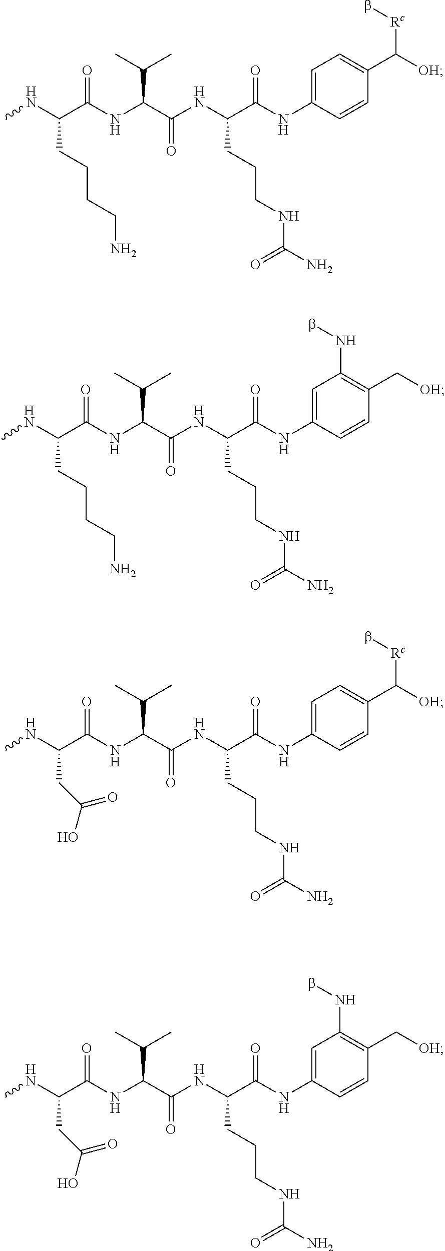 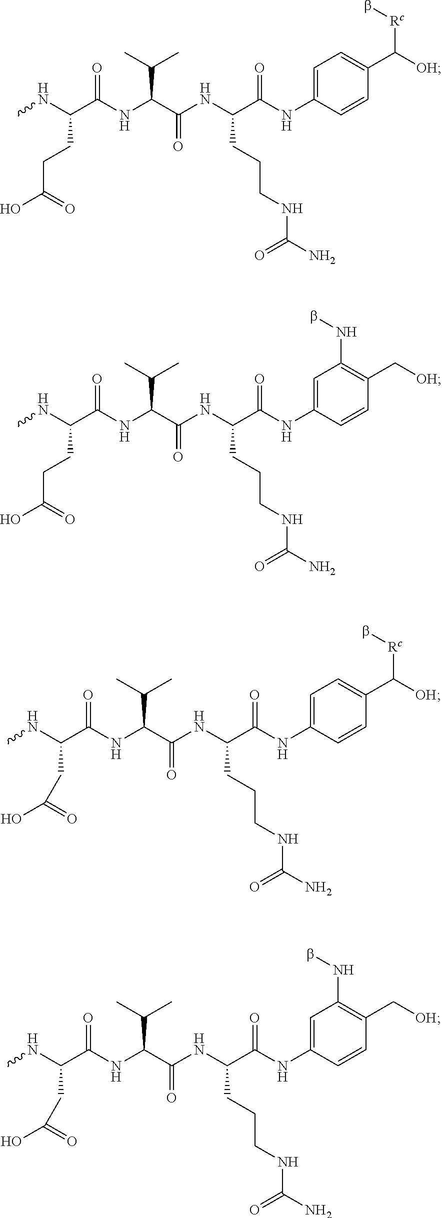 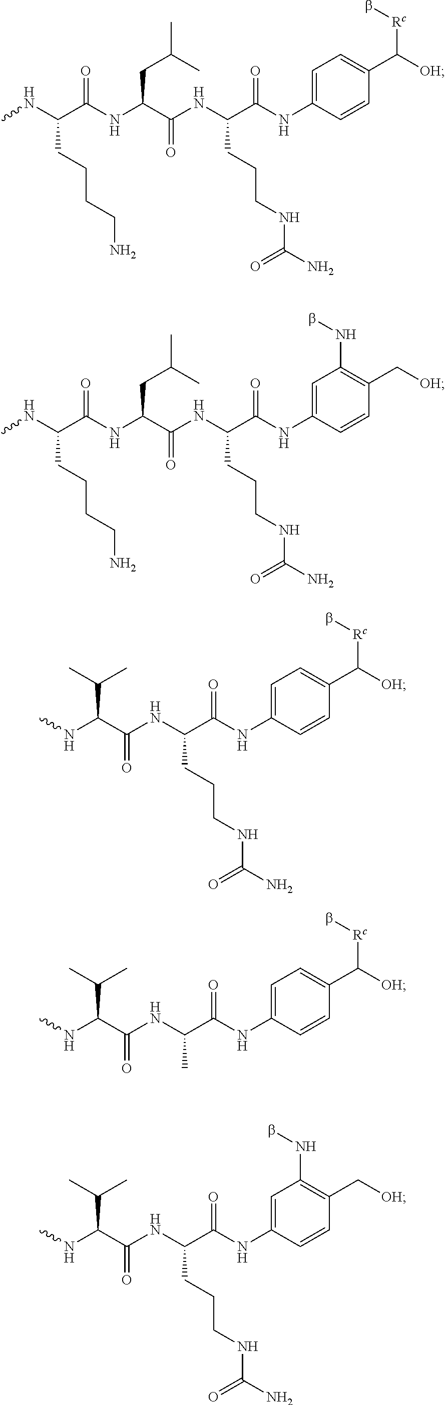 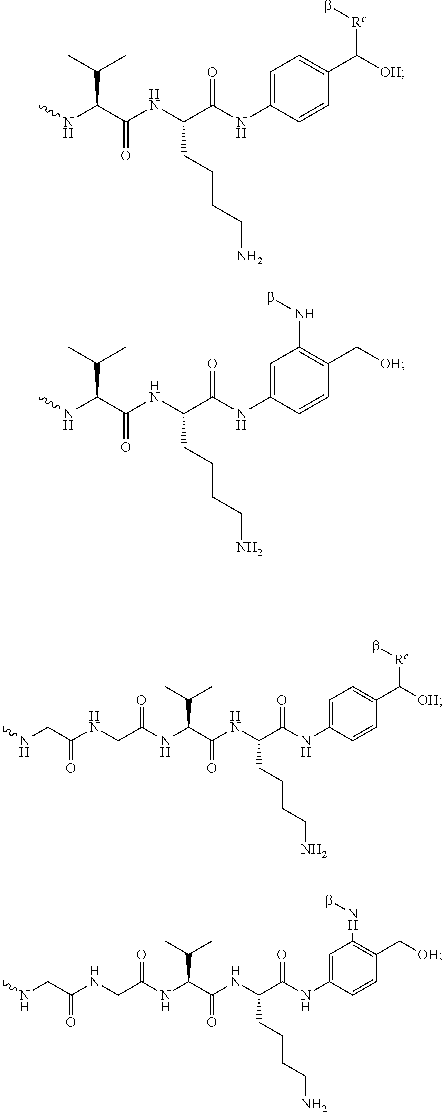 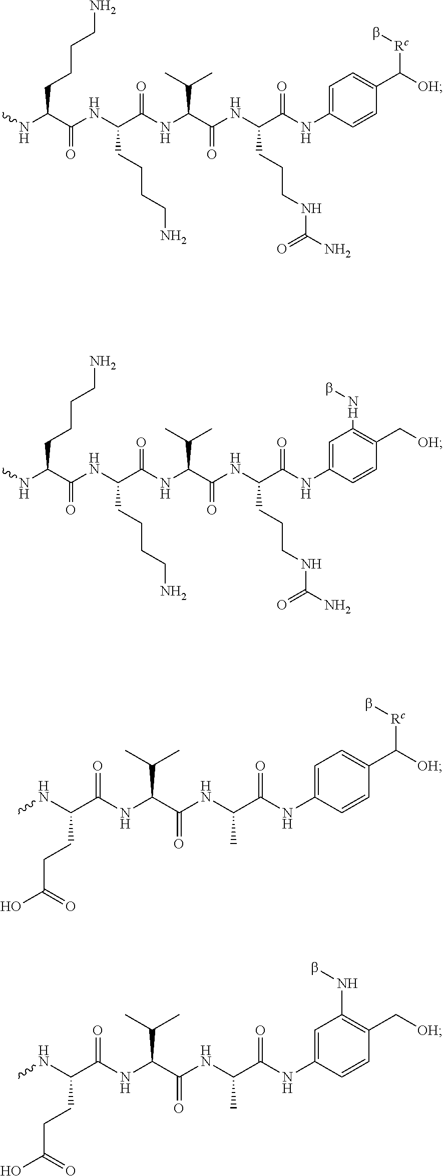 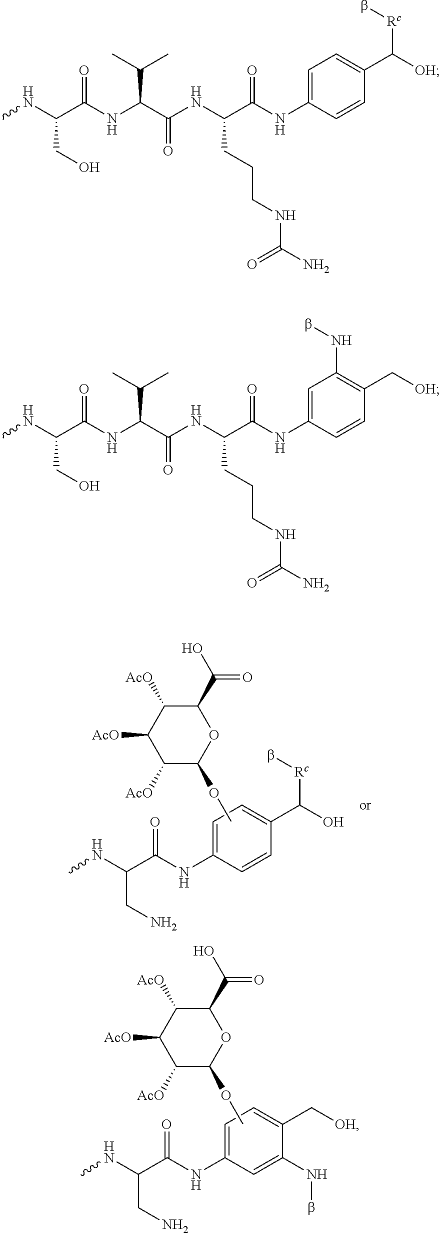
wherein
Rc is a bond or C₁₋₆ alkylene;
the wavy line on the amino group indicates an attachment site for a stretcher group or, prior to attachment to the stretcher group, indicates H;
β— is the attachment site to the at least one polar group; and
the benzylic H on the benzylic OH is optionally replaced with a bond to one of the drug units or to the linking group attached to at least one of the drug units.

64. The conjugate of claim 30, wherein the enzyme-cleavable group comprises a peptide that is cleavable by an intracellular protease.

65. The conjugate of claim 64, wherein the intracellular protease is Cathepsin B.

66. The conjugate of claim 65, wherein the enzyme-cleavable group comprises a cleavable peptide including a valine-citrulline peptide, a valine-alanine peptide, a valine-lysine peptide, a phenylalanine-lysine peptide, or a glycine-glycine-phenylalanine-glycine peptide.

67. The conjugate of claim 64, comprising one of the following structures: 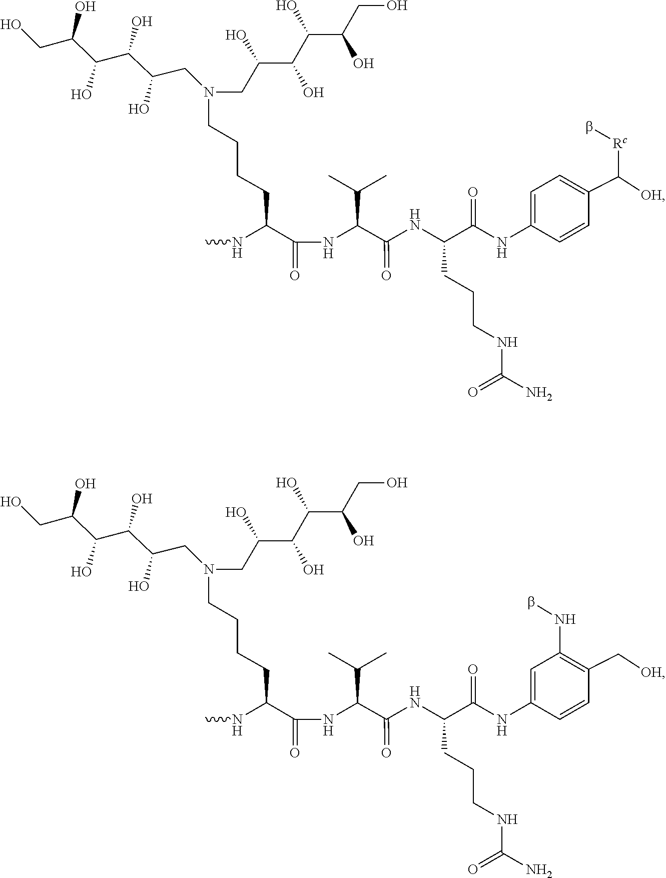 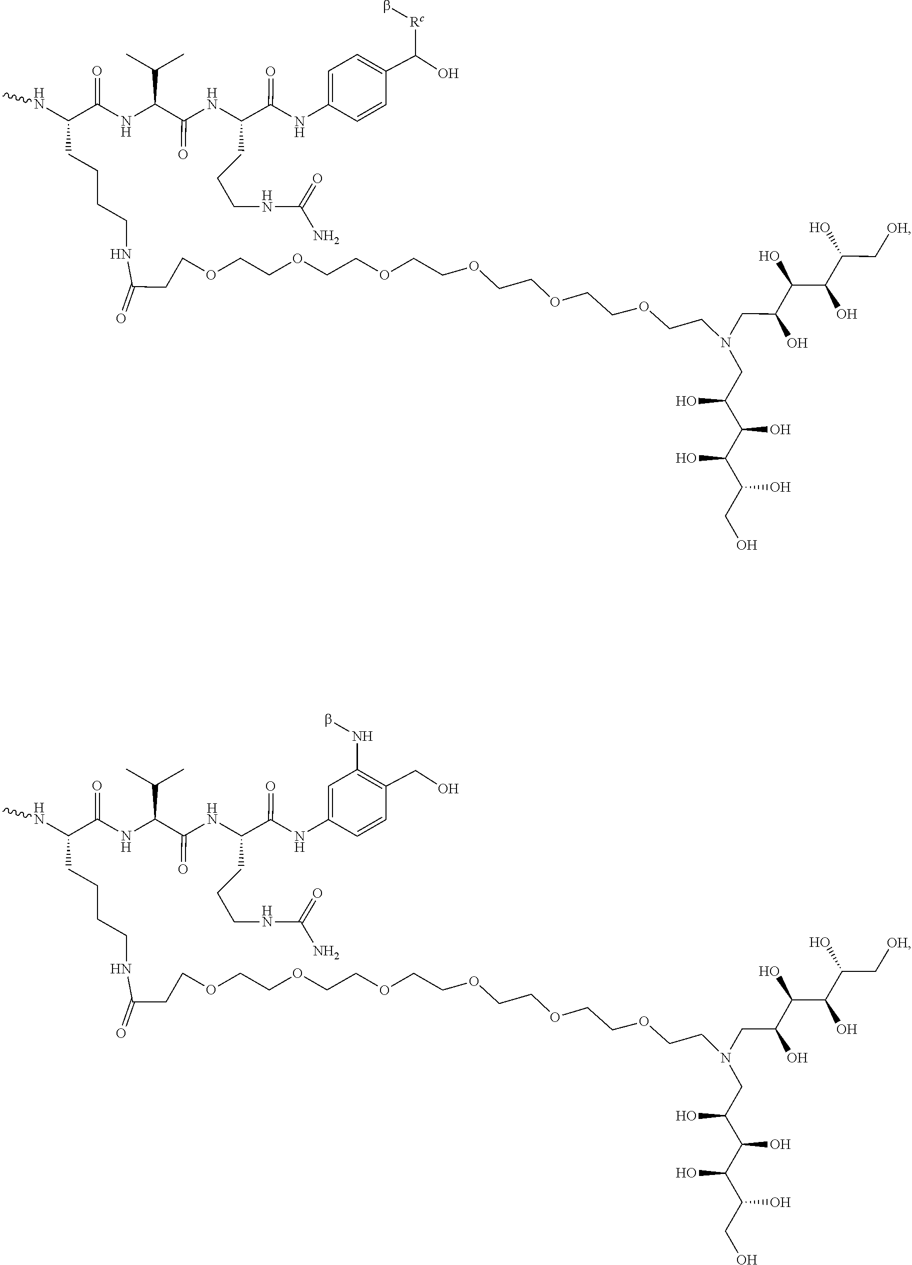 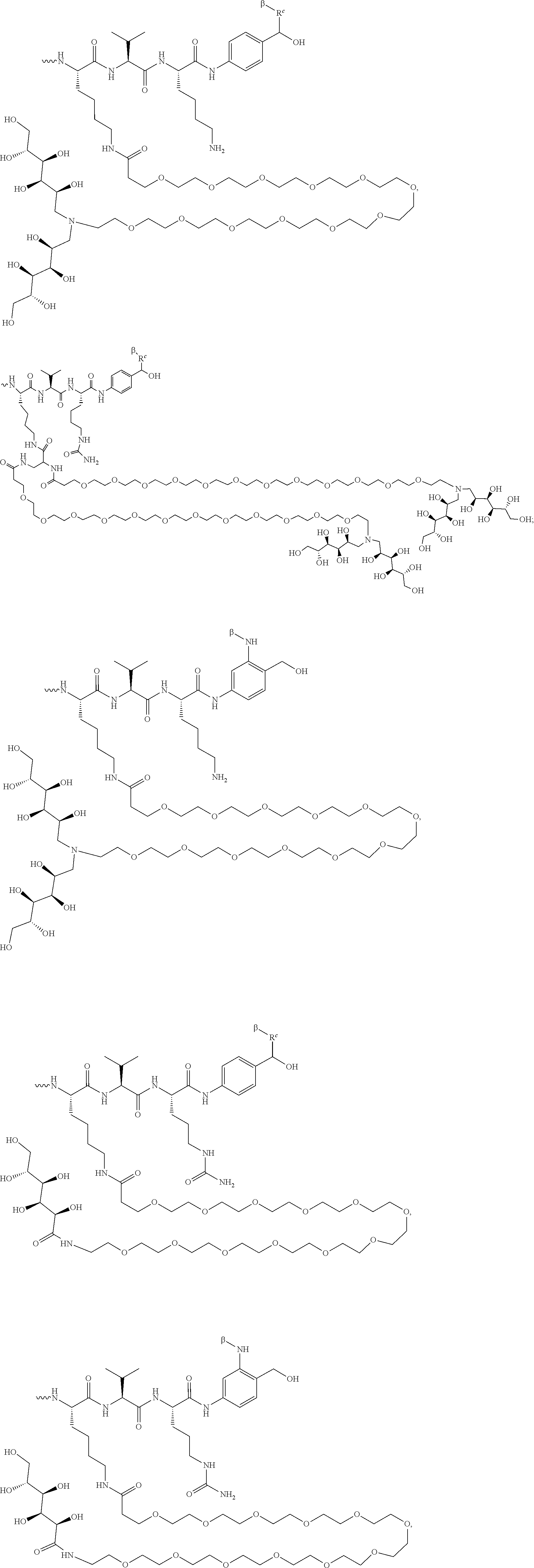 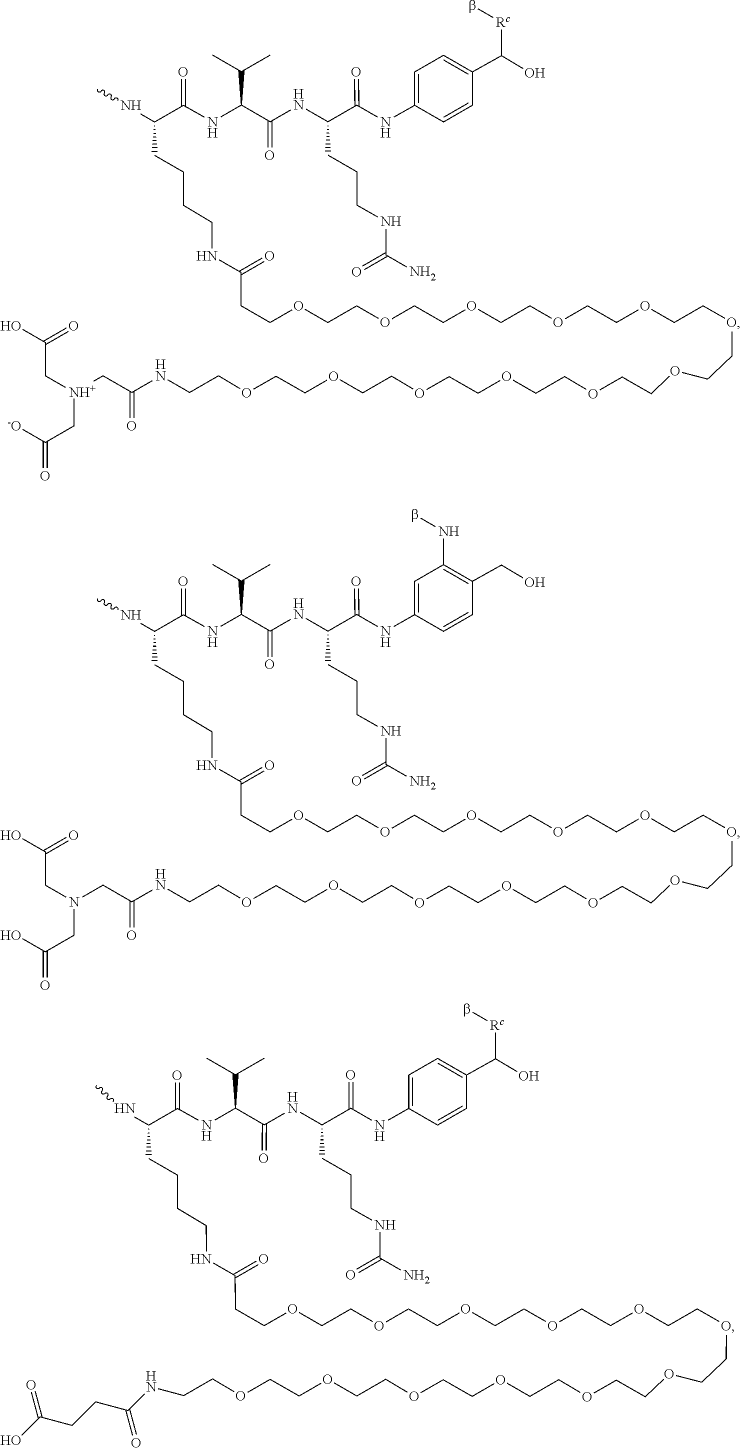 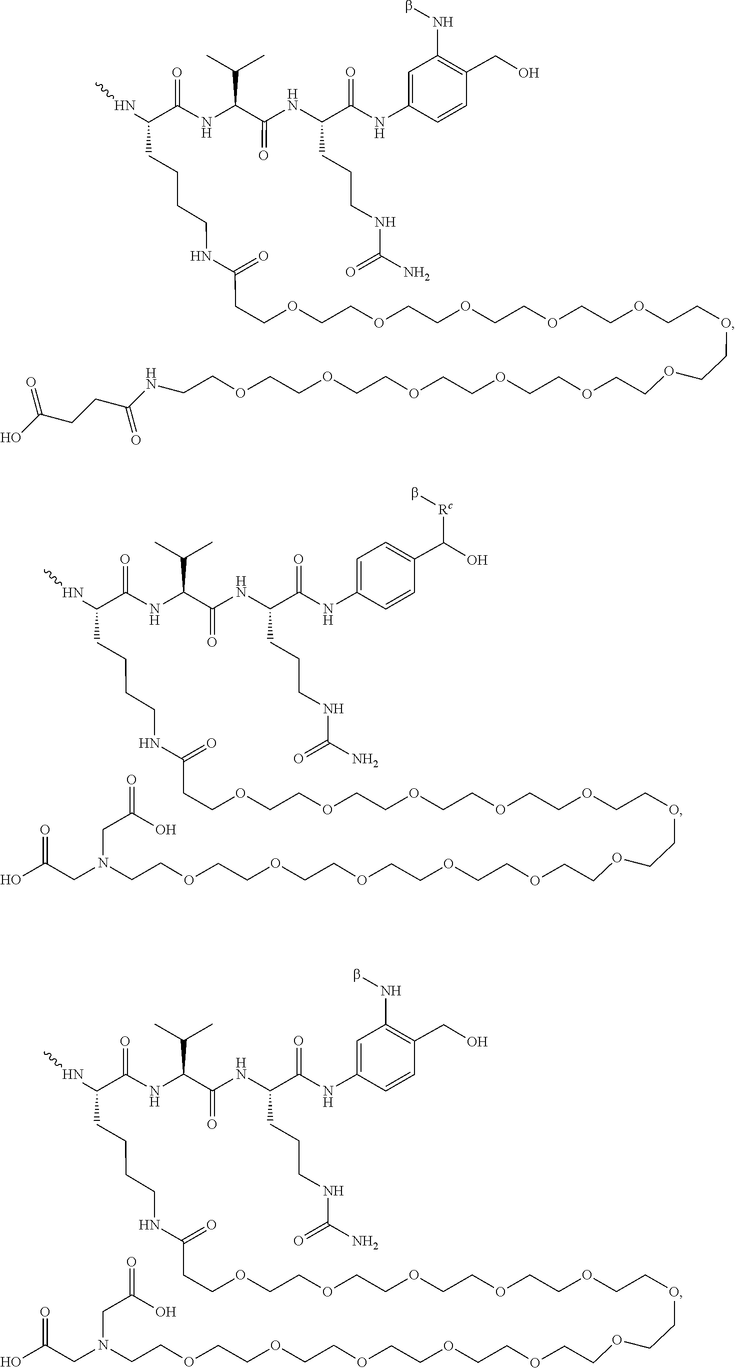 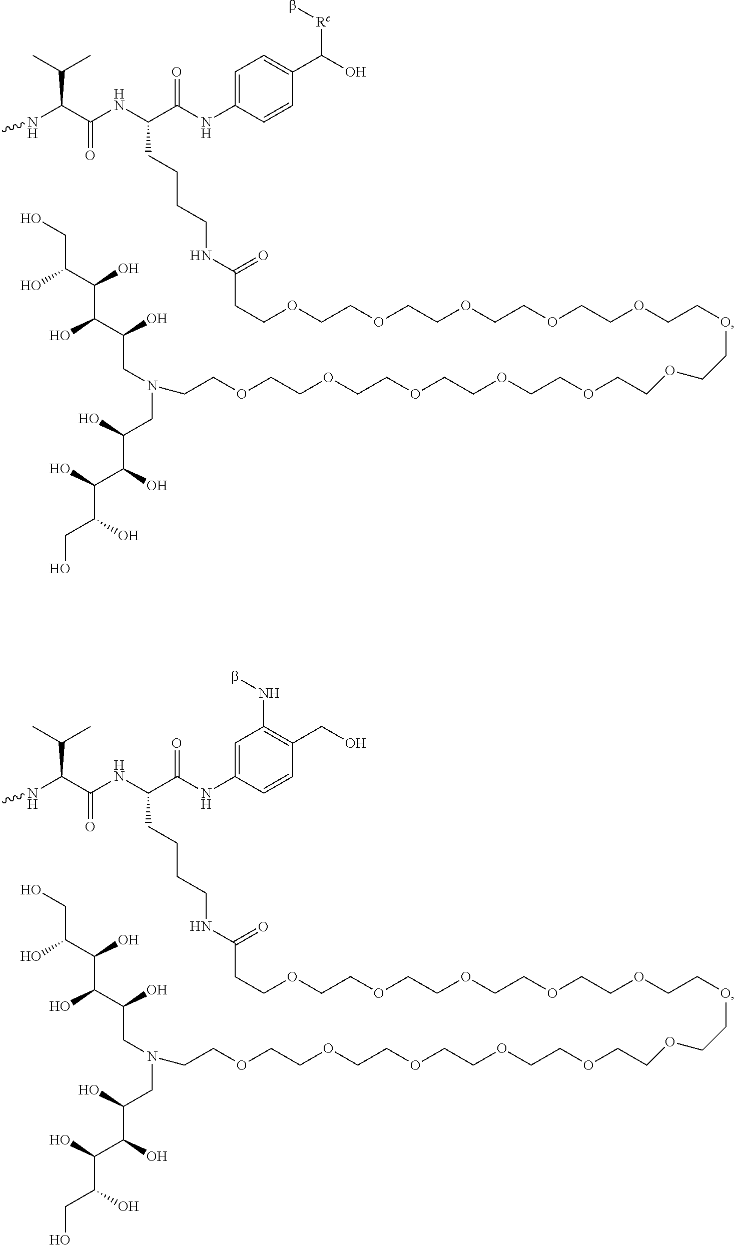 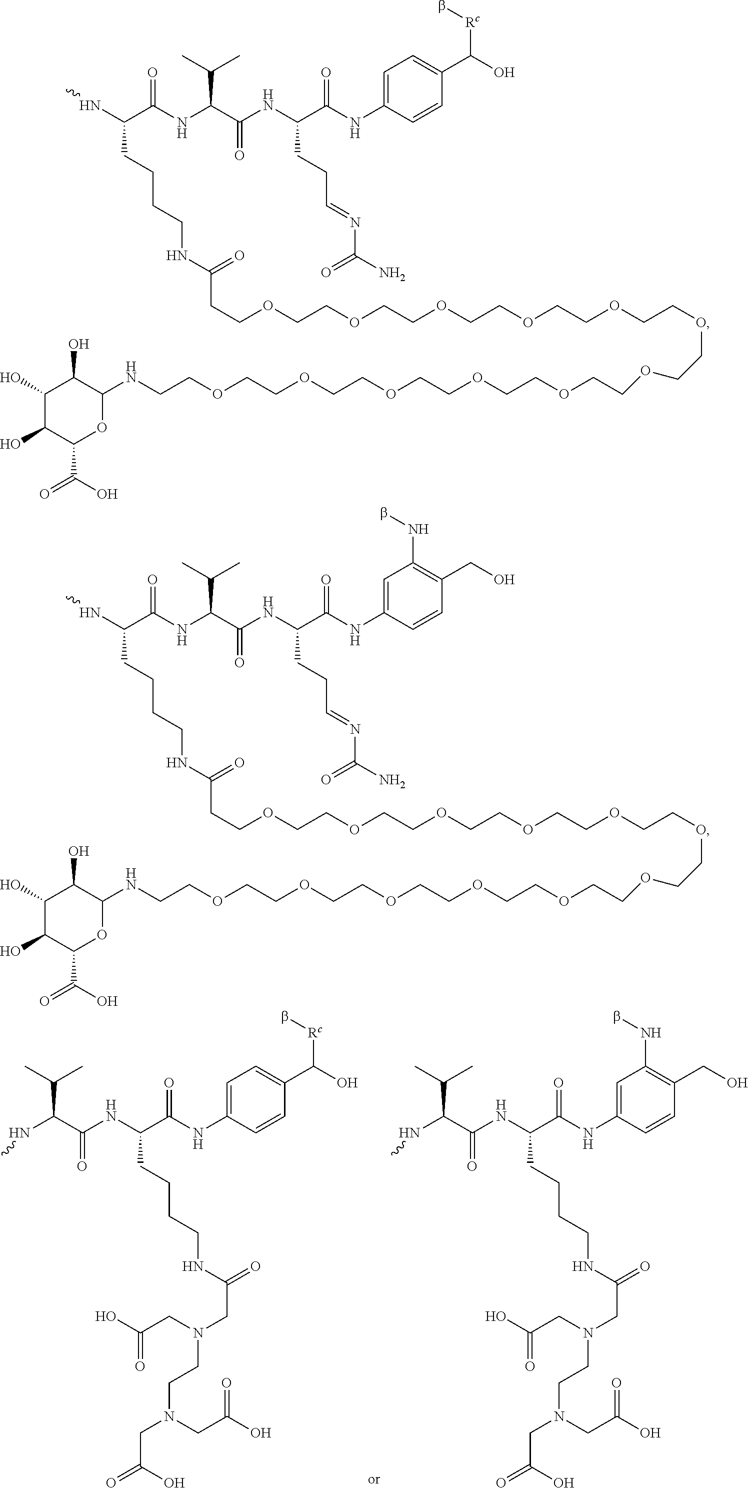
wherein
R^{c} is a bond or C₁₋₆ alkylene;
the wavy line on the amino group indicates an attachment site for the stretcher group; or, prior to attachment to the stretcher group, indicates H;
β— is the attachment site to the at least one polar group; and
the H on the benzylic OH is optionally replaced with a bond to one of the drug units or to the attachment site to at least one of the drug units.

68. The conjugate of claim 30, having one of the following structures: 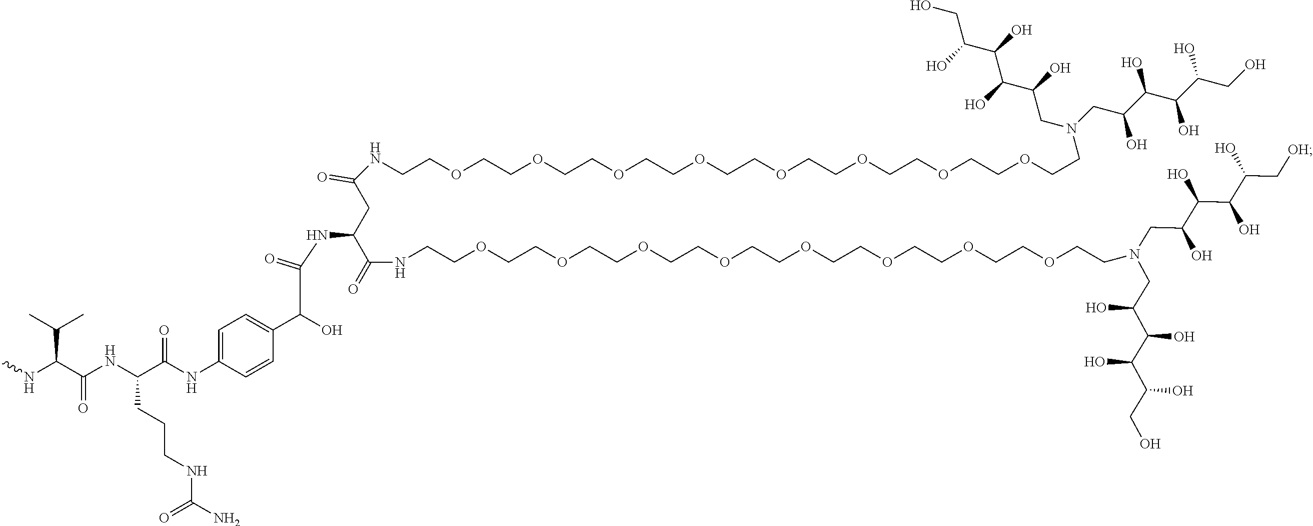 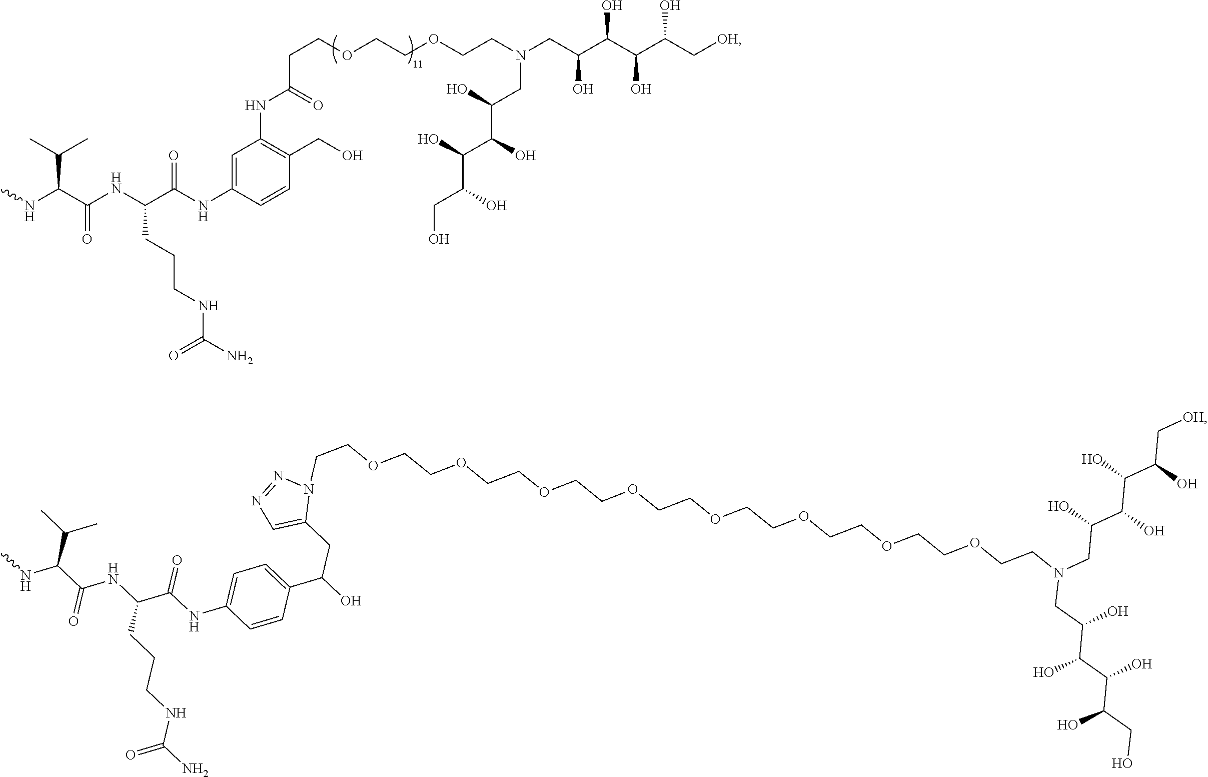 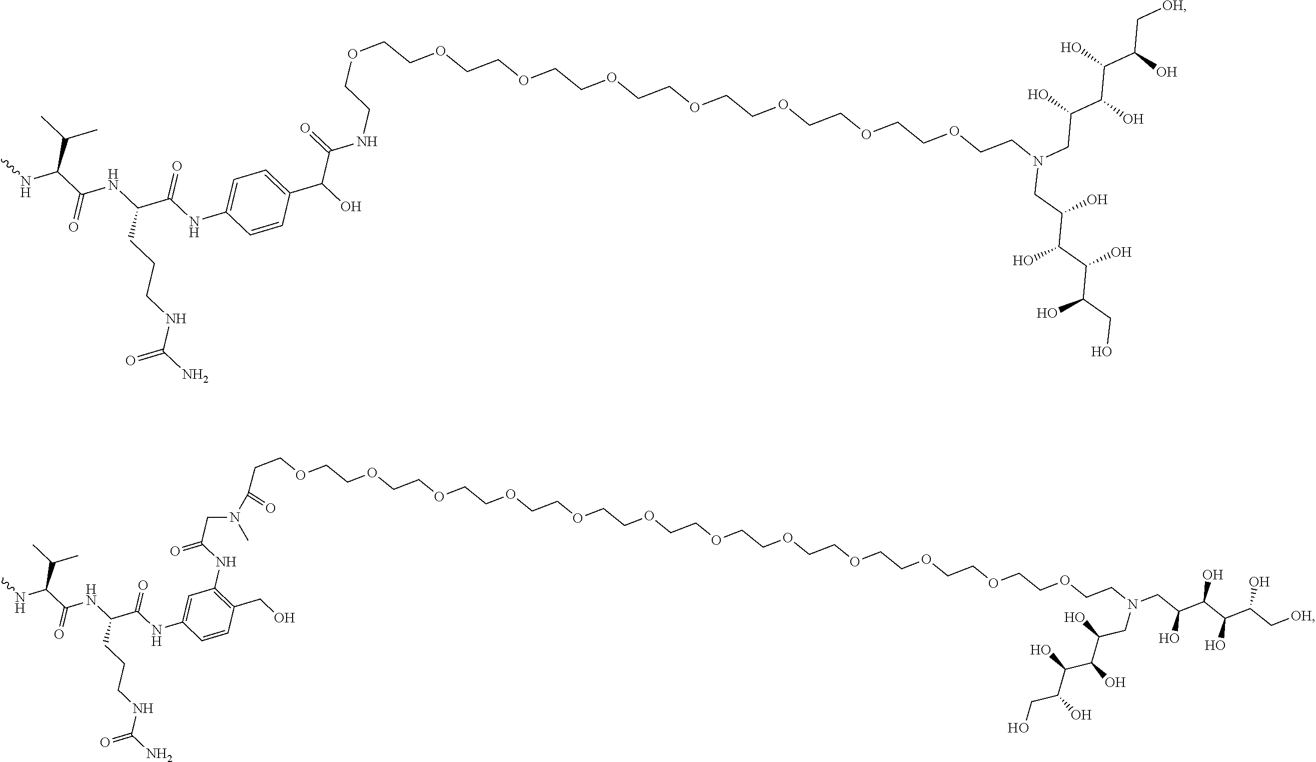 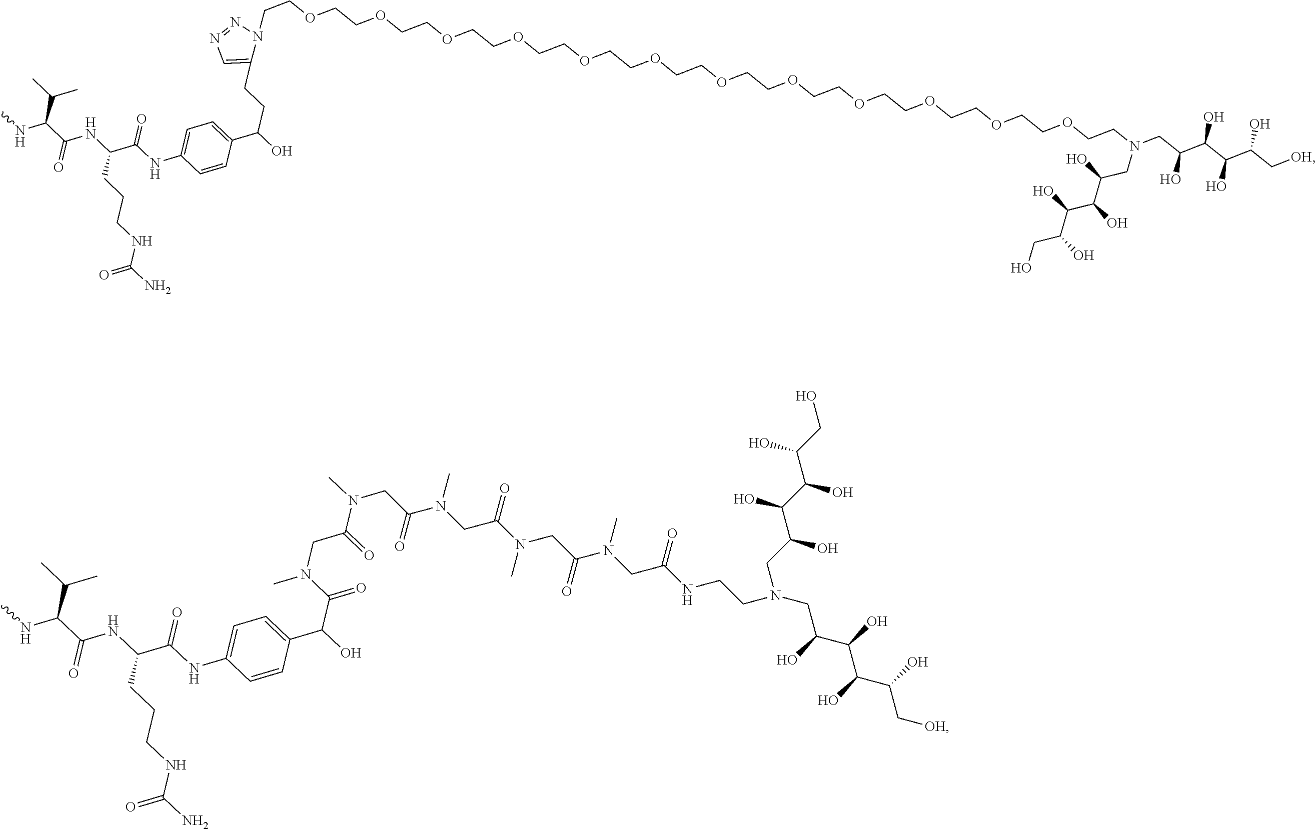 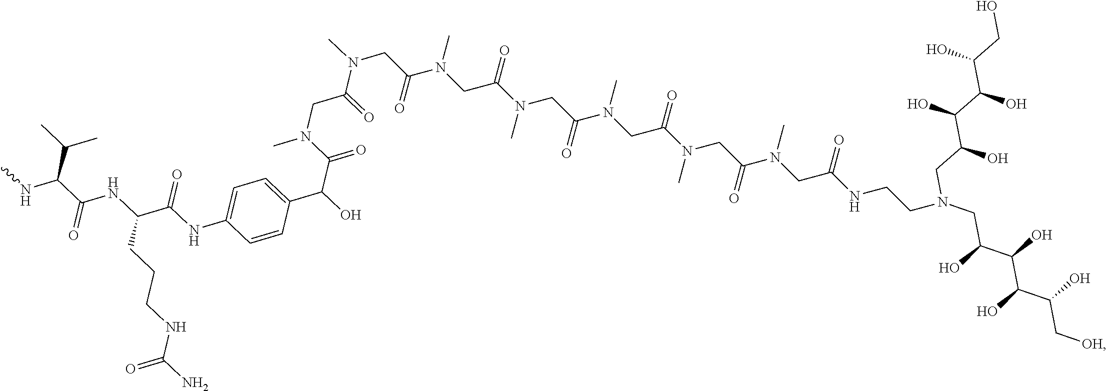 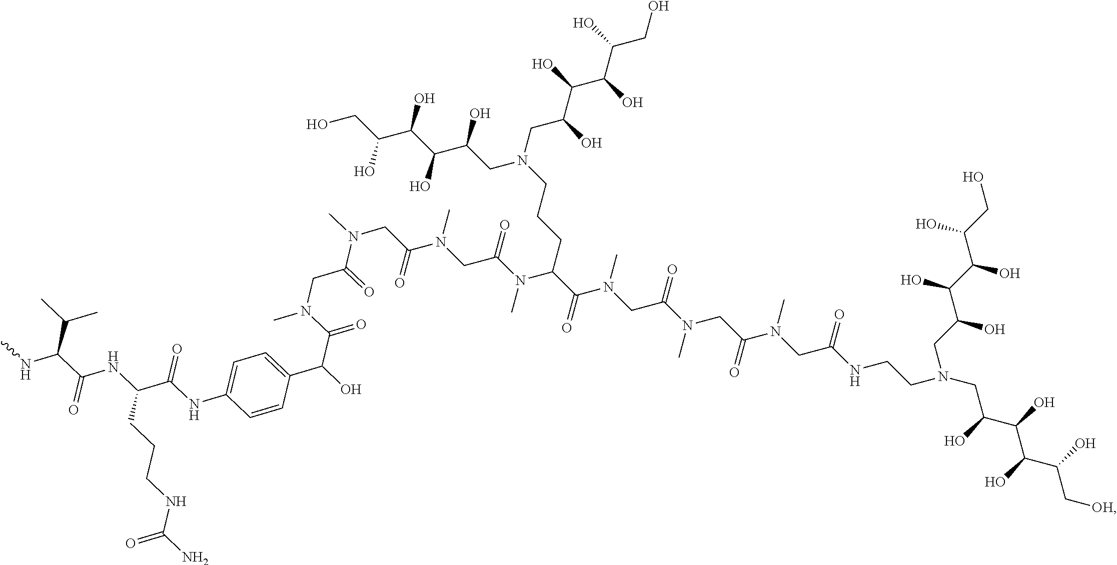 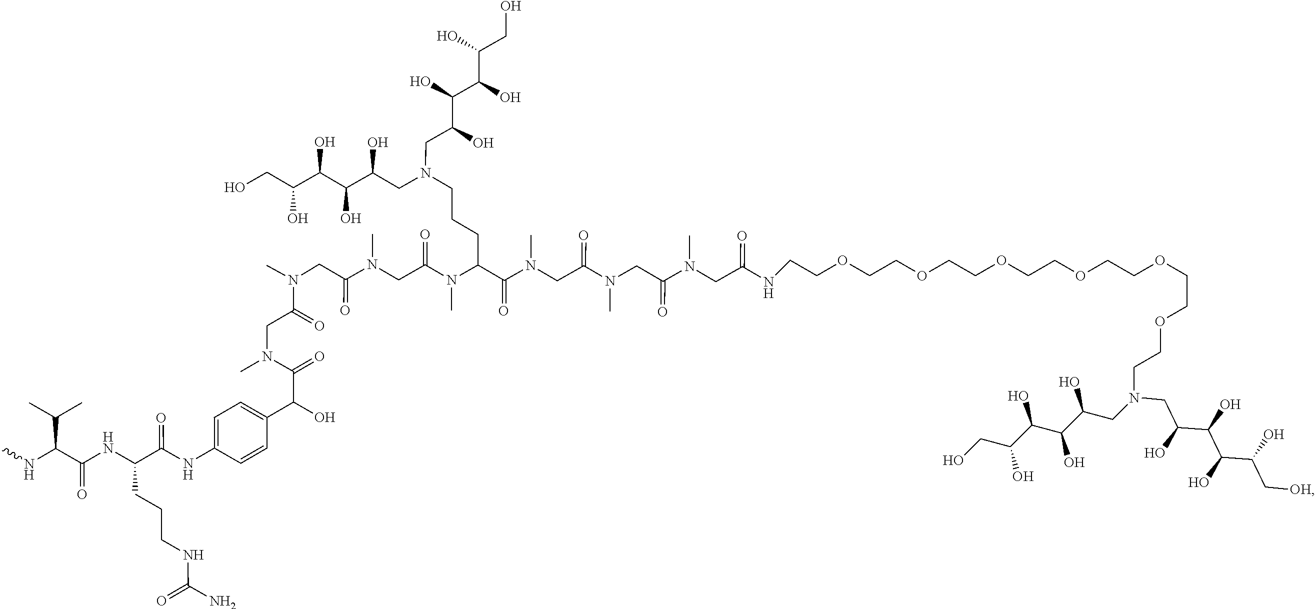 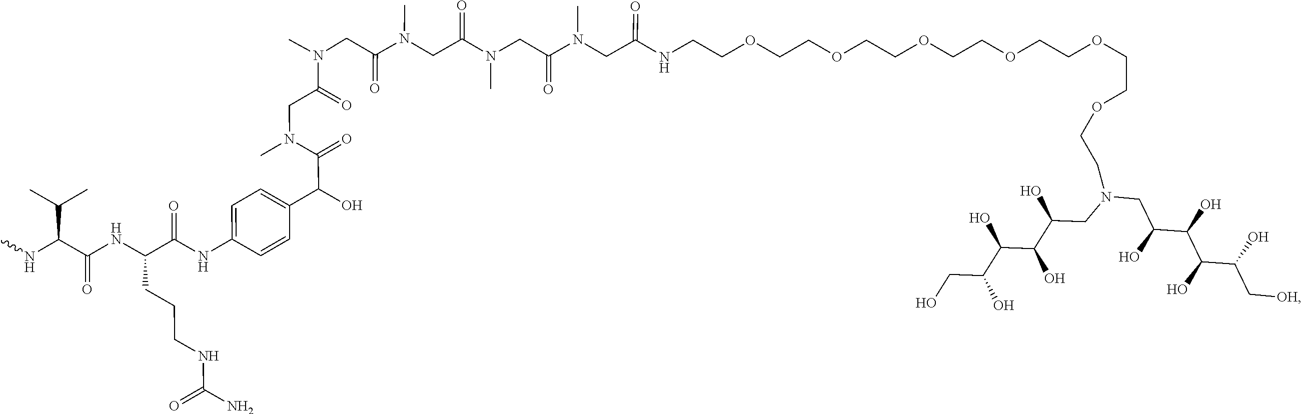 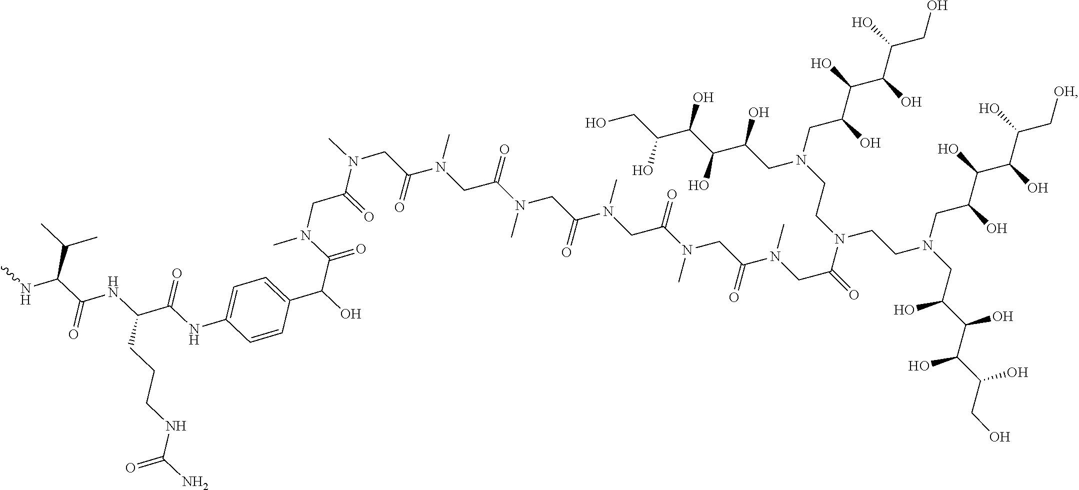 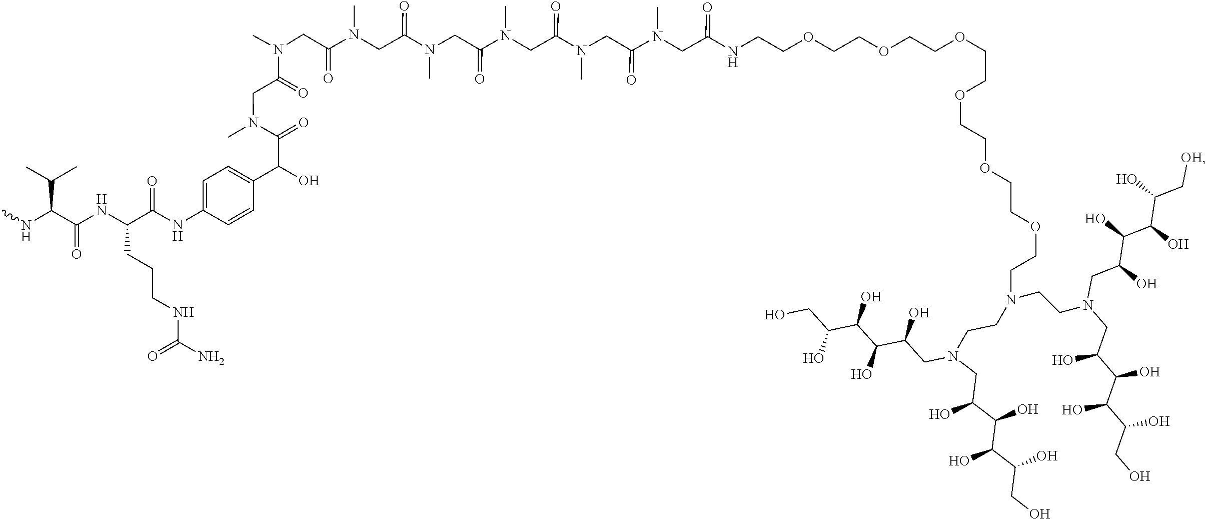 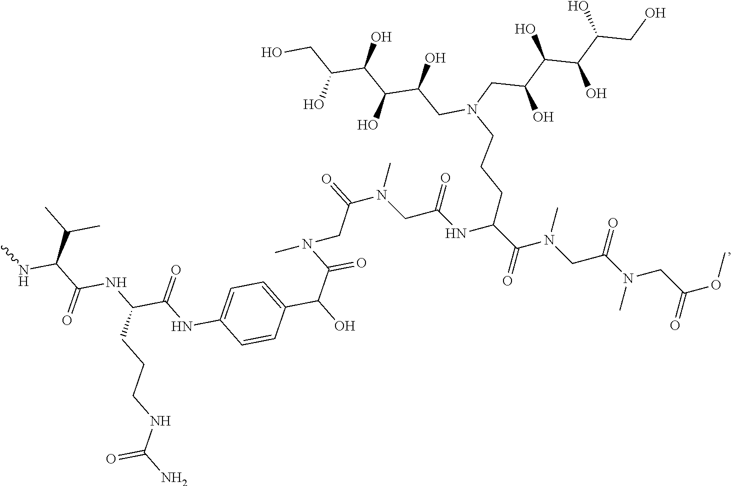 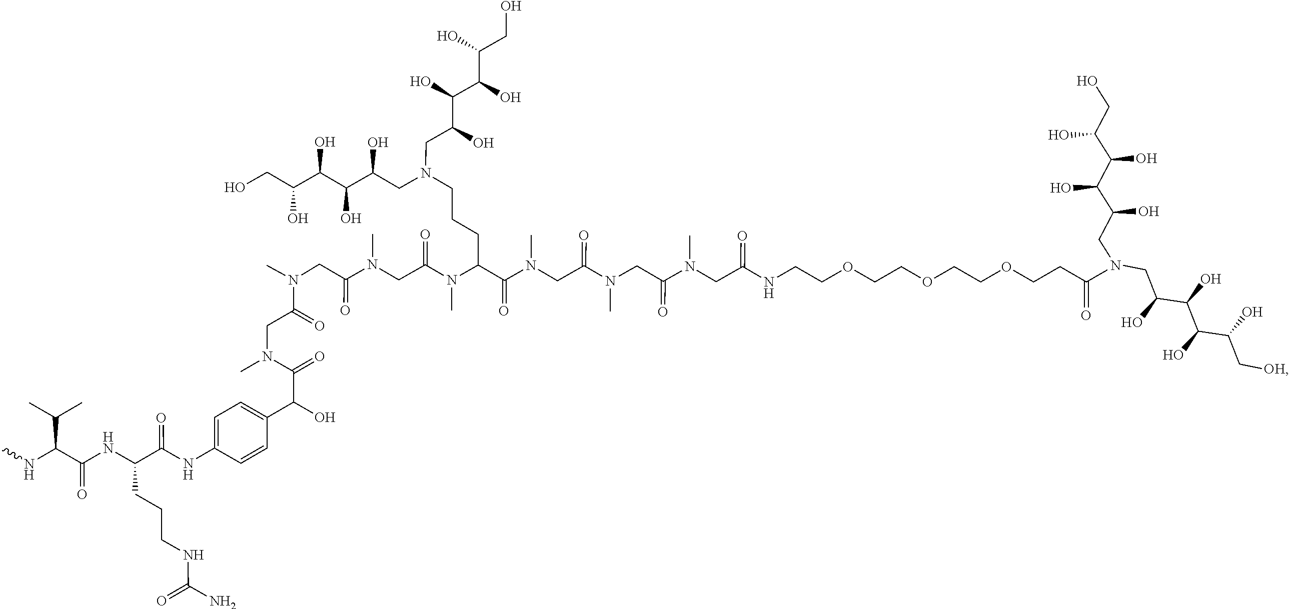 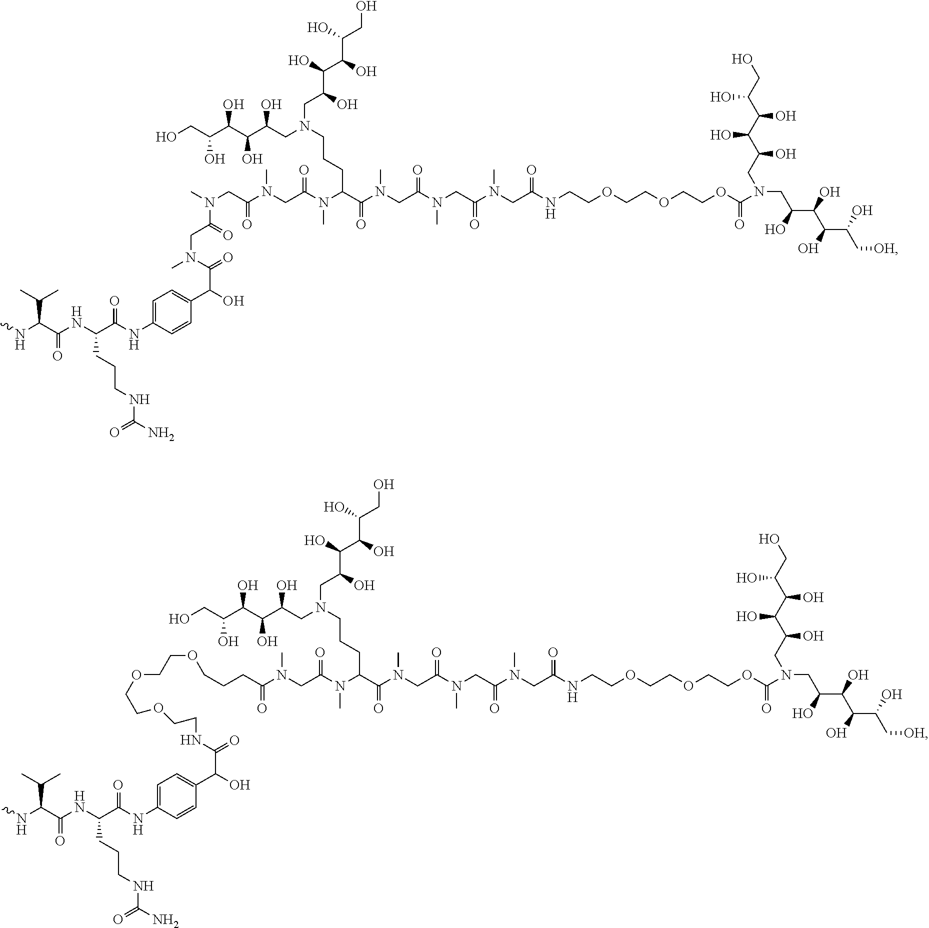 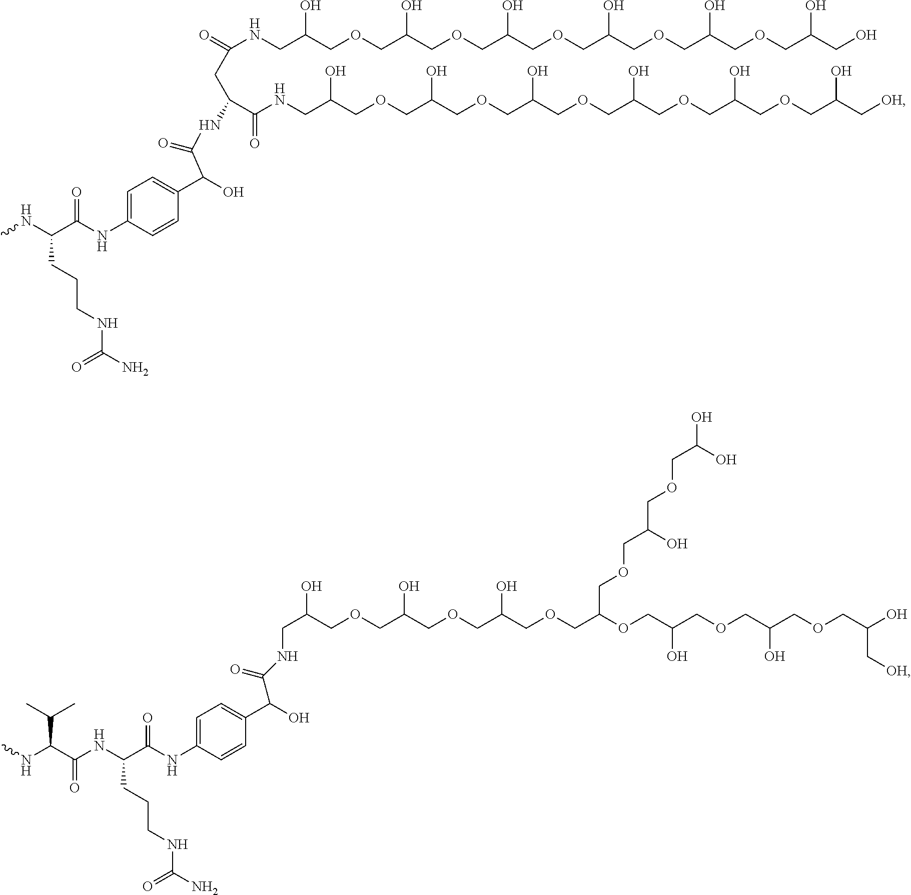 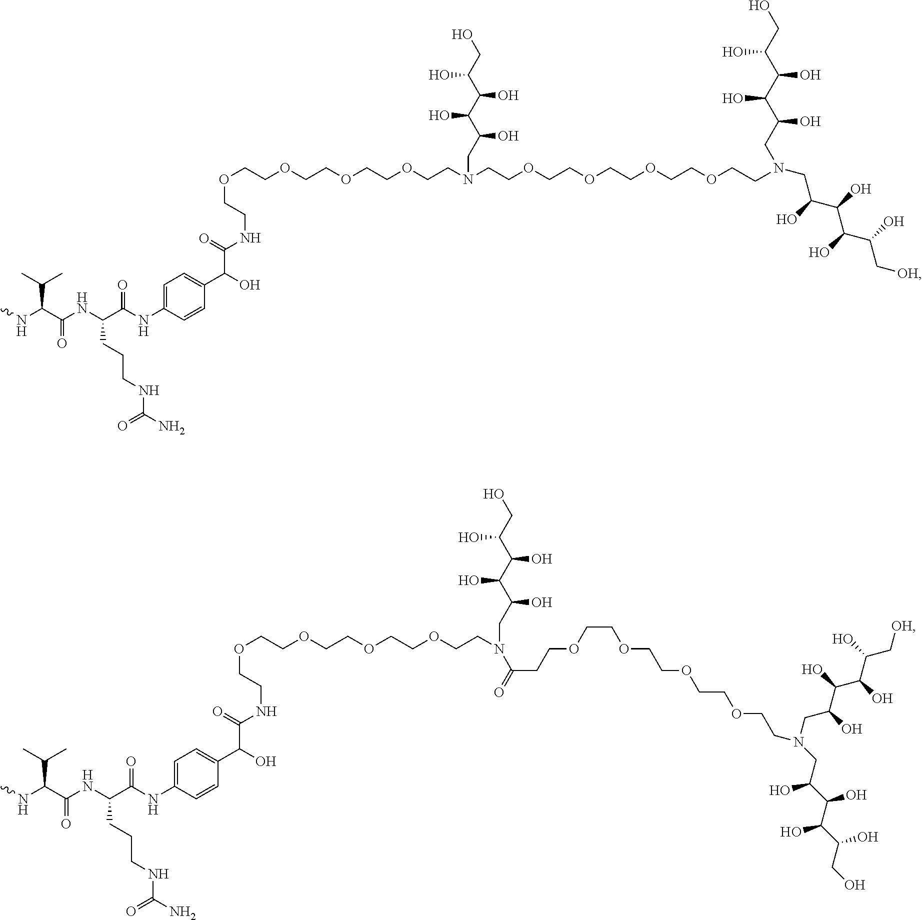 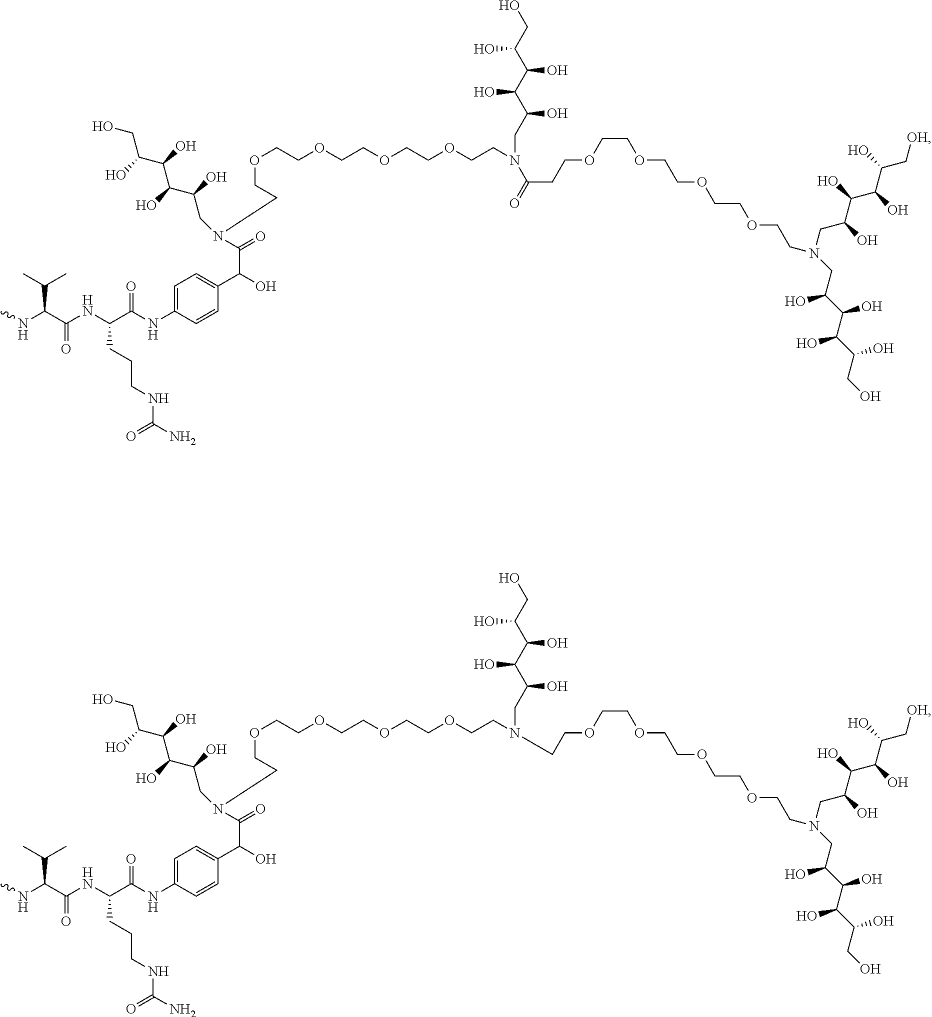 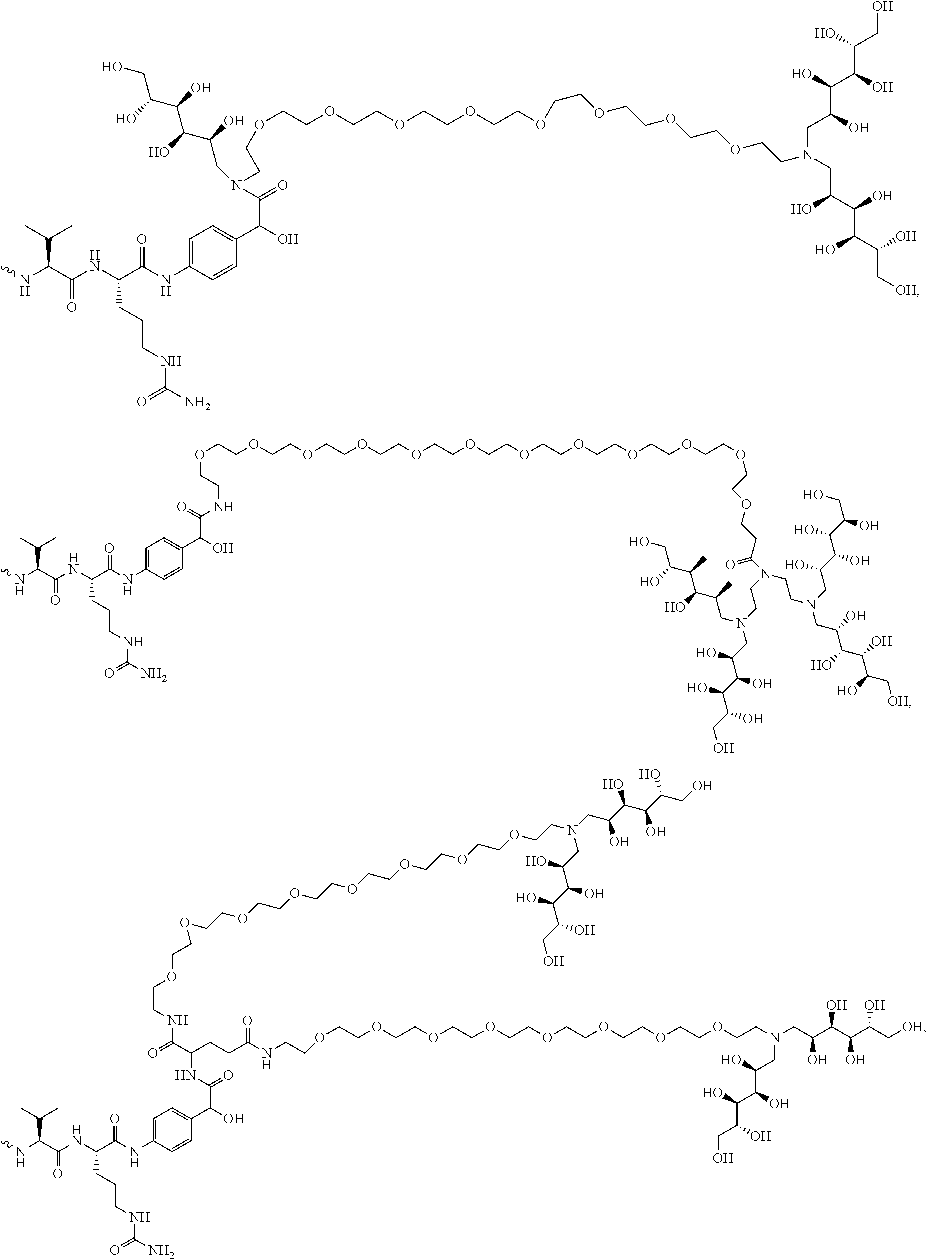 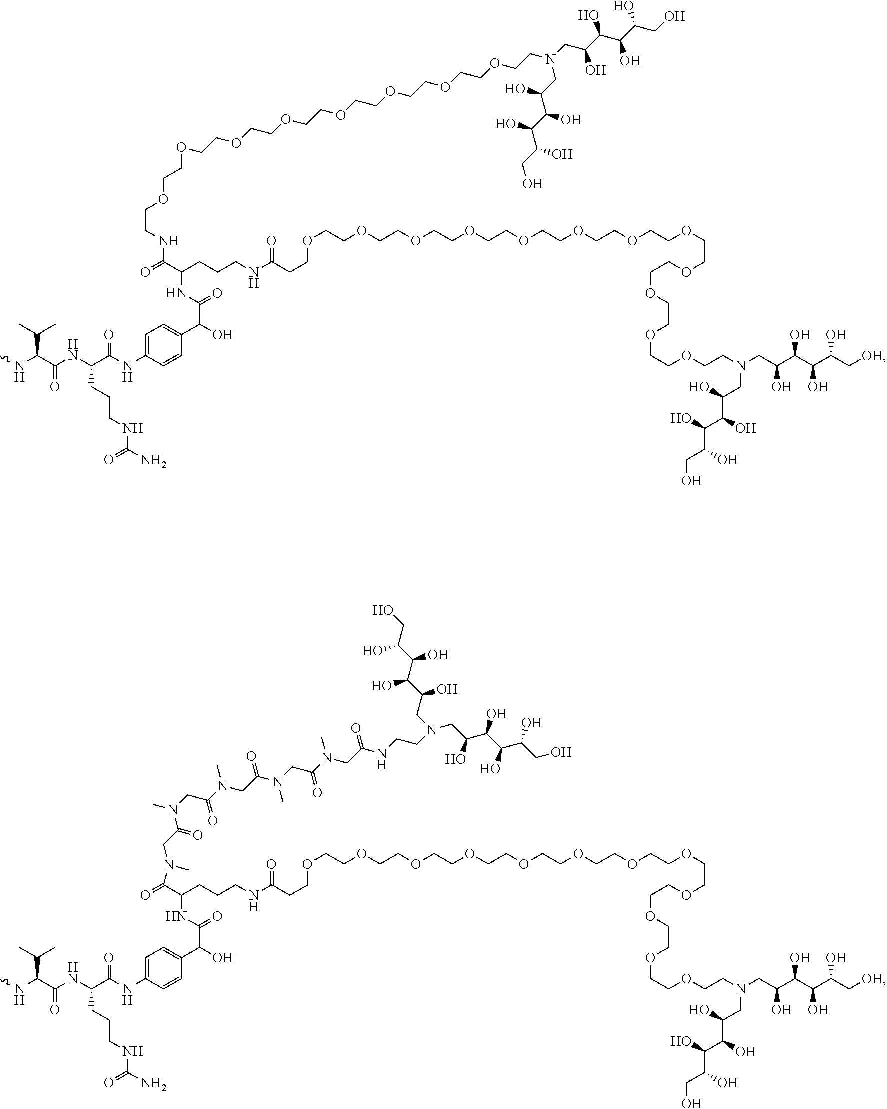 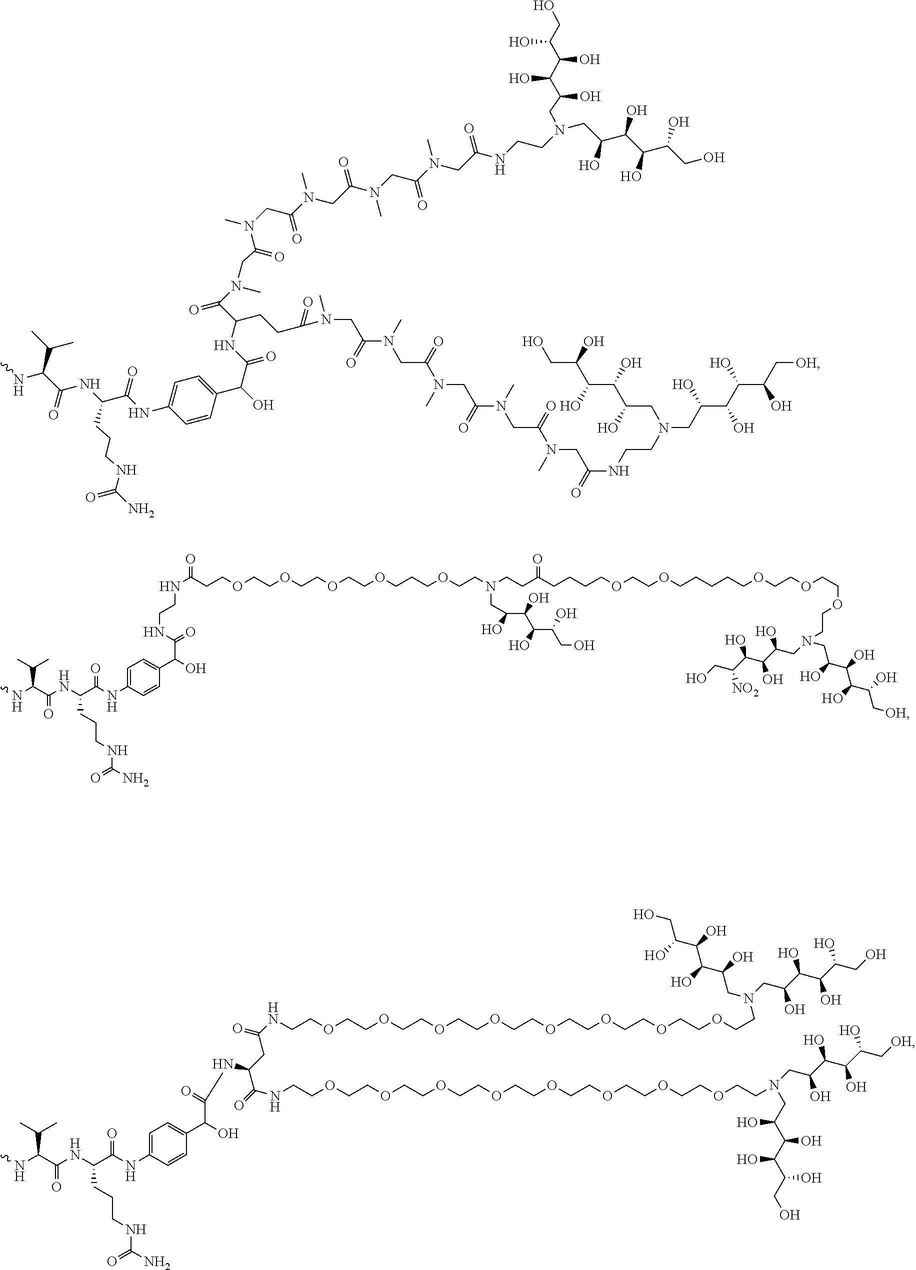 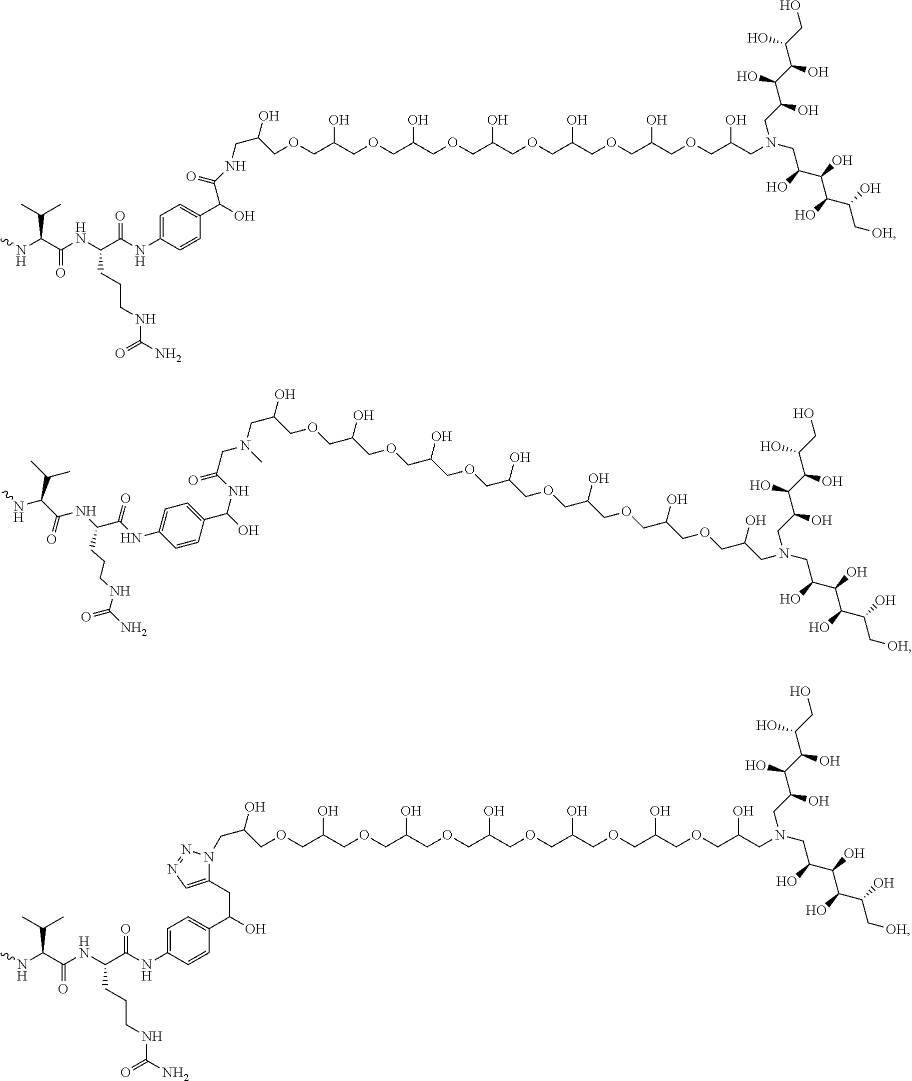 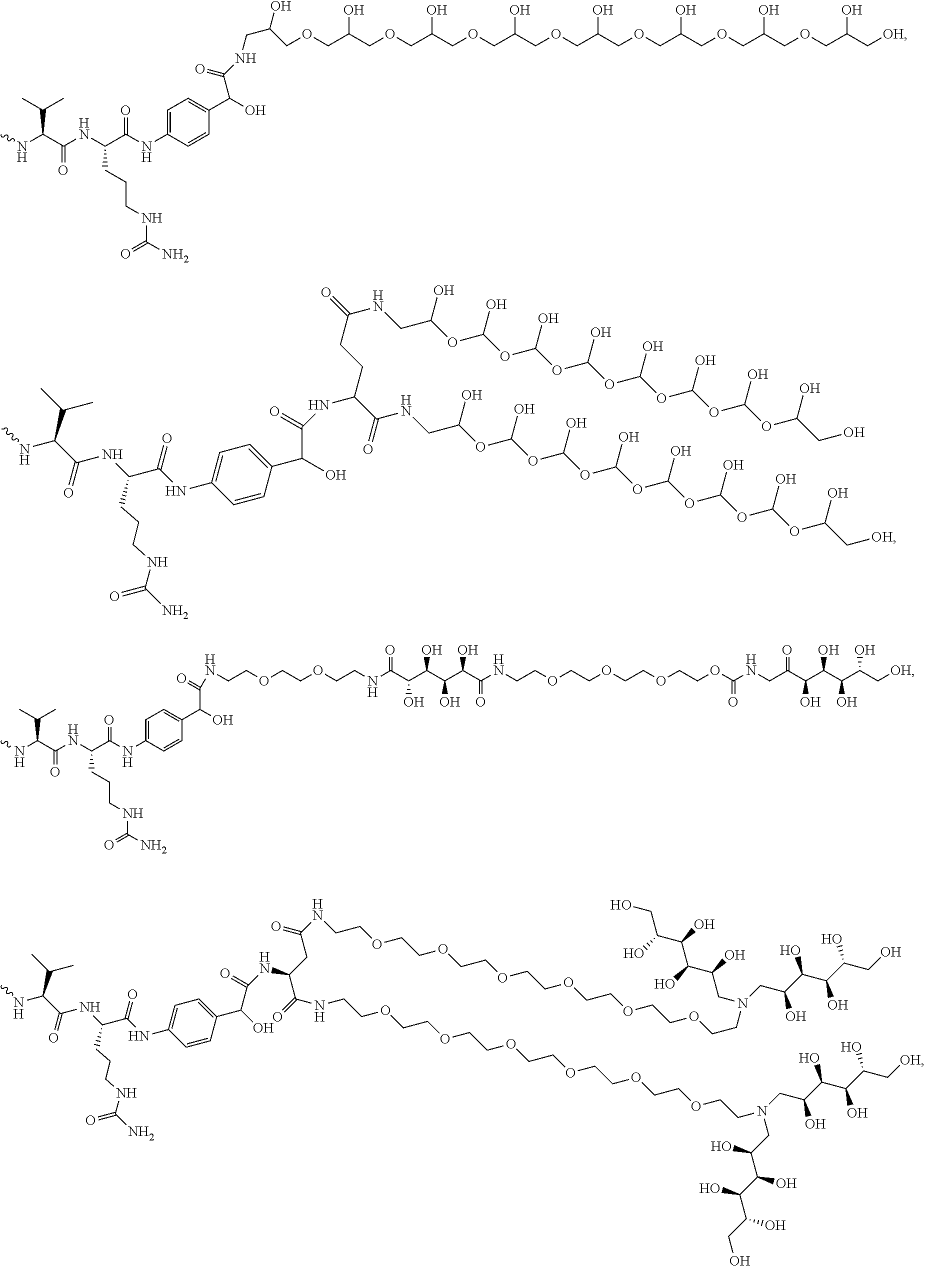 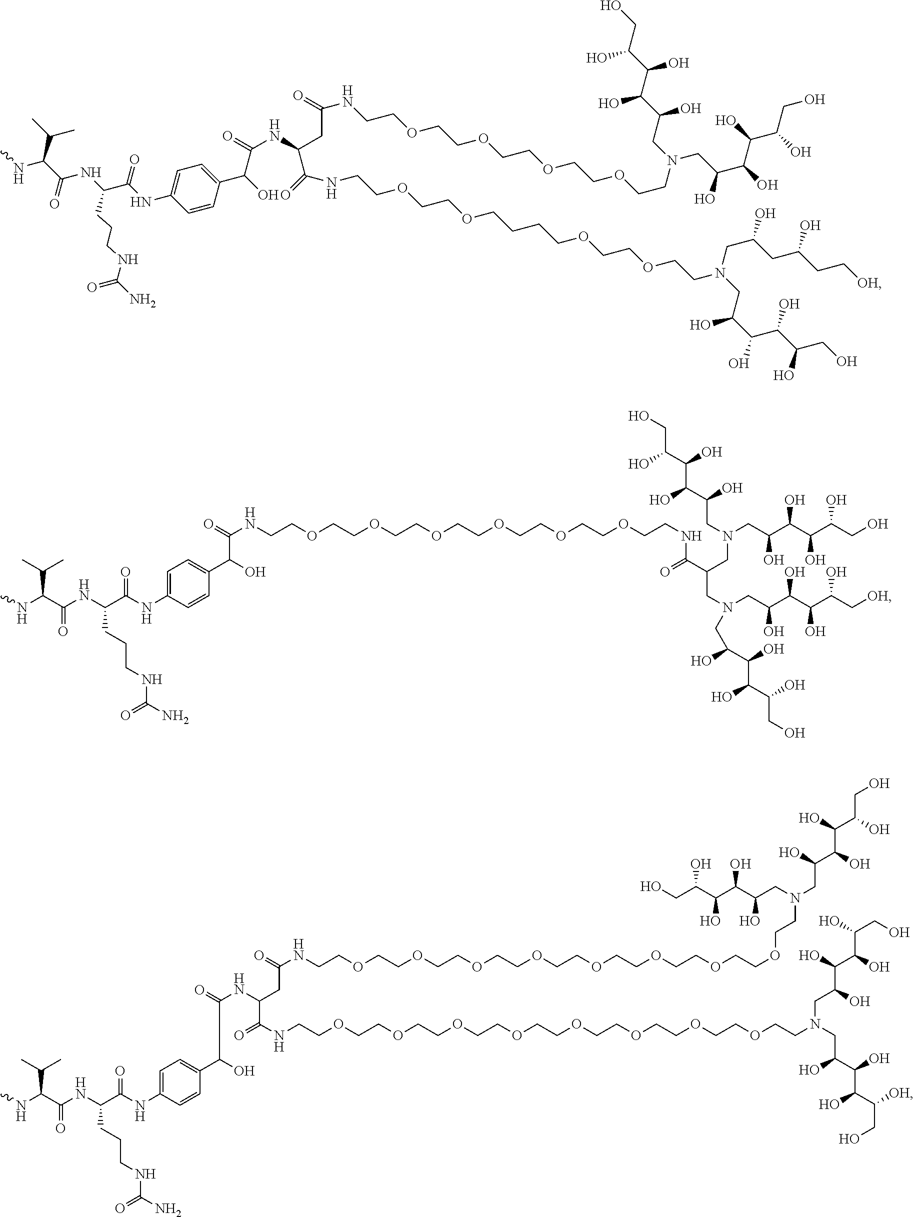 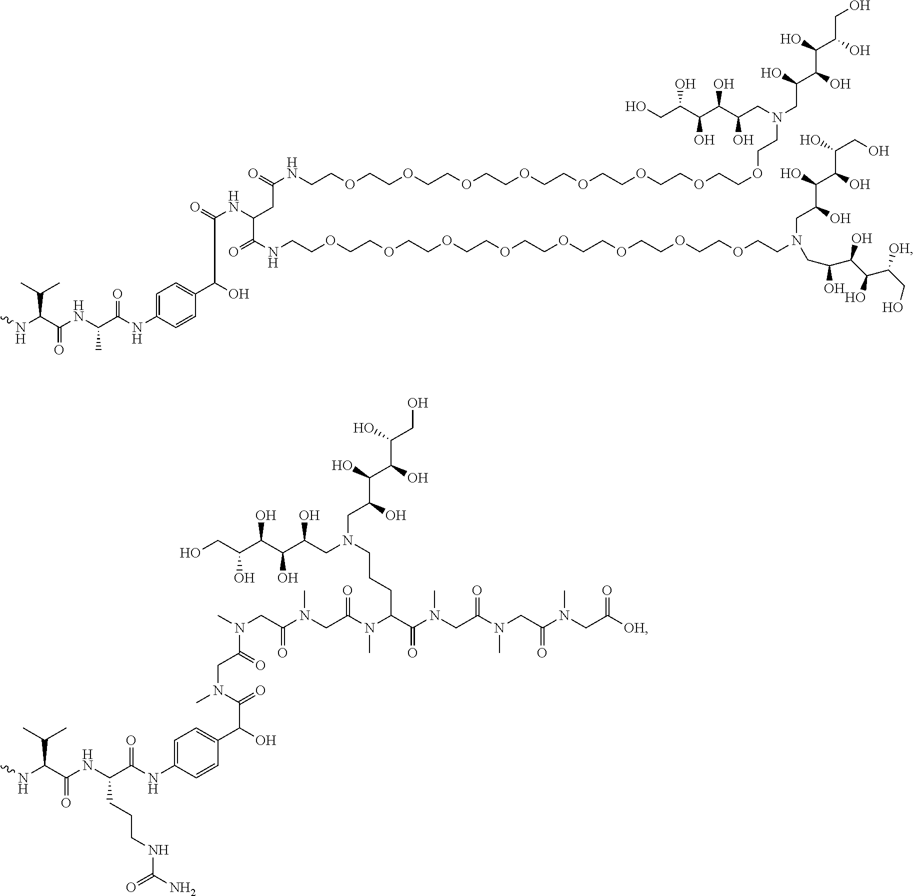 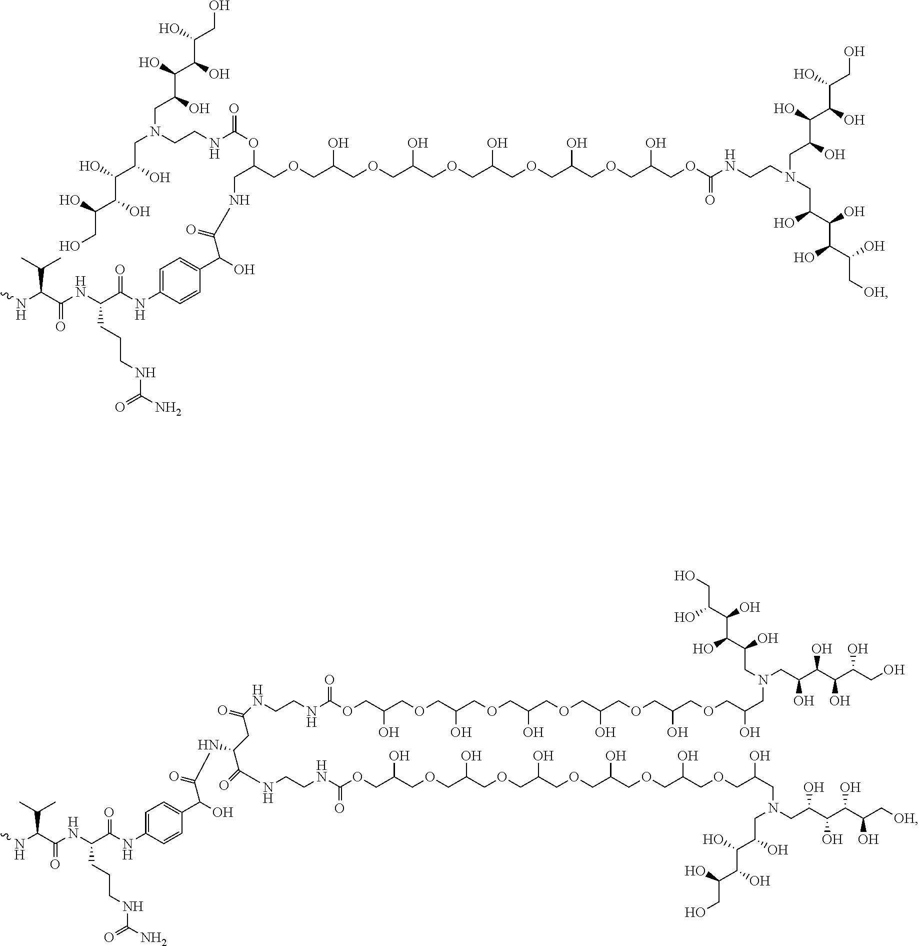 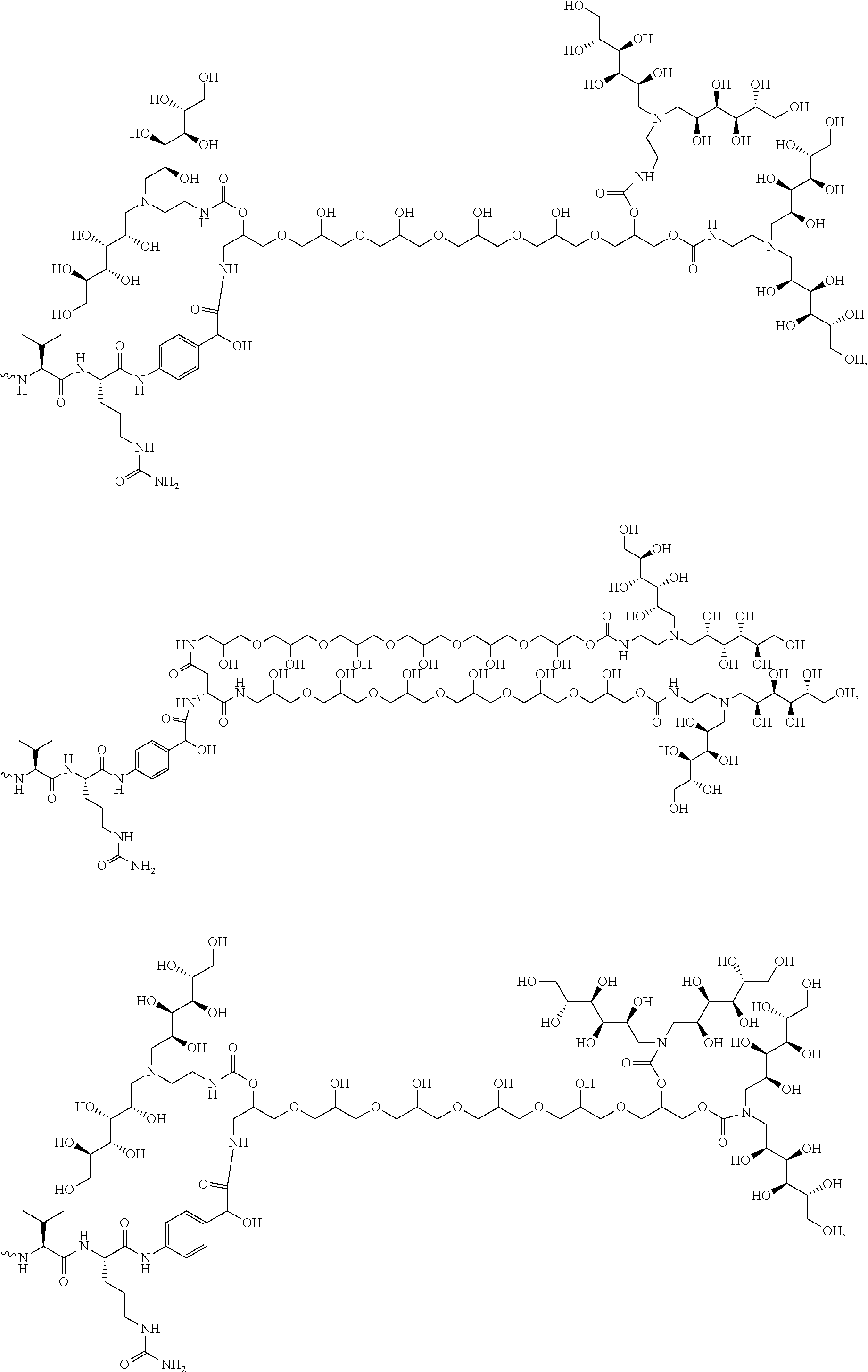 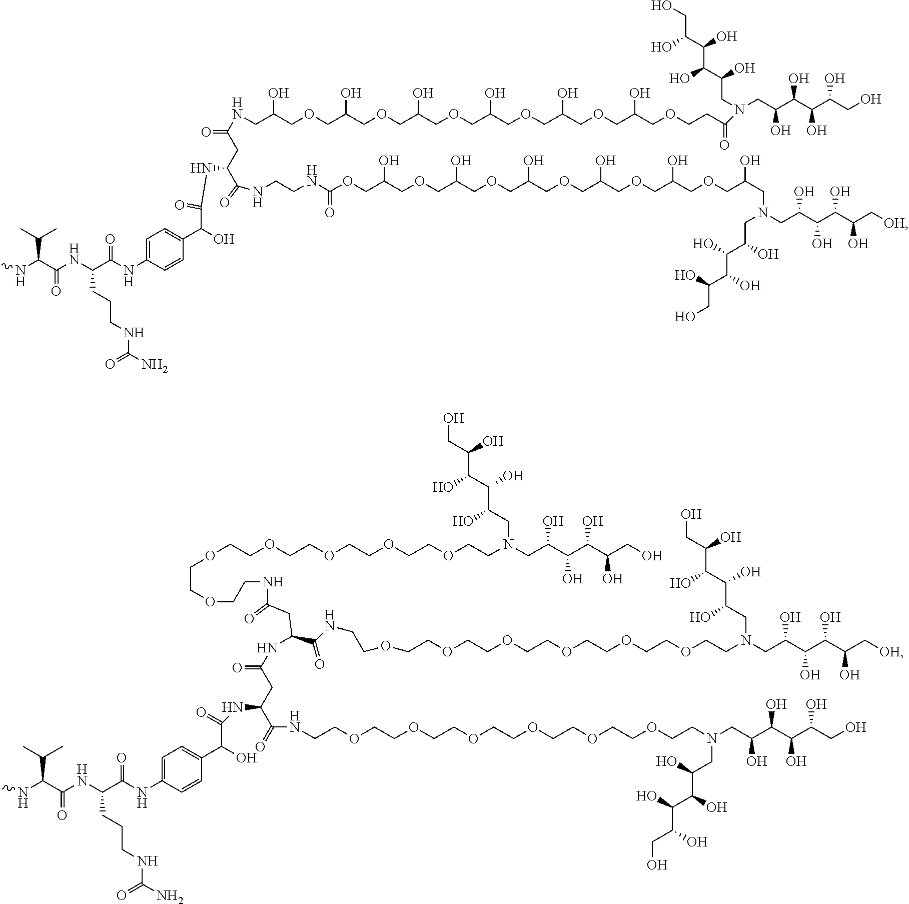 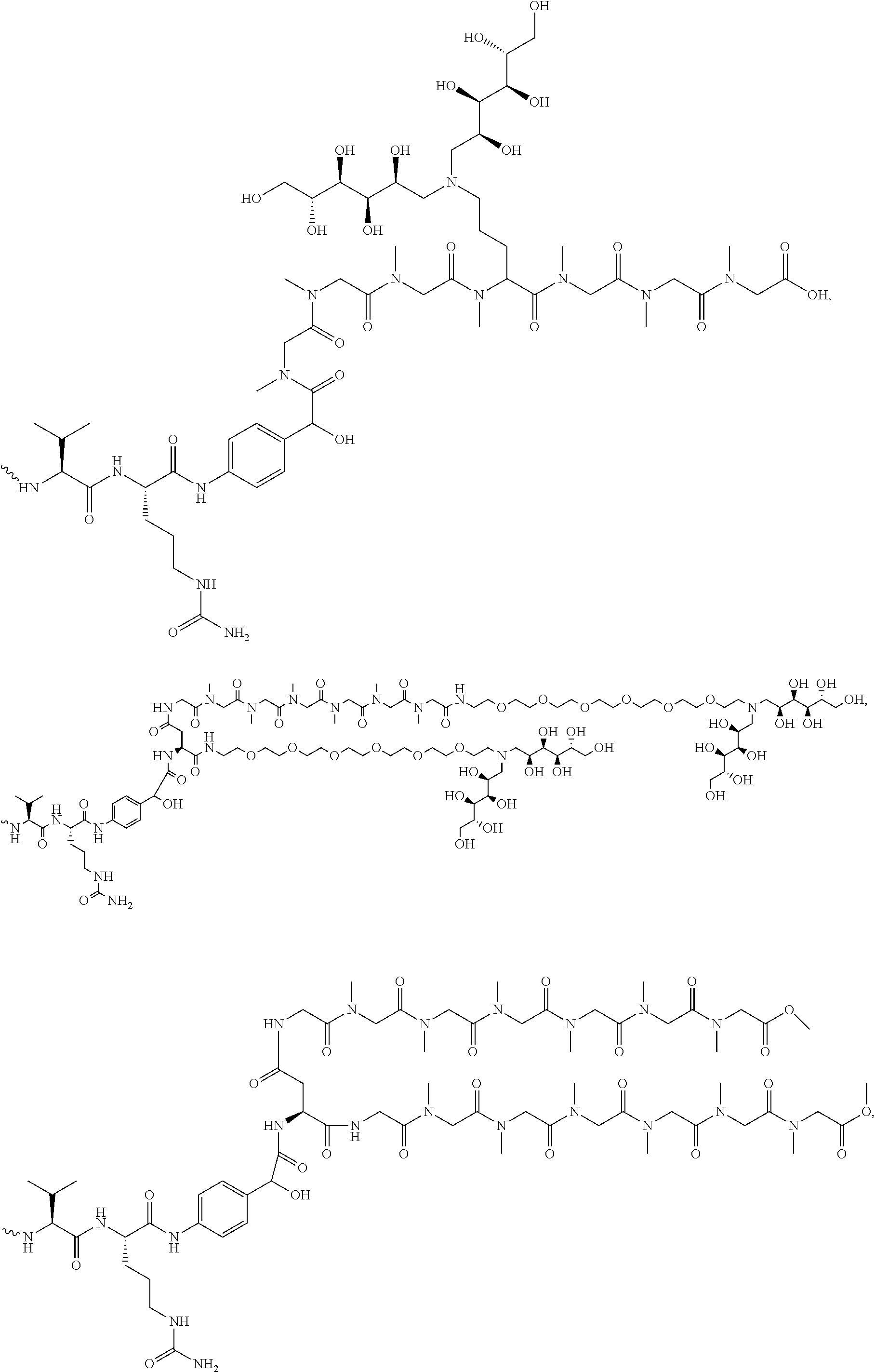 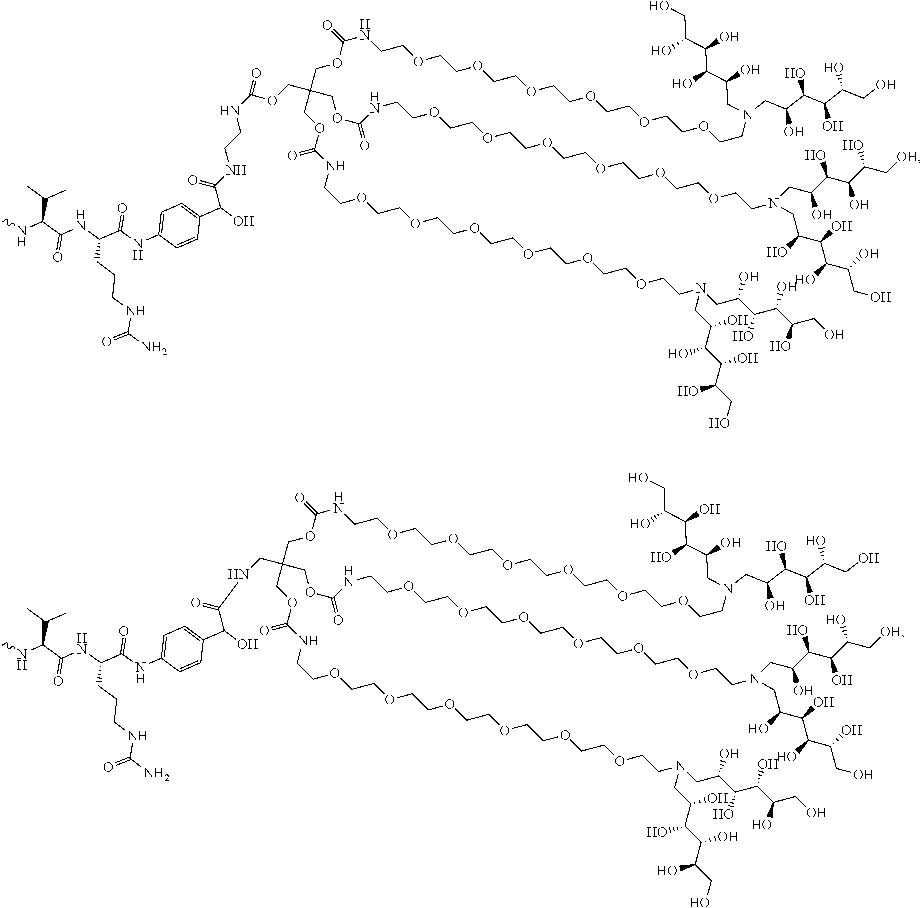 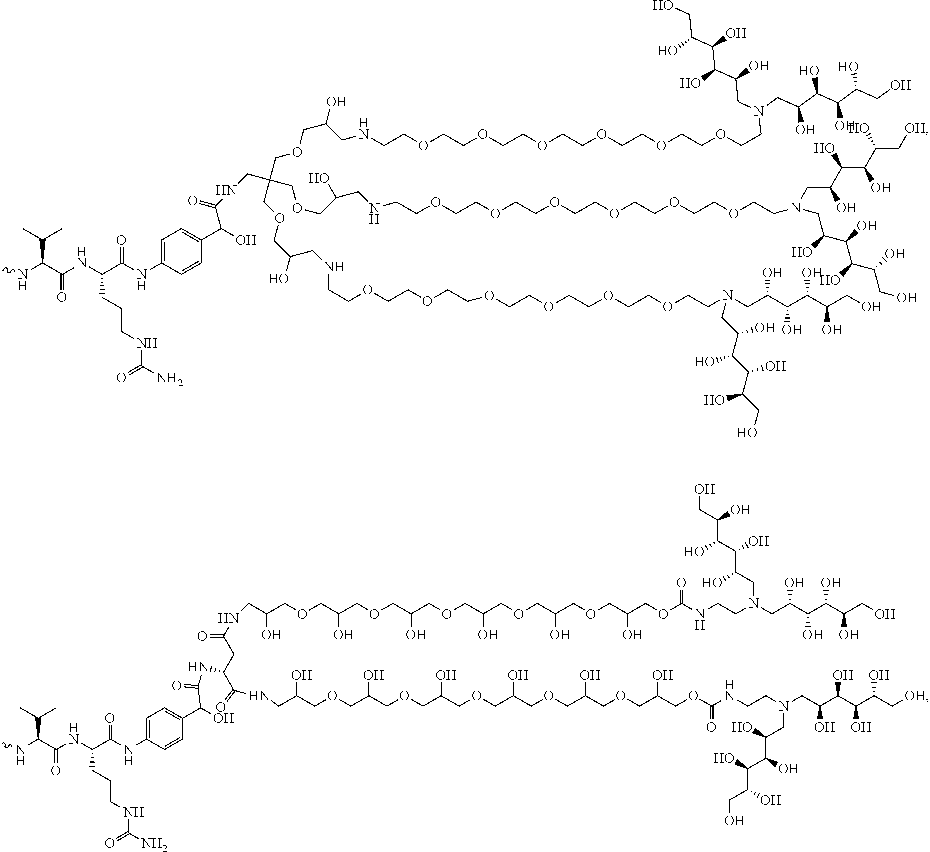 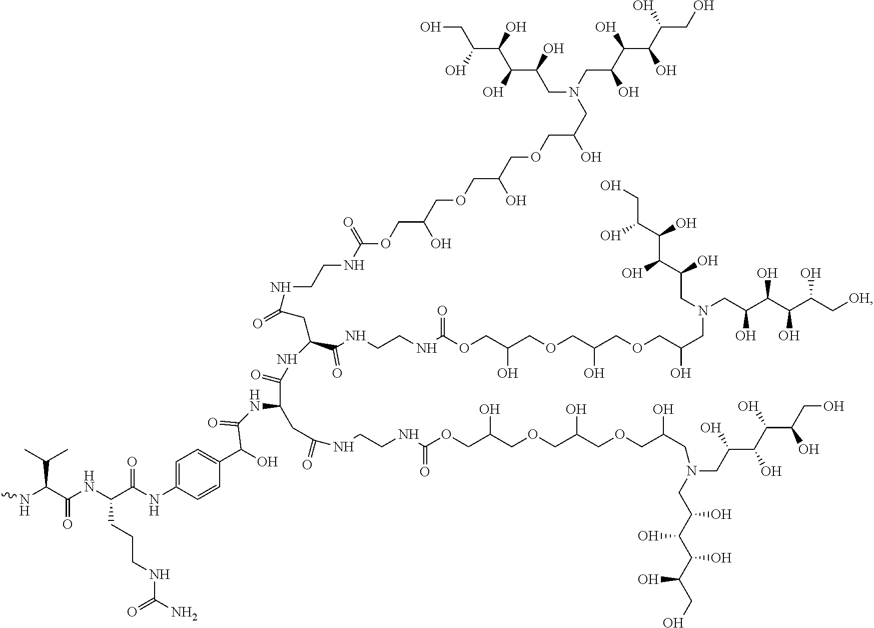 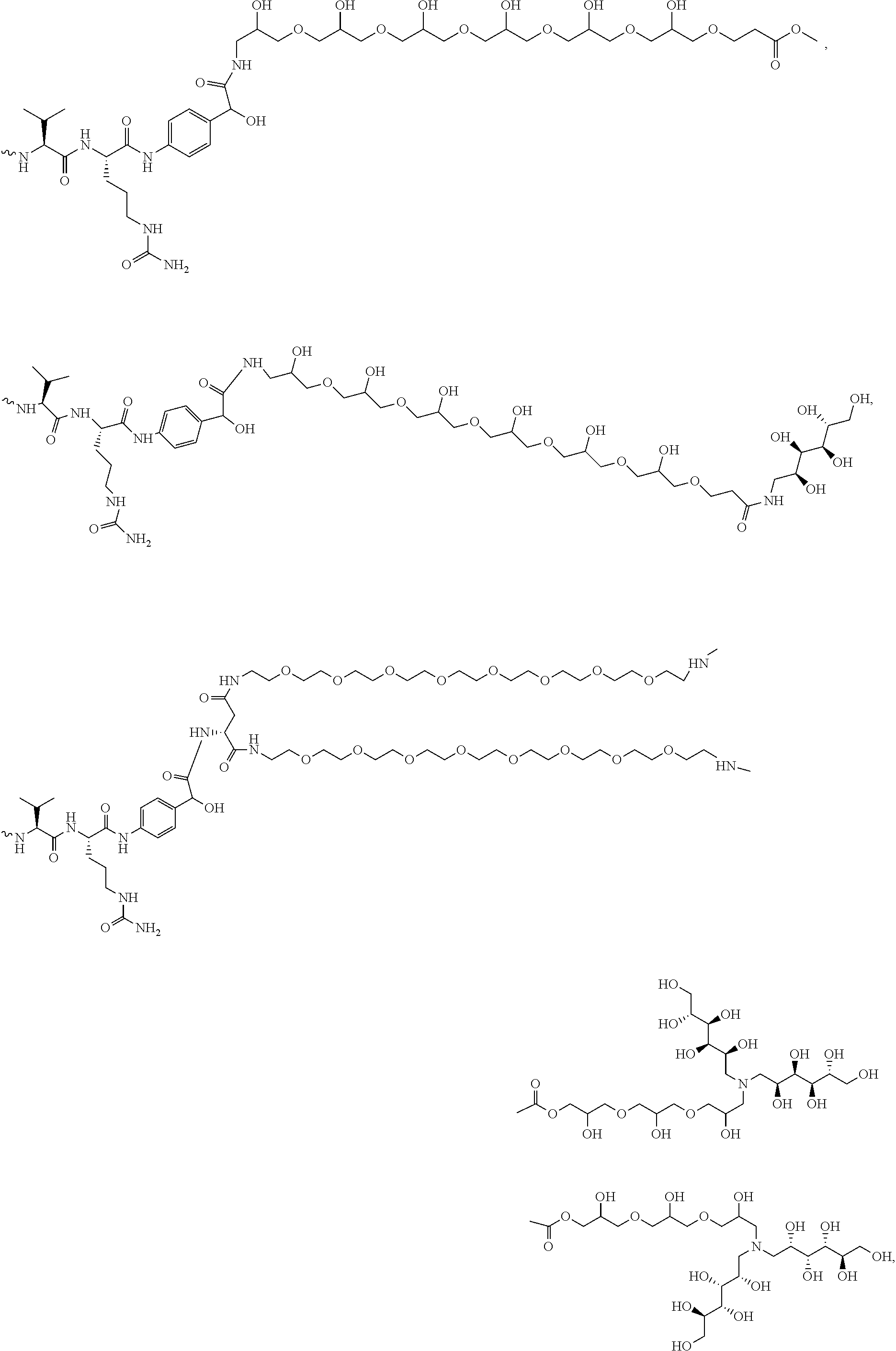 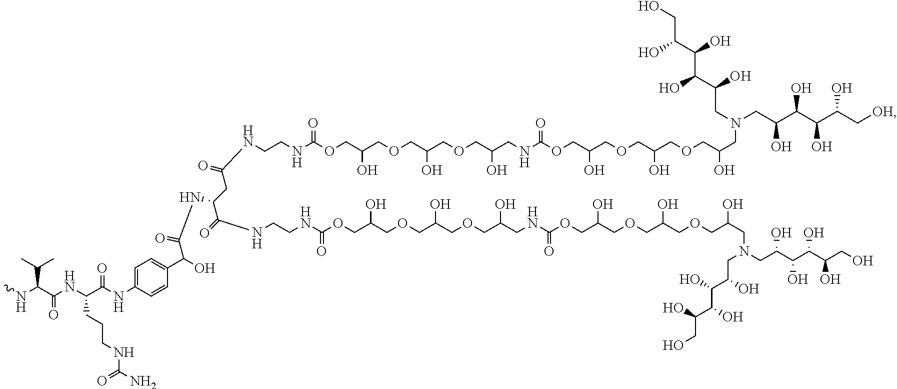 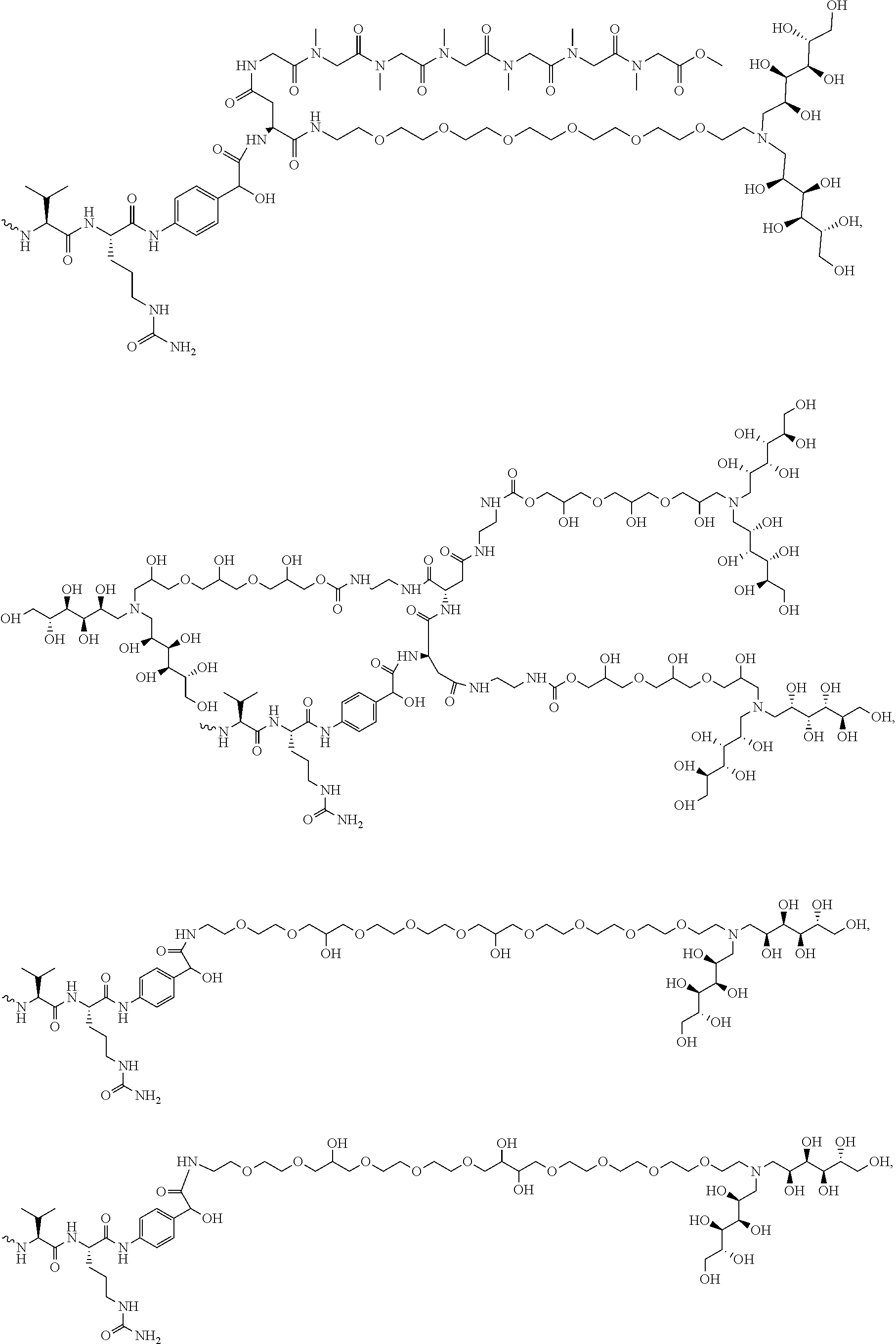 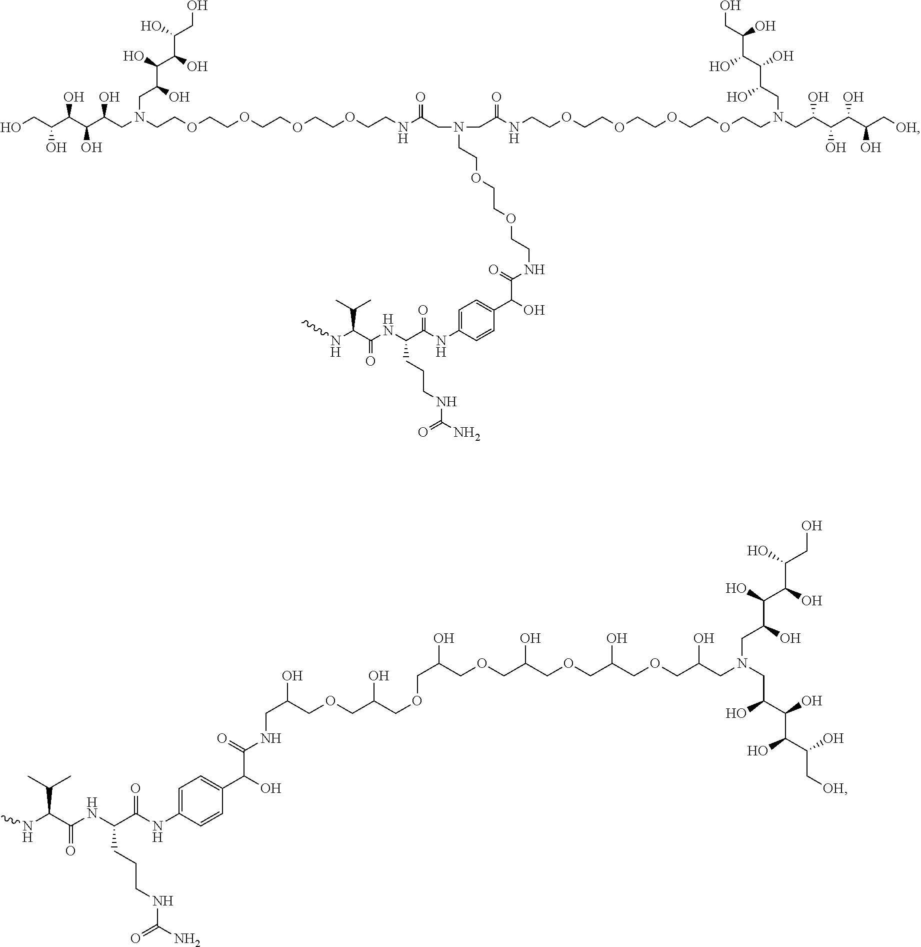 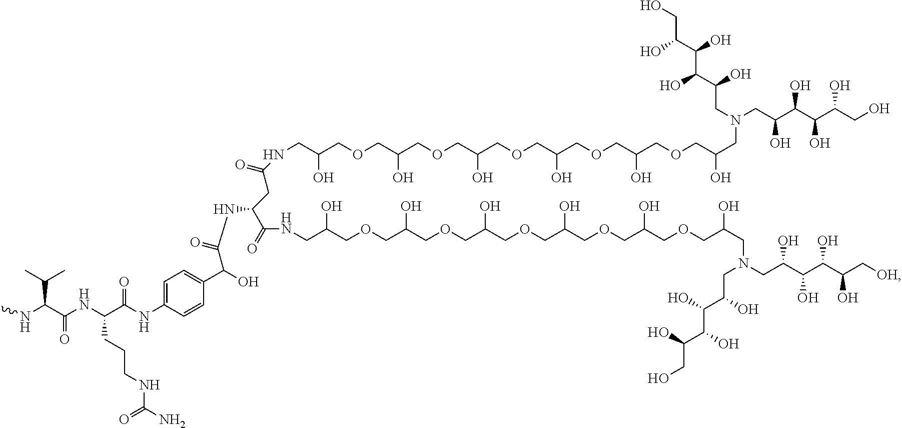
wherein the wavy line on the amino group indicates an attachment site to the stretcher group; or, prior to attachment to the stretcher group, indicates H; and the H on the benzylic OH is optionally replaced with a bond to one of the drug units or a linking group attached to the at least one of the drug units.

69. The conjugate of claim 30, wherein the enzyme-cleavable group is joined to the stretcher group by a non-peptidic linking group.

70. The conjugate of claim 69, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

71. The conjugate of claim 30, comprising the stretcher group attached to the enzyme-cleavable group.

72. The conjugate of claim 71, wherein the stretcher group is selected from the following: 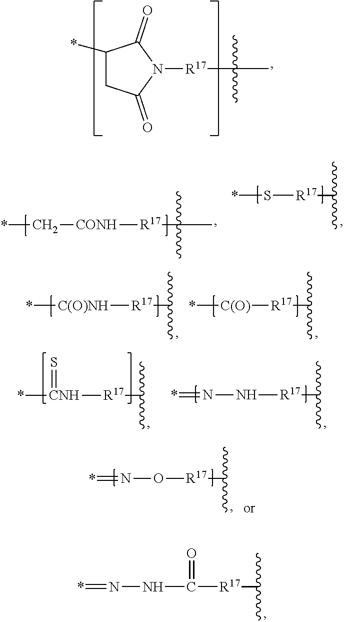
wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈ alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₅ alkylene-(C(═O))—NH—(CH₂—O—CH₂)_{b}—C₁-C₈ alkylene-C(═O)— (where b is 1 to 26), —C₁-C₅ alkylene-NH—(C(═O))—(CH₂—O—CH₂)_{b}—NH— (where b is 1 to 26), —C₁-C₅ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₅ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀ heteroalkylene-S—, —C₃-C₈ carbocyclo-S—, —O—(C₁-C₅ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the stretcher group comprises maleimido(C₁-C₁₀alkylene-C(O)—, maleimido(CH₂OCH₂)ₚ₂(C₁-C₁₀alkylene)C(O)—, maleimido(C₁-C₁₀alkylene) (CH₂OCH₂)ₚ₂C(O)—, or a ring open form thereof, wherein p2 is from 1 to 26;
and wherein * is an attachment to the binding agent, and the wavy line is an attachment to the enzyme-cleavable group.

73. The conjugate of claim 72, wherein the stretcher group is derived from a structure selected from the following: 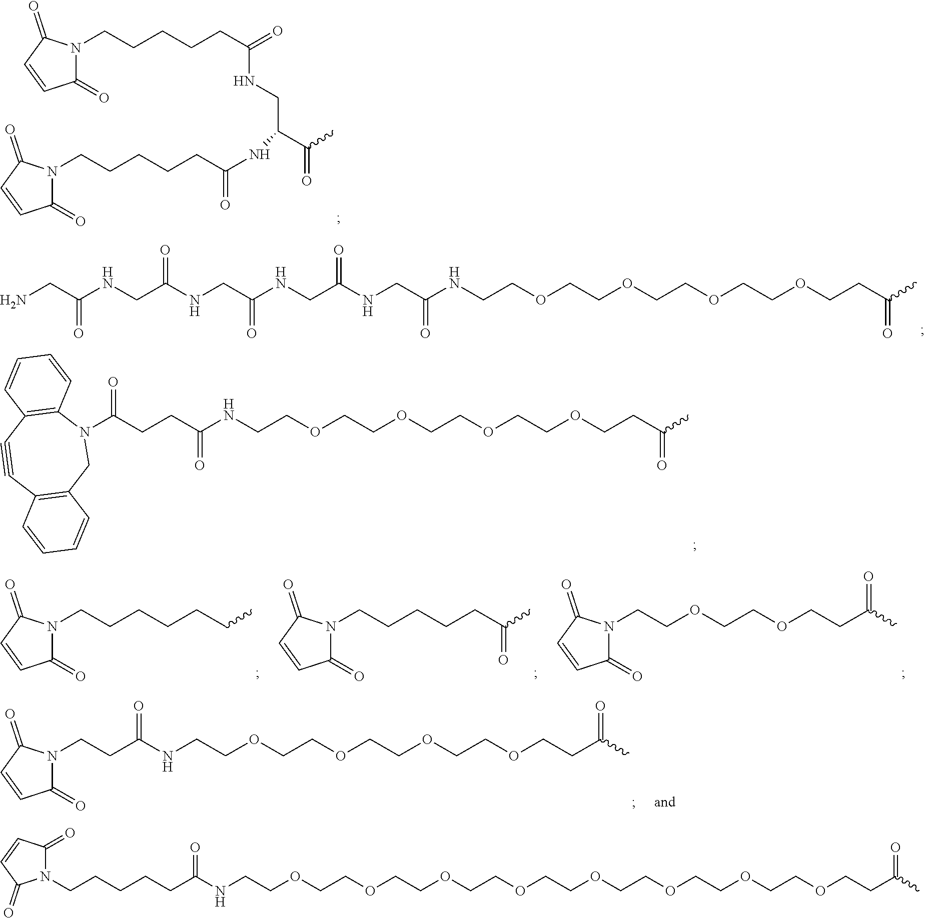
wherein the wavy line [symbol] indicates an attachment site of the stretcher group to the enzyme-cleavable group, and the attachment site to the binding agent is on the maleimide, primary amine or alkyne functional group.

74. The conjugate of claim 26, wherein the linker compound has one of the following structures: 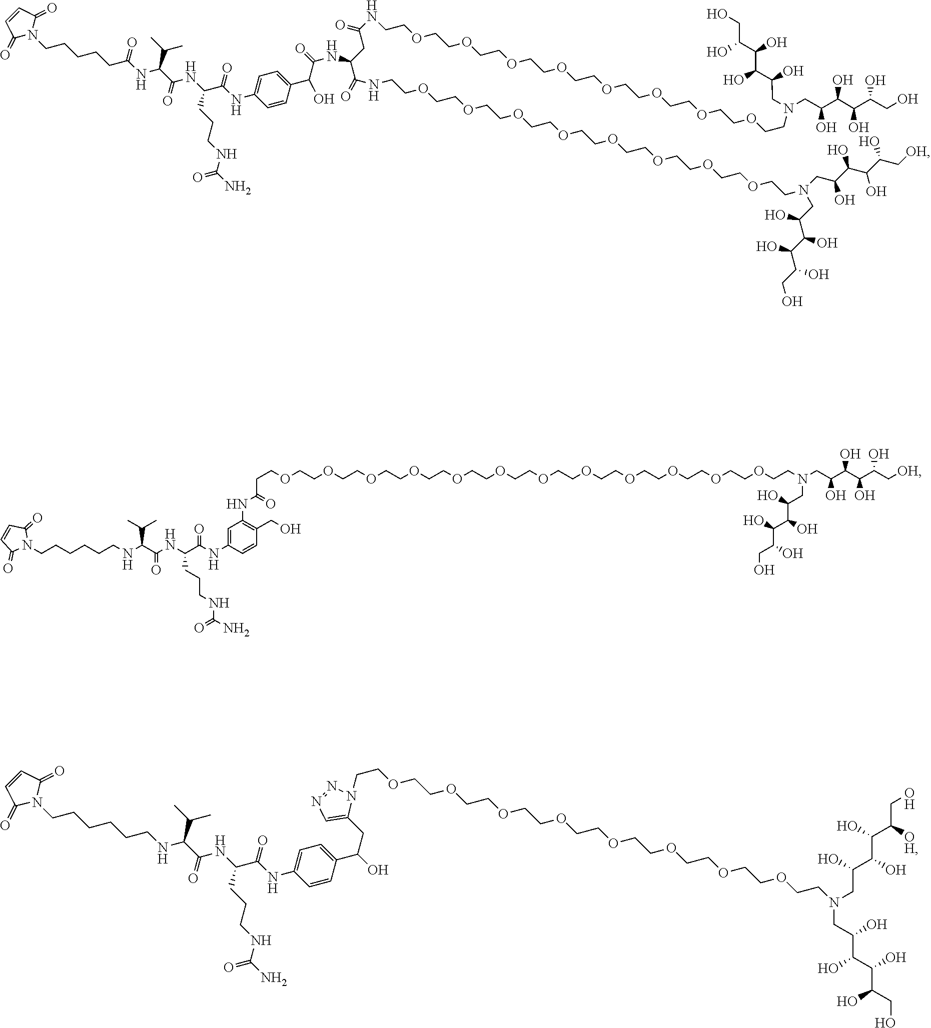 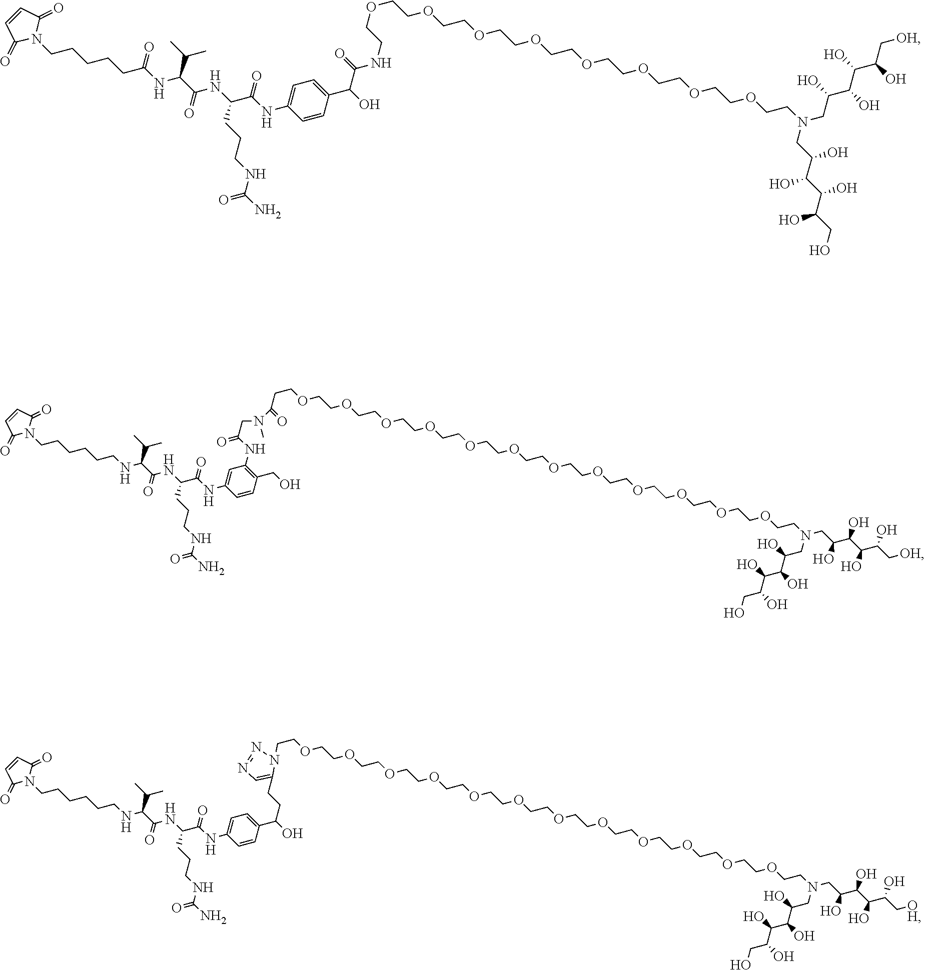 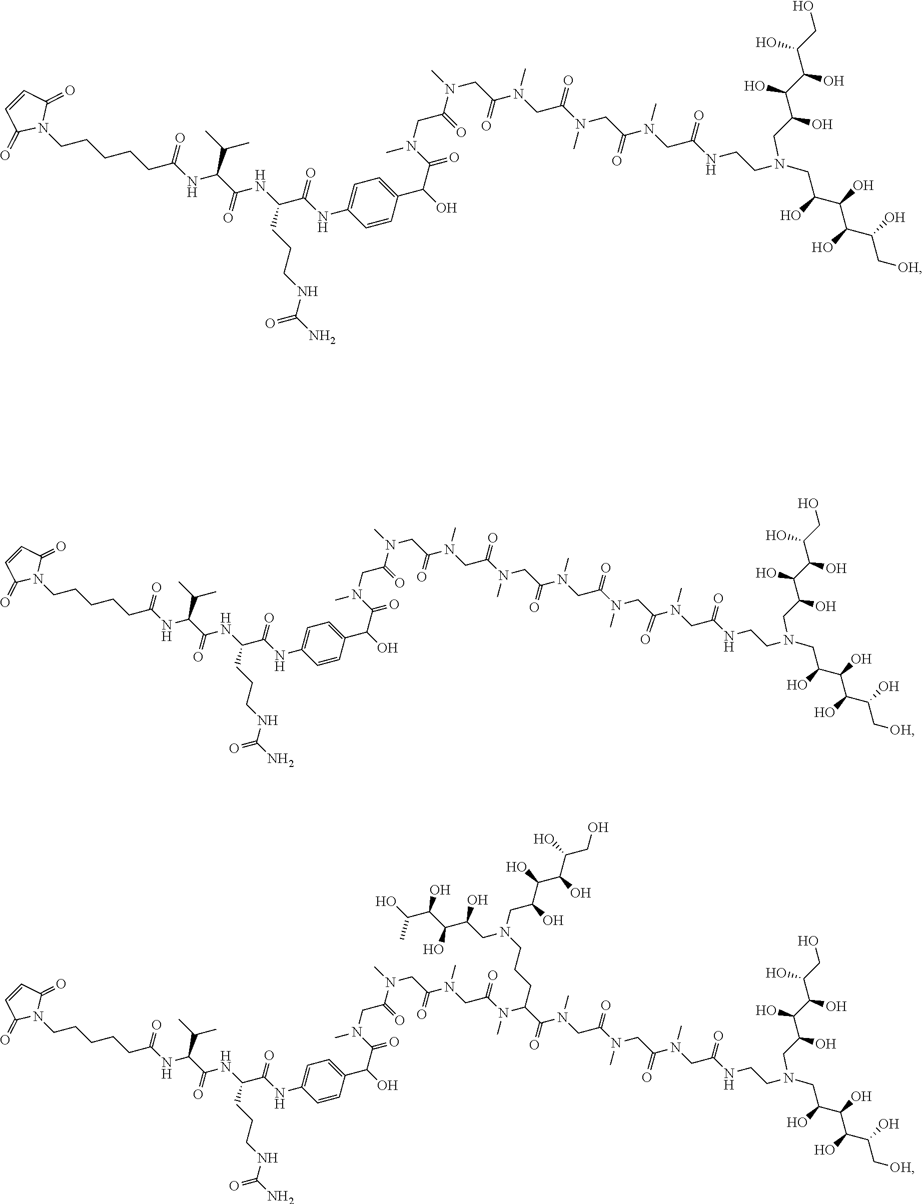 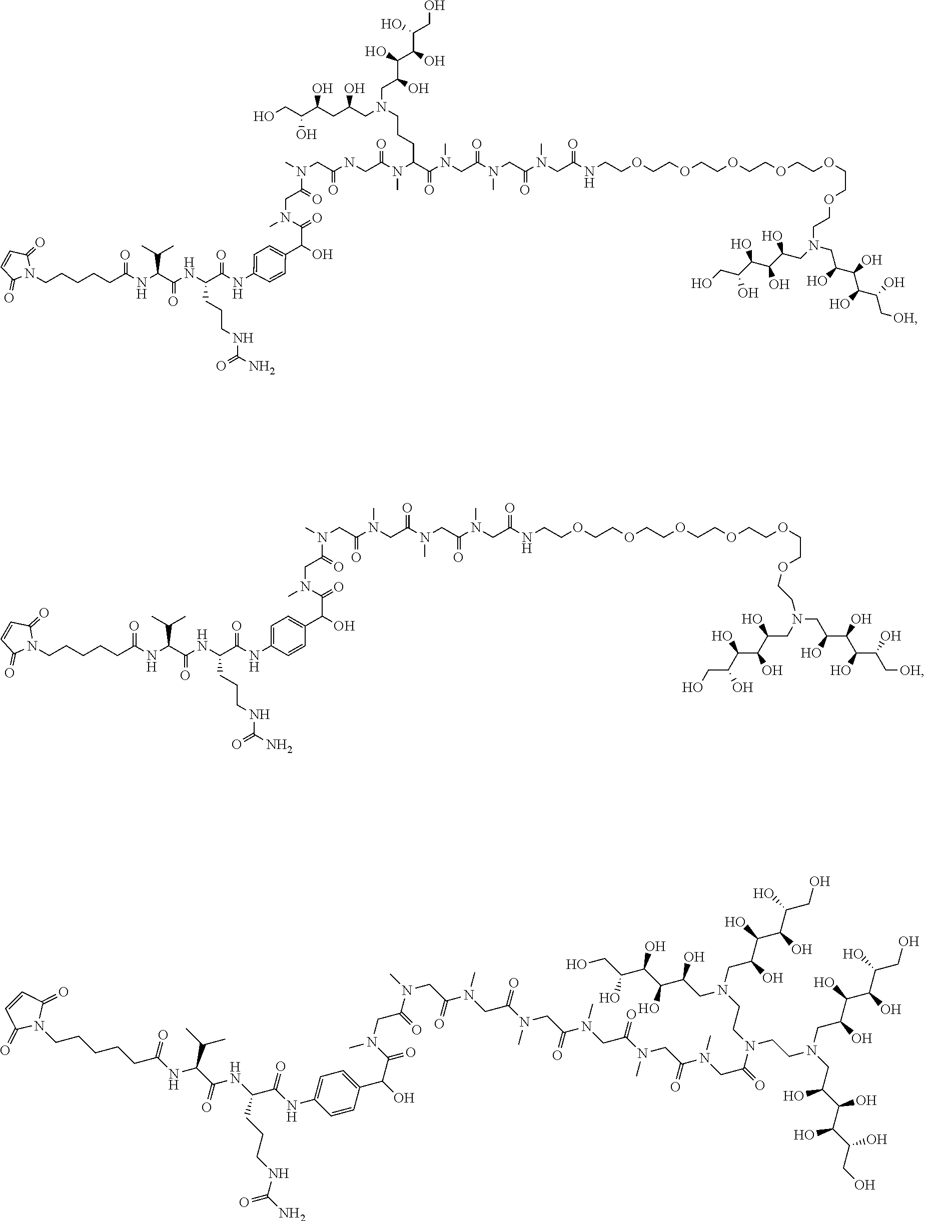 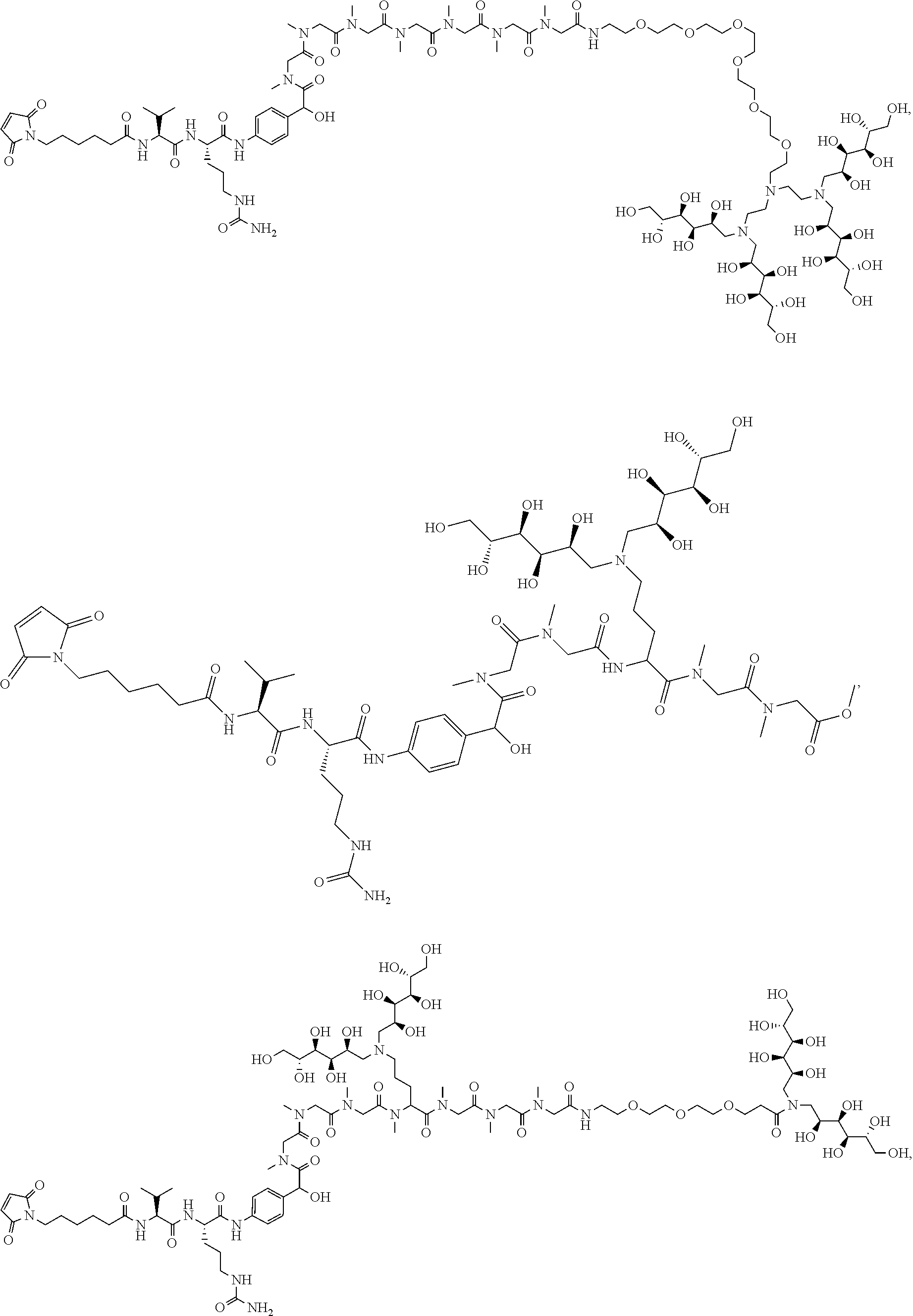 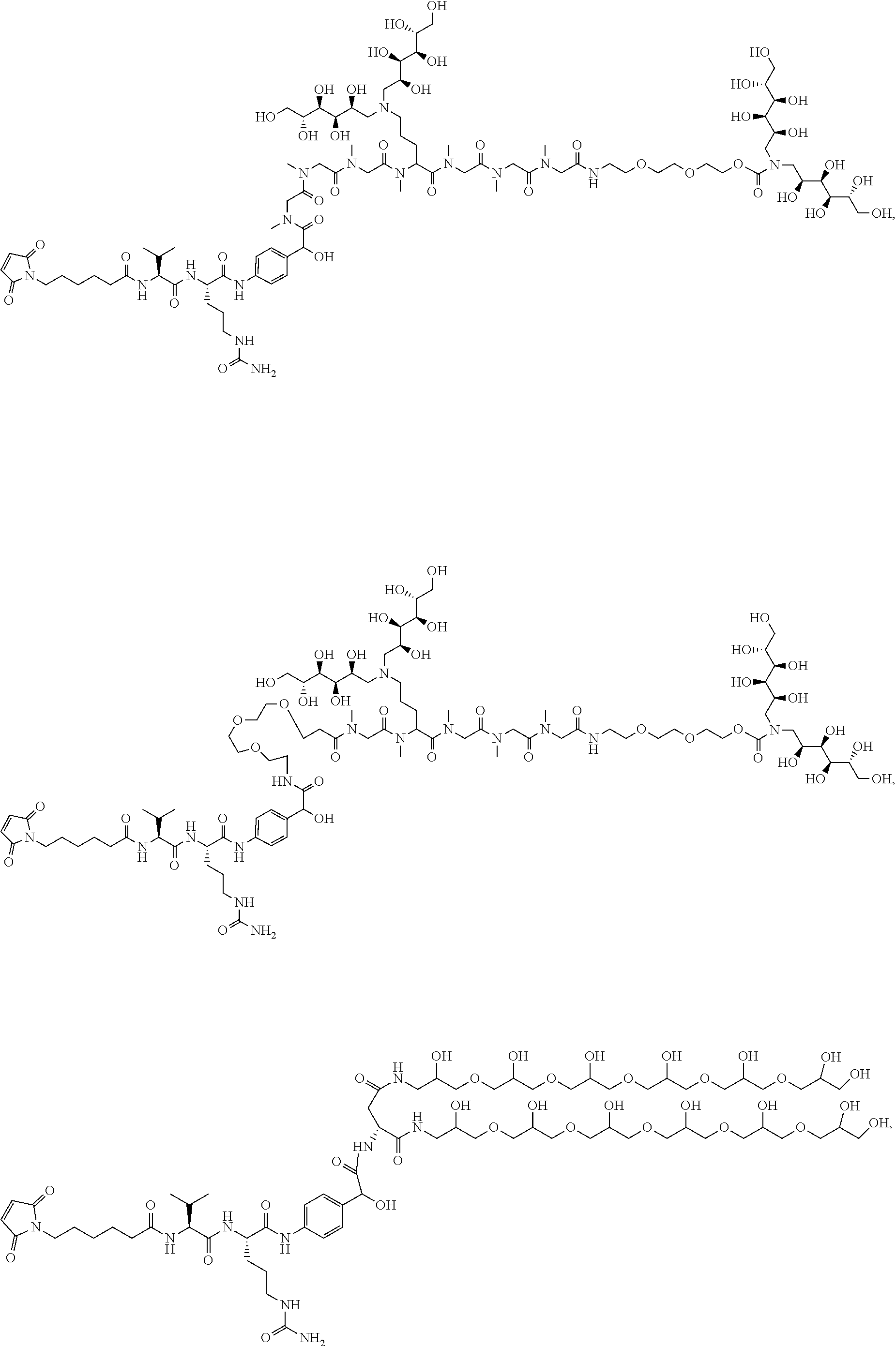 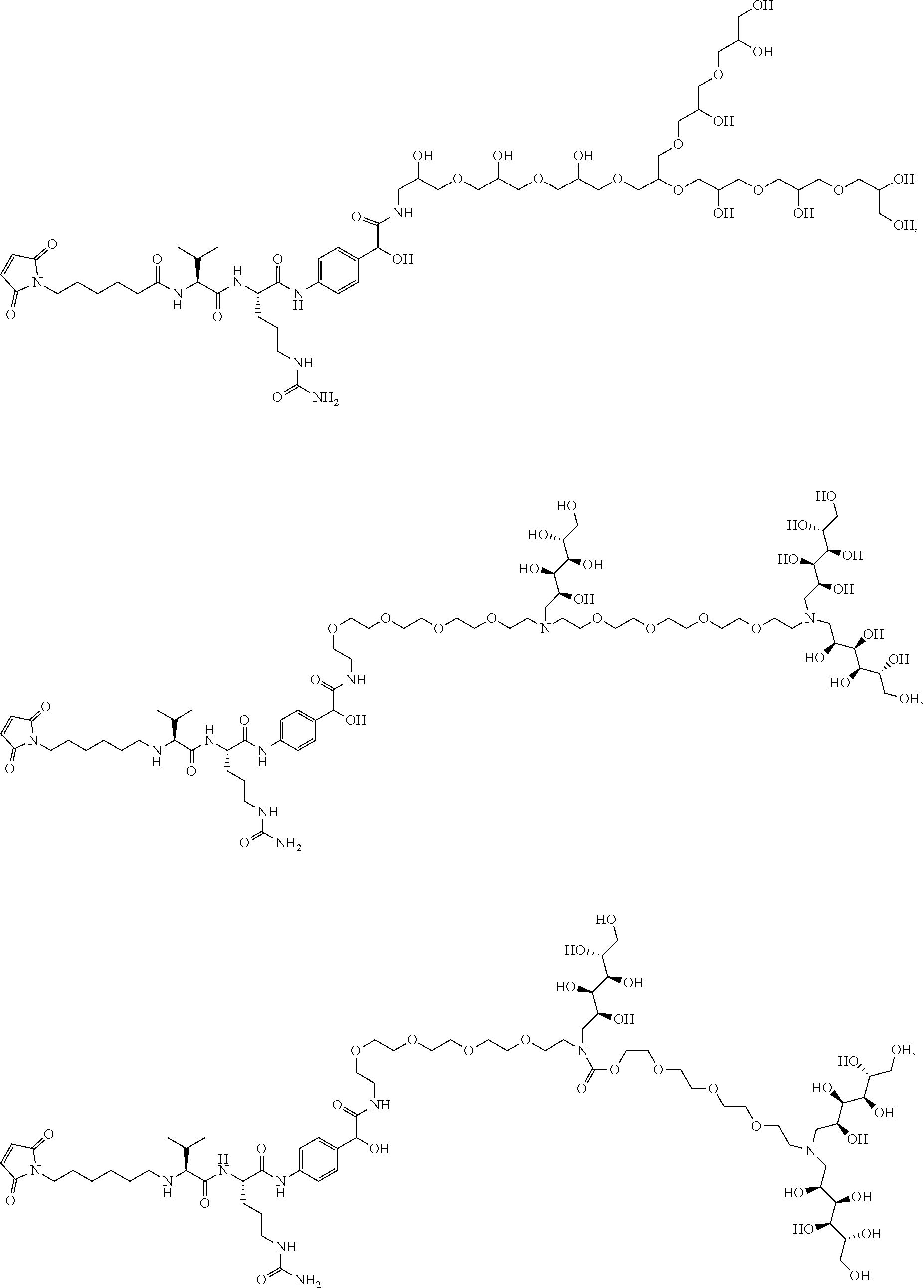 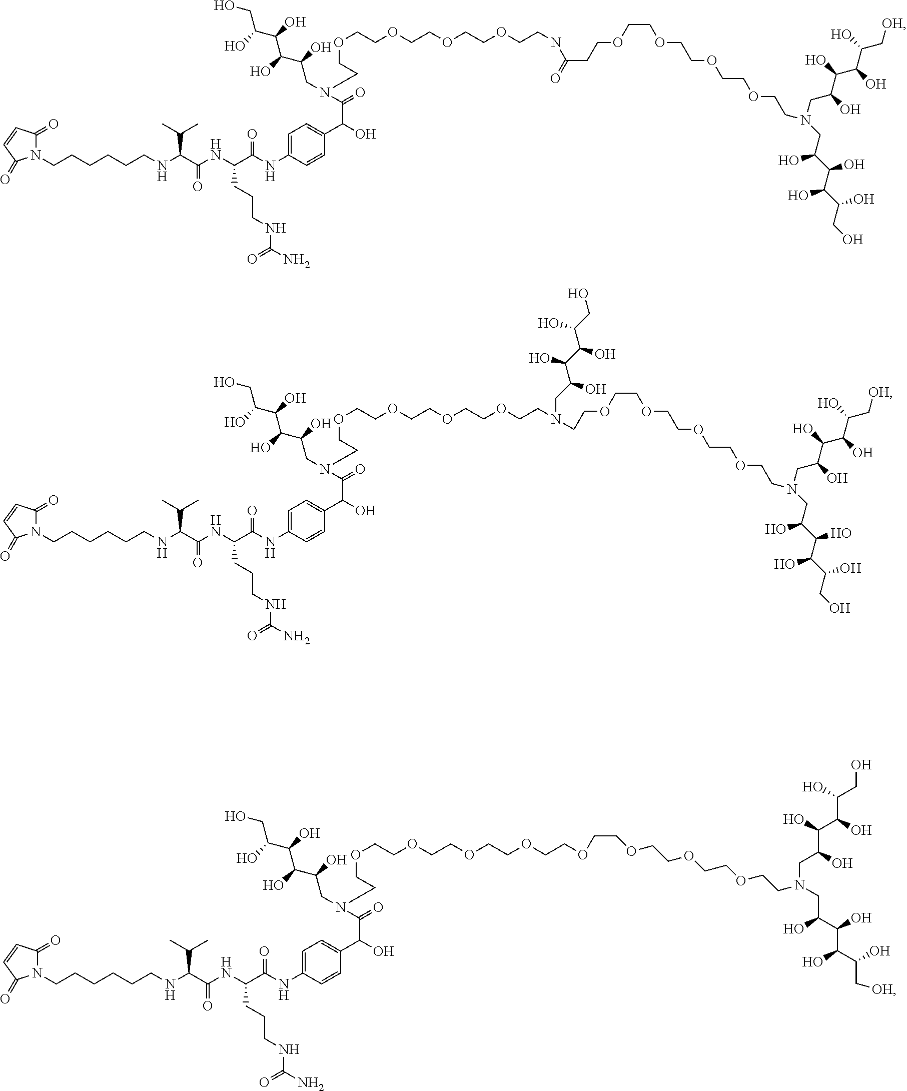 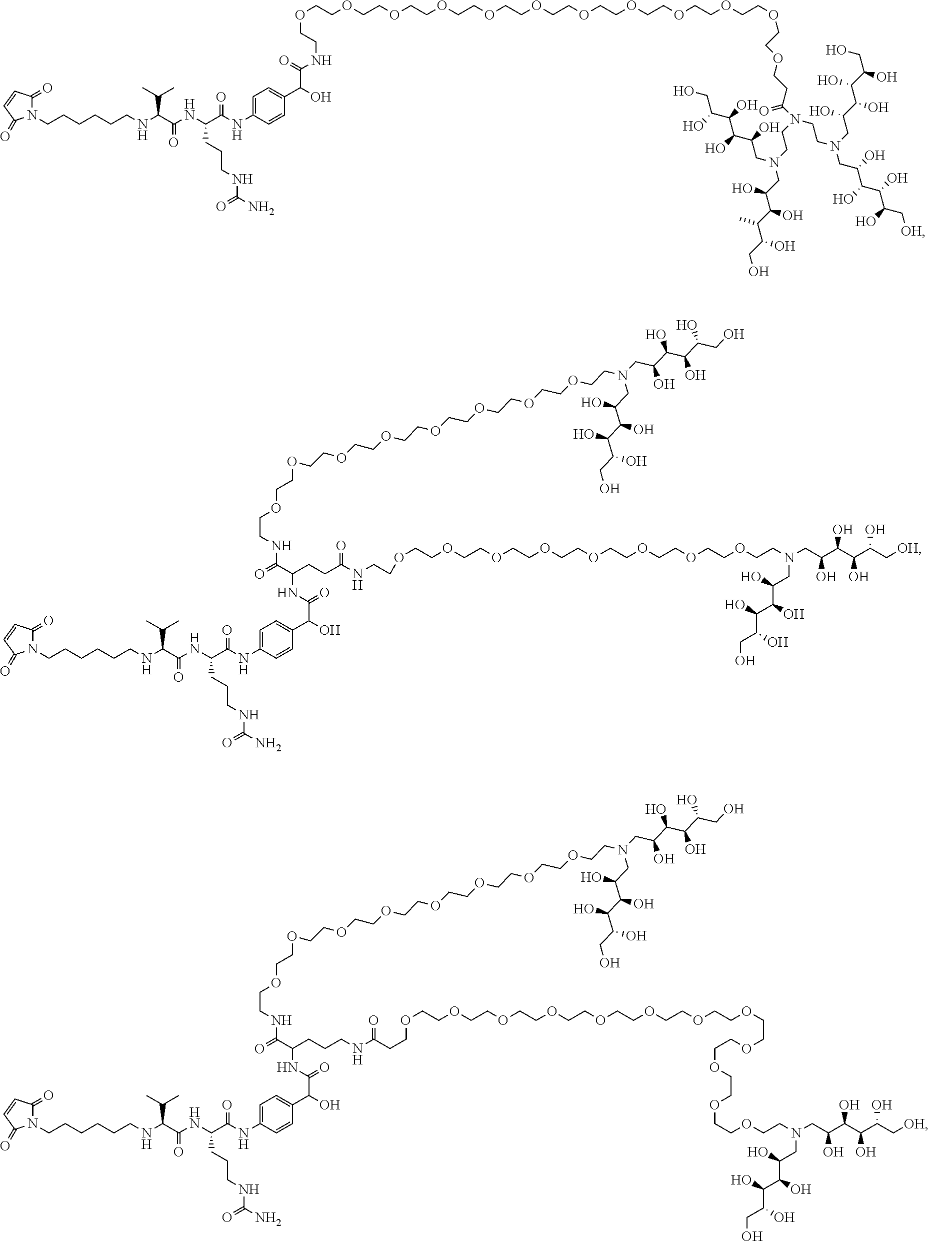 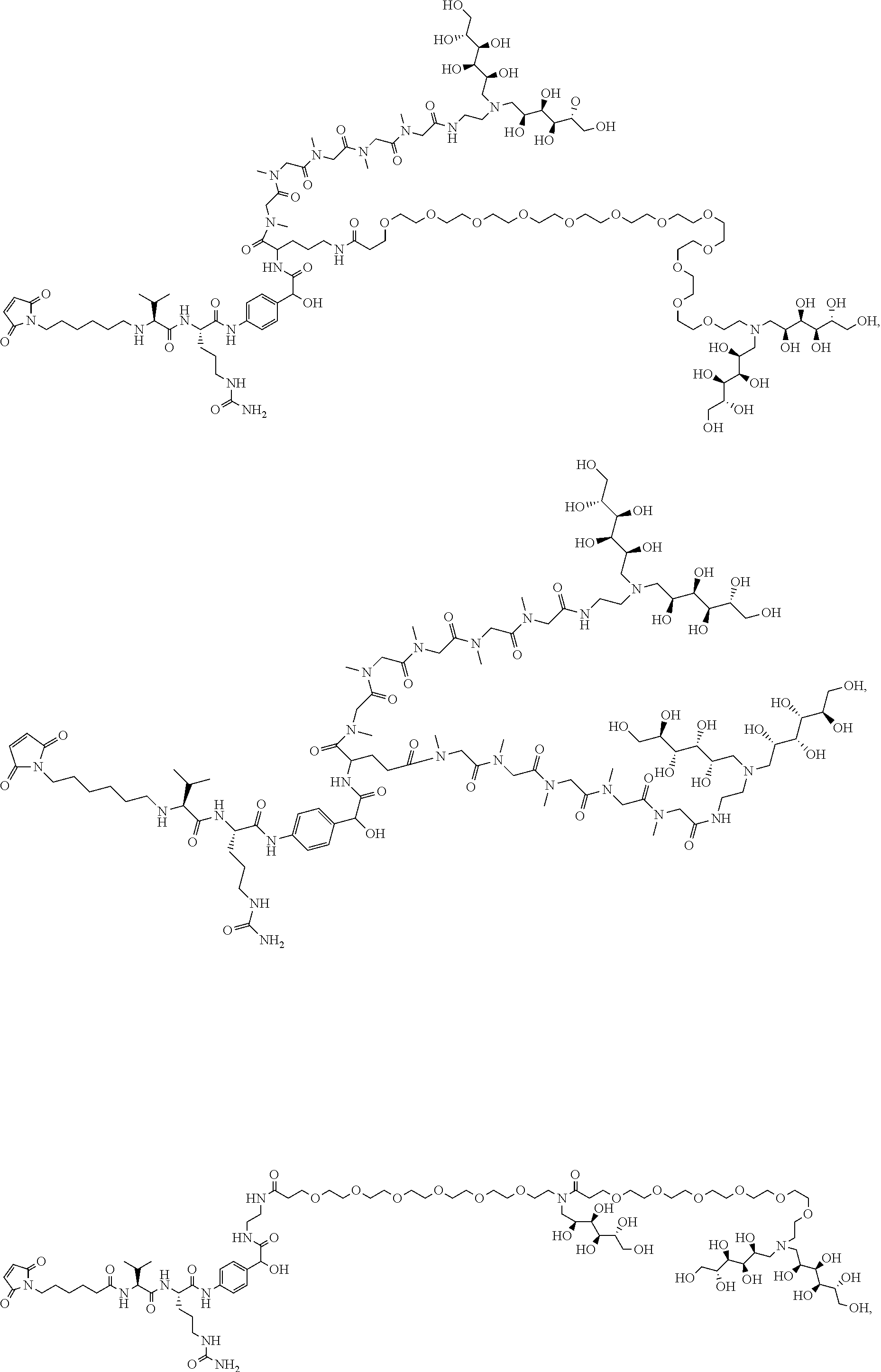 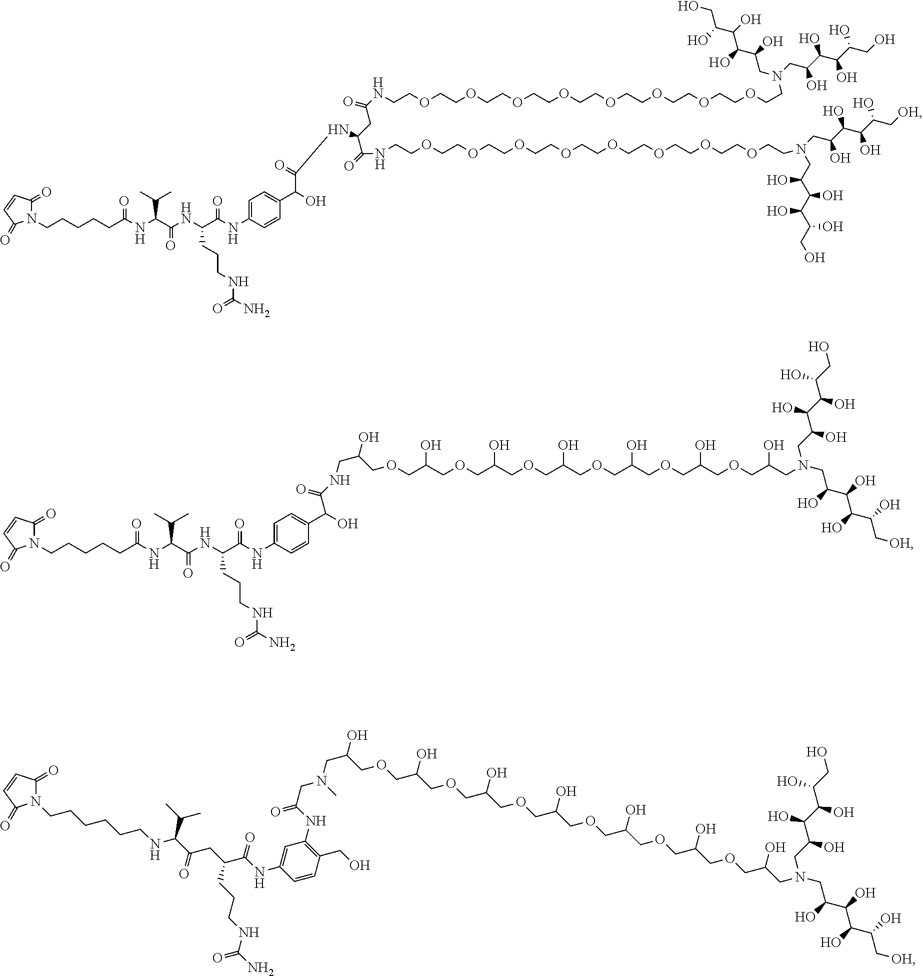 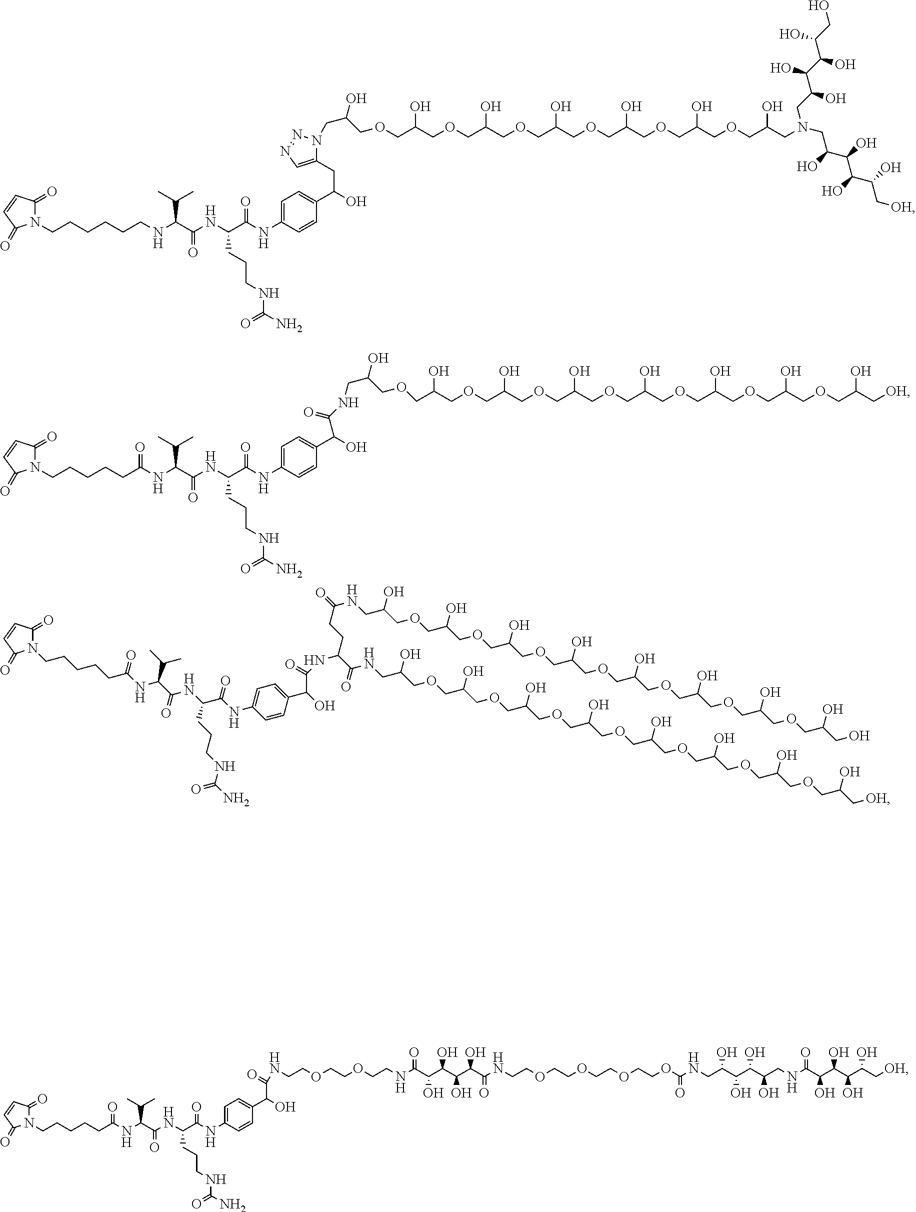 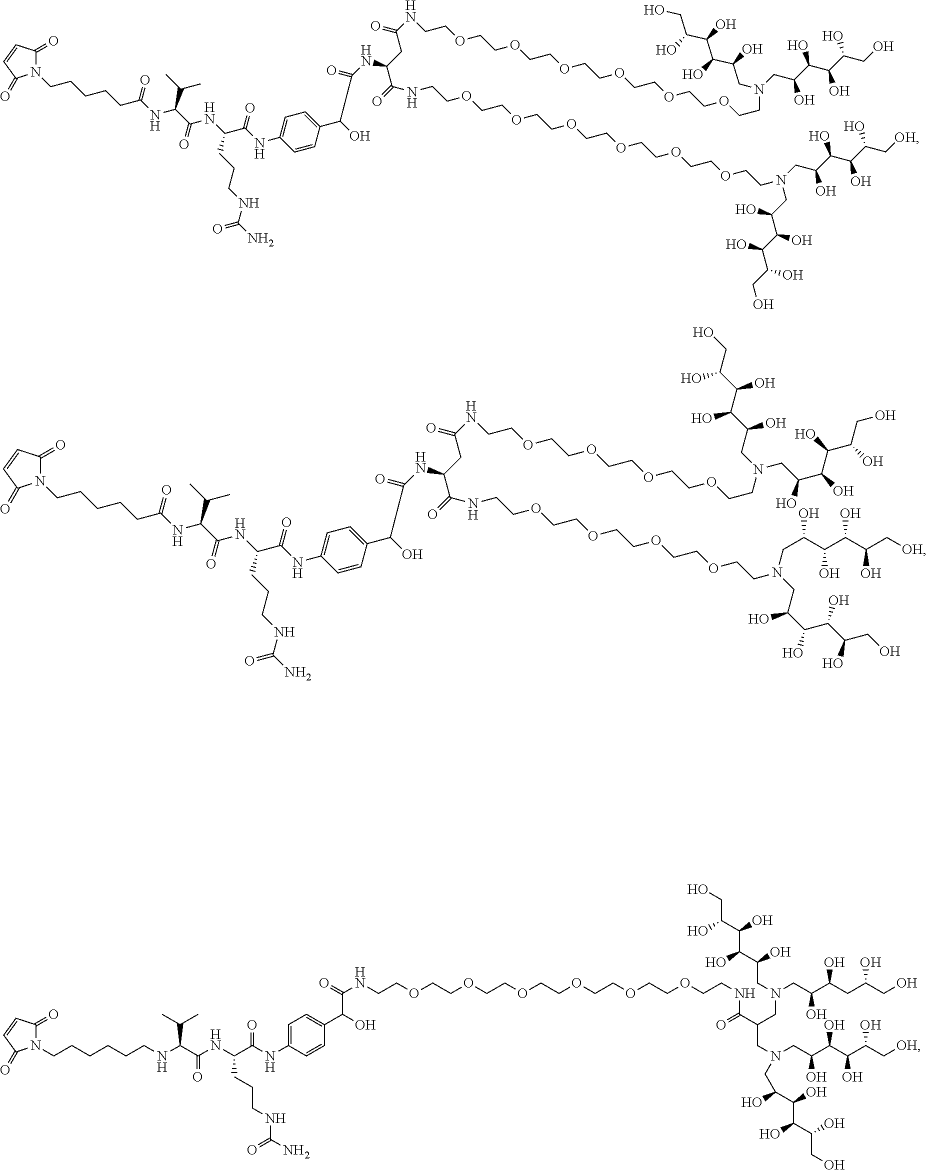 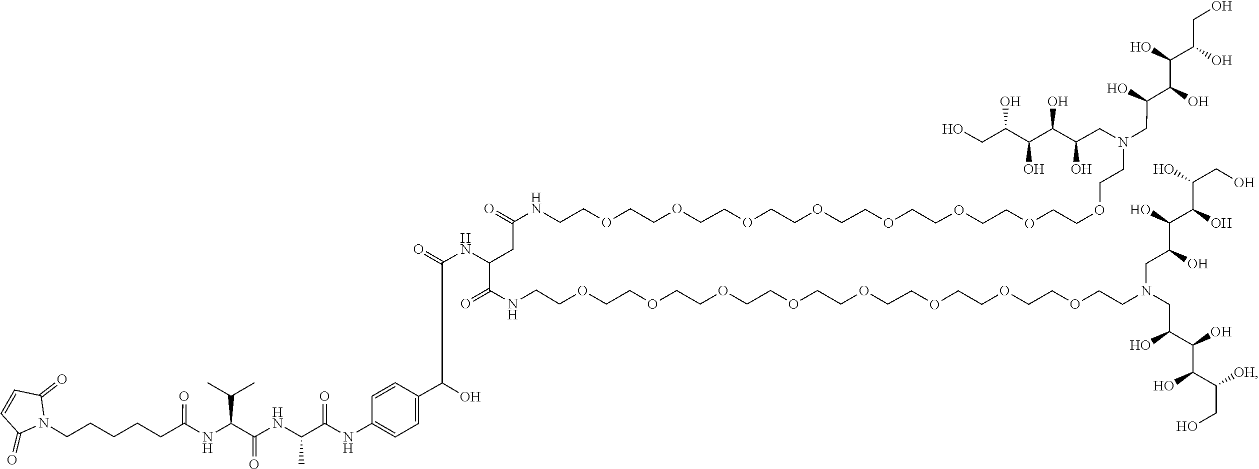 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 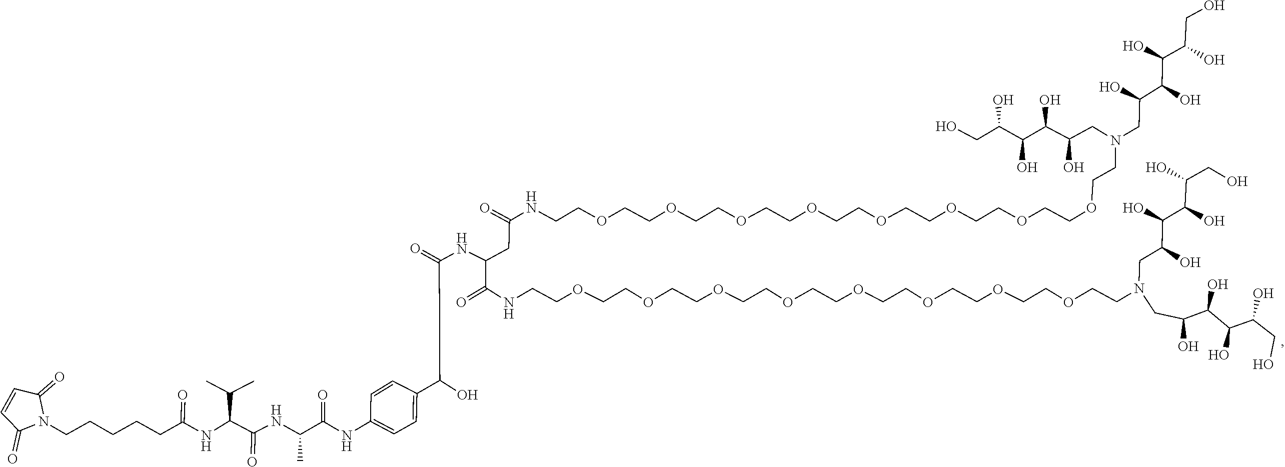 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 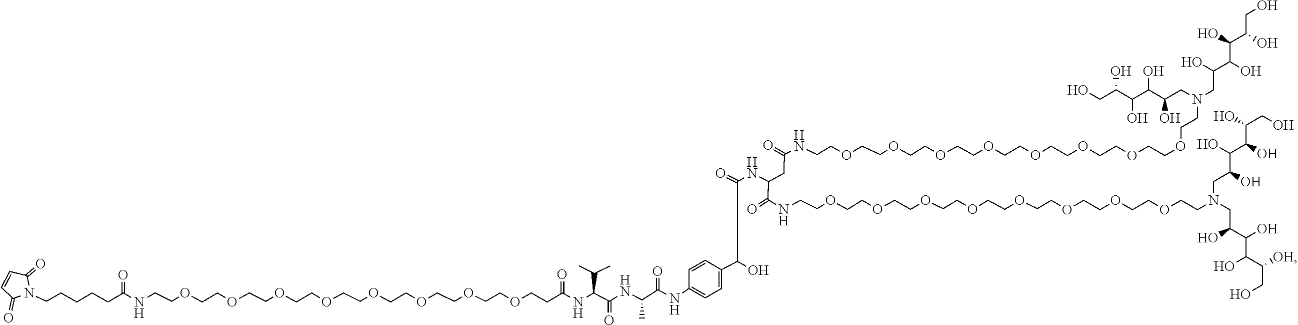 [SMILES: CC(C)[C@H](NC(=O)CCOCCOCCOCCOCCOCCOCCOCCOCCNC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(CC(O)C(O)[C@@H](O)[C@@H](O)CO)C[C@@H](O)C(O)C(O)[C@@H](O)CO)C(=O)NCCOCCOCCOCCOCCOCCOCCOCCOCCN(C[C@H](O)C(O)C(O)[C@H](O)CO)C[C@H](O)C(O)[C@H](O)[C@H](O)CO)cc1] 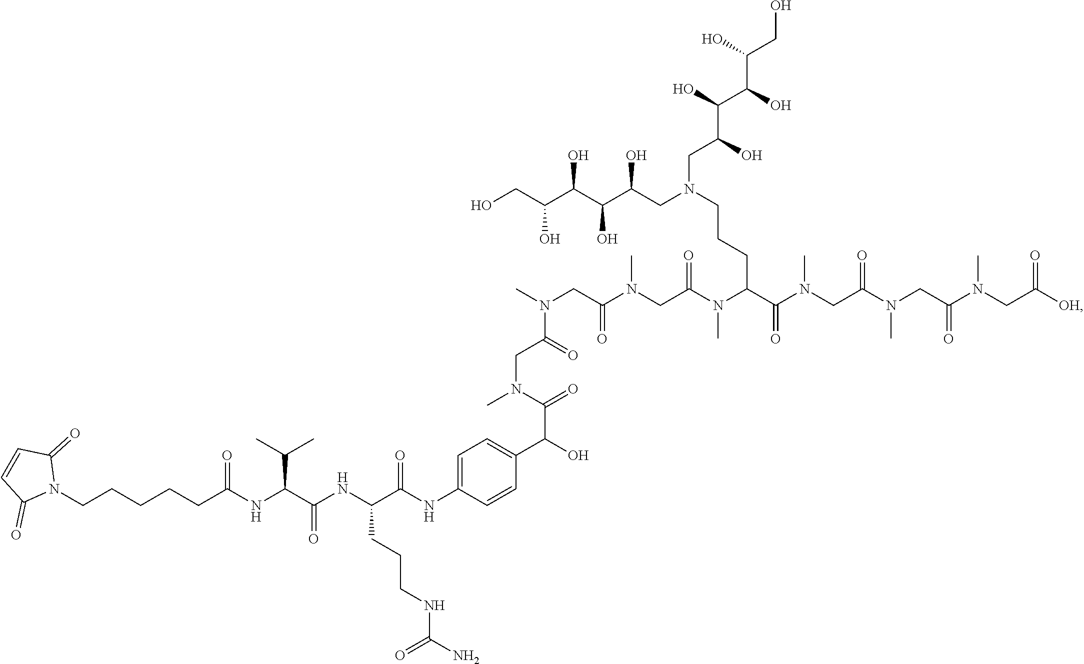 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 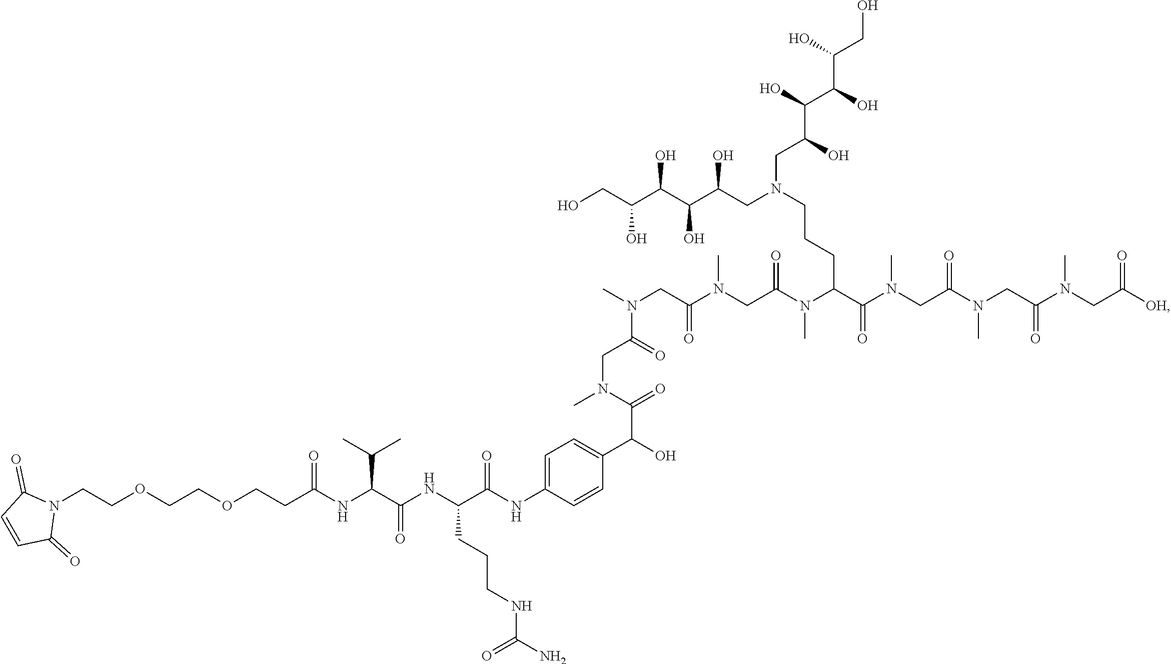 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 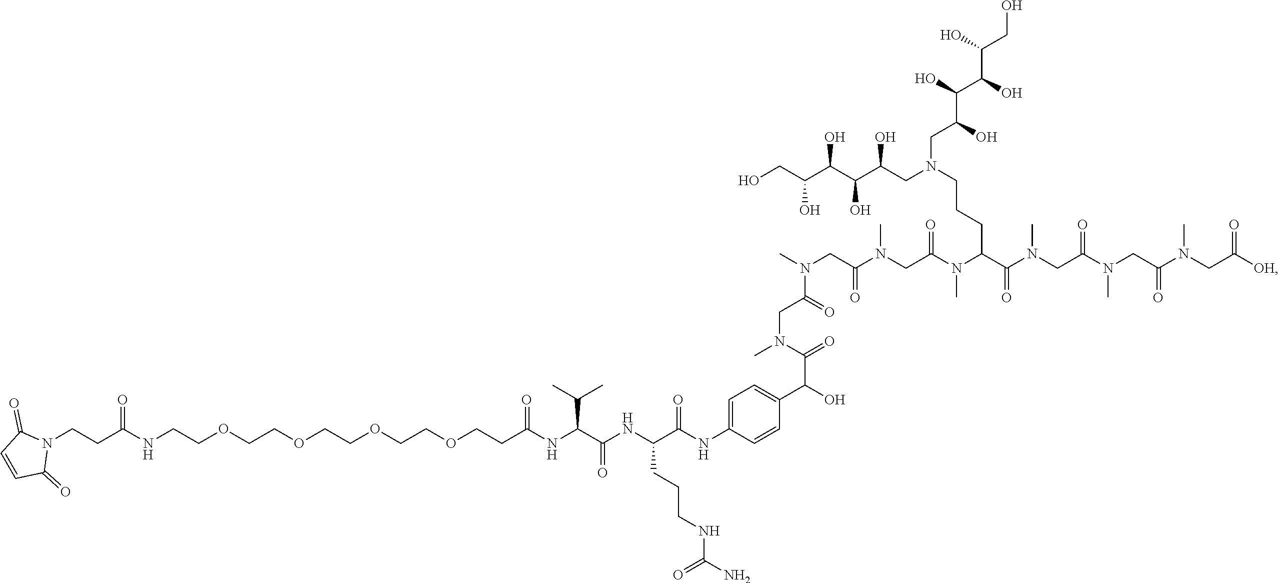 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 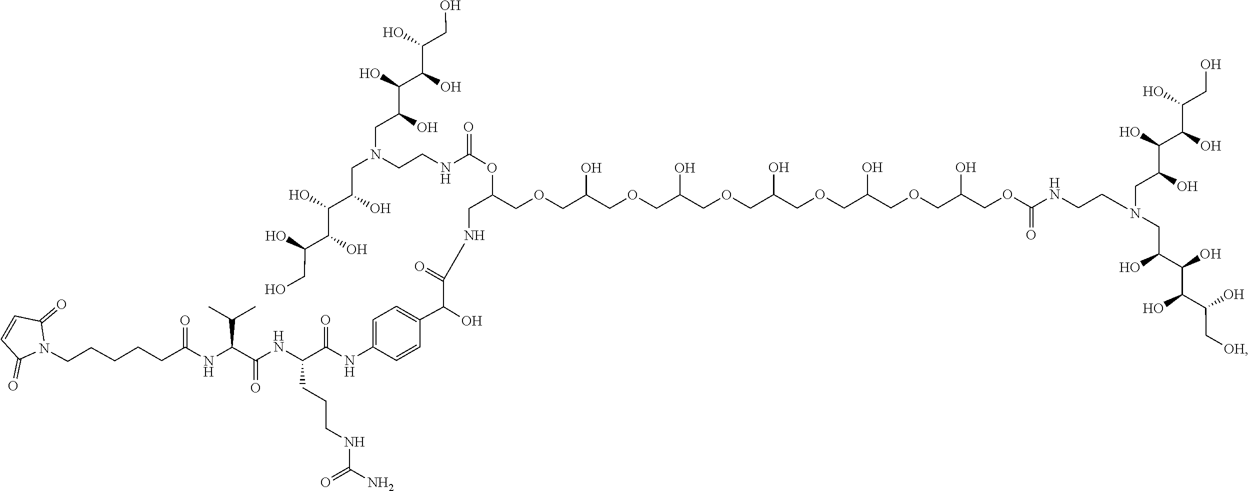 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 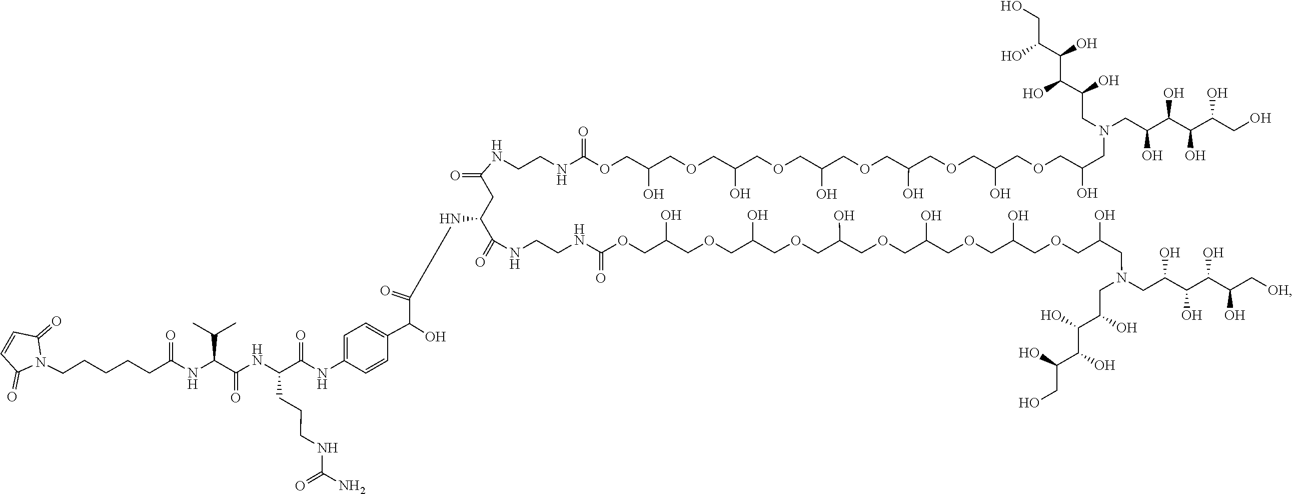 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 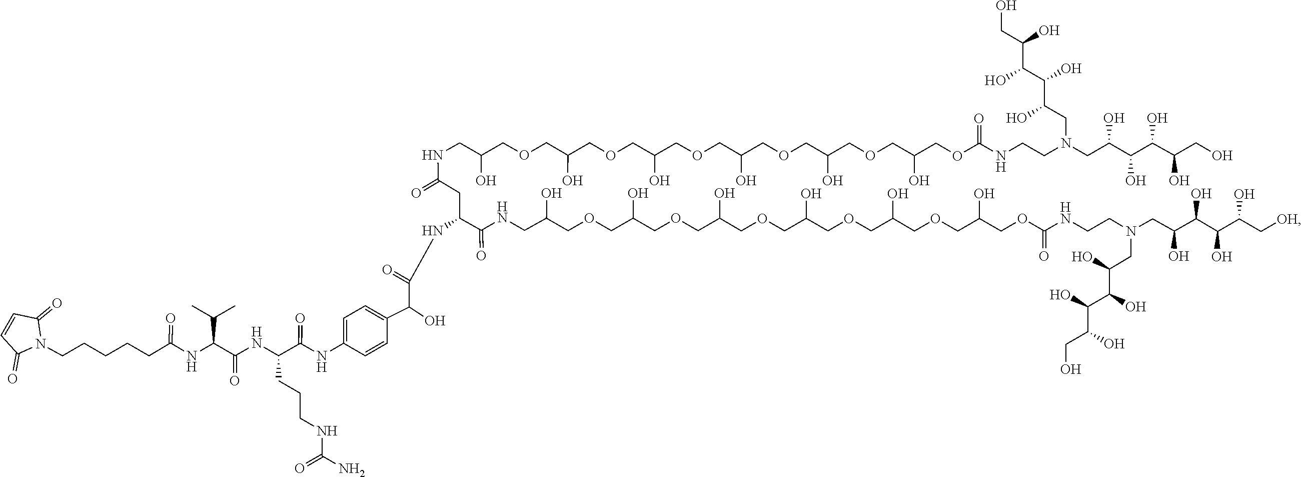 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1] 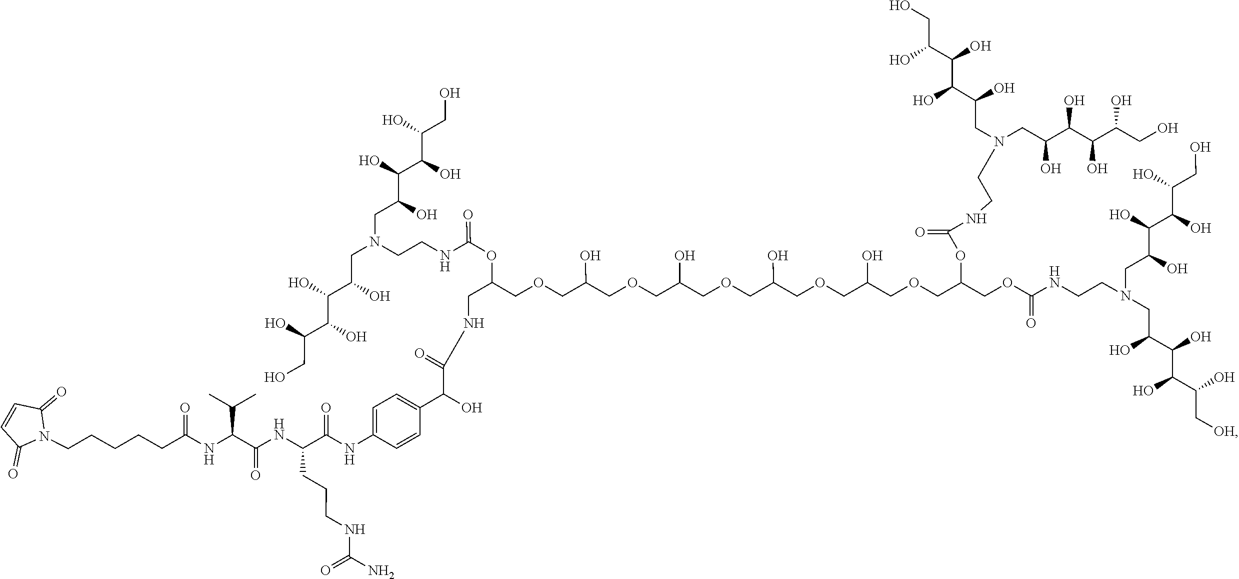 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 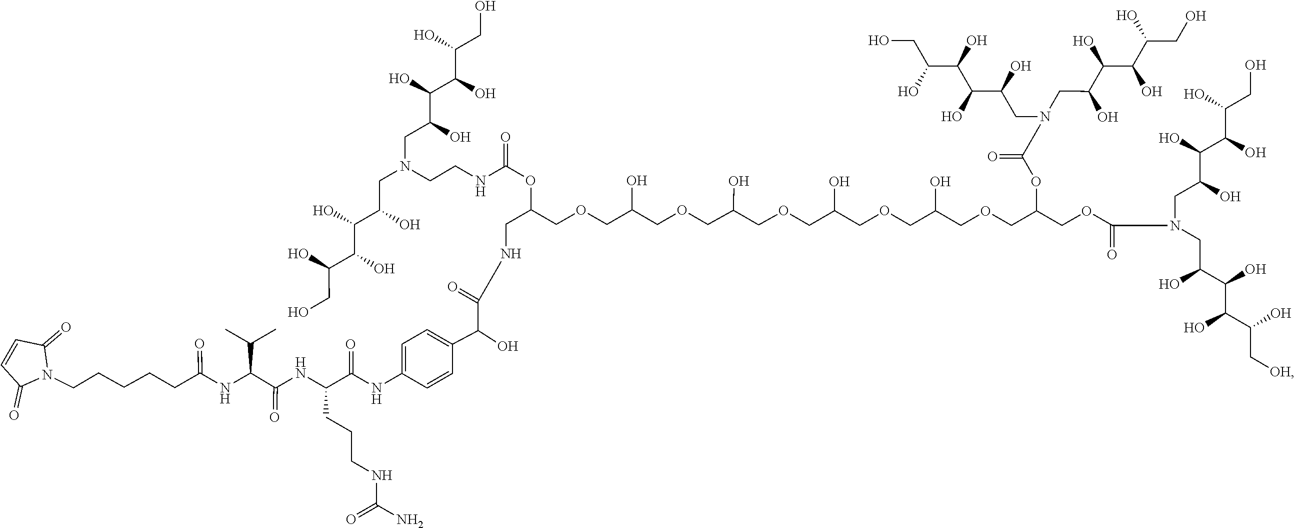 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)COCC(O)COCC(O)COCC(O)COCC(COC(=O)N(C[C@H](O)[C@@H](O)[C@H](O)[C@H](O)CO)C[C@H](O)[C@@H](O)[C@H](O)[C@H](O)CO)OC(=O)N(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)cc1] 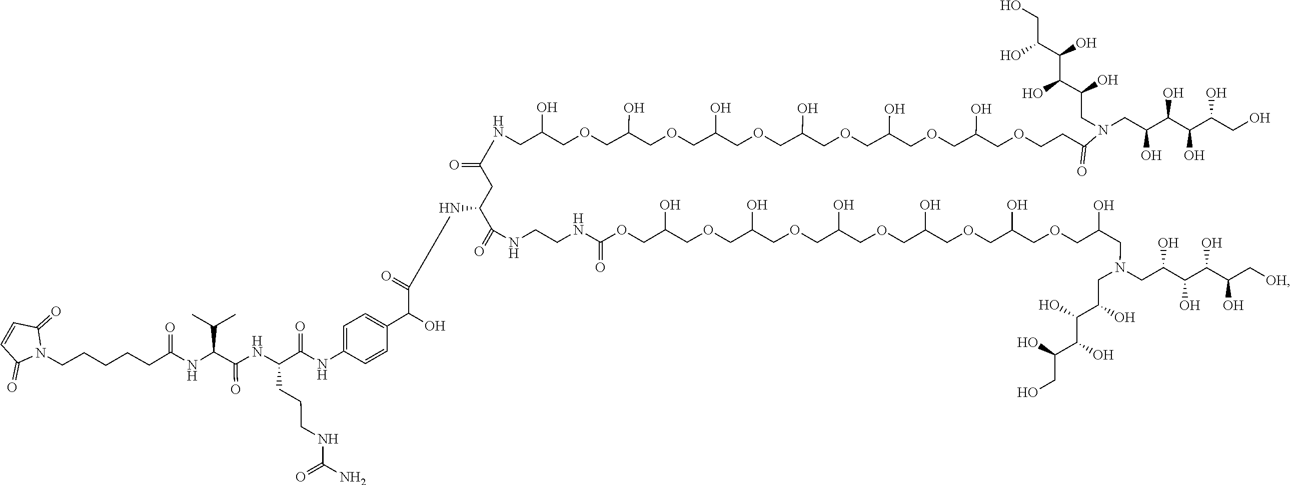 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COCCC(=O)N(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 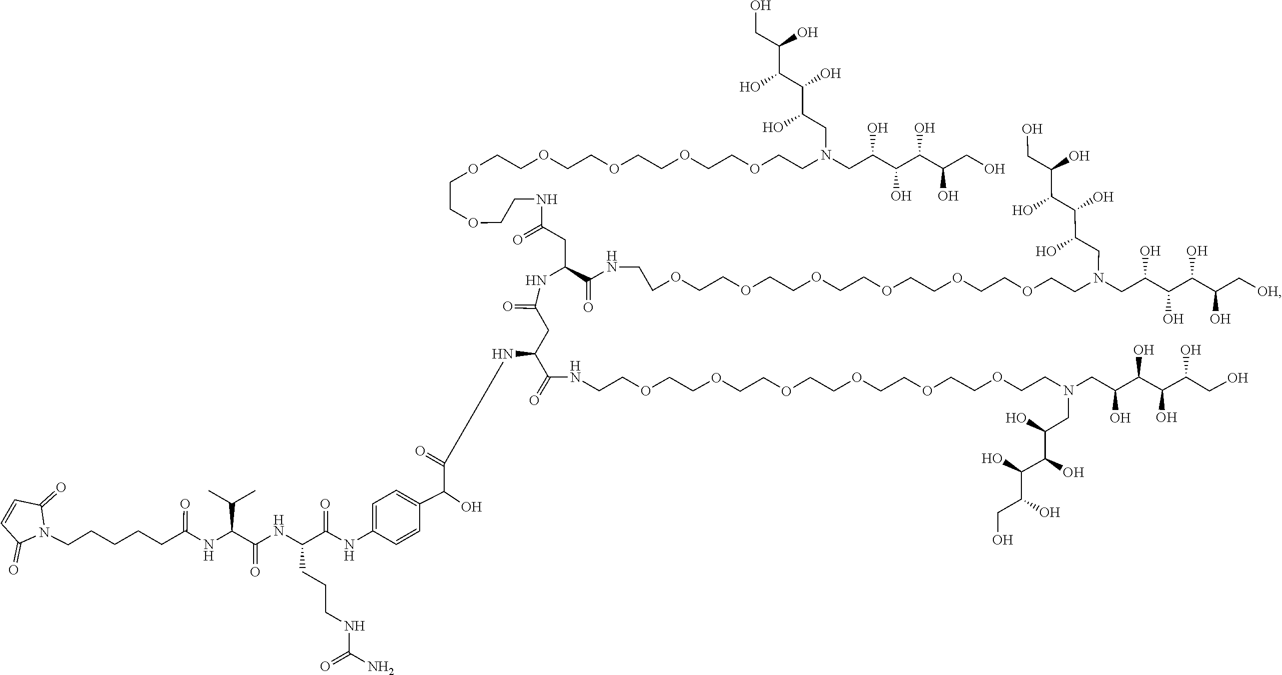 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 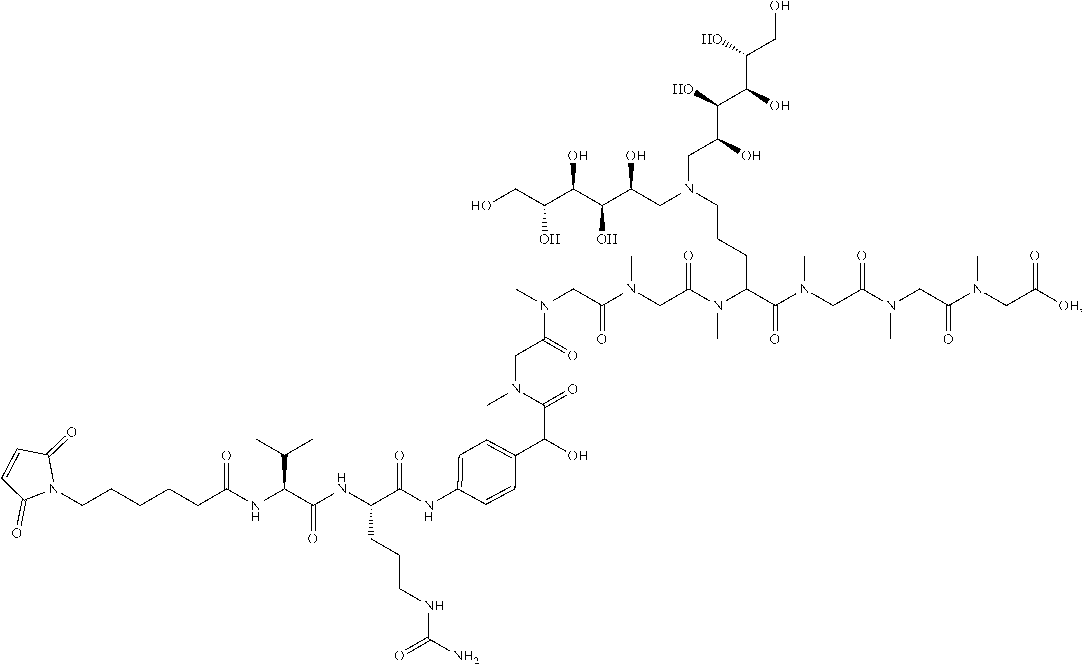 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 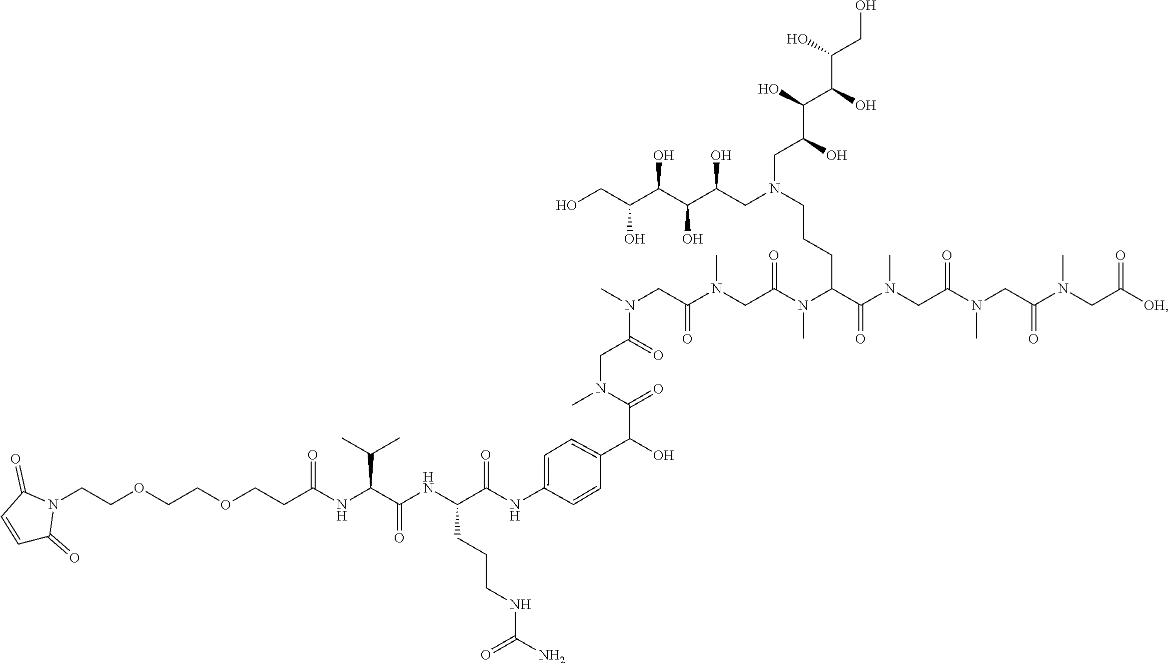 [SMILES: CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1] 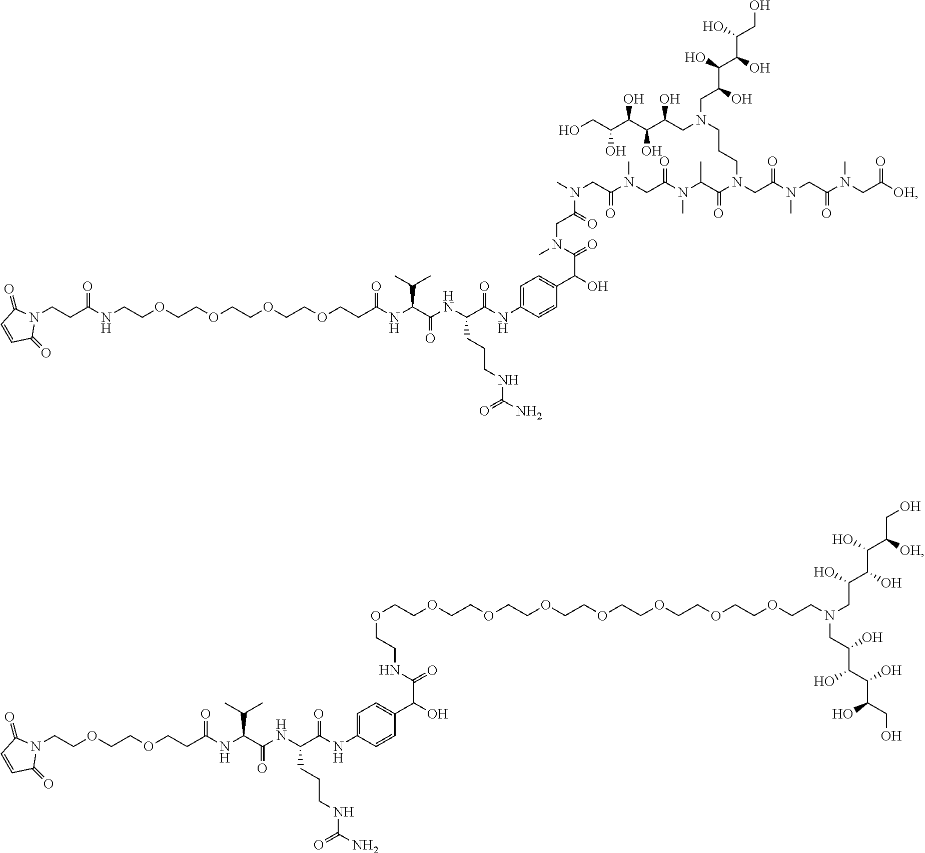 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(C)C(=O)N(CCCN(C[C@H](O)[C@@H](O)[C@H](O)[C@H](O)CO)C[C@H](O)[C@@H](O)[C@H](O)[C@H](O)CO)CC(=O)N(C)CC(=O)N(C)CC(=O)O)cc1 ; CC(C)[C@H](NC(=O)CCOCCOCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1] 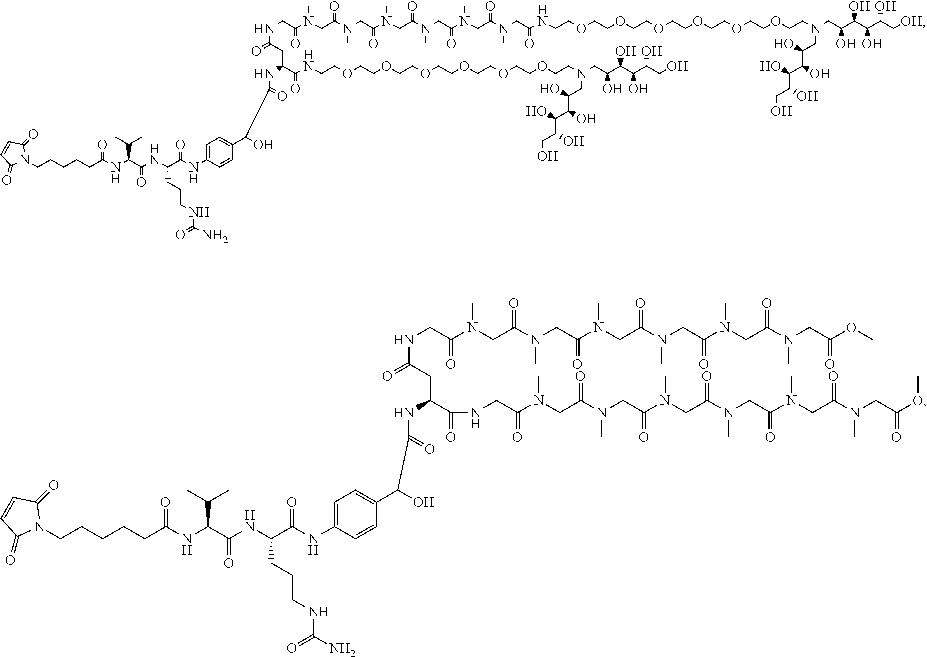 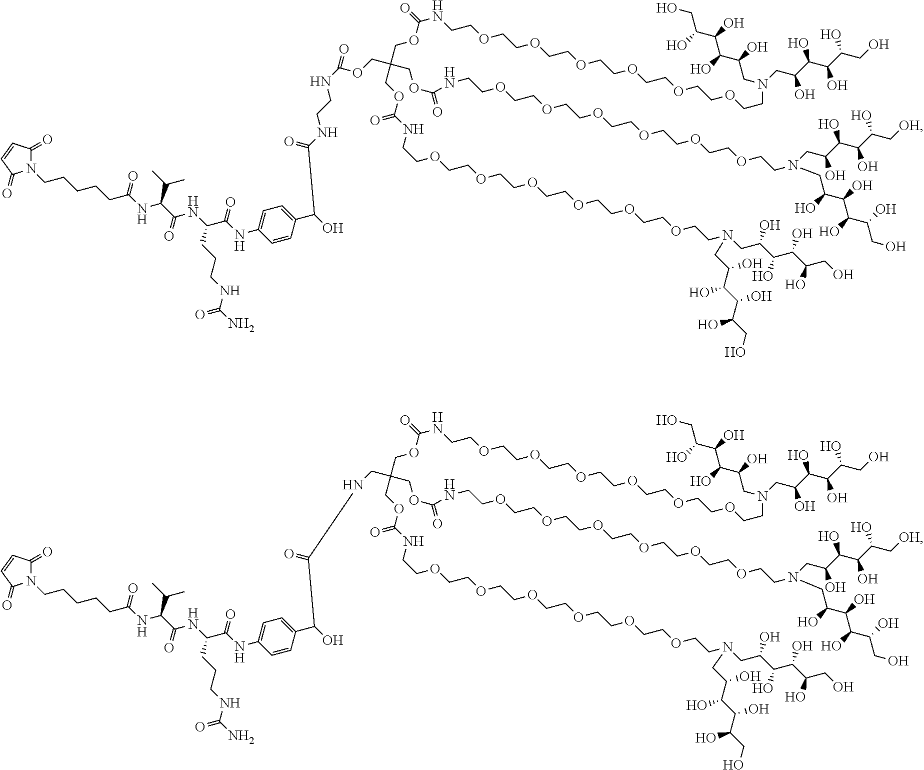 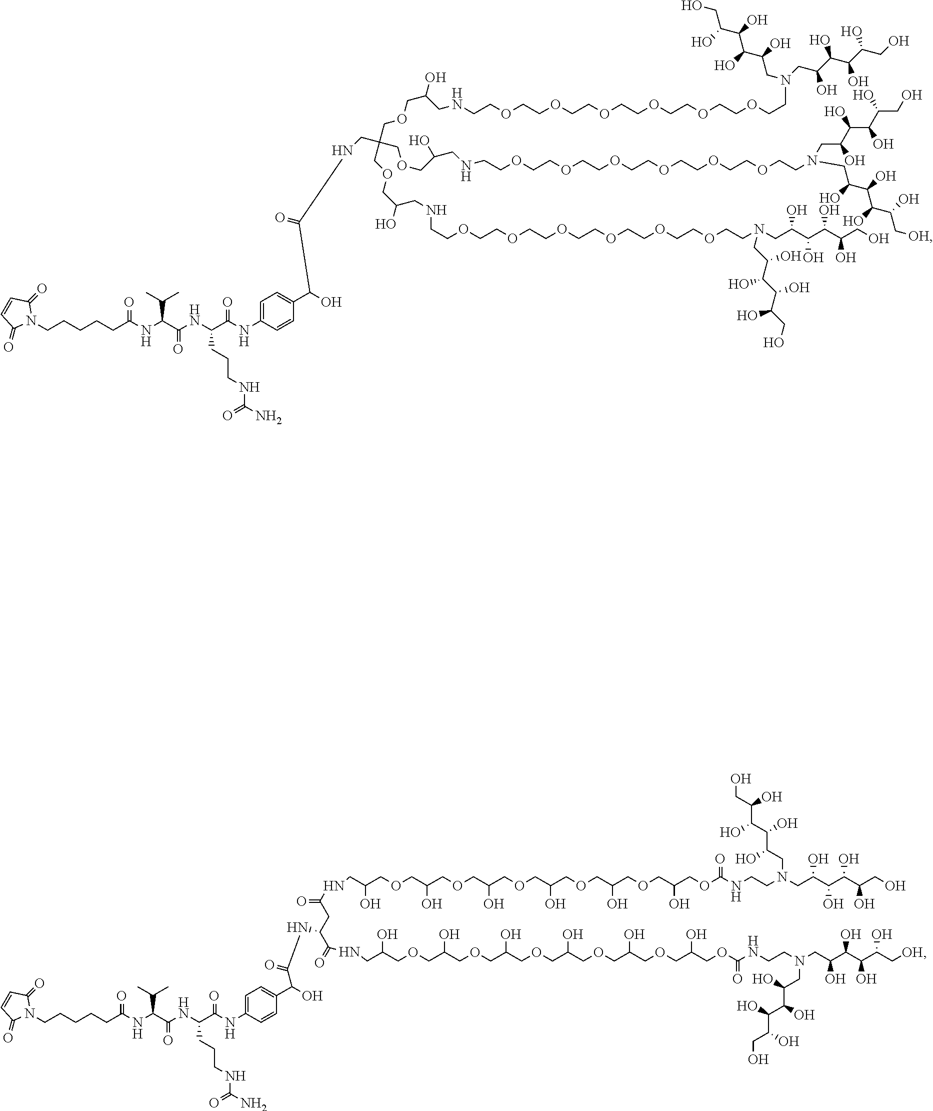 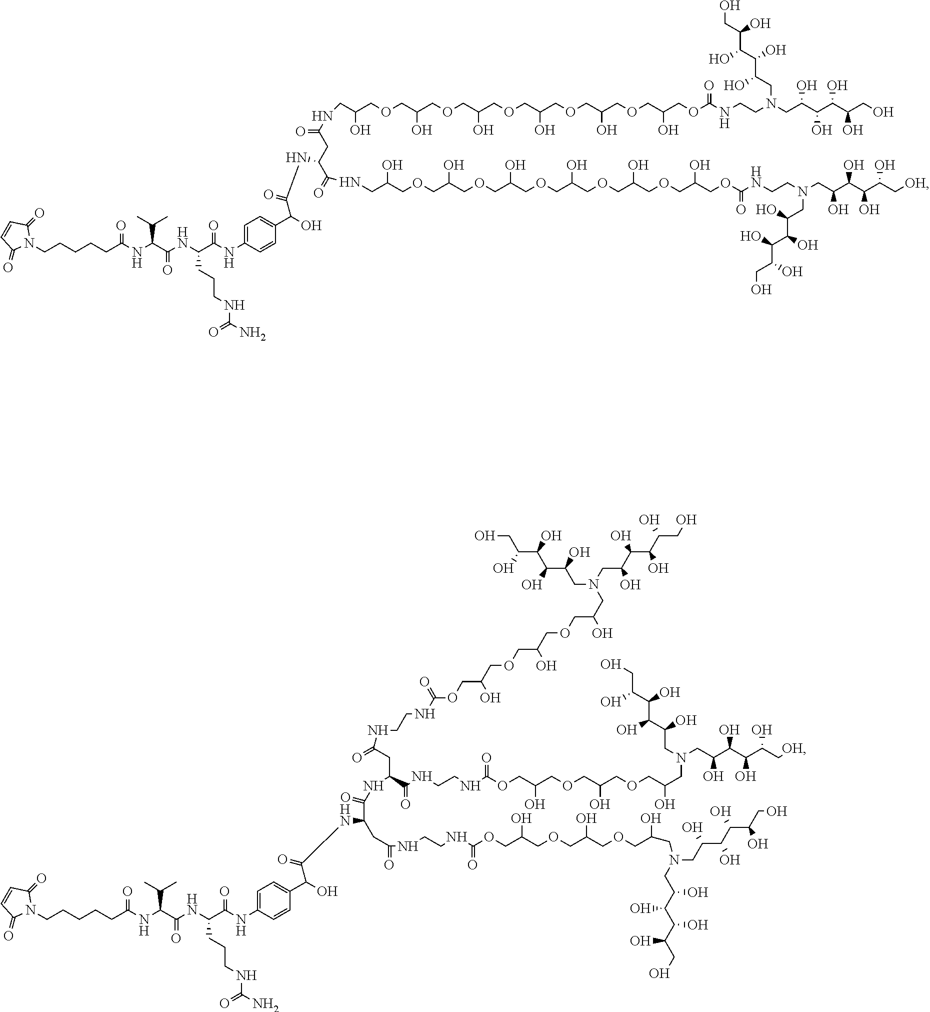 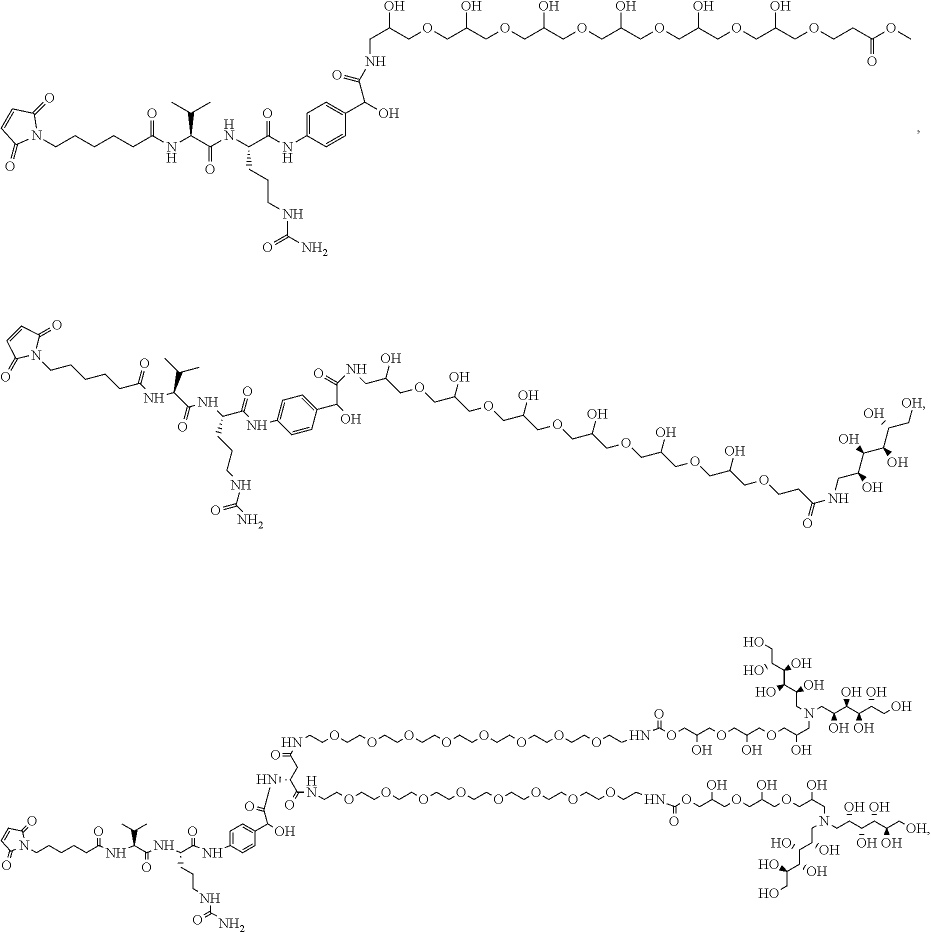 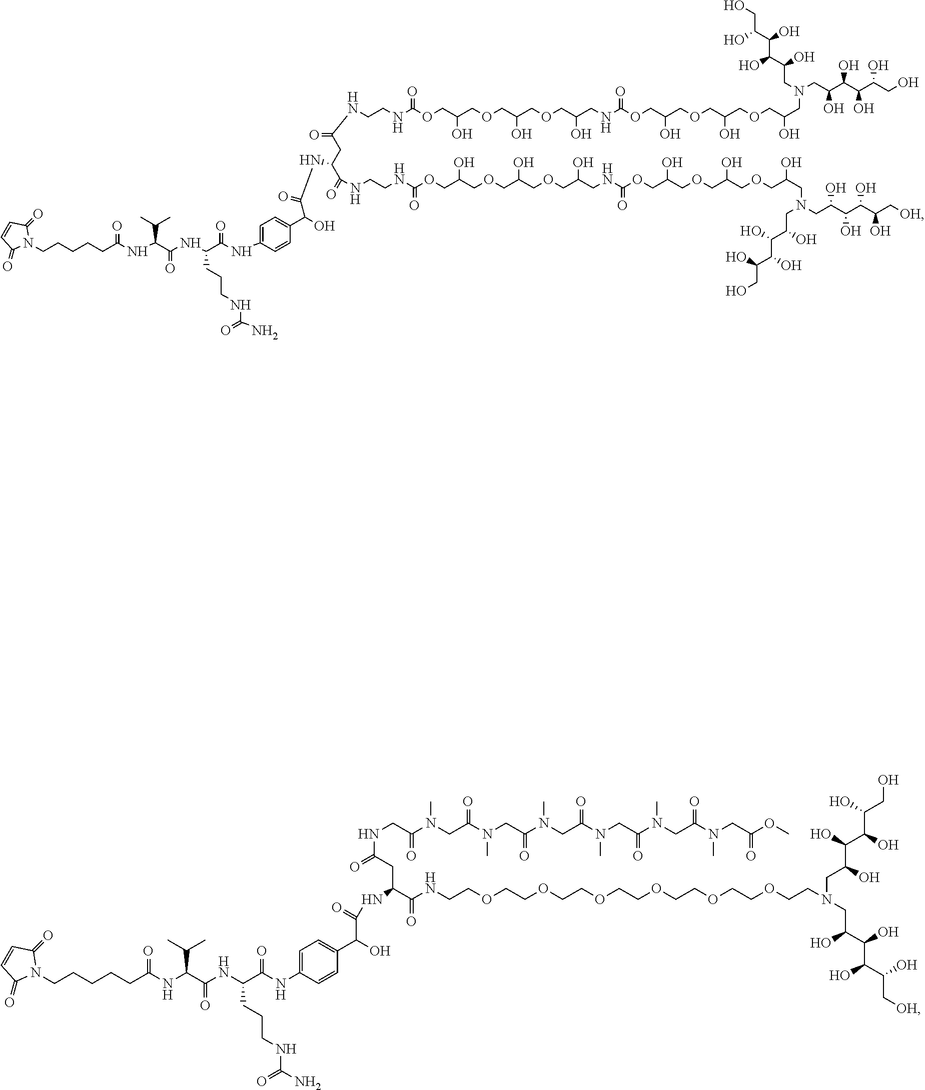 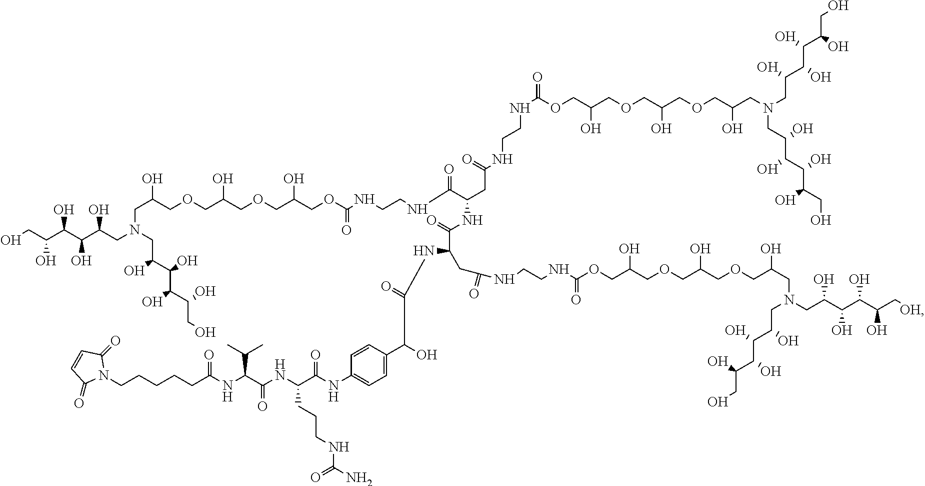 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 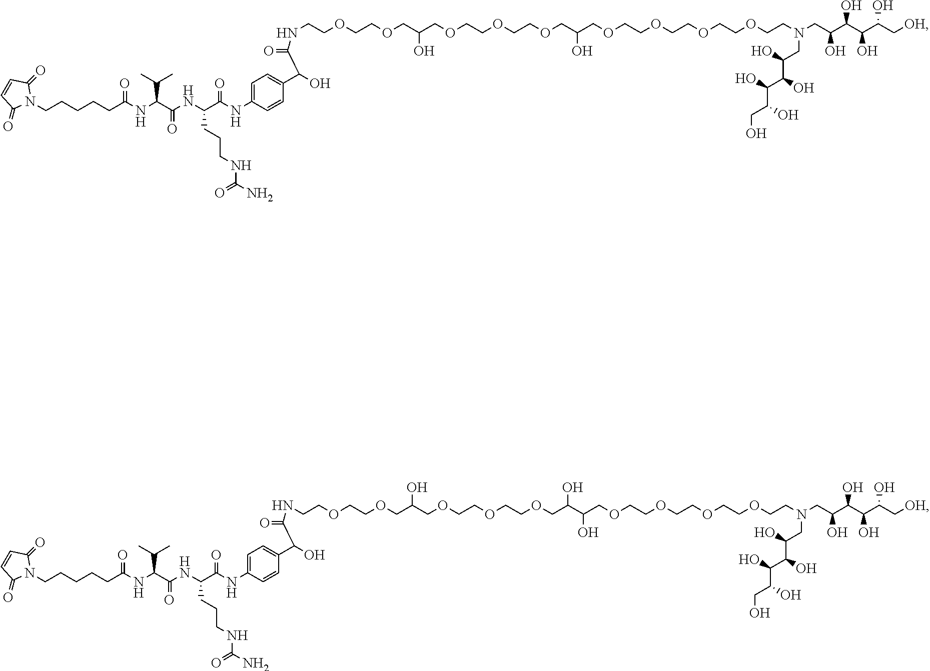 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)COCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)C(O)COCCOCCOCCOCCN(C[C@H](O)[C@@H](O)[C@H](O)[C@H](O)CO)C[C@H](O)[C@@H](O)[C@H](O)[C@H](O)CO)cc1] 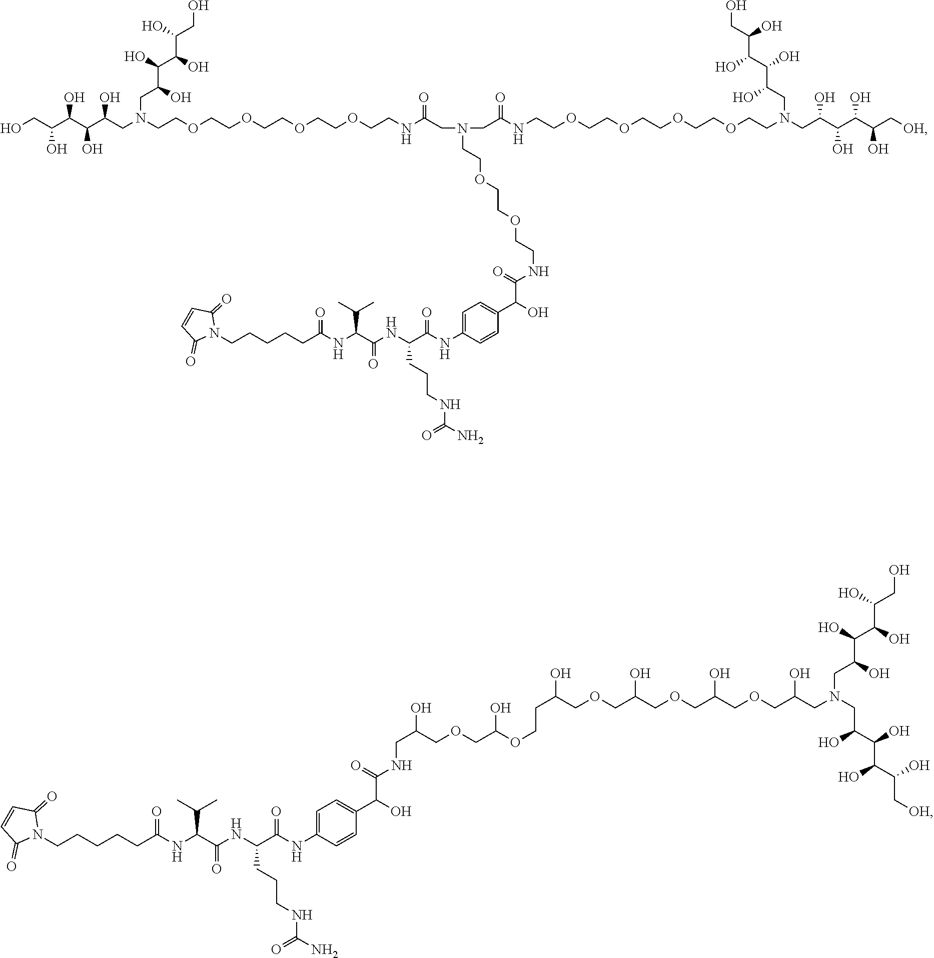 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)OCCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 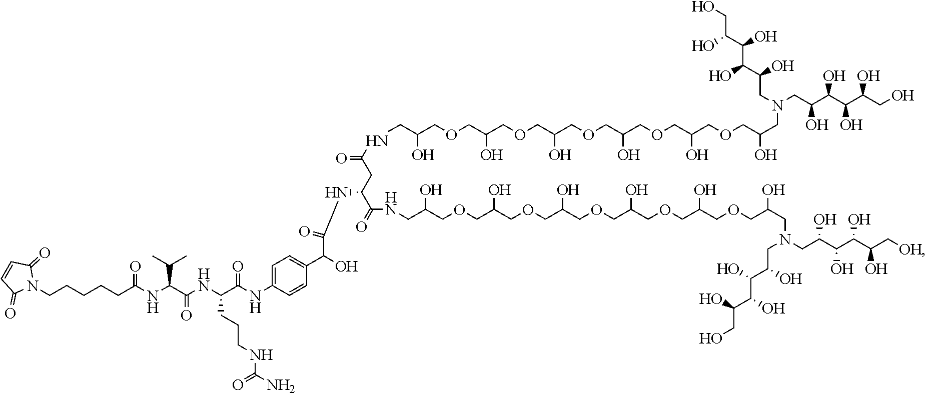 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1] 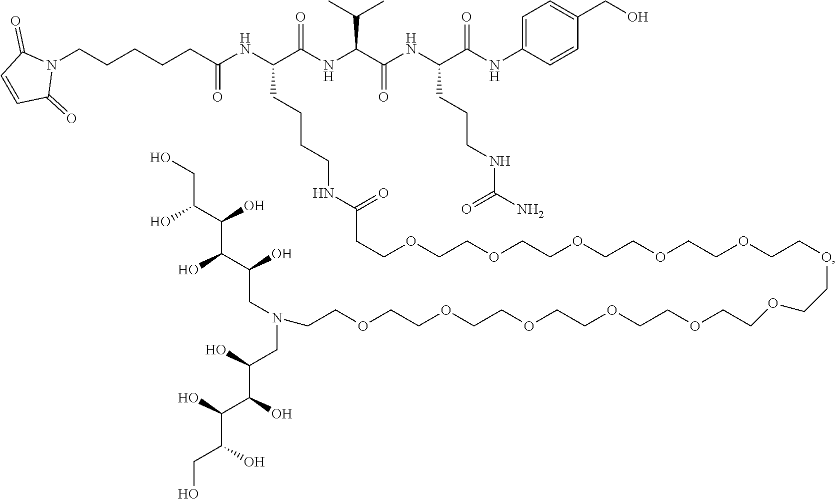 [SMILES: CC(C)[C@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](CCCNC(N)=O)C(=O)Nc1ccc(CO)cc1]
wherein the H on the benzylic OH is optionally replaced with a bond to one of the drug units or to the linking group attached to at least one of the drug units.

75. The conjugate of claim 30, wherein at least one of the drug units is attached to the linker compound, at the attachment site δ, to form a drug-linker compound, which can be attached to the binding agent to form the conjugate.

76. The conjugate of claim 75, wherein the drug unit is selected from a cytotoxic agent, an immune modulatory agent, a nucleic acid, a growth inhibitory agent, a PROTAC, a toxin, a radioactive isotope and a chelating ligand.

77. The conjugate of claim 76, wherein the drug unit is a cytotoxic agent.

78. The conjugate of claim 77, wherein the cytotoxic agent is selected from the group consisting of an auristatin, a maytansinoid, a camptothecin, a duocarmycin, and a calicheamicin.

79. The conjugate of claim 78, wherein the cytotoxic agent is MMAE, MMAF, exatecan, RS-exatecan, SS-exatecan, SN-38, D×D, maytansine, maytansinol or ansamatocin-2.

80. (canceled)

81. The conjugate of claim 78, wherein the cytotoxic agent is exatecan.

82. The conjugate of claim 76, wherein the drug unit is an immune modulatory agent.

83. The conjugate of claim 82, wherein the immune modulatory agent is selected from a TRL7 agonist, a TLR8 agonist, a STING agonist, or a RIG-I agonist.

84. (canceled)

85. The conjugate of claim 83, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

86. (canceled)

87. The conjugate of claim 83, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

88.-89. (canceled)

90. The conjugate of claim 83, wherein the RIG-I agonist is selected from KIN1148, SB-9200, KIN700, KIN600, KIN500, KIN100, KIN101, KIN400 and KIN2000.

91. The conjugate of claim 76, wherein the drug unit is a chelating ligand.

92. The conjugate of claim 91, wherein the chelating ligand is selected from platinum (Pt), ruthenium (Ru), rhodium (Rh), gold (Au), silver (Ag), copper (Cu), molybdenum (Mo), titanium (Ti), or iridium (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

93. The conjugate of claim 75, wherein the drug-linker has one of the following structures:

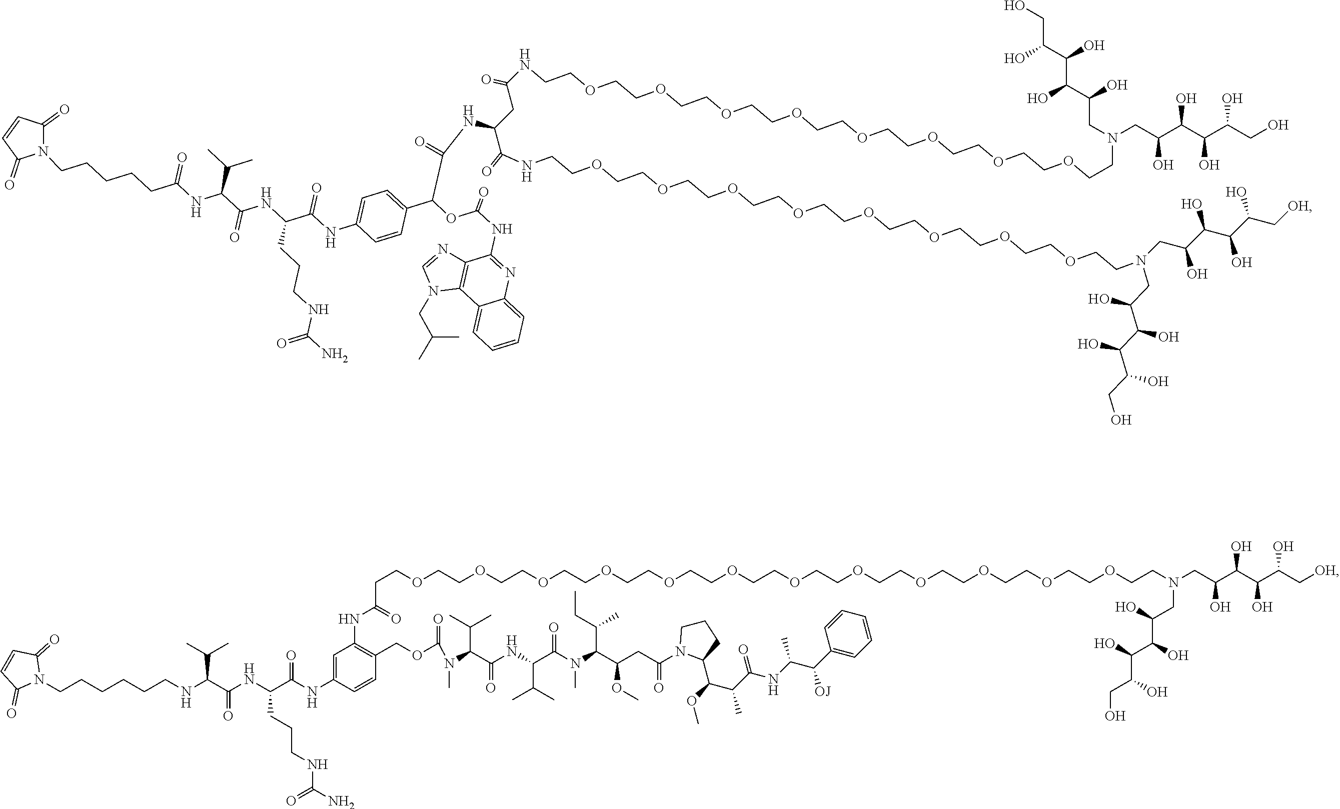

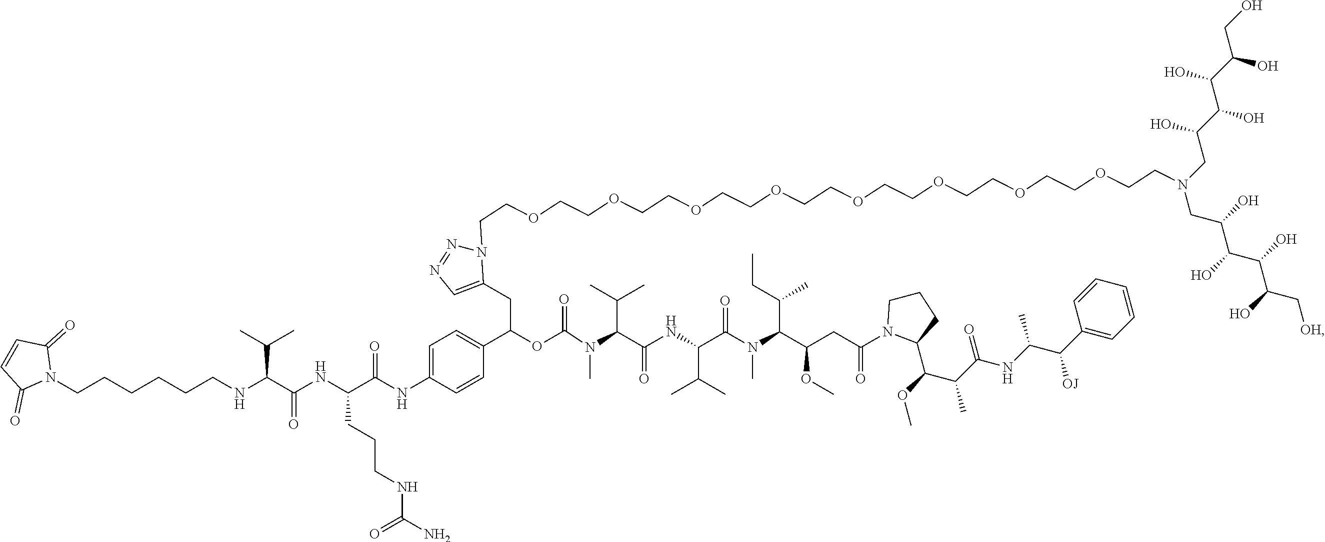

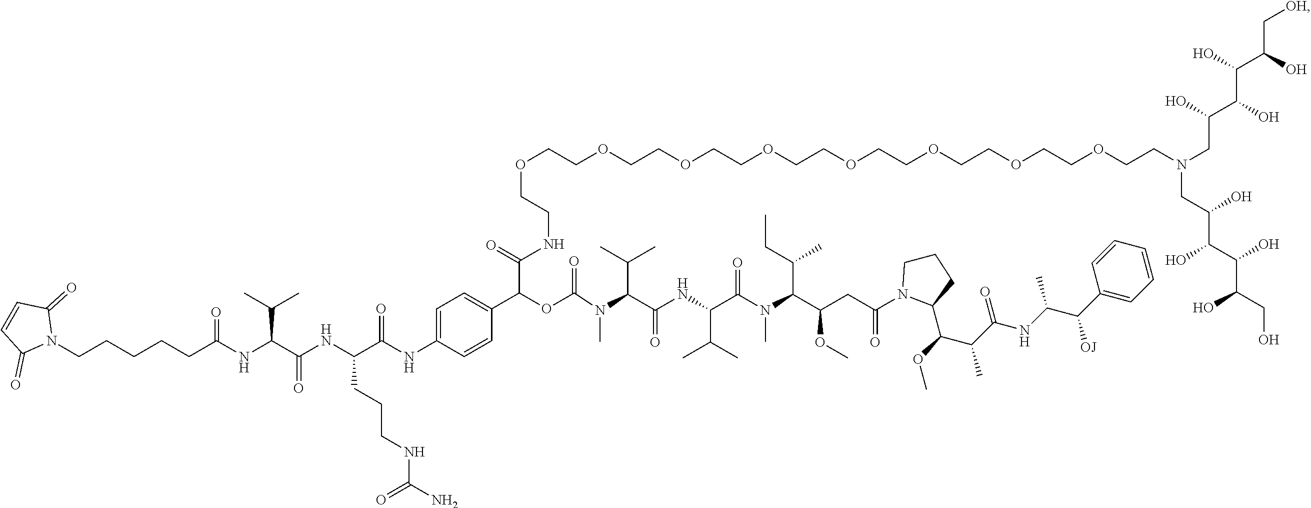

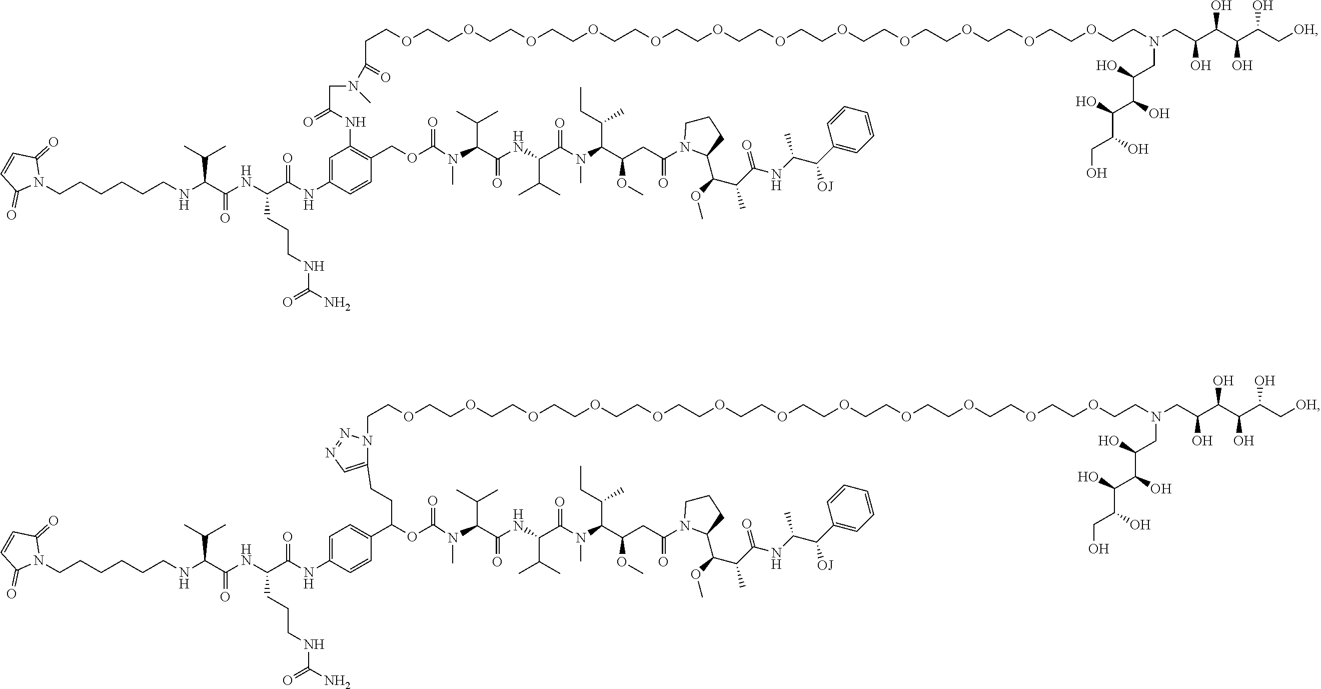

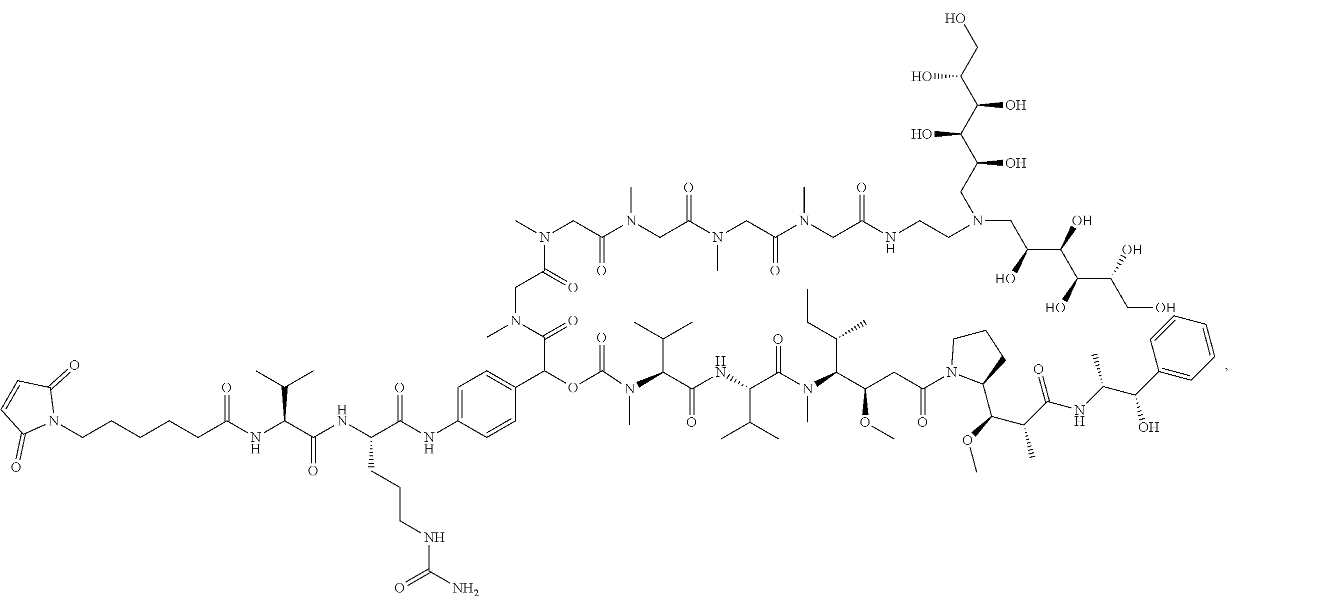 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

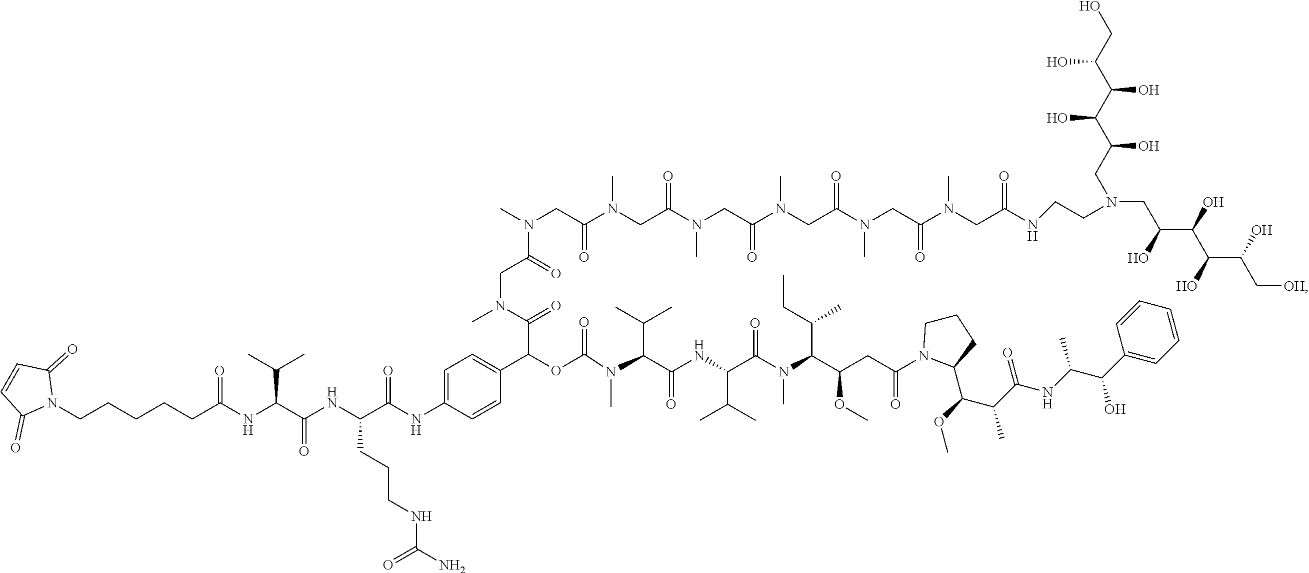 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

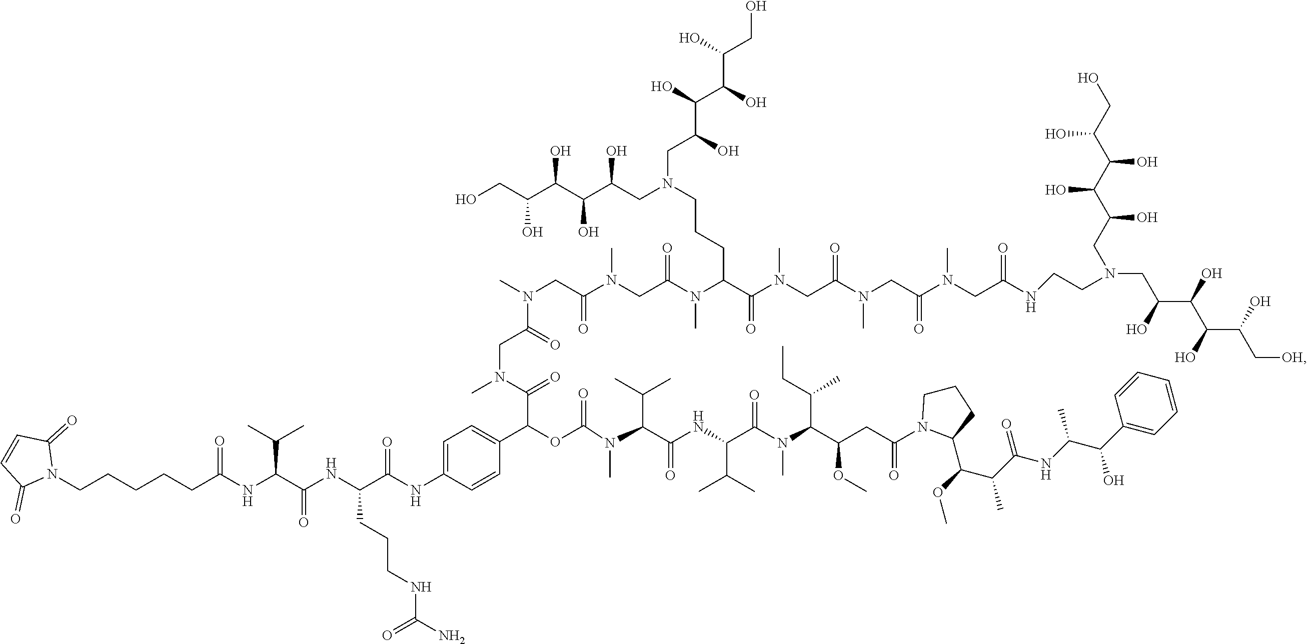 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

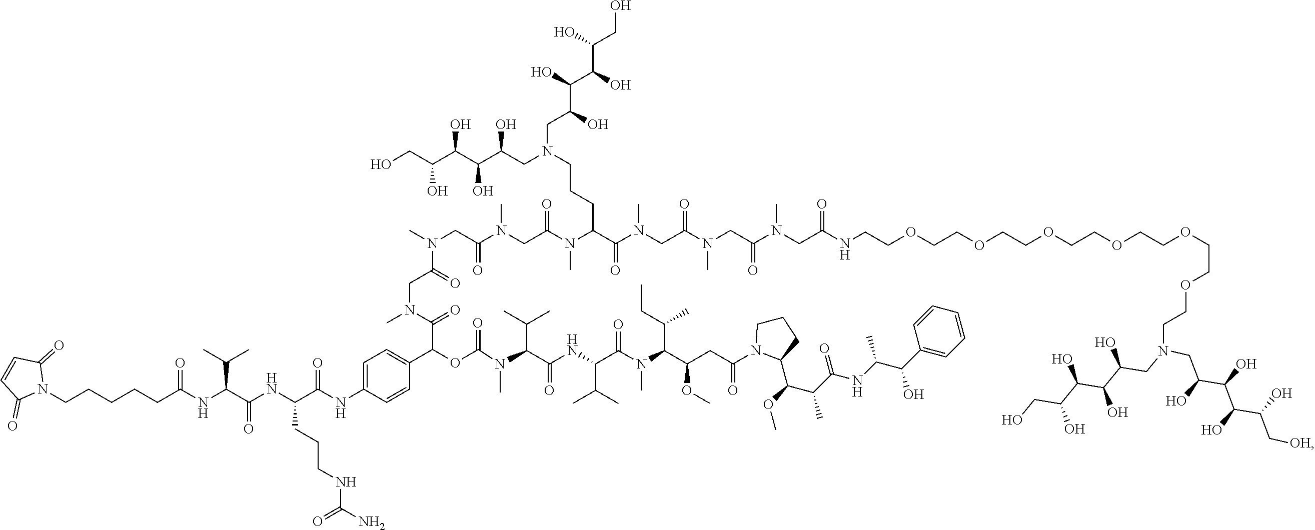 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

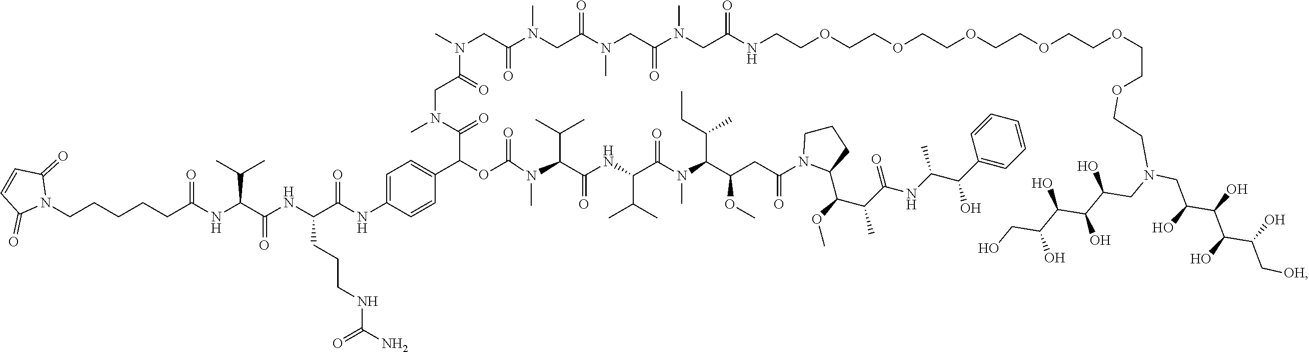 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

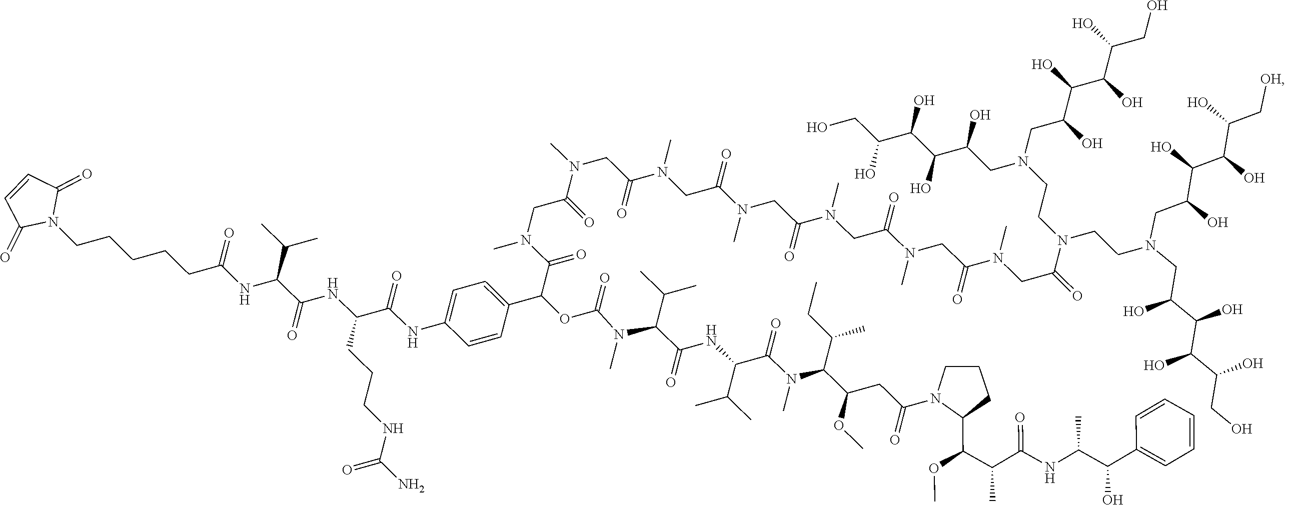 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

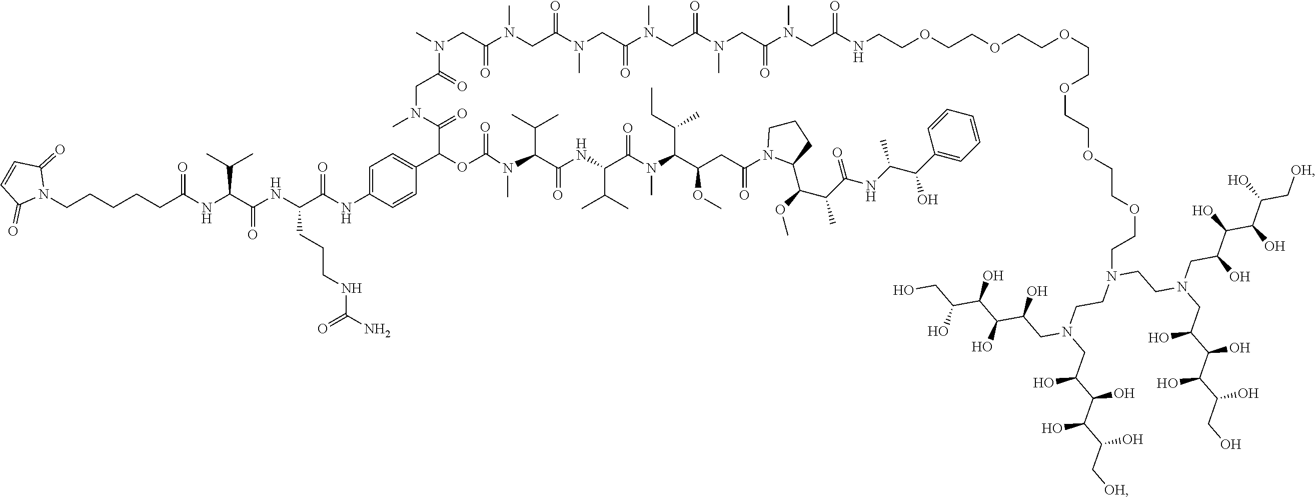 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

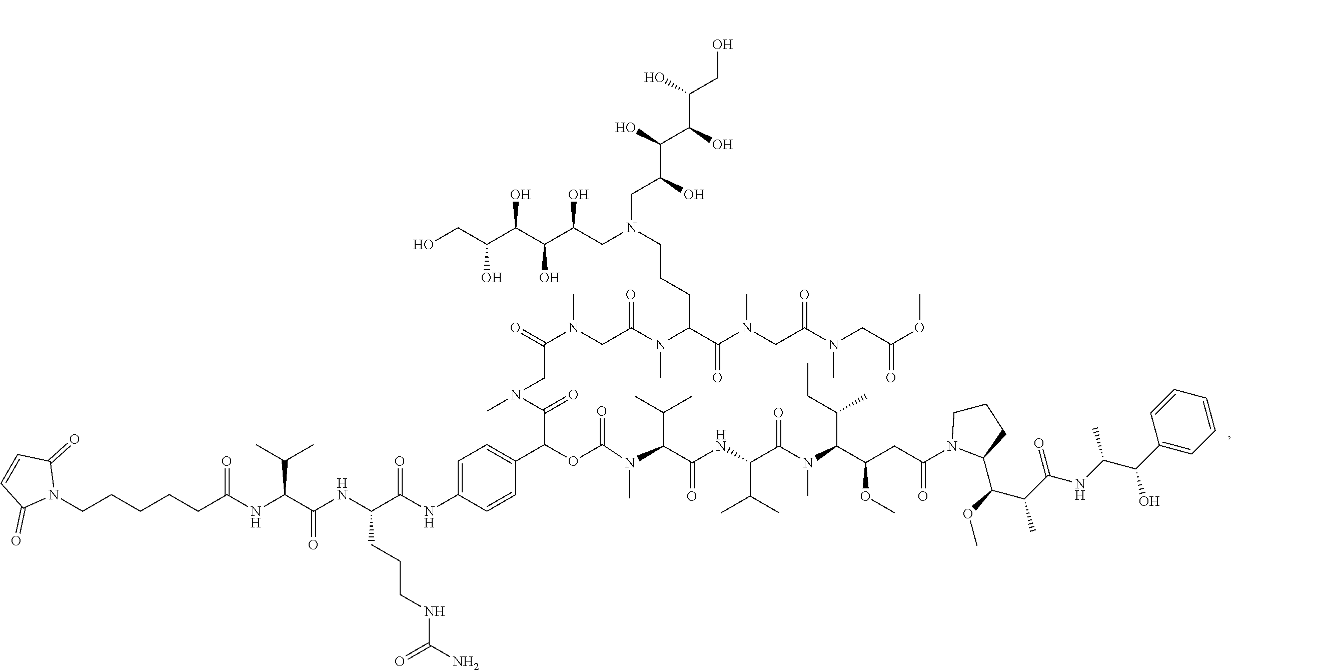 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

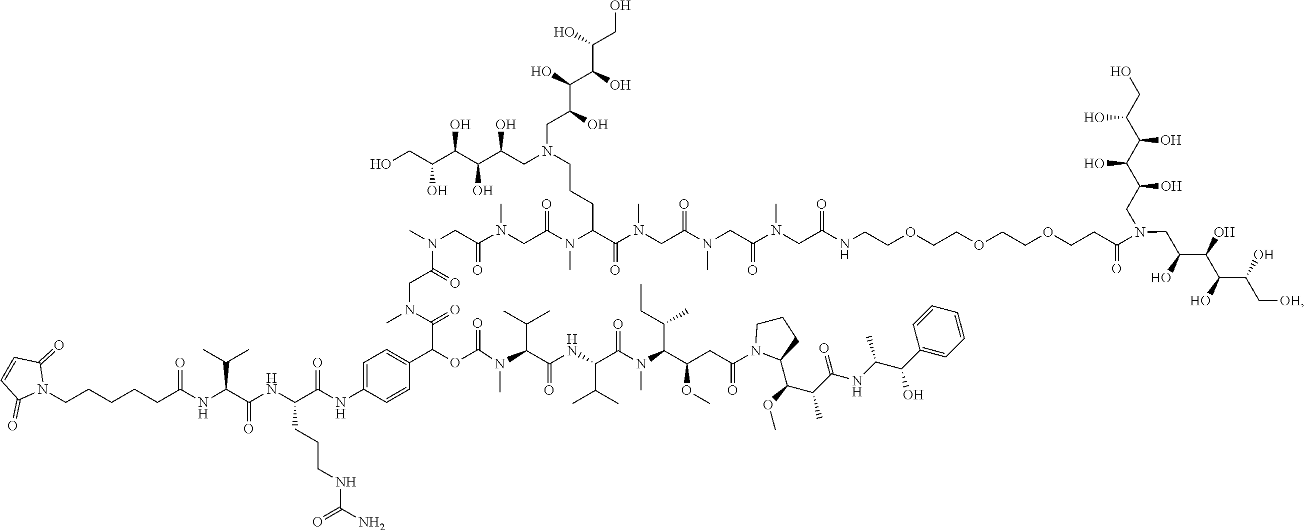 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

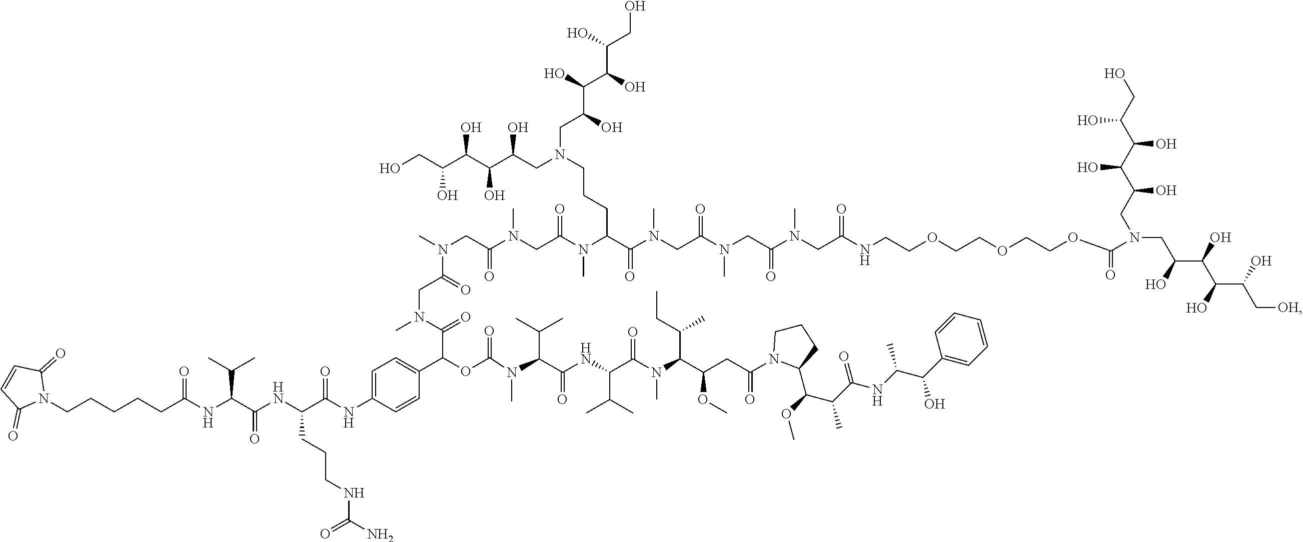 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

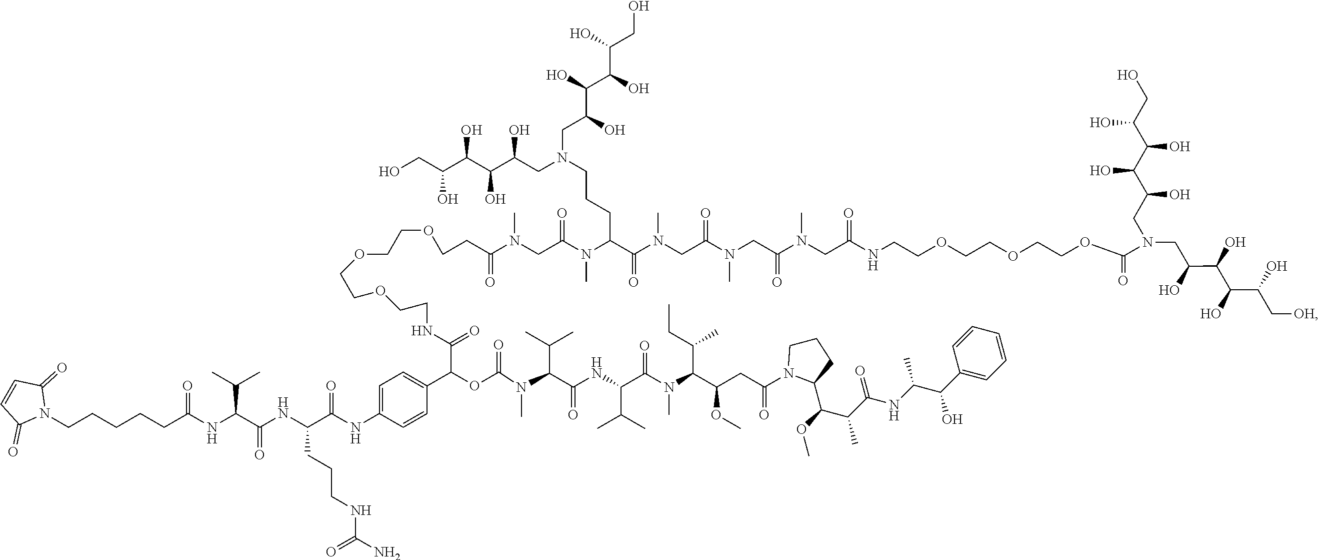 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

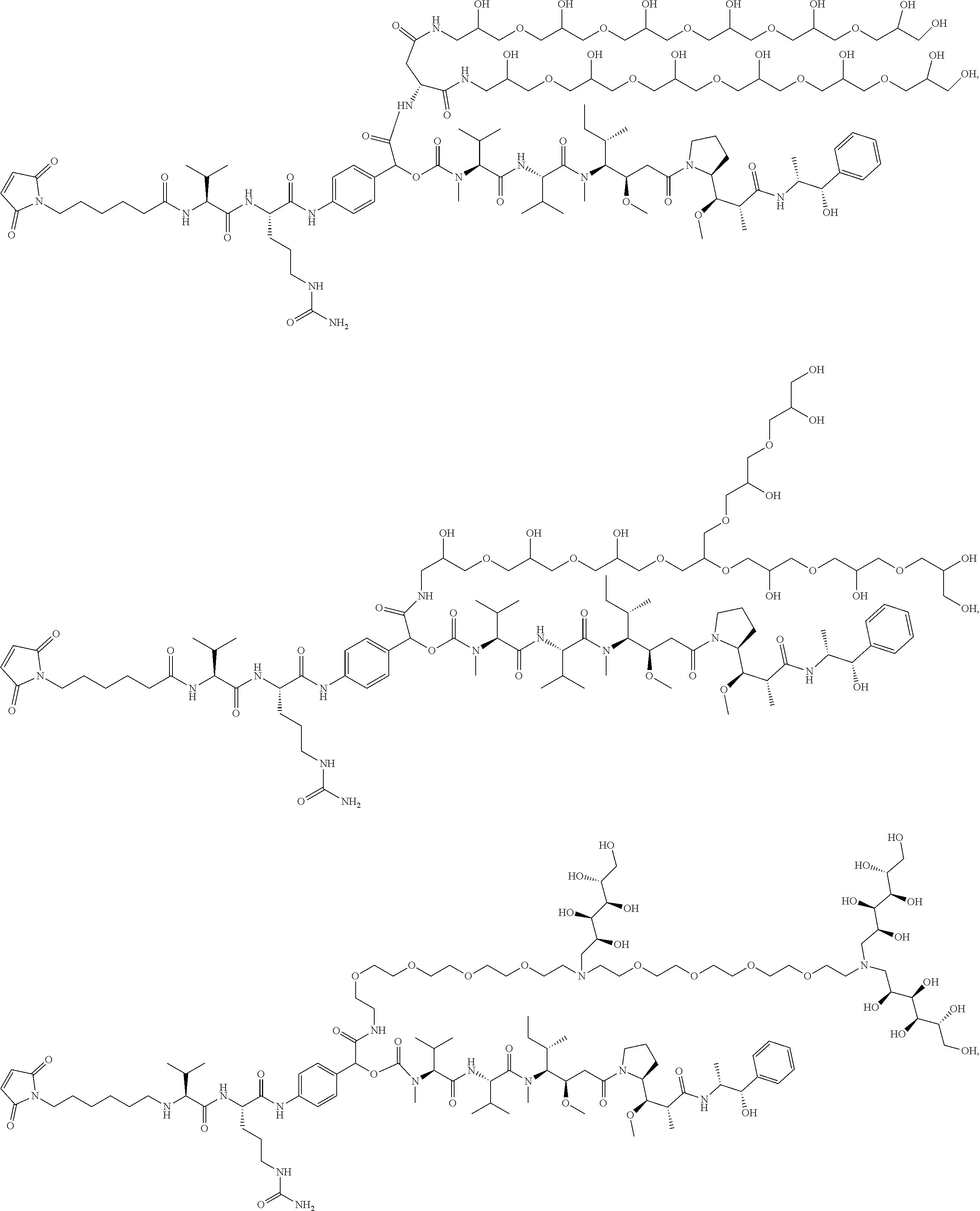

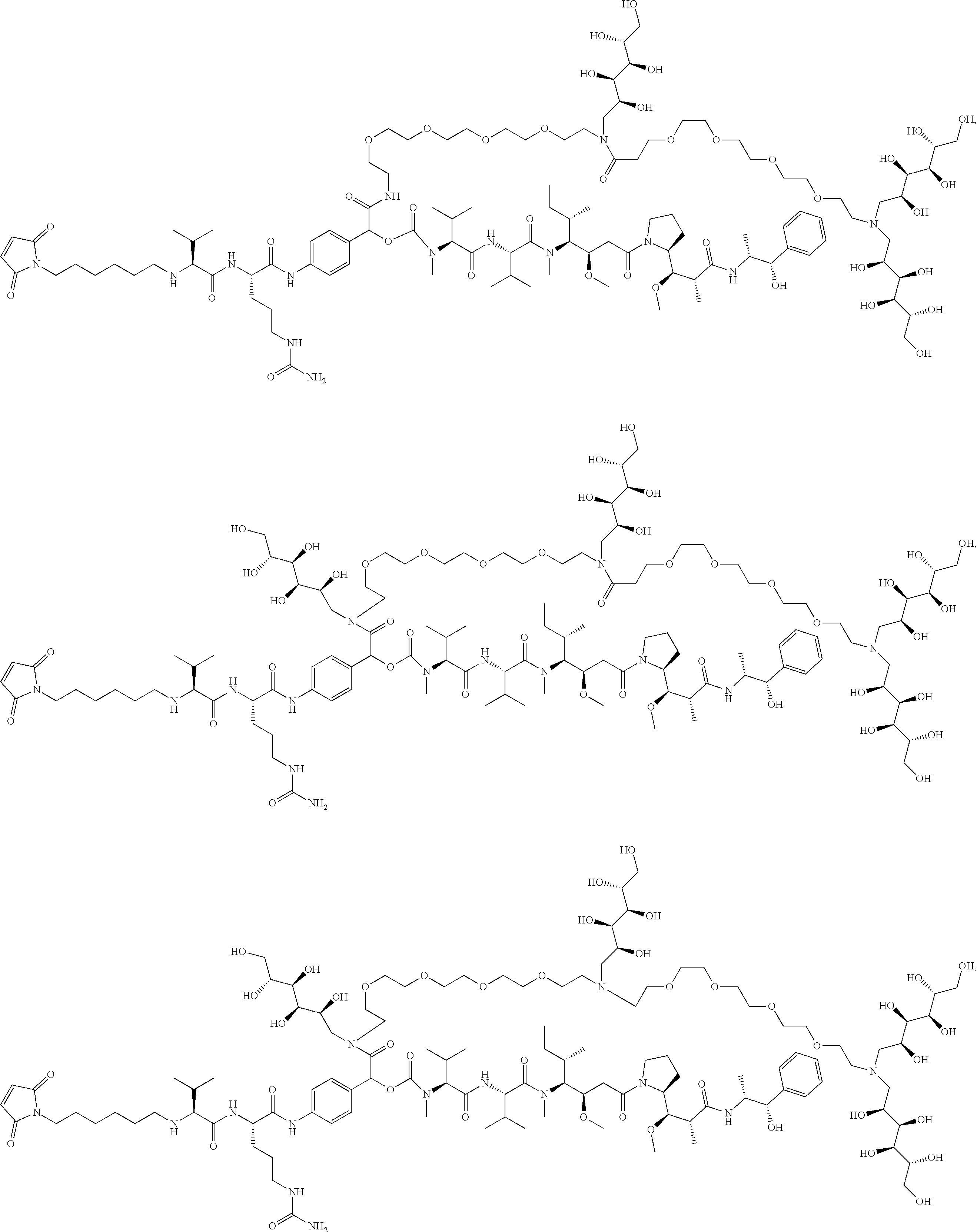

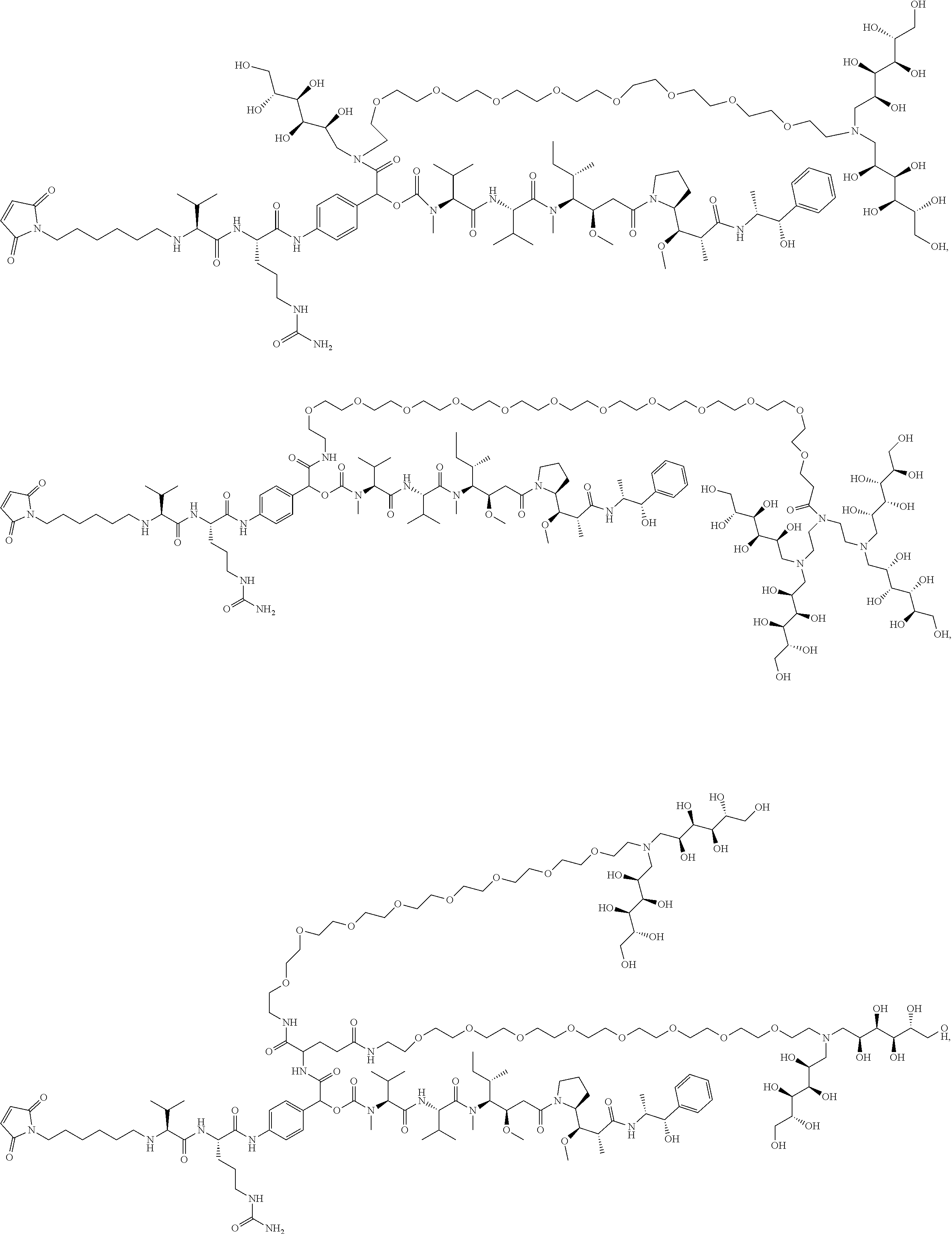

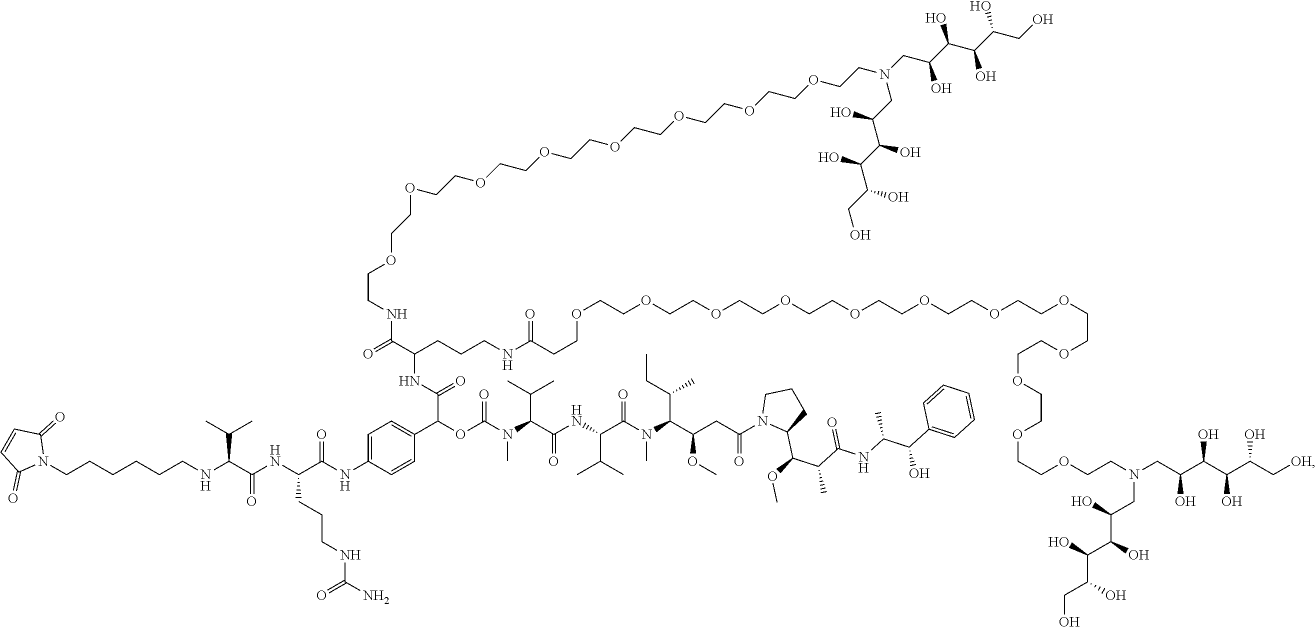 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

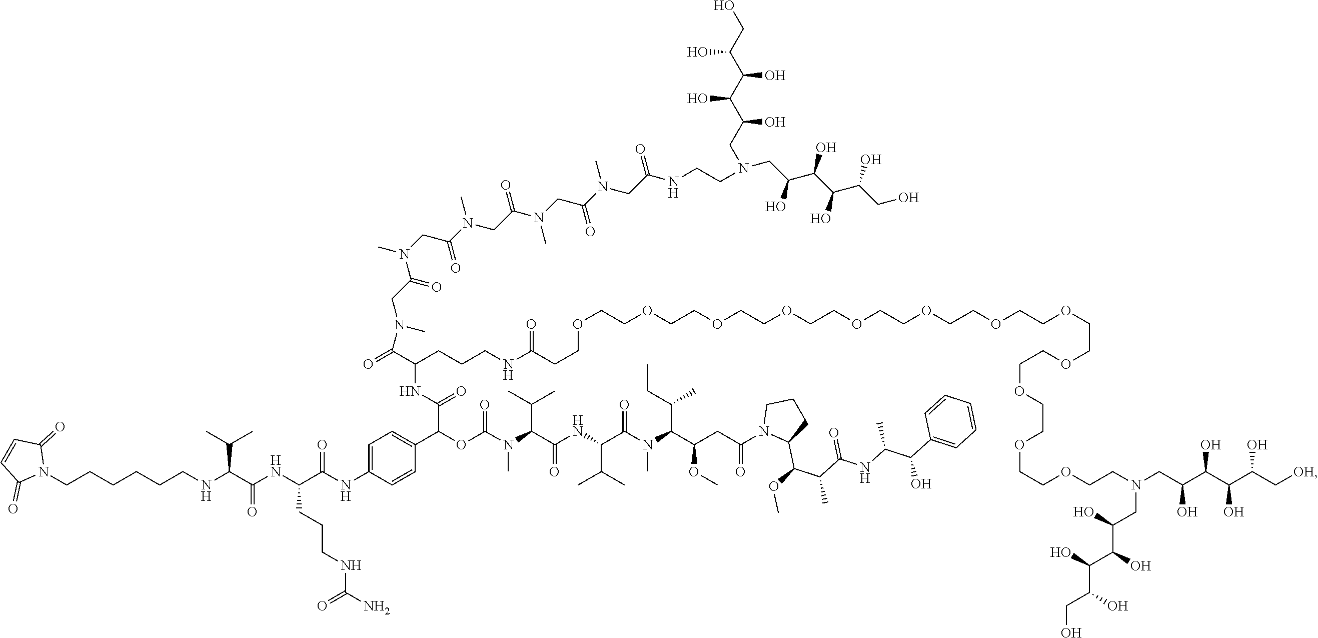 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

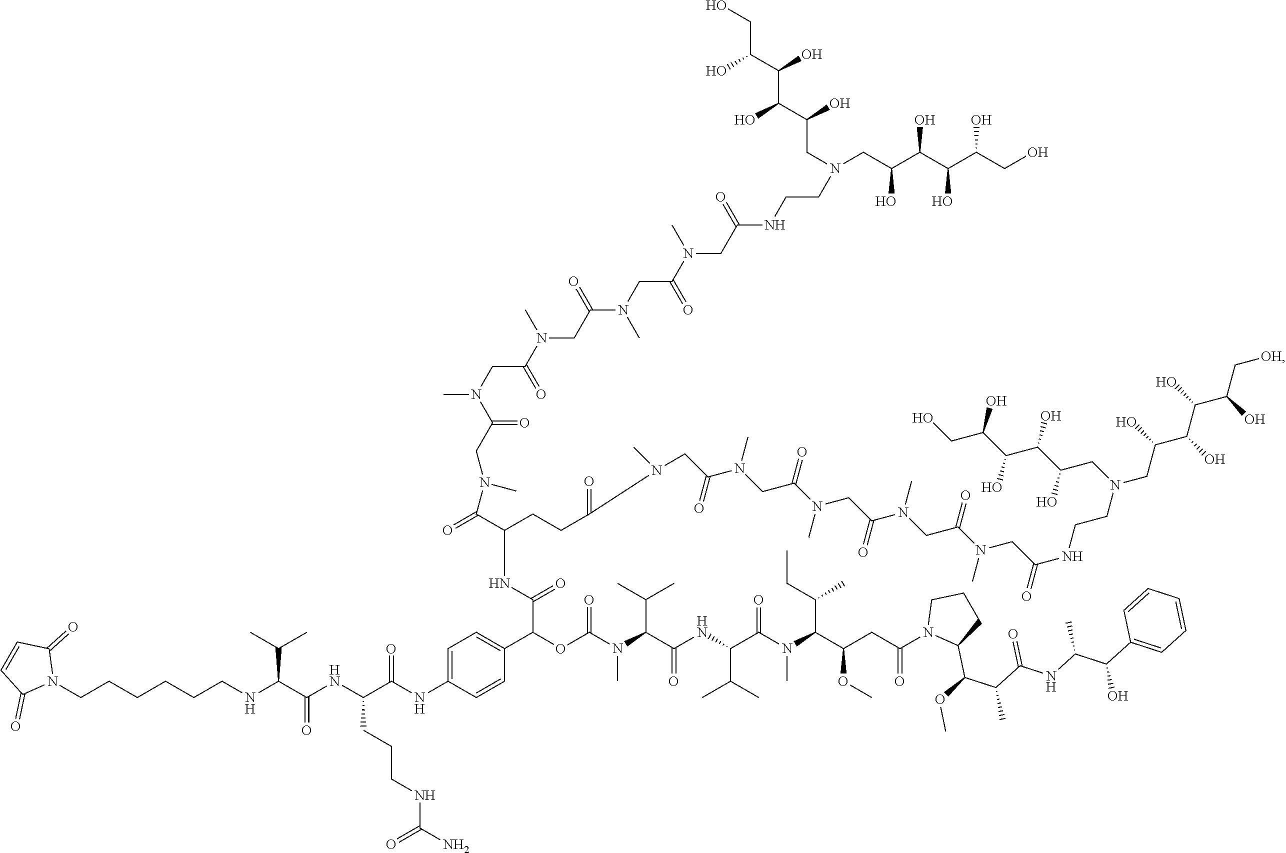 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

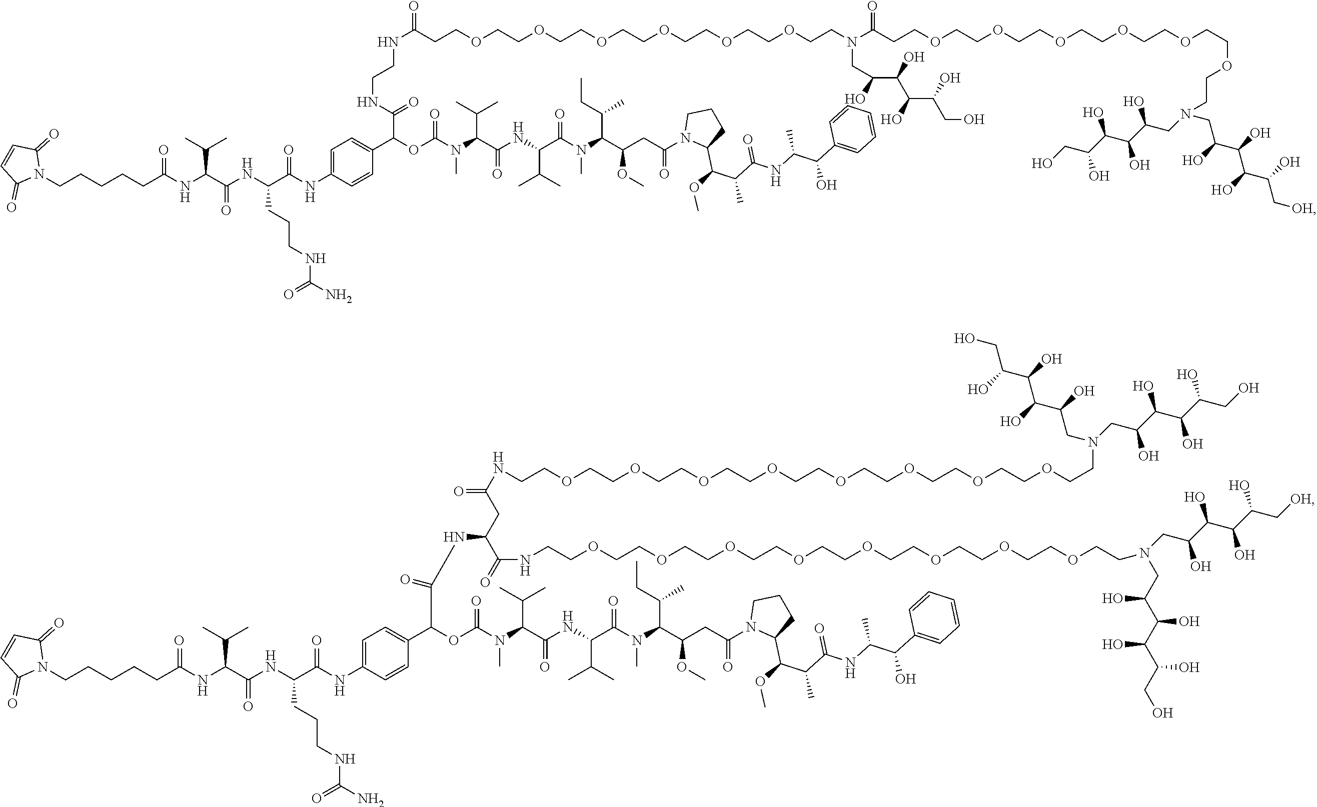

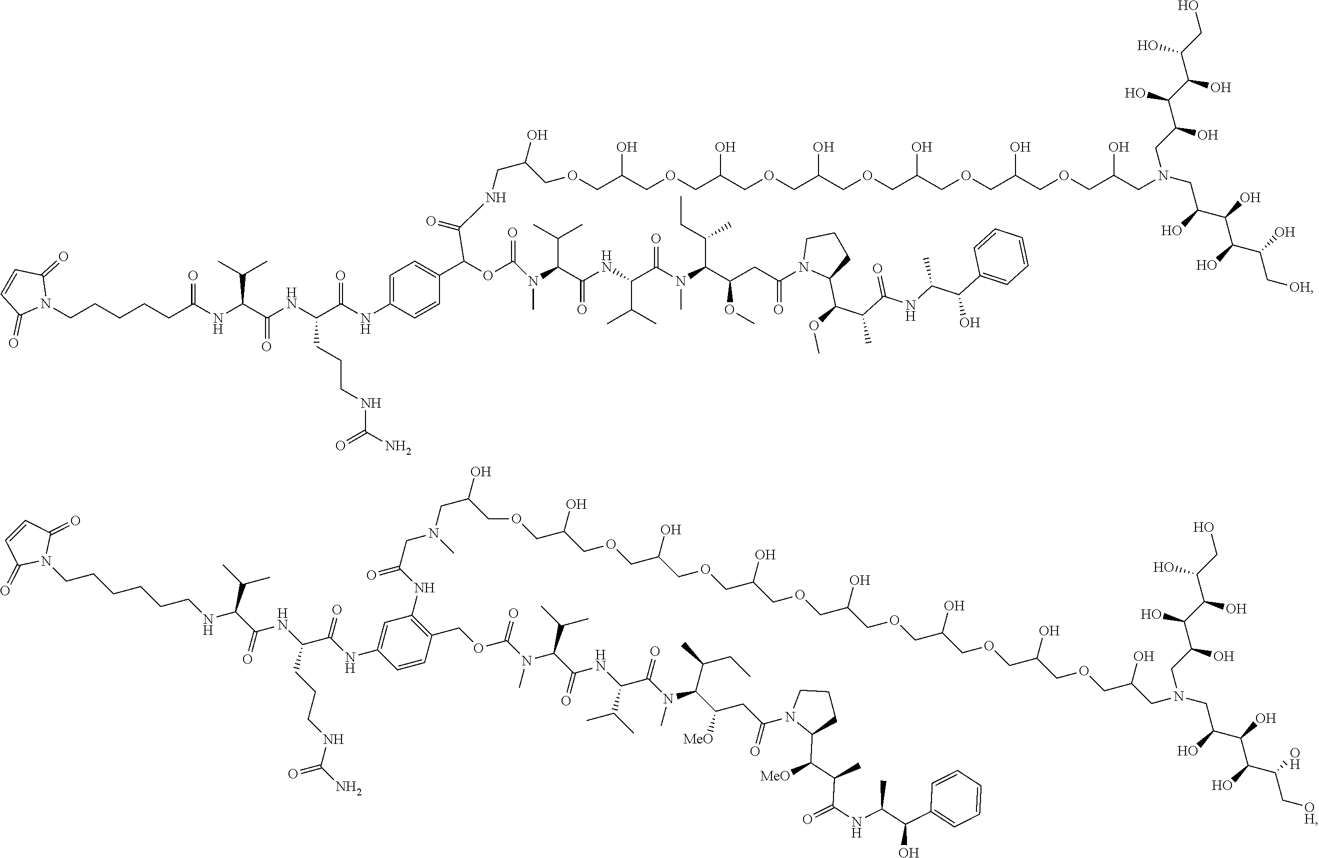

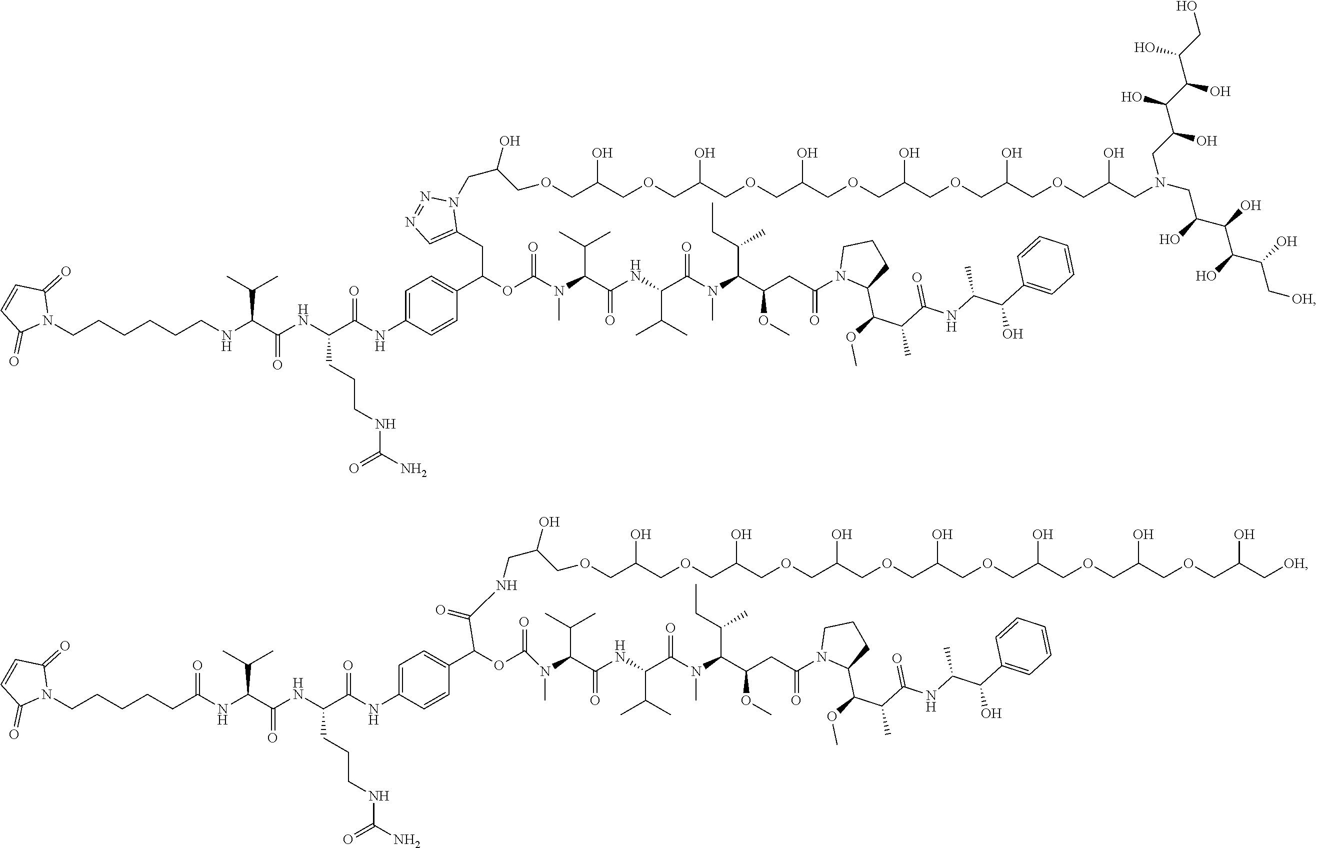

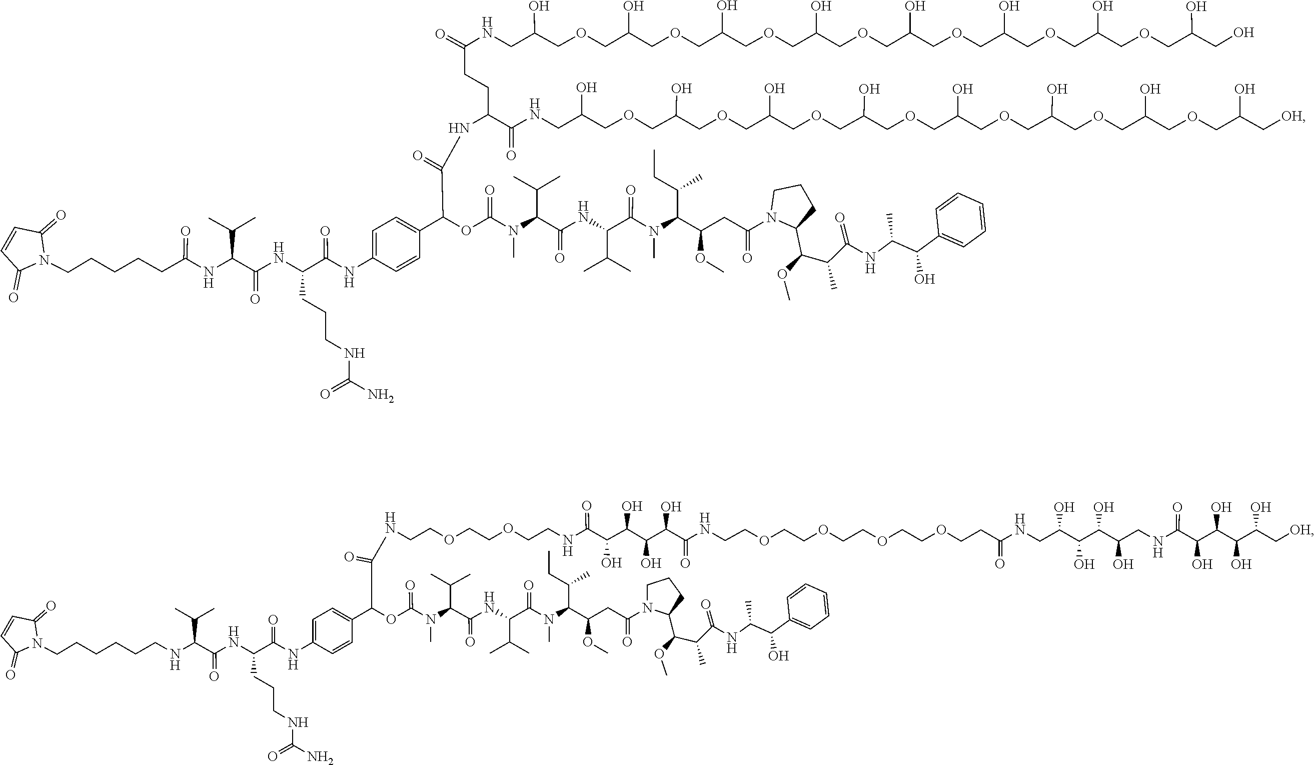

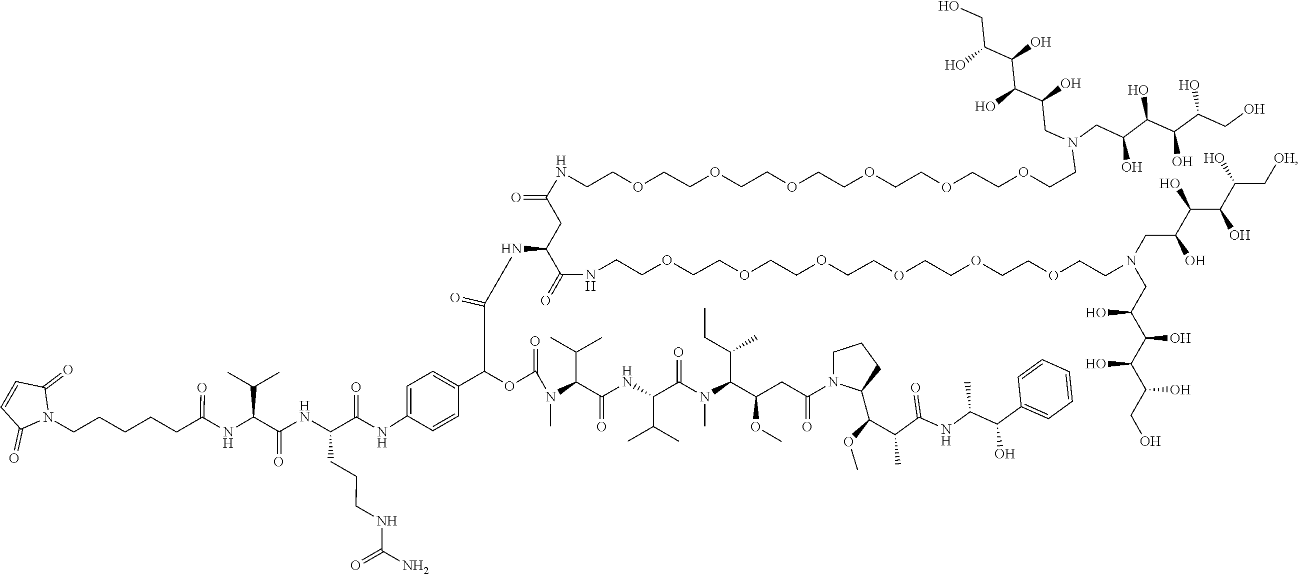 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

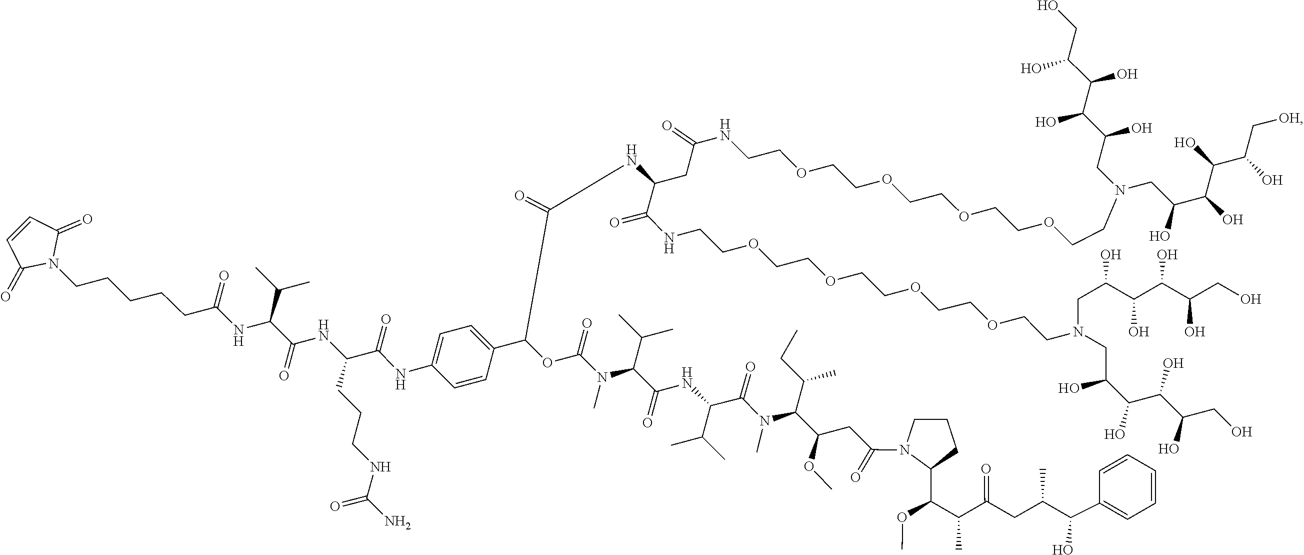 [SMILES: CC[C@H](C)[C@@H]([C@@H](CC(=O)N1CCC[C@H]1[C@H](OC)[C@@H](C)C(=O)C[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

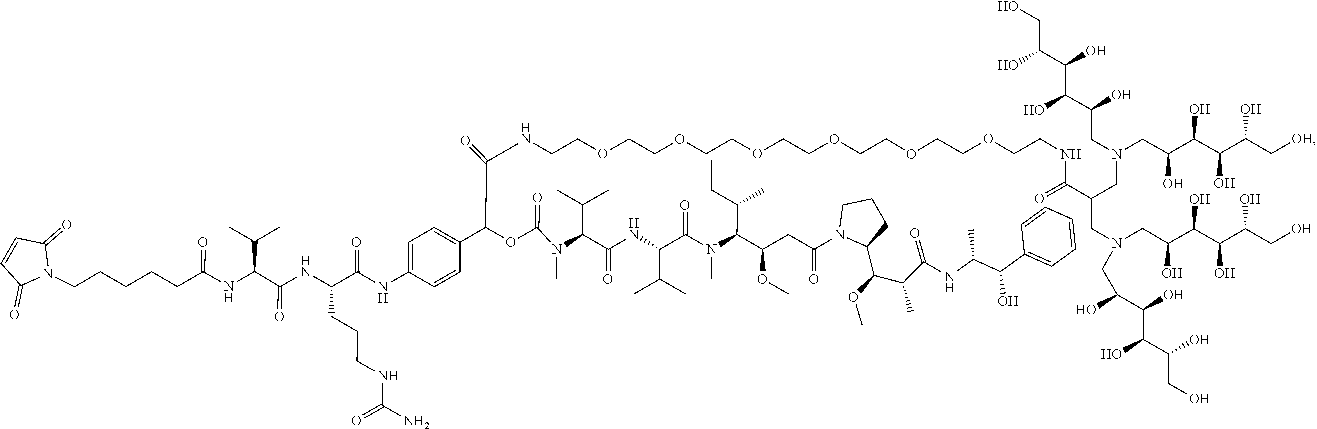 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

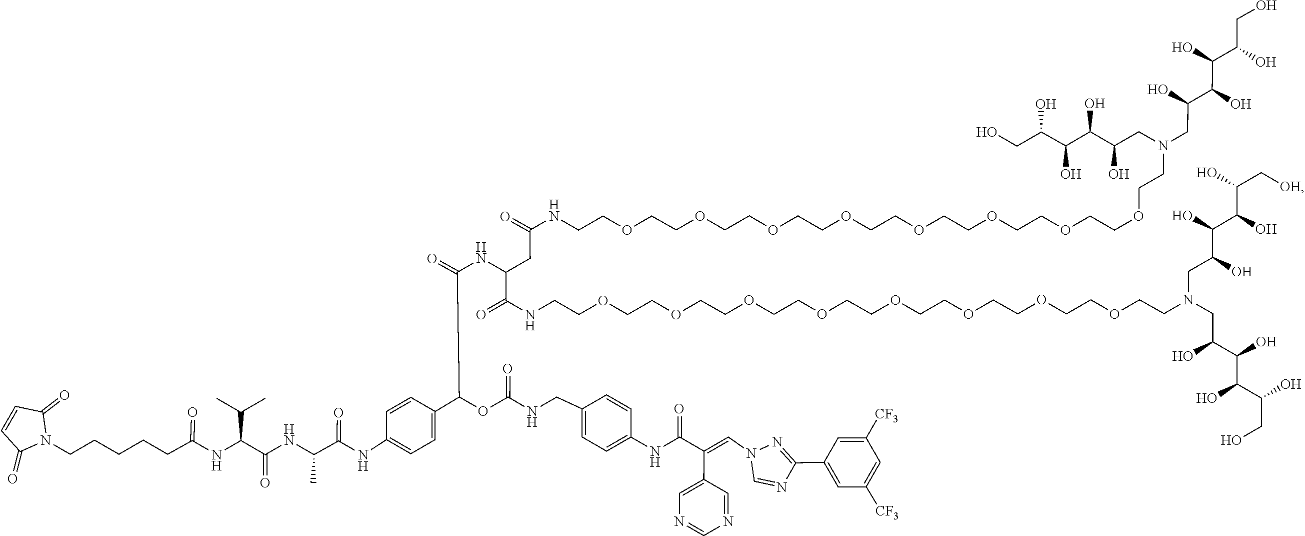 [SMILES: CC(C)[C@H](NC(=O)CCCCCN1C(=O)C=CC1=O)C(=O)N[C@@H](C)C(=O)Nc1ccc(C(OC(=O)NCc2ccc(NC(=O)/C(=C/n3cnc(-c4cc(C(F)(F)F)cc(C(F)(F)F)c4)n3)c3cncnc3)cc2)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

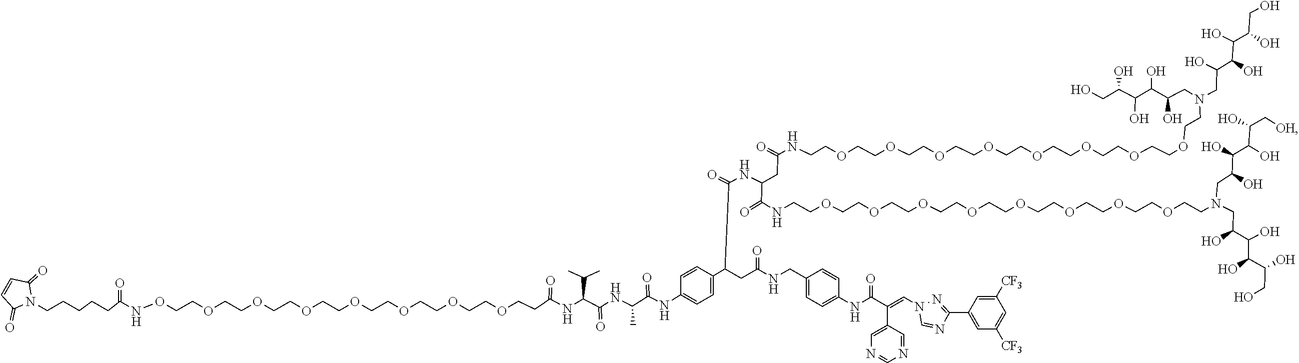 [SMILES: CC(C)[C@H](NC(=O)CCOCCOCCOCCOCCOCCOCCOCCONC(=O)CCCCCN1C(=O)C=CC1=O)C(=O)N[C@@H](C)C(=O)Nc1ccc(C(CC(=O)NCc2ccc(NC(=O)/C(=C/n3cnc(-c4cc(C(F)(F)F)cc(C(F)(F)F)c4)n3)c3cncnc3)cc2)C(=O)NC(CC(=O)NCCOCCOCCOCCOCCOCCOCCOCCOCCN(CC(O)[C@H](O)C(O)[C@@H](O)CO)C[C@@H](O)C(O)C(O)[C@@H](O)CO)C(=O)NCCOCCOCCOCCOCCOCCOCCOCCOCCN(C[C@H](O)C(O)[C@H](O)[C@H](O)CO)C[C@H](O)[C@@H](O)C(O)[C@H](O)CO)cc1]

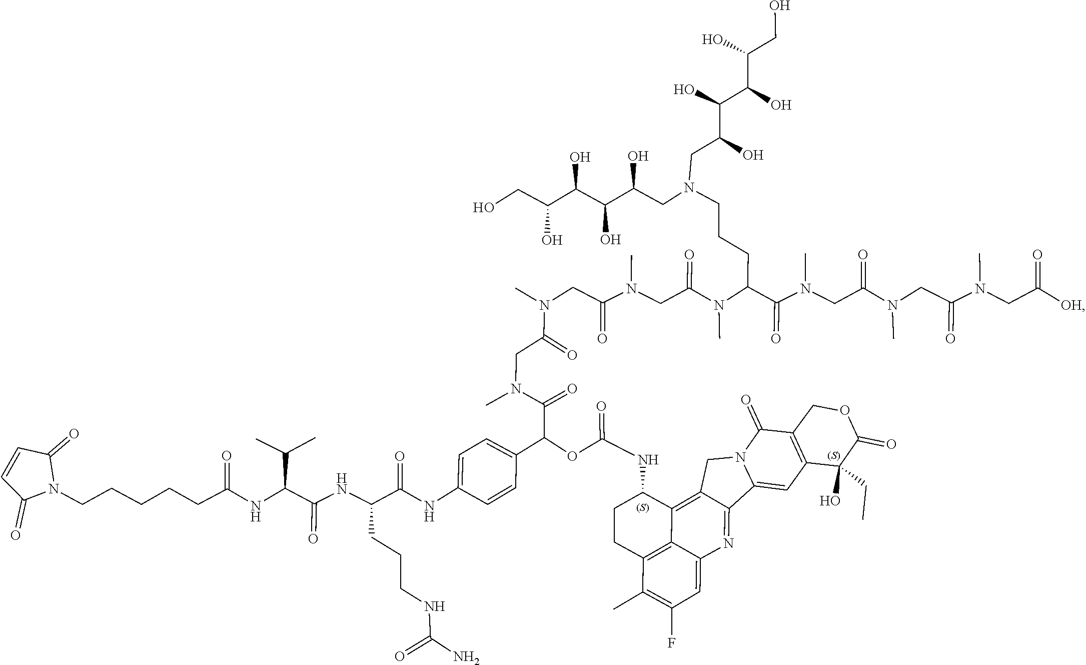

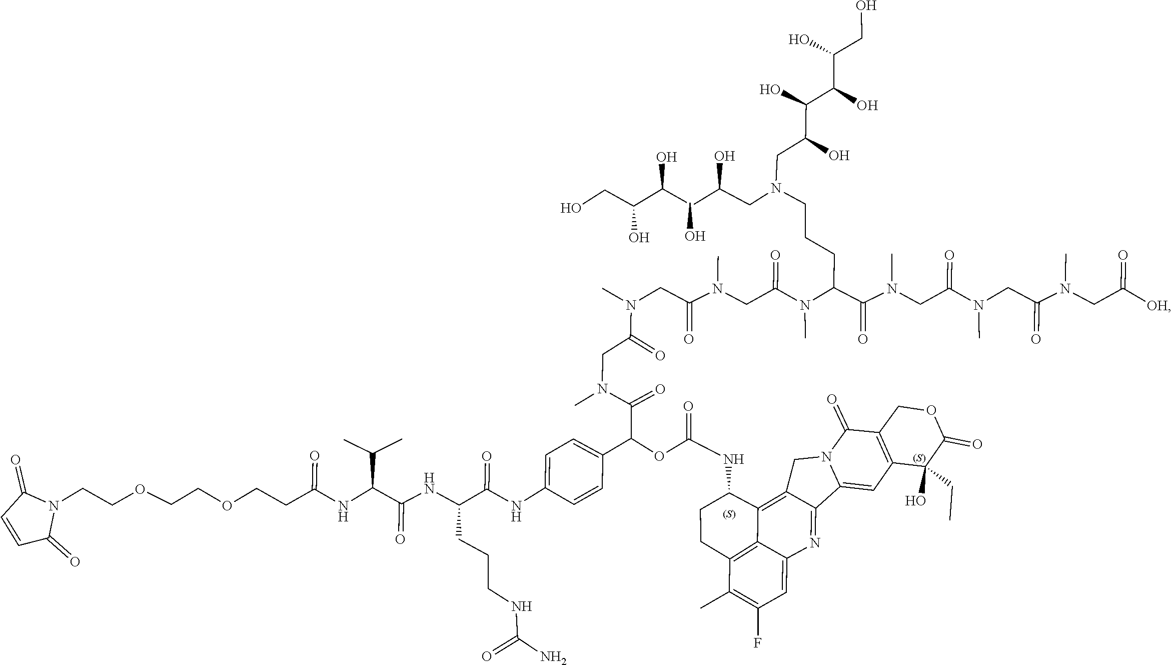

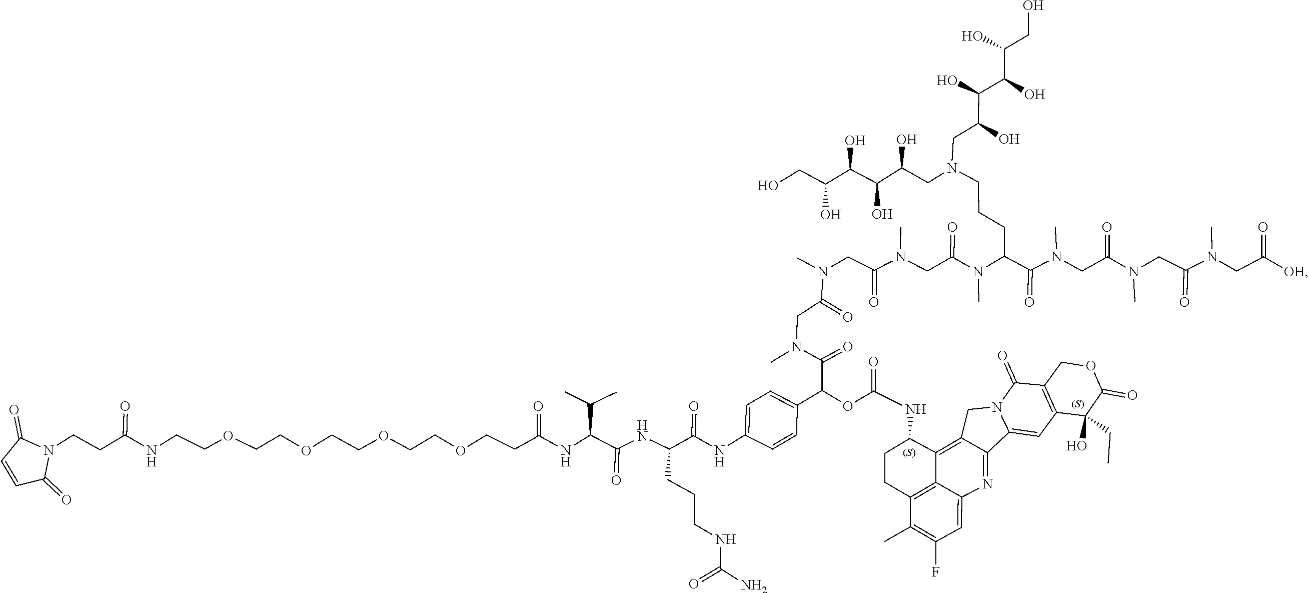

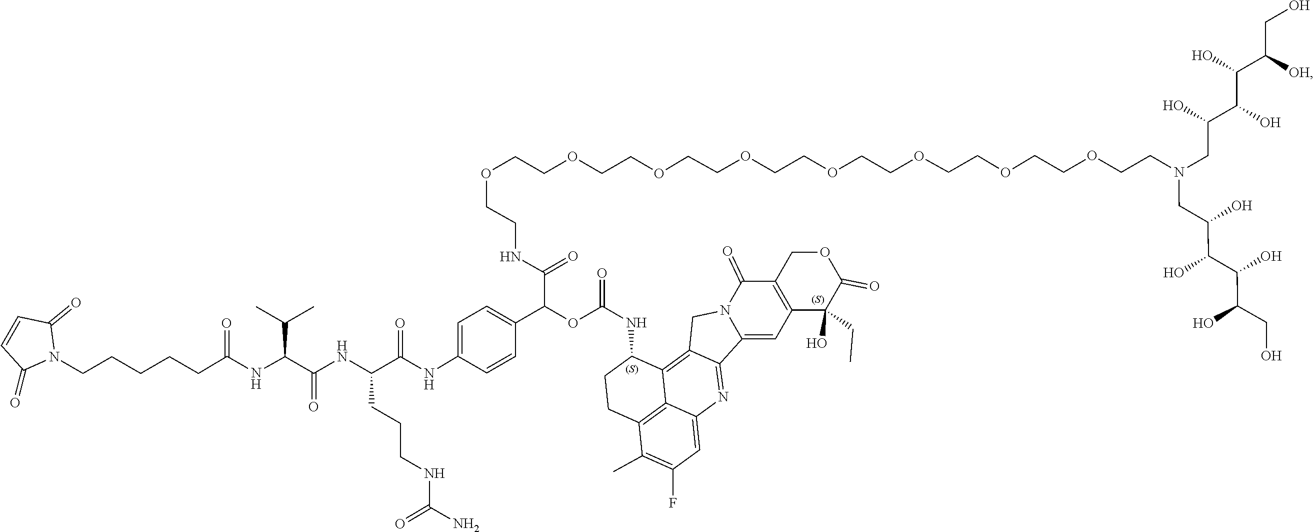

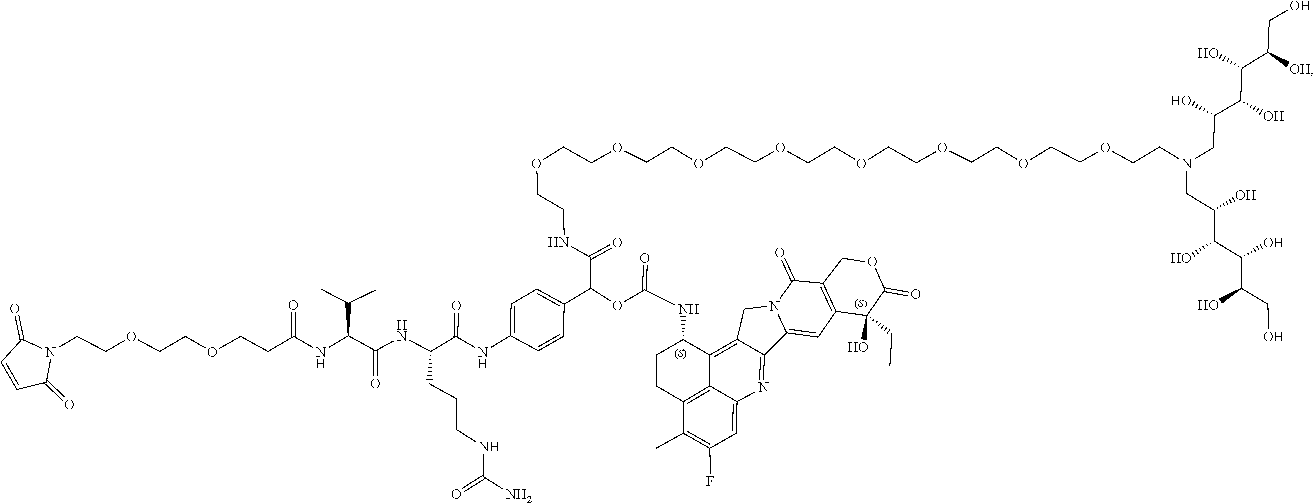

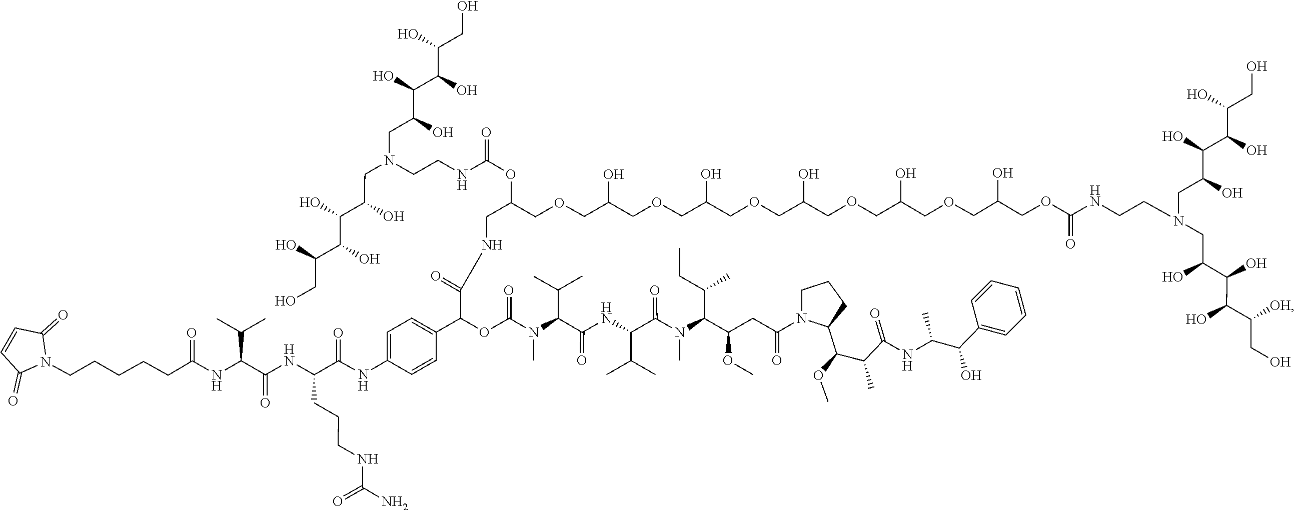 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)OC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

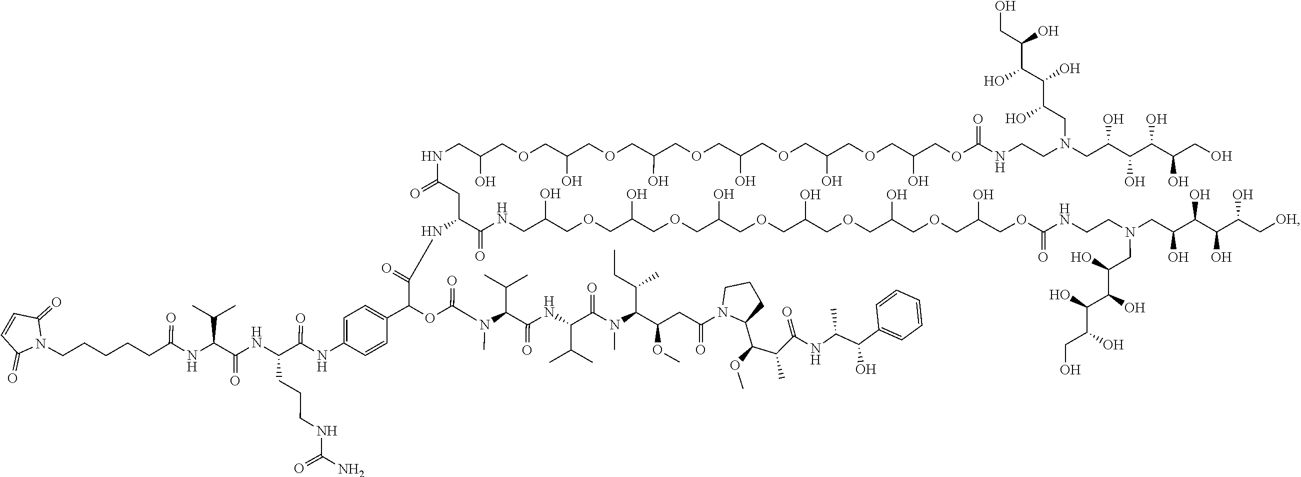 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

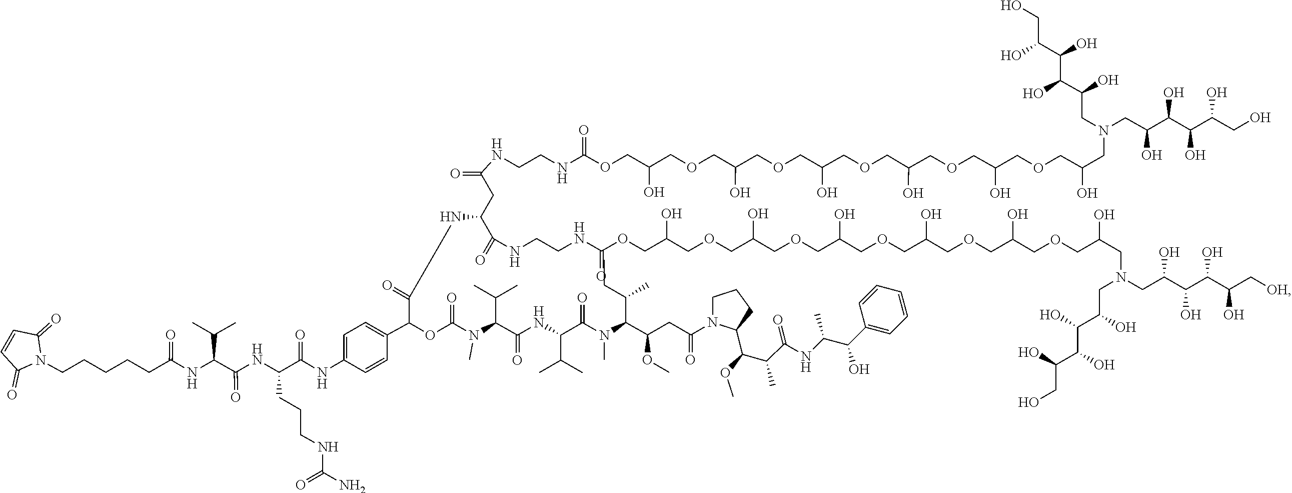 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

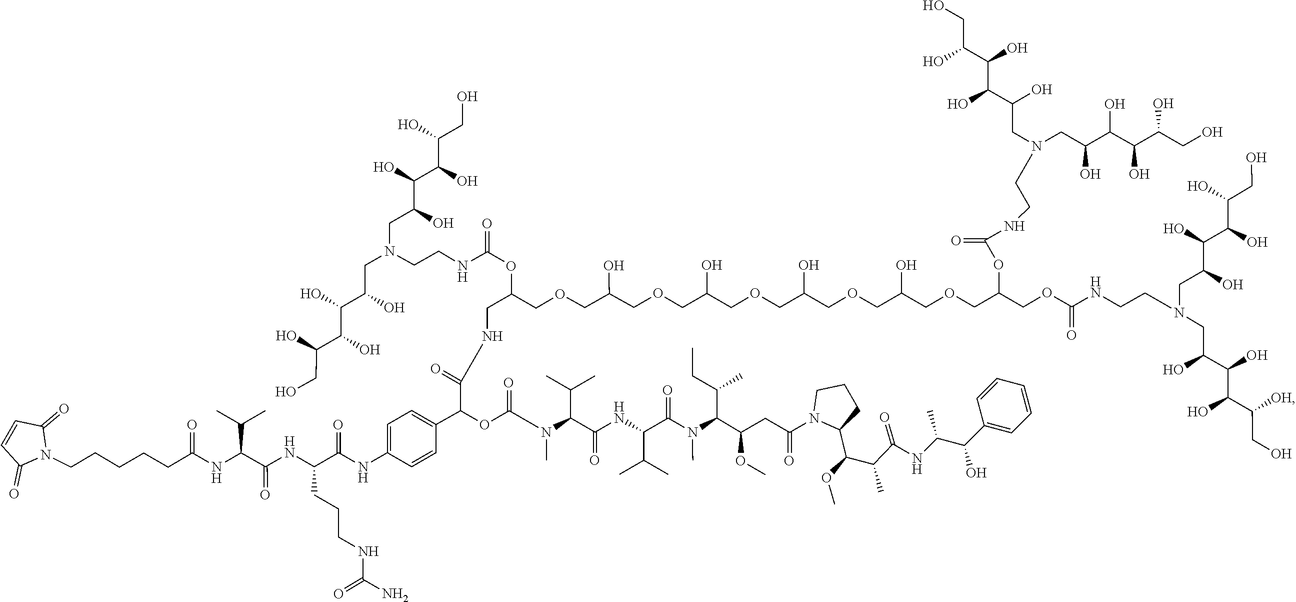

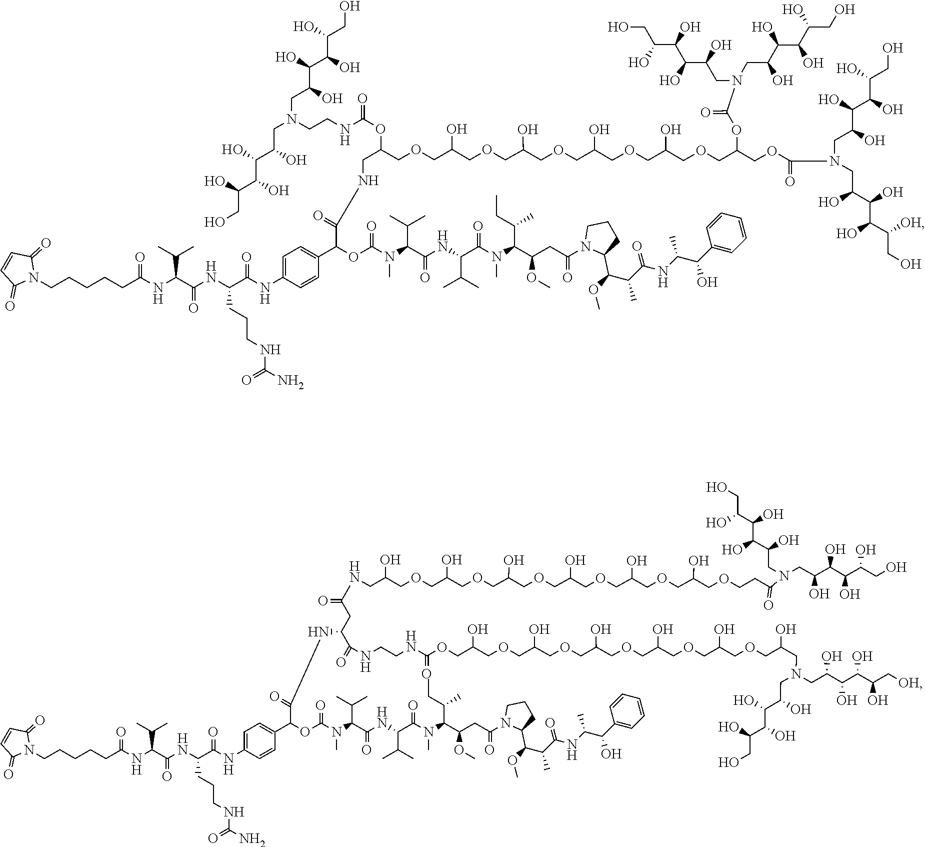

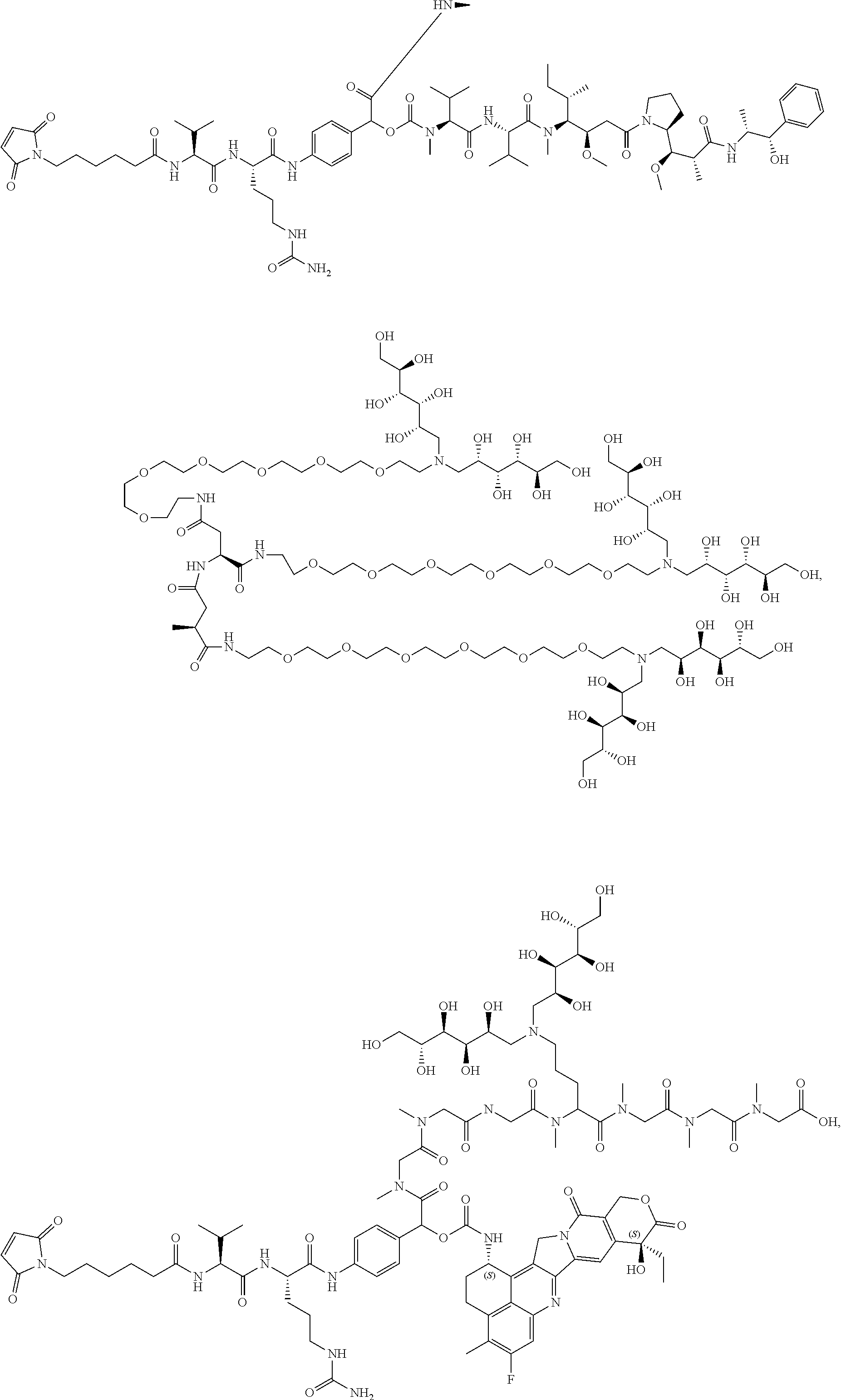

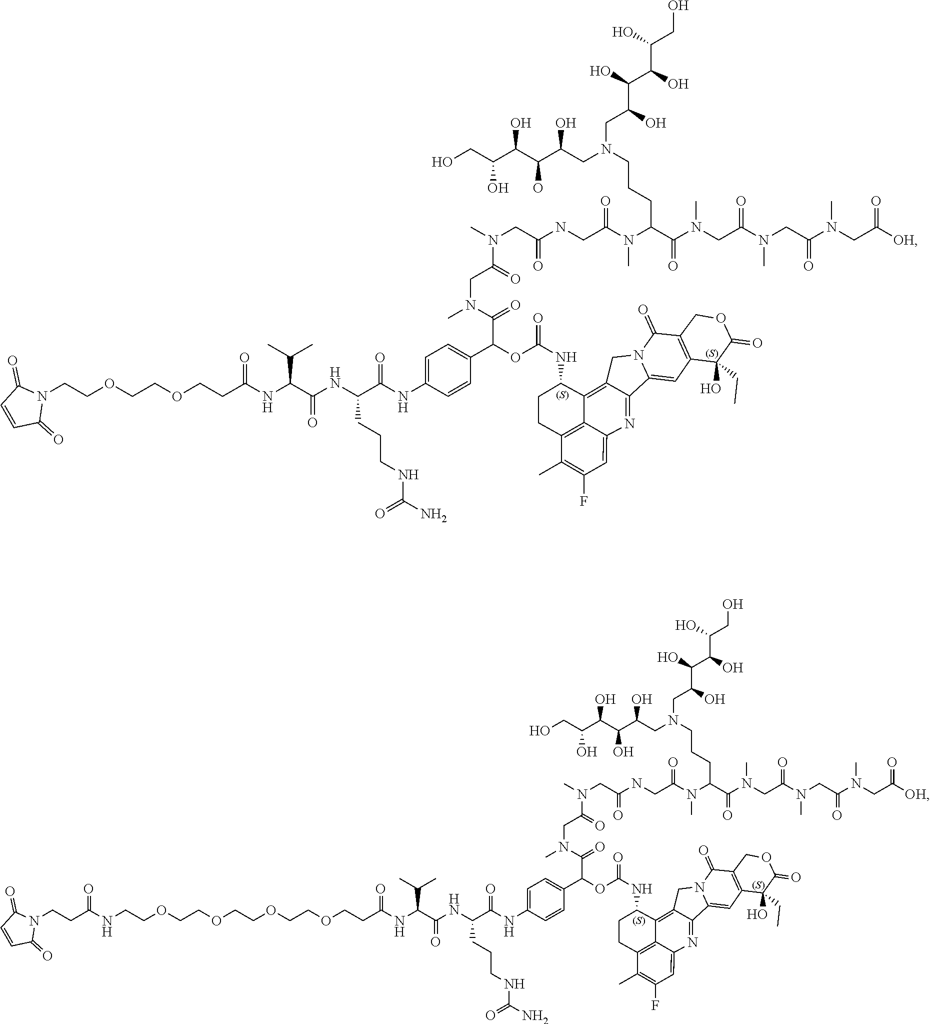

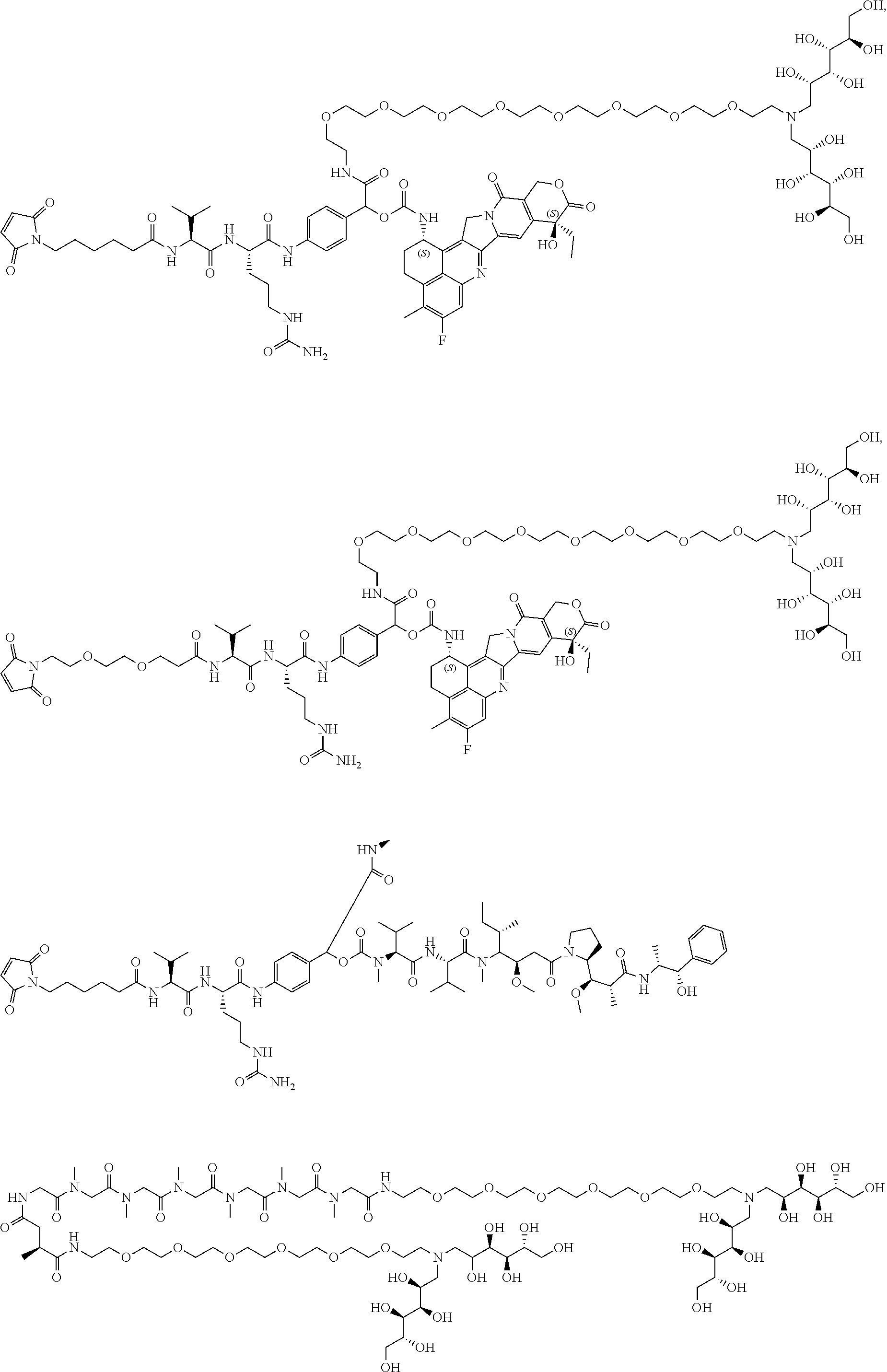

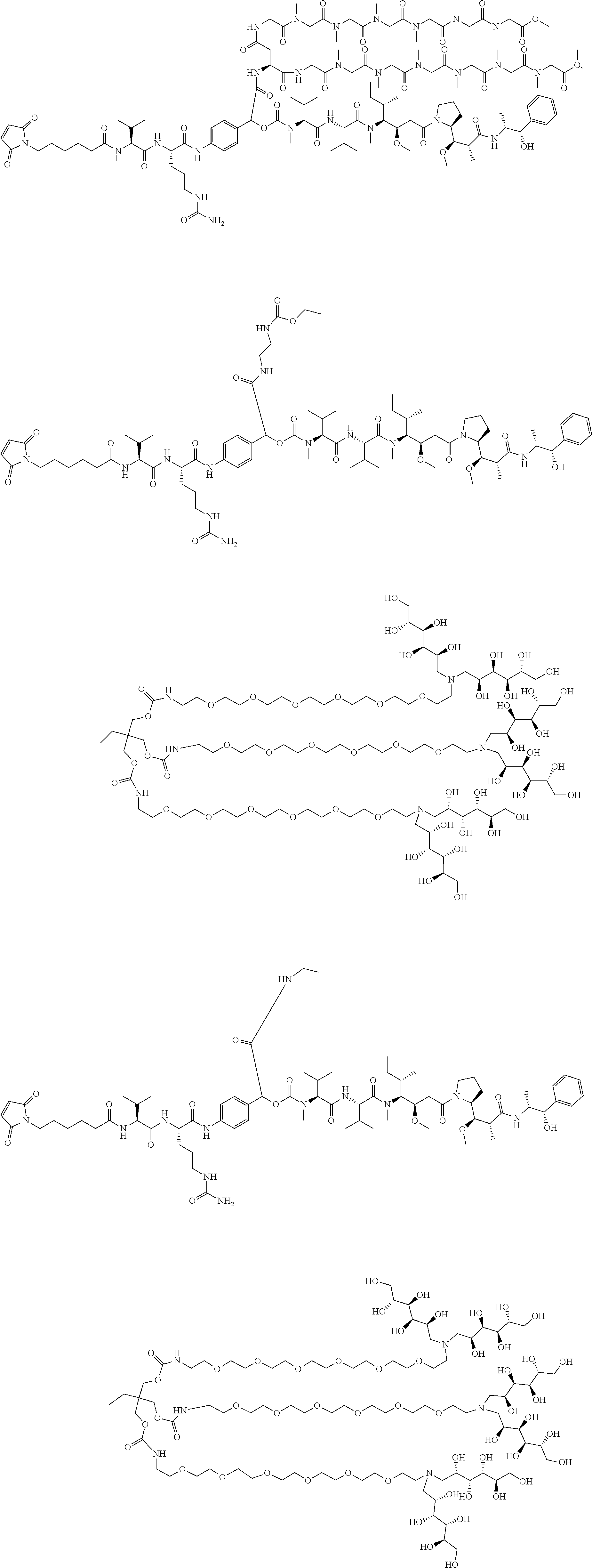

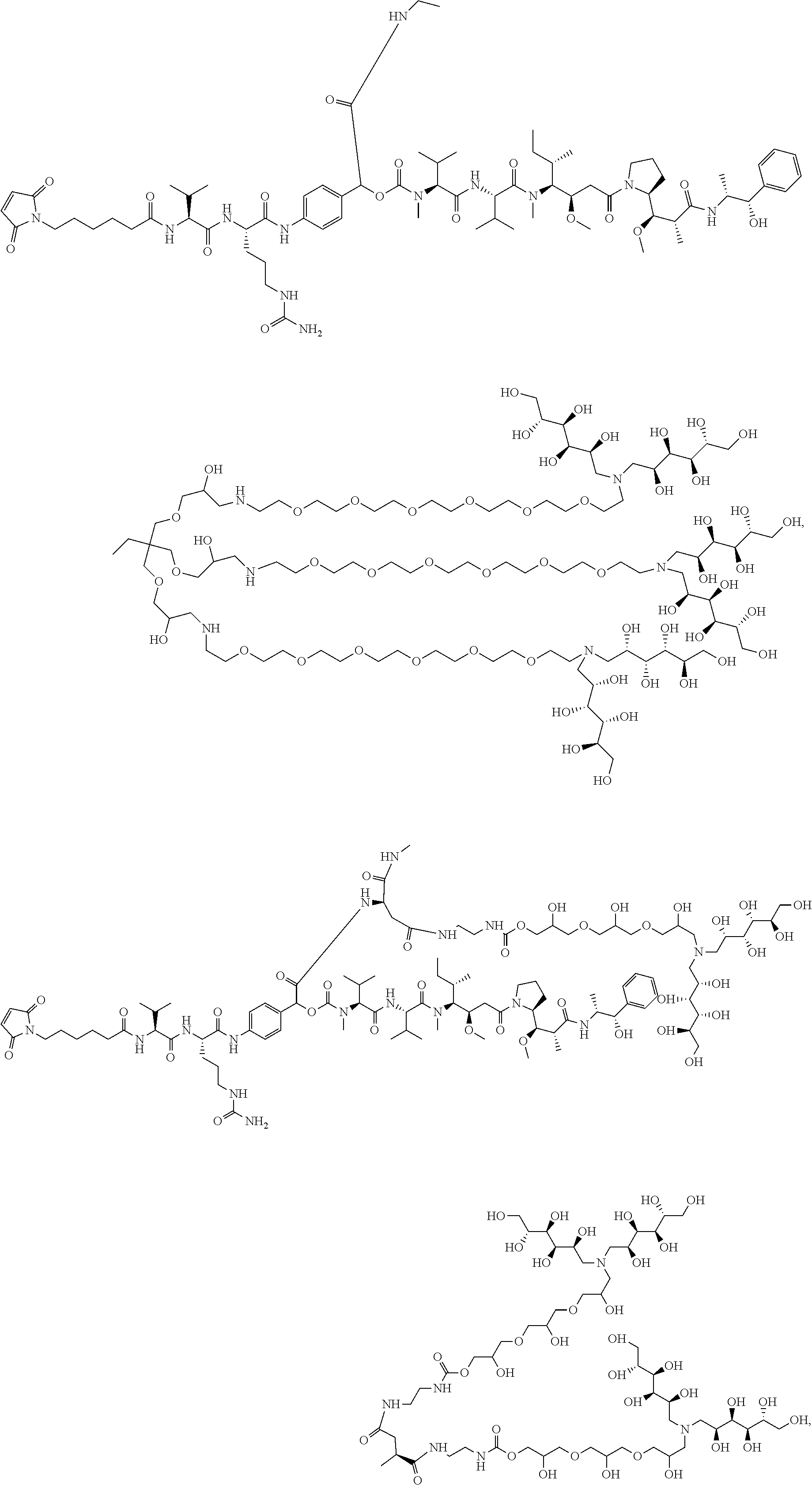

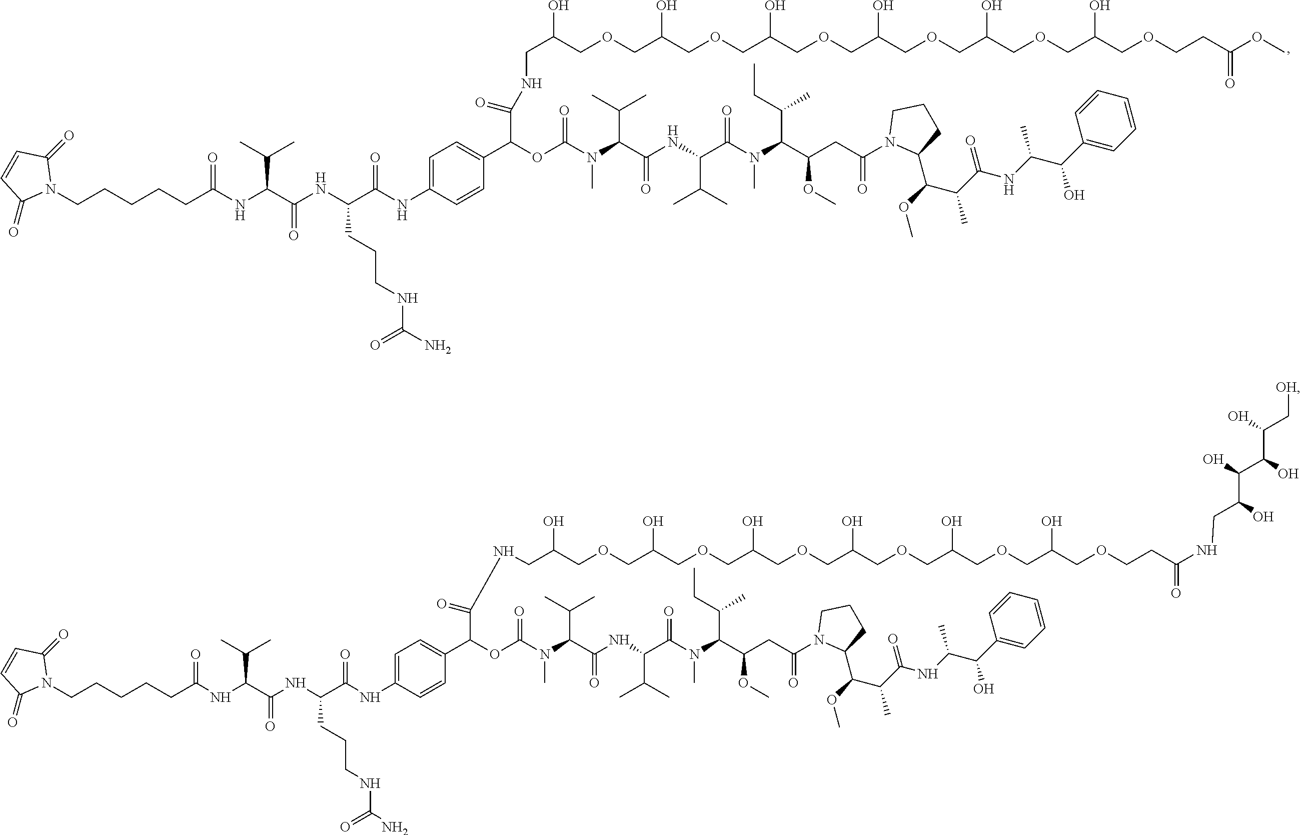

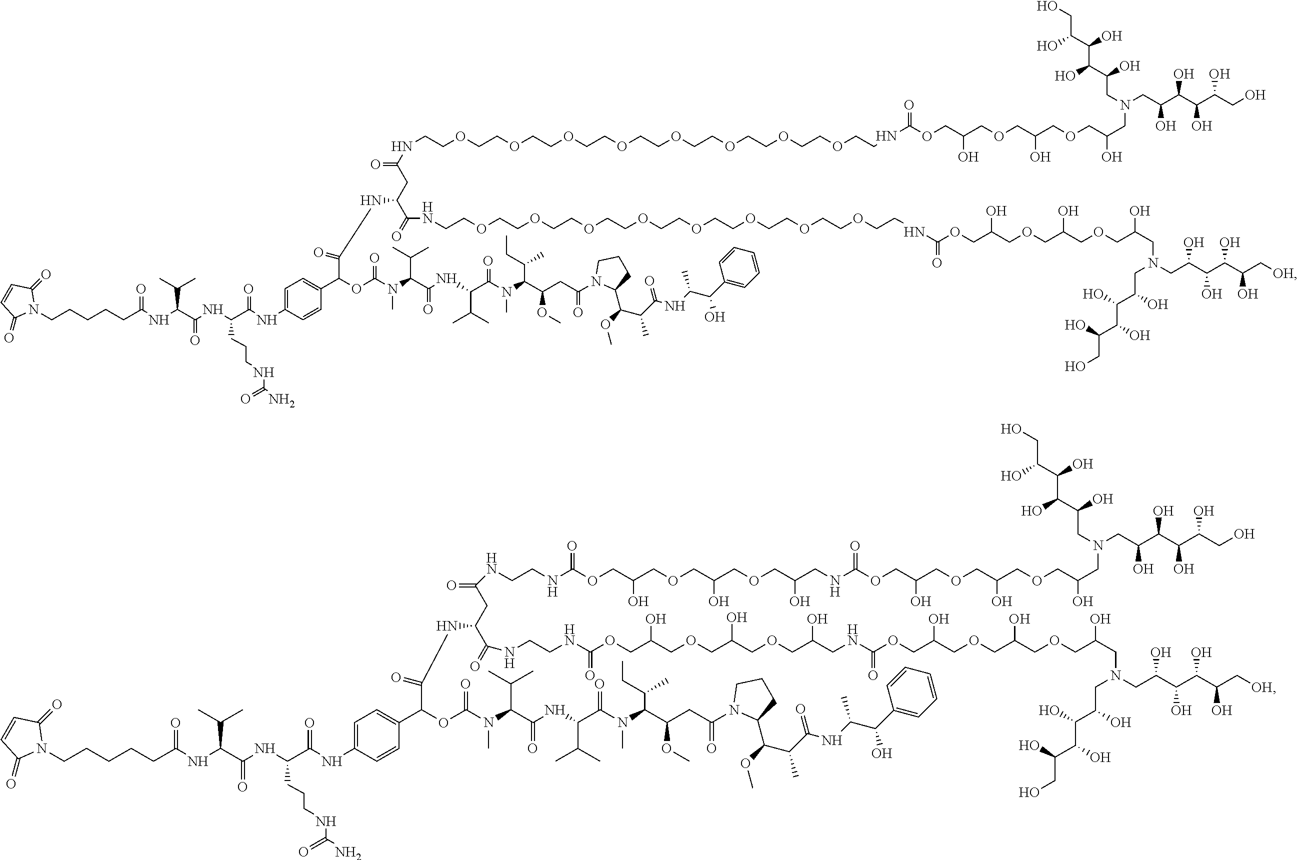

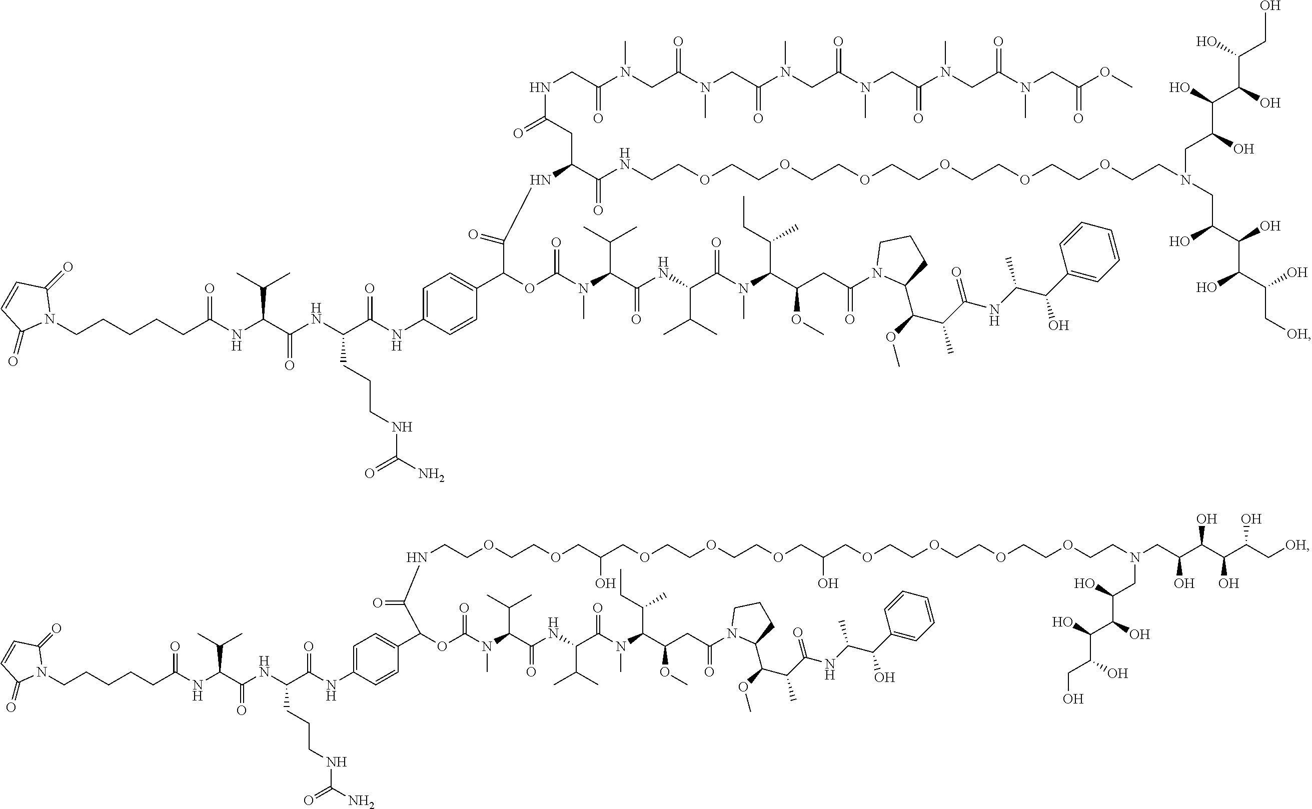

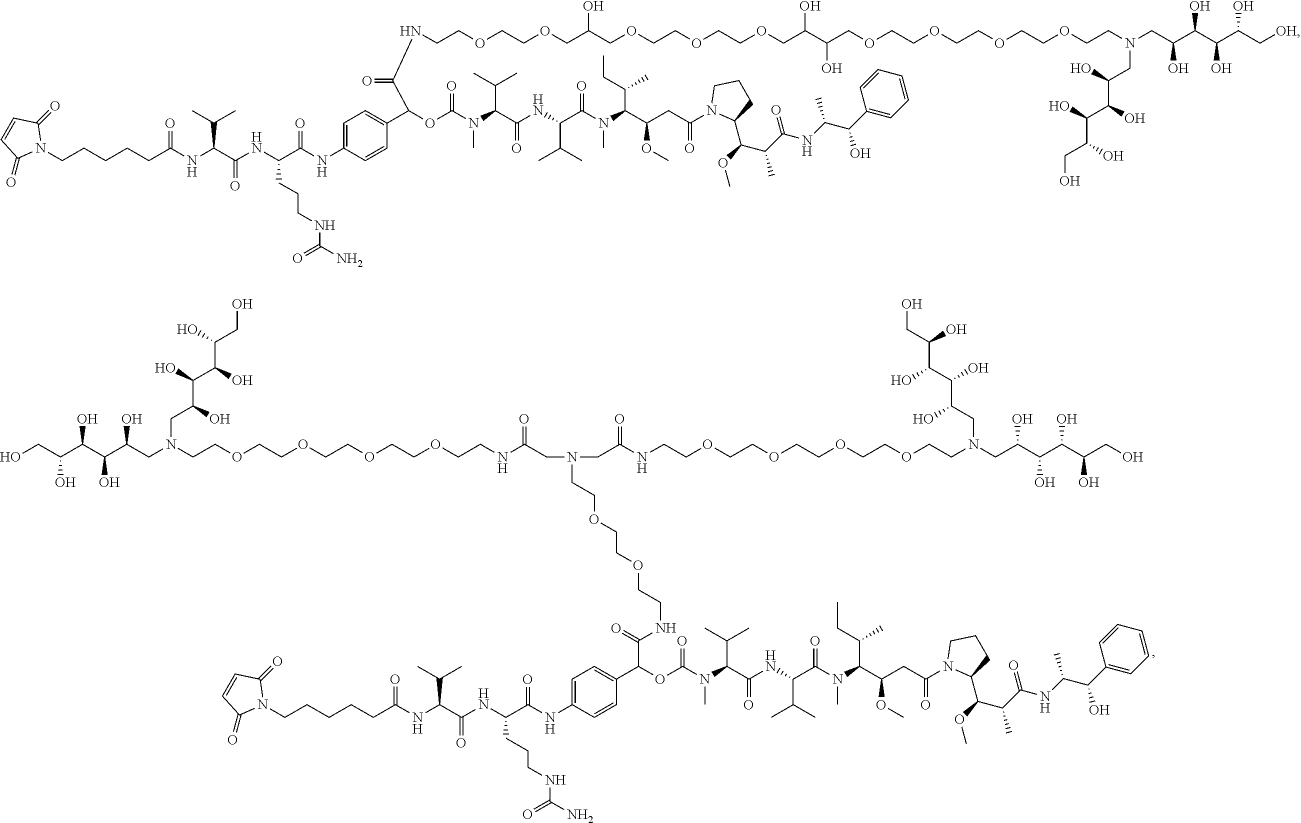

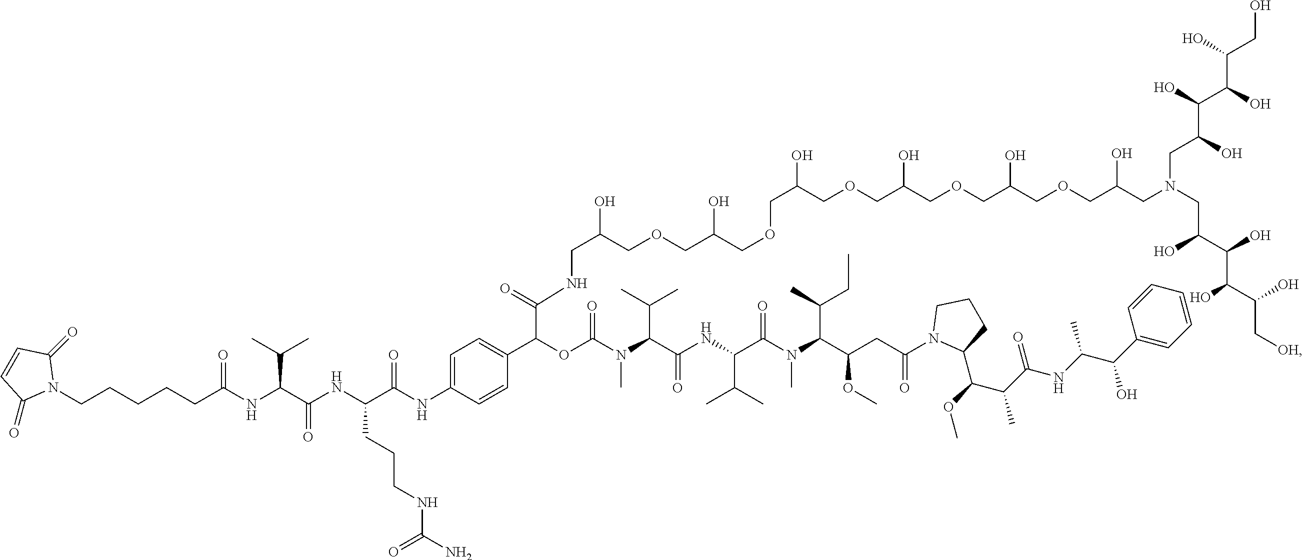 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

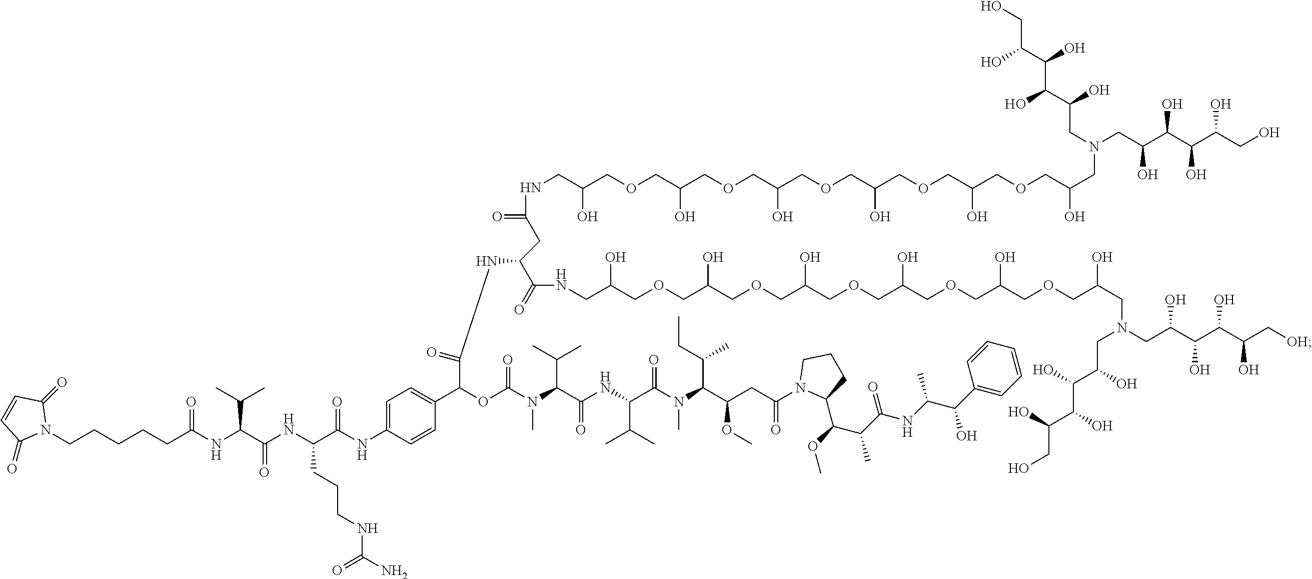 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

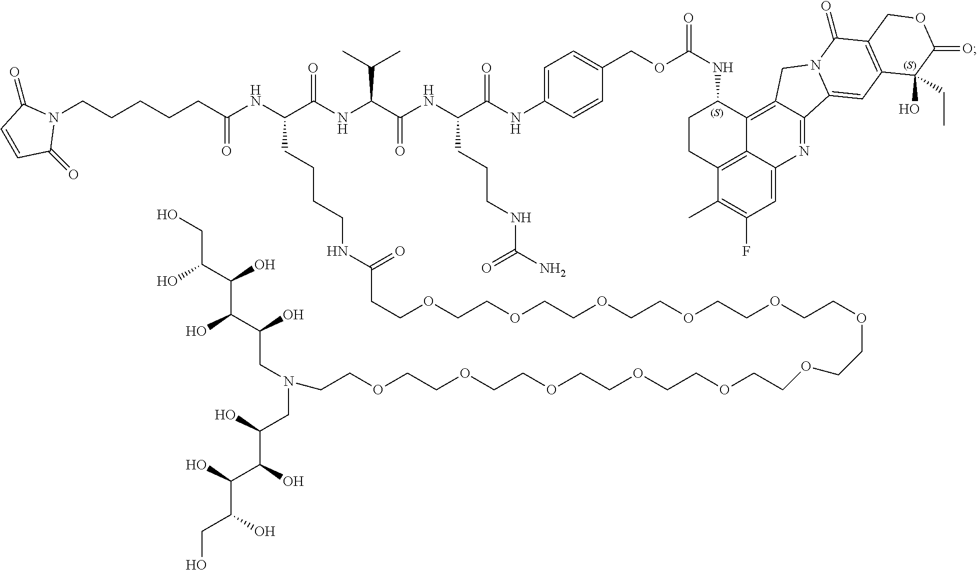

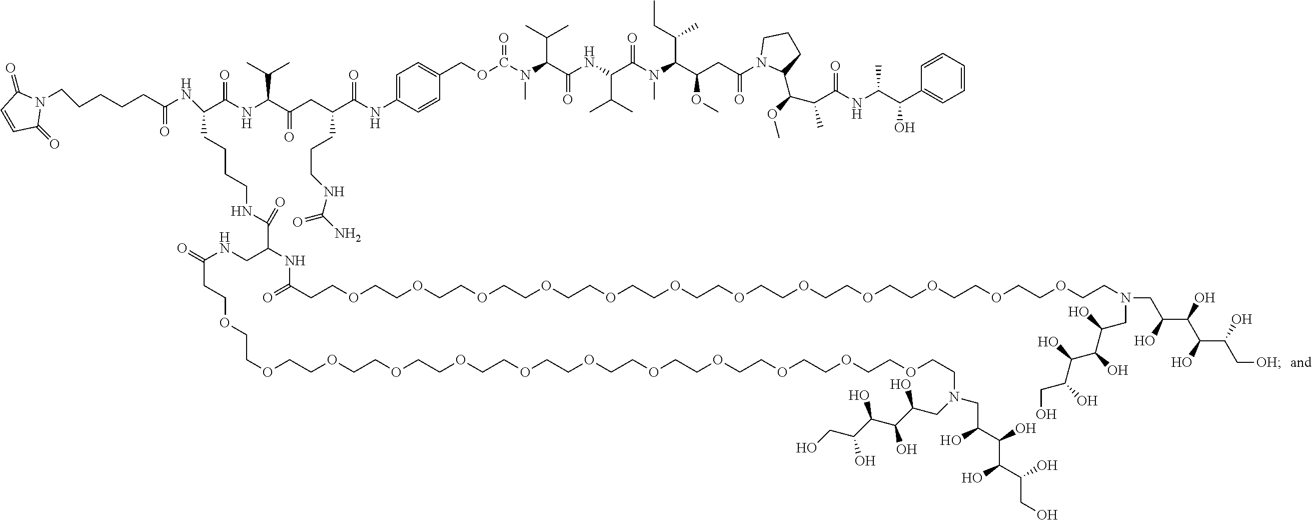

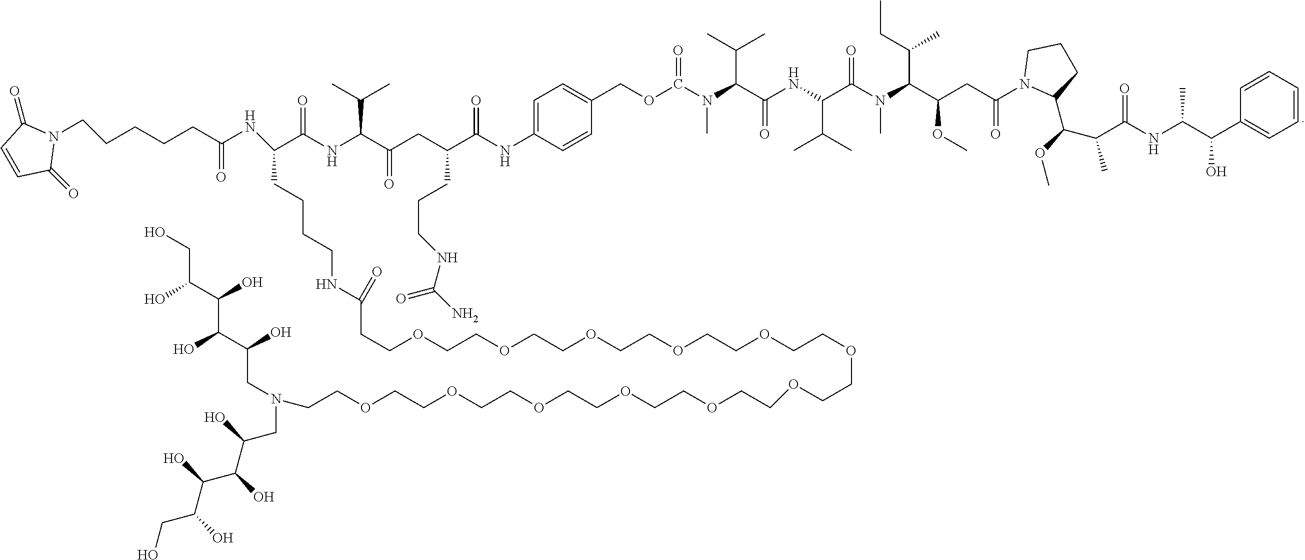

94. The conjugate of claim 26, wherein the average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

95. (canceled)

96. The conjugate of claim 26, selected from the following: 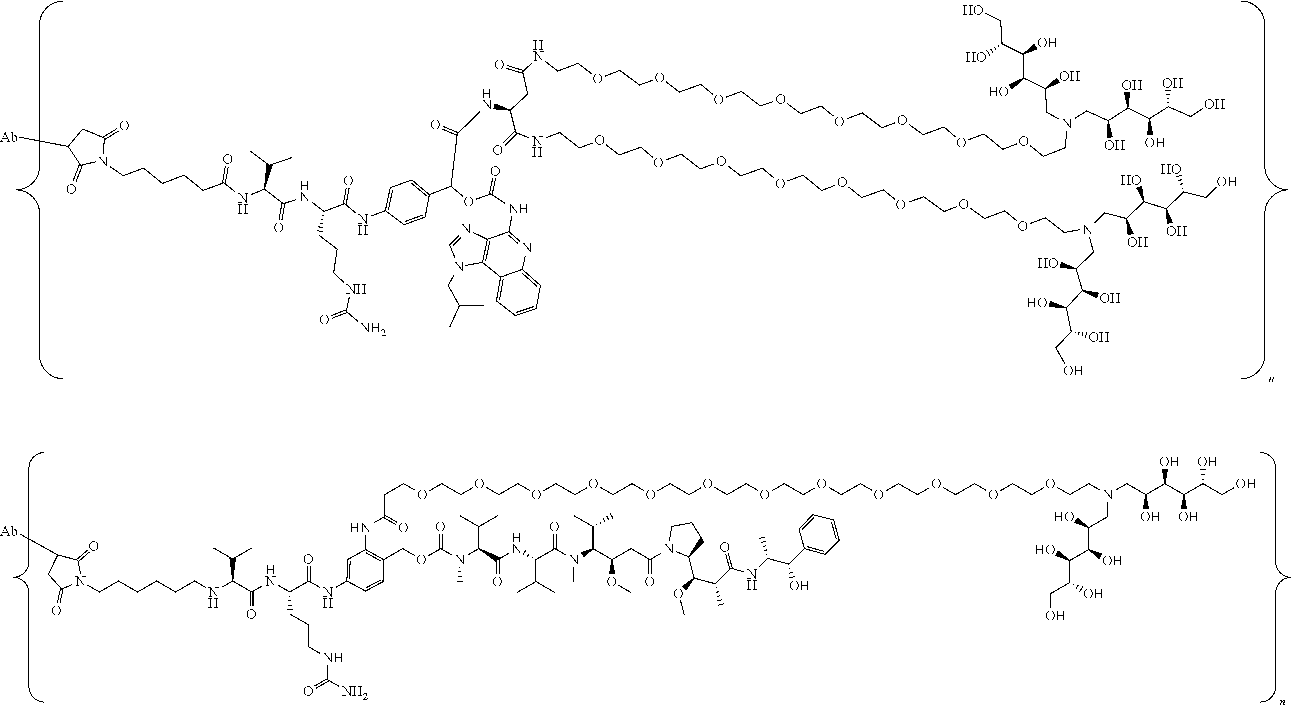 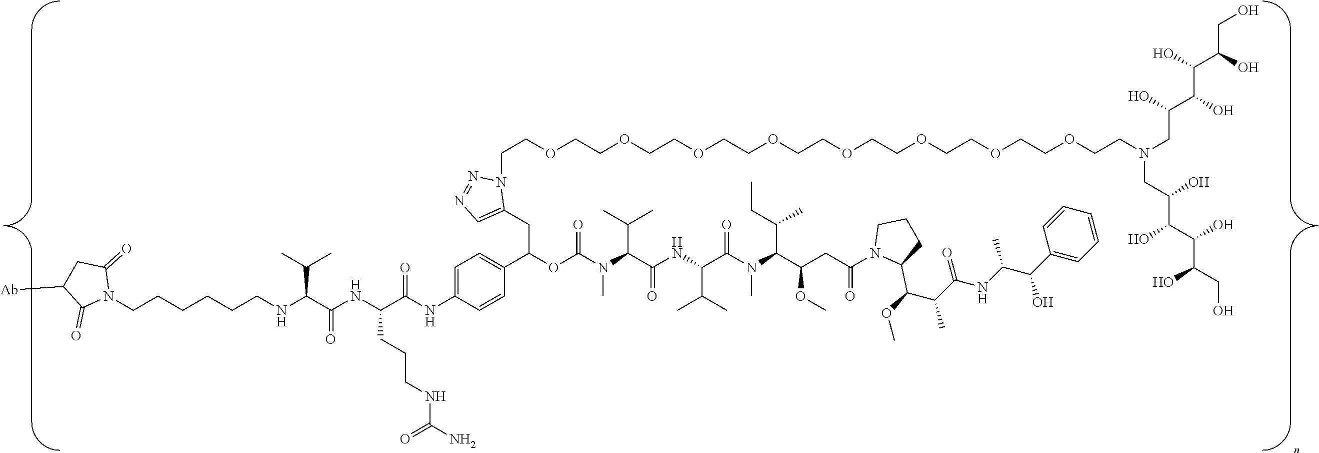 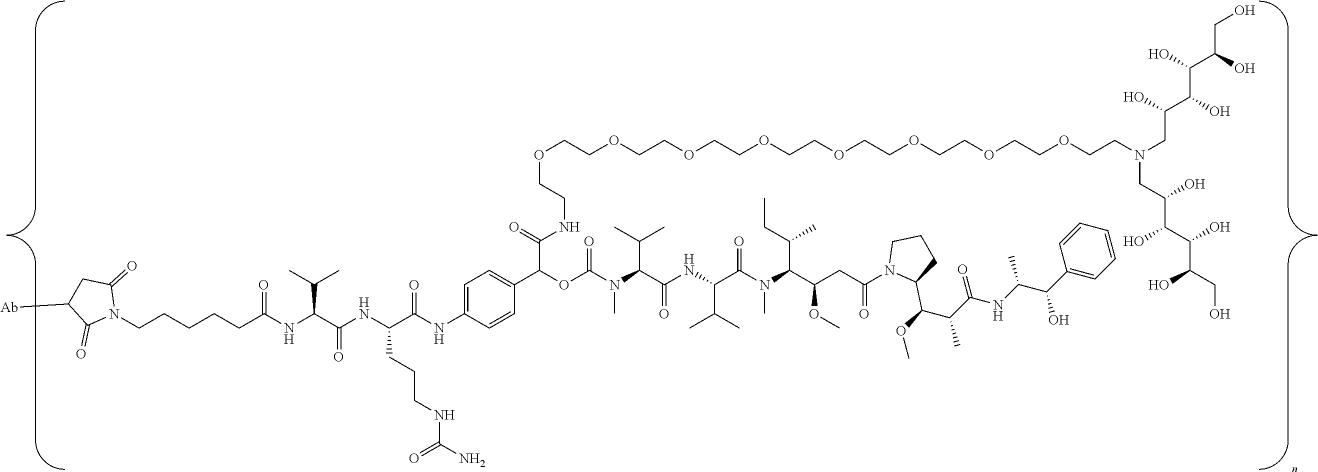 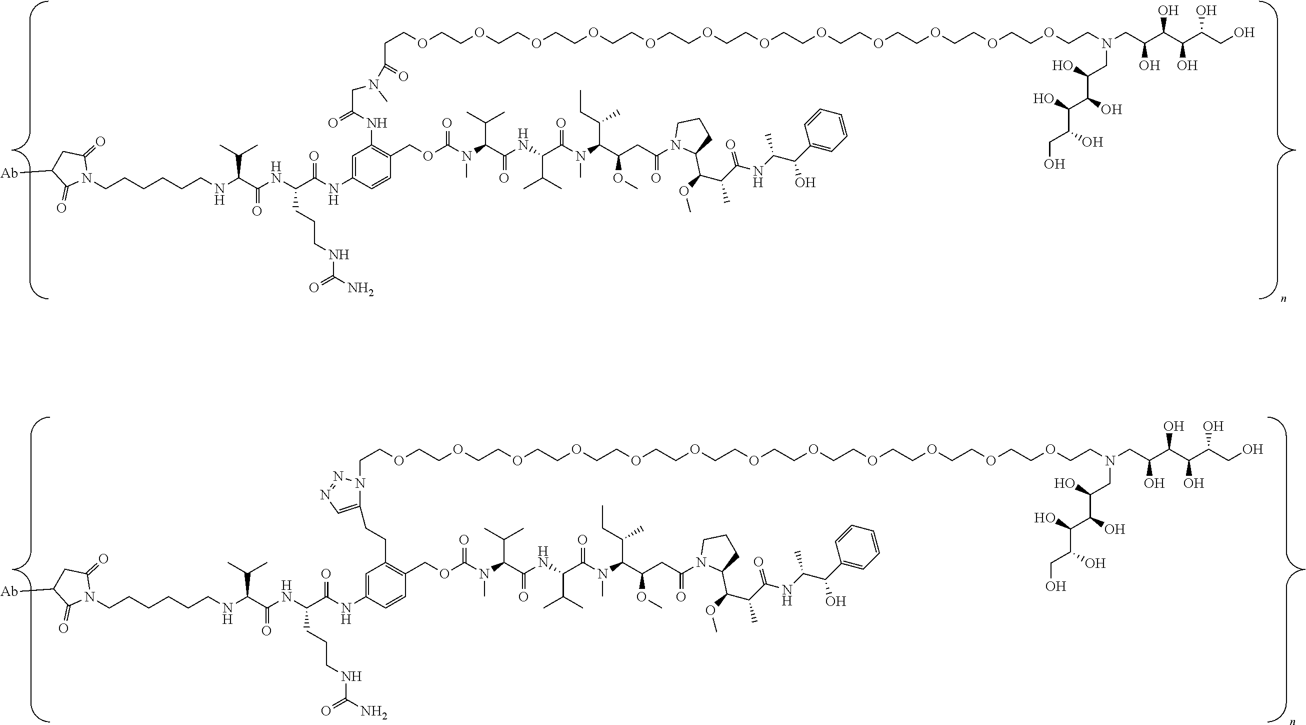 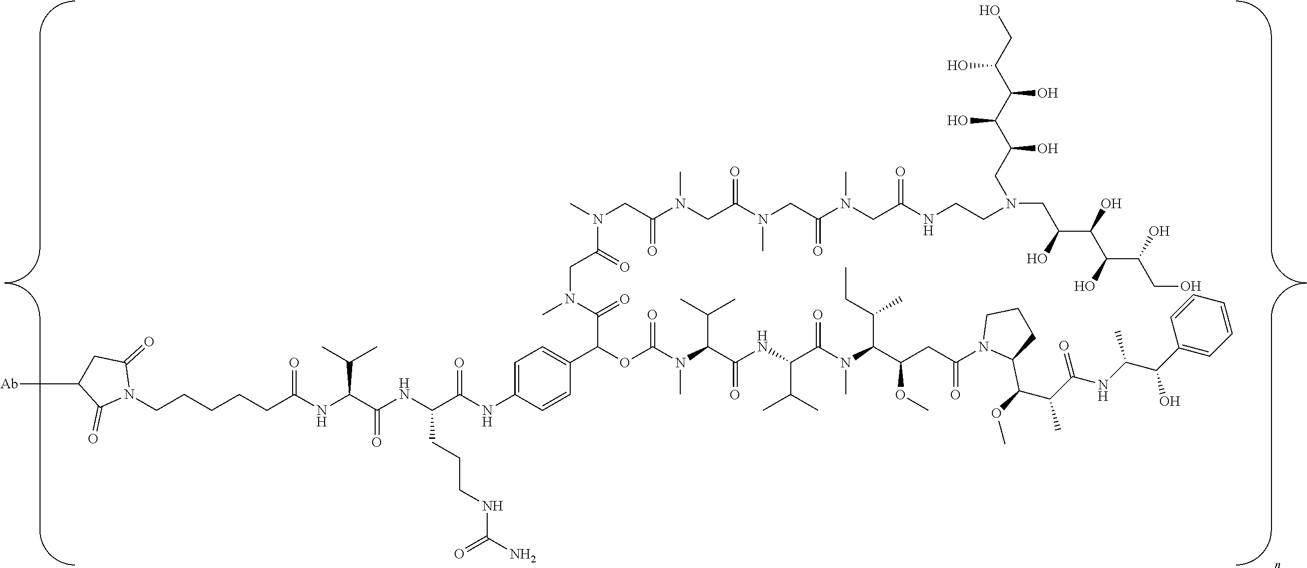 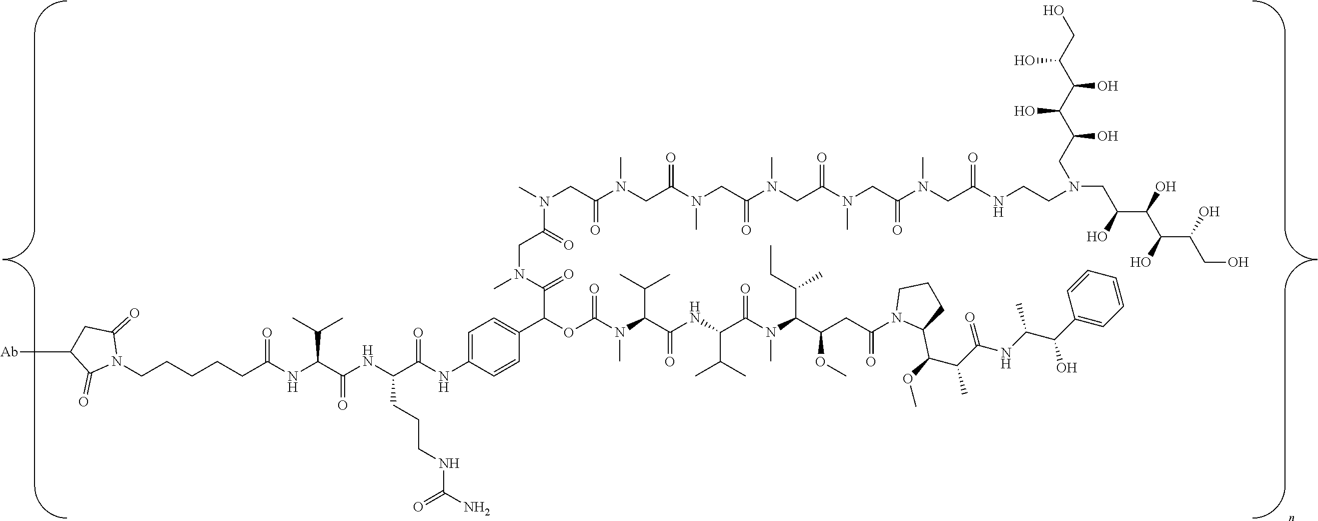 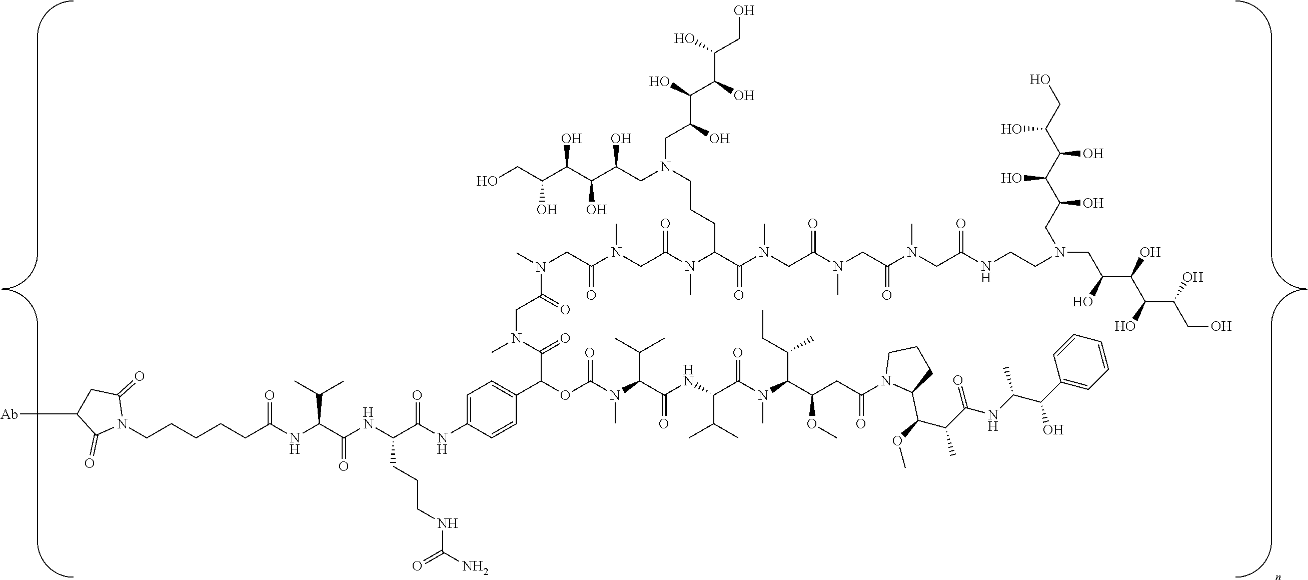 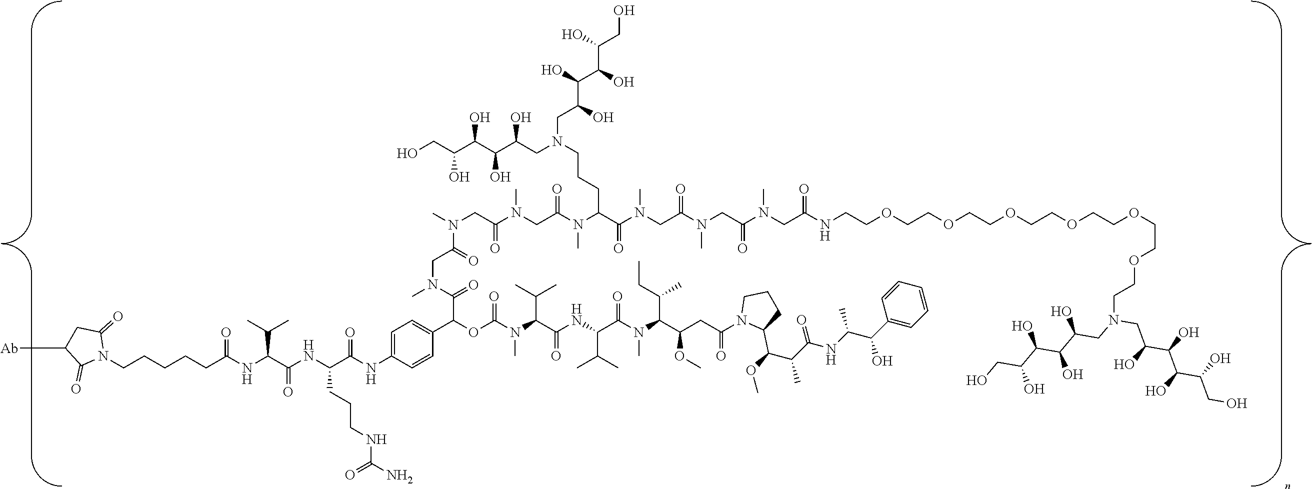 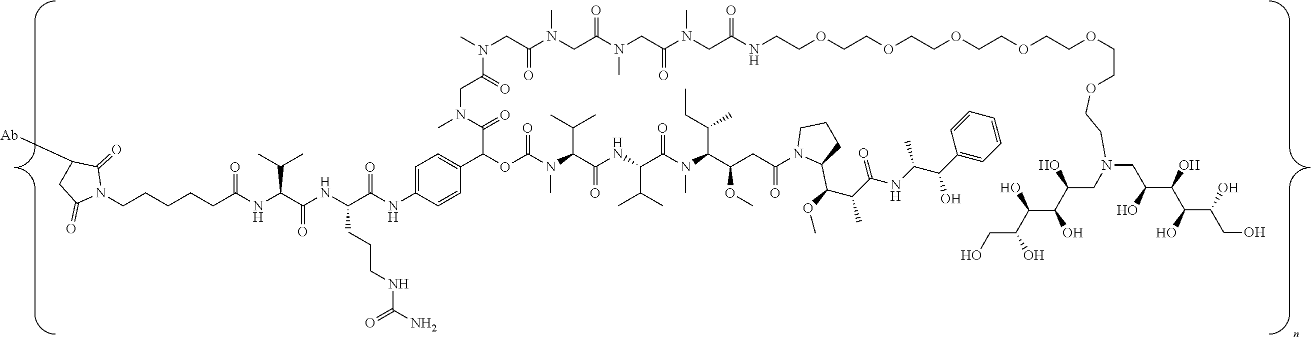 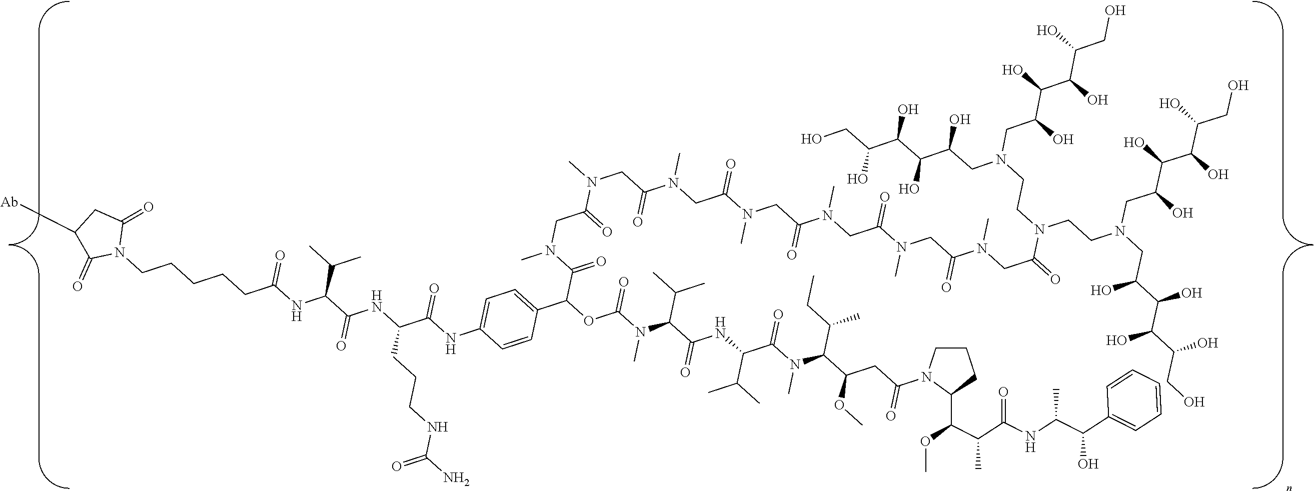 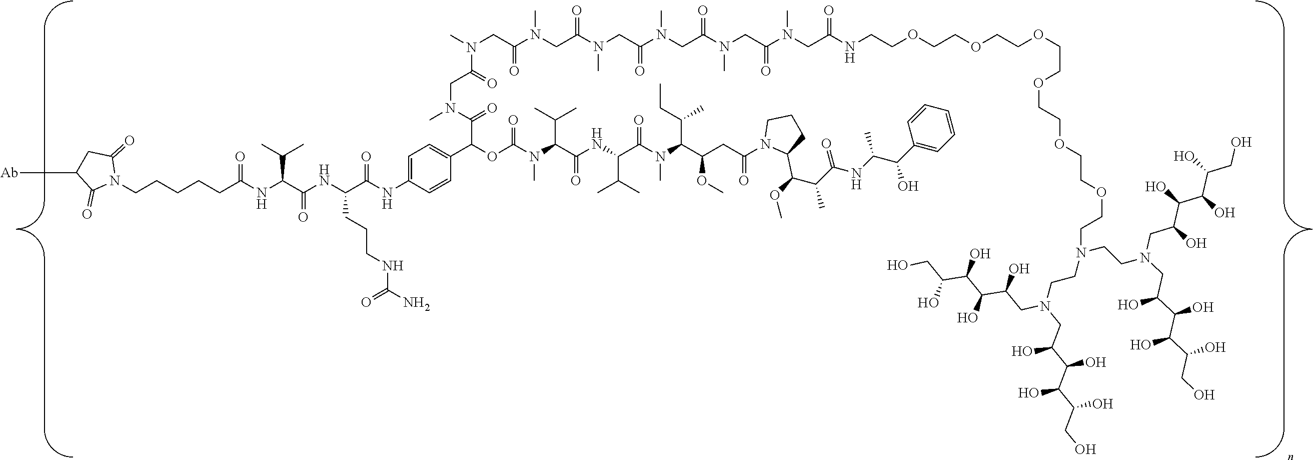 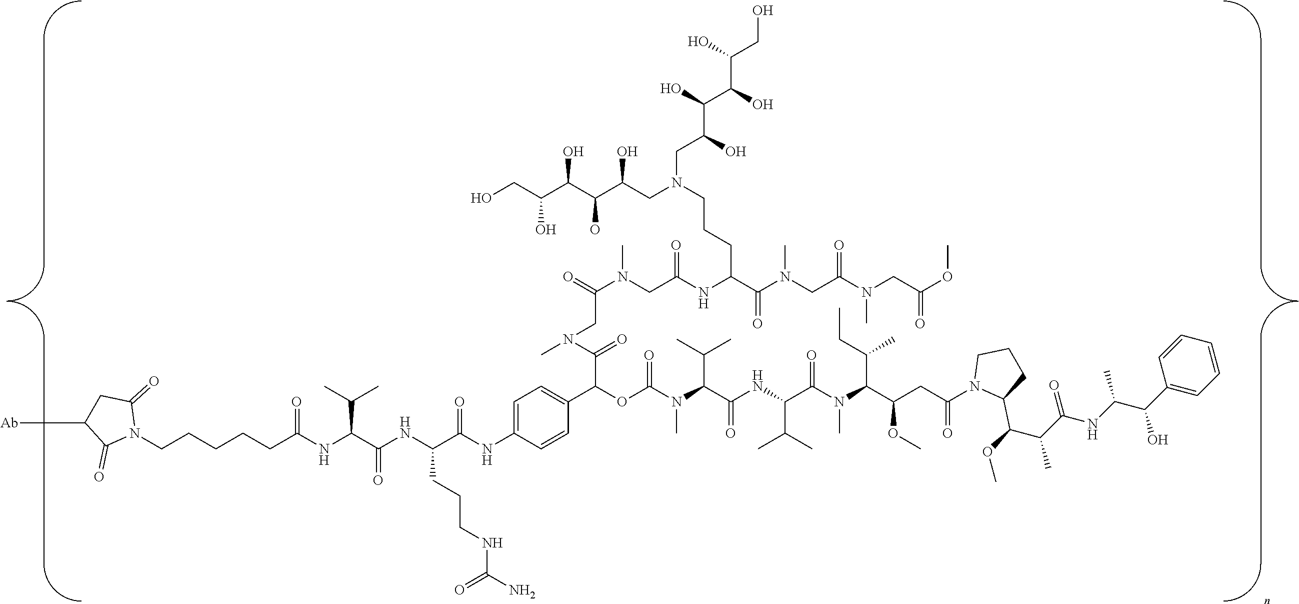 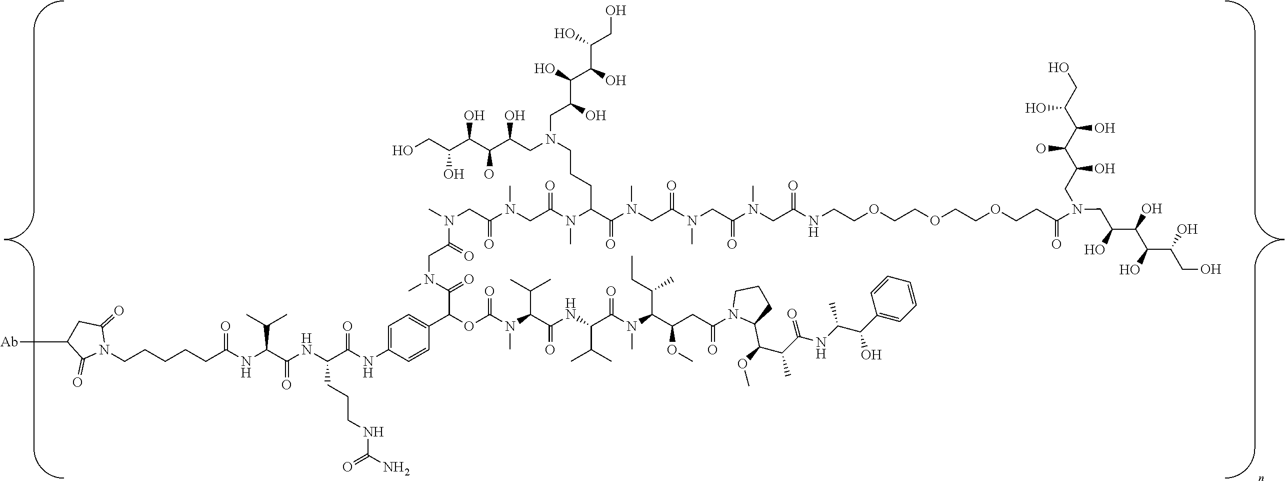 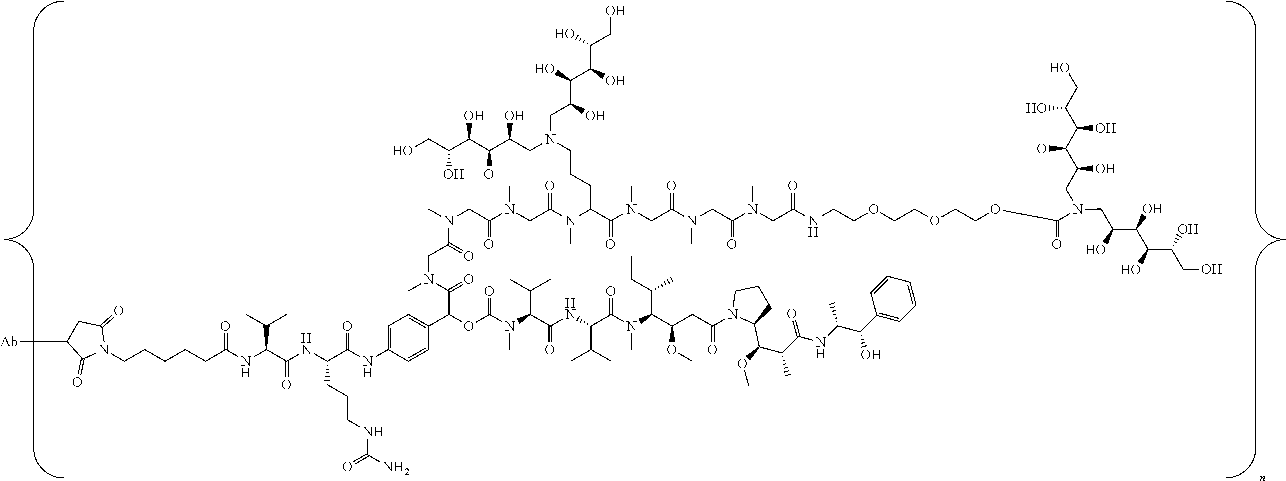 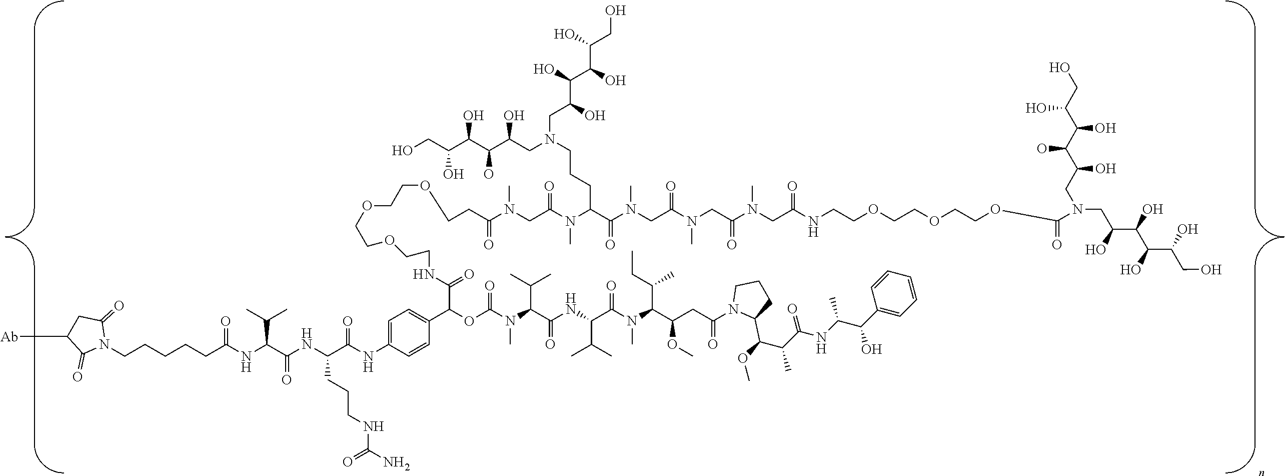 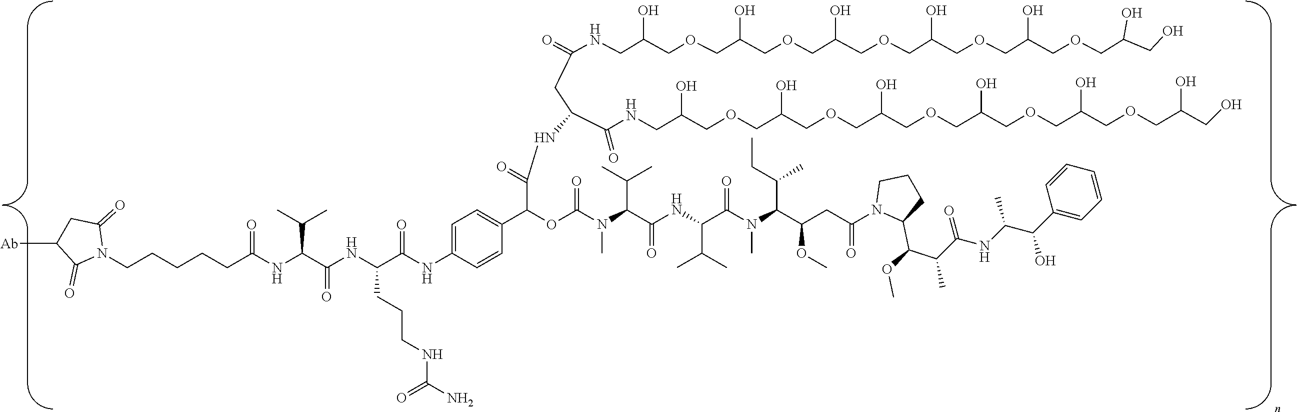 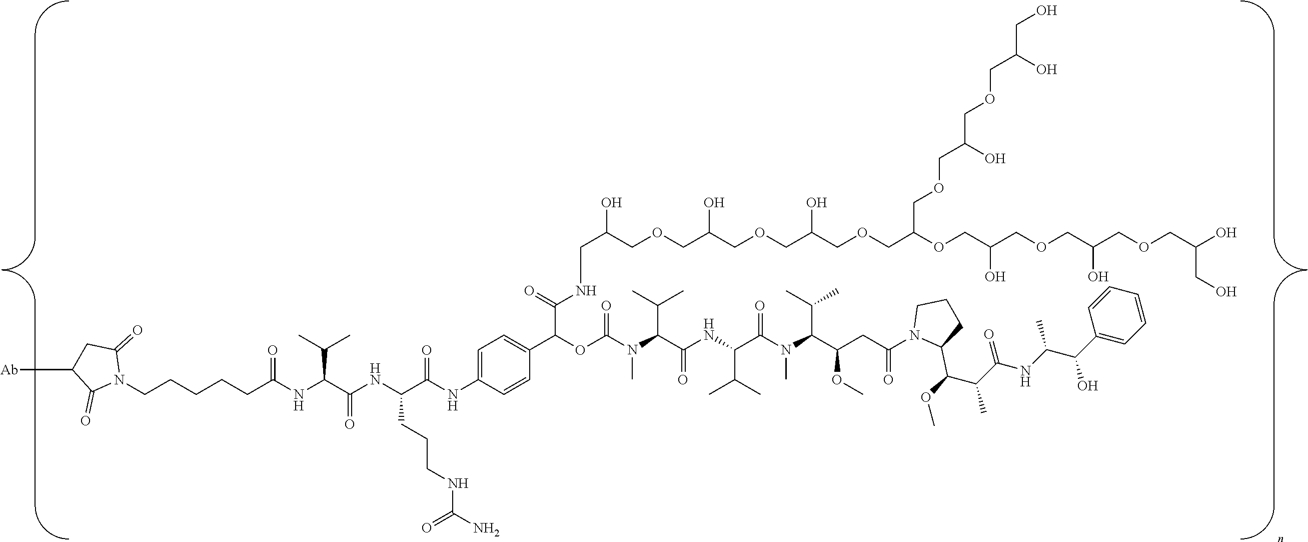 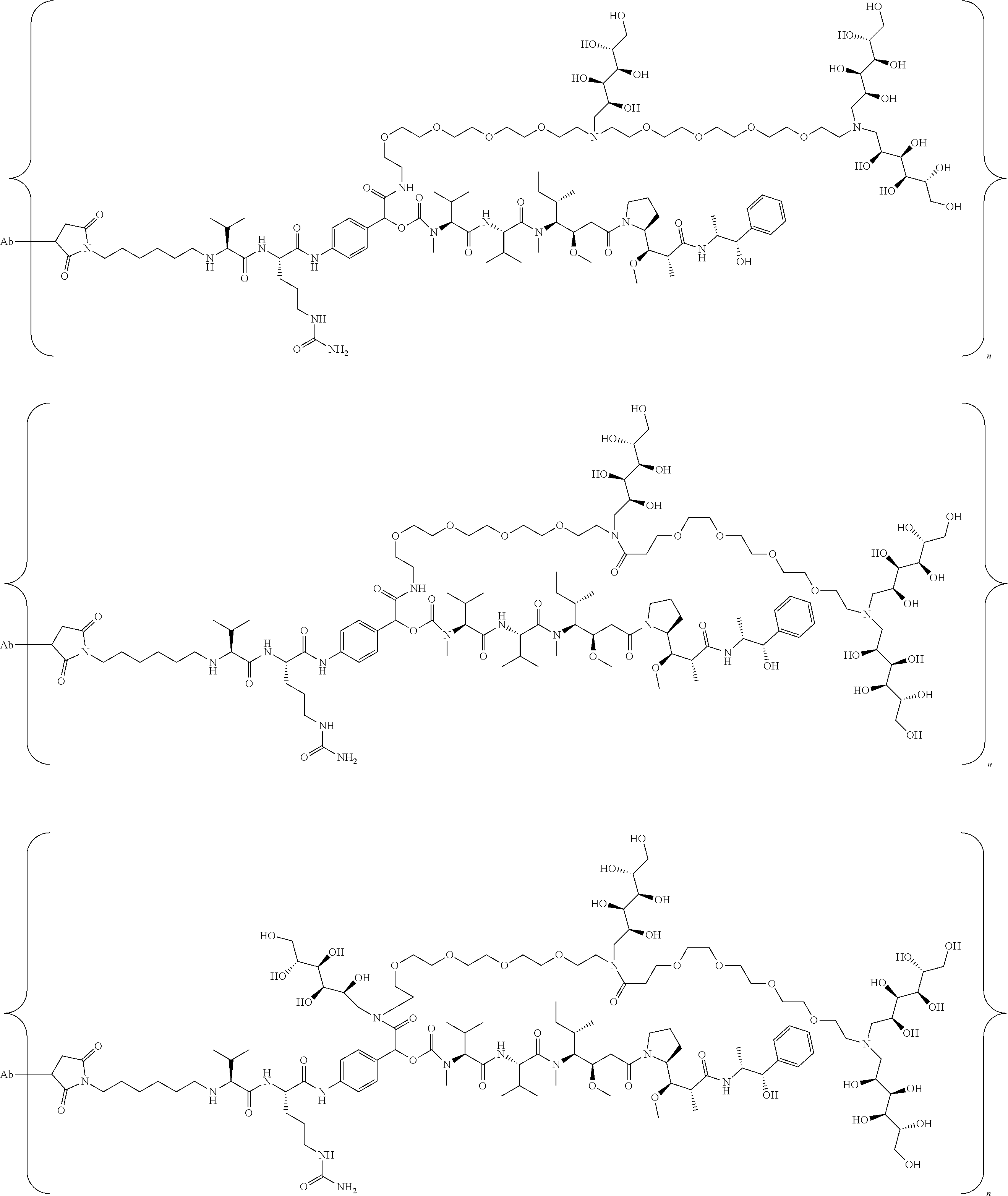 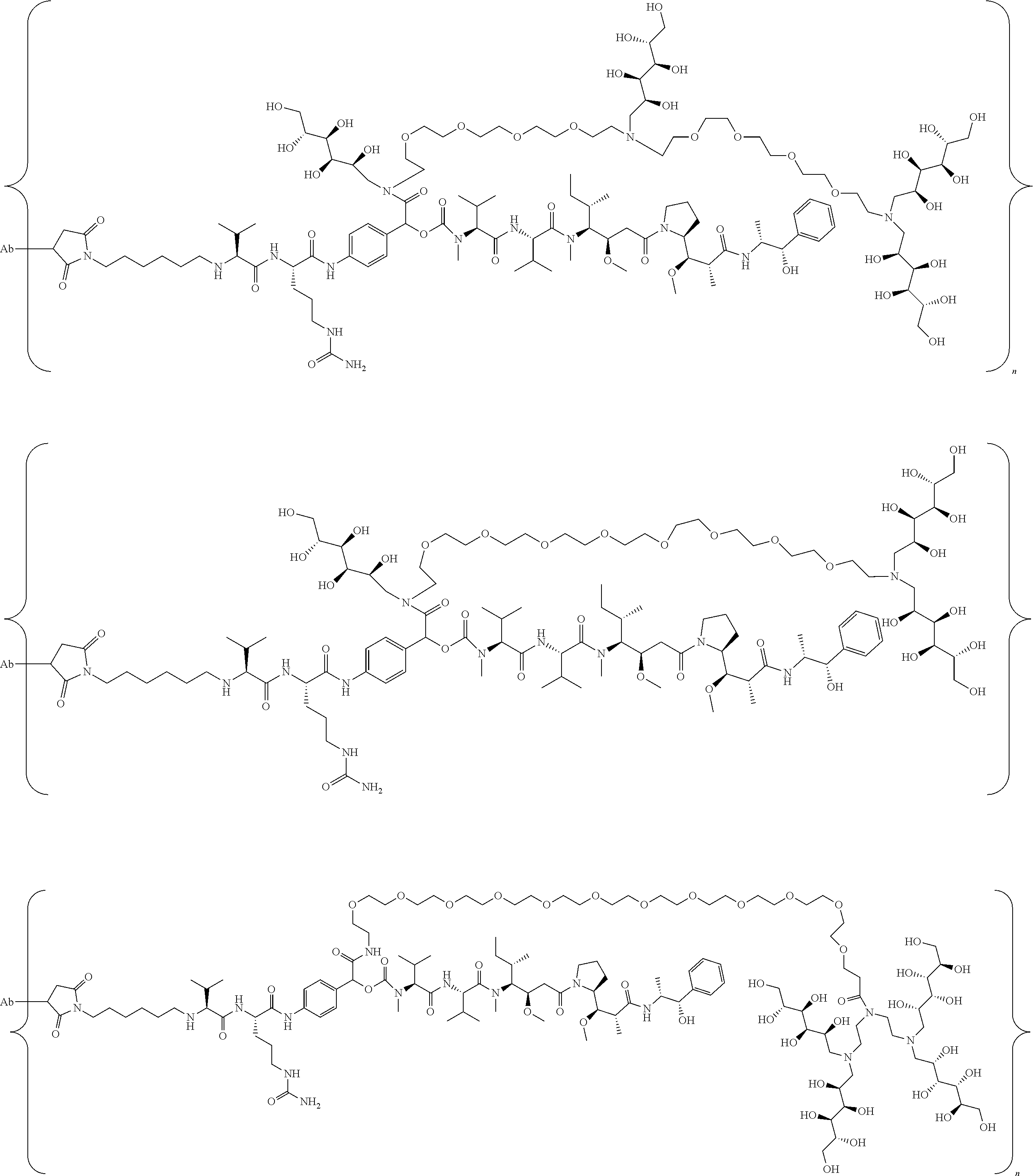 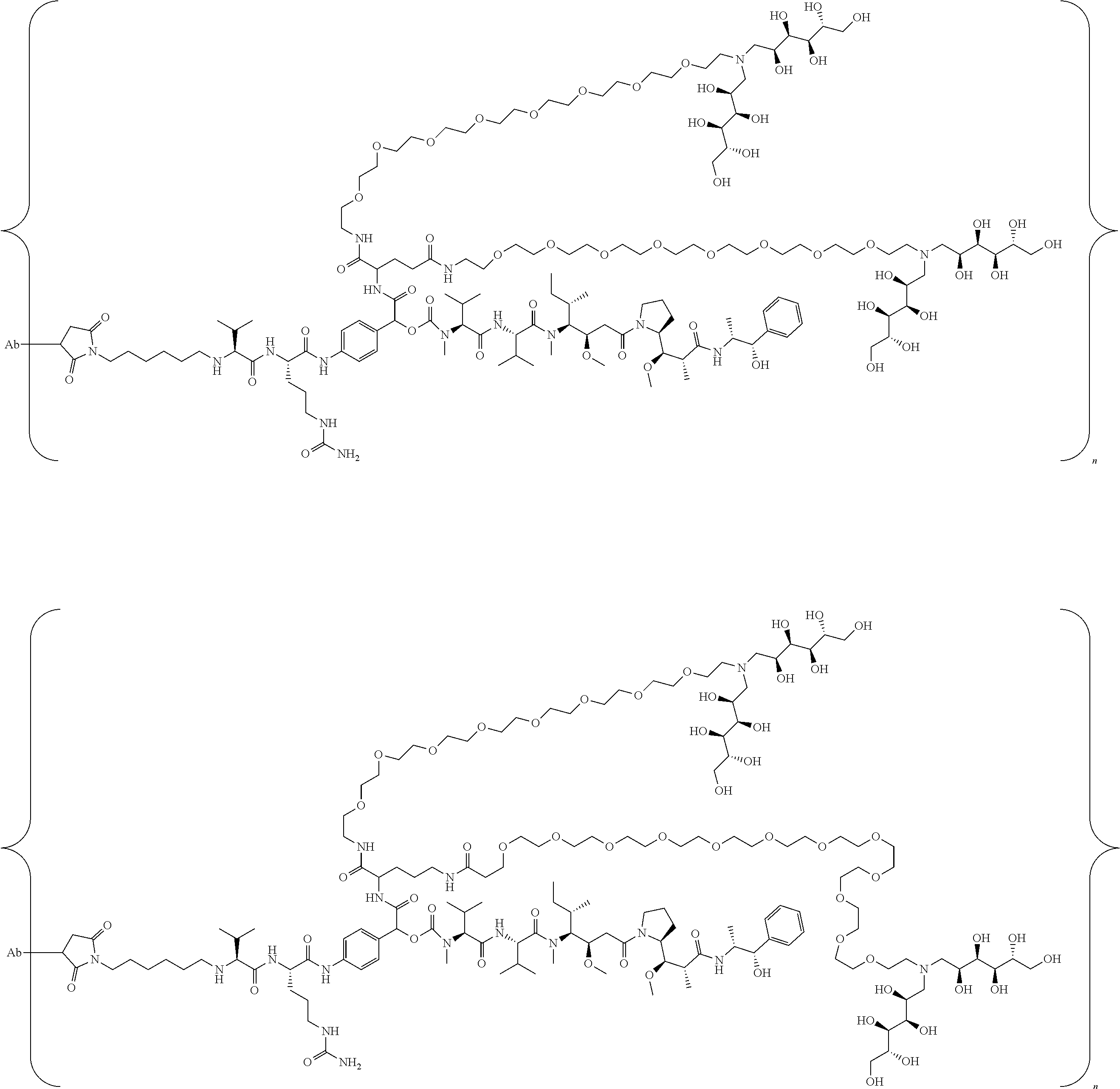 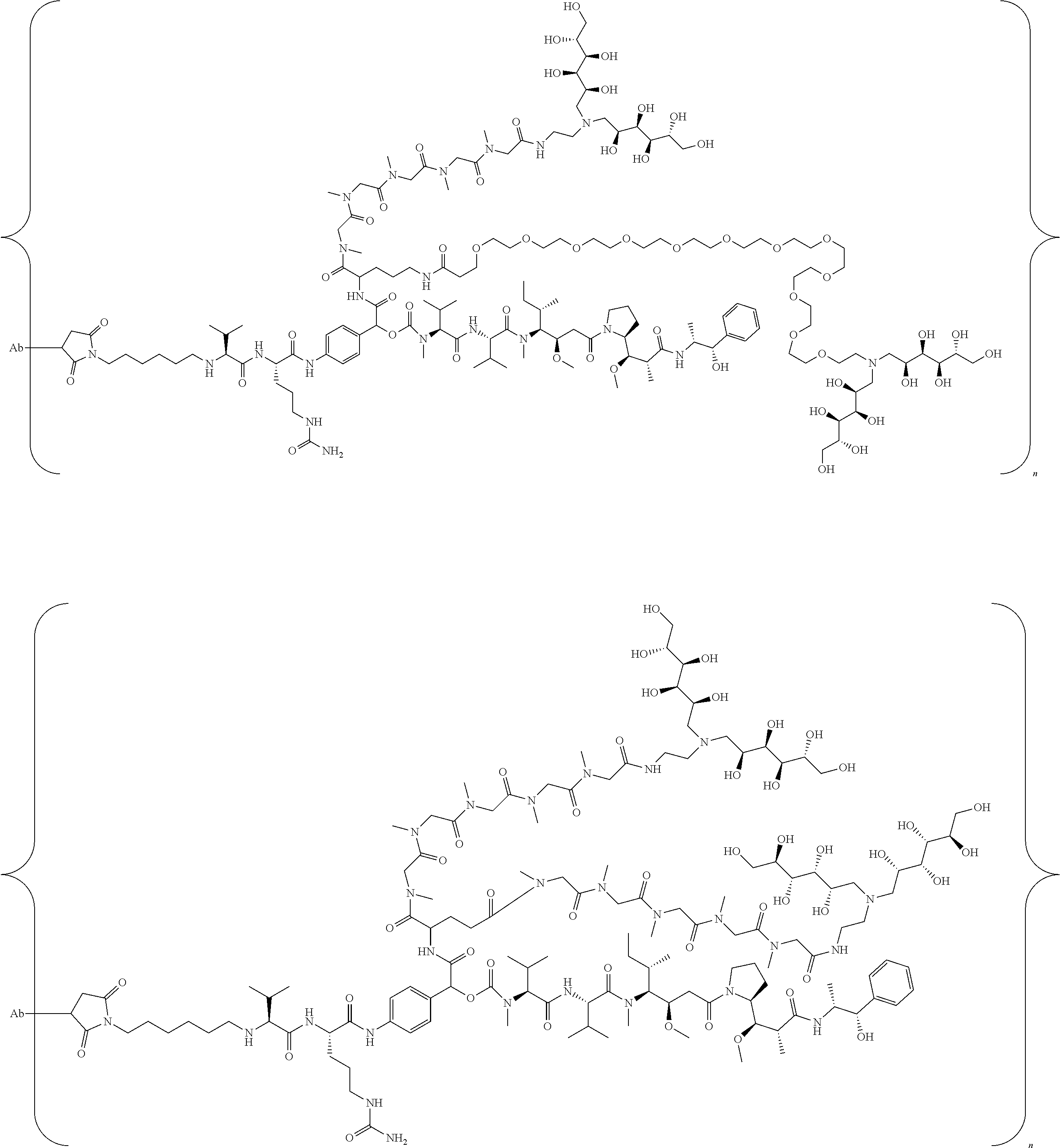 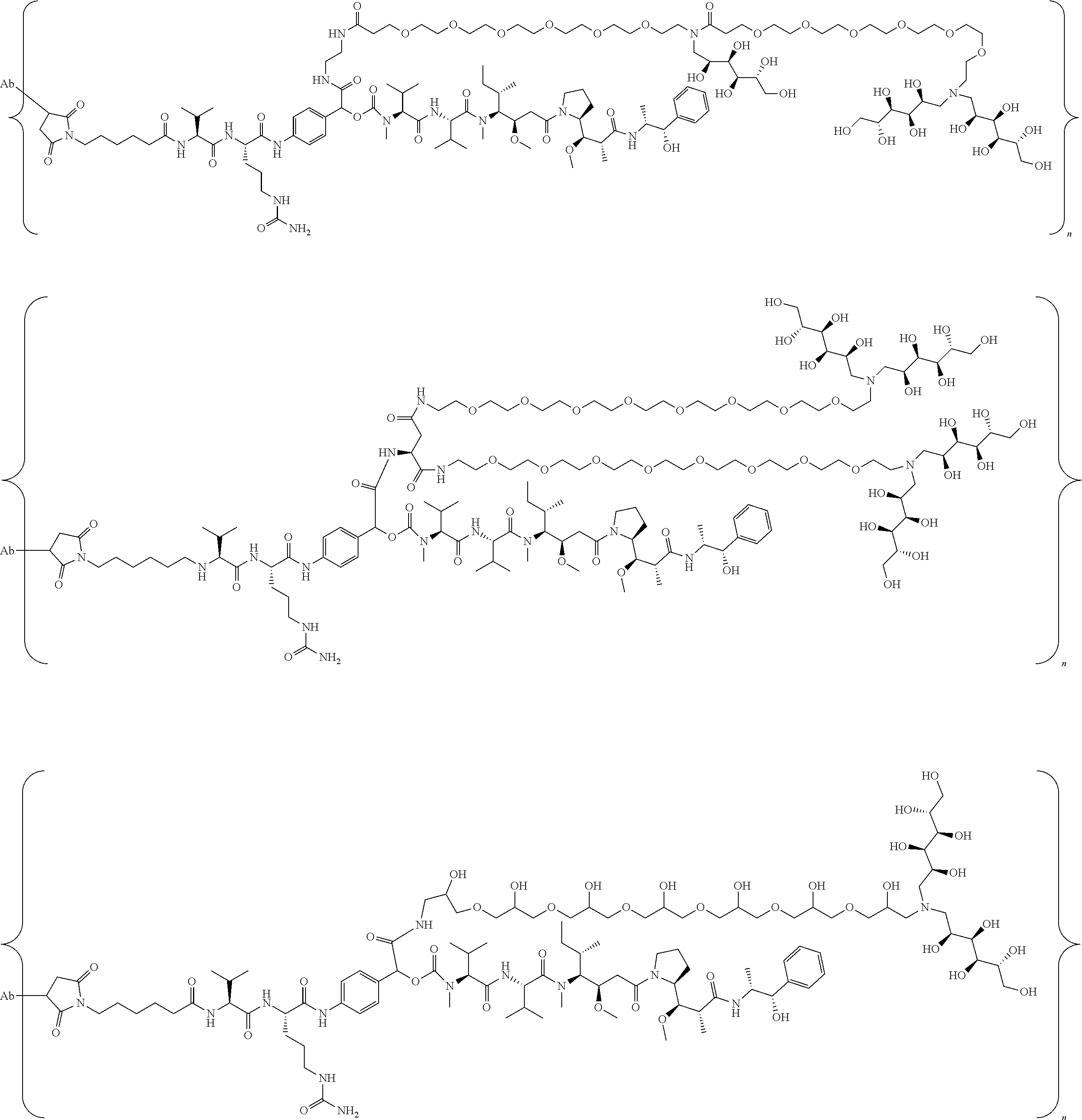 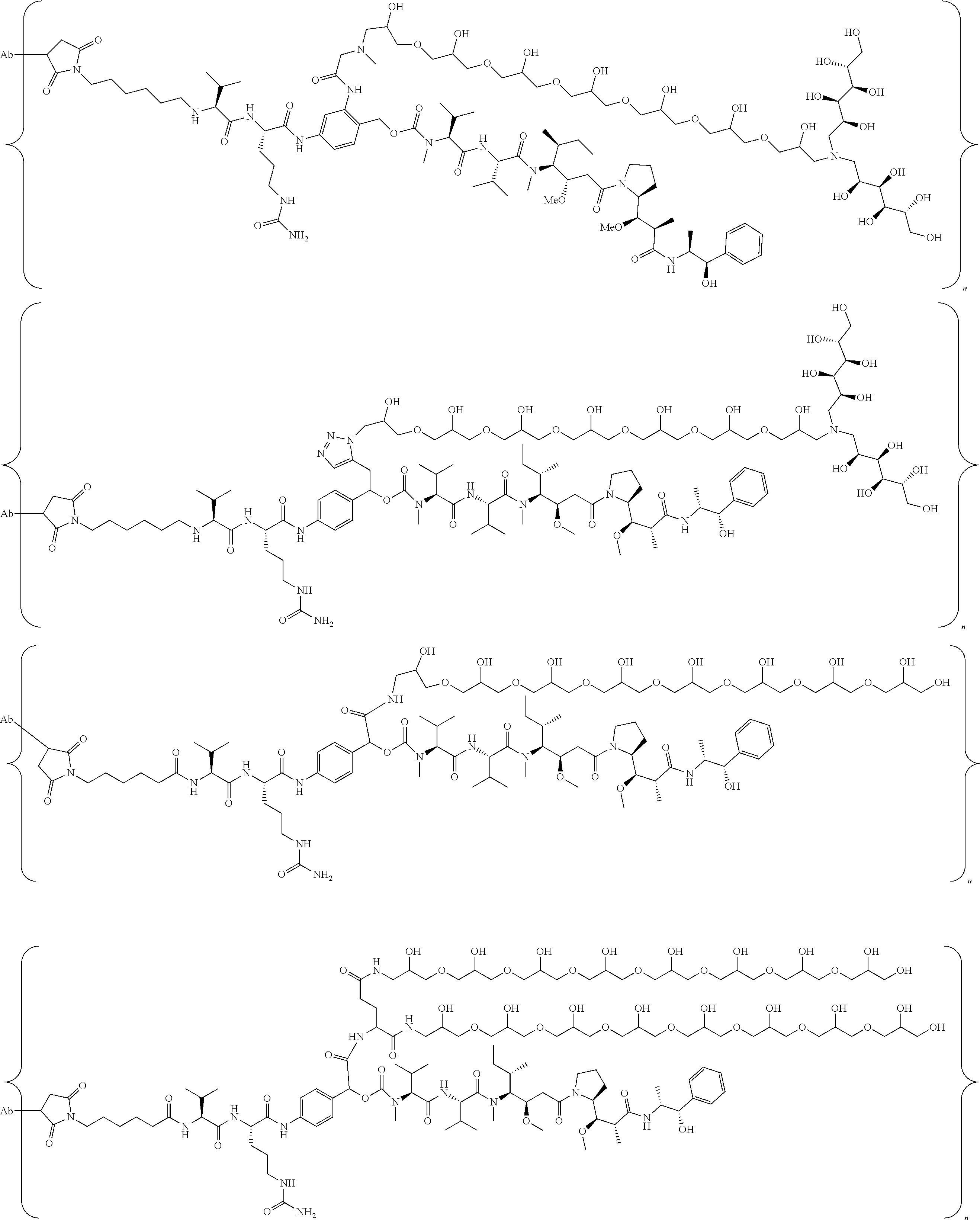 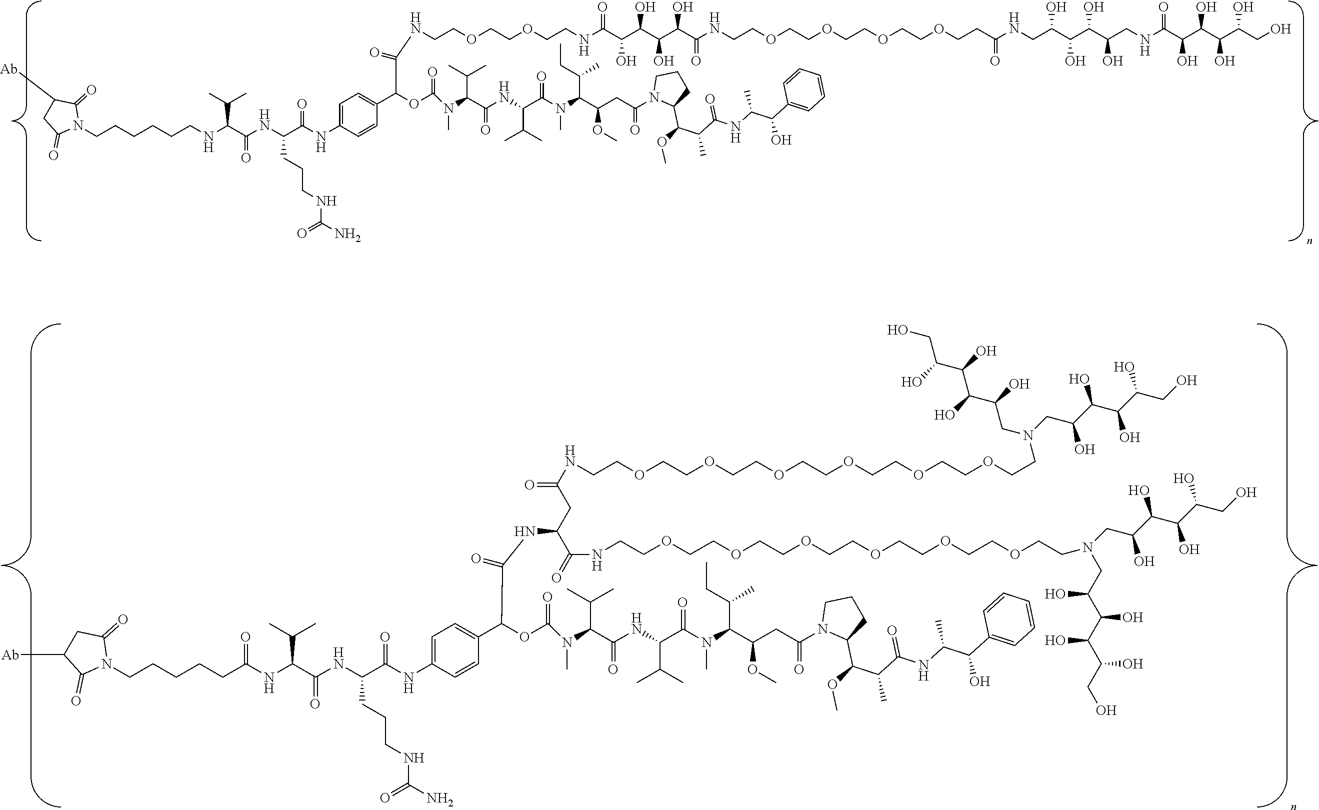 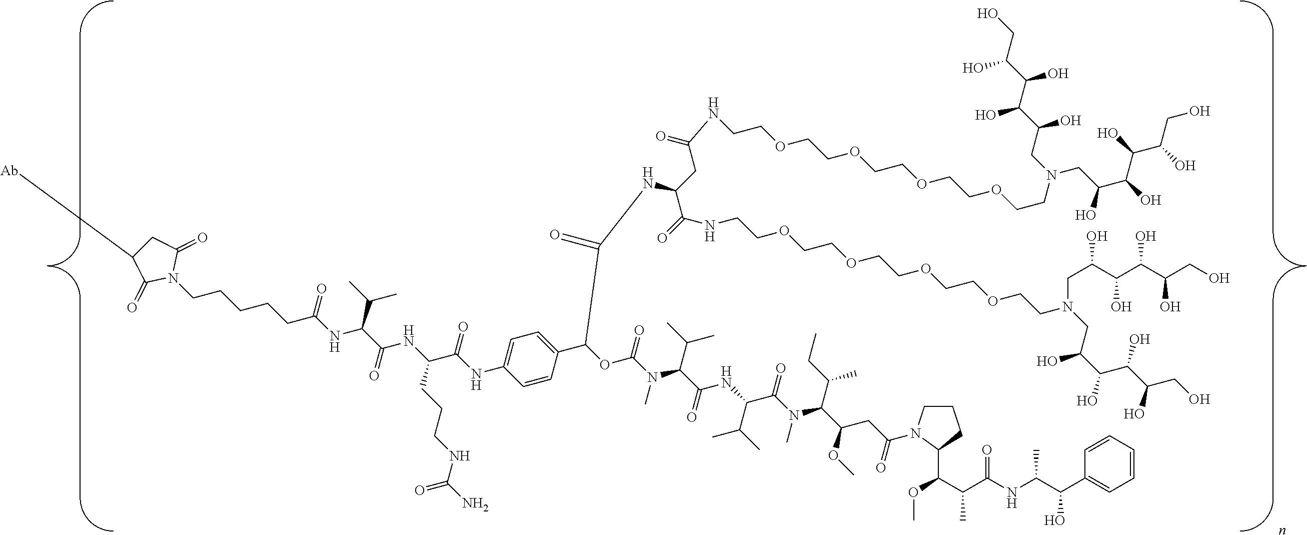 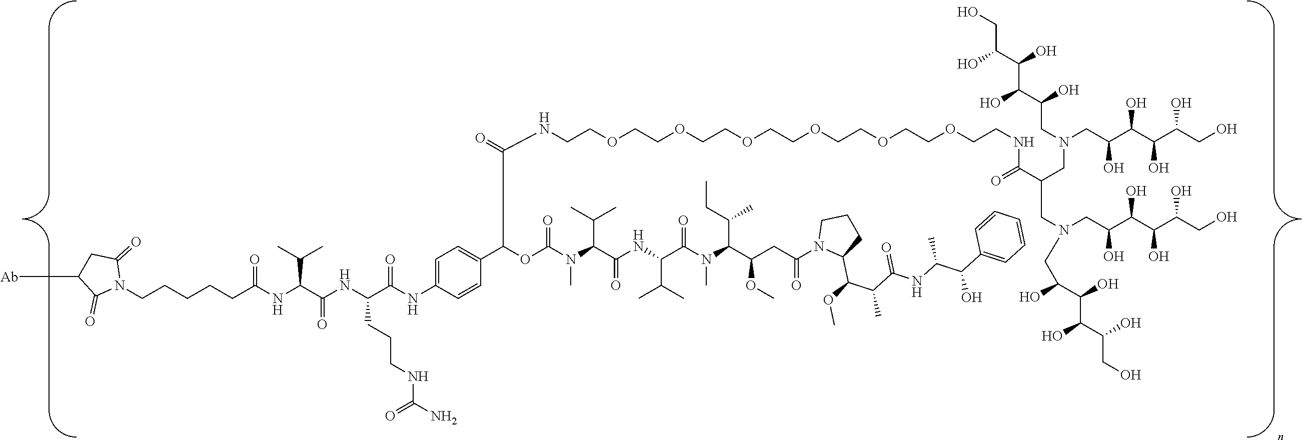 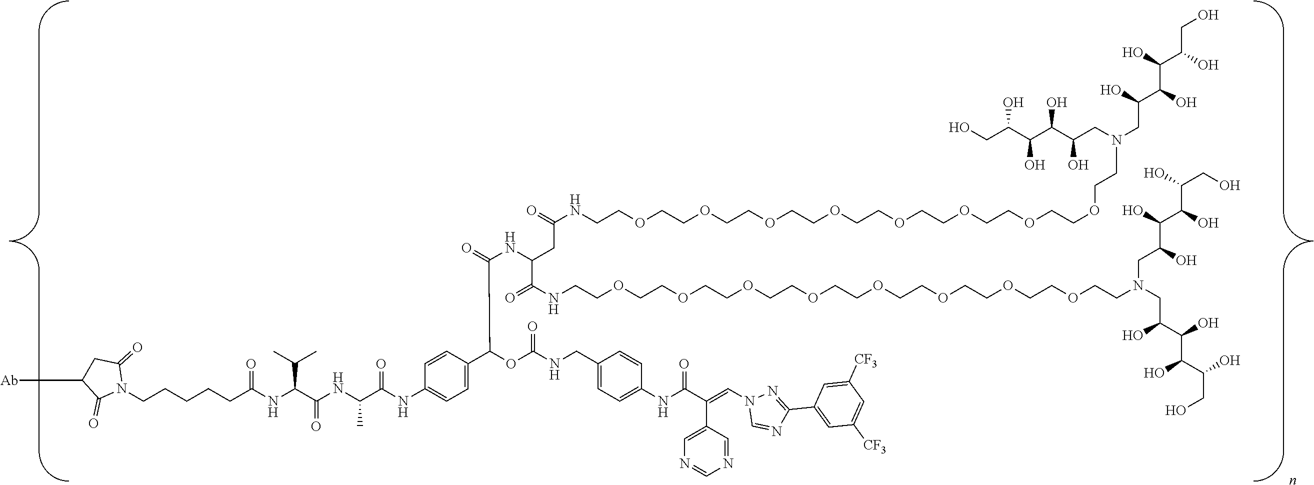 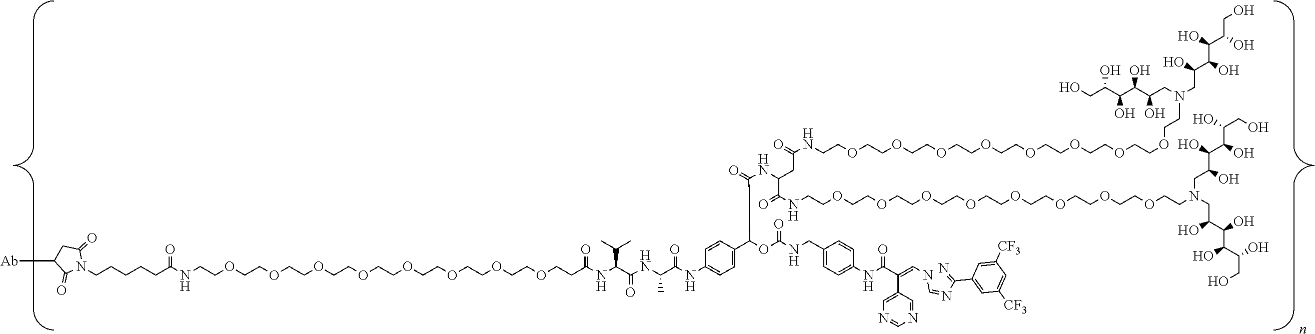 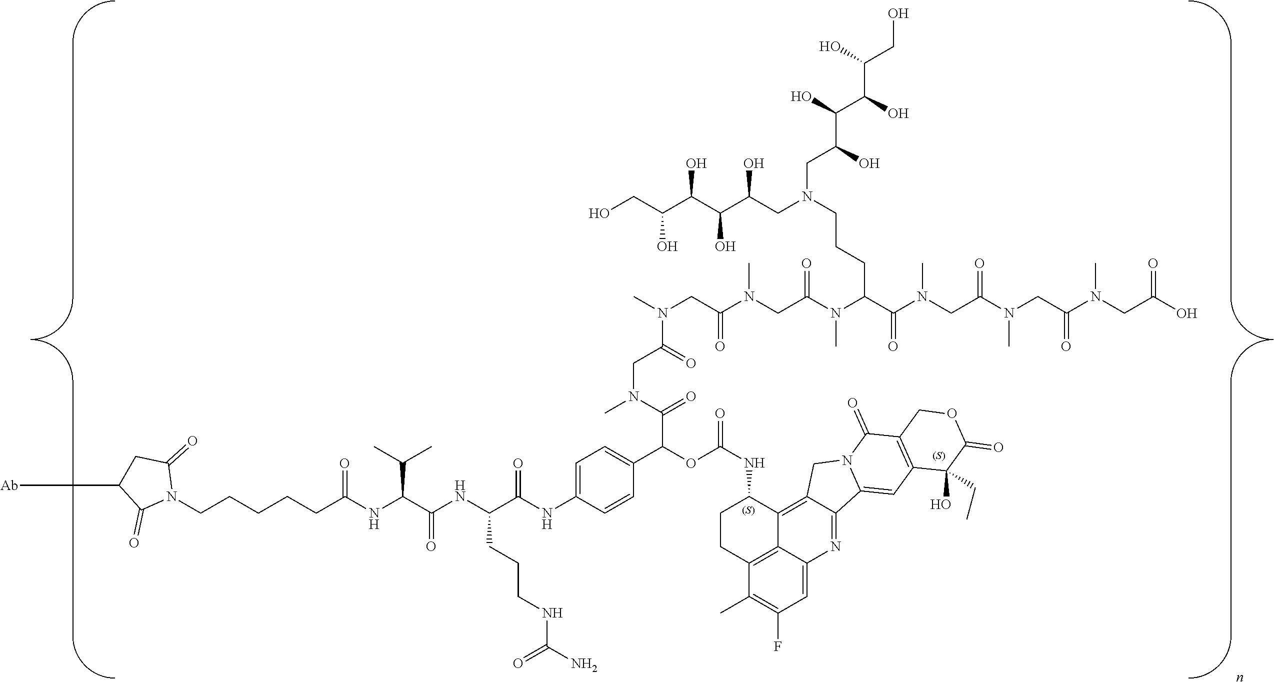 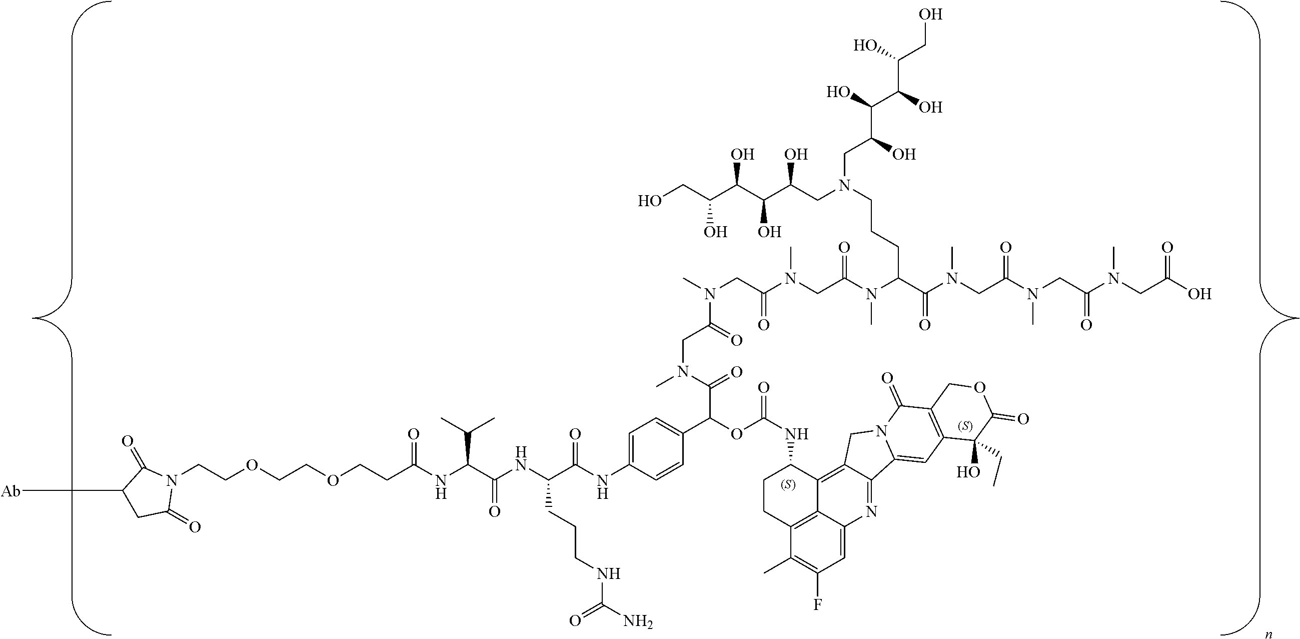 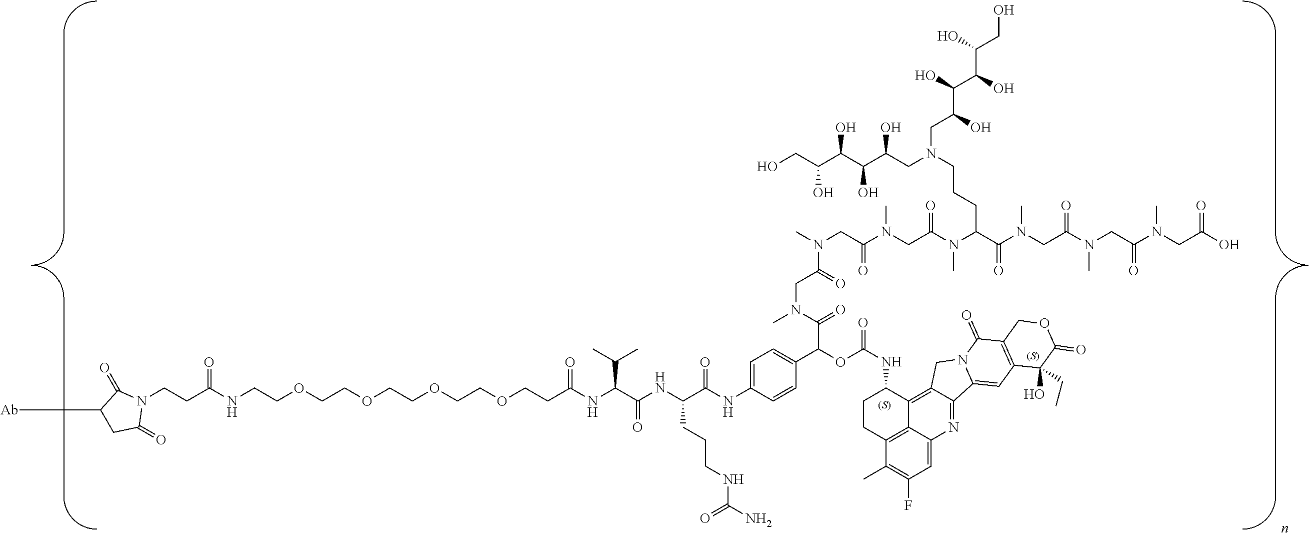 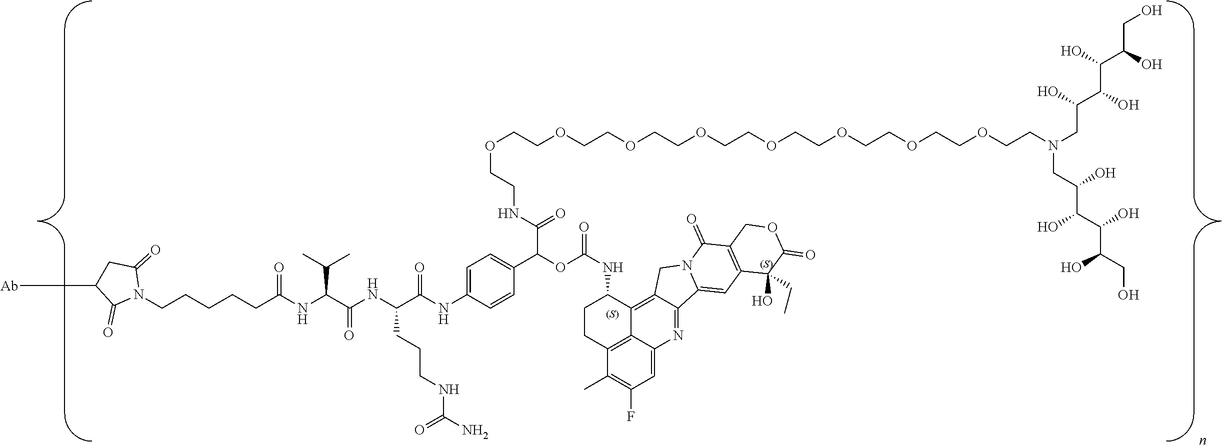 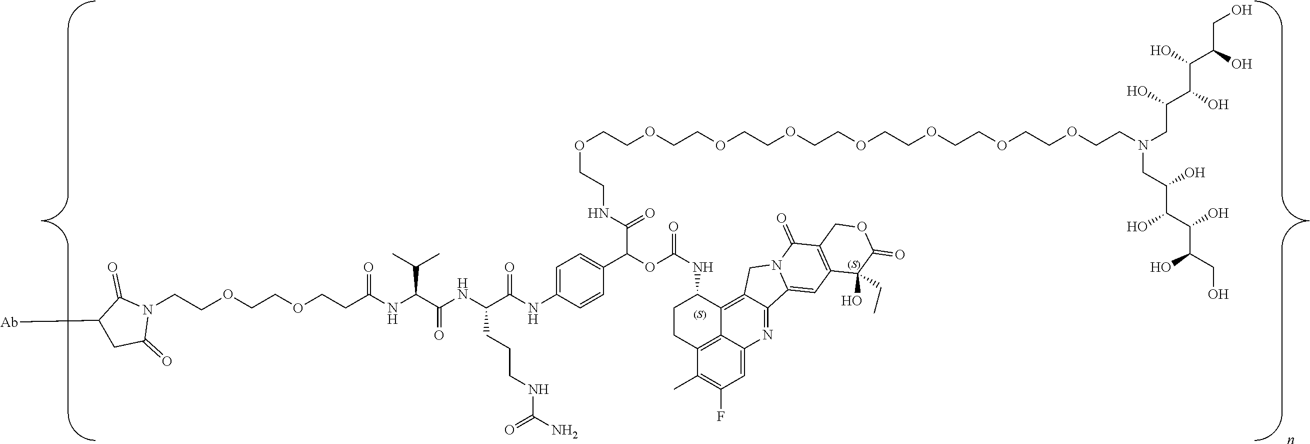 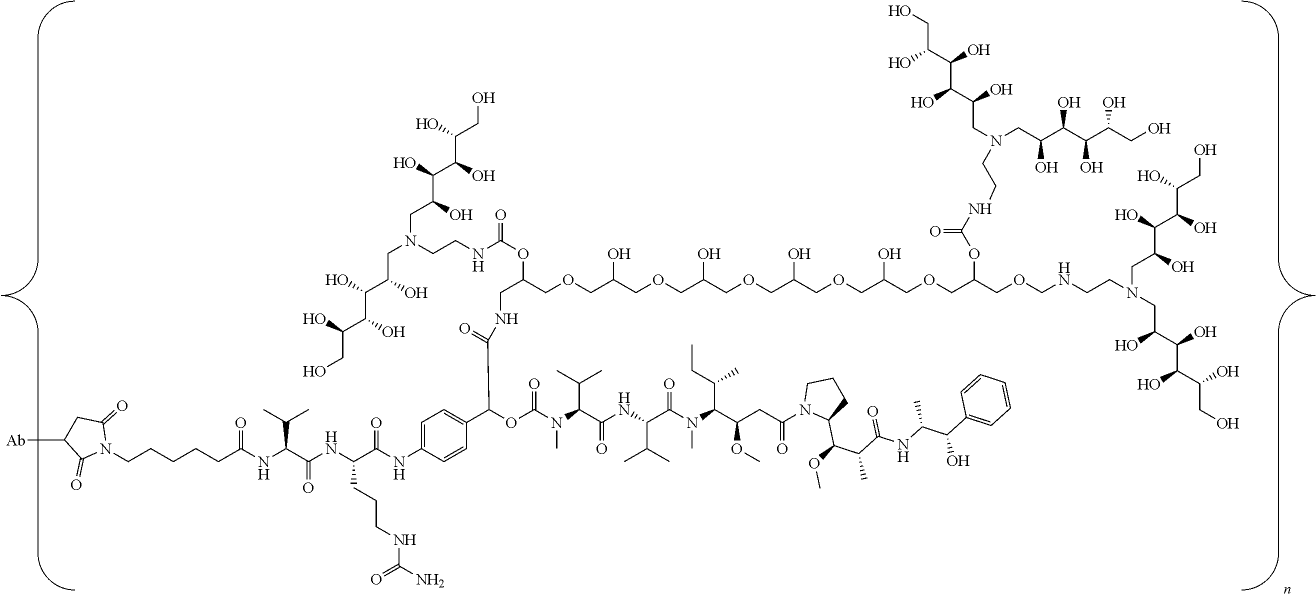 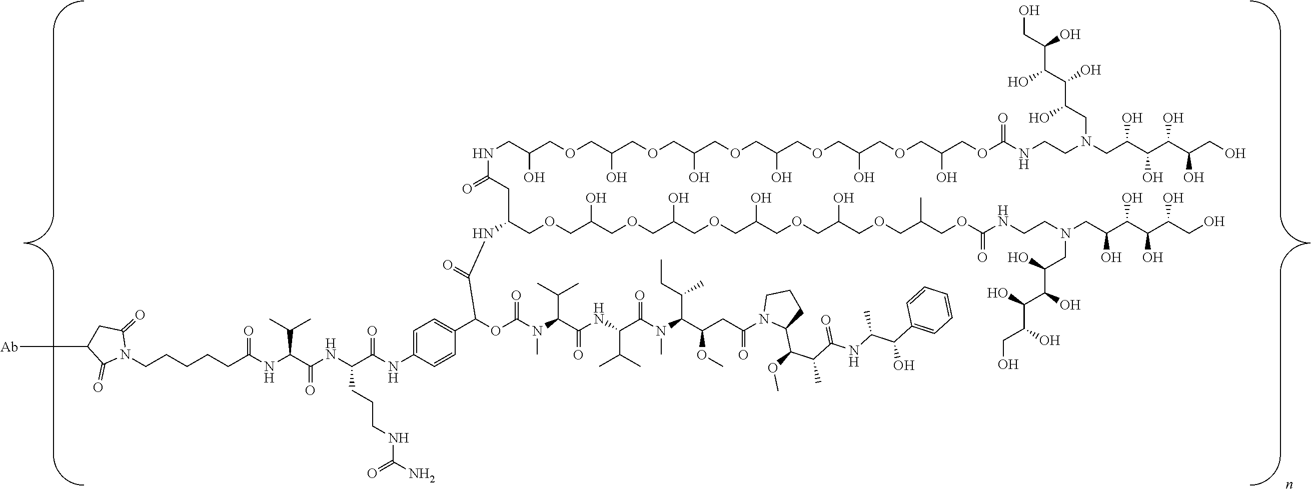 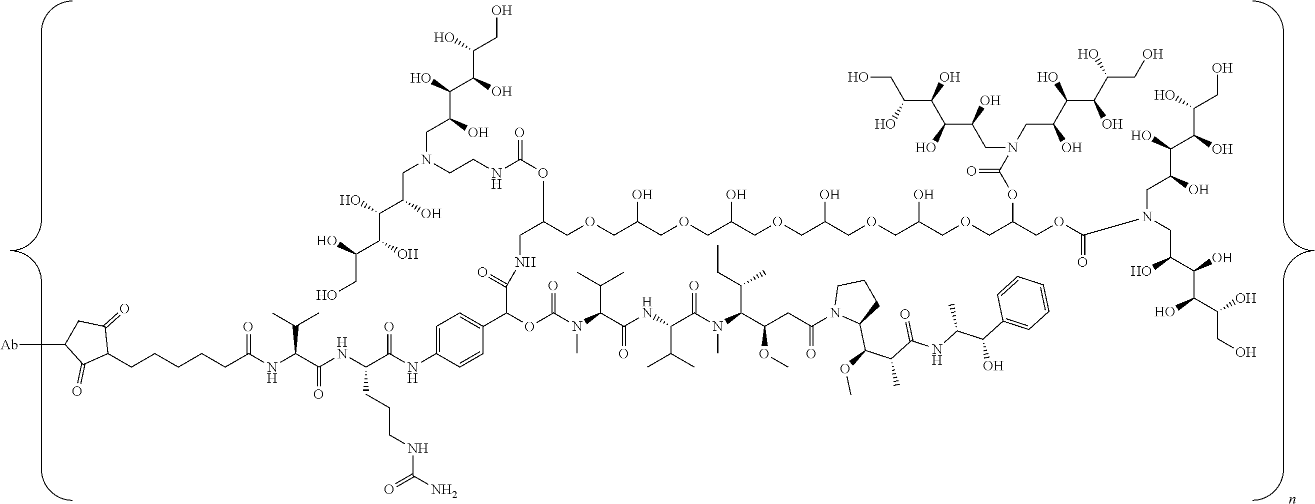 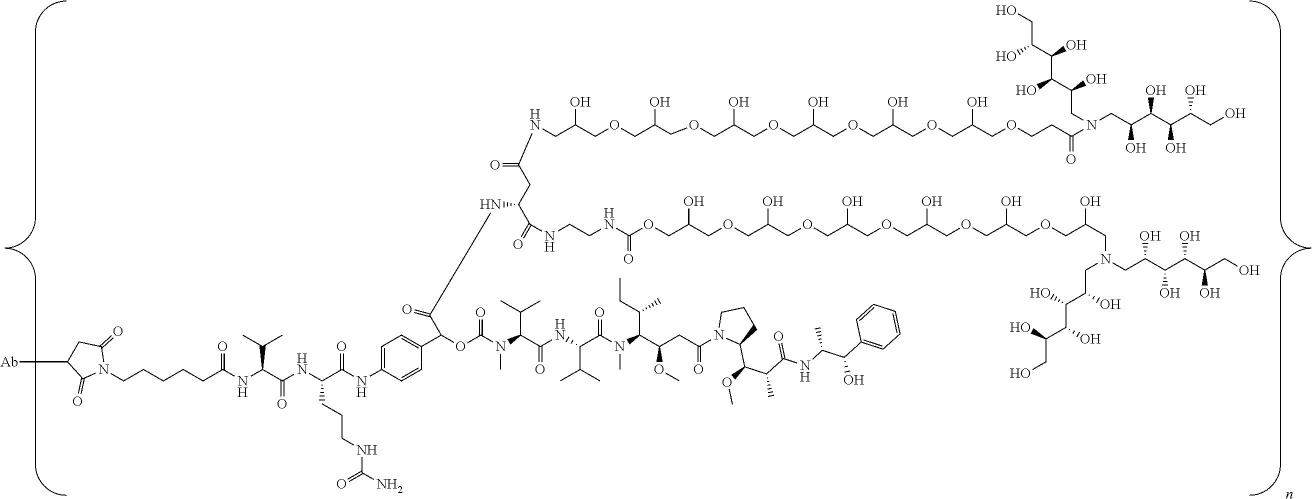 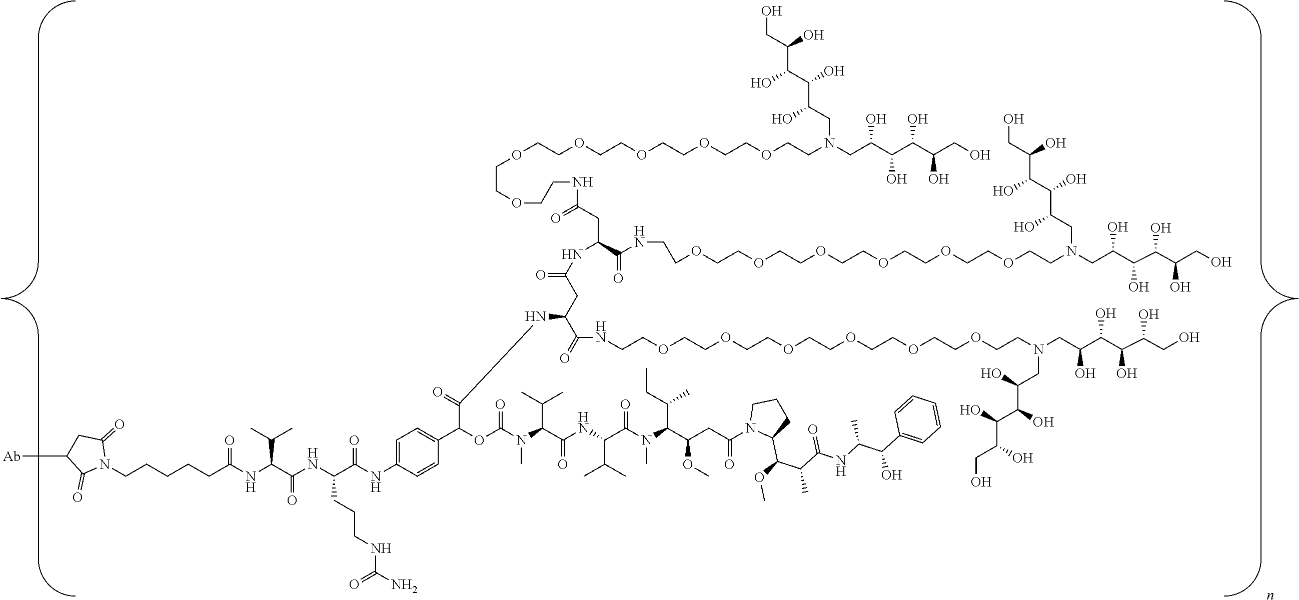 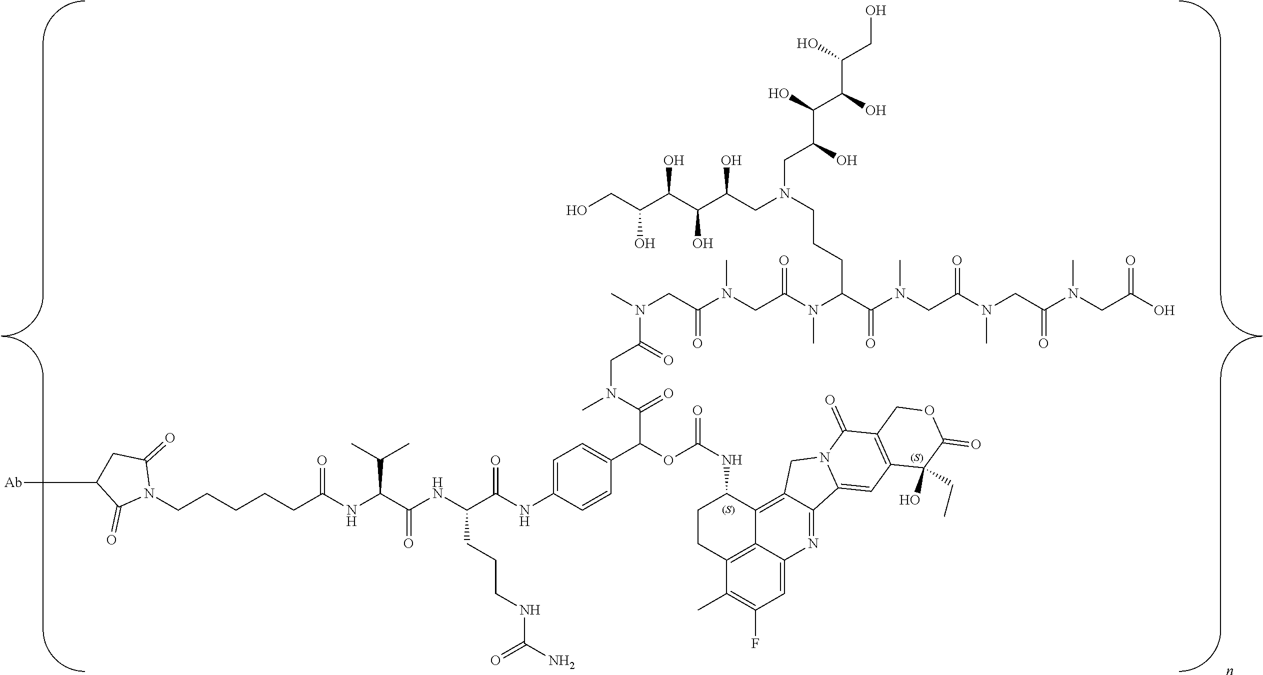 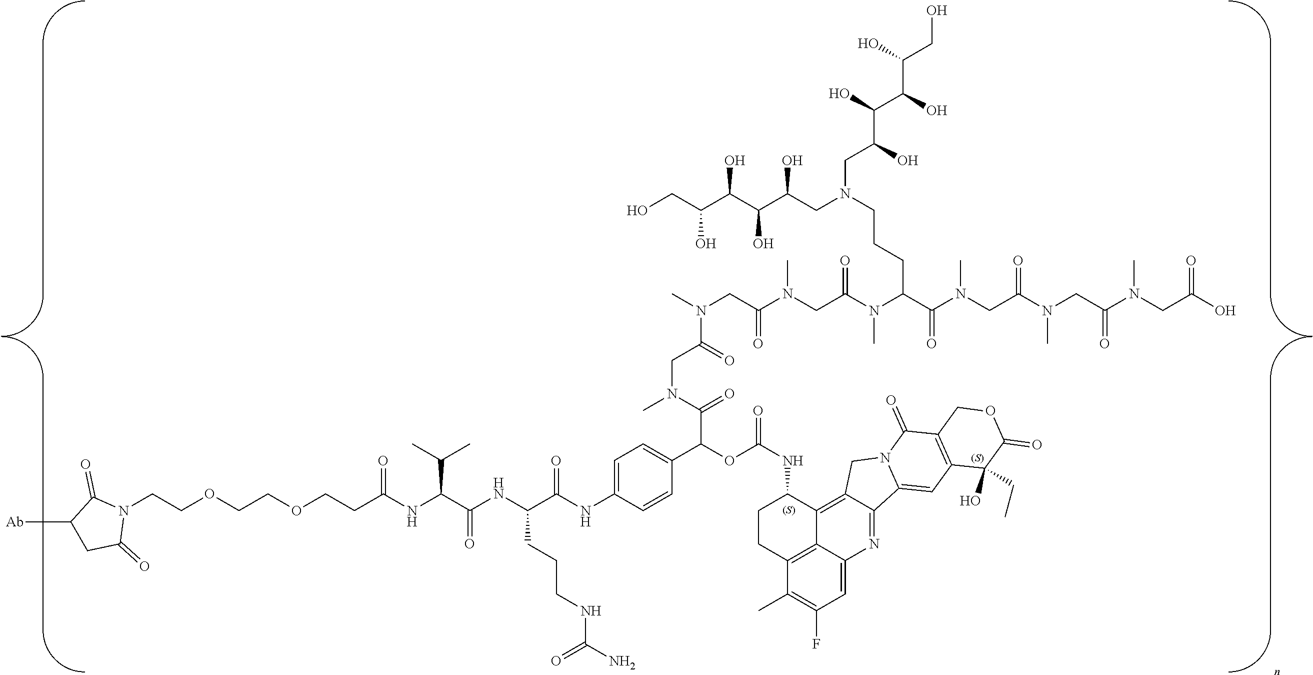 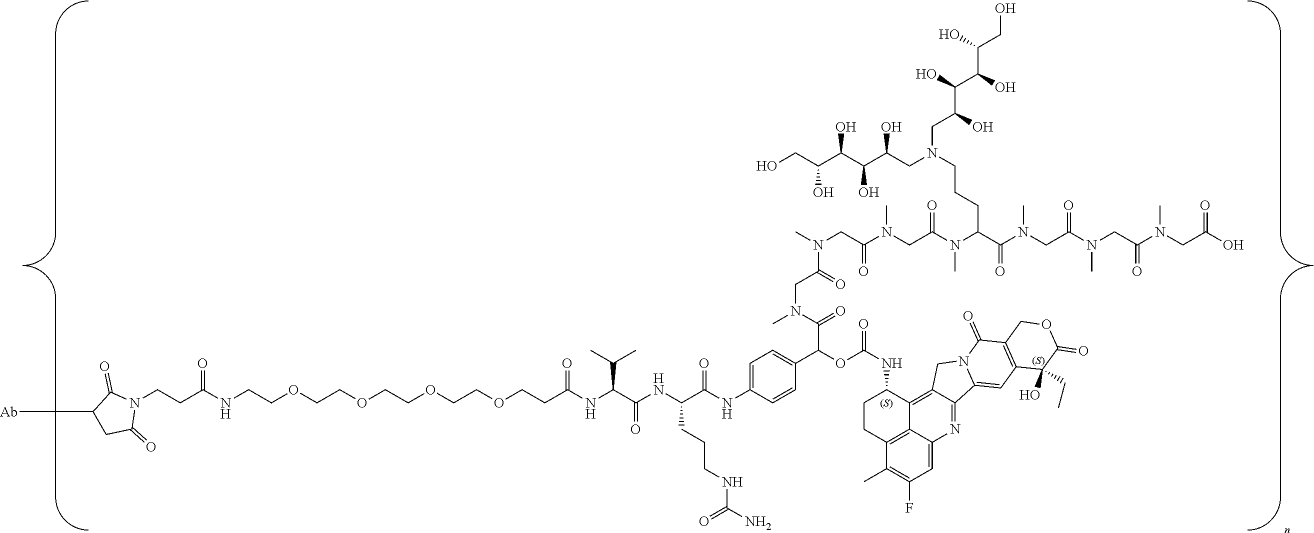 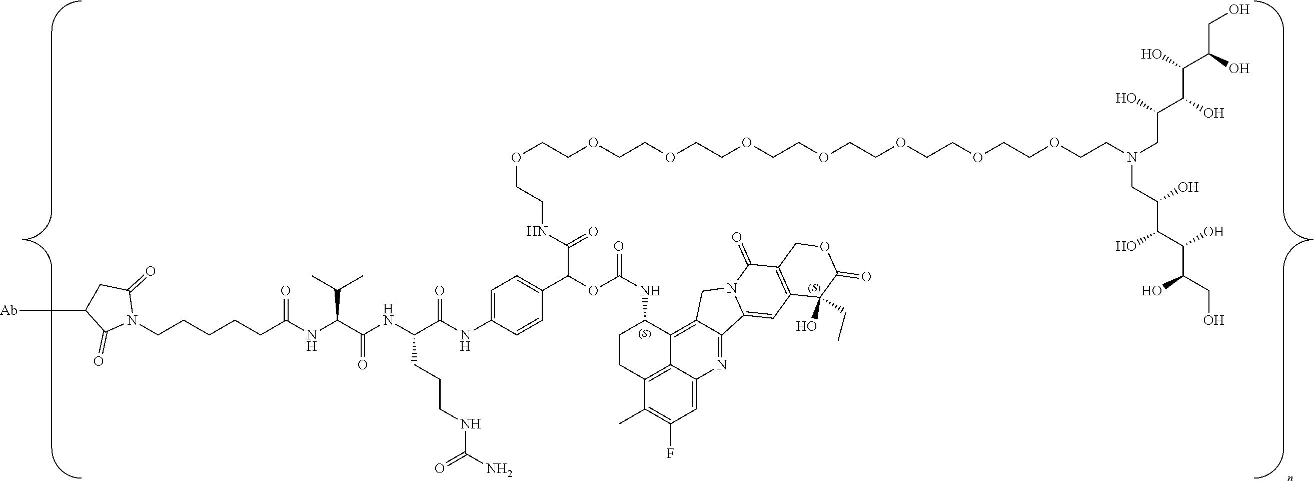 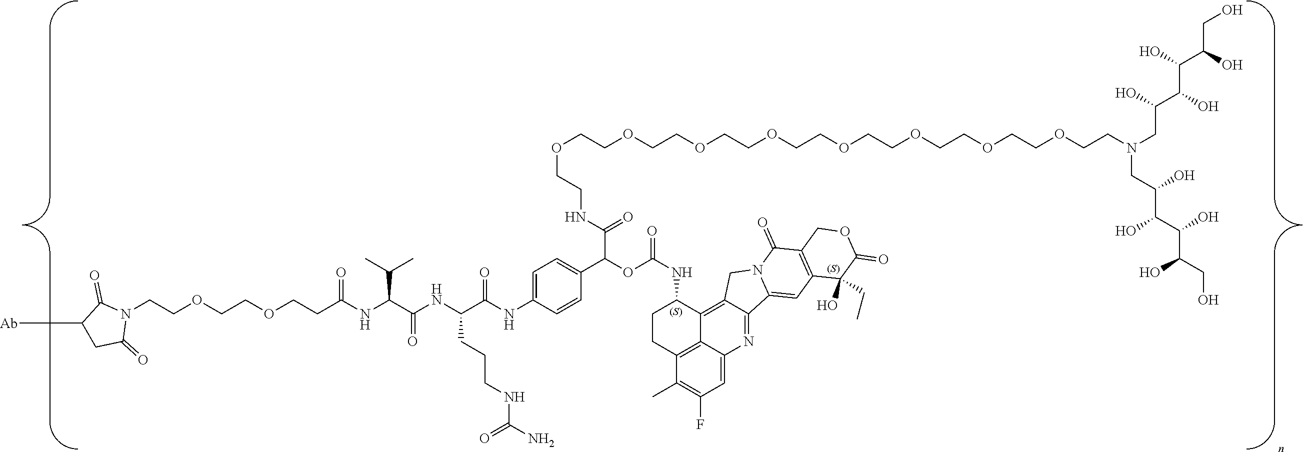 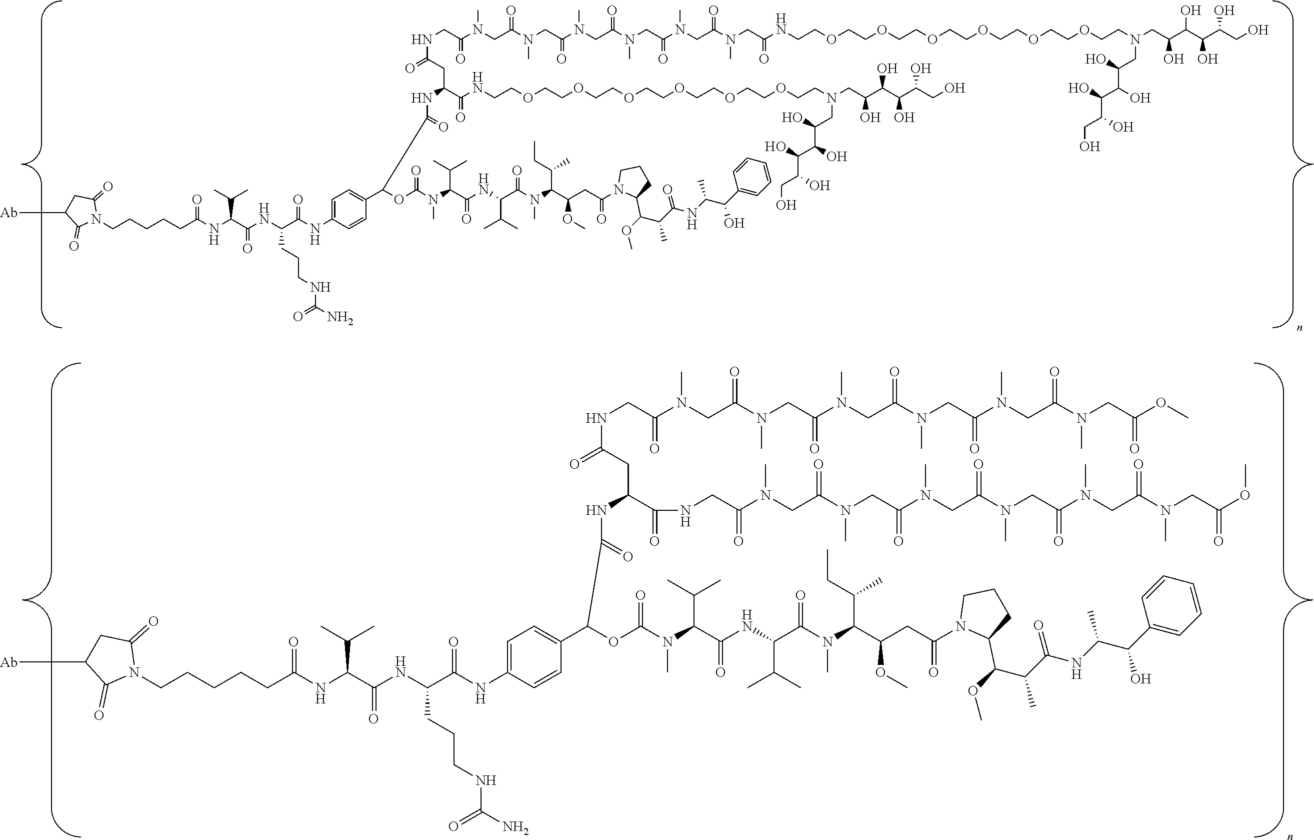 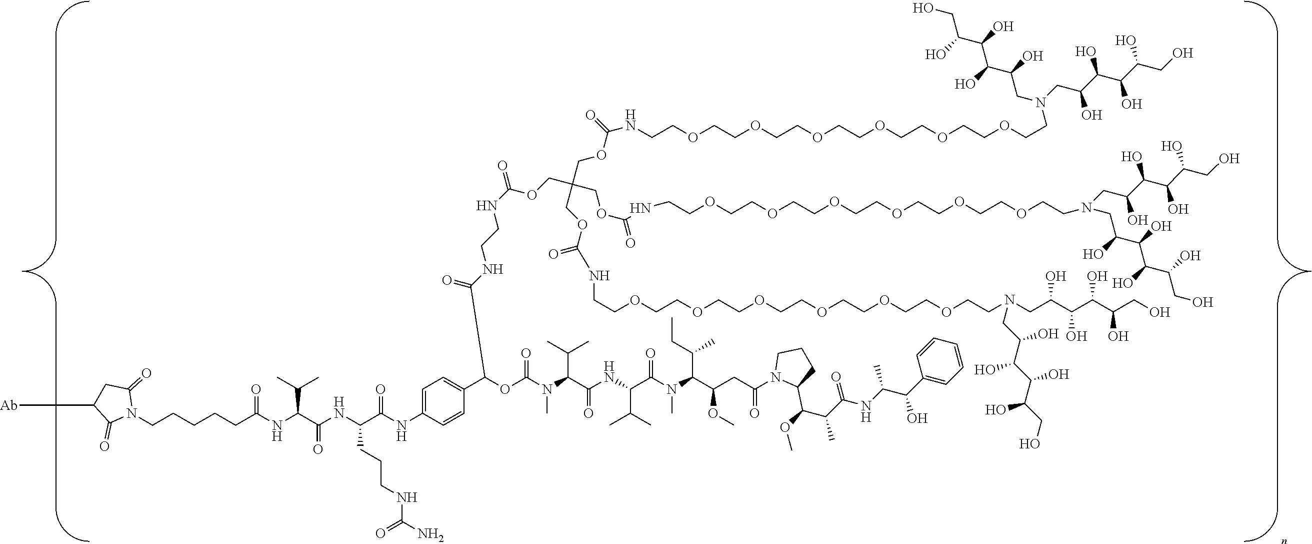 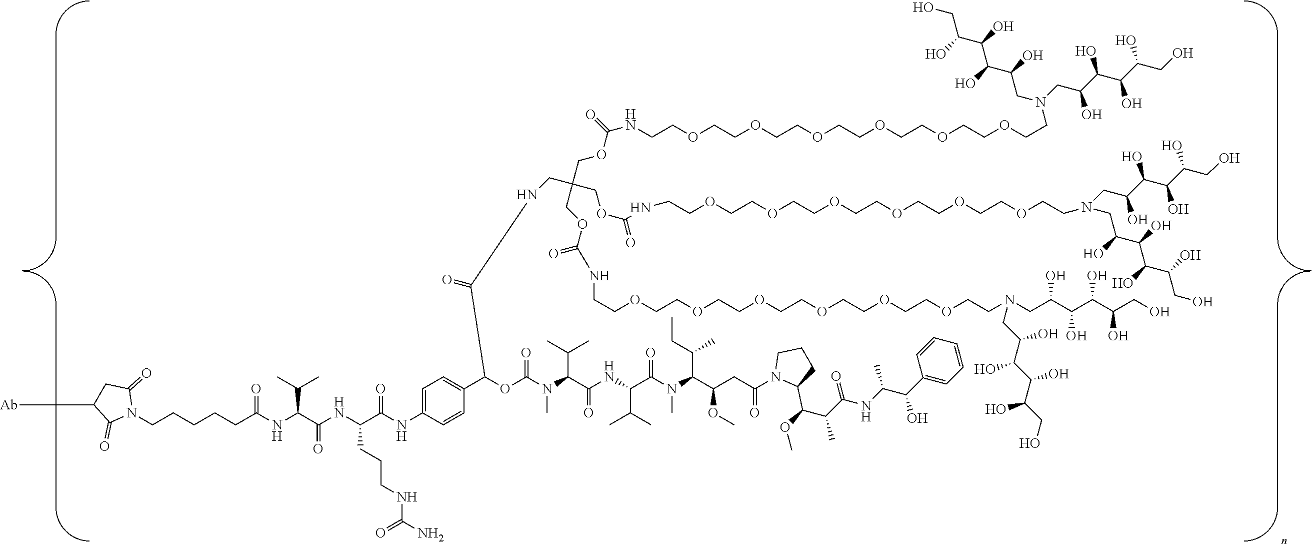 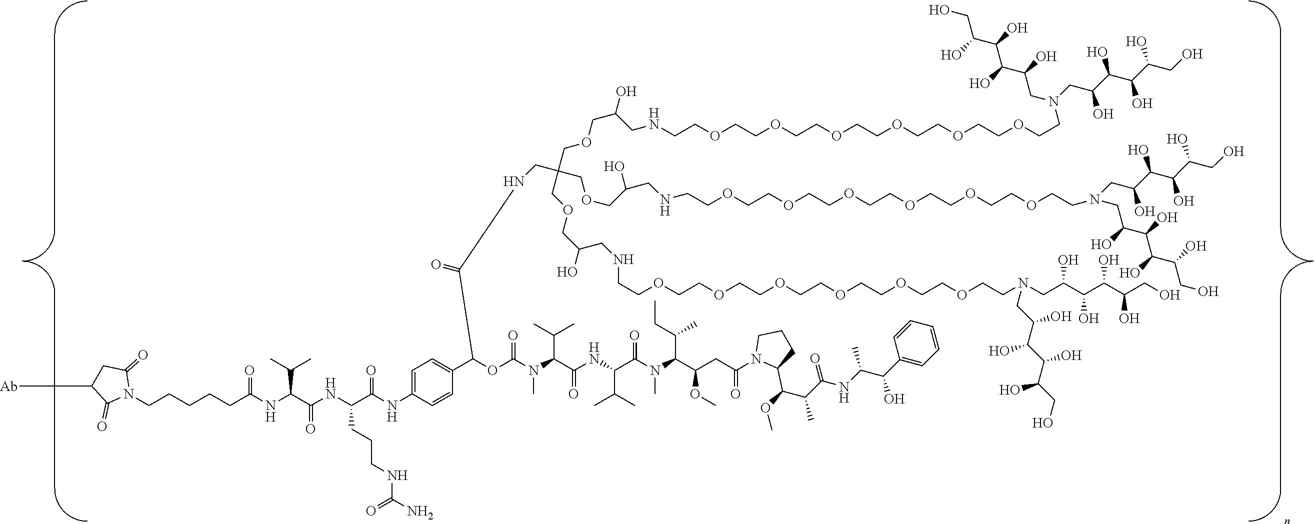 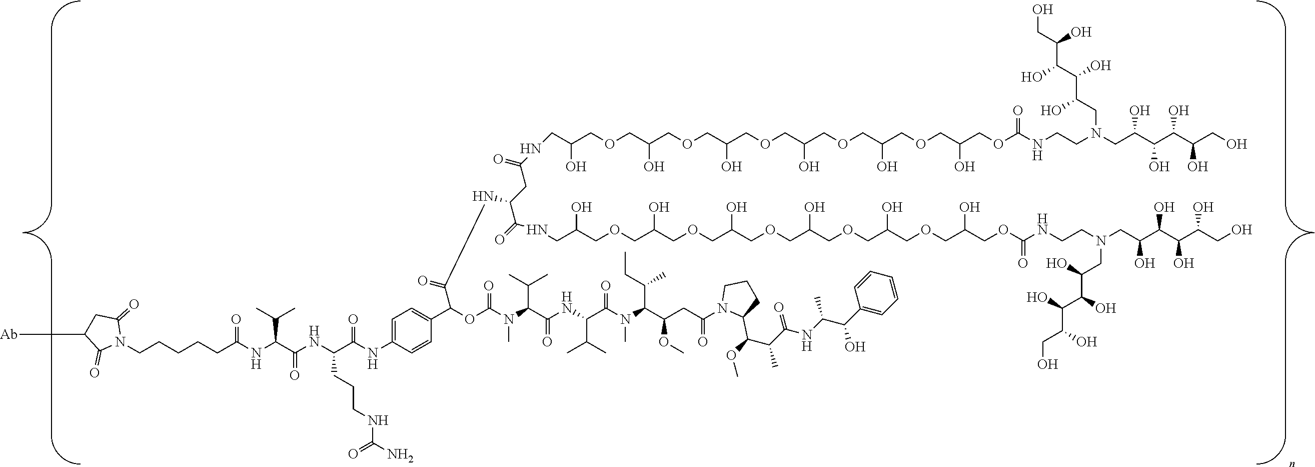 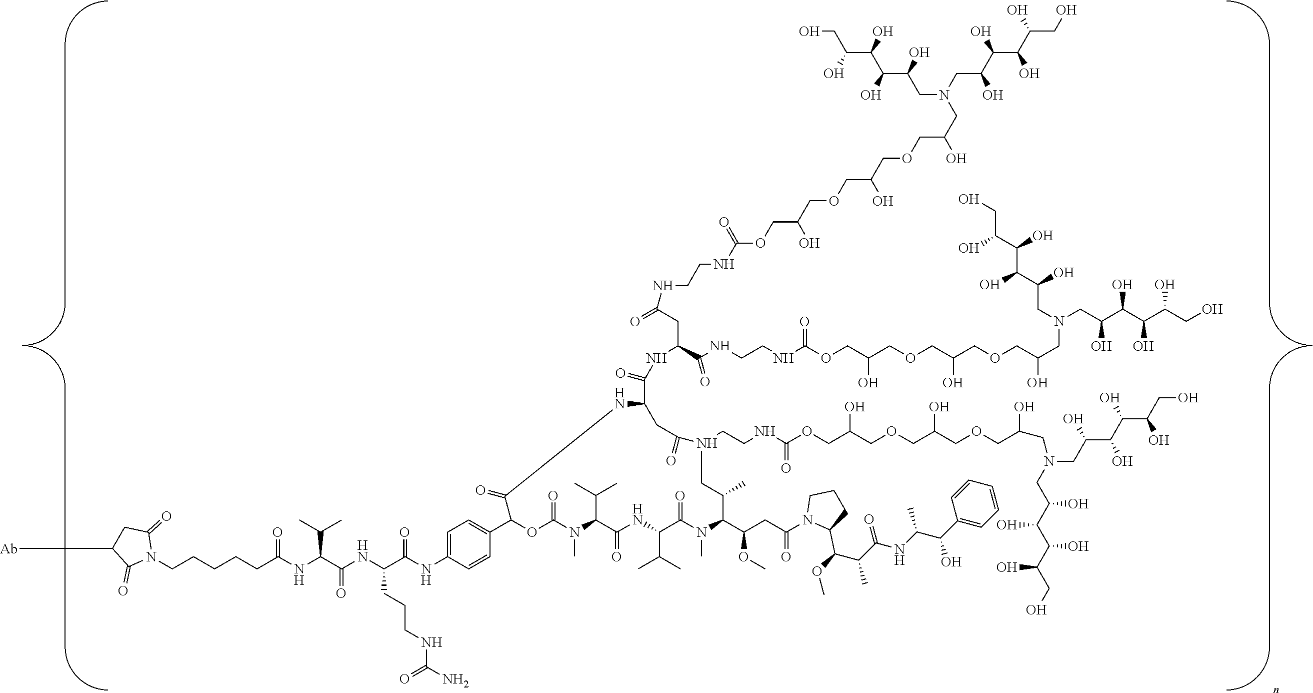 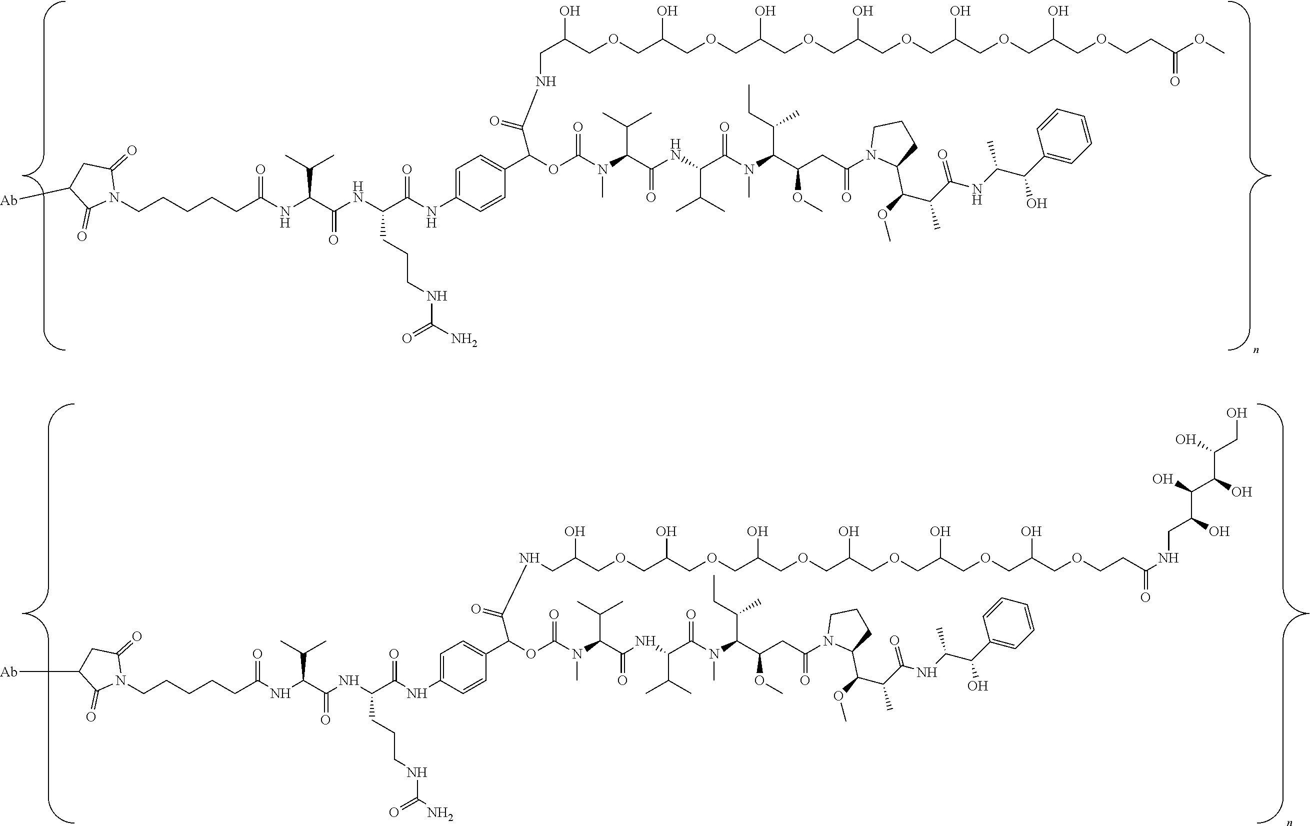 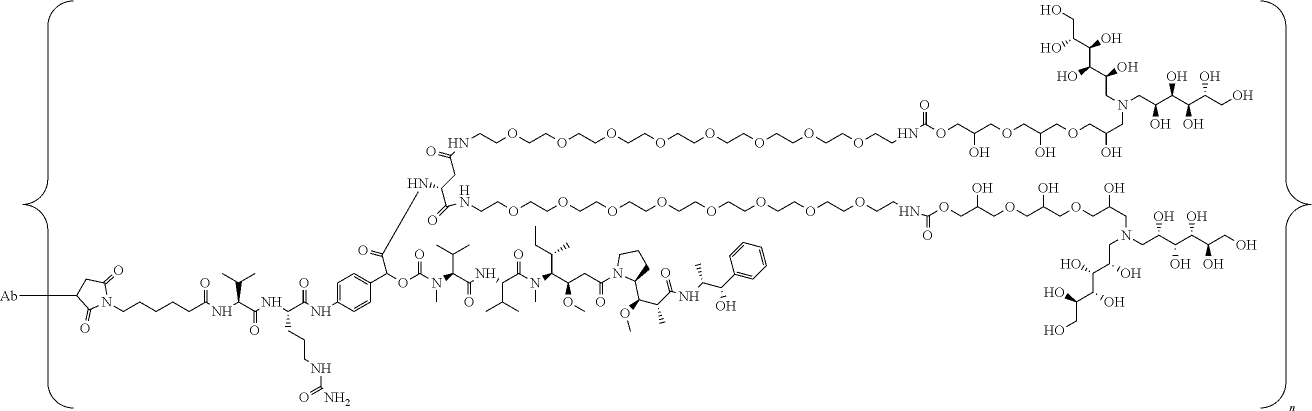 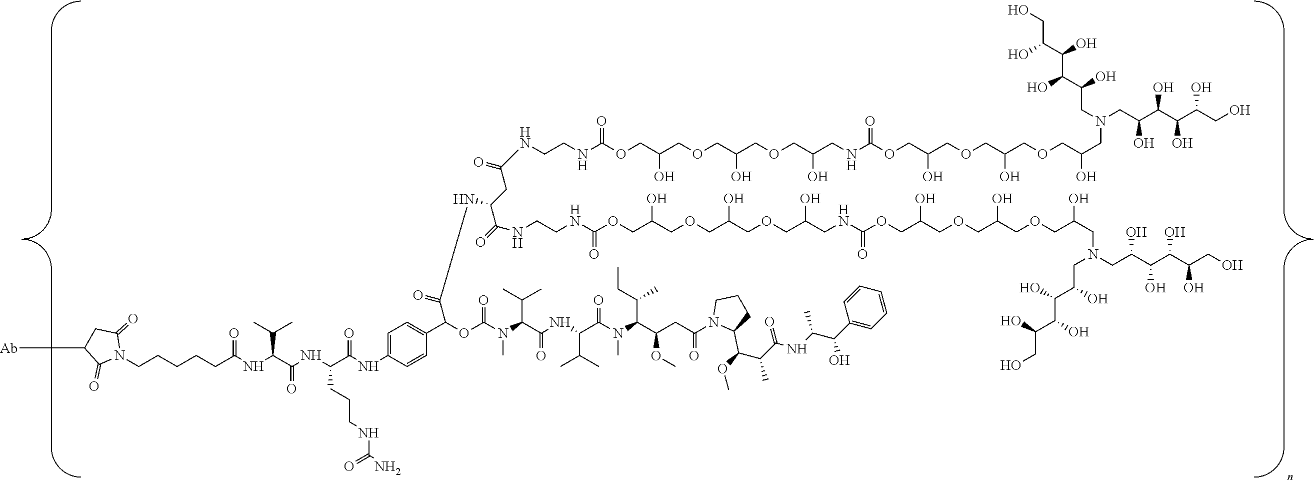 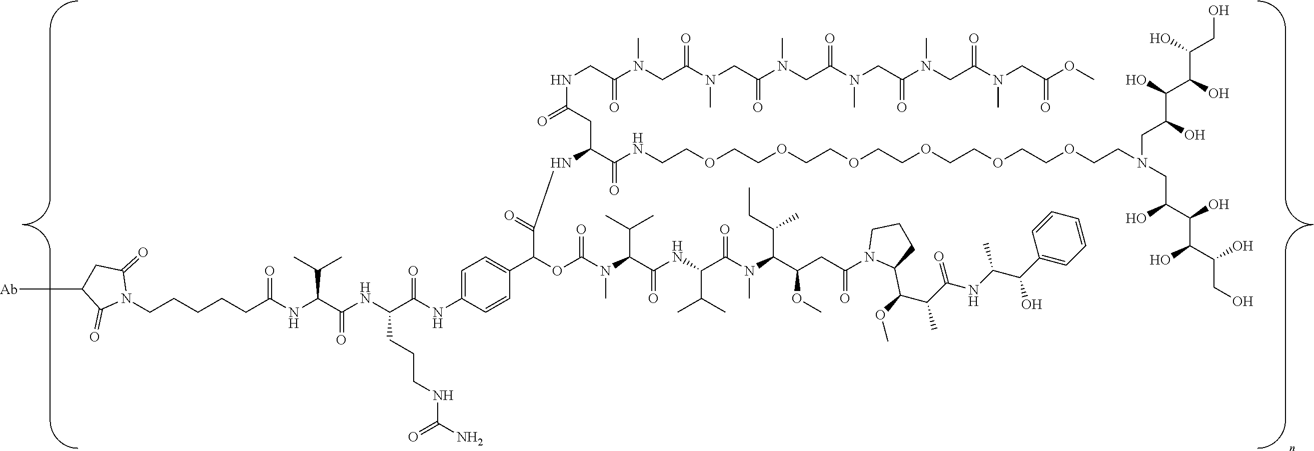 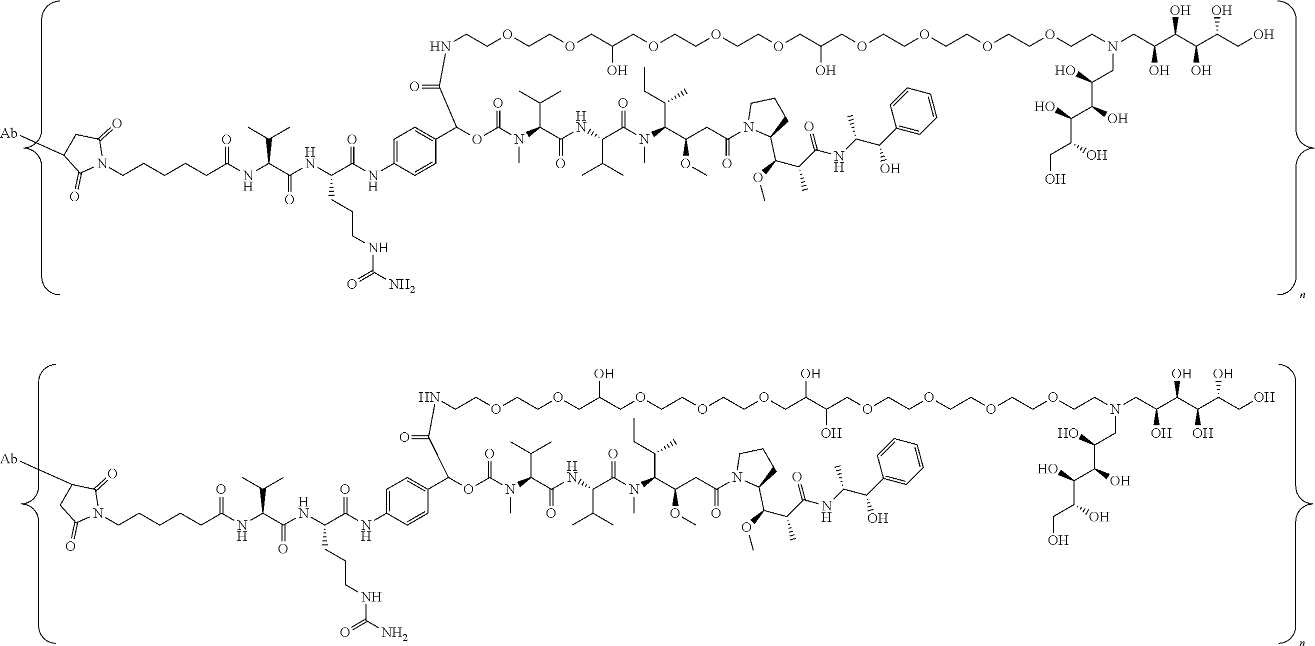 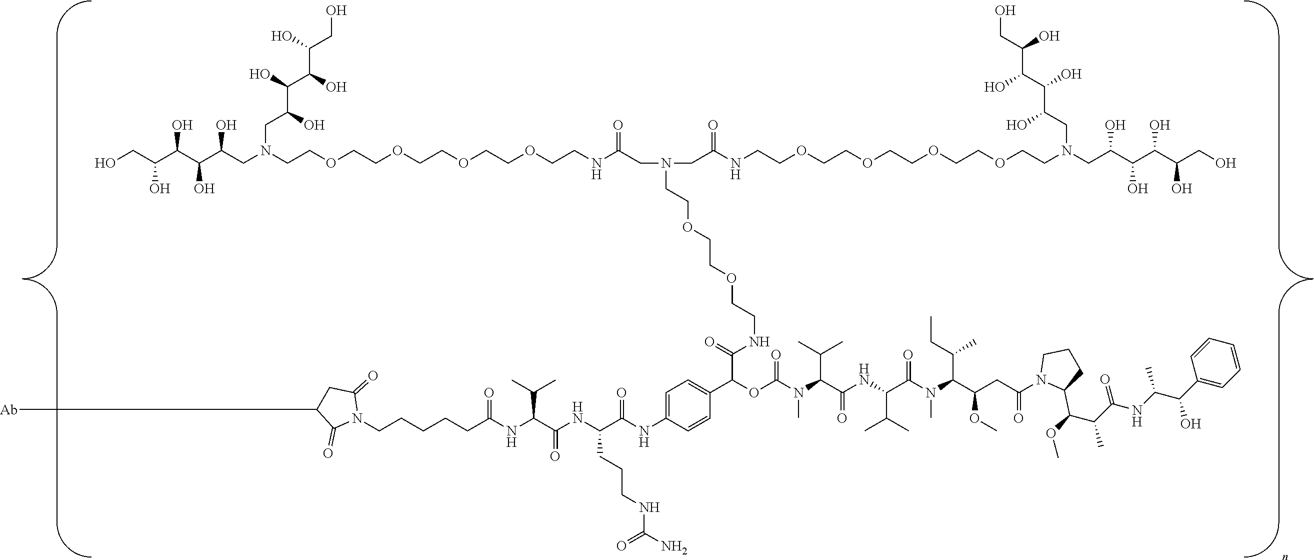 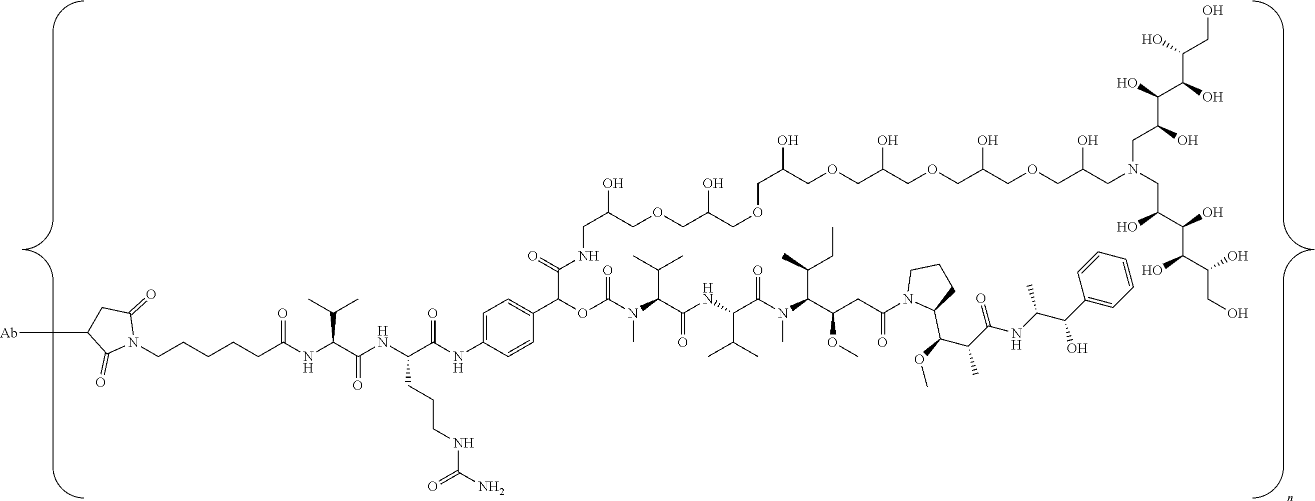 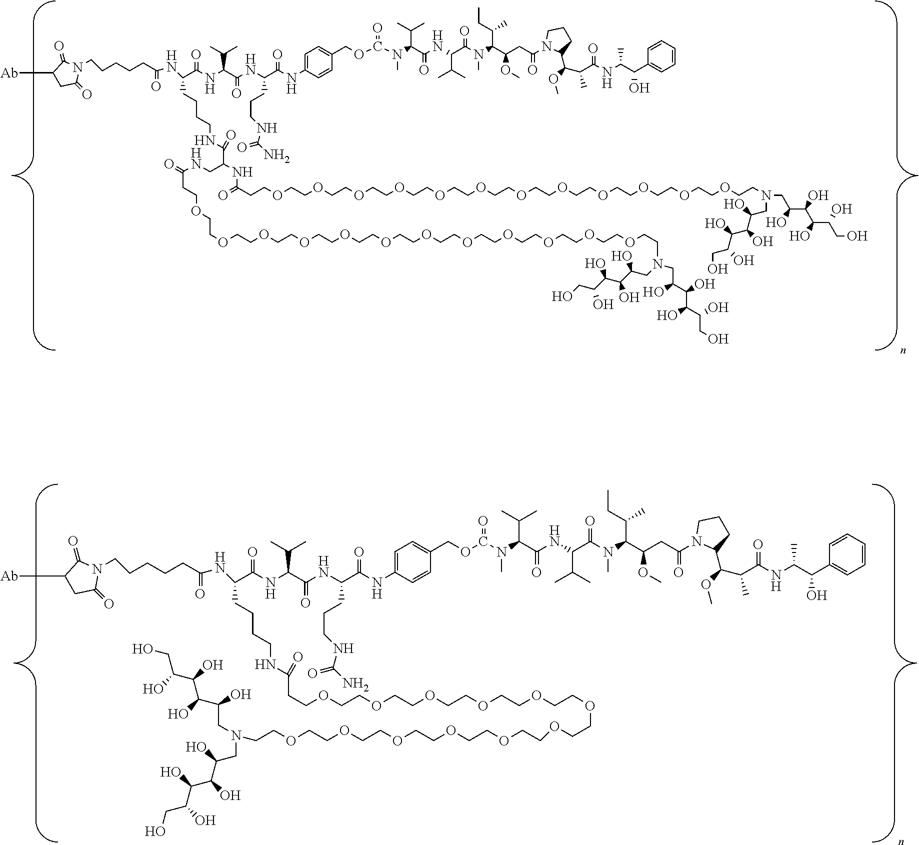 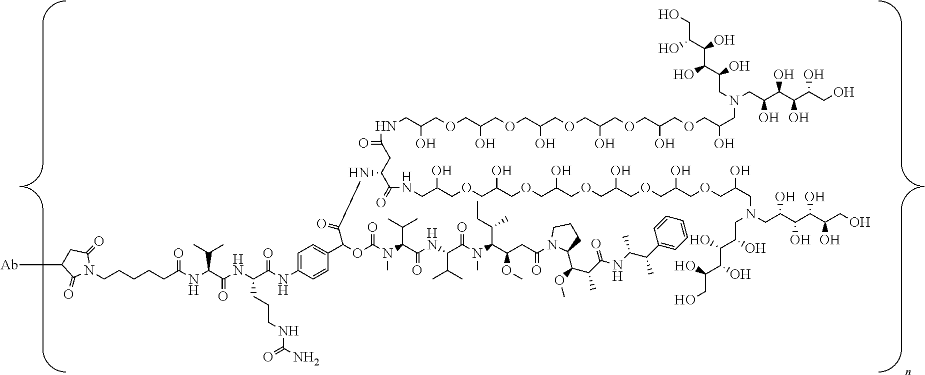
wherein Ab is the binding agent and n is p^{load}.

97. The conjugate of claim 26, wherein the conjugate has the following structure: 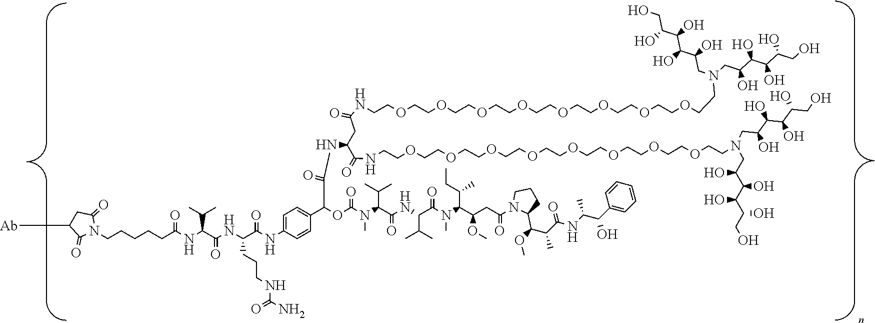
and wherein Ab is the binding agent and n is p^{load} wherein p^{load} is from about 1 to about 8, about 2, about 4, about 6, about 8, about 10, about 12, about 14, about 16, about 3 to about 5, about 6 to about 8, or about 8 to about 16.

98. The conjugate of claim 26, wherein the conjugate has the following structure: 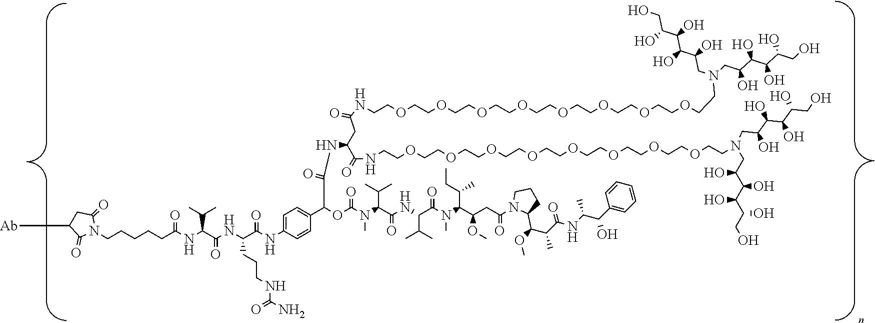
and wherein Ab is the binding agent of claim 4 and n is p^{load}, wherein p^{load} is from about 1 to about 8, about 2, about 4, about 6, about 8, about 10, about 12, about 14, about 16, about 3 to about 5, about 6 to about 8, or about 8 to about 16.

99. The conjugate of claim 26, wherein the conjugate has the following structure: 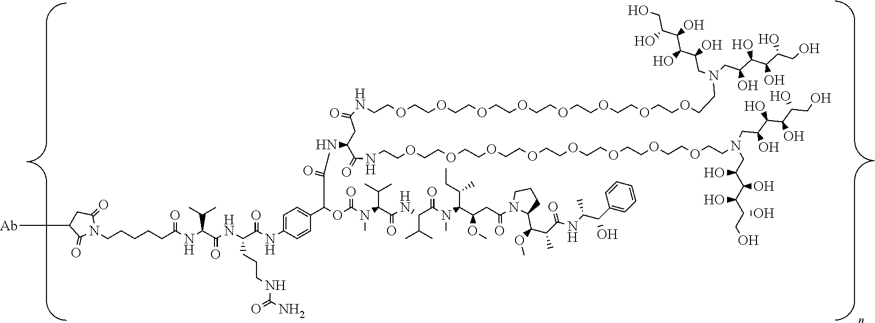
and wherein Ab is the binding agent of claim 21 and n is p^{load}, wherein p^{load} is 8.

100. A pharmaceutical composition comprising the conjugate of claim 26 and a pharmaceutically acceptable carrier.

101. A method of treating a PTK7+ cancer, comprising administering to a subject in need thereof a therapeutically effective amount of the binding agent of claim 1.

102. The method of claim 101, wherein the PTK7+ cancer is a solid tumor or a hematologic malignancy.

103. The method of claim 102, wherein the PTK7+ cancer is selected from breast cancer (BC), lung cancer (LC), ovarian cancer (OVCA), esophageal cancer (EsC), gastric cancer (GC), bladder cancer (BLC), endometrial cancer (EC), head and neck cancer (HNC), cervical cancer, pharynx cancer, stomach cancer, myeloma, uterine cancer, colon cancer, hepatocellular cancer, and colorectal cancer.

104.-121. (canceled)

122. The method of claim 101, further comprising administering an immunotherapy to the subject.

123. (canceled)

124. The method of claim 122, wherein the immunotherapy comprises a checkpoint inhibitor and wherein the checkpoint inhibitor is selected from an antibody that specifically binds to human PD-1, human PD-L1, or human CTLA4.

125. The method of claim 124, wherein the checkpoint inhibitor is pembrolizumab, nivolumab, cemiplimab or ipilimumab.

126. The method of claim 122, further comprising administering chemotherapy to the subject.

127.-133. (canceled)

134. A method of treating an autoimmune disease, comprising administering to a subject in need thereof a therapeutically effective amount of the conjugate of claim 26.

135. The method of claim 134, wherein the autoimmune disease is rheumatoid arthritis, multiple sclerosis, or systemic lupus erythematosus.

136.-141. (canceled)
